(19) United States
(12) Patent Application Publication
(10) Pub. No.: US 2014/0213538 A1
(43) Pub. Date: Jul. 31, 2014

(54) LYSOPHOSPHATIDIC ACID RECEPTOR ANTAGONISTS

(71) Applicant: InterMune, Inc., Brisbane, CA (US)

(72) Inventors: Brad Owen Buckman, Oakland, CA (US); John Beamond Nicholas, Redwood City, CA (US); Kumaraswamy Emayan, Albany, CA (US); Scott D. Seiwert, Seattle, WA (US); Shendong Yuan, San Ramon, CA (US)

(21) Appl. No.: 14/156,316

(22) Filed: Jan. 15, 2014

Related U.S. Application Data
(60) Provisional application No. 61/752,884, filed on Jan. 15, 2013.
(60) Provisional application No. 61/764,487, filed on Feb. 13, 2013.
(60) Provisional application No. 61/776,644, filed on Mar. 11, 2013.
(60) Provisional application No. 61/831,097, filed on Jun. 4, 2013.
(60) Provisional application No. 61/847,527, filed on Jul. 17, 2013.
(60) Provisional application No. 61/912,433, filed on Dec. 5, 2013.

(51) Int. Cl. C07D 513/04; C07D 275/03; C07D 413/10; C07H 15/26; C07D 495/04; C07D 491/048; A61K 9/00; C07D 261/14; C07D 487/04

(52) U.S. Cl. CPC C07D 513/04; C07D 261/14; C07D 275/03; C07D 413/10; C07D 487/04; C07D 495/04; C07D 491/048; A61K 9/007; C07H 15/26
USPC 514/27; 548/245; 514/380; 548/214; 514/372; 546/156; 514/312; 544/140; 514/233.8; 544/350; 514/249; 546/114; 514/301; 544/236; 514/248; 548/153; 514/367; 546/115; 514/302; 536/17.4; 435/375

(57) ABSTRACT

Compounds, methods of making such compounds, pharmaceutical compositions and medicaments comprising such compounds, and methods of using such compounds to treat, prevent or diagnose diseases, disorders, or conditions associated with one or more of the lysophosphatidic acid receptors are provided.

### CROSS-REFERENCE TO RELATED APPLICATIONS

The present application claims the benefit of priorities to U.S. Appl. No. 61/752,884, filed Jan. 15, 2013; U.S. Appl. No. 61/764,487, filed Feb. 13, 2013; U.S. Appl. No. 61/776,644, filed Mar. 11, 2013; U.S. Appl. No. 61/831,097, filed Jun. 4, 2013; U.S. Appl. No. 61/847,527, filed Jul. 17, 2013 and U.S. Appl. No. 61/912,433, filed Dec. 5, 2013, all of which are hereby incorporated by reference in their entireties. Any and all applications for which a foreign or domestic priority claim is identified in the Application Data Sheet as filed with the present application, or any correction thereto, are hereby incorporated by reference under 37 CFR 1.57.

### FIELD

Compounds, methods of making such compounds, pharmaceutical compositions and medicaments comprising such compounds, and methods of using such compounds to treat, prevent or diagnose diseases, disorders, or conditions associated with one or more of the lysophosphatidic acid receptors are provided.

### BACKGROUND

Lysophospholipids are membrane-derived bioactive lipid mediators that affect fundamental cellular functions. These cellular functions include, but are not limited to, proliferation, differentiation, survival, migration, adhesion, invasion, and morphogenesis. These cellular functions influence biological processes that include, but are not limited to, neurogenesis, angiogenesis, wound healing, fibrosis, immunity, and carcinogenesis.

Lysophosphatidic acid (LPA) is a lysophospholipid that has been demonstrated to act through sets of specific G protein-coupled receptors (GPCRs) in an autocrine and paracrine fashion. LPA binding to its cognate GPCRs (LPA₁, LPA₂, LPA₃, LPA₄, LPA₅, and LPA₆) activates intracellular signaling pathways to produce a variety of biological responses. Antagonists of the LPA receptors can be employed in the treatment of diseases, disorders, or conditions in which LPA plays a role.

### SUMMARY

Some embodiments disclosed herein include a compound having the structure of Formula (I):

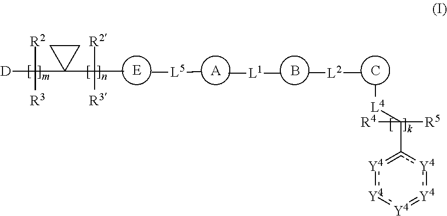

or a pharmaceutically acceptable salt thereof, wherein:

A is an acetylene and B is a ring system selected from the group consisting of 6-11 membered aryl, 5-11 membered heteroaryl, 4-11 membered heterocyclyl, and 4-11 membered carbocyclyl, wherein B is optionally substituted; or alternatively,

B is an acetylene and A is a ring system selected from the group consisting of 6-11 membered aryl, 5-11 membered heteroaryl, 4-11 membered heterocyclyl, and 4-11 membered carbocyclyl, wherein A is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

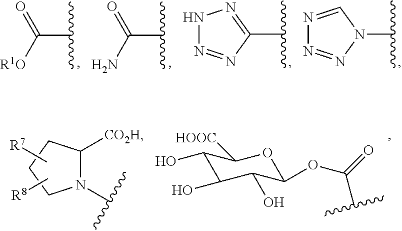

### —NR¹³SOₚR¹⁴, —C(O)—NR¹³SOₚR¹⁴,

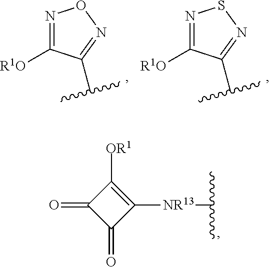

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

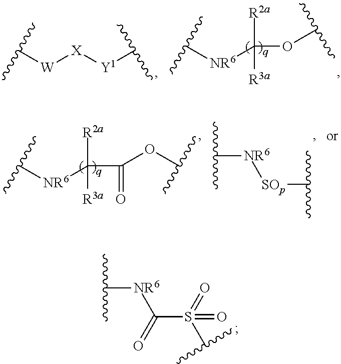

or alternatively,

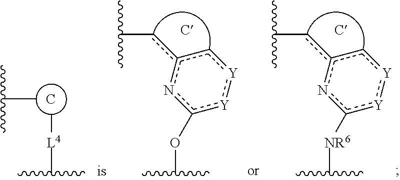

wherein

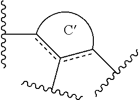

is selected from:

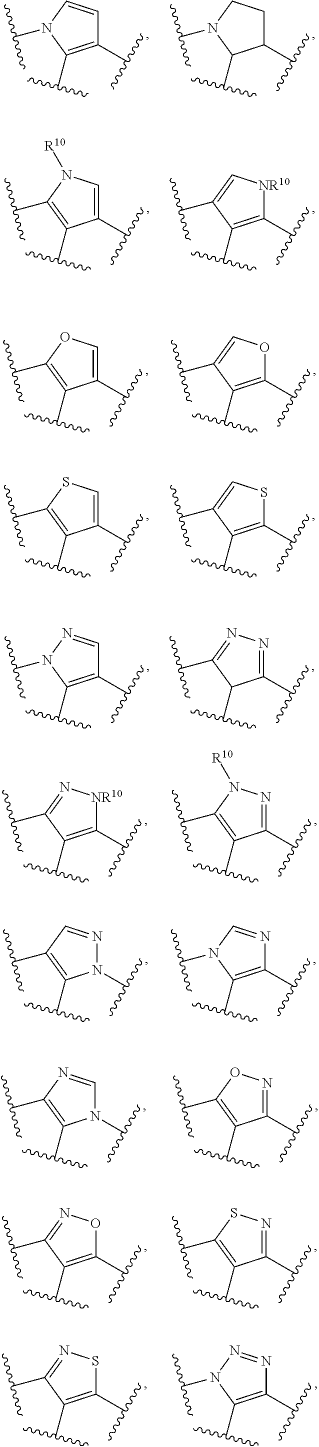

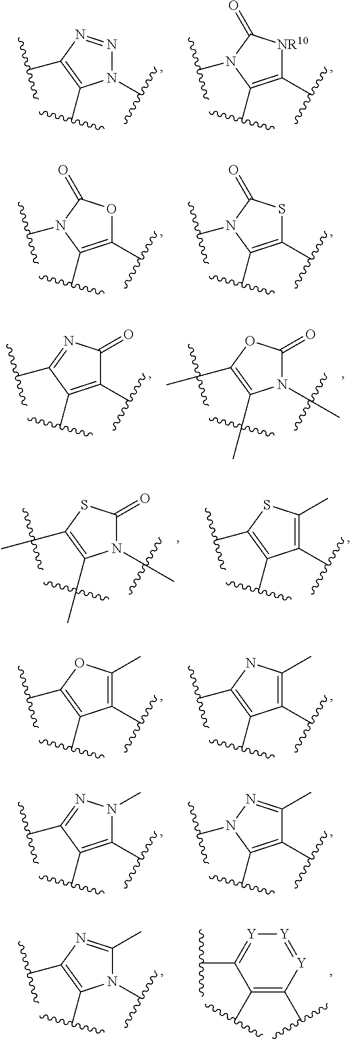

or optionally substituted variants thereof;

L¹ and L² are each independently selected from selected from a single bond, a —CH₂— linker, a —C≡C— linker, a —CH═CH— linker or

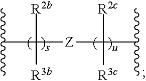

L⁵ is selected from a single bond, a —CH═CH— linker, a —C≡C— linker,

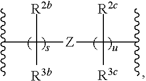

or a 4-7 membered heterocyclyl;

W is C(R⁶)₂, NR⁶, or O;

X is —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R², R³, R^{2′}, and R^{3′} are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R², R³, R^{2′} or R^{3′} is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl; or R^{2′} and R^{3′} are joined together with the atom to which they are attached to form an optionally substituted cycloalkyl or an optionally substituted heterocyclyl;

or R^{2′} is selected from hydrogen, alkyl, halogen, haloalkyl, cycloalkyl, heterocyclyl, aryl, or heteroaryl and R^{3′} is joined to an atom alpha to a point of attachment of L⁵ to A to form an optionally substituted cycloalkyl or an optionally substituted heterocyclyl when E is absent; or R^{3′} is selected from hydrogen, alkyl, halogen, haloalkyl, cycloalkyl, heterocyclyl, aryl or heteroaryl and R^{2′} is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3; provided that the total of m+n is equal to or larger than 1;

k is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (II):

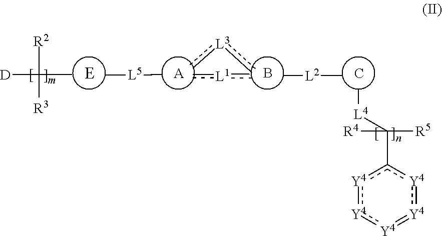

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

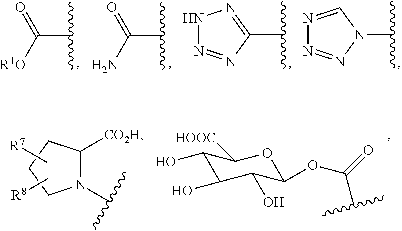

### —NR¹³SOₚR¹⁴, —C(O)—NR¹³SOₚR¹⁴,

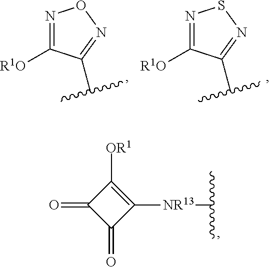

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

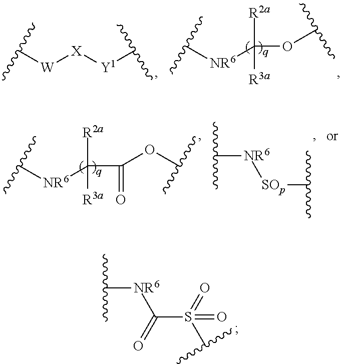

or alternatively,

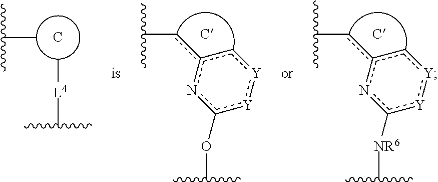

wherein

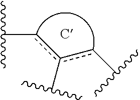

is selected from:

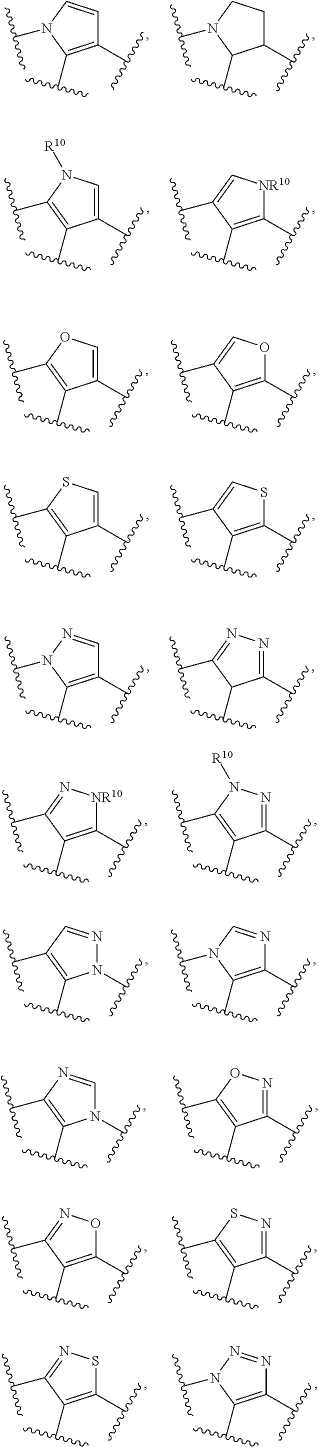

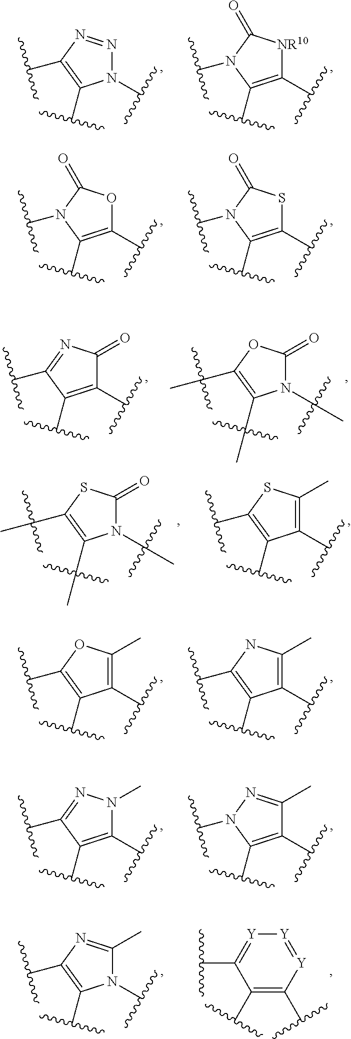

or optionally substituted variants thereof;

L¹ is selected from a single bond, a —CH₂— linker,

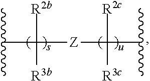

a —C≡C— linker, a —CH═CH— linker, or a ═C(R¹¹)— linker;

L² is selected from a single bond, a —CH₂— linker,

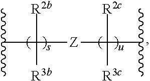

a —C≡C— linker, or a —CH═CH— linker;

L³ is absent or selected from

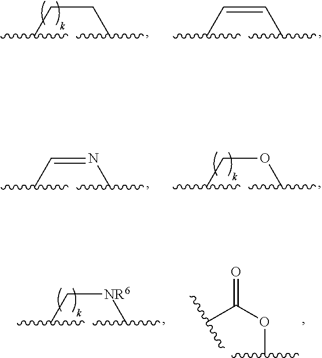

or a ═C(R¹¹)— linker;

L⁵ is selected from

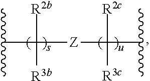

or a —C≡C— linker;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

each s and u is independently an integer from 0 to 6; and

[symbol] represents a single or double bond, provided that

when A is

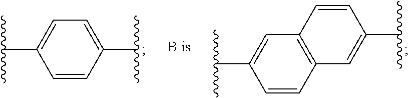 [SMILES: *c1ccc(*)cc1 ; *c1ccc2cc(*)ccc2c1]

D is —C(O)OH; m is 0; E is absent; L⁵ is —CH₂SCH₂CH₂—; L¹ is a single bond; L² is a single bond;

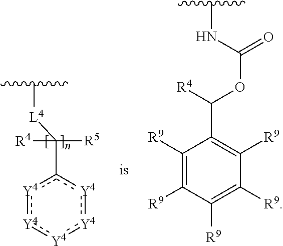

and wherein R⁹ is selected from H or halogen and R⁴ is methyl; then C is not

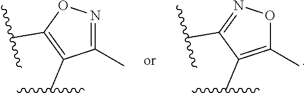

Some embodiments disclosed herein include a compound having the structure of Formula (III):

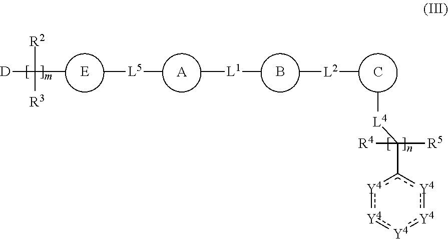

or a pharmaceutically acceptable salt thereof, wherein:

A is selected from

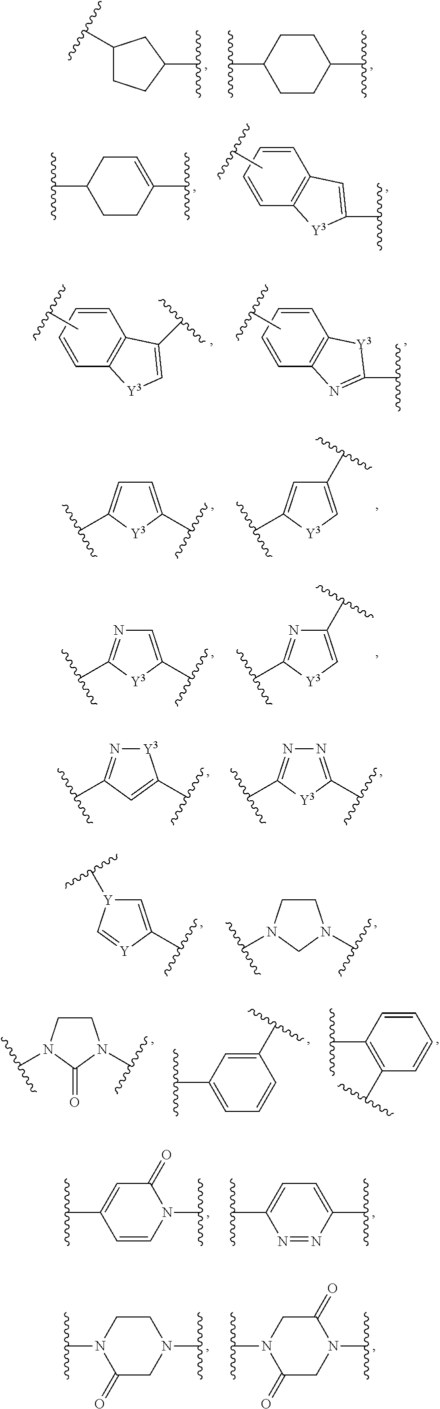

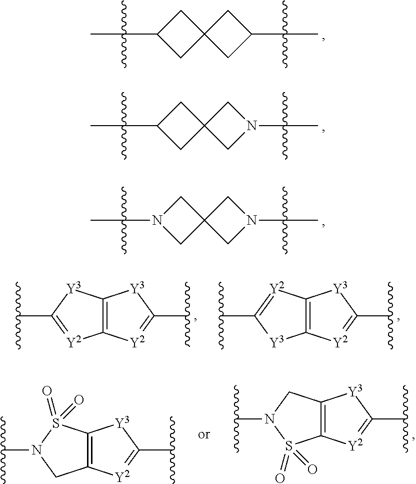

wherein A is optionally substituted; and

B is a ring system selected from the group consisting of 6-11 membered aryl, 5-11 membered heteroaryl, 4-11 membered heterocyclyl, and 4-11 membered carbocyclyl, and wherein B is optionally substituted;

or alternatively,

B is selected from

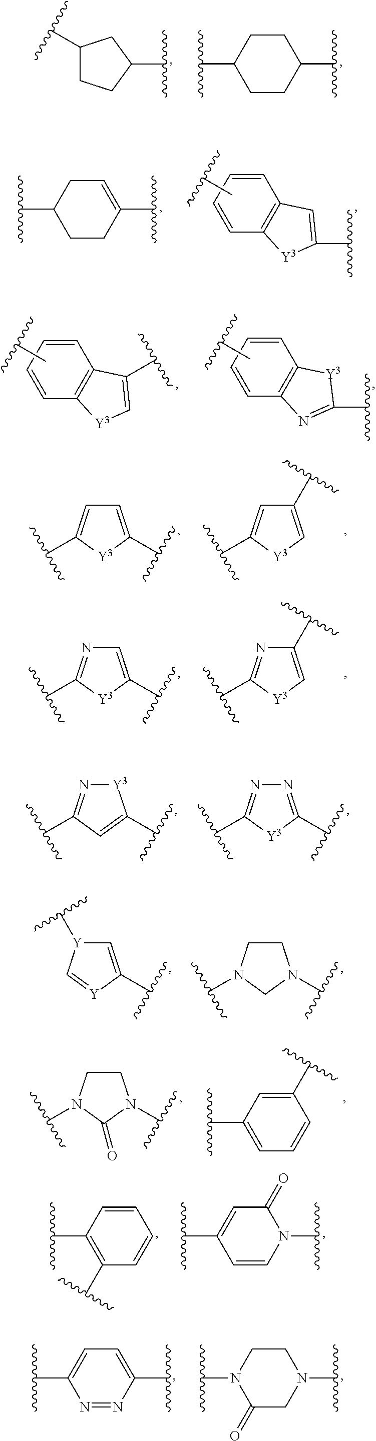

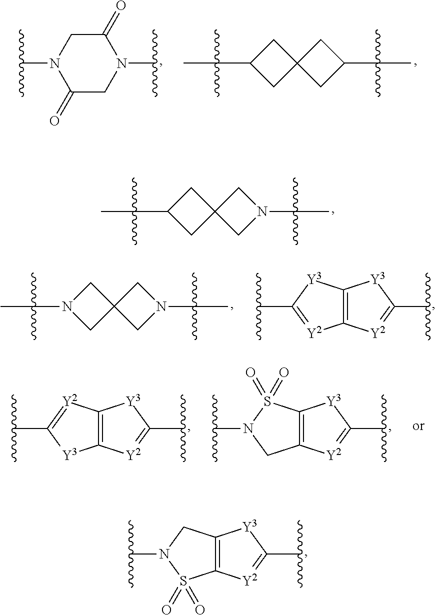

wherein B is optionally substituted; and A is a ring system selected from the group consisting of 6-11 membered aryl, 5-11 membered heteroaryl, 4-11 membered heterocyclyl, and 4-11 membered carbocyclyl, and wherein A is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

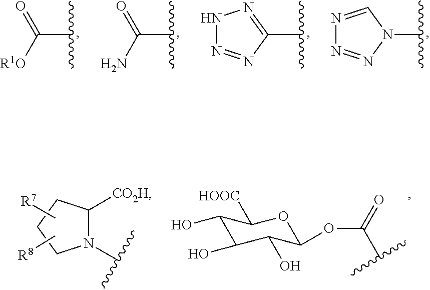

### —NR¹³SOₚR¹⁴, —C(O)—NR¹³SOₚR¹⁴,

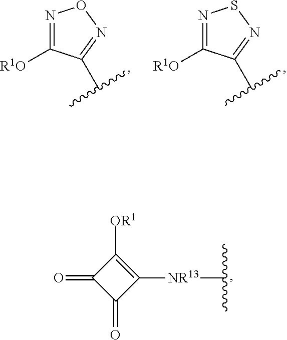

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

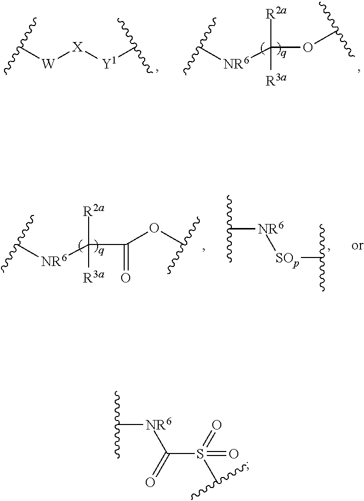

or alternatively,

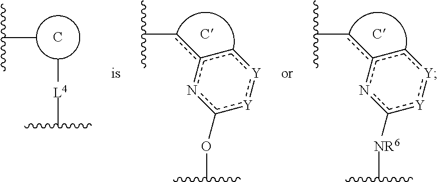

wherein

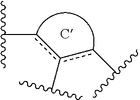

is selected from:

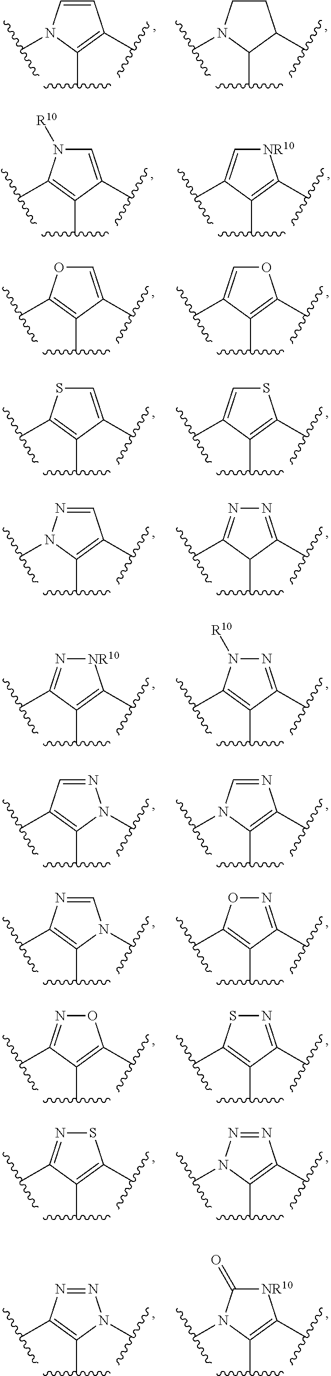

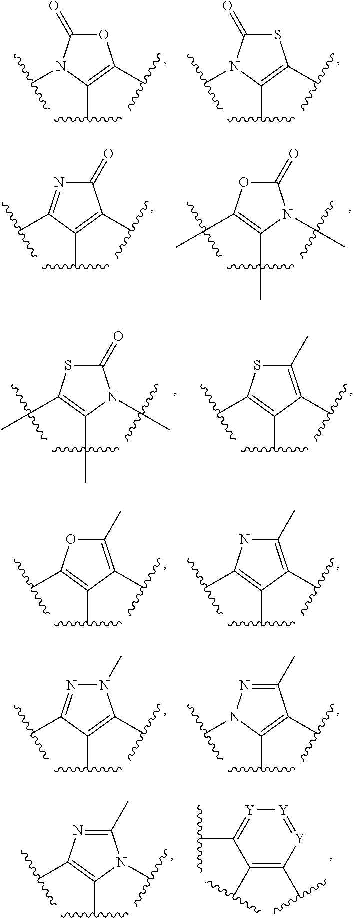

or optionally substituted variants thereof;

L¹ is selected from a single bond,

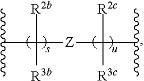

a —C≡C— linker, or a —CH═CH— linker;

L² is selected from a single bond, a —CH₂— linker,

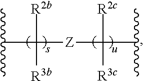

a —C≡C— linker, or a —CH═CH— linker;

L⁵ is selected from a single bond, a —CH═CH— linker, a —C≡C— linker,

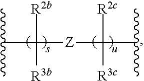

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y² is independently selected from —CH═ or N;

each Y³ is independently selected from C(R⁶)₂, NR⁶, O, or S;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

each s and u is independently an integer from 0 to 6; and

[symbol] represents a single or double bond; provided that

when D is —C(O)OR¹; E is absent; R¹ is hydrogen or alkyl; m is 1; A is

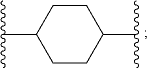 [SMILES: *C1CCC(*)CC1]

B is phenyl; L² is a single bond; L⁵ is a single bond; L¹ is a single bond;

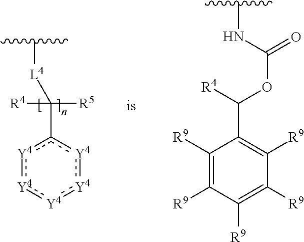

wherein R⁹ is selected from H, alkyl or halogen; then C is not a triazole or pyrazole;

when D is —C(O)OR¹; E is absent; R¹ is hydrogen or alkyl; A is selected from

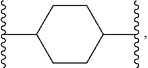 [SMILES: *C1CCC(*)CC1]

B is phenyl; L² is a single bond; L⁵ is a single bond; L¹ is a single bond;

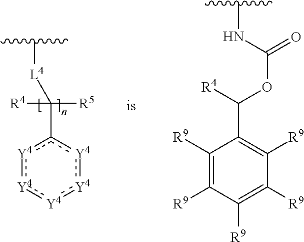

wherein R⁹ is selected from H, alkyl or halogen and R⁴ is methyl; m is 0 or 1; then C is not

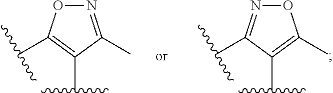

when D is —C(O)OR¹; E is absent; R¹ is hydrogen or alkyl; m is 1 and R² and R³ are both hydrogen; A is phenyl; B is

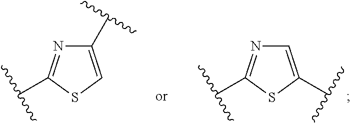 [SMILES: *c1csc(*)n1 ; *c1cnc(*)s1]

L² is a single bond; L⁵ is a single bond; L¹ is a single bond

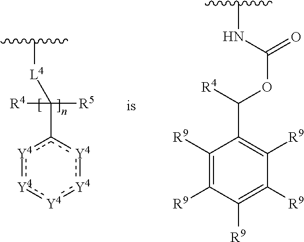

wherein R⁹ is selected from H, alkyl or halogen; then C is not

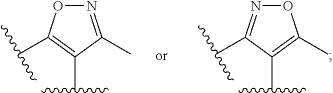

when D is —C(O)OH; E is absent; A is selected from

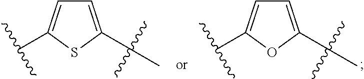 [SMILES: *c1ccc(*)s1 ; *c1ccc(*)o1]

B is phenyl; L² is a single bond; L⁵ is a single bond; L¹ is a single bond;

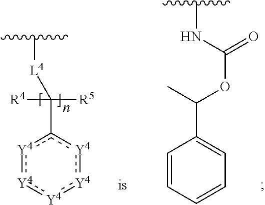

m is 0 or 1; then C is not

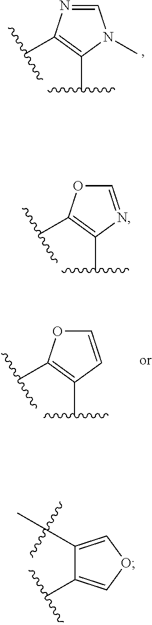

when D is —C(O)OR′; R¹ is hydrogen or alkyl; E is absent; one of A and B is selected from

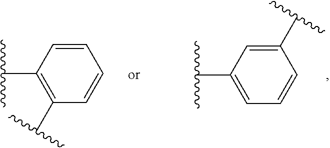 [SMILES: *c1ccccc1* ; *c1cccc(*)c1]

each unsubstituted or substituted with alkyl, halogen or alkoxy; L² is a single bond; L⁵ is a single bond;

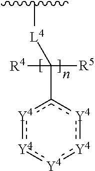

is selected from

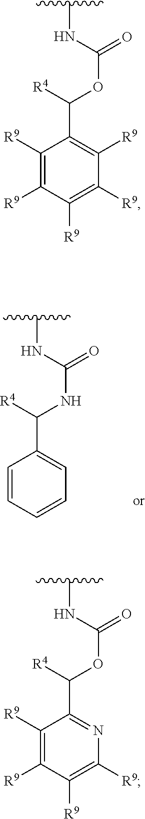

wherein R⁹ is selected from H, alkyl or halogen and R⁴ is hydrogen or methyl; then C is not

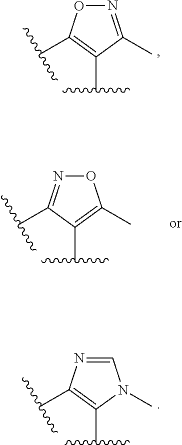

Some embodiments disclosed herein include a compound having the structure of Formula (IV):

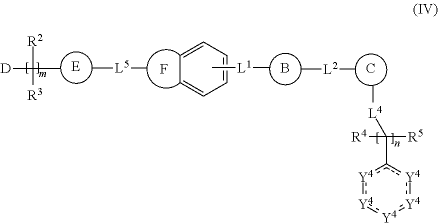

or a pharmaceutically acceptable salt thereof, wherein:

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

E is absent or selected from 6-10 membered arylene, 3-11 membered carbocyclyl, 3-11 membered heterocyclyl or 5 to 10 membered heteroarylene, wherein E is optionally substituted;

F is a 5 or 6 membered heterocyclyl comprising one heteroatom selected from oxygen, nitrogen or sulfur, wherein

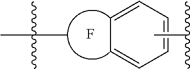

is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

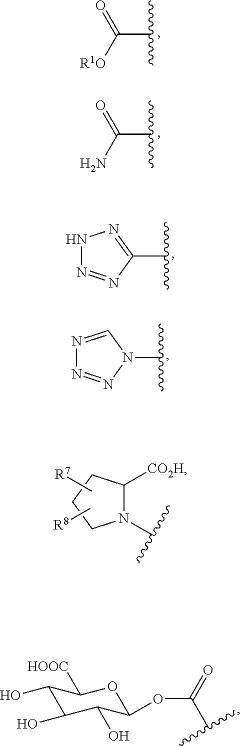

### —NR¹³SOₚR¹⁴, —C(O)—NR¹³SOₚR¹⁴,

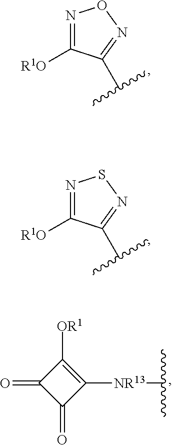

—SOₚR¹⁵, —SOₚNR¹⁶R¹⁷, or carboxylic acid isosteres;

L⁴ is selected from

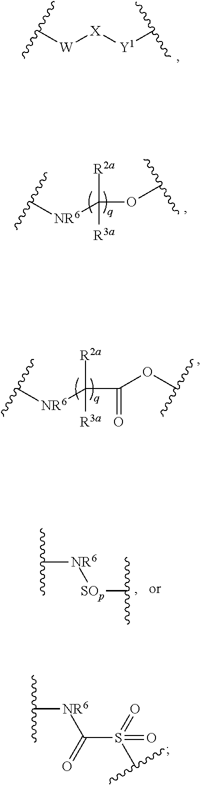

or alternatively,

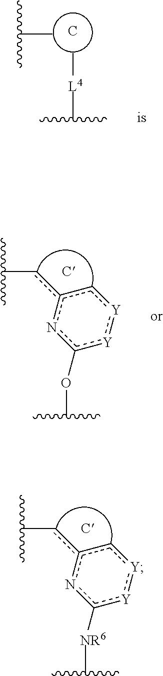

wherein

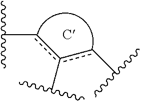

is selected from:

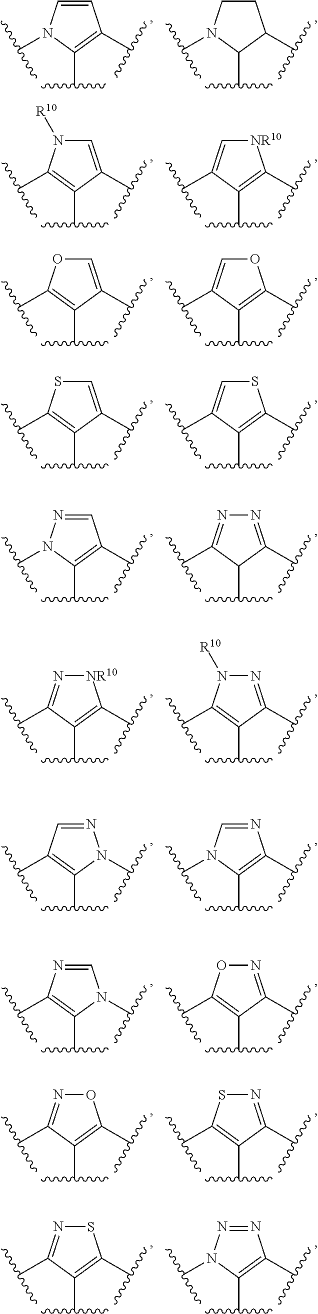

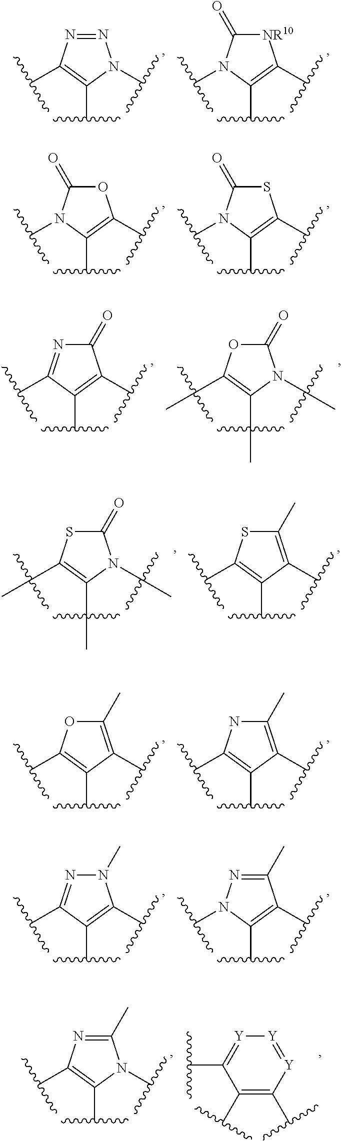

or optionally substituted variants thereof;

L¹ and L² are each independently selected from a single bond, a —CH₂— linker,

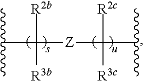

a —C≡C— linker, or a —CH═CH— linker;

L⁵ is selected from a single bond, a —CH═CH— linker, a —C≡C— linker,

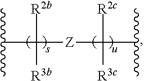

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl; or R² is selected from hydrogen, alkyl, halogen, haloalkyl, cycloalkyl, heterocyclyl, aryl, or heteroaryl and R³ is joined to an atom alpha to a point of attachment of L⁵ to F to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to F to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen, alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (V):

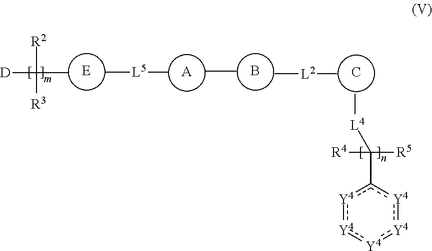

or a pharmaceutically acceptable salt thereof, wherein:

one of A or B is an acetylene and the other A or B is a ring system selected from

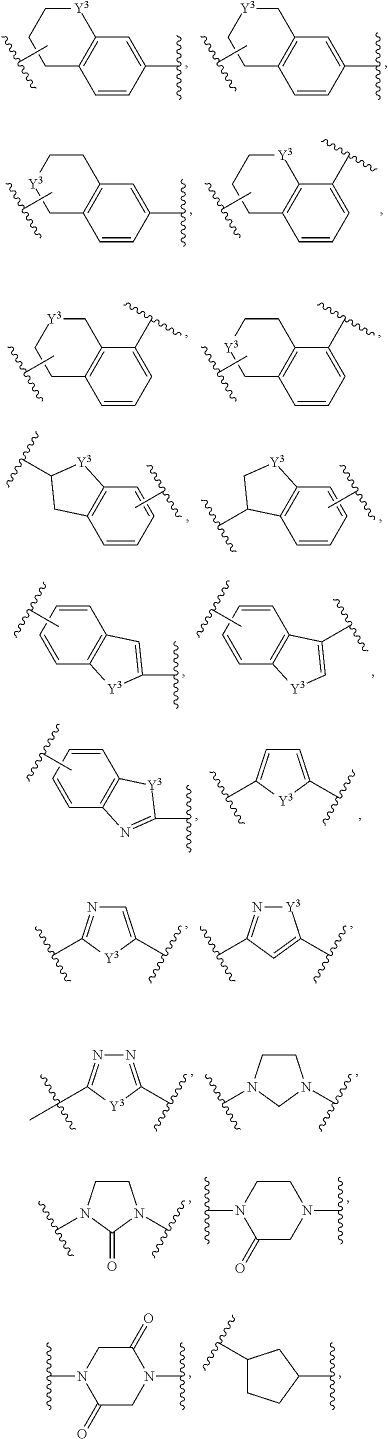

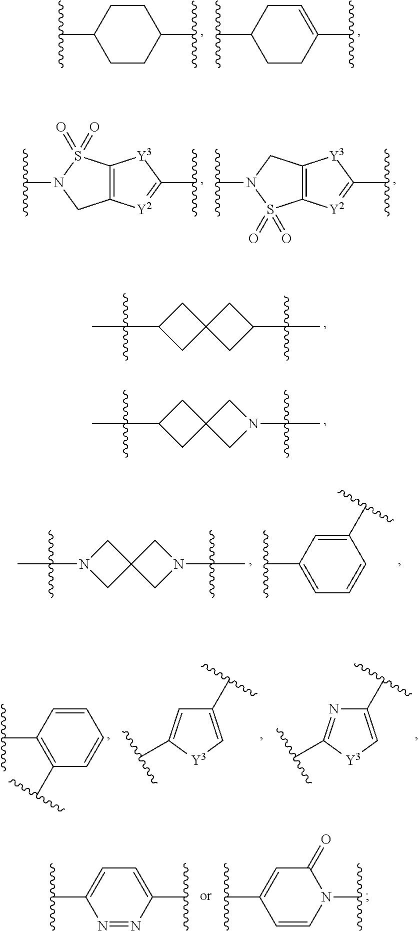

wherein the ring system of A or B is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

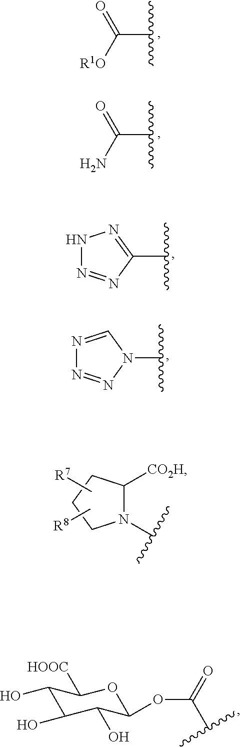

### —NR¹³SOₚR¹⁴, —C(O)—NR¹³SOₚR¹⁴,

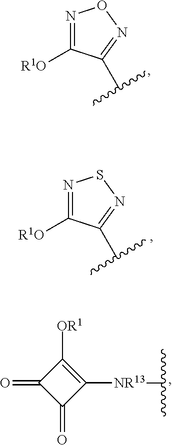

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

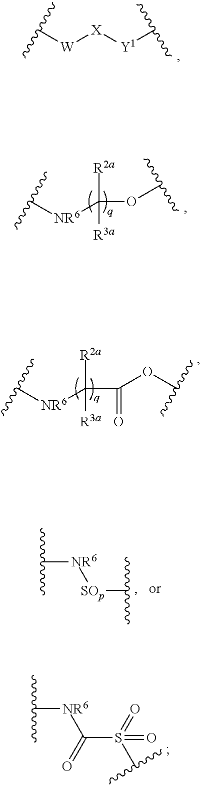

or alternatively,

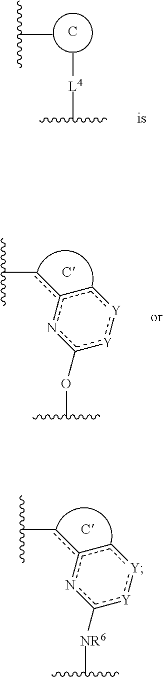

wherein

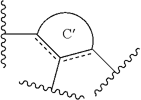

is selected from:

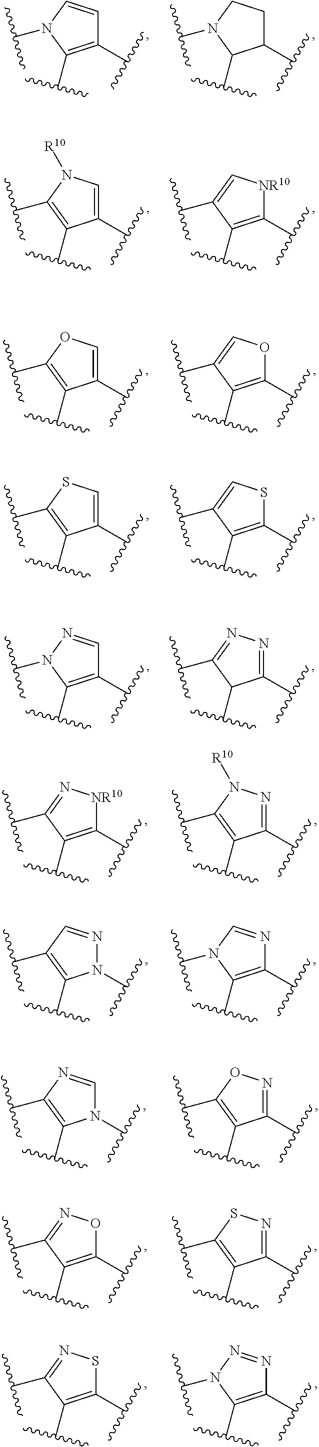

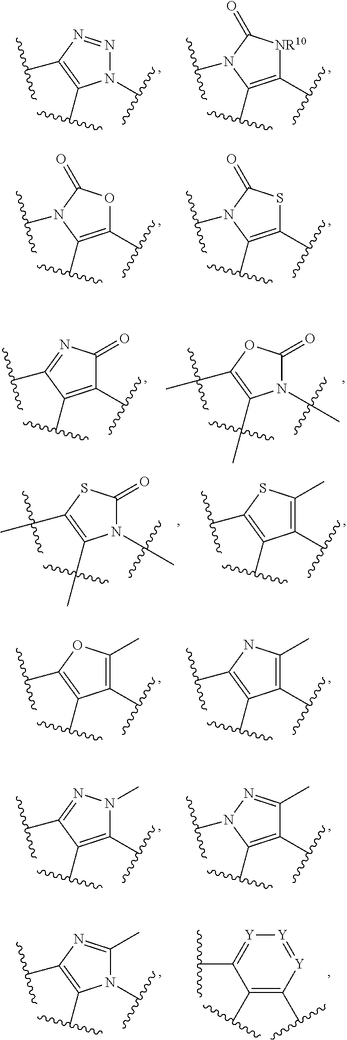

or
optionally substituted variants thereof;

L² is selected from a single bond, a —CH₂— linker,

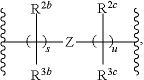

or a —CH═CH— linker;

L⁵ is selected from a single bond, a —CH═CH— linker,

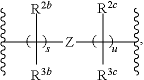

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y³ is independently selected from C(R⁶)₂, NR⁶, O, or S;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent, provided that A is not acetylene;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (VI):

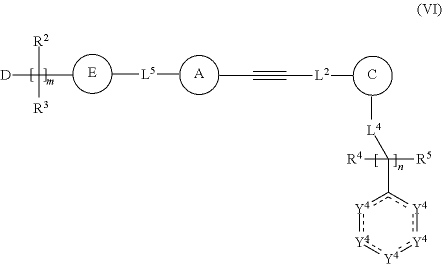

or a pharmaceutically acceptable salt thereof, wherein

A is a ring system selected from the group consisting of 6-11 membered aryl, 5-11 membered heteroaryl, 4-11 membered heterocyclyl, and 4-11 membered carbocyclyl, wherein A is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

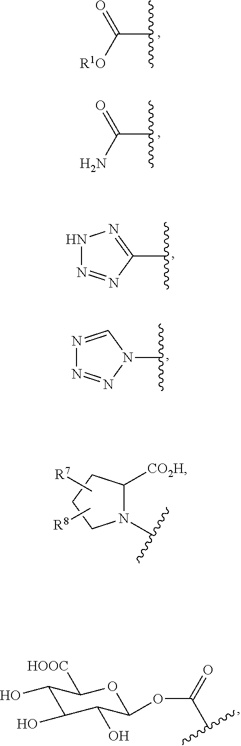

### —NR¹³SOₚR¹⁴, —C(O)—NR¹³SOₚR¹⁴,

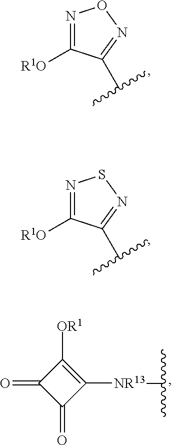

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

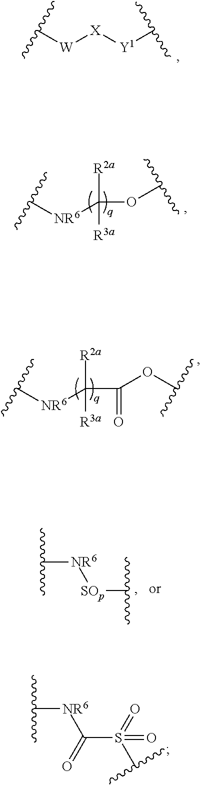

or alternatively,

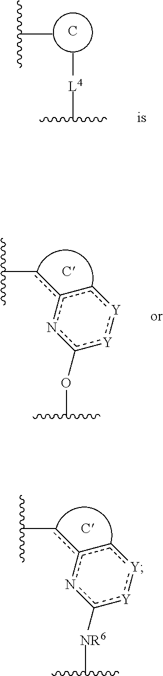

wherein

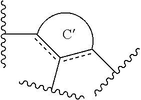

is selected from:

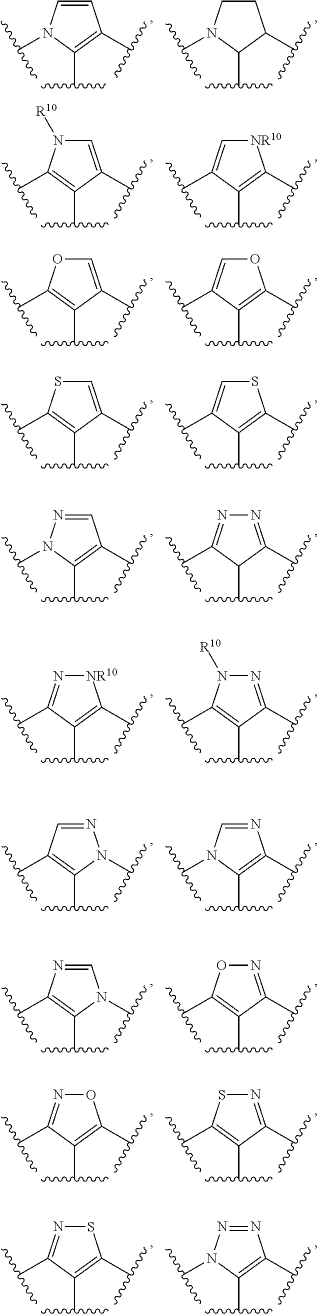

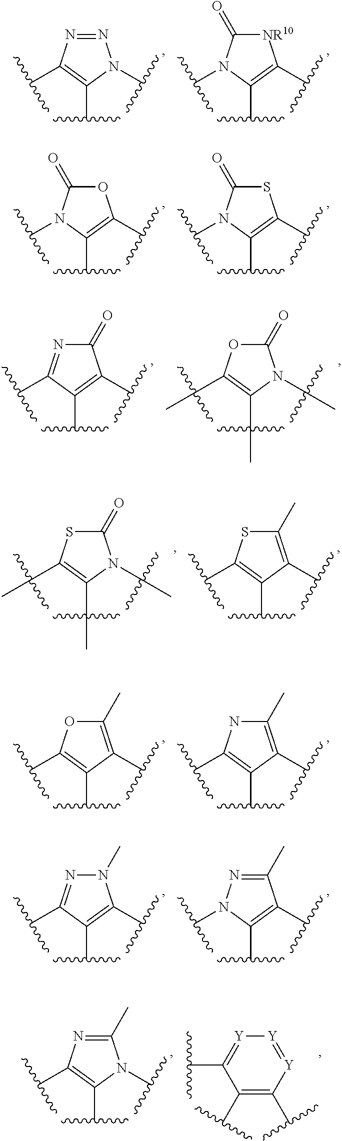

or optionally substituted variants thereof;

L² is selected from a single bond, a —CH₂— linker,

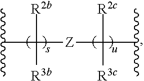

or a —CH═CH— linker;

L⁵ is selected from a —CH═CH— linker or a —C≡C— linker;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

Z is selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

s and u are independently an integer from 0 to 6; and

[symbol] represents a single or double bond.

Some embodiments disclosed herein include a compound having the structure of Formula (VII):

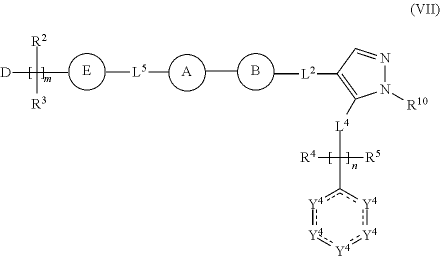

or a pharmaceutically acceptable salt thereof, wherein:

A is an acetylene and B is a ring system selected from the group consisting of 6-11 membered aryl, 5-11 membered heteroaryl, 4-11 membered heterocyclyl, and 4-11 membered carbocyclyl, wherein B is optionally substituted; or alternatively,

B is an acetylene, or is absent when L² is —(CH₂)ₖ— linker, and A is a ring system selected from the group consisting of 6-11 membered aryl, 5-11 membered heteroaryl, 4-11 membered heterocyclyl, and 4-11 membered carbocyclyl, wherein A is optionally substituted; or B is optionally absent when L² is —(CH₂)ₖ— linker;

D is selected from —OH,

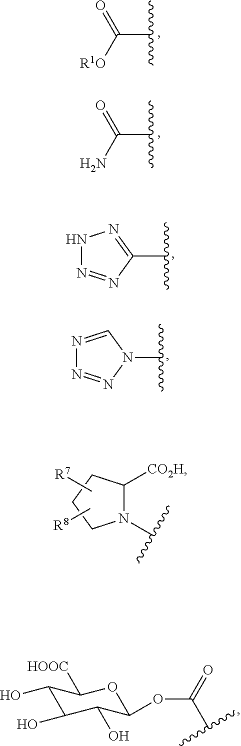

### —NR¹³SOₚR¹⁴, —C(O)—NR¹³SOₚR¹⁴,

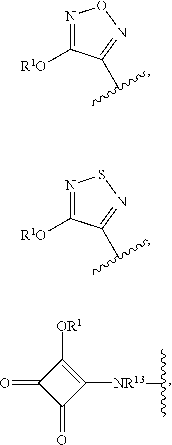

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

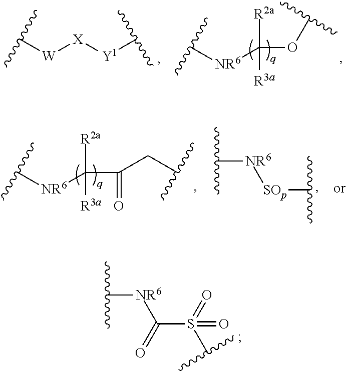

L² is selected from a single bond, a —CH₂— linker, a —(CH₂)ₖ— linker,

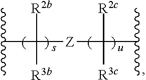

a —C≡C— linker, or a —CH═CH— linker;

L⁵ is selected from a single bond, a —CH═CH— linker, a —C≡C— linker,

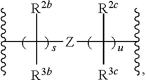

or a 4-7 membered heterocyclyl;

W is selected from C(R⁶)₂, NR⁶, or O;

X is selected from —C(O) or S(O)ₚ;

Y¹ is selected from C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 2-4;

p is an integer from 1-2;

q is an integer from 1-6;

s and u are independently an integer from 0 to 6; and

[symbol] represents a single or double bond.

Some embodiments disclosed herein include a compound having the structure of Formula (VIII):

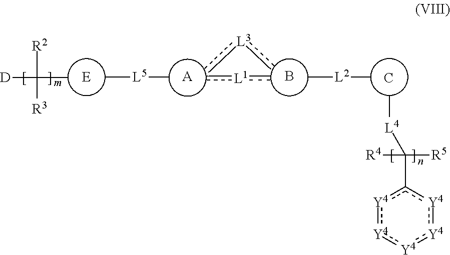

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

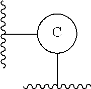

is a ring system selected from the group consisting of

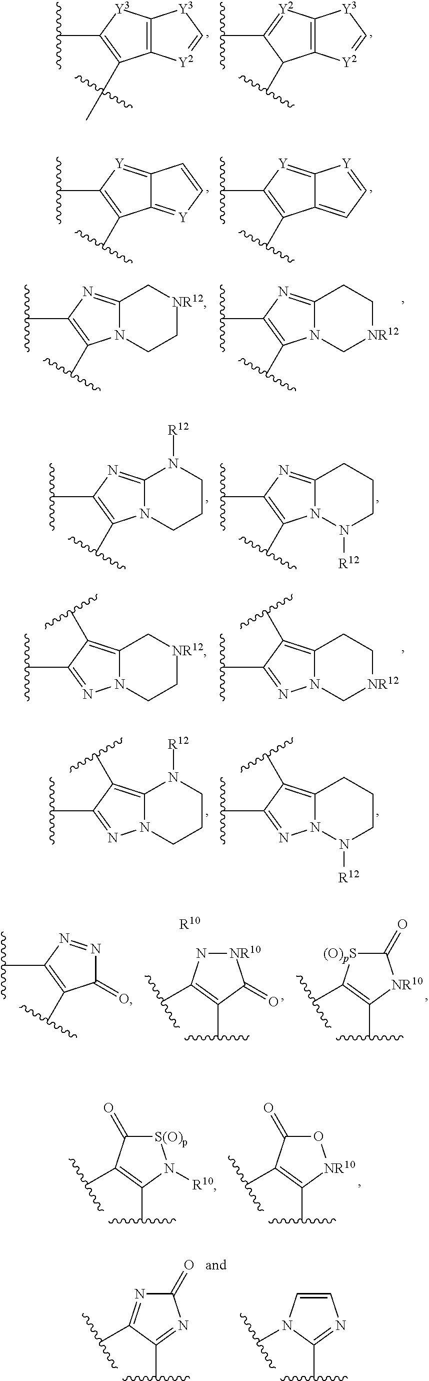

, wherein C is optionally substituted;

D is selected from —OH,

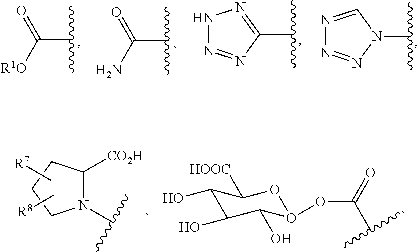

### —NR¹³SOₚR¹⁴, —C(O)—NR¹³SOₚR¹⁴,

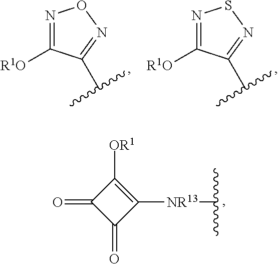

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is

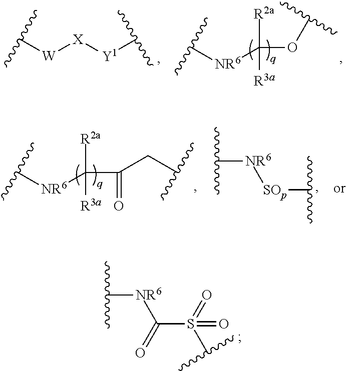

L¹ is selected from a single bond, a —CH₂— linker,

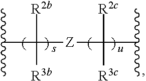

a —C≡C— linker, a —CH═CH— linker, or a ═C(R¹¹)— linker;

L² is selected from a single bond, a —CH₂— linker,

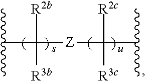

a —C≡C— linker, or a —CH═CH— linker;

L³ is absent,

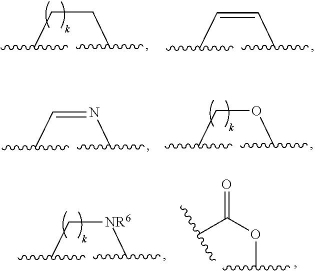

or a ═C(R¹¹)— linker;

L⁵ is selected from a single bond, a —CH═CH— linker, a —C≡C— linker,

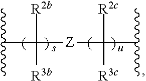

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y² is independently selected from —CH═ or N;

each Y³ is independently selected from C(R⁶)₂, NR⁶, O, or S;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (IX):

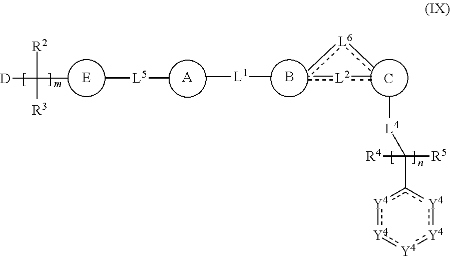

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is a ring system selected from the group consisting of 6-11 membered aryl, 5-11 membered heteroaryl, 4-11 membered heterocyclyl, and 4-11 membered carbocyclyl, wherein B is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

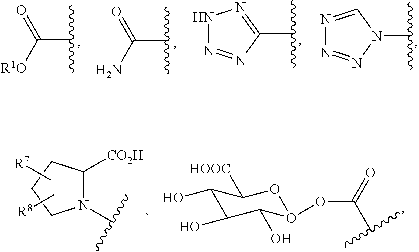

### —NR¹³SOₚR¹⁴, —C(O)—NR¹³SOₚR¹⁴,

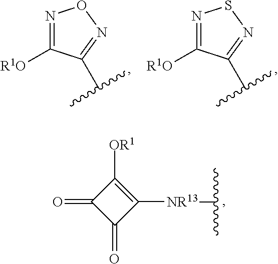

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is

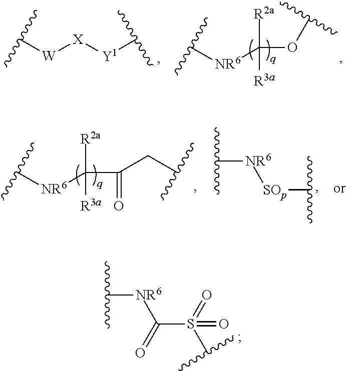

or alternatively,

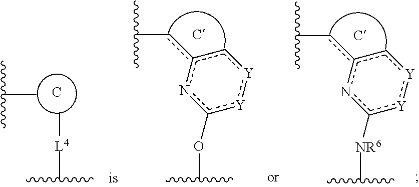

wherein

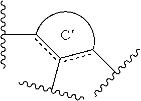

is selected from:

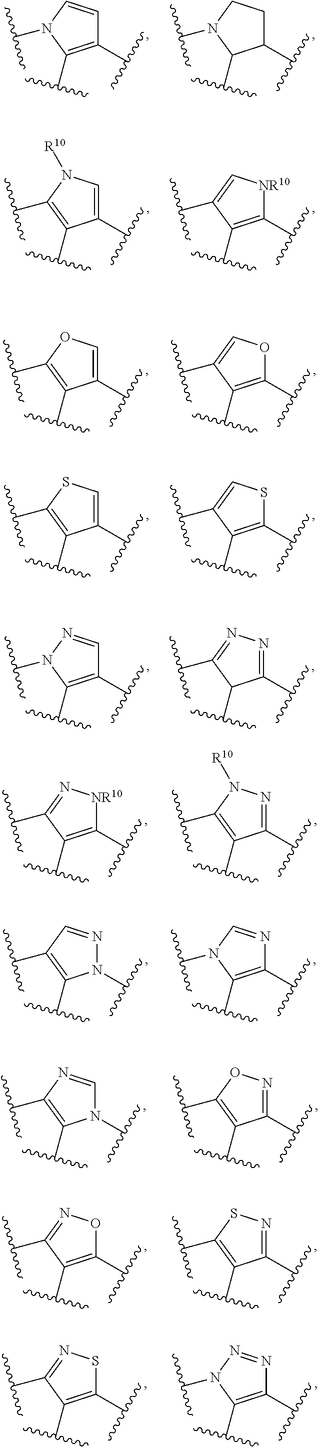

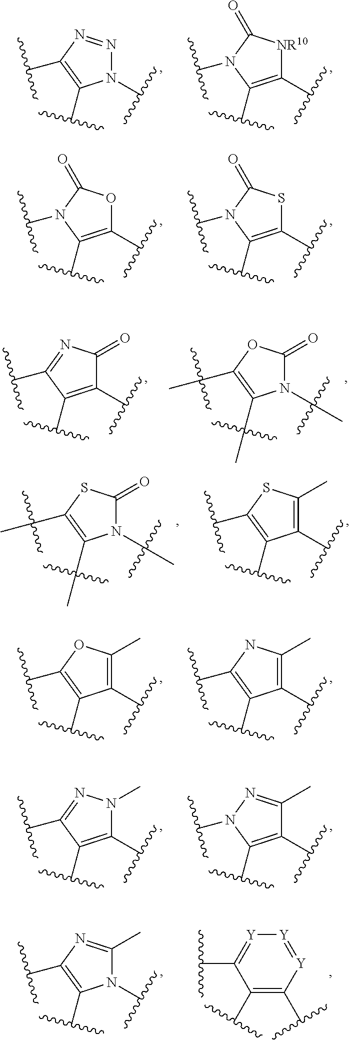

or optionally substituted variants thereof;

L¹ is selected from a single bond, a —CH₂— linker,

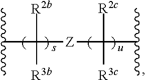

a —C≡C— linker, or a —CH═CH— linker;

L² is selected from a single bond, a —CH₂— linker,

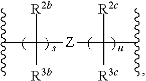

a —C≡C— linker, a —CH═CH— linker, or a ═C(R¹¹)— linker;

L⁵ is selected from a single bond, a —CH═CH— linker, a —C≡C— linker,

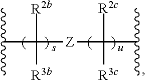

or a 4-7 membered heterocyclyl;

L⁶ is selected from

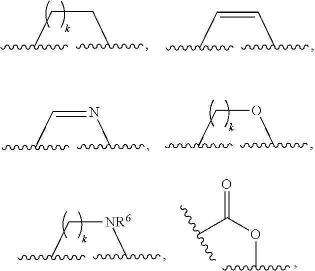

or a ═C(R¹¹)— linker;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R², R³, R^{2′} or R^{3′} is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen; alkyl optionally substituted with one or more substituents selected from the group consisting of halogen, hydroxy and alkoxy; halogen; haloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (X):

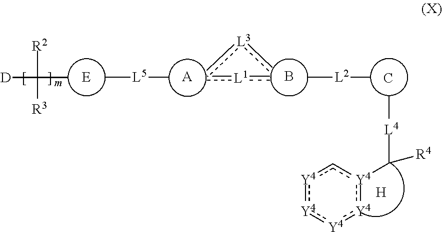

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

H is selected from an optionally substituted 4-11 membered carbocyclyl, an optionally substituted 6-11 membered aryl, an optionally substituted 5-11 membered heteroaryl, or an optionally substituted 4-11 membered heterocyclyl;

D is selected from —OH,

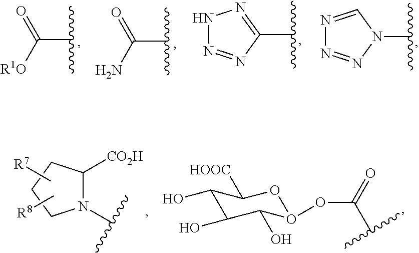

### —NR¹³SOₚR¹⁴, —C(O)—NR¹³SOₚR¹⁴,

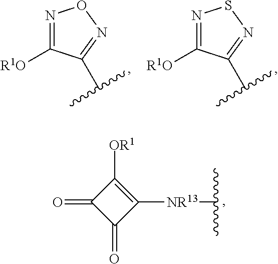

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is

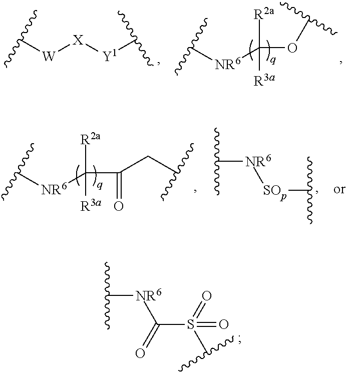

or alternatively,

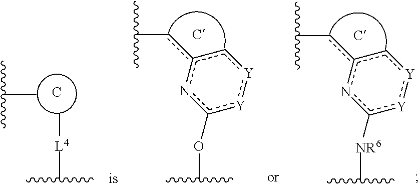

wherein

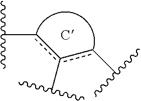

is selected from:

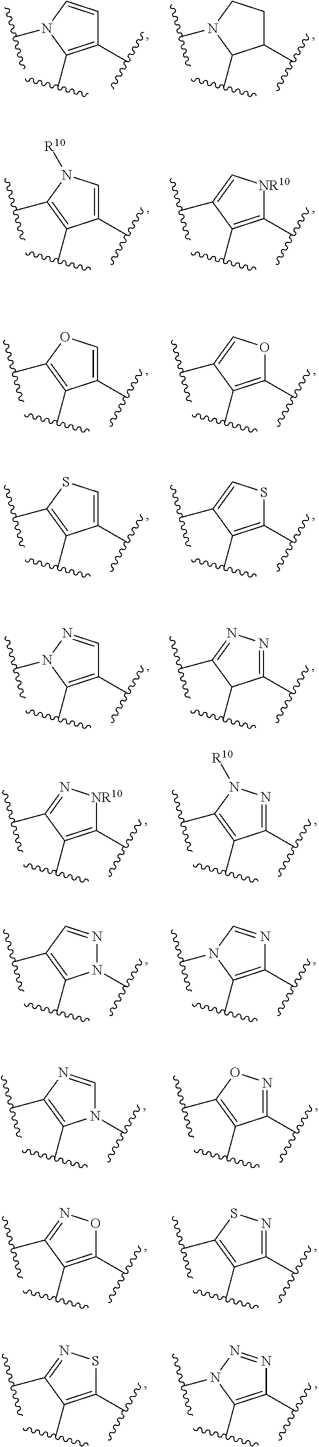

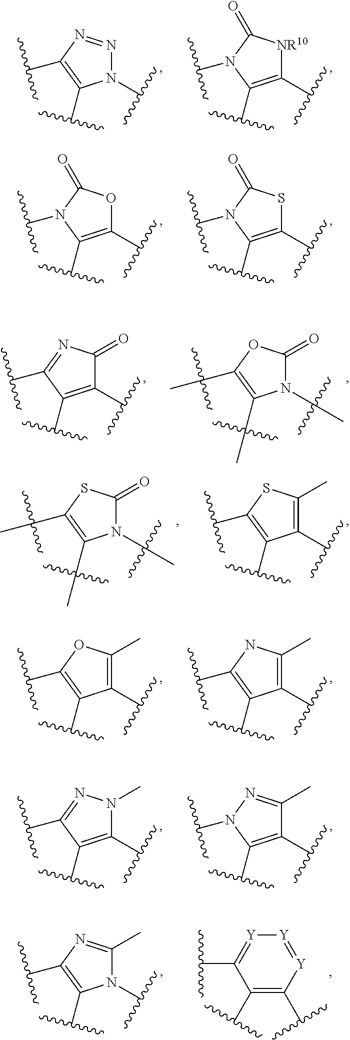

or optionally substituted variants thereof;

L¹ is selected from a single bond, a —CH₂— linker,

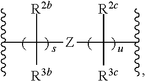

a —C≡C— linker, a ═C(R¹¹)— linker, or a —CH═CH— linker;

L² is selected from a single bond, a —CH₂— linker,

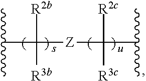

a —C≡C— linker, or a —CH═CH— linker;

L³ is absent or selected from

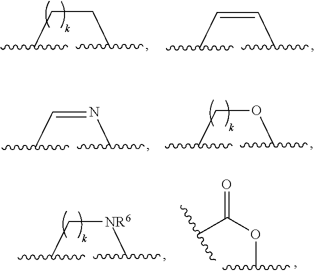

or a ═C(R¹¹)— linker;

L⁵ is selected from a single bond, a —CH═CH— linker, a —C≡C— linker,

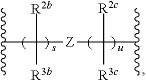

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

R⁴ is selected from hydrogen or alkyl optionally substituted with one or more substituents selected from the group consisting of halogen, hydroxy and alkoxy;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen; alkyl optionally substituted with one or more substituents selected from the group consisting of halogen, hydroxy and alkoxy; halogen; haloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

k is independently an integer from 0-3;

p is an integer from 1-2;

q is an integer from 1-6;

each s and u is independently an integer from 0 to 6; and

[symbol] represents a single or double bond, provided that

when D is —C(O)OH; m is 0; A is

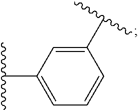 [SMILES: *c1cccc(*)c1]

### B is

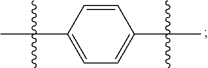 [SMILES: *c1ccc(*)cc1]

each of L¹, L² and L⁵ is a single bond; L³ is absent;

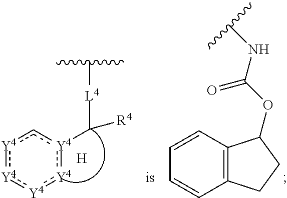

then C is not

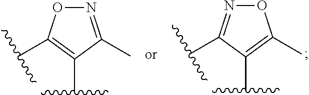

and

when D is —C(O)OH; m is 1 and R² and R³ together with the atom to which they are attached are joined to form a cyclopropyl; both A and B are

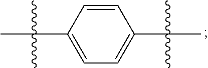 [SMILES: *c1ccc(*)cc1]

each of L¹, L² and L⁵ is a single bond; L³ is absent;

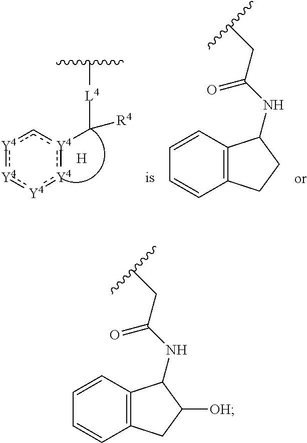

then C is not

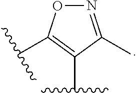 [SMILES: *c1onc(C)c1*]

Some embodiments disclosed herein include a compound having the structure of Formula (XI):

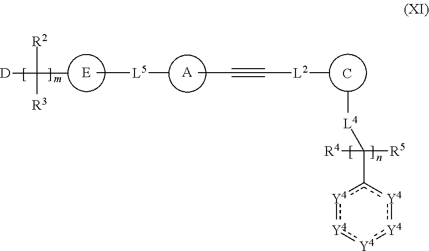

or a pharmaceutically acceptable salt thereof, wherein

A is selected from the group consisting of

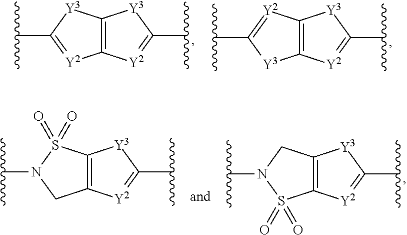

wherein A is optionally substituted;

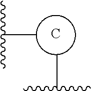

is selected from

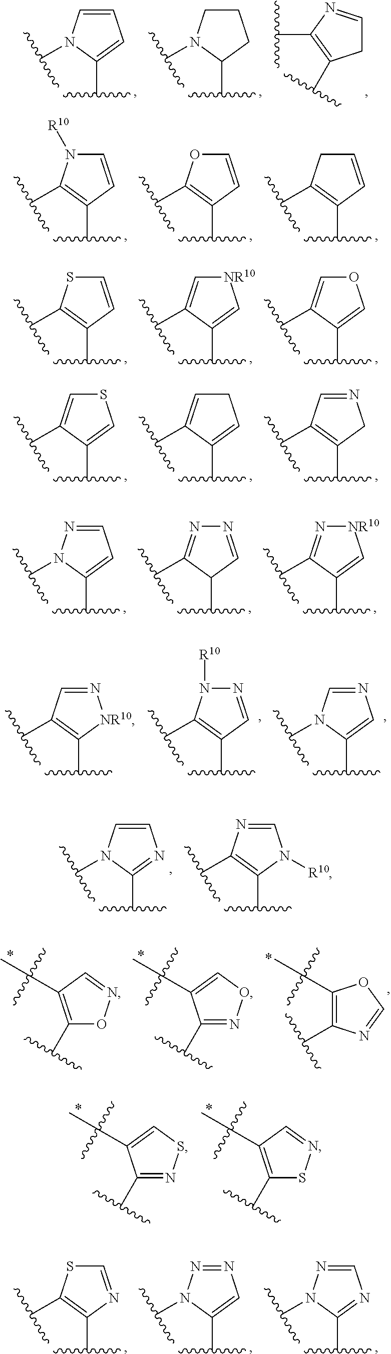

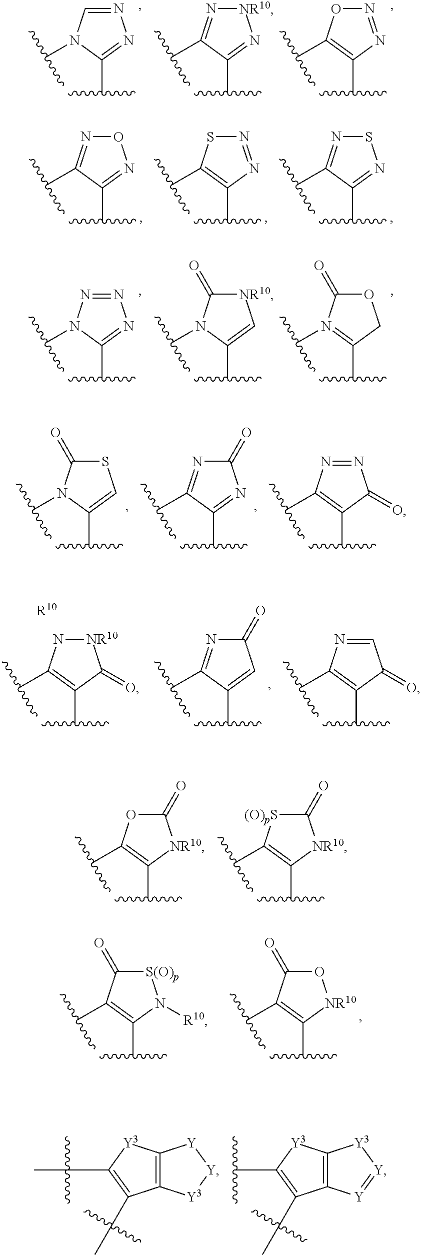

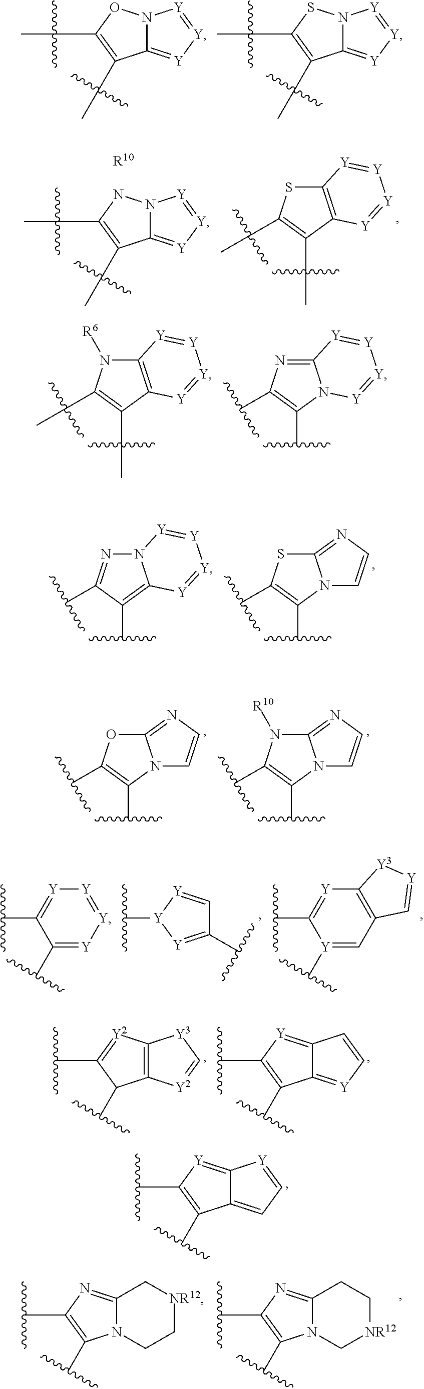

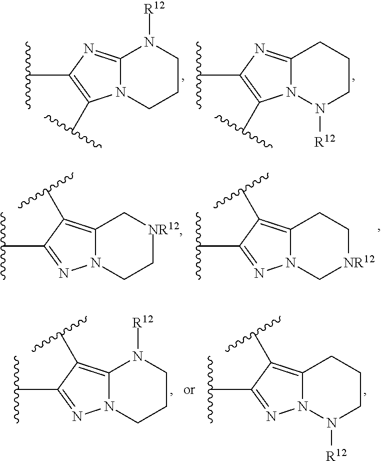

or optionally substituted variants thereof; wherein each * is a point of attachment of C to L²;

D is selected from —OH,

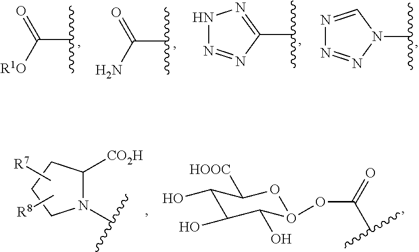

### —NR¹³SOₚR¹⁴, —C(O)—NR¹³SOₚR¹⁴,

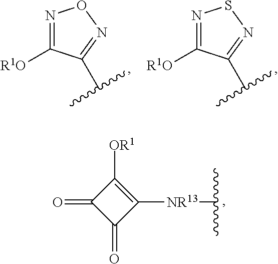

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocvclvl or 5 to 10 membered heteroarylene, wherein E is optionally substituted:

L⁴ is

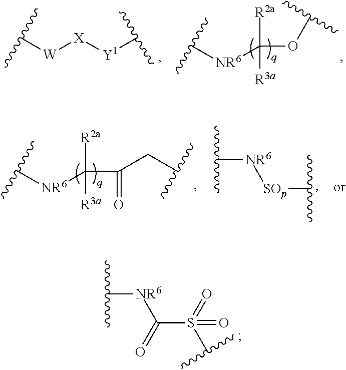

or alternatively,

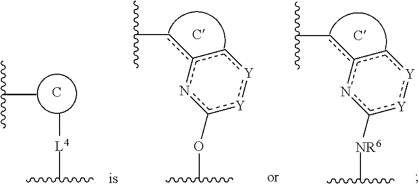

wherein

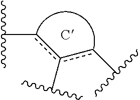

is selected from:

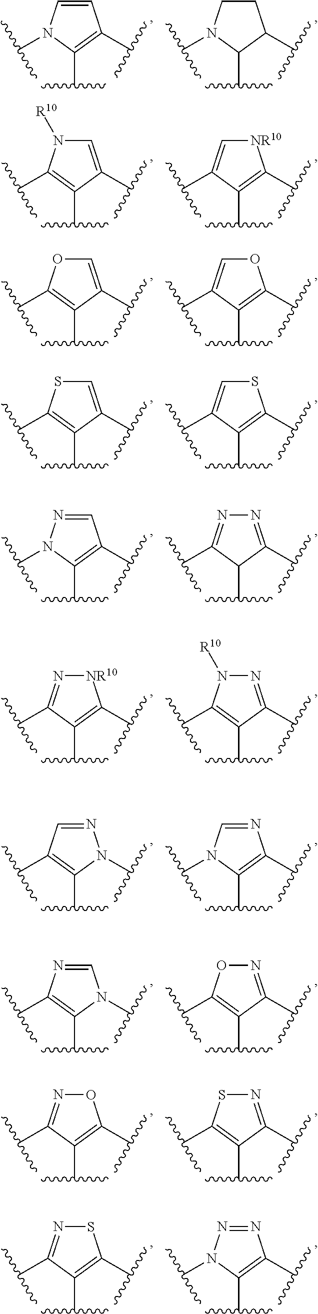

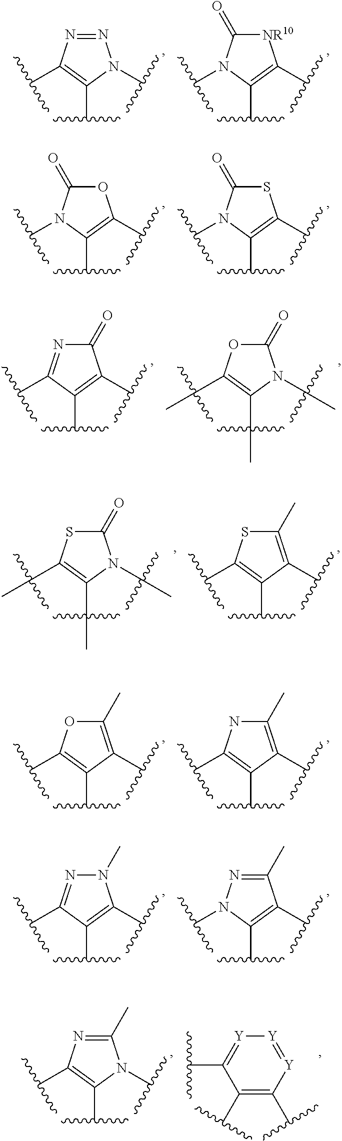

or optionally substituted variants thereof;

L² is selected from a single bond, a —CH₂— linker,

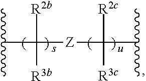

a —C≡C— linker, or a —CH═CH— linker;

L⁵ is selected from a single bond, a —CH═CH— linker, a —C≡C— linker,

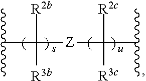

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y² is independently selected from —CH═ or N;

each Y³ is independently selected from C(R⁶)₂, NR⁶, O or S;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen or alkyl optionally substituted with one or more substituents selected from the group consisting of halogen, hydroxy, alkoxy, C-amido, O-carboxy, and 5-7 membered heterocyclyl, or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen, alkyl optionally substituted with one or more substituents selected from the group consisting of halogen, hydroxy and alkoxy, halogen, aryl, or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy, or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen, or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy, halogen, aryl, C₃₋₆ cycloalkyl, or cyano;

each R¹² is independently selected from hydrogen, alkyl optionally substituted with one or more substituents selected from the group consisting of halogen, hydroxy and alkoxy, acyl, C-carboxy, C-amido, sulfinyl, sulfonyl, or S-sulfonamido.

each R¹³ and R¹⁴ is independently selected from hydrogen, alkyl, haloalkyl, halogen, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (XII):

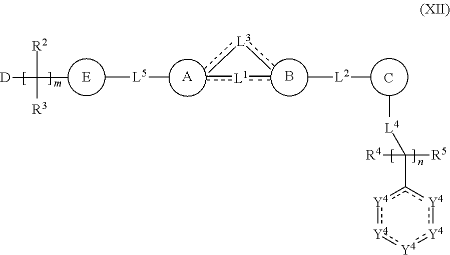

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

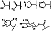

### —NR¹³SOₚR¹⁴, —C(O)—NR¹³SOₚR¹⁴,

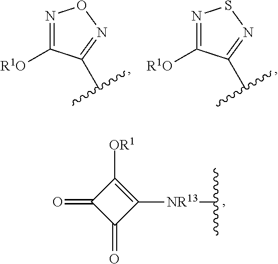

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocvclvl or 5 to 10 membered heteroarylene, wherein E is optionally substituted;

L⁴ is

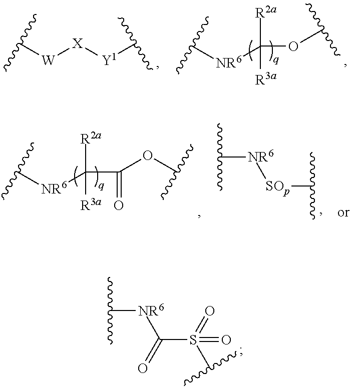

or alternatively,

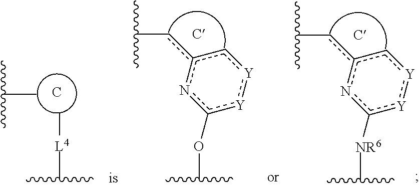

wherein

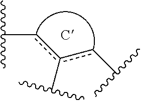

is selected from:

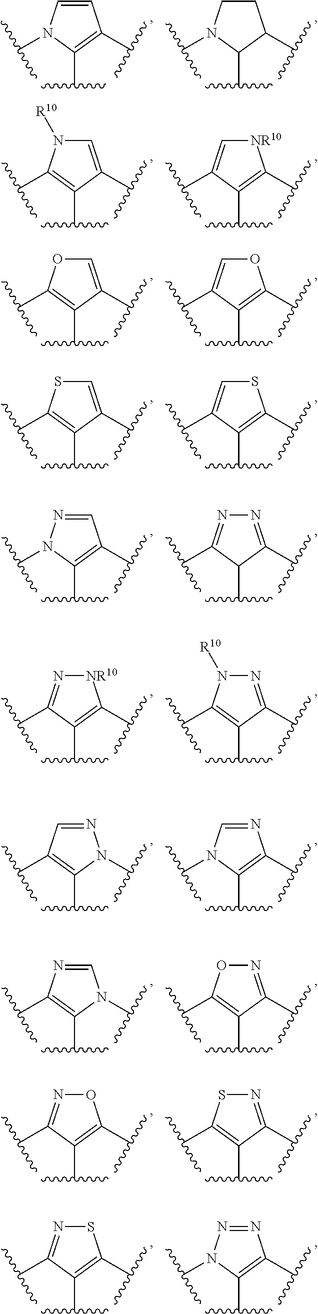

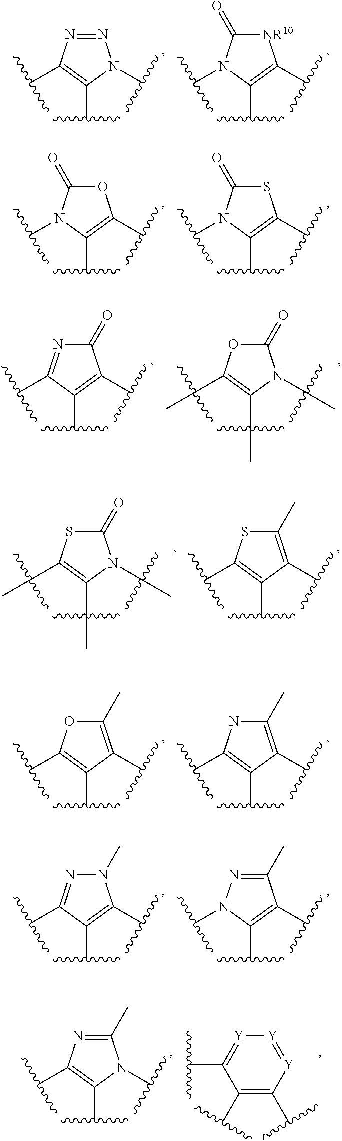

or optionally substituted variants thereof;

L¹ is selected from a single bond, a —CH₂— linker,

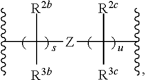

a —C≡C— linker, a —CH═CH— linker, or a ═C(R¹¹)— linker;

L² is selected from a single bond, a —CH₂— linker,

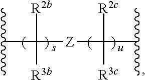

a —C≡C— linker, or a —CH═CH— linker;

L³ is absent or selected from

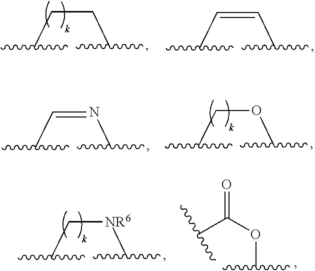

or a ═C(R¹¹)— linker;

L⁵ is selected from a single bond, a —CH═CH— linker, a —C≡C— linker,

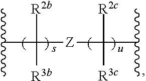

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, halogen, haloalkyl, C₃₋₇ cycloalkyl, 3-7 membered heterocyclyl, or 5-10 membered heteroaryl; wherein each C₃₋₇ cycloalkyl, 3-7 membered heterocyclyl, and 5-10 membered heteroaryl of R² or R³ is optionally substituted; provided that R² and R³ cannot both be hydrogen;

or R² and R³ are joined together with the atom to which they are attached to form a halo-substituted C₃₋₇ cycloalkyl or halo-substituted 3-7 membered heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 1-3;

n is an integer from 0-3;

k is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (XIII):

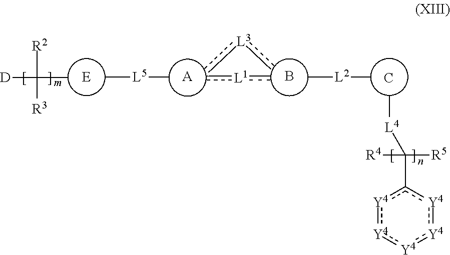

or a pharmaceutically acceptable salt thereof, wherein:

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —SOₚR¹⁵ or —SOₚNR¹⁶R¹⁷;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

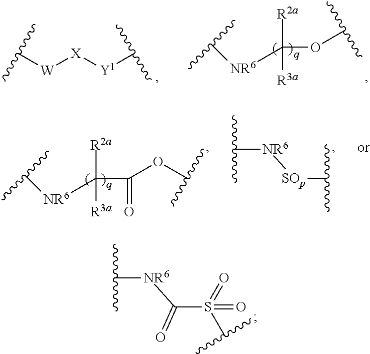

or alternatively,

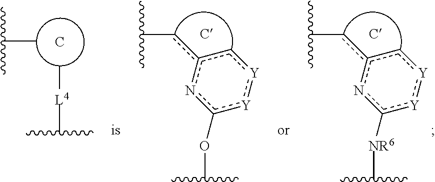

wherein

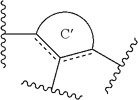

is selected from:

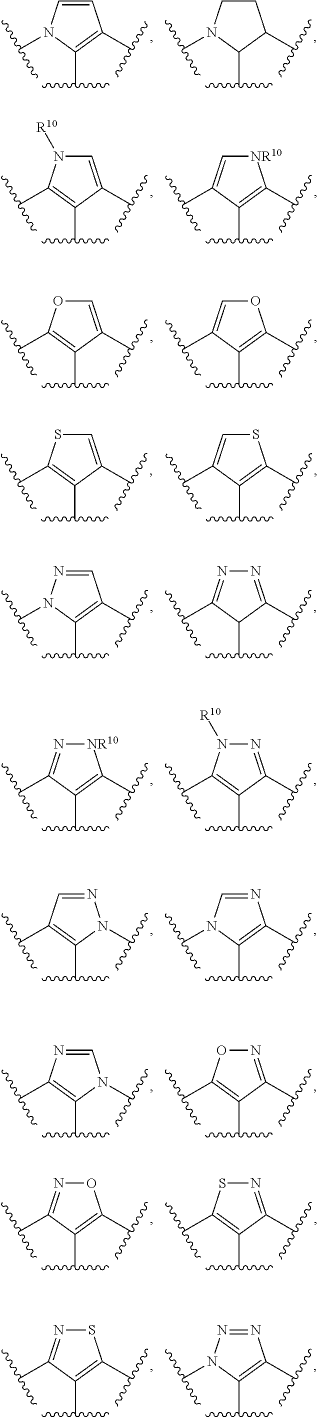

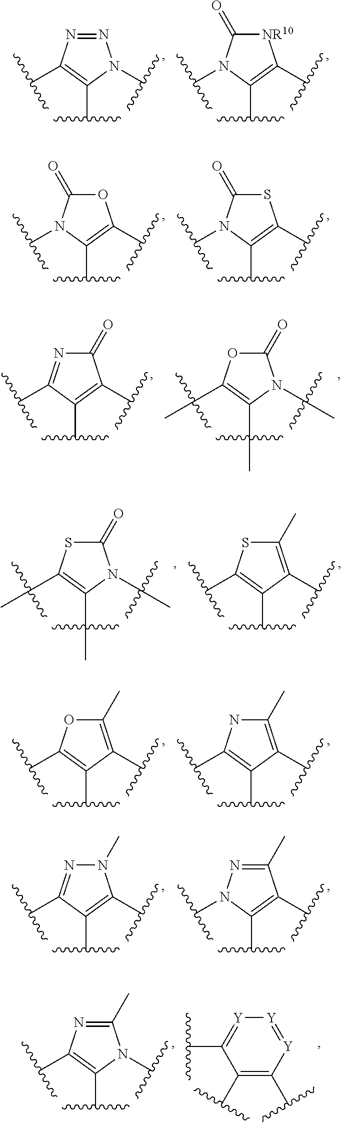

or optionally substituted variants thereof;

L¹ is selected from a single bond, a —CH₂— linker,

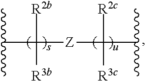

a —C≡C— linker, a —CH═CH— linker, or a ═C(R¹¹)— linker;

L² is selected from a single bond, a —CH₂— linker,

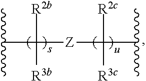

a —C≡C— linker, or a —CH═CH— linker;

L³ is absent or selected from

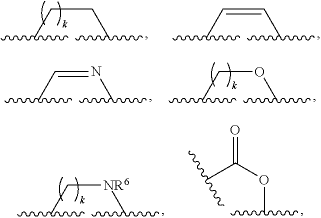

or a ═C(R¹¹)— linker;

L⁵ is selected from a single bond, a —CH═CH— linker, a —C≡C— linker,

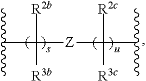

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

each s and u is independently an integer from 0 to 6; and

[symbol] represents a single or double bond.

Some embodiments disclosed herein include a compound having the structure of Formula (XIV):

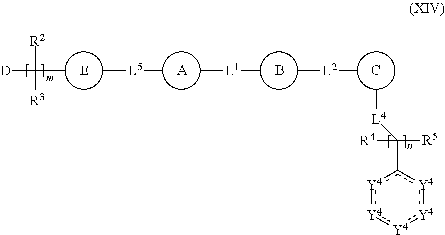

or a pharmaceutically acceptable salt thereof, wherein

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

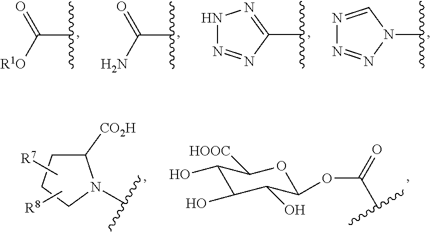

### —NR¹³SOₚR¹⁴, —C(O)—NR¹³SOₚR¹⁴,

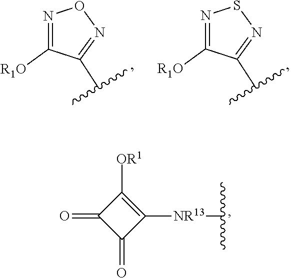

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is

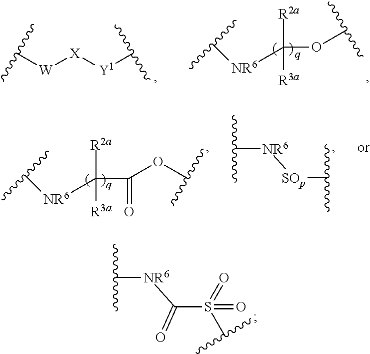

or alternatively,

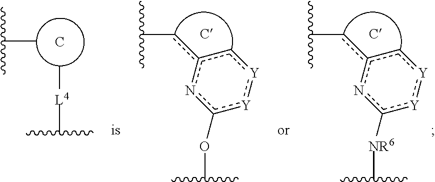

wherein

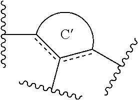

is selected from:

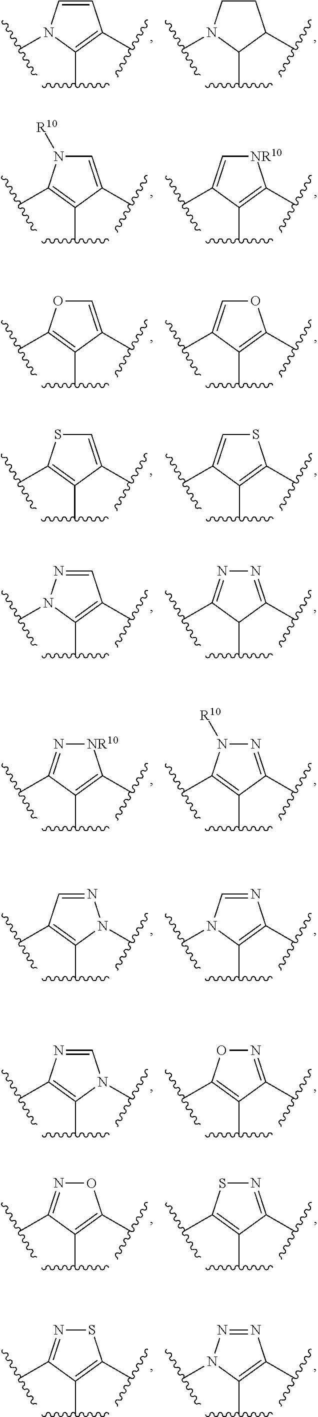

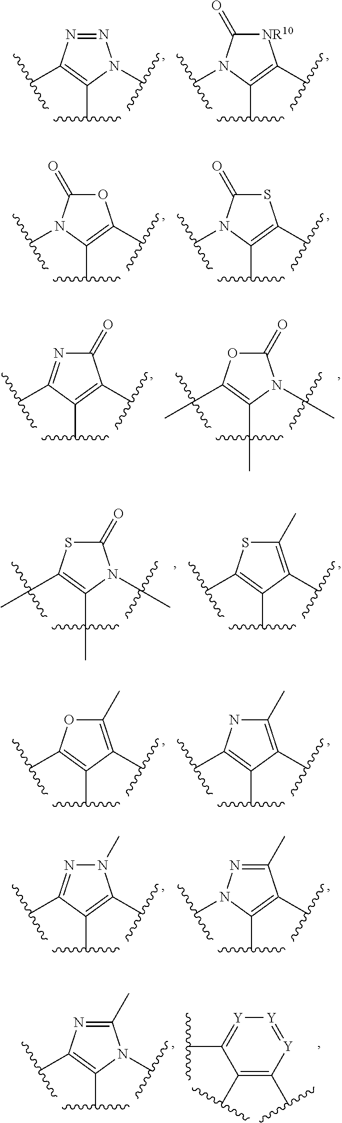

or optionally substituted variants thereof;

L¹ is

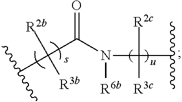

L² is selected from a single bond, a —CH₂— linker,

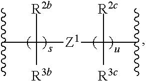

a —C≡C— linker, or a —CH═CH— linker;

L⁵ is selected from a single bond, a —CH═CH— linker, a —C≡C— linker,

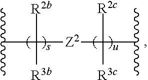

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is C(R⁶)₂, NR⁶, or O;

each Y⁴ is independently absent, CR⁹, C(R⁹)₂, N, or NH, provided that only one Y⁴ can be absent;

Z¹ is independently selected from C(O), NR^{6a}, C(O)NR^{6b}, or S(O)₂NR^{6c};

Z and Z² are each independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl is optionally substituted with one or more substituents selected from alkyl, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

ach s and u is independently an integer from 0 to 6; and

[symbol] represents a single or double bond.

### Formula (XV)

Some embodiments disclosed herein include a compound having the structure of Formula (XV):

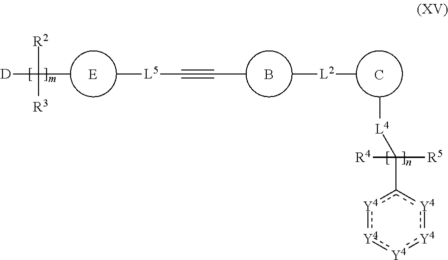

or a pharmaceutically acceptable salt thereof, wherein

B is selected from the group consisting of

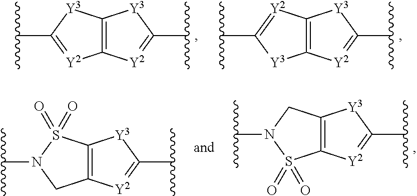

wherein B is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

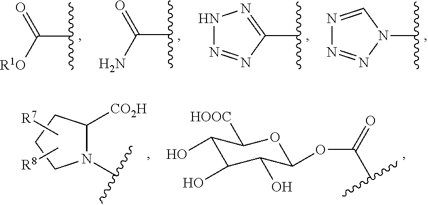

### —NR¹³SOₚR¹⁴, —C(O)—NR¹³SOₚR¹⁴,

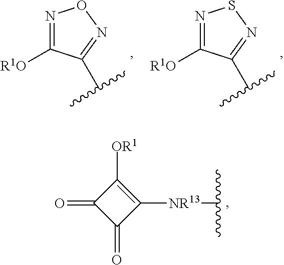

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

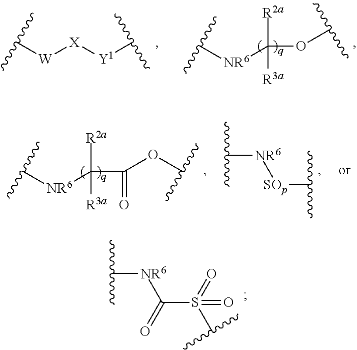

or alternatively,

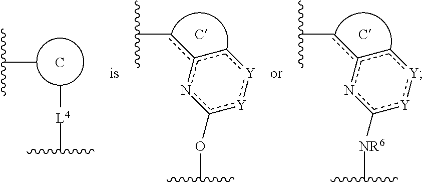

wherein

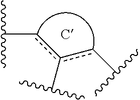

is selected from:

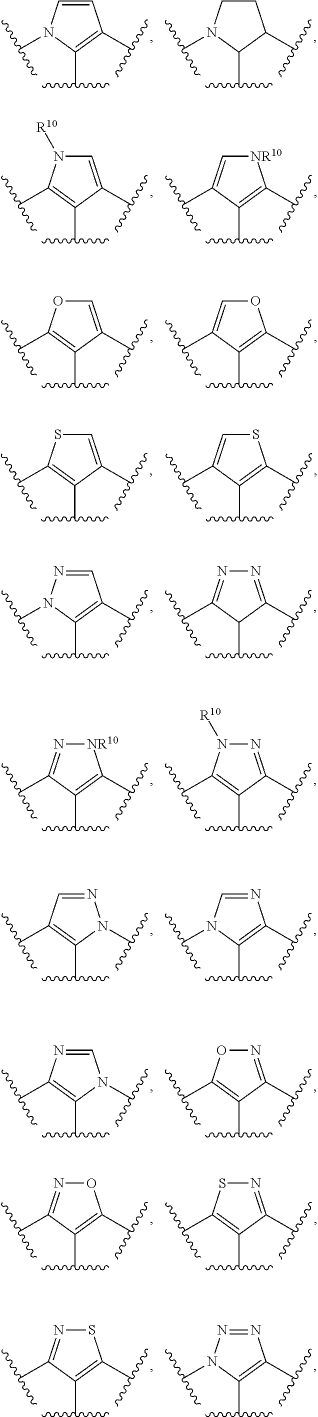

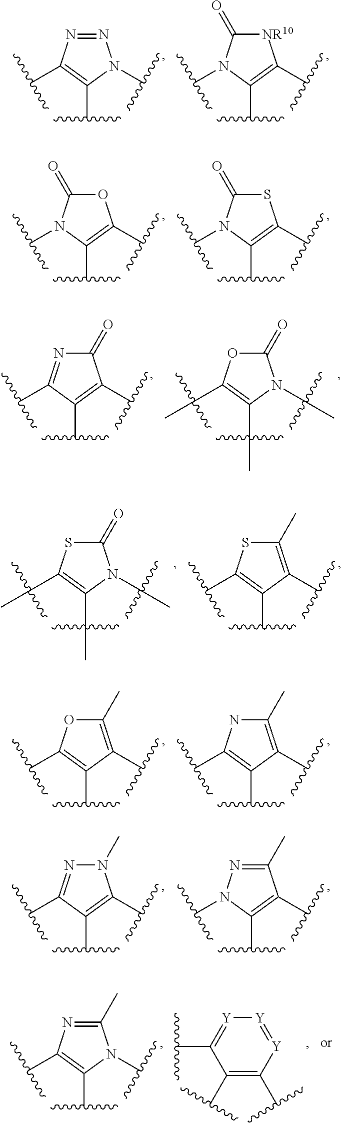

optionally substituted variants thereof;

L² is selected from a single bond, a —CH₂— linker,

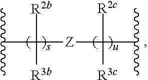

a —C≡C— linker, or a —CH═CH— linker;

L⁵ is selected from a single bond, a —CH═CH— linker, a —C≡C— linker,

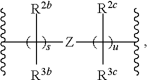

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y² is independently selected from —CH═ or N;

each Y³ is independently selected from C(R⁶)₂, NR⁶, O or S;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen or alkyl optionally substituted with one or more substituents selected from the group consisting of halogen, hydroxy, alkoxy, C-amido, O-carboxy, and 5-7 membered heterocyclyl, or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen, alkyl optionally substituted with one or more substituents selected from the group consisting of halogen, hydroxy and alkoxy, halogen, aryl, or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy, or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen, or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy, halogen, aryl, C₃₋₆ cycloalkyl, or cyano;

each R¹³ and R¹⁴ is independently selected from hydrogen, alkyl, haloalkyl, halogen, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

p is an integer from 1-2;

q is an integer from 1-6;

each s and u is independently an integer from 0 to 6; and

[symbol] represents a single or double bond.

### Formula (XVI)

Some embodiments disclosed herein include a compound having the structure of Formula (XVI):

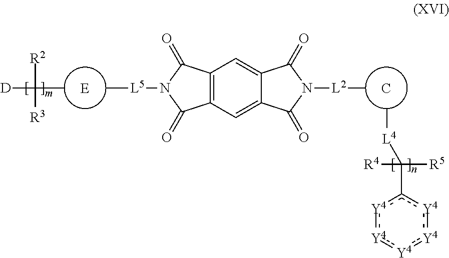

or pharmaceutically acceptable salt thereof, wherein

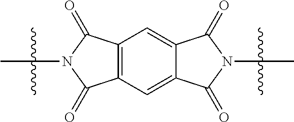 [SMILES: *n1c(=O)c2cc3c(=O)n(*)c(=O)c3cc2c1=O]

is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

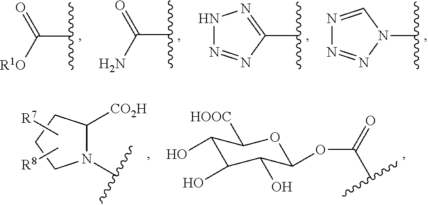

### —NR¹³SOₚR¹⁴, —C(O)—NR¹³SOₚR¹⁴,

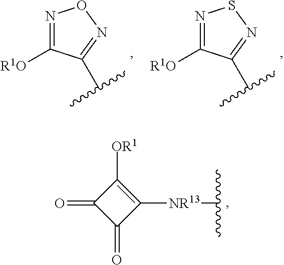

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

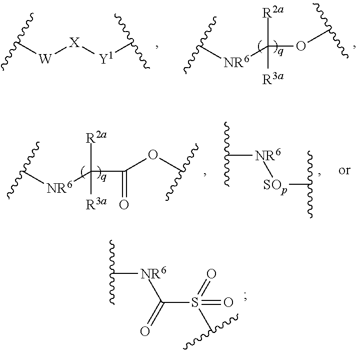

or alternatively.

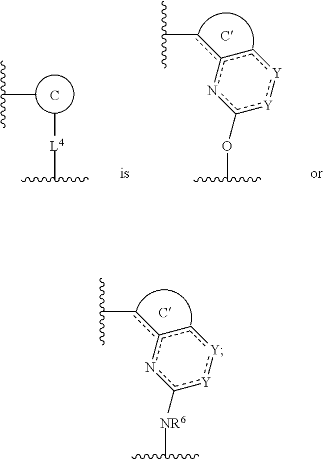

wherein

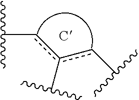

is selected from:

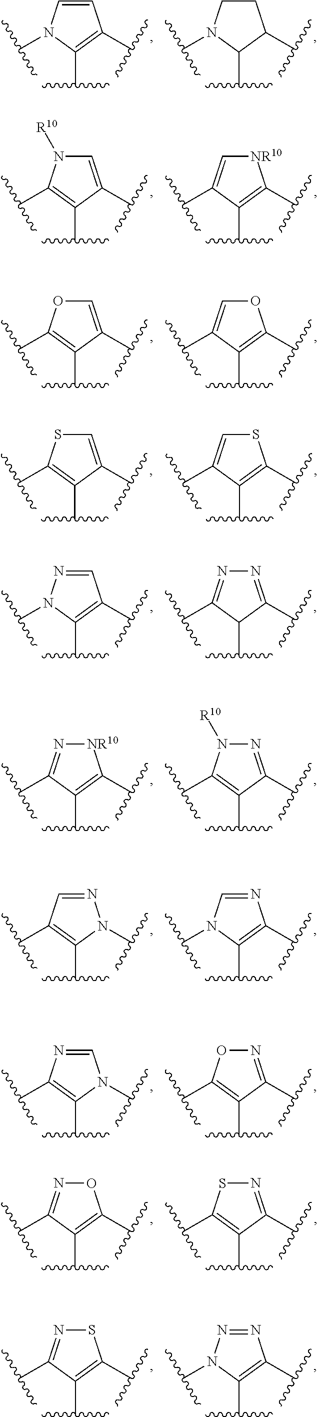

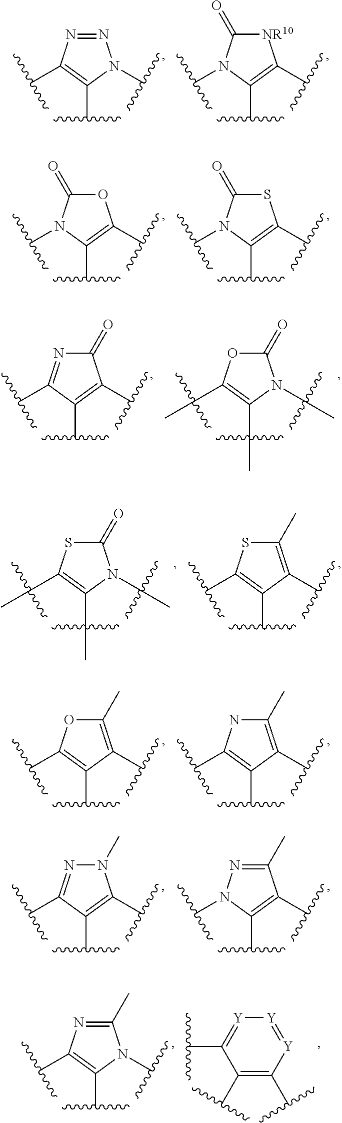

or optionally substituted variants thereof;

L² is selected from a single bond, a —CH₂— linker,

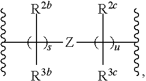

a —C≡C— linker, or a —CH═CH— linker;

L⁵ is selected from a single bond, a —CH═CH— linker, a —C≡C— linker,

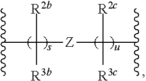

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Y⁴ is independently absent, CR⁹, C(R⁹)₂, N, or NH, provided that only one Y⁴ can be absent;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

n is an integer from 0-3;

k is an integer from 0-3;

p is an integer from 1-2;

q is an integer from 1-6;

each s and u is independently an integer from 0 to 6; and

[symbol] represents a single or double bond.

### Formula (XVII)

Some embodiments disclosed herein include a compound having the structure of Formula (XVII):

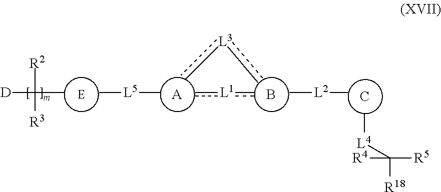

or pharmaceutically acceptable salt thereof, wherein

A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;

B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;

C is a ring system selected from the group consisting of 6-11 membered aryl, 5-11 membered heteroaryl, 5-11 membered heterocyclyl, and 5-11 membered carbocyclyl, wherein C is optionally substituted;

D is selected from —OH,

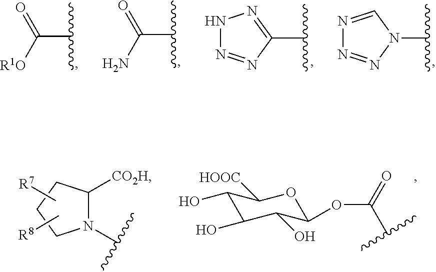

### —NR¹³SOₚR¹⁴, —C(O)—NR¹³SOₚR¹⁴,

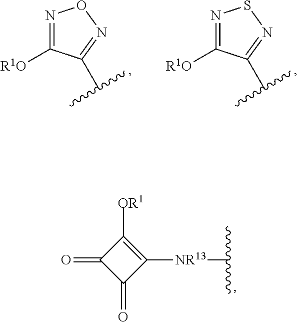

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁴ is selected from

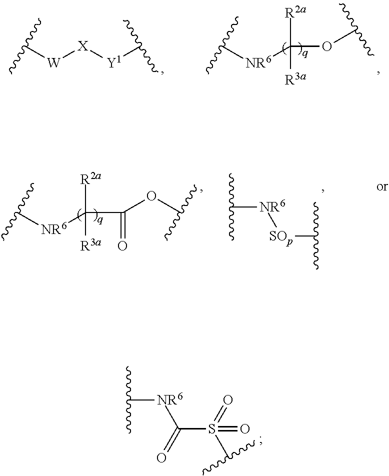

or alternatively,

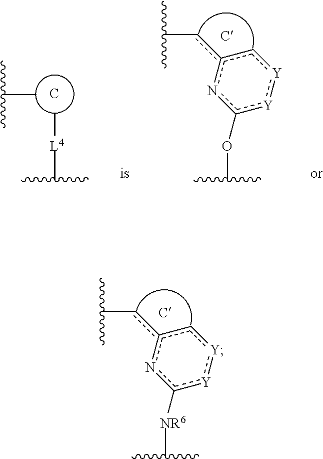

wherein

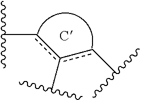

is selected from:

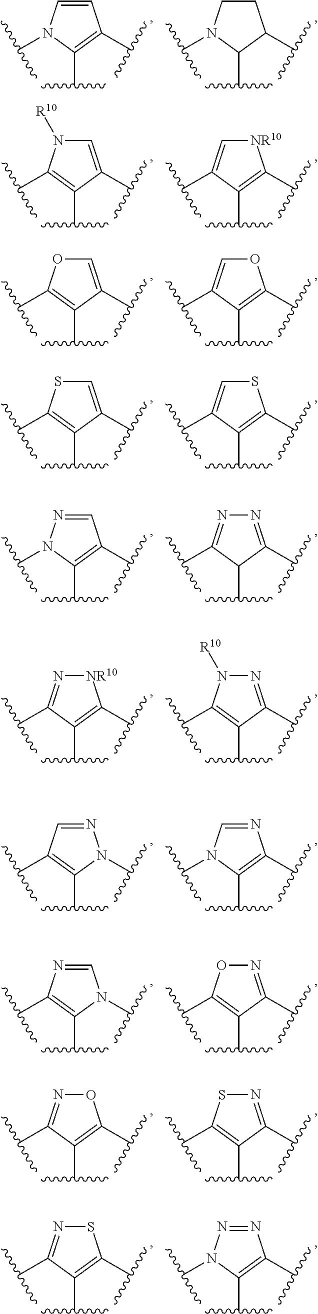

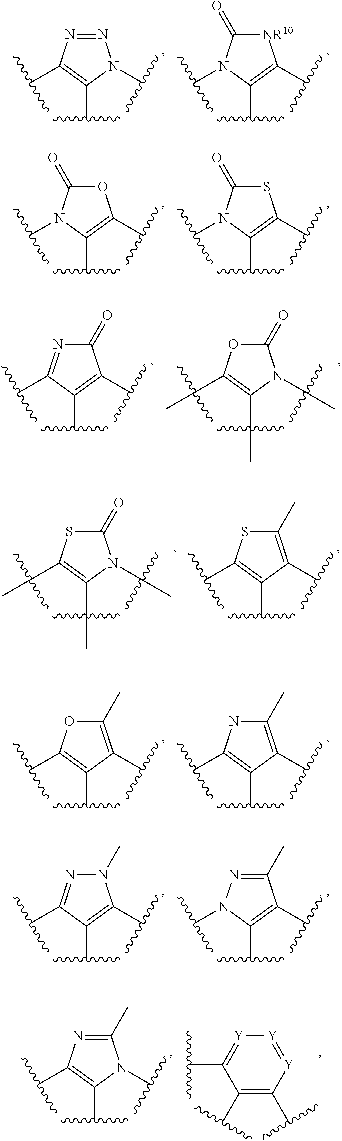

or optionally substituted variants thereof;

L¹ is selected from a single bond, a —CH₂— linker,

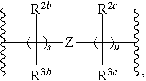

a —C≡C— linker, a —CH═CH— linker, or a ═C(R¹¹)— linker;

L² is selected from a single bond, a —CH₂— linker,

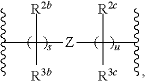

a —C≡C— linker, or a —CH═CH— linker;

L³ is absent or selected from

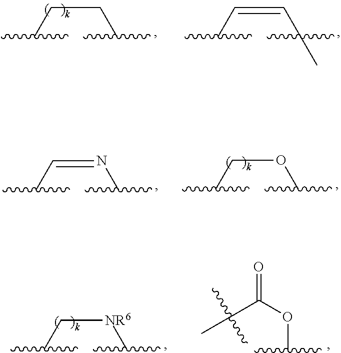

or a ═C(R¹¹)— linker;

L⁵ is selected from a single bond, a —CH═CH— linker, a —C≡C— linker,

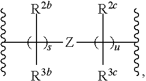

or a 4-7 membered heterocyclyl;

W is selected from C(R⁶)₂, NR⁶, or O;

X is selected from —C(O) or S(O)ₚ;

each Y is independently selected from CR⁶ or N;

Y¹ is selected from C(R⁶)₂, NR⁶, or O;

each Z is independently selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy;

each R⁶, R^{6a}, R^{6b}, and R^{6c} independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;

each R¹¹ is independently selected from hydrogen, alkyl optionally substituted with one or more substituents selected from the group consisting of halogen, hydroxy and alkoxy; halogen; haloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

R¹⁸ is C₁₋₈ alkyl, optionally substituted with one or more substituents selected from amino, halogen, hydroxy, alkoxy, haloalkyl, haloalkoxy, cyano or sulfonyl;

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

m is independently an integer from 0-3;

k is an integer from 0-3;

p is an integer from 1-2;

q is an integer from 1-6;

each s and u is independently an integer from 0 to 6; and

[symbol] represents a single or double bond.

Some embodiments disclosed herein include a pharmaceutical composition comprising an effective amount of a compound of any one of Formulae (I) through (XVII) and Tables 1 through 13, or a pharmaceutically acceptable salt thereof, and a pharmaceutically acceptable carrier, diluent, excipient or combination thereof.

Some embodiments disclosed herein include a method for treating, preventing, reversing, halting, or slowing the progression of a disease or condition selected from fibrosis, cancer, or respiratory disorders, comprising administering an effective amount of a compound of any one of Formulae (I) through (XVII) and Tables 1 through 13, a pharmaceutically acceptable salt thereof, or a pharmaceutical composition thereof to a subject in need thereof. In some embodiments, the disease or condition is fibrosis. In some embodiments, the fibrosis is selected from pulmonary fibrosis, dermal fibrosis, kidney fibrosis, or liver fibrosis. In one embodiment, the fibrosis is idiopathic pulmonary fibrosis. In some embodiments, the respiratory disorders is selected from asthma, COPD, or rhinitis. In some embodiments, the compounds described herein is administered by inhalation.

Some embodiments disclosed herein include an effective amount of a compound of any one of Formulae (I) through (XVII) and Tables 1 through 13, a pharmaceutically acceptable salt thereof, or a pharmaceutical composition thereof for use in treating, preventing, reversing, halting, or slowing the progression of a disease or condition selected from fibrosis, cancer, or respiratory disorders. In some embodiments, the disease or condition is fibrosis. In some embodiments, the fibrosis is selected from pulmonary fibrosis, dermal fibrosis, kidney fibrosis, or liver fibrosis. In one embodiment, the fibrosis is idiopathic pulmonary fibrosis. In some embodiments, the respiratory disorders is selected from asthma, COPD, or rhinitis.

Some embodiments disclosed herein include the use of an effective amount of a compound of any one of Formulae (I) through (XVII) and Tables 1 through 13, a pharmaceutically acceptable salt thereof, or a pharmaceutical composition thereof in the manufacture of a medicament for treating, preventing, reversing, halting, or slowing the progression of a disease or condition selected from fibrosis, cancer, or respiratory disorders. In some embodiments, the disease or condition is fibrosis. In some embodiments, the fibrosis is selected from pulmonary fibrosis, dermal fibrosis, kidney fibrosis, or liver fibrosis. In one embodiment, the fibrosis is idiopathic pulmonary fibrosis. In some embodiments, the respiratory disorders is selected from asthma, COPD, or rhinitis.

Some embodiments disclosed herein include a method of modulating a LPA receptor activity in a cell comprising contacting the cell with an effective amount of a compound of any one of Formulae (I) through (XVII) and Tables 1 through 13, a pharmaceutically acceptable salt thereof, or a pharmaceutical composition thereof to a subject in need thereof. In one embodiment, the LPA receptor is LPA₁.

### DETAILED DESCRIPTION OF THE PREFERRED EMBODIMENT

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The section headings used herein are for organizational purposes only and are not to be construed as limiting the subject matter described.

As used herein, common organic abbreviations are defined as follows:
Ac Acetyl
Ac₂O Acetic anhydride
aq. Aqueous
Bn Benzyl
Bz Benzoyl
BOC or Boc tert-Butoxycarbonyl
Bu n-Butyl
cat. Catalytic
Cbz Carbobenzyloxy
CDI 1,1′-carbonyldiimidazole
° C. Temperature in degrees Centigrade
DBU 1,8-Diazabicyclo[5.4.0]undec-7-ene
DCE 1,2-Dichloroethane
DCM Methylene chloride
DIEA Diisopropylethylamine
DMA Dimethylacetamide
DME Dimethoxyethane
DMF N,N′-Dimethylformamide
DMSO Dimethylsulfoxide
DPPA Diphenylphosphoryl azide
ee % Enantiomeric excess
EA Ethyl acetate
Et Ethyl
EtOAc or EA Ethyl acetate
g Gram(s)
h or hr Hour(s)
HATU 2-(1H-7-azabenzotriazol-1-yl)-1,1,3,3-tetramethyl uronium Hexafluorophosphate
HMDS hexamethyldisilazane
HOBT N-Hydroxybenzotriazole
iPr Isopropyl
LCMS Liquid chromatography-mass spectrometry
LDA Lithium diisopropylamide
LiHMDS Lithium bis(trimethylsilyl)amide
m or min Minute(s)
mCPBA meta-Chloroperoxybenzoic Acid
Me Methyl
MeOH Methanol
MeCN Acetonitrile
mL Milliliter(s)
MsCl Methanesulfonyl chloride
MTBE Methyl tertiary-butyl ether
NH₄OAc Ammonium acetate
NIS N-Iodosuccinimide
PE Petroleum ether
PG Protecting group
Pd/C Palladium on activated carbon
Pd(dppf)Cl₂ 1,1′-Bis(diphenylphosphino)ferrocene-palladium(II)dichloride
Pd(dtpbf)Cl₂ or Pd-118 [1,1′-Bis(di-tert-butylphosphino)ferrocene]dichloropalladium(II)
Ph Phenyl
ppt Precipitate
PMBC 4-Methoxybenzyl chloride
RCM Ring closing metathesis
rt Room temperature
sBuLi sec-Butylithium
SFC Supercritical fluid chromatography
TBAF Tetrabutylammonium fluoride
TEA Triethylamine
TCDI 1,1′-Thiocarbonyl diimidazole
Tert, t tertiary
TFA Trifluoroacetic acid
TFAA Trifluoroacetic acid anhydride
THF Tetrahydrofuran
TLC Thin-layer chromatography
TMEDA Tetramethylethylenediamine
TMSNCO trimethylsilyi isocyanate
μL Microliter(s)

The terms “individual,” “host,” “subject,” and “patient” are broad terms, and are to be given their ordinary and customary meaning to a person of ordinary skill in the art (and are not to be limited to a special or customized meaning), and refer without limitation to a mammal, including, but not limited to, primates, including simians (chimpanzees, apes, monkeys) and humans, cattle, horses, sheep, goats, swine, rabbits, dogs, cats, rodents, rats, mice guinea pigs, or the like.

The term “modulate” as used herein is a broad term, and is to be given its ordinary and customary meaning to a person of ordinary skill in the art (and is not to be limited to a special or customized meaning), and refers without limitation to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein is a broad term, and is to be given its ordinary and customary meaning to a person of ordinary skill in the art (and is not to be limited to a special or customized meaning), and refers without limitation to a molecule that interacts with a target either directly or indirectly. The interactions include, but are not limited to, the interactions of an agonist, partial agonist, an inverse agonist, and antagonist. In one embodiment, a modulator is an antagonist.

The term “agonist” as used herein is a broad term, and is to be given its ordinary and customary meaning to a person of ordinary skill in the art (and is not to be limited to a special or customized meaning), and refers without limitation to a molecule such as a compound, a drug, an enzyme activator or a hormone modulator that binds to a specific receptor and triggers a response in the cell. An agonist mimics the action of an endogenous ligand (such as LPA, prostaglandin, hormone, or neurotransmitter) that binds to the same receptor.

The term “antagonist” as used herein is a broad term, and is to be given its ordinary and customary meaning to a person of ordinary skill in the art (and is not to be limited to a special or customized meaning), and refers without limitation to a molecule such as a compound, which diminishes, inhibits, or prevents the action of another molecule or the activity of a receptor site. Antagonists include, but are not limited to, competitive antagonists, non-competitive antagonists, uncompetitive antagonists, partial agonists, and inverse agonists.

The term “LPA-dependent” as used herein is a broad term, and is to be given its ordinary and customary meaning to a person of ordinary skill in the art (and is not to be limited to a special or customized meaning), and refers without limitation to conditions or disorders that would not occur, or would not occur to the same extent, in the absence of LPA.

The term “LPA-mediated” as used herein is a broad term, and is to be given its ordinary and customary meaning to a person of ordinary skill in the art (and is not to be limited to a special or customized meaning), and refers without limitation to conditions or disorders that might occur in the absence of LPA but can occur in the presence of LPA.

The term “selectivity,” as applied to one LPA receptor versus other LPA receptors, as used herein is a broad term, and is to be given its ordinary and customary meaning to a person of ordinary skill in the art (and is not to be limited to a special or customized meaning), and refers without limitation to a compound that has an IC₅₀ (Ca Flux assay) for the indicated LPA receptor that is at least 10-fold less than the IC₅₀ for other LPA receptors. In some embodiments, selectivity for one LPA receptor versus other LPA receptor means that the compound has an IC₅₀ for the indicated LPA receptor that is at least 10-fold, at least 20-fold, at least 40-fold, at least 50-fold, at least 100-fold, at least 200-fold, at least 500-fold, or at least 1000-fold, less than the IC₅₀ for other LPA receptors. For example, a selective LPA₁ receptor antagonist has an IC₅₀ that is at least 10-fold, at least 20-fold, at least 40-fold, at least 50-fold, at least 100-fold, at least 200-fold, at least 500-fold, or at least 1000-fold, less than the IC₅₀ for other LPA receptors (e.g., LPA2, LPA₃).

The term “pharmaceutical combination” as used herein is a broad term, and is to be given its ordinary and customary meaning to a person of ordinary skill in the art (and is not to be limited to a special or customized meaning), and refers without limitation to a product that results from the mixing or combining of more than one active ingredient and includes both fixed and non-fixed combinations of the active ingredients. The term “fixed combination” means that the active ingredients, e.g., a compound of a preferred embodiment and a co-agent, are both administered to a patient simultaneously in the form of a single entity or dosage. The term “non-fixed combination” means that the active ingredients, e.g., a compound of a preferred embodiment and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

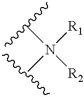

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

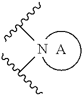

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

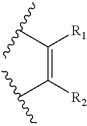

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

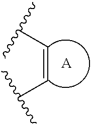

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

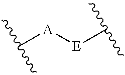

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, —CONRCN, —CH₂COOH, and —CH₂CH₂COOH, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

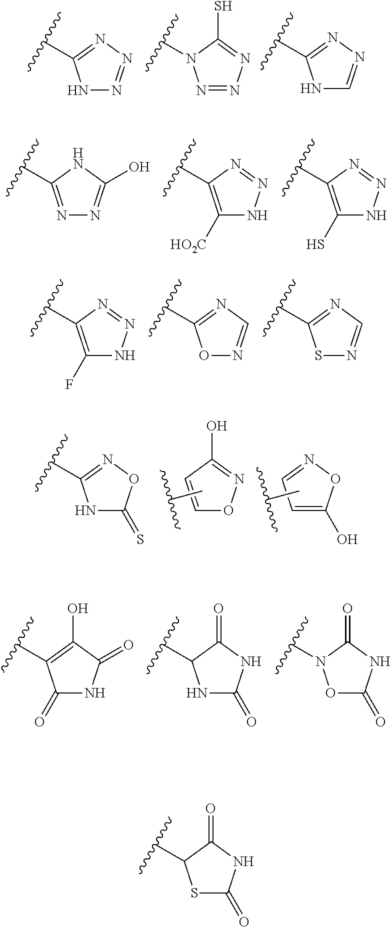

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A therapeutic effect relieves, to some extent, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Pharmaceutical salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-gluc amine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

The term “prodrug” as used herein is a broad term, and is to be given its ordinary and customary meaning to a person of ordinary skill in the art (and is not to be limited to a special or customized meaning), and refers without limitation to a compound or a pharmaceutical composition that can be administered to a patient in a less active or inactive form, which can then be metabolized in vivo into a more active metabolite. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Lysophosphatidic Acid (LPA) Activity

Lysophospholipids (such as lysophosphatidic acid (LPA)) affect fundamental cellular functions that include cellular proliferation, differentiation, survival, migration, adhesion, invasion, and morphogensis. These functions influence many biological processes that include neurogensis, angiogenesis, wound healing, immunity, and carcinogenesis. LPA acts through sets of specific G protein-coupled receptors (GPCRs) in an autocrine and paracrine fashion. LPA binding to its cognate GPCRs (LPA₁, LPA₂, LPA₃, LPA₄, LPA₅, and LPA6) activates intracellular signaling pathways to produce a variety of biological responses. LPA has a role as a biological effector molecule, and has a diverse range of physiological actions such as, but not limited to, effects on blood pressure, platelet activation, and smooth muscle contraction, and a variety of cellular effects, which include cell growth, cell rounding, neurite retraction, and actin stress fiber formation and cell migration. The effects of LPA are predominantly receptor mediated. Activation of the LPA receptors (LPA₁, LPA₂, LPA₃, LPA₄, LPA₅, and LPA6) with LPA mediates a range of downstream signaling cascades. The actual pathway and realized end point are dependent on a range of variables that include receptor usage, cell type, expression level of a receptor or signaling protein, and LPA concentration. Nearly all mammalian cells, tissues, and organs co-express several LPA-receptor subtypes, which indicates that LPA receptors signal in a cooperative manner. LPA₁, LPA₂, and LPA₃ share high amino acid sequence similarity.

A method of treatment of a preferred embodiment comprises inhibiting the physiological activity of LPA in a mammal by administering a therapeutically effective amount of a compound of a preferred embodiment or a pharmaceutically acceptable salt thereof to the mammal in need thereof.

Medicaments for treating a LPA-dependent or LPA-mediated disease or condition in a mammal are provided comprising a therapeutically effective amount of a compound of a preferred embodiment. A compound of a preferred embodiment can also be employed in the manufacture of a medicament for the treatment of a LPA-dependent or LPA-mediated disease or condition. Use of a compound of a preferred embodiment in the treatment or prevention is also provided.

In any of the methods of treatment described herein involving the treatment of LPA dependent diseases or conditions by administration of a compound of a preferred embodiment are also contemplated methods comprising administering at least one additional agent in addition to the compound of preferred embodiments. In various embodiments, each agent is administered in any order, including simultaneously. The compounds of preferred embodiments are useful as antagonists of at least one LPA receptor, or for inhibiting the activity of at least one LPA receptor, or for the treatment of a disease or condition that would benefit from inhibition of the activity of at least one LPA receptor.

The compounds of preferred embodiments, pharmaceutically acceptable salts, pharmaceutically acceptable prodrugs, and pharmaceutically acceptable solvates thereof, which are antagonists of at least one LPA receptor (e.g., LPA₁, LPA₂, LPA₃) can be used to treat patients suffering from one or more LPA-dependent or LPA-mediated conditions or diseases, including, but not limited to, ideopathic pulmonary fibrosis. In some embodiments, LPA-dependent conditions or diseases include those wherein an absolute or relative excess of LPA is present and/or observed.

One or more of the compounds of preferred embodiments can be provided in the form of pharmaceutically acceptable salts, solvates, active metabolites, tautomers, or prodrugs thereof. The compounds of preferred embodiments can be provided in pharmaceutical compositions comprising a therapeutically effective amount of the compound. In some embodiments, the pharmaceutical composition also contains at least one pharmaceutically acceptable inactive ingredient. The pharmaceutical composition can be formulated for intravenous injection, subcutaneous injection, oral administration, inhalation, nasal administration, topical administration, ophthalmic administration, or otic administration. The pharmaceutical composition can be in the form of a tablet, a pill, a capsule, a liquid, an inhalant, a nasal spray solution, a suppository, a suspension, a gel, a colloid, a dispersion, a solution, an emulsion, an ointment, a lotion, an eye drop, or an ear drop.

The pharmaceutical compositions of preferred embodiments can further comprise one or more additional therapeutically active agents other than a compound of the preferred embodiments. Such agents can include, but are not limited to, corticosteroids, immunosuppresants, analgesics, anti-cancer agent, anti-inflammatories, chemokine receptor antagonists, bronchodilators, leukotriene receptor antagonists, leukotriene formation inhibitors, monoacylglycerol kinase inhibitors, phospholipase A₁ inhibitors, phospholipase A₂ inhibitors, and lysophospholipase D (lysoPLD) inhibitors, autotaxin inhibitors, decongestants, antihistamines, mucolytics, anticholinergics, antitussives, expectorants, and β13-2 agonists.

Other objects, features, and advantages of the compounds, methods, and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description

### Compounds

Formula (I)

Some embodiments disclosed herein include a compound of Formula (I) as described above or a pharmaceutically acceptable salt thereof.

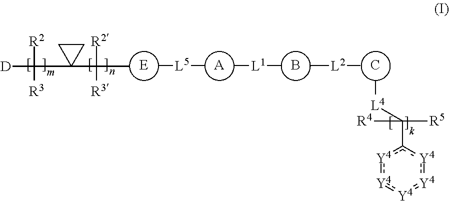

In some embodiments, one of A or B is an acetylene and the other one of A or B is selected from the group consisting of:

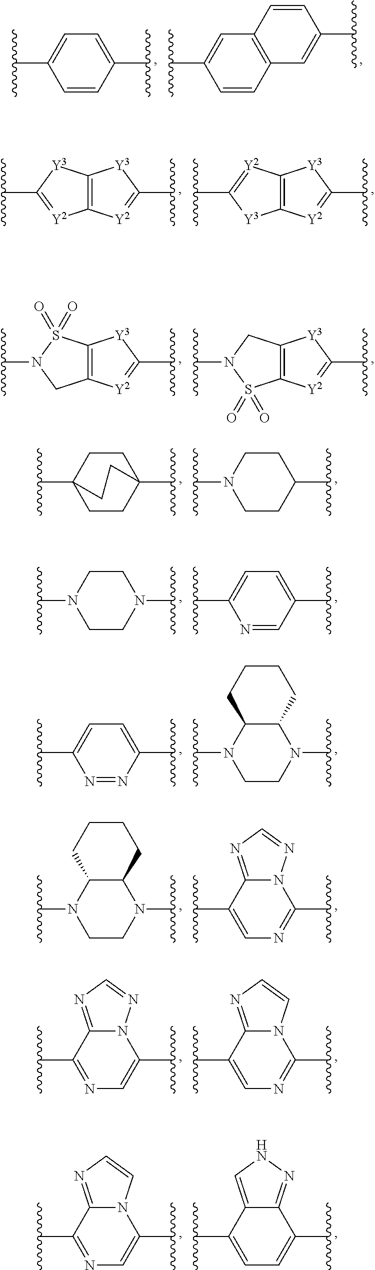

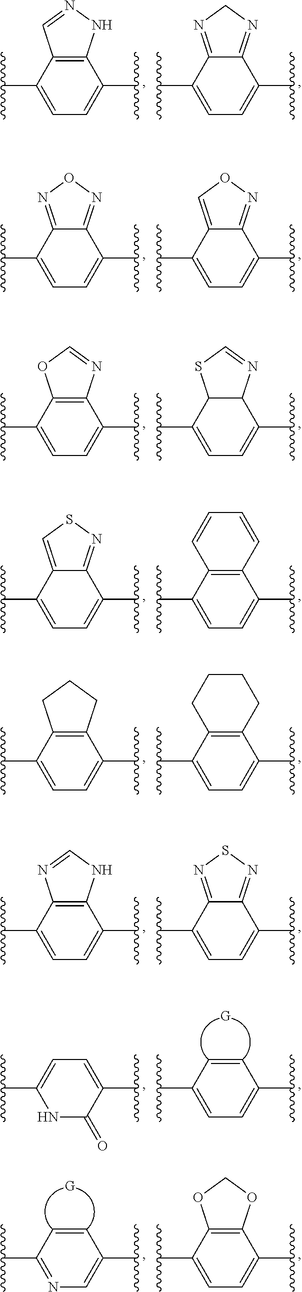

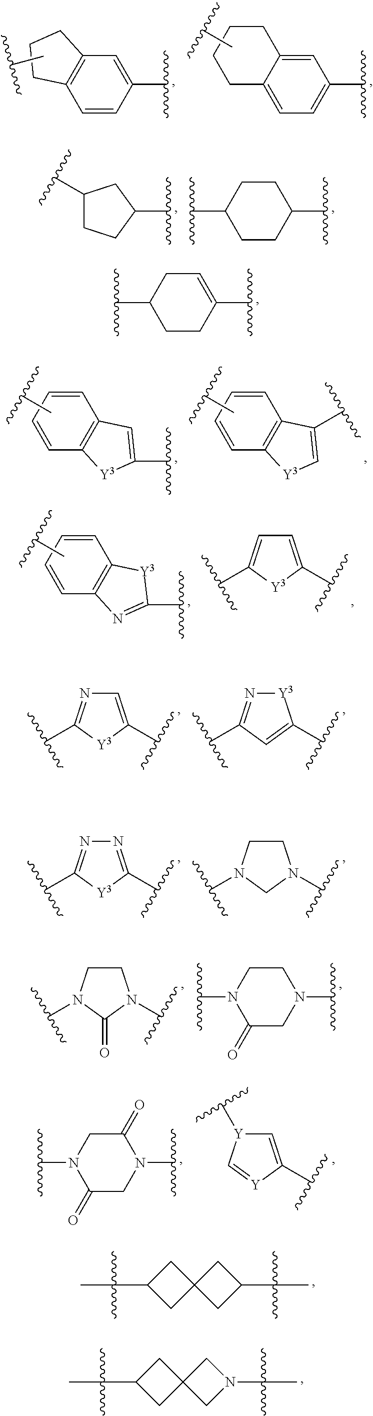

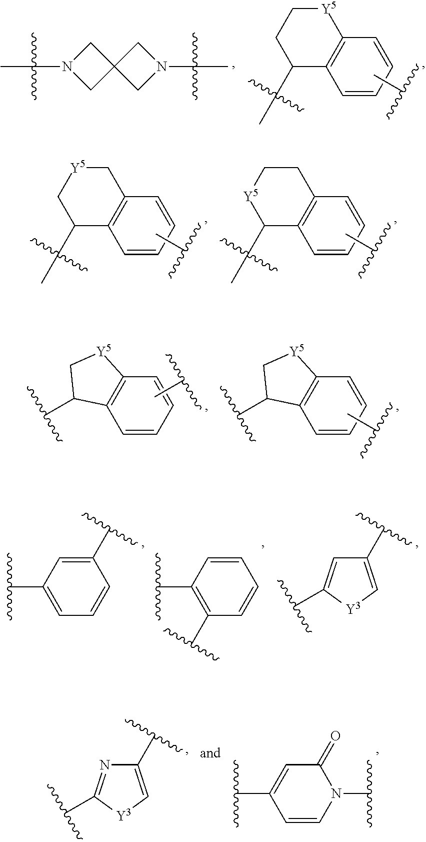

wherein the rings in A or B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

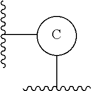

is selected from

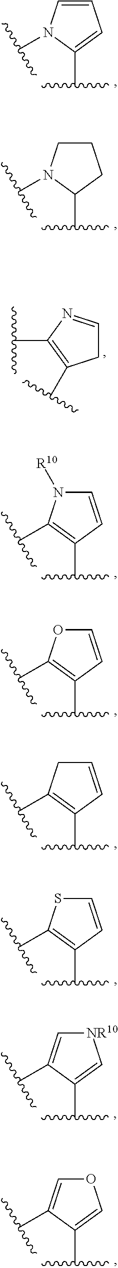

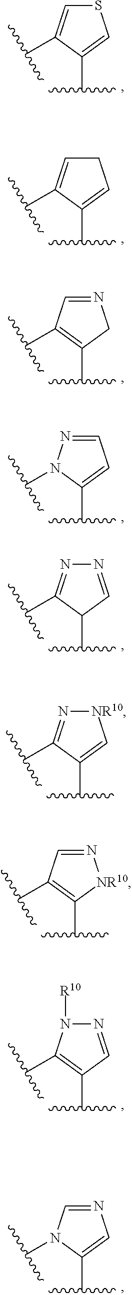

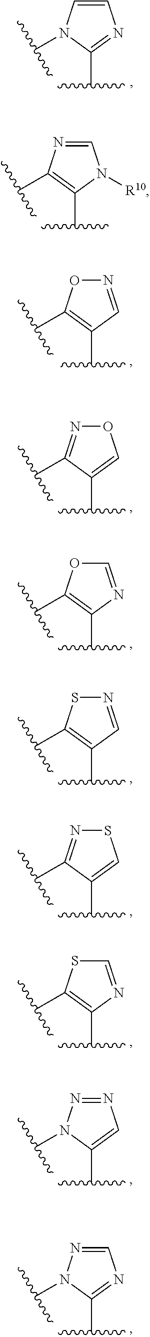

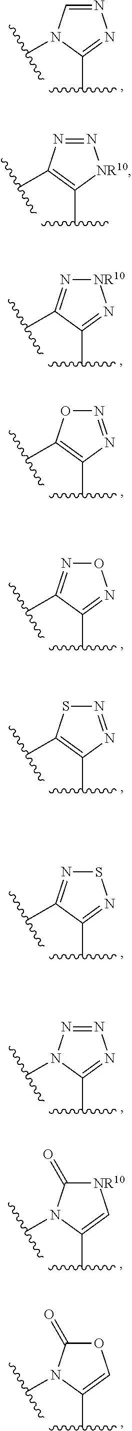

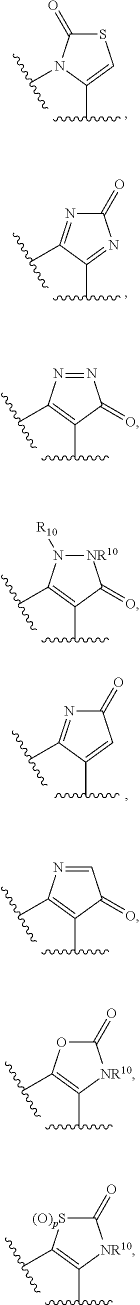

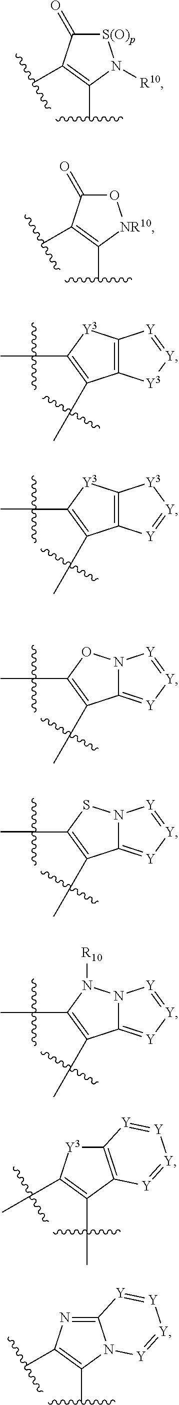

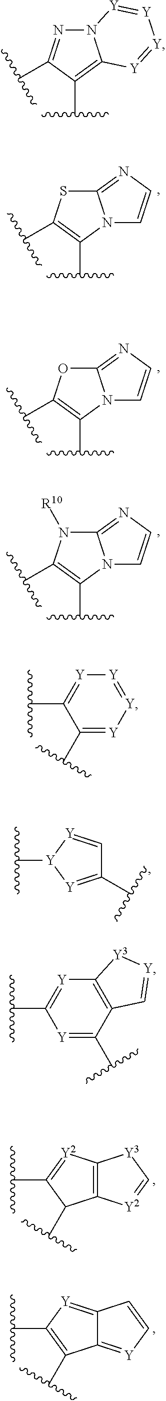

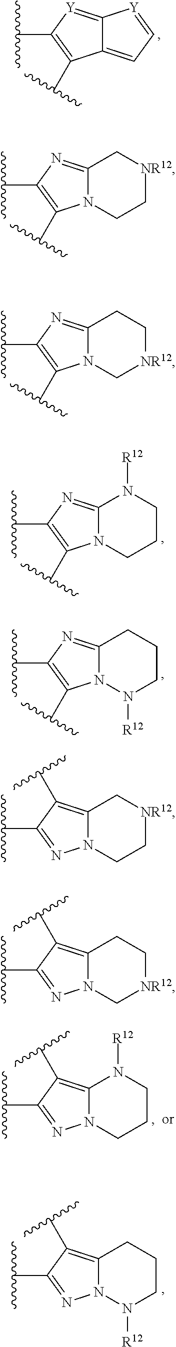

R¹², or optionally substituted variants thereof; each Y² is independently selected from —CH═ or N; each Y³ is independently selected from C(R⁶)₂, NR⁶, O or S; each Y⁵ is independently selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (I) is also represented by Formula (Ia):

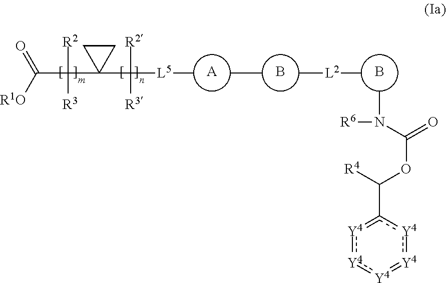

wherein L² and L⁵ are each independently selected from a single bond, a —CH₂O— linker, or a —CH═CH— linker; and R⁴ is selected from hydrogen or alkyl optionally substituted with halogen.

In some embodiments, the ring system in each of A, B and G is unsubstituted or substituted with one or more substituents selected from alkyl, haloalkyl, halogen, hydroxy, alkoxy, haloalkoxy, cyano, or oxo; D is selected from

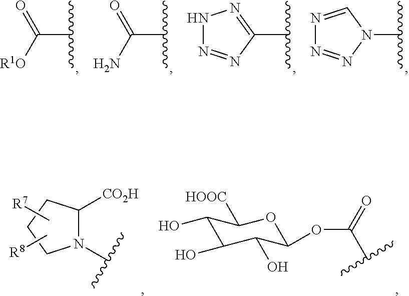

or carboxylic acid isosteres; E is absent; L⁴ is selected from

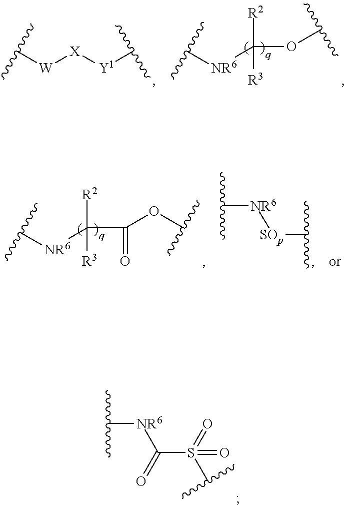

L¹ is selected from selected from a single bond, a —O— linker, a

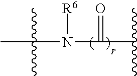

linker, a —C(O)— linker, a —CH₂— linker, a —CH₂O— linker, a —C≡C— linker, or a —CH═CH— linker; L² is selected from a single bond, a —O— linker, a

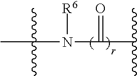

linker, a —C(O)— linker, a —CH₂— linker, a —CH₂O— linker, a —C≡C— linker, or a —CH═CH— linker; L⁵ is selected from a single bond, a —CH₂O— linker, a —CH═CH— linker, a —C≡C— linker, a

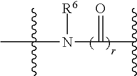

linker, or a 4-7 membered heterocyclyl;

R¹ is selected from hydrogen or alkyl; R², R³, R^{2′}, and R^{3′} are each independently selected from hydrogen, alkyl, aryl, or heteroaryl; or R² and R³ are joined together with the atom to which they are attached to form an optionally substituted cycloalkyl or an optionally substituted heterocyclyl; or R^{2′} and R^{3′} are joined together with the atom to which they are attached to form an optionally substituted cycloalkyl or an optionally substituted heterocyclyl; or R^{2′} is selected from hydrogen, alkyl, aryl, or heteroaryl and R^{3′} is joined to an atom alpha to a point of attachment of L⁵ to A to form an optionally substituted cycloalkyl or an optionally substituted heterocyclyl; or R^{3′} is selected from hydrogen, alkyl, aryl or heteroaryl and R^{2′} is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments, B is an acetylene and A is selected from

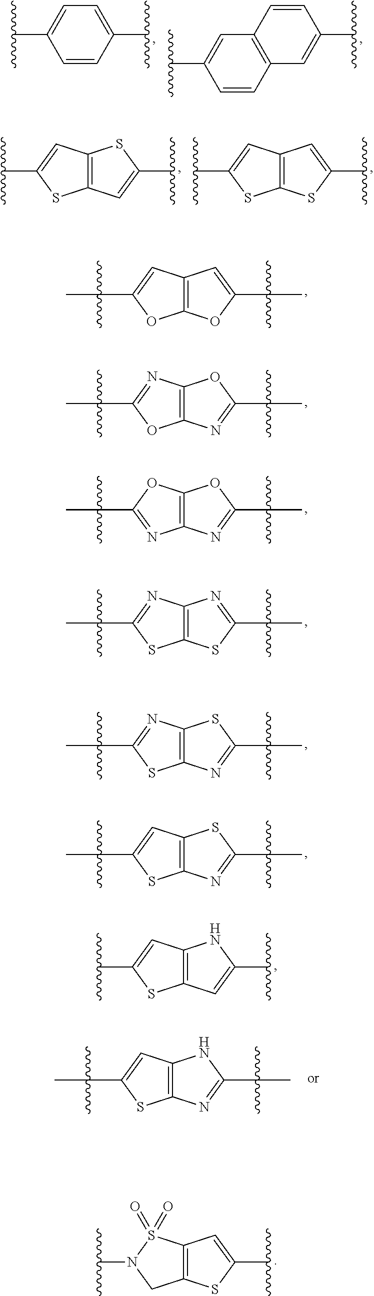

In one embodiment, A is phenyl. In another embodiment, A is naphthyl.

In some embodiments, A is an acetylene and B is selected from

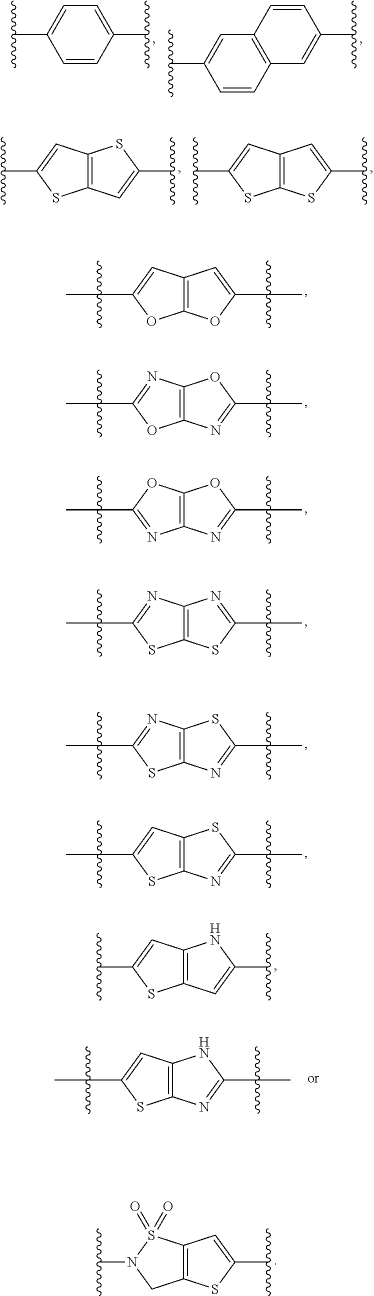

In one embodiment, B is phenyl. In another embodiment, B is naphthyl.

In some embodiments described herein of the compounds of Formula (I) or (Ia), rings in A are unsubstituted.

In some embodiments described herein of the compounds of Formula (I) or (Ia), rings in B are unsubstituted.

In some embodiments described herein of the compounds of Formula (I) or (Ia), rings in A can be optionally substituted. In some such embodiments, A can be substituted with one or more substituents selected from alkyl, amino, haloalkyl, halogen, hydroxy, alkoxy, haloalkoxy, cyano, or oxo. In some embodiments, A can be substituted with one or more sulfonyl, for example, methanesulfonyl.

In some embodiments described herein of the compounds of Formula (I) or (Ia), rings in B can be optionally substituted. In some such embodiments, B can be substituted with one or more substituents selected from alkyl, amino, haloalkyl, halogen, hydroxy, alkoxy, haloalkoxy, cyano, or oxo. In some embodiments, B can be substituted with one or more sulfonyl, for example, methanesulfonyl.

In some embodiments described herein of the compounds of Formula (I), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments described herein of the compounds of Formula (I), E can be unsubstituted or substituted. In some embodiments, E can be substituted with with one or more substituents selected from alkyl, amino, haloalkyl, halogen, hydroxy, alkoxy, haloalkoxy, cyano, or oxo. In some embodiments, E can be substituted with one or more sulfonyl, for example, methanesulfonyl.

In some embodiments, m is 0 and n is 1. In some other embodiments, m is 1 and n is 0

In some embodiments, R⁶ is hydrogen. In some embodiments, R¹ is hydrogen.

In some embodiments, each of R², R³, R^{2′} and R^{3′} is hydrogen.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

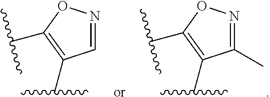

In some embodiments, C is selected from

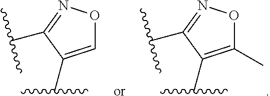

In some embodiments, C is selected from

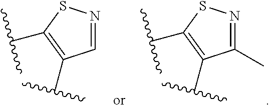

In some embodiments, C is selected from

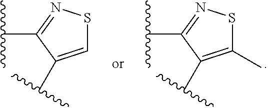

In some embodiments, C is selected from

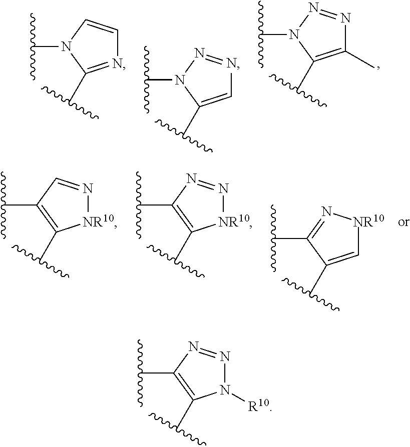

In some such embodiments, R¹⁰ is selected from C₁₋₃ alkyl or C₃₋₆ cycloalkyl. In some such embodiments, R¹⁰ is hydrogen.

In some embodiments, C is selected from

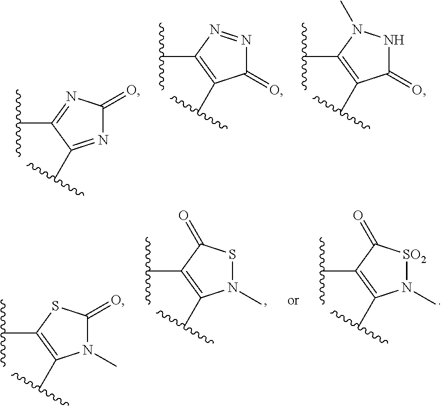

In some embodiments, C is selected from

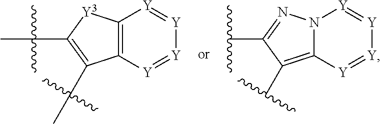

wherein Y³ is selected from O or S. In some such embodiments, each Y is a CR⁶. In some other such embodiments, at least one Y is nitrogen. In some embodiments, C is optionally substituted

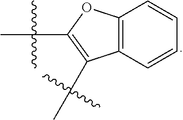 [SMILES: *c1oc2ccccc2c1*]

In some embodiments, L² is a single bond. In some embodiments, L⁵ is a single bond.

In some embodiments of the compound of Formula (I) or (Ia),

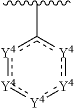

can be

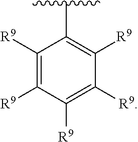

In some such embodiments, each of R⁹ is hydrogen. In some other such embodiments, at least one R⁹ is selected from C₁₋₃ alkyl or halogen.

In some embodiments,

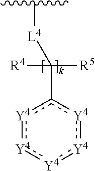

can be

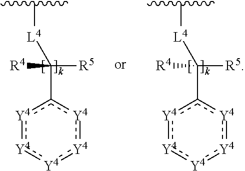

Some embodiments of the compounds of Formula (I) are selected from compounds of Table 1 as shown below, and pharmaceutically acceptable salts thereof.

Some embodiments of the compounds of Formula (I) are selected from compounds IT001, IT002, IT003 or IT065, as shown in Table 13.

Formula (II)

Some embodiments disclosed herein include a compound of Formula (II) as described above or a pharmaceutically acceptable salt thereof.

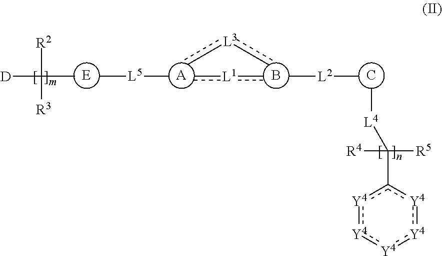

In some embodiments, each of A and B can be an acetylene or selected from the group consisting of:

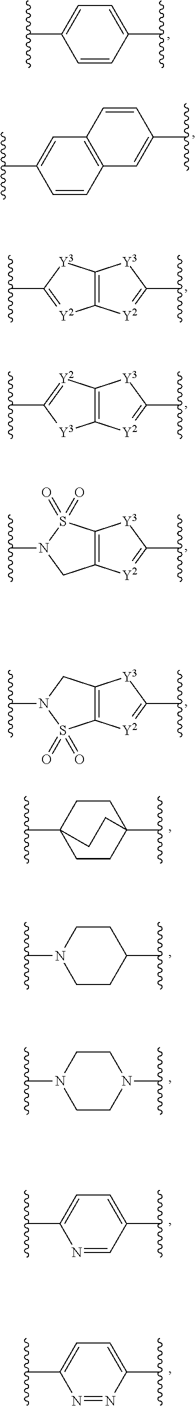

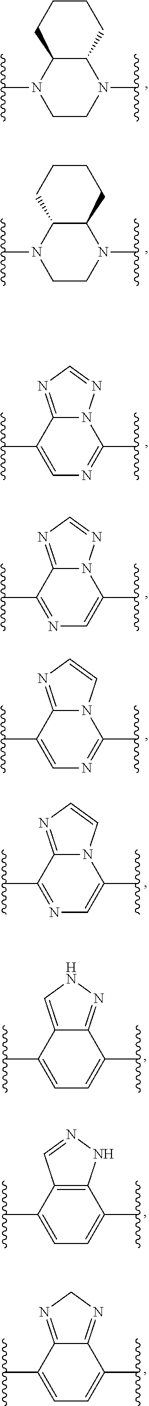

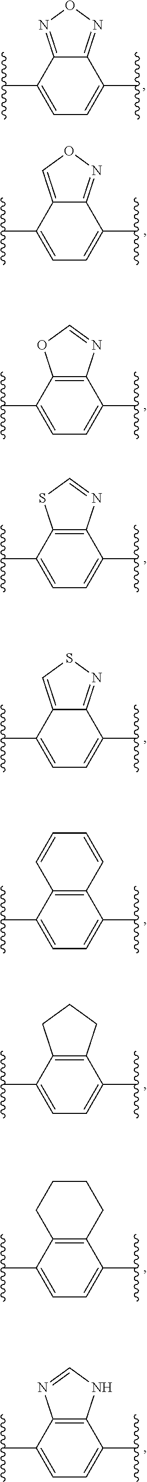

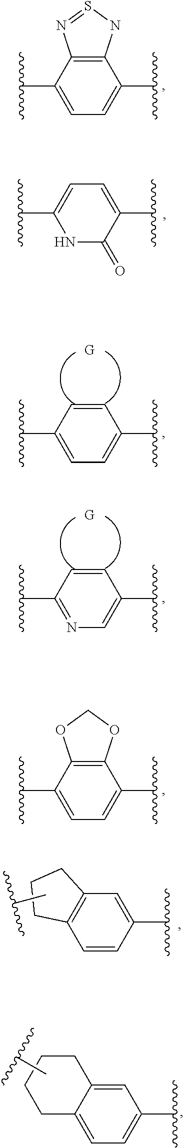

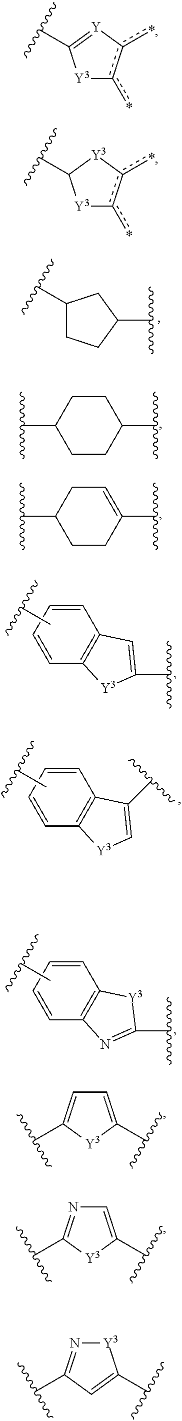

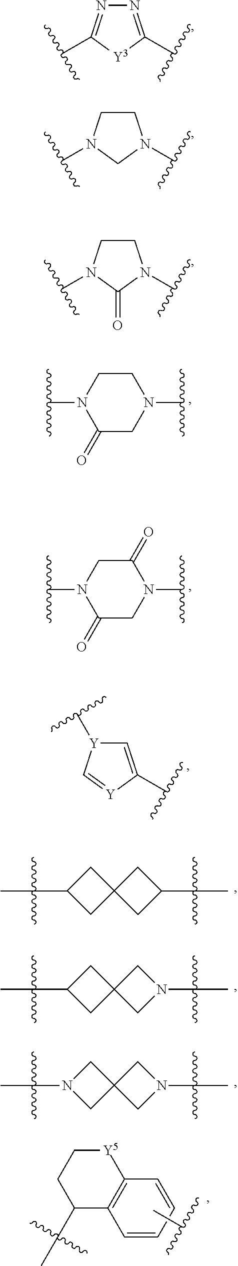

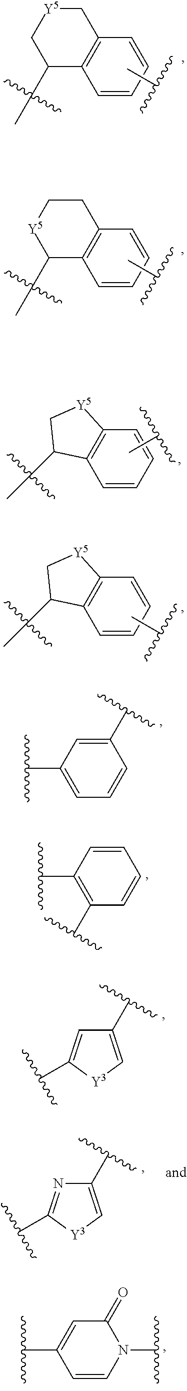

wherein each * is a point of attachment of A or B to L¹ or L³, and wherein the rings in A and/or B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, or oxo;

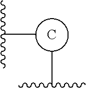

is selected from

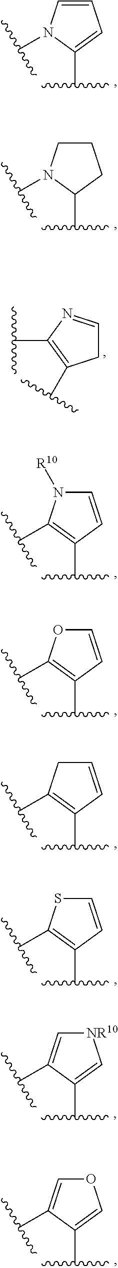

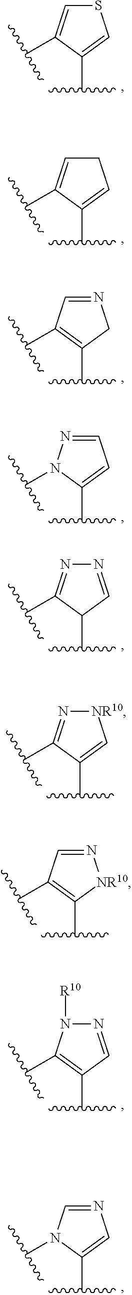

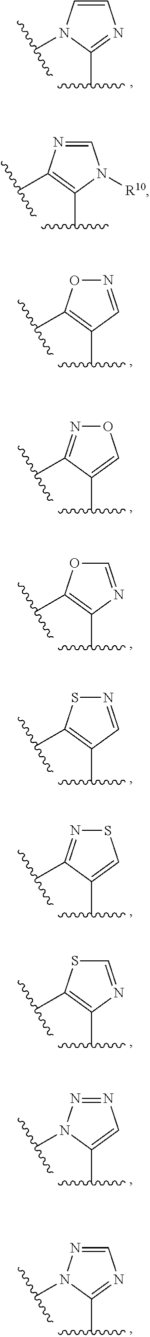

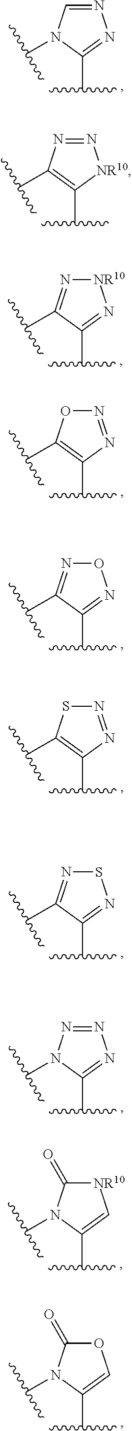

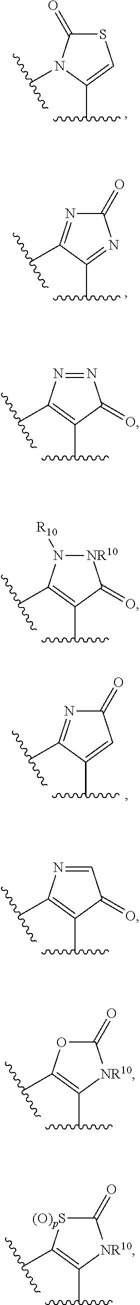

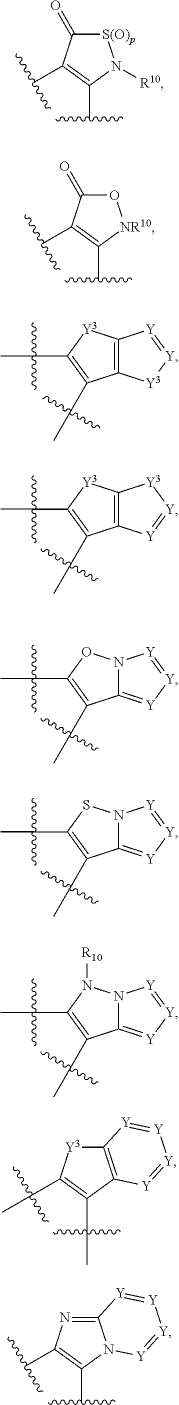

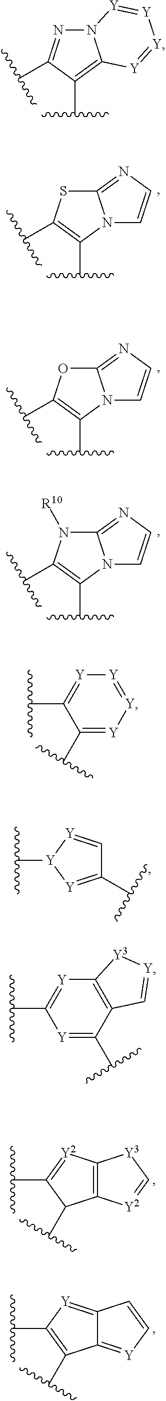

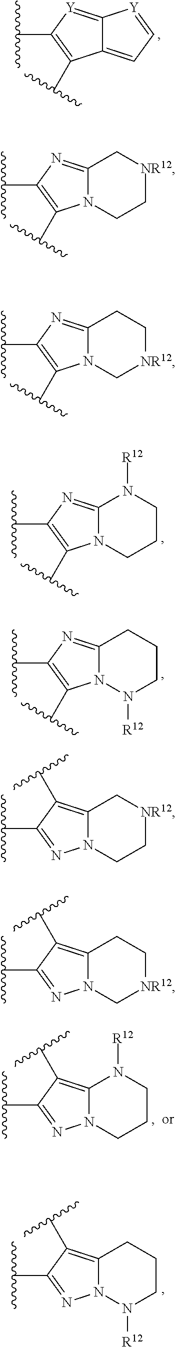

or optionally substituted variants thereof; each Y² is independently selected from —CH═ or N; each Y³ is independently selected from C(R⁶)₂, NR⁶, O or S; each Y⁵ is independently selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (II) is also represented by Formula (IIa):

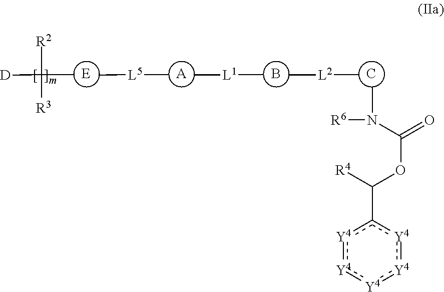

wherein A is selected from acetylene,

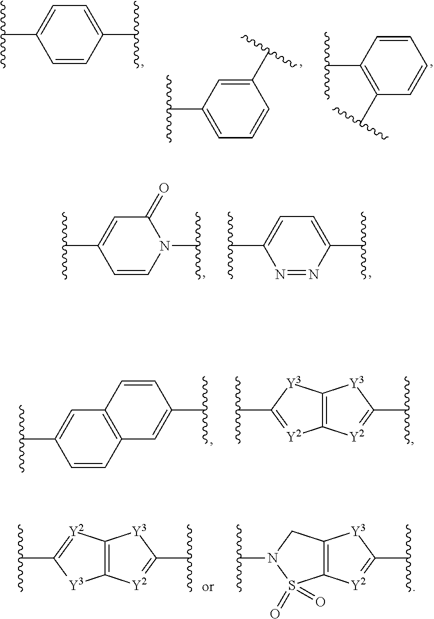

B is selected from acetylene,

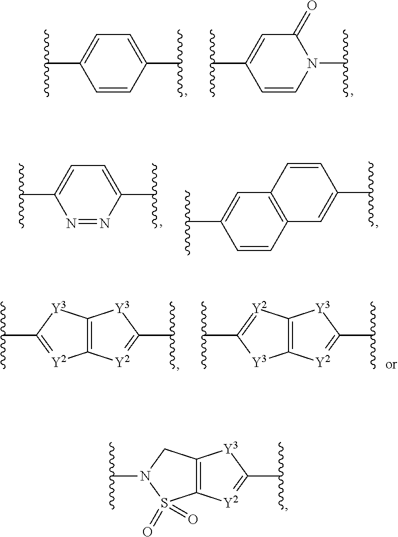

wherein the rings in A and/or B are unsubstituted or substituted with one or more substituents selected from alkyl, amino, haloalkyl, halogen, hydroxy, alkoxy, haloalkoxy, cyano, or oxo; L¹ is selected from a single bond, a —C(O)— linker, a —CH₂— linker, or a —CH₂O— linker; L² is selected from a single bond, a —O— linker, a —NH— linker, a —C(O)— linker, a —CH₂— linker, or a —CH₂O— linker; and R⁴ is selected from hydrogen or alkyl optionally substituted with halogen.

In some embodiments, each of the rings in A, B and G is unsubstituted or substituted with one or more substituents selected from alkyl, halogen, haloalkyl, hydroxy, alkoxy, haloalkoxy, cyano, or oxo; D is selected from

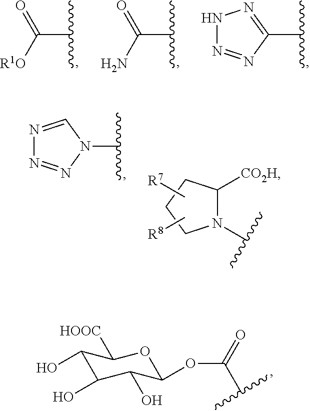

or carboxylic acid isosteres; L⁴ is selected from

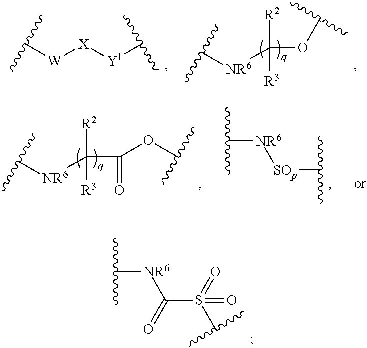

L¹ is selected from a single bond, a —O— linker, a

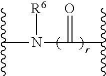

linker, a —C(O)— linker, a —CH₂— linker, a —CH₂O— linker, a —C≡C— linker, a —CH═CH— linker, or a ═C(R¹¹)— linker; L² is selected from a single bond, a —O— linker, a

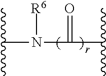

linker, a —C(O)— linker, a —CH₂— linker, a —CH₂O— linker, a —C≡C— linker, or a —CH═CH— linker; L⁵ is selected from

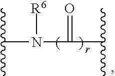

or a —C≡C— linker; E is absent;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments, the compound of Formula (II) is also represented by Formula (IIb):

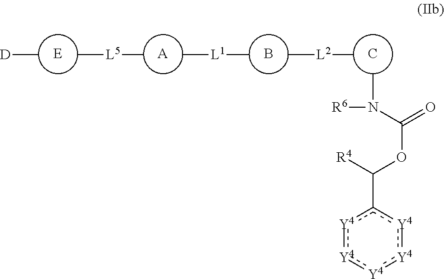

wherein L⁵ is

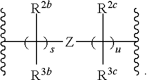

In some such embodiments, L⁵ is selected from a —O— linker, a —S— linker, a —NH— linker, a —NH—C(O)— linker or a —SO₂— linker.

In some embodiments, the compound of Formula (II) is also represented by Formula (IIc):

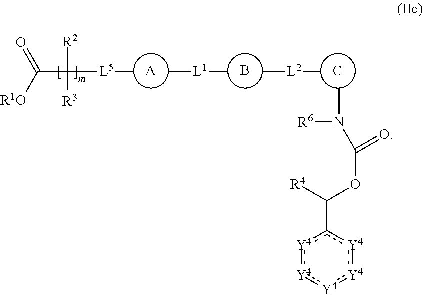

In some embodiments, E is absent.

In some embodiments, E is a phenylene, optionally substituted with one or more substituents selected from alkyl, amino, halogen, haloalkyl, hydroxy, alkoxy, haloalkoxy, cyano, or oxo. In some such embodiments, E is an optionally substituted

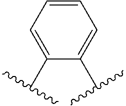 [SMILES: *c1ccccc1*]

or optionally substituted

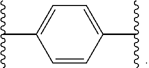 [SMILES: *c1ccc(*)cc1]

In some such embodiments, E is an optionally substituted

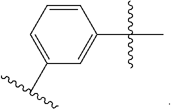 [SMILES: *c1cccc(*)c1]

In some embodiments, each of

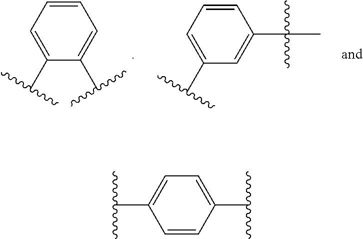 [SMILES: *c1ccccc1* ; *c1cccc(*)c1 ; *c1ccc(*)cc1]

is optionally substituted with one or more halogens. In some embodiments, the optionally substituted halogen is fluoro.

In some embodiments, E is a six-membered heteroarylene comprising one or two nitrogen atoms, wherein E is optionally substituted with one or more substituents selected from alkyl, amino, halogen, haloalkyl, hydroxy, alkoxy, haloalkoxy, cyano, or oxo. In some such embodiments, E is selected from

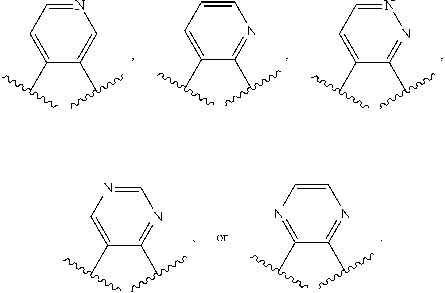 [SMILES: *c1ccncc1* ; *c1cccnc1* ; *c1ccnnc1* ; *c1cncnc1* ; *c1nccnc1*]

In some embodiments, E is a five to ten membered heteroarylene comprising one to three heteroatoms selected from nitrogen, oxygen or sulfur, wherein E is optionally substituted with one or more substituents selected from alkyl, amino, halogen, haloalkyl, hydroxy, alkoxy, haloalkoxy, cyano, or oxo. In some such embodiments, E is selected from

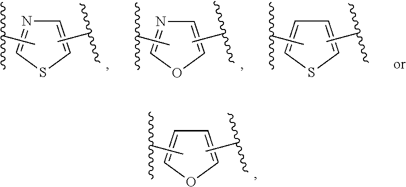

and in some other embodiments, E is selected from

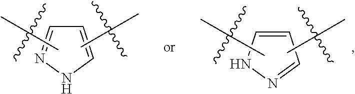

In some such embodiments, E is selected from

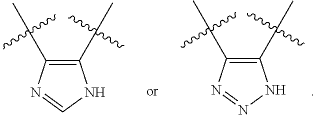 [SMILES: *c1nc[nH]c1* ; *c1nn[nH]c1*]

In some such embodiments, E is selected from

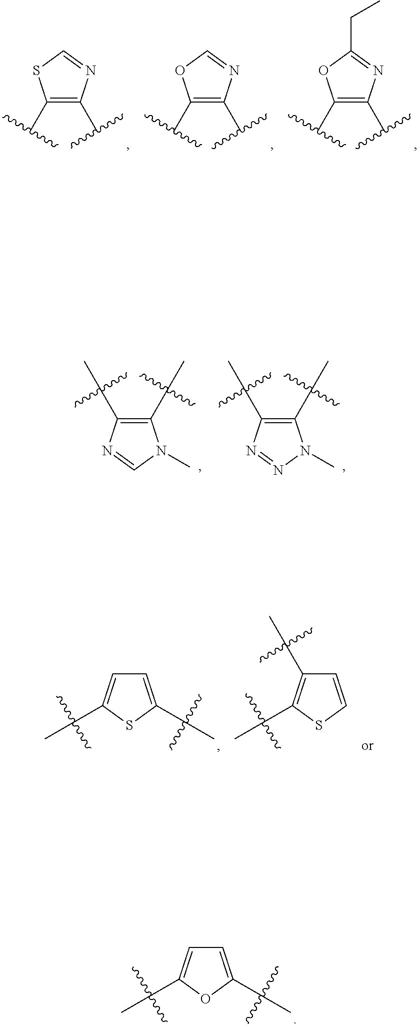

In some such embodiments, E is selected from

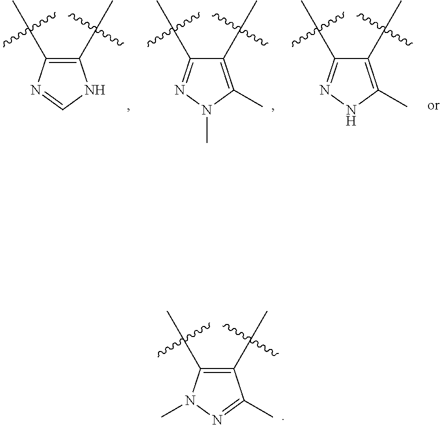

In some such embodiments, E is selected from

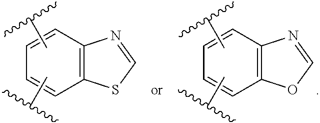

In some such embodiments, E is selected from

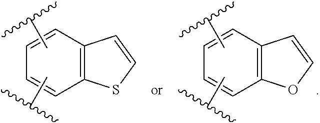

In some of these embodiments, E is selected from

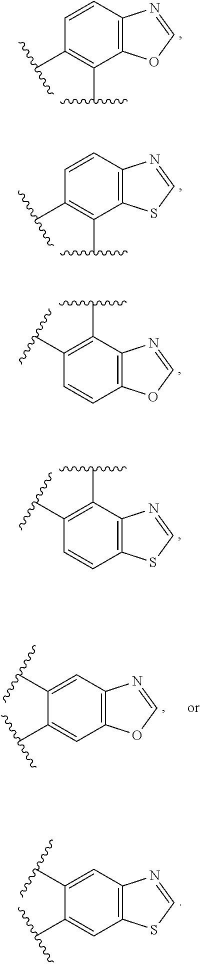 [SMILES: *c1ccc2ncoc2c1* ; *c1ccc2ncsc2c1* ; *c1ccc2ocnc2c1* ; *c1ccc2scnc2c1* ; *c1cc2ncoc2cc1* ; *c1cc2ncsc2cc1*]

In some of these embodiments, E is selected from

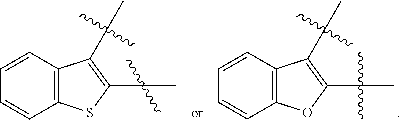 [SMILES: *c1sc2ccccc2c1* ; *c1oc2ccccc2c1*]

In some embodiments, E is selected from

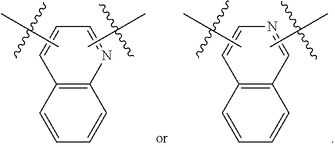

In some of these embodiments, E is selected from

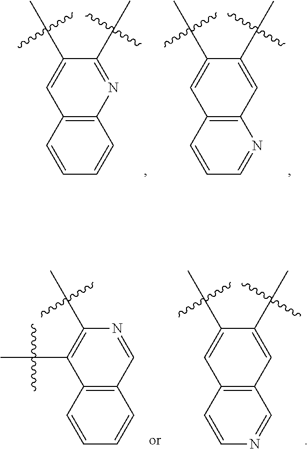 [SMILES: *c1cc2ccccc2nc1* ; *c1cc2cccnc2cc1* ; *c1ncc2ccccc2c1* ; *c1cc2ccncc2cc1*]

In any of the embodiments of E described herein, E can be each optionally substituted with one or more substituents selected from alkyl, amino, halogen, haloalkyl, hydroxy, alkoxy, haloalkoxy, cyano, or oxo.

In some embodiments, A is

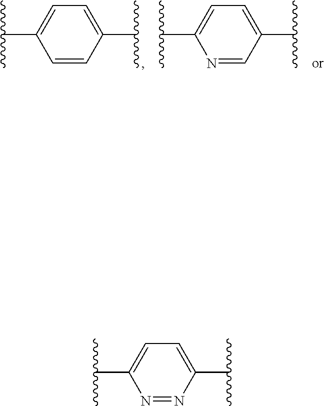

and B is selected from acetylene,

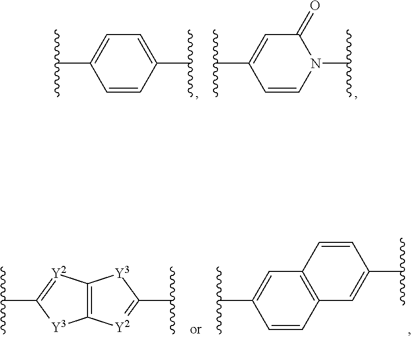

each can be optionally substituted with one or more substituents selected from alkyl, amino, halogen, haloalkyl, hydroxy, alkoxy, haloalkoxy, cyano, or oxo. In some such embodiments, A is

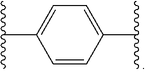 [SMILES: *c1ccc(*)cc1]

In some such embodiments, A is

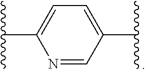 [SMILES: *c1ccc(*)nc1]

In some such embodiments, A is

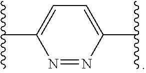 [SMILES: *c1ccc(*)nn1]

In some such embodiments, B is

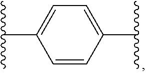 [SMILES: *c1ccc(*)cc1]

optionally substituted with one or more halogens. In some such embodiments, B is acetylene. In some such embodiments, B is

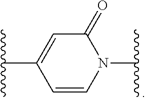 [SMILES: *c1ccn(*)c(=O)c1]

In some such embodiments, B is

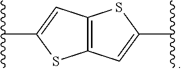 [SMILES: *c1cc2sc(*)cc2s1]

In some these embodiments, the rings in each A and/or B can be optionally substituted.

In some embodiments, A is

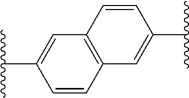 [SMILES: *c1ccc2cc(*)ccc2c1]

and B is selected from acetylene or

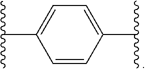 [SMILES: *c1ccc(*)cc1]

In one such embodiment, B is acetylene.

In some embodiments, A is

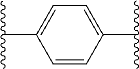 [SMILES: *c1ccc(*)cc1]

and B is selected from acetylene,

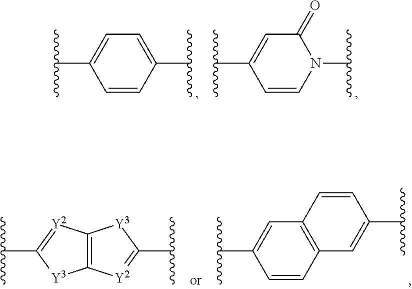

each A and B can be optionally substituted. In some such embodiments, A is optionally substituted with one or more halogens. In some further embodiments, A is optionally substituted with one or more fluoro. In some such embodiments, B is acetylene. In some such embodiments, B is

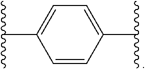 [SMILES: *c1ccc(*)cc1]

In some such embodiments, B is

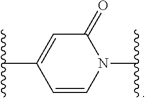 [SMILES: *c1ccn(*)c(=O)c1]

In some such embodiments, B is

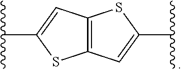 [SMILES: *c1cc2sc(*)cc2s1]

In some embodiments, B is

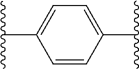 [SMILES: *c1ccc(*)cc1]

and A is selected from acetylene,

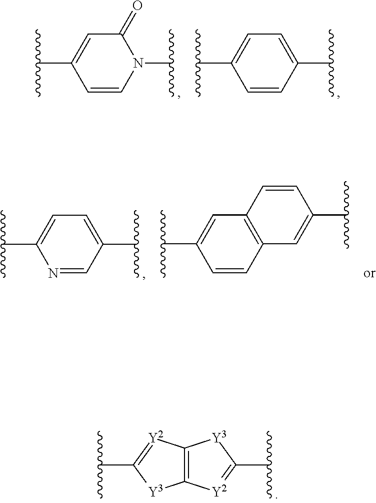

In some of these embodiments, the rings in each A and/or B can be optionally substituted, for example, optionally substituted with one or more substituents selected from alkyl, amino, haloalkyl, halogen, hydroxy, alkoxy, haloalkoxy, cyano, or oxo. In some such embodiments, A is acetylene. In some such embodiments, A is

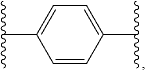 [SMILES: *c1ccc(*)cc1]

optionally substituted with one or more halogens. In some further such embodiments, A is substituted with one or more fluoro. In some such embodiments, A is

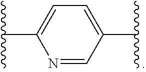 [SMILES: *c1ccc(*)nc1]

In some such embodiments, A is

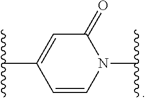 [SMILES: *c1ccn(*)c(=O)c1]

In some such embodiments, A is

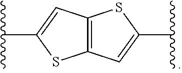 [SMILES: *c1cc2sc(*)cc2s1]

In some other embodiments, B is

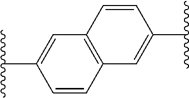 [SMILES: *c1ccc2cc(*)ccc2c1]

and A is selected from acetylene or

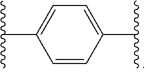 [SMILES: *c1ccc(*)cc1]

In one such embodiment, A is acetylene.

In some embodiments described herein of the compounds of Formula (II), (IIa), (IIb) or (IIc), rings in A are unsubstituted.

In some embodiments described herein of the compounds of Formula (II), (IIa), (IIb) or (IIc), rings in B are unsubstituted.

In some embodiments described herein of the compounds of Formula (II), (IIa), (IIb) or (IIc), rings in A can be substituted. In some such embodiments, A can be substituted with one or more substituents selected from alkyl, amino, haloalkyl, halogen, hydroxy, alkoxy, haloalkoxy, cyano, or oxo. In some embodiments, A can be substituted with one or more sulfonyl, for example, methanesulfonyl.

In some embodiments described herein of the compounds of Formula (II), (IIa), (IIb) or (IIc), rings in B can be substituted. In some such embodiments, B can be substituted with one or more substituents selected from alkyl, amino, haloalkyl, halogen, hydroxy, alkoxy, haloalkoxy, cyano, sulfonyl or oxo. In some embodiments, B can be substituted with one or more sulfonyl, for example, methanesulfonyl.

In some embodiments described herein of the compounds of Formula (II), (IIa) or (IIb), E can be absent. In some embodiments described herein of the compounds of Formula (II), (IIa) or (IIb), E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

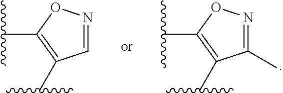

In some embodiments, C is selected from

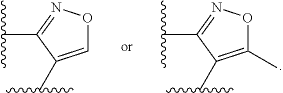

In some embodiments, C is selected from

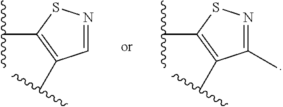

In some embodiments, C is selected from

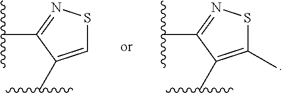

In some embodiments, C is selected from

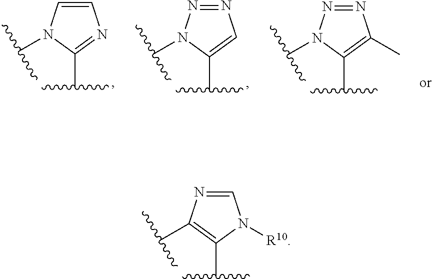

In some embodiments, C is

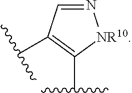

In some embodiments, C is

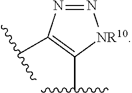

In some embodiments, C is

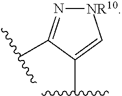

In some such embodiments, R¹⁰ is selected from C₁₋₃ alkyl or C₃₋₆ cycloalkyl. In some such embodiments, R¹⁰ is hydrogen. In some such embodiments, C is selected from

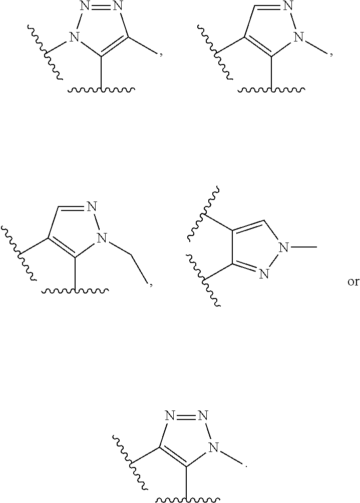

In some embodiments, C is selected from

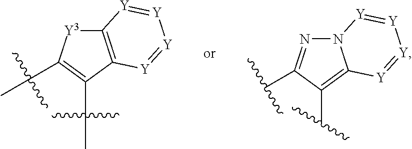

wherein Y³ is selected from O or S. In some such embodiments, Y is a CR⁶. In some other such embodiments, at least one Y is nitrogen. In some embodiments, C is optionally substituted

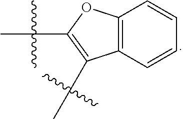 [SMILES: *c1oc2ccccc2c1*]

In some embodiments, C is selected from

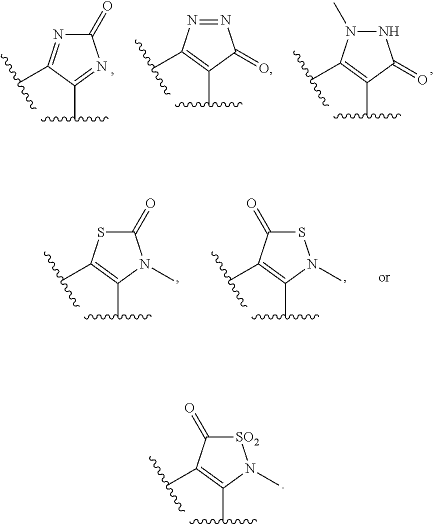

In some embodiments, m is 0. In some other embodiments, m is 1. In some other embodiments, m is 2.

In some embodiments, each of R² and R³ is hydrogen. In some embodiments, at least one of R² and R³ is alkyl, aryl or halogen.

In some embodiments, both R² and R³ are alkyl. In some such embodiments, both R² and R³ are methyl.

In some embodiments, one of R² or R³ is alkyl and the other R² or R³ is halogen. In some such embodiments, one of R² or R³ is methyl and the other R² or R³ is fluoro.

In some embodiments, both R² and R³ are halogens. In some such embodiments, both R² and R³ are fluoro.

In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl. In another embodiment, R² and R³ are joined together with the atom to which they are attached to form an optionally substituted cyclobutyl. In yet antoher embodiment, R² and R³ are joined together with the atom to which they are attached to form an optionally substituted cyclopentyl. In yet another embodiment, R² and R³ are joined together with the atom to which they are attached to form an optionally substituted oxetane.

In some embodiments, R⁶ is hydrogen.

In some embodiments, L¹ is a single bond. In some embodiments, L² is a single bond.

In some embodiment, L⁵ is

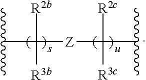

In some such embodiments, one of s or u in L⁵ is 0. In some such embodiments, both s and u in L⁵ are 0. In some such embodiments, L⁵ is —NH—. In some such embodiments, L⁵ is —C(O)—NH—. In some such embodiments, L⁵ is —O—. In some such embodiments, L⁵ is —S—. In some such embodiments, L⁵ is —SO₂—. In some embodiments, L⁵ is —C≡C—.

In some embodiments, D is —C(O)OR¹. In some such embodiments, R¹ is hydrogen. In some such embodiments, R¹ is alkyl.

Some embodiments of the compounds of Formula (II), (IIa), (IIb) or (IIc),

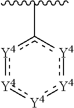

can be

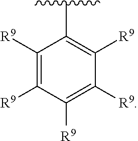

In some such embodiments, each of R⁹ is hydrogen. In some other such embodiments, at least one R⁹ is selected from C₁₋₃ alkyl or halogen.

In some embodiments,

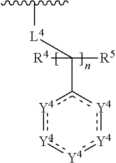

can be

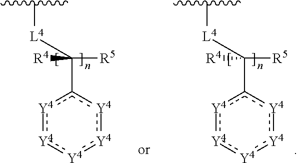

Some embodiments of the compounds of Formula (II) are selected from compounds of Table 2, Table 2A, Table 2B, Table 2C and Table 2D as shown below, and pharmaceutically acceptable salt thereof.

Some embodiments of the compounds of Formula (II) are selected from the group consisting of IT005, IT006, IT155, IT194-IT199, IT226-IT232, IT238, IT256-259, IT277, IT300, IT301, IT303-IT316, IT344, IT345, IT355, IT356, IT368, IT374, IT375, IT388, IT398-IT409, IT417, IT419, IT420, IT423-IT425, IT428-IT432, IT434-IT440, IT444, IT446-IT457, IT459-IT474, IT476-IT478, IT481-IT492, IT495, IT497, or IT500-IT514 as shown in Table 13.

Formula (III)

Some embodiments disclosed herein include a compound of Formula (III) as described above or a pharmaceutically acceptable salt thereof.

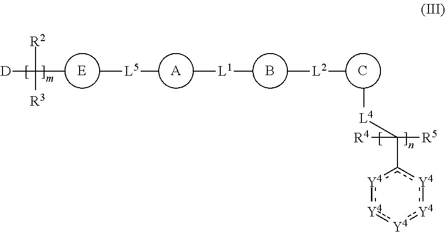

In some embodiments, one of A or B is selected from the group consisting of

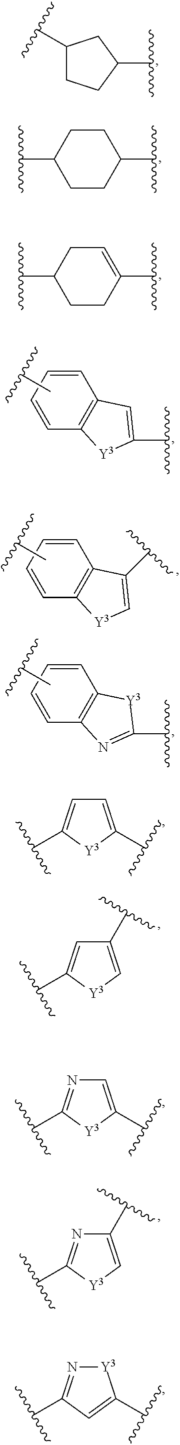

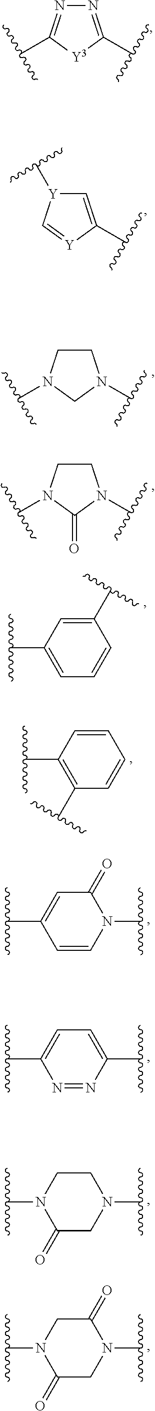

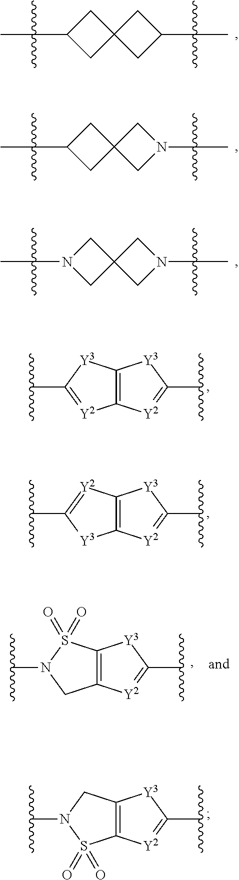

and the other one of A or B is selected from

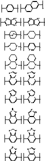

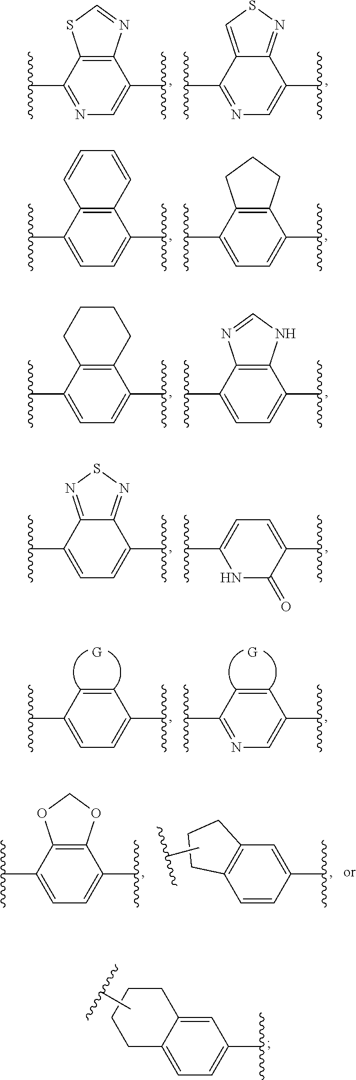

wherein each of A and B is unsubstituted or substituted with one or more substituents selected from alkyl, amino, halogen, haloalkyl, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, or oxo;

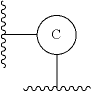

is selected from

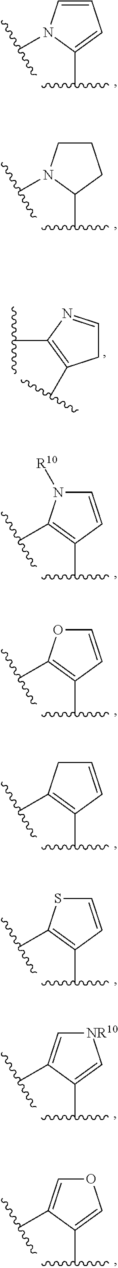

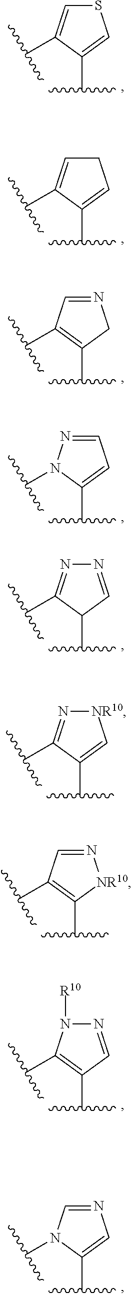

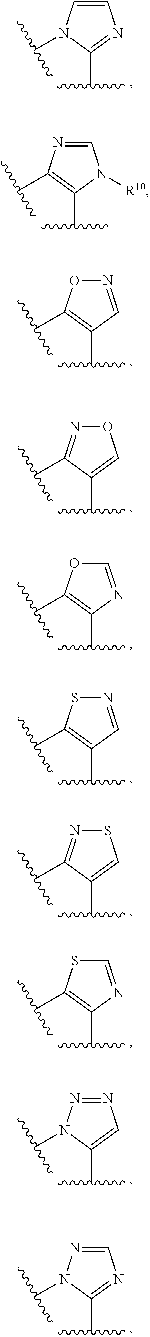

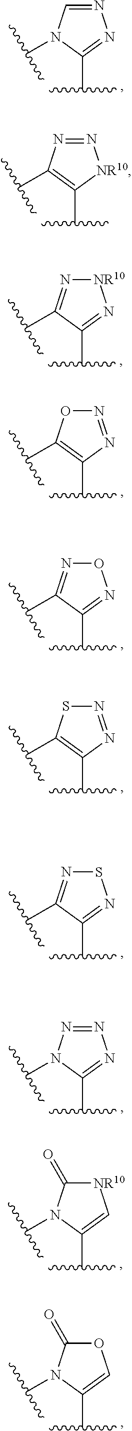

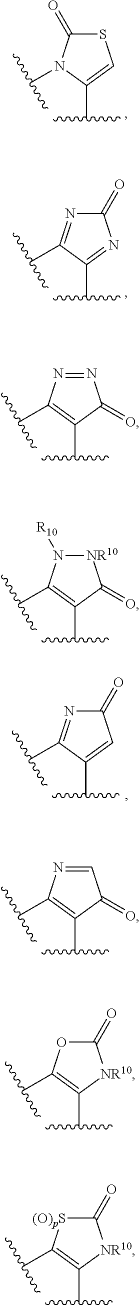

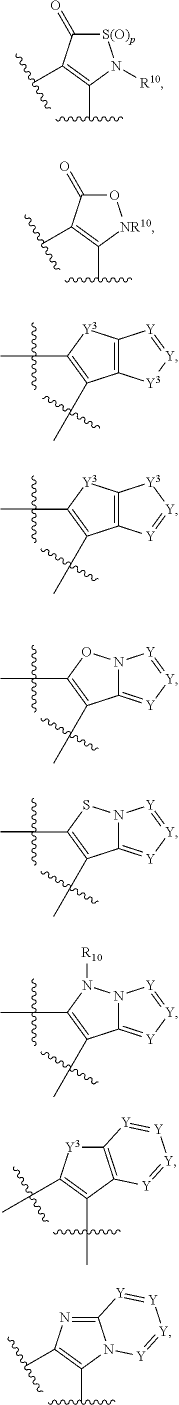

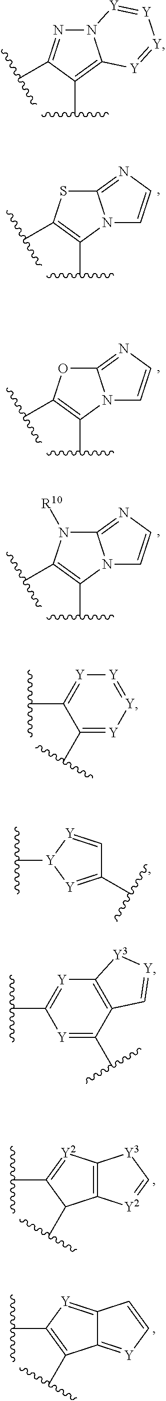

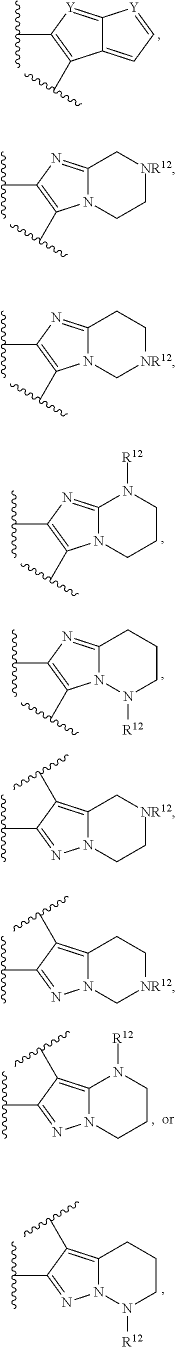

or optionally substituted variants thereof; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, one of A or B is selected from

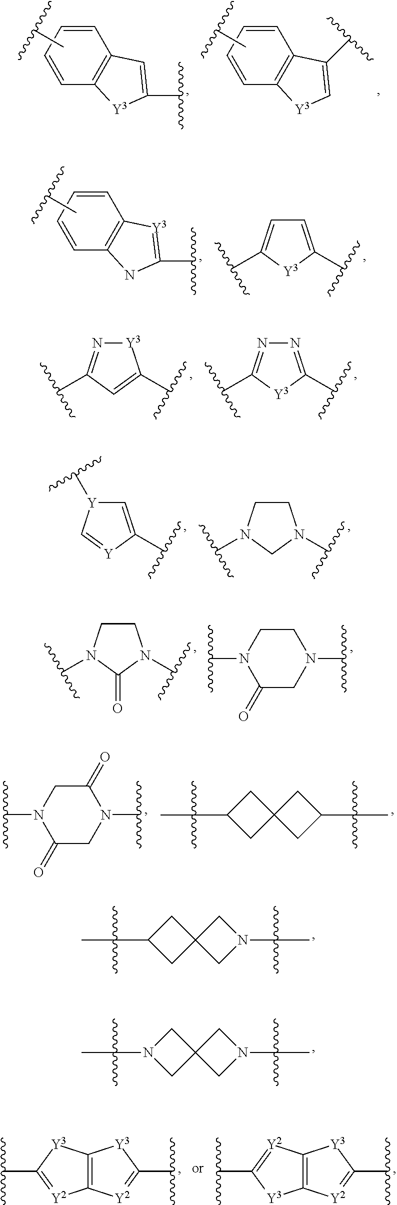

and the other one of A or B is selected from

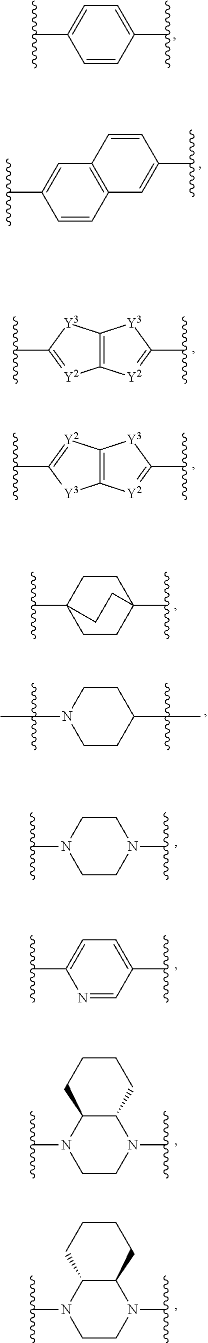

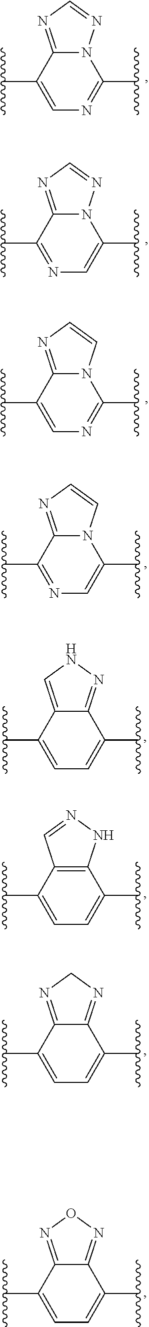

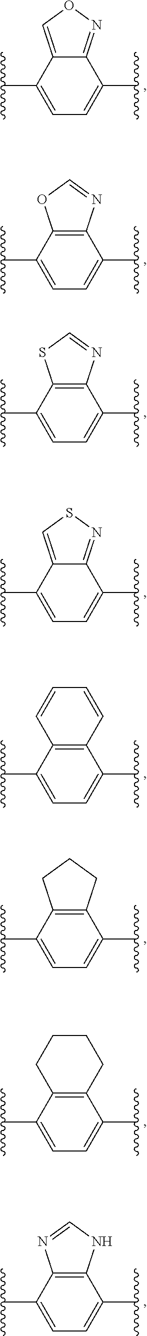

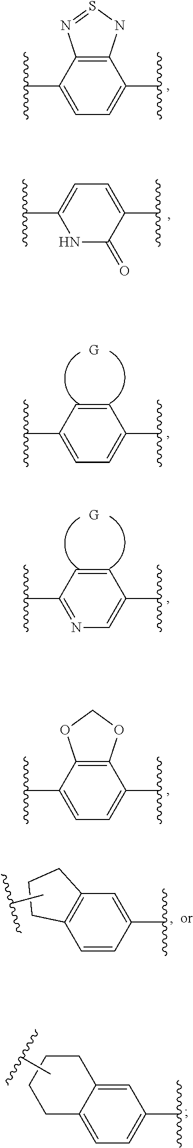

wherein each of A and B is unsubstituted or substituted with one or more substituents selected from alkyl, amino, halogen, haloalkyl, hydroxy, alkoxy, haloalkoxy, cyano, or oxo.

In some embodiments, the compound of Formula (III) is also represented by Formula (IIIa):

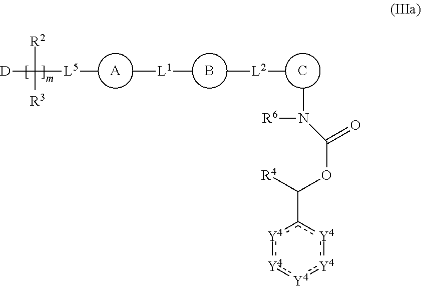

wherein A is selected from

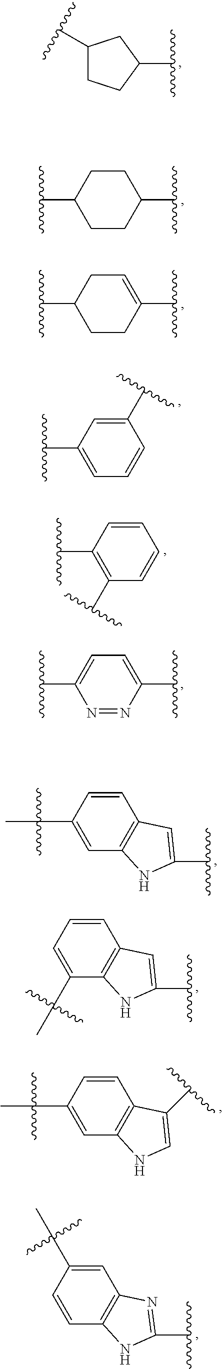

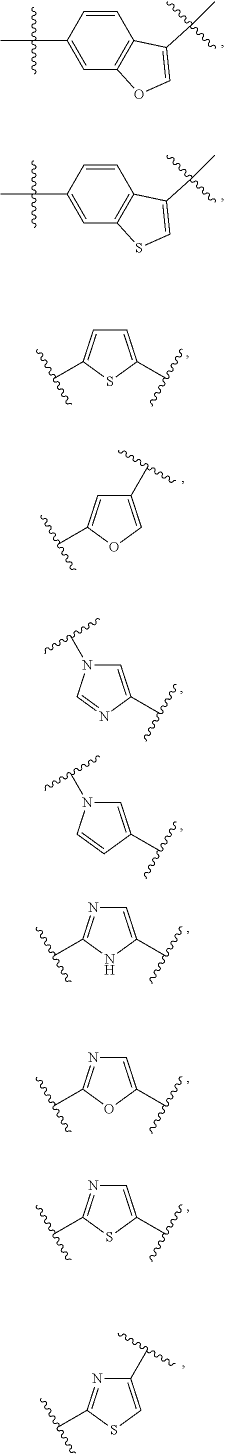

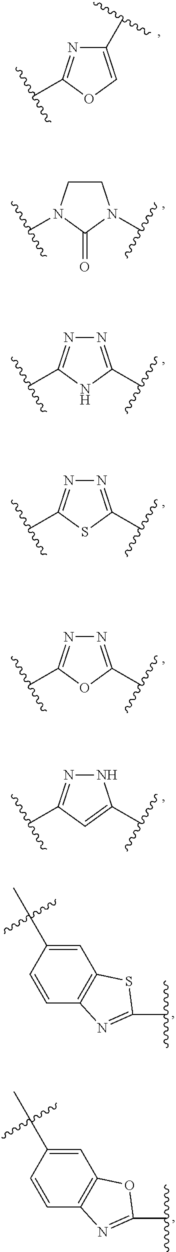

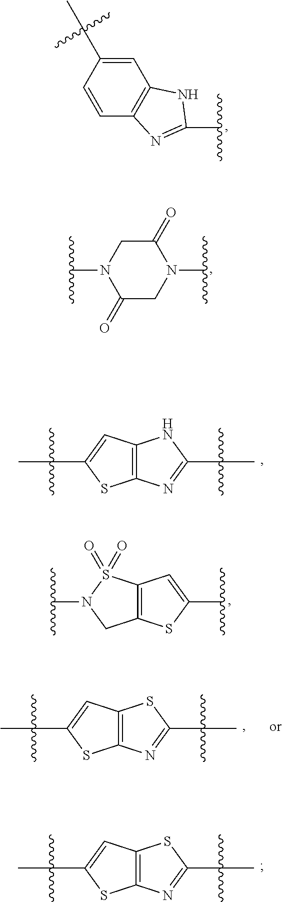 [SMILES: *c1ccc2nc(*)[nH]c2c1 ; *N1CC(=O)N(*)CC1=O ; *c1nc2sc(*)cc2[nH]1 ; *c1cc2c(s1)CN(*)S2(=O)=O ; *c1cc2sc(*)nc2s1 ; *c1cc2sc(*)nc2s1]

and B is selected from

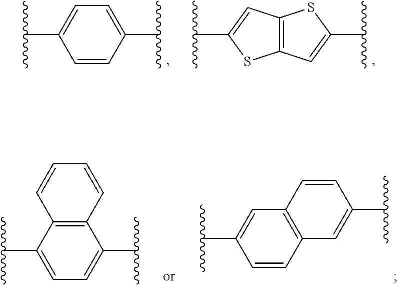

or alternatively,

B is selected from

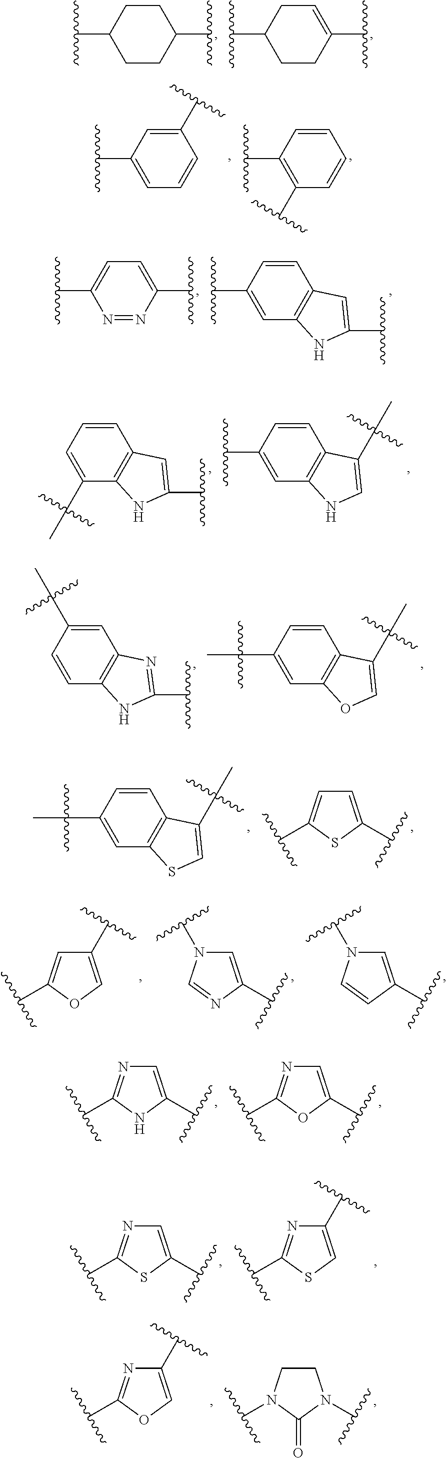

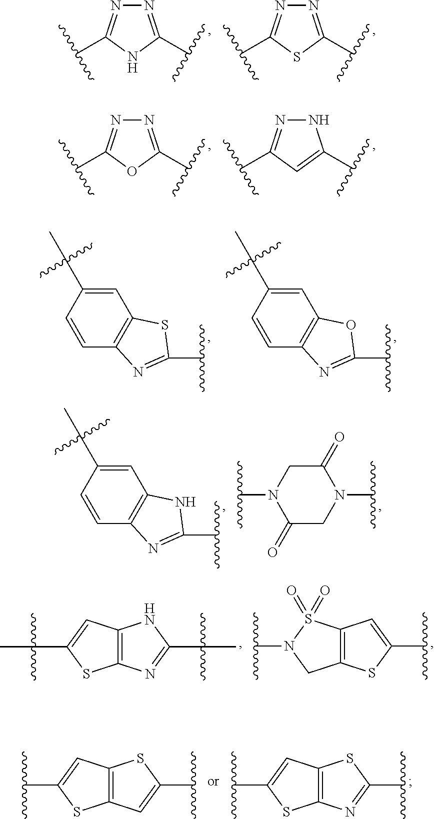

and A is selected from

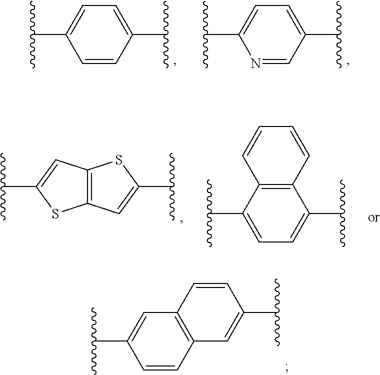

wherein rings in A and B can each be unsubstituted or substituted with one or more substituents selected from alkyl, amino, haloalkyl, halogen, hydroxy, alkoxy, haloalkoxy, cyano, sulfonyl or oxo; and R⁴ is hydrogen or alkyl optionally substituted with halogen. In some such embodiments, A is a phenyl. In some further embodiments, A is substituted with one or more halogen or sulfonyl, for example, fluoro or methanesulfonyl (—SO₂CH₃). In some such embodiments, B is a phenyl. In some other such embodiments, B is a naphthyl. In some further embodiments, B is substituted with one or more halogen or sulfonyl, for example, fluoro or methanesulfonyl.

In some embodiments, one of A or B is selected from

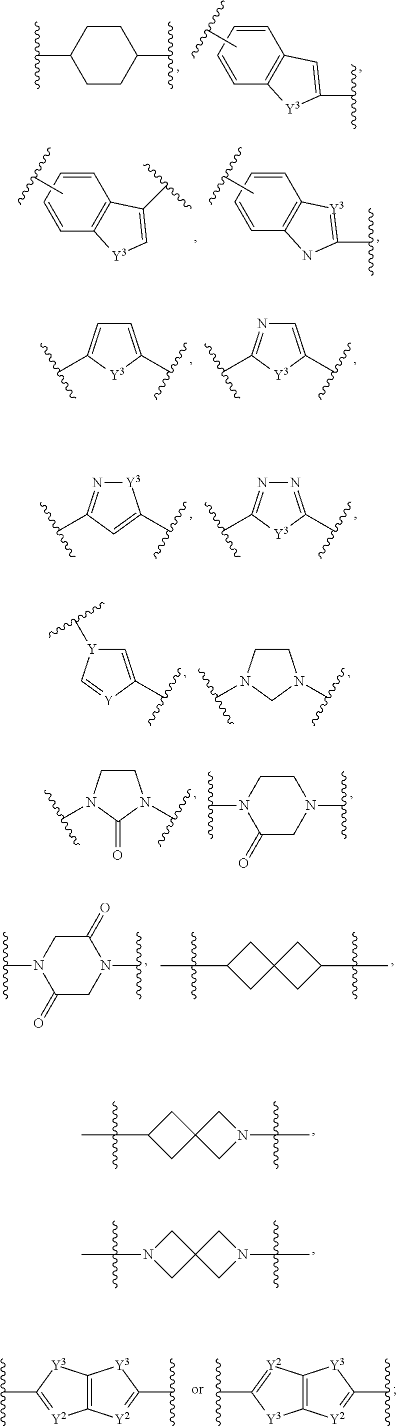

and the other A or B is a ring system selected from the group consisting of 6-11 membered aryl, 5-11 membered heteroaryl, 4-11 membered heterocyclyl, and 4-11 membered carbocyclyl; wherein rings in A or B are unsubstituted or substituted with one or more substituents selected from alkyl, halogen, hydroxy, alkoxy, haloalkoxy, cyano, or oxo; D is selected from

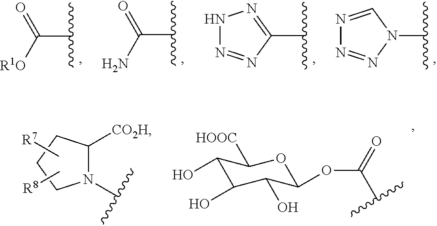

or carboxylic acid isosteres; E is absent; L⁴ is selected from

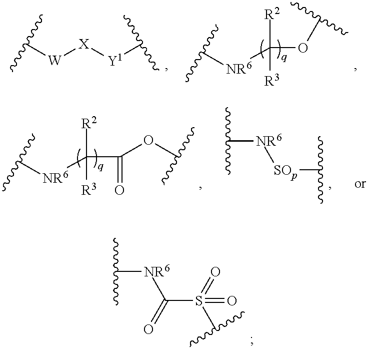

L¹ is selected from a single bond, a —O— linker, a —C(O)— linker, a —CH₂O— linker, a

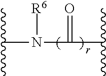

linker, a —C≡C— linker, or a —CH═CH— linker; L² is selected from a single bond, a —O— linker, a

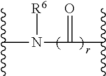

linker, a C(O)— linker, a —CH₂— linker, a —CH₂O— linker, a —C≡C— linker, or a —CH═CH— linker; L⁵ is selected from a single bond, a —CH₂O— linker, a —CH═CH— linker, a —C≡C— linker,

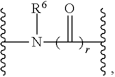

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl;

or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments, the compound of Formula (III) is also represented by Formula (IIIb):

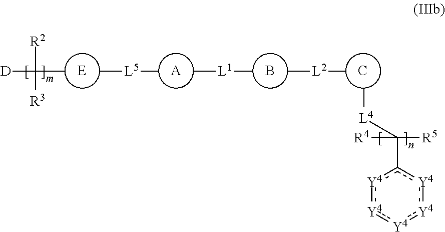

wherein E is not absent.

In some embodiments of Formula (III), (IIIa) and (IIIb), A is optionally substituted

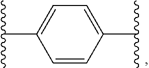 [SMILES: *c1ccc(*)cc1]

In some such embodiments, A is substituted with one or more halogen, alkyl, alkoxy, haloalkyl, haloalkoxy, or amino. In some other such embodiments, A is substituted with one or more sulfonyl. In some embodiments, A is optionally substituted

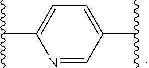 [SMILES: *c1ccc(*)nc1]

In some embodiments, A is optionally substituted

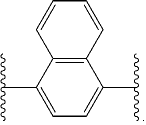 [SMILES: *c1ccc(*)c2ccccc12]

In some of these embodiments of A, B is

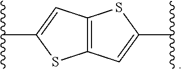 [SMILES: *c1cc2sc(*)cc2s1]

In some of these embodiments of A, B is optionally substituted

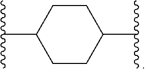 [SMILES: *C1CCC(*)CC1]

In some other these embodiments of A, B is optionally substituted

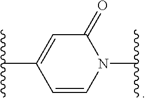 [SMILES: *c1ccn(*)c(=O)c1]

Each A and B can be optinally substituted with one or more substituents selected from alkyl, amino, haloalkyl, halogen, alkoxy, haloalkoxy, cyano, sulfonyl or oxo.

In some embodiments of Formula (III), (IIIa) and (IIIb), B is optionally substituted

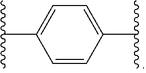 [SMILES: *c1ccc(*)cc1]

In some embodiments, B is optionally substituted

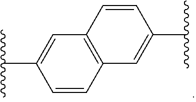 [SMILES: *c1ccc2cc(*)ccc2c1]

In some such embodiments, B is optionally substituted with one or more halogen, alkyl, alkoxy, haloalkyl, haloalkoxy or amino. In some embodiments, B is optionally substituted

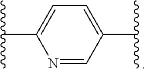 [SMILES: *c1ccc(*)nc1]

In some embodiments, B is optionally substituted

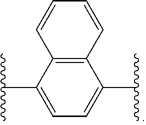 [SMILES: *c1ccc(*)c2ccccc12]

In some embodiments, B is

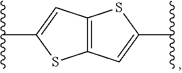 [SMILES: *c1cc2sc(*)cc2s1]

optionally substituted with one or more substituents selected from alkyl, haloalkyl, halogen, hydroxy, alkoxy, haloalkoxy, cyano, or oxo. In some of these embodiments of B, A is selected from

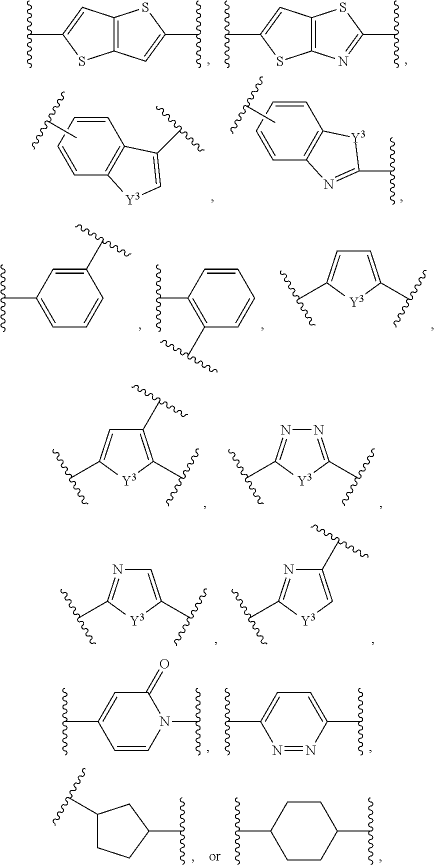

each optionally substituted. In one embodiment, A is

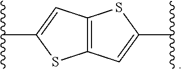 [SMILES: *c1cc2sc(*)cc2s1]

In another embodiment, A is

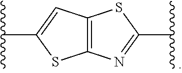 [SMILES: *c1cc2sc(*)nc2s1]

In other embodiments, A is selected from

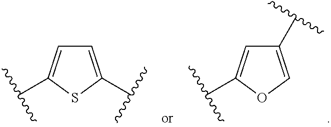 [SMILES: *c1ccc(*)s1 ; *c1coc(*)c1]

In still other embodiments, A is selected from

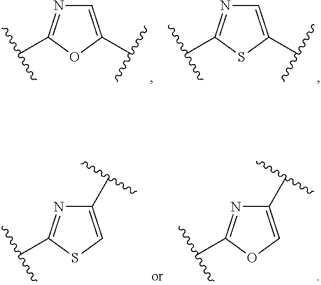 [SMILES: *c1cnc(*)o1 ; *c1cnc(*)s1 ; *c1csc(*)n1 ; *c1coc(*)n1]

In still other embodiments, A is selected from

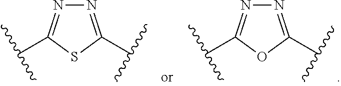 [SMILES: *c1nnc(*)s1 ; *c1nnc(*)o1]

In another embodiment, A is

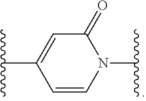 [SMILES: *c1ccn(*)c(=O)c1]

In still other embodiments, A is selected from

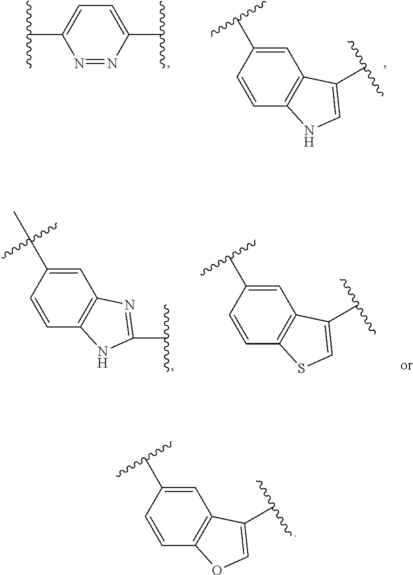 [SMILES: *c1ccc(*)nn1 ; *c1ccc2[nH]cc(*)c2c1 ; *c1ccc2[nH]c(*)nc2c1 ; *c1ccc2scc(*)c2c1 ; *c1ccc2occ(*)c2c1]

In still other embodiments, A is selected from

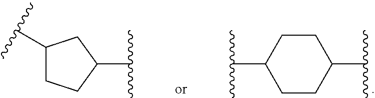 [SMILES: *C1CCC(*)C1 ; *C1CCC(*)CC1]

Each A and B can be optinally substituted with one or more substituents selected from alkyl, amino, haloalkyl, halogen, alkoxy, haloalkoxy, cyano, or oxo.

In some embodiments described herein of the compounds of Formula (III), (IIIa) or (IIIb), rings in A are unsubstituted.

In some embodiments described herein of the compounds of Formula (III), (IIIa) or (IIIb), rings in B are unsubstituted.

In some embodiments described herein of the compounds of Formula (III), (IIIa) or (IIIb), rings in A can be optionally substituted. In some such embodiments, A can be substituted with one or more substituents selected from alkyl, amino, haloalkyl, halogen, hydroxy, alkoxy, haloalkoxy, cyano, or oxo. In some other such embodiments, A can be substituted with one or more sulfonyl, for example, methanesulfonyl.

In some embodiments described herein of the compounds of Formula (III), (IIIa) or (IIIb), rings in B can be optionally substituted. In some such embodiments, B can be substituted with one or more substituents selected from alkyl, amino, haloalkyl, halogen, hydroxy, alkoxy, haloalkoxy, cyano, or oxo. In some other such embodiments, B can be substituted with one or more sulfonyl, for example, methanesulfonyl.

In some embodiments described herein of the compounds of Formula (III), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments described herein of the compounds of Formula (III) or (IIIb), E can be unsubstituted or substituted. In some embodiments, E can be substituted with one or more substituents selected from alkyl, amino, haloalkyl, halogen, hydroxy, alkoxy, haloalkoxy, cyano, or oxo. In some other such embodiments, E can be substituted with one or more sulfonyl, for example, methanesulfonyl.

In some embodiments, E is an optionally substituted 5-10 membered heteroarylene. In some such embodiments, E is selected from

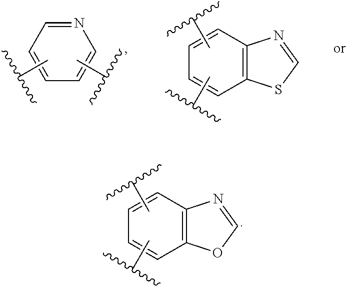

In some such embodiments, E is selected from

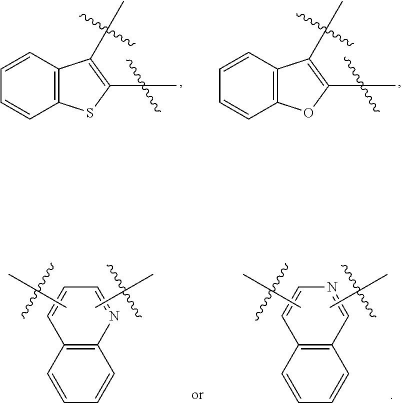

In some embodiments, E is an optionally substituted 6-10 membered arylene. In some such embodiments, E is optionally substituted phenylene.

In some embodiments, D is —C(O)OR¹. In some such embodiments, R¹ is hydrogen. In some such embodiments, R¹ is optionally substituted alkyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

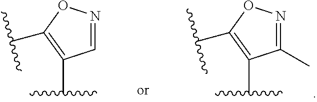

In some embodiments, C is selected from

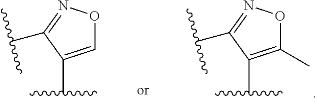

In some embodiments, C is selected fron

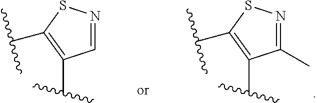

In some embodiments, C is selected from

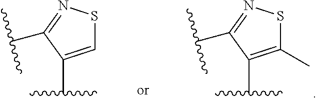

In some embodiments, C is selected from

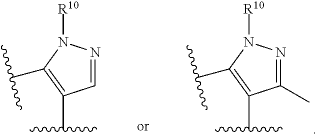

In some such embodiments, R¹⁰ is hydrogen. In some such embodiments, R¹⁰ is C₁₋₃ alkyl or C₃₋₆ cycloalkyl

In some embodiments, C is

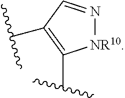

In some embodiments, C is

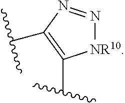

In some embodiments, C is

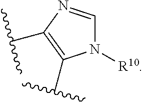

In some embodiments, C is

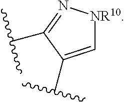

In some such embodiments, R¹⁰ is C₁₋₃ alkyl or C₃₋₆ cycloalkyl. In some such embodiments, R¹⁰ is hydrogen. In some such embodiments, C is selected from

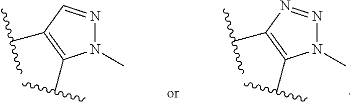

In some embodiments, C is selected from

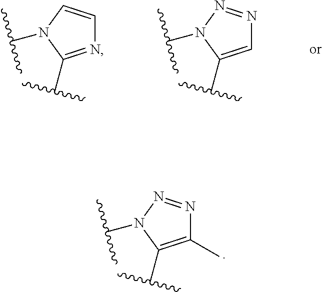

In some embodiments, C is selected from

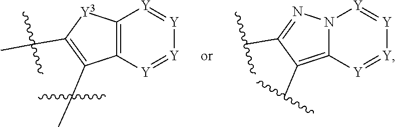

wherein Y³ is selected from O or S. In some such embodiments, Y is a CR⁶. In some other such embodiments, at least one Y is nitrogen. In some embodiments, C is optionally substituted

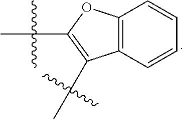 [SMILES: *c1oc2ccccc2c1*]

In some embodiments, C is selected from

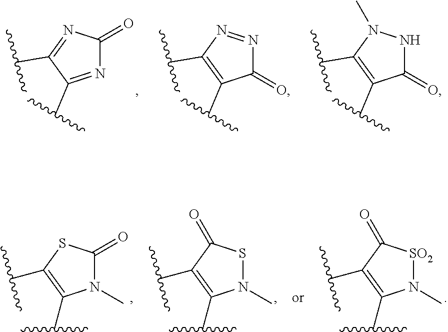

In some embodiments, m is 0. In some other embodiments, m is 1.

In some embodiments, each of R² and R³ is hydrogen. In some embodiments, at least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L⁵ is a —SO₂-linker. In some embodiments, L⁵ is a —NH— linker. In some embodiments, L⁵ is a —O— linker.

In some embodiments, L² is a single bond.

In some embodiments, L¹ is a single bond. In some embodiments, L¹ is a —O-linker. In some other embodiments, L¹ is —C≡C— linker. In still some other embodiments, L¹ is a

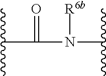

linker, wherein R^{6b} is hydrogen or optionally substituted C₁₋₃ alkyl. In some of such embodiments, L¹ is a —C(O)—NH— linker.

In some embodiments, R⁶ is hydrogen.

R⁴ is selected from hydrogen or alkyl optionally substituted with halogen. In some other embodiments, R⁴ is unsubstituted alkyl.

In some embodiments of the compounds of Formula (III), (IIIa) or (IIIb),

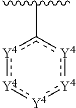

can be

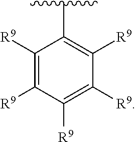

In some such embodiments, each of R⁹ is hydrogen. In some other such embodiments, at least one R⁹ is selected from C₁₋₃ alkyl or halogen.

In some embodiments,

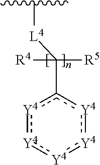

can be

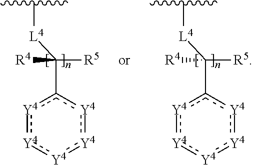

In some embodiments of the compound of Formula (III) are selected from compounds of Table 3, Table 3A, Table 3B and Table 3C as shown below, and pharmaceutically acceptable salt thereof.

In some embodiments of the compounds of Formula (III) are selected from the group consisting of IT007-IT010, IT025, IT046, IT050, IT051, IT053, IT054, IT056, IT059, IT060, IT066, IT067, IT071, IT091, IT111, IT119-IT122, IT132-IT135, IT140-IT144, IT147-IT149, IT152, IT156-IT171, IT175-IT193, IT200-IT224, IT236, IT237, IT239-IT255, IT259-IT276, IT278, IT279, IT281-IT299, IT317-IT343, IT346-IT354, IT357-IT367, IT369, IT370, IT372, IT373, IT376-IT387, IT389-IT397, IT410-IT416, IT421, IT422, IT426, IT427, IT433, IT441, IT442, IT445, IT458, IT470, IT475, IT480 or IT488 as shown in Table 13.

Formula (IV)

In some embodiments disclosed herein include a compound of Formula (IV) as described above or a pharmaceutically acceptable salt thereof.

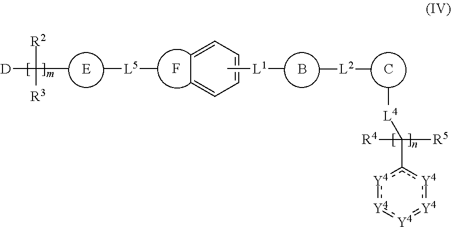

In some embodiments, B is selected from the group consisting of

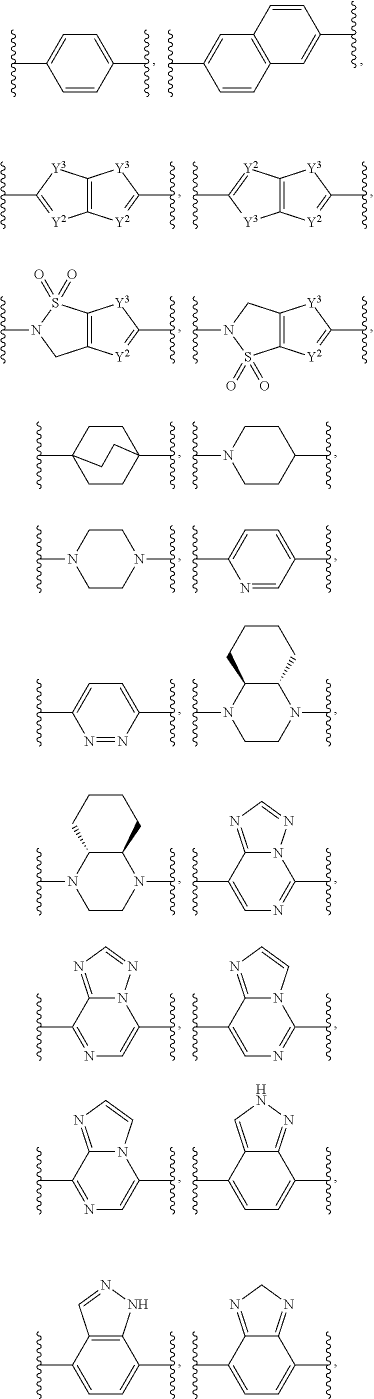

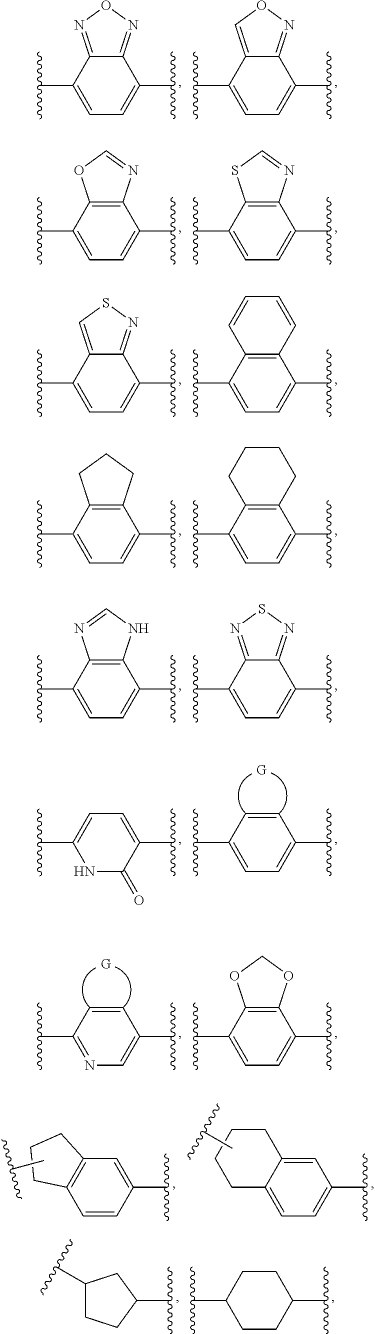

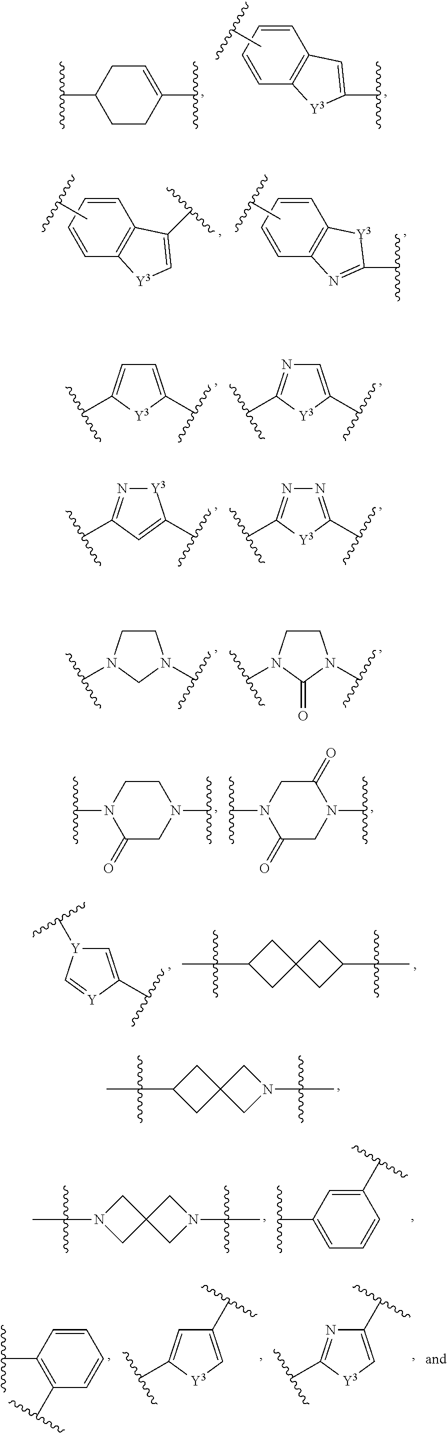

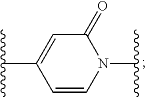 [SMILES: *c1ccn(*)c(=O)c1 ; ";"]

wherein the rings in B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

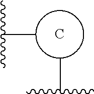

is selected from

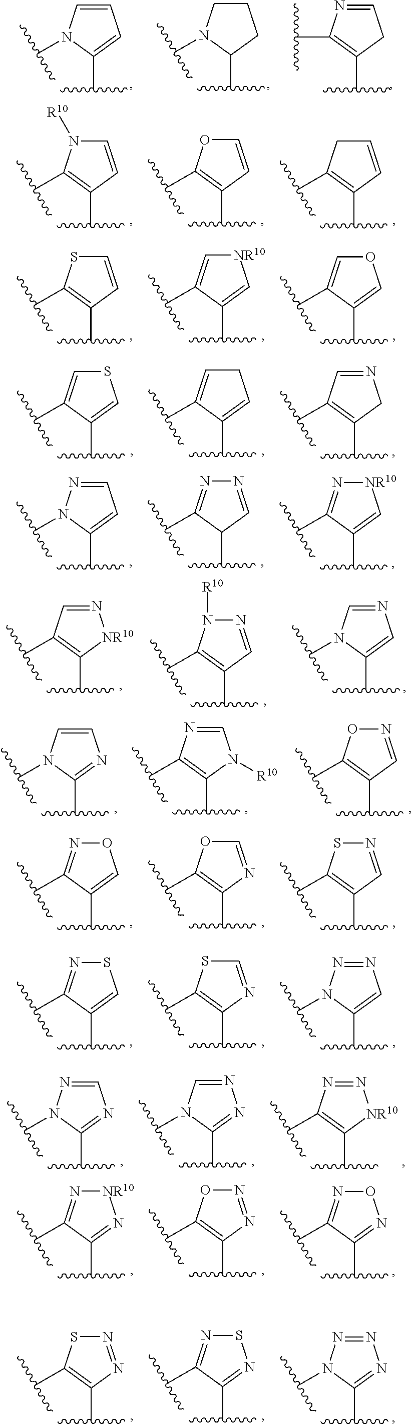

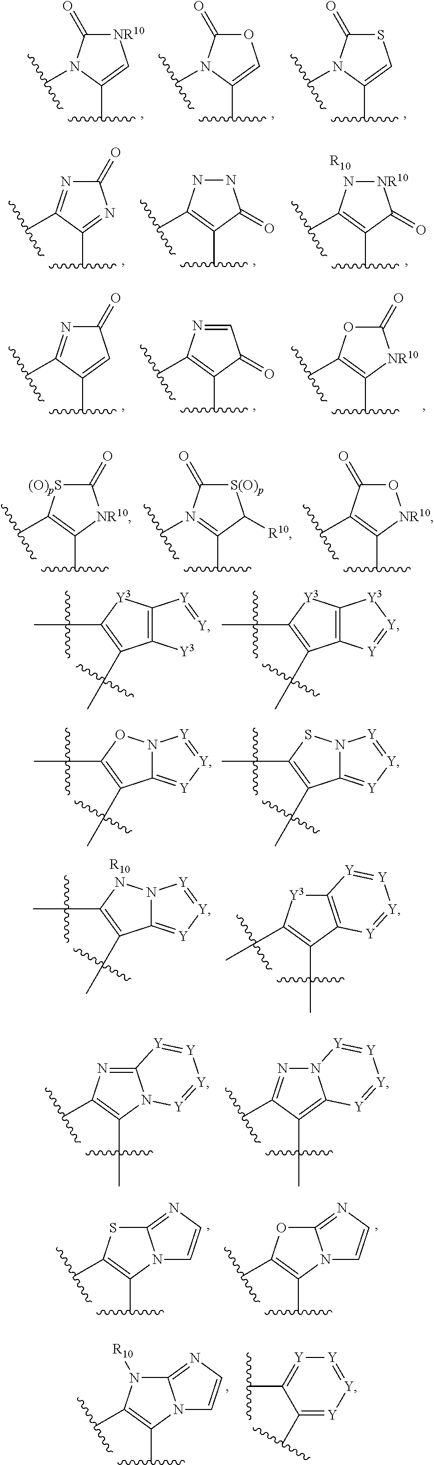

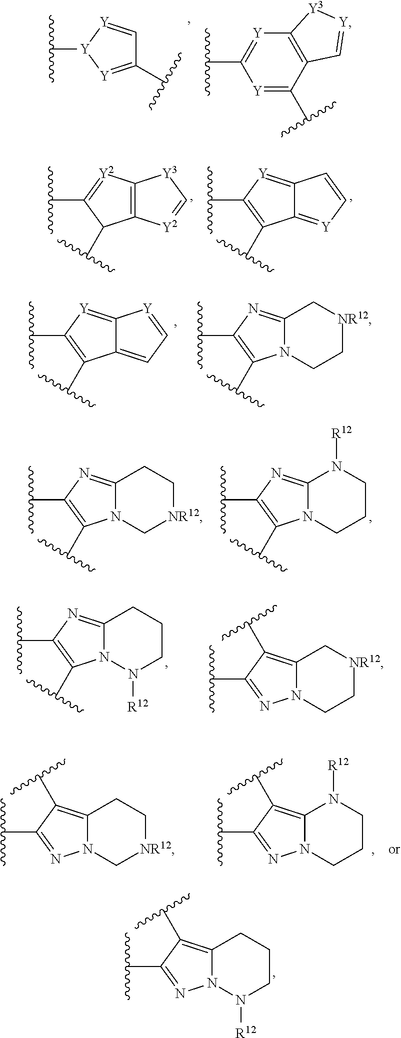

or optionally substituted variants thereof; each Y² is independently selected from —CH═ or N; each Y³ is independently selected from C(R⁶)₂, NR⁶, O, or S; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (IV) is also represented by Formula (IVa):

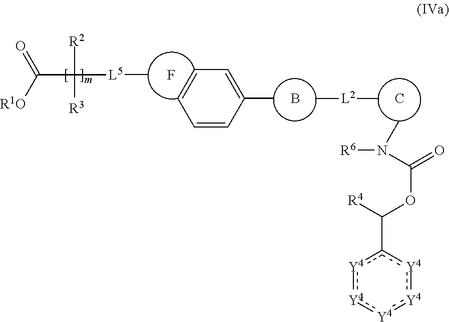

wherein

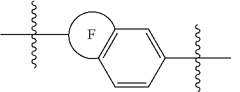

is selected from

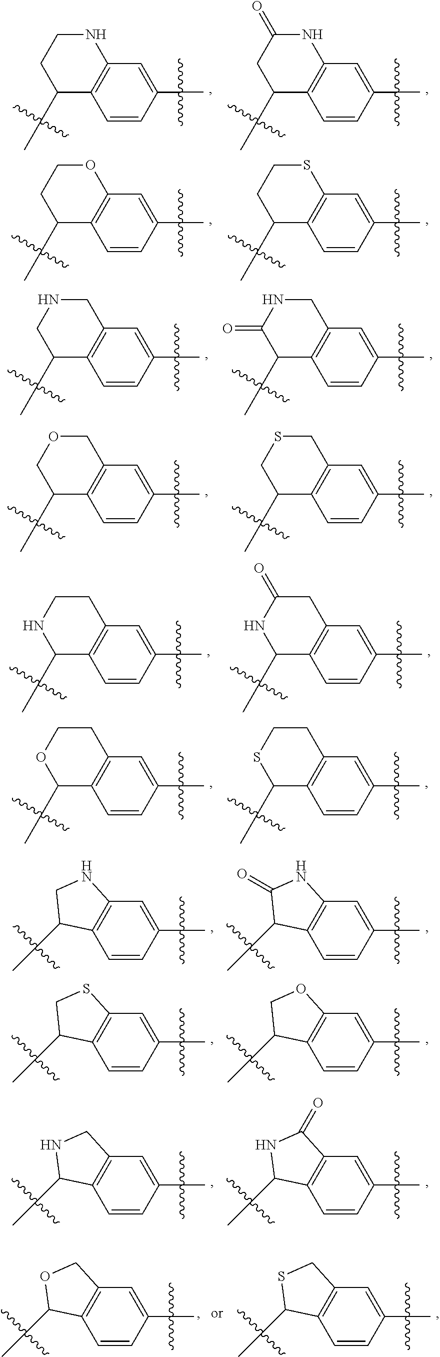

wherein

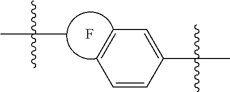

is unsubstituted or substituted with one or more substituents selected from alkyl, amino, halogen, haloalkyl, hydroxy, alkoxy, haloalkoxy, cyano, or oxo.

In some embodiments, each B and

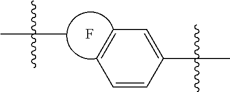

is unsubstituted or substituted with one or more substituents selected from alkyl, halogen, haloalkyl, hydroxy, alkoxy, haloalkoxy, cyano, or oxo; D is selected from

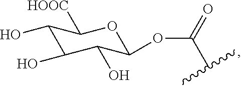 [SMILES: *C(=O)OC1OC(C(=O)O)C(O)C(O)C1O]

or carboxylic acid isosteres; E is absent; L⁴ is selected from

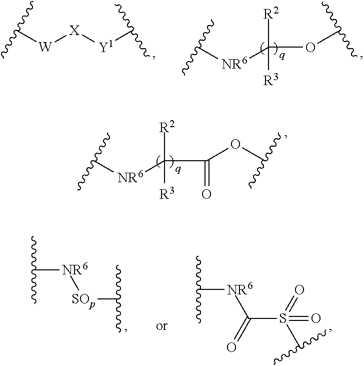

each L¹ and L² is independently selected from a single bond, a —O— linker, a

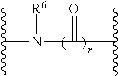

linker, a —C(O)— linker, a —CH₂— linker, a —CH₂O— linker, a —C≡C— linker, or a —CH═CH— linker; L⁵ is selected from a single bond, a —CH₂O-linker, a —OCH₂— linker, a —CH═CH— linker, a —C≡C— linker,

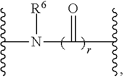

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl;

each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments, B is selected from phenyl or naphthyl, and wherein B is unsubstituted or substituted with one or more substituents selected from alkyl, amino, halogen, haloalkyl, hydroxy, alkoxy, haloalkoxy, cyano, or oxo. In some of such embodiments, B is unsubstituted phenyl. In some other such embodiments, B is a phenyl substituted with one or more halogen.

In some embodiments,

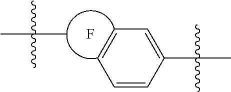

is selected from

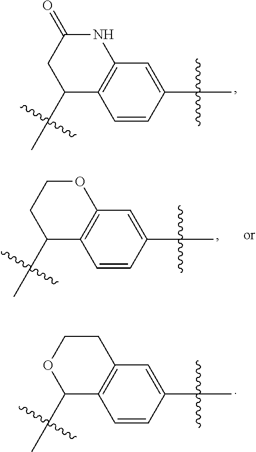 [SMILES: *c1ccc2c(c1)NC(=O)CC2* ; *c1ccc2c(c1)OCCC2* ; *c1ccc2c(c1)CCOC2*]

In some embodiments

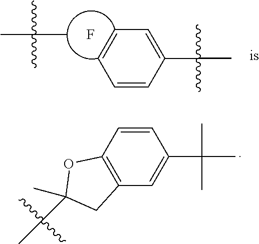

In some embodiments described herein of the compound of Formula (IV) or (IVa), rings in B and

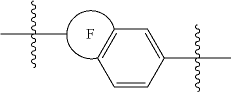

are unsubstituted.

In some embodiments described herein of the compound of Formula (IV) or (IVa), rings in B and

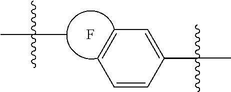

can be substituted. In some such embodiments, rings in B and

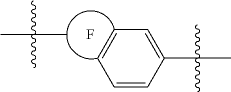

can be substituted with one or more substituents selected from alkyl, amino, haloalkyl, halogen, hydroxy, alkoxy, haloalkoxy, cyano, or oxo. In some other such embodiments rings in B and

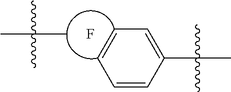

can be substituted with one or more sulfonyl, for example, methanesulfonyl.

Some embodiments described herein of the compounds of Formula (IV), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments described herein of the compounds of Formula (IV), E is unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

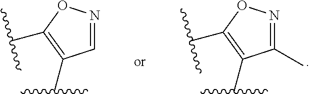

In some embodiments, C is selected from

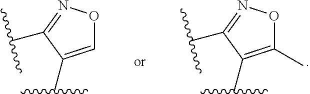

In some embodiments, C is selected from

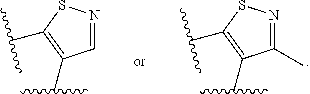

In some embodiments, C is selected from

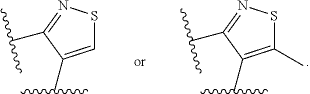

In some embodiments, C is selected from

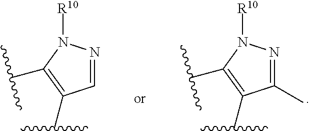

In some such embodiments, R¹⁰ is hydrogen. In some such embodiments, R¹⁰ is C₁₋₃ alkyl or C₃₋₆ cycloalkyl.

In some embodiments, C is

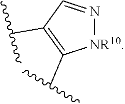

In some other embodiments C is

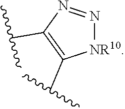

In some other embodiments, C is selected from

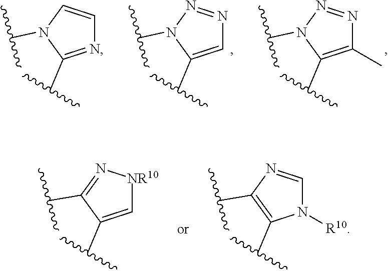

In some such embodiments, R¹⁰ is C₁₋₃ alkyl or C₃₋₆ cycloalkyl. In some such embodiments, R¹⁰ is hydrogen.

In some embodiments, C is selected from

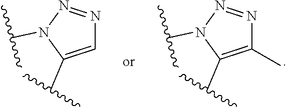

In some embodiments, C is selected from

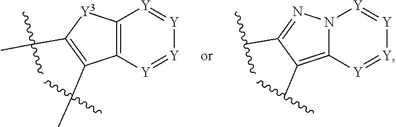

wherein Y³ is selected from O or S. In some such embodiments, Y is a CR⁶. In some other such embodiments, at least one Y is nitrogen. In some embodiments, C is optionally substituted

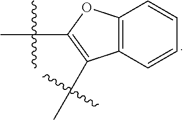 [SMILES: *c1oc2ccccc2c1*]

In some embodiments, C is selected from

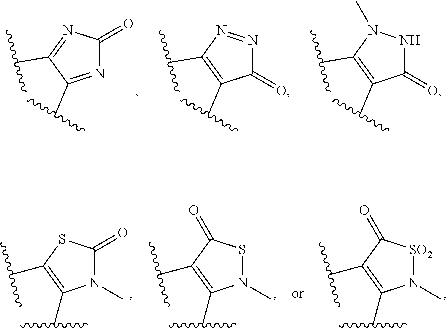

In some embodiments, m is 0. In some other embodiments, m is 1.

In some embodiments, each of R² and R³ is hydrogen. In some embodiments, at least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is hydrogen. In some embodiments, R¹ is hydrogen.

R⁴ is selected from hydrogen or alkyl optionally substituted with halogen. In some other embodiments, R⁴ is unsubstituted alkyl.

In some embodiments of the compound of Formula (IV) or (IVa),

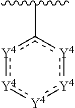

can be

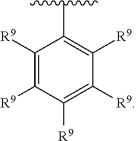

In some such embodiments, each of R⁹ is hydrogen. In some other such embodiments, at least one R⁹ is selected from C₁₋₃ alkyl or halogen.

In some embodiments,

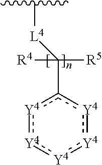

can be

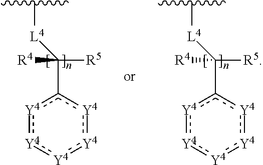

Some embodiments of the compounds of Formula (IV) are selected from compounds of Table 4 as shown below, and pharmaceutically acceptable salt thereof.

Some embodiments of the compounds of Formula (IV) are selected from compounds IT011, IT012, IT037 or IT498, as shown in Table 13.

Formula (V)

Some embodiments disclosed herein include a compound of Formula (V) as described above or a pharmaceutically acceptable salt thereof.

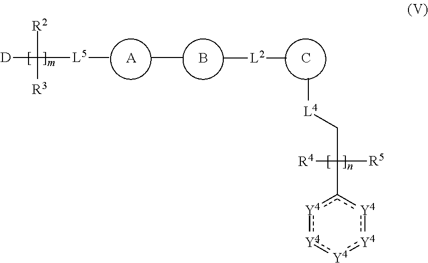

In some embodiments,

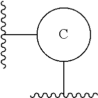

is selected from

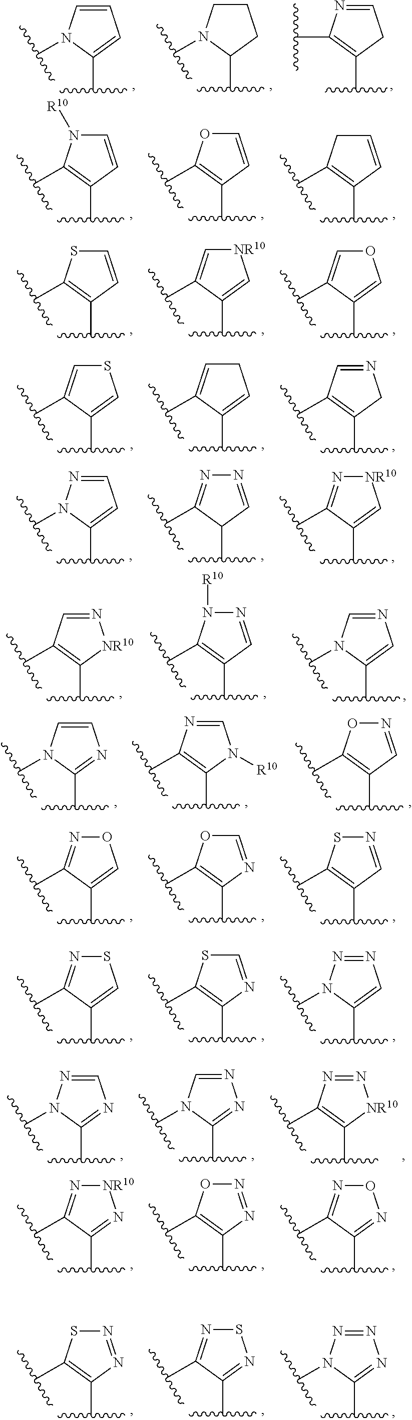

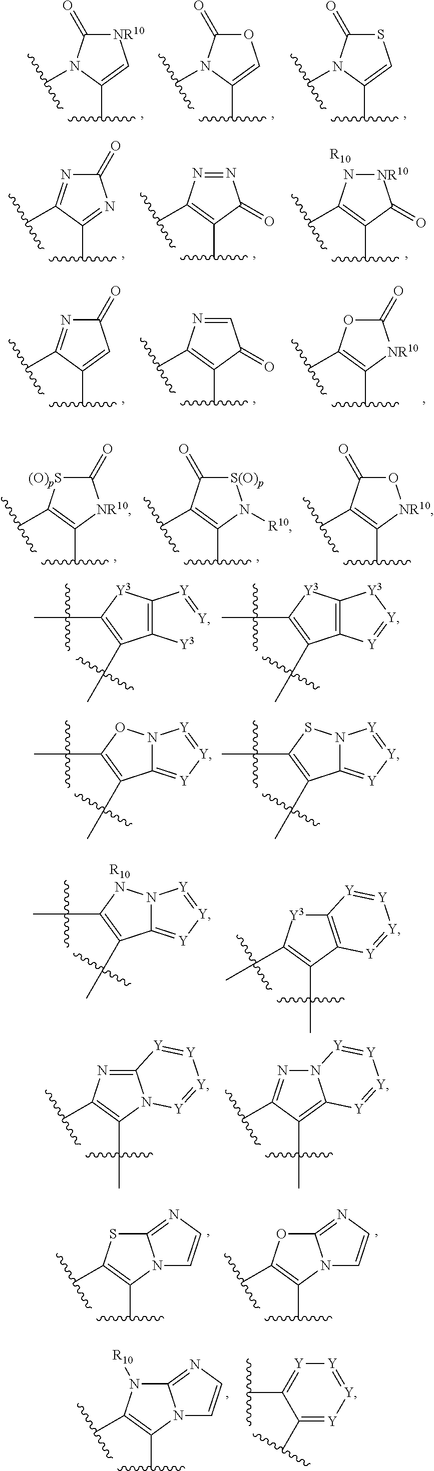

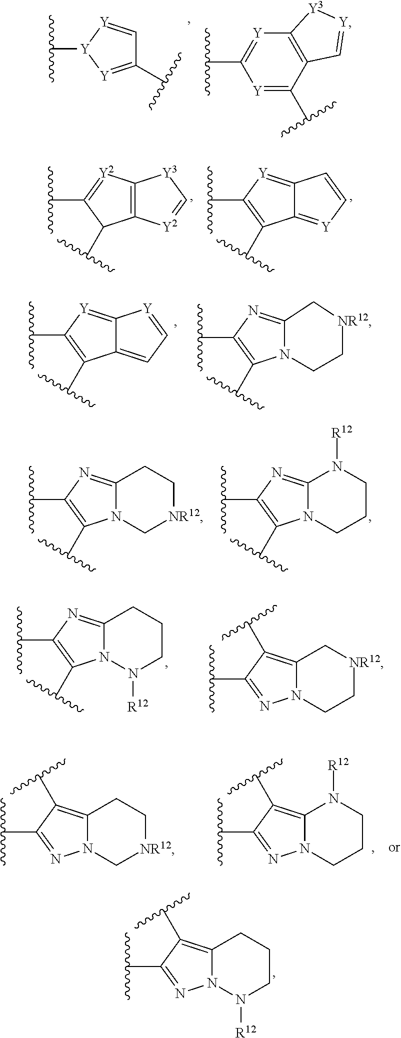

or optionally substituted variants thereof; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (V) is also represented by Formula (Va):

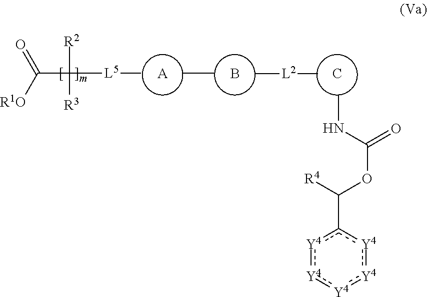

wherein one of A or B is an acetylene and the other one of A or B is selected from

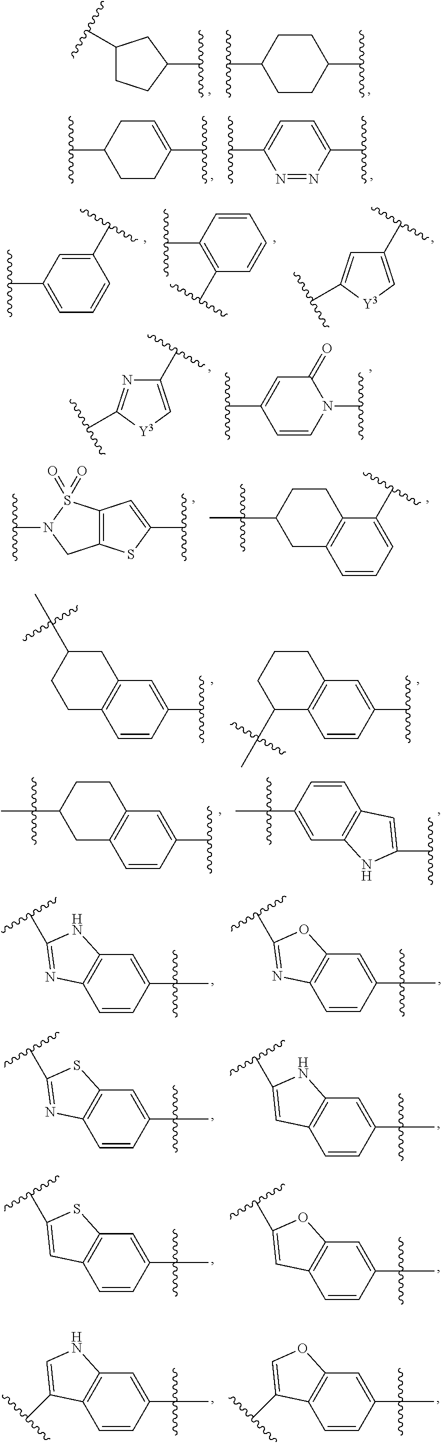

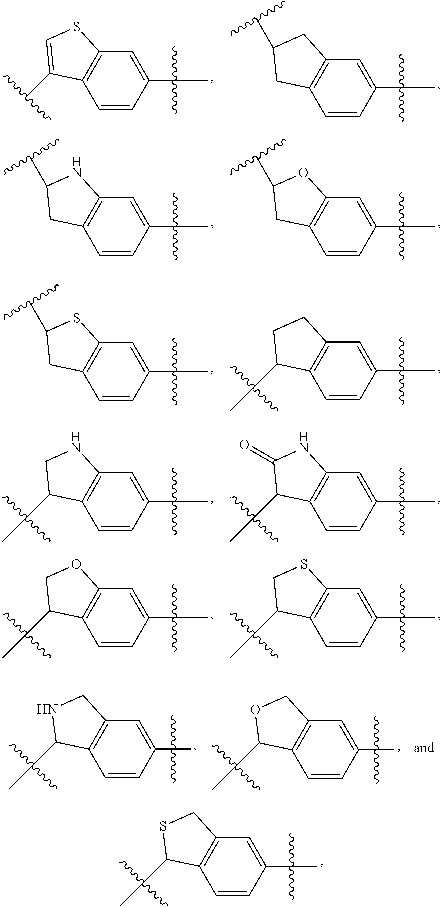

wherein rings in A or B is unsubstituted or substituted with one or more substituents selected from alkyl, amino, halogen, haloalkyl, hydroxy, alkoxy, haloalkoxy, cyano, or oxo. In some such embodiments, one of A or B is an acetylene and the other one of A or B is selected from

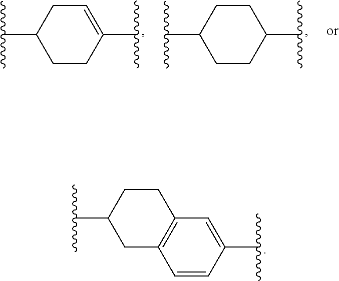

In some such embodiments, A is an acetylene. In some other such embodiments, B is an acetylene.

In some embodiments, one of A or B is an acetylene and the other A or B is a ring system selected from

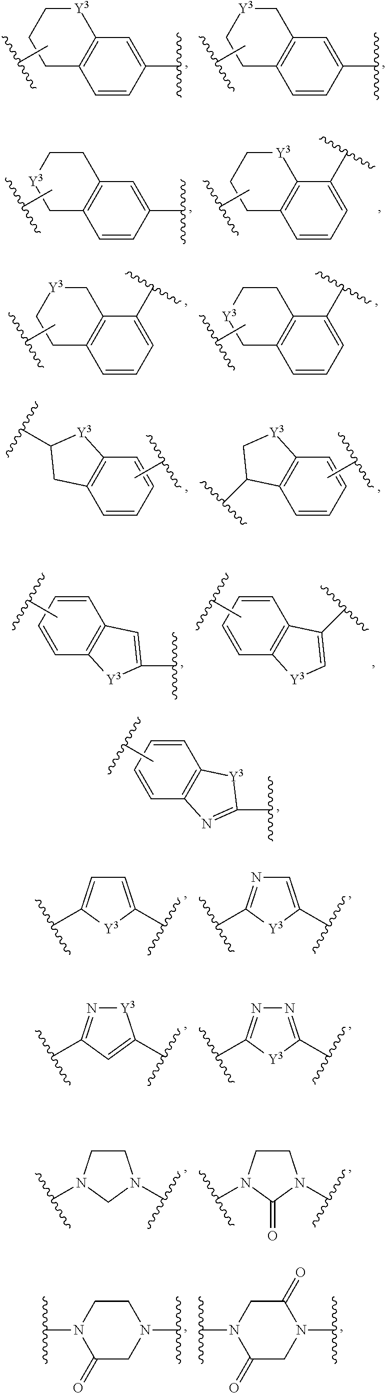

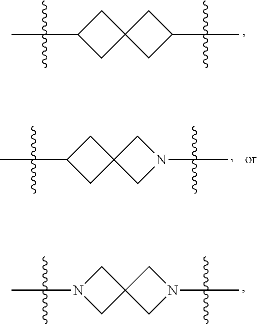

wherein A or B is unsubstituted or substituted with one or more substituents selected from alkyl, halogen, haloalkyl, hydroxy, alkoxy, haloalkoxy, cyano, or oxo; D is selected from

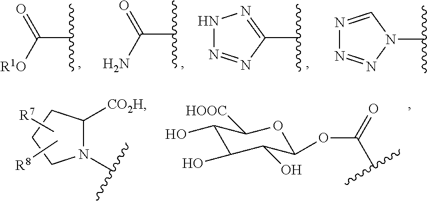

or carboxylic acid isosteres; E is absent; L⁴ is selected from

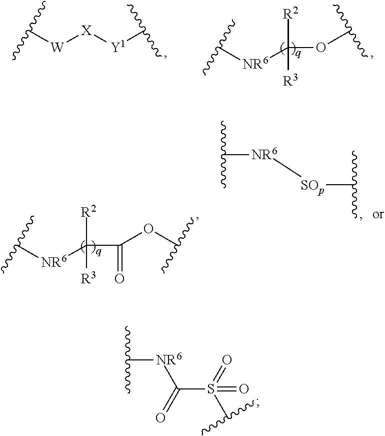

L² is selected from a single bond, a —O— linker, a

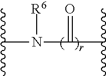

linker, a —C(O)— linker, a CH₂— linker, a —CH₂O— linker, or a —CH═CH— linker; L⁵ is selected from a single bond, a —CH₂O-linker, a —CH═CH— linker,

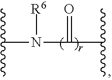

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments described herein of the compound of Formula (V) or (Va), rings in A or B are unsubstituted.

In some embodiments described herein of the compound of Formula (V) or (Va), rings in A or B can be substituted. In some such embodiments, rings in A or B can be substituted with one or more substituents selected from alkyl, amino, haloalkyl, halogen, hydroxy, alkoxy, haloalkoxy, cyano, or oxo. In some embodiments, rings A or B can be substituted with one or more sulfonyl, for example, methanesulfonyl.

In some embodiments described herein of the compounds of Formula (V), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments described herein of the compounds of Formula (V), E can be unsubstituted or substituted. In some embodiments, E can be substituted with with one or more substituents selected from alkyl, amino, haloalkyl, halogen, hydroxy, alkoxy, haloalkoxy, cyano, or oxo. In some embodiments, E can be substituted with one or more sulfonyl, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

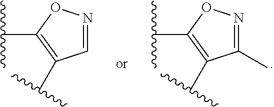

In some embodiments, C is selected from

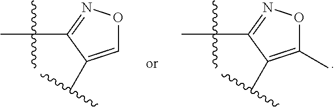

In some embodiments, C is selected frons

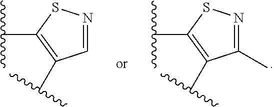

In some embodiments, C is selected from

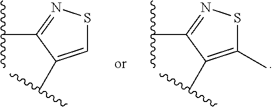

In some embodiments, C is selected from

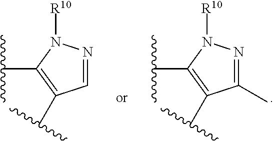

In some such embodiments, R¹⁰ is hydrogen. In some such embodiments, R¹⁰ is C₁₋₃ alkyl or C₃₋₆ cycloalkyl.

In some embodiments, C is

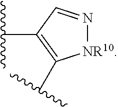

In some embodiments, C is

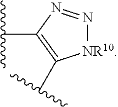

In some such embodiments, R¹⁰ is C₁₋₃ alkyl or C₃₋₆ cycloalkyl. In some such embodiments, R¹⁰ is hydrogen.

In some other emobdiments, C is selected from

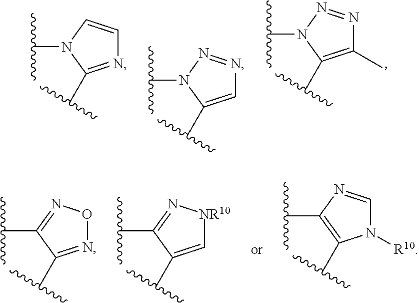

In some such embodiments, R¹⁰ is C₁₋₃ alkyl or C₃₋₆ cycloalkyl. In some such embodiments, R¹⁰ is hydrogen.

In some embodiments, C is selected from

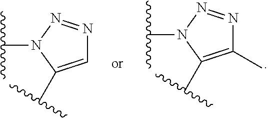

In some embodiments, C is selected from

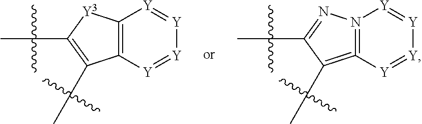

wherein Y³ is selected from O or S. In some such embodiments, Y is a CR⁶. In some other such embodiments, at least one Y is nitrogen. In some embodiments, C is optionally substituted

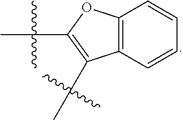 [SMILES: *c1oc2ccccc2c1*]

In some embodiments, C is selected from

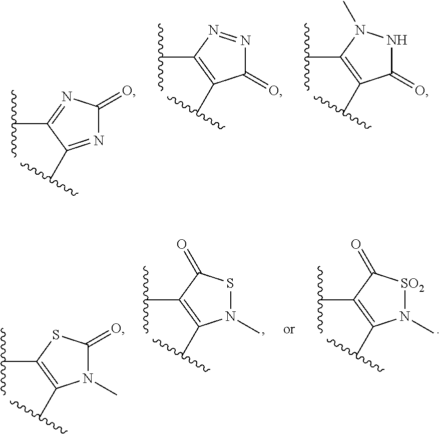

In some embodiments, m is 0. In some other embodiments, m is 1.

In some embodiments, each of R² and R³ is hydrogen. In some embodiments, at least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is 1 is hydrogen. In some embodiments, R is hydrogen.

In some embodiments, R⁴ is selected from hydrogen or alkyl optionally substituted with halogen. In some other embodiments, R⁴ is unsubstituted alkyl.

In some embodiments of the compound of Formula (V) or (Va),

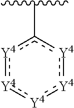

can be

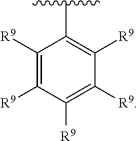

In some such embodiments, each of R⁹ is hydrogen. In some other such embodiments, at least one R⁹ is selected from C₁₋₃ alkyl or halogen.

In some embodiments,

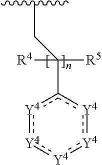

can be

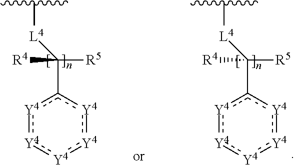

Some embodiments of the compounds of Formula (V) are selected from compounds of Table 5 as shown below, and pharmaceutically acceptable salt thereof.

Some embodiments of the compounds of Formula (V) are selected from compounds IT062, IT063 or IT092, as shown in Table 13.

Formula (VI)

Some embodiments disclosed herein include a compound of Formula (VI) as described above or a pharmaceutically acceptable salt thereof.

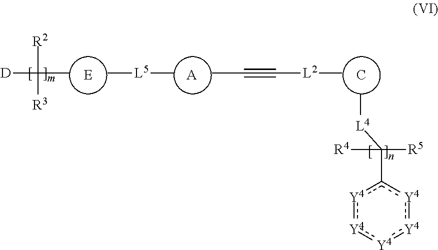

In some embodiments, A is selected from the group consisting of

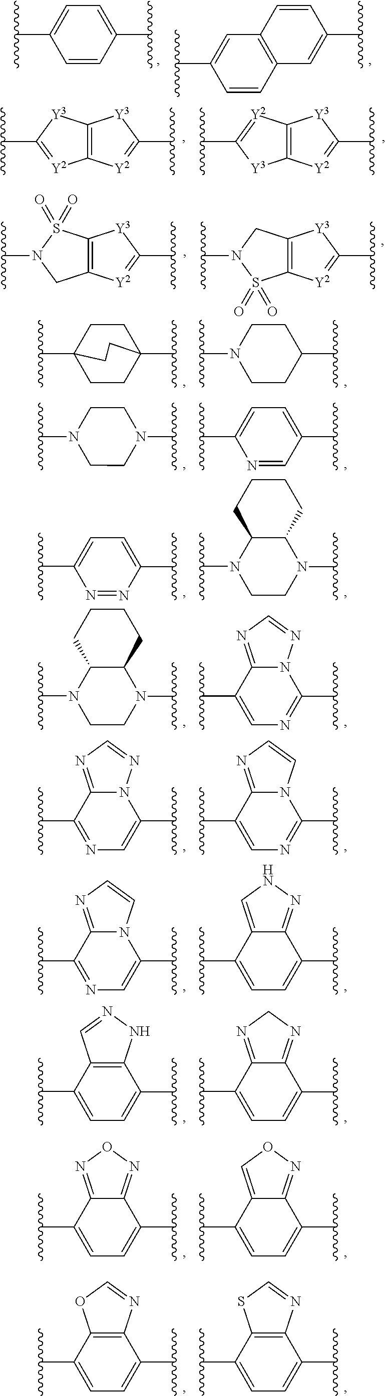

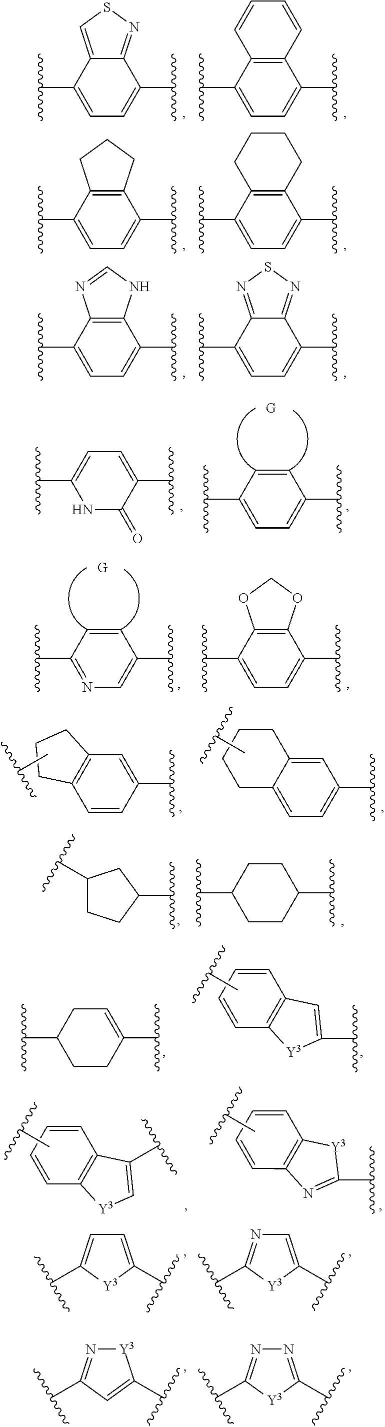

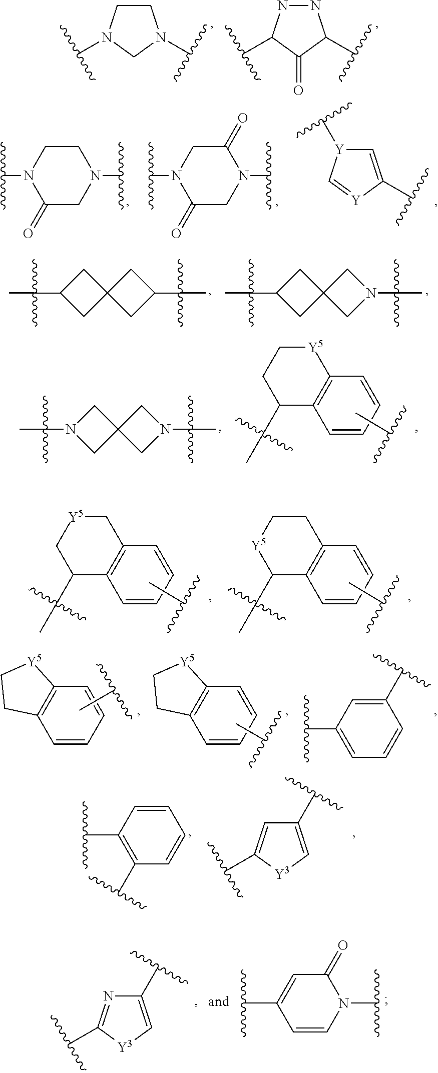

wherein the rings in A are unsubstituted or substituted with one or more substituents selected from alkyl, amino, haloalkyl, halogen, hydroxy, alkoxy, haloalkoxy, cyano, sulfonyl or oxo;

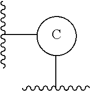

is selected from

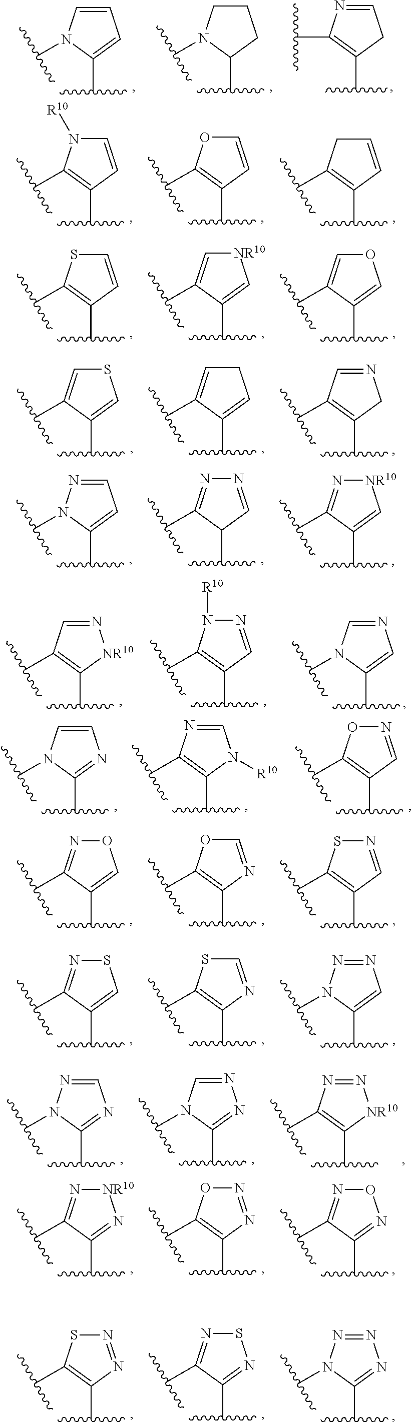

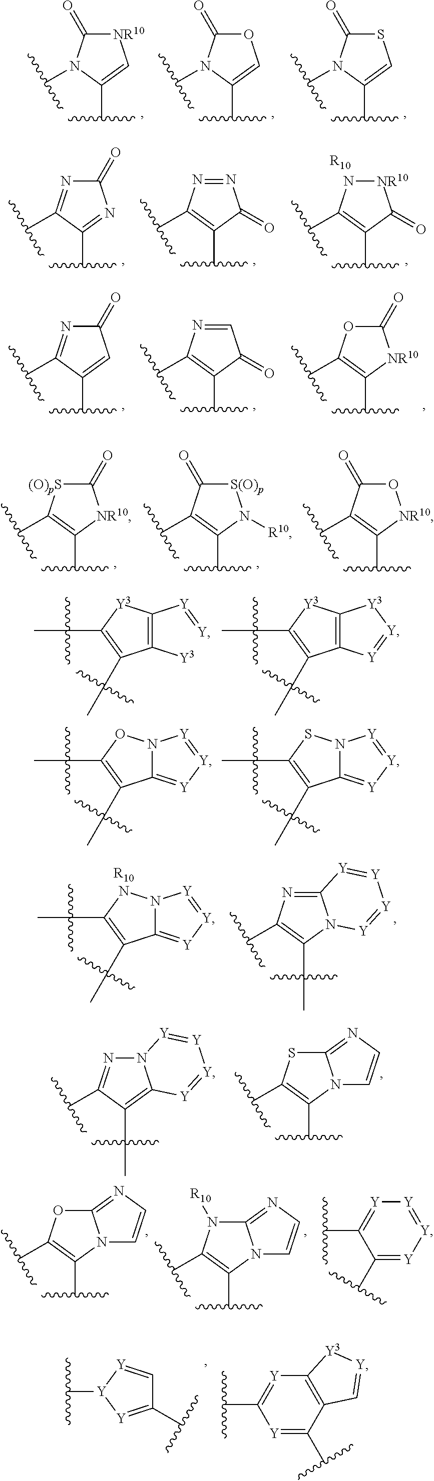

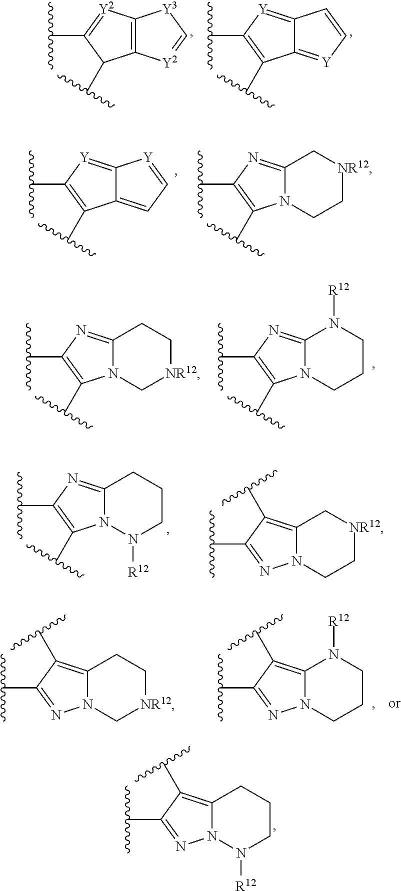

or optionally substituted variants thereof;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

each Y² is independently selected from —CH═ or N; each Y³ is independently selected from C(R⁶)₂, NR⁶, O or S; each Y⁵ is independently selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (VI) is also represented by Formula (VIa):

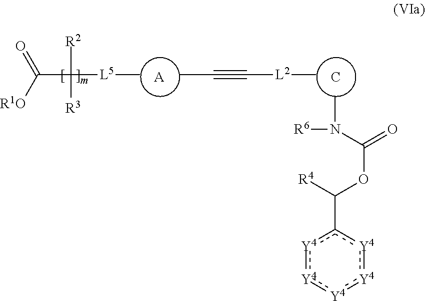

wherein A is selected from

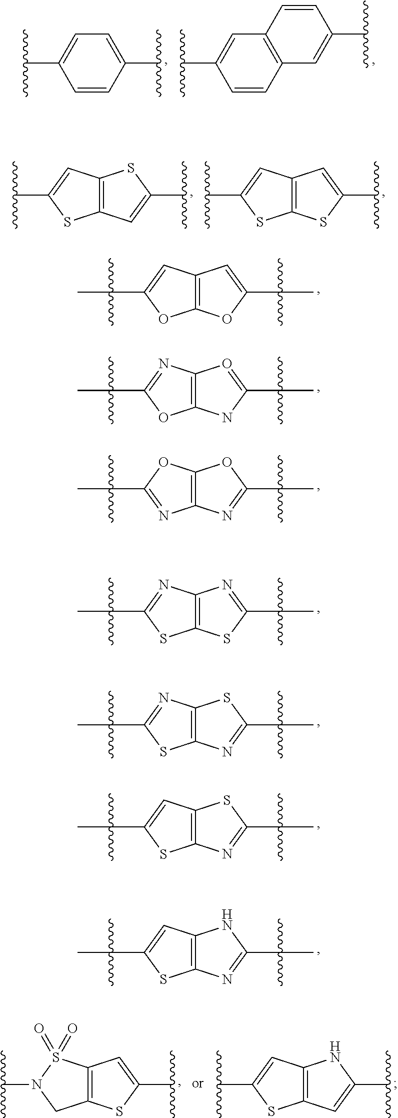

and wherein the rings in A are unsubstituted or substituted with one or more substituents selected from alkyl, amino, haloalkyl, halogen, hydroxy, alkoxy, haloalkoxy, cyano, or oxo. In some further embodiments, A is phenyl. In some further embodiments, A is naphthyl.

In some embodiments, each of A and G is unsubstituted or substituted with one or more substituents selected from alkyl, haloalkyl, halogen, hydroxy, alkoxy, haloalkoxy, cyano, or oxo; D is selected from

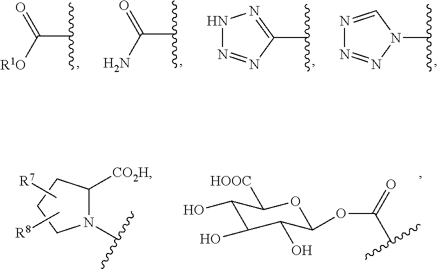

or carboxylic acid isosteres; E is absent; L⁴ is selected from

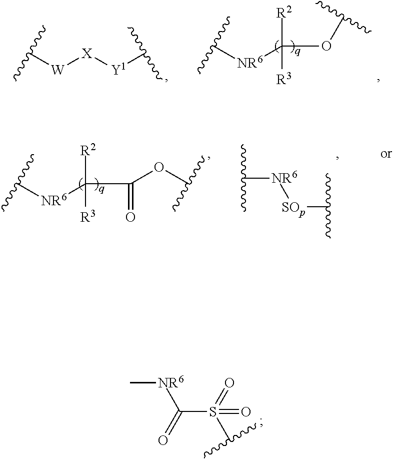

L² is selected from a single bond, a —O— linker, a

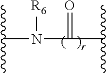

linker, a —C(O)— linker, a —CH₂— linker, a —CH₂O— linker, or a —CH═CH— linker;

R¹ is selected from hydrogen or alkyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments described herein of the compound of Formula (VI) or (VIa), rings in A are unsubstituted.

In some embodiments described herein of the compound of Formula (VI) or (VIa), rings in A can be substituted. In some such embodiments, A cam be substituted with one or more substituents selected from alkyl, amino, haloalkyl, halogen, hydroxy, alkoxy, haloalkoxy, cyano, or oxo. In some embodiments, A can be substituted with sulfonyl, for example, methanesulfonyl.

In some embodiments described herein of the compounds of Formula (VI), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments described herein of the compounds of Formula (VI), E can be unsubstituted or substituted. In some embodiments, E can be substituted with with one or more substituents selected from alkyl, amino, haloalkyl, halogen, hydroxy, alkoxy, haloalkoxy, cyano, or oxo. In some embodiments, E can be substituted with sulfonyl, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo; or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; halogen or cyano.

In some embodiments, C is selected from

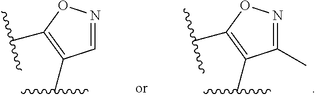

In some embodiments, C is selected from

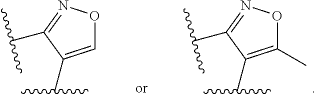

In some embodiments, C is selected from

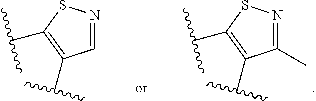

In some embodiments, C is selected from

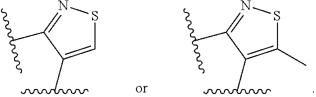

In some embodiments, C is selected from

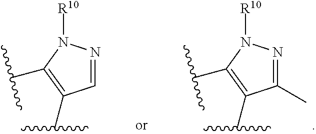

In some such embodiments, R¹⁰ is hydrogen. In some such embodiments, R¹⁰ is C₁₋₃ alkyl or C₃₋₆ cycloalkyl.

In some embodiments, C is

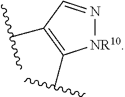

In some embodiments, C is

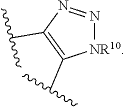

In some embodiments, C is selected from

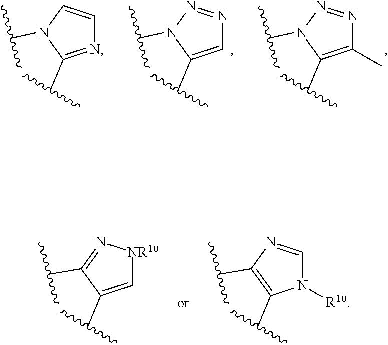

In some such embodiments, R¹⁰ is C₁₋₃ alkyl or C₃₋₆ cycloalkyl. In some such embodiments, R¹⁰ is hydrogen.

In some embodiments, C is selected from

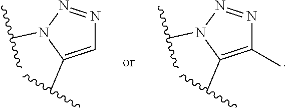

In some embodiments, C is selected from

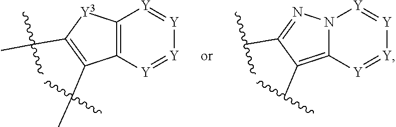

wherein Y³ is selected from O or S. In some such embodiments, Y is a CR⁶. In some other such embodiments, at least one Y is nitrogen. In some embodiments, C is optionally substituted

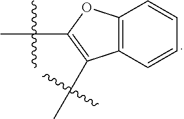 [SMILES: *c1oc2ccccc2c1*]

In some embodiments, C is selected from

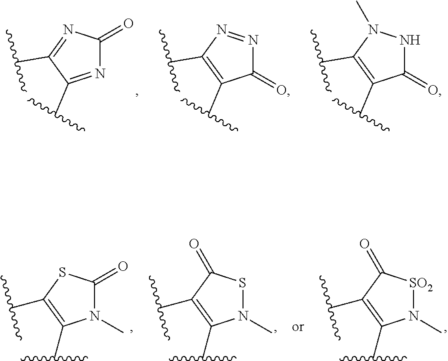

In some embodiments, m is 0. In some other embodiments, m is 1.

In some embodiments, each of R² and R³ is hydrogen. In some embodiments, a least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a —CH═CH— linker. In some other embodiments, L⁵ is a —C≡C— linker.

In some embodiments, L² is a single bond.

In some embodiments, R¹ is hydrogen. In some embodiments, R⁶ is hydrogen.

In some embodiments, R⁴ is selected from hydrogen or alkyl optionally substituted with halogen. In some other embodiments, R⁴ is unsubstituted alkyl

In some embodiments of the compound of Formula (VI) or (VIa),

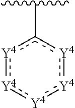

can be

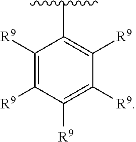

In some such embodiments, each of R⁹ is hydrogen. In some other such embodiments, at least one R⁹ is selected from C₁₋₃ alkyl or halogen.

In some embodiments,

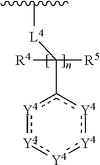

can be

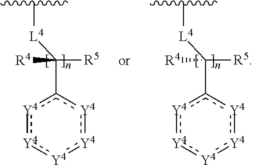

Some embodiments of the compounds of Formula (VI) are selected from compounds of Table 6 as shown below, and pharmaceutically acceptable salt thereof.

Some embodiments of the compounds of Formula (VI) are selected from compound IT013, as shown in Table 13.

Formula (VII)

Some embodiments disclosed herein include a compound of Formula (VII) as described above or a pharmaceutically acceptable salt thereof.

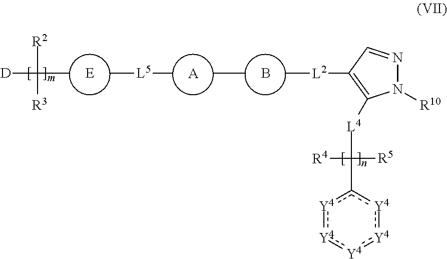

In some embodiments, one of A or B is an acetylene and the other one of A or B is selected from the group consisting of

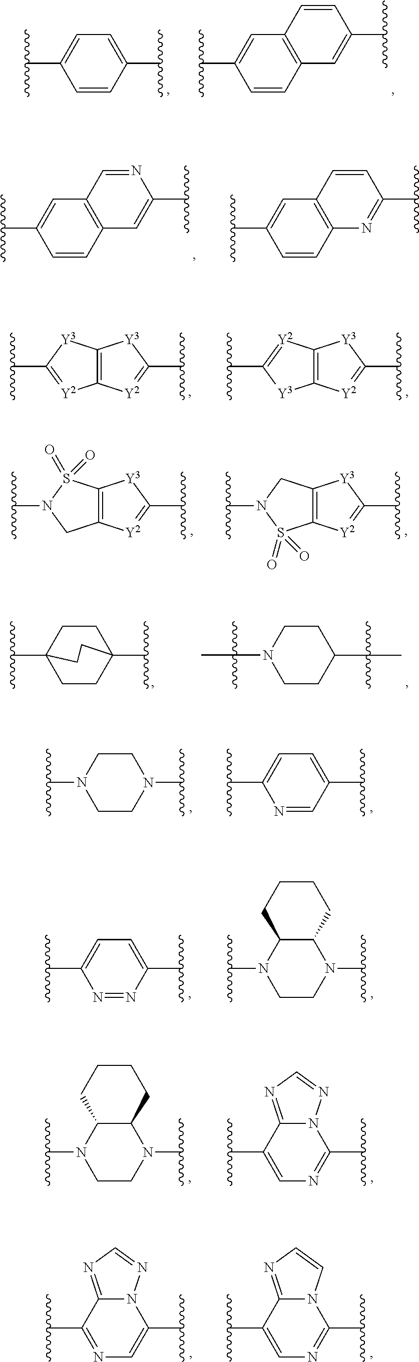

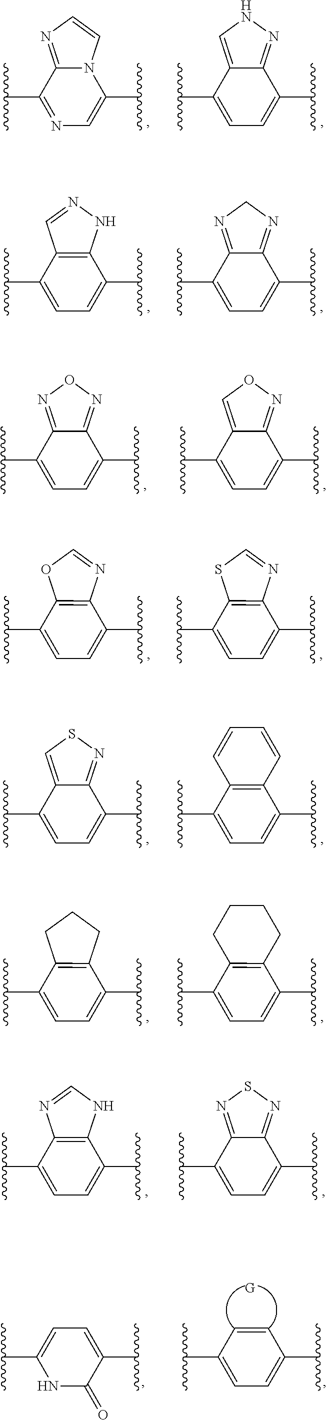

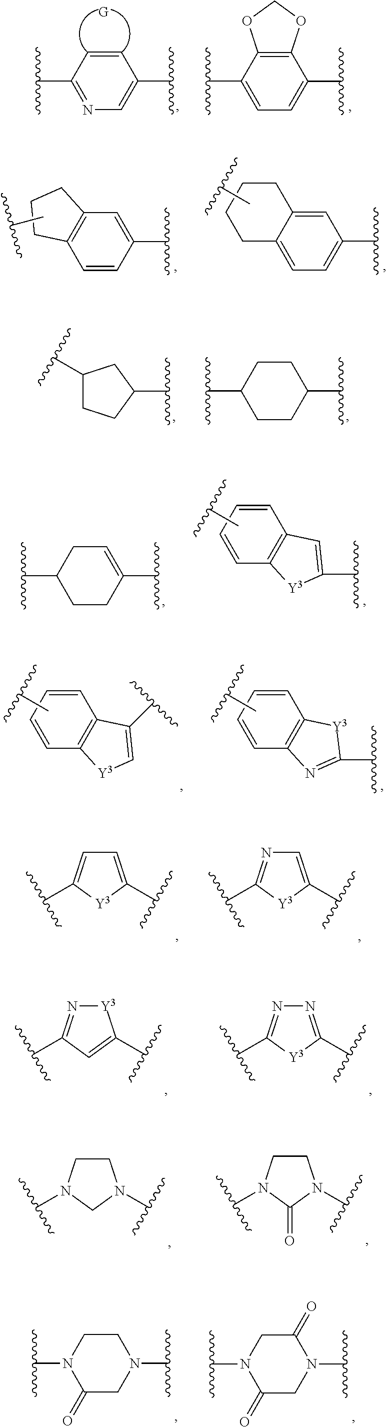

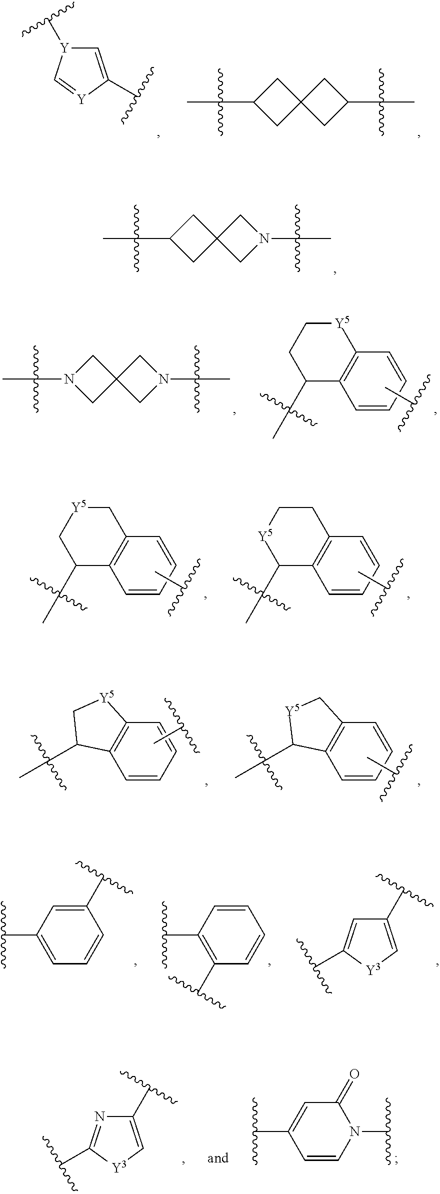

wherein the rings in A or B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

each Y is independently selected from CR⁶ or N; each Y² is independently selected from —CH═ or N; each Y³ is independently is selected from C(R⁶)₂, NR⁶, O or S; each Y⁵ is independently is selected from NR⁶, O or S.

In some embodiments, the compound of Formula (VII) is also represented by Formula (VIIa):

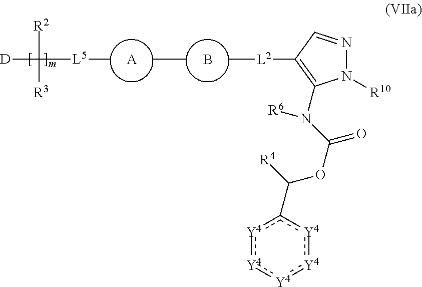

wherein one of A or B is an acetylene and the other one of A or B is selected from

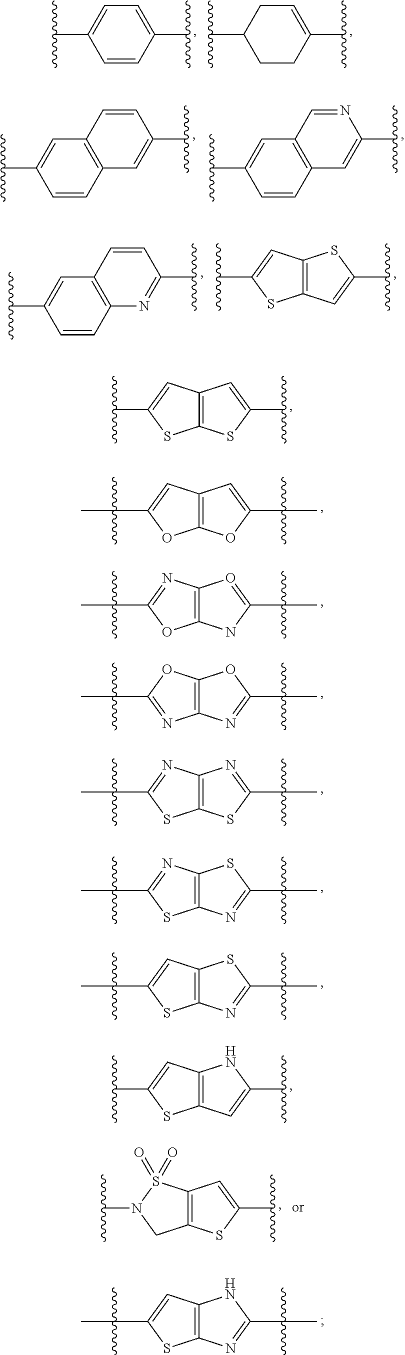

and wherein the rings in A or B are unsubstituted or substituted with one or more substituents selected from alkyl, amino, haloalkyl, halogen, hydroxy, alkoxy, haloalkoxy, cyano, or oxo.

In some such embodiments, A is acetylene and B is phenyl. In some such embodiments, A is acetylene and B is naphthyl. In some such embodiments, A is acetylene and B is selected from

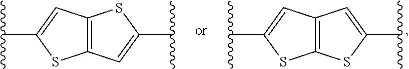 [SMILES: *c1cc2sc(*)cc2s1 ; *c1cc2cc(*)sc2s1]

each optionally substituted. In some such embodiments. A is acetylene and B is selected from

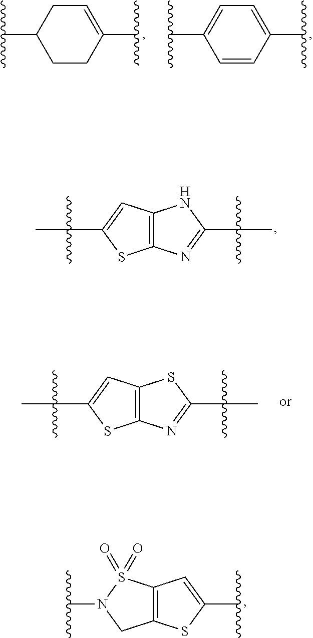

each optionally substituted. In some such embodiments, B is acetylene and A is phenyl. In some such embodiments, B is acetylene and A is naphthyl. In some such embodiments, B is acetylene and A is selected from

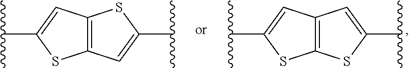 [SMILES: *c1cc2sc(*)cc2s1 ; *c1cc2cc(*)sc2s1]

each ontionally substituted. In some such embodiments, B is acetylene and A is selected from

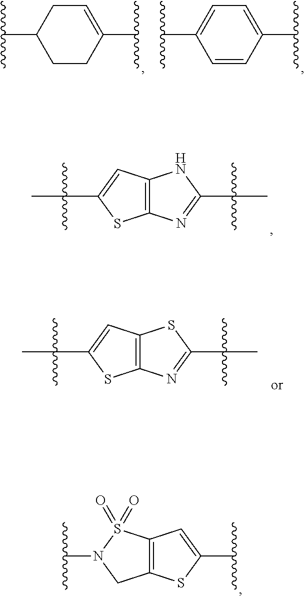

each optionally substituted. In one embodiment, A is optionally substituted

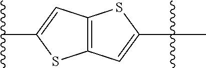 [SMILES: *c1cc2sc(*)cc2s1]

and B is acetylene. In another embodiment, A is optionally substituted

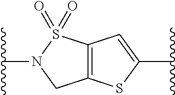 [SMILES: *c1cc2c(s1)CN(*)S2(=O)=O]

and B is acetylene.

In some alternative embodiments, L² is —(CH₂)₂—; B is absent; and A is selected from

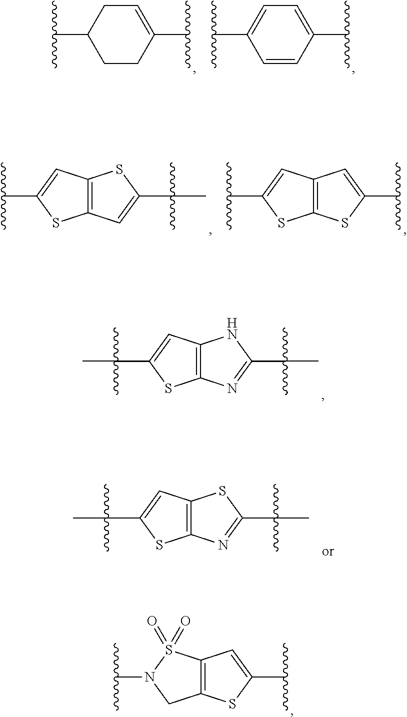

each optionally substituted with one or more substituents selected from the group consisting of alkyl, alkoxy, halogen, haloalkyl and cyano.

In some embodiments, each A, B or G are independently unsubstituted or unsubstituted or substituted with one or more substituents selected from alkyl, haloalkyl, halogen, hydroxy, alkoxy, haloalkoxy, cyano, or oxo:

D is selected from

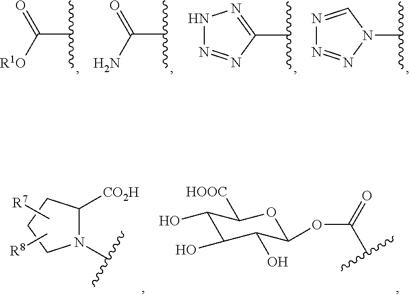

or carboxylic acid isosteres; E is absent; L² is selected from a single bond, a —O— linker, a

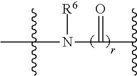

linker, a —C(O)— linker, a —CH₂— linker, a —CH₂O— linker, a —C≡C— linker, or a —CH═CH— linker; L⁴ is selected from

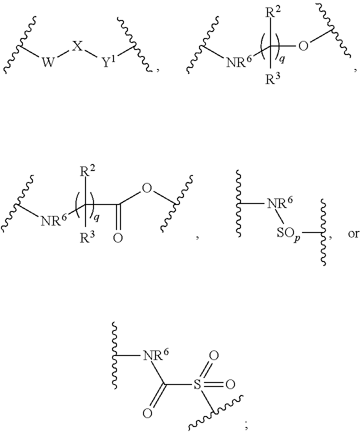

L⁵ is selected from a single bond, a —CH₂O— linker, a —CH═CH— linker, a —C≡C— linker,

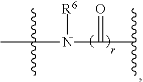

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyleach R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; and r is an integer of 0 or 1.

In some embodiments, the compound of Formula (VII) is also represented by Formula (VIIb):

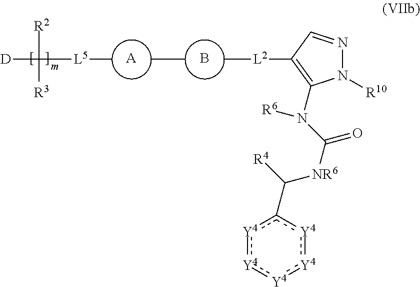

wherein one of A or B is an acetylene and the other one of A or B is selected from

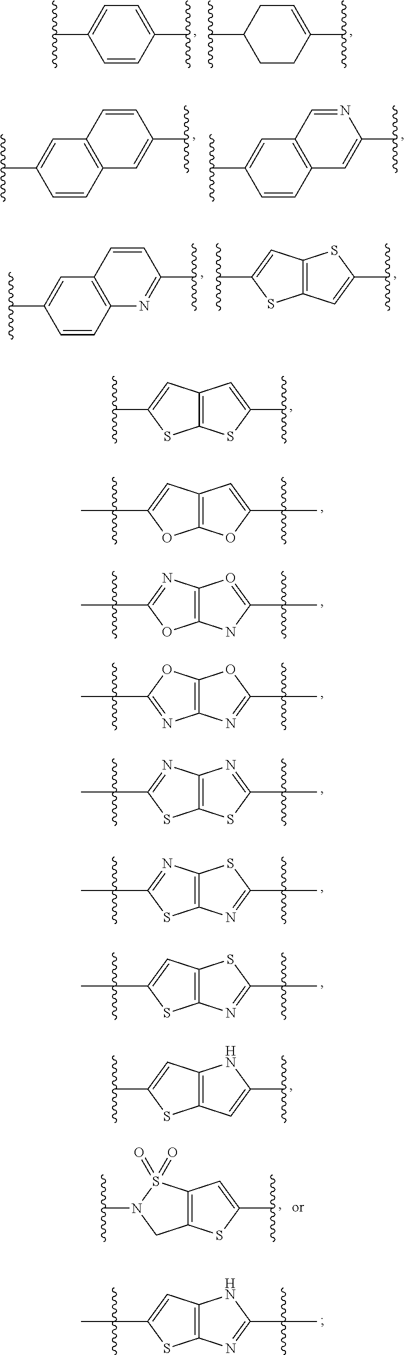

and wherein the rings in A or B are unsubstituted or substituted with one or more substituents selected from alkyl, amino, haloalkyl, halogen, hydroxy, alkoxy, haloalkoxy, cyano, or oxo.

In some such embodiments, A is acetylene and B is phenyl. In some such embodiments, A is acetylene and B is naphthyl. In some such embodiments, A is acetylene and B is selected from

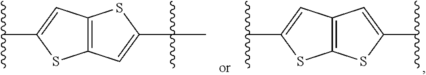 [SMILES: *c1cc2sc(*)cc2s1 ; *c1cc2cc(*)sc2s1]

each optionally substituted. In some such embodiments, A is acetylene and B is selected from

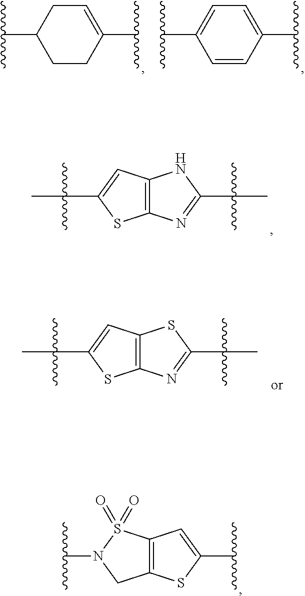

each optionally substituted. In some such embodiments, B is acetylene and A is phenyl. In some such embodiments, B is acetylene and A is naphthyl. In some such embodiments, B is acetylene and A is selected from

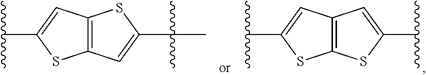 [SMILES: *c1cc2sc(*)cc2s1 ; *c1cc2cc(*)sc2s1]

each optionally substituted. In some such embodiments, B is acetylene and A is selected from

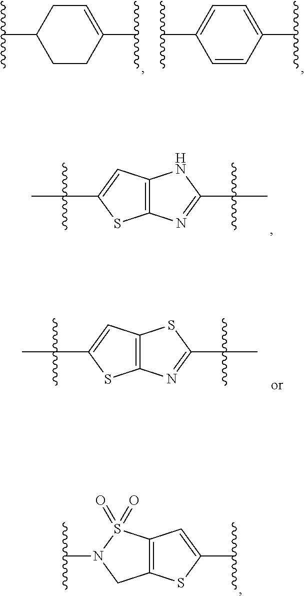

each optionally substituted. In one embodiment, A is optionally substituted

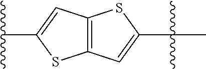 [SMILES: *c1cc2sc(*)cc2s1]

and B is acetylene. In another embodiment, A is optionally substituted

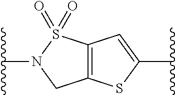 [SMILES: *c1cc2c(s1)CN(*)S2(=O)=O]

and B is acetylene.

In some embodiments, the compound of Formula (VII) is also represented by Formula (VIIc):

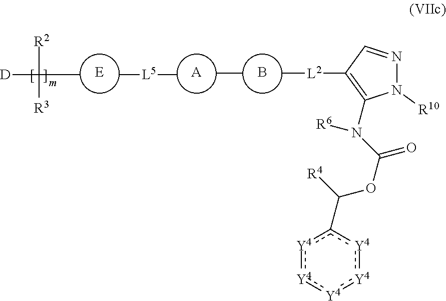

wherein one of A or B is an acetylene and the other A or B is selected from

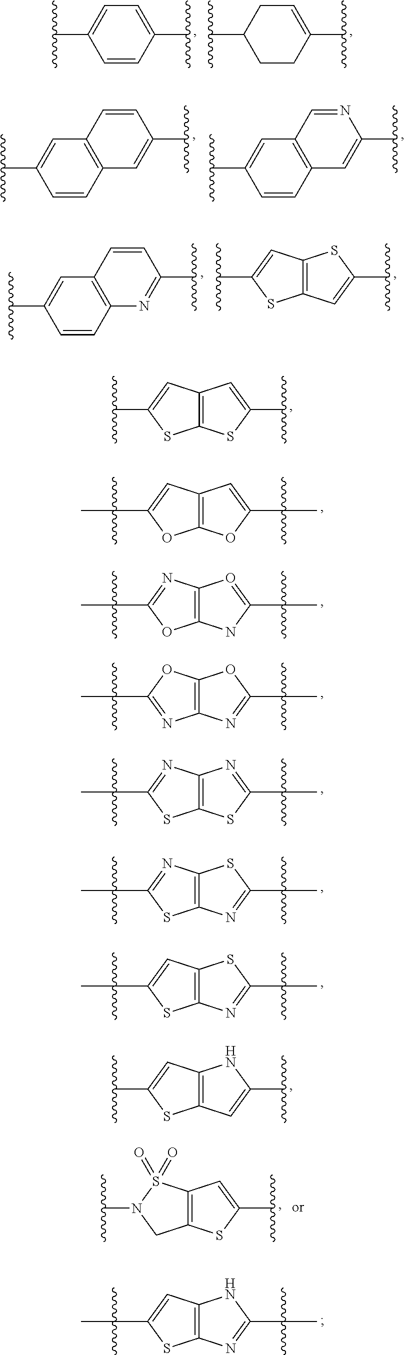

and wherein the rings in A or B are unsubstituted or substituted with one or more substituents selected from alkyl, amino, haloalkyl, halogen, hydroxy, alkoxy, haloalkoxy, cyano, sulfonyl or oxo; E is selected from optionally substituted phenylene, or optionally substituted 5 to 6 membered heteroarylene. In some such embodiments, E is selected from

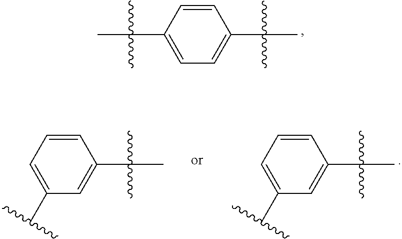 [SMILES: *c1ccc(*)cc1 ; *c1cccc(*)c1 ; *c1cccc(*)c1]

In some embodiments described herein of the compound of Formula (VII), (VIla), (VIIb) or (VIIc), rings in A or B are unsubstituted.

In some embodiments described herein of the compound of Formula (VII), (VIIa), (VIIb) or (VIIc), rings in A or B can be substituted. In some embodiments, rings in A or B can be substituted with one or more substituents selected from alkyl, amino, haloalkyl, halogen, hydroxy, alkoxy, haloalkoxy, cyano, or oxo. In some further embodiments, rings in A or B can be substituted with one or more substituents selected from alkyl, haloalkyl, halogen or alkoxy. In some other embodiments, rings in A or B can be substituted with sulfonyl, for example, methanesulfonyl.

In some embodiments described herein of the compound of Formula (VII), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some such embodiments, E can be substituted with one or more substituents selected from alkyl, amino, haloalkyl, halogen, hydroxy, alkoxy, haloalkoxy, cyano, or oxo. In some other embodiments, rings in E can be substituted with sulfonyl, for example, methanesulfonyl.

In some embodiments, R¹⁰ is C₁₋₃ alkyl. In some other embodiments, R¹⁰ is C₃₋₆ cycloalkyl.

In some embodiments, D is selected from —OH,

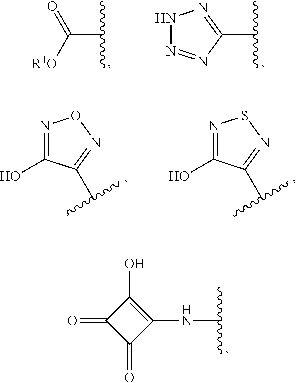

—NHS(O)₂R¹⁴, or —C(O)—NHS(O)₂R¹⁴. In some such embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² and R³ is hydrogen and the other R² and R³ is aryl. In some other embodiments, one of R² and R³ is hydrogen and the other R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some other embodiments, L⁵ is —C(O)NR^{6b}, wherein R^{6b} is hydrogen or C₁₋₃ alkyl.

In some embodiments, L² is a single bond.

In some embodiments, R⁶ is hydrogen. In some other embodiments, R⁶ is C₁₋₃ alkyl.

In some embodiments, R⁴ is alkyl optionally substituted with halogen. In some other embodiments, R⁴ is hydrogen.

In some embodiments of the compound of Formula (VII) or (VIIa),

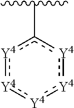

can be

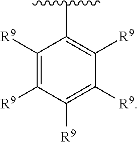

In some embodiments, each of R⁹ is hydrogen. In some other embodiments, at least one R⁹ is selected from C₁₋₃ alkyl or halogen.

In some embodiments,

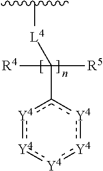

can be

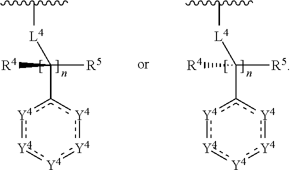

Some embodiments of the compounds of Formula (VII) are selected from compounds of Tables 7A, 7B, 7C and 7D as shown below, and pharmaceutically acceptable salt thereof.

Some embodiments of the compounds of Formula (VII) are selected from the group consisting of compounds IT014-IT018, IT070, IT082-IT090, IT092, IT095, IT097-IT100, IT103, IT104, IT107, IT109, IT110, IT114, IT118, IT126, IT127, IT371, IT398-IT405, IT429-IT432, IT466, and IT479 as shown in Table 13.

Formula (VIII)

Some embodiments disclosed herein include a compound of Formula (VIII) as described above or a pharmaceutically acceptable salt thereof.

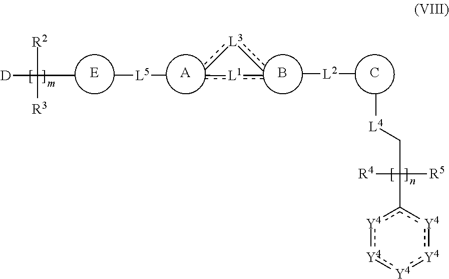

In some embodiments, each of A and B is selected from the group consisting of

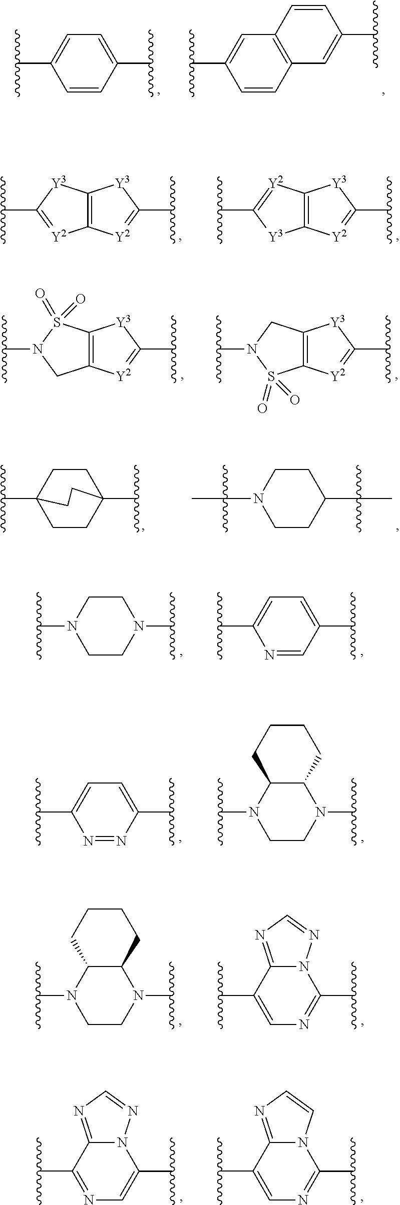

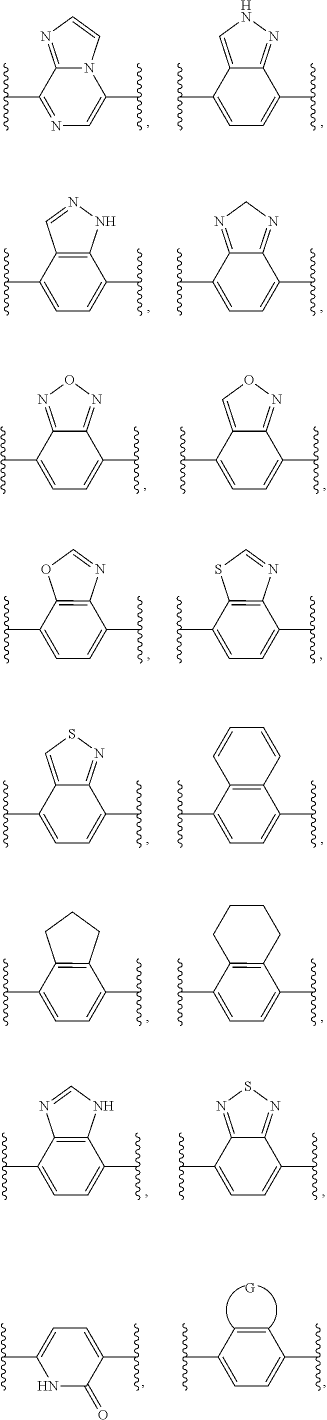

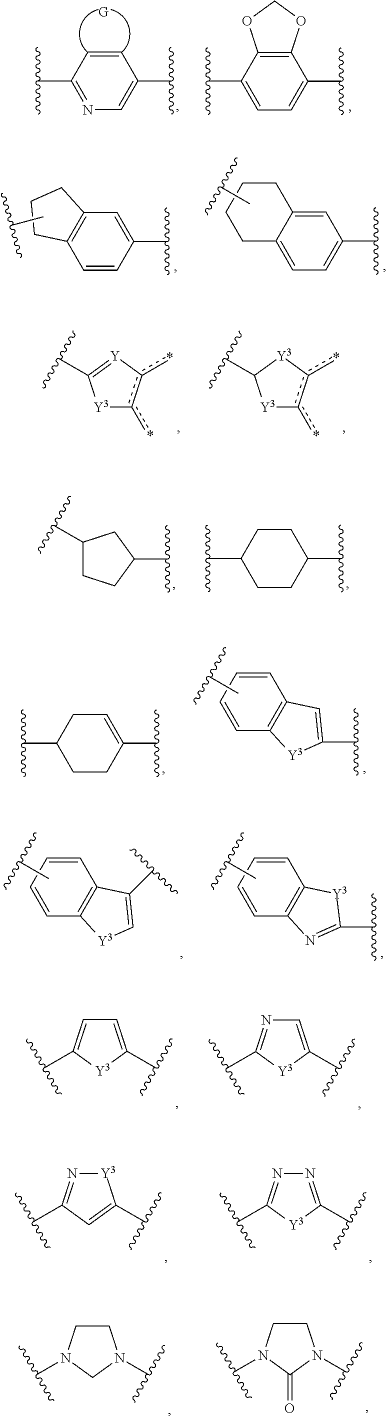

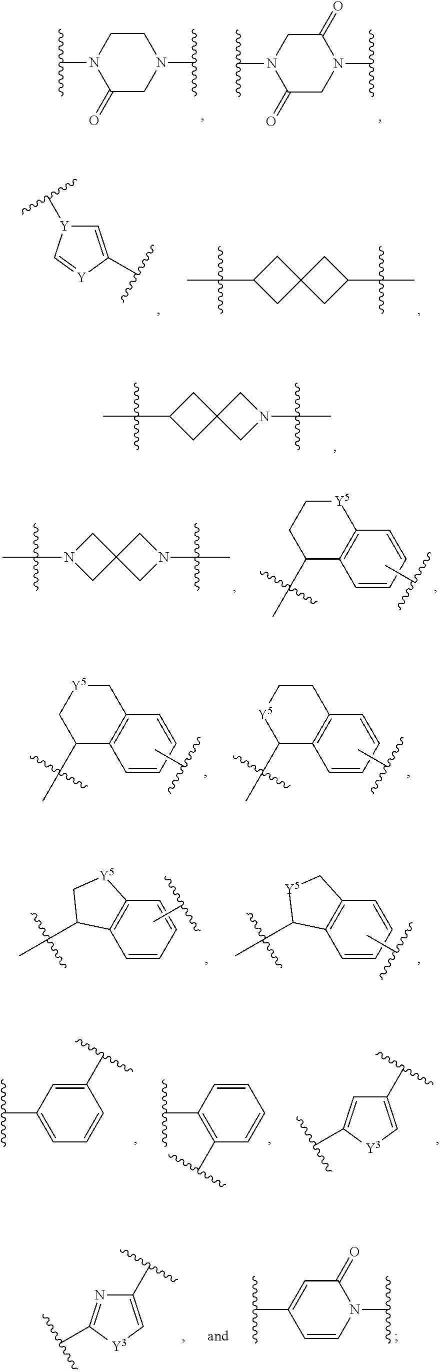

wherein each * is a point of attachment of A or B to L¹ or L³, and wherein the rings in A and B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo; and each Y⁵ is independently selected from NR⁶, O or S.

In some embodiments, the compound of Formula (VIII) is also represented by Formula (VIIIa):

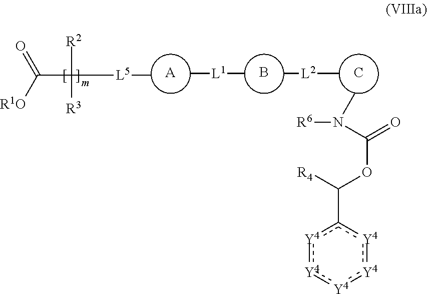

wherein one of A or B is phenyl and the other one of A or B is selected from

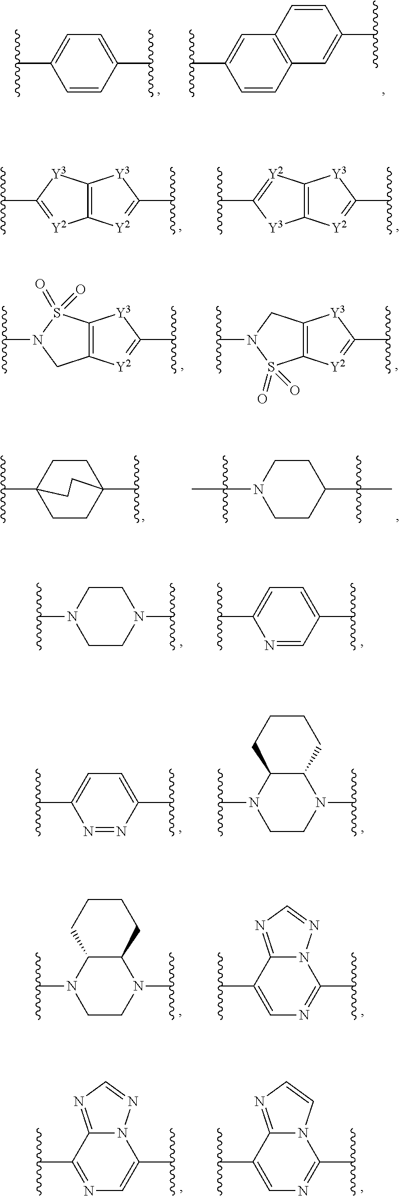

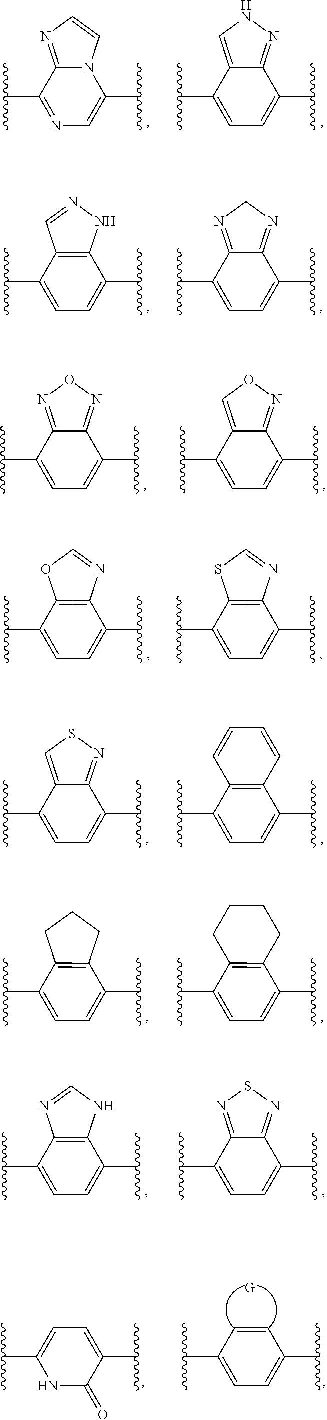

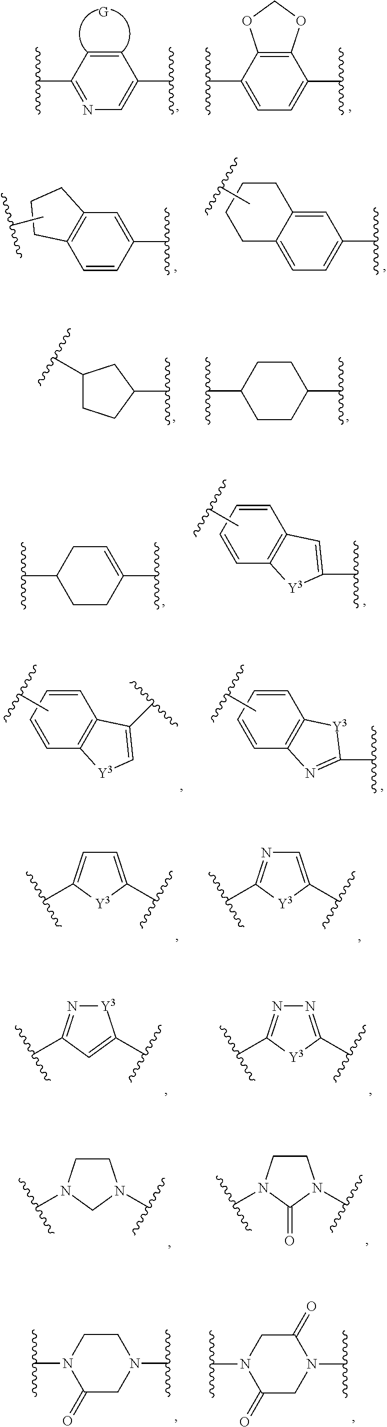

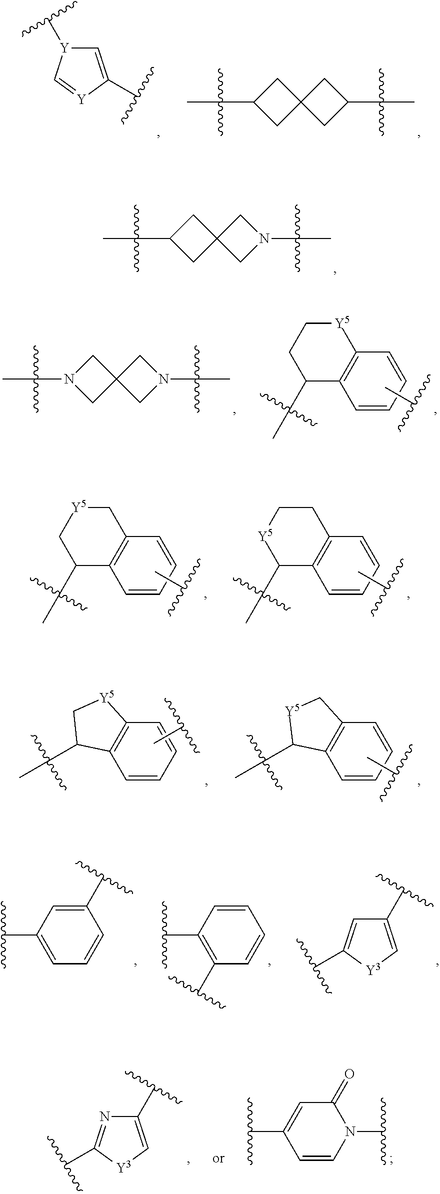

wherein each of A and B is unsubstituted or substituted with one or more substituents selected from alkyl, haloalkyl, amino, halogen, hydroxy, alkoxy, haloalkoxy, cyano, or oxo. In some such embodiments, both A and B are phenyl. In some such embodiments, both A and B are unsubstituted phenyl.

In some embodiments, each of A, B and G is independently unsubstituted or substituted with one or more substituents selected from alkyl, haloalkyl, halogen, hydroxy, alkoxy, haloalkoxy, cyano, or oxo;

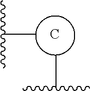

is a ring system selected from the group consisting of

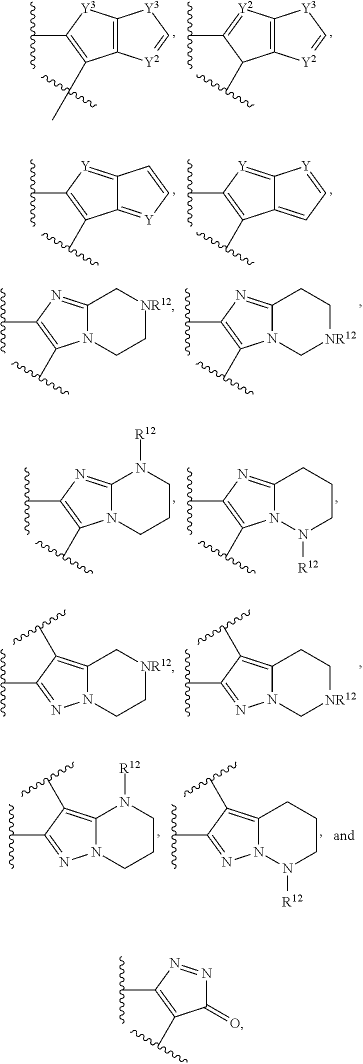

wherein C is optionally substituted; D is selected from

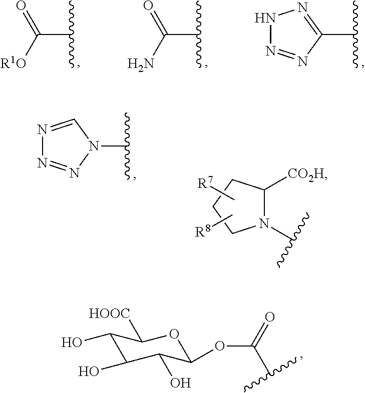

or carboxylic acid isosteres; E is absent; L⁴ is selected from

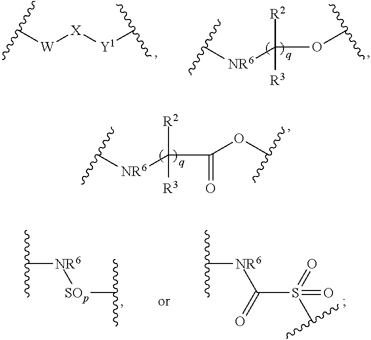

L¹ is selected from a single bond, a —O— linker, a

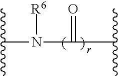

linker, a —C(O)— linker, a CH₂— linker, a —CH₂O— linker, a —C≡C— linker, a —CH═CH— linker, or a ═C(R¹¹)— linker; L² is selected from a single bond, a —O— linker, a

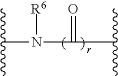

linker, a —C(O)— linker, a —CH₂— linker, a —CH₂O— linker, a —C≡C— linker, or a —CH═CH— linker; L⁵ is selected from a single bond, a —CH₂O-linker, a —CH═CH— linker, a —C≡C— linker,

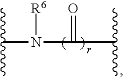

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments described herein of the compound of Formula (VIII) or (VIIIa), rings in A and B are unsubstituted.

In some embodiments described herein of the compound of Formula (VIII) or (VIIIa), rings in A and B can be substituted. In some such embodiments, rings in A and B can be substituted with one or more substituents selected from alkyl, haloalkyl, halogen, hydroxy, alkoxy, haloalkoxy, cyano, or oxo. In some other embodiments, rings in A or B can be substituted with sulfonyl, for example, methanesulfonyl.

In some embodiments described herein of the compound of Formula (VIII), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some other embodiments, E can be substituted with sulfonyl, for example, methanesulfonyl.

In some embodiments, C is substituted with one or more one or more substituents selected from C₁₋₃ alkyl, C₃₋₆ cycloalkyl, halogen, oxo or cyano. In some other embodiments, C is unsubstituted.

In some embodiments, C is selected from

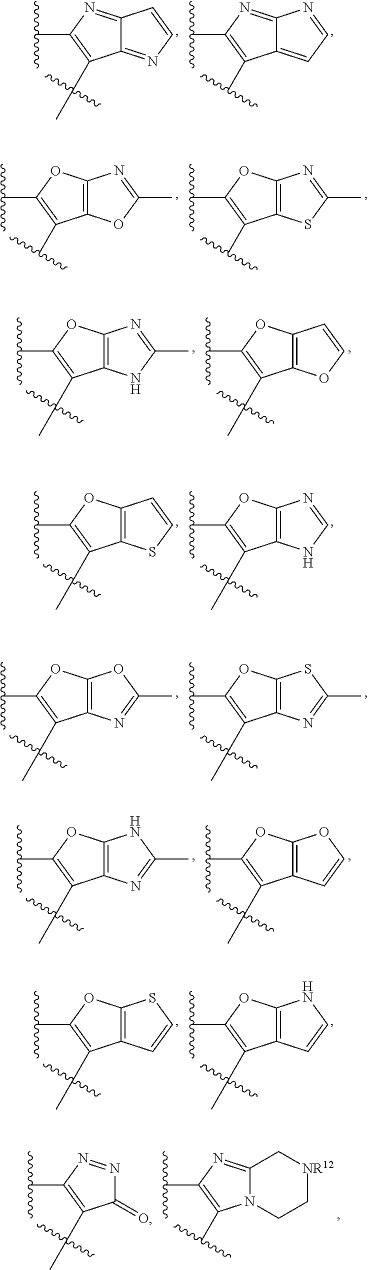

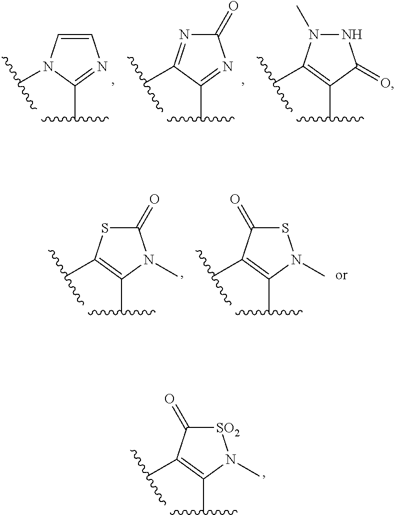

each optionally substituted with one or more substituents selected from the group consisting of C₁₋₃ alkyl optionally substituted with halogen or C₁₋₃ alkoxy; C₁₋₆ alkoxy; C₃₋₆ cycloalkyl; halogen; or cyano. In some such embodiment, C is

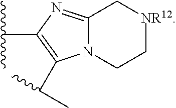

In some such embodiments, each R¹² is independently selected from hydrogen, C₁₋₃ alkyl, —C(O)CH₃, —S(O)₂CH₃, —C(O)NHCH₃, or —C(O)OC₂H₅. In some other such embodiment, C is selected from

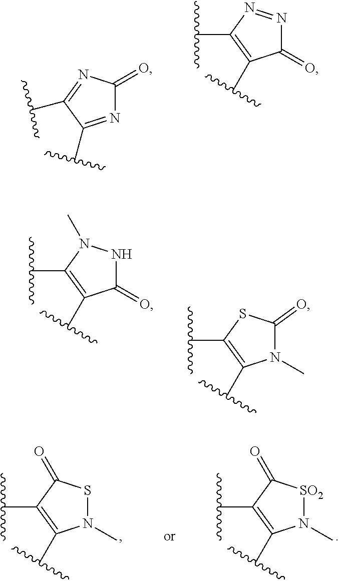

In some embodiments, m is 0. In some other embodiments, m is 1.

In some embodiments, each of R² and R³ is hydrogen. In some embodiments, at least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond. In some embodiments, L¹ is a single bond.

In some embodiments, R⁶ is hydrogen. In some embodiments, R¹ is hydrogen.

In some embodiments, R⁴ is alkyl optionally substituted with halogen. In some other embodiments, R⁴ is hydrogen.

In some embodiments of the compound of Formula (VIII) or (VIIIa),

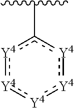

can be

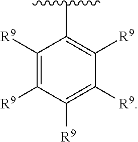

In some such embodiments, each of R⁹ is hydrogen. In some other such embodiments, at least one R⁹ is selected from C₁₋₃ alkyl or halogen.

In some embodiments,

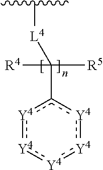

can be

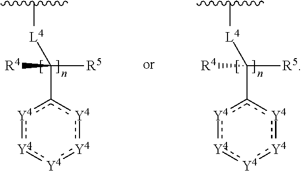

Some embodiments of the compounds of Formula (VIII) are selected from compounds of Table 8 as shown below, and pharmaceutically acceptable salt thereof.

Some embodiments of the compounds of Formula (VIII) are selected from compounds IT019-IT024, as shown in Table 13.

Formula (IX)

Some embodiments disclosed herein include a compound of Formula (IX) as described above or a pharmaceutically acceptable salt thereof.

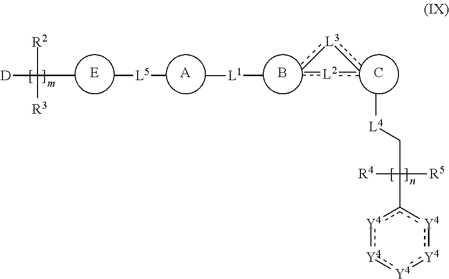

In some embodiments, A is acetylene or each of A and B is a ring system selected from

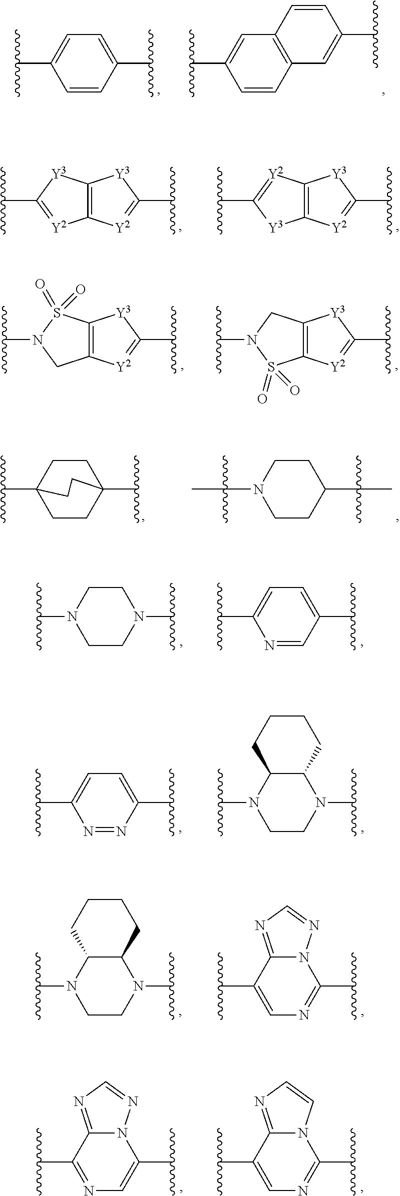

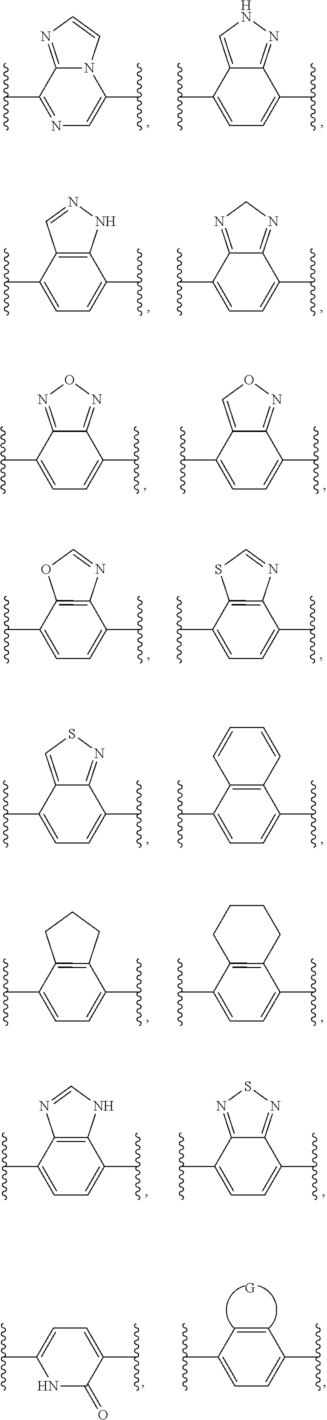

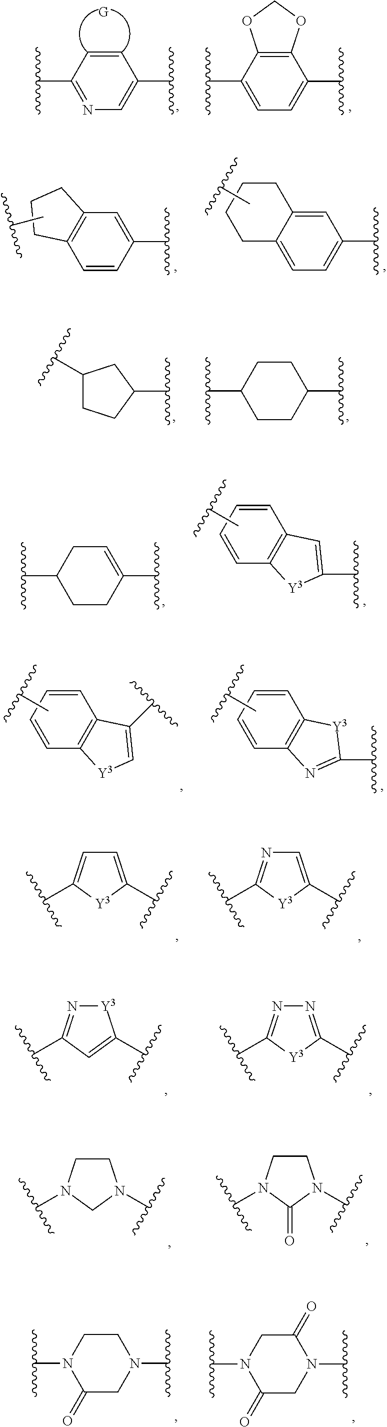

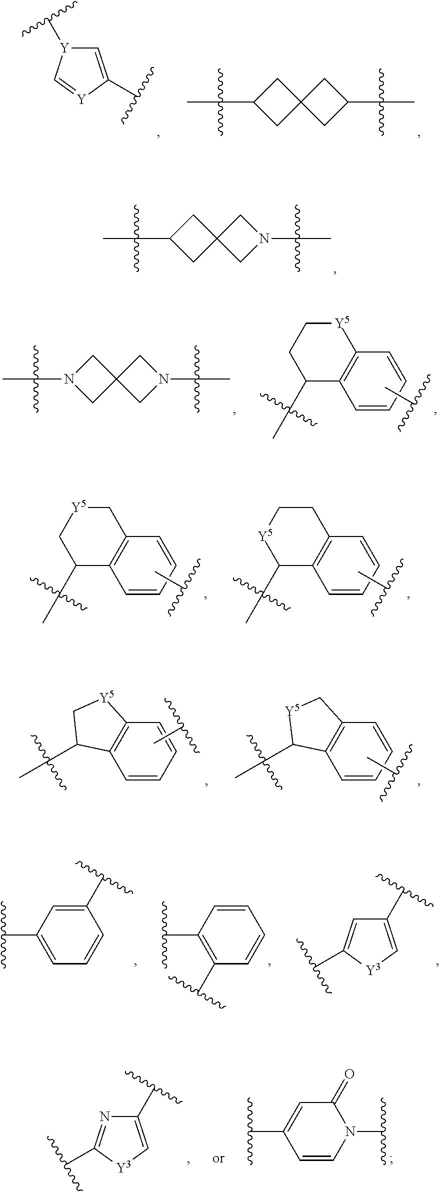

wherein each of A and B is unsubstituted or substituted with one or more substituents selected from alkyl, amino, haloalkyl, halogen, hydroxy, alkoxy, haloalkoxy, sulfonyl, cyano, or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

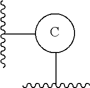

is selected from

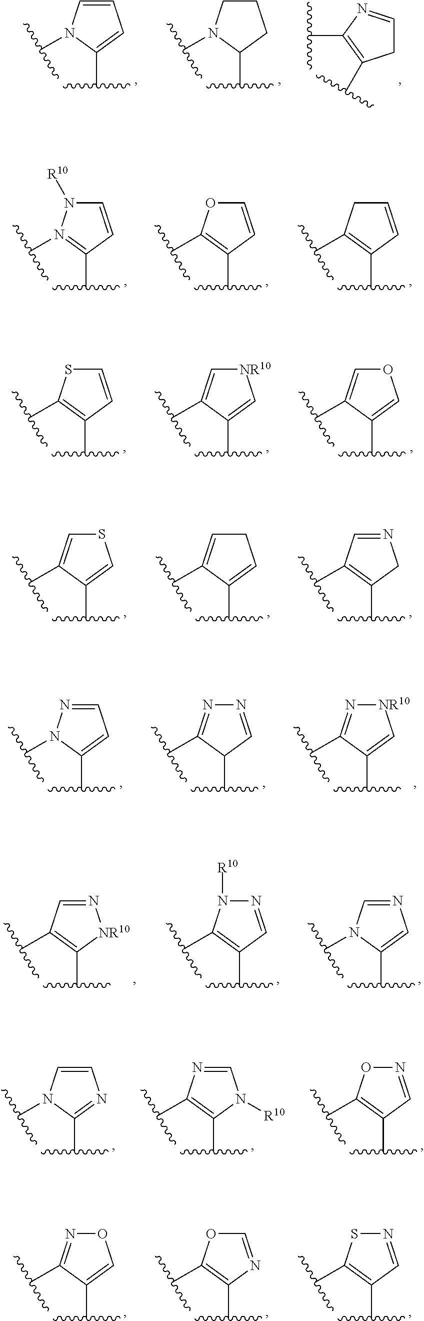

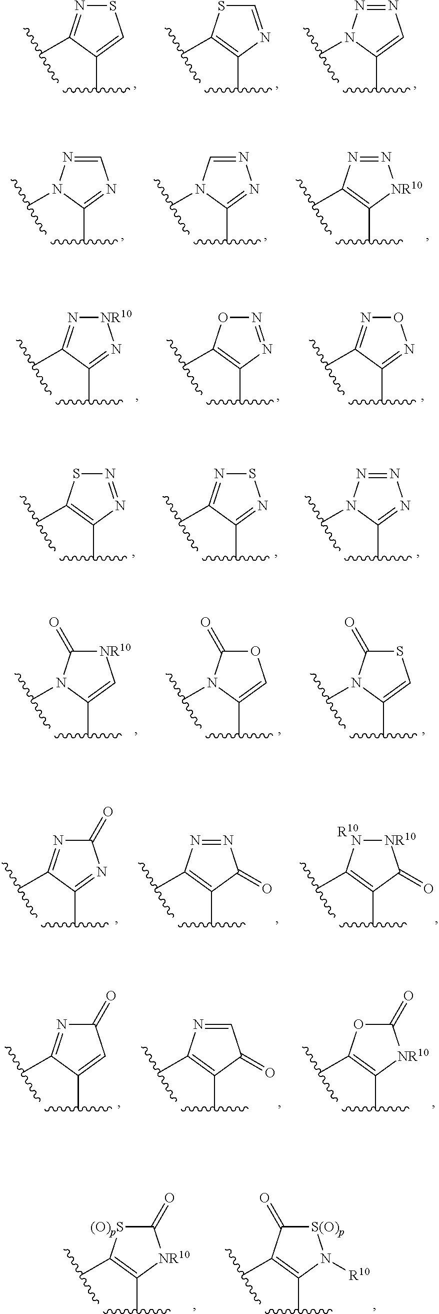

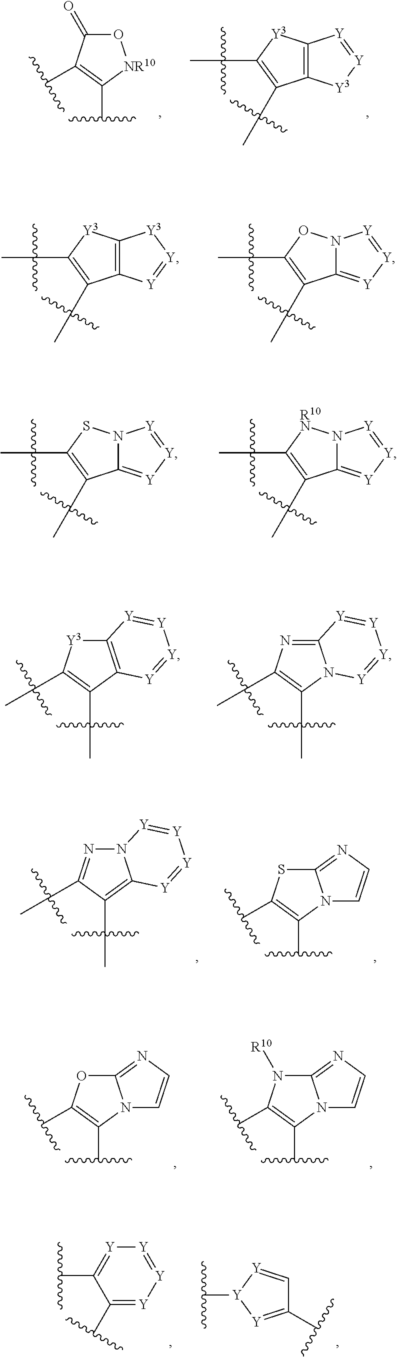

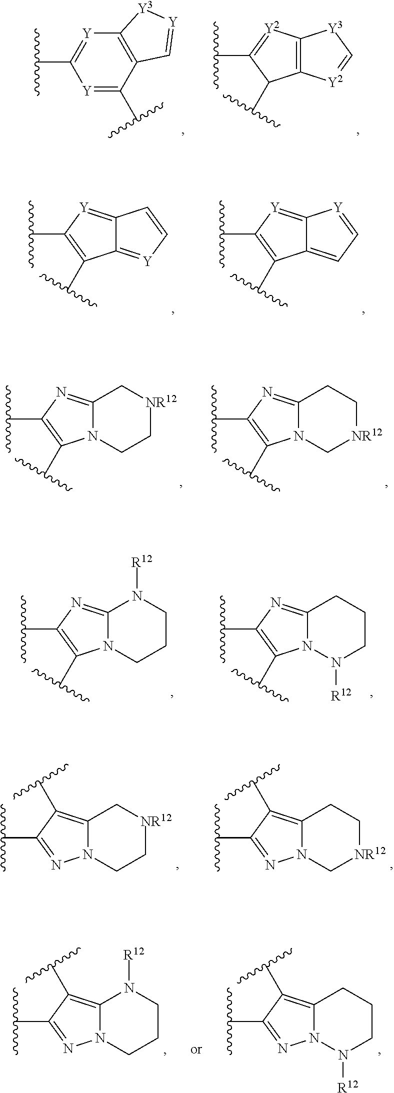

or optionally substituted variants thereof;

each Y² is independently selected from —CH═ or N; each Y³ is independently selected from C(R⁶)₂, NR⁶, O, or S; each Y⁵ is independently selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (IX) is also represented by Formula (IXa):

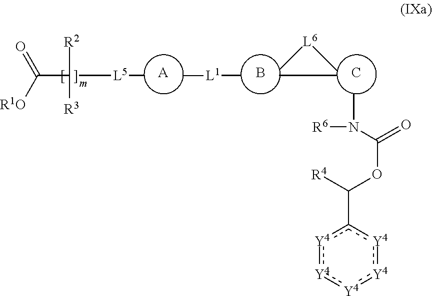

wherein B is phenyl; and A is selected from acetylene.

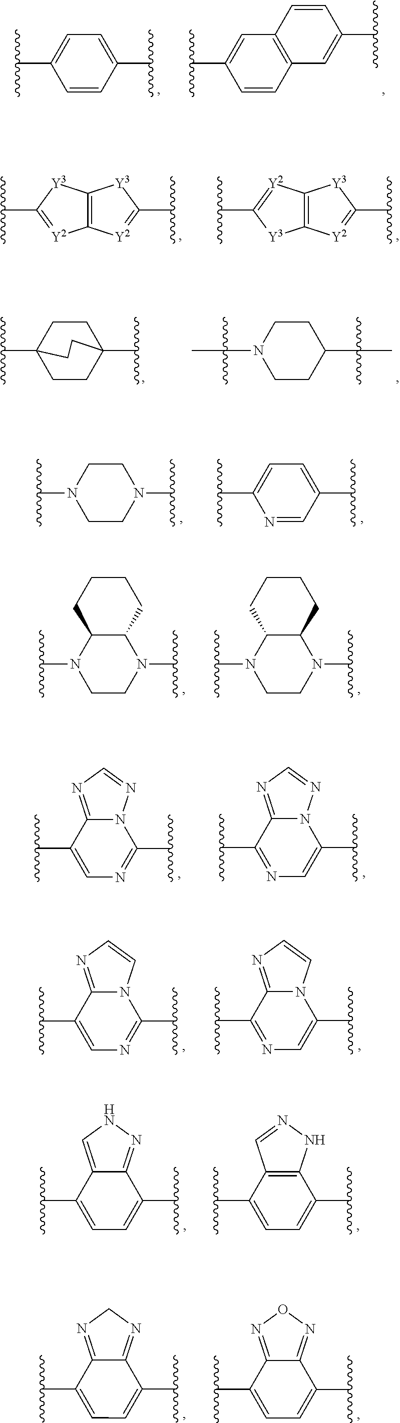

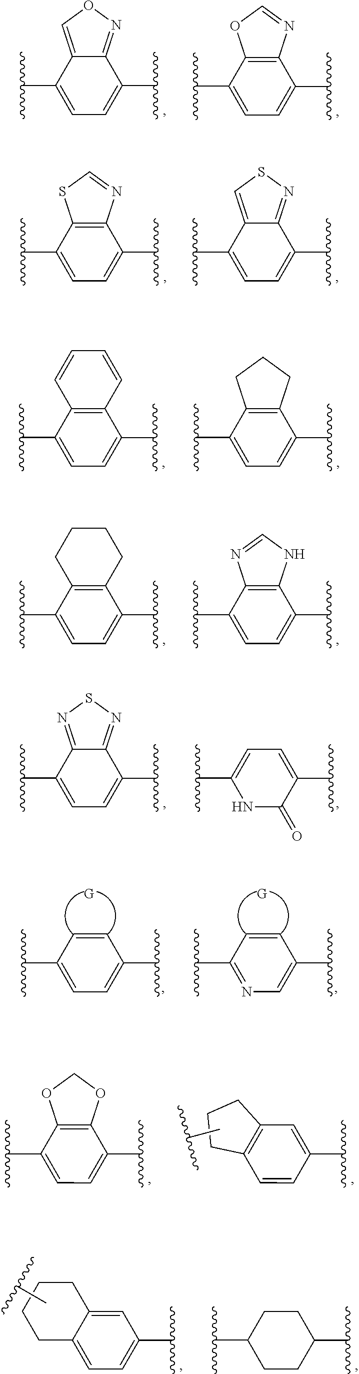

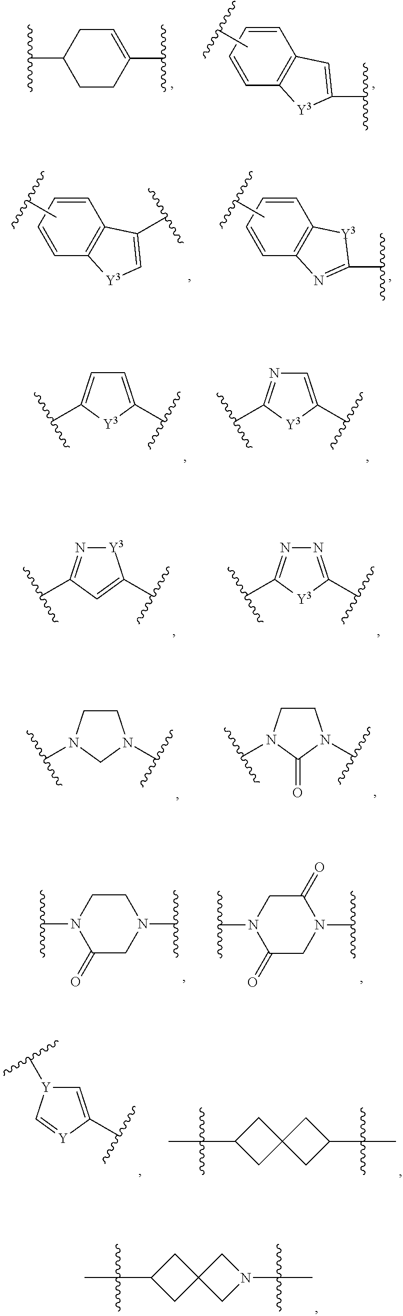

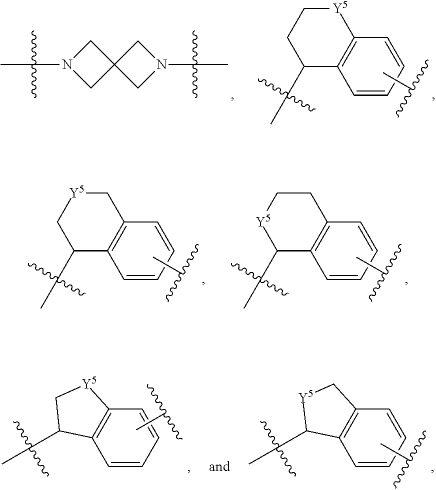

wherein each of the rings in A and B is unsubstituted or substituted with one or more substituents selected from alkyl, amino, haloalkyl, halogen, hydroxy, alkoxy, haloalkoxy, cyano, or oxo. In some of such embodiments, both A and B are phenyl. In some of such embodiments, A is acetylene and B is phenyl.

In some embodiments, each of A, B and G is unsubstituted or substituted with one or more substituents selected from alkyl, haloalkyl, halogen, hydroxy, alkoxy, haloalkoxy, cyano, or oxo;

D is selected from

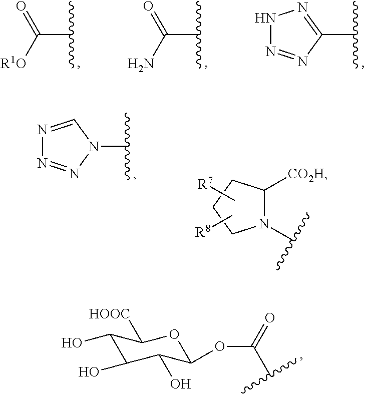

or carboxylic acid isosteres; E is absent; L⁴ is selected from

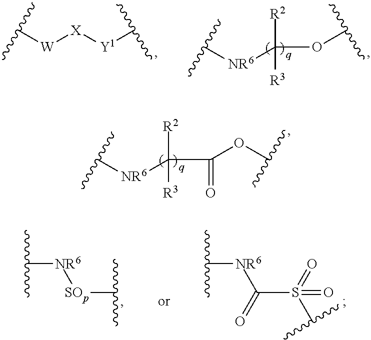

L¹ is selected from a single bond, a —O— linker, a —NH— linker, a —C(O)— linker, a —CH₂— linker, a —CH₂O— linker, a —C≡C— linker, or a —CH═CH— linker; L² is selected from a single bond, a —O— linker, a

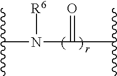

linker, a C(O)— linker, a —CH₂— linker, a —CH₂O— linker, a —C≡C— linker, a —CH═CH— linker, or a ═C(R¹¹)— linker; L⁵ is selected from a single bond, a —CH₂O— linker, a —CH═CH— linker, a —C≡C— linker,

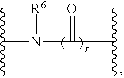

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments described herein of the compound of Formula (IX) or (IXa), rings in A and B are unsubstituted.

In some embodiments described herein of the compound of Formula (IX) or (IXa), rings in A and B can be substituted. In some embodiments, rings in A and B can be substituted with one or more substituents selected from alkyl, amino, haloalkyl, halogen, hydroxy, alkoxy, haloalkoxy, cyano, or oxo. In some embodiments, rings in A and B can be substituted with one or more sulfonyl groups, for example, methanesulfonyl.

In some embodiments described herein of the compound of Formula (IX), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl alkyl optionally substituted with halogen or C₁₋₃ alkoxy; C₁₋₆ alkoxy; C₃₋₆ cycloalkyl; halogen; oxo; or cyano. In some other embodiments, C is unsubstituted.

In some further embodiments, C is selected from an oxazole, an isoxazole, a thiazole, or an isothiazole, and wherein C is unsubstituted or substituted with one or more substituents selected from C₁₋₃ alkyl alkyl optionally substituted with halogen or C₁₋₃ alkoxy; C₁₋₆ alkoxy; C₃₋₆ cycloalkyl; halogen or cyano.

In some embodiments, C is selected from

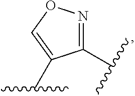 [SMILES: *c1conc1*]

In some embodiments, C is selected from

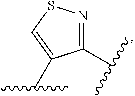 [SMILES: *c1csnc1*]

In some embodiments, C is

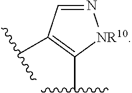

In some such embodiments, R¹⁰ is hydrogen. In some other such embodiments, R¹⁰ is C₁₋₃ alkyl or C₃₋₆ cycloalkyl.

In some embodiments, m is 0. In some other embodiments, m is 1.

In some embodiments, each of R² and R³ is hydrogen. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond. In some embodiments, L¹ is a single bond.

In some embodiments, L⁶ is

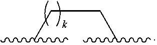

In some such embodiments, k is 0. In some other such embodiments, k is 1.

In some embodiments, L⁶ is

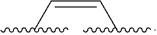 [SMILES: */C=C\*]

In some embodiments, R⁶ is hydrogen. In some embodiments, R¹ is hydrogen.

In some embodiments, R⁴ is alkyl.

In some embodiments of the compound of Formula (IX) or (IXa),

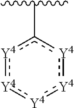

can be

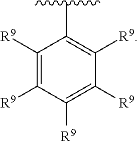

In some such embodiments, each of R⁹ is hydrogen. In some other such embodiments, at least one R⁹ is selected from C₁₋₃ alkyl or halogen.

In some embodiments,

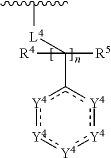

can be

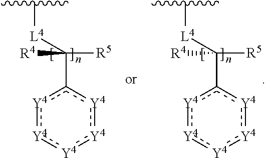

Some embodiments of the compound of Formula (IX) are selected from compounds of Table 9 as shown below, and pharmaceutically acceptable salt thereof.

Formula (X)

Some embodiments disclosed herein include a compound of Formula (X) as described above or a pharmaceutically acceptable salt thereof.

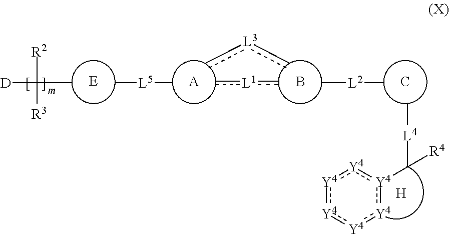

In some embodiments, each of A and B is an acetylene or selected from the group consisting of

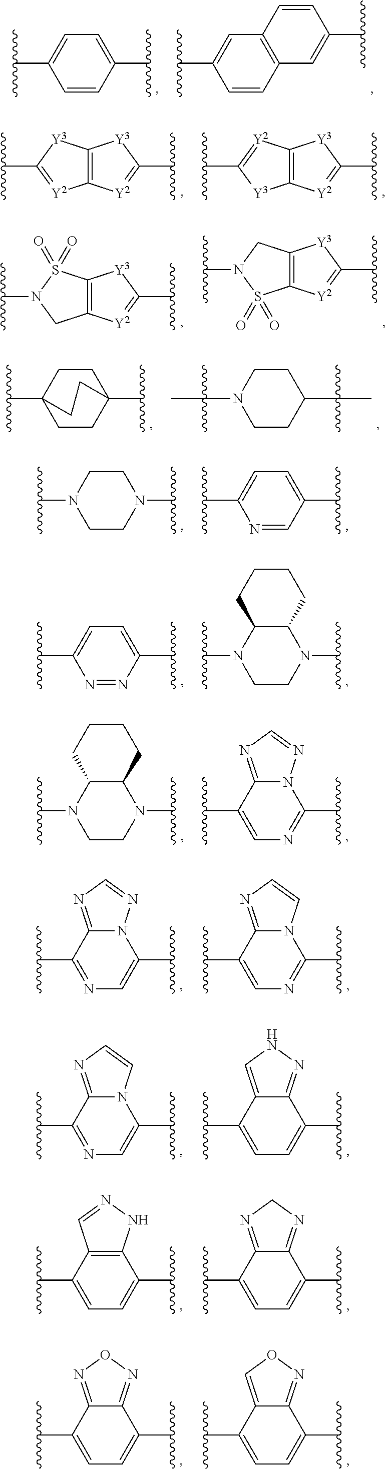

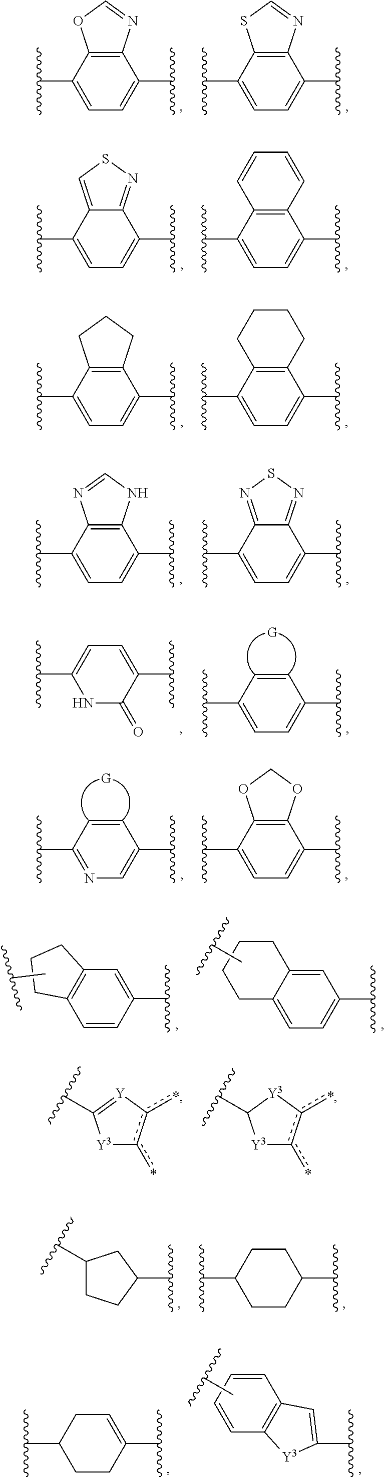

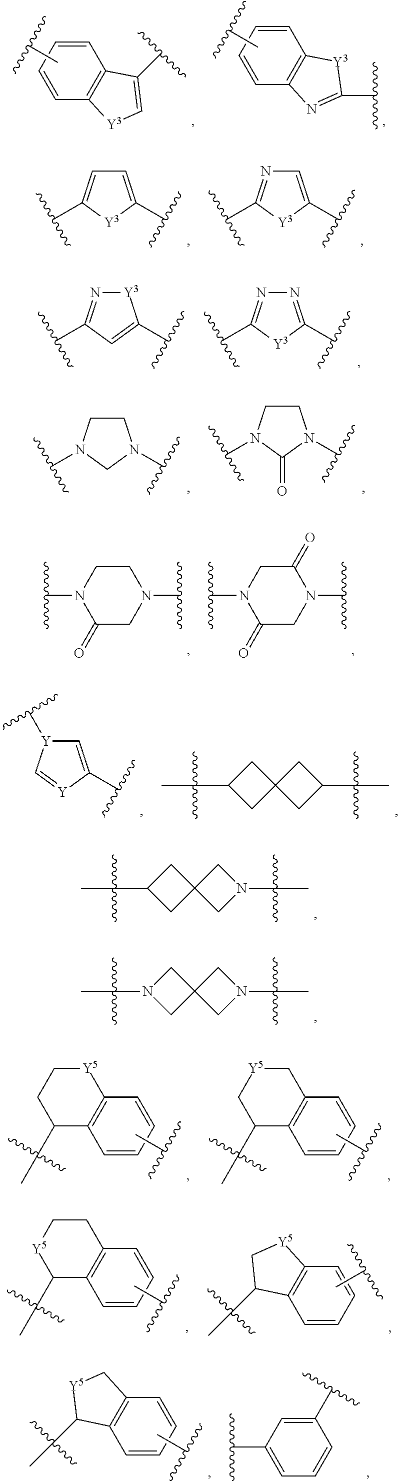

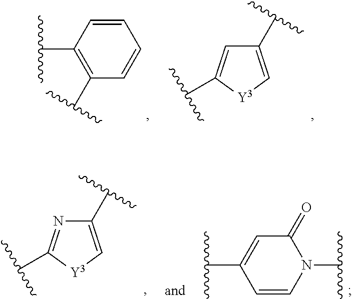

wherein each * is a point of attachment of A or B to L¹ or L³, and wherein the rings in A and B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, hydroxy, alkoxy, haloalkoxy, cyano, or oxo;

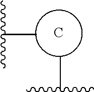

is selected from

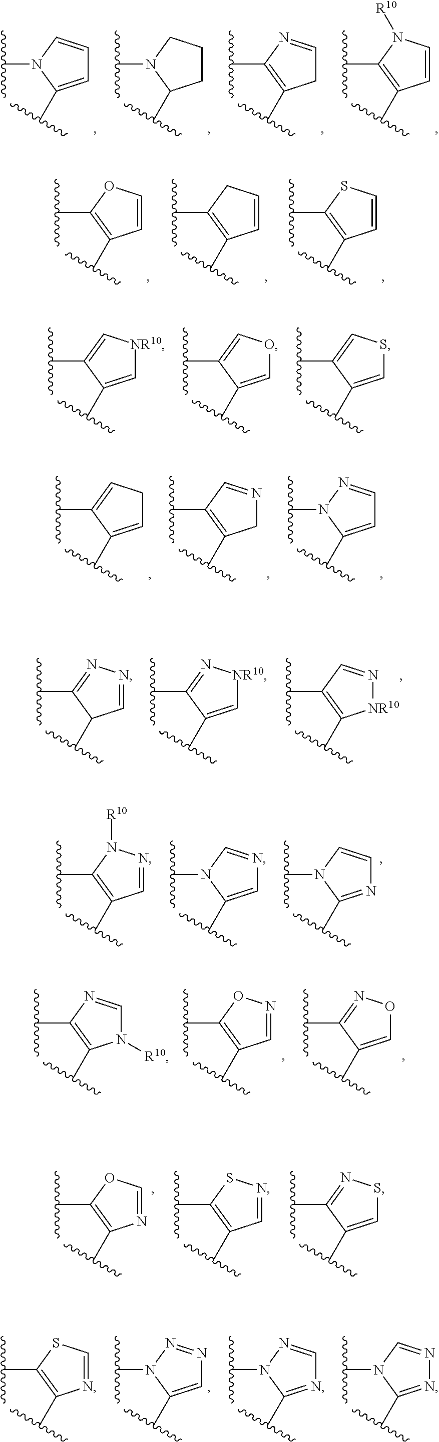

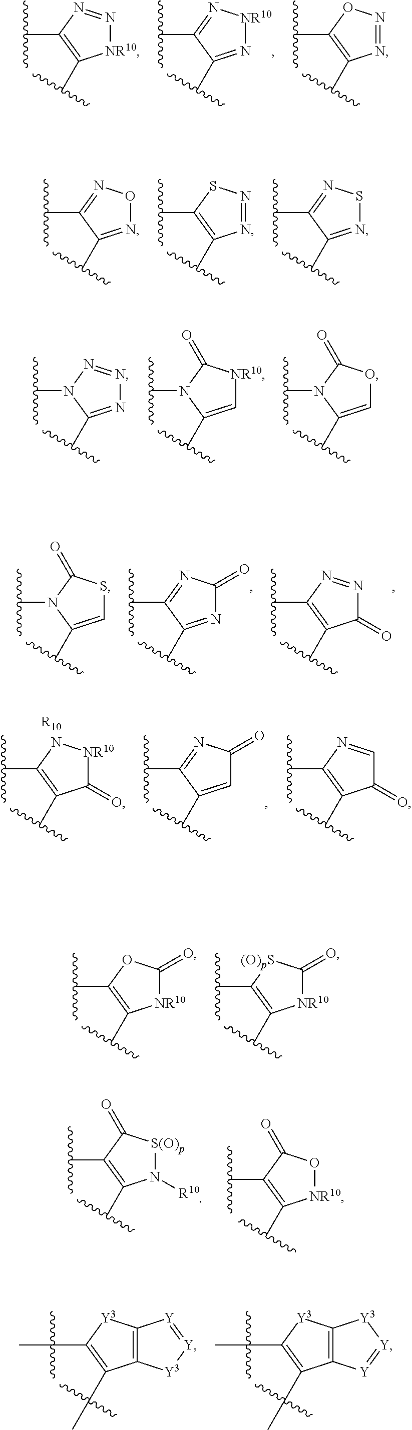

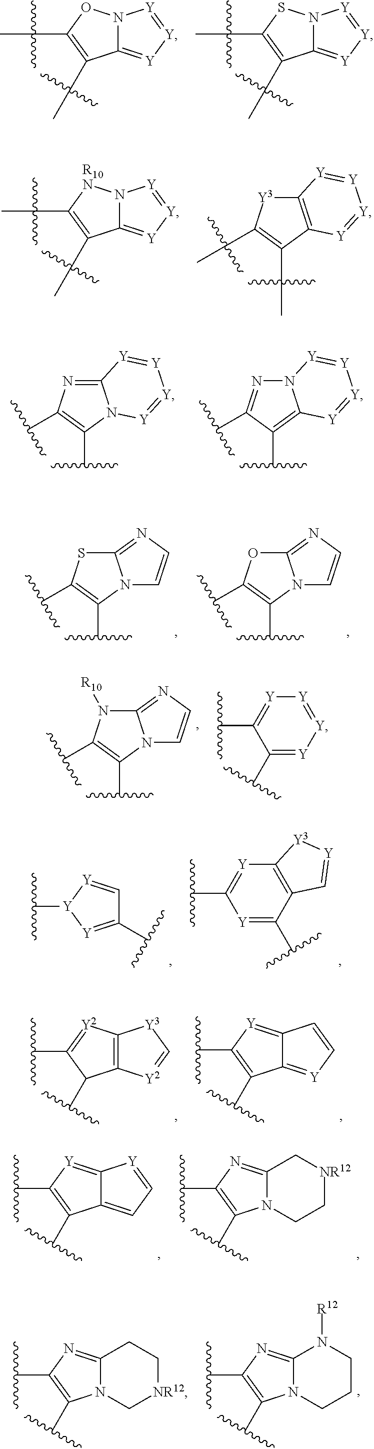

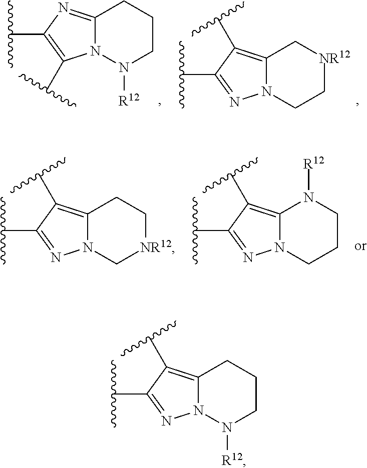

or optionally substituted variants thereof;

each Y² is independently selected from —CH═ or N; each Y³ is independently selected from C(R⁶)₂, NR⁶, O, or S; each Y⁵ is independently selected from NR⁶, O or S; and each R¹² is independently selected from hydrogen, alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (X) is also represented by Formula (Xa):

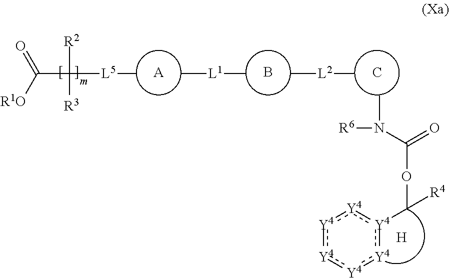

wherein A is phenyl and B is selected from

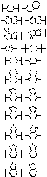

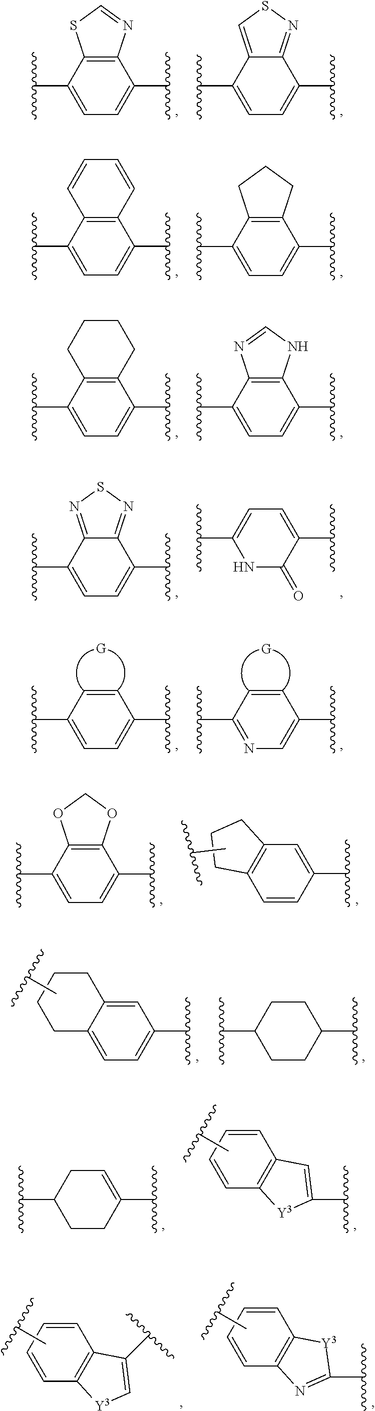

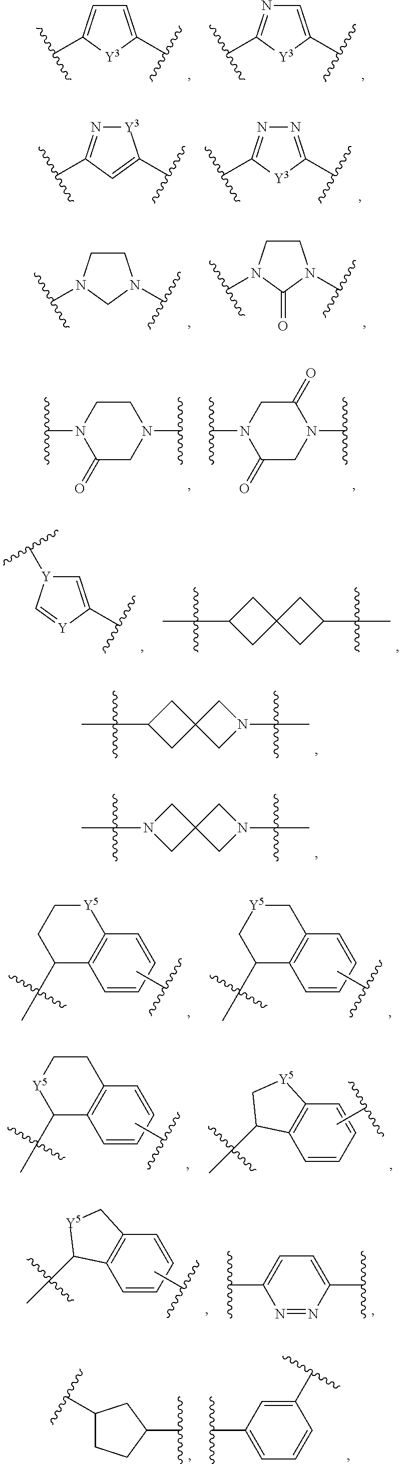

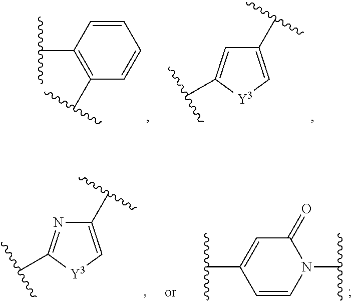

wherein the rings in A and B are unsubstituted or substituted with one or more substituents selected from alkyl, amino, haloalkyl, halogen, hydroxy, alkoxy, haloalkoxy, cyano, or oxo. In some such embodiments, both A and B are phenyl.

In some embodiments, each of A, B, and G is independently unsubstituted or substituted with one or more substituents selected from alkyl, haloalkyl, halogen, hydroxy, alkoxy, haloalkoxy, cyano, or oxo; D is selected from

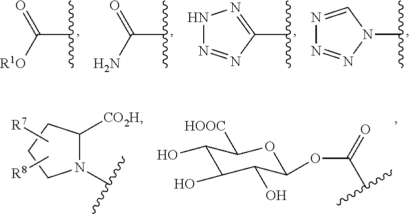

or carboxylic acid isosteres; E is absent; L⁴ is selected from

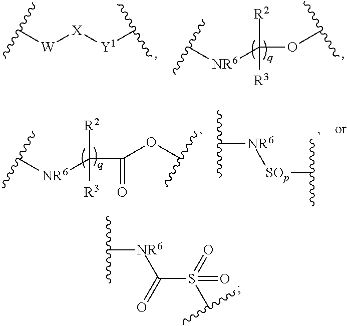

L¹ is selected from a single bond, a —O— linker, a

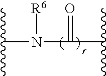

linker, a —C(O)— linker, a —CH₂— linker, a —CH₂O-linker, a —C≡C— linker, a ═C(R¹¹)— linker, or a —CH═CH— linker; L² is selected from a single bond, a —O— linker, a

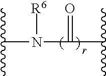

linker, a —C(O)— linker, a —CH₂— linker, a —CH₂O— linker, a —C≡C— linker, or a —CH═CH— linker; L⁵ is selected from a single bond, a —CH₂O— linker, a —CH═CH— linker, a —C≡C— linker,

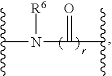

or a 4-7 membered heterocyclyl;

R¹ is selected from hydrogen or alkyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; each R⁴ and R⁵ is independently selected from hydrogen or alkyl; or R⁴ and R⁵ are joined together with the atom to which they are attached to form an optionally substituted cycloalkyl or optionally substituted heterocyclyl; each R⁶ is independently selected from hydrogen, alkyl, halogen, aryl, or C₃₋₆ cycloalkyl; each R⁷ and R⁸ is independently selected from hydrogen or C₁₋₆ alkyl; or R⁷ and R⁸ are joined together with the atom or atoms to which they are attached to form a spirocyclic heterocyclyl, a spirocyclic carbocyclyl, a fused heterocycle, or a fused carbocyclyl; each R⁹ is independently selected from hydrogen, alkyl or halogen; or two adjacent R⁹ are joined together with the atoms to which they are attached to form an optionally substituted carbocyclyl or an optionally substituted heterocyclyl; each R¹⁰ is independently selected from hydrogen, alkyl, halogen, aryl, C₃₋₆ cycloalkyl, or cyano; each R¹¹ is independently selected from hydrogen, alkyl, halogen, haloalkyl, or cyano; each R¹² is independently selected from hydrogen, alkyl, acyl, C-carboxy, C-amido, sulfinyl, sulfonyl, or S-sulfonamido; and r is an integer of 0 or 1.

In some embodiments described herein of the compounds of Formula (X) or (Xa), rings in A or B are unsubstituted.

In some embodiments described herein of the compounds of Formula (X) or (Xa), rings in A or B can be substituted. In some embodiments, rings in A or B can be substituted with one or more substituents selected from alkyl, amino, haloalkyl, halogen, hydroxy, alkoxy, haloalkoxy, cyano, or oxo. In some embodiments, rings in A or B can be substituted with one or more sulfonyl groups, for example, methanesulfonyl.

In some embodiments described herein of the compounds of Formula (X), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

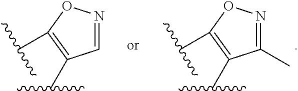

In some embodiments, C is selected from

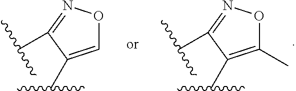

In some embodiments, C is selected from

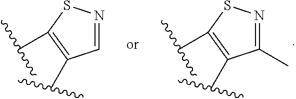

In some embodiments, C is selected from

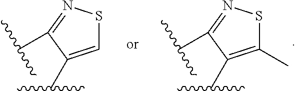

In some embodiments, C is selected from

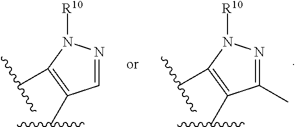

In some embodiments, C is

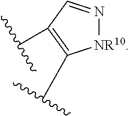

In some embodiments, C is

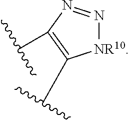

In some embodiments, C is selected from

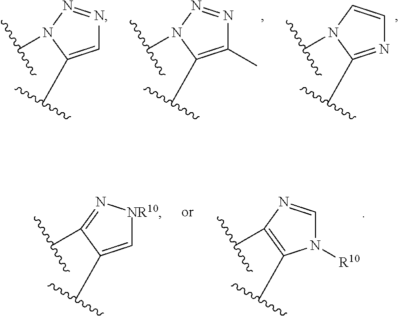

In some such embodiments, R¹⁰ is hydrogen. In some other such embodiments, R¹⁰ is C₁₋₃ alkyl or C₁₋₆ cycloalkyl.

In some embodiments, C is selected from

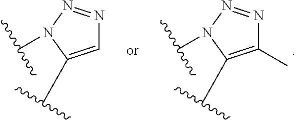

In some embodiments, C is selected from

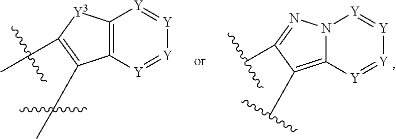

wherein Y³ is selected from O or S. In some such embodiments, Y is a CR⁶. In some other such embodiments, at least one Y is nitrogen. In some embodiments, C is optionally substituted

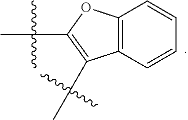 [SMILES: *c1oc2ccccc2c1*]

In some embodiments, C is selected from

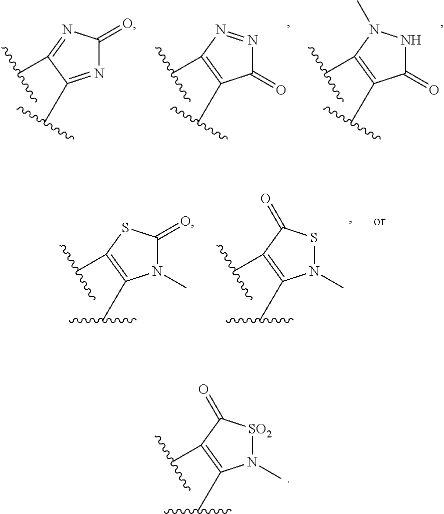

In some embodiments, m is 0. In some other embodiments, m is 1.

In some embodiments, each of R² and R³ is hydrogen. In some embodiments, at least one of R² and R³ is halogen or alkyl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L¹ is a single bond. In some embodiments, L² is a single bond.

In some embodiments, R⁶ is hydrogen. In some embodiments, R¹ is hydrogen.

In some embodiments, R⁴ is alkyl. In some other embodiments, R⁴ is hydrogen.

Some embodiments of the compound of Formula (X) or (Xa),

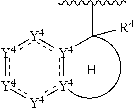

can be selected from

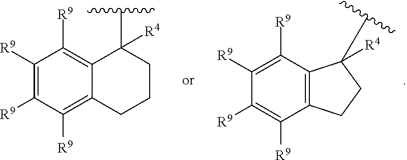

In some such embodiments, each of R⁹ is hydrogen. In some other such embodiments, at least one R⁹ is selected from C₁₋₃ alkyl or halogen.

In some embodiments,

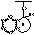

can be

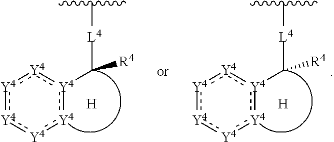

Some embodiments of the compounds of Formula (X) are selected from compounds IT057 or IT058, as shown in Table 13.

Formula (XI)

Some embodiments disclosed herein include a compound of Formula (XI) as described above or a pharmaceutically acceptable salt thereof.

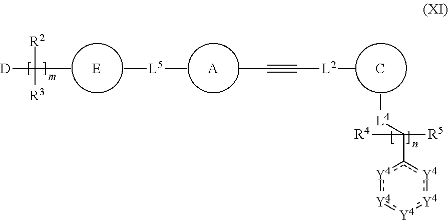

In some embodiments, C is selected from the group consisting of

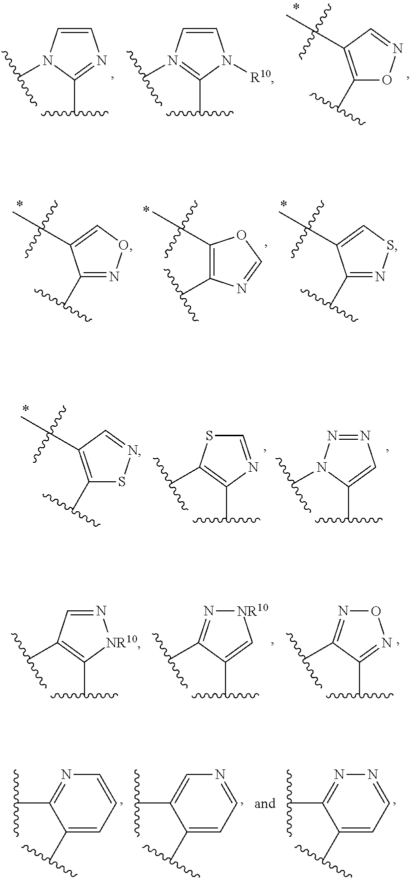

wherein C is unsubstituted or substituted with one or more substituents selected from C₁₋₃ alkyl optionally substituted with halogen or C₁₋₃ alkoxy, C₁₋₆ alkoxy, C₃₋₆ cycloalkyl, halogen, or cyano.

In some embodiments, the compound of Formula (XI) is also represented by Formula (XIa):

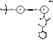

wherein A is selected from the group consisting of

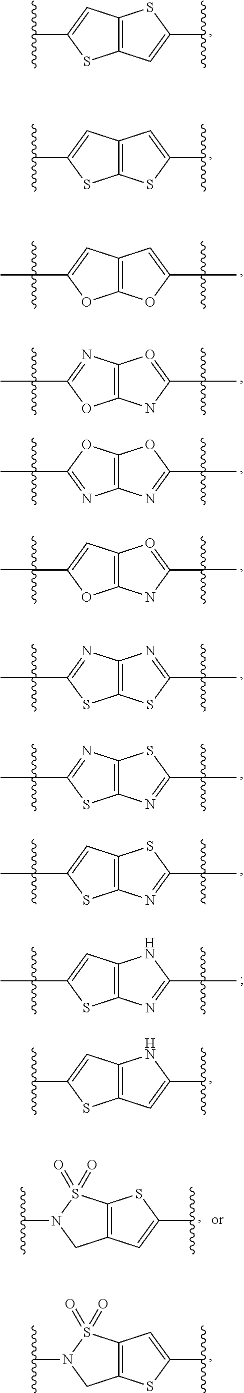

wherein the rings in A are unsubstituted or substituted with one or more substituents selected from alkyl, amino, haloalkyl, halogen, hydroxy, alkoxy, haloalkoxy, cyano, or oxo.

In some such embodiments, A is selected from

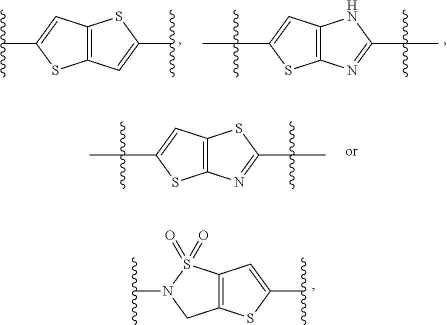 [SMILES: *c1cc2sc(*)cc2s1 ; *c1nc2sc(*)cc2[nH]1 ; *c1cc2sc(*)nc2s1 ; *c1cc2c(s1)CN(*)S2(=O)=O]

each can be optionally substituted. In one embodiment, A is optionally substituted

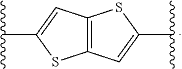 [SMILES: *c1cc2sc(*)cc2s1]

In another embodiment, A is optionally substituted

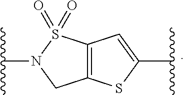 [SMILES: *c1cc2c(s1)CN(*)S2(=O)=O]

In some embodiments, A is optionally substituted with one or more substituents selected from alkyl, haloalkyl, halogen, hydroxy, alkoxy, haloalkoxy, cyano, or oxo; D is selected from —OH,

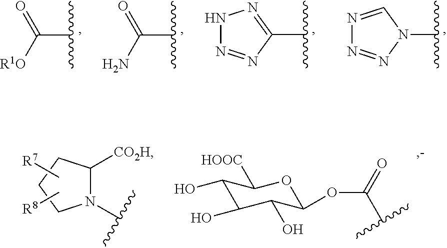

### NR¹³SOₚR¹⁴, —C(O)—NR¹³SOₚR¹⁴,

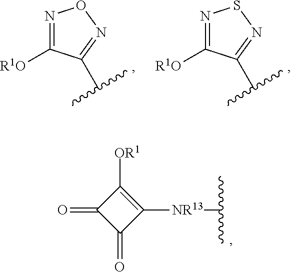

or carboxylic acid isosteres; E is absent; L² is selected from a single bond, a —O— linker, a

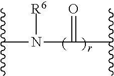

linker, a —C(O)— linker, a —CH₂— linker, a —CH₂O— linker, a —C≡C— linker, or a —CH═CH— linker; L⁵ is selected from a single bond, a —CH₂O— linker, a —CH═CH— linker, a —C≡C— linker,

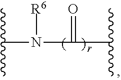

or a 4-7 membered heterocyclyl;

R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; and r is an integer of 0 or 1.

In some embodiments described herein of the compounds of Formula (XI) or (XIa), rings in A are unsubstituted.

In some embodiments described herein of the compounds of Formula (XI) or (XIa), rings in A can be substituted. In some embodiments, rings in A can be substituted with one or more substituents selected from alkyl, amino, haloalkyl, halogen, hydroxy, alkoxy, haloalkoxy, cyano, or oxo. In some embodiments, rings in A can be substituted with one or more sulfonyl groups, for example, methanesulfonyl.

In some embodiments described herein of the compounds of Formula (XI), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E is unsubstituted or substituted. In some embodiments, E is substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other such embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is hydrogen. In some other embodiments, R⁶ is C₁₋₃ alkyl.

In some embodiments, R⁴ is alkyl optionally substituted with halogen. In some other embodiments, R⁴ is hydrogen.

In some embodiments of the compound of Formula (XI) or (XIa),

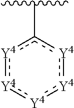

an be

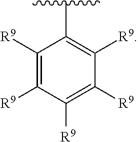

In some embodiments, each of R⁹ is hydrogen. In some other embodiments, at least one R⁹ is selected from C₁₋₃ alkyl or halogen.

In some embodiments,

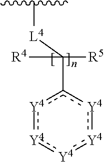

can be

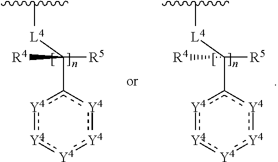

Some embodiments of the compounds of Formula (XI) are selected from compounds of Table 10A as shown below, and pharmaceutically acceptable salts thereof.

Some embodiments of the compounds Formula (XI) are selected from IT101 IT106, IT108, IT115, or IT116 as shown in Table 13.

Formula (XII)

Some embodiments disclosed herein include a compound of Formula (XII) as described above or a pharmaceutically acceptable salt thereof.

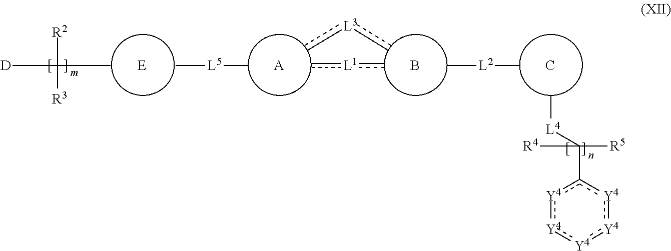

In some embodiments, each of A and B can be an acetylene or selected from the group consisting of

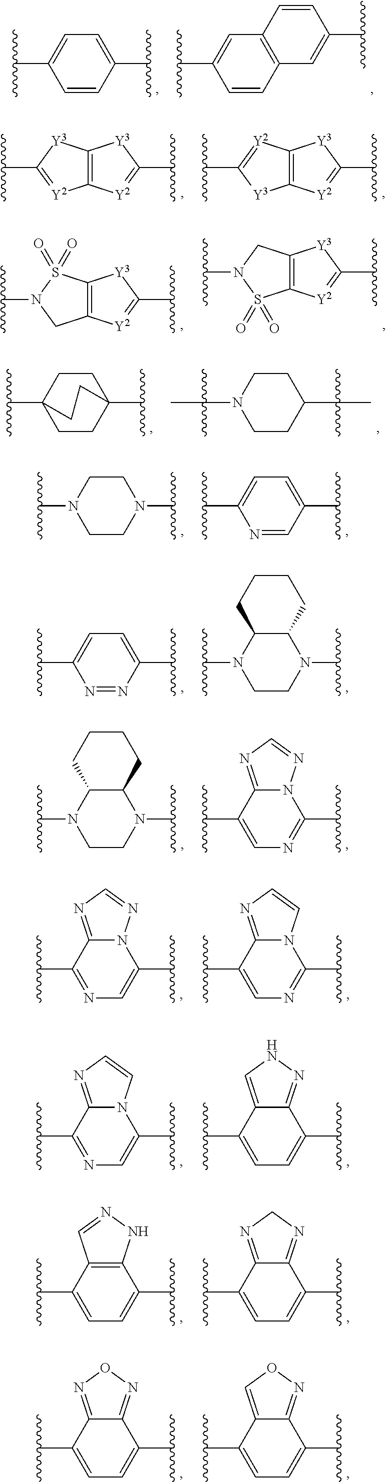

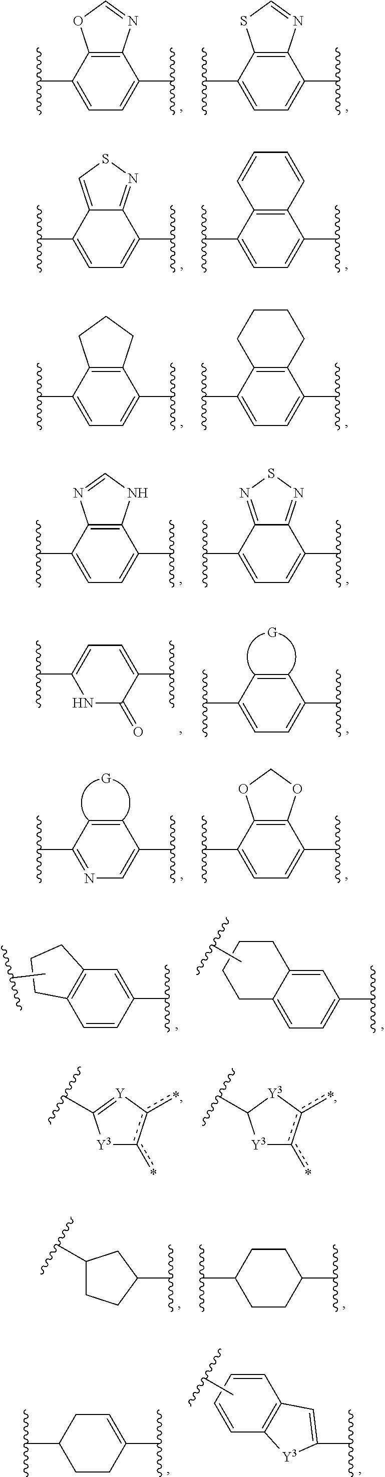

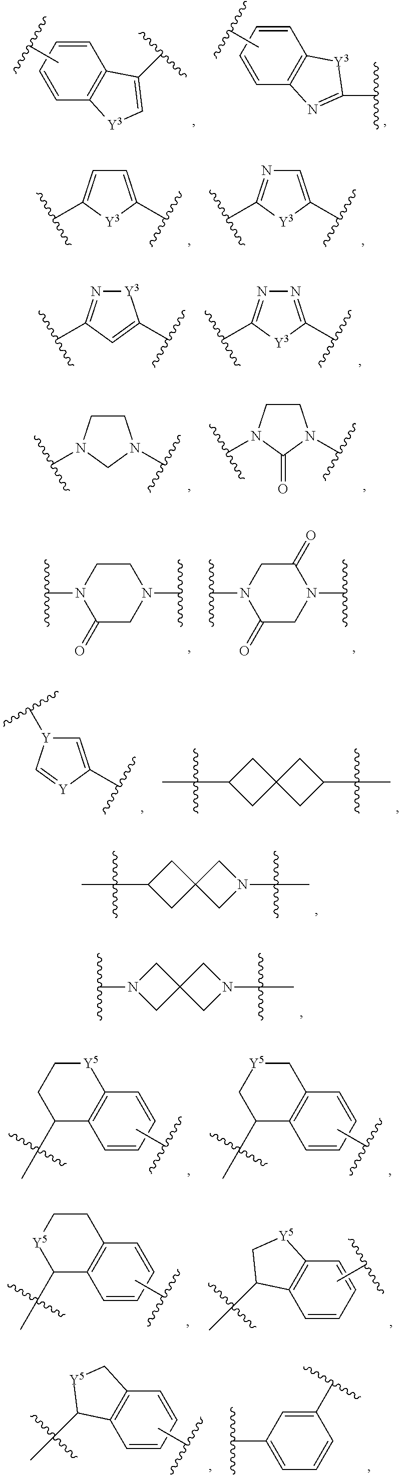

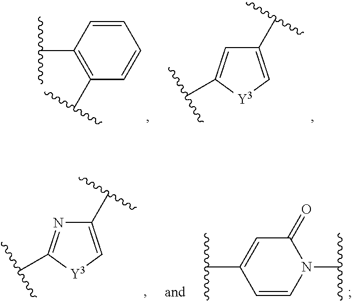

wherein each * is a point of attachment of A or B to L¹ or L³, and wherein the rings in A are optionally substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is optionally with one or more substituents selected from alkyl, amino, haloalkyl, halogen, or oxo;

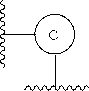

is selected from

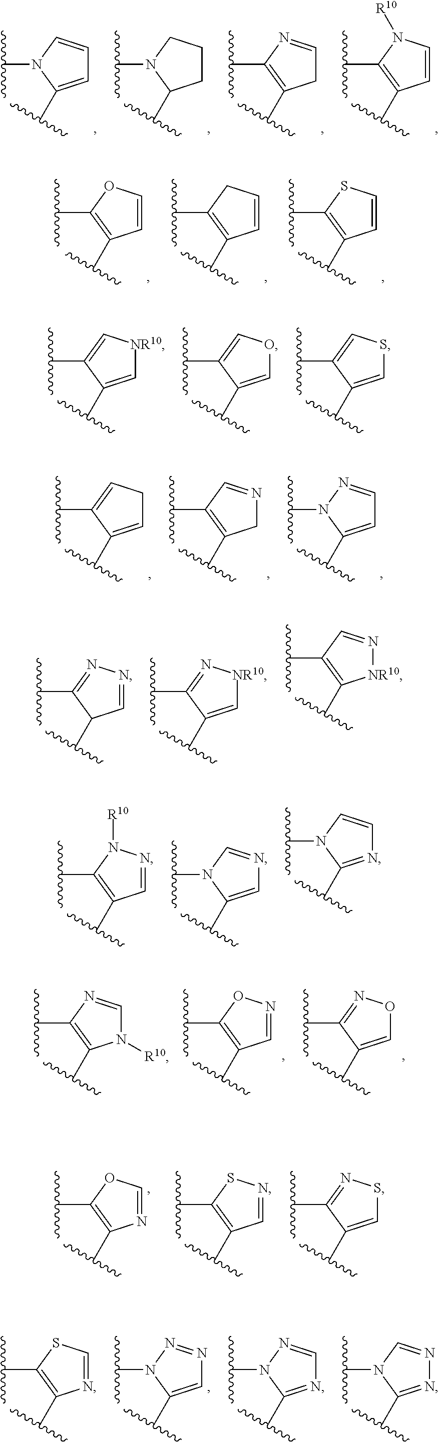

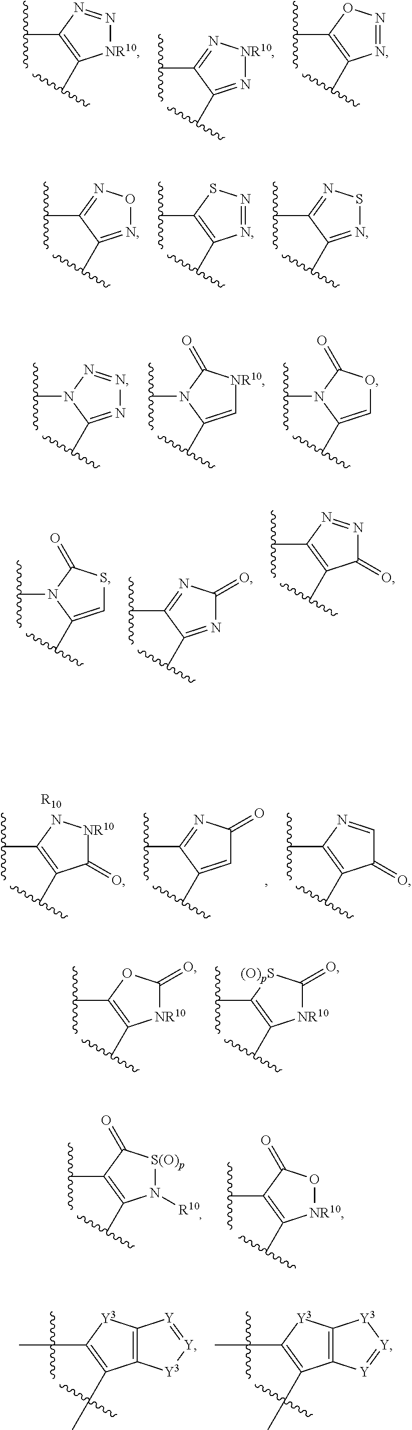

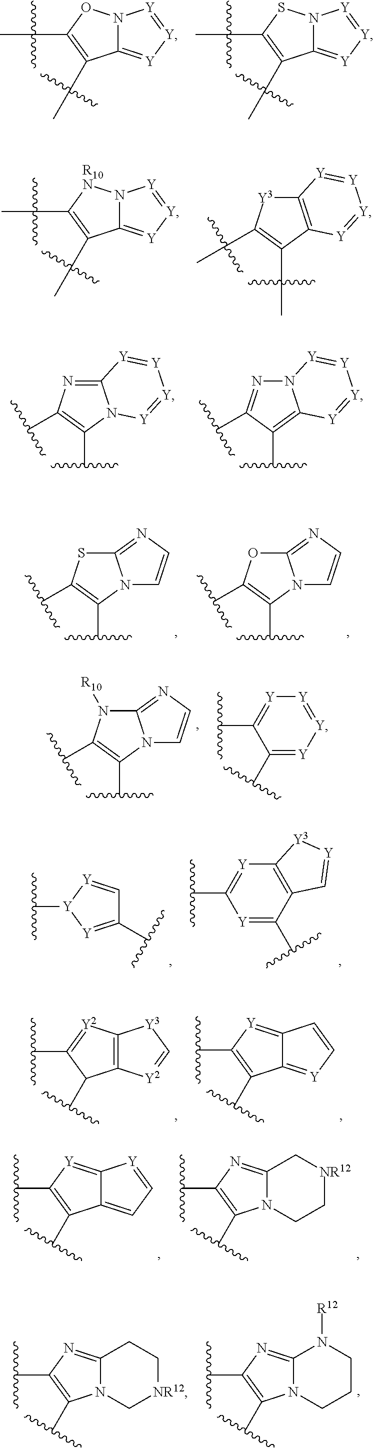

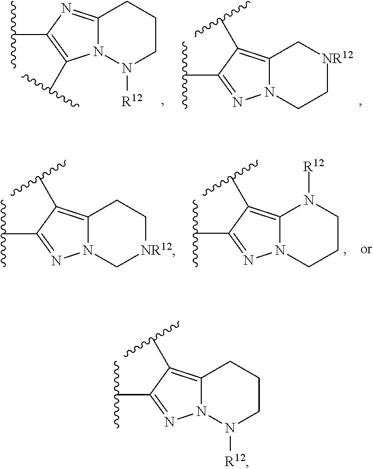

or optionally substituted variants thereof;

each Y² is independently selected from —CH═ or N; each Y³ is independently selected from C(R⁶)₂, NR⁶, O or S; each Y⁵ is independently selected from NR⁶, O or S;

each C₃₋₇ cycloalkyl, C₃₋₇ heterocyclyl, and 5-10 membered heteroaryl of R² or R³ is optionally substituted with one or more substituents selected from alkyl, haloalkyl, halogen, hydroxy, alkoxy, haloalkoxy, cyano, or oxo; and

each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (XII) is also represented by Formula (XIIa):

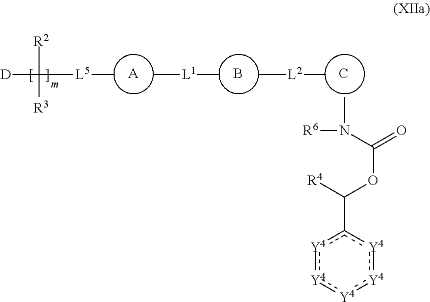

wherein each A or B can be selected from acetylene,

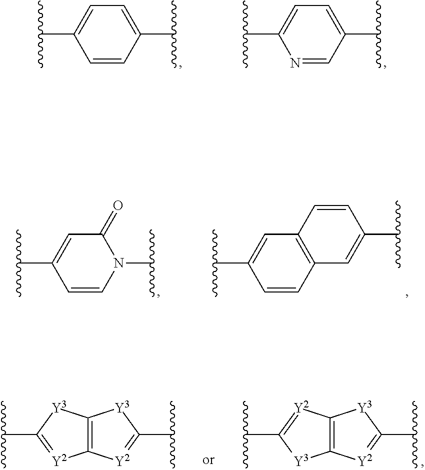

wherein the rings in A or B are unsubstituted or substituted with one or more substituents selected from alkyl, amino, haloalkyl, halogen, hydroxy, alkoxy, haloalkoxy, cyano, sulfonyl or oxo.

In some embodiments, wherein the ring system in each A and B is independently optionally substituted with one or more substituents selected from the group consisting of alkyl, amino, haloalkyl, halogen, hydroxy, alkoxy, haloalkoxy, cyano, or oxo; E is absent or optionally substituted with one or more substituents selected from the group consisting of alkyl, amino, haloalkyl, halogen, hydroxy, alkoxy, haloalkoxy, cyano or oxo.

In some embodiments, both A and B are

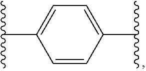 [SMILES: *c1ccc(*)cc1]

each unsubstituted or substituted with one or more substituents selected from alkyl, amino, haloalkyl, halogen, hydroxy, alkoxy, haloalkoxy, cyano, or oxo.

In some embodiments, one of A or B is

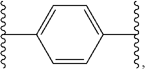 [SMILES: *c1ccc(*)cc1]

and the other A or B is selected from

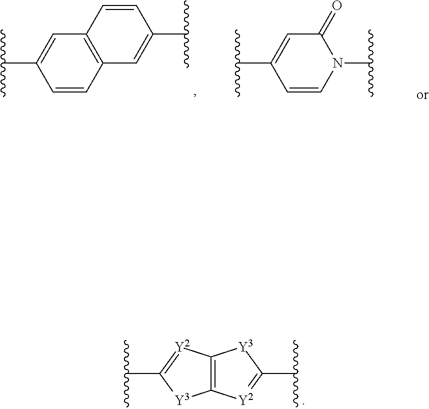

In some such embodiments, A is

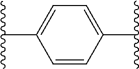 [SMILES: *c1ccc(*)cc1]

and B is selected from

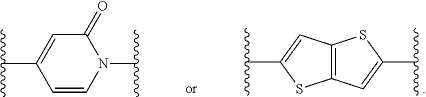 [SMILES: *c1ccn(*)c(=O)c1 ; *c1cc2sc(*)cc2s1]

In some such embodiments, B is

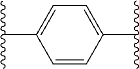 [SMILES: *c1ccc(*)cc1]

and A is selected from

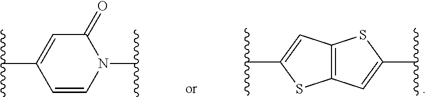 [SMILES: *c1ccn(*)c(=O)c1 ; *c1cc2sc(*)cc2s1]

In some of these embodiments, each A or B can be optionally substituted.

In some embodiments, one of A or B is acetylene and the other A or B is selected from

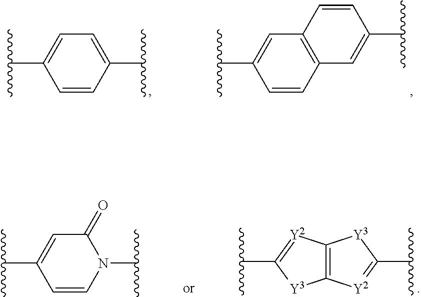

In some of these embodiments, each A or B can be optionally substituted.

In some embodiments described herein of the compounds of Formula (XII) or (XIIa), rings in A or B are unsubstituted.

In some embodiments described herein of the compounds of Formula (XII) or (XIIa), rings in A or B are substituted. In some embodiments, rings in A or B can be substituted with one or more substituents selected from alkyl, amino, haloalkyl, halogen, hydroxy, alkoxy, haloalkoxy, cyano, or oxo. In some embodiments, rings in A or B can be substituted with one or more sulfonyl groups, for example, methanesulfonyl.

In some embodiments described herein of the compounds of Formula (XII), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

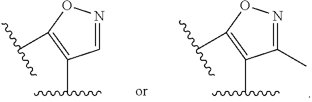

In some embodiments, C is selected from

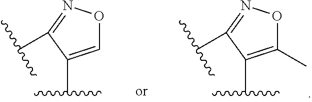

In some embodiments, C is selected from

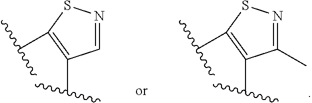

In some embodiments, C is selected from

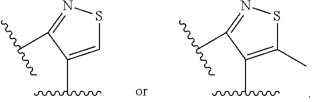

In some embodiments, C is selected from

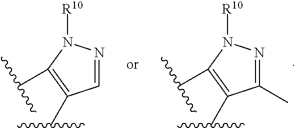

In some embodiments, C is

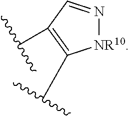

In some embodiments, C is

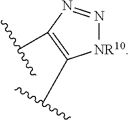

In some embodiments, C is

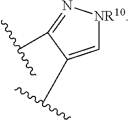

In some embodiments, C is selected from

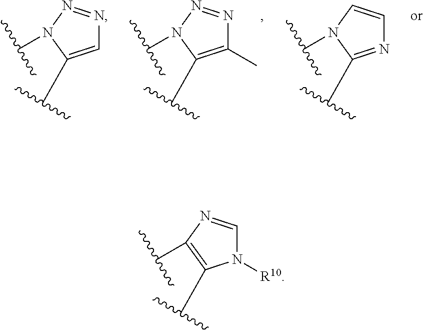

In some such embodiments, R¹⁰ is hydrogen. In some other such embodiments, R¹⁰ is C₁₋₃ alkyl or C₃₋₆ cycloalkyl.

In some embodiments, C is selected from

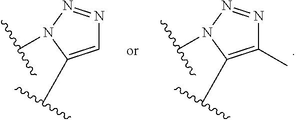

In some embodiments, C is selected from

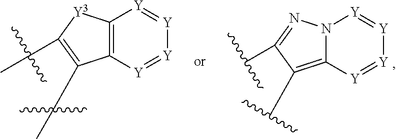

wherein Y³ is selected from O or S. In some such embodiments, Y is a CR⁶. In some other such embodiments, at least one Y is nitrogen. In some embodiments, C is optionally substituted

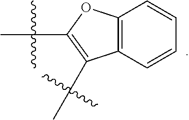 [SMILES: *c1oc2ccccc2c1*]

In some embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other such embodiments, R¹ is optionally substituted aryl.

In some embodiments, m is 1. In some other embodiments, m is 2.

In some embodiments, at least one of R² and R³ is halogen. In some other embodiments, at least one of R² and R³ is haloalkyl.

In some embodiments, R² is hydrogen and R³ is selected from optionally substituted C₃₋₆ cycloalkyl. In some such embodiments, R³ is optionally substituted cyclobutyl.

In some embodiments, R² is hydrogen and R³ is selected from optionally substituted 3-6 membered heterocyclyl. In some such embodiments, R³ is optionally substituted oxetane.

In some embodiments, R² is hydrogen and R³ is selected from optionally substituted 5-10 membered heteroaryl. In some such embodiments, R³ is selected from thiazolyl or oxazolyl, each can be optionally substituted.

In some other embodiments, R² and R³ are joined together with the atom to which they are attached to form a C₃₋₆ cycloalkyl substituted by one or more halogen.

In some embodiments, L⁵ is a single bond. In some other embodiments, L⁵ is a —O-linker.

In some embodiments, L² is a single bond.

In some embodiments, L¹ is a single bond.

In some embodiments, R⁶ is hydrogen. In some other embodiments, R⁶ is C₁₋₃ alkyl.

In some embodiments, R⁴ is alkyl optionally substituted with halogen. In some other embodiments, R⁴ is hydrogen.

In some embodiments of the compound of Formula (XII) or (XIIa),

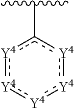

can be

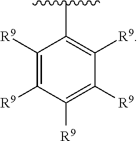

In some embodiments, each of R⁹ is hydrogen. In some other embodiments, at least one R⁹ is selected from C₁₋₃ alkyl or halogen.

In some embodiments,

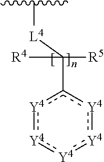

can be

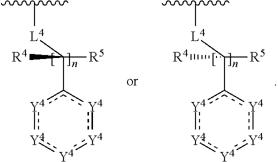

Some embodiments of the compounds of Formula (XII) are selected from compounds of Table 12A and Table 12B, and pharmaceutically acceptable salts thereof.

Some embodiments of the compounds of Formula (XII) are selected from IT123, IT136, IT150, IT151, IT172 or IT228 as shown in Table 13.

Formula (XIII)

Some embodiments disclosed herein include a compound of Formula (XIII) as described above or a pharmaceutically acceptable salt thereof.

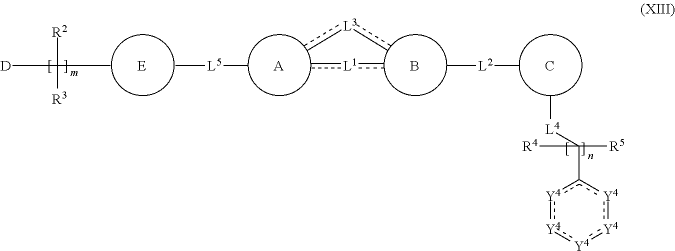

In some embodiments, each of A and B can be an acetylene or selected from the group consisting of

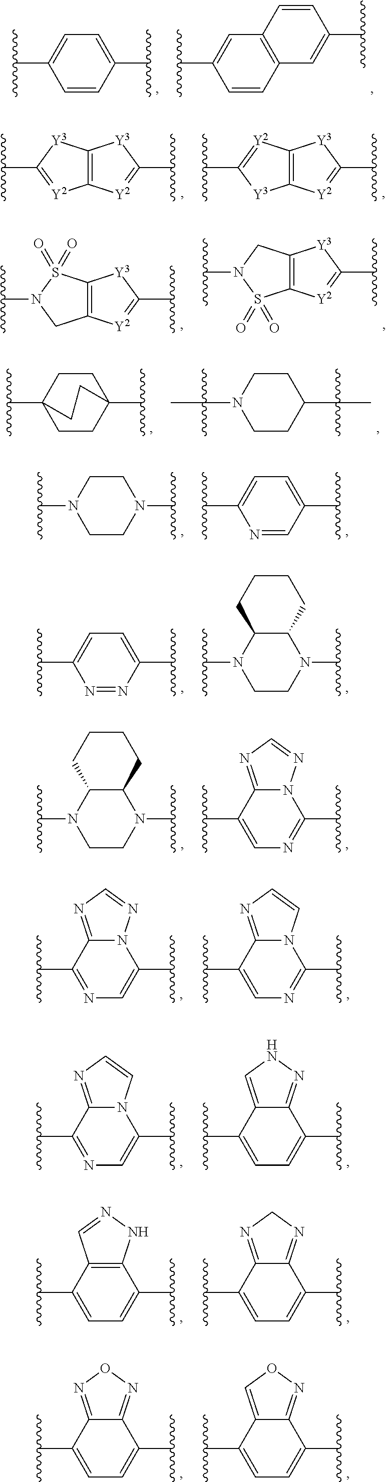

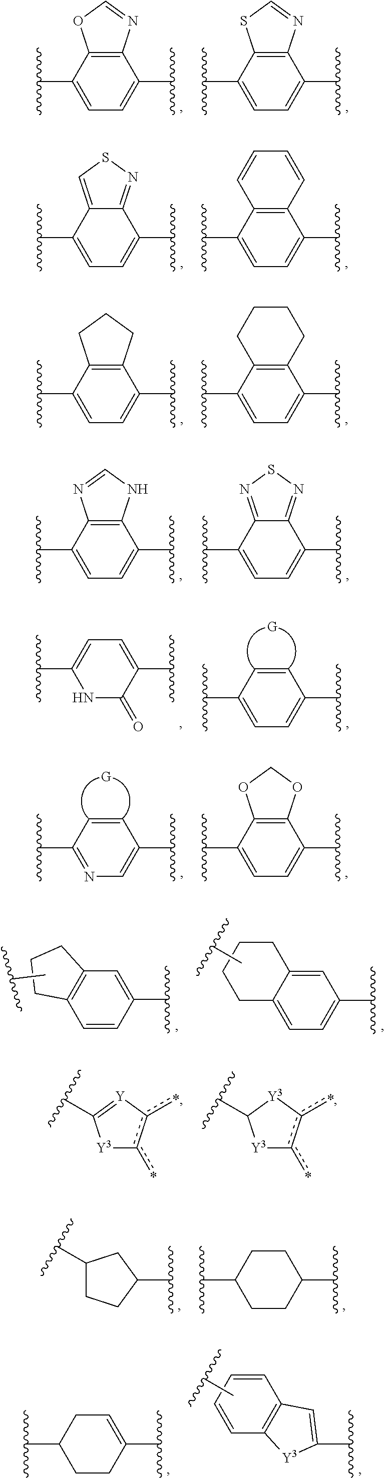

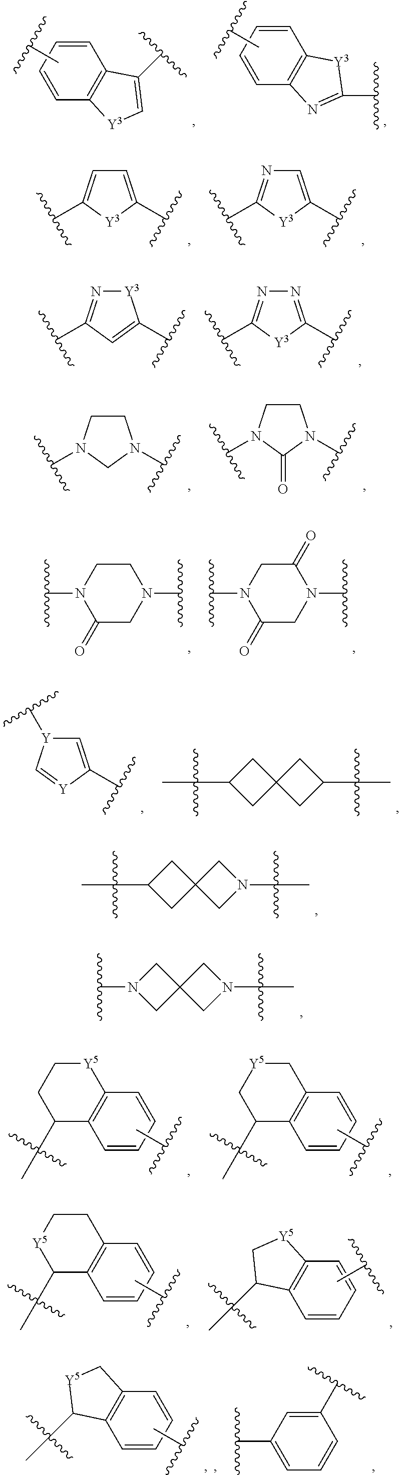

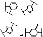

wherein each * is a point of attachment of A or B to L¹ or L³, and wherein the rings in A or B are optionally substituted with one or more substituents selected from alkyl, amino, haloalkyl, halogen, hydroxy, alkoxy, haloalkoxy, cyano, sulfonyl or oxo;

E is absent or optionally substituted with optionally substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is optionally with one or more substituents selected from alkyl, amino, haloalkyl, halogen, or oxo;

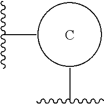

is selected from

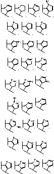

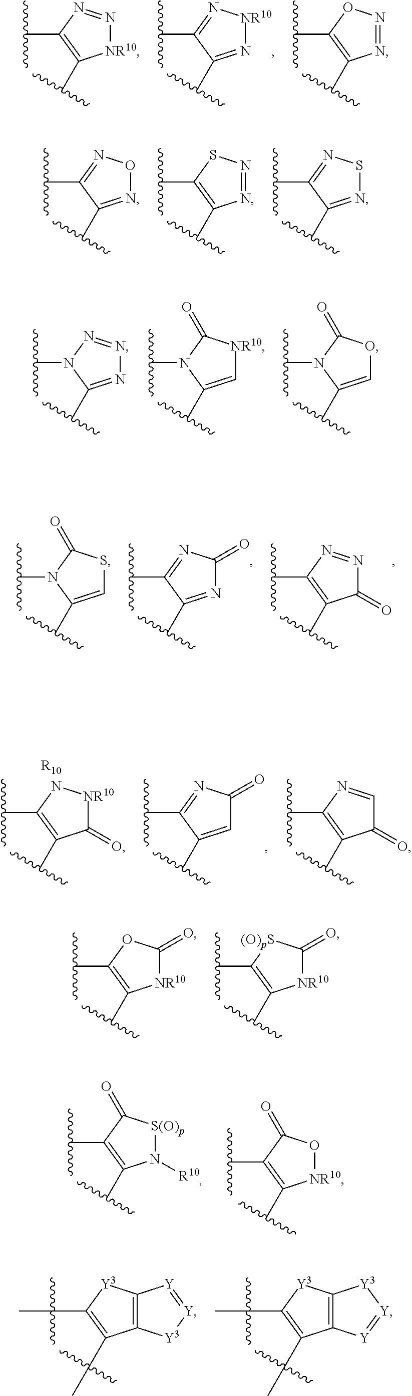

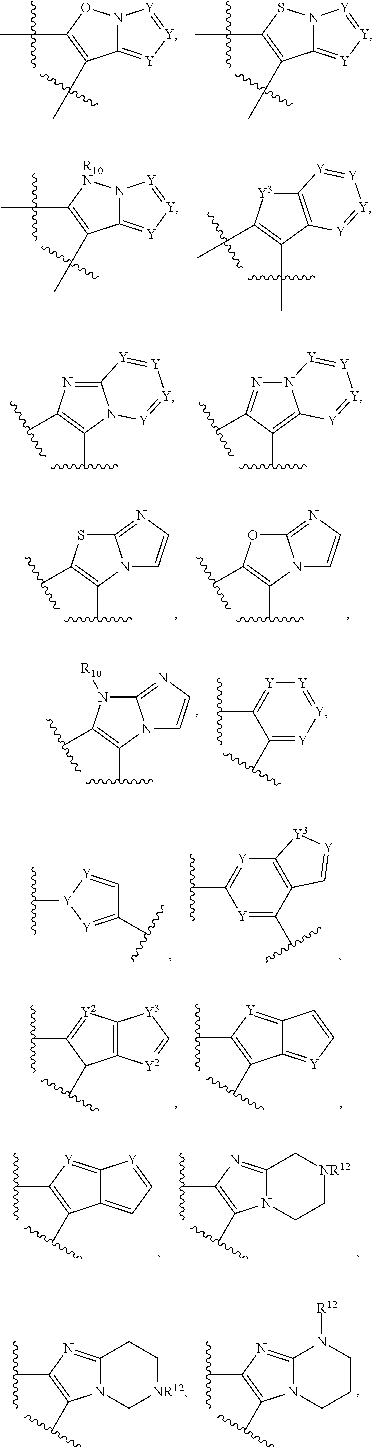

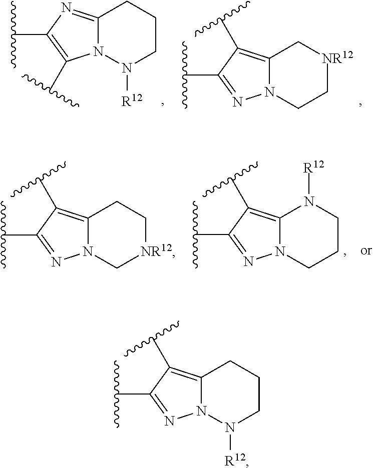

or optionally substituted variants thereof;

each Y² is independently selected from —CH═ or N; each Y³ is independently selected from C(R⁶)₂, NR⁶, O or S; each Y⁵ is independently selected from NR⁶, O or S; each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (XIII) is also represented by Formula (XIIIa):

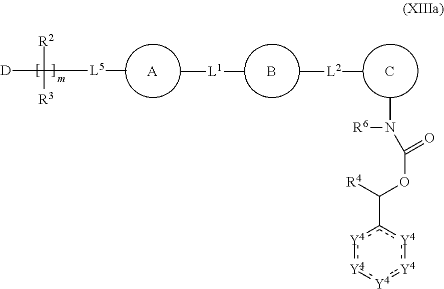

wherein each A or B can be selected from acetylene,

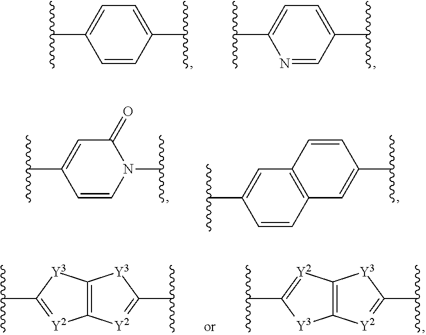

wherein the rings in A or B are unsubstituted or substituted with one or more substituents selected from alkyl, amino, haloalkyl, halogen, hydroxy, alkoxy, haloalkoxy, cyano, sulfonyl or oxo.

In some embodiments, the ring system in each A and B is independently optionally substituted with one or more substituents selected from the group consisting of alkyl, amino, haloalkyl, halogen, hydroxy, alkoxy, haloalkoxy, cyano, or oxo; E is absent or optionally substituted with one or more substituents selected from the group consisting of alkyl, amino, haloalkyl, halogen, hydroxy, alkoxy, haloalkoxy, cyano or oxo.

In some embodiments, D is —SO₂R¹⁵. In some such embodiments, R¹⁵ is selected from hydrogen, C₁₋₆ alkyl, C₃₋₆ cycloalkyl, or —CH₂—C₃₋₆cycloalkyl.

In some embodiments, D is —SO₂NR¹⁶R¹⁷. In some such embodiments, each of R¹⁶ and R¹⁷ is selected from hydrogen, C₁₋₆ alkyl, or acyl. In some other such embodiments, le and R¹⁷ are joined together with the atom to which they are attached to form an optionally substituted C₃₋₆ cycloalkyl.

In some embodiments, both A and B are

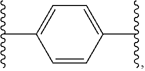 [SMILES: *c1ccc(*)cc1]

each can be optionally substituted.

In some embodiments, one of A or B is

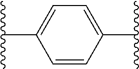 [SMILES: *c1ccc(*)cc1]

and the other A or B is

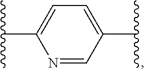 [SMILES: *c1ccc(*)nc1]

each can be optionally substituted.

In some embodiments described herein of the compounds of Formula (XIII) or (XIIIa), rings in A or B are unsubstituted.

In some embodiments described herein of the compounds of Formula (XIII) or (XIIIa), rings in A or B can be substituted. In some embodiments, rings in A or B can be substituted with one or more substituents selected from alkyl, amino, haloalkyl, halogen, hydroxy, alkoxy, haloalkoxy, cyano, or oxo. In some embodiments, rings in A or B can be substituted with one or more sulfonyl groups, for example, methanesulfonyl.

In some embodiments described herein of the compounds of Formula (XIII), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

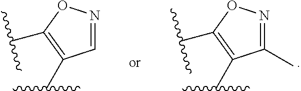

In some embodiments, C is selected from

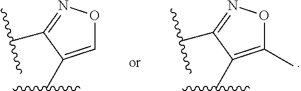

In some embodiments, C is selected from

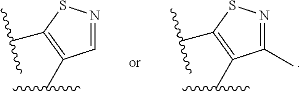

In some embodiments, C is selected from

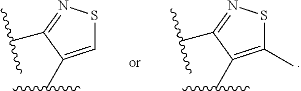

In some embodiments, C is selected from

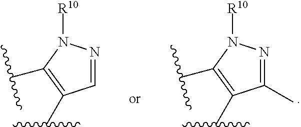

In some embodiments, C is

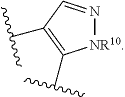

In some embodiments, C is

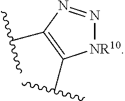

In some embodiments, C is selected from

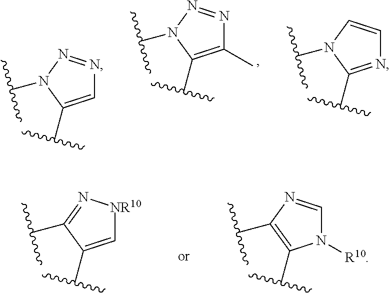

In some such embodiments, R¹⁰ is hydrogen. In some other such embodiments, R¹⁰ is C₁₋₃ alkyl or C₃₋₆ cycloalkyl.

In some embodiments, C is selected from

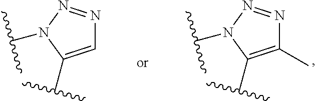

In some embodiments, C is selected from

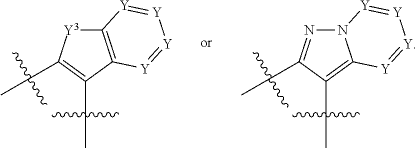

wherein Y³ is selected from O or S. In some such embodiments, Y is a CR⁶. In some other such embodiments, at least one Y is nitrogen. In some embodiments, C is optionally substituted

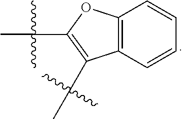 [SMILES: *c1oc2ccccc2c1*]

In some embodiments, m is 1. In some other embodiments, m is 0.

In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond. In some embodiments, L¹ is a single bond.

In some embodiments, R⁶ is hydrogen. In some other embodiments, R⁶ is C₁₋₃ alkyl.

In some embodiments, R⁴ is alkyl optionally substituted with halogen. In some other embodiments, R⁴ is hydrogen.

In some embodiments of the compound of Formula (XIII) or (XIIIa),

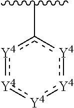

can be

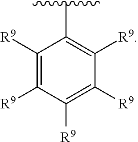

In some embodiments, each of R⁹ is hydrogen. In some other embodiments, at least one R⁹ is selected from C₁₋₃ alkyl or halogen.

In some embodiments,

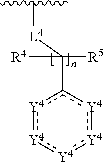

can be

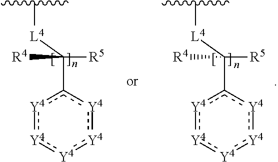

Some embodiments of the compounds of Formula Formula (XIII) are selected from IT124, IT128-IT131, IT138, IT139, IT153, IT173, IT174, or IT228 as shown in Table 13.

Formula (XIV)

Some embodiments disclosed herein include a compound of Formula (XIV) as described above or a pharmaceutically acceptable salt thereof.

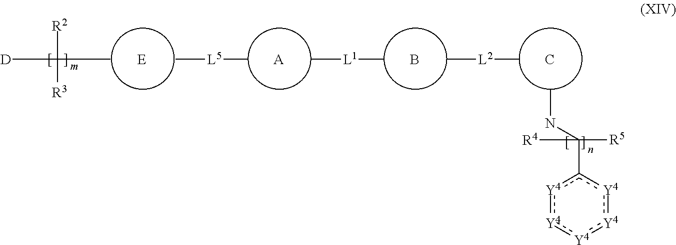

In some embodiments, each of A and B can be an acetylene or selected from the group consisting of

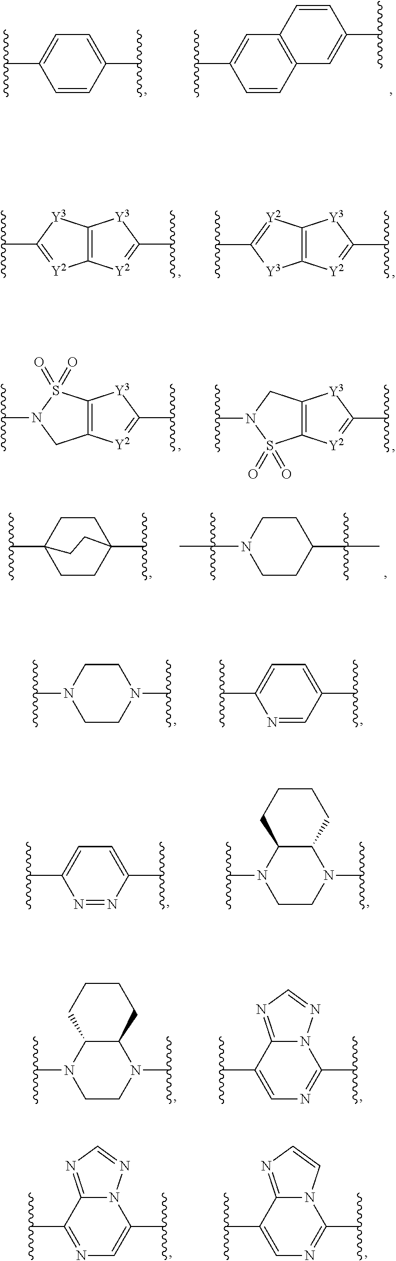

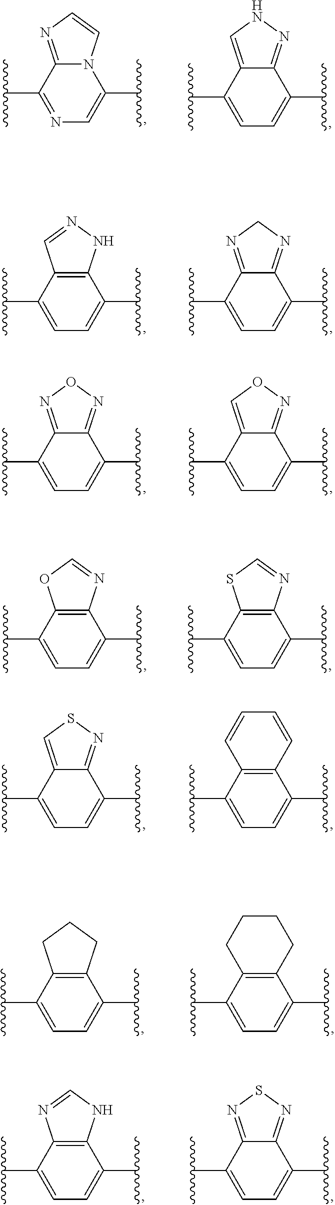

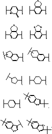

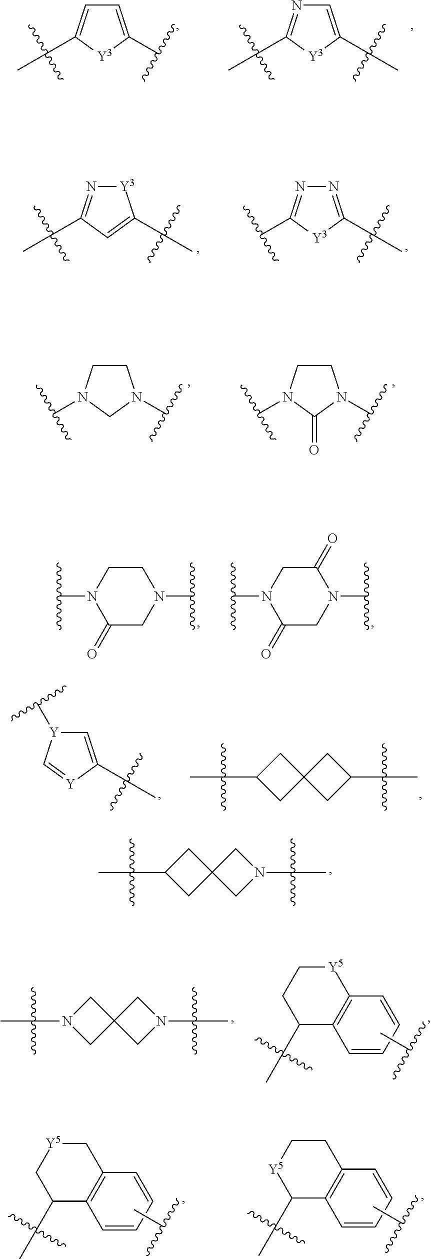

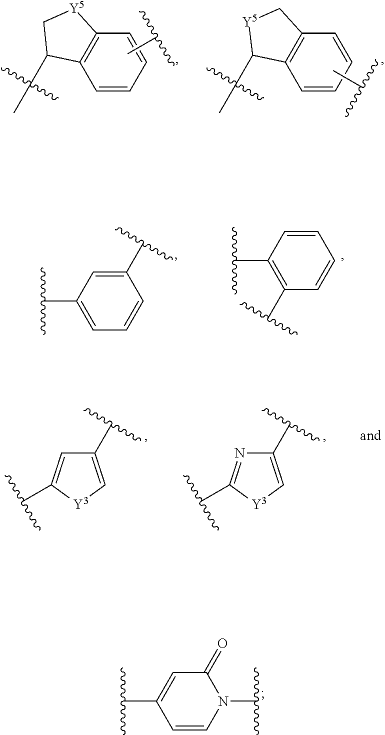

wherein the rings in A or B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, or oxo;

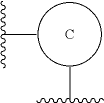

is selected from

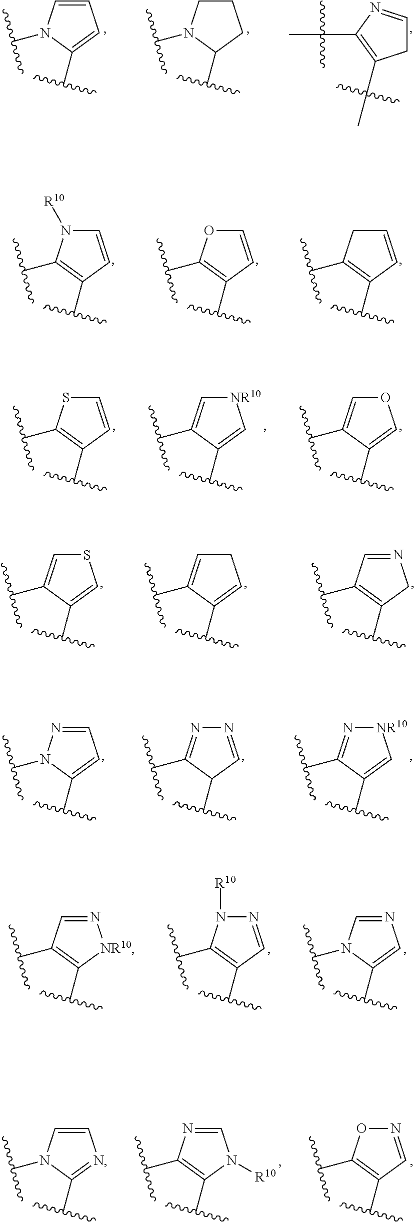

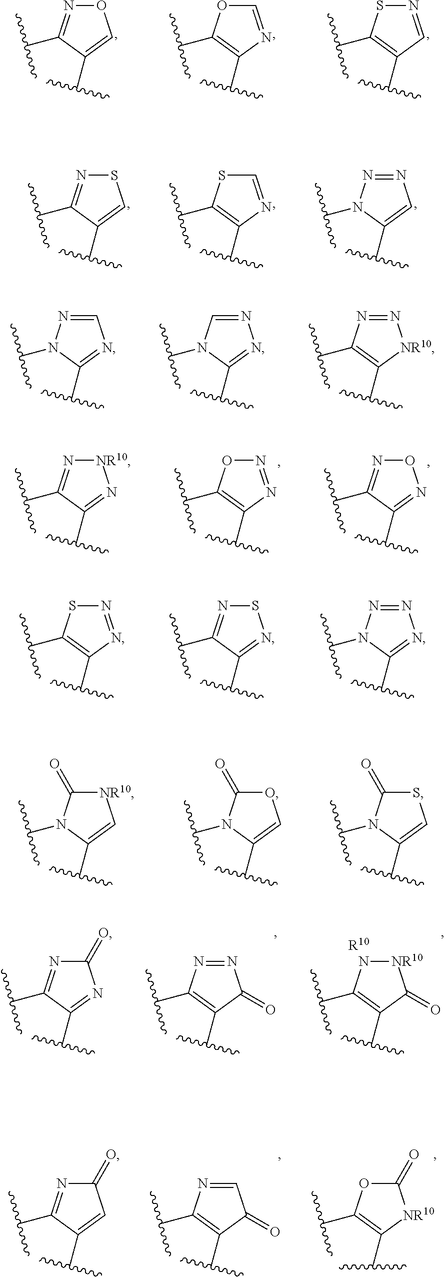

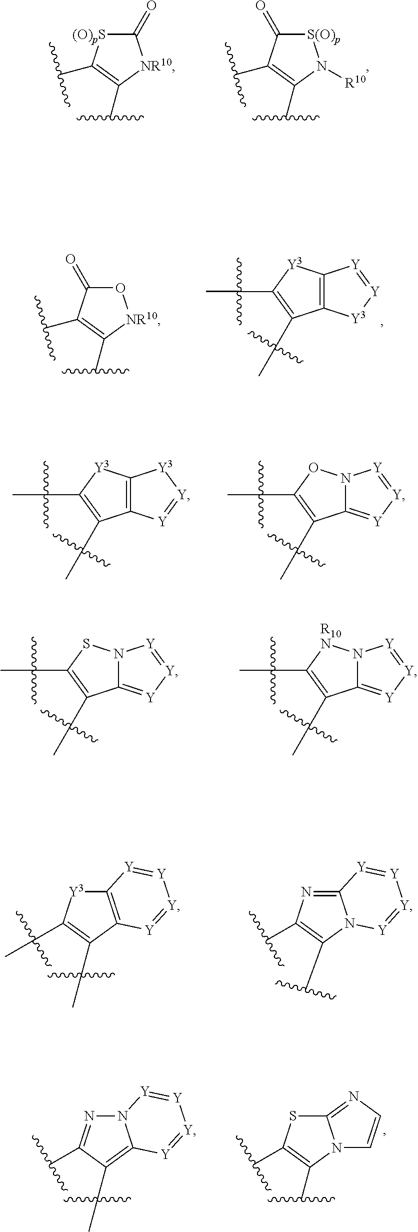

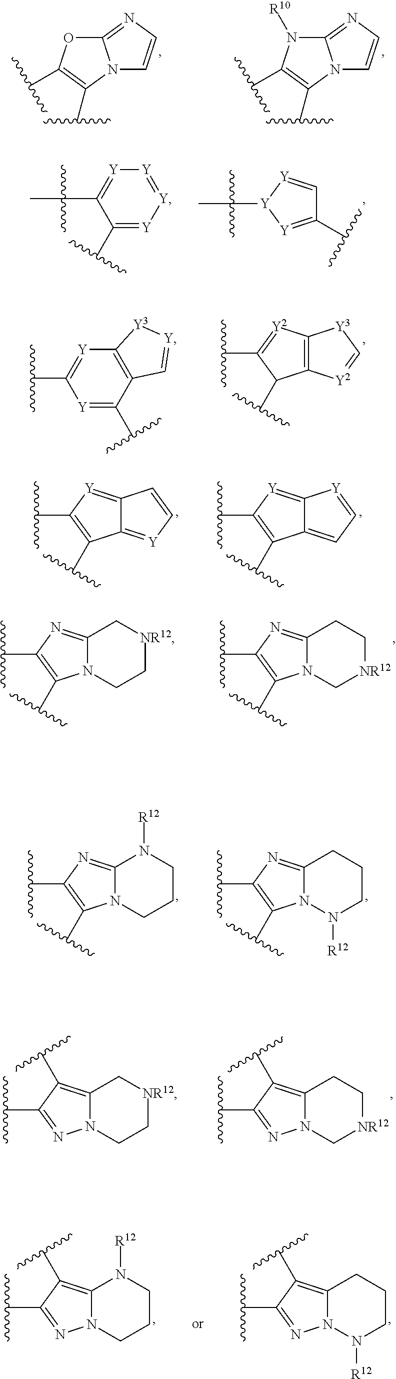

or optionally substituted variants thereof;

each Y² is independently selected from —CH═ or N; each Y³ is independently selected from C(R⁶)₂, NR⁶, O or S; each Y⁵ is independently selected from NR⁶, O or S; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, C ring cannot be isoxazole or alkyl substituted isoxazole.

In some embodiments, the compound of Formula (XIV) is also represented by Formula (XIVa):

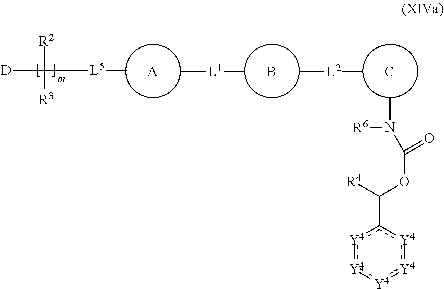

wherein each of A and B can be selected from

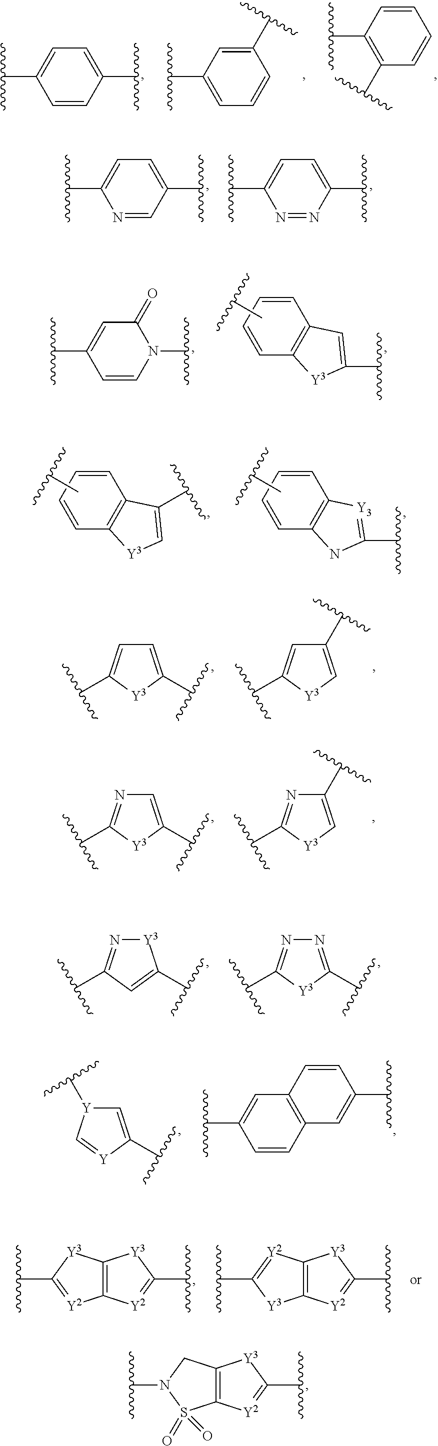

wherein the rings in A and B are unsubstituted or substituted with one or more substituents selected from alkyl, amino, haloalkyl, halogen, hydroxy, alkoxy, haloalkoxy, cyano, sulfonyl or oxo.

In some embodiments, the ring system in each A and B is independently optionally substituted with one or more substituents selected from the group consisting of alkyl, amino, haloalkyl, halogen, hydroxy, alkoxy, haloalkoxy, cyano, or oxo; E is absent or optionally substituted with one or more substituents selected from the group consisting of alkyl, amino, haloalkyl, halogen, hydroxy, alkoxy, haloalkoxy, cyano or oxo.

In some embodiments, both s and u in L¹ are 0. In some embodiments, at least one of s and u in L¹ is 0. In some embodiments, one of s or u is 0 and the other s or u is 1 in L¹.

In some embodiments, L¹ is

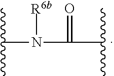

In some embodiments, L¹ is

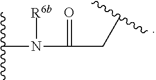

In some embodiments, L¹ is

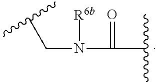

In some such embodiments, R^{6b} is hydrogen. In some other such embodiments, R^{6b} is C₁₋₃ alkyl.

In some embodiments, A is

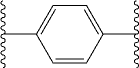 [SMILES: *c1ccc(*)cc1]

and B is selected from

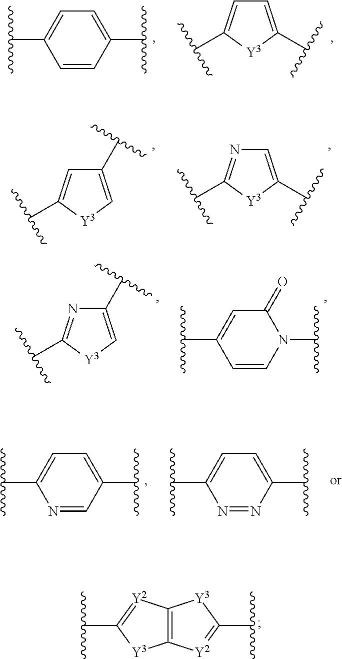

and wherein each of the rings in A or B can be optionally substituted. In some such embodiments, B is optionally substituted

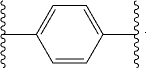 [SMILES: *c1ccc(*)cc1]

In some such embodiments, B is optionally substituted

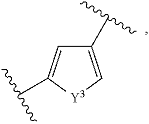

wherein Y³ is O or S. In some such embodiments, B is optionally substituted

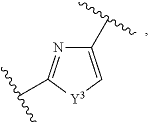

wherein Y³ is O or S.

In someembodiments, B is

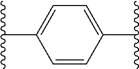 [SMILES: *c1ccc(*)cc1]

and A is selected from

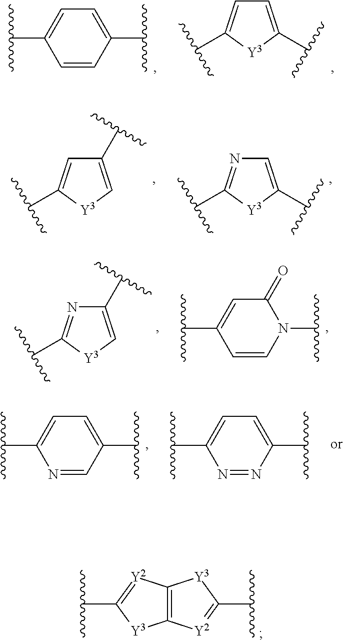

and wherein each of the rings in A or B can be optionally substituted. In some such embodiments, A is optionally substituted

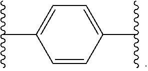 [SMILES: *c1ccc(*)cc1]

In some such embodiments, A is optionally substituted

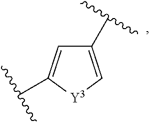

wherein Y³ is O or S. In some such embodiments, A is optionally substituted

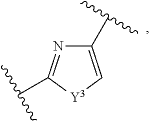

wherein Y³ is O or S.

In some embodiments described herein of the compounds of Formula (XIV) or (XIVa), rings in A or B are unsubstituted.

In some embodiments described herein of the compounds of Formula (XIV) or (XIVa), rings in A or B can be substituted. In some embodiments, rings in A or B can be substituted with one or more substituents selected from alkyl, amino, haloalkyl, halogen, hydroxy, alkoxy, haloalkoxy, cyano, or oxo. In some embodiments, rings in A or B can be substituted with one or more sulfonyl groups, for example, methanesulfonyl.

In some embodiments described herein of the compounds of Formula (XIV), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

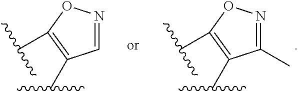

In some embodiments, C is selected from

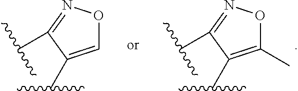

In some embodiments, C is selected from

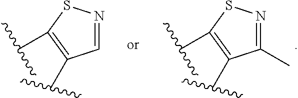

In some embodiments, C is selected from

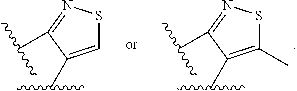

In some embodiments, C is selected from

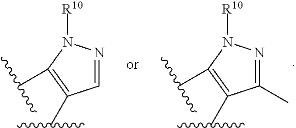

In some embodiments, C is

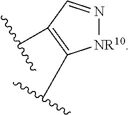

In some embodiments, C is

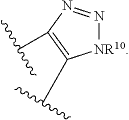

In some embodiments, C is selected from

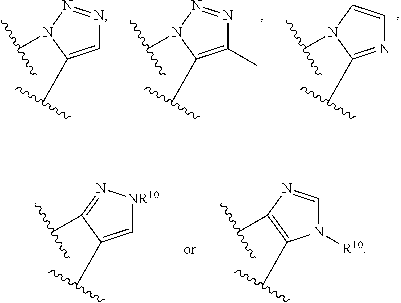

In some such embodiments, R¹⁰ is hydrogen. In some other such embodiments, R is C₁₋₃ alkyl or C₃₋₆ cycloalkyl.

In some embodiments, C is selected from

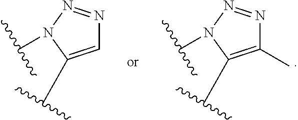

In some embodiments, C is selected from

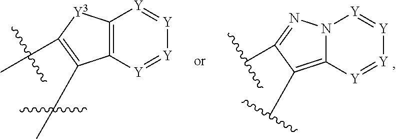

wherein Y³ is selected from O or S. In some such embodiments, Y is a CR⁶. In some other such embodiments, at least one Y is nitrogen. In some embodiments, C is optionally substituted

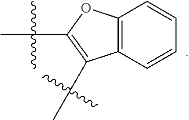 [SMILES: *c1oc2ccccc2c1*]

In some embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other such embodiments, R¹ is optionally substituted aryl.

In some embodiments, m is 1. In some other embodiments, m is 0. In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is R⁶ is hydrogen. In some other embodiments, R is C₁₋₃ alkyl.

In some embodiments, R⁴ is alkyl optionally substituted with halogen. In some other embodiments, R⁴ is hydrogen.

In some embodiments of the compound of Formula (XIV) or (XIVa),

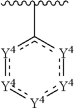

can be

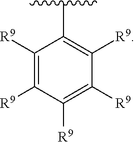

In some embodiments, each of R⁹ is hydrogen. In some other embodiments, at least one R⁹ is selected from C₁₋₃ alkyl or halogen.

In some embodiments,

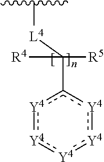

can be

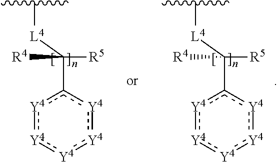

Some embodiments of the compounds of Formula (XIV) are selected from compounds III-1B through III-264B of Table 3B, and pharmaceutically acceptable salts thereof.

Some embodiments of the compounds of Formula (XIV) are selected from IT152, IT193 or IT224 as shown in Table 13.

Formula (XV)

Some embodiments disclosed herein include a compound of Formula (XV) as described above or a pharmaceutically acceptable salt thereof.

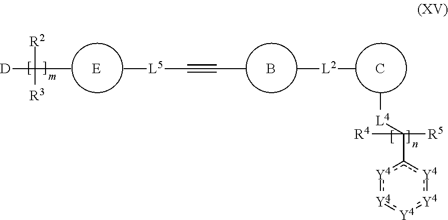

In some embodiments,

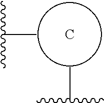

is selected from

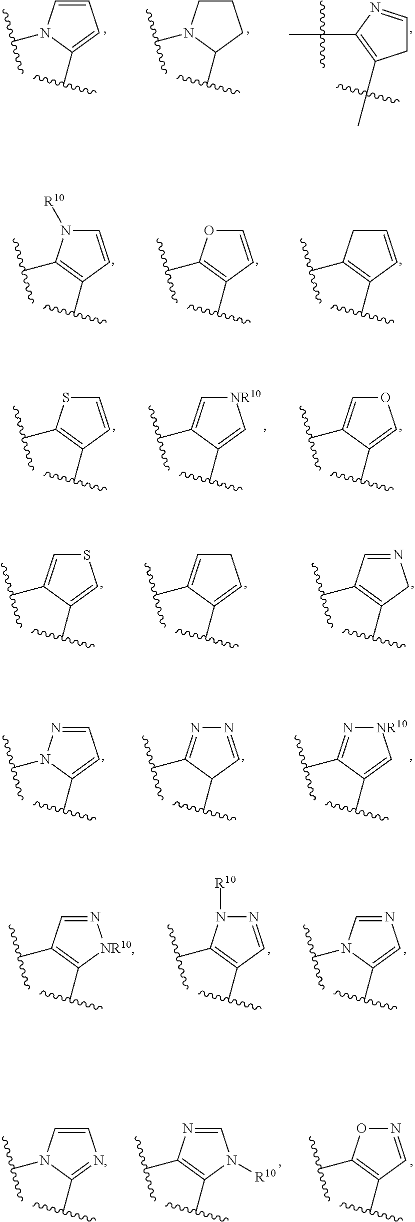

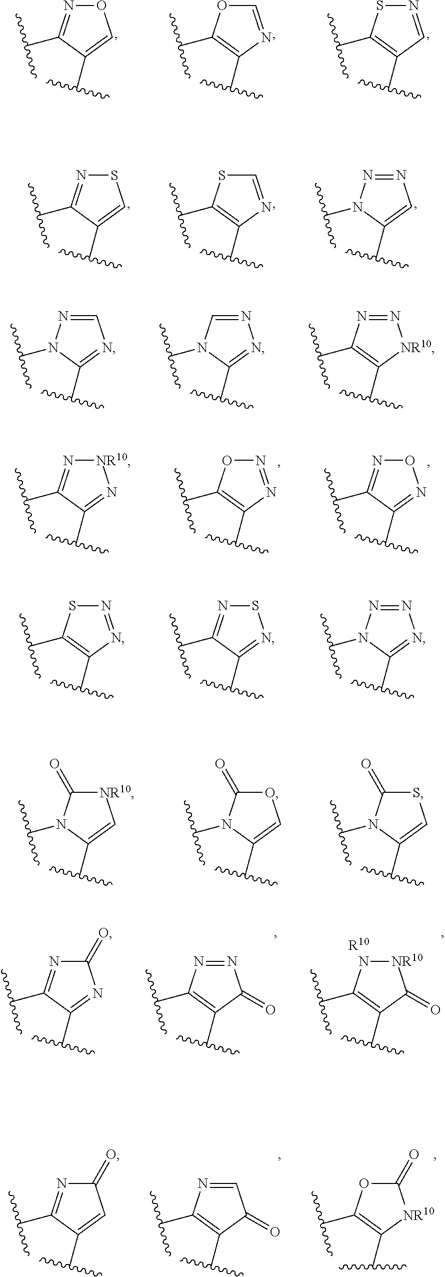

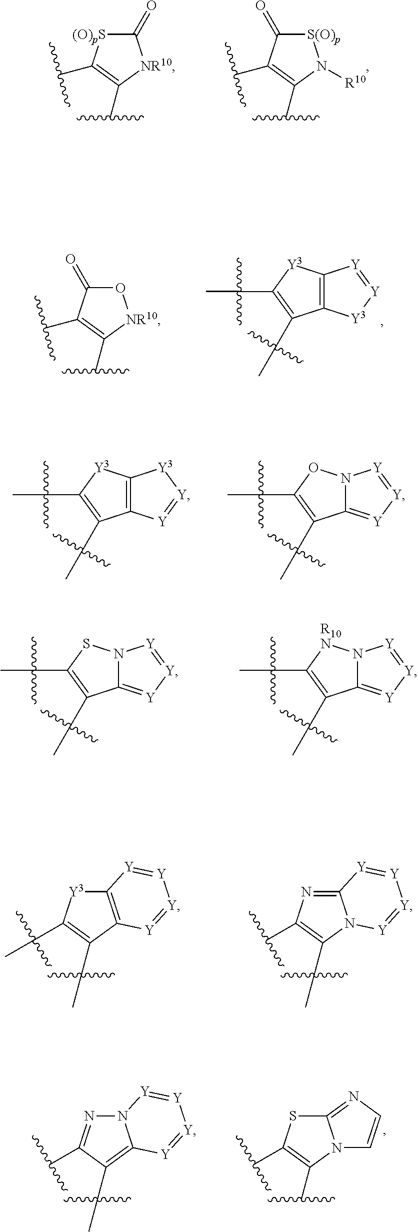

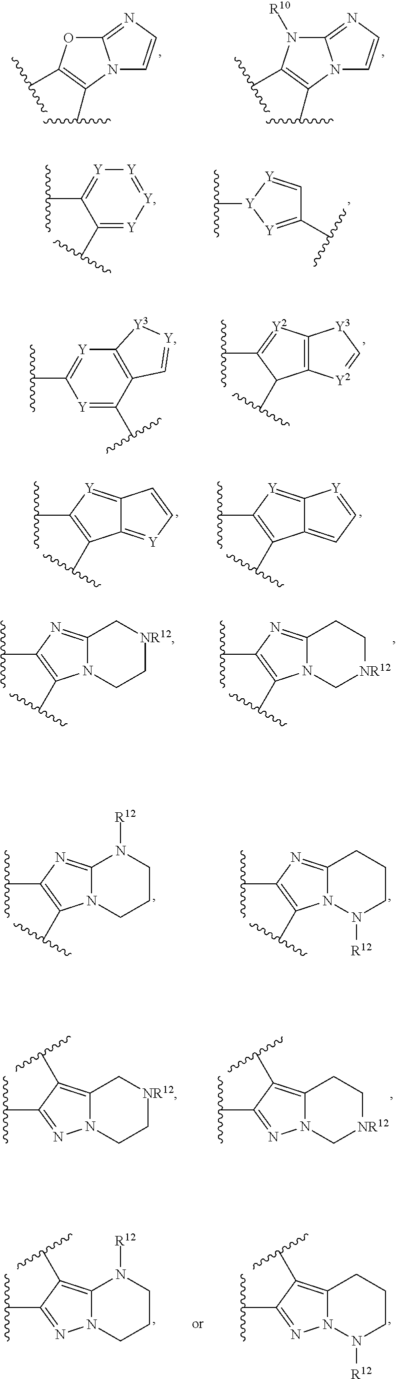

or optionally substituted variants thereof; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido. In some embodiments, ring C cannot be isoxazole or alkyl substituted isoxazole.

In some embodiments, the compound of Formula (XV) is also represented by Formula (XVa):

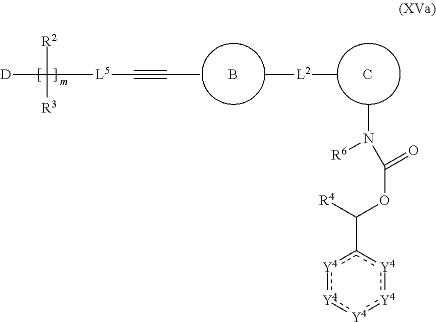

wherein B is selected from the group consisting of

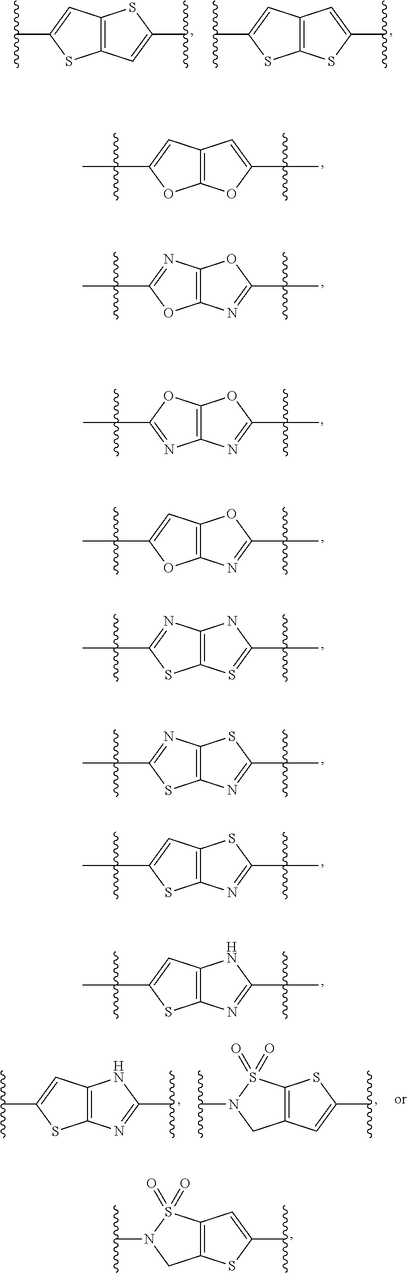

wherein the rings in B are unsubstituted or substituted with one or more substituents selected from alkyl, amino, haloalkyl, halogen, hydroxy, alkoxy, haloalkoxy, cyano, sulfonyl or oxo.

In some embodiments, the ring system in B is independently optionally substituted with one or more substituents selected from the group consisting of alkyl, amino, haloalkyl, halogen, hydroxy, alkoxy, haloalkoxy, cyano, or oxo; E is absent or optionally substituted with one or more substituents selected from the group consisting of alkyl, amino, haloalkyl, halogen, hydroxy, alkoxy, haloalkoxy, cyano or oxo.

In some such embodiments, B is selected from

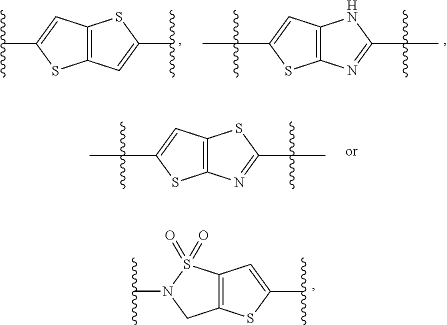 [SMILES: *c1cc2sc(*)cc2s1 ; *c1nc2sc(*)cc2[nH]1 ; *c1cc2sc(*)nc2s1 ; *c1cc2c(s1)CN(*)S2(=O)=O]

each can be optionally substituted. In one embodiment, B is optionally substituted

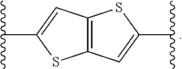 [SMILES: *c1cc2sc(*)cc2s1]

In some embodiments described herein of the compounds of Formula (XV) or (XVa), rings in B are unsubstituted.

In some embodiments described herein of the compounds of Formula (XV) or (XVa), rings in B can be substituted. In some embodiments, rings in B can be substituted with one or more substituents selected from alkyl, amino, haloalkyl, halogen, hydroxy, alkoxy, haloalkoxy, cyano, or oxo. In some embodiments, rings in B can be substituted with one or more sulfonyl groups, for example, methanesulfonyl.

In some embodiments described herein of the compounds of Formula (XV), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

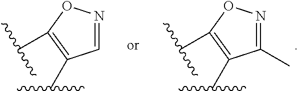

In some embodiments, C is selected from

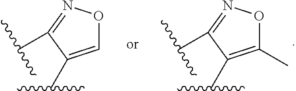

In some embodiments, C is selected from

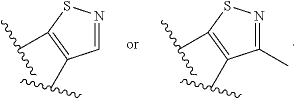

In some embodiments, C is selected from

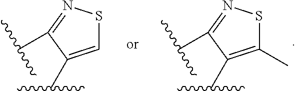

In some embodiments, C is selected from

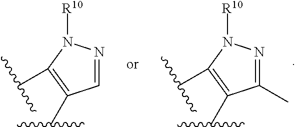

In some embodiments, C is

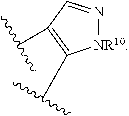

In some embodiments, C is

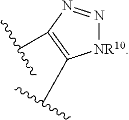

In some embodiments, C is

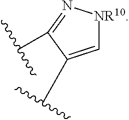

In some embodiments, C is selected from

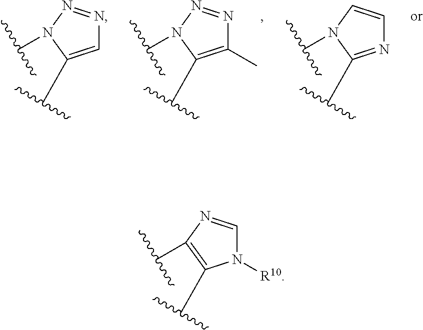

In some such embodiments, R¹⁰ is hydrogen. In some other such embodiments, R¹⁰ is C₁₋₃ alkyl or C₃₋₆ cycloalkyl.

In some embodiments, C is selected from

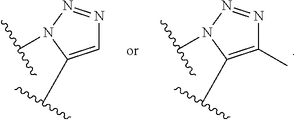

In some embodiments, C is selected from

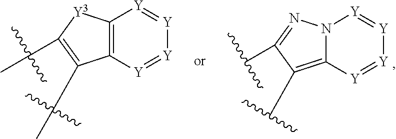

wherein Y³ is selected from O or S. In some such embodiments, Y is a CR⁶. In some other such embodiments, at least one Y is nitrogen. In some embodiments, C is optionally substituted

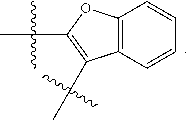 [SMILES: *c1oc2ccccc2c1*]

In some embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other such embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is R⁶ is hydrogen. In some other embodiments, R is C₁₋₃ alkyl.

In some embodiments, R⁴ is alkyl optionally substituted with halogen. In some other embodiments, R⁴ is hydrogen.

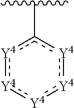

In some embodiments of the compound of Formula (XV) or (XVa), can be

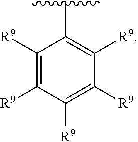

In some embodiments, each of R⁹ is hydrogen. In some other embodiments, at least one R⁹ is selected from C₁₋₃ alkyl or halogen.

In some embodiments,

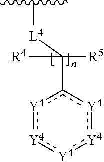

can be

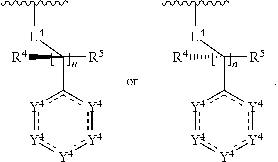

Some embodiments of the compounds of Formula (XV) is selected from IT117, IT145 or IT418 as shown in Table 13.

Formula (XVI)

Some embodiments disclosed herein include a compound of Formula (XVI) as described above or a pharmaceutically acceptable salt thereof.

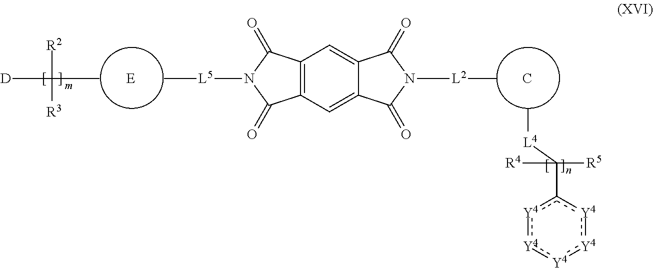

In some embodiments,

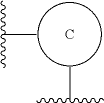

is selected from

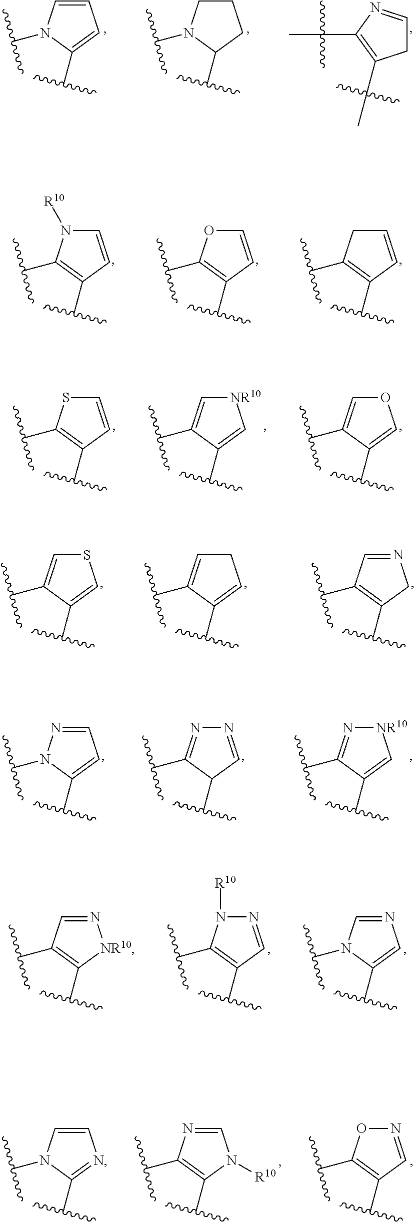

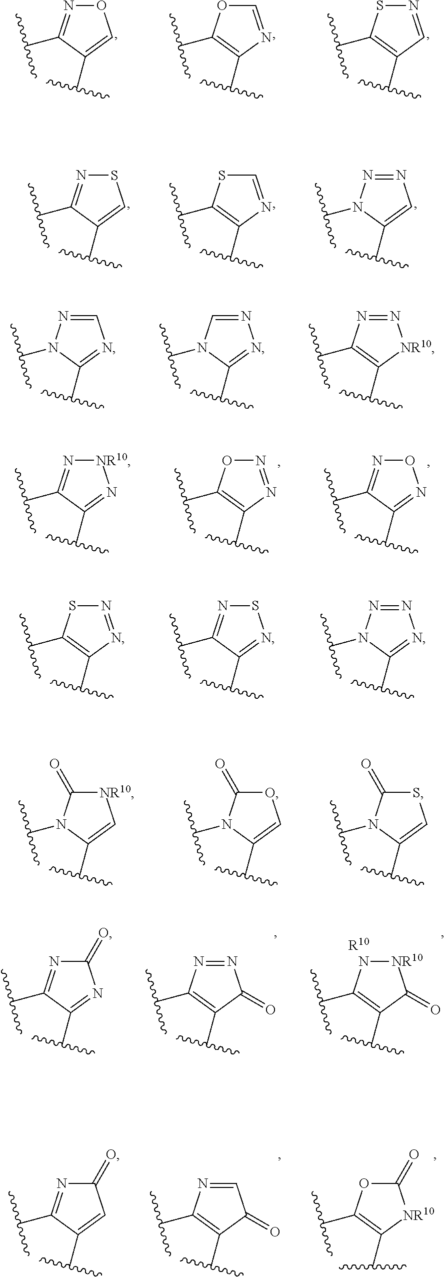

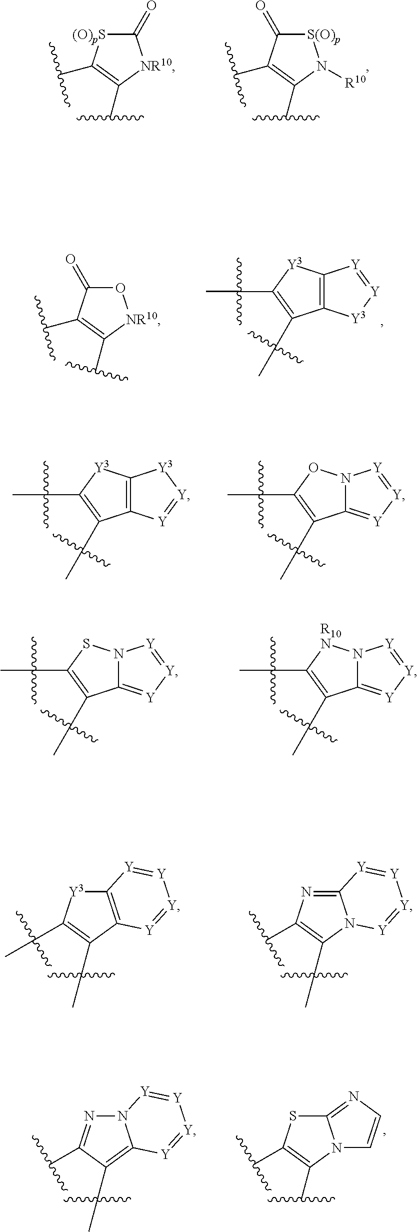

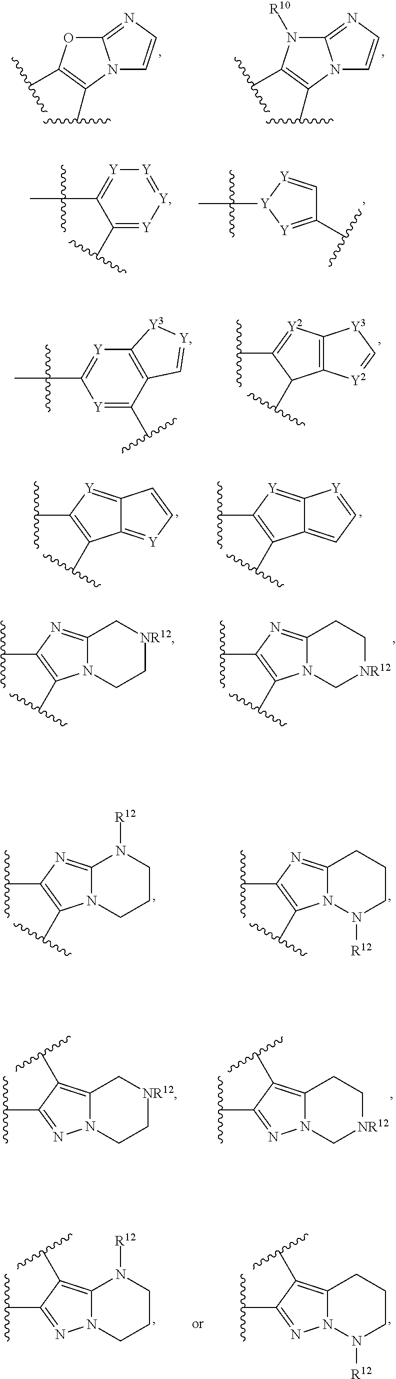

or optionally substituted variants thereof; and each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (XVI) is also represented by Formula (XVIa):

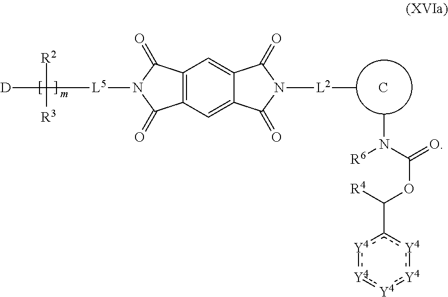

In some embodiments,

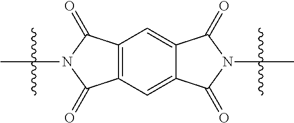 [SMILES: *n1c(=O)c2cc3c(=O)n(*)c(=O)c3cc2c1=O]

is optionally substituted with one or more substituents selected from alkyl, amino, haloalkyl, halogen, hydroxy, alkoxy, haloalkoxy, cyano, or oxo; and E is absent or optionally substituted with one or more substituents selected from alkyl, amino, haloalkyl, halogen, hydroxy, alkoxy, haloalkoxy, cyano, or oxo.

In some embodiments described herein of the compounds of Formula (XVI) or (XVIa),

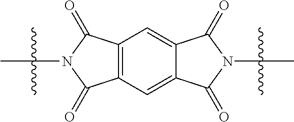 [SMILES: *n1c(=O)c2cc3c(=O)n(*)c(=O)c3cc2c1=O]

is unsubstituted.

In some embodiments described herein of the compounds of Formula (XVI) or (XVIa),

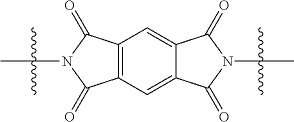 [SMILES: *n1c(=O)c2cc3c(=O)n(*)c(=O)c3cc2c1=O]

can be substituted. In some embodiments,

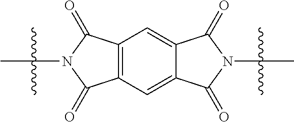 [SMILES: *n1c(=O)c2cc3c(=O)n(*)c(=O)c3cc2c1=O]

can be substituted with one or more substituents selected from alkyl, amino, haloalkyl, halogen, hydroxy, alkoxy, haloalkoxy, cyano, or oxo. In some embodiments,

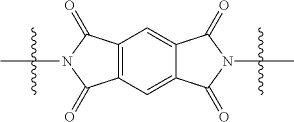 [SMILES: *n1c(=O)c2cc3c(=O)n(*)c(=O)c3cc2c1=O]

can be substituted with one or more sulfonyl groups, for example, methanesulfonyl.

In some embodiments described herein of the compounds of Formula (XVI), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

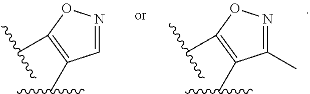

In some embodiments, C is selected from

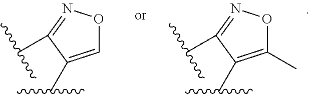

In some embodiments, C is selected from

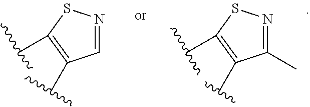

In some embodiments, C is selected from

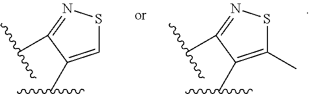

In some embodiments, C is selected from

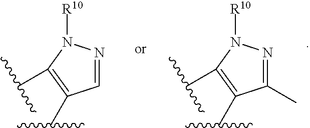

In some embodiments, C is

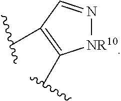

In some embodiments, C is

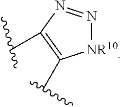

In some embodiments, C is

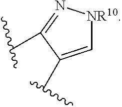

In some embodiments, C is selected from

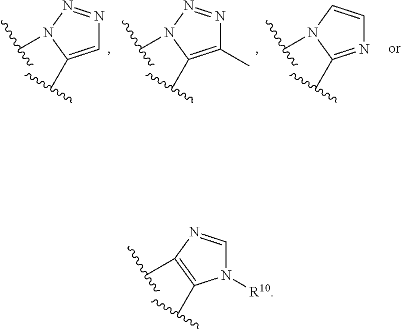

In some such embodiments, R¹⁰ is hydrogen. In some other such embodiments, R¹⁰ is C₁₋₃ alkyl or C₃₋₆ cycloalkyl.

In some embodiments, C is selected from

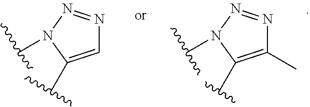

In some embodiments, C is selected from

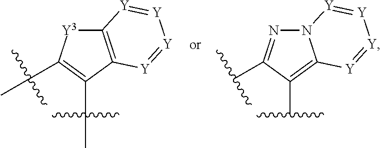

wherein Y³ is selected from O or S. In some such embodiments, Y is a CR⁶. In some other such embodiments, at least one Y is nitrogen. In some embodiments, C is optionally substituted

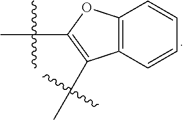 [SMILES: *c1oc2ccccc2c1*]

In some embodiments, D is —C(O)OR¹. In some such embodiments, R¹ is hydrogen or unsubstituted alkyl. In some other such embodiments, R¹ is alkyl substituted with one or more substituents selected from the group consisting of alkoxy, C-amido, O-carboxy, and 6 membered heterocyclyl. In still some other such embodiments, R¹ is optionally substituted aryl.

In some embodiments, m is 0. In some other embodiments, m is 1. In some other embodiments, m is 2.

In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond. In some embodiments, L² is a single bond.

In some embodiments, R⁶ is R⁶ is hydrogen. In some other embodiments, R is C₁₋₃ alkyl.

In some embodiments, R⁴ is alkyl optionally substituted with halogen. In some other embodiments, R⁴ is hydrogen.

In some embodiments of the compound of Formula (XVI) or (XVIa),

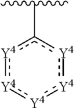

can be

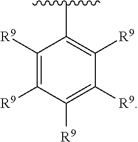

In some embodiments, each of R⁹ is hydrogen. In some other embodiments, at least one R⁹ is selected from C₁₋₃ alkyl or halogen.

In some embodiments,

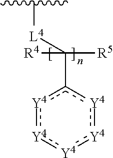

can be

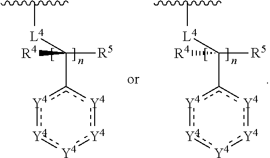

Formula (XVII)

Some embodiments disclosed herein include a compound of Formula (XVII) as described above or a pharmaceutically acceptable salt thereof.

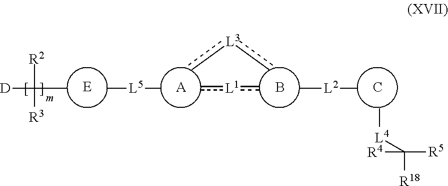

In some embodiments, each of A and B can be an acetylene or selected from the group consisting of

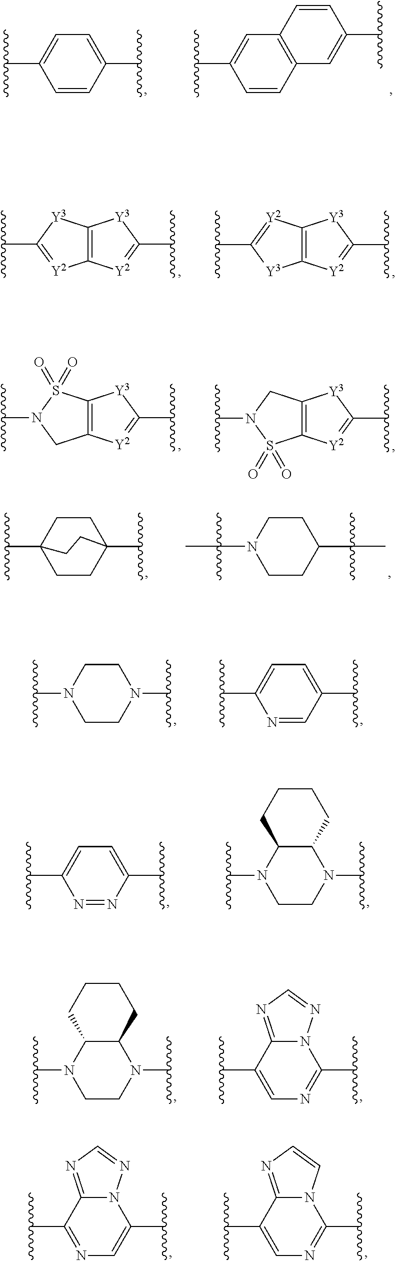

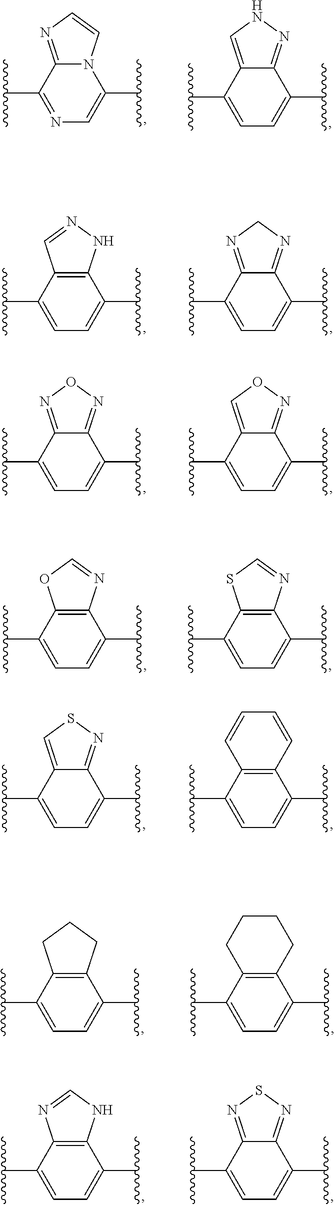

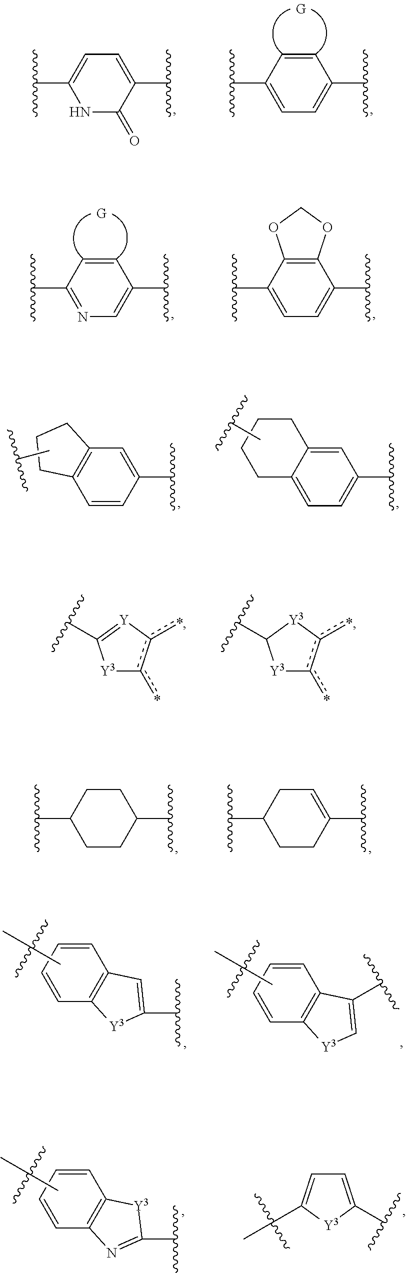

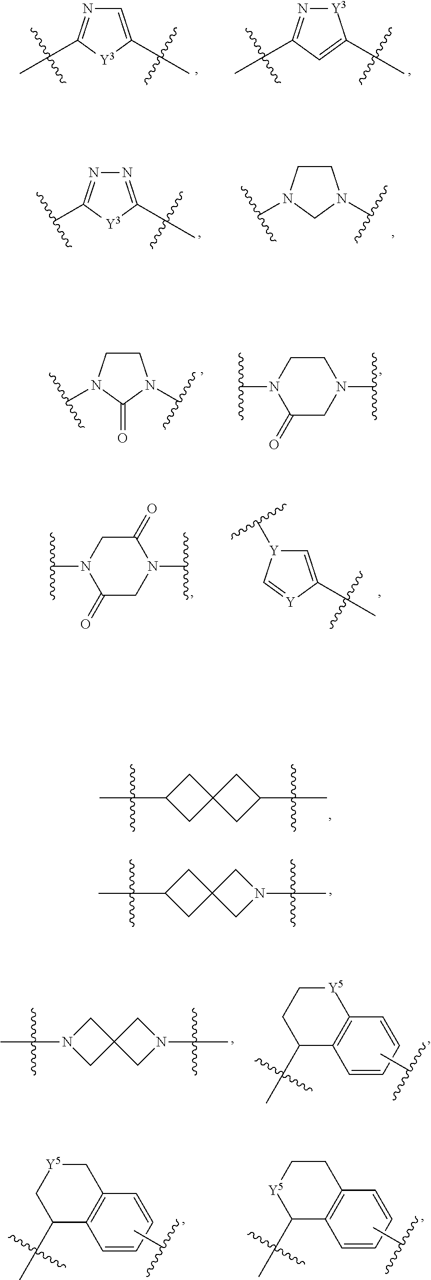

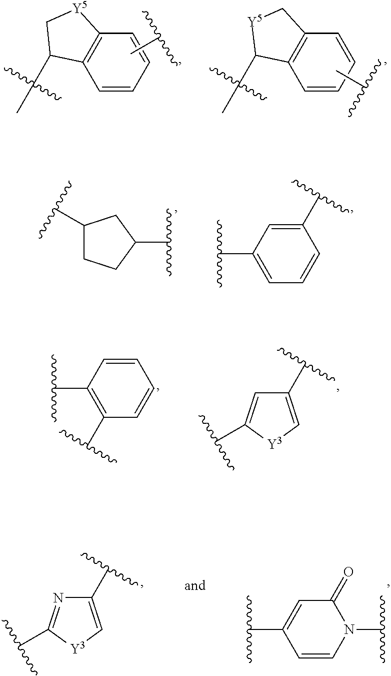

wherein each * is a point of attachment of A or B to L¹ or L³, and wherein the rings in A or B are unsubstituted or substituted with one or more substituents selected from alkyl, amino, haloalkyl, halogen, hydroxy, alkoxy, haloalkoxy, cyano, sulfonyl or oxo;

G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, or oxo;

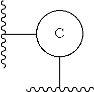

is selected from

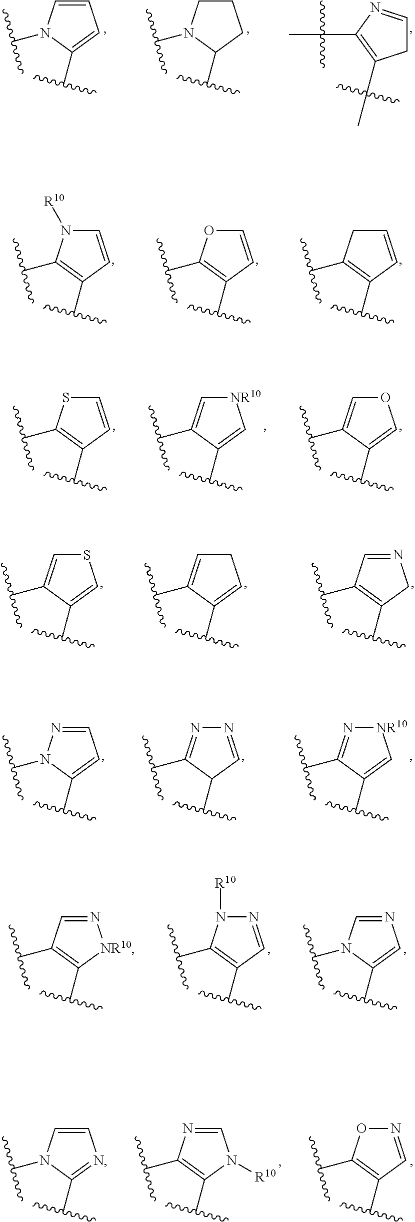

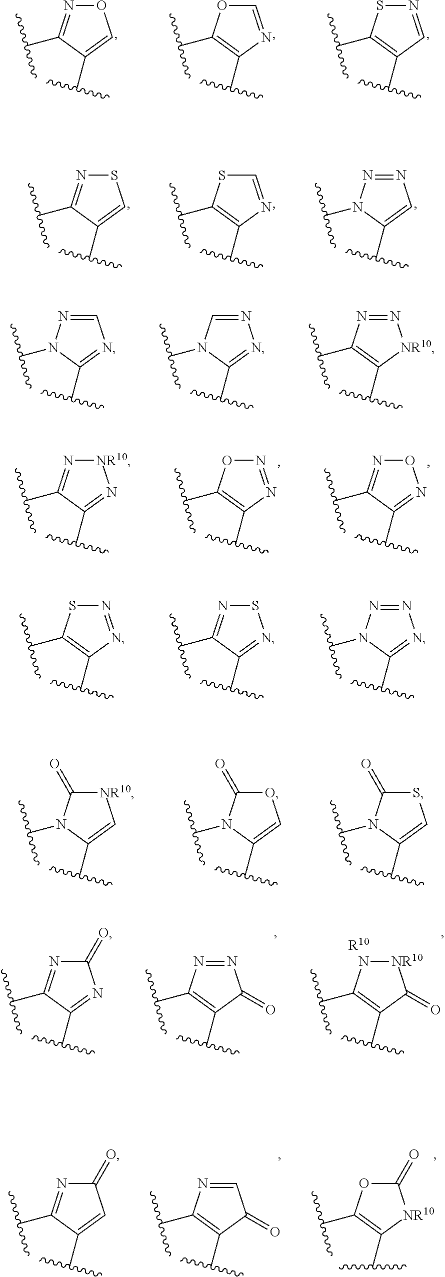

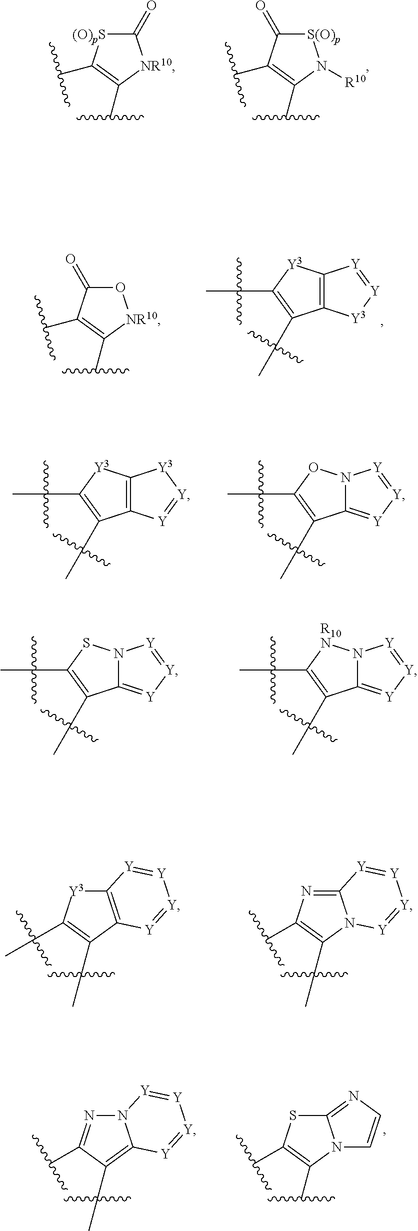

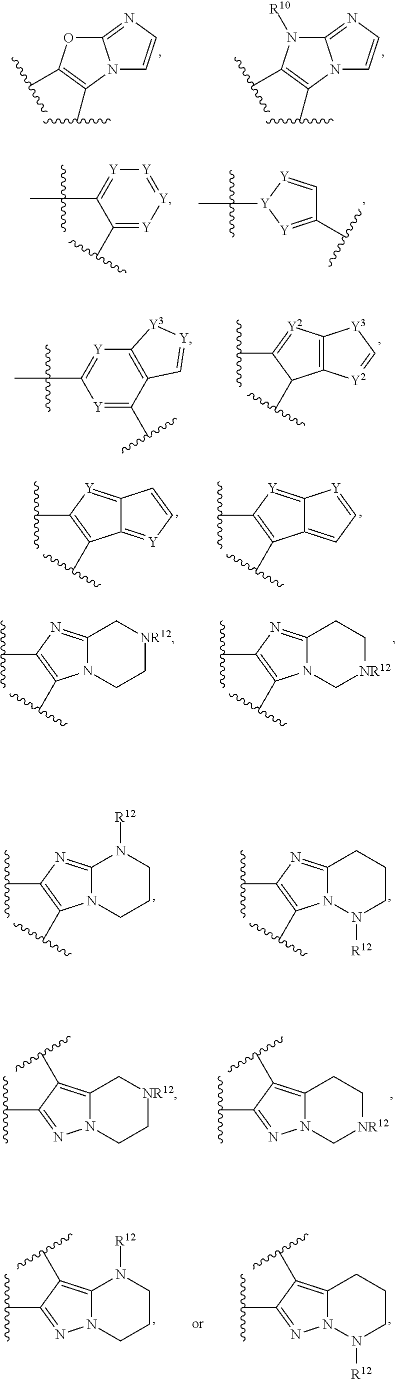

or optionally substituted variants thereof;

each Y² is independently selected from —CH═ or N;

each Y³ is independently selected from C(R⁶)₂, NR⁶, O or S;

each Y⁵ is independently selected from NR⁶, O or S; and

each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

In some embodiments, the compound of Formula (XVII) is also represented by Formula (XIIVa):

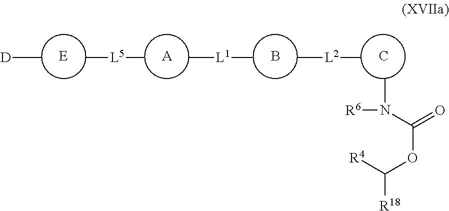

Wherein each of A and B can be independently selected from acetylene,

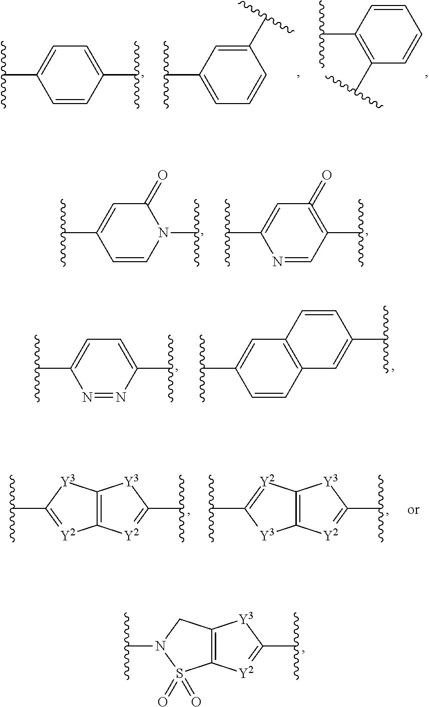

provided that A and B are not both acetylene, and the rings in A or B are unsubstituted or substituted with one or more substituents selected from alkyl, amino, haloalkyl, halogen, hydroxy, alkoxy, haloalkoxy, cyano, sulfonyl or oxo

L¹ is selected from a single bond, a —C(O)— linker, a —CH₂— linker, or a —CH₂O— linker;

L² is selected from a single bond, a —O— linker, a —NH— linker, a —C(O)— linker, a —CH₂— linker, or a —CH₂O— linker; and L⁵ is selected from a single bond or

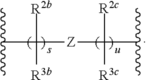

In some embodiments, E is absent.

In some embodiments, E is a phenylene. In some of these embodiments, E is

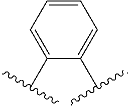 [SMILES: *c1ccccc1*]

In some embodiments, E is a six-membered heteroarylene comprising one or two nitrogen atoms. In any of the embodiments of E, E can be optionally substituted with one or more substituents selected from alkyl, amino, halogen, haloalkyl, hydroxy, alkoxy, haloalkoxy, cyano, or oxo.

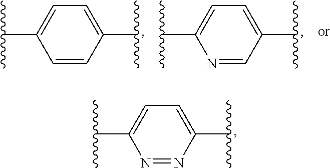

In some embodiments, one of A or B is selected from

and the other A or B is selected from acetylene,

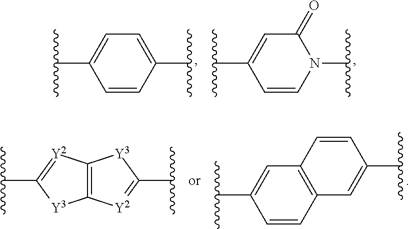

In some of these embodiments, both A and B are

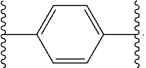 [SMILES: *c1ccc(*)cc1]

In any of the embodiments of A and B, each of the rings in A or B can be unsubstituted or substituted with one or more substituents selected from alkyl, amino, haloalkyl, halogen, hydroxy, alkoxy, haloalkoxy, cyano, sulfonyl or oxo.

In some embodiments described herein of the compounds of Formula (XVII) or (XVIIa), rings in A are unsubstituted.

In some embodiments described herein of the compounds of Formula (XVII) or (XVIIa), rings in B are unsubstituted.

In some embodiments described herein of the compounds of Formula (XVII) or (XVIIa), rings in A can be substituted. In some such embodiments, A can be substituted with one or more substituents selected from alkyl, amino, haloalkyl, halogen, hydroxy, alkoxy, haloalkoxy, cyano, or oxo. In some embodiments, A can be substituted with one or more sulfonyl, for example, methanesulfonyl.

In some embodiments described herein of the compounds of Formula (XVII) or (XVIIa), rings in B can be substituted. In some such embodiments, B can be substituted with one or more substituents selected from alkyl, amino, haloalkyl, halogen, hydroxy, alkoxy, haloalkoxy, cyano, sulfonyl or oxo. In some embodiments, B can be substituted with one or more sulfonyl, for example, methanesulfonyl.

In some embodiments described herein of the compounds of Formula (XVII) or (XVIIa), E can be absent. In some other embodiments, E can be selected from thiazolylene, oxazolylene, triazolylene, pyrazolylene, imidazolylene, thiophenylene, furanylene, pyrrolylene, benzothiazolylene, benzooxazolylene, benzothiophenelylene, benzofuranylene, indolylene, benzoimidazolylene, quinolinylene, isoquinolinylene, phenylene, or pyridylene. In some embodiments, E can be unsubstituted or substituted. In some embodiments, E can be substituted with one or more substituents selected from alkyl, amino, haloalkyl, halogen, hydroxy, alkoxy, haloalkoxy, cyano, or oxo. In some embodiments, E can be substituted with one or more sulfonyl groups, for example, methanesulfonyl.

In some embodiments, C is substituted with one or more one or more substituents selected from C₁₋₃ alkyl optionally substituted with halogen or C₁₋₃ alkoxy; C₁₋₆ alkoxy; C₃₋₆ cycloalkyl; halogen; oxo or cyano. In some other embodiments, C is unsubstituted. In some further embodiments, C is selected from an oxazole, an isoxazole, a thiazole, or an isothiazole, and wherein C is unsubstituted or substituted with one or more substituents selected from C₁₋₃ alkyl optionally substituted with halogen or C₁₋₃ alkoxy; C₁₋₆ alkoxy; C₃₋₆ cycloalkyl; haloge or cyano.

In some embodiments, C is selected from

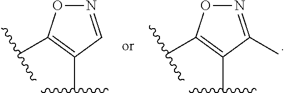

In some embodiments, C is selected from

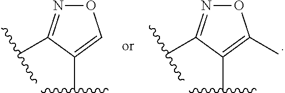

In some embodiments, C is selected from

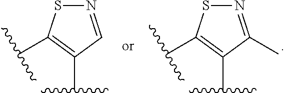

In some embodiments, C is selected from

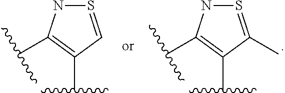

In some embodiments, C is selected from

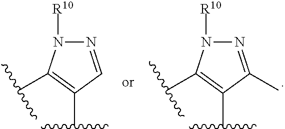

In some embodiments, C is

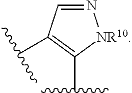

In some embodiments, C is

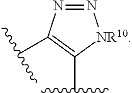

In some embodiments, C is

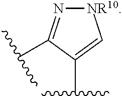

In some embodiments, C is selected from

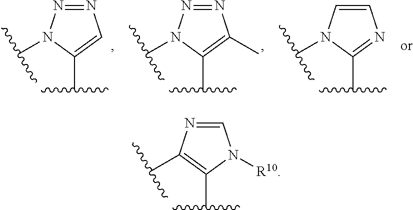

In some such embodiments, R¹⁰ is hydrogen. In some other such embodiments, R¹⁰ is C₁₋₃ alkyl or C₃₋₆ cycloalkyl.

In some embodiments, C is selected from

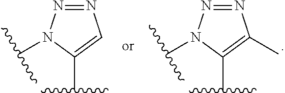

In some embodiments, C is selected from

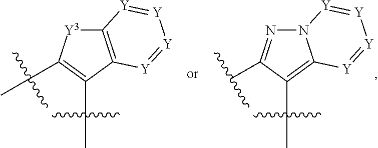

wherein Y³ is selected from O or S. In some such embodiments, Y is a CR⁶. In some other such embodiments, at least one Y is nitrogen. In some embodiments, C is optionally substituted

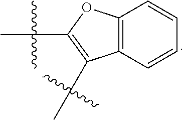 [SMILES: *c1oc2ccccc2c1*]

In some embodiments, D is —C(O)OR¹. In some such embodiments, R¹ is hydrogen.

In some embodiments, m is 0. In some other embodiments, m is 1.

In some embodiments, each of R² and R³ is hydrogen. In some other embodiments, one of R² or R³ is hydrogen and the other R² or R³ is alkyl or aryl. In some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L¹ is a single bond.

In some embodiments, L² is a single bond.

In some embodiments, R⁶ is hydrogen.

In some embodiments, L⁵ is

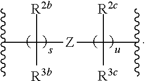

In some of these embodiments, both s and u in L⁵ are 0. In some of these embodiments, L⁵ is —O—.

In some embodiments, R⁴ is alkyl optionally substituted with halogen. In some other embodiments, R⁴ is hydrogen.

In some embodiments, R¹⁸ isselected from C₁₋₃ alkyl.

In some embodiments,

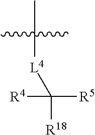

can be selected from

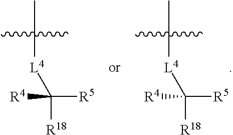

Some embodiments of the compounds of Formula (XVII) is selected from IT493 or IT494 as shown in Table 13.

Some embodiments of the compounds described herein are selected from compounds of Tables 10B, 11A, 11B, 11C and 11D and pharmaceutically acceptable salts thereof.

Some embodiments of the compounds described herein are selected from compounds IT004, IT026-036, IT038-IT045, IT047-IT049, IT052, IT055, IT061, IT064, IT068, IT069, IT072-IT081, IT093, IT094, IT096, IT102, IT105, IT112, IT113, IT125, IT146, IT225, IT233, IT234, IT235, IT280, IT496 and IT499 as shown in Table 13.

Exemplary Compounds

In some embodiments, compounds of Formula (I) are selected from the following compounds as listed in Table 1.

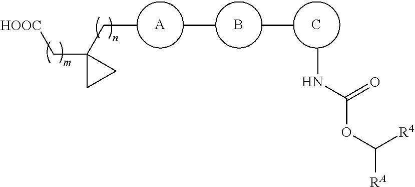

TABLE 1

| Cmpd # | m | n | A | B | C | R⁴ | R^{A} |
|---|---|---|---|---|---|---|---|
| I-1 | 1 | 0 | acetylene | phen-1,4-ylene | 3-methyl isothiazole-4,5-diyl | CH₃ | phenyl |
| I-2 | 0 | 1 | acetylene | phen-1,4-ylene | 3-methyl isothiazole-4,5-diyl | CH₃ | phenyl |
| I-3 | 0 | 1 | phen-1,4-ylene | acetylene | 3-methyl isooxazole-4,5-diyl | CH₃ | phenyl |
| I-4 | 1 | 0 | naphthalen-2,6-ylene | acetylene | 3-methyl isooxazole-4,5-diyl | CH₃ | 2-chloro-phenyl |
| I-5 | 0 | 1 | naphthalen-2,6-ylene | acetylene | 3-methyl isooxazole-4,5-diyl | CH₃ | phenyl |
| I-6 | 1 | 0 | phen-1,4-ylene | acetylene | 3-methyl isooxazole-4,5-diyl | CH₃ | 2-chloro-phenyl |
| I-7 | 1 | 0 | acetylene | phen-1,4-ylene | 3-methyl isothiazole-4,5-diyl | CH₃ | phenyl |
| I-8 | 1 | 0 | phen-1,4-ylene | acetylene | 3-methyl isothiazole-4,5-diyl | CH₃ | phenyl |

Note:
ring C connects to B through the 5-yl position.

In some embodiments, compounds of Formula (II) are selected from the following compounds as listed in Table 2.

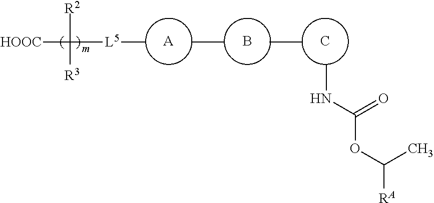

TABLE 2

| Cmpd # | m | R²/R³ | L⁵ | A | B | C | R^{A} |
|---|---|---|---|---|---|---|---|
| II-1 | 1 | 1,1-cyclopropyl | —NH— | phen-1,4-ylene | acetylene | 3-methyl isooxazol-4,5-diyl | 2-chloro-phenyl |
| II-2 | 0 | 1,1-cyclopropyl | —C(O)NH—* | phen-1,4-ylene | acetylene | 3-methyl isooxazole-4,5-diyl | 2-chloro-phenyl |
| II-3 | 0 | 1,1-cyclopropyl | —NH— | naphthalen-2,6-ylene | acetylene | 3-methyl isooxazole-4,5-diyl | phenyl |
| II-4 | 1 | 1,1-cyclopropyl | —C(O)NH—* | naphthalen-2,6-ylene | acetylene | 3-methyl isothiazole-4,5-diyl | phenyl |
| II-5 | 0 | 1,1-cyclopropyl | —NH— | phen-1,4-ylene | acetylene | 3-methyl isothiazole-4,5-diyl | 2-chloro-phenyl |
| II-6 | 1 | 1,1-cyclopropyl | —C(O)NH—* | phen-1,4-ylene | acetylene | 3-methyl isooxazole-4,5-diyl | 2-chloro-phenyl |
| II-7 | 0 | n/a | acetylene | phen-1,4-ylene | phen-1,4-ylene | 3-methyl isooxazole-4,5-diyl | phenyl |
| II-8 | 0 | n/a | acetylene | phen-1,4-ylene | phen-1,4-ylene | 3-methyl isooxazole-4,5-diyl | 2-chloro-phenyl |

Note:
*indicates the position of L⁵ connection to ring A. Ring C connects to B through the 5-yl position.

In some embodiments, compounds of Formula (II) are also represented by

Formula (II-A) and are selected from the following compounds as listed in Table 2A.

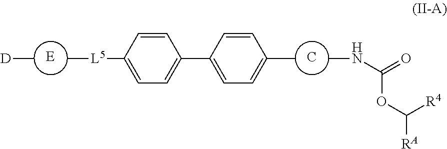

TABLE 2A

| Cmpd # II- | 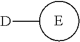 | 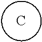 | * | L⁵ | R⁴ | R^{A} |
|---|---|---|---|---|---|---|
| 1A | 2-carboxy-phenyl | 3-methyl isooxazol-4,5-diyl | 5 | —S(O)₂— | methyl | phenyl |
| 2A | 2-carboxy-phenyl | 3-methyl isooxazol-4,5-diyl | 5 | —NH— | methyl | phenyl |
| 3A | 2-carboxy-phenyl | isooxazol-3,4-diyl | 3 | —O— | H | phenyl |
| 4A | 2-carboxy-phenyl | isooxazol-3,4-diyl | 3 | —S(O)₂— | H | phenyl |
| 5A | 2-carboxy-phenyl | isooxazol-3,4-diyl | 3 | —NH— | H | phenyl |
| 6A | 2-carboxy-phenyl | isooxazol-3,4-diyl | 3 | —O— | H | 2-methyl-phenyl |
| 7A | 2-carboxy-phenyl | isooxazol-3,4-diyl | 3 | —S(O)₂— | H | 2-methyl-phenyl |
| 8A | 2-carboxy-phenyl | isooxazol-3,4-diyl | 3 | —NH— | H | 2-methyl-phenyl |
| 9A | 2-carboxy-phenyl | isooxazol-3,4-diyl | 3 | —O— | H | 2-chloro-phenyl |
| 10A | 2-carboxy-phenyl | isooxazol-3,4-diyl | 3 | —S(O)₂— | H | 2-chloro-phenyl |
| 11A | 2-carboxy-phenyl | isooxazol-3,4-diyl | 3 | —NH— | H | 2-chloro-phenyl |
| 12A | 2-carboxy-phenyl | isothiazol-3,4-diyl | 3 | —O— | H | phenyl |
| 13A | 2-carboxy-phenyl | isothiazol-3,4-diyl | 3 | —S(O)₂— | H | phenyl |
| 14A | 2-carboxy-phenyl | isothiazol-3,4-diyl | 3 | —NH— | H | phenyl |
| 15A | 2-carboxy-phenyl | isothiazol-3,4-diyl | 3 | —O— | H | 2-methyl-phenyl |
| 16A | 2-carboxy-phenyl | isothiazol-3,4-diyl | 3 | —S(O)₂— | H | 2-methyl-phenyl |
| 17A | 2-carboxy-phenyl | isothiazol-3,4-diyl | 3 | —NH— | H | 2-methyl-phenyl |
| 18A | 2-carboxy-phenyl | isothiazol-3,4-diyl | 3 | —O— | H | 2-chloro-phenyl |
| 19A | 2-carboxy-phenyl | isothiazol-3,4-diyl | 3 | —S(O)₂— | H | 2-chloro-phenyl |
| 20A | 2-carboxy-phenyl | isothiazol-3,4-diyl | 3 | —NH— | H | 2-chloro-phenyl |
| 21A | 2-carboxy-phenyl | isothiazol-4,5-diyl | 5 | —O— | H | phenyl |
| 22A | 2-carboxy-phenyl | isothiazol-4,5-diyl | 5 | —S(O)₂— | H | phenyl |
| 23A | 2-carboxy-phenyl | isothiazol-4,5-diyl | 5 | —NH— | H | phenyl |
| 24A | 2-carboxy-phenyl | isothiazol-4,5-diyl | 5 | —O— | H | 2-methyl-phenyl |
| 25A | 2-carboxy-phenyl | isothiazol-4,5-diyl | 5 | —S(O)₂— | H | 2-methyl-phenyl |
| 26A | 2-carboxy-phenyl | isothiazol-4,5-diyl | 5 | —NH— | H | 2-methyl-phenyl |
| 27A | 2-carboxy-phenyl | isothiazol-4,5-diyl | 5 | —O— | H | 2-chloro-phenyl |
| 28A | 2-carboxy-phenyl | isothiazol-4,5-diyl | 5 | —S(O)₂— | H | 2-chloro-phenyl |
| 29A | 2-carboxy-phenyl | isothiazol-4,5-diyl | 5 | —NH— | H | 2-chloro-phenyl |
| 30A | 2-carboxy-phenyl | 1,2,3-triazol-4,5-diyl | 5 | —S(O)₂— | H | phenyl |
| 31A | 2-carboxy-phenyl | 1,2,3-triazol-4,5-diyl | 5 | —NH— | H | phenyl |
| 32A | 2-carboxy-phenyl | 1,2,3-triazol-4,5-diyl | 5 | —S(O)₂— | H | 2-methyl-phenyl |
| 33A | 2-carboxy-phenyl | 1,2,3-triazol-4,5-diyl | 5 | —NH— | H | 2-methyl-phenyl |
| 34A | 2-carboxy-phenyl | 1,2,3-triazol-4,5-diyl | 5 | —S(O)₂— | H | 2-chloro-phenyl |
| 35A | 2-carboxy-phenyl | 1,2,3-triazol-4,5-diyl | 5 | —NH— | H | 2-chloro-phenyl |
| 36A | 2-carboxy-phenyl | 1,2,3-triazol-1,5-diyl | 1 | —S(O)₂— | H | phenyl |
| 37A | 2-carboxy-phenyl | 1,2,3-triazol-1,5-diyl | 1 | —NH— | H | phenyl |
| 38A | 2-carboxy-phenyl | 1,2,3-triazol-1,5-diyl | 1 | —S(O)₂— | H | 2-methyl-phenyl |
| 39A | 2-carboxy-phenyl | 1,2,3-triazol-1,5-diyl | 1 | —NH— | H | 2-methyl-phenyl |
| 40A | 2-carboxy-phenyl | 1,2,3-triazol-1,5-diyl | 1 | —S(O)₂— | H | 2-chloro-phenyl |
| 41A | 2-carboxy-phenyl | 1,2,3-triazol-1,5-diyl | 1 | —NH— | H | 2-chloro-phenyl |
| 42A | 2-carboxy-phenyl | 1H-pyrazol-4,5-diyl | 4 | —S(O)₂— | H | phenyl |
| 43A | 2-carboxy-phenyl | 1H-pyrazol-4,5-diyl | 4 | —NH— | H | phenyl |
| 44A | 2-carboxy-phenyl | 1H-pyrazol-4,5-diyl | 4 | —S(O)₂— | H | 2-methyl-phenyl |
| 45A | 2-carboxy-phenyl | 1H-pyrazol-4,5-diyl | 4 | —NH— | H | 2-methyl-phenyl |
| 46A | 2-carboxy-phenyl | 1H-pyrazol-4,5-diyl | 4 | —S(O)₂— | H | 2-chloro-phenyl |
| 47A | 2-carboxy-phenyl | 1H-pyrazol-4,5-diyl | 4 | —NH— | H | 2-chloro-phenyl |
| 48A | 2-carboxy-phenyl | 3-methyl isooxazol-4,5-diyl | 5 | —O— | H | phenyl |
| 49A | 2-carboxy-phenyl | 3-methyl isooxazol-4,5-diyl | 5 | —S(O)₂— | H | phenyl |
| 50A | 2-carboxy-phenyl | 3-methyl isooxazol-4,5-diyl | 5 | —NH— | H | phenyl |
| 51A | 2-carboxy-phenyl | 3-methyl isooxazol-4,5-diyl | 5 | —O— | H | 2-methyl-phenyl |
| 52A | 2-carboxy-phenyl | 3-methyl isooxazol-4,5-diyl | 5 | —S(O)₂— | H | 2-methyl-phenyl |
| 53A | 2-carboxy-phenyl | 3-methyl isooxazol-4,5-diyl | 5 | —NH— | H | 2-methyl-phenyl |
| 54A | 2-carboxy-phenyl | 3-methyl isooxazol-4,5-diyl | 5 | —O— | H | 2-chloro-phenyl |
| 55A | 2-carboxy-phenyl | 3-methyl isooxazol-4,5-diyl | 5 | —S(O)₂— | H | 2-chloro-phenyl |
| 56A | 2-carboxy-phenyl | 3-methyl isooxazol-4,5-diyl | 5 | —NH— | H | 2-chloro-phenyl |
| 57A | 2-carboxy-phenyl | isooxazol-3,4-diyl | 3 | —O— | methyl | phenyl |
| 58A | 2-carboxy-phenyl | isooxazol-3,4-diyl | 3 | —S(O)₂— | methyl | phenyl |
| 59A | 2-carboxy-phenyl | isooxazol-3,4-diyl | 3 | —NH— | methyl | phenyl |
| 60A | 2-carboxy-phenyl | isothiazol-4,5-diyl | 5 | —O— | methyl | phenyl |
| 61A | 2-carboxy-phenyl | isothiazol-4,5-diyl | 5 | —S(O)₂— | methyl | phenyl |
| 62A | 2-carboxy-phenyl | isothiazol-4,5-diyl | 5 | —NH— | methyl | phenyl |
| 63A | 2-carboxy-phenyl | 1,2,3-triazol-4,5-diyl | 5 | —S(O)₂— | methyl | phenyl |
| 64A | 2-carboxy-phenyl | 1,2,3-triazol-4,5-diyl | 5 | —NH— | methyl | phenyl |
| 65A | 2-carboxy-phenyl | 1,2,3-triazol-1,5-diyl | 1 | —S(O)₂— | methyl | phenyl |
| 66A | 2-carboxy-phenyl | 1,2,3-triazol-1,5-diyl | 1 | —NH— | methyl | phenyl |
| 67A | 4-carboxy-pyridin-3-yl | isothiazol-3,4-diyl | 3 | —O— | methyl | phenyl |
| 68A | 2-carboxy-pyridin-3-yl | isothiazol-3,4-diyl | 3 | —O— | methyl | phenyl |
| 69A | 3-carboxy-pyridin-4-yl | isothiazol-3,4-diyl | 3 | —O— | methyl | phenyl |
| 70A | 3-carboxy-pyridin-2-yl | isothiazol-3,4-diyl | 3 | —O— | methyl | phenyl |
| 71A | 2-carboxy-phenyl | isooxazol-3,4-diyl | 3 | —O— | methyl | 2-chloro-phenyl |
| 72A | 2-carboxy-phenyl | isooxazol-3,4-diyl | 3 | —S(O)₂— | methyl | 2-chloro-phenyl |
| 73A | 2-carboxy-phenyl | isooxazol-3,4-diyl | 3 | —NH— | methyl | 2-chloro-phenyl |
| 74A | 2-carboxy-phenyl | isothiazol-3,4-diyl | 3 | —O— | methyl | phenyl |
| 75A | 2-carboxy-phenyl | isothiazol-3,4-diyl | 3 | —S(O)₂— | methyl | phenyl |
| 76A | 2-carboxy-phenyl | isothiazol-3,4-diyl | 3 | —NH— | methyl | phenyl |
| 77A | 2-carboxy-phenyl | isothiazol-3,4-diyl | 3 | —O— | methyl | 2-methyl-phenyl |
| 78A | 2-carboxy-phenyl | isothiazol-3,4-diyl | 3 | —S(O)₂— | methyl | 2-methyl-phenyl |
| 79A | 2-carboxy-phenyl | isothiazol-3,4-diyl | 3 | —NH— | methyl | 2-methyl-phenyl |
| 80A | 2-carboxy-phenyl | isothiazol-3,4-diyl | 3 | —O— | methyl | 2-chloro-phenyl |
| 81A | 2-carboxy-phenyl | isothiazol-3,4-diyl | 3 | —S(O)₂— | methyl | 2-chloro-phenyl |
| 82A | 2-carboxy-phenyl | isothiazol-3,4-diyl | 3 | —NH— | methyl | 2-chloro-phenyl |
| 83A | 2-carboxy-phenyl | isothiazol-4,5-diyl | 5 | —O— | methyl | 2-methyl-phenyl |
| 84A | 2-carboxy-phenyl | isothiazol-4,5-diyl | 5 | —S(O)₂— | methyl | 2-methyl-phenyl |
| 85A | 2-carboxy-phenyl | isothiazol-4,5-diyl | 5 | —NH— | methyl | 2-methyl-phenyl |
| 86A | 2-carboxy-phenyl | isothiazol-4,5-diyl | 5 | —O— | methyl | 2-chloro-phenyl |
| 87A | 2-carboxy-phenyl | isothiazol-4,5-diyl | 5 | —S(O)₂— | methyl | 2-chloro-phenyl |
| 88A | 2-carboxy-phenyl | isothiazol-4,5-diyl | 5 | —NH— | methyl | 2-chloro-phenyl |
| 89A | 2-carboxy-phenyl | 1,2,3-triazol-4,5-diyl | 5 | —S(O)₂— | methyl | 2-methyl-phenyl |
| 90A | 2-carboxy-phenyl | 1,2,3-triazol-4,5-diyl | 5 | —NH— | methyl | 2-methyl-phenyl |
| 91A | 2-carboxy-phenyl | 1,2,3-triazol-4,5-diyl | 5 | —S(O)₂— | methyl | 2-chloro-phenyl |
| 92A | 2-carboxy-phenyl | 1,2,3-triazol-4,5-diyl | 5 | —NH— | methyl | 2-chloro-phenyl |
| 93A | 2-carboxy-phenyl | 1,2,3-triazol-1,5-diyl | 1 | —S(O)₂— | methyl | 2-chloro-phenyl |
| 94A | 2-carboxy-phenyl | 1,2,3-triazol-1,5-diyl | 1 | —NH— | methyl | 2-chloro-phenyl |
| 95A | 2-carboxy-phenyl | 1H-pyrazol-4,5-diyl | 4 | —S(O)₂— | methyl | phenyl |
| 96A | 2-carboxy-phenyl | 1H-pyrazol-4,5-diyl | 4 | —NH— | methyl | phenyl |
| 97A | 2-carboxy-phenyl | 1H-pyrazol-4,5-diyl | 4 | —S(O)₂— | methyl | 2-methyl-phenyl |
| 98A | 2-carboxy-phenyl | 1H-pyrazol-4,5-diyl | 4 | —NH— | methyl | 2-methyl-phenyl |
| 99A | 2-carboxy-phenyl | 1H-pyrazol-4,5-diyl | 4 | —S(O)₂— | methyl | 2-chloro-phenyl |
| 100A | 2-carboxy-phenyl | 1H-pyrazol-4,5-diyl | 4 | —NH— | methyl | 2-chloro-phenyl |
| 101A | 4-carboxy-pyridin-3-yl | isothiazol-3,4-diyl | 3 | —O— | methyl | 2-methyl-phenyl |
| 102A | 2-carboxy-pyridin-3-yl | isothiazol-3,4-diyl | 3 | —O— | methyl | 2-methyl-phenyl |
| 103A | 3-carboxy-pyridin-4-yl | isothiazol-3,4-diyl | 3 | —O— | methyl | 2-methyl-phenyl |
| 104A | 3-carboxy-pyridin-2-yl | isothiazol-3,4-diyl | 3 | —O— | methyl | 2-methyl-phenyl |
| 105A | 2-carboxy-phenyl | 3-methyl isooxazol-4,5-diyl | 5 | —O— | methyl | 2-methyl-phenyl |
| 106A | 2-carboxy-phenyl | 3-methyl isooxazol-4,5-diyl | 5 | —S(O)₂— | methyl | 2-methyl-phenyl |
| 107A | 2-carboxy-phenyl | 3-methyl isooxazol-4,5-diyl | 5 | NH— | methyl | 2-methyl-phenyl |
| 108A | 2-carboxy-phenyl | 3-methyl isooxazol-4,5-diyl | 5 | —O— | methyl | 2-chloro-phenyl |
| 109A | 2-carboxy-phenyl | 3-methyl isooxazol-4,5-diyl | 5 | —S(O)₂— | methyl | 2-chloro-phenyl |
| 110A | 2-carboxy-phenyl | 3-methyl isooxazol-4,5-diyl | 5 | —NH— | methyl | 2-chloro-phenyl |
| 111A | 2-carboxy-phenyl | isooxazol-4,5-diyl | 5 | —O— | methyl | phenyl |
| 112A | 2-carboxy-phenyl | isooxazol-4,5-diyl | 5 | —S(O)₂— | methyl | phenyl |
| 113A | 2-carboxy-phenyl | isooxazol-4,5-diyl | 5 | —NH— | methyl | phenyl |
| 114A | 2-carboxy-phenyl | 5-methyl isooxazol-3,4-diyl | 3 | —O— | H | phenyl |
| 115A | 2-carboxy-phenyl | 5-methyl isooxazol-3,4-diyl | 3 | —S(O)₂— | H | phenyl |
| 116A | 2-carboxy-phenyl | 5-methyl isooxazol-3,4-diyl | 3 | —NH— | H | phenyl |
| 117A | 2-carboxy-phenyl | 5-methyl isooxazol-3,4-diyl | 3 | —O— | H | 2-methyl-phenyl |
| 18A | 2-carboxy-phenyl | 5-methyl isooxazol-3,4-diyl | 3 | —S(O)₂— | H | 2-methyl-phenyl |
| 119A | 2-carboxy-phenyl | 5-methyl isooxazol-3,4-diyl | 3 | —NH— | H | 2-methyl-phenyl |
| 120A | 2-carboxy-phenyl | 5-methyl isooxazol-3,4-diyl | 3 | —O— | H | 2-chloro-phenyl |
| 121A | 2-carboxy-phenyl | 5-methyl isooxazol-3,4-diyl | 3 | —S(O)₂— | H | 2-chloro-phenyl |
| 122A | 2-carboxy-phenyl | 5-methyl isooxazol-3,4-diyl | 3 | —NH— | H | 2-chloro-phenyl |
| 123A | 2-carboxy-phenyl | 5-methyl isothiazol-3,4-diyl | 3 | —O— | H | phenyl |
| 124A | 2-carboxy-phenyl | 5-methyl isothiazol-3,4-diyl | 3 | —S(O)₂— | H | phenyl |
| 125A | 2-carboxy-phenyl | 5-methyl isothiazol-3,4-diyl | 3 | —NH— | H | phenyl |
| 126A | 2-carboxy-phenyl | 5-methyl isothiazol-3,4-diyl | 3 | —O— | H | 2-methyl-phenyl |
| 127A | 2-carboxy-phenyl | 5-methyl isothiazol-3,4-diyl | 3 | —S(O)₂— | H | 2-methyl-phenyl |
| 128A | 2-carboxy-phenyl | 5-methyl isothiazol-3,4-diyl | 3 | —NH— | H | 2-methyl-phenyl |
| 129A | 2-carboxy-phenyl | 5-methyl isothiazol-3,4-diyl | 3 | —O— | H | 2-chloro-phenyl |
| 130A | 2-carboxy-phenyl | 5-methyl isothiazol-3,4-diyl | 3 | —S(O)₂— | H | 2-chloro-phenyl |
| 131A | 2-carboxy-phenyl | 5-methyl isothiazol-3,4-diyl | 3 | —NH— | H | 2-chloro-phenyl |
| 132A | 2-carboxy-phenyl | 3-methyl isothiazol-4,5-diyl | 5 | —O— | H | phenyl |
| 133A | 2-carboxy-phenyl | 3-methyl isothiazol-4,5-diyl | 5 | —S(O)₂— | H | phenyl |
| 134A | 2-carboxy-phenyl | 3-methyl isothiazol-4,5-diyl | 5 | —NH— | H | phenyl |
| 135A | 2-carboxy-phenyl | 3-methyl isothiazol-4,5-diyl | 5 | —O— | H | 2-methyl-phenyl |
| 136A | 2-carboxy-phenyl | 3-methyl isothiazol-4,5-diyl | 5 | —S(O)₂— | H | 2-methyl-phenyl |
| 137A | 2-carboxy-phenyl | 3-methyl isothiazol-4,5-diyl | 5 | —NH— | H | 2-methyl-phenyl |
| 138A | 2-carboxy-phenyl | 3-methyl isothiazol-4,5-diyl | 5 | —O— | H | 2-chloro-phenyl |
| 139A | 2-carboxy-phenyl | 3-methyl isothiazol-4,5-diyl | 5 | —S(O)₂— | H | 2-chloro-phenyl |
| 140A | 2-carboxy-phenyl | 3-methyl isothiazol-4,5-diyl | 5 | —NH— | H | 2-chloro-phenyl |
| 141A | 2-carboxy-phenyl | 1-methyl-1,2,3-triazol-4,5-diyl | 4 | —S(O)₂— | H | phenyl |
| 142A | 2-carboxy-phenyl | 1-methyl-1,2,3-triazol-4,5-diyl | 4 | —NH— | H | phenyl |
| 143A | 2-carboxy-phenyl | 1-methyl-1,2,3-triazol-4,5-diyl | 4 | —S(O)₂— | H | 2-methyl-phenyl |
| 144A | 2-carboxy-phenyl | 1-methyl-1,2,3-triazol-4,5-diyl | 4 | —NH— | H | 2-methyl-phenyl |
| 145A | 2-carboxy-phenyl | 1-methyl-1,2,3-triazol-4,5-diyl | 4 | —S(O)₂— | H | 2-chloro-phenyl |
| 146A | 2-carboxy-phenyl | 1-methyl-1,2,3-triazol-4,5-diyl | 4 | —NH— | H | 2-chloro-phenyl |
| 147A | 2-carboxy-phenyl | 4-methyl-1,2,3-triazol-1,5-diyl | 1 | —S(O)₂— | H | phenyl |
| 148A | 2-carboxy-phenyl | 4-methyl-1,2,3-triazol-1,5-diyl | 1 | —NH— | H | phenyl |
| 149A | 2-carboxy-phenyl | 4-methyl-1,2,3-triazol-1,5-diyl | 1 | —S(O)₂— | H | 2-methyl-phenyl |
| 150A | 2-carboxy-phenyl | 4-methyl-1,2,3-triazol-1,5-diyl | 1 | —NH— | H | 2-methyl-phenyl |
| 151A | 2-carboxy-phenyl | 4-methyl-1,2,3-triazol-1,5-diyl | 1 | —S(O)₂— | H | 2-chloro-phenyl |
| 152A | 2-carboxy-phenyl | 4-methyl-1,2,3-triazol-1,5-diyl | 1 | —NH— | H | 2-chloro-phenyl |
| 153A | 2-carboxy-phenyl | 1-methyl-pyrazol-4,5-diyl | 4 | —S(O)₂— | H | phenyl |
| 154A | 2-carboxy-phenyl | 1-methyl-pyrazol-4,5-diyl | 4 | —NH— | H | phenyl |
| 155A | 2-carboxy-phenyl | 1-methyl-pyrazol-4,5-diyl | 4 | —S(O)₂— | H | 2-chloro-phenyl |
| 156A | 2-carboxy-phenyl | 1-methyl-pyrazol-4,5-diyl | 4 | —NH— | H | 2-chloro-phenyl |
| 157A | 4-carboxy-pyridin-3-yl | 5-methyl isothiazol-3,4-diyl | 3 | —O— | H | 2-methyl-phenyl |
| 158A | 2-carboxy-pyridin-3-yl | 5-methyl isothiazol-3,4-diyl | 3 | —O— | H | 2-methyl-phenyl |
| 159A | 3-carboxy-pyridin-4-yl | 5-methyl isothiazol-3,4-diyl | 3 | —O— | H | 2-methyl-phenyl |
| 160A | 3-carboxy-pyridin-2-yl | 5-methyl isothiazol-3,4-diyl | 3 | —O— | H | 2-methyl-phenyl |
| 161A | 4-carboxy-pyridin-3-yl | 3-methyl isooxazol-4,5-diyl | 5 | —O— | methyl | phenyl |
| 162A | 2-carboxy-phenyl | isooxazol-4,5-diyl | 5 | —O— | H | 2-methyl-phenyl |
| 163A | 2-carboxy-phenyl | isooxazol-4,5-diyl | 5 | —S(O)₂— | H | 2-methyl-phenyl |
| 164A | 2-carboxy-phenyl | isooxazol-4,5-diyl | 5 | —NH— | H | 2-methyl-phenyl |
| 165A | 2-carboxy-phenyl | isooxazol-4,5-diyl | 5 | —O— | H | 2-chloro-phenyl |
| 166A | 2-carboxy-phenyl | isooxazol-4,5-diyl | 5 | —S(O)₂— | H | 2-chloro-phenyl |
| 167A | 2-carboxy-phenyl | isooxazol-4,5-diyl | 5 | —NH— | H | 2-chloro-phenyl |
| 168A | 3-carboxy-pyridin-4-yl | isooxazol-3,4-diyl | 3 | —O— | H | phenyl |
| 169A | 3-carboxy-pyridin-2-yl | isooxazol-3,4-diyl | 3 | —O— | H | phenyl |
| 170A | 4-carboxy-pyridin-3-yl | isooxazol-3,4-diyl | 3 | —O— | H | phenyl |
| 171A | 2-carboxy-pyridin-3-yl | isooxazol-3,4-diyl | 3 | —O— | H | phenyl |
| 172A | 3-carboxy-pyridin-4-yl | isooxazol-3,4-diyl | 3 | —O— | H | 2-methyl-phenyl |
| 173A | 3-carboxy-pyridin-2-yl | isooxazol-3,4-diyl | 3 | —O— | H | 2-methyl-phenyl |
| 174A | 4-carboxy-pyridin-3-yl | isooxazol-3,4-diyl | 3 | —O— | H | 2-methyl-phenyl |
| 175A | 2-carboxy-pyridin-3-yl | isooxazol-3,4-diyl | 3 | —O— | H | 2-methyl-phenyl |
| 176A | 4-carboxy-pyridin-3-yl | isooxazol-3,4-diyl | 3 | —O— | H | 2-chloro-phenyl |
| 177A | 3-carboxy-pyridin-4-yl | isooxazol-3,4-diyl | 3 | —O— | H | 2-chloro-phenyl |
| 178A | 3-carboxy-pyridin-2-yl | isooxazol-3,4-diyl | 3 | —O— | H | 2-chloro-phenyl |
| 179A | 3-carboxy-pyridin-2-yl | isothiazol-3,4-diyl | 3 | —O— | H | 2-chloro-phenyl |
| 180A | 2-carboxy-pyridin-3-yl | isooxazol-3,4-diyl | 3 | —O— | H | 2-chloro-phenyl |
| 181A | 4-carboxy-pyridin-3-yl | isothiazol-3,4-diyl | 3 | —O— | H | 2-chloro-phenyl |
| 182A | 2-carboxy-pyridin-3-yl | isothiazol-3,4-diyl | 3 | —O— | H | 2-chloro-phenyl |
| 183A | 3-carboxy-pyridin-4-yl | isothiazol-3,4-diyl | 3 | —O— | H | 2-chloro-phenyl |
| 184A | 4-carboxy-pyridin-3-yl | isothiazol-3,4-diyl | 3 | —O— | H | phenyl |
| 185A | 3-carboxy-pyridin-4-yl | isothiazol-3,4-diyl | 3 | —O— | H | phenyl |
| 186A | 3-carboxy-pyridin-2-yl | isothiazol-3,4-diyl | 3 | —O— | H | phenyl |
| 187A | 2-carboxy-pyridin-3-yl | isothiazol-3,4-diyl | 3 | —O— | H | phenyl |
| 188A | 4-carboxy-pyridin-3-yl | isothiazol-4,5-diyl | 5 | —O— | H | phenyl |
| 189A | 2-carboxy-pyridin-3-yl | isothiazol-4,5-diyl | 5 | —O— | H | phenyl |
| 190A | 3-carboxy-pyridin-4-yl | isothiazol-4,5-diyl | 5 | —O— | H | phenyl |
| 191A | 3-carboxy-pyridin-2-yl | isothiazol-4,5-diyl | 5 | —O— | H | phenyl |
| 192A | 4-carboxy-pyridin-3-yl | isothiazol-4,5-diyl | 5 | —O— | H | 2-methyl-phenyl |
| 193A | 2-carboxy-pyridin-3-yl | isothiazol-4,5-diyl | 5 | —O— | H | 2-methyl-phenyl |
| 194A | 3-carboxy-pyridin-4-yl | isothiazol-4,5-diyl | 5 | —O— | H | 2-methyl-phenyl |
| 195A | 3-carboxy-pyridin-2-yl | isothiazol-4,5-diyl | 5 | —O— | H | 2-methyl-phenyl |
| 196A | 4-carboxy-pyridin-3-yl | isothiazol-4,5-diyl | 5 | —O— | H | 2-chloro-phenyl |
| 197A | 2-carboxy-pyridin-3-yl | isothiazol-4,5-diyl | 5 | —O— | H | 2-chloro-phenyl |
| 198A | 3-carboxy-pyridin-4-yl | isothiazol-4,5-diyl | 5 | —O— | H | 2-chloro-phenyl |
| 199A | 3-carboxy-pyridin-2-yl | isothiazol-4,5-diyl | 5 | —O— | H | 2-chloro-phenyl |
| 200A | 4-carboxy-pyridin-3-yl | 1,2,3-triazol-4,5-diyl | 5 | —O— | H | phenyl |
| 201A | 2-carboxy-pyridin-3-yl | 1,2,3-triazol-4,5-diyl | 5 | —O— | H | phenyl |
| 202A | 3-carboxy-pyridin-4-yl | 1,2,3-triazol-4,5-diyl | 5 | —O— | H | phenyl |
| 203A | 3-carboxy-pyridin-2-yl | 1,2,3-triazol-4,5-diyl | 5 | —O— | H | phenyl |
| 204A | 2-carboxy-pyridin-3-yl | 1,2,3-triazol-4,5-diyl | 5 | —O— | H | 2-methyl-phenyl |
| 205A | 4-carboxy-pyridin-3-yl | 1,2,3-triazol-4,5-diyl | 5 | —O— | H | 2-methyl-phenyl |
| 206A | 3-carboxy-pyridin-4-yl | 1,2,3-triazol-4,5-diyl | 5 | —O— | H | 2-methyl-phenyl |
| 207A | 3-carboxy-pyridin-2-yl | 1,2,3-triazol-4,5-diyl | 5 | —O— | H | 2-methyl-phenyl |
| 208A | 4-carboxy-pyridin-3-yl | 1,2,3-triazol-4,5-diyl | 5 | —O— | H | 2-chloro-phenyl |
| 209A | 2-carboxy-pyridin-3-yl | 1,2,3-triazol-4,5-diyl | 5 | —O— | H | 2-chloro-phenyl |
| 210A | 3-carboxy-pyridin-4-yl | 1,2,3-triazol-4,5-diyl | 5 | —O— | H | 2-chloro-phenyl |
| 211A | 3-carboxy-pyridin-2-yl | 1,2,3-triazol-4,5-diyl | 5 | —O— | H | 2-chloro-phenyl |
| 212A | 3-carboxy-pyridin-4-yl | 1,2,3-triazol-1,5-diyl | 1 | —O— | H | phenyl |
| 213A | 3-carboxy-pyridin-2-yl | 1,2,3-triazol-1,5-diyl | 1 | —O— | H | phenyl |
| 214A | 4-carboxy-pyridin-3-yl | 1,2,3-triazol-1,5-diyl | 1 | —O— | H | phenyl |
| 215A | 2-carboxy-pyridin-3-yl | 1,2,3-triazol-1,5-diyl | 1 | —O— | H | phenyl |
| 216A | 3-carboxy-pyridin-4-yl | 1,2,3-triazol-1,5-diyl | 1 | —O— | H | 2-methyl-phenyl |
| 217A | 3-carboxy-pyridin-2-yl | 1,2,3-triazol-1,5-diyl | 1 | —O— | H | 2-methyl-phenyl |
| 218A | 4-carboxy-pyridin-3-yl | 1,2,3-triazol-1,5-diyl | 1 | —O— | H | 2-methyl-phenyl |
| 219A | 2-carboxy-pyridin-3-yl | 1,2,3-triazol-1,5-diyl | 1 | —O— | H | 2-methyl-phenyl |
| 220A | 3-carboxy-pyridin-4-yl | 1,2,3-triazol-1,5-diyl | 1 | —O— | H | 2-chloro-phenyl |
| 221A | 4-carboxy-pyridin-3-yl | 1,2,3-triazol-1,5-diyl | 1 | —O— | H | 2-chloro-phenyl |
| 222A | 2-carboxy-pyridin-3-yl | 1,2,3-triazol-1,5-diyl | 1 | —O— | H | 2-chloro-phenyl |
| 223A | 3-carboxy-pyridin-2-yl | 1,2,3-triazol-1,5-diyl | 1 | —O— | H | 2-chloro-phenyl |
| 224A | 4-carboxy-pyridin-3-yl | 1H-pyrazol-4,5-diyl | 4 | —O— | H | phenyl |
| 225A | 2-carboxy-pyridin-3-yl | 1H-pyrazol-4,5-diyl | 4 | —O— | H | phenyl |
| 226A | 3-carboxy-pyridin-4-yl | 1H-pyrazol-4,5-diyl | 4 | —O— | H | phenyl |
| 227A | 3-carboxy-pyridin-2-yl | 1H-pyrazol-4,5-diyl | 4 | —O— | H | phenyl |
| 228A | 2-carboxy-phenyl | 1-methyl-pyrazol-4,5-diyl | 4 | —S(O)₂— | H | 2-methyl-phenyl |
| 229A | 2-carboxy-phenyl | 1-methyl-pyrazol-4,5-diyl | 4 | —NH— | H | 2-methyl-phenyl |
| 230A | 4-carboxy-pyridin-3-yl | 1H-pyrazol-4,5-diyl | 4 | —O— | H | 2-methyl-phenyl |
| 231A | 2-carboxy-pyridin-3-yl | 1H-pyrazol-4,5-diyl | 4 | —O— | H | 2-methyl-phenyl |
| 232A | 3-carboxy-pyridin-4-yl | 1H-pyrazol-4,5-diyl | 4 | —O— | H | 2-methyl-phenyl |
| 233A | 3-carboxy-pyridin-2-yl | 1H-pyrazol-4,5-diyl | 4 | —O— | H | 2-methyl-phenyl |
| 234A | 4-carboxy-pyridin-3-yl | 1H-pyrazol-4,5-diyl | 4 | —O— | H | 2-chloro-phenyl |
| 235A | 2-carboxy-pyridin-3-yl | 1H-pyrazol-4,5-diyl | 4 | —O— | H | 2-chloro-phenyl |
| 236A | 3-carboxy-pyridin-4-yl | 1H-pyrazol-4,5-diyl | 4 | —O— | H | 2-chloro-phenyl |
| 237A | 3-carboxy-pyridin-2-yl | 1H-pyrazol-4,5-diyl | 4 | —O— | H | 2-chloro-phenyl |
| 238A | 4-carboxy-pyridin-3-yl | 3-methyl isooxazol-4,5-diyl | 5 | —O— | H | 2-methyl-phenyl |
| 239A | 2-carboxy-pyridin-3-yl | 3-methyl isooxazol-4,5-diyl | 5 | —O— | H | 2-methyl-phenyl |
| 240A | 3-carboxy-pyridin-4-yl | 3-methyl isooxazol-4,5-diyl | 5 | —O— | H | 2-methyl-phenyl |
| 241A | 3-carboxy-pyridin-2-yl | 3-methyl isooxazol-4,5-diyl | 5 | —O— | H | 2-methyl-phenyl |
| 242A | 4-carboxy-pyridin-3-yl | 3-methyl isooxazol-4,5-diyl | 5 | —O— | H | 2-chloro-phenyl |
| 243A | 2-carboxy-pyridin-3-yl | 3-methyl isooxazol-4,5-diyl | 5 | —O— | H | 2-chloro-phenyl |
| 244A | 3-carboxy-pyridin-4-yl | 3-methyl isooxazol-4,5-diyl | 5 | —O— | H | 2-chloro-phenyl |
| 245A | 3-carboxy-pyridin-2-yl | 3-methyl isooxazol-4,5-diyl | 5 | —O— | H | 2-chloro-phenyl |
| 246A | 2-carboxy-phenyl | isooxazol-3,4-diyl | 3 | —O— | methyl | 2-methyl-phenyl |
| 247A | 2-carboxy-phenyl | isooxazol-3,4-diyl | 3 | —S(O)₂— | methyl | 2-methyl-phenyl |
| 248A | 2-carboxy-phenyl | isooxazol-3,4-diyl | 3 | —NH— | methyl | 2-methyl-phenyl |
| 249A | 2-carboxy-phenyl | 1,2,3-triazol-1,5-diyl | 1 | —S(O)₂— | methyl | 2-methyl-phenyl |
| 250A | 2-carboxy-phenyl | 1,2,3-triazol-1,5-diyl | 1 | —NH— | methyl | 2-methyl-phenyl |
| 251A | 2-carboxy-pyridin-3-yl | 3-methyl isooxazol-4,5-diyl | 5 | —O— | H | phenyl |
| 252A | 4-carboxy-pyridin-3-yl | isothiazol-4,5-diyl | 5 | —O— | methyl | phenyl |
| 253A | 2-carboxy-phenyl | 1H-pyrazol-4,5-diyl | 4 | —S— | methyl | phenyl |
| 254A | 2-carboxy-pyridin-3-yl | 3-methyl isooxazol-4,5-diyl | 5 | —O— | methyl | 2-methyl-phenyl |
| 255A | 2-carboxy-pyridin-3-yl | 3-methyl isooxazol-4,5-diyl | 5 | —O— | methyl | 2-chloro-phenyl |
| 256A | 3-carboxy-pyridin-4-yl | 3-methyl isooxazol-4,5-diyl | 5 | —O— | H | phenyl |
| 257A | 2-carboxy-pyridin-3-yl | isooxazol-4,5-diyl | 5 | —O— | methyl | phenyl |
| 258A | 4-carboxy-pyridin-3-yl | 3-methyl isothiazol-4,5-diyl | 5 | —O— | H | 2-methyl-phenyl |
| 259A | 2-carboxy-phenyl | 1-methyl-pyrazol-4,5-diyl | 4 | —S— | H | phenyl |
| 260A | 2-carboxy-pyridin-3-yl | isothiazol-3,4-diyl | 3 | —O— | H | 2-methyl-phenyl |
| 261A | 3-carboxy-pyridin-2-yl | 3-methyl isooxazol-4,5-diyl | 5 | —NH— | methyl | phenyl |
| 262A | 2-carboxy-phenyl | 3-methyl isooxazol-4,5-diyl | 5 | —S— | methyl | phenyl |
| 263A | 2-carboxy-pyridin-3-yl | 1H-pyrazol-4,5-diyl | 4 | —S— | H | phenyl |
| 264A | 3-carboxy-pyridin-4-yl | isooxazol-4,5-diyl | 5 | —O— | H | 2-methyl-phenyl |
| 265A | 3-carboxy-pyridin-2-yl | isooxazol-3,4-diyl | 3 | —NH— | H | phenyl |
| 266A | 2-carboxy-phenyl | isooxazol-3,4-diyl | 3 | —S— | H | phenyl |
| 267A | 3-carboxy-pyridin-2-yl | isooxazol-3,4-diyl | 3 | —NH— | H | 2-methyl-phenyl |
| 268A | 2-carboxy-phenyl | isooxazol-3,4-diyl | 3 | —S— | H | 2-methyl-phenyl |
| 269A | 3-carboxy-pyridin-2-yl | isooxazol-3,4-diyl | 3 | —NH— | H | 2-chloro-phenyl |
| 270A | 2-carboxy-phenyl | isooxazol-3,4-diyl | 3 | —S— | H | 2-chloro-phenyl |
| 271A | 2-carboxy-phenyl | isothiazol-3,4-diyl | 3 | —S— | H | phenyl |
| 272A | 3-carboxy-pyridin-2-yl | isothiazol-3,4-diyl | 3 | —NH— | H | 2-methyl-phenyl |
| 273A | 3-carboxy-pyridin-2-yl | isothiazol-3,4-diyl | 3 | —NH— | H | 2-chloro-phenyl |
| 274A | 3-carboxy-pyridin-2-yl | isothiazol-4,5-diyl | 5 | —NH— | H | phenyl |
| 275A | 3-carboxy-pyridin-2-yl | isothiazol-4,5-diyl | 5 | —NH— | H | 2-methyl-phenyl |
| 276A | 3-carboxy-pyridin-2-yl | isothiazol-4,5-diyl | 5 | —NH— | H | 2-chloro-phenyl |
| 277A | 3-carboxy-pyridin-2-yl | 1,2,3-triazol-4,5-diyl | 5 | —NH— | H | phenyl |
| 278A | 3-carboxy-pyridin-2-yl | 1,2,3-triazol-4,5-diyl | 5 | —NH— | H | 2-methyl-phenyl |
| 279A | 3-carboxy-pyridin-2-yl | 1,2,3-triazol-4,5-diyl | 5 | —NH— | H | 2-chloro-phenyl |
| 280A | 3-carboxy-pyridin-2-yl | 1,2,3-triazol-1,5-diyl | 1 | —NH— | H | phenyl |
| 281A | 3-carboxy-pyridin-2-yl | 1,2,3-triazol-1,5-diyl | 1 | —NH— | H | 2-methyl-phenyl |
| 282A | 3-carboxy-pyridin-2-yl | 1,2,3-triazol-1,5-diyl | 1 | —NH— | H | 2-chloro-phenyl |
| 283A | 3-carboxy-pyridin-2-yl | 1H-pyrazol-4,5-diyl | 4 | —NH— | H | phenyl |
| 284A | 3-carboxy-pyridin-2-yl | 1H-pyrazol-4,5-diyl | 4 | —NH— | H | 2-chloro-phenyl |
| 285A | 3-carboxy-pyridin-2-yl | 3-methyl isooxazol-4,5-diyl | 5 | —NH— | H | 2-methyl-phenyl |
| 286A | 3-carboxy-pyridin-2-yl | 3-methyl isooxazol-4,5-diyl | 5 | —NH— | H | 2-chloro-phenyl |
| 287A | 3-carboxy-pyridin-2-yl | isothiazol-3,4-diyl | 3 | —O— | H | 2-methyl-phenyl |
| 288A | 3-carboxy-pyridin-2-yl | 1H-pyrazol-4,5-diyl | 4 | —S— | H | phenyl |
| 289A | 3-carboxy-pyridin-4-yl | isothiazol-3,4-diyl | 3 | —O— | H | 2-methyl-phenyl |
| 290A | 2-carboxy-phenyl | isothiazol-3,4-diyl | 3 | —S— | H | 2-methyl-phenyl |
| 291A | 4-carboxy-pyridin-3-yl | isothiazol-3,4-diyl | 3 | —O— | H | 2-methyl-phenyl |
| 292A | 2-carboxy-phenyl | isothiazol-3,4-diyl | 3 | —S— | H | 2-chloro-phenyl |
| 293A | 2-carboxy-phenyl | isothiazol-4,5-diyl | 5 | —S— | H | phenyl |
| 294A | 2-carboxy-phenyl | isothiazol-4,5-diyl | 5 | —S— | H | 2-methyl-phenyl |
| 295A | 4-carboxy-pyridin-3-yl | 1H-pyrazol-4,5-diyl | 4 | —S— | H | phenyl |
| 296A | 2-carboxy-phenyl | isothiazol-4,5-diyl | 5 | —S— | H | 2-chloro-phenyl |
| 297A | 2-carboxy-phenyl | 1,2,3-triazol-4,5-diyl | 5 | —S— | H | phenyl |
| 298A | 2-carboxy-phenyl | 1,2,3-triazol-4,5-diyl | 5 | —S— | H | 2-methyl-phenyl |
| 299A | 2-carboxy-phenyl | 1,2,3-triazol-4,5-diyl | 5 | —S— | H | 2-chloro-phenyl |
| 300A | 2-carboxy-phenyl | 1,2,3-triazol-1,5-diyl | 1 | —S— | H | phenyl |
| 301A | 2-carboxy-phenyl | 1,2,3-triazol-1,5-diyl | 1 | —S— | H | 2-methyl-phenyl |
| 302A | 2-carboxy-phenyl | 1,2,3-triazol-1,5-diyl | 1 | —S— | H | 2-chloro-phenyl |
| 303A | 2-carboxy-phenyl | 1H-pyrazol-4,5-diyl | 4 | —S— | H | 2-methyl-phenyl |
| 304A | 2-carboxy-phenyl | 1H-pyrazol-4,5-diyl | 4 | —S— | H | 2-chloro-phenyl |
| 305A | 2-carboxy-phenyl | 3-methyl isooxazol-4,5-diyl | 5 | —S— | H | 2-methyl-phenyl |
| 306A | 2-carboxy-phenyl | 3-methyl isooxazol-4,5-diyl | 5 | —S— | H | 2-chloro-phenyl |
| 307A | 3-carboxy-pyridin-4-yl | 1H-pyrazol-4,5-diyl | 4 | —S— | H | phenyl |
| 308A | 6-carboxy- | isooxazol-3,4-diyl | 3 | —O— | methyl | phenyl |
| | benzo[d]thiazol-5 -yl | | | | | |
| 309A | 6-carboxy- | isooxazol-3,4-diyl | 3 | —S(O)₂— | methyl | phenyl |
| | benzo[d]thiazol-5 -yl | | | | | |
| 310A | 6-carboxy- | isooxazol-3,4-diyl | 3 | —NH— | methyl | phenyl |
| | benzo[d]thiazol-5 -yl | | | | | |
| 311A | 4-carboxy- | 1,2,3-triazol-4,5-diyl | 5 | —S(O)₂— | methyl | phenyl |
| | benzo[d]thiazol-5 -yl | | | | | |
| 312A | 4-carboxy- | 1,2,3-triazol-4,5-diyl | 5 | —NH— | methyl | phenyl |
| | benzo[d]thiazol-5 -yl | | | | | |
| 313A | 5-carboxy- | isooxazol-3,4-diyl | 3 | —O— | methyl | phenyl |
| | benzo[d]oxazol-4-yl | | | | | |
| 314A | 5-carboxy- | isooxazol-3,4-diyl | 3 | —S(O)₂— | methyl | phenyl |
| | benzo[d]oxazol-4-yl | | | | | |
| 315A | 5-carboxy- | isooxazol-3,4-diyl | 3 | —NH— | methyl | phenyl |
| | benzo[d]oxazol-4-yl | | | | | |
| 316A | 5-carboxy- | isooxazol-3,4-diyl | 3 | —O— | methyl | phenyl |
| | benzo[d]thiazol-4-yl | | | | | |
| 317A | 5-carboxy- | isooxazol-3,4-diyl | 3 | —S(O)₂— | methyl | phenyl |
| | benzo[d]thiazol-4-yl | | | | | |
| 318A | 5-carboxy- | isooxazol-3,4-diyl | 3 | —NH— | methyl | phenyl |
| | benzo[d]thiazol-4-yl | | | | | |
| 319A | 6-carboxy- | isooxazol-3,4-diyl | 3 | —O— | methyl | phenyl |
| | benzo[d]oxazol-5-yl | | | | | |
| 320A | 6-carboxy- | isooxazol-3,4-diyl | 3 | —S(O)₂— | methyl | phenyl |
| | benzo[d]oxazol-5-yl | | | | | |
| 321A | 6-carboxy- | isooxazol-3,4-diyl | 3 | —NH— | methyl | phenyl |
| | benzo[d]oxazol-5-yl | | | | | |
| 322A | 4-carboxy- | 1,2,3-triazol-4,5-diyl | 5 | —S(O)₂— | methyl | phenyl |
| | benzo[d]oxazol-5-yl | | | | | |
| 323A | 4-carboxy- | 1,2,3-triazol-4,5-diyl | 5 | —NH— | methyl | phenyl |
| | benzo[d]oxazol-5-yl | | | | | |
| 324A | 6-carboxy- | 5-methyl isooxazol-3,4-diyl | 3 | —O— | H | phenyl |
| | benzo[d]thiazol-5 -yl | | | | | |
| 325A | 6-carboxy- | 5-methyl isooxazol-3,4-diyl | 3 | —S(O)₂— | H | phenyl |
| | benzo[d]thiazol-5 -yl | | | | | |
| 326A | 6-carboxy- | 5-methyl isooxazol-3,4-diyl | 3 | —NH— | H | phenyl |
| | benzo[d]thiazol-5 -yl | | | | | |
| 327A | 4-carboxy- | 1-methyl-1,2,3-triazol-4,5-diyl | 4 | —S(O)₂— | H | 2-methyl-phenyl |
| | benzo[d]thiazol-5-yl | | | | | |
| 328A | 4-carboxy- | 1-methyl-1,2,3-triazol-4,5-diyl | 4 | —NH— | H | 2-methyl-phenyl |
| | benzo[d]thiazol-5-yl | | | | | |
| 329A | 5-carboxy- | 5-methyl isooxazol-3,4-diyl | 3 | —O— | H | phenyl |
| | benzo[d]oxazol-4-yl | | | | | |
| 330A | 5-carboxy- | 5-methyl isooxazol-3,4-diyl | 3 | —S(O)₂— | H | phenyl |
| | benzo[d]oxazol-4-yl | | | | | |
| 331A | 5-carboxy- | 5-methyl isooxazol-3,4-diyl | 3 | —NH— | H | phenyl |
| | benzo[d]oxazol-4-yl | | | | | |
| 332A | 5-carboxy- | 5-methyl isooxazol-3,4-diyl | 3 | —O— | H | phenyl |
| | benzo[d]thiazol-4-yl | | | | | |
| 333A | 5-carboxy- | 5-methyl isooxazol-3,4-diyl | 3 | —S(O)₂— | H | phenyl |
| | benzo[d]thiazol-4-yl | | | | | |
| 334A | 5-carboxy- | 5-methyl isooxazol-3,4-diyl | 3 | —NH— | H | phenyl |
| | benzo[d]thiazol-4-yl | | | | | |
| 335A | 6-carboxy- | 5-methyl isooxazol-3,4-diyl | 3 | —O— | H | phenyl |
| | benzo[d]oxazol-5-yl | | | | | |
| 336A | 6-carboxy- | 5-methyl isooxazol-3,4-diyl | 3 | —S(O)₂— | H | phenyl |
| | benzo[d]oxazol-5-yl | | | | | |
| 337A | 6-carboxy- | 5-methyl isooxazol-3,4-diyl | 3 | —NH— | H | phenyl |
| | benzo[d]oxazol-5-yl | | | | | |
| 338A | 4-carboxy- | 1-methyl-1,2,3-triazol-4,5-diyl | 4 | —S(O)₂— | H | 2-methyl-phenyl |
| | benzo[d]oxazol-5-yl | | | | | |
| 339A | 4-carboxy- | 1-methyl-1,2,3-triazol-4,5diyl- | 4 | —NH— | H | 2-methyl-phenyl |
| | benzo[d]oxazol-5-yl | | | | | |
| 340A | 4-carboxy- | 3-methyl isooxazol-4,5-diyl | 5 | —O— | methyl | phenyl |
| | benzo[d]thiazol-5 -yl | | | | | |
| 341A | 4-carboxy- | 3-methyl isooxazol-4,5-diyl | 5 | —S(O)₂— | methyl | phenyl |
| | benzo[d]thiazol-5 -yl | | | | | |
| 342A | 4-carboxy- | 3-methyl isooxazol-4,5-diyl | 5 | —NH— | methyl | phenyl |
| | benzo[d]thiazol-5 -yl | | | | | |
| 343A | 6-carboxy- | 3-methyl isooxazol-4,5-diyl | 5 | —O— | methyl | phenyl |
| | benzo[d]thiazol-5 -yl | | | | | |
| 344A | 6-carboxy- | 3-methyl isooxazol-4,5-diyl | 5 | —S(O)₂— | methyl | phenyl |
| | benzo[d]thiazol-5 -yl | | | | | |
| 345A | 6-carboxy- | 3-methyl isooxazol-4,5-diyl | 5 | —NH— | methyl | phenyl |
| | benzo[d]thiazol-5 -yl | | | | | |
| 346A | 2-carboxy-pyridin-3-yl | 3-methyl isooxazol-4,5-diyl | 5 | —S(O)₂— | H | 2-chloro-phenyl |
| 347A | 5-carboxy- | 3-methyl isooxazol-4,5-diyl | 5 | —O— | methyl | phenyl |
| | benzo[d]oxazol-4-yl | | | | | |
| 348A | 5-carboxy- | 3-methyl isooxazol-4,5-diyl | 5 | —S(O)₂— | methyl | phenyl |
| | benzo[d]oxazol-4-yl | | | | | |
| 349A | 5-carboxy- | 3-methyl isooxazol-4,5-diyl | 5 | —NH— | methyl | phenyl |
| | benzo[d]oxazol-4-yl | | | | | |
| 350A | 5-carboxy- | 3-methyl isooxazol-4,5-diyl | 5 | —O— | methyl | phenyl |
| | benzo[d]thiazol-4-yl | | | | | |
| 351A | 5-carboxy- | 3-methyl isooxazol-4,5-diyl | 5 | —S(O)₂— | methyl | phenyl |
| | benzo[d]thiazol-4-yl | | | | | |
| 352A | 5-carboxy- | 3-methyl isooxazol-4,5-diyl | 5 | —NH— | methyl | phenyl |
| | benzo[d]thiazol-4-yl | | | | | |
| 353A | 6-carboxy- | 3-methyl isooxazol-4,5-diyl | 5 | —O— | methyl | phenyl |
| | benzo[d]oxazol-5-yl | | | | | |
| 354A | 6-carboxy- | 3-methyl isooxazol-4,5-diyl | 5 | —S(O)₂— | methyl | phenyl |
| | benzo[d]oxazol-5-yl | | | | | |
| 355A | 6-carboxy- | 3-methyl isooxazol-4,5-diyl | 5 | —NH— | methyl | phenyl |
| | benzo[d]oxazol-5-yl | | | | | |
| 356A | 4-carboxy- | 3-methyl isooxazol-4,5-diyl | 5 | —O— | methyl | phenyl |
| | benzo[d]oxazol-5-yl | | | | | |
| 357A | 4-carboxy- | 3-methyl isooxazol-4,5-diyl | 5 | —S(O)₂— | methyl | phenyl |
| | benzo[d]oxazol-5-yl | | | | | |
| 358A | 4-carboxy- | 3-methyl isooxazol-4,5-diyl | 5 | —NH— | methyl | phenyl |
| | benzo[d]oxazol-5-yl | | | | | |
| 359A | 6-carboxy- | 1H-pyrazol-4,5-diyl | 4 | —S(O)₂— | H | 2-methyl-phenyl |
| | benzo[d]thiazol-5 -yl | | | | | |
| 360A | 6-carboxy- | 1H-pyrazol-4,5-diyl | 4 | —NH— | H | 2-methyl-phenyl |
| | benzo[d]thiazol-5 -yl | | | | | |
| 361A | 6-carboxy- | 1,2,3-triazol-4,5-diyl | 5 | —S(O)₂— | H | phenyl |
| | benzo[d]thiazol-5 -yl | | | | | |
| 362A | 4-carboxy- | 1,2,3-triazol-4,5-diyl | 5 | —NH— | H | phenyl |
| | benzo[d]thiazol-5 -yl | | | | | |
| 363A | 6-carboxy- | isothiazol-3,4-diyl | 3 | —O— | H | phenyl |
| | benzo[d]thiazol-5 -yl | | | | | |
| 364A | 6-carboxy- | isothiazol-3,4-diyl | 3 | —S(O)₂— | H | phenyl |
| | benzo[d]thiazol-5 -yl | | | | | |
| 365A | 6-carboxy- | isothiazol-3,4-diyl | 3 | —NH— | H | phenyl |
| | benzo[d]thiazol-5 -yl | | | | | |
| 366A | 6-carboxy- | isothiazol-4,5-diyl | 5 | —O— | H | 2-methyl-phenyl |
| | benzo[d]thiazol-5 -yl | | | | | |
| 367A | 6-carboxy- | isothiazol-4,5-diyl | 5 | —S(O)₂— | H | 2-methyl-phenyl |
| | benzo[d]thiazol-5 -yl | | | | | |
| 368A | 6-carboxy- | isothiazol-4,5-diyl | 5 | —NH— | H | 2-methyl-phenyl |
| | benzo[d]thiazol-5 -yl | | | | | |
| 369A | 4-carboxy- | isooxazol-3,4-diyl | 3 | —O— | H | phenyl |
| | benzo[d]thiazol-5 -yl | | | | | |
| 370A | 4-carboxy- | isooxazol-3,4-diyl | 3 | —S(O)₂— | H | phenyl |
| | benzo[d]thiazol-5 -yl | | | | | |
| 371A | 4-carboxy- | isooxazol-3,4-diyl | 3 | —NH— | H | phenyl |
| | benzo[d]thiazol-5 -yl | | | | | |
| 372A | 6-carboxy- | 1,2,3-triazol-1,5-diyl | 1 | —S(O)₂— | H | 2-methyl-phenyl |
| | benzo[d]thiazol-5 -yl | | | | | |
| 373A | 6-carboxy- | 1,2,3-triazol-1,5-diyl | 1 | —NH— | H | 2-methyl-phenyl |
| | benzo[d]thiazol-5 -yl | | | | | |
| 374A | 6-carboxy- | 1H-pyrazol-4,5-diyl | 4 | —S(O)₂— | H | 2-chloro-phenyl |
| | benzo[d]thiazol-5 -yl | | | | | |
| 375A | 6-carboxy- | 1H-pyrazol-4,5-diyl | 4 | —NH— | H | 2-chloro-phenyl |
| | benzo[d]thiazol-5 -yl | | | | | |
| 376A | 6-carboxy- | isooxazol-3,4-diyl | 3 | —O— | H | 2-methyl-phenyl |
| | benzo[d]thiazol-5 -yl | | | | | |
| 377A | 6-carboxy- | isooxazol-3,4-diyl | 3 | —S(O)₂— | H | 2-methyl-phenyl |
| | benzo[d]thiazol-5 -yl | | | | | |
| 378A | 6-carboxy- | isooxazol-3,4-diyl | 3 | —NH— | H | 2-methyl-phenyl |
| | benzo[d]thiazol-5 -yl | | | | | |
| 379A | 6-carboxy- | isothiazol-3,4-diyl | 3 | —O— | H | 2-chloro-phenyl |
| | benzo[d]thiazol-5 -yl | | | | | |
| 380A | 6-carboxy- | isothiazol-3,4-diyl | 3 | —S(O)₂— | H | 2-chloro-phenyl |
| | benzo[d]thiazol-5 -yl | | | | | |
| 381A | 6-carboxy- | isothiazol-3,4-diyl | 3 | —NH— | H | 2-chloro-phenyl |
| | benzo[d]thiazol-5 -yl | | | | | |
| 382A | 6-carboxy- | isothiazol-4,5-diyl | 5 | —O— | H | 2-chloro-phenyl |
| | benzo[d]thiazol-5 -yl | | | | | |
| 383A | 6-carboxy- | isothiazol-4,5-diyl | 5 | —S(O)₂— | H | 2-chloro-phenyl |
| | benzo[d]thiazol-5 -yl | | | | | |
| 384A | 6-carboxy- | isothiazol-4,5-diyl | 5 | —NH— | H | 2-chloro-phenyl |
| | benzo[d]thiazol-5 -yl | | | | | |
| 385A | 6-carboxy- | 1,2,3-triazol-4,5-diyl | 5 | —S(O)₂— | H | phenyl |
| | benzo[d]thiazol-5 -yl | | | | | |
| 386A | 6-carboxy- | 1,2,3-triazol-4,5-diyl | 5 | —NH— | H | phenyl |
| | benzo[d]thiazol-5 -yl | | | | | |
| 387A | 6-carboxy- | 1,2,3-triazol-1,5-diyl | 1 | —S(O)₂— | H | phenyl |
| | benzo[d]thiazol-5 -yl | | | | | |
| 388A | 6-carboxy- | 1,2,3-triazol-1,5-diyl | 1 | —NH— | H | phenyl |
| | benzo[d]thiazol-5 -yl | | | | | |
| 389A | 6-carboxy- | 1H-pyrazol-4,5-diyl | 4 | —S(O)₂— | H | phenyl |
| | benzo[d]thiazol-5 -yl | | | | | |
| 390A | 6-carboxy- | 1H-pyrazol-4,5-diyl | 4 | —NH— | H | phenyl |
| | benzo[d]thiazol-5 -yl | | | | | |
| 391A | 6-carboxy- | 3-methyl isooxazol-4,5-diyl | 5 | —O— | H | 2-chloro-phenyl |
| | benzo[d]thiazol-5 -yl | | | | | |
| 392A | 6-carboxy- | 3-methyl isooxazol-4,5-diyl | 5 | —S(O)₂— | H | 2-chloro-phenyl |
| | benzo[d]thiazol-5 -yl | | | | | |
| 393A | 6-carboxy- | 3-methyl isooxazol-4,5-diyl | 5 | —NH— | H | 2-chloro-phenyl |
| | benzo[d]thiazol-5 -yl | | | | | |
| 394A | 6-carboxy- | isooxazol-3,4-diyl | 3 | —O— | H | 2-chloro-phenyl |
| | benzo[d]thiazol-5 -yl | | | | | |
| 395A | 6-carboxy- | isooxazol-3,4-diyl | 3 | —S(O)₂— | H | 2-chloro-phenyl |
| | benzo[d]thiazol-5 -yl | | | | | |
| 396A | 6-carboxy- | isooxazol-3,4-diyl | 3 | —NH— | H | 2-chloro-phenyl |
| | benzo[d]thiazol-5 -yl | | | | | |
| 397A | 6-carboxy- | isothiazol-3,4-diyl | 3 | —O— | H | 2-methyl-phenyl |
| | benzo[d]thiazol-5 -yl | | | | | |
| 398A | 6-carboxy- | isothiazol-3,4-diyl | 3 | —S(O)₂— | H | 2-methyl-phenyl |
| | benzo[d]thiazol-5 -yl | | | | | |
| 399A | 6-carboxy- | isothiazol-3,4-diyl | 3 | —NH— | H | 2-methyl-phenyl |
| | benzo[d]thiazol-5 -yl | | | | | |
| 400A | 6-carboxy- | isothiazol-4,5-diyl | 5 | —O— | H | phenyl |
| | benzo[d]thiazol-5 -yl | | | | | |
| 401A | 6-carboxy- | isothiazol-4,5-diyl | 5 | —S(O)₂— | H | phenyl |
| | benzo[d]thiazol-5 -yl | | | | | |
| 402A | 6-carboxy- | isothiazol-4,5-diyl | 5 | —NH— | H | phenyl |
| | benzo[d]thiazol-5 -yl | | | | | |
| 403A | 6-carboxy- | 1,2,3-triazol-4,5-diyl | 5 | —S(O)₂— | H | 2-methyl-phenyl |
| | benzo[d]thiazol-5 -yl | | | | | |
| 404A | 6-carboxy- | 1,2,3-triazol-4,5-diyl | 5 | —NH— | H | 2-methyl-phenyl |
| | benzo[d]thiazol-5 -yl | | | | | |
| 405A | 6-carboxy- | 1,2,3-triazol-4,5-diyl | 5 | —S(O)₂— | H | 2-chloro-phenyl |
| | benzo[d]thiazol-5 -yl | | | | | |
| 406A | 6-carboxy- | 1,2,3-triazol-4,5-diyl | 5 | —NH— | H | 2-chloro-phenyl |
| | benzo[d]thiazol-5 -yl | | | | | |
| 407A | 6-carboxy- | 1,2,3-triazol-1,5-diyl | 1 | —S(O)₂— | H | 2-chloro-phenyl |
| | benzo[d]thiazol-5 -yl | | | | | |
| 408A | 6-carboxy- | 1,2,3-triazol-1,5-diyl | 1 | —NH— | H | 2-chloro-phenyl |
| | benzo[d]thiazol-5 -yl | | | | | |
| 409A | 6-carboxy- | 1H-pyrazol-4,5-diyl | 4 | —S— | H | phenyl |
| | benzo[d]thiazol-5 -yl | | | | | |
| 410A | 6-carboxy- | 3-methyl isooxazol-4,5-diyl | 5 | —O— | H | 2-methyl-phenyl |
| | benzo[d]thiazol-5 -yl | | | | | |
| 411A | 6-carboxy- | 3-methyl isooxazol-4,5-diyl | 5 | —S(O)₂— | H | 2-methyl-phenyl |
| | benzo[d]thiazol-5 -yl | | | | | |
| 412A | 6-carboxy- | 3-methyl isooxazol-4,5-diyl | 5 | —NH— | H | 2-methyl-phenyl |
| | benzo[d]thiazol-5 -yl | | | | | |
| 413A | 4-carboxy- | isooxazol-3,4-diyl | 3 | —O— | H | phenyl |
| | benzo[d]oxazol-5-yl | | | | | |
| 414A | 4-carboxy- | isooxazol-3,4-diyl | 3 | —S(O)₂— | H | phenyl |
| | benzo[d]oxazol-5-yl | | | | | |
| 415A | 4-carboxy- | isooxazol-3,4-diyl | 3 | —NH— | H | phenyl |
| | benzo[d]oxazol-5-yl | | | | | |
| 416A | 4-carboxy- | isooxazol-3,4-diyl | 3 | —O— | H | 2-methyl-phenyl |
| | benzo[d]oxazol-5-yl | | | | | |
| 417A | 4-carboxy- | isooxazol-3,4-diyl | 3 | —S(O)₂— | H | 2-methyl-phenyl |
| | benzo[d]oxazol-5-yl | | | | | |
| 418A | 4-carboxy- | isooxazol-3,4-diyl | 3 | —NH— | H | 2-methyl-phenyl |
| | benzo[d]oxazol-5-yl | | | | | |
| 419A | 5-carboxy- | isooxazol-3,4-diyl | 3 | —O— | H | 2-methyl-phenyl |
| | benzo[d]oxazol-4-yl | | | | | |
| 420A | 5-carboxy- | isooxazol-3,4-diyl | 3 | —S(O)₂— | H | 2-methyl-phenyl |
| | benzo[d]oxazol-4-yl | | | | | |
| 421A | 5-carboxy- | isooxazol-3,4-diyl | 3 | —NH— | H | 2-methyl-phenyl |
| | benzo[d]oxazol-4-yl | | | | | |
| 422A | 4-carboxy- | isooxazol-3,4-diyl | 3 | —O— | H | 2-methyl-phenyl |
| | benzo[d]thiazol-5 -yl | | | | | |
| 423A | 4-carboxy- | isooxazol-3,4-diyl | 3 | —S(O)₂— | H | 2-methyl-phenyl |
| | benzo[d]thiazol-5 -yl | | | | | |
| 424A | 4-carboxy- | isooxazol-3,4-diyl | 3 | —NH— | H | 2-methyl-phenyl |
| | benzo[d]thiazol-5 -yl | | | | | |
| 425A | 5-carboxy- | isooxazol-3,4-diyl | 3 | —O— | H | 2-methyl-phenyl |
| | benzo[d]thiazol-4-yl | | | | | |
| 426A | 5-carboxy- | isooxazol-3,4-diyl | 3 | —S(O)₂— | H | 2-methyl-phenyl |
| | benzo[d]thiazol-4-yl | | | | | |
| 427A | 5-carboxy- | isooxazol-3,4-diyl | 3 | —NH— | H | 2-methyl-phenyl |
| | benzo[d]thiazol-4-yl | | | | | |
| 428A | 6-carboxy- | isooxazol-3,4-diyl | 3 | —O— | H | 2-methyl-phenyl |
| | benzo[d]oxazol-5-yl | | | | | |
| 429A | 6-carboxy- | isooxazol-3,4-diyl | 3 | —S(O)₂— | H | 2-methyl-phenyl |
| | benzo[d]oxazol-5-yl | | | | | |
| 430A | 6-carboxy- | isooxazol-3,4-diyl | 3 | —NH— | H | 2-methyl-phenyl |
| | benzo[d]oxazol-5-yl | | | | | |
| 431A | 4-carboxy- | isooxazol-3,4-diyl | 3 | —O— | H | 2-chloro-phenyl |
| | benzo[d]oxazol-5-yl | | | | | |
| 432A | 4-carboxy- | isooxazol-3,4-diyl | 3 | —S(O)₂— | H | 2-chloro-phenyl |
| | benzo[d]oxazol-5-yl | | | | | |
| 433A | 4-carboxy- | isooxazol-3,4-diyl | 3 | —NH— | H | 2-chloro-phenyl |
| | benzo[d]oxazol-5-yl | | | | | |
| 434A | 4-carboxy- | 1,2,3-triazol-4,5-diyl | 5 | —S(O)₂— | H | 2-chloro-phenyl |
| | benzo[d]oxazol-5-yl | | | | | |
| 435A | 4-carboxy- | 1,2,3-triazol-4,5-diyl | 5 | —NH— | H | 2-chloro-phenyl |
| | benzo[d]oxazol-5-yl | | | | | |
| 436A | 5-carboxy- | isooxazol-3,4-diyl | 3 | —O— | H | 2-chloro-phenyl |
| | benzo[d]thiazol-4-yl | | | | | |
| 437A | 5-carboxy- | isooxazol-3,4-diyl | 3 | —S(O)₂— | H | 2-chloro-phenyl |
| | benzo[d]thiazol-4-yl | | | | | |
| 438A | 5-carboxy- | isooxazol-3,4-diyl | 3 | —NH— | H | 2-chloro-phenyl |
| | benzo[d]thiazol-4-yl | | | | | |
| 439A | 4-carboxy- | isooxazol-3,4-diyl | 3 | —O— | H | 2-chloro-phenyl |
| | benzo[d]thiazol-5 -yl | | | | | |
| 440A | 4-carboxy- | isooxazol-3,4-diyl | 3 | —S(O)₂— | H | 2-chloro-phenyl |
| | benzo[d]thiazol-5 -yl | | | | | |
| 441A | 4-carboxy- | isooxazol-3,4-diyl | 3 | —NH— | H | 2-chloro-phenyl |
| | benzo[d]thiazol-5 -yl | | | | | |
| 442A | 5-carboxy- | isooxazol-3,4-diyl | 3 | —O— | H | 2-chloro-phenyl |
| | benzo[d]oxazol-4-yl | | | | | |
| 443A | 5-carboxy- | isooxazol-3,4-diyl | 3 | —S(O)₂— | H | 2-chloro-phenyl |
| | benzo[d]oxazol-4-yl | | | | | |
| 444A | 5-carboxy- | isooxazol-3,4-diyl | 3 | —NH— | H | 2-chloro-phenyl |
| | benzo[d]oxazol-4-yl | | | | | |
| 445A | 6-carboxy- | isooxazol-3,4-diyl | 3 | —O— | H | 2-chloro-phenyl |
| | benzo[d]oxazol-5-yl | | | | | |
| 446A | 6-carboxy- | isooxazol-3,4-diyl | 3 | —S(O)₂— | H | 2-chloro-phenyl |
| | benzo[d]oxazol-5-yl | | | | | |
| 447A | 6-carboxy- | isooxazol-3,4-diyl | 3 | —NH— | H | 2-chloro-phenyl |
| | benzo[d]oxazol-5-yl | | | | | |
| 448A | 4-carboxy- | isothiazol-3,4-diyl | 3 | —O— | H | phenyl |
| | benzo[d]thiazol-5 -yl | | | | | |
| 449A | 4-carboxy- | isothiazol-3,4-diyl | 3 | —S(O)₂— | H | phenyl |
| | benzo[d]thiazol-5 -yl | | | | | |
| 450A | 4-carboxy- | isothiazol-3,4-diyl | 3 | —NH— | H | phenyl |
| | benzo[d]thiazol-5 -yl | | | | | |
| 451A | 6-carboxy- | isothiazol-3,4-diyl | 3 | —O— | H | phenyl |
| | benzo[d]oxazol-5-yl | | | | | |
| 452A | 6-carboxy- | isothiazol-3,4-diyl | 3 | —S(O)₂— | H | phenyl |
| | benzo[d]oxazol-5-yl | | | | | |
| 453A | 6-carboxy- | isothiazol-3,4-diyl | 3 | —NH— | H | phenyl |
| | benzo[d]oxazol-5-yl | | | | | |
| 454A | 4-carboxy- | isothiazol-3,4-diyl | 3 | —O— | H | phenyl |
| | benzo[d]oxazol-5-yl | | | | | |
| 455A | 4-carboxy- | isothiazol-3,4-diyl | 3 | —S(O)₂— | H | phenyl |
| | benzo[d]oxazol-5-yl | | | | | |
| 456A | 4-carboxy- | isothiazol-3,4-diyl | 3 | —NH— | H | phenyl |
| | benzo[d]oxazol-5-yl | | | | | |
| 457A | 5-carboxy- | isothiazol-3,4-diyl | 3 | —O— | H | phenyl |
| | benzo[d]oxazol-4-yl | | | | | |
| 458A | 5-carboxy- | isothiazol-3,4-diyl | 3 | —S(O)₂— | H | phenyl |
| | benzo[d]oxazol-4-yl | | | | | |
| 459A | 5-carboxy- | isothiazol-3,4-diyl | 3 | —NH— | H | phenyl |
| | benzo[d]oxazol-4-yl | | | | | |
| 460A | 5-carboxy- | isothiazol-3,4-diyl | 3 | —O— | H | phenyl |
| | benzo[d]thiazol-4 -yl | | | | | |
| 461A | 5-carboxy- | isothiazol-3,4-diyl | 3 | —S(O)₂— | H | phenyl |
| | benzo[d]thiazol-4 -yl | | | | | |
| 462A | 5-carboxy- | isothiazol-3,4-diyl | 3 | —NH— | H | phenyl |
| | benzo[d]thiazol-4 -yl | | | | | |
| 463A | 3-carboxy-pyridin-2-yl | 3-methyl isooxazol-4,5-diyl | 5 | —S(O)₂— | H | 2-chloro-phenyl |
| 464A | 4-carboxy- | isothiazol-3,4-diyl | 3 | —O— | H | 2-methyl-phenyl |
| | benzo[d]oxazol-5-yl | | | | | |
| 465A | 4-carboxy- | isothiazol-3,4-diyl | 3 | —S(O)₂— | H | 2-methyl-phenyl |
| | benzo[d]oxazol-5-yl | | | | | |
| 466A | 4-carboxy- | isothiazol-3,4-diyl | 3 | —NH— | H | 2-methyl-phenyl |
| | benzo[d]oxazol-5-yl | | | | | |
| 467A | 5-carboxy- | isothiazol-3,4-diyl | 3 | —O— | H | 2-methyl-phenyl |
| | benzo[d]oxazol-4-yl | | | | | |
| 468A | 5-carboxy- | isothiazol-3,4-diyl | 3 | —S(O)₂— | H | 2-methyl-phenyl |
| | benzo[d]oxazol-4-yl | | | | | |
| 469A | 5-carboxy- | isothiazol-3,4-diyl | 3 | —NH— | H | 2-methyl-phenyl |
| | benzo[d]oxazol-4-yl | | | | | |
| 470A | 6-carboxy- | isothiazol-3,4-diyl | 3 | —O— | H | 2-methyl-phenyl |
| | benzo[d]oxazol-5-yl | | | | | |
| 471A | 6-carboxy- | isothiazol-3,4-diyl | 3 | —S(O)₂— | H | 2-methyl-phenyl |
| | benzo[d]oxazol-5-yl | | | | | |
| 472A | 6-carboxy- | isothiazol-3,4-diyl | 3 | —NH— | H | 2-methyl-phenyl |
| | benzo[d]oxazol-5-yl | | | | | |
| 473A | 4-carboxy- | isothiazol-3,4-diyl | 3 | —O— | H | 2-methyl-phenyl |
| | benzo[d]thiazol-5 -yl | | | | | |
| 474A | 4-carboxy- | isothiazol-3,4-diyl | 3 | —S(O)₂— | H | 2-methyl-phenyl |
| | benzo[d]thiazol-5 -yl | | | | | |
| 475A | 4-carboxy- | isothiazol-3,4-diyl | 3 | —NH— | H | 2-methyl-phenyl |
| | benzo[d]thiazol-5 -yl | | | | | |
| 476A | 5-carboxy- | isothiazol-3,4-diyl | 3 | —O— | H | 2-methyl-phenyl |
| | benzo[d]thiazol-4 -yl | | | | | |
| 477A | 5-carboxy- | isothiazol-3,4-diyl | 3 | —S(O)₂— | H | 2-methyl-phenyl |
| | benzo[d]thiazol-4-yl | | | | | |
| 478A | 5-carboxy- | isothiazol-3,4-diyl | 3 | —NH— | H | 2-methyl-phenyl |
| | benzo[d]thiazol-4-yl | | | | | |
| 479A | 6-carboxy- | isothiazol-3,4-diyl | 3 | —O— | H | 2-chloro-phenyl |
| | benzo[d]oxazol-5-yl | | | | | |
| 480A | 6-carboxy- | isothiazol-3,4-diyl | 3 | —S(O)₂— | H | 2-chloro-phenyl |
| | benzo[d]oxazol-5-yl | | | | | |
| 481A | 6-carboxy- | isothiazol-3,4-diyl | 3 | —NH— | H | 2-chloro-phenyl |
| | benzo[d]oxazol-5-yl | | | | | |
| 482A | 5-carboxy- | isothiazol-3,4-diyl | 3 | —O— | H | 2-chloro-phenyl |
| | benzo[d]oxazol-4-yl | | | | | |
| 483A | 5-carboxy- | isothiazol-3,4-diyl | 3 | —S(O)₂— | H | 2-chloro-phenyl |
| | benzo[d]oxazol-4-yl | | | | | |
| 484A | 5-carboxy- | isothiazol-3,4-diyl | 3 | —NH— | H | 2-chloro-phenyl |
| | benzo[d]oxazol-4-yl | | | | | |
| 485A | 5-carboxy- | isothiazol-3,4-diyl | 3 | —O— | H | 2-chloro-phenyl |
| | benzo[d]thiazol-4-yl | | | | | |
| 486A | 5-carboxy- | isothiazol-3,4-diyl | 3 | —S(O)₂— | H | 2-chloro-phenyl |
| | benzo[d]thiazol-4-yl | | | | | |
| 487A | 5-carboxy- | isothiazol-3,4-diyl | 3 | —NH— | H | 2-chloro-phenyl |
| | benzo[d]thiazol-4-yl | | | | | |
| 488A | 4-carboxy- | isothiazol-3,4-diyl | 3 | —O— | H | 2-chloro-phenyl |
| | benzo[d]thiazol-5 -yl | | | | | |
| 489A | 4-carboxy- | isothiazol-3,4-diyl | 3 | —S(O)₂— | H | 2-chloro-phenyl |
| | benzo[d]thiazol-5 -yl | | | | | |
| 490A | 4-carboxy- | isothiazol-3,4-diyl | 3 | —NH— | H | 2-chloro-phenyl |
| | benzo[d]thiazol-5-yl | | | | | |
| 491A | 4-carboxy- | isothiazol-3,4-diyl | 3 | —O— | H | 2-chloro-phenyl |
| | benzo[d]oxazol-5-yl | | | | | |
| 492A | 4-carboxy- | isothiazol-3,4-diyl | 3 | —S(O)₂— | H | 2-chloro-phenyl |
| | benzo[d]oxazol-5-yl | | | | | |
| 493A | 4-carboxy- | isothiazol-3,4-diyl | 3 | —NH— | H | 2-chloro-phenyl |
| | benzo[d]oxazol-5-yl | | | | | |
| 494A | 5-carboxy- | isothiazol-4,5-diyl | 5 | —O— | H | phenyl |
| | benzo[d]thiazol-4-yl | | | | | |
| 495A | 5-carboxy- | isothiazol-4,5-diyl | 5 | —S(O)₂— | H | phenyl |
| | benzo[d]thiazol-4-yl | | | | | |
| 496A | 5-carboxy- | isothiazol-4,5-diyl | 5 | —NH— | H | phenyl |
| | benzo[d]thiazol-4-yl | | | | | |
| 497A | 5-carboxy- | isothiazol-4,5-diyl | 5 | —O— | H | phenyl |
| | benzo[d]oxazol-4-yl | | | | | |
| 498A | 5-carboxy- | isothiazol-4,5-diyl | 5 | —S(O)₂— | H | phenyl |
| | benzo[d]oxazol-4-yl | | | | | |
| 499A | 5-carboxy- | isothiazol-4,5-diyl | 5 | —NH— | H | phenyl |
| | benzo[d]oxazol-4-yl | | | | | |
| 500A | 6-carboxy- | isothiazol-4,5-diyl | 5 | —O— | H | phenyl |
| | benzo[d]oxazol-5-yl | | | | | |
| 501A | 6-carboxy- | isothiazol-4,5-diyl | 5 | —S(O)₂— | H | phenyl |
| | benzo[d]oxazol-5-yl | | | | | |
| 502A | 6-carboxy- | isothiazol-4,5-diyl | 5 | —NH— | H | phenyl |
| | benzo[d]oxazol-5-yl | | | | | |
| 503A | 4-carboxy- | isothiazol-4,5-diyl | 5 | —O— | H | phenyl |
| | benzo[d]oxazol-5-yl | | | | | |
| 504A | 4-carboxy- | isothiazol-4,5-diyl | 5 | —S(O)₂— | H | phenyl |
| | benzo[d]oxazol-5-yl | | | | | |
| 505A | 4-carboxy- | isothiazol-4,5-diyl | 5 | —NH— | H | phenyl |
| | benzo[d]oxazol-5-yl | | | | | |
| 506A | 4-carboxy- | isothiazol-4,5-diyl | 5 | —O— | H | phenyl |
| | benzo[d]thiazol-5 -yl | | | | | |
| 507A | 4-carboxy- | isothiazol-4,5-diyl | 5 | —S(O)₂— | H | phenyl |
| | benzo[d]thiazol-5 -yl | | | | | |
| 508A | 4-carboxy- | isothiazol-4,5-diyl | 5 | —NH— | H | phenyl |
| | benzo[d]thiazol-5 -yl | | | | | |
| 509A | 4-carboxy- | isothiazol-4,5-diyl | 5 | —O— | H | 2-methyl-phenyl |
| | benzo[d]oxazol-5-yl | | | | | |
| 510A | 4-carboxy- | isothiazol-4,5-diyl | 5 | —S(O)₂— | H | 2-methyl-phenyl |
| | benzo[d]oxazol-5-yl | | | | | |
| 511A | 4-carboxy- | isothiazol-4,5-diyl | 5 | —NH— | H | 2-methyl-phenyl |
| | benzo[d]oxazol-5-yl | | | | | |
| 512A | 4-carboxy- | isothiazol-4,5-diyl | 5 | —O— | H | 2-methyl-phenyl |
| | benzo[d]thiazol-5 -yl | | | | | |
| 513A | 4-carboxy- | isothiazol-4,5-diyl | 5 | —S(O)₂— | H | 2-methyl-phenyl |
| | benzo[d]thiazol-5 -yl | | | | | |
| 514A | 4-carboxy- | isothiazol-4,5-diyl | 5 | —NH— | H | 2-methyl-phenyl |
| | benzo[d]thiazol-5 -yl | | | | | |
| 515A | 5-carboxy- | isothiazol-4,5-diyl | 5 | —O— | H | 2-methyl-phenyl |
| | benzo[d]oxazol-4-yl | | | | | |
| 516A | 5-carboxy- | isothiazol-4,5-diyl | 5 | —S(O)₂— | H | 2-methyl-phenyl |
| | benzo[d]oxazol-4-yl | | | | | |
| 517A | 5-carboxy- | isothiazol-4,5-diyl | 5 | —NH— | H | 2-methyl-phenyl |
| | benzo[d]oxazol-4-yl | | | | | |
| 518A | 6-carboxy- | isothiazol-4,5-diyl | 5 | —O— | H | 2-methyl-phenyl |
| | benzo[d]oxazol-5-yl | | | | | |
| 519A | 6-carboxy- | isothiazol-4,5-diyl | 5 | —S(O)₂— | H | 2-methyl-phenyl |
| | benzo[d]oxazol-5-yl | | | | | |
| 520A | 6-carboxy- | isothiazol-4,5-diyl | 5 | —NH— | H | 2-methyl-phenyl |
| | benzo[d]oxazol-5-yl | | | | | |
| 521A | 4-carboxy-pyridin-3-yl | 3-methyl isooxazol-4,5-diyl | 5 | —S(O)₂— | H | 2-chloro-phenyl |
| 522A | 5-carboxy- | isothiazol-4,5-diyl | 5 | —O— | H | 2-methyl-phenyl |
| | benzo[d]thiazol-4-yl | | | | | |
| 523A | 5-carboxy- | isothiazol-4,5-diyl | 5 | —S(O)₂— | H | 2-methyl-phenyl |
| | benzo[d]thiazol-4-yl | | | | | |
| 524A | 5-carboxy- | isothiazol-4,5-diyl | 5 | —NH— | H | 2-methyl-phenyl |
| | benzo[d]thiazol-4-yl | | | | | |
| 525A | 4-carboxy- | isothiazol-4,5-diyl | 5 | —O— | H | 2-chloro-phenyl |
| | benzo[d]oxazol-5-yl | | | | | |
| 526A | 4-carboxy- | isothiazol-4,5-diyl | 5 | —S(O)₂— | H | 2-chloro-phenyl |
| | benzo[d]oxazol-5-yl | | | | | |
| 527A | 4-carboxy- | isothiazol-4,5-diyl | 5 | —NH— | H | 2-chloro-phenyl |
| | benzo[d]oxazol-5-yl | | | | | |
| 528A | 5-carboxy- | isothiazol-4,5-diyl | 5 | —O— | H | 2-chloro-phenyl |
| | benzo[d]oxazol-4-yl | | | | | |
| 529A | 5-carboxy- | isothiazol-4,5-diyl | 5 | —S(O)₂— | H | 2-chloro-phenyl |
| | benzo[d]oxazol-4-yl | | | | | |
| 530A | 5-carboxy- | isothiazol-4,5-diyl | 5 | —NH— | H | 2-chloro-phenyl |
| | benzo[d]oxazol-4-yl | | | | | |
| 531A | 6-carboxy- | isothiazol-4,5-diyl | 5 | —O— | H | 2-chloro-phenyl |
| | benzo[d]oxazol-5-yl | | | | | |
| 532A | 6-carboxy- | isothiazol-4,5-diyl | 5 | —S(O)₂— | H | 2-chloro-phenyl |
| | benzo[d]oxazol-5-yl | | | | | |
| 533A | 6-carboxy- | isothiazol-4,5-diyl | 5 | —NH— | H | 2-chloro-phenyl |
| | benzo[d]oxazol-5-yl | | | | | |
| 534A | 5-carboxy- | isothiazol-4,5-diyl | 5 | —O— | H | 2-chloro-phenyl |
| | benzo[d]thiazol-4-yl | | | | | |
| 535A | 5-carboxy- | isothiazol-4,5-diyl | 5 | —S(O)₂— | H | 2-chloro-phenyl |
| | benzo[d]thiazol-4-yl | | | | | |
| 536A | 5-carboxy- | isothiazol-4,5-diyl | 5 | —NH— | H | 2-chloro-phenyl |
| | benzo[d]thiazol-4-yl | | | | | |
| 537A | 4-carboxy- | isothiazol-4,5-diyl | 5 | —O— | H | 2-chloro-phenyl |
| | benzo[d]thiazol-5 -yl | | | | | |
| 538A | 4-carboxy- | isothiazol-4,5-diyl | 5 | —S(O)₂— | H | 2-chloro-phenyl |
| | benzo[d]thiazol-5 -yl | | | | | |
| 539A | 4-carboxy- | isothiazol-4,5-diyl | 5 | —NH— | H | 2-chloro-phenyl |
| | benzo[d]thiazol-5 -yl | | | | | |
| 540A | 6-carboxy- | 1,2,3-triazol-4,5-diyl | 5 | —S(O)₂— | H | phenyl |
| | benzo[d]oxazol-5-yl | | | | | |
| 541A | 6-carboxy- | 1,2,3-triazol-4,5-diyl | 5 | —NH— | H | phenyl |
| | benzo[d]oxazol-5-yl | | | | | |
| 542A | 5-carboxy- | 1,2,3-triazol-4,5-diyl | 5 | —S(O)₂— | H | phenyl |
| | benzo[d]oxazol-4-yl | | | | | |
| 543A | 5-carboxy- | 1,2,3-triazol-4,5-diyl | 5 | —NH— | H | phenyl |
| | benzo[d]oxazol-4-yl | | | | | |
| 544A | 5-carboxy- | 1,2,3-triazol-4,5-diyl | 5 | —S(O)₂— | H | phenyl |
| | benzo[d]thiazol-4-yl | | | | | |
| 545A | 5-carboxy- | 1,2,3-triazol-4,5-diyl | 5 | —NH— | H | phenyl |
| | benzo[d]thiazol-4-yl | | | | | |
| 546A | 4-carboxy- | 1,2,3-triazol-4,5-diyl | 5 | —S(O)₂— | H | phenyl |
| | benzo[d]oxazol-5-yl | | | | | |
| 547A | 4-carboxy- | 1,2,3-triazol-4,5-diyl | 5 | —NH— | H | phenyl |
| | benzo[d]oxazol-5-yl | | | | | |
| 548A | 4-carboxy- | 1H-pyrazol-4,5-diyl | 4 | —S(O)₂— | H | phenyl |
| | benzo[d]thiazol-5 -yl | | | | | |
| 549A | 4-carboxy- | 1H-pyrazol-4,5-diyl | 4 | —NH— | H | phenyl |
| | benzo[d]thiazol-5 -yl | | | | | |
| 550A | 4-carboxy- | 1H-pyrazol-4,5-diyl | 4 | —S(O)₂— | H | 2-methyl-phenyl |
| | benzo[d]thiazol-5 -yl | | | | | |
| 551A | 4-carboxy- | 1H-pyrazol-4,5-diyl | 4 | —NH— | H | 2-methyl-phenyl |
| | benzo[d]thiazol-5 -yl | | | | | |
| 552A | 4-carboxy- | 3-methyl isooxazol-4,5-diyl | 5 | —O— | H | 2-methyl-phenyl |
| | benzo[d]thiazol-5 -yl | | | | | |
| 553A | 4-carboxy- | 3-methyl isooxazol-4,5-diyl | 5 | —S(O)₂— | H | 2-methyl-phenyl |
| | benzo[d]thiazol-5 -yl | | | | | |
| 554A | 4-carboxy- | 3-methyl isooxazol-4,5-diyl | 5 | —NH— | H | 2-methyl-phenyl |
| | benzo[d]thiazol-5 -yl | | | | | |
| 555A | 4-carboxy- | 1,2,3-triazol-1,5-diyl | 1 | —S(O)₂— | H | 2-chloro-phenyl |
| | benzo[d]thiazol-5 -yl | | | | | |
| 556A | 4-carboxy- | 1,2,3-triazol-1,5-diyl | 1 | —NH— | H | 2-chloro-phenyl |
| | benzo[d]thiazol-5 -yl | | | | | |
| 557A | 4-carboxy- | 1H-pyrazol-4,5-diyl | 4 | —S(O)₂— | H | 2-chloro-phenyl |
| | benzo[d]thiazol-5 -yl | | | | | |
| 558A | 4-carboxy- | 1H-pyrazol-4,5-diyl | 4 | —NH— | H | 2-chloro-phenyl |
| | benzo[d]thiazol-5 -yl | | | | | |
| 559A | 4-carboxy- | 3-methyl isooxazol-4,5-diyl | 5 | —O— | H | 2-chloro-phenyl |
| | benzo[d]thiazol-5 -yl | | | | | |
| 560A | 4-carboxy- | 3-methyl isooxazol-4,5-diyl | 5 | —S(O)₂— | H | 2-chloro-phenyl |
| | benzo[d]thiazol-5 -yl | | | | | |
| 561A | 4-carboxy- | 3-methyl isooxazol-4,5-diyl | 5 | —NH— | H | 2-chloro-phenyl |
| | benzo[d]thiazol-5 -yl | | | | | |
| 562A | 5-carboxy- | 1,2,3-triazol-4,5-diyl | 5 | —S(O)₂— | H | 2-methyl-phenyl |
| | benzo[d]oxazol-4-yl | | | | | |
| 563A | 5-carboxy- | 1,2,3-triazol-4,5-diyl | 5 | —NH— | H | 2-methyl-phenyl |
| | benzo[d]oxazol-4-yl | | | | | |
| 564A | 6-carboxy- | 1,2,3-triazol-4,5-diyl | 5 | —S(O)₂— | H | 2-methyl-phenyl |
| | benzo[d]oxazol-5-yl | | | | | |
| 565A | 6-carboxy- | 1,2,3-triazol-4,5-diyl | 5 | —NH— | H | 2-methyl-phenyl |
| | benzo[d]oxazol-5-yl | | | | | |
| 566A | 4-carboxy- | 1,2,3-triazol-1,5-diyl | 1 | —S(O)₂— | H | 2-methyl-phenyl |
| | benzo[d]thiazol-5 -yl | | | | | |
| 567A | 4-carboxy- | 1,2,3-triazol-1,5-diyl | 1 | —NH— | H | 2-methyl-phenyl |
| | benzo[d]thiazol-5 -yl | | | | | |
| 568A | 4-carboxy- | 1,2,3-triazol-4,5-diyl | 5 | —S(O)₂— | H | 2-chloro-phenyl |
| | benzo[d]thiazol-5 -yl | | | | | |
| 569A | 4-carboxy- | 1,2,3-triazol-4,5-diyl | 5 | —NH— | H | 2-chloro-phenyl |
| | benzo[d]thiazol-5 -yl | | | | | |
| 570A | 4-carboxy- | 1,2,3-triazol-1,5-diyl | 1 | —S(O)₂— | H | phenyl |
| | benzo[d]thiazol-5 -yl | | | | | |
| 571A | 4-carboxy- | 1,2,3-triazol-1,5-diyl | 1 | —NH— | H | phenyl |
| | benzo[d]thiazol-5 -yl | | | | | |
| 572A | 4-carboxy- | 1H-pyrazol-4,5-diyl | 4 | —S— | H | phenyl |
| | benzo[d]thiazol-5 -yl | | | | | |
| 573A | 5-carboxy- | 1,2,3-triazol-4,5-diyl | 5 | —S(O)₂— | H | 2-methyl-phenyl |
| | benzo[d]thiazol-4-yl | | | | | |
| 574A | 5-carboxy- | 1,2,3-triazol-4,5-diyl | 5 | —NH— | H | 2-methyl-phenyl |
| | benzo[d]thiazol-4-yl | | | | | |
| 575A | 6-carboxy- | 1,2,3-triazol-4,5-diyl | 5 | —S(O)₂— | H | 2-chloro-phenyl |
| | benzo[d]oxazol-5-yl | | | | | |
| 576A | 6-carboxy- | 1,2,3-triazol-4,5-diyl | 5 | —NH— | H | 2-chloro-phenyl |
| | benzo[d]oxazol-5-yl | | | | | |
| 577A | 5-carboxy- | 1,2,3-triazol-4,5-diyl | 5 | —S(O)₂— | H | 2-chloro-phenyl |
| | benzo[d]oxazol-4-yl | | | | | |
| 578A | 5-carboxy- | 1,2,3-triazol-4,5-diyl | 5 | —NH— | H | 2-chloro-phenyl |
| | benzo[d]oxazol-4-yl | | | | | |
| 579A | 5-carboxy- | 1,2,3-triazol-4,5-diyl | 5 | —S(O)₂— | H | 2-chloro-phenyl |
| | benzo[d]thiazol-4-yl | | | | | |
| 580A | 5-carboxy- | 1,2,3-triazol-4,5-diyl | 5 | —NH— | H | 2-chloro-phenyl |
| | benzo[d]thiazol-4-yl | | | | | |
| 581A | 6-carboxy- | 1,2,3-triazol-1,5-diyl | 1 | —S(O)₂— | H | phenyl |
| | benzo[d]oxazol-5-yl | | | | | |
| 582A | 6-carboxy- | 1,2,3-triazol-1,5-diyl | 1 | —NH— | H | phenyl |
| | benzo[d]oxazol-5-yl | | | | | |
| 583A | 4-carboxy- | 1,2,3-triazol-1,5-diyl | 1 | —S(O)₂— | H | phenyl |
| | benzo[d]oxazol-5-yl | | | | | |
| 584A | 4-carboxy- | 1,2,3-triazol-1,5-diyl | 1 | —NH— | H | phenyl |
| | benzo[d]oxazol-5-yl | | | | | |
| 585A | 5-carboxy- | 1,2,3-triazol-1,5-diyl | 1 | —S(O)₂— | H | phenyl |
| | benzo[d]thiazol-4-yl | | | | | |
| 586A | 5-carboxy- | 1,2,3-triazol-1,5-diyl | 1 | —NH— | H | phenyl |
| | benzo[d]thiazol-4-yl | | | | | |
| 587A | 5-carboxy- | 1,2,3-triazol-1,5-diyl | 1 | —S(O)₂— | H | phenyl |
| | benzo[d]oxazol-4-yl | | | | | |
| 588A | 5-carboxy- | 1,2,3-triazol-1,5-diyl | 1 | —NH— | H | phenyl |
| | benzo[d]oxazol-4-yl | | | | | |
| 589A | 5-carboxy- | 1,2,3-triazol-1,5-diyl | 1 | —S(O)₂— | H | 2-methyl-phenyl |
| | benzo[d]oxazol-4-yl | | | | | |
| 590A | 5-carboxy- | 1,2,3-triazol-1,5-diyl | 1 | —NH— | H | 2-methyl-phenyl |
| | benzo[d]oxazol-4-yl | | | | | |
| 591A | 6-carboxy- | 1,2,3-triazol-1,5-diyl | 1 | —S(O)₂— | H | 2-methyl-phenyl |
| | benzo[d]oxazol-5-yl | | | | | |
| 592A | 6-carboxy- | 1,2,3-triazol-1,5-diyl | 1 | —NH— | H | 2-methyl-phenyl |
| | benzo[d]oxazol-5-yl | | | | | |
| 593A | 4-carboxy- | 1,2,3-triazol-1,5-diyl | 1 | —S(O)₂— | H | 2-methyl-phenyl |
| | benzo[d]oxazol-5-yl | | | | | |
| 594A | 4-carboxy- | 1,2,3-triazol-1,5-diyl | 1 | —NH— | H | 2-methyl-phenyl |
| | benzo[d]oxazol-5-yl | | | | | |
| 595A | 5-carboxy- | 1,2,3-triazol-1,5-diyl | 1 | —S(O)₂— | H | 2-methyl-phenyl |
| | benzo[d]thiazol-4-yl | | | | | |
| 596A | 5-carboxy- | 1,2,3-triazol-1,5-diyl | 1 | —NH— | H | 2-methyl-phenyl |
| | benzo[d]thiazol-4-yl | | | | | |
| 597A | 6-carboxy- | 1,2,3-triazol-1,5-diyl | 1 | —S(O)₂— | H | 2-chloro-phenyl |
| | benzo[d]oxazol-5-yl | | | | | |
| 598A | 6-carboxy- | 1,2,3-triazol-1,5-diyl | 1 | —NH— | H | 2-chloro-phenyl |
| | benzo[d]oxazol-5-yl | | | | | |
| 599A | 5-carboxy- | 1,2,3-triazol-1,5-diyl | 1 | —S(O)₂— | H | 2-chloro-phenyl |
| | benzo[d]oxazol-4-yl | | | | | |
| 600A | 5-carboxy- | 1,2,3-triazol-1,5-diyl | 1 | —NH— | H | 2-chloro-phenyl |
| | benzo[d]oxazol-4-yl | | | | | |
| 601A | 4-carboxy- | 1,2,3-triazol-1,5-diyl | 1 | —S(O)₂— | H | 2-chloro-phenyl |
| | benzo[d]oxazol-5-yl | | | | | |
| 602A | 4-carboxy- | 1,2,3-triazol-1,5-diyl | 1 | —NH— | H | 2-chloro-phenyl |
| | benzo[d]oxazol-5-yl | | | | | |
| 603A | 5-carboxy- | 1,2,3-triazol-1,5-diyl | 1 | —S(O)₂— | H | 2-chloro-phenyl |
| | benzo[d]thiazol-4-yl | | | | | |
| 604A | 5-carboxy- | 1,2,3-triazol-1,5-diyl | 1 | —NH— | H | 2-chloro-phenyl |
| | benzo[d]thiazol-4-yl | | | | | |
| 605A | 5-carboxy- | 1H-pyrazol-4,5-diyl | 4 | —S(O)₂— | H | phenyl |
| | benzo[d]oxazol-4-yl | | | | | |
| 606A | 5-carboxy- | 1H-pyrazol-4,5-diyl | 4 | —NH— | H | phenyl |
| | benzo[d]oxazol-4-yl | | | | | |
| 607A | 5-carboxy- | 1H-pyrazol-4,5-diyl | 4 | —S(O)₂— | H | phenyl |
| | benzo[d]thiazol-4-yl | | | | | |
| 608A | 5-carboxy- | 1H-pyrazol-4,5-diyl | 4 | —NH— | H | phenyl |
| | benzo[d]thiazol-4-yl | | | | | |
| 609A | 6-carboxy- | 1H-pyrazol-4,5-diyl | 4 | —S(O)₂— | H | phenyl |
| | benzo[d]oxazol-5-yl | | | | | |
| 610A | 6-carboxy- | 1H-pyrazol-4,5-diyl | 4 | —NH— | H | phenyl |
| | benzo[d]oxazol-5-yl | | | | | |
| 611A | 4-carboxy- | 1H-pyrazol-4,5-diyl | 4 | —S(O)₂— | H | phenyl |
| | benzo[d]oxazol-5-yl | | | | | |
| 612A | 4-carboxy- | 1H-pyrazol-4,5-diyl | 4 | —NH— | H | phenyl |
| | benzo[d]oxazol-5-yl | | | | | |
| 613A | 5-carboxy- | 1H-pyrazol-4,5-diyl | 4 | —S— | H | phenyl |
| | benzo[d]oxazol-4-yl | | | | | |
| 614A | 5-carboxy- | 1H-pyrazol-4,5-diyl | 4 | —S— | H | phenyl |
| | benzo[d]thiazol-4-yl | | | | | |
| 615A | 6-carboxy- | 1H-pyrazol-4,5-diyl | 4 | —S— | H | phenyl |
| | benzo[d]oxazol-5-yl | | | | | |
| 616A | 4-carboxy- | 1H-pyrazol-4,5-diyl | 4 | —S— | H | phenyl |
| | benzo[d]oxazol-5-yl | | | | | |
| 617A | 5-carboxy- | 1H-pyrazol-4,5-diyl | 4 | —S(O)₂— | H | 2-methyl-phenyl |
| | benzo[d]oxazol-4-yl | | | | | |
| 618A | 5-carboxy- | 1H-pyrazol-4,5-diyl | 4 | —NH— | H | 2-methyl-phenyl |
| | benzo[d]oxazol-4-yl | | | | | |
| 619A | 5-carboxy- | 1H-pyrazol-4,5-diyl | 4 | —S(O)₂— | H | 2-methyl-phenyl |
| | benzo[d]thiazol-4-yl | | | | | |
| 620A | 5-carboxy- | 1H-pyrazol-4,5-diyl | 4 | —NH— | H | 2-methyl-phenyl |
| | benzo[d]thiazol-4-yl | | | | | |
| 621A | 6-carboxy- | 1H-pyrazol-4,5-diyl | 4 | —S(O)₂— | H | 2-methyl-phenyl |
| | benzo[d]oxazol-5-yl | | | | | |
| 622A | 6-carboxy- | 1H-pyrazol-4,5-diyl | 4 | —NH— | H | 2-methyl-phenyl |
| | benzo[d]oxazol-5-yl | | | | | |
| 623A | 4-carboxy- | 1H-pyrazol-4,5-diyl | 4 | —S(O)₂— | H | 2-methyl-phenyl |
| | benzo[d]oxazol-5-yl | | | | | |
| 624A | 4-carboxy- | 1H-pyrazol-4,5-diyl | 4 | —NH— | H | 2-methyl-phenyl |
| | benzo[d]oxazol-5-yl | | | | | |
| 625A | 4-carboxy- | 1H-pyrazol-4,5-diyl | 4 | —S(O)₂— | H | 2-chloro-phenyl |
| | benzo[d]oxazol-5-yl | | | | | |
| 626A | 4-carboxy- | 1H-pyrazol-4,5-diyl | 4 | —NH— | H | 2-chloro-phenyl |
| | benzo[d]oxazol-5-yl | | | | | |
| 627A | 5-carboxy- | 1H-pyrazol-4,5-diyl | 4 | —S(O)₂— | H | 2-chloro-phenyl |
| | benzo[d]oxazol-4-yl | | | | | |
| 628A | 5-carboxy- | 1H-pyrazol-4,5-diyl | 4 | —NH— | H | 2-chloro-phenyl |
| | benzo[d]oxazol-4-yl | | | | | |
| 629A | 5-carboxy- | 1H-pyrazol-4,5-diyl | 4 | —S(O)₂— | H | 2-chloro-phenyl |
| | benzo[d]thiazol-4-yl | | | | | |
| 630A | 5-carboxy- | 1H-pyrazol-4,5-diyl | 4 | —NH— | H | 2-chloro-phenyl |
| | benzo[d]thiazol-4-yl | | | | | |
| 631A | 6-carboxy- | 1H-pyrazol-4,5-diyl | 4 | —S(O)₂— | H | 2-chloro-phenyl |
| | benzo[d]oxazol-5-yl | | | | | |
| 632A | 6-carboxy- | 1H-pyrazol-4,5-diyl | 4 | —NH— | H | 2-chloro-phenyl |
| | benzo[d]oxazol-5-yl | | | | | |
| 633A | 5-carboxy- | 3-methyl isooxazol-4,5-diyl | 5 | —O— | H | 2-methyl-phenyl |
| | benzo[d]thiazol-4-yl | | | | | |
| 634A | 5-carboxy- | 3-methyl isooxazol-4,5-diyl | 5 | —S(O)₂— | H | 2-methyl-phenyl |
| | benzo[d]thiazol-4-yl | | | | | |
| 635A | 5-carboxy- | 3-methyl isooxazol-4,5-diyl | 5 | —NH— | H | 2-methyl-phenyl |
| | benzo[d]thiazol-4-yl | | | | | |
| 636A | 6-carboxy- | 3-methyl isooxazol-4,5-diyl | 5 | —O— | H | 2-methyl-phenyl |
| | benzo[d]oxazol-5-yl | | | | | |
| 637A | 6-carboxy- | 3-methyl isooxazol-4,5-diyl | 5 | —S(O)₂— | H | 2-methyl-phenyl |
| | benzo[d]oxazol-5-yl | | | | | |
| 638A | 6-carboxy- | 3-methyl isooxazol-4,5-diyl | 5 | —NH— | H | 2-methyl-phenyl |
| | benzo[d]oxazol-5-yl | | | | | |
| 639A | 4-carboxy- | 3-methyl isooxazol-4,5-diyl | 5 | —O— | H | 2-methyl-phenyl |
| | benzo[d]oxazol-5-yl | | | | | |
| 640A | 4-carboxy- | 3-methyl isooxazol-4,5-diyl | 5 | —S(O)₂— | H | 2-methyl-phenyl |
| | benzo[d]oxazol-5-yl | | | | | |
| 641A | 4-carboxy- | 3-methyl isooxazol-4,5-diyl | 5 | —NH— | H | 2-methyl-phenyl |
| | benzo[d]oxazol-5-yl | | | | | |
| 642A | 3-carboxy-pyridin-4-yl | 3-methyl isooxazol-4,5-diyl | 5 | —S(O)₂— | H | 2-chloro-phenyl |
| 643A | 5-carboxy- | 3-methyl isooxazol-4,5-diyl | 5 | —O— | H | 2-methyl-phenyl |
| | benzo[d]oxazol-4-yl | | | | | |
| 644A | 5-carboxy- | 3-methyl isooxazol-4,5-diyl | 5 | —S(O)₂— | H | 2-methyl-phenyl |
| | benzo[d]oxazol-4-yl | | | | | |
| 645A | 5-carboxy- | 3-methyl isooxazol-4,5-diyl | 5 | —NH— | H | 2-methyl-phenyl |
| | benzo[d]oxazol-4-yl | | | | | |
| 646A | 4-carboxy- | 3-methyl isooxazol-4,5-diyl | 5 | —O— | H | 2-chloro-phenyl |
| | benzo[d]oxazol-5-yl | | | | | |
| 647A | 4-carboxy- | 3-methyl isooxazol-4,5-diyl | 5 | —S(O)₂— | H | 2-chloro-phenyl |
| | benzo[d]oxazol-5-yl | | | | | |
| 648A | 4-carboxy- | 3-methyl isooxazol-4,5-diyl | 5 | —NH— | H | 2-chloro-phenyl |
| | benzo[d]oxazol-5-yl | | | | | |
| 649A | 5-carboxy- | 3-methyl isooxazol-4,5-diyl | 5 | —O— | H | 2-chloro-phenyl |
| | benzo[d]oxazol-4-yl | | | | | |
| 650A | 5-carboxy- | 3-methyl isooxazol-4,5-diyl | 5 | —S(O)₂— | H | 2-chloro-phenyl |
| | benzo[d]oxazol-4-yl | | | | | |
| 651A | 5-carboxy- | 3-methyl isooxazol-4,5-diyl | 5 | —NH— | H | 2-chloro-phenyl |
| | benzo[d]oxazol-4-yl | | | | | |
| 652A | 5-carboxy- | 3-methyl isooxazol-4,5-diyl | 5 | —O— | H | 2-chloro-phenyl |
| | benzo[d]thiazol-4-yl | | | | | |
| 653A | 5-carboxy- | 3-methyl isooxazol-4,5-diyl | 5 | —S(O)₂— | H | 2-chloro-phenyl |
| | benzo[d]thiazol-4-yl | | | | | |
| 654A | 5-carboxy- | 3-methyl isooxazol-4,5-diyl | 5 | —NH— | H | 2-chloro-phenyl |
| | benzo[d]thiazol-4-yl | | | | | |
| 655A | 6-carboxy- | 3-methyl isooxazol-4,5-diyl | 5 | —O— | H | 2-chloro-phenyl |
| | benzo[d]oxazol-5-yl | | | | | |
| 656A | 6-carboxy- | 3-methyl isooxazol-4,5-diyl | 5 | —S(O)₂— | H | 2-chloro-phenyl |
| | benzo[d]oxazol-5-yl | | | | | |
| 657A | 6-carboxy- | 3-methyl isooxazol-4,5-diyl | 5 | —NH— | H | 2-chloro-phenyl |
| | benzo[d]oxazol-5-yl | | | | | |

Note:
Column * indicates ring C's point of connection to the adjacent ring B.

In some embodiments, compounds of Formula (II) are also represented by Formula (II-B) and are selected from the following compounds as listed in Table 2B.

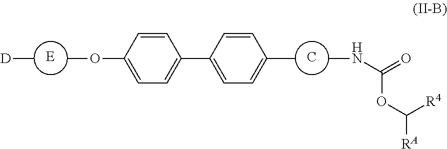

TABLE 2B

| Cmpd # II- | 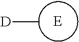 | 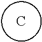 | * | R⁴ | R^{A} |
|---|---|---|---|---|---|
| 1B | 5-carboxy-thiazol-4-yl | isothiazol-3,4-diyl | 3 | methyl | phenyl |
| 2B | 5-carboxy-oxazol-4-yl | isothiazol-3,4-diyl | 3 | methyl | phenyl |
| 3B | 5-carboxy-thiazol-4-yl | 5-methyl isothiazol-3,4-diyl | 3 | H | phenyl |
| 4B | 5-carboxy-oxazol-4-yl | 5-methyl isothiazol-3,4-diyl | 3 | H | phenyl |
| 5B | 5-carboxy-thiazol-4-yl | 3-methyl isooxazol-4,5-diyl | 5 | methyl | phenyl |
| 6B | 5-carboxy-oxazol-4-yl | 3-methyl isooxazol-4,5-diyl | 5 | methyl | phenyl |
| 7B | 5-carboxy-thiazol-4-yl | isooxazol-3,4-diyl | 3 | H | phenyl |
| 8B | 5-carboxy-oxazol-4-yl | isooxazol-3,4-diyl | 3 | H | phenyl |
| 9B | 5-carboxy-thiazol-4-yl | isooxazol-3,4-diyl | 3 | H | 2-methyl-phenyl |
| 10B | 5-carboxy-oxazol-4-yl | isooxazol-3,4-diyl | 3 | H | 2-methyl-phenyl |
| 11B | 5-carboxy-thiazol-4-yl | isothiazol-3,4-diyl | 3 | H | 2-methyl-phenyl |
| 12B | 5-carboxy-oxazol-4-yl | isothiazol-3,4-diyl | 3 | H | 2-methyl-phenyl |
| 13B | 5-carboxy-thiazol-4-yl | isooxazol-3,4-diyl | 3 | H | 2-chloro-phenyl |
| 14B | 5-carboxy-oxazol-4-yl | isooxazol-3,4-diyl | 3 | H | 2-chloro-phenyl |
| 15B | 5-carboxy-oxazol-4-yl | isothiazol-3,4-diyl | 3 | H | 2-chloro-phenyl |
| 16B | 5-carboxy-thiazol-4-yl | 3-methyl isooxazol-4,5-diyl | 5 | H | 2-methyl-phenyl |
| 17B | 5-carboxy-thiazol-4-yl | 3-methyl isooxazol-4,5-diyl | 5 | H | 2-chloro-phenyl |
| 18B | 5-carboxy-thiazol-4-yl | isothiazol-3,4-diyl | 3 | H | 2-chloro-phenyl |
| 19B | 5-carboxy-thiazol-4-yl | isothiazol-4,5-diyl | 5 | H | phenyl |
| 20B | 5-carboxy-thiazol-4-yl | isothiazol-4,5-diyl | 5 | H | 2-methyl-phenyl |
| 21B | 5-carboxy-thiazol-4-yl | isothiazol-4,5-diyl | 5 | H | 2-chloro-phenyl |
| 22B | 5-carboxy-thiazol-4-yl | 1,2,3-triazol-4,5-diyl | 5 | H | phenyl |
| 23B | 5-carboxy-thiazol-4-yl | 1,2,3-triazol-4,5-diyl | 5 | H | 2-methyl-phenyl |
| 24B | 5-carboxy-thiazol-4-yl | 1,2,3-triazol-4,5-diyl | 5 | H | 2-chloro-phenyl |
| 25B | 5-carboxy-thiazol-4-yl | 1,2,3-triazol-1,5-diyl | 1 | H | phenyl |
| 26B | 5-carboxy-thiazol-4-yl | 1,2,3-triazol-1,5-diyl | 1 | H | 2-methyl-phenyl |
| 27B | 5-carboxy-thiazol-4-yl | 1,2,3-triazol-1,5-diyl | 1 | H | 2-chloro-phenyl |
| 28B | 5-carboxy-thiazol-4-yl | 1H-pyrazol-4,5-diyl | 4 | H | phenyl |
| 29B | 5-carboxy-thiazol-4-yl | 1H-pyrazol-4,5-diyl | 4 | H | 2-methyl-phenyl |
| 30B | 5-carboxy-thiazol-4-yl | 1H-pyrazol-4,5-diyl | 4 | H | 2-chloro-phenyl |
| 31B | 5-carboxy-oxazol-4-yl | isothiazol-3,4-diyl | 3 | H | phenyl |
| 32B | 5-carboxy-oxazol-4-yl | isothiazol-3,4-diyl | 3 | H | 2-methyl-phenyl |
| 33B | 5-carboxy-oxazol-4-yl | isothiazol-3,4-diyl | 3 | H | 2-chloro-phenyl |
| 34B | 5-carboxy-oxazol-4-yl | 1,2,3-triazol-4,5-diyl | 5 | H | phenyl |
| 35B | 5-carboxy-oxazol-4-yl | 1,2,3-triazol-4,5-diyl | 5 | H | 2-methyl-phenyl |
| 36B | 5-carboxy-oxazol-4-yl | 1,2,3-triazol-4,5-diyl | 5 | H | 2-chloro-phenyl |
| 37B | 5-carboxy-oxazol-4-yl | 1,2,3-triazol-1,5-diyl | 1 | H | phenyl |
| 38B | 5-carboxy-oxazol-4-yl | 1,2,3-triazol-1,5-diyl | 1 | H | 2-methyl-phenyl |
| 39B | 5-carboxy-oxazol-4-yl | 1,2,3-triazol-1,5-diyl | 1 | H | 2-chloro-phenyl |
| 40B | 5-carboxy-oxazol-4-yl | 1H-pyrazol-4,5-diyl | 4 | H | phenyl |
| 41B | 5-carboxy-oxazol-4-yl | 1H-pyrazol-4,5-diyl | 4 | H | 2-methyl-phenyl |
| 42B | 5-carboxy-oxazol-4-yl | 1H-pyrazol-4,5-diyl | 4 | H | 2-chloro-phenyl |
| 43B | 5-carboxy-oxazol-4-yl | 3-methyl isooxazol-4,5-diyl | 5 | H | 2-methyl-phenyl |
| 44B | 5-carboxy-oxazol-4-yl | 3-methyl isooxazol-4,5-diyl | 5 | H | 2-chloro-phenyl |
| 45B | 4-carboxy-thiazol-5-yl | isothiazol-3,4-diyl | 3 | methyl | phenyl |
| 46B | 4-carboxy-oxazol-5-yl | isothiazol-3,4-diyl | 3 | methyl | phenyl |
| 47B | 4-carboxy-thiazol-5-yl | 5-methyl isothiazol-3,4-diyl | 3 | H | phenyl |
| 48B | 4-carboxy-oxazol-5-yl | 5-methyl isothiazol-3,4-diyl | 3 | H | phenyl |
| 49B | 4-carboxy-thiazol-5-yl | 3-methyl isooxazol-4,5-diyl | 5 | methyl | phenyl |
| 50B | 4-carboxy-oxazol-5-yl | 3-methyl isooxazol-4,5-diyl | 5 | methyl | phenyl |
| 51B | 4-carboxy-thiazol-5-yl | isooxazol-3,4-diyl | 3 | H | phenyl |
| 52B | 4-carboxy-oxazol-5-yl | isooxazol-3,4-diyl | 3 | H | phenyl |
| 53B | 4-carboxy-thiazol-5-yl | isooxazol-3,4-diyl | 3 | H | 2-methyl-phenyl |
| 54B | 4-carboxy-oxazol-5-yl | isooxazol-3,4-diyl | 3 | H | 2-methyl-phenyl |
| 55B | 4-carboxy-thiazol-5-yl | isooxazol-3,4-diyl | 3 | H | 2-chloro-phenyl |
| 56B | 4-carboxy-oxazol-5-yl | isooxazol-3,4-diyl | 3 | H | 2-chloro-phenyl |
| 57B | 4-carboxy-thiazol-5-yl | isothiazol-3,4-diyl | 3 | H | 2-methyl-phenyl |
| 58B | 4-carboxy-thiazol-5-yl | isothiazol-3,4-diyl | 3 | H | 2-chloro-phenyl |
| 59B | 4-carboxy-thiazol-5-yl | isothiazol-4,5-diyl | 5 | H | phenyl |
| 60B | 4-carboxy-thiazol-5-yl | isothiazol-4,5-diyl | 5 | H | 2-methyl-phenyl |
| 61B | 4-carboxy-thiazol-5-yl | isothiazol-4,5-diyl | 5 | H | 2-chloro-phenyl |
| 62B | 4-carboxy-thiazol-5-yl | 1,2,3-triazol-4,5-diyl | 5 | H | phenyl |
| 63B | 4-carboxy-thiazol-5-yl | 1,2,3-triazol-4,5-diyl | 5 | H | 2-methyl-phenyl |
| 64B | 4-carboxy-thiazol-5-yl | 1,2,3-triazol-4,5-diyl | 5 | H | 2-chloro-phenyl |
| 65B | 4-carboxy-thiazol-5-yl | 1,2,3-triazol-1,5-diyl | 1 | H | phenyl |
| 66B | 4-carboxy-thiazol-5-yl | 1,2,3-triazol-1,5-diyl | 1 | H | 2-methyl-phenyl |
| 67B | 4-carboxy-thiazol-5-yl | 1,2,3-triazol-1,5-diyl | 1 | H | 2-chloro-phenyl |
| 68B | 4-carboxy-thiazol-5-yl | 1H-pyrazol-4,5-diyl | 4 | H | phenyl |
| 69B | 4-carboxy-thiazol-5-yl | 1H-pyrazol-4,5-diyl | 4 | H | 2-methyl-phenyl |
| 70B | 4-carboxy-thiazol-5-yl | 1H-pyrazol-4,5-diyl | 4 | H | 2-chloro-phenyl |
| 71B | 4-carboxy-thiazol-5-yl | 3-methyl isooxazol-4,5-diyl | 5 | H | 2-methyl-phenyl |
| 72B | 4-carboxy-thiazol-5-yl | 3-methyl isooxazol-4,5-diyl | 5 | H | 2-chloro-phenyl |
| 73B | 4-carboxy-oxazol-5-yl | isothiazol-3,4-diyl | 3 | H | 2-methyl-phenyl |
| 74B | 4-carboxy-oxazol-5-yl | isothiazol-3,4-diyl | 3 | H | 2-chloro-phenyl |
| 75B | 4-carboxy-oxazol-5-yl | isothiazol-4,5-diyl | 5 | H | phenyl |
| 76B | 4-carboxy-oxazol-5-yl | isothiazol-4,5-diyl | 5 | H | 2-methyl-phenyl |
| 77B | 4-carboxy-oxazol-5-yl | isothiazol-3,4-diyl | 3 | H | 2-chloro-phenyl |
| 78B | 4-carboxy-oxazol-5-yl | 1,2,3-triazol-4,5-diyl | 5 | H | phenyl |
| 79B | 4-carboxy-oxazol-5-yl | 1,2,3-triazol-4,5-diyl | 5 | H | 2-methyl-phenyl |
| 80B | 4-carboxy-oxazol-5-yl | 1,2,3-triazol-4,5-diyl | 5 | H | 2-chloro-phenyl |
| 81B | 4-carboxy-oxazol-5-yl | 1,2,3-triazol-1,5-diyl | 1 | H | phenyl |
| 82B | 4-carboxy-oxazol-5-yl | 1,2,3-triazol-1,5-diyl | 1 | H | 2-methyl-phenyl |
| 83B | 4-carboxy-oxazol-5-yl | 1,2,3-triazol-1,5-diyl | 1 | H | 2-chloro-phenyl |
| 84B | 4-carboxy-oxazol-5-yl | 1H-pyrazol-4,5-diyl | 4 | H | phenyl |
| 85B | 4-carboxy-oxazol-5-yl | 1H-pyrazol-4,5-diyl | 4 | H | 2-methyl-phenyl |
| 86B | 4-carboxy-oxazol-5-yl | 1H-pyrazol-4,5-diyl | 4 | H | 2-chloro-phenyl |
| 87B | 4-carboxy-oxazol-5-yl | 3-methyl isooxazol-4,5-diyl | 5 | H | 2-methyl-phenyl |
| 88B | 4-carboxy-oxazol-5-yl | 3-methyl isooxazol-4,5-diyl | 5 | H | 2-chloro-phenyl |

Note:
Column * indicates ring C's point of connection to the adjacent ring B.

In some embodiments, compounds of Formula (II) are also represented by Formula (II-C) and are selected from the following compounds as listed in Table 2C.

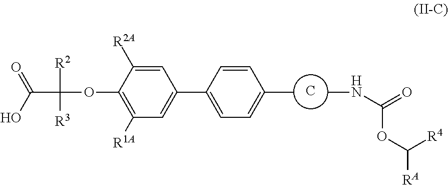

TABLE 2C

| Cmpd # II- | R²/R³ | 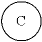 | * | R^{1A} | R^{2A} | R⁴ | R^{A} |
|---|---|---|---|---|---|---|---|
| 1C | cyclobutan-1,1-diyl | 1,2,3-triazol-4,5-diyl | 5 | H | H | CH₃ | phenyl |
| 2C | cyclopropan-1,1- | 3-methyl isooxazol-4,5-diyl | 5 | H | H | CH₃ | 2-methyl-phenyl |
| | diyl | | | | | | |
| 3C | cyclopentan-1,1-diyl | 3-methyl isooxazol-4,5-diyl | 5 | H | H | CH₃ | 2-methyl-phenyl |
| 4C | oxetan-3,3-diyl | 1,2,3-triazol-4,5-diyl | 5 | H | H | CH₃ | phenyl |
| 5C | cyclopropan-1,1- | 3-methyl isooxazol-4,5-diyl | 5 | H | H | H | phenyl |
| | diyl | | | | | | |
| 6C | cyclopentan-1,1-diyl | 3-methyl isooxazol-4,5-diyl | 5 | H | H | H | phenyl |
| 7C | cyclobutan-1,1-diyl | 1-methyl-1,2,3-triazol-4,5- | 4 | H | H | H | phenyl |
| | | diyl | | | | | |
| 8C | oxetan-3,3-diyl | 1-methyl-1,2,3-triazol-4,5- | 4 | H | H | H | phenyl |
| | | diyl | | | | | |
| 9C | cyclopropan-1,1- | 3-methyl isooxazol-4,5-diyl | 5 | H | H | CH₃ | phenyl |
| | diyl | | | | | | |
| 10C | cyclopentan-1,1-diyl | 3-methyl isooxazol-4,5-diyl | 5 | H | H | CH₃ | phenyl |
| 11C | cyclobutan-1,1-diyl | 3-methyl isooxazol-4,5-diyl | 5 | H | H | CH₃ | phenyl |
| 12C | oxetan-3,3-diyl | 3-methyl isooxazol-4,5-diyl | 5 | H | H | CH₃ | phenyl |
| 13C | cyclopropan-1,1- | isooxazol-4,5-diyl | 5 | H | H | H | 2-methyl-phenyl |
| | diyl | | | | | | |
| 14C | cyclopentan-1,1-diyl | isooxazol-4,5-diyl | 5 | H | H | H | 2-methyl-phenyl |
| 15C | cyclopentan-1,1-diyl | isooxazol-3,4-diyl | 3 | H | H | H | phenyl |
| 16C | cyclobutan-1,1-diyl | isooxazol-3,4-diyl | 3 | H | H | H | phenyl |
| 17C | cyclopropan-1,1- | isooxazol-3,4-diyl | 3 | H | H | H | phenyl |
| | diyl | | | | | | |
| 18C | oxetan-3,3-diyl | isooxazol-3,4-diyl | 3 | H | H | H | phenyl |
| 19C | cyclopropan-1,1- | isooxazol-3,4-diyl | 3 | H | H | H | 2-methyl-phenyl |
| | diyl | | | | | | |
| 20C | cyclobutan-1,1-diyl | isooxazol-3,4-diyl | 3 | H | H | H | 2-methyl-phenyl |
| 21C | cyclopentan-1,1-diyl | isooxazol-3,4-diyl | 3 | H | H | H | 2-methyl-phenyl |
| 22C | oxetan-3,3-diyl | isooxazol-3,4-diyl | 3 | H | H | H | 2-methyl-phenyl |
| 23C | cyclopentan-1,1-diyl | isooxazol-3,4-diyl | 3 | H | H | H | 2-chloro-phenyl |
| 24C | cyclobutan-1,1-diyl | isooxazol-3,4-diyl | 3 | H | H | H | 2-chloro-phenyl |
| 25C | cyclopropan-1,1- | isooxazol-3,4-diyl | 3 | H | H | H | 2-chloro-phenyl |
| | diyl | | | | | | |
| 26C | oxetan-3,3-diyl | isooxazol-3,4-diyl | 3 | H | H | H | 2-chloro-phenyl |
| 27C | cyclopropan-1,1- | 3-methyl isooxazol-4,5-diyl | 5 | H | H | H | 2-chloro-phenyl |
| | diyl | | | | | | |
| 28C | cyclopentan-1,1-diyl | 3-methyl isooxazol-4,5-diyl | 5 | H | H | H | 2-chloro-phenyl |
| 29C | cyclopropan-1,1- | isothiazol-3,4-diyl | 3 | H | H | H | phenyl |
| | diyl | | | | | | |
| 30C | cyclopentan-1,1-diyl | isothiazol-3,4-diyl | 3 | H | H | H | phenyl |
| 31C | oxetan-3,3-diyl | isothiazol-3,4-diyl | 3 | H | H | H | phenyl |
| 32C | cyclobutan-1,1-diyl | isothiazol-3,4-diyl | 3 | H | H | H | phenyl |
| 33C | cyclobutan-1,1-diyl | isothiazol-3,4-diyl | 3 | H | H | H | 2-methyl-phenyl |
| 34C | oxetan-3,3-diyl | isothiazol-3,4-diyl | 3 | H | H | H | 2-methyl-phenyl |
| 35C | cyclopropan-1,1- | isothiazol-3,4-diyl | 3 | H | H | H | 2-methyl-phenyl |
| | diyl | | | | | | |
| 36C | cyclopentan-1,1-diyl | isothiazol-3,4-diyl | 3 | H | H | H | 2-methyl-phenyl |
| 37C | cyclopropan-1,1- | isothiazol-3,4-diyl | 3 | H | H | H | 2-chloro-phenyl |
| | diyl | | | | | | |
| 38C | cyclopentan-1,1-diyl | isothiazol-3,4-diyl | 3 | H | H | H | 2-chloro-phenyl |
| 39C | cyclobutan-1,1-diyl | isothiazol-3,4-diyl | 3 | H | H | H | 2-chloro-phenyl |
| 40C | oxetan-3,3-diyl | isothiazol-3,4-diyl | 3 | H | H | H | 2-chloro-phenyl |
| 41C | cyclobutan-1,1-diyl | isothiazol-4,5-diyl | 5 | H | H | H | phenyl |
| 42C | cyclopropan-1,1- | isothiazol-4,5-diyl | 5 | H | H | H | phenyl |
| | diyl | | | | | | |
| 43C | cyclopentan-1,1-diyl | isothiazol-4,5-diyl | 5 | H | H | H | phenyl |
| 44C | oxetan-3,3-diyl | isothiazol-4,5-diyl | 5 | H | H | H | phenyl |
| 45C | cyclobutan-1,1-diyl | isothiazol-4,5-diyl | 5 | H | H | H | 2-methyl-phenyl |
| 46C | cyclopropan-1,1- | isothiazol-4,5-diyl | 5 | H | H | H | 2-methyl-phenyl |
| | diyl | | | | | | |
| 47C | cyclopentan-1,1-diyl | isothiazol-4,5-diyl | 5 | H | H | H | 2-methyl-phenyl |
| 48C | oxetan-3,3-diyl | isothiazol-4,5-diyl | 5 | H | H | H | 2-methyl-phenyl |
| 49C | cyclobutan-1,1-diyl | isothiazol-4,5-diyl | 5 | H | H | H | 2-chloro-phenyl |
| 50C | cyclopropan-1,1- | isothiazol-4,5-diyl | 5 | H | H | H | 2-chloro-phenyl |
| | diyl | | | | | | |
| 51C | cyclopentan-1,1-diyl | isothiazol-4,5-diyl | 5 | H | H | H | 2-chloro-phenyl |
| 52C | oxetan-3,3-diyl | isothiazol-4,5-diyl | 5 | H | H | H | 2-chloro-phenyl |
| 53C | cyclobutan-1,1-diyl | 1,2,3-triazol-4,5-diyl | 5 | H | H | H | 2-methyl-phenyl |
| 54C | cyclobutan-1,1-diyl | 1H-pyrazol-4,5-diyl | 4 | H | H | H | phenyl |
| 55C | cyclobutan-1,1-diyl | 1H-pyrazol-4,5-diyl | 4 | H | H | H | 2-chloro-phenyl |
| 56C | cyclobutan-1,1-diyl | 3-methyl isooxazol-4,5-diyl | 5 | H | H | H | 2-methyl-phenyl |
| 57C | cyclobutan-1,1-diyl | 3-methyl isooxazol-4,5-diyl | 5 | H | H | H | 2-chloro-phenyl |
| 58C | cyclobutan-1,1-diyl | 1,2,3-triazol-1,5-diyl | 1 | H | H | H | phenyl |
| 59C | cyclobutan-1,1-diyl | 1,2,3-triazol-1,5-diyl | 1 | H | H | H | 2-methyl-phenyl |
| 60C | cyclobutan-1,1-diyl | 1,2,3-triazol-1,5-diyl | 1 | H | H | H | 2-chloro-phenyl |
| 61C | cyclobutan-1,1-diyl | 1H-pyrazol-4,5-diyl | 4 | H | H | H | 2-methyl-phenyl |
| 62C | cyclopentan-1,1-diyl | 1,2,3-triazol-4,5-diyl | 5 | H | H | H | phenyl |
| 63C | cyclobutan-1,1-diyl | 1,2,3-triazol-4,5-diyl | 5 | H | H | H | 2-chloro-phenyl |
| 64C | cyclopropan-1,1- | 1,2,3-triazol-4,5-diyl | 5 | H | H | H | phenyl |
| | diyl | | | | | | |
| 65C | cyclopropan-1,1- | 1,2,3-triazol-4,5-diyl | 5 | H | H | H | 2-methyl-phenyl |
| | diyl | | | | | | |
| 66C | cyclopentan-1,1-diyl | 1,2,3-triazol-4,5-diyl | 5 | H | H | H | 2-methyl-phenyl |
| 67C | oxetan-3,3-diyl | 1,2,3-triazol-4,5-diyl | 5 | H | H | H | 2-methyl-phenyl |
| 68C | cyclopropan-1,1- | 1,2,3-triazol-4,5-diyl | 5 | H | H | H | 2-chloro-phenyl |
| | diyl | | | | | | |
| 69C | cyclopentan-1,1-diyl | 1,2,3-triazol-4,5-diyl | 5 | H | H | H | 2-chloro-phenyl |
| 70C | oxetan-3,3-diyl | 1,2,3-triazol-4,5-diyl | 5 | H | H | H | 2-chloro-phenyl |
| 71C | cyclopropan-1,1- | 1,2,3-triazol-1,5-diyl | 1 | H | H | H | phenyl |
| | diyl | | | | | | |
| 72C | cyclopentan-1,1-diyl | 1,2,3-triazol-1,5-diyl | 1 | H | H | H | phenyl |
| 73C | oxetan-3,3-diyl | 1,2,3-triazol-1,5-diyl | 1 | H | H | H | phenyl |
| 74C | oxetan-3,3-diyl | 1,2,3-triazol-1,5-diyl | 1 | H | H | H | 2-methyl-phenyl |
| 75C | cyclopropan-1,1- | 1,2,3-triazol-1,5-diyl | 1 | H | H | H | 2-methyl-phenyl |
| | diyl | | | | | | |
| 76C | cyclopentan-1,1-diyl | 1,2,3-triazol-1,5-diyl | 1 | H | H | H | 2-methyl-phenyl |
| 77C | cyclopropan-1,1- | 1,2,3-triazol-1,5-diyl | 1 | H | H | H | 2-chloro-phenyl |
| | diyl | | | | | | |
| 78C | cyclopentan-1,1-diyl | 1,2,3-triazol-1,5-diyl | 1 | H | H | H | 2-chloro-phenyl |
| 79C | oxetan-3,3-diyl | 1,2,3-triazol-1,5-diyl | 1 | H | H | H | 2-chloro-phenyl |
| 80C | cyclopropan-1,1- | 1H-pyrazol-4,5-diyl | 4 | H | H | H | phenyl |
| | diyl | | | | | | |
| 81C | cyclopentan-1,1-diyl | 1H-pyrazol-4,5-diyl | 4 | H | H | H | phenyl |
| 82C | oxetan-3,3-diyl | 1H-pyrazol-4,5-diyl | 4 | H | H | H | phenyl |
| 83C | cyclopropan-1,1- | 1H-pyrazol-4,5-diyl | 4 | H | H | H | 2-methyl-phenyl |
| | diyl | | | | | | |
| 84C | cyclopentan-1,1-diyl | 1H-pyrazol-4,5-diyl | 4 | H | H | H | 2-methyl-phenyl |
| 85C | oxetan-3,3-diyl | 1H-pyrazol-4,5-diyl | 4 | H | H | H | 2-methyl-phenyl |
| 86C | cyclopentan-1,1-diyl | 1H-pyrazol-4,5-diyl | 4 | H | H | H | 2-chloro-phenyl |
| 87C | oxetan-3,3-diyl | 1H-pyrazol-4,5-diyl | 4 | H | H | H | 2-chloro-phenyl |
| 88C | cyclopropan-1,1- | 1H-pyrazol-4,5-diyl | 4 | H | H | H | 2-chloro-phenyl |
| | diyl | | | | | | |
| 89C | oxetan-3,3-diyl | 3-methyl isooxazol-4,5-diyl | 5 | H | H | H | 2-methyl-phenyl |
| 90C | oxetan-3,3-diyl | 3-methyl isooxazol-4,5-diyl | 5 | H | H | H | 2-chloro-phenyl |
| 91C | CH₃/CH₃ | 3-methyl isooxazol-4,5-diyl | 5 | H | H | CH₃ | phenyl |
| 92C | CH₃/CH₃ | 3-methyl isooxazol-4,5-diyl | 5 | F | H | CH₃ | phenyl |
| 93C | CH₃/CH₃ | 3-methyl isooxazol-4,5-diyl | 5 | F | F | CH₃ | phenyl |
| 94C | CH₃/CH₃ | 3-methyl isothiazol-4,5-diyl | 5 | H | H | CH₃ | phenyl |
| 95C | CH₃/CH₃ | 3-methyl isothiazol-4,5-diyl | 5 | F | H | CH₃ | phenyl |
| 96C | CH₃/CH₃ | 3-methyl isothiazol-4,5-diyl | 5 | F | F | CH₃ | phenyl |
| 97C | CH₃/CH₃ | 1-methyl-pyrazol-4,5-diyl | 4 | H | H | CH₃ | phenyl |
| 98C | CH₃/CH₃ | 1-methyl-pyrazol-4,5-diyl | 4 | F | H | CH₃ | phenyl |
| 99C | CH₃/CH₃ | 1-methyl-pyrazol-4,5-diyl | 4 | F | F | CH₃ | Phenyl |

Note:
Column * indicates ring C's point of connection to the adjacent ring B.

In some embodiments, compounds of Formula (II) are also represented by Formula (II-D) and are selected from the following compounds as listed in Table 2D.

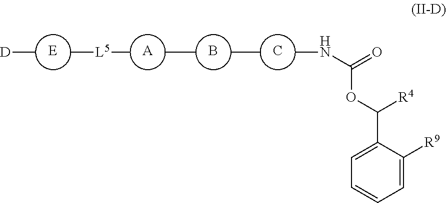

TABLE 2D

| Cmpd # II- | 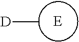 | L⁵ | 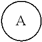 | 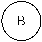 | 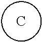 | * | R⁴ | R⁹ |
|---|---|---|---|---|---|---|---|---|
| 1D | 2-carboxy-phenyl | —O— | pyridin-2-one- | 1,4-phenylene | 1,2,3-triazol-4,5- | 5 | CH₃ | H |
| | | | 1,4-diyl | | diyl | | | |
| 2D | 2-carboxy-phenyl | —O— | pyridin-2-one- | 1,4-phenylene | 1,2,3-triazol-4,5- | 5 | CH₃ | CH₃ |
| | | | 1,4-diyl | | diyl | | | |
| 3D | 2-carboxy-phenyl | —O— | pyridin-2-one- | 1,4-phenylene | 1-methyl-1,2,3- | 4 | H | CH₃ |
| | | | 1,4-diyl | | triazol-4,5-diyl | | | |
| 4D | 2-carboxy-phenyl | —O— | pyridin-2-one- | 1,4-phenylene | 3-methyl | 5 | CH₃ | H |
| | | | 1,4-diyl | | isooxazol-4,5-diyl | | | |
| 5D | 2-carboxy-phenyl | —O— | pyridin-2-one- | 1,4-phenylene | 1,2,3-triazol-4,5- | 5 | H | H |
| | | | 1,4-diyl | | diyl | | | |
| 6D | 2-carboxy-phenyl | —O— | pyridin-2-one- | 1,4-phenylene | isooxazol-3,4-diyl | 3 | H | H |
| | | | 1,4-diyl | | | | | |
| 7D | 2-carboxy-phenyl | —O— | pyridin-2-one- | 1,4-phenylene | isooxazol-3,4-diyl | 3 | H | CH₃ |
| | | | 1,4-diyl | | | | | |
| 8D | 2-carboxy-phenyl | —O— | pyridin-2-one- | 1,4-phenylene | isooxazol-3,4-diyl | 3 | H | Cl |
| | | | 1,4-diyl | | | | | |
| 9D | 2-carboxy-phenyl | —O— | pyridin-2-one- | 1,4-phenylene | 1,2,3-triazol-4,5- | 5 | H | Cl |
| | | | 1,4-diyl | | diyl | | | |
| 10D | 2-carboxy-phenyl | —O— | pyridin-2-one- | 1,4-phenylene | isothiazol-3,4-diyl | 3 | H | H |
| | | | 1,4-diyl | | | | | |
| 11D | 4-carboxy-thiazol-5-yl | —O— | pyridin-2-one- | 1,4-phenylene | 1,2,3-triazol-4,5- | 5 | H | CH₃ |
| | | | 1,4-diyl | | diyl | | | |
| 12D | 2-carboxy-phenyl | —O— | pyridin-2-one- | 1,4-phenylene | isothiazol-3,4-diyl | 3 | H | CH₃ |
| | | | 1,4-diyl | | | | | |
| 13D | 2-carboxy-phenyl | —O— | pyridin-2-one- | 1,4-phenylene | isothiazol-3,4-diyl | 3 | H | Cl |
| | | | 1,4-diyl | | | | | |
| 14D | 2-carboxy-phenyl | —O— | pyridin-2-one- | 1,4-phenylene | isothiazol-4,5-diyl | 5 | H | H |
| | | | 1,4-diyl | | | | | |
| 15D | 2-carboxy-phenyl | —O— | pyridin-2-one- | 1,4-phenylene | isothiazol-4,5-diyl | 5 | H | CH₃ |
| | | | 1,4-diyl | | | | | |
| 16D | 2-carboxy-phenyl | —O— | pyridin-2-one- | 1,4-phenylene | isothiazol-4,5-diyl | 5 | H | Cl |
| | | | 1,4-diyl | | | | | |
| 17D | 2-carboxy-phenyl | —O— | pyridin-2-one- | 1,4-phenylene | 1,2,3-triazol-1,5- | 1 | H | Cl |
| | | | 1,4-diyl | | diyl | | | |
| 18D | 2-carboxy-phenyl | —O— | pyridin-2-one- | 1,4-phenylene | 1H-pyrazol-4,5- | 4 | H | H |
| | | | 1,4-diyl | | diyl | | | |
| 19D | 2-carboxy-phenyl | —O— | pyridin-2-one- | 1,4-phenylene | 1H-pyrazol-4,5- | 4 | H | CH₃ |
| | | | 1,4-diyl | | diyl | | | |
| 20D | 2-carboxy-phenyl | —O— | pyridin-2-one- | 1,4-phenylene | 1H-pyrazol-4,5- | 4 | H | Cl |
| | | | 1,4-diyl | | diyl | | | |
| 21D | 2-carboxy-phenyl | —O— | pyridin-2-one- | 1,4-phenylene | 3-methyl | 5 | H | CH₃ |
| | | | 1,4-diyl | | isooxazol-4,5-diyl | | | |
| 22D | 2-carboxy-phenyl | —O— | pyridin-2-one- | 1,4-phenylene | 3-methyl | 5 | H | Cl |
| | | | 1,4-diyl | | isooxazol-4,5-diyl | | | |
| 23D | 2-carboxy-phenyl | —O— | pyridin-2-one- | 1,4-phenylene | 1,2,3-triazol-1,5- | 1 | H | H |
| | | | 1,4-diyl | | diyl | | | |
| 24D | 2-carboxy-phenyl | —O— | pyridin-2-one- | 1,4-phenylene | 1,2,3-triazol-1,5- | 1 | H | CH₃ |
| | | | 1,4-diyl | | diyl | | | |
| 25D | 4-carboxy-pyridin-3-yl | —O— | pyridin-2-one- | 1,4-phenylene | 1,2,3-triazol-4,5- | 5 | H | CH₃ |
| | | | 1,4-diyl | | diyl | | | |
| 26D | 2-carboxy-pyridin-3-yl | —O— | pyridin-2-one- | 1,4-phenylene | 1,2,3-triazol-4,5- | 5 | H | CH₃ |
| | | | 1,4-diyl | | diyl | | | |
| 27D | 3-carboxy-pyridin-4-yl | —O— | pyridin-2-one- | 1,4-phenylene | 1,2,3-triazol-4,5- | 5 | H | CH₃ |
| | | | 1,4-diyl | | diyl | | | |
| 28D | 3-carboxy-pyridin-2-yl | —O— | pyridin-2-one- | 1,4-phenylene | 1,2,3-triazol-4,5- | 5 | H | CH₃ |
| | | | 1,4-diyl | | diyl | | | |
| 29D | 4-carboxy-oxazol-5-yl | —O— | pyridin-2-one- | 1,4-phenylene | 1,2,3-triazol-4,5- | 5 | H | CH₃ |
| | | | 1,4-diyl | | diyl | | | |
| 30D | 1-carboxy-cyclobutan- | —O— | pyridin-2-one- | 1,4-phenylene | 1,2,3-triazol-4,5- | 5 | H | CH₃ |
| | 1-yl | | 1,4-diyl | | diyl | | | |
| 31D | 1-carboxy- | —O— | pyridin-2-one- | 1,4-phenylene | 1,2,3-triazol-4,5- | 5 | H | CH₃ |
| | cyclopropan-1-yl | | 1,4-diyl | | diyl | | | |
| 32D | 1-carboxy- | —O— | pyridin-2-one- | 1,4-phenylene | 1,2,3-triazol-4,5- | 5 | H | CH₃ |
| | cyclopentan-1-yl | | 1,4-diyl | | diyl | | | |
| 33D | 3-carboxy-oxetan-3-yl | —O— | pyridin-2-one- | 1,4-phenylene | 1,2,3-triazol-4,5- | 5 | H | CH₃ |
| | | | 1,4-diyl | diyl | | | | |
| 34D | 2-carboxy-pyridin-3-yl | —O— | 1,4-phenylene | pyridin-2-one- | 1H-pyrazol-4,5- | 4 | H | CH₃ |
| | | | | 1,4-diyl | diyl | | | |
| 35D | 2-carboxy-pyridin-3-yl | —O— | pyridin-2-one- | 1,4-phenylene | isothiazol-3,4-diyl | 3 | H | H |
| | | | 1,4-diyl | | | | | |
| 36D | 4-carboxy-pyridin-3-yl | —O— | pyridin-2-one- | 1,4-phenylene | isothiazol-3,4-diyl | 3 | H | H |
| | | | 1,4-diyl | | | | | |
| 37D | 3-carboxy-pyridin-4-yl | —O— | pyridin-2-one- | 1,4-phenylene | isothiazol-3,4-diyl | 3 | H | H |
| | | | 1,4-diyl | | | | | |
| 38D | 3-carboxy-pyridin-2-yl | —O— | 1,4-phenylene | pyridin-2-one- | 1H-pyrazol-4,5- | 4 | H | CH₃ |
| | | | | 1,4-diyl | diyl | | | |
| 39D | 4-carboxy-pyridin-3-yl | —O— | 1,4-phenylene | pyridin-2-one- | 1H-pyrazol-4,5- | 4 | H | CH₃ |
| | | | | 1,4-diyl | diyl | | | |
| 40D | 3-carboxy-pyridin-4-yl | —O— | 1,4-phenylene | pyridin-2-one- | 1H-pyrazol-4,5- | 4 | H | CH₃ |
| | | | | 1,4-diyl | diyl | | | |
| 41D | 6-carboxy- | —O— | pyridin-2-one- | 1,4-phenylene | isothiazol-3,4-diyl | 3 | H | H |
| | benzo[d]thiazol-5-yl | | 1,4-diyl | | | | | |
| 42D | 4-carboxy- | —O— | pyridin-2-one- | 1,4-phenylene | isothiazol-3,4-diyl | 3 | H | H |
| | benzo[d]thiazol-5-yl | | 1,4-diyl | | | | | |
| 43D | 5-carboxy- | —O— | pyridin-2-one- | 1,4-phenylene | isothiazol-3,4-diyl | 3 | H | H |
| | benzo[d]oxazol-4-yl | | 1,4-diyl | | | | | |
| 44D | 5-carboxy- | —O— | pyridin-2-one- | 1,4-phenylene | isothiazol-3,4-diyl | 3 | H | H |
| | benzo[d]thiazol-4-yl | | 1,4-diyl | | | | | |
| 45D | 6-carboxy- | —O— | pyridin-2-one- | 1,4-phenylene | isothiazol-3,4-diyl | 3 | H | H |
| | benzo[d]oxazol-5-yl | | 1,4-diyl | | | | | |
| 46D | 4-carboxy- | —O— | pyridin-2-one- | 1,4-phenylene | isothiazol-3,4-diyl | 3 | H | H |
| | benzo[d]oxazol-5-yl | | 1,4-diyl | | | | | |
| 47D | 3-carboxy-pyridin-2-yl | —NH— | pyridin-2-one- | 1,4-phenylene | isothiazol-3,4-diyl | 3 | CH₃ | H |
| | | | 1,4-diyl | | | | | |
| 48D | 2-carboxy-phenyl | —S(O)₂— | 1,4-phenylene | pyridin-2-one- | 1H-pyrazol-4,5- | 4 | CH₃ | H |
| | | | | 1,4-diyl | diyl | | | |
| 49D | 2-carboxy-phenyl | —NH— | 1,4-phenylene | pyridin-2-one- | 1H-pyrazol-4,5- | 4 | CH₃ | H |
| | | | | 1,4-diyl | diyl | | | |
| 50D | 2-carboxy-phenyl | —S(O)₂— | 1,4-phenylene | pyridin-2-one- | 1H-pyrazol-4,5- | 4 | H | H |
| | | | | 1,4-diyl | diyl | | | |
| 51D | 2-carboxy-phenyl | —NH— | 1,4-phenylene | pyridin-2-one- | 1H-pyrazol-4,5- | 4 | H | H |
| | | | | 1,4-diyl | diyl | | | |
| 52D | 3-carboxy-pyridin-2-yl | —NH— | pyridin-2-one- | 1,4-phenylene | 5-methyl | 3 | H | H |
| | | | 1,4-diyl | | isothiazol-3,4-diyl | | | |
| 53D | 2-carboxy-phenyl | —S(O)₂— | 1,4-phenylene | pyridin-2-one- | 1-methyl-pyrazol- | 4 | H | CH₃ |
| | | | | 1,4-diyl | 4,5-diyl | | | |
| 54D | 2-carboxy-phenyl | —NH— | 1,4-phenylene | pyridin-2-one- | 1-methyl-pyrazol- | 4 | H | CH₃ |
| | | | | 1,4-diyl | 4,5-diyl | | | |
| 55D | 2-carboxy-phenyl | —S(O)₂— | 1,4-phenylene | pyridin-2-one- | 3-methyl | 5 | CH₃ | H |
| | | | | 1,4-diyl | isooxazol-4,5-diyl | | | |
| 56D | 2-carboxy-phenyl | —NH— | 1,4-phenylene | pyridin-2-one- | 3-methyl | 5 | CH₃ | H |
| | | | | 1,4-diyl | isooxazol-4,5-diyl | | | |
| 57D | 3-carboxy-pyridin-2-yl | —NH— | pyridin-2-one- | 1,4-phenylene | 3-methyl | 5 | CH₃ | H |
| | | | 1,4-diyl | | isooxazol-4,5-diyl | | | |
| 58D | 2-carboxy-pyridin-3-yl | —NH— | pyridin-2-one- | 1,4-phenylene | isothiazol-3,4-diyl | 3 | H | H |
| | | | 1,4-diyl | | | | | |
| 59D | 2-carboxy-phenyl | —S(O)₂— | 1,4-phenylene | pyridin-2-one- | isooxazol-3,4-diyl | 3 | H | H |
| | | | | 1,4-diyl | | | | |
| 60D | 2-carboxy-phenyl | —NH— | 1,4-phenylene | pyridin-2-one- | isooxazol-3,4-diyl | 3 | H | H |
| | | | | 1,4-diyl | | | | |
| 61D | 3-carboxy-pyridin-2-yl | —NH— | pyridin-2-one- | 1,4-phenylene | isooxazol-3,4-diyl | 3 | H | H |
| | | | 1,4-diyl | | | | | |
| 62D | 2-carboxy-phenyl | —NH— | pyridin-2-one- | 1,4-phenylene | isothiazol-3,4-diyl | 3 | H | H |
| | | | 1,4-diyl | | | | | |
| 63D | 3-carboxy-pyridin-2-yl | —NH— | pyridin-2-one- | 1,4-phenylene | isooxazol-3,4-diyl | 3 | H | CH₃ |
| | | | 1,4-diyl | | | | | |
| 64D | 2-carboxy-phenyl | —S(O)₂— | 1,4-phenylene | pyridin-2-one- | isooxazol-3,4-diyl | 3 | H | CH₃ |
| | | | | 1,4-diyl | | | | |
| 65D | 2-carboxy-phenyl | —NH— | 1,4-phenylene | pyridin-2-one- | isooxazol-3,4-diyl | 3 | H | CH₃ |
| | | | | 1,4-diyl | | | | |
| 66D | 2-carboxy-phenyl | —S(O)₂— | 1,4-phenylene | pyridin-2-one- | isooxazol-3,4-diyl | 3 | H | Cl |
| | | | | 1,4-diyl | | | | |
| 67D | 2-carboxy-phenyl | —NH— | 1,4-phenylene | pyridin-2-one- | isooxazol-3,4-diyl | 3 | H | Cl |
| | | | | 1,4-diyl | | | | |
| 68D | 3-carboxy-pyridin-2-yl | —NH— | pyridin-2-one- | 1,4-phenylene | isooxazol-3,4-diyl | 3 | H | Cl |
| | | | 1,4-diyl | | | | | |
| 69D | 3-carboxy-pyridin-2-yl | —NH— | pyridin-2-one- | 1,4-phenylene | isothiazol-3,4-diyl | 3 | H | CH₃ |
| | | | 1,4-diyl | | | | | |
| 70D | 2-carboxy-phenyl | —S(O)₂— | 1,4-phenylene | pyridin-2-one- | isothiazol-3,4-diyl | 3 | H | H |
| | | | | 1,4-diyl | | | | |
| 71D | 2-carboxy-phenyl | —NH— | 1,4-phenylene | pyridin-2-one- | isothiazol-3,4-diyl | 3 | H | H |
| | | | | 1,4-diyl | | | | |
| 72D | 3-carboxy-pyridin-2-yl | —NH— | pyridin-2-one- | 1,4-phenylene | 1H-pyrazol-4,5- | 4 | H | CH₃ |
| | | | 1,4-diyl | | diyl | | | |
| 73D | 3-carboxy-pyridin-2-yl | —NH— | pyridin-2-one- | 1,4-phenylene | 1H-pyrazol-4,5- | 4 | H | Cl |
| | | | 1,4-diyl | | diyl | | | |
| 74D | 3-carboxy-pyridin-2-yl | —NH— | pyridin-2-one- | 1,4-phenylene | 3-methyl | 5 | H | CH₃ |
| | | | 1,4-diyl | | isooxazol-4,5-diyl | | | |
| 75D | 3-carboxy-pyridin-2-yl | —NH— | pyridin-2-one- | 1,4-phenylene | isothiazol-3,4-diyl | 3 | H | Cl |
| | | | 1,4-diyl | | | | | |
| 76D | 3-carboxy-pyridin-2-yl | —NH— | pyridin-2-one- | 1,4-phenylene | isothiazol-4,5-diyl | 5 | H | H |
| | | | 1,4-diyl | | | | | |
| 77D | 3-carboxy-pyridin-2-yl | —NH— | pyridin-2-one- | 1,4-phenylene | isothiazol-4,5-diyl | 5 | H | CH₃ |
| | | | 1,4-diyl | | | | | |
| 78D | 3-carboxy-pyridin-2-yl | —NH— | pyridin-2-one- | 1,4-phenylene | isothiazol-4,5-diyl | 5 | H | Cl |
| | | | 1,4-diyl | | | | | |
| 79D | 3-carboxy-pyridin-2-yl | —NH— | pyridin-2-one- | 1,4-phenylene | 1,2,3-triazol-4,5- | 5 | H | H |
| | | | 1,4-diyl | | diyl | | | |
| 80D | 3-carboxy-pyridin-2-yl | —NH— | pyridin-2-one- | 1,4-phenylene | 1,2,3-triazol-4,5- | 5 | H | CH₃ |
| | | | 1,4-diyl | | diyl | | | |
| 81D | 3-carboxy-pyridin-2-yl | —NH— | pyridin-2-one- | 1,4-phenylene | 1,2,3-triazol-4,5- | 5 | H | Cl |
| | | | 1,4-diyl | | diyl | | | |
| 82D | 3-carboxy-pyridin-2-yl | —NH— | pyridin-2-one- | 1,4-phenylene | 1,2,3-triazol-1,5- | 1 | H | H |
| | | | 1,4-diyl | | diyl | | | |
| 83D | 3-carboxy-pyridin-2-yl | —NH— | pyridin-2-one- | 1,4-phenylene | 1,2,3-triazol-1,5- | 1 | H | CH₃ |
| | | | 1,4-diyl | | diyl | | | |
| 84D | 3-carboxy-pyridin-2-yl | —NH— | pyridin-2-one- | 1,4-phenylene | 1,2,3-triazol-1,5- | 1 | H | Cl |
| | | | 1,4-diyl | | diyl | | | |
| 85D | 3-carboxy-pyridin-2-yl | —NH— | pyridin-2-one- | 1,4-phenylene | 1H-pyrazol-4,5- | 4 | H | H |
| | | | 1,4-diyl | | diyl | | | |
| 86D | 3-carboxy-pyridin-2-yl | —NH— | pyridin-2-one- | 1,4-phenylene | 3-methyl | 5 | H | Cl |
| | | | 1,4-diyl | | isooxazol-4,5-diyl | | | |
| 87D | 2-carboxy-phenyl | —S— | pyridin-2-one- | 1,4-phenylene | isothiazol-3,4-diyl | 3 | H | H |
| | | | 1,4-diyl | | | | | |
| 88D | 3-carboxy-pyridin-2-yl | —NH— | 1,4-phenylene | pyridin-2-one- | 1H-pyrazol-4,5- | 4 | H | CH₃ |
| | | | | 1,4-diyl | diyl | | | |
| 89D | 4-carboxy-pyridin-3-yl | —NH— | pyridin-2-one- | 1,4-phenylene | isothiazol-3,4-diyl | 3 | H | H |
| | | | 1,4-diyl | | | | | |
| 90D | 3-carboxy-pyridin-4-yl | —NH— | pyridin-2-one- | 1,4-phenylene | isothiazol-3,4-diyl | 3 | H | H |
| | | | 1,4-diyl | | | | | |
| 91D | 2-carboxy-phenyl | —S(O)₂— | 1,4-phenylene | pyridin-2-one- | isothiazol-3,4-diyl | 3 | H | CH₃ |
| | | | | 1,4-diyl | | | | |
| 92D | 2-carboxy-phenyl | —NH— | 1,4-phenylene | pyridin-2-one- | isothiazol-3,4-diyl | 3 | H | CH₃ |
| | | | | 1,4-diyl | | | | |
| 93D | 2-carboxy-phenyl | —S(O)₂— | 1,4-phenylene | pyridin-2-one- | isothiazol-3,4-diyl | 3 | H | Cl |
| | | | | 1,4-diyl | | | | |
| 94D | 2-carboxy-phenyl | —NH— | 1,4-phenylene | pyridin-2-one- | isothiazol-3,4-diyl | 3 | H | Cl |
| | | | | 1,4-diyl | | | | |
| 95D | 2-carboxy-phenyl | —S(O)₂— | 1,4-phenylene | pyridin-2-one- | isothiazol-4,5-diyl | 5 | H | H |
| | | | | 1,4-diyl | | | | |
| 96D | 2-carboxy-phenyl | —NH— | 1,4-phenylene | pyridin-2-one- | isothiazol-4,5-diyl | 5 | H | H |
| | | | | 1,4-diyl | | | | |
| 97D | 2-carboxy-phenyl | —S(O)₂— | 1,4-phenylene | pyridin-2-one- | isothiazol-4,5-diyl | 5 | H | CH₃ |
| | | | | 1,4-diyl | | | | |
| 98D | 2-carboxy-phenyl | —NH— | 1,4-phenylene | pyridin-2-one- | isothiazol-4,5-diyl | 5 | H | CH₃ |
| | | | | 1,4-diyl | | | | |
| 99D | 2-carboxy-phenyl | —S(O)₂— | 1,4-phenylene | pyridin-2-one- | isothiazol-4,5-diyl | 5 | H | Cl |
| | | | | 1,4-diyl | | | | |
| 100D | 2-carboxy-phenyl | —NH— | 1,4-phenylene | pyridin-2-one- | isothiazol-4,5-diyl | 5 | H | Cl |
| | | | | 1,4-diyl | | | | |
| 101D | 2-carboxy-phenyl | —S(O)₂— | 1,4-phenylene | pyridin-2-one- | 1,2,3-triazol-4,5- | 5 | H | H |
| | | | | 1,4-diyl | diyl | | | |
| 102D | 2-carboxy-phenyl | —NH— | 1,4-phenylene | pyridin-2-one- | 1,2,3-triazol-4,5- | 5 | H | H |
| | | | | 1,4-diyl | diyl | | | |
| 103D | 2-carboxy-phenyl | —S(O)₂— | 1,4-phenylene | pyridin-2-one- | 1,2,3-triazol-4,5- | 5 | H | CH₃ |
| | | | | 1,4-diyl | diyl | | | |
| 104D | 2-carboxy-phenyl | —NH— | 1,4-phenylene | pyridin-2-one- | 1,2,3-triazol-4,5- | 5 | H | CH₃ |
| | | | | 1,4-diyl | diyl | | | |
| 105D | 2-carboxy-phenyl | —S(O)₂— | 1,4-phenylene | pyridin-2-one- | 1,2,3-triazol-4,5- | 5 | H | Cl |
| | | | | 1,4-diyl | diyl | | | |
| 106D | 2-carboxy-phenyl | —NH— | 1,4-phenylene | pyridin-2-one- | 1,2,3-triazol-4,5- | 5 | H | Cl |
| | | | | 1,4-diyl | diyl | | | |
| 107D | 2-carboxy-phenyl | —S(O)₂— | 1,4-phenylene | pyridin-2-one- | 1,2,3-triazol-1,5- | 1 | H | H |
| | | | | 1,4-diyl | diyl | | | |
| 108D | 2-carboxy-phenyl | —NH— | 1,4-phenylene | pyridin-2-one- | 1,2,3-triazol-1,5- | 1 | H | H |
| | | | | 1,4-diyl | diyl | | | |
| 109D | 2-carboxy-phenyl | —S(O)₂— | 1,4-phenylene | pyridin-2-one- | 1,2,3-triazol-1,5- | 1 | H | CH₃ |
| | | | | 1,4-diyl | diyl | | | |
| 110D | 2-carboxy-phenyl | —NH— | 1,4-phenylene | pyridin-2-one- | 1,2,3-triazol-1,5- | 1 | H | CH₃ |
| | | | | 1,4-diyl | diyl | | | |
| 111D | 2-carboxy-phenyl | —S(O)₂— | 1,4-phenylene | pyridin-2-one- | 1,2,3-triazol-1,5- | 1 | H | Cl |
| | | | | 1,4-diyl | diyl | | | |
| 112D | 2-carboxy-phenyl | —NH— | 1,4-phenylene | pyridin-2-one- | 1,2,3-triazol-1,5- | 1 | H | Cl |
| | | | | 1,4-diyl | diyl | | | |
| 113D | 2-carboxy-phenyl | —S— | 1,4-phenylene | pyridin-2-one- | 1H-pyrazol-4,5- | 4 | H | CH₃ |
| | | | | 1,4-diyl | diyl | | | |
| 114D | 2-carboxy-phenyl | —S(O)₂— | 1,4-phenylene | pyridin-2-one- | 1H-pyrazol-4,5- | 4 | H | Cl |
| | | | | 1,4-diyl | diyl | | | |
| 115D | 2-carboxy-phenyl | —NH— | 1,4-phenylene | pyridin-2-one- | 1H-pyrazol-4,5- | 4 | H | Cl |
| | | | | 1,4-diyl | diyl | | | |
| 116D | 2-carboxy-phenyl | —S(O)₂— | 1,4-phenylene | pyridin-2-one- | 3-methyl | 5 | H | CH₃ |
| | | | | 1,4-diyl | isooxazol-4,5-diyl | | | |
| 117D | 2-carboxy-phenyl | —NH— | 1,4-phenylene | pyridin-2-one- | 3-methyl | 5 | H | CH₃ |
| | | | | 1,4-diyl | isooxazol-4,5-diyl | | | |
| 118D | 2-carboxy-phenyl | —S(O)₂— | 1,4-phenylene | pyridin-2-one- | 3-methyl | 5 | H | Cl |
| | | | | 1,4-diyl | isooxazol-4,5-diyl | | | |
| 119D | 2-carboxy-phenyl | —NH— | 1,4-phenylene | pyridin-2-one- | 3-methyl | 5 | H | Cl |
| | | | | 1,4-diyl | isooxazol-4,5-diyl | | | |
| 120D | 6-carboxy- | —NH— | pyridin-2-one- | 1,4-phenylene | isothiazol-3,4-diyl | 3 | H | H |
| | benzo[d]thiazol-5-yl | | 1,4-diyl | | | | | |
| 121D | 6-carboxy- | —S(O)₂— | 1,4-phenylene | pyridin-2-one- | 1H-pyrazol-4,5- | 4 | H | CH₃ |
| | benzo[d]thiazol-5-yl | | 1,4-diyl | | diyl | | | |
| 122D | 6-carboxy- | —NH— | 1,4-phenylene | pyridin-2-one- | 1H-pyrazol-4,5- | 4 | H | CH₃ |
| | benzo[d]thiazol-5-yl | | | 1,4-diyl | diyl | | | |
| 123D | 2-carboxy-phenyl | —S(O)₂— | pyridin-2-one- | 1,4-phenylene | isothiazol-3,4-diyl | 3 | H | H |
| | | | 1,4-diyl | | | | | |
| 124D | 4-carboxy- | —NH— | pyridin-2-one- | 1,4-phenylene | isothiazol-3,4-diyl | 3 | H | H |
| | benzo[d]thiazol-5-yl | | 1,4-diyl | | | | | |
| 125D | 5-carboxy- | —NH— | pyridin-2-one- | 1,4-phenylene | isothiazol-3,4-diyl | 3 | H | H |
| | benzo[d]oxazol-4-yl | | 1,4-diyl | | | | | |
| 126D | 5-carboxy- | —NH— | pyridin-2-one- | 1,4-phenylene | isothiazol-3,4-diyl | 3 | H | H |
| | benzo[d]thiazol-4-yl | | 1,4-diyl | | | | | |
| 127D | 6-carboxy- | —NH— | pyridin-2-one- | 1,4-phenylene | isothiazol-3,4-diyl | 3 | H | H |
| | benzo[d]oxazol-5-yl | | 1,4-diyl | | | | | |
| 128D | 4-carboxy- | —NH— | pyridin-2-one- | 1,4-phenylene | isothiazol-3,4-diyl | 3 | H | H |
| | benzo[d]oxazol-5-yl | | 1,4-diyl | | | | | |
| 129D | 4-carboxy- | —S(O)₂— | 1,4-phenylene | pyridin-2-one- | 1H-pyrazol-4,5- | 4 | H | CH₃ |
| | benzo[d]thiazol-5-yl | | 1,4-diyl | diyl | | | | |
| 130D | 4-carboxy- | —NH— | 1,4-phenylene | pyridin-2-one- | 1H-pyrazol-4,5- | 4 | H | CH₃ |
| | benzo[d]thiazol-5-yl | | | 1,4-diyl | diyl | | | |
| 131D | 6-carboxy- | —S(O)₂— | 1,4-phenylene | pyridin-2-one- | 1H-pyrazol-4,5- | 4 | H | CH₃ |
| | benzo[d]oxazol-5-yl | | | 1,4-diyl | diyl | | | |
| 132D | 6-carboxy- | —NH— | 1,4-phenylene | pyridin-2-one- | 1H-pyrazol-4,5- | 4 | H | CH₃ |
| | benzo[d]oxazol-5-yl | | | 1,4-diyl | diyl | | | |
| 133D | 2-carboxy-phenyl | —S(O)₂— | 1,4-phenylene | pyridin-2-one- | 1H-pyrazol-4,5- | 4 | H | CH₃ |
| | | | | 1,4-diyl | diyl | | | |
| 134D | 5-carboxy- | —S(O)₂— | 1,4-phenylene | pyridin-2-one- | 1H-pyrazol-4,5- | 4 | H | CH₃ |
| | benzo[d]oxazol-4-yl | | | 1,4-diyl | diyl | | | |
| 135D | 5-carboxy- | —NH— | 1,4-phenylene | pyridin-2-one- | 1H-pyrazol-4,5- | 4 | H | CH₃ |
| | benzo[d]oxazol-4-yl | | | 1,4-diyl | diyl | | | |
| 136D | 5-carboxy- | —S(O)₂— | 1,4-phenylene | pyridin-2-one- | 1H-pyrazol-4,5- | 4 | H | CH₃ |
| | benzo[d]thiazol-4-yl | | | 1,4-diyl | diyl | | | |
| 137D | 5-carboxy- | —NH— | 1,4-phenylene | pyridin-2-one- | 1H-pyrazol-4,5- | 4 | H | CH₃ |
| | benzo[d]thiazol-4-yl | | | 1,4-diyl | diyl | | | |
| 138D | 4-carboxy- | —S(O)₂— | 1,4-phenylene | pyridin-2-one- | 1H-pyrazol-4,5- | 4 | H | CH₃ |
| | benzo[d]oxazol-5-yl | | | 1,4-diyl | diyl | | | |
| 139D | 4-carboxy- | —NH— | 1,4-phenylene | pyridin-2-one- | 1H-pyrazol-4,5- | 4 | H | CH₃ |
| | benzo[d]oxazol-5-yl | | | 1,4-diyl | diyl | | | |

Note:
Column * indicates ring C's point of connection to the adjacent ring B.

In some embodiments, compounds of Formula (III) are selected from the following compounds as listed in Table 3.

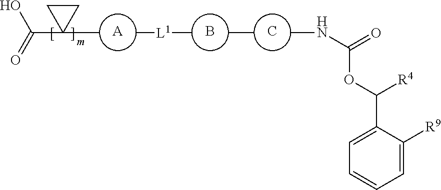

TABLE 3

| Cmpd # III- | m | 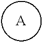 | ++ | L¹ | 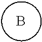 | # | 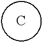 | * | R⁴ | R⁹ |
|---|---|---|---|---|---|---|---|---|---|---|
| 1 | 1 | 1,4-phenylene | 4 | — | cyclohexane- | 4 | 3-methyl | 5 | CH₃ | H |
| | | | | | 1,4- | | isooxazol-4,5- | | | |
| | | | | | diyl | | diyl | | | |
| 2 | 1 | cyclohexane-1,4-diyl | 4 | — | 1,4- | 4 | 3-methyl | 5 | CH₃ | H |
| | | | | | phenylene | | isooxazol-4,5- | | | |
| | | | | | | | diyl | | | |
| 3 | 1 | 1,4-phenylene | 4 | — | cyclohexane- | 4 | 3-methyl | 5 | CH₃ | Cl |
| | | | | | 1,4- | | isooxazol-4,5- | | | |
| | | | | | diyl | | diyl | | | |
| 4 | 1 | cyclohexane-1,4-diyl | 4 | — | 1,4- | 4 | 3-methyl | 5 | CH₃ | Cl |
| | | | | | phenylene | | isooxazol-4,5- | | | |
| | | | | | | | diyl | | | |
| 5 | 1 | 1,4-phenylene | 4 | — | cyclohexane- | 4 | 3-methyl | 5 | CH₃ | Cl |
| | | | | | 1,4- | | isooxazol-4,5- | | | |
| | | | | | diyl | | diyl | | | |
| 6 | 1 | 1,4-phenylene | 4 | — | cyclohexane- | 4 | 3-methyl | 5 | CH₃ | CH₃ |
| | | | | | 1,4- | | isooxazol-4,5- | | | |
| | | | | | diyl | | diyl | | | |
| 7 | 0 | 1H-indol-2,7-diol | 7 | — | 1,4- | 4 | 3-methyl | 5 | CH₃ | H |
| | | | | | phenylene | | isooxazol-4,5- | | | |
| | | | | | | | diyl | | | |
| 8 | 1 | 1H-indol-2,7-diol | 7 | — | 1,4- | 4 | 3-methyl | 5 | CH₃ | H |
| | | | | | phenylene | | isooxazol-4,5- | | | |
| | | | | | | | diyl | | | |
| 9 | 1 | 1H-indol-2,6-diol | 6 | — | 1,4- | 4 | isooxazol-4,5- | 5 | CH₃ | H |
| | | | | | phenylene | | diyl | | | |
| 10 | 0 | 1H-indol-2,6-diol | 6 | — | 1,4- | 4 | 3-methyl | 5 | CH₃ | H |
| | | | | | phenylene | | isooxazol-4,5- | | | |
| | | | | | | | diyl | | | |
| 11 | 0 | 1H-indol-2,6-diol | 6 | — | 1,4- | 4 | 3-methyl | 5 | CH₃ | H |
| | | | | | phenylene | | isooxazol-4,5- | | | |
| | | | | | | | diyl | | | |
| 12 | 1 | thien-2,5-diyl | 4 | — | 1,4- | 4 | 3-methyl- | 5 | CH₃ | H |
| | | | | | phenylene | | pyrazol-4,5-diyl | | | |
| 13 | 1 | thien-2,5-diyl | 4 | — | 1,4- | 4 | 3-methyl | 5 | CH₃ | H |
| | | | | | phenylene | | isooxazol-4,5- | | | |
| | | | | | | | diyl | | | |
| 14 | 1 | thien-2,5-diyl | 4 | — | 1,4- | 4 | 3-methyl | 5 | CH₃ | H |
| | | | | | phenylene | | isooxazol-4,5- | | | |
| | | | | | | | diyl | | | |
| 15 | 1 | imidazol-2,4-diyl | 4 | — | 1,4- | 4 | 3-methyl- | 5 | CH₃ | H |
| | | | | | phenylene | | pyrazol-4,5-diyl | | | |
| 16 | 1 | imidazol-2,4-diyl | 4 | — | 1,4- | 4 | 3-methyl | 5 | CH₃ | H |
| | | | | | phenylene | | isooxazol-4,5- | | | |
| | | | | | | | diyl | | | |
| 17 | 1 | imidazol-2,4-diyl | 4 | — | 1,4- | 4 | 3-methyl | 5 | CH₃ | H |
| | | | | | phenylene | | isooxazol-4,5- | | | |
| | | | | | | | diyl | | | |
| 18 | 1 | oxazol-2,5-diyl | 5 | — | 1,4- | 4 | 3-methyl- | 5 | CH₃ | H |
| | | | | | phenylene | | pyrazol-4,5-diyl | | | |
| 19 | 1 | oxazol-2,5-diyl | 5 | — | 1,4- | 4 | 3-methyl | 5 | CH₃ | H |
| | | | | | phenylene | | isooxazol-4,5- | | | |
| | | | | | | | diyl | | | |
| 20 | 1 | oxazol-2,5-diyl | 5 | — | 1,4- | 4 | 3-methyl | 5 | CH₃ | H |
| | | | | | phenylene | | isooxazol-4,5- | | | |
| | | | | | | | diyl | | | |
| 21 | 1 | thiazol-2,5-diyl | 5 | — | 1,4- | 4 | 3-methyl- | 5 | CH₃ | H |
| | | | | | phenylene | | pyrazol-4,5-diyl | | | |
| 22 | 1 | thiazol-2,5-diyl | 5 | — | 1,4- | 4 | 3-methyl | 5 | CH₃ | H |
| | | | | | phenylene | | isooxazol-4,5- | | | |
| | | | | | | | diyl | | | |
| 23 | 1 | thiazol-2,5-diyl | 5 | — | 1,4- | 4 | 3-methyl | 5 | CH₃ | H |
| | | | | | phenylene | | isooxazol-4,5- | | | |
| | | | | | | | diyl | | | |
| 24 | 1 | imidazolidin-2-on-1,3- | 3 | — | 1,4- | 4 | 3-methyl- | 5 | CH₃ | H |
| | | diyl | | | phenylene | | pyrazol-4,5-diyl | | | |
| 25 | 1 | imidazolidin-2-on-1,3- | 3 | — | 1,4- | 4 | 3-methyl | 5 | CH₃ | H |
| | | diyl | | | phenylene | | isooxazol-4,5- | | | |
| | | | | | | | diyl | | | |
| 26 | 1 | imidazolidin-2-on-1,3- | 3 | — | 1,4- | 4 | 3-methyl | 5 | CH₃ | H |
| | | diyl | | | phenylene | | isooxazol-4,5- | | | |
| | | | | | | | diyl | | | |
| 27 | 0 | thien-2,5-diyl | 5 | — | naphthalen- | 6 | 3-methyl- | 5 | CH₃ | H |
| | | | | | 2,6-diyl | | pyrazol-4,5-diyl | | | |
| 28 | 0 | thien-2,5-diyl | 5 | — | naphthalen- | 6 | 3-methyl | 5 | CH₃ | H |
| | | | | | 2,6-diyl | | isooxazol-4,5- | | | |
| | | | | | | | diyl | | | |
| 29 | 0 | thien-2,5-diyl | 5 | — | naphthalen- | 6 | 3-methyl | 5 | CH₃ | H |
| | | | | | 2,6-diyl | | isooxazol-4,5- | | | |
| | | | | | | | diyl | | | |
| 30 | 0 | thiazol-2,5-diyl | 2 | — | naphthalen- | 6 | 1-methyl- | 4 | CH₃ | H |
| | | | | | 2,6-diyl | | pyrazol-4,5-diyl | | | |
| 31 | 0 | oxazol-2,5-diyl | 2 | — | naphthalen- | 6 | 3-methyl | 5 | CH₃ | H |
| | | | | | 2,6-diyl | | isooxazol-4,5- | | | |
| | | | | | | | diyl | | | |
| 32 | 0 | imidazol-2,4-diyl | 2 | — | naphthalen- | 6 | 3-methyl | 5 | CH₃ | H |
| | | | | | 2,6-diyl | | isooxazol-4,5- | | | |
| | | | | | | | diyl | | | |
| 33 | 0 | thiazol-2,5-diyl | 5 | — | naphthalen- | 6 | 1-methyl- | 5 | CH₃ | H |
| | | | | | 2,6-diyl | | pyrazol-4,5-diyl | | | |
| 34 | 0 | oxazol-2,5-diyl | 5 | — | naphthalen- | 6 | 3-methyl | 5 | CH₃ | H |
| | | | | | 2,6-diyl | | isooxazol-4,5- | | | |
| | | | | | | | diyl | | | |
| 35 | 0 | imidazol-2,4-diyl | 4 | — | naphthalen- | 6 | 3-methyl | 5 | CH₃ | H |
| | | | | | 2,6-diyl | | isooxazol-4,5- | | | |
| | | | | | | | diyl | | | |
| 36 | 0 | pyrazol-3,5-diyl | 3 | — | naphthalen- | 6 | 3-methyl | 5 | CH₃ | H |
| | | | | | 2,6-diyl | | isooxazol-4,5- | | | |
| | | | | | | | diyl | | | |
| 37 | 0 | 1,2,4-triazol-3,5-diyl | 3 | — | naphthalen- | 6 | 3-methyl | 5 | CH₃ | H |
| | | | | | 2,6-diyl | | isooxazol-4,5- | | | |
| | | | | | | | diyl | | | |
| 38 | 1 | piperazine-2,5-dion-1,4- | 4 | — | 1,4- | 4 | 3-methyl | 5 | CH₃ | H |
| | | diyl | | | phenylene | | isooxazol-4,5- | | | |
| | | | | | | | diyl | | | |
| 39 | 1 | piperazine-2,5-dion-1,4- | 4 | — | 1,4- | 4 | 3-methyl | 5 | CH₃ | Cl |
| | | diyl | | | phenylene | | isooxazol-4,5- | | | |
| | | | | | | | diyl | | | |
| 40 | 1 | 1,4-phenylene | 4 | — | piperazine- | 4 | 3-methyl | 5 | CH₃ | H |
| | | | | | 2,5-dion- | | isooxazol-4,5- | | | |
| | | | | | 1,4-diyl | | diyl | | | |
| 41 | 1 | 1,4-phenylene | 4 | — | piperazine- | 4 | 3-methyl | 5 | CH₃ | Cl |
| | | | | | 2,5-dion- | | isooxazol-4,5- | | | |
| | | | | | 1,4-diyl | | diyl | | | |
| 42 | 1 | piperazine-2,5-dion-1,4- | 4 | — | 1,4- | 4 | 3-methyl | 5 | CH₃ | H |
| | | diyl | | | phenylene | | isooxazol-4,5- | | | |
| | | | | | | | diyl | | | |
| 43 | 1 | piperazine-2,5-dion-1,4- | 4 | — | 1,4- | 4 | 3-methyl | 5 | CH₃ | Cl |
| | | diyl | | | phenylene | | isooxazol-4,5- | | | |
| | | | | | | | diyl | | | |
| 44 | 1 | 1,4-phenylene | 4 | — | piperazine- | 4 | 3-methyl | 5 | CH₃ | H |
| | | | | | 2,5-dion- | | isooxazol-4,5- | | | |
| | | | | | 1,4-diyl | | diyl | | | |
| 45 | 1 | 1,4-phenylene | 4 | — | piperazine- | 4 | 3-methyl | 5 | CH₃ | Cl |
| | | | | | 2,5-dion- | | isooxazol-4,5- | | | |
| | | | | | 1,4-diyl | | diyl | | | |
| 46 | 1 | piperazine-2,5-dion-1,4- | 4 | — | 1,4- | 4 | 1-methyl- | 5 | CH₃ | H |
| | | diyl | | | phenylene | | pyrazol-4,5-diyl | | | |
| 47 | 1 | piperazine-2,5-dion-1,4- | 4 | — | 1,4- | 4 | 1-methyl- | 5 | CH₃ | Cl |
| | | diyl | | | phenylene | | pyrazol-4,5-diyl | | | |
| 48 | 1 | 1,4-phenylene | 4 | — | piperazine- | 4 | 1-methyl- | 5 | CH₃ | H |
| | | | | | 2,5-dion- | | pyrazol-4,5-diyl | | | |
| | | | | | 1,4-diyl | | 1-methyl- | | | |
| 49 | 1 | 1,4-phenylene | 4 | — | piperazine- | 4 | pyrazol-4,5-diyl | 5 | CH₃ | Cl |
| | | | | | 2,5-dion- | | 1-methyl- | | | |
| | | | | | 1,4-diyl | | | | | |
| 50 | 0 | 1H-indol-3,6-diyl | 3 | — | 1,4- | 4 | 3-methyl | 5 | CH₃ | H |
| | | | | | phenylene | | isooxazol-4,5- | | | |
| | | | | | | | diyl | | | |
| 51 | 0 | benzofuran-3,6-diyl | 3 | — | 1,4- | 4 | 3-methyl | 5 | CH₃ | H |
| | | | | | phenylene | | isooxazol-4,5- | | | |
| | | | | | | | diyl | | | |
| 52 | 0 | thieno[3,2-b]thiophene- | 5 | — | 1,4- | 4 | 3-methyl | 5 | CH₃ | H |
| | | 2,5-diyl | | | phenylene | | isooxazol-4,5- | | | |
| | | | | | | | diyl | | | |
| 53 | 1 | thieno[3,2-b]thiophene- | 5 | — | 1,4- | 4 | 3-methyl | 5 | CH₃ | H |
| | | 2,5-diyl | | | phenylene | | isooxazol-4,5- | | | |
| | | | | | | | diyl | | | |
| 54 | 1 | 1,4-phenylene | 4 | NHC(═O) | imidazol- | 1 | 3-methyl | 5 | CH₃ | H |
| | | | | | 1,4-diyl | | isooxazol-4,5- | | | |
| | | | | | | | diyl | | | |
| 55 | 1 | 1,4-phenylene | 4 | NHC(═O) | imidazol- | 1 | 3-methyl | 5 | CH₃ | Cl |
| | | | | | 1,4-diyl | | isooxazol-4,5- | | | |
| | | | | | | | diyl | | | |
| 56 | 1 | 1,4-phenylene | 4 | NHC(═O) | imidazol- | 1 | 3-methyl | 5 | CH₃ | H |
| | | | | | 1,4-diyl | | isooxazol-4,5- | | | |
| | | | | | | | diyl | | | |
| 57 | 1 | 1,4-phenylene | 4 | NHC(═O) | imidazol- | 1 | 3-methyl | 5 | CH₃ | CH₃ |
| | | | | | 1,4-diyl | | isooxazol-4,5- | | | |
| | | | | | | | diyl | | | |
| 58 | 1 | 1,4-phenylene | 4 | NHC(═O) | imidazol- | 2 | 3-methyl | 5 | CH₃ | H |
| | | | | | 1,4-diyl | | isooxazol-4,5- | | | |
| | | | | | | | diyl | | | |
| 59 | 1 | 1,4-phenylene | 4 | NHC(═O) | imidazol- | 2 | 3-methyl | 5 | CH₃ | H |
| | | | | | 1,4-diyl | | isooxazol-4,5- | | | |
| | | | | | | | diyl | | | |
| 60 | 1 | 1,4-phenylene | 4 | NHC(═O) | imidazol- | 2 | 3-methyl | 5 | CH₃ | Cl |
| | | | | | 1,4-diyl | | isooxazol-4,5- | | | |
| | | | | | | | diyl | | | |
| 61 | 1 | 2,3-dihydrothieno[2,3- | 2 | — | 1,4- | 4 | 3-methyl | 5 | CH₃ | H |
| | | d]isothiazol-2,5-diyl 1,1- | | | phenylene | | isooxazol-4,5- | | | |
| | | dioxide | | | | | diyl | | | |
| 62 | 1 | 2,3-dihydrothieno[2,3- | 2 | — | 1,4- | 4 | 3-methyl | 5 | CH₃ | CH₃ |
| | | d]isothiazol-2,5-diyl 1,1- | | | phenylene | | isooxazol-4,5- | | | |
| | | dioxide | | | | | diyl | | | |
| 63 | 1 | 2,3-dihydrothieno[2,3- | 2 | — | 1,4- | 4 | 3-methyl | 5 | CH₃ | Cl |
| | | d]isothiazol-2,5-diyl 1,1- | | | phenylene | | isooxazol-4,5- | | | |
| | | dioxide | | | | | diyl | | | |
| 64 | 1 | 2,3-dihydrothieno[2,3- | 2 | — | 1,4- | 4 | isooxazol-4,5- | 5 | CH₃ | H |
| | | d]isothiazol-2,5-diyl 1,1- | | | phenylene | | diyl | | | |
| | | dioxide | | | | | | | | |
| 65 | 1 | 2,3-dihydrothieno[2,3- | 2 | — | 1,4- | 4 | isooxazol-4,5- | 5 | CH₃ | CH₃ |
| | | d]isothiazol-2,5-diyl 1,1- | | | phenylene | | diyl | | | |
| | | dioxide | | | | | | | | |
| 66 | 1 | 2,3-dihydrothieno[2,3- | 2 | — | 1,4- | 4 | isooxazol-4,5- | 5 | CH₃ | Cl |
| | | d]isothiazol-2,5-diyl 1,1- | | | phenylene | | diyl | | | |
| | | dioxide | | | | | | | | |
| 67 | 1 | 2,3-dihydrothieno[2,3- | 2 | — | 1,4- | 4 | 3-methyl | 5 | H | H |
| | | d]isothiazol-2,5-diyl 1,1- | | | phenylene | | isooxazol-4,5- | | | |
| | | dioxide | | | | | diyl | | | |
| 68 | 1 | 2,3-dihydrothieno[2,3- | 2 | — | 1,4- | 4 | 3-methyl | 5 | H | CH₃ |
| | | d]isothiazol-2,5-diyl 1,1- | | | phenylene | | isooxazol-4,5- | | | |
| | | dioxide | | | | | diyl | | | |
| 69 | 1 | 2,3-dihydrothieno[2,3- | 2 | — | 1,4- | 4 | 3-methyl | 5 | H | Cl |
| | | d]isothiazol-2,5-diyl 1,1- | | | phenylene | | isooxazol-4,5- | | | |
| | | dioxide | | | | | diyl | | | |
| 70 | 1 | 2,3-dihydrothieno[2,3- | 2 | — | 1,4- | 4 | isooxazol-4,5- | 5 | H | H |
| | | d]isothiazol-2,5-diyl 1,1- | | | phenylene | | diyl | | | |
| | | dioxide | | | | | | | | |
| 71 | 1 | 2,3-dihydrothieno[2,3- | 2 | — | 1,4- | 4 | isooxazol-4,5- | 5 | H | CH₃ |
| | | d]isothiazol-2,5-diyl 1,1- | | | phenylene | | diyl | | | |
| | | dioxide | | | | | | | | |
| 72 | 1 | 2,3-dihydrothieno[2,3- | 2 | — | 1,4- | 4 | isooxazol-4,5- | 5 | H | Cl |
| | | d]isothiazol-2,5-diyl 1,1- | | | phenylene | | diyl | | | |
| | | dioxide | | | | | | | | |
| 73 | 1 | 2,3-dihydrothieno[2,3- | 2 | — | 1,4- | 4 | 3-methyl | 5 | CH₃ | H |
| | | d]isothiazol-2,5-diyl 1,1- | | | phenylene | | isooxazol-4,5- | | | |
| | | dioxide | | | | | diyl | | | |
| 74 | 1 | 2,3-dihydrothieno[2,3- | 2 | — | 1,4- | 4 | 3-methyl | 5 | CH₃ | CH₃ |
| | | d]isothiazol-2,5-diyl 1,1- | | | phenylene | | isooxazol-4,5- | | | |
| | | dioxide | | | | | diyl | | | |
| 75 | 1 | 2,3-dihydrothieno[2,3- | 2 | — | 1,4- | 4 | 3-methyl | 5 | CH₃ | Cl |
| | | d]isothiazol-2,5-diyl 1,1- | | | phenylene | | isooxazol-4,5- | | | |
| | | dioxide | | | | | diyl | | | |
| 76 | 1 | 2,3-dihydrothieno[2,3- | 2 | — | 1,4- | 4 | isothiazol-4,5- | 5 | CH₃ | H |
| | | d]isothiazol-2,5-diyl 1,1- | | | phenylene | | diyl | | | |
| | | dioxide | | | | | | | | |
| 77 | 1 | 2,3-dihydrothieno[2,3- | 2 | — | 1,4- | 4 | isothiazol-4,5- | 5 | CH₃ | CH₃ |
| | | d]isothiazol-2,5-diyl 1,1- | | | phenylene | | diyl | | | |
| | | dioxide | | | | | | | | |
| 78 | 1 | 2,3-dihydrothieno[2,3- | 2 | — | 1,4- | 4 | isothiazol-4,5- | 5 | CH₃ | Cl |
| | | d]isothiazol-2,5-diyl 1,1- | | | phenylene | | diyl | | | |
| | | dioxide | | | | | | | | |
| 79 | 1 | 2,3-dihydrothieno[2,3- | 2 | — | 1,4- | 4 | 3-methyl | 5 | H | H |
| | | d]isothiazol-2,5-diyl 1,1- | | | phenylene | | isooxazol-4,5- | | | |
| | | dioxide | | | | | diyl | | | |
| 80 | 1 | 2,3-dihydrothieno[2,3- | 2 | — | 1,4- | 4 | 3-methyl | 5 | H | CH₃ |
| | | d]isothiazol-2,5-diyl 1,1- | | | phenylene | | isooxazol-4,5- | | | |
| | | dioxide | | | | | diyl | | | |
| 81 | 1 | 2,3-dihydrothieno[2,3- | 2 | — | 1,4- | 4 | 3-methyl | 5 | H | Cl |
| | | d]isothiazol-2,5-diyl 1,1- | | | phenylene | | isooxazol-4,5- | | | |
| | | dioxide | | | | | diyl | | | |
| 82 | 1 | 2,3-dihydrothieno[2,3- | 2 | — | 1,4- | 4 | isothiazol-4,5- | 5 | H | H |
| | | d]isothiazol-2,5-diyl 1,1- | | | phenylene | | diyl | | | |
| | | dioxide | | | | | | | | |
| 83 | 1 | 2,3-dihydrothieno[2,3- | 2 | — | 1,4- | 4 | isothiazol-4,5- | 5 | H | CH₃ |
| | | d]isothiazol-2,5-diyl 1,1- | | | phenylene | | diyl | | | |
| | | dioxide | | | | | | | | |
| 84 | 1 | 2,3-dihydrothieno[2,3- | 2 | — | 1,4- | 4 | isothiazol-4,5- | 5 | H | Cl |
| | | d]isothiazol-2,5-diyl 1,1- | | | phenylene | | diyl | | | |
| | | dioxide | | | | | | | | |
| 85 | 1 | 2,3-dihydrothieno[2,3- | 2 | — | 1,4- | 4 | 5-methyl | 3 | CH₃ | H |
| | | d]isothiazol-2,5-diyl 1,1- | | | phenylene | | isooxazol-3,4- | | | |
| | | dioxide | | | | | diyl | | | |
| 86 | 1 | 2,3-dihydrothieno[2,3- | 2 | — | 1,4- | 4 | 5-methyl | 3 | CH₃ | CH₃ |
| | | d]isothiazol-2,5-diyl 1,1- | | | phenylene | | isooxazol-3,4- | | | |
| | | dioxide | | | | | diyl | | | |
| 87 | 1 | 2,3-dihydrothieno[2,3- | 2 | — | 1,4- | 4 | 5-methyl | 3 | CH₃ | Cl |
| | | d]isothiazol-2,5-diyl 1,1- | | | phenylene | | isooxazol-3,4- | | | |
| | | dioxide | | | | | diyl | | | |
| 88 | 1 | 2,3-dihydrothieno[2,3- | 2 | — | 1,4- | 4 | 5-methyl | 3 | H | H |
| | | d]isothiazol-2,5-diyl 1,1- | | | phenylene | | isooxazol-3,4- | | | |
| | | dioxide | | | | | diyl | | | |
| 89 | 1 | 2,3-dihydrothieno[2,3- | 2 | — | 1,4- | 4 | 5-methyl | 3 | H | CH₃ |
| | | d]isothiazol-2,5-diyl 1,1- | | | phenylene | | isooxazol-3,4- | | | |
| | | dioxide | | | | | diyl | | | |
| 90 | 1 | 2,3-dihydrothieno[2,3- | 2 | — | 1,4- | 4 | 5-methyl | 3 | H | Cl |
| | | d]isothiazol-2,5-diyl 1,1- | | | phenylene | | isooxazol-3,4- | | | |
| | | dioxide | | | | | diyl | | | |
| 91 | 1 | 2,3-dihydrothieno[2,3- | 2 | — | 1,4- | 4 | 1-methyl- | 4 | CH₃ | H |
| | | d]isothiazol-2,5-diyl 1,1- | | | phenylene | | pyrazol-4,5-diyl | | | |
| | | dioxide | | | | | | | | |
| 92 | 1 | 2,3-dihydrothieno[2,3- | 2 | — | 1,4- | 4 | 1-methyl- | 4 | CH₃ | CH₃ |
| | | d]isothiazol-2,5-diyl 1,1- | | | phenylene | | pyrazol-4,5-diyl | | | |
| | | dioxide | | | | | | | | |
| 93 | 1 | 2,3-dihydrothieno[2,3- | 2 | — | 1,4- | 4 | 1-methyl- | 4 | CH₃ | Cl |
| | | d]isothiazol-2,5-diyl 1,1- | | | phenylene | | pyrazol-4,5-diyl | | | |
| | | dioxide | | | | | | | | |
| 94 | 1 | 2,3-dihydrothieno[2,3- | 2 | — | 1,4- | 4 | 1-methyl- | 4 | H | H |
| | | d]isothiazol-2,5-diyl 1,1- | | | phenylene | | pyrazol-4,5-diyl | | | |
| | | dioxide | | | | | | | | |
| 95 | 1 | 2,3-dihydrothieno[2,3- | 2 | — | 1,4- | 4 | 1-methyl- | 4 | H | CH₃ |
| | | d]isothiazol-2,5-diyl 1,1- | | | phenylene | | pyrazol-4,5-diyl | | | |
| | | dioxide | | | | | | | | |
| 96 | 1 | 2,3-dihydrothieno[2,3- | 2 | — | 1,4- | 4 | 1-methyl- | 4 | H | Cl |
| | | d]isothiazol-2,5-diyl 1,1- | | | phenylene | | pyrazol-4,5-diyl | | | |
| | | dioxide | | | | | | | | |
| 97 | 1 | 2,3-dihydrothieno[2,3- | 2 | — | 1,4- | 4 | isooxazol-3,4- | 3 | CH₃ | H |
| | | d]isothiazol-2,5-diyl 1,1- | | | phenylene | | diyl | | | |
| | | dioxide | | | | | | | | |
| 98 | 1 | 2,3-dihydrothieno[2,3- | 2 | — | 1,4- | 4 | isooxazol-3,4- | 3 | CH₃ | CH₃ |
| | | d]isothiazol-2,5-diyl 1,1- | | | phenylene | | diyl | | | |
| | | dioxide | | | | | | | | |
| 99 | 1 | 2,3-dihydrothieno[2,3- | 2 | — | 1,4- | 4 | isooxazol-3,4- | 3 | CH₃ | Cl |
| | | d]isothiazol-2,5-diyl 1,1- | | | phenylene | | diyl | | | |
| | | dioxide | | | | | | | | |
| 100 | 1 | 2,3-dihydrothieno[2,3- | 2 | — | 1,4- | 4 | isooxazol-3,4- | 3 | H | H |
| | | d]isothiazol-2,5-diyl 1,1- | | | phenylene | | diyl | | | |
| | | dioxide | | | | | | | | |
| 101 | 1 | 2,3-dihydrothieno[2,3- | 2 | — | 1,4- | 4 | isooxazol-3,4- | 3 | H | CH₃ |
| | | d]isothiazol-2,5-diyl 1,1- | | | phenylene | | diyl | | | |
| | | dioxide | | | | | | | | |
| 102 | 1 | 2,3-dihydrothieno[2,3- | 2 | — | 1,4- | 4 | isooxazol-3,4- | 3 | H | Cl |
| | | d]isothiazol-2,5-diyl 1,1- | | | phenylene | | diyl | | | |
| | | dioxide | | | | | | | | |
| 103 | 1 | 2,3-dihydrothieno[2,3- | 2 | — | 1,4- | 4 | 1H-pyrazol-4,5- | 4 | CH₃ | H |
| | | d]isothiazol-2,5-diyl 1,1- | | | phenylene | | diyl | | | |
| | | dioxide | | | | | | | | |
| 104 | 1 | 2,3-dihydrothieno[2,3- | 2 | — | 1,4- | 4 | 1H-pyrazol-4,5- | 4 | CH₃ | CH₃ |
| | | d]isothiazol-2,5-diyl 1,1- | | | phenylene | | diyl | | | |
| | | dioxide | | | | | | | | |
| 105 | 1 | 2,3-dihydrothieno[2,3- | 2 | — | 1,4- | 4 | 1H-pyrazol-4,5- | 4 | CH₃ | Cl |
| | | d]isothiazol-2,5-diyl 1,1- | | | phenylene | | diyl | | | |
| | | dioxide | | | | | | | | |
| 106 | 1 | 2,3-dihydrothieno[2,3- | 2 | — | 1,4- | 4 | 1H-pyrazol-4,5- | 4 | H | H |
| | | d]isothiazol-2,5-diyl 1,1- | | | phenylene | | diyl | | | |
| | | dioxide | | | | | | | | |
| 107 | 1 | 2,3-dihydrothieno[2,3- | 2 | — | 1,4- | 4 | 1H-pyrazol-4,5- | 4 | H | CH₃ |
| | | d]isothiazol-2,5-diyl 1,1- | | | phenylene | | diyl | | | |
| | | dioxide | | | | | | | | |
| 108 | 1 | 2,3-dihydrothieno[2,3- | 2 | — | 1,4- | 4 | 1H-pyrazol-4,5- | 4 | H | Cl |
| | | d]isothiazol-2,5-diyl 1,1- | | | phenylene | | diyl | | | |
| | | dioxide | | | | | | | | |
| 109 | 1 | 2,3-dihydrothieno[2,3- | 2 | — | 1,4- | 4 | 1,2,5-oxadiazol- | 3 | CH₃ | H |
| | | d]isothiazol-2,5-diyl 1,1- | | | phenylene | | 3,4-diyl | | | |
| | | dioxide | | | | | | | | |
| 110 | 1 | 2,3-dihydrothieno[2,3- | 2 | — | 1,4- | 4 | 1,2,5-oxadiazol- | 3 | CH₃ | CH₃ |
| | | d]isothiazol-2,5-diyl 1,1- | | | phenylene | | 3,4-diyl | | | |
| | | dioxide | | | | | | | | |
| 111 | 1 | 2,3-dihydrothieno[2,3- | 2 | — | 1,4- | 4 | 1,2,5-oxadiazol- | 3 | CH₃ | Cl |
| | | d]isothiazol-2,5-diyl 1,1- | | | phenylene | | 3,4-diyl | | | |
| | | dioxide | | | | | | | | |
| 112 | 1 | 2,3-dihydrothieno[2,3- | 2 | — | 1,4- | 4 | 1,2,5-oxadiazol- | 3 | H | H |
| | | d]isothiazol-2,5-diyl 1,1- | | | phenylene | | 3,4-diyl | | | |
| | | dioxide | | | | | | | | |
| 113 | 1 | 2,3-dihydrothieno[2,3- | 2 | — | 1,4- | 4 | 1,2,5-oxadiazol- | 3 | H | CH₃ |
| | | d]isothiazol-2,5-diyl 1,1- | | | phenylene | | 3,4-diyl | | | |
| | | dioxide | | | | | | | | |
| 114 | 1 | 2,3-dihydrothieno[2,3- | 2 | — | 1,4- | 4 | 1,2,5-oxadiazol- | 3 | H | Cl |
| | | d]isothiazol-2,5-diyl 1,1- | | | phenylene | | 3,4-diyl | | | |
| | | dioxide | | | | | | | | |
| 115 | 1 | 2,3-dihydrothieno[2,3- | 2 | — | 1,4- | 4 | 1-methyl-1,2,3- | 4 | CH₃ | H |
| | | d]isothiazol-2,5-diyl 1,1- | | | phenylene | | triazol-4,5-diyl | | | |
| | | dioxide | | | | | | | | |
| 116 | 1 | 2,3-dihydrothieno[2,3- | 2 | — | 1,4- | 4 | 1-methyl-1,2,3- | 4 | CH₃ | CH₃ |
| | | d]isothiazol-2,5-diyl 1,1- | | | phenylene | | triazol-4,5-diyl | | | |
| | | dioxide | | | | | | | | |
| 117 | 1 | 2,3-dihydrothieno[2,3- | 2 | — | 1,4- | 4 | 1-methyl-1,2,3- | 4 | CH₃ | Cl |
| | | d]isothiazol-2,5-diyl 1,1- | | | phenylene | | triazol-4,5-diyl | | | |
| | | dioxide | | | | | | | | |
| 118 | 1 | 2,3-dihydrothieno[2,3- | 2 | — | 1,4- | 4 | 1-methyl-1,2,3- | 4 | H | H |
| | | d]isothiazol-2,5-diyl 1,1- | | | phenylene | | triazol-4,5-diyl | | | |
| | | dioxide | | | | | | | | |
| 119 | 1 | 2,3-dihydrothieno[2,3- | 2 | — | 1,4- | 4 | 1-methyl-1,2,3- | 4 | H | CH₃ |
| | | d]isothiazol-2,5-diyl 1,1- | | | phenylene | | triazol-4,5-diyl | | | |
| | | dioxide | | | | | | | | |
| 120 | 1 | 2,3-dihydrothieno[2,3- | 2 | — | 1,4- | 4 | 1-methyl-1,2,3- | 4 | H | Cl |
| | | d]isothiazol-2,5-diyl 1,1- | | | phenylene | | triazol-4,5-diyl | | | |
| | | dioxide | | | | | | | | |

Note:
Column ++ indicates ring A's point of connection to the adjacent terminal moiety.
Column # indicates ring B's point of connection to the adjacent L¹ moiety.
Column * indicates ring C's point of connection to the adjacent ring B.
When L¹ is —NHC(=O), the carbonyl is connected to the adjacent ring B.

In some embodiments, compounds of Formula (III) are also represented by Formula (III-A) and are selected from the following compounds as listed in Table 3A.

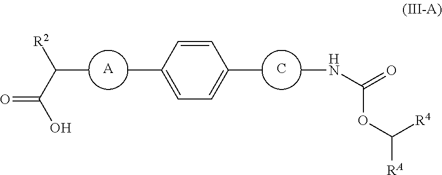

TABLE 3A

| Cmpd # III- | R² | 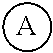 | 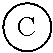 | * | R⁴ | R^{A} |
|---|---|---|---|---|---|---|
| 1A | 263oxetane-3-yl | pyridin-2-one-1,4-diyl | 1,2,3-triazol-4,5-diyl | 5 | H | 2-methyl-phenyl |
| 2A | cyclopropanyl | pyridin-2-one-1,4-diyl | 1,2,3-triazol-4,5-diyl | 5 | H | 2-methyl-phenyl |
| 3A | thiazol-4-yl | pyridin-2-one-1,4-diyl | 1,2,3-triazol-4,5-diyl | 5 | H | 2-methyl-phenyl |
| 4A | cyclobutanyl | pyridin-2-one-1,4-diyl | 1,2,3-triazol-4,5-diyl | 5 | H | 2-methyl-phenyl |
| 5A | oxazol-4-yl | pyridin-2-one-1,4-diyl | 1,2,3-triazol-4,5-diyl | 5 | H | 2-methyl-phenyl |
| 6A | cyclohexanyl | pyridin-2-one-1,4-diyl | 1,2,3-triazol-4,5-diyl | 5 | H | 2-methyl-phenyl |
| 7A | cyclopentanyl | pyridin-2-one-1,4-diyl | 1,2,3-triazol-4,5-diyl | 5 | H | 2-methyl-phenyl |
| 8A | 263oxetane-3-yl | pyridin-2-one-1,4-diyl | 1,2,3-triazol-4,5-diyl | 5 | CH₃ | 2-methyl-phenyl |
| 9A | cyclopropanyl | pyridin-2-one-1,4-diyl | 1,2,3-triazol-4,5-diyl | 5 | CH₃ | 2-methyl-phenyl |
| 10A | thiazol-4-yl | pyridin-2-one-1,4-diyl | 1,2,3-triazol-4,5-diyl | 5 | CH₃ | 2-methyl-phenyl |
| 11A | cyclobutanyl | pyridin-2-one-1,4-diyl | 1,2,3-triazol-4,5-diyl | 5 | CH₃ | 2-methyl-phenyl |
| 12A | oxazol-4-yl | pyridin-2-one-1,4-diyl | 1,2,3-triazol-4,5-diyl | 5 | CH₃ | 2-methyl-phenyl |
| 13A | cyclohexanyl | pyridin-2-one-1,4-diyl | 1,2,3-triazol-4,5-diyl | 5 | CH₃ | 2-methyl-phenyl |
| 14A | cyclopentanyl | pyridin-2-one-1,4-diyl | 1,2,3-triazol-4,5-diyl | 5 | CH₃ | 2-methyl-phenyl |

Note:
Column * indicates ring C's point of connection to the adjacent ring phen-1,4-ylene.

In some embodiments, compounds of Formula (III) are also represented by Formula (III-B) and are selected from the following compounds as listed in Table 3B.

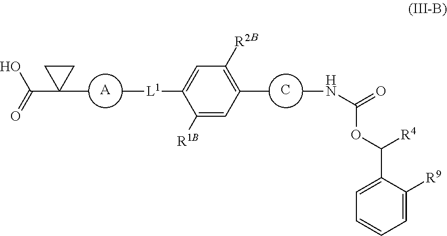

TABLE 3B

| Cmpd # III- | 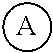 | L¹ | # | 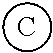 | * | R^{1B} | R^{2B} | R⁴ | R⁹ |
|---|---|---|---|---|---|---|---|---|---|
| 1B | thiazol-2,4-diyl | C(═O)NCH₃ | 4 | 1H-pyrazol-4,5-diyl | 4 | H | H | CH₃ | H |
| 2B | thiazol-2,4-diyl | C(═O)NH | 2 | 1H-pyrazol-4,5-diyl | 4 | H | H | CH₃ | H |
| 3B | oxazol-2,4-diyl | C(═O)NH | 2 | 1H-pyrazol-4,5-diyl | 4 | H | H | CH₃ | H |
| 4B | oxazol-2,4-diyl | C(═O)NH | 4 | 1H-pyrazol-4,5-diyl | 4 | H | H | CH₃ | H |
| 5B | furan-2,4-diyl | C(═O)NH | 2 | 1H-pyrazol-4,5-diyl | 4 | H | H | CH₃ | H |
| 6B | thien-2,4-diyl | C(═O)NH | 2 | 1H-pyrazol-4,5-diyl | 4 | H | H | CH₃ | H |
| 7B | thien-2,4-diyl | C(═O)NCH₃ | 2 | 1H-pyrazol-4,5-diyl | 4 | H | H | CH₃ | H |
| 8B | thien-2,4-diyl | NHC(═O) | 2 | 1H-pyrazol-4,5-diyl | 4 | H | H | CH₃ | H |
| 9B | thiazol-2,4-diyl | C(═O)NH | 4 | 1H-pyrazol-4,5-diyl | 4 | H | H | CH₃ | H |
| 10B | thiazol-2,4-diyl | C(═O)NCH₃ | 4 | 1-methyl-pyrazol-4,5-diyl | 4 | H | H | H | CH₃ |
| 11B | thiazol-2,4-diyl | C(═O)NH | 2 | 1-methyl-pyrazol-4,5-diyl | 4 | H | H | H | CH₃ |
| 12B | oxazol-2,4-diyl | C(═O)NH | 2 | 1-methyl-pyrazol-4,5-diyl | 4 | H | H | H | CH₃ |
| 13B | oxazol-2,4-diyl | C(═O)NH | 4 | 1-methyl-pyrazol-4,5-diyl | 4 | H | H | H | CH₃ |
| 14B | furan-2,4-diyl | C(═O)NH | 2 | 1-methyl-pyrazol-4,5-diyl | 4 | H | H | H | CH₃ |
| 15B | thien-2,4-diyl | C(═O)NH | 2 | 1-methyl-pyrazol-4,5-diyl | 4 | H | H | H | CH₃ |
| 16B | thien-2,4-diyl | NCH₃C(═O) | 2 | 1-methyl-pyrazol-4,5-diyl | 4 | H | H | H | CH₃ |
| 17B | thien-2,4-diyl | NHC(═O) | 2 | 1-methyl-pyrazol-4,5-diyl | 4 | H | H | H | CH₃ |
| 18B | thiazol-2,4-diyl | C(═O)NH | 4 | 1-methyl-pyrazol-4,5-diyl | 4 | H | H | H | CH₃ |
| 19B | thiazol-2,4-diyl | C(═O)NCH₃ | 2 | 3-methyl isooxazol-4,5-diyl | 5 | H | H | CH₃ | H |
| 20B | thiazol-2,4-diyl | C(═O)NCH₃ | 4 | 3-methyl isooxazol-4,5-diyl | 5 | H | H | CH₃ | H |
| 21B | thiazol-2,4-diyl | C(═O)NH | 4 | 3-methyl isooxazol-4,5-diyl | 5 | H | H | CH₃ | H |
| 22B | thiazol-2,4-diyl | C(═O)NH | 2 | 3-methyl isooxazol-4,5-diyl | 5 | H | H | CH₃ | H |
| 23B | oxazol-2,4-diyl | C(═O)NH | 4 | 3-methyl isooxazol-4,5-diyl | 5 | H | H | CH₃ | H |
| 24B | oxazol-2,4-diyl | C(═O)NH | 2 | 3-methyl isooxazol-4,5-diyl | 5 | H | H | CH₃ | H |
| 25B | furan-2,4-diyl | C(═O)NH | 2 | 3-methyl isooxazol-4,5-diyl | 5 | H | H | CH₃ | H |
| 26B | thien-2,4-diyl | C(═O)NH | 2 | 3-methyl isooxazol-4,5-diyl | 5 | H | H | CH₃ | H |
| 27B | thien-2,4-diyl | NCH₃C(═O) | 2 | 3-methyl isooxazol-4,5-diyl | 5 | H | H | CH₃ | H |
| 28B | thien-2,4-diyl | NHC(═O) | 2 | 3-methyl isooxazol-4,5-diyl | 5 | H | H | CH₃ | H |
| 29B | thiazol-2,4-diyl | C(═O)NCH₃ | 2 | isooxazol-3,4-diyl | 3 | H | H | H | H |
| 30B | thiazol-2,4-diyl | C(═O)NCH₃ | 4 | isooxazol-3,4-diyl | 3 | H | H | H | H |
| 31B | thiazol-2,4-diyl | C(═O)NH | 4 | isooxazol-3,4-diyl | 3 | H | H | H | H |
| 32B | thiazol-2,4-diyl | C(═O)NH | 2 | isooxazol-3,4-diyl | 3 | H | H | H | H |
| 33B | thien-2,4-diyl | C(═O)NH | 2 | isooxazol-3,4-diyl | 3 | H | H | H | H |
| 34B | thien-2,4-diyl | NCH₃C(═O) | 2 | isooxazol-3,4-diyl | 3 | H | H | H | H |
| 35B | thien-2,4-diyl | NHC(═O) | 2 | isooxazol-3,4-diyl | 3 | H | H | H | H |
| 36B | oxazol-2,4-diyl | C(═O)NH | 4 | isooxazol-3,4-diyl | 3 | H | H | H | H |
| 37B | oxazol-2,4-diyl | C(═O)NH | 2 | isooxazol-3,4-diyl | 3 | H | H | H | H |
| 38B | furan-2,4-diyl | C(═O)NH | 2 | isooxazol-3,4-diyl | 3 | H | H | H | H |
| 39B | thiazol-2,4-diyl | C(═O)NCH₃ | 2 | isooxazol-3,4-diyl | 3 | H | H | H | CH₃ |
| 40B | thiazol-2,4-diyl | C(═O)NCH₃ | 4 | isooxazol-3,4-diyl | 3 | H | H | H | CH₃ |
| 41B | thiazol-2,4-diyl | C(═O)NH | 4 | isooxazol-3,4-diyl | 3 | H | H | H | CH₃ |
| 42B | thiazol-2,4-diyl | C(═O)NH | 2 | isooxazol-3,4-diyl | 3 | H | H | H | CH₃ |
| 43B | oxazol-2,4-diyl | C(═O)NH | 2 | isooxazol-3,4-diyl | 3 | H | H | H | CH₃ |
| 44B | oxazol-2,4-diyl | C(═O)NH | 4 | isooxazol-3,4-diyl | 3 | H | H | H | CH₃ |
| 45B | furan-2,4-diyl | C(═O)NH | 2 | isooxazol-3,4-diyl | 3 | H | H | H | CH₃ |
| 46B | thien-2,4-diyl | C(═O)NH | 2 | isooxazol-3,4-diyl | 3 | H | H | H | CH₃ |
| 47B | thien-2,4-diyl | NCH₃C(═O) | 2 | isooxazol-3,4-diyl | 3 | H | H | H | CH₃ |
| 48B | thien-2,4-diyl | NHC(═O) | 2 | isooxazol-3,4-diyl | 3 | H | H | H | CH₃ |
| 49B | thiazol-2,4-diyl | C(═O)NCH₃ | 2 | isooxazol-3,4-diyl | 3 | H | H | H | Cl |
| 50B | thiazol-2,4-diyl | C(═O)NCH₃ | 4 | isooxazol-3,4-diyl | 3 | H | H | H | Cl |
| 51B | thiazol-2,4-diyl | C(═O)NH | 4 | isooxazol-3,4-diyl | 3 | H | H | H | Cl |
| 52B | thiazol-2,4-diyl | C(═O)NH | 2 | isooxazol-3,4-diyl | 3 | H | H | H | Cl |
| 53B | oxazol-2,4-diyl | C(═O)NH | 4 | isooxazol-3,4-diyl | 3 | H | H | H | Cl |
| 54B | oxazol-2,4-diyl | C(═O)NH | 2 | isooxazol-3,4-diyl | 3 | H | H | H | Cl |
| 55B | thien-2,4-diyl | C(═O)NH | 2 | isooxazol-3,4-diyl | 3 | H | H | H | Cl |
| 56B | thien-2,4-diyl | NCH₃C(═O) | 2 | isooxazol-3,4-diyl | 3 | H | H | H | Cl |
| 57B | thien-2,4-diyl | C(═O)NH | 2 | 1H-pyrazol-4,5-diyl | 4 | H | H | H | Cl |
| 58B | thiazol-2,4-diyl | C(═O)NH | 4 | 1H-pyrazol-4,5-diyl | 4 | H | H | H | Cl |
| 59B | furan-2,4-diyl | C(═O)NH | 2 | isooxazol-3,4-diyl | 3 | H | H | H | Cl |
| 60B | thien-2,4-diyl | NHC(═O) | 2 | isooxazol-3,4-diyl | 3 | H | H | H | Cl |
| 61B | thiazol-2,4-diyl | C(═O)NH | 2 | 1H-pyrazol-4,5-diyl | 4 | H | H | H | Cl |
| 62B | oxazol-2,4-diyl | C(═O)NH | 2 | 1H-pyrazol-4,5-diyl | 4 | H | H | H | Cl |
| 63B | thiazol-2,4-diyl | C(═O)NCH₃ | 2 | isothiazol-3,4-diyl | 3 | H | H | H | H |
| 64B | thiazol-2,4-diyl | C(═O)NCH₃ | 4 | isothiazol-3,4-diyl | 3 | H | H | H | H |
| 65B | thiazol-2,4-diyl | C(═O)NH | 4 | isothiazol-3,4-diyl | 3 | H | H | H | H |
| 66B | thiazol-2,4-diyl | C(═O)NH | 2 | isothiazol-3,4-diyl | 3 | H | H | H | H |
| 67B | thien-2,4-diyl | C(═O)NH | 2 | isothiazol-3,4-diyl | 3 | H | H | H | H |
| 68B | thien-2,4-diyl | NCH₃C(═O) | 2 | isothiazol-3,4-diyl | 3 | H | H | H | H |
| 69B | oxazol-2,4-diyl | C(═O)NH | 4 | isothiazol-3,4-diyl | 3 | H | H | H | H |
| 70B | oxazol-2,4-diyl | C(═O)NH | 2 | isothiazol-3,4-diyl | 3 | H | H | H | H |
| 71B | furan-2,4-diyl | C(═O)NH | 2 | isothiazol-3,4-diyl | 3 | H | H | H | H |
| 72B | thien-2,4-diyl | NHC(═O) | 2 | isothiazol-3,4-diyl | 3 | H | H | H | H |
| 73B | thiazol-2,4-diyl | C(═O)NCH₃ | 2 | isothiazol-3,4-diyl | 3 | H | H | H | CH₃ |
| 74B | thiazol-2,4-diyl | C(═O)NCH₃ | 4 | isothiazol-3,4-diyl | 3 | H | H | H | CH₃ |
| 75B | thiazol-2,4-diyl | C(═O)NH | 4 | isothiazol-3,4-diyl | 3 | H | H | H | CH₃ |
| 76B | thiazol-2,4-diyl | C(═O)NH | 2 | isothiazol-3,4-diyl | 3 | H | H | H | CH₃ |
| 77B | thien-2,4-diyl | C(═O)NH | 2 | isothiazol-3,4-diyl | 3 | H | H | H | CH₃ |
| 78B | thien-2,4-diyl | NCH₃C(═O) | 2 | isothiazol-3,4-diyl | 3 | H | H | H | CH₃ |
| 79B | thien-2,4-diyl | NHC(═O) | 2 | isothiazol-3,4-diyl | 3 | H | H | H | CH₃ |
| 80B | oxazol-2,4-diyl | C(═O)NH | 4 | isothiazol-3,4-diyl | 3 | H | H | H | CH₃ |
| 81B | oxazol-2,4-diyl | C(═O)NH | 2 | isothiazol-3,4-diyl | 3 | H | H | H | CH₃ |
| 82B | furan-2,4-diyl | C(═O)NH | 2 | isothiazol-3,4-diyl | 3 | H | H | H | CH₃ |
| 83B | thiazol-2,4-diyl | C(═O)NCH₃ | 2 | isothiazol-3,4-diyl | 3 | H | H | H | Cl |
| 84B | thiazol-2,4-diyl | C(═O)NCH₃ | 4 | isothiazol-3,4-diyl | 3 | H | H | H | Cl |
| 85B | thiazol-2,4-diyl | C(═O)NH | 4 | isothiazol-3,4-diyl | 3 | H | H | H | Cl |
| 86B | thiazol-2,4-diyl | C(═O)NH | 2 | isothiazol-3,4-diyl | 3 | H | H | H | Cl |
| 87B | thien-2,4-diyl | C(═O)NH | 2 | isothiazol-3,4-diyl | 3 | H | H | H | Cl |
| 88B | thien-2,4-diyl | NCH₃C(═O) | 2 | isothiazol-3,4-diyl | 3 | H | H | H | Cl |
| 89B | thiazol-2,4-diyl | C(═O)NCH₃ | 2 | isothiazol-3,4-diyl | 3 | F | F | H | Cl |
| 90B | thiazol-2,4-diyl | C(═O)NCH₃ | 4 | isothiazol-3,4-diyl | 3 | F | F | H | Cl |
| 91B | thiazol-2,4-diyl | C(═O)NH | 4 | isothiazol-3,4-diyl | 3 | F | F | H | Cl |
| 92B | thiazol-2,4-diyl | C(═O)NH | 2 | isothiazol-3,4-diyl | 3 | F | F | H | Cl |
| 93B | oxazol-2,4-diyl | C(═O)NH | 4 | isothiazol-3,4-diyl | 3 | F | F | H | Cl |
| 94B | oxazol-2,4-diyl | C(═O)NH | 2 | isothiazol-3,4-diyl | 3 | F | F | H | Cl |
| 95B | furan-2,4-diyl | C(═O)NH | 2 | isothiazol-3,4-diyl | 3 | F | F | H | Cl |
| 96B | thien-2,4-diyl | C(═O)NH | 2 | isothiazol-3,4-diyl | 3 | F | F | H | Cl |
| 97B | thien-2,4-diyl | NCH₃C(═O) | 2 | isothiazol-3,4-diyl | 3 | F | F | H | Cl |
| 98B | thien-2,4-diyl | NHC(═O) | 2 | isothiazol-3,4-diyl | 3 | F | F | H | Cl |
| 99B | thiazol-2,4-diyl | C(═O)NCH₃ | 4 | 1H-pyrazol-4,5-diyl | 4 | H | F | H | CH₃ |
| 100B | thiazol-2,4-diyl | C(═O)NH | 2 | 1H-pyrazol-4,5-diyl | 4 | H | F | H | CH₃ |
| 101B | oxazol-2,4-diyl | C(═O)NH | 2 | 1H-pyrazol-4,5-diyl | 4 | H | F | H | CH₃ |
| 102B | oxazol-2,4-diyl | C(═O)NH | 4 | 1H-pyrazol-4,5-diyl | 4 | H | F | H | CH₃ |
| 103B | furan-2,4-diyl | C(═O)NH | 2 | 1H-pyrazol-4,5-diyl | 4 | H | F | H | CH₃ |
| 104B | thien-2,4-diyl | C(═O)NH | 2 | 1H-pyrazol-4,5-diyl | 4 | H | F | H | CH₃ |
| 105B | thien-2,4-diyl | NCH₃C(═O) | 2 | 1H-pyrazol-4,5-diyl | 4 | H | F | H | CH₃ |
| 106B | thien-2,4-diyl | NHC(═O) | 2 | 1H-pyrazol-4,5-diyl | 4 | H | F | H | CH₃ |
| 107B | thiazol-2,4-diyl | C(═O)NH | 4 | 1H-pyrazol-4,5-diyl | 4 | H | F | H | CH₃ |
| 108B | thiazol-2,4-diyl | C(═O)NCH₃ | 2 | isothiazol-4,5-diyl | 5 | H | H | H | H |
| 109B | thiazol-2,4-diyl | C(═O)NCH₃ | 4 | isothiazol-4,5-diyl | 5 | H | H | H | H |
| 110B | thiazol-2,4-diyl | C(═O)NH | 4 | isothiazol-4,5-diyl | 5 | H | H | H | H |
| 111B | thiazol-2,4-diyl | C(═O)NH | 2 | isothiazol-4,5-diyl | 5 | H | H | H | H |
| 112B | oxazol-2,4-diyl | C(═O)NH | 2 | isothiazol-4,5-diyl | 5 | H | H | H | H |
| 113B | oxazol-2,4-diyl | C(═O)NH | 4 | isothiazol-4,5-diyl | 5 | H | H | H | H |
| 114B | furan-2,4-diyl | C(═O)NH | 2 | isothiazol-4,5-diyl | 5 | H | H | H | H |
| 115B | thien-2,4-diyl | C(═O)NH | 2 | isothiazol-4,5-diyl | 5 | H | H | H | H |
| 116B | thien-2,4-diyl | NCH₃C(═O) | 2 | isothiazol-4,5-diyl | 5 | H | H | H | H |
| 117B | thien-2,4-diyl | NHC(═O) | 2 | isothiazol-4,5-diyl | 5 | H | H | H | H |
| 118B | thiazol-2,4-diyl | C(═O)NH | 4 | isothiazol-4,5-diyl | 5 | H | H | H | CH₃ |
| 119B | thiazol-2,4-diyl | C(═O)NH | 2 | isothiazol-4,5-diyl | 5 | H | H | H | CH₃ |
| 120B | furan-2,4-diyl | C(═O)NH | 2 | isothiazol-4,5-diyl | 5 | H | H | H | CH₃ |
| 121B | thien-2,4-diyl | NHC(═O) | 2 | isothiazol-4,5-diyl | 5 | H | H | H | CH₃ |
| 122B | thiazol-2,4-diyl | C(═O)NCH₃ | 2 | isothiazol-4,5-diyl | 5 | H | H | H | CH₃ |
| 123B | thiazol-2,4-diyl | C(═O)NCH₃ | 4 | isothiazol-4,5-diyl | 5 | H | H | H | CH₃ |
| 124B | oxazol-2,4-diyl | C(═O)NH | 4 | isothiazol-4,5-diyl | 5 | H | H | H | CH₃ |
| 125B | oxazol-2,4-diyl | C(═O)NH | 2 | isothiazol-4,5-diyl | 5 | H | H | H | CH₃ |
| 126B | thien-2,4-diyl | C(═O)NH | 2 | isothiazol-4,5-diyl | 5 | H | H | H | CH₃ |
| 127B | thien-2,4-diyl | NCH₃C(═O) | 2 | isothiazol-4,5-diyl | 5 | H | H | H | CH₃ |
| 128B | thiazol-2,4-diyl | C(═O)NCH₃ | 2 | isothiazol-4,5-diyl | 5 | H | H | H | Cl |
| 129B | thiazol-2,4-diyl | C(═O)NCH₃ | 4 | isothiazol-4,5-diyl | 5 | H | H | H | Cl |
| 130B | thiazol-2,4-diyl | C(═O)NH | 4 | isothiazol-4,5-diyl | 5 | H | H | H | Cl |
| 131B | thiazol-2,4-diyl | C(═O)NH | 2 | isothiazol-4,5-diyl | 5 | H | H | H | Cl |
| 132B | oxazol-2,4-diyl | C(═O)NH | 2 | isothiazol-4,5-diyl | 5 | H | H | H | Cl |
| 133B | oxazol-2,4-diyl | C(═O)NH | 4 | isothiazol-4,5-diyl | 5 | H | H | H | Cl |
| 134B | furan-2,4-diyl | C(═O)NH | 2 | isothiazol-4,5-diyl | 5 | H | H | H | Cl |
| 135B | thien-2,4-diyl | C(═O)NH | 2 | isothiazol-4,5-diyl | 5 | H | H | H | Cl |
| 136B | thien-2,4-diyl | NCH₃C(═O) | 2 | isothiazol-4,5-diyl | 5 | H | H | H | Cl |
| 137B | thien-2,4-diyl | NHC(═O) | 2 | isothiazol-4,5-diyl | 5 | H | H | H | Cl |
| 138B | thiazol-2,4-diyl | C(═O)NCH₃ | 4 | 1,2,3-triazol-4,5-diyl | 5 | H | H | H | H |
| 139B | thiazol-2,4-diyl | C(═O)NH | 2 | 1,2,3-triazol-4,5-diyl | 5 | H | H | H | H |
| 140B | oxazol-2,4-diyl | C(═O)NH | 2 | 1,2,3-triazol-4,5-diyl | 5 | H | H | H | H |
| 141B | furan-2,4-diyl | C(═O)NH | 2 | 1,2,3-triazol-4,5-diyl | 5 | H | H | H | H |
| 142B | thien-2,4-diyl | C(═O)NH | 2 | 1,2,3-triazol-4,5-diyl | 5 | H | H | H | H |
| 143B | thien-2,4-diyl | NCH₃C(═O) | 2 | 1,2,3-triazol-4,5-diyl | 5 | H | H | H | H |
| 144B | thien-2,4-diyl | NHC(═O) | 2 | 1,2,3-triazol-4,5-diyl | 5 | H | H | H | H |
| 145B | thiazol-2,4-diyl | C(═O)NH | 4 | 1,2,3-triazol-4,5-diyl | 5 | H | H | H | H |
| 146B | oxazol-2,4-diyl | C(═O)NH | 4 | 1,2,3-triazol-4,5-diyl | 5 | H | H | H | H |
| 147B | thiazol-2,4-diyl | C(═O)NCH₃ | 4 | 1,2,3-triazol-4,5-diyl | 5 | H | H | H | CH₃ |
| 148B | thiazol-2,4-diyl | C(═O)NH | 2 | 1,2,3-triazol-4,5-diyl | 5 | H | H | H | CH₃ |
| 149B | oxazol-2,4-diyl | C(═O)NH | 4 | 1,2,3-triazol-4,5-diyl | 5 | H | H | H | CH₃ |
| 150B | furan-2,4-diyl | C(═O)NH | 2 | 1,2,3-triazol-4,5-diyl | 5 | H | H | H | CH₃ |
| 151B | thien-2,4-diyl | C(═O)NH | 2 | 1,2,3-triazol-4,5-diyl | 5 | H | H | H | CH₃ |
| 152B | thien-2,4-diyl | NHC(═O) | 2 | 1,2,3-triazol-4,5-diyl | 5 | H | H | H | CH₃ |
| 153B | thiazol-2,4-diyl | C(═O)NH | 4 | 1,2,3-triazol-4,5-diyl | 5 | H | H | H | CH₃ |
| 154B | oxazol-2,4-diyl | C(═O)NH | 2 | 1,2,3-triazol-4,5-diyl | 5 | H | H | H | CH₃ |
| 155B | thien-2,4-diyl | NCH₃C(═O) | 2 | 1,2,3-triazol-4,5-diyl | 5 | H | H | H | CH₃ |
| 156B | thiazol-2,4-diyl | C(═O)NCH₃ | 4 | 1,2,3-triazol-4,5-diyl | 5 | H | H | H | Cl |
| 157B | thiazol-2,4-diyl | C(═O)NH | 2 | 1,2,3-triazol-4,5-diyl | 5 | H | H | H | Cl |
| 158B | furan-2,4-diyl | C(═O)NH | 2 | 1,2,3-triazol-4,5-diyl | 5 | H | H | H | Cl |
| 159B | thien-2,4-diyl | C(═O)NH | 2 | 1,2,3-triazol-4,5-diyl | 5 | H | H | H | Cl |
| 160B | thien-2,4-diyl | NCH₃C(═O) | 2 | 1,2,3-triazol-4,5-diyl | 5 | H | H | H | Cl |
| 161B | thien-2,4-diyl | NHC(═O) | 2 | 1,2,3-triazol-4,5-diyl | 5 | H | H | H | Cl |
| 162B | thiazol-2,4-diyl | C(═O)NH | 4 | 1,2,3-triazol-4,5-diyl | 5 | H | H | H | Cl |
| 163B | oxazol-2,4-diyl | C(═O)NH | 2 | 1,2,3-triazol-4,5-diyl | 5 | H | H | H | Cl |
| 164B | oxazol-2,4-diyl | C(═O)NH | 4 | 1,2,3-triazol-4,5-diyl | 5 | H | H | H | Cl |
| 165B | thiazol-2,4-diyl | C(═O)NCH₃ | 4 | 1,2,3-triazol-1,5-diyl | 1 | H | H | H | H |
| 166B | thiazol-2,4-diyl | C(═O)NH | 2 | 1,2,3-triazol-1,5-diyl | 1 | H | H | H | H |
| 167B | thien-2,4-diyl | C(═O)NH | 2 | 1,2,3-triazol-1,5-diyl | 1 | H | H | H | H |
| 168B | thien-2,4-diyl | NCH₃C(═O) | 2 | 1,2,3-triazol-1,5-diyl | 1 | H | H | H | H |
| 169B | thien-2,4-diyl | NHC(═O) | 2 | 1,2,3-triazol-1,5-diyl | 1 | H | H | H | H |
| 170B | thiazol-2,4-diyl | C(═O)NH | 4 | 1,2,3-triazol-1,5-diyl | 1 | H | H | H | H |
| 171B | oxazol-2,4-diyl | C(═O)NH | 2 | 1,2,3-triazol-1,5-diyl | 1 | H | H | H | H |
| 172B | oxazol-2,4-diyl | C(═O)NH | 4 | 1,2,3-triazol-1,5-diyl | 1 | H | H | H | H |
| 173B | furan-2,4-diyl | C(═O)NH | 2 | 1,2,3-triazol-1,5-diyl | 1 | H | H | H | H |
| 174B | thiazol-2,4-diyl | C(═O)NCH₃ | 4 | 1,2,3-triazol-1,5-diyl | 1 | H | H | H | CH₃ |
| 175B | thiazol-2,4-diyl | C(═O)NH | 2 | 1,2,3-triazol-1,5-diyl | 1 | H | H | H | CH₃ |
| 176B | furan-2,4-diyl | C(═O)NH | 2 | 1,2,3-triazol-1,5-diyl | 1 | H | H | H | CH₃ |
| 177B | thien-2,4-diyl | C(═O)NH | 2 | 1,2,3-triazol-1,5-diyl | 1 | H | H | H | CH₃ |
| 178B | thien-2,4-diyl | NCH₃C(═O) | 2 | 1,2,3-triazol-1,5-diyl | 1 | H | H | H | CH₃ |
| 179B | thiazol-2,4-diyl | C(═O)NH | 4 | 1,2,3-triazol-1,5-diyl | 1 | H | H | H | CH₃ |
| 180B | oxazol-2,4-diyl | C(═O)NH | 2 | 1,2,3-triazol-1,5-diyl | 1 | H | H | H | CH₃ |
| 181B | oxazol-2,4-diyl | C(═O)NH | 4 | 1,2,3-triazol-1,5-diyl | 1 | H | H | H | CH₃ |
| 182B | thien-2,4-diyl | NHC(═O) | 2 | 1,2,3-triazol-1,5-diyl | 1 | H | H | H | CH₃ |
| 183B | thiazol-2,4-diyl | C(═O)NCH₃ | 4 | 1,2,3-triazol-1,5-diyl | 1 | H | H | H | Cl |
| 184B | thiazol-2,4-diyl | C(═O)NH | 2 | 1,2,3-triazol-1,5-diyl | 1 | H | H | H | Cl |
| 185B | furan-2,4-diyl | C(═O)NH | 2 | 1,2,3-triazol-1,5-diyl | 1 | H | H | H | Cl |
| 186B | thiazol-2,4-diyl | C(═O)NH | 4 | 1,2,3-triazol-1,5-diyl | 1 | H | H | H | Cl |
| 187B | oxazol-2,4-diyl | C(═O)NH | 2 | 1,2,3-triazol-1,5-diyl | 1 | H | H | H | Cl |
| 188B | oxazol-2,4-diyl | C(═O)NH | 4 | 1,2,3-triazol-1,5-diyl | 1 | H | H | H | Cl |
| 189B | thien-2,4-diyl | C(═O)NH | 2 | 1,2,3-triazol-1,5-diyl | 1 | H | H | H | Cl |
| 190B | thien-2,4-diyl | NCH₃C(═O) | 2 | 1,2,3-triazol-1,5-diyl | 1 | H | H | H | Cl |
| 191B | thien-2,4-diyl | NHC(═O) | 2 | 1,2,3-triazol-1,5-diyl | 1 | H | H | H | Cl |
| 192B | thiazol-2,4-diyl | C(═O)NCH₃ | 4 | 1H-pyrazol-4,5-diyl | 4 | H | H | H | H |
| 193B | thiazol-2,4-diyl | C(═O)NH | 2 | 1H-pyrazol-4,5-diyl | 4 | H | H | H | H |
| 194B | furan-2,4-diyl | C(═O)NH | 2 | 1H-pyrazol-4,5-diyl | 4 | H | H | H | H |
| 195B | thien-2,4-diyl | C(═O)NH | 2 | 1H-pyrazol-4,5-diyl | 4 | H | H | H | H |
| 196B | thien-2,4-diyl | NCH₃C(═O) | 2 | 1H-pyrazol-4,5-diyl | 4 | H | H | H | H |
| 197B | thien-2,4-diyl | NHC(═O) | 2 | 1H-pyrazol-4,5-diyl | 4 | H | H | H | H |
| 198B | thiazol-2,4-diyl | C(═O)NH | 4 | 1H-pyrazol-4,5-diyl | 4 | H | H | H | H |
| 199B | oxazol-2,4-diyl | C(═O)NH | 2 | 1H-pyrazol-4,5-diyl | 4 | H | H | H | H |
| 200B | oxazol-2,4-diyl | C(═O)NH | 4 | 1H-pyrazol-4,5-diyl | 4 | H | H | H | H |
| 201B | thiazol-2,4-diyl | C(═O)NCH₃ | 4 | 1H-pyrazol-4,5-diyl | 4 | H | H | H | Cl |
| 202B | thiazol-2,4-diyl | C(═O)NCH₃ | 2 | 3-methyl isooxazol-4,5-diyl | 5 | H | H | H | Cl |
| 203B | thiazol-2,4-diyl | C(═O)NCH₃ | 4 | 3-methyl isooxazol-4,5-diyl | 5 | H | H | H | Cl |
| 204B | thiazol-2,4-diyl | C(═O)NCH₃ | 2 | 3-methyl isooxazol-4,5-diyl | 5 | H | H | H | CH₃ |
| 205B | thiazol-2,4-diyl | C(═O)NCH₃ | 4 | 3-methyl isooxazol-4,5-diyl | 5 | H | H | H | CH₃ |
| 206B | thiazol-2,4-diyl | C(═O)NCH₃ | 2 | isothiazol-3,4-diyl | 3 | H | F | H | Cl |
| 207B | thiazol-2,4-diyl | C(═O)NCH₃ | 4 | isothiazol-3,4-diyl | 3 | H | F | H | Cl |
| 208B | thiazol-2,4-diyl | C(═O)NCH₃ | 2 | isothiazol-3,4-diyl | 3 | F | H | H | Cl |
| 209B | thiazol-2,4-diyl | C(═O)NCH₃ | 4 | isothiazol-3,4-diyl | 3 | F | H | H | Cl |
| 210B | thiazol-2,4-diyl | C(═O)NCH₃ | 2 | 1H-pyrazol-4,5-diyl | 4 | H | H | H | CH₃ |
| 211B | furan-2,4-diyl | C(═O)NCH₃ | 2 | 1H-pyrazol-4,5-diyl | 4 | H | H | H | CH₃ |
| 212B | thien-2,4-diyl | C(═O)NCH₃ | 2 | 1H-pyrazol-4,5-diyl | 4 | H | H | H | CH₃ |
| 213B | thiazol-2,4-diyl | NCH₃C(═O) | 4 | 1H-pyrazol-4,5-diyl | 4 | H | H | H | CH₃ |
| 214B | thiazol-2,4-diyl | NHC(═O) | 4 | 1H-pyrazol-4,5-diyl | 4 | H | H | H | CH₃ |
| 215B | thiazol-2,4-diyl | C(═O)NH | 4 | 1H-pyrazol-4,5-diyl | 4 | H | H | H | CH₃ |
| 216B | oxazol-2,4-diyl | C(═O)NCH₃ | 2 | 1H-pyrazol-4,5-diyl | 4 | H | H | H | CH₃ |
| 217B | oxazol-2,4-diyl | C(═O)NCH₃ | 4 | 1H-pyrazol-4,5-diyl | 4 | H | H | H | CH₃ |
| 218B | thiazol-2,4-diyl | C(═O)NCH₃ | 4 | 1H-pyrazol-4,5-diyl | 4 | H | H | H | CH₃ |
| 219B | furan-2,4-diyl | C(═O)NH | 2 | 1H-pyrazol-4,5-diyl | 4 | H | H | H | Cl |
| 220B | thien-2,4-diyl | NCH₃C(═O) | 2 | 1H-pyrazol-4,5-diyl | 4 | H | H | H | Cl |
| 221B | thien-2,4-diyl | NHC(═O) | 2 | 1H-pyrazol-4,5-diyl | 4 | H | H | H | Cl |
| 222B | oxazol-2,4-diyl | C(═O)NH | 4 | 1H-pyrazol-4,5-diyl | 4 | H | H | H | Cl |
| 223B | thiazol-2,4-diyl | C(═O)NH | 4 | 3-methyl isooxazol-4,5-diyl | 5 | H | H | H | CH₃ |
| 224B | thiazol-2,4-diyl | C(═O)NH | 2 | 3-methyl isooxazol-4,5-diyl | 5 | H | H | H | CH₃ |
| 225B | oxazol-2,4-diyl | C(═O)NH | 2 | 3-methyl isooxazol-4,5-diyl | 5 | H | H | H | CH₃ |
| 226B | oxazol-2,4-diyl | C(═O)NH | 4 | 3-methyl isooxazol-4,5-diyl | 5 | H | H | H | CH₃ |
| 227B | furan-2,4-diyl | C(═O)NH | 2 | 3-methyl isooxazol-4,5-diyl | 5 | H | H | H | CH₃ |
| 228B | thien-2,4-diyl | NHC(═O) | 2 | 3-methyl isooxazol-4,5-diyl | 5 | H | H | H | CH₃ |
| 229B | thien-2,4-diyl | C(═O)NH | 2 | 3-methyl isooxazol-4,5-diyl | 5 | H | H | H | CH₃ |
| 230B | thien-2,4-diyl | NCH₃C(═O) | 2 | 3-methyl isooxazol-4,5-diyl | 5 | H | H | H | CH₃ |
| 231B | thiazol-2,4-diyl | C(═O)NH | 4 | 3-methyl isooxazol-4,5-diyl | 5 | H | H | H | Cl |
| 232B | thiazol-2,4-diyl | C(═O)NH | 2 | 3-methyl isooxazol-4,5-diyl | 5 | H | H | H | Cl |
| 233B | thien-2,4-diyl | C(═O)NH | 2 | 3-methyl isooxazol-4,5-diyl | 5 | H | H | H | Cl |
| 234B | thien-2,4-diyl | NCH₃C(═O) | 2 | 3-methyl isooxazol-4,5-diyl | 5 | H | H | H | Cl |
| 235B | thien-2,4-diyl | NHC(═O) | 2 | 3-methyl isooxazol-4,5-diyl | 5 | H | H | H | Cl |
| 236B | oxazol-2,4-diyl | C(═O)NH | 4 | 3-methyl isooxazol-4,5-diyl | 5 | H | H | H | Cl |
| 237B | oxazol-2,4-diyl | C(═O)NH | 2 | 3-methyl isooxazol-4,5-diyl | 5 | H | H | H | Cl |
| 238B | furan-2,4-diyl | C(═O)NH | 2 | 3-methyl isooxazol-4,5-diyl | 5 | H | H | H | Cl |
| 239B | thiazol-2,4-diyl | C(═O)NH | 4 | isothiazol-3,4-diyl | 3 | H | H | H | Cl |
| 240B | thiazol-2,4-diyl | C(═O)NH | 2 | isothiazol-3,4-diyl | 3 | H | H | H | Cl |
| 241B | thiazol-2,4-diyl | C(═O)NH | 4 | isothiazol-3,4-diyl | 3 | F | H | H | Cl |
| 242B | thiazol-2,4-diyl | C(═O)NH | 2 | isothiazol-3,4-diyl | 3 | F | H | H | Cl |
| 243B | oxazol-2,4-diyl | C(═O)NH | 4 | isothiazol-3,4-diyl | 3 | H | H | H | Cl |
| 244B | oxazol-2,4-diyl | C(═O)NH | 2 | isothiazol-3,4-diyl | 3 | H | H | H | Cl |
| 245B | oxazol-2,4-diyl | C(═O)NH | 4 | isothiazol-3,4-diyl | 3 | H | F | H | Cl |
| 246B | oxazol-2,4-diyl | C(═O)NH | 2 | isothiazol-3,4-diyl | 3 | H | F | H | Cl |
| 247B | oxazol-2,4-diyl | C(═O)NH | 4 | isothiazol-3,4-diyl | 3 | F | H | H | Cl |
| 248B | oxazol-2,4-diyl | C(═O)NH | 2 | isothiazol-3,4-diyl | 3 | F | H | H | Cl |
| 249B | oxazol-2,4-diyl | NCH₃C(═O) | 4 | 1H-pyrazol-4,5-diyl | 4 | H | H | H | CH₃ |
| 250B | oxazol-2,4-diyl | NHC(═O) | 4 | 1H-pyrazol-4,5-diyl | 4 | H | H | H | CH₃ |
| 251B | furan-2,4-diyl | C(═O)NH | 2 | isothiazol-3,4-diyl | 3 | H | H | H | Cl |
| 252B | furan-2,4-diyl | C(═O)NH | 2 | isothiazol-3,4-diyl | 3 | H | F | H | Cl |
| 253B | furan-2,4-diyl | C(═O)NH | 2 | isothiazol-3,4-diyl | 3 | F | H | H | Cl |
| 254B | furan-2,4-diyl | NCH₃C(═O) | 2 | 1H-pyrazol-4,5-diyl | 4 | H | H | H | CH₃ |
| 255B | furan-2,4-diyl | NHC(═O) | 2 | 1H-pyrazol-4,5-diyl | 4 | H | H | H | CH₃ |
| 256B | thien-2,4-diyl | C(═O)NH | 2 | isothiazol-3,4-diyl | 3 | H | F | H | Cl |
| 257B | thien-2,4-diyl | C(═O)NH | 2 | isothiazol-3,4-diyl | 3 | F | H | H | Cl |
| 258B | thien-2,4-diyl | NCH₃C(═O) | 2 | isothiazol-3,4-diyl | 3 | H | F | H | Cl |
| 259B | thien-2,4-diyl | NCH₃C(═O) | 2 | isothiazol-3,4-diyl | 3 | F | H | H | Cl |
| 260B | thien-2,4-diyl | NHC(═O) | 2 | 1H-pyrazol-4,5-diyl | 4 | H | H | H | CH₃ |
| 261B | thien-2,4-diyl | NCH₃C(═O) | 2 | 1H-pyrazol-4,5-diyl | 4 | H | H | H | CH₃ |
| 262B | thien-2,4-diyl | NHC(═O) | 2 | isothiazol-3,4-diyl | 3 | H | H | H | Cl |
| 263B | thien-2,4-diyl | NHC(═O) | 2 | isothiazol-3,4-diyl | 3 | H | F | H | Cl |
| 264B | thien-2,4-diyl | NHC(═O) | 2 | isothiazol-3,4-diyl | 3 | F | H | H | Cl |
| 265B | pyridin-2-one-1,4-diyl | n/a | 4 | 1,2,3-triazol-4,5-diyl | 5 | H | H | H | CH₃ |

Note:
Column * indicates ring C's point of connection to the adjacent optinally substituted phen-1,4-ylene; Column # indicates ring A's point of connection to the adjacent cyclopropyl.

In some embodiments, compounds of Formula (III) are also represented by Formula (III-C) and are selected from the following compounds as listed in Table 3C.

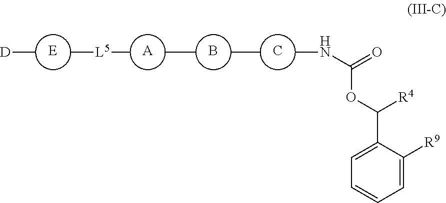

TABLE 3C

| Cmpd # III- | 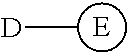 | L⁵ | 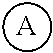 | 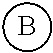 | 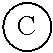 | * | R⁴ | R⁹ |
|---|---|---|---|---|---|---|---|---|
| 1C | 3-carboxy-pyridin-2-yl | —NH— | pyridin-2-one-1,4-diyl | 1,4-phenylene | isothiazol-3,4-diyl | 3 | CH₃ | H |
| 2C | 2-carboxy-phenyl | —S(O)₂— | 1,4-phenylene | pyridin-2-one-1,4-diyl | 1H-pyrazol-4,5-diyl | 4 | CH₃ | H |
| 3C | 2-carboxy-phenyl | —NH— | 1,4-phenylene | pyridin-2-one-1,4-diyl | 1H-pyrazol-4,5-diyl | 4 | CH₃ | H |
| 4C | 2-carboxy-phenyl | —S(O)₂— | 1,4-phenylene | pyridin-2-one-1,4-diyl | 1H-pyrazol-4,5-diyl | 4 | H | H |
| 5C | 2-carboxy-phenyl | —NH— | 1,4-phenylene | pyridin-2-one-1,4-diyl | 1H-pyrazol-4,5-diyl | 4 | H | H |
| 6C | 3-carboxy-pyridin-2-yl | —NH— | pyridin-2-one-1,4-diyl | 1,4-phenylene | 5-methyl isothiazol-3,4-diyl | 3 | H | H |
| 7C | 2-carboxy-phenyl | —S(O)₂— | 1,4-phenylene | pyridin-2-one-1,4-diyl | 1-methyl-pyrazol-4,5-diyl | 4 | H | CH₃ |
| 8C | 2-carboxy-phenyl | —NH— | 1,4-phenylene | pyridin-2-one-1,4-diyl | 1-methyl-pyrazol-4,5-diyl | 4 | H | CH₃ |
| 9C | 2-carboxy-phenyl | —S(O)₂— | 1,4-phenylene | pyridin-2-one-1,4-diyl | 3-methyl isooxazol-4,5-diyl | 5 | CH₃ | H |
| 10C | 2-carboxy-phenyl | —NH— | 1,4-phenylene | pyridin-2-one-1,4-diyl | 3-methyl isooxazol-4,5-diyl | 5 | CH₃ | H |
| 11C | 3-carboxy-pyridin-2-yl | —NH— | pyridin-2-one-1,4-diyl | 1,4-phenylene | 3-methyl isooxazol-4,5-diyl | 5 | CH₃ | H |
| 12C | 3-carboxy-pyridin-2-yl | —NH— | pyridin-2-one-1,4-diyl | 1,4-phenylene | isothiazol-3,4-diyl | 3 | H | H |
| 13C | 2-carboxy-phenyl | —S(O)₂— | 1,4-phenylene | pyridin-2-one-1,4-diyl | isooxazol-3,4-diyl | 3 | H | H |
| 14C | 2-carboxy-phenyl | —NH— | 1,4-phenylene | pyridin-2-one-1,4-diyl | isooxazol-3,4-diyl | 3 | H | H |
| 15C | 3-carboxy-pyridin-2-yl | —NH— | pyridin-2-one-1,4-diyl | 1,4-phenylene | isooxazol-3,4-diyl | 3 | H | H |
| 16C | 2-carboxy-phenyl | —NH— | pyridin-2-one-1,4-diyl | 1,4-phenylene | isothiazol-3,4-diyl | 3 | H | H |
| 17C | 3-carboxy-pyridin-2-yl | —NH— | pyridin-2-one-1,4-diyl | 1,4-phenylene | isooxazol-3,4-diyl | 3 | H | CH₃ |
| 18C | 2-carboxy-phenyl | —S(O)₂— | 1,4-phenylene | pyridin-2-one-1,4-diyl | isooxazol-3,4-diyl | 3 | H | CH₃ |
| 19C | 2-carboxy-phenyl | —NH— | 1,4-phenylene | pyridin-2-one-1,4-diyl | isooxazol-3,4-diyl | 3 | H | CH₃ |
| 20C | 2-carboxy-phenyl | —S(O)₂— | 1,4-phenylene | pyridin-2-one-1,4-diyl | isooxazol-3,4-diyl | 3 | H | Cl |
| 21C | 2-carboxy-phenyl | —NH— | 1,4-phenylene | pyridin-2-one-1,4-diyl | isooxazol-3,4-diyl | 3 | H | Cl |
| 22C | 3-carboxy-pyridin-2-yl | —NH— | pyridin-2-one-1,4-diyl | 1,4-phenylene | isooxazol-3,4-diyl | 3 | H | Cl |
| 23C | 3-carboxy-pyridin-2-yl | —NH— | pyridin-2-one-1,4-diyl | 1,4-phenylene | isothiazol-3,4-diyl | 3 | H | CH₃ |
| 24C | 2-carboxy-phenyl | —S(O)₂— | 1,4-phenylene | pyridin-2-one-1,4-diyl | isothiazol-3,4-diyl | 3 | H | H |
| 25C | 2-carboxy-phenyl | —NH— | 1,4-phenylene | pyridin-2-one-1,4-diyl | isothiazol-3,4-diyl | 3 | H | H |
| 26C | 3-carboxy-pyridin-2-yl | —NH— | pyridin-2-one-1,4-diyl | 1,4-phenylene | 1H-pyrazol-4,5-diyl | 4 | H | CH₃ |
| 27C | 3-carboxy-pyridin-2-yl | —NH— | pyridin-2-one-1,4-diyl | 1,4-phenylene | 1H-pyrazol-4,5-diyl | 4 | H | Cl |
| 28C | 3-carboxy-pyridin-2-yl | —NH— | pyridin-2-one-1,4-diyl | 1,4-phenylene | 3-methyl isooxazol-4,5-diyl | 5 | H | CH₃ |
| 29C | 3-carboxy-pyridin-2-yl | —NH— | pyridin-2-one-1,4-diyl | 1,4-phenylene | isothiazol-3,4-diyl | 3 | H | Cl |
| 30C | 3-carboxy-pyridin-2-yl | —NH— | pyridin-2-one-1,4-diyl | 1,4-phenylene | isothiazol-3,4-diyl | 5 | H | H |
| 31C | 3-carboxy-pyridin-2-yl | —NH— | pyridin-2-one-1,4-diyl | 1,4-phenylene | isothiazol-3,4-diyl | 5 | H | CH₃ |
| 32C | 3-carboxy-pyridin-2-yl | —NH— | pyridin-2-one-1,4-diyl | 1,4-phenylene | isothiazol-3,4-diyl | 5 | H | Cl |
| 33C | 3-carboxy-pyridin-2-yl | —NH— | pyridin-2-one-1,4-diyl | 1,4-phenylene | 1,2,3-triazol-4,5-diyl | 5 | H | H |
| 34C | 3-carboxy-pyridin-2-yl | —NH— | pyridin-2-one-1,4-diyl | 1,4-phenylene | 1,2,3-triazol-4,5-diyl | 5 | H | CH₃ |
| 35C | 3-carboxy-pyridin-2-yl | —NH— | pyridin-2-one-1,4-diyl | 1,4-phenylene | 1,2,3-triazol-4,5-diyl | 5 | H | Cl |
| 36C | 3-carboxy-pyridin-2-yl | —NH— | pyridin-2-one-1,4-diyl | 1,4-phenylene | 1,2,3-triazol-4,5-diyl | 1 | H | H |
| 37C | 3-carboxy-pyridin-2-yl | —NH— | pyridin-2-one-1,4-diyl | 1,4-phenylene | 1,2,3-triazol-4,5-diyl | 1 | H | CH₃ |
| 38C | 3-carboxy-pyridin-2-yl | —NH— | pyridin-2-one-1,4-diyl | 1,4-phenylene | 1,2,3-triazol-4,5-diyl | 1 | H | Cl |
| 39C | 3-carboxy-pyridin-2-yl | —NH— | pyridin-2-one-1,4-diyl | 1,4-phenylene | 1H-pyrazol-4,5-diyl | 4 | H | H |
| 40C | 3-carboxy-pyridin-2-yl | —NH— | pyridin-2-one-1,4-diyl | 1,4-phenylene | 3-methyl isooxazol-4,5-diyl | 5 | H | Cl |
| 41C | 2-carboxy-phenyl | —S— | pyridin-2-one-1,4-diyl | 1,4-phenylene | isothiazol-3,4-diyl | 3 | H | H |
| 42C | 3-carboxy-pyridin-2-yl | —NH— | 1,4-phenylene | pyridin-2-one-1,4-diyl | 1H-pyrazol-4,5-diyl | 4 | H | CH₃ |
| 43C | 4-carboxy-pyridin-3-yl | —NH— | pyridin-2-one-1,4-diyl | 1,4-phenylene | isothiazol-3,4-diyl | 3 | H | H |
| 44C | 3-carboxy-pyridin-4-yl | —NH— | pyridin-2-one-1,4-diyl | 1,4-phenylene | isothiazol-3,4-diyl | 3 | H | H |
| 45C | 2-carboxy-phenyl | —S(O)₂— | 1,4-phenylene | pyridin-2-one-1,4-diyl | isothiazol-3,4-diyl | 3 | H | CH₃ |
| 46C | 2-carboxy-phenyl | —NH— | 1,4-phenylene | pyridin-2-one-1,4-diyl | isothiazol-3,4-diyl | 3 | H | CH₃ |
| 47C | 2-carboxy-phenyl | —S(O)₂— | 1,4-phenylene | pyridin-2-one-1,4-diyl | isothiazol-3,4-diyl | 3 | H | Cl |
| 48C | 2-carboxy-phenyl | —NH— | 1,4-phenylene | pyridin-2-one-1,4-diyl | isothiazol-3,4-diyl | 3 | H | Cl |
| 49C | 2-carboxy-phenyl | —S(O)₂— | 1,4-phenylene | pyridin-2-one-1,4-diyl | isothiazol-4,5-diyl | 5 | H | H |
| 50C | 2-carboxy-phenyl | —NH— | 1,4-phenylene | pyridin-2-one-1,4-diyl | isothiazol-4,5-diyl | 5 | H | H |
| 51C | 2-carboxy-phenyl | —S(O)₂— | 1,4-phenylene | pyridin-2-one-1,4-diyl | isothiazol-4,5-diyl | 5 | H | CH₃ |
| 52C | 2-carboxy-phenyl | —NH— | 1,4-phenylene | pyridin-2-one-1,4-diyl | isothiazol-4,5-diyl | 5 | H | CH₃ |
| 53C | 2-carboxy-phenyl | —S(O)₂— | 1,4-phenylene | pyridin-2-one-1,4-diyl | isothiazol-4,5-diyl | 5 | H | Cl |
| 54C | 2-carboxy-phenyl | —NH— | 1,4-phenylene | pyridin-2-one-1,4-diyl | isothiazol-4,5-diyl | 5 | H | Cl |
| 55C | 2-carboxy-phenyl | —S(O)₂— | 1,4-phenylene | pyridin-2-one-1,4-diyl | 1,2,3-triazol-4,5-diyl | 5 | H | H |
| 56C | 2-carboxy-phenyl | —NH— | 1,4-phenylene | pyridin-2-one-1,4-diyl | 1,2,3-triazol-4,5-diyl | 5 | H | H |
| 57C | 2-carboxy-phenyl | —S(O)₂— | 1,4-phenylene | pyridin-2-one-1,4-diyl | 1,2,3-triazol-4,5-diyl | 5 | H | CH₃ |
| 58C | 2-carboxy-phenyl | —NH— | 1,4-phenylene | pyridin-2-one-1,4-diyl | 1,2,3-triazol-4,5-diyl | 5 | H | CH₃ |
| 59C | 2-carboxy-phenyl | —S(O)₂— | 1,4-phenylene | pyridin-2-one-1,4-diyl | 1,2,3-triazol-4,5-diyl | 5 | H | Cl |
| 60C | 2-carboxy-phenyl | —NH— | 1,4-phenylene | pyridin-2-one-1,4-diyl | 1,2,3-triazol-4,5-diyl | 5 | H | Cl |
| 61C | 2-carboxy-phenyl | —S(O)₂— | 1,4-phenylene | pyridin-2-one-1,4-diyl | 1,2,3-triazol-1,5-diyl | 1 | H | H |
| 62C | 2-carboxy-phenyl | —NH— | 1,4-phenylene | pyridin-2-one-1,4-diyl | 1,2,3-triazol-1,5-diyl | 1 | H | H |
| 63C | 2-carboxy-phenyl | —S(O)₂— | 1,4-phenylene | pyridin-2-one-1,4-diyl | 1,2,3-triazol-1,5-diyl | 1 | H | CH₃ |
| 64C | 2-carboxy-phenyl | —NH— | 1,4-phenylene | pyridin-2-one-1,4-diyl | 1,2,3-triazol-1,5-diyl | 1 | H | CH₃ |
| 65C | 2-carboxy-phenyl | —S(O)₂— | 1,4-phenylene | pyridin-2-one-1,4-diyl | 1,2,3-triazol-1,5-diyl | 1 | H | Cl |
| 66C | 2-carboxy-phenyl | —NH— | 1,4-phenylene | pyridin-2-one-1,4-diyl | 1,2,3-triazol-1,5-diyl | 1 | H | Cl |
| 67C | 2-carboxy-phenyl | —S— | 1,4-phenylene | pyridin-2-one-1,4-diyl | 1H-pyrazol-4,5-diyl | 4 | H | CH₃ |
| 68C | 2-carboxy-phenyl | —S(O)₂— | 1,4-phenylene | pyridin-2-one-1,4-diyl | 1H-pyrazol-4,5-diyl | 4 | H | Cl |
| 69C | 2-carboxy-phenyl | —NH— | 1,4-phenylene | pyridin-2-one-1,4-diyl | 1H-pyrazol-4,5-diyl | 4 | H | Cl |
| 70C | 2-carboxy-phenyl | —S(O)₂— | 1,4-phenylene | pyridin-2-one-1,4-diyl | 3-methyl isooxazol-4,5-diyl | 5 | H | CH₃ |
| 71C | 2-carboxy-phenyl | —NH— | 1,4-phenylene | pyridin-2-one-1,4-diyl | 3-methyl isooxazol-4,5-diyl | 5 | H | CH₃ |
| 72C | 2-carboxy-phenyl | —S(O)₂— | 1,4-phenylene | pyridin-2-one-1,4-diyl | 3-methyl isooxazol-4,5-diyl | 5 | H | Cl |
| 73C | 2-carboxy-phenyl | —NH— | 1,4-phenylene | pyridin-2-one-1,4-diyl | 3-methyl isooxazol-4,5-diyl | 5 | H | Cl |
| 74C | 6-carboxy-benzo[d]thiazol-5-yl | —NH— | pyridin-2-one-1,4-diyl | 1,4-phenylene | isothiazol-3,4-diyl | 3 | H | H |
| 75C | 6-carboxy-benzo[d]thiazol-5-yl | —S(O)₂— | 1,4-phenylene | pyridin-2-one-1,4-diyl | 1H-pyrazol-4,5-diyl | 4 | H | CH₃ |
| 76C | 6-carboxy-benzo[d]thiazol-5-yl | —NH— | 1,4-phenylene | pyridin-2-one-1,4-diyl | 1H-pyrazol-4,5-diyl | 4 | H | CH₃ |
| 77C | 2-carboxy-phenyl | —S(O)₂— | pyridin-2-one-1,4-diyl | 1,4-phenylene | isothiazol-3,4-diyl | 3 | H | H |
| 78C | 4-carboxy-benzo[d]thiazol-5-yl | —NH— | pyridin-2-one-1,4-diyl | 1,4-phenylene | isothiazol-3,4-diyl | 3 | H | H |
| 79C | 5-carboxy-benzo[d]oxazol-4-yl | —NH— | pyridin-2-one-1,4-diyl | 1,4-phenylene | isothiazol-3,4-diyl | 3 | H | H |
| 80C | 5-carboxy-benzo[d]oxazol-4-yl | —NH— | pyridin-2-one-1,4-diyl | 1,4-phenylene | isothiazol-3,4-diyl | 3 | H | H |
| 81C | 6-carboxy-benzo[d]oxazol-5-yl | —NH— | pyridin-2-one-1,4-diyl | 1,4-phenylene | isothiazol-3,4-diyl | 3 | H | H |
| 82C | 4-carboxy-benzo[d]oxazol-5-yl | —NH— | pyridin-2-one-1,4-diyl | 1,4-phenylene | isothiazol-3,4-diyl | 3 | H | H |
| 83C | 4-carboxy-benzo[d]thiazol-5-yl | —S(O)₂— | 1,4-phenylene | pyridin-2-one-1,4-diyl | 1H-pyrazol-4,5-diyl | 4 | H | CH₃ |
| 84C | 4-carboxy-benzo[d]thiazol-5-yl | —NH— | 1,4-phenylene | pyridin-2-one-1,4-diyl | 1H-pyrazol-4,5-diyl | 4 | H | CH₃ |
| 85C | 6-carboxy-benzo[d]oxazol-5-yl | —S(O)₂— | 1,4-phenylene | pyridin-2-one-1,4-diyl | 1H-pyrazol-4,5-diyl | 4 | H | CH₃ |
| 86C | 6-carboxy-benzo[d]oxazol-5-yl | —NH— | 1,4-phenylene | pyridin-2-one-1,4-diyl | 1H-pyrazol-4,5-diyl | 4 | H | CH₃ |
| 87C | 2-carboxy-phenyl | —S(O)₂— | 1,4-phenylene | pyridin-2-one-1,4-diyl | 1H-pyrazol-4,5-diyl | 4 | H | CH₃ |
| 88C | 5-carboxy-benzo[d]oxazol-4-yl | —S(O)₂— | 1,4-phenylene | pyridin-2-one-1,4-diyl | 1H-pyrazol-4,5-diyl | 4 | H | CH₃ |
| 89C | 5-carboxy-benzo[d]oxazol-4-yl | —NH— | 1,4-phenylene | pyridin-2-one-1,4-diyl | 1H-pyrazol-4,5-diyl | 4 | H | CH₃ |
| 90C | 5-carboxy-benzo[d]thiazol-4-yl | —S(O)₂— | 1,4-phenylene | pyridin-2-one-1,4-diyl | 1H-pyrazol-4,5-diyl | 4 | H | CH₃ |
| 91C | 5-carboxy-benzo[d]thiazol-4-yl | —NH— | 1,4-phenylene | pyridin-2-one-1,4-diyl | 1H-pyrazol-4,5-diyl | 4 | H | CH₃ |
| 92C | 4-carboxy-benzo[d]oxazol-5-yl | —S(O)₂— | 1,4-phenylene | pyridin-2-one-1,4-diyl | 1H-pyrazol-4,5-diyl | 4 | H | CH₃ |
| 93C | 4-carboxy-benzo[d]oxazol-5-yl | —NH— | 1,4-phenylene | pyridin-2-one-1,4-diyl | 1H-pyrazol-4,5-diyl | 4 | H | CH₃ |

Note:
Column * indicates ring C's point of connection to the adjacent ring B.

In some embodiments, compounds of Formula (IV) are selected from the following compounds as listed in Table 4.

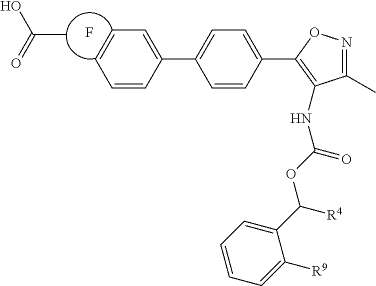

TABLE 4

| Cmpd # IV- | 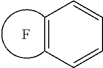 | * | R⁴ | R⁹ |
|---|---|---|---|---|
| 1 | 1,2,3,4-tetrahydroquinolin-4,7-diyl | 4 | CH₃ | H |
| 2 | chroman-4,7-diyl | 4 | CH₃ | H |
| 3 | 1,2,3,4-tetrahydroisoquinolin-4,7-diyl | 4 | CH₃ | H |
| 4 | isochroman-4,7-diyl | 4 | CH₃ | H |
| 5 | 1,2,3,4-tetrahydroisoquinolin-1,6-diyl | 1 | CH₃ | H |
| 6 | isochroman-1,6-diyl | 1 | H | H |
| 7 | 2-oxo-1,2,3,4-tetrahydroquinolin-4,7-diyl | 4 | CH₃ | Cl |
| 8 | 3-oxo-1,2,3,4-tetrahydroisoquinolin-4,7-diyl | 4 | CH₃ | CH₃ |
| 9 | 3-oxo-1,2,3,4-tetrahydroisoquinolin-1,6-diyl | 1 | CH₃ | H |
| 10 | indolin-3,6-diyl | 3 | CH₃ | H |
| 11 | dihydrobenzofuran-3,6-diyl | 3 | CH₃ | H |
| 12 | isoindolin-3,6-diyl | 3 | CH₃ | H |
| 13 | dihydroisobenzofuran-3,6-diyl | 3 | CH₃ | H |
| 14 | 2-oxo-indolin-3,6-diyl | 3 | CH₃ | H |

Note:

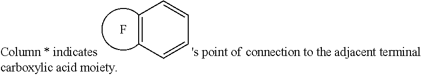

In some embodiments, compounds of Formula (V) are selected from the following compounds as listed in Table 5.

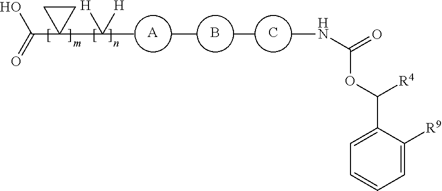

TABLE 5

| Cmpd # V- | m | n | 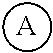 | + | 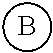 | # | 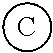 | * | R⁴ | R⁹ |
|---|---|---|---|---|---|---|---|---|---|---|
| 1 | 0 | 0 | 1H-indol-2,6-diyl | 6 | ethyne-1,2-diyl | 2 | 3-methyl-isooxazol-4,5-diyl | 5 | CH₃ | H |
| 2 | 1 | 0 | 1H-indol-2,6-diyl | 6 | ethyne-1,2-diyl | 2 | 3-methyl-isooxazol-4,5-diyl | 5 | CH₃ | H |
| 3 | 0 | 0 | 1H-indol-2,6-diyl | 7 | ethyne-1,2-diyl | 2 | 3-methyl-isooxazol-4,5-diyl | 5 | CH₃ | H |
| 4 | 1 | 0 | 1H-indol-2,6-diyl | 7 | ethyne-1,2-diyl | 2 | 3-methyl-isooxazol-4,5-diyl | 5 | CH₃ | H |
| 5 | 0 | 0 | ethyne-1,2-diyl | 1 | 1H-indol-2,6-diyl | 6 | 3-methyl-isooxazol-4,5-diyl | 5 | CH₃ | H |
| 6 | 1 | 0 | ethyne-1,2-diyl | 1 | 1H-indol-2,6-diyl | 6 | 3-methyl-isooxazol-4,5-diyl | 5 | CH₃ | H |
| 7 | 0 | 0 | 1,2,3,4-tetrahydronaphthalene-2,7-diyl | 2 | ethyne-1,2-diyl | 2 | 3-methyl-isooxazol-4,5-diyl | 5 | CH₃ | H |
| 8 | 0 | 0 | 1,2,3,4-tetrahydronaphthalene-1,7-diyl | 1 | ethyne-1,2-diyl | 2 | 3-methyl-isooxazol-4,5-diyl | 5 | CH₃ | H |
| 9 | 1 | 0 | 1,2,3,4-tetrahydronaphthalene-1,7-diyl | 1 | ethyne-1,2-diyl | 2 | 3-methyl-isooxazol-4,5-diyl | 5 | CH₃ | H |
| 10 | 0 | 0 | 1,2,3,4-tetrahydronaphthalene-2,7-diyl | 2 | ethyne-1,2-diyl | 2 | 3-methyl-isooxazol-4,5-diyl | 5 | CH₃ | H |
| 11 | 0 | 0 | 1,2,3,4-tetrahydronaphthalene-1,7-diyl | 1 | ethyne-1,2-diyl | 2 | 3-methyl-isooxazol-4,5-diyl | 5 | CH₃ | H |
| 12 | 1 | 0 | 1,2,3,4-tetrahydronaphthalene-1,7-diyl | 1 | ethyne-1,2-diyl | 2 | 3-methyl-isooxazol-4,5-diyl | 5 | CH₃ | H |
| 13 | 1 | 0 | 1H-benzo[d]imidazol-2,5-diyl | 2 | ethyne-1,2-diyl | 2 | 3-methyl-isooxazol-4,5-diyl | 5 | CH₃ | H |
| 14 | 1 | 0 | benzo[d]thiazol-2,6-diyl | 2 | ethyne-1,2-diyl | 2 | 3-methyl-isooxazol-4,5-diyl | 5 | CH₃ | H |
| 15 | 1 | 0 | benzo[d]thiazol-2,6-diyl | 2 | ethyne-1,2-diyl | 2 | 3-methyl-isooxazol-4,5-diyl | 5 | CH₃ | H |
| 16 | 0 | 0 | 2,3-dihydro-1H-indene-1,5-diyl | 1 | ethyne-1,2-diyl | 2 | 3-methyl-isooxazol-4,5-diyl | 5 | CH₃ | H |
| 17 | 0 | 0 | indolin-3,6-diyl | 3 | ethyne-1,2-diyl | 2 | 3-methyl-isooxazol-4,5-diyl | 5 | CH₃ | H |
| 18 | 0 | 0 | 2.3-dihydrobenzofuran-3,6-diyl | 3 | ethyne-1,2-diyl | 2 | 3-methyl-isooxazol-4,5-diyl | 5 | CH₃ | H |
| 19 | 0 | 0 | isoindolin-1,5-diyl | 1 | ethyne-1,2-diyl | 2 | 3-methyl-isooxazol-4,5-diyl | 5 | CH₃ | H |
| 20 | 0 | 0 | 1,3-dihydroisobenzo-furan-1,5-diyl | 1 | ethyne-1,2-diyl | 2 | 3-methyl-isooxazol-4,5-diyl | 5 | CH₃ | H |
| 21 | 0 | 0 | 2-oxo-indolin-3,6-diyl | 3 | ethyne-1,2-diyl | 2 | 3-methyl-isooxazol-4,5-diyl | 5 | CH₃ | H |
| 22 | 0 | 0 | 1H-indol-3,6-diyl | 3 | ethyne-1,2-diyl | 2 | 3-methyl-isooxazol-4,5-diyl | 5 | CH₃ | H |
| 23 | 0 | 0 | benzofuran-3,6-diyl | 3 | ethyne-1,2-diyl | 2 | 3-methyl-isooxazol-4,5-diyl | 5 | CH₃ | H |
| 24 | 0 | 1 | 1H-indol-3,6-diyl | 3 | ethyne-1,2-diyl | 2 | 3-methyl-isooxazol-4,5-diyl | 5 | CH₃ | H |
| 25 | 0 | 1 | benzofuran-3,6-diyl | 3 | ethyne-1,2-diyl | 2 | 3-methyl-isooxazol-4,5-diyl | 5 | CH₃ | H |
| 26 | 0 | 0 | benzo[b]thiophene-3,6-diyl | 3 | ethyne-1,2-diyl | 2 | 3-methyl-isooxazol-4,5-diyl | 5 | CH₃ | H |
| 27 | 1 | 0 | 1H-indol-3,6-diyl | 3 | ethyne-1,2-diyl | 2 | 3-methyl-isooxazol-4,5-diyl | 5 | CH₃ | H |
| 28 | 1 | 0 | benzofuran-3,6-diyl | 3 | ethyne-1,2-diyl | 2 | 3-methyl-isooxazol-4,5-diyl | 5 | CH₃ | H |
| 29 | 0 | 0 | benzo[b]thiophene-2,6-diyl | 6 | ethyne-1,2-diyl | 2 | 3-methyl-isooxazol-4,5-diyl | 5 | CH₃ | H |
| 30 | 1 | 0 | benzo[b]thiophene-2,6-diyl | 6 | ethyne-1,2-diyl | 2 | 3-methyl-isooxazol-4,5-diyl | 5 | CH₃ | H |
| 31 | 0 | 0 | 1H-indol-2,6-diyl | 2 | ethyne-1,2-diyl | 2 | 3-methyl-isooxazol-4,5-diyl | 5 | CH₃ | H |
| 32 | 0 | 0 | benzofuran-2,6-diyl | 2 | ethyne-1,2-diyl | 2 | 3-methyl-isooxazol-4,5-diyl | 5 | CH₃ | H |
| 33 | 0 | 0 | benzo[b]thiophene-2,6-diyl | 2 | ethyne-1,2-diyl | 2 | 3-methyl-isooxazol-4,5-diyl | 5 | CH₃ | H |
| 34 | 0 | 0 | 1H-indol-2,6-diyl | 2 | ethyne-1,2-diyl | 2 | 3-methyl-isooxazol-4,5-diyl | 5 | H | H |
| 35 | 0 | 0 | benzofuran-2,6-diyl | 2 | ethyne-1,2-diyl | 2 | 3-methyl-isooxazol-4,5-diyl | 5 | CH₃ | Cl |
| 36 | 0 | 1 | 1H-indol-2,6-diyl | 2 | ethyne-1,2-diyl | 2 | 3-methyl-isooxazol-4,5-diyl | 5 | CH₃ | H |
| 37 | 0 | 1 | benzofuran-2,6-diyl | 2 | ethyne-1,2-diyl | 2 | 3-methyl-isooxazol-4,5-diyl | 5 | CH₃ | H |
| 38 | 0 | 1 | benzo[b]thiophene-2,6-diyl | 2 | ethyne-1,2-diyl | 2 | 3-methyl-isooxazol-4,5-diyl | 5 | CH₃ | H |
| 39 | 0 | 1 | 1H-indol-2,6-diyl | 2 | ethyne-1,2-diyl | 2 | 3-methyl-isooxazol-4,5-diyl | 5 | H | H |
| 40 | 0 | 1 | benzofuran-2,6-diyl | 2 | ethyne-1,2-diyl | 2 | 3-methyl-isooxazol-4,5-diyl | 5 | CH₃ | CH₃ |
| 41 | 1 | 0 | 1H-indol-2,6-diyl | 2 | ethyne-1,2-diyl | 2 | 3-methyl-isooxazol-4,5-diyl | 5 | CH₃ | H |
| 42 | 1 | 0 | benzofuran-2,6-diyl | 2 | ethyne-1,2-diyl | 2 | 3-methyl-isooxazol-4,5-diyl | 5 | CH₃ | H |
| 43 | 1 | 0 | benzo[b]thiophene-2,6-diyl | 2 | ethyne-1,2-diyl | 2 | 3-methyl-isooxazol-4,5-diyl | 5 | CH₃ | H |
| 44 | 1 | 0 | 1H-indol-2,6-diyl | 2 | ethyne-1,2-diyl | 2 | 3-methyl-isooxazol-4,5-diyl | 5 | H | H |
| 45 | 1 | 0 | benzofuran-2,6-diyl | 2 | ethyne-1,2-diyl | 2 | 3-methyl-isooxazol-4,5-diyl | 5 | CH₃ | Cl |
| 46 | 0 | 1 | benzo[b]thiophene-2,6-diyl | 2 | ethyne-1,2-diyl | 2 | 3-methyl-isooxazol-4,5-diyl | 5 | H | H |
| 47 | 0 | 0 | 1H-indol-2,6-diyl | 2 | ethyne-1,2-diyl | 2 | 3-methyl-isooxazol-4,5-diyl | 5 | CH₃ | CH₃ |
| 48 | 0 | 1 | 1H-indol-2,6-diyl | 2 | ethyne-1,2-diyl | 2 | 3-methyl-isooxazol-4,5-diyl | 5 | CH₃ | Cl |
| 49 | 1 | 0 | cyclohexane-1,4-diyl | 4 | ethyne-1,2-diyl | 2 | 3-methyl-isothiazol-4,5-diyl | 5 | CH₃ | H |
| 50 | 1 | 0 | cyclohexane-1,4-diyl | 4 | ethyne-1,2-diyl | 2 | 3-methyl-isothiazol-4,5-diyl | 5 | CH₃ | CH₃ |
| 51 | 1 | 0 | cyclohexane-1,4-diyl | 4 | ethyne-1,2-diyl | 2 | 3-methyl-isothiazol-4,5-diyl | 5 | CH₃ | Cl |
| 52 | 1 | 0 | cyclohexane-1,4-diyl | 4 | ethyne-1,2-diyl | 2 | 3-methyl-isothiazol-4,5-diyl | 5 | H | H |
| 53 | 1 | 0 | cyclohexane-1,4-diyl | 4 | ethyne-1,2-diyl | 2 | 3-methyl-isothiazol-4,5-diyl | 5 | H | CH₃ |
| 54 | 1 | 0 | cyclohexane-1,4-diyl | 4 | ethyne-1,2-diyl | 2 | 3-methyl-isothiazol-4,5-diyl | 5 | H | Cl |
| 55 | 1 | 0 | cyclohex-1-en-1,4-diyl | 4 | ethyne-1,2-diyl | 2 | 3-methyl-isothiazol-4,5-diyl | 5 | CH₃ | H |
| 56 | 1 | 0 | cyclohex-1-en-1,4-diyl | 4 | ethyne-1,2-diyl | 2 | 3-methyl-isothiazol-4,5-diyl | 5 | CH₃ | CH₃ |
| 57 | 1 | 0 | cyclohex-1-en-1,4-diyl | 4 | ethyne-1,2-diyl | 2 | 3-methyl-isothiazol-4,5-diyl | 5 | CH₃ | Cl |
| 58 | 1 | 0 | cyclohex-1-en-1,4-diyl | 4 | ethyne-1,2-diyl | 2 | 3-methyl-isothiazol-4,5-diyl | 5 | H | H |
| 59 | 1 | 0 | cyclohex-1-en-1,4-diyl | 4 | ethyne-1,2-diyl | 2 | 3-methyl-isothiazol-4,5-diyl | 5 | H | CH₃ |
| 60 | 1 | 0 | cyclohex-1-en-1,4-diyl | 4 | ethyne-1,2-diyl | 2 | 3-methyl-isothiazol-4,5-diyl | 5 | H | Cl |
| 61 | 1 | 0 | 2,3-dihydrothieno[2,3-d]isothiazol- | 2 | ethyne-1,2-diyl | 2 | 3-methyl-isooxazol-4,5-diyl | 5 | CH₃ | H |
| | | | 2,5-diyl 1,1-dioxide | | | | | | | |
| 62 | 1 | 0 | 2,3-dihydrothieno[2,3-d]isothiazol- | 2 | ethyne-1,2-diyl | 2 | 3-methyl-isooxazol-4,5-diyl | 5 | CH₃ | CH₃ |
| | | | 2,5-diyl 1,1-dioxide | | | | | | | |
| 63 | 1 | 0 | 2,3-dihydrothieno[2,3-d]isothiazol- | 2 | ethyne-1,2-diyl | 2 | 3-methyl-isooxazol-4,5-diyl | 5 | CH₃ | Cl |
| | | | 2,5-diyl 1,1-dioxide | | | | | | | |
| 64 | 1 | 0 | 2,3-dihydrothieno[2,3-d]isothiazol- | 2 | ethyne-1,2-diyl | 2 | 3-methyl-isooxazol-4,5-diyl | 5 | H | H |
| | | | 2,5-diyl 1,1-dioxide | | | | | | | |
| 65 | 1 | 0 | 2,3-dihydrothieno[2,3-d]isothiazol- | 2 | ethyne-1,2-diyl | 2 | 3-methyl-isooxazol-4,5-diyl | 5 | H | CH₃ |
| | | | 2,5-diyl 1,1-dioxide | | | | | | | |
| 66 | 1 | 0 | 2,3-dihydrothieno[2,3-d]isothiazol- | 2 | ethyne-1,2-diyl | 2 | 3-methyl-isooxazol-4,5-diyl | 5 | H | Cl |
| | | | 2,5-diyl 1,1-dioxide | | | | | | | |
| 67 | 1 | 0 | 2,3-dihydrothieno[2,3-d]isothiazol- | 2 | ethyne-1,2-diyl | 2 | isooxazol-4,5-diyl | 5 | CH₃ | H |
| | | | 2,5-diyl 1,1-dioxide | | | | | | | |
| 68 | 1 | 0 | 2,3-dihydrothieno[2,3-d]isothiazol- | 2 | ethyne-1,2-diyl | 2 | isooxazol-4,5-diyl | 5 | CH₃ | CH₃ |
| | | | 2,5-diyl 1,1-dioxide | | | | | | | |
| 69 | 1 | 0 | 2,3-dihydrothieno[2,3-d]isothiazol- | 2 | ethyne-1,2-diyl | 2 | isooxazol-4,5-diyl | 5 | CH₃ | Cl |
| | | | 2,5-diyl 1,1-dioxide | | | | | | | |
| 70 | 1 | 0 | 2,3-dihydrothieno[2,3-d]isothiazol- | 2 | ethyne-1,2-diyl | 2 | isooxazol-4,5-diyl | 5 | H | H |
| | | | 2,5-diyl 1,1-dioxide | | | | | | | |
| 71 | 1 | 0 | 2,3-dihydrothieno[2,3-d]isothiazol- | 2 | ethyne-1,2-diyl | 2 | isooxazol-4,5-diyl | 5 | H | CH₃ |
| | | | 2,5-diyl 1,1-dioxide | | | | | | | |
| 72 | 1 | 0 | 2,3-dihydrothieno[2,3-d]isothiazol- | 2 | ethyne-1,2-diyl | 2 | isooxazol-4,5-diyl | 5 | H | Cl |
| | | | 2,5-diyl 1,1-dioxide | | | | | | | |
| 73 | 1 | 0 | 2,3-dihydrothieno[2,3-d]isothiazol- | 2 | ethyne-1,2-diyl | 2 | 3-methyl-isothiazol-4,5-diyl | 5 | CH₃ | H |
| | | | 2,5-diyl 1,1-dioxide | | | | | | | |
| 74 | 1 | 0 | 2,3-dihydrothieno[2,3-d]isothiazol- | 2 | ethyne-1,2-diyl | 2 | 3-methyl-isothiazol-4,5-diyl | 5 | CH₃ | CH₃ |
| | | | 2,5-diyl 1,1-dioxide | | | | | | | |
| 75 | 1 | 0 | 2,3-dihydrothieno[2,3-d]isothiazol- | 2 | ethyne-1,2-diyl | 2 | 3-methyl-isothiazol-4,5-diyl | 5 | CH₃ | Cl |
| | | | 2,5-diyl 1,1-dioxide | | | | | | | |
| 76 | 1 | 0 | 2,3-dihydrothieno[2,3-d]isothiazol- | 2 | ethyne-1,2-diyl | 2 | 3-methyl-isothiazol-4,5-diyl | 5 | H | H |
| | | | 2,5-diyl 1,1-dioxide | | | | | | | |
| 77 | 1 | 0 | 2,3-dihydrothieno[2,3-d]isothiazol- | 2 | ethyne-1,2-diyl | 2 | 3-methyl-isothiazol-4,5-diyl | 5 | H | CH₃ |
| | | | 2,5-diyl 1,1-dioxide | | | | | | | |
| 78 | 1 | 0 | 2,3-dihydrothieno[2,3-d]isothiazol- | 2 | ethyne-1,2-diyl | 2 | 3-methyl-isothiazol-4,5-diyl | 5 | H | Cl |
| | | | 2,5-diyl 1,1-dioxide | | | | | | | |
| 79 | 1 | 0 | 2,3-dihydrothieno[2,3-d]isothiazol- | 2 | ethyne-1,2-diyl | 2 | isothiazol-4,5-diyl | 5 | CH₃ | H |
| | | | 2,5-diyl 1,1-dioxide | | | | | | | |
| 80 | 1 | 0 | 2,3-dihydrothieno[2,3-d]isothiazol- | 2 | ethyne-1,2-diyl | 2 | isothiazol-4,5-diyl | 5 | CH₃ | CH₃ |
| | | | 2,5-diyl 1,1-dioxide | | | | | | | |
| 81 | 1 | 0 | 2,3-dihydrothieno[2,3-d]isothiazol- | 2 | ethyne-1,2-diyl | 2 | isothiazol-4,5-diyl | 5 | CH₃ | Cl |
| | | | 2,5-diyl 1,1-dioxide | | | | | | | |
| 82 | 1 | 0 | 2,3-dihydrothieno[2,3-d]isothiazol- | 2 | ethyne-1,2-diyl | 2 | isothiazol-4,5-diyl | 5 | H | H |
| | | | 2,5-diyl 1,1-dioxide | | | | | | | |
| 83 | 1 | 0 | 2,3-dihydrothieno[2,3-d]isothiazol- | 2 | ethyne-1,2-diyl | 2 | isothiazol-4,5-diyl | 5 | H | CH₃ |
| | | | 2,5-diyl 1,1-dioxide | | | | | | | |
| 84 | 1 | 0 | 2,3-dihydrothieno[2,3-d]isothiazol- | 2 | ethyne-1,2-diyl | 2 | isothiazol-4,5-diyl | 5 | H | Cl |
| | | | 2,5-diyl 1,1-dioxide | | | | | | | |
| 85 | 1 | 0 | 2,3-dihydrothieno[2,3-d]isothiazol- | 2 | ethyne-1,2-diyl | 2 | 5-methyl-isooxazol-3,4-diyl | 3 | CH₃ | H |
| | | | 2,5-diyl 1,1-dioxide | | | | | | | |
| 86 | 1 | 0 | 2,3-dihydrothieno[2,3-d]isothiazol- | 2 | ethyne-1,2-diyl | 2 | 5-methyl-isooxazol-3,4-diyl | 3 | CH₃ | CH₃ |
| | | | 2,5-diyl 1,1-dioxide | | | | | | | |
| 87 | 1 | 0 | 2,3-dihydrothieno[2,3-d]isothiazol- | 2 | ethyne-1,2-diyl | 2 | 5-methyl-isooxazol-3,4-diyl | 3 | CH₃ | Cl |
| | | | 2,5-diyl 1,1-dioxide | | | | | | | |
| 88 | 1 | 0 | 2,3-dihydrothieno[2,3-d]isothiazol- | 2 | ethyne-1,2-diyl | 2 | 5-methyl-isooxazol-3,4-diyl | 3 | H | H |
| | | | 2,5-diyl 1,1-dioxide | | | | | | | |
| 89 | 1 | 0 | 2,3-dihydrothieno[2,3-d]isothiazol- | 2 | ethyne-1,2-diyl | 2 | 5-methyl-isooxazol-3,4-diyl | 3 | H | CH₃ |
| | | | 2,5-diyl 1,1-dioxide | | | | | | | |
| 90 | 1 | 0 | 2,3-dihydrothieno[2,3-d]isothiazol- | 2 | ethyne-1,2-diyl | 2 | 5-methyl-isooxazol-3,4-diyl | 3 | H | Cl |
| | | | 2,5-diyl 1,1-dioxide | | | | | | | |
| 91 | 1 | 0 | 2,3-dihydrothieno[2,3-d]isothiazol- | 2 | ethyne-1,2-diyl | 2 | isooxazol-3,4-diyl | 3 | CH₃ | H |
| | | | 2,5-diyl 1,1-dioxide | | | | | | | |
| 92 | 1 | 0 | 2,3-dihydrothieno[2,3-d]isothiazol- | 2 | ethyne-1,2-diyl | 2 | isooxazol-3,4-diyl | 3 | CH₃ | CH₃ |
| | | | 2,5-diyl 1,1-dioxide | | | | | | | |
| 93 | 1 | 0 | 2,3-dihydrothieno[2,3-d]isothiazol- | 2 | ethyne-1,2-diyl | 2 | isooxazol-3,4-diyl | 3 | CH₃ | Cl |
| | | | 2,5-diyl 1,1-dioxide | | | | | | | |
| 94 | 1 | 0 | 2,3-dihydrothieno[2,3-d]isothiazol- | 2 | ethyne-1,2-diyl | 2 | isooxazol-3,4-diyl | 3 | H | H |
| | | | 2,5-diyl 1,1-dioxide | | | | | | | |
| 95 | 1 | 0 | 2,3-dihydrothieno[2,3-d]isothiazol- | 2 | ethyne-1,2-diyl | 2 | isooxazol-3,4-diyl | 3 | H | CH₃ |
| | | | 2,5-diyl 1,1-dioxide | | | | | | | |
| 96 | 1 | 0 | 2,3-dihydrothieno[2,3-d]isothiazol- | 2 | ethyne-1,2-diyl | 2 | isooxazol-3,4-diyl | 3 | H | Cl |
| | | | 2,5-diyl 1,1-dioxide | | | | | | | |
| 97 | 1 | 0 | 2,3-dihydrothieno[2,3-d]isothiazol- | 2 | ethyne-1,2-diyl | 2 | 1,2,5-oxadiazol-3,4-diyl | 3 | CH₃ | H |
| | | | 2,5-diyl 1,1-dioxide | | | | | | | |
| 98 | 1 | 0 | 2,3-dihydrothieno[2,3-d]isothiazol- | 2 | ethyne-1,2-diyl | 2 | 1,2,5-oxadiazol-3,4-diyl | 3 | CH₃ | CH₃ |
| | | | 2,5-diyl 1,1-dioxide | | | | | | | |
| 99 | 1 | 0 | 2,3-dihydrothieno[2,3-d]isothiazol- | 2 | ethyne-1,2-diyl | 2 | 1,2,5-oxadiazol-3,4-diyl | 3 | CH₃ | Cl |
| | | | 2,5-diyl 1,1-dioxide | | | | | | | |
| 100 | 1 | 0 | 2,3-dihydrothieno[2,3-d]isothiazol- | 2 | ethyne-1,2-diyl | 2 | 1,2,5-oxadiazol-3,4-diyl | 3 | H | H |
| | | | 2,5-diyl 1,1-dioxide | | | | | | | |
| 101 | 1 | 0 | 2,3-dihydrothieno[2,3-d]isothiazol- | 2 | ethyne-1,2-diyl | 2 | 1,2,5-oxadiazol-3,4-diyl | 3 | H | CH₃ |
| | | | 2,5-diyl 1,1-dioxide | | | | | | | |
| 102 | 1 | 0 | 2,3-dihydrothieno[2,3-d]isothiazol- | 2 | ethyne-1,2-diyl | 2 | 1,2,5-oxadiazol-3,4-diyl | 3 | H | Cl |
| | | | 2,5-diyl 1,1-dioxide | | | | | | | |
| 103 | 1 | 0 | 2,3-dihydrothieno[2,3-d]isothiazol- | 2 | ethyne-1,2-diyl | 2 | 1-methyl-1,2,3-triazol-4,5-diyl | 4 | CH₃ | H |
| | | | 2,5-diyl 1,1-dioxide | | | | | | | |
| 104 | 1 | 0 | 2,3-dihydrothieno[2,3-d]isothiazol- | 2 | ethyne-1,2-diyl | 2 | 1-methyl-1,2,3-triazol-4,5-diyl | 4 | CH₃ | CH₃ |
| | | | 2,5-diyl 1,1-dioxide | | | | | | | |
| 105 | 1 | 0 | 2,3-dihydrothieno[2,3-d]isothiazol- | 2 | ethyne-1,2-diyl | 2 | 1-methyl-1,2,3-triazol-4,5-diyl | 4 | CH₃ | Cl |
| | | | 2,5-diyl 1,1-dioxide | | | | | | | |
| 106 | 1 | 0 | 2,3-dihydrothieno[2,3-d]isothiazol- | 2 | ethyne-1,2-diyl | 2 | 1-methyl-1,2,3-triazol-4,5-diyl | 4 | H | H |
| | | | 2,5-diyl 1,1-dioxide | | | | | | | |
| 107 | 1 | 0 | 2,3-dihydrothieno[2,3-d]isothiazol- | 2 | ethyne-1,2-diyl | 2 | 1-methyl-1,2,3-triazol-4,5-diyl | 4 | H | CH₃ |
| | | | 2,5-diyl 1,1-dioxide | | | | | | | |
| 108 | 1 | 0 | 2,3-dihydrothieno[2,3-d]isothiazol- | 2 | ethyne-1,2-diyl | 2 | 1-methyl-1,2,3-triazol-4,5-diyl | 4 | H | Cl |
| | | | 2,5-diyl 1,1-dioxide | | | | | | | |

Note:
Column + indicates ring A's point of connection to the adjacent terminal moiety.
Column # indicates ring B's point of connection to the adjacent ring A.
Column * indicates ring C's point of connection to the adjacent ring B.

In some embodiments, compounds of Formula (VI) are selected from the following compounds as listed in Table 6.

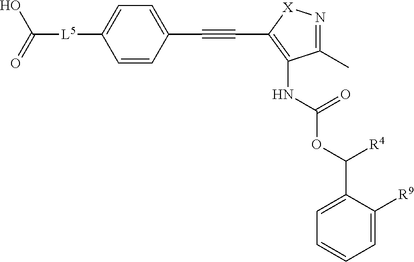

TABLE 6

| Cmpd # | | | | |
|---|---|---|---|---|
| VI- | L⁵ | X | R⁴ | R⁹ |
| 1A | (E)-ethylene-1,2-diyl | S | CH₃ | H |
| 2A | (E)-ethylene-1,2-diyl | S | CH₃ | Cl |
| 3A | (E)-ethylene-1,2-diyl | S | H | H |
| 4A | (E)-ethylene-1,2-diyl | S | CH₃ | CH₃ |
| 5A | ethyne-1,2-diyl | O | CH₃ | H |
| 6A | ethyne-1,2-diyl | O | CH₃ | Cl |

In some embodiments, compounds of Formula (VII) are also represented by Formula (VII-A) and are selected from the following compounds as listed in Table 7A.

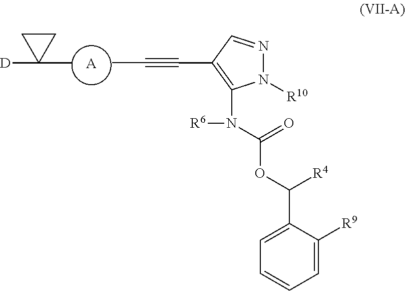

TABLE 7A

| Cmpd # VII- | D | 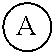 | # | R⁶ | R¹⁰ | R⁴ | R⁹ |
|---|---|---|---|---|---|---|---|
| 1A | carboxy | thieno[3,2-b]thiophene-2,5-diyl | 2 | H | CH₃ | CH₃ | H |
| 2A | carboxy | thieno[3,2-b]thiophene-2,5-diyl | 2 | H | CH₃ | CH₃ | Cl |
| 3A | carboxy | 3-methylthieno[3,2-b]thiophene-2,5-diyl | 2 | H | CH₃ | CH₃ | H |
| 4A | carboxy | 3-methylthieno[3,2-b]thiophene-2,5-diyl | 5 | H | CH₃ | CH₃ | CH₃ |
| 5A | carboxy | thieno[3,2-b]thiophene-2,5-diyl | 2 | H | CH₃ | H | H |
| 6A | carboxy | thieno[2,3-b]thiophene-2,5-diyl | 2 | H | CH₃ | CH₃ | H |
| 7A | carboxy | 3-methylthieno[2,3-b]thiophene-2,5-diyl | 2 | H | CH₃ | CH₃ | H |
| 8A | carboxy | 3-fluorothieno[3,2-b]thiophene-2,5-diyl | 2 | H | CH₃ | CH₃ | H |
| 9A | carboxy | 3-fluorothieno[3,2-b]thiophene-2,5-diyl | 5 | H | CH₃ | CH₃ | H |
| 10A | carboxy | 3-chlorothieno[3,2-b]thiophene-2,5-diyl | 2 | H | CH₃ | CH₃ | H |
| 11A | carboxy | 3-chlorothieno[3,2-b]thiophene-2,5-diyl | 5 | H | CH₃ | CH₃ | H |
| 12A | carboxy | 3-cyanothieno[3,2-b]thiophene-2,5-diyl | 2 | H | CH₃ | CH₃ | H |
| 13A | carboxy | 3-cyanothieno[3,2-b]thiophene-2,5-diyl | 5 | H | CH₃ | CH₃ | H |
| 14A | carboxy | 3-methoxythieno[3,2-b]thiophene-2,5-diyl | 2 | H | CH₃ | CH₃ | H |
| 15A | carboxy | 3-methoxythieno[3,2-b]thiophene-2,5-diyl | 5 | H | CH₃ | CH₃ | H |
| 16A | carboxy | 3-fluorothieno[2,3-b]thiophene-2,5-diyl | 2 | H | CH₃ | CH₃ | H |
| 17A | carboxy | 3-chlorothieno[2,3-b]thiophene-2,5-diyl | 2 | H | CH₃ | CH₃ | H |
| 18A | carboxy | 3-cyanothieno[2,3-b]thiophene-2,5-diyl | 2 | H | CH₃ | CH₃ | H |
| 19A | carboxy | 3-methoxythieno[2,3-b]thiophene-2,5-diyl | 2 | H | CH₃ | CH₃ | H |
| 20A | carboxy | thieno[3,2-b]thiophene-2,5-diyl | 2 | H | CH₃ | CH₃ | CH₃ |
| 21A | carboxy | 3-methylthieno[3,2-b]thiophene-2,5-diyl | 5 | H | CH₃ | CH₃ | H |
| 22A | carboxy | 3-methylthieno[3,2-b]thiophene-2,5-diyl | 5 | H | CH₃ | CH₃ | CH₃ |
| 23A | carboxy | 3-fluorothieno[3,2-b]thiophene-2,5-diyl | 2 | H | CH₃ | CH₃ | CH₃ |
| 24A | carboxy | 3-fluorothieno[3,2-b]thiophene-2,5-diyl | 2 | H | CH₃ | CH₃ | Cl |
| 25A | carboxy | 3-chlorothieno[3,2-b]thiophene-2,5-diyl | 5 | H | CH₃ | CH₃ | Cl |
| 26A | carboxy | 3-fluorothieno[2,3-b]thiophene-2,5-diyl | 2 | H | CH₃ | CH₃ | Cl |
| 27A | carboxy | 3-methoxythieno[2,3-b]thiophene-2,5-diyl | 2 | H | CH₃ | CH₃ | Cl |
| 28A | carboxy | thieno[2,3-b]thiophene-2,5-diyl | 2 | H | CH₃ | CH₃ | Cl |
| 29A | carboxy | 3-methylthieno[3,2-b]thiophene-2,5-diyl | 2 | H | CH₃ | CH₃ | Cl |
| 30A | carboxy | 3-methylthieno[3,2-b]thiophene-2,5-diyl | 5 | H | CH₃ | CH₃ | Cl |
| 31A | carboxy | thieno[3,2-b]thiophene-2,5-diyl | 2 | H | CH₃ | H | Cl |
| 32A | carboxy | thieno[2,3-b]thiophene-2,5-diyl | 2 | H | CH₃ | CH₃ | Cl |
| 33A | carboxy | 3-methylthieno[2,3-b]thiophene-2,5-diyl | 2 | H | CH₃ | CH₃ | Cl |
| 34A | carboxy | 3-fluorothieno[3,2-b]thiophene-2,5-diyl | 2 | H | CH₃ | CH₃ | Cl |
| 35A | carboxy | 3-fluorothieno[3,2-b]thiophene-2,5-diyl | 5 | H | CH₃ | CH₃ | Cl |
| 36A | carboxy | 3-chlorothieno[3,2-b]thiophene-2,5-diyl | 2 | H | CH₃ | CH₃ | Cl |
| 37A | carboxy | 3-chlorothieno[3,2-b]thiophene-2,5-diyl | 5 | H | CH₃ | CH₃ | Cl |
| 38A | carboxy | 3-cyanothieno[3,2-b]thiophene-2,5-diyl | 2 | H | CH₃ | CH₃ | Cl |
| 39A | carboxy | 3-cyanothieno[3,2-b]thiophene-2,5-diyl | 5 | H | CH₃ | CH₃ | Cl |
| 40A | carboxy | 3-methoxythieno[3,2-b]thiophene-2,5-diyl | 2 | H | CH₃ | CH₃ | Cl |
| 41A | carboxy | 3-methoxythieno[3,2-b]thiophene-2,5-diyl | 5 | H | CH₃ | CH₃ | Cl |
| 42A | carboxy | 3-fluorothieno[2,3-b]thiophene-2,5-diyl | 2 | H | CH₃ | CH₃ | Cl |
| 43A | carboxy | 3-chlorothieno[2,3-b]thiophene-2,5-diyl | 2 | H | CH₃ | CH₃ | Cl |
| 44A | carboxy | 3-cyanothieno[2,3-b]thiophene-2,5-diyl | 2 | H | CH₃ | CH₃ | Cl |
| 45A | carboxy | 3-methoxythieno[2,3-b]thiophene-2,5-diyl | 2 | H | CH₃ | CH₃ | Cl |
| 46A | carboxy | 3-fluorothieno[3,2-b]thiophene-2,5-diyl | 5 | H | CH₃ | CH₃ | Cl |
| 47A | carboxy | 3-cyanothieno[3,2-b]thiophene-2,5-diyl | 2 | H | CH₃ | CH₃ | Cl |
| 48A | carboxy | 3-methoxythieno[3,2-b]thiophene-2,5-diyl | 5 | H | CH₃ | CH₃ | Cl |
| 49A | carboxy | 3-methylthieno[3,2-b]thiophene-2,5-diyl | 2 | H | CH₃ | CH₃ | Cl |
| 50A | carboxy | 3-methylthieno[3,2-b]thiophene-2,5-diyl | 5 | H | CH₃ | CH₃ | Cl |
| 51A | carboxy | thieno[2,3-b]thiophene-2,5-diyl | 2 | H | CH₃ | CH₃ | Cl |
| 52A | carboxy | 3-methylthieno[2,3-b]thiophene-2,5-diyl | 2 | H | CH₃ | CH₃ | Cl |
| 53A | carboxy | 3-fluorothieno[3,2-b]thiophene-2,5-diyl | 2 | H | CH₃ | CH₃ | Cl |
| 54A | carboxy | 3-chlorothieno[3,2-b]thiophene-2,5-diyl | 2 | H | CH₃ | CH₃ | Cl |
| 55A | carboxy | 3-chlorothieno[3,2-b]thiophene-2,5-diyl | 5 | H | CH₃ | CH₃ | Cl |
| 56A | carboxy | 3-cyanothieno[3,2-b]thiophene-2,5-diyl | 5 | H | CH₃ | CH₃ | Cl |
| 57A | carboxy | 3-methoxythieno[3,2-b]thiophene-2,5-diyl | 2 | H | CH₃ | CH₃ | Cl |
| 58A | carboxy | 3-fluorothieno[2,3-b]thiophene-2,5-diyl | 2 | H | CH₃ | CH₃ | Cl |
| 59A | carboxy | 3-chlorothieno[2,3-b]thiophene-2,5-diyl | 2 | H | CH₃ | CH₃ | Cl |
| 60A | carboxy | 3-cyanothieno[2,3-b]thiophene-2,5-diyl | 2 | H | CH₃ | CH₃ | Cl |
| 61A | carboxy | 3-methoxythieno[2,3-b]thiophene-2,5-diyl | 2 | H | CH₃ | CH₃ | Cl |
| 62A | carboxy | 3-methylthieno[3,2-b]thiophene-2,5-diyl | 2 | H | CH₃ | CH₃ | CH₃ |
| 63A | carboxy | 3-methylthieno[3,2-b]thiophene-2,5-diyl | 2 | H | CH₃ | CH₃ | CH₃ |
| 64A | carboxy | 3-methylthieno[3,2-b]thiophene-2,5-diyl | 2 | H | CH₃ | H | H |
| 65A | carboxy | thieno[2,3-b]thiophene-2,5-diyl | 2 | H | CH₃ | CH₃ | CH₃ |
| 66A | carboxy | 3-fluorothieno[3,2-b]thiophene-2,5-diyl | 2 | H | CH₃ | CH₃ | CH₃ |
| 67A | carboxy | 3-chlorothieno[3,2-b]thiophene-2,5-diyl | 5 | H | CH₃ | CH₃ | CH₃ |
| 68A | carboxy | 3-fluorothieno[2,3-b]thiophene-2,5-diyl | 2 | H | CH₃ | CH₃ | CH₃ |
| 69A | carboxy | 3-methoxythieno[2,3-b]thiophene-2,5-diyl | 2 | H | CH₃ | CH₃ | CH₃ |
| 70A | carboxy | thieno[3,2-b]thiophene-2,5-diyl | 2 | H | CH₃ | H | CH₃ |
| 71A | carboxy | thieno[2,3-b]thiophene-2,5-diyl | 2 | H | CH₃ | CH₃ | CH₃ |
| 72A | carboxy | 3-fluorothieno[3,2-b]thiophene-2,5-diyl | 2 | H | CH₃ | CH₃ | CH₃ |
| 73A | carboxy | 3-chlorothieno[3,2-b]thiophene-2,5-diyl | 5 | H | CH₃ | CH₃ | CH₃ |
| 74A | carboxy | 3-fluorothieno[2,3-b]thiophene-2,5-diyl | 2 | H | CH₃ | CH₃ | CH₃ |
| 75A | carboxy | 3-chlorothieno[2,3-b]thiophene-2,5-diyl | 2 | H | CH₃ | CH₃ | CH₃ |
| 76A | carboxy | 3-methoxythieno[2,3-b]thiophene-2,5-diyl | 2 | H | CH₃ | CH₃ | CH₃ |
| 77A | carboxy | 3-methylthieno[3,2-b]thiophene-2,5-diyl | 5 | H | CH₃ | H | H |
| 78A | carboxy | thieno[2,3-b]thiophene-2,5-diyl | 2 | H | CH₃ | CH₃ | CH₃ |
| 79A | carboxy | 3-methylthieno[2,3-b]thiophene-2,5-diyl | 2 | H | CH₃ | CH₃ | CH₃ |
| 80A | carboxy | 3-fluorothieno[3,2-b]thiophene-2,5-diyl | 2 | H | CH₃ | CH₃ | CH₃ |
| 81A | carboxy | 3-chlorothieno[3,2-b]thiophene-2,5-diyl | 2 | H | CH₃ | CH₃ | CH₃ |
| 82A | carboxy | 3-chlorothieno[3,2-b]thiophene-2,5-diyl | 5 | H | CH₃ | CH₃ | CH₃ |
| 83A | carboxy | 3-cyanothieno[3,2-b]thiophene-2,5-diyl | 2 | H | CH₃ | CH₃ | CH₃ |
| 84A | carboxy | 3-methoxythieno[3,2-b]thiophene-2,5-diyl | 2 | H | CH₃ | CH₃ | CH₃ |
| 85A | carboxy | 3-methoxythieno[3,2-b]thiophene-2,5-diyl | 5 | H | CH₃ | CH₃ | CH₃ |
| 86A | carboxy | 3-fluorothieno[2,3-b]thiophene-2,5-diyl | 2 | H | CH₃ | CH₃ | CH₃ |
| 87A | carboxy | 3-chlorothieno[2,3-b]thiophene-2,5-diyl | 2 | H | CH₃ | CH₃ | CH₃ |
| 88A | carboxy | 3-cyanothieno[2,3-b]thiophene-2,5-diyl | 2 | H | CH₃ | CH₃ | CH₃ |
| 89A | carboxy | 3-methoxythieno[2,3-b]thiophene-2,5-diyl | 2 | H | CH₃ | CH₃ | CH₃ |
| 90A | carboxy | 3-fluorothieno[3,2-b]thiophene-2,5-diyl | 5 | H | CH₃ | CH₃ | CH₃ |
| 91A | carboxy | 3-methylthieno[3,2-b]thiophene-2,5-diyl | 5 | H | CH₃ | CH₃ | H |
| 92A | carboxy | 3-cyanothieno[3,2-b]thiophene-2,5-diyl | 5 | H | CH₃ | CH₃ | CH₃ |
| 93A | carboxy | 3-methoxythieno[3,2-b]thiophene-2,5-diyl | 5 | H | CH₃ | H | H |
| 94A | carboxy | thieno[2,3-b]thiophene-2,5-diyl | 2 | H | CH₃ | H | H |
| 95A | carboxy | 3-methylthieno[2,3-b]thiophene-2,5-diyl | 2 | H | CH₃ | H | H |
| 96A | carboxy | 3-fluorothieno[3,2-b]thiophene-2,5-diyl | 2 | H | CH₃ | H | H |
| 97A | carboxy | 3-fluorothieno[3,2-b]thiophene-2,5-diyl | 5 | H | CH₃ | H | H |
| 98A | carboxy | 3-chlorothieno[3,2-b]thiophene-2,5-diyl | 2 | H | CH₃ | H | H |
| 99A | carboxy | 3-chlorothieno[3,2-b]thiophene-2,5-diyl | 5 | H | CH₃ | H | H |
| 100A | carboxy | 3-cyanothieno[3,2-b]thiophene-2,5-diyl | 2 | H | CH₃ | H | H |
| 101A | carboxy | 3-cyanothieno[3,2-b]thiophene-2,5-diyl | 5 | H | CH₃ | H | H |
| 102A | carboxy | 3-methoxythieno[3,2-b]thiophene-2,5-diyl | 2 | H | CH₃ | H | H |
| 103A | carboxy | 3-fluorothieno[2,3-b]thiophene-2,5-diyl | 2 | H | CH₃ | H | H |
| 104A | carboxy | 3-chlorothieno[2,3-b]thiophene-2,5-diyl | 2 | H | CH₃ | H | H |
| 105A | carboxy | 3-cyanothieno[2,3-b]thiophene-2,5-diyl | 2 | H | CH₃ | H | H |
| 106A | carboxy | 3-methoxythieno[2,3-b]thiophene-2,5-diyl | 2 | H | CH₃ | H | H |
| 107A | carboxy | 3-methylthieno[3,2-b]thiophene-2,5-diyl | 5 | H | CH₃ | CH₃ | H |
| 108A | carboxy | 3,6-dicyanothieno[3,2-b]thiophene-2,5-diyl | 2 | H | CH₃ | CH₃ | Cl |
| 109A | carboxy | 3,6-dimethylthieno[3,2-b]thiophene-2,5-diyl | 2 | H | CH₃ | CH₃ | Cl |
| 110A | carboxy | 3,6-difluorothieno[3,2-b]thiophene-2,5-diyl | 2 | H | CH₃ | CH₃ | Cl |
| 111A | carboxy | 3,6-dichlorothieno[3,2-b]thiophene-2,5-diyl | 2 | H | CH₃ | CH₃ | Cl |
| 112A | carboxy | 3,4-dimethylthieno[2,3-b]thiophene-2,5-diyl | 2 | H | CH₃ | CH₃ | Cl |
| 113A | carboxy | 3,4-difluorothieno[2,3-b]thiophene-2,5-diyl | 2 | H | CH₃ | CH₃ | Cl |
| 114A | carboxy | 3,4-dichlorothieno[2,3-b]thiophene-2,5-diyl | 2 | H | CH₃ | CH₃ | Cl |
| 115A | carboxy | 3,4-dicyanothieno[2,3-b]thiophene-2,5-diyl | 2 | H | CH₃ | CH₃ | Cl |
| 116A | carboxy | 3,6-dicyanothieno[3,2-b]thiophene-2,5-diyl | 2 | H | CH₃ | CH₃ | Cl |
| 117A | carboxy | 3,6-dimethylthieno[3,2-b]thiophene-2,5-diyl | 2 | H | CH₃ | CH₃ | Cl |
| 118A | carboxy | 3,6-difluorothieno[3,2-b]thiophene-2,5-diyl | 2 | H | CH₃ | CH₃ | Cl |
| 119A | carboxy | 3,6-dichlorothieno[3,2-b]thiophene-2,5-diyl | 2 | H | CH₃ | CH₃ | Cl |
| 120A | carboxy | 3,4-dimethylthieno[2,3-b]thiophene-2,5-diyl | 2 | H | CH₃ | CH₃ | Cl |
| 121A | carboxy | 3,4-difluorothieno[2,3-b]thiophene-2,5-diyl | 2 | H | CH₃ | CH₃ | Cl |
| 122A | carboxy | 3,4-dichlorothieno[2,3-b]thiophene-2,5-diyl | 2 | H | CH₃ | CH₃ | Cl |
| 123A | carboxy | 3,4-dicyanothieno[2,3-b]thiophene-2,5-diyl | 2 | H | CH₃ | CH₃ | Cl |
| 124A | carboxy | 3,6-dicyanothieno[3,2-b]thiophene-2,5-diyl | 2 | H | CH₃ | CH₃ | Cl |
| 125A | carboxy | thieno[3,2-b]thiophene-2,5-diyl | 2 | H | H | CH₃ | H |
| 126A | carboxy | thieno[3,2-b]thiophene-2,5-diyl | 2 | H | H | CH₃ | CH₃ |
| 127A | carboxy | thieno[3,2-b]thiophene-2,5-diyl | 2 | H | H | CH₃ | Cl |
| 128A | carboxy | thieno[3,2-b]thiophene-2,5-diyl | 2 | CH₃ | CH₃ | CH₃ | H |
| 129A | carboxy | thieno[3,2-b]thiophene-2,5-diyl | 2 | CH₃ | CH₃ | CH₃ | CH₃ |
| 130A | carboxy | thieno[3,2-b]thiophene-2,5-diyl | 2 | CH₃ | CH₃ | CH₃ | Cl |
| 131A | carboxy | 3,6-dimethylthieno[3,2-b]thiophene-2,5-diyl | 2 | H | CH₃ | CH₃ | H |
| 132A | carboxy | 3,6-dimethylthieno[3,2-b]thiophene-2,5-diyl | 2 | H | CH₃ | CH₃ | CH₃ |
| 133A | tetrazol-5-yl | thieno[3,2-b]thiophene-2,5-diyl | 2 | H | CH₃ | CH₃ | H |
| 134A | tetrazol-5-yl | thieno[3,2-b]thiophene-2,5-diyl | 2 | H | CH₃ | CH₃ | CH₃ |
| 135A | tetrazol-5-yl | thieno[3,2-b]thiophene-2,5-diyl | 2 | H | CH₃ | CH₃ | Cl |
| 136A | (methylsulfonamidyl)- | thieno[3,2-b]thiophene-2,5-diyl | 2 | H | CH₃ | CH₃ | H |
| | N-carbonyl | | | | | | |
| 137A | (methylsulfonamidyl)- | thieno[3,2-b]thiophene-2,5-diyl | 2 | H | CH₃ | CH₃ | CH₃ |
| | N-carbonyl | | | | | | |
| 138A | (methylsulfonamidyl)- | thieno[3,2-b]thiophene-2,5-diyl | 2 | H | CH₃ | CH₃ | Cl |
| | N-carbonyl | | | | | | |
| 139A | 4-hydroxy-1,2,5- | thieno[3,2-b]thiophene-2,5-diyl | 2 | H | CH₃ | CH₃ | H |
| | oxadiazol-3-yl | | | | | | |
| 140A | 4-hydroxy-1,2,5- | thieno[3,2-b]thiophene-2,5-diyl | 2 | H | CH₃ | CH₃ | CH₃ |
| | oxadiazol-3-yl | | | | | | |
| 141A | 4-hydroxy-1,2,5- | thieno[3,2-b]thiophene-2,5-diyl | 2 | H | CH₃ | CH₃ | Cl |
| | oxadiazol-3-yl | | | | | | |
| 142A | 4-hydroxy-1,2,5- | thieno[3,2-b]thiophene-2,5-diyl | 2 | H | CH₃ | CH₃ | H |
| | thiadiazol-3-yl | | | | | | |
| 143A | 4-hydroxy-1,2,5- | thieno[3,2-b]thiophene-2,5-diyl | 2 | H | CH₃ | CH₃ | CH₃ |
| | thiadiazol-3-yl | | | | | | |
| 144A | 4-hydroxy-1,2,5- | thieno[3,2-b]thiophene-2,5-diyl | 2 | H | CH₃ | CH₃ | Cl |
| | thiadiazol-3-yl | | | | | | |
| 145A | 4-hydroxycyclobut-3- | thieno[3,2-b]thiophene-2,5-diyl | 2 | H | CH₃ | CH₃ | H |
| | ene-1,2-dione-3-aminyl | | | | | | |
| 146A | 4-hydroxycyclobut-3- | thieno[3,2-b]thiophene-2,5-diyl | 2 | H | CH₃ | CH₃ | CH₃ |
| | ene-1,2-dione-3-aminyl | | | | | | |
| 147A | 4-hydroxycyclobut-3- | thieno[3,2-b]thiophene-2,5-diyl | 2 | H | CH₃ | CH₃ | Cl |
| | ene-1,2-dione-3-aminyl | | | | | | |
| 148A | 4-hydroxycyclobut-3- | thieno[3,2-b]thiophene-2,5-diyl | 2 | H | CH₃ | H | H |
| | ene-1,2-dione-3-aminyl | | | | | | |
| 149A | 4-hydroxycyclobut-3- | thieno[3,2-b]thiophene-2,5-diyl | 2 | H | CH₃ | H | CH₃ |
| | ene-1,2-dione-3-aminyl | | | | | | |
| 150A | 4-hydroxycyclobut-3- | thieno[3,2-b]thiophene-2,5-diyl | 2 | H | CH₃ | H | Cl |
| | ene-1,2-dione-3-aminyl | | | | | | |
| 151A | carboxy | thieno[3,2-b]thiophene-2,5-diyl | 2 | H | H | H | H |
| 152A | carboxy | thieno[3,2-b]thiophene-2,5-diyl | 2 | H | H | H | CH₃ |
| 153A | carboxy | thieno[3,2-b]thiophene-2,5-diyl | 2 | H | H | H | Cl |
| 154A | carboxy | thieno[3,2-b]thiophene-2,5-diyl | 2 | CH₃ | CH₃ | H | H |
| 155A | carboxy | thieno[3,2-b]thiophene-2,5-diyl | 2 | CH₃ | CH₃ | H | CH₃ |
| 156A | carboxy | thieno[3,2-b]thiophene-2,5-diyl | 2 | CH₃ | CH₃ | H | Cl |
| 157A | carboxy | 3-methylthieno[3,2-b]thiophene-2,5-diyl | 2 | H | CH₃ | H | H |
| 158A | carboxy | 3-methylthieno[3,2-b]thiophene-2,5-diyl | 2 | H | CH₃ | H | CH₃ |
| 159A | carboxy | 3-methylthieno[3,2-b]thiophene-2,5-diyl | 2 | H | CH₃ | H | Cl |
| 160A | carboxy | 3,6-dimethylthieno[3,2-b]thiophene-2,5-diyl | 2 | H | CH₃ | H | H |
| 161A | carboxy | 3,6-dimethylthieno[3,2-b]thiophene-2,5-diyl | 2 | H | CH₃ | H | CH₃ |
| 162A | carboxy | 3,6-dimethylthieno[3,2-b]thiophene-2,5-diyl | 2 | H | CH₃ | H | Cl |
| 163A | tetrazol-5-yl | thieno[3,2-b]thiophene-2,5-diyl | 2 | H | CH₃ | H | H |
| 164A | tetrazol-5-yl | thieno[3,2-b]thiophene-2,5-diyl | 2 | H | CH₃ | H | CH₃ |
| 165A | tetrazol-5-yl | thieno[3,2-b]thiophene-2,5-diyl | 2 | H | CH₃ | H | Cl |
| 166A | (methylsulfonamidyl)- | thieno[3,2-b]thiophene-2,5-diyl | 2 | H | CH₃ | H | H |
| | N-carbonyl | | | | | | |
| 167A | (methylsulfonamidyl)- | thieno[3,2-b]thiophene-2,5-diyl | 2 | H | CH₃ | H | CH₃ |
| | N-carbonyl | | | | | | |
| 168A | (methylsulfonamidyl)- | thieno[3,2-b]thiophene-2,5-diyl | 2 | H | CH₃ | H | Cl |
| | N-carbonyl | | | | | | |
| 169A | 4-hydroxy-1,2,5- | thieno[3,2-b]thiophene-2,5-diyl | 2 | H | CH₃ | H | H |
| | oxadiazol-3-yl | | | | | | |
| 170A | 4-hydroxy-1,2,5- | thieno[3,2-b]thiophene-2,5-diyl | 2 | H | CH₃ | H | CH₃ |
| | oxadiazol-3-yl | | | | | | |
| 171A | 4-hydroxy-1,2,5- | thieno[3,2-b]thiophene-2,5-diyl | 2 | H | CH₃ | H | Cl |
| | oxadiazol-3-yl | | | | | | |
| 172A | 4-hydroxy-1,2,5- | thieno[3,2-b]thiophene-2,5-diyl | 2 | H | CH₃ | H | H |
| | thiadiazol-3-yl | | | | | | |
| 173A | 4-hydroxy-1,2,5- | thieno[3,2-b]thiophene-2,5-diyl | 2 | H | CH₃ | H | CH₃ |
| | thiadiazol-3-yl | | | | | | |
| 174A | 4-hydroxy-1,2,5- | thieno[3,2-b]thiophene-2,5-diyl | 2 | H | CH₃ | H | Cl |
| | thiadiazol-3-yl | | | | | | |
| 175A | carboxy | 2,3-dihydrothieno[2,3-d]isothiazol-2,5-diyl | 2 | H | CH₃ | CH₃ | H |
| | | 1,1-dioxide | | | | | |
| 176A | carboxy | 2,3-dihydrothieno[2,3-d]isothiazol-2,5-diyl | 2 | H | CH₃ | CH₃ | CH₃ |
| | | 1,1-dioxide | | | | | |
| 177A | carboxy | 2,3-dihydrothieno[2,3-d]isothiazol-2,5-diyl | 2 | H | CH₃ | CH₃ | Cl |
| | | 1,1-dioxide | | | | | |
| 178A | carboxy | 2,3-dihydrothieno[2,3-d]isothiazol-2,5-diyl | 2 | H | CH₃ | H | H |
| | | 1,1-dioxide | | | | | |
| 179A | carboxy | 2,3-dihydrothieno[2,3-d]isothiazol-2,5-diyl | 2 | H | CH₃ | H | CH₃ |
| | | 1,1-dioxide | | | | | |
| 180A | carboxy | 2,3-dihydrothieno[2,3-d]isothiazol-2,5-diyl | 2 | H | CH₃ | H | Cl |
| | | 1,1-dioxide | | | | | |
| 181A | carboxy | 2,3-dihydrothieno[2,3-d]isothiazol-2,5-diyl | 2 | H | H | CH₃ | H |
| | | 1,1-dioxide | | | | | |
| 182A | carboxy | 2,3-dihydrothieno[2,3-d]isothiazol-2,5-diyl | 2 | H | H | CH₃ | CH₃ |
| | | 1,1-dioxide | | | | | |
| 183A | carboxy | 2,3-dihydrothieno[2,3-d]isothiazol-2,5-diyl | 2 | H | H | CH₃ | Cl |
| | | 1,1-dioxide | | | | | |
| 184A | carboxy | 2,3-dihydrothieno[2,3-d]isothiazol-2,5-diyl | 2 | H | H | H | H |
| | | 1,1-dioxide | | | | | |
| 185A | carboxy | 2,3-dihydrothieno[2,3-d]isothiazol-2,5-diyl | 2 | H | H | H | CH₃ |
| | | 1,1-dioxide | | | | | |
| 186A | carboxy | 2,3-dihydrothieno[2,3-d]isothiazol-2,5-diyl | 2 | H | H | H | Cl |
| | | 1,1-dioxide | | | | | |

Note:
Column # indicates ring A's point of connection to the adjacent terminal moiety.

In some embodiments, compounds of Formula (VII) are also represented by Formula (VII-B) and are selected from the following compounds as listed in Table 7B.

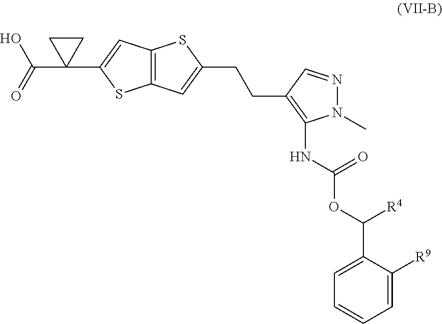

TABLE 7B

| Cmpd # | | |
|---|---|---|
| VII- | R⁴ | R⁹ |
| 1B | CH₃ | H |
| 2B | CH₃ | CH₃ |
| 3B | CH₃ | Cl |
| 4B | H | H |
| 5B | H | CH₃ |
| 6B | H | Cl |

In some embodiments, compounds of Formula (VII) are also represented by Formula (VII-C) and are selected from the following compounds as listed in Table 7C.

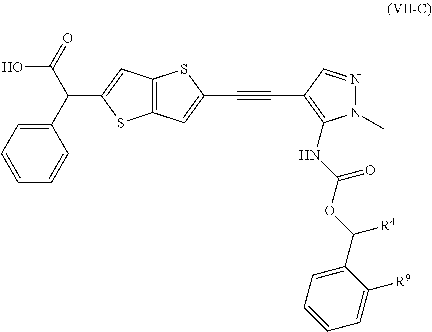

TABLE 7C

| Cmpd # | | |
|---|---|---|
| VII- | R⁴ | R⁹ |
| 1C | CH₃ | H |
| 2C | CH₃ | CH₃ |
| 3C | CH₃ | Cl |
| 4C | H | H |
| 5C | H | CH₃ |
| 6C | H | Cl |

In some embodiments, compounds of Formula (VII) are also represented by Formula (VII-D) and are selected from the following compounds as listed in Table 7D.

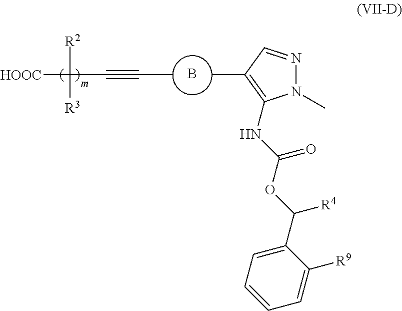

TABLE 7D

| Cmpd # VII- | R²/R³ | m | 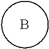 | # | R⁴ | R⁹ |
|---|---|---|---|---|---|---|
| 1D | n/a | 0 | naphthalen-2,6-diyl | 2 | CH₃ | H |
| 2D | 1,1-cyclopropyl | 1 | 1,4-phenylene | 4 | CH₃ | CH₃ |
| 3D | H/H | 1 | naphthalen-2,6-diyl | 2 | CH₃ | Cl |
| 4D | n/a | 0 | naphthalen-2,6-diyl | 2 | CH₃ | H |
| 5D | 1,1-cyclopropyl | 1 | quinolin-2,6-diyl | 2 | CH₃ | CH₃ |
| 6D | 1,1-cyclopropyl | 1 | quinolin-2,6-diyl | 6 | CH₃ | Cl |

Note:
Column # indicates ring B's point of connection to the adjacent ethyne-1,2-diyl moiety.

In some embodiments, compounds of Formula (VIII) are selected from the following compounds as listed in Table 8.

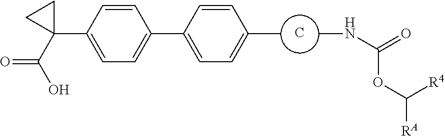

TABLE 8

| Cmpd # VIII- | 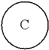 | * | R⁴ | R^{A} |
|---|---|---|---|---|
| 1 | 4H-furo[3,2-b]pyrrole-2,3-diyl | 2 | methyl | 2-chloro-phenyl |
| 2 | furo[3,2-b]furan-2,3-diyl | 2 | H | phenyl |
| 3 | 6H-furo[2,3-b]pyrrole-2,3-diyl | 2 | methyl | phenyl |
| 4 | furo[2,3-b]furan-2,3-diyl | 2 | methyl | 2-chloro-phenyl |
| 5 | thieno[2,3-b]furan-2,3-diyl | 2 | H | phenyl |
| 6 | 3H-pyrazol-3-one-4,5-diyl | 5 | methyl | phenyl |
| 7 | 3H-pyrazol-3-one-4,5-diyl | 5 | methyl | 2-methyl-phenyl |
| 8 | 3H-pyrazol-3-one-4,5-diyl | 5 | methyl | 2-chloro-phenyl |
| 9 | 3H-pyrazol-3-one-4,5-diyl | 5 | H | phenyl |
| 10 | 2H-imidazol-2-one-4,5-diyl | 4 | methyl | phenyl |
| 11 | 2H-imidazol-2-one-4,5-diyl | 4 | methyl | 2-methyl-phenyl |
| 12 | 2H-imidazol-2-one-4,5-diyl | 4 | methyl | 2-chloro-phenyl |
| 13 | 2-methylisothiazol-5(2H)-one-3,4-diyl | 4 | methyl | phenyl |
| 14 | 2-methylisothiazol-5(2H)-one-3,4-diyl | 4 | methyl | 2-methyl-phenyl |
| 15 | 2-methylisothiazol-5(2H)-one-3,4-diyl | 4 | methyl | 2-chloro-phenyl |
| 16 | 3H-pyrazol-3-one-4,5-diyl | 4 | methyl | phenyl |
| 17 | 3H-pyrazol-3-one-4,5-diyl | 4 | methyl | 2-methyl-phenyl |
| 18 | 3H-pyrazol-3-one-4,5-diyl | 4 | methyl | 2-chloro-phenyl |
| 19 | 1-methyl-1,2-dihydro-3H-pyrazol-3- | 4 | methyl | phenyl |
| | one-4,5-diyl | | | |
| 20 | 1-methyl-1,2-dihydro-3H-pyrazol-3- | 4 | methyl | 2-methyl-phenyl |
| | one-4,5-diyl | | | |
| 21 | 1-methyl-1,2-dihydro-3H-pyrazol-3- | 4 | methyl | 2-chloro-phenyl |
| | one-4,5-diyl | | | |
| 22 | 3-methylthiazol-2(3H)-one-4,5-diyl | 5 | methyl | phenyl |
| 23 | 3-methylthiazol-2(3H)-one-4,5-diyl | 5 | methyl | 2-methyl-phenyl |
| 24 | 3-methylthiazol-2(3H)-one-4,5-diyl | 5 | methyl | 2-chloro-phenyl |
| 25 | 2-methoxy-1-methyl-1H-imidazole- | 4 | methyl | phenyl |
| | 4,5-diyl | | | |
| 26 | 2-methoxy-1-methyl-1H-imidazole- | 4 | methyl | 2-methyl-phenyl |
| | 4,5-diyl | | | |
| 27 | 2-methoxy-1-methyl-1H-imidazole- | 4 | methyl | 2-chloro-phenyl |
| | 4,5-diyl | | | |
| 28 | 2-methylisothiazol-5(2H)-one 1,1- | 4 | methyl | phenyl |
| | dioxide-3,4-diyl | | | |
| 29 | 2-methylisothiazol-5(2H)-one 1,1- | 4 | methyl | 2-methyl-phenyl |
| | dioxide-3,4-diyl | | | |
| 30 | 2-methylisothiazol-5(2H)-one 1,1- | 4 | methyl | 2-chloro-phenyl |
| | dioxide-3,4-diyl | | | |
| 31 | 2H-imidazol-2-one-4,5-diyl | 4 | H | phenyl |
| 32 | 2H-imidazol-2-one-4,5-diyl | 4 | H | 2-methyl-phenyl |
| 33 | 2H-imidazol-2-one-4,5-diyl | 4 | H | 2-chloro-phenyl |
| 34 | 2-methylisothiazol-5(2H)-one-3,4-diyl | 4 | H | phenyl |
| 35 | 2-methylisothiazol-5(2H)-one-3,4-diyl | 4 | H | 2-methyl-phenyl |
| 36 | 2-methylisothiazol-5(2H)-one-3,4-diyl | 4 | H | 2-chloro-phenyl |
| 37 | 3H-pyrazol-3-one-4,5-diyl | 4 | H | phenyl |
| 38 | 3H-pyrazol-3-one-4,5-diyl | 4 | H | 2-methyl-phenyl |
| 39 | 3H-pyrazol-3-one-4,5-diyl | 4 | H | 2-chloro-phenyl |
| 40 | 1-methyl-1,2-dihydro-3H-pyrazol-3- | 4 | H | phenyl |
| | one-4,5-diyl | | | |
| 41 | 1-methyl-1,2-dihydro-3H-pyrazol-3- | 4 | H | 2-methyl-phenyl |
| | one-4,5-diyl | | | |
| 42 | 1-methyl-1,2-dihydro-3H-pyrazol-3- | 4 | H | 2-chloro-phenyl |
| | one-4,5-diyl | | | |
| 43 | 3-methylthiazol-2(3H)-one-4,5-diyl | 5 | H | phenyl |
| 44 | 3-methylthiazol-2(3H)-one-4,5-diyl | 5 | H | 2-methyl-phenyl |
| 45 | 3-methylthiazol-2(3H)-one-4,5-diyl | 5 | H | 2-chloro-phenyl |
| 46 | 2-methoxy-1-methyl-1H-imidazole- | 4 | H | phenyl |
| | 4,5-diyl | | | |
| 47 | 2-methoxy-1-methyl-1H-imidazole- | 4 | H | 2-methyl-phenyl |
| | 4,5-diyl | | | |
| 48 | 2-methoxy-1-methyl-1H-imidazole- | 4 | H | 2-chloro-phenyl |
| | 4,5-diyl | | | |
| 49 | 2-methylisothiazol-5(2H)-one 1,1- | 4 | H | phenyl |
| | dioxide-3,4-diyl | | | |
| 50 | 2-methylisothiazol-5(2H)-one 1,1- | 4 | H | 2-methyl-phenyl |
| | dioxide-3,4-diyl | | | |
| 51 | 2-methylisothiazol-5(2H)-one 1,1- | 4 | H | 2-chloro-phenyl |
| | dioxide-3,4-diyl | | | |

Note:
Column * indicates ring C's point of connection to the adjacent phen-1,4-ylene.

In some embodiments, compounds of Formula (IX) are selected from the following compounds as listed in Table 9.

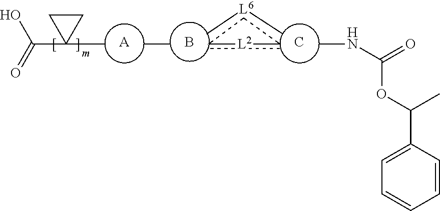

TABLE 9

| Cmpd # IX - | m | 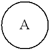 | 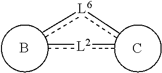 | * |
|---|---|---|---|---|
| 1 | 1 | 1,4-phenylene | 2-methyl-2,8-dihydroindeno | 6 |
| | | | [1,2-c]pyrrole-3,6-diyl | |
| 2 | 1 | 1,4-phenylene | 2-methyl-4,5-dihydro-2H-benzo | 7 |
| | | | [e]isoindole-1,7-diyl | |
| 3 | 0 | 1,4-phenylene | 2-methyl-2H-benzo | 7 |
| | | | [e]isoindole-1,7-diyl | |
| 4 | 0 | ethyne-1,2-diyl | 2-methyl-4,5-dihydro-2H-benzo | 7 |
| | | | [e]isoindole-1,7-diyl | |
| 5 | 0 | ethyne-1,2-diyl | 2-methyl-2H-benzo | 7 |
| | | | [e]isoindole-1,7-diyl | |
| 6 | 1 | ethyne-1,2-diyl | 2-methyl-2,8-dihydroindeno | 6 |
| | | | [1,2-c]pyrrole-3,6-diyl | |

Note: 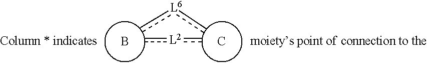
ring A.

In some embodiments, compounds of Formula (XI) are selected from the following compounds as listed in Table 10A. In some other embodiments, compounds described herein are selected from the following compounds as listed in Table 10B.

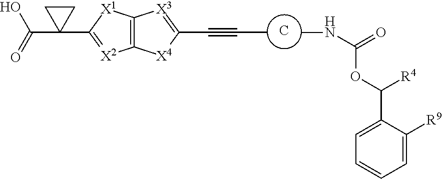

| | | | | | | | | |
|---|---|---|---|---|---|---|---|---|
| Cmpd # X- | X¹ | X² | X³ | X⁴ | 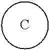 | * | R⁴ | R⁹ |

Table 10A.

| | | | | | | | | |
|---|---|---|---|---|---|---|---|---|
| 1A | S | CH | CH | S | 4-methyl 1,2,3-triazol-1,5-diyl | 1 | CH₃ | H |
| 2A | S | CH | CH | S | 4-methyl 1,2,3-triazol-1,5-diyl | 1 | CH₃ | CH₃ |
| 3A | S | CH | CH | S | 4-methyl 1,2,3-triazol-1,5-diyl | 1 | CH₃ | Cl |
| 4A | S | CH | CH | S | thiazole-4,5-diyl | 5 | CH₃ | H |
| 5A | S | CH | CH | S | thiazole-4,5-diyl | 5 | CH₃ | CH₃ |
| 6A | S | CH | CH | S | thiazole-4,5-diyl | 5 | CH₃ | Cl |
| 7A | S | CH | CH | S | oxazole-4,5-diyl | 5 | CH₃ | H |
| 8A | S | CH | CH | S | oxazole-4,5-diyl | 5 | CH₃ | CH₃ |
| 9A | S | CH | CH | S | oxazole-4,5-diyl | 5 | CH₃ | Cl |
| 10A | S | CH | CH | S | thiazole-4,5-diyl | 4 | CH₃ | H |
| 11A | S | CH | CH | S | thiazole-4,5-diyl | 4 | CH₃ | CH₃ |
| 12A | S | CH | CH | S | thiazole-4,5-diyl | 4 | CH₃ | Cl |
| 13A | S | CH | CH | S | isooxazol-4,5-diyl | 4 | CH₃ | H |
| 14A | S | CH | CH | S | isooxazol-4,5-diyl | 4 | CH₃ | CH₃ |
| 15A | S | CH | CH | S | isooxazol-4,5-diyl | 4 | CH₃ | Cl |
| 16A | S | CH | CH | S | isothiazol-4,5-diyl | 4 | CH₃ | H |
| 17A | S | CH | CH | S | isothiazol-4,5-diyl | 4 | CH₃ | CH₃ |
| 18A | S | CH | CH | S | isothiazol-4,5-diyl | 4 | CH₃ | Cl |
| 19A | S | CH | CH | S | 1,2,5-oxadiazol-3,4-diyl | 3 | CH₃ | H |
| 20A | S | CH | CH | S | 1,2,5-oxadiazol-3,4-diyl | 3 | CH₃ | CH₃ |
| 21A | S | CH | CH | S | 1,2,5-oxadiazol-3,4-diyl | 3 | CH₃ | Cl |
| 22A | S | CH | CH | S | pyridine-2,3-diyl | 2 | CH₃ | H |
| 23A | S | CH | CH | S | pyridine-2,3-diyl | 2 | CH₃ | CH₃ |
| 24A | S | CH | CH | S | pyridine-2,3-diyl | 2 | CH₃ | Cl |
| 25A | S | CH | CH | S | pyridine-3,4-diyl | 3 | CH₃ | H |
| 26A | S | CH | CH | S | pyridine-3,4-diyl | 3 | CH₃ | CH₃ |
| 27A | S | CH | CH | S | pyridine-3,4-diyl | 3 | CH₃ | Cl |
| 28A | S | CH | CH | S | pyridazine-3,4-diyl | 3 | CH₃ | H |
| 29A | S | CH | CH | S | pyridazine-3,4-diyl | 3 | CH₃ | CH₃ |
| 30A | S | CH | CH | S | pyridazine-3,4-diyl | 3 | CH₃ | Cl |
| 31A | S | CH | CH | S | imidazole-1,2-diyl | 1 | CH₃ | H |
| 32A | S | CH | CH | S | imidazole-1,2-diyl | 1 | CH₃ | CH₃ |
| 33A | S | CH | CH | S | imidazole-1,2-diyl | 1 | CH₃ | Cl |
| 34A | S | CH | CH | S | 4-methyl 1,2,3-triazol-1,5-diyl | 1 | H | H |
| 35A | S | CH | CH | S | 4-methyl 1,2,3-triazol-1,5-diyl | 1 | H | CH₃ |
| 36A | S | CH | CH | S | 4-methyl 1,2,3-triazol-1,5-diyl | 1 | H | Cl |
| 37A | S | CH | CH | S | imidazole-1,2-diyl | 1 | H | H |
| 38A | S | CH | CH | S | imidazole-1,2-diyl | 1 | H | CH₃ |
| 39A | S | CH | CH | S | imidazole-1,2-diyl | 1 | H | Cl |
| 40A | S | CH | CH | S | thiazole-4,5-diyl | 5 | H | H |
| 41A | S | CH | CH | S | thiazole-4,5-diyl | 5 | H | CH₃ |
| 42A | S | CH | CH | S | thiazole-4,5-diyl | 5 | H | Cl |
| 43A | S | CH | CH | S | oxazole-4,5-diyl | 5 | H | H |
| 44A | S | CH | CH | S | oxazole-4,5-diyl | 5 | H | CH₃ |
| 45A | S | CH | CH | S | oxazole-4,5-diyl | 5 | H | Cl |
| 46A | S | CH | CH | S | thiazole-4,5-diyl | 4 | H | H |
| 47A | S | CH | CH | S | thiazole-4,5-diyl | 4 | H | CH₃ |
| 48A | S | CH | CH | S | thiazole-4,5-diyl | 4 | H | Cl |
| 49A | S | CH | CH | S | isooxazol-4,5-diyl | 4 | H | H |
| 50A | S | CH | CH | S | isooxazol-4,5-diyl | 4 | H | CH₃ |
| 51A | S | CH | CH | S | isooxazol-4,5-diyl | 4 | H | Cl |
| 52A | S | CH | CH | S | isothiazol-4,5-diyl | 4 | H | H |
| 53A | S | CH | CH | S | isothiazol-4,5-diyl | 4 | H | CH₃ |
| 54A | S | CH | CH | S | isothiazol-4,5-diyl | 4 | H | Cl |
| 55A | S | CH | CH | S | 1,2,5-oxadiazol-3,4-diyl | 3 | H | H |
| 56A | S | CH | CH | S | 1,2,5-oxadiazol-3,4-diyl | 3 | H | CH₃ |
| 57A | S | CH | CH | S | 1,2,5-oxadiazol-3,4-diyl | 3 | H | Cl |
| 58A | S | CH | CH | S | pyridine-2,3-diyl | 2 | H | H |
| 59A | S | CH | CH | S | pyridine-2,3-diyl | 2 | H | CH₃ |
| 60A | S | CH | CH | S | pyridine-2,3-diyl | 2 | H | Cl |
| 61A | S | CH | CH | S | pyridine-3,4-diyl | 3 | H | H |
| 62A | S | CH | CH | S | pyridine-3,4-diyl | 3 | H | CH₃ |
| 63A | S | CH | CH | S | pyridine-3,4-diyl | 3 | H | Cl |
| 64A | S | CH | CH | S | pyridazine-3,4-diyl | 3 | H | H |
| 65A | S | CH | CH | S | pyridazine-3,4-diyl | 3 | H | CH₃ |
| 66A | S | CH | CH | S | pyridazine-3,4-diyl | 3 | H | Cl |

Table 10B.

| | | | | | | | | |
|---|---|---|---|---|---|---|---|---|
| 1B | S | CH | N | NH | 3-methyl isooxazol-4,5-diyl | 5 | CH₃ | H |
| 2B | S | CH | N | NH | 3-methyl isooxazol-4,5-diyl | 5 | CH₃ | CH₃ |
| 3B | S | CH | N | NH | 3-methyl isooxazol-4,5-diyl | 5 | CH₃ | Cl |
| 4B | S | CH | N | S | 3-methyl isooxazol-4,5-diyl | 5 | CH₃ | H |
| 5B | S | CH | N | S | 3-methyl isooxazol-4,5-diyl | 5 | CH₃ | CH₃ |
| 6B | S | CH | N | S | 3-methyl isooxazol-4,5-diyl | 5 | CH₃ | Cl |
| 7B | S | N | CH | S | 3-methyl isooxazol-4,5-diyl | 5 | CH₃ | H |
| 8B | S | N | CH | S | 3-methyl isooxazol-4,5-diyl | 5 | CH₃ | CH₃ |
| 9B | S | N | CH | S | 3-methyl isooxazol-4,5-diyl | 5 | CH₃ | Cl |
| 10B | NH | N | CH | S | 3-methyl isooxazol-4,5-diyl | 5 | CH₃ | H |
| 11B | NH | N | CH | S | 3-methyl isooxazol-4,5-diyl | 5 | CH₃ | CH₃ |
| 12B | NH | N | CH | S | 3-methyl isooxazol-4,5-diyl | 5 | CH₃ | Cl |
| 13B | S | CH | N | NH | 3-methyl isooxazol-4,5-diyl | 5 | H | H |
| 14B | S | CH | N | NH | 3-methyl isooxazol-4,5-diyl | 5 | H | CH₃ |
| 15B | S | CH | N | NH | 3-methyl isooxazol-4,5-diyl | 5 | H | Cl |
| 16B | S | CH | N | S | 3-methyl isooxazol-4,5-diyl | 5 | H | H |
| 17B | S | CH | N | S | 3-methyl isooxazol-4,5-diyl | 5 | H | CH₃ |
| 18B | S | CH | N | S | 3-methyl isooxazol-4,5-diyl | 5 | H | Cl |
| 19B | S | N | CH | S | 3-methyl isooxazol-4,5-diyl | 5 | H | H |
| 20B | S | N | CH | S | 3-methyl isooxazol-4,5-diyl | 5 | H | CH₃ |
| 21B | S | N | CH | S | 3-methyl isooxazol-4,5-diyl | 5 | H | Cl |
| 22B | NH | N | CH | S | 3-methyl isooxazol-4,5-diyl | 5 | H | H |
| 23B | NH | N | CH | S | 3-methyl isooxazol-4,5-diyl | 5 | H | CH₃ |
| 24B | NH | N | CH | S | 3-methyl isooxazol-4,5-diyl | 5 | H | Cl |

Note:
Column * indicates ring C's point of connection to the adjacent ethyne-l,2-diyl moiety.

Some embodiments of compounds described herein are selected from the following compounds as listed in Table 11A.

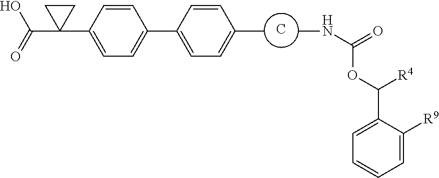

TABLE 11A

| Cmpd # XI- | 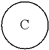 | * | R⁴ | R⁹ |
|---|---|---|---|---|
| 1A | 1-(trifluoromethyl)-1H-pyrazol-4,5-diyl | 4 | CH₃ | H |
| 2A | 1-(trifluoromethy0-1H-pyrazol-4,5-diyl | 4 | CH₃ | CH₃ |
| 3A | 1-(trifluoromethyl)-1H-pyrazol-4,5-diyl | 4 | CH₃ | Cl |
| 4A | 4-(trifluoromethyl)-1,2,3-triazol-1,5-diyl | 1 | CH₃ | H |
| 5A | 4-(trifluoromethy0-1,2,3-triazol-1,5-diyl | 1 | CH₃ | CH₃ |
| 6A | 4-(trifluoromethyl)-1,2,3-triazol-1,5-diyl | 1 | CH₃ | Cl |
| 7A | 1-(trifluoromethyl)-1H-pyrazol-4,5-diyl | 4 | H | H |
| 8A | 1-(trifluoromethyl)-1H-pyrazol-4,5-diyl | 4 | H | CH₃ |
| 9A | 1-(trifluoromethyl)-1H-pyrazol-4,5-diyl | 4 | H | Cl |
| 10A | 4-(trifluoromethy0-1,2,3-triazol-1,5-diyl | 1 | H | H |
| 11A | 4-(trifluoromethy0-1,2,3-triazol-1,5-diyl | 1 | H | CH₃ |
| 12A | 4-(trifluoromethy0-1,2,3-triazol-1,5-diyl | 1 | H | Cl |
| 13A | 2-methoxy-1-methyl-1H-imidazole-4,5-diyl | 4 | CH₃ | H |
| 14A | 2-methoxy-1-methyl-1H-imidazole-4,5-diyl | 4 | CH₃ | CH₃ |
| 15A | 2-methoxy-1-methyl-1H-imidazole-4,5-diyl | 4 | CH₃ | Cl |
| 16A | 3-methoxy-1-methyl-1H-pyrazol-4,5-diyl | 4 | CH₃ | H |
| 17A | 3-methoxy-1-methyl-1H-pyrazol-4,5-diyl | 4 | CH₃ | CH₃ |
| 18A | 3-methoxy-1-methyl-1H-pyrazol-4,5-diyl | 4 | CH₃ | Cl |
| 19A | 2-methoxy-1-methyl-1H-imidazole-4,5-diyl | 4 | H | H |
| 20A | 2-methoxy-1-methyl-1H-imidazole-4,5-diyl | 4 | H | CH₃ |
| 21A | 2-methoxy-1-methyl-1H-imidazole-4,5-diyl | 4 | H | Cl |
| 22A | 3-methoxy-1-methyl-1H-pyrazol-4,5-diyl | 4 | H | H |
| 23A | 3-methoxy-1-methyl-1H-pyrazol-4,5-diyl | 4 | H | CH₃ |
| 24A | 3-methoxy-1-methyl-1H-pyrazol-4,5-diyl | 4 | H | Cl |

Note:
Column * indicates ring C's point of connection to the adjacent phen-1,4-ylene.

Some embodiments of compounds described herein are selected from the following compounds as listed in Table 11B.

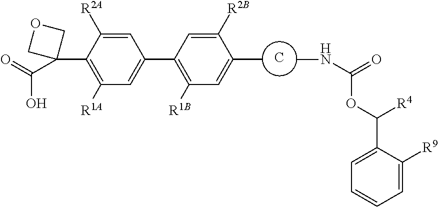

TABLE 11B

| Cmpd # XI- | 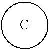 | * | R^{1A} | R^{2A} | R^{1B} | R^{2B} | R⁴ | R⁹ |
|---|---|---|---|---|---|---|---|---|
| 1B | isooxazol-4,5-diyl | 5 | H | H | H | H | CH₃ | H |
| 2B | 1-methyl-pyrazol-4,5-diyl | 4 | H | H | H | H | H | CH₃ |
| 3B | 3-methyl isothiazol-4,5-diyl | 5 | H | H | H | H | CH₃ | H |
| 4B | 3-methyl isothiazol-4,5-diyl | 5 | H | H | H | H | H | Cl |
| 5B | 4-methyl 1,2,3-triazol-1,5-diyl | 1 | H | H | H | H | H | CH₃ |
| 6B | 1,2,3-triazol-4,5-diyl | 5 | H | H | H | H | CH₃ | H |
| 7B | isooxazol-3,4-diyl | 3 | H | H | H | H | CH₃ | H |
| 8B | 3-methyl isooxazol-4,5-diyl | 5 | H | H | H | H | CH₃ | Cl |
| 9B | 5-methyl isooxazol-3,4-diyl | 3 | H | H | H | H | CH₃ | H |
| 10B | 3-methyl isooxazol-4,5-diyl | 5 | H | H | H | H | CH₃ | H |
| 11B | 3-methyl isooxazol-4,5-diyl | 5 | H | H | H | H | H | H |
| 12B | 3-methyl isooxazol-4,5-diyl | 5 | H | H | H | H | H | CH₃ |
| 13B | 5-methyl isothiazol-3,4-diyl | 3 | H | H | H | H | CH₃ | H |
| 14B | 1,2,3-triazol-4,5-diyl | 5 | H | H | H | H | CH₃ | Cl |
| 15B | 1H-pyrazol-4,5-diyl | 4 | H | H | H | H | CH₃ | CH₃ |
| 16B | 1H-pyrazol-4,5-diyl | 4 | H | H | H | H | CH₃ | Cl |
| 17B | isothiazol-4,5-diyl | 5 | H | H | H | H | CH₃ | CH₃ |
| 18B | isothiazol-4,5-diyl | 5 | H | H | H | H | CH₃ | Cl |
| 19B | 1H-pyrazol-4,5-diyl | 4 | H | H | H | H | CH₃ | H |
| 20B | isooxazol-4,5-diyl | 5 | H | H | H | H | CH₃ | Cl |
| 21B | 1-methyl-pyrazol-4,5-diyl | 4 | H | H | H | H | CH₃ | H |
| 22B | 5-methyl isothiazol-3,4-diyl | 3 | H | H | H | H | H | CH₃ |
| 23B | 1-methyl-pyrazol-4,5-diyl | 4 | H | H | H | H | H | H |
| 24B | 5-methyl isothiazol-3,4-diyl | 3 | H | H | H | H | CH₃ | CH₃ |
| 25B | 3-methyl isothiazol-4,5-diyl | 5 | H | H | H | H | H | CH₃ |
| 26B | 3-methyl isothiazol-4,5-diyl | 5 | H | H | H | H | CH₃ | Cl |
| 27B | 3-methyl isothiazol-4,5-diyl | 5 | H | H | H | H | H | H |
| 28B | isooxazol-4,5-diyl | 5 | H | H | H | H | H | CH₃ |
| 29B | 5-methyl isooxazol-3,4-diyl | 3 | H | H | H | H | H | CH₃ |
| 30B | 5-methyl isothiazol-3,4-diyl | 3 | H | H | H | H | H | H |
| 31B | isooxazol-4,5-diyl | 5 | H | H | H | H | H | Cl |
| 32B | isothiazol-4,5-diyl | 5 | H | H | H | H | H | Cl |
| 33B | isooxazol-3,4-diyl | 3 | H | H | H | H | H | H |
| 34B | 1H-pyrazol-4,5-diyl | 4 | H | H | H | H | H | Cl |
| 35B | 5-methyl isooxazol-3,4-diyl | 3 | H | H | H | H | CH₃ | CH₃ |
| 36B | isothiazol-3,4-diyl | 3 | H | H | H | H | CH₃ | H |
| 37B | 5-methyl isothiazol-3,4-diyl | 3 | H | H | H | H | CH₃ | Cl |
| 38B | isooxazol-4,5-diyl | 5 | H | H | H | H | H | H |
| 39B | 5-methyl isooxazol-3,4-diyl | 3 | H | H | H | H | H | H |
| 40B | isothiazol-4,5-diyl | 5 | H | H | H | H | H | H |
| 41B | isooxazol-4,5-diyl | 5 | H | H | H | H | CH₃ | CH₃ |
| 42B | 5-methyl isooxazol-3,4-diyl | 3 | H | H | H | H | H | Cl |
| 43B | 5-methyl isooxazol-3,4-diyl | 3 | H | H | H | H | CH₃ | Cl |
| 44B | 1-methyl-1,2,3-triazol-4,5-diyl | 4 | H | H | H | H | CH₃ | Cl |
| 45B | 1-methyl-1,2,3-triazol-4,5-diyl | 4 | H | H | H | H | H | H |
| 46B | 4-methyl 1,2,3-triazol-1,5-diyl | 1 | H | H | H | H | H | Cl |
| 47B | 1,2,3-triazol-4,5-diyl | 5 | H | H | H | H | H | CH₃ |
| 48B | 1-methyl-1,2,3-triazol-4,5-diyl | 4 | H | H | H | H | H | CH₃ |
| 49B | 1,2,3-triazol-4,5-diyl | 5 | H | H | H | H | H | Cl |
| 50B | 1-methyl-1,2,3-triazol-4,5-diyl | 4 | H | H | H | H | H | Cl |
| 51B | 1,2,3-triazol-1,5-diyl | 1 | H | H | H | H | H | Cl |
| 52B | 1H-pyrazol-4,5-diyl | 4 | H | H | H | H | H | H |
| 53B | 1-methyl-pyrazol-4,5-diyl | 4 | H | H | H | H | H | Cl |
| 54B | 1,2,3-triazol-1,5-diyl | 1 | H | H | H | H | CH₃ | Cl |
| 55B | 1,2,3-triazol-4,5-diyl | 5 | H | H | H | H | H | H |
| 56B | isooxazol-3,4-diyl | 3 | H | H | H | H | CH₃ | Cl |
| 57B | 1-methyl-1,2,3-triazol-4,5-diyl | 4 | H | H | H | H | CH₃ | H |
| 58B | 4-methyl 1,2,3-triazol-1,5-diyl | 1 | H | H | H | H | CH₃ | Cl |
| 59B | 3-methyl isooxazol-4,5-diyl | 5 | H | H | H | H | CH₃ | CH₃ |
| 60B | 5-methyl isothiazol-3,4-diyl | 3 | H | H | H | H | H | Cl |
| 61B | 1-methyl-1,2,3-triazol-4,5-diyl | 4 | H | H | H | H | CH₃ | CH₃ |
| 62B | 4-methyl 1,2,3-triazol-1,5-diyl | 1 | H | H | H | H | H | H |
| 63B | 3-methyl isooxazol-4,5-diyl | 5 | H | H | H | H | H | Cl |
| 64B | 1-methyl-pyrazol-4,5-diyl | 4 | H | H | H | H | CH₃ | CH₃ |
| 65B | 3-methyl isothiazol-4,5-diyl | 5 | H | H | H | H | CH₃ | CH₃ |
| 66B | 4-methyl 1,2,3-triazol-1,5-diyl | 1 | H | H | H | H | CH₃ | CH₃ |
| 67B | isothiazol-4,5-diyl | 5 | H | H | H | H | CH₃ | H |
| 68B | 1-methyl-pyrazol-4,5-diyl | 4 | H | H | H | H | CH₃ | Cl |
| 69B | 1,2,3-triazol-1,5-diyl | 1 | H | H | H | H | CH₃ | H |
| 70B | isooxazol-3,4-diyl | 3 | H | H | H | H | CH₃ | CH₃ |
| 71B | isothiazol-3,4-diyl | 3 | H | H | H | H | H | CH₃ |
| 72B | isothiazol-3,4-diyl | 3 | H | H | H | H | H | Cl |
| 73B | 1,2,3-triazol-4,5-diyl | 5 | H | H | H | H | CH₃ | CH₃ |
| 74B | 1,2,3-triazol-1,5-diyl | 1 | H | H | H | H | H | H |
| 75B | 1,2,3-triazol-1,5-diyl | 1 | H | H | H | H | H | CH₃ |
| 76B | isothiazol-3,4-diyl | 3 | H | H | H | H | CH₃ | Cl |
| 77B | isothiazol-3,4-diyl | 3 | H | H | H | H | H | H |
| 78B | isothiazol-3,4-diyl | 3 | H | H | H | H | CH₃ | CH₃ |
| 79B | 1H-pyrazol-4,5-diyl | 4 | H | H | H | H | H | CH₃ |
| 80B | isooxazol-3,4-diyl | 3 | H | H | H | H | H | CH₃ |
| 81B | isooxazol-3,4-diyl | 3 | H | H | H | H | H | Cl |
| 82B | 1,2,3-triazol-1,5-diyl | 1 | H | H | H | H | CH₃ | CH₃ |
| 83B | 4-methyl 1,2,3-triazol-1,5-diyl | 1 | H | H | H | H | CH₃ | H |
| 84B | isothiazol-4,5-diyl | 5 | H | H | H | H | H | CH₃ |
| 85B | 4-methyl 1,2,3-triazol-1,5-diyl | 1 | F | H | H | H | H | CH₃ |
| 86B | 3-methyl isooxazol-4,5-diyl | 5 | F | H | H | H | CH₃ | H |
| 87B | 3-methyl isothiazol-4,5-diyl | 5 | H | H | F | H | CH₃ | H |
| 88B | 3-methyl isothiazol-4,5-diyl | 5 | F | H | H | H | CH₃ | H |
| 89B | 3-methyl isooxazol-4,5-diyl | 5 | H | H | H | F | H | CH₃ |
| 90B | 5-methyl isooxazol-3,4-diyl | 3 | H | H | H | F | CH₃ | H |
| 91B | 5-methyl isooxazol-3,4-diyl | 3 | F | H | H | H | H | Cl |
| 92B | 3-methyl isooxazol-4,5-diyl | 5 | H | H | H | F | CH₃ | H |
| 93B | 3-methyl isooxazol-4,5-diyl | 5 | H | H | F | H | CH₃ | H |
| 94B | 3-methyl isooxazol-4,5-diyl | 5 | H | H | F | H | CH₃ | CH₃ |
| 95B | isothiazol-4,5-diyl | 5 | H | H | H | F | H | CH₃ |
| 96B | isothiazol-4,5-diyl | 5 | H | H | F | H | H | CH₃ |
| 97B | isothiazol-4,5-diyl | 5 | F | H | H | H | H | CH₃ |
| 98B | 1,2,3-triazol-4,5-diyl | 5 | H | H | H | F | H | CH₃ |
| 99B | isooxazol-3,4-diyl | 3 | H | H | F | H | H | CH₃ |
| 100B | 5-methyl isooxazol-3,4-diyl | 3 | F | H | H | H | H | CH₃ |
| 101B | 5-methyl isothiazol-3,4-diyl | 3 | F | H | H | H | CH₃ | H |
| 102B | 5-methyl isooxazol-3,4-diyl | 3 | H | H | F | H | H | H |
| 103B | 1,2,3-triazol-4,5-diyl | 5 | H | H | F | H | H | H |
| 104B | isooxazol-3,4-diyl | 3 | H | H | F | H | H | H |
| 105B | isooxazol-3,4-diyl | 3 | H | H | H | F | H | H |
| 106B | 5-methyl isooxazol-3,4-diyl | 3 | F | H | H | H | H | H |
| 107B | 3-methyl isooxazol-4,5-diyl | 5 | H | H | H | F | H | H |
| 108B | 5-methyl isooxazol-3,4-diyl | 3 | H | H | F | H | CH₃ | H |
| 109B | 5-methyl isooxazol-3,4-diyl | 3 | F | H | H | H | CH₃ | H |
| 110B | 1-methyl-1,2,3-triazol-4,5-diyl | 4 | H | H | F | H | CH₃ | H |
| 111B | 4-methyl 1,2,3-triazol-1,5-diyl | 1 | H | H | H | F | CH₃ | H |
| 112B | 4-methyl 1,2,3-triazol-1,5-diyl | 1 | H | H | F | H | CH₃ | H |
| 113B | 4-methyl 1,2,3-triazol-1,5-diyl | 1 | F | H | H | H | CH₃ | H |
| 114B | 1,2,3-triazol-4,5-diyl | 5 | H | H | F | H | H | CH₃ |
| 115B | 1,2,3-triazol-4,5-diyl | 5 | F | H | H | H | H | CH₃ |
| 116B | isooxazol-4,5-diyl | 5 | F | H | H | H | CH₃ | Cl |
| 117B | isooxazol-3,4-diyl | 3 | H | H | F | H | CH₃ | H |
| 118B | isooxazol-4,5-diyl | 5 | F | H | H | H | CH₃ | H |
| 119B | isooxazol-4,5-diyl | 5 | F | H | H | H | H | H |
| 120B | isooxazol-4,5-diyl | 5 | H | H | H | F | H | CH₃ |
| 121B | isooxazol-4,5-diyl | 5 | H | H | F | H | H | CH₃ |
| 122B | isooxazol-4,5-diyl | 5 | F | H | H | H | H | CH₃ |
| 123B | isothiazol-3,4-diyl | 3 | F | H | H | H | CH₃ | H |
| 124B | 1,2,3-triazol-4,5-diyl | 5 | H | H | F | H | H | Cl |
| 125B | 1,2,3-triazol-4,5-diyl | 5 | F | H | H | H | H | Cl |
| 126B | 3-methyl isooxazol-4,5-diyl | 5 | H | H | H | F | H | Cl |
| 127B | 3-methyl isooxazol-4,5-diyl | 5 | H | H | F | H | H | Cl |
| 128B | isothiazol-4,5-diyl | 5 | H | H | H | F | H | Cl |
| 129B | isothiazol-4,5-diyl | 5 | F | H | H | H | H | Cl |
| 130B | 1H-pyrazol-4,5-diyl | 4 | H | H | F | H | H | Cl |
| 131B | isooxazol-3,4-diyl | 3 | H | H | F | H | H | Cl |
| 132B | 1H-pyrazol-4,5-diyl | 4 | H | H | H | F | H | CH₃ |
| 133B | 1H-pyrazol-4,5-diyl | 4 | H | H | F | H | H | CH₃ |
| 134B | 1H-pyrazol-4,5-diyl | 4 | F | H | H | H | H | CH₃ |
| 135B | 1H-pyrazol-4,5-diyl | 4 | H | H | H | F | H | Cl |
| 136B | isooxazol-4,5-diyl | 5 | H | H | H | F | CH₃ | Cl |
| 137B | 1,2,3-triazol-4,5-diyl | 5 | H | H | F | H | CH₃ | Cl |
| 138B | 4-methyl 1,2,3-triazol-1,5-diyl | 1 | F | H | H | H | CH₃ | Cl |
| 139B | 1-methyl-pyrazol-4,5-diyl | 4 | F | H | H | H | CH₃ | Cl |
| 140B | isothiazol-4,5-diyl | 5 | H | H | H | F | CH₃ | Cl |
| 141B | 1,2,3-triazol-4,5-diyl | 5 | F | H | H | H | CH₃ | Cl |
| 142B | isooxazol-3,4-diyl | 3 | F | H | H | H | CH₃ | Cl |
| 143B | isothiazol-3,4-diyl | 3 | F | H | H | H | CH₃ | Cl |
| 144B | 4-methyl 1,2,3-triazol-1,5-diyl | 1 | F | H | H | H | H | H |
| 145B | 4-methyl 1,2,3-triazol-1,5-diyl | 1 | F | H | H | H | H | Cl |
| 146B | 3-methyl isothiazol-4,5-diyl | 5 | H | H | H | F | CH₃ | H |
| 147B | 1-methyl-1,2,3-triazol-4,5-diyl | 4 | H | H | H | F | CH₃ | H |
| 148B | 1-methyl-1,2,3-triazol-4,5-diyl | 4 | F | H | H | H | CH₃ | H |
| 149B | 1-methyl-pyrazol-4,5-diyl | 4 | H | H | H | F | CH₃ | H |
| 150B | 1-methyl-pyrazol-4,5-diyl | 4 | H | H | F | H | CH₃ | H |
| 151B | 1-methyl-pyrazol-4,5-diyl | 4 | F | H | H | H | CH₃ | H |
| 152B | 3-methyl isooxazol-4,5-diyl | 5 | H | H | F | H | H | CH₃ |
| 153B | 3-methyl isooxazol-4,5-diyl | 5 | F | H | H | H | H | CH₃ |
| 154B | 1-methyl-1,2,3-triazol-4,5-diyl | 4 | F | H | H | H | H | CH₃ |
| 155B | 1-methyl-1,2,3-triazol-4,5-diyl | 4 | F | H | H | H | H | Cl |
| 156B | 1-methyl-1,2,3-triazol-4,5-diyl | 4 | F | H | H | H | CH₃ | CH₃ |
| 157B | 4-methyl 1,2,3-triazol-1,5-diyl | 1 | F | H | H | H | CH₃ | CH₃ |
| 158B | 1-methyl-pyrazol-4,5-diyl | 4 | F | H | H | H | H | CH₃ |
| 159B | 1-methyl-pyrazol-4,5-diyl | 4 | F | H | H | H | H | Cl |
| 160B | 5-methyl isooxazol-3,4-diyl | 3 | H | H | F | H | CH₃ | CH₃ |
| 161B | 4-methyl 1,2,3-triazol-1,5-diyl | 1 | H | H | H | F | CH₃ | CH₃ |
| 162B | isooxazol-4,5-diyl | 5 | H | H | F | H | CH₃ | CH₃ |
| 163B | isothiazol-4,5-diyl | 5 | H | H | H | F | CH₃ | CH₃ |
| 164B | isothiazol-4,5-diyl | 5 | H | H | F | H | CH₃ | CH₃ |
| 165B | isothiazol-4,5-diyl | 5 | F | H | H | H | CH₃ | CH₃ |
| 166B | 1,2,3-triazol-4,5-diyl | 5 | H | H | H | F | CH₃ | CH₃ |
| 167B | 1,2,3-triazol-4,5-diyl | 5 | H | H | F | H | CH₃ | CH₃ |
| 168B | isothiazol-3,4-diyl | 3 | H | H | F | H | CH₃ | CH₃ |
| 169B | 5-methyl isooxazol-3,4-diyl | 3 | F | H | H | H | CH₃ | CH₃ |
| 170B | 3-methyl isooxazol-4,5-diyl | 5 | H | H | H | F | CH₃ | CH₃ |
| 171B | 3-methyl isooxazol-4,5-diyl | 5 | F | H | H | H | CH₃ | CH₃ |
| 172B | isooxazol-4,5-diyl | 5 | F | H | H | H | CH₃ | CH₃ |
| 173B | isooxazol-3,4-diyl | 3 | H | H | H | F | CH₃ | H |
| 174B | isooxazol-3,4-diyl | 3 | F | H | H | H | CH₃ | H |
| 175B | isothiazol-3,4-diyl | 3 | H | H | F | H | CH₃ | H |
| 176B | isothiazol-3,4-diyl | 3 | H | H | H | F | CH₃ | H |
| 177B | isothiazol-4,5-diyl | 5 | H | H | F | H | CH₃ | H |
| 178B | isothiazol-4,5-diyl | 5 | H | H | H | F | CH₃ | H |
| 179B | isothiazol-4,5-diyl | 5 | F | H | H | H | CH₃ | H |
| 180B | 1,2,3-triazol-4,5-diyl | 5 | H | H | F | H | CH₃ | H |
| 181B | 1,2,3-triazol-4,5-diyl | 5 | H | H | H | F | CH₃ | H |
| 182B | 1,2,3-triazol-4,5-diyl | 5 | F | H | H | H | CH₃ | H |
| 183B | 1H-pyrazol-4,5-diyl | 4 | H | H | F | H | CH₃ | H |
| 184B | 1H-pyrazol-4,5-diyl | 4 | H | H | H | F | CH₃ | H |
| 185B | 1H-pyrazol-4,5-diyl | 4 | F | H | H | H | CH₃ | H |
| 186B | 5-methyl isooxazol-3,4-diyl | 3 | H | H | F | H | CH₃ | Cl |
| 187B | 4-methyl 1,2,3-triazol-1,5-diyl | 1 | H | H | H | F | CH₃ | Cl |
| 188B | isooxazol-4,5-diyl | 5 | H | H | F | H | CH₃ | Cl |
| 189B | isothiazol-4,5-diyl | 5 | H | H | F | H | CH₃ | Cl |
| 190B | isothiazol-4,5-diyl | 5 | F | H | H | H | CH₃ | Cl |
| 191B | 1,2,3-triazol-4,5-diyl | 5 | H | H | H | F | CH₃ | Cl |
| 192B | 5-methyl isothiazol-3,4-diyl | 3 | F | H | H | H | CH₃ | Cl |
| 193B | isooxazol-3,4-diyl | 3 | H | H | F | H | CH₃ | Cl |
| 194B | isooxazol-3,4-diyl | 3 | H | H | H | F | CH₃ | Cl |
| 195 | isothiazol-3,4-diyl | 3 | H | H | F | H | CH₃ | Cl |
| 196F | isothiazol-3,4-diyl | 3 | H | H | H | F | CH₃ | Cl |
| 197B | 5-methyl isooxazol-3,4-diyl | 3 | F | H | H | H | CH₃ | Cl |
| 198B | 1-methyl-1,2,3-triazol-4,5-diyl | 4 | F | H | H | H | CH₃ | Cl |
| 199B | 3-methyl isooxazol-4,5-diyl | 5 | H | H | F | H | CH₃ | Cl |
| 200B | 3-methyl isooxazol-4,5-diyl | 5 | H | H | H | F | CH₃ | Cl |
| 201B | 3-methyl isooxazol-4,5-diyl | 5 | F | H | H | H | CH₃ | Cl |
| 202B | 1,2,3-triazol-1,5-diyl | 1 | H | H | H | F | CH₃ | Cl |
| 203B | 1,2,3-triazol-1,5-diyl | 1 | H | H | F | H | CH₃ | Cl |
| 204B | 1,2,3-triazol-1,5-diyl | 1 | F | H | H | H | CH₃ | Cl |
| 205B | isooxazol-3,4-diyl | 3 | F | H | H | H | CH₃ | CH₃ |
| 206B | isothiazol-3,4-diyl | 3 | H | H | F | H | H | CH₃ |
| 207B | isothiazol-3,4-diyl | 3 | H | H | H | F | H | CH₃ |
| 208B | isothiazol-3,4-diyl | 3 | F | H | H | H | H | CH₃ |
| 209B | isothiazol-3,4-diyl | 3 | H | H | F | H | H | Cl |
| 210B | isothiazol-3,4-diyl | 3 | H | H | H | F | H | Cl |
| 211B | isothiazol-3,4-diyl | 3 | F | H | H | H | H | Cl |
| 212B | 1,2,3-triazol-4,5-diyl | 5 | F | H | H | H | CH₃ | CH₃ |
| 213B | 1,2,3-triazol-1,5-diyl | 1 | H | H | H | F | H | H |
| 214B | 1,2,3-triazol-1,5-diyl | 1 | H | H | F | H | H | H |
| 215B | 1,2,3-triazol-1,5-diyl | 1 | F | H | H | H | H | H |
| 216B | 1,2,3-triazol-1,5-diyl | 1 | H | H | H | F | H | CH₃ |
| 217B | 1,2,3-triazol-1,5-diyl | 1 | H | H | F | H | H | CH₃ |
| 218B | 1,2,3-triazol-1,5-diyl | 1 | F | H | H | H | H | CH₃ |
| 219B | 1H-pyrazol-4,5-diyl | 4 | H | H | F | H | H | H |
| 220B | 1H-pyrazol-4,5-diyl | 4 | F | H | H | H | H | H |
| 221B | 1,2,3-triazol-1,5-diyl | 1 | H | H | F | H | CH₃ | H |
| 222B | 1,2,3-triazol-1,5-diyl | 1 | H | H | H | F | CH₃ | H |
| 223B | 1,2,3-triazol-1,5-diyl | 1 | F | H | H | H | CH₃ | H |
| 224B | 1H-pyrazol-4,5-diyl | 4 | H | H | H | F | CH₃ | Cl |
| 225B | 1H-pyrazol-4,5-diyl | 4 | H | H | F | H | CH₃ | Cl |
| 226B | 1H-pyrazol-4,5-diyl | 4 | F | H | H | H | CH₃ | Cl |
| 227B | isooxazol-4,5-diyl | 5 | F | H | H | H | H | Cl |
| 228B | 3-methyl isooxazol-4,5-diyl | 5 | F | H | H | H | H | Cl |
| 229B | isooxazol-4,5-diyl | 5 | H | H | H | F | CH₃ | H |
| 230B | isooxazol-4,5-diyl | 5 | H | H | F | H | CH₃ | H |
| 231B | 4-methyl 1,2,3-triazol-1,5-diyl | 1 | H | H | H | F | H | H |
| 232B | isothiazol-4,5-diyl | 5 | H | H | H | F | H | H |
| 233B | isothiazol-4,5-diyl | 5 | H | H | F | H | H | H |
| 234B | isothiazol-4,5-diyl | 5 | F | H | H | H | H | H |
| 235B | 1-methyl-pyrazol-4,5-diyl | 4 | F | H | H | H | H | H |
| 236B | isooxazol-3,4-diyl | 3 | F | H | H | H | H | H |
| 237B | isothiazol-3,4-diyl | 3 | H | H | F | H | H | H |
| 238B | 5-methyl isothiazol-3,4-diyl | 3 | F | H | H | H | H | H |
| 239B | isothiazol-3,4-diyl | 3 | H | H | H | F | H | H |
| 240B | isothiazol-3,4-diyl | 3 | F | H | H | H | H | H |
| 241B | 1H-pyrazol-4,5-diyl | 4 | F | H | H | H | H | Cl |
| 242B | 3-methyl isooxazol-4,5-diyl | 5 | H | H | F | H | H | H |
| 243B | 3-methyl isooxazol-4,5-diyl | 5 | F | H | H | H | H | H |
| 244B | isooxazol-4,5-diyl | 5 | H | H | H | F | H | H |
| 245B | isooxazol-4,5-diyl | 5 | H | H | F | H | H | H |
| 246B | 1,2,3-triazol-4,5-diyl | 5 | F | H | H | H | H | H |
| 247B | 1H-pyrazol-4,5-diyl | 4 | H | H | H | F | H | H |
| 248B | 1-methyl-1,2,3-triazol-4,5-diyl | 4 | F | H | H | H | H | H |
| 249B | 5-methyl isooxazol-3,4-diyl | 3 | H | H | F | H | H | CH₃ |
| 250B | 4-methyl 1,2,3-triazol-1,5-diyl | 1 | H | H | H | F | H | CH₃ |
| 251B | 5-methyl isothiazol-3,4-diyl | 3 | F | H | H | H | H | CH₃ |
| 252B | isooxazol-3,4-diyl | 3 | H | H | H | F | H | CH₃ |
| 253B | isooxazol-3,4-diyl | 3 | F | H | H | H | H | CH₃ |
| 254B | 1H-pyrazol-4,5-diyl | 4 | H | H | H | F | CH₃ | CH₃ |
| 255B | 1H-pyrazol-4,5-diyl | 4 | H | H | F | H | CH₃ | CH₃ |
| 256B | 1H-pyrazol-4,5-diyl | 4 | F | H | H | H | CH₃ | CH₃ |
| 257B | 5-methyl isothiazol-3,4-diyl | 3 | F | H | H | H | CH₃ | CH₃ |
| 258B | isothiazol-3,4-diyl | 3 | H | H | H | F | CH₃ | CH₃ |
| 259B | isothiazol-3,4-diyl | 3 | F | H | H | H | CH₃ | CH₃ |
| 260B | 1-methyl-pyrazol-4,5-diyl | 4 | F | H | H | H | CH₃ | CH₃ |
| 261B | 5-methyl isooxazol-3,4-diyl | 3 | H | H | F | H | H | Cl |
| 262B | 4-methyl 1,2,3-triazol-1,5-diyl | 1 | H | H | H | F | H | Cl |
| 263B | isooxazol-4,5-diyl | 5 | H | H | H | F | H | Cl |
| 264B | isooxazol-4,5-diyl | 5 | H | H | F | H | H | Cl |
| 265B | isothiazol-4,5-diyl | 5 | H | H | F | H | H | Cl |
| 266B | 1,2,3-triazol-4,5-diyl | 5 | H | H | H | F | H | Cl |
| 267B | 5-methyl isothiazol-3,4-diyl | 3 | F | H | H | H | H | Cl |
| 268B | isooxazol-3,4-diyl | 3 | H | H | H | F | H | Cl |
| 269B | isooxazol-3,4-diyl | 3 | F | H | H | H | H | Cl |
| 270B | 1,2,3-triazol-1,5-diyl | 1 | H | H | H | F | H | Cl |
| 271B | 1,2,3-triazol-1,5-diyl | 1 | H | H | F | H | H | Cl |
| 272B | 1,2,3-triazol-1,5-diyl | 1 | F | H | H | H | H | Cl |
| 273B | 1,2,3-triazol-1,5-diyl | 1 | H | H | F | H | CH₃ | CH₃ |
| 274B | 1,2,3-triazol-1,5-diyl | 1 | F | H | H | H | CH₃ | CH₃ |
| 275B | 5-methyl isothiazol-3,4-diyl | 3 | H | H | H | F | CH₃ | H |
| 276B | isooxazol-4,5-diyl | 5 | H | H | H | F | CH₃ | CH₃ |
| 277B | isooxazol-3,4-diyl | 3 | H | H | H | F | CH₃ | CH₃ |
| 278B | 1,2,3-triazol-4,5-diyl | 5 | H | H | H | F | H | H |
| 279B | 1,2,3-triazol-1,5-diyl | 1 | H | H | H | F | CH₃ | CH₃ |
| 280B | 5-methyl isothiazol-3,4-diyl | 3 | H | H | F | H | CH₃ | H |
| 281B | isooxazol-3,4-diyl | 3 | H | H | F | H | CH₃ | CH₃ |
| 282B | 5-methyl isooxazol-3,4-diyl | 3 | F | F | H | H | CH₃ | H |
| 283B | 1,2,3-triazol-4,5-diyl | 5 | F | F | H | H | H | CH₃ |
| 284B | 1,2,3-triazol-4,5-diyl | 5 | F | F | H | H | H | Cl |
| 285B | 1,2,3-triazol-1,5-diyl | 1 | F | F | H | H | H | Cl |
| 286B | 1,2,3-triazol-1,5-diyl | 1 | F | F | H | H | CH₃ | Cl |
| 287B | 1,2,3-triazol-4,5-diyl | 5 | F | F | H | H | CH₃ | Cl |
| 288B | isooxazol-3,4-diyl | 3 | F | F | H | H | CH₃ | Cl |
| 289B | 1,2,3-triazol-4,5-diyl | 5 | F | F | H | H | CH₃ | H |
| 290B | isooxazol-3,4-diyl | 3 | F | F | H | H | CH₃ | CH₃ |
| 291B | isothiazol-3,4-diyl | 3 | F | F | H | H | H | CH₃ |
| 292B | isothiazol-3,4-diyl | 3 | F | F | H | H | H | Cl |
| 293B | 1,2,3-triazol-4,5-diyl | 5 | F | F | H | H | H | H |
| 294B | 1,2,3-triazol-4,5-diyl | 5 | F | F | H | H | CH₃ | CH₃ |
| 295B | 1,2,3-triazol-1,5-diyl | 1 | F | F | H | H | H | H |
| 296B | 1,2,3-triazol-1,5-diyl | 1 | F | F | H | H | H | CH₃ |
| 297B | isooxazol-3,4-diyl | 3 | F | F | H | H | H | H |
| 298B | isooxazol-3,4-diyl | 3 | F | F | H | H | H | CH₃ |
| 299B | isooxazol-3,4-diyl | 3 | F | F | H | H | H | Cl |
| 300B | 1,2,3-triazol-1,5-diyl | 1 | F | F | H | H | CH₃ | CH₃ |
| 301B | 3-methyl isooxazol-4,5-diyl | 5 | F | F | H | H | CH₃ | Cl |
| 302B | isooxazol-4,5-diyl | 5 | F | F | H | H | CH₃ | H |
| 303B | isothiazol-4,5-diyl | 5 | F | F | H | H | CH₃ | H |
| 304B | 1H-pyrazol-4,5-diyl | 4 | F | F | H | H | CH₃ | H |
| 305B | isooxazol-4,5-diyl | 5 | F | F | H | H | H | CH₃ |
| 306B | 3-methyl isooxazol-4,5-diyl | 5 | F | F | H | H | CH₃ | H |
| 307B | 3-methyl isooxazol-4,5-diyl | 5 | F | F | H | H | H | H |
| 308B | 3-methyl isooxazol-4,5-diyl | 5 | F | F | H | H | H | CH₃ |
| 309B | 5-methyl isothiazol-3,4-diyl | 3 | F | F | H | H | CH₃ | H |
| 310B | isooxazol-3,4-diyl | 3 | F | F | H | H | CH₃ | H |
| 311B | isothiazol-3,4-diyl | 3 | F | F | H | H | CH₃ | H |
| 312B | 3-methyl isooxazol-4,5-diyl | 5 | H | H | F | F | CH₃ | Cl |
| 313B | 5-methyl isooxazol-3,4-diyl | 3 | H | H | F | F | CH₃ | H |
| 314B | isooxazol-4,5-diyl | 5 | H | H | F | F | CH₃ | H |
| 315B | isothiazol-4,5-diyl | 5 | H | H | F | F | CH₃ | H |
| 316B | 1H-pyrazol-4,5-diyl | 4 | H | H | F | F | CH₃ | H |
| 317B | isooxazol-4,5-diyl | 5 | H | H | F | F | H | CH₃ |
| 318B | 3-methyl isooxazol-4,5-diyl | 5 | H | H | F | F | CH₃ | H |
| 319B | 3-methyl isooxazol-4,5-diyl | 5 | H | H | F | F | H | H |
| 320B | 3-methyl isooxazol-4,5-diyl | 5 | H | H | F | F | H | CH₃ |
| 321B | isooxazol-4,5-diyl | 5 | H | H | F | F | H | H |
| 322B | 4-methyl 1,2,3-triazol-1,5-diyl | 1 | H | H | F | F | CH₃ | H |
| 323B | 1,2,3-triazol-4,5-diyl | 5 | H | H | F | F | CH₃ | H |
| 324B | isooxazol-3,4-diyl | 3 | H | H | F | F | CH₃ | H |
| 325B | isothiazol-3,4-diyl | 3 | H | H | F | F | CH₃ | H |
| 326B | isooxazol-4,5-diyl | 5 | F | F | H | H | H | Cl |
| 327B | 1-methyl-1,2,3-triazol-4,5-diyl | 4 | F | F | H | H | CH₃ | H |
| 328B | 1-methyl-pyrazol-4,5-diyl | 4 | F | F | H | H | CH₃ | H |
| 329B | 3-methyl isooxazol-4,5-diyl | 5 | F | F | H | H | H | Cl |
| 330B | isooxazol-4,5-diyl | 5 | F | F | H | H | CH₃ | Cl |
| 331B | 1H-pyrazol-4,5-diyl | 4 | F | F | H | H | CH₃ | Cl |
| 332B | 1H-pyrazol-4,5-diyl | 4 | F | F | H | H | H | CH₃ |
| 333B | 1H-pyrazol-4,5-diyl | 4 | F | F | H | H | H | Cl |
| 334B | isooxazol-4,5-diyl | 5 | F | F | H | H | H | H |
| 335B | 4-methyl 1,2,3-triazol-1,5-diyl | 1 | F | F | H | H | CH₃ | H |
| 336B | isothiazol-3,4-diyl | 3 | F | F | H | H | CH₃ | Cl |
| 337B | isooxazol-4,5-diyl | 5 | F | F | H | H | CH₃ | CH₃ |
| 338B | isothiazol-3,4-diyl | 3 | F | F | H | H | H | H |
| 339B | isothiazol-3,4-diyl | 3 | F | F | H | H | CH₃ | CH₃ |

Note:
column * indicates ring C's point of connection to the adjacent optionally substituted phen-1,4-ylene.

Some embodiments of compounds described herein are selected from the following compounds as listed in Table 11C.

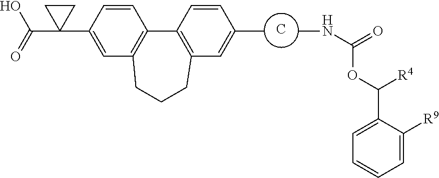

TABLE 11C

| Cmpd # XI- | 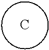 | * | R⁴ | R⁹ |
|---|---|---|---|---|
| 1C | 3-methyl isooxazol-4,5-diyl | 5 | CH₃ | H |
| 2C | 3-methyl isooxazol-4,5-diyl | 5 | CH₃ | CH₃ |
| 3C | 3-methyl isooxazol-4,5-diyl | 5 | CH₃ | Cl |
| 4C | 3-methyl isothiazole-4,5-diyl | 5 | CH₃ | H |
| 5C | 3-methyl isothiazole-4,5-diyl | 5 | CH₃ | CH₃ |
| 6C | 3-methyl isothiazole-4,5-diyl | 5 | CH₃ | Cl |
| 7C | 3-methyl-1H-pyrazol-4,5-diyl | 5 | CH₃ | H |
| 8C | 3-methyl-1H-pyrazol-4,5-diyl | 5 | CH₃ | CH₃ |
| 9C | 3-methyl-1H-pyrazol-4,5-diyl | 5 | CH₃ | Cl |
| 10C | 3-methyl isooxazol-4,5-diyl | 5 | H | H |
| 11C | 3-methyl isooxazol-4,5-diyl | 5 | H | CH₃ |
| 12C | 3-methyl isooxazol-4,5-diyl | 5 | H | Cl |
| 13C | 3-methyl isothiazole-4,5-diyl | 5 | H | H |
| 14C | 3-methyl isothiazole-4,5-diyl | 5 | H | CH₃ |
| 15C | 3-methyl isothiazole-4,5-diyl | 5 | H | Cl |
| 16B | 3-methyl-1H-pyrazol-4,5-diyl | 5 | H | H |
| 17B | 3-methyl-1H-pyrazol-4,5-diyl | 5 | H | CH₃ |
| 18B | 3-methyl-1H-pyrazol-4,5-diyl | 5 | H | Cl |

Note:
column * indicates ring C's point of connection to the adjacent polycyclic moiety.

Some embodiments of compounds described herein are selected from the following compounds as listed in Table 11D.

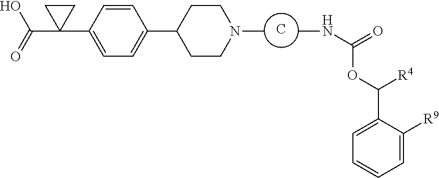

TABLE 11D

| Cmpd # XI- | 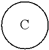 | * | R⁴ | R⁹ |
|---|---|---|---|---|
| 1D | 3-methyl isothiazole-4,5-diyl | 5 | CH₃ | H |
| 2D | 3-methyl isothiazole-4,5-diyl | 5 | CH₃ | CH₃ |
| 3D | 3-methyl isothiazole-4,5-diyl | 5 | CH₃ | Cl |
| 4D | 3-methyl isothiazole-4,5-diyl | 5 | H | H |
| 5D | 3-methyl isothiazole-4,5-diyl | 5 | H | CH₃ |
| 6D | 3-methyl isothiazole-4,5-diyl | 5 | H | Cl |

Note:
column * indicates ring C's point of connection to the adjacent piperidine moiety.

In some embodiments, compounds of Formula (XII) are also represented by Formula (XII-A) and are selected from the following compounds as listed in Table 12A.

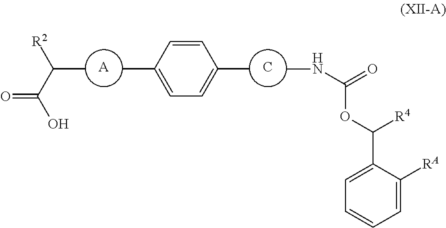

TABLE 12A

| Cmpd # XII- | R² | 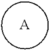 | 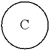 | * | R⁴ | R^{A} |
|---|---|---|---|---|---|---|
| 1A | cyclopropanyl | 1,4-phenylene | isooxazol-3,4-diyl | 3 | methyl | phenyl |
| 2A | oxetan-3-yl | 1,4-phenylene | 3-methyl isooxazol-4,5-diyl | 5 | H | 2-chloro-phenyl |
| 3A | cyclobutanyl | 1,4-phenylene | 1,2,3-triazol-4,5-diyl | 5 | methyl | phenyl |
| 4A | cyclohexanyl | 1,4-phenylene | 1H-pyrazol-4,5-diyl | 4 | methyl | phenyl |
| 5A | oxetan-3-yl | 1,4-phenylene | 3-methyl isooxazol-4,5-diyl | 5 | H | phenyl |
| 6A | cyclopropanyl | 1,4-phenylene | isooxazol-3,4-diyl | 3 | methyl | 2-chloro-phenyl |
| 7A | cyclobutanyl | 1,4-phenylene | 1,2,3-triazol-4,5-diyl | 5 | methyl | 2-chloro-phenyl |
| 8A | cyclohexanyl | 1,4-phenylene | 1H-pyrazol-4,5-diyl | 4 | methyl | 2-chloro-phenyl |
| 9A | cyclopentanyl | 1,4-phenylene | 3-methyl isooxazol-4,5-diyl | 5 | methyl | 2-chloro-phenyl |
| 10A | oxetan-3-yl | 1,4-phenylene | 3-methyl isooxazol-4,5-diyl | 5 | methyl | 2-methyl-phenyl |
| 11A | oxetan-3-yl | 1,4-phenylene | 3-methyl isooxazol-4,5-diyl | 5 | methyl | 2-chloro-phenyl |
| 12A | oxetan-3-yl | 1,4-phenylene | isooxazol-4,5-diyl | 5 | methyl | phenyl |
| 13A | cyclopropanyl | 1,4-phenylene | 5-methyl isooxazol-3,4-diyl | 3 | H | 2-chloro-phenyl |
| 14A | cyclobutanyl | 1,4-phenylene | 1-methyl-1,2,3-triazol-4,5-diyl | 4 | H | 2-chloro-phenyl |
| 15A | oxetan-3-yl | 1,4-phenylene | isooxazol-3,4-diyl | 3 | H | phenyl |
| 16A | oxetan-3-yl | 1,4-phenylene | isooxazol-3,4-diyl | 3 | H | 2-methyl-phenyl |
| 17A | cyclopropanyl | 1,4-phenylene | 3-methyl isooxazol-4,5-diyl | 5 | methyl | phenyl |
| 18A | oxetan-3-yl | 1,4-phenylene | isooxazol-3,4-diyl | 3 | H | 2-chloro-phenyl |
| 19A | oxetan-3-yl | 1,4-phenylene | isothiazol-3,4-diyl | 3 | H | phenyl |
| 20A | oxetan-3-yl | 1,4-phenylene | isothiazol-3,4-diyl | 3 | H | 2-chloro-phenyl |
| 21A | oxetan-3-yl | 1,4-phenylene | 1,2,3-triazol-4,5-diyl | 5 | H | 2-methyl-phenyl |
| 22A | cyclobutanyl | 1,4-phenylene | 3-methyl isooxazol-4,5-diyl | 5 | methyl | phenyl |
| 23A | oxetan-3-yl | 1,4-phenylene | 1,2,3-triazol-4,5-diyl | 5 | H | 2-chloro-phenyl |
| 24A | oxetan-3-yl | 1,4-phenylene | 1H-pyrazol-4,5-diyl | 4 | H | phenyl |
| 25A | oxetan-3-yl | 1,4-phenylene | 1H-pyrazol-4,5-diyl | 4 | H | 2-methyl-phenyl |
| 26A | cyclohexanyl | 1,4-phenylene | 3-methyl isooxazol-4,5-diyl | 5 | methyl | phenyl |
| 27A | oxetan-3-yl | 1,4-phenylene | 1H-pyrazol-4,5-diyl | 4 | H | 2-chloro-phenyl |
| 28A | oxetan-3-yl | 1,4-phenylene | 3-methyl isooxazol-4,5-diyl | 5 | H | 2-methyl-phenyl |
| 29A | cyclopentanyl | 1,4-phenylene | 3-methyl isooxazol-4,5-diyl | 5 | methyl | phenyl |
| 30A | oxetan-3-yl | 1,4-phenylene | isothiazol-3,4-diyl | 3 | H | 2-methyl-phenyl |
| 31A | oxetan-3-yl | 1,4-phenylene | 1,2,3-triazol-4,5-diyl | 5 | H | phenyl |
| 32A | oxetan-3-yl | 1,4-phenylene | 1,2,3-triazol-1,5-diyl | 1 | H | phenyl |
| 33A | oxetan-3-yl | 1,4-phenylene | 1,2,3-triazol-1,5-diyl | 1 | H | 2-methyl-phenyl |
| 34A | oxetan-3-yl | 1,4-phenylene | isothiazol-4,5-diyl | 5 | H | phenyl |
| 35A | oxetan-3-yl | 1,4-phenylene | isothiazol-4,5-diyl | 5 | H | 2-methyl-phenyl |
| 36A | oxetan-3-yl | 1,4-phenylene | isothiazol-4,5-diyl | 5 | H | 2-chloro-phenyl |
| 37A | oxetan-3-yl | 1,4-phenylene | 1,2,3-triazol-1,5-diyl | 1 | H | 2-chloro-phenyl |
| 38A | cyclopentanyl | 1,4-phenylene | isooxazol-4,5-diyl | 5 | H | 2-chloro-phenyl |
| 39A | cyclopropanyl | 1,4-phenylene | isooxazol-3,4-diyl | 3 | H | phenyl |
| 40A | cyclobutanyl | 1,4-phenylene | 1,2,3-triazol-4,5-diyl | 5 | H | phenyl |
| 41A | cyclopentanyl | 1,4-phenylene | 3-methyl isooxazol-4,5-diyl | 5 | H | phenyl |
| 42A | cyclobutanyl | 1,4-phenylene | isooxazol-3,4-diyl | 3 | H | phenyl |
| 43A | cyclohexanyl | 1,4-phenylene | isooxazol-3,4-diyl | 3 | H | phenyl |
| 44A | cyclopentanyl | 1,4-phenylene | isooxazol-3,4-diyl | 3 | H | phenyl |
| 45A | cyclohexanyl | 1,4-phenylene | isooxazol-3,4-diyl | 3 | H | 2-methyl-phenyl |
| 46A | cyclopentanyl | 1,4-phenylene | isooxazol-3,4-diyl | 3 | H | 2-methyl-phenyl |
| 47A | cyclobutanyl | 1,4-phenylene | 1,2,3-triazol-4,5-diyl | 5 | H | 2-methyl-phenyl |
| 48A | cyclobutanyl | 1,4-phenylene | isooxazol-3,4-diyl | 3 | H | 2-methyl-phenyl |
| 49A | cyclopropanyl | 1,4-phenylene | isooxazol-3,4-diyl | 3 | H | 2-methyl-phenyl |
| 50A | cyclohexanyl | 1,4-phenylene | 1H-pyrazol-4,5-diyl | 4 | H | 2-methyl-phenyl |
| 51A | cyclopentanyl | 1,4-phenylene | 3-methyl isooxazol-4,5-diyl | 5 | H | 2-methyl-phenyl |
| 52A | cyclopropanyl | 1,4-phenylene | isothiazol-4,5-diyl | 5 | H | phenyl |
| 53A | cyclopropanyl | 1,4-phenylene | isothiazol-4,5-diyl | 5 | H | 2-methyl-phenyl |
| 54A | cyclopropanyl | 1,4-phenylene | isothiazol-4,5-diyl | 5 | H | 2-chloro-phenyl |
| 55A | cyclopropanyl | 1,4-phenylene | 1,2,3-triazol-4,5-diyl | 5 | H | phenyl |
| 56A | cyclopropanyl | 1,4-phenylene | 1,2,3-triazol-1,5-diyl | 1 | H | 2-methyl-phenyl |
| 57A | cyclopropanyl | 1,4-phenylene | 1,2,3-triazol-4,5-diyl | 5 | H | 2-methyl-phenyl |
| 58A | cyclopropanyl | 1,4-phenylene | 1,2,3-triazol-4,5-diyl | 5 | H | 2-chloro-phenyl |
| 59A | cyclopropanyl | 1,4-phenylene | 1H-pyrazol-4,5-diyl | 4 | H | phenyl |
| 60A | cyclopropanyl | 1,4-phenylene | 1H-pyrazol-4,5-diyl | 4 | H | 2-methyl-phenyl |
| 61A | cyclopropanyl | 1,4-phenylene | 1H-pyrazol-4,5-diyl | 4 | H | 2-chloro-phenyl |
| 62A | cyclopropanyl | 1,4-phenylene | 3-methyl isooxazol-4,5-diyl | 5 | H | 2-methyl-phenyl |
| 63A | cyclopentanyl | 1,4-phenylene | isooxazol-3,4-diyl | 3 | H | 2-chloro-phenyl |
| 64A | cyclopropanyl | 1,4-phenylene | 3-methyl isooxazol-4,5-diyl | 5 | H | 2-chloro-phenyl |
| 65A | cyclopropanyl | 1,4-phenylene | isothiazol-3,4-diyl | 3 | H | phenyl |
| 66A | cyclopropanyl | 1,4-phenylene | isothiazol-3,4-diyl | 3 | H | 2-methyl-phenyl |
| 67A | cyclopropanyl | 1,4-phenylene | isothiazol-3,4-diyl | 3 | H | 2-chloro-phenyl |
| 68A | cyclobutanyl | 1,4-phenylene | isooxazol-3,4-diyl | 3 | H | 2-chloro-phenyl |
| 69A | cyclopropanyl | 1,4-phenylene | 1,2,3-triazol-1,5-diyl | 1 | H | phenyl |
| 70A | cyclopropanyl | 1,4-phenylene | 1,2,3-triazol-1,5-diyl | 1 | H | 2-chloro-phenyl |
| 71A | cyclohexanyl | 1,4-phenylene | isooxazol-3,4-diyl | 3 | H | 2-chloro-phenyl |
| 72A | cyclohexanyl | 1,4-phenylene | isothiazol-3,4-diyl | 3 | H | phenyl |
| 73A | cyclopentanyl | 1,4-phenylene | isothiazol-3,4-diyl | 3 | H | phenyl |
| 74A | cyclobutanyl | 1,4-phenylene | isothiazol-3,4-diyl | 3 | H | phenyl |
| 75A | cyclohexanyl | 1,4-phenylene | isothiazol-3,4-diyl | 3 | H | 2-methyl-phenyl |
| 76A | cyclopentanyl | 1,4-phenylene | isothiazol-3,4-diyl | 3 | H | 2-methyl-phenyl |
| 77A | cyclobutanyl | 1,4-phenylene | isothiazol-3,4-diyl | 3 | H | 2-methyl-phenyl |
| 78A | cyclohexanyl | 1,4-phenylene | isothiazol-3,4-diyl | 3 | H | 2-chloro-phenyl |
| 79A | cyclopentanyl | 1,4-phenylene | isothiazol-3,4-diyl | 3 | H | 2-chloro-phenyl |
| 80A | cyclobutanyl | 1,4-phenylene | isothiazol-3,4-diyl | 3 | H | 2-chloro-phenyl |
| 81A | cyclobutanyl | 1,4-phenylene | isothiazol-4,5-diyl | 5 | H | phenyl |
| 82A | cyclohexanyl | 1,4-phenylene | isothiazol-4,5-diyl | 5 | H | phenyl |
| 83A | cyclopentanyl | 1,4-phenylene | isothiazol-4,5-diyl | 5 | H | phenyl |
| 84A | cyclobutanyl | 1,4-phenylene | isothiazol-4,5-diyl | 5 | H | 2-methyl-phenyl |
| 85A | cyclohexanyl | 1,4-phenylene | isothiazol-4,5-diyl | 5 | H | 2-methyl-phenyl |
| 86A | cyclopentanyl | 1,4-phenylene | isothiazol-4,5-diyl | 5 | H | 2-methyl-phenyl |
| 87A | cyclobutanyl | 1,4-phenylene | isothiazol-4,5-diyl | 5 | H | 2-chloro-phenyl |
| 88A | cyclohexanyl | 1,4-phenylene | isothiazol-4,5-diyl | 5 | H | 2-chloro-phenyl |
| 89A | cyclopentanyl | 1,4-phenylene | isothiazol-4,5-diyl | 5 | H | 2-chloro-phenyl |
| 90A | cyclohexanyl | 1,4-phenylene | 1,2,3-triazol-4,5-diyl | 5 | H | phenyl |
| 91A | cyclopentanyl | 1,4-phenylene | 1,2,3-triazol-4,5-diyl | 5 | H | phenyl |
| 92A | cyclohexanyl | 1,4-phenylene | 1,2,3-triazol-4,5-diyl | 5 | H | 2-methyl-phenyl |
| 93A | cyclopentanyl | 1,4-phenylene | 1,2,3-triazol-4,5-diyl | 5 | H | 2-methyl-phenyl |
| 94A | cyclobutanyl | 1,4-phenylene | 1,2,3-triazol-1,5-diyl | 1 | H | 2-methyl-phenyl |
| 95A | cyclobutanyl | 1,4-phenylene | 1H-pyrazol-4,5-diyl | 4 | H | 2-methyl-phenyl |
| 96A | cyclobutanyl | 1,4-phenylene | 3-methyl isooxazol-4,5-diyl | 5 | H | 2-methyl-phenyl |
| 97A | cyclobutanyl | 1,4-phenylene | 1H-pyrazol-4,5-diyl | 4 | H | phenyl |
| 98A | cyclopentanyl | 1,4-phenylene | 1,2,3-triazol-4,5-diyl | 5 | H | 2-chloro-phenyl |
| 99A | cyclobutanyl | 1,4-phenylene | 1,2,3-triazol-1,5-diyl | 1 | H | phenyl |
| 100A | cyclobutanyl | 1,4-phenylene | 1,2,3-triazol-1,5-diyl | 1 | H | 2-chloro-phenyl |
| 101A | cyclohexanyl | 1,4-phenylene | 1,2,3-triazol-4,5-diyl | 5 | H | 2-chloro-phenyl |
| 102A | cyclobutanyl | 1,4-phenylene | 1H-pyrazol-4,5-diyl | 4 | H | 2-chloro-phenyl |
| 103A | cyclobutanyl | 1,4-phenylene | 3-methyl isooxazol-4,5-diyl | 5 | H | 2-chloro-phenyl |
| 104A | cyclohexanyl | 1,4-phenylene | 1,2,3-triazol-1,5-diyl | 1 | H | phenyl |
| 105A | cyclopentanyl | 1,4-phenylene | 1,2,3-triazol-1,5-diyl | 1 | H | phenyl |
| 106A | cyclohexanyl | 1,4-phenylene | 1,2,3-triazol-1,5-diyl | 1 | H | 2-methyl-phenyl |
| 107A | cyclopentanyl | 1,4-phenylene | 1,2,3-triazol-1,5-diyl | 1 | H | 2-methyl-phenyl |
| 108A | cyclohexanyl | 1,4-phenylene | 1,2,3-triazol-1,5-diyl | 1 | H | 2-chloro-phenyl |
| 109A | cyclopentanyl | 1,4-phenylene | 1,2,3-triazol-1,5-diyl | 1 | H | 2-chloro-phenyl |
| 110A | cyclopentanyl | 1,4-phenylene | 1H-pyrazol-4,5-diyl | 4 | H | phenyl |
| 111A | cyclohexanyl | 1,4-phenylene | 1H-pyrazol-4,5-diyl | 4 | H | phenyl |
| 112A | cyclopentanyl | 1,4-phenylene | 1H-pyrazol-4,5-diyl | 4 | H | 2-methyl-phenyl |
| 113A | cyclohexanyl | 1,4-phenylene | 3-methyl isooxazol-4,5-diyl | 5 | H | 2-methyl-phenyl |
| 114A | cyclopentanyl | 1,4-phenylene | 1H-pyrazol-4,5-diyl | 4 | H | 2-chloro-phenyl |
| 115A | cyclohexanyl | 1,4-phenylene | 3-methyl isooxazol-4,5-diyl | 5 | H | 2-chloro-phenyl |
| 116A | oxetan-3-yl | pyridin-2-one-1,4-diyl | 1,2,3-triazol-4,5-diyl | 5 | H | 2-methyl-phenyl |
| 117A | cyclopropanyl | pyridin-2-one-1,4-diyl | 1,2,3-triazol-4,5-diyl | 5 | H | 2-methyl-phenyl |
| 18A | thiazol-4-yl | pyridin-2-one-1,4-diyl | 1,2,3-triazol-4,5-diyl | 5 | H | 2-methyl-phenyl |
| 119A | cyclopropanyl | pyridin-2-one-1,4-diyl | 1,2,3-triazol-4,5-diyl | 5 | H | 2-methyl-phenyl |
| 120A | oxazol-4-yl | pyridin-2-one-1,4-diyl | 1,2,3-triazol-4,5-diyl | 5 | H | 2-methyl-phenyl |
| 121A | cyclohexanyl | pyridin-2-one-1,4-diyl | 1,2,3-triazol-4,5-diyl | 5 | H | 2-methyl-phenyl |
| 122A | cyclopentanyl | pyridin-2-one-1,4-diyl | 1,2,3-triazol-4,5-diyl | 5 | H | 2-methyl-phenyl |

Note:
column * indicates ring C's point of connection to the adjacent optionally substituted phen-1,4-ylene.

In some embodiments, compounds of Formula (XII) are also represented by Formula (XII-B) and are selected from the following compounds as listed in Table 12B.

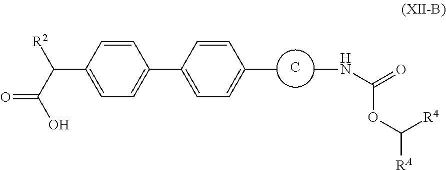

TABLE 12B

| Cmpd # XII- | R² | 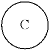 | * | R⁴ | R^{A} |
|---|---|---|---|---|---|
| 1B | thiazol-2-yl | isothiazol-3,4-diyl | 3 | methyl | phenyl |
| 2B | thiazol-4-yl | isothiazol-3,4-diyl | 3 | methyl | phenyl |
| 3B | oxazol-4-yl | 1,2,3-triazol-1,5-diyl | 1 | methyl | phenyl |
| 4B | thiazol-2-yl | isothiazol-3,4-diyl | 3 | methyl | 2-methyl-phenyl |
| 5B | thiazol-4-yl | isothiazol-3,4-diyl | 3 | methyl | 2-methyl-phenyl |
| 6B | oxazol-4-yl | 1,2,3-triazol-1,5-diyl | 1 | methyl | 2-chloro-phenyl |
| 7B | thiazol-2-yl | 5-methyl isothiazol- | 3 | H | 2-methyl-phenyl |
| | | 3,4-diyl | | | |
| 8B | thiazol-4-yl | 5-methyl isothiazol- | 3 | H | 2-methyl-phenyl |
| | | 3,4-diyl | | | |
| 9B | oxazol-4-yl | 4-methyl 1,2,3-triazol- | 1 | H | phenyl |
| | | 1,5-diyl | | | |
| 10B | oxazol-4-yl | 4-methyl 1,2,3-triazol- | 1 | H | 2-chloro-phenyl |
| | | 1,5-diyl | | | |
| 11B | thiazol-2-yl | 3-methyl isooxazol- | 5 | methyl | phenyl |
| | | 4,5-diyl | | | |
| 12B | thiazol-4-yl | 3-methyl isooxazol- | 5 | methyl | phenyl |
| | | 4,5-diyl | | | |
| 13B | oxazol-2-yl | 3-methyl isooxazol- | 5 | methyl | phenyl |
| | | 4,5-diyl | | | |
| 14B | oxazol-4-yl | 3-methyl isooxazol- | 5 | methyl | phenyl |
| | | 4,5-diyl | | | |
| 15B | thiazol-2-yl | isooxazol-3,4-diyl | 3 | H | phenyl |
| 16B | thiazol-4-yl | isooxazol-3,4-diyl | 3 | H | phenyl |
| 17B | oxazol-2-yl | isooxazol-3,4-diyl | 3 | H | phenyl |
| 18B | oxazol-4-yl | isooxazol-3,4-diyl | 3 | H | phenyl |
| 19B | thiazol-2-yl | isooxazol-3,4-diyl | 3 | H | 2-methyl-phenyl |
| 20B | thiazol-4-yl | isooxazol-3,4-diyl | 3 | H | 2-methyl-phenyl |
| 21B | oxazol-2-yl | isooxazol-3,4-diyl | 3 | H | 2-methyl-phenyl |
| 22B | oxazol-4-yl | isooxazol-3,4-diyl | 3 | H | 2-methyl-phenyl |
| 23B | oxazol-4-yl | 1,2,3-triazol-1,5-diyl | 1 | H | 2-methyl-phenyl |
| 24B | thiazol-2-yl | isooxazol-3,4-diyl | 3 | H | 2-chloro-phenyl |
| 25B | thiazol-4-yl | isooxazol-3,4-diyl | 3 | H | 2-chloro-phenyl |
| 26B | oxazol-2-yl | isooxazol-3,4-diyl | 3 | H | 2-chloro-phenyl |
| 27B | oxazol-4-yl | isooxazol-3,4-diyl | 3 | H | 2-chloro-phenyl |
| 28B | thiazol-2-yl | isothiazol-3,4-diyl | 3 | H | 2-chloro-phenyl |
| 29B | thiazol-4-yl | isothiazol-3,4-diyl | 3 | H | 2-chloro-phenyl |
| 30B | oxazol-2-yl | isothiazol-3,4-diyl | 3 | H | phenyl |
| 31B | oxazol-4-yl | isothiazol-3,4-diyl | 3 | H | phenyl |
| 32B | thiazol-2-yl | isothiazol-3,4-diyl | 3 | H | phenyl |
| 33B | thiazol-4-yl | isothiazol-3,4-diyl | 3 | H | phenyl |
| 34B | thiazol-2-yl | isothiazol-4,5-diyl | 5 | H | phenyl |
| 35B | thiazol-4-yl | isothiazol-4,5-diyl | 5 | H | phenyl |
| 36B | thiazol-2-yl | isothiazol-4,5-diyl | 5 | H | 2-methyl-phenyl |
| 37B | thiazol-4-yl | isothiazol-4,5-diyl | 5 | H | 2-methyl-phenyl |
| 38B | thiazol-2-yl | isothiazol-4,5-diyl | 5 | H | 2-chloro-phenyl |
| 39B | thiazol-4-yl | isothiazol-4,5-diyl | 5 | H | 2-chloro-phenyl |
| 40B | thiazol-4-yl | 1,2,3-triazol-4,5-diyl | 5 | H | 2-methyl-phenyl |
| 41B | oxazol-2-yl | isothiazol-3,4-diyl | 3 | H | 2-methyl-phenyl |
| 42B | oxazol-4-yl | isothiazol-3,4-diyl | 3 | H | 2-methyl-phenyl |
| 43B | thiazol-4-yl | 1,2,3-triazol-1,5-diyl | 1 | H | 2-chloro-phenyl |
| 44B | thiazol-4-yl | 1H-pyrazol-4,5-diyl | 4 | H | phenyl |
| 45B | thiazol-4-yl | 1H-pyrazol-4,5-diyl | 4 | H | 2-methyl-phenyl |
| 46B | thiazol-4-yl | 1,2,3-triazol-4,5-diyl | 5 | H | phenyl |
| 47B | thiazol-4-yl | 1,2,3-triazol-1,5-diyl | 1 | H | phenyl |
| 48B | thiazol-4-yl | 1,2,3-triazol-1,5-diyl | 1 | H | 2-methyl-phenyl |
| 49B | thiazol-4-yl | 1H-pyrazol-4,5-diyl | 4 | H | 2-chloro-phenyl |
| 50B | thiazol-2-yl | 3-methyl isooxazol- | 5 | H | 2-methyl-phenyl |
| | | 4,5-diyl | | | |
| 51B | thiazol-4-yl | 3-methyl isooxazol- | 5 | H | 2-methyl-phenyl |
| | | 4,5-diyl | | | |
| 52B | thiazol-4-yl | 1,2,3-triazol-4,5-diyl | 5 | H | 2-chloro-phenyl |
| 53B | thiazol-2-yl | 3-methyl isooxazol- | 5 | H | 2-chloro-phenyl |
| | | 4,5-diyl | | | |
| 54B | thiazol-4-yl | 3-methyl isooxazol- | 5 | H | 2-chloro-phenyl |
| | | 4,5-diyl | | | |
| 55B | oxazol-2-yl | isothiazol-3,4-diyl | 3 | H | 2-chloro-phenyl |
| 56B | oxazol-4-yl | isothiazol-3,4-diyl | 3 | H | 2-chloro-phenyl |
| 57B | oxazol-2-yl | isothiazol-4,5-diyl | 5 | H | phenyl |
| 58B | oxazol-4-yl | isothiazol-4,5-diyl | 5 | H | phenyl |
| 59B | oxazol-2-yl | isothiazol-4,5-diyl | 5 | H | 2-methyl-phenyl |
| 60B | oxazol-4-yl | isothiazol-4,5-diyl | 5 | H | 2-methyl-phenyl |
| 61B | oxazol-2-yl | isothiazol-4,5-diyl | 5 | H | 2-chloro-phenyl |
| 62B | oxazol-4-yl | isothiazol-4,5-diyl | 5 | H | 2-chloro-phenyl |
| 63B | oxazol-4-yl | 1,2,3-triazol-4,5-diyl | 5 | H | phenyl |
| 64B | oxazol-4-yl | 1,2,3-triazol-4,5-diyl | 5 | H | 2-methyl-phenyl |
| 65B | oxazol-4-yl | 1,2,3-triazol-4,5-diyl | 5 | H | 2-chloro-phenyl |
| 66B | oxazol-4-yl | 1H-pyrazol-4,5-diyl | 4 | H | phenyl |
| 67B | oxazol-4-yl | 1H-pyrazol-4,5-diyl | 4 | H | 2-methyl-phenyl |
| 68B | oxazol-2-yl | 3-methyl isooxazol- | 5 | H | 2-methyl-phenyl |
| | | 4,5-diyl | | | |
| 69B | oxazol-4-yl | 3-methyl isooxazol- | 5 | H | 2-methyl-phenyl |
| | | 4,5-diyl | | | |
| 70B | oxazol-2-yl | 3-methyl isooxazol- | 5 | H | 2-chloro-phenyl |
| | | 4,5-diyl | | | |
| 71B | oxazol-4-yl | 3-methyl isooxazol- | 5 | H | 2-chloro-phenyl |
| | | 4,5-diyl | | | |
| 72B | oxazol-4-yl | 1,2,3-triazol-1,5-diyl | 1 | H | phenyl |
| 73B | oxazol-4-yl | 1,2,3-triazol-1,5-diyl | 1 | H | 2-chloro-phenyl |
| 74B | oxazol-4-yl | 1H-pyrazol-4,5-diyl | 4 | H | 2-chloro-phenyl |

Note:
column * indicates ring C's point of connection to the adjacent phen-1,4-ylene.

Some embodiments of compounds described herein are selected from the following compounds as listed in Table 13.

TABLE 13

| Compd. | Structure |
|---|---|
| IT001 | 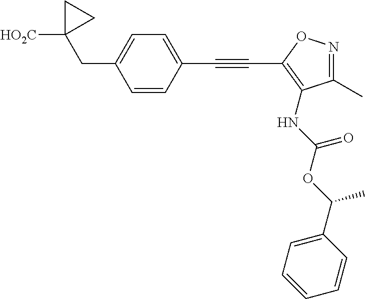 [SMILES: Cc1noc(C#Cc2ccc(CC3(C(=O)O)CC3)cc2)c1NC(=O)O[C@H](C)c1ccccc1] |
| IT002 | 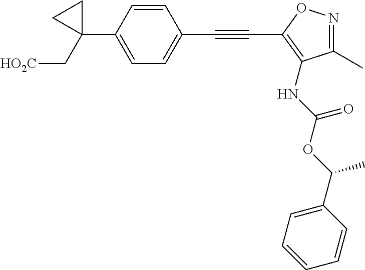 [SMILES: Cc1noc(C#Cc2ccc(C3(CC(=O)O)CC3)cc2)c1NC(=O)O[C@H](C)c1ccccc1] |
| IT003 | 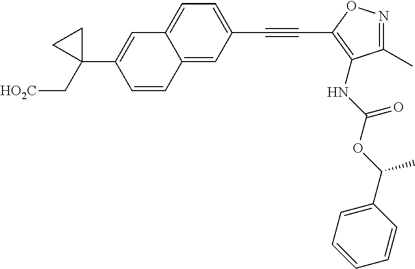 [SMILES: Cc1noc(C#Cc2ccc3cc(C4(CC(=O)O)CC4)ccc3c2)c1NC(=O)O[C@H](C)c1ccccc1] |
| IT004 | 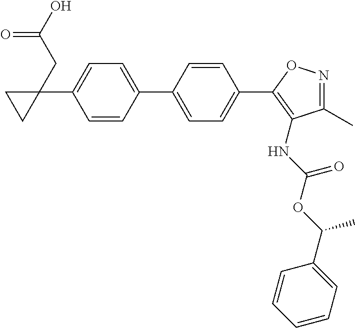 [SMILES: Cc1noc(-c2ccc(-c3ccc(C4(CC(=O)O)CC4)cc3)cc2)c1NC(=O)O[C@H](C)c1ccccc1] |
| IT005 | 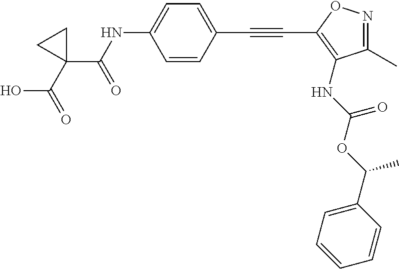 [SMILES: Cc1noc(C#Cc2ccc(NC(=O)C3(C(=O)O)CC3)cc2)c1NC(=O)O[C@H](C)c1ccccc1] |
| IT006 | 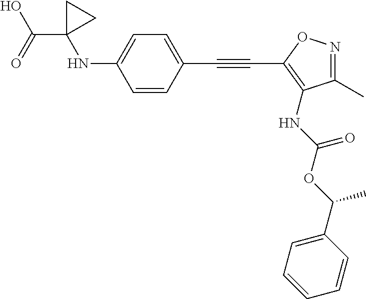 [SMILES: Cc1noc(C#Cc2ccc(NC3(C(=O)O)CC3)cc2)c1NC(=O)O[C@H](C)c1ccccc1] |
| IT007 | 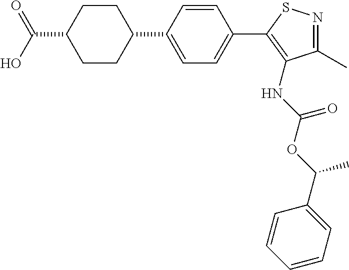 [SMILES: Cc1nsc(-c2ccc([C@H]3CC[C@@H](C(=O)O)CC3)cc2)c1NC(=O)O[C@H](C)c1ccccc1] |
| | Isomer 1 |
| IT008 | 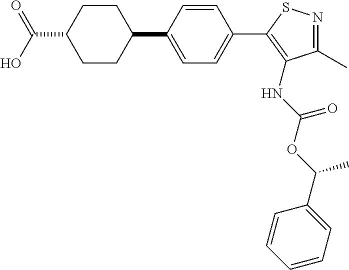 [SMILES: Cc1nsc(-c2ccc([C@H]3CC[C@H](C(=O)O)CC3)cc2)c1NC(=O)O[C@H](C)c1ccccc1] |
| | Isomer 2 |
| IT009 | 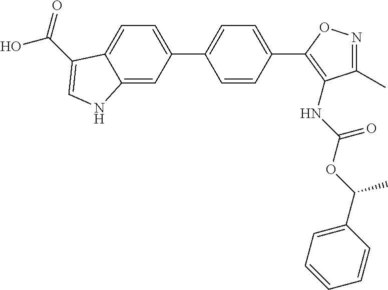 [SMILES: Cc1noc(-c2ccc(-c3ccc4c(C(=O)O)c[nH]c4c3)cc2)c1NC(=O)O[C@H](C)c1ccccc1] |
| IT010 | 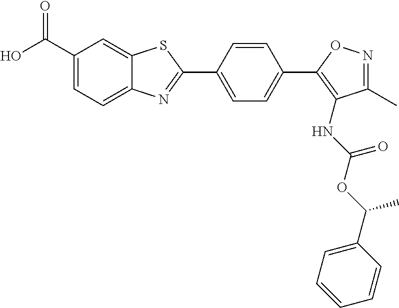 [SMILES: Cc1noc(-c2ccc(-c3nc4ccc(C(=O)O)cc4s3)cc2)c1NC(=O)O[C@H](C)c1ccccc1] |
| IT011 | 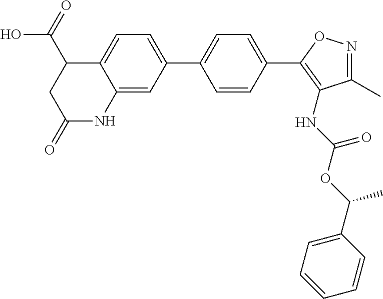 [SMILES: Cc1noc(-c2ccc(-c3ccc4c(c3)NC(=O)CC4C(=O)O)cc2)c1NC(=O)O[C@H](C)c1ccccc1] |
| IT012 | 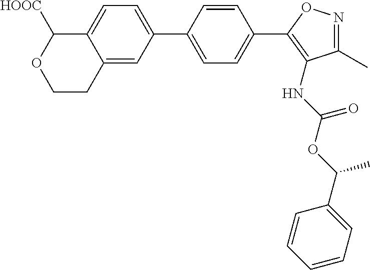 [SMILES: Cc1noc(-c2ccc(-c3ccc4c(c3)CCOC4C(=O)O)cc2)c1NC(=O)O[C@H](C)c1ccccc1] |
| IT013 | 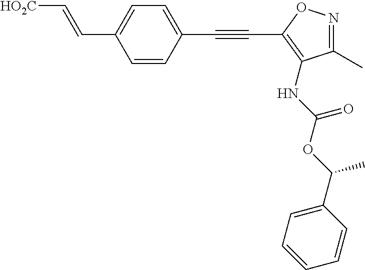 [SMILES: Cc1noc(C#Cc2ccc(/C=C/C(=O)O)cc2)c1NC(=O)O[C@H](C)c1ccccc1] |
| IT014 | 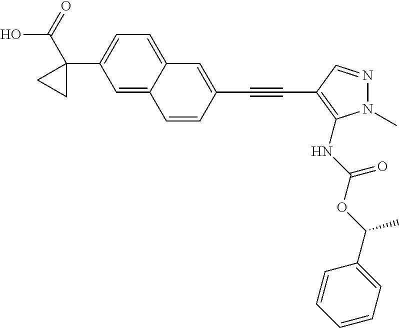 [SMILES: C[C@@H](OC(=O)Nc1c(C#Cc2ccc3cc(C4(C(=O)O)CC4)ccc3c2)cnn1C)c1ccccc1] |
| IT015 | 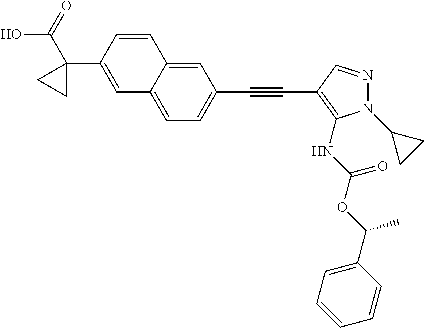 [SMILES: C[C@@H](OC(=O)Nc1c(C#Cc2ccc3cc(C4(C(=O)O)CC4)ccc3c2)cnn1C1CC1)c1ccccc1] |
| IT016 | 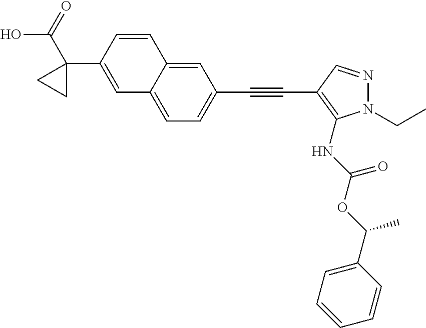 [SMILES: CCn1ncc(C#Cc2ccc3cc(C4(C(=O)O)CC4)ccc3c2)c1NC(=O)O[C@H](C)c1ccccc1] |
| IT017 | 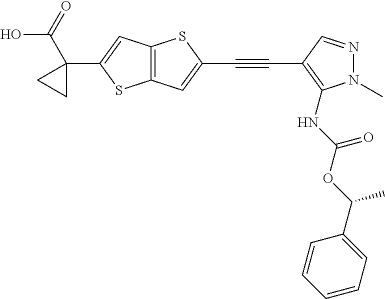 [SMILES: C[C@@H](OC(=O)Nc1c(C#Cc2cc3sc(C4(C(=O)O)CC4)cc3s2)cnn1C)c1ccccc1] |
| IT018 | 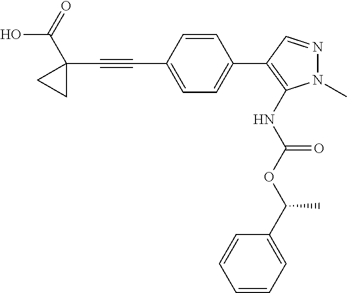 [SMILES: C[C@@H](OC(=O)Nc1c(-c2ccc(C#CC3(C(=O)O)CC3)cc2)cnn1C)c1ccccc1] |
| IT019 | 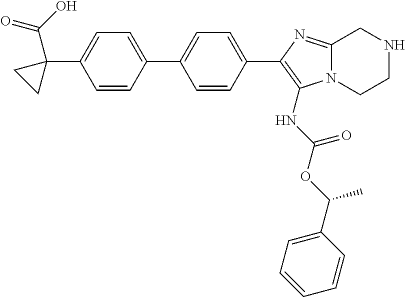 [SMILES: C[C@@H](OC(=O)Nc1c(-c2ccc(-c3ccc(C4(C(=O)O)CC4)cc3)cc2)nc2n1CCNC2)c1ccccc1] |
| IT020 | 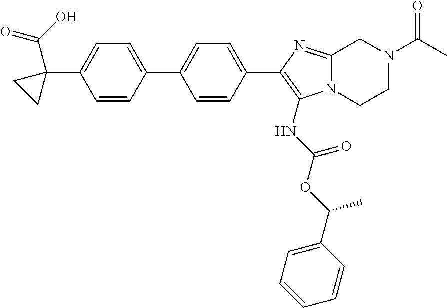 [SMILES: CC(=O)N1CCn2c(nc(-c3ccc(-c4ccc(C5(C(=O)O)CC5)cc4)cc3)c2NC(=O)O[C@H](C)c2ccccc2)C1] |
| IT021 | 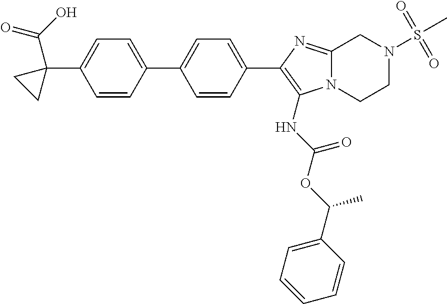 [SMILES: C[C@@H](OC(=O)Nc1c(-c2ccc(-c3ccc(C4(C(=O)O)CC4)cc3)cc2)nc2n1CCN(S(C)(=O)=O)C2)c1ccccc1] |
| IT022 | 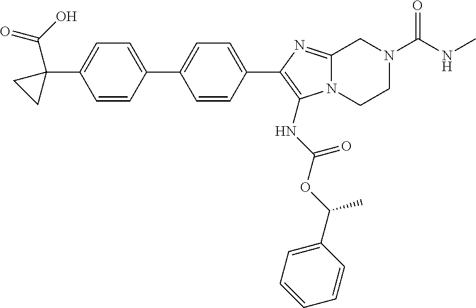 [SMILES: CNC(=O)N1CCn2c(nc(-c3ccc(-c4ccc(C5(C(=O)O)CC5)cc4)cc3)c2NC(=O)O[C@H](C)c2ccccc2)C1] |
| IT023 | 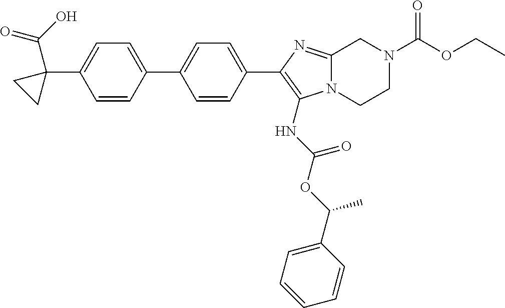 [SMILES: CCOC(=O)N1CCn2c(nc(-c3ccc(-c4ccc(C5(C(=O)O)CC5)cc4)cc3)c2NC(=O)O[C@H](C)c2ccccc2)C1] |
| IT024 | 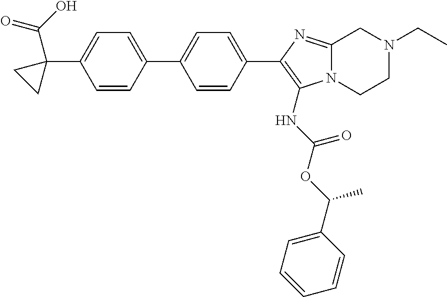 [SMILES: CCN1CCn2c(nc(-c3ccc(-c4ccc(C5(C(=O)O)CC5)cc4)cc3)c2NC(=O)O[C@H](C)c2ccccc2)C1] |
| IT025 | 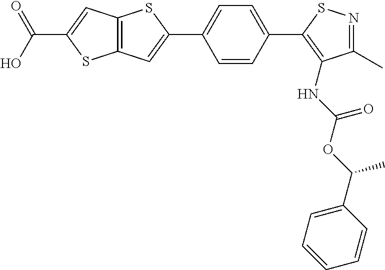 [SMILES: Cc1nsc(-c2ccc(-c3cc4sc(C(=O)O)cc4s3)cc2)c1NC(=O)O[C@H](C)c1ccccc1] |
| IT026 | 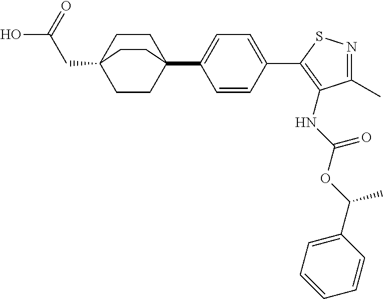 |
| IT027 | 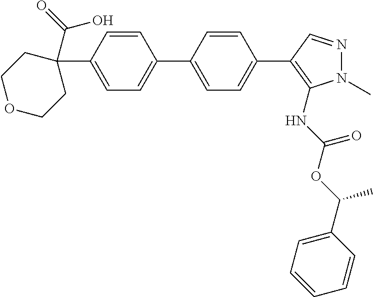 [SMILES: C[C@@H](OC(=O)Nc1c(-c2ccc(-c3ccc(C4(C(=O)O)CCOCC4)cc3)cc2)cnn1C)c1ccccc1] |
| IT028 | 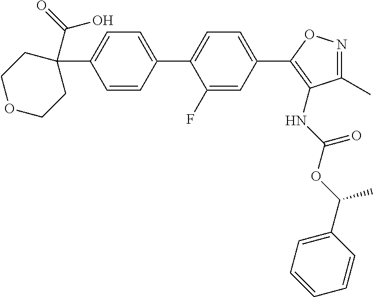 [SMILES: Cc1noc(-c2ccc(-c3ccc(C4(C(=O)O)CCOCC4)cc3)c(F)c2)c1NC(=O)O[C@H](C)c1ccccc1] |
| IT029 | 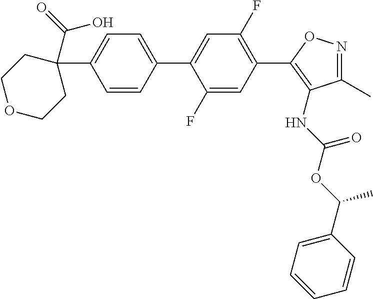 [SMILES: Cc1noc(-c2cc(F)c(-c3ccc(C4(C(=O)O)CCOCC4)cc3)cc2F)c1NC(=O)O[C@H](C)c1ccccc1] |
| IT030 | 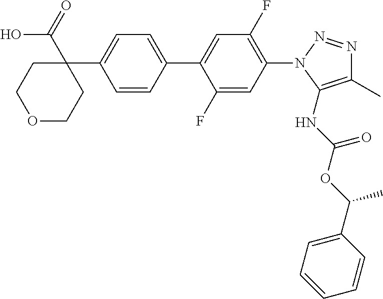 [SMILES: Cc1nnn(-c2cc(F)c(-c3ccc(C4(C(=O)O)CCOCC4)cc3)cc2F)c1NC(=O)O[C@H](C)c1ccccc1] |
| IT031 | 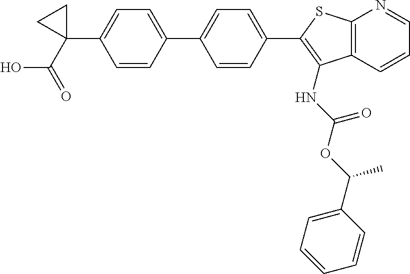 [SMILES: C[C@@H](OC(=O)Nc1c(-c2ccc(-c3ccc(C4(C(=O)O)CC4)cc3)cc2)sc2ncccc12)c1ccccc1] |
| IT032 | 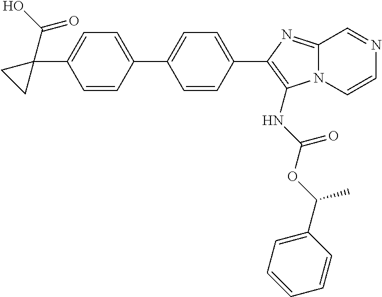 [SMILES: C[C@@H](OC(=O)Nc1c(-c2ccc(-c3ccc(C4(C(=O)O)CC4)cc3)cc2)nc2cnccn12)c1ccccc1] |
| IT033 | 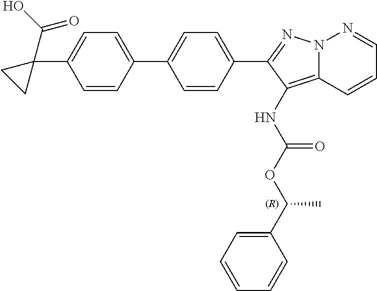 |
| IT034 | 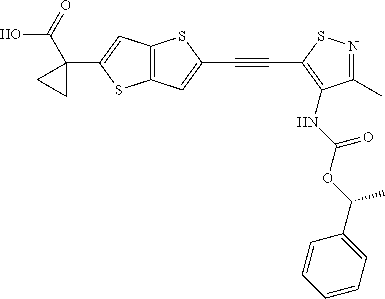 [SMILES: Cc1nsc(C#Cc2cc3sc(C4(C(=O)O)CC4)cc3s2)c1NC(=O)O[C@H](C)c1ccccc1] |
| IT035 | 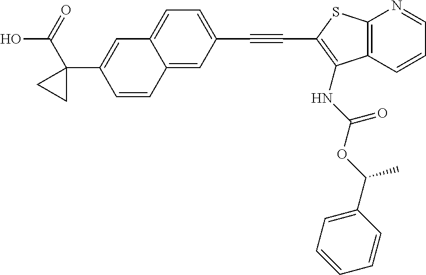 [SMILES: C[C@@H](OC(=O)Nc1c(C#Cc2ccc3cc(C4(C(=O)O)CC4)ccc3c2)sc2ncccc12)c1ccccc1] |
| IT036 | 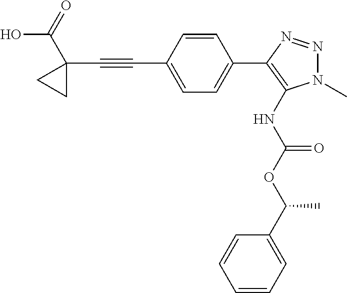 [SMILES: C[C@@H](OC(=O)Nc1c(-c2ccc(C#CC3(C(=O)O)CC3)cc2)nnn1C)c1ccccc1] |
| IT037 | 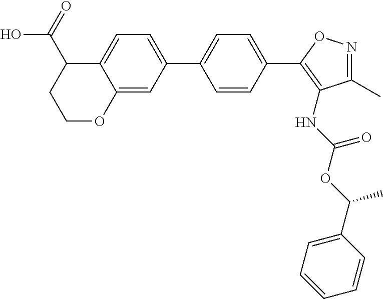 [SMILES: Cc1noc(-c2ccc(-c3ccc4c(c3)OCCC4C(=O)O)cc2)c1NC(=O)O[C@H](C)c1ccccc1] |
| IT038 | 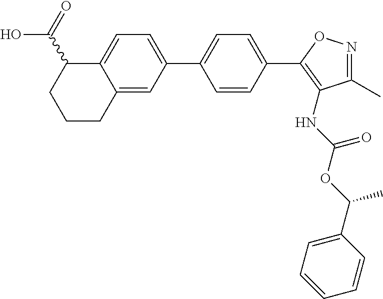 [SMILES: Cc1noc(-c2ccc(-c3ccc4c(c3)CCCC4C(=O)O)cc2)c1NC(=O)O[C@H](C)c1ccccc1] |
| | Isomer 1 |
| IT039 | 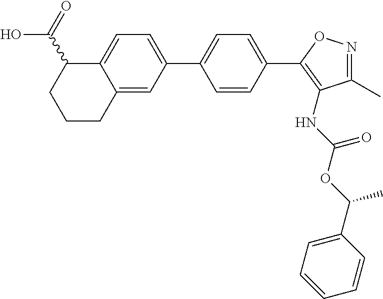 [SMILES: Cc1noc(-c2ccc(-c3ccc4c(c3)CCCC4C(=O)O)cc2)c1NC(=O)O[C@H](C)c1ccccc1] |
| | Isomer 2 |
| IT040 | 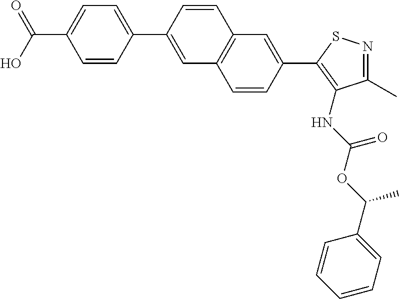 [SMILES: Cc1nsc(-c2ccc3cc(-c4ccc(C(=O)O)cc4)ccc3c2)c1NC(=O)O[C@H](C)c1ccccc1] |
| IT041 | 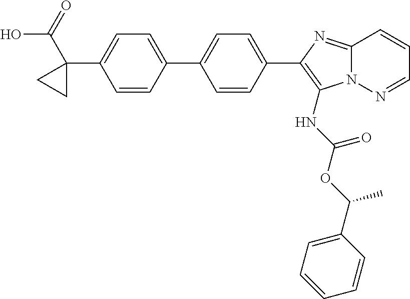 [SMILES: C[C@@H](OC(=O)Nc1c(-c2ccc(-c3ccc(C4(C(=O)O)CC4)cc3)cc2)nc2cccnn12)c1ccccc1] |
| IT042 | 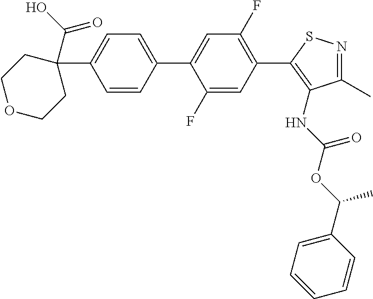 [SMILES: Cc1nsc(-c2cc(F)c(-c3ccc(C4(C(=O)O)CCOCC4)cc3)cc2F)c1NC(=O)O[C@H](C)c1ccccc1] |
| IT043 | 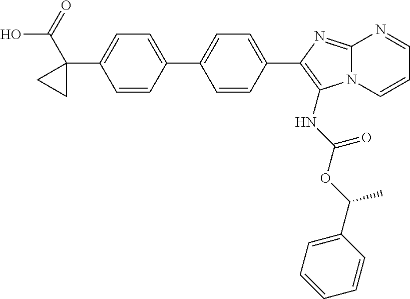 [SMILES: C[C@@H](OC(=O)Nc1c(-c2ccc(-c3ccc(C4(C(=O)O)CC4)cc3)cc2)nc2ncccn12)c1ccccc1] |
| IT044 | 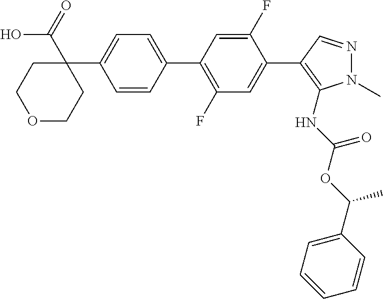 [SMILES: C[C@@H](OC(=O)Nc1c(-c2cc(F)c(-c3ccc(C4(C(=O)O)CCOCC4)cc3)cc2F)cnn1C)c1ccccc1] |
| IT045 | 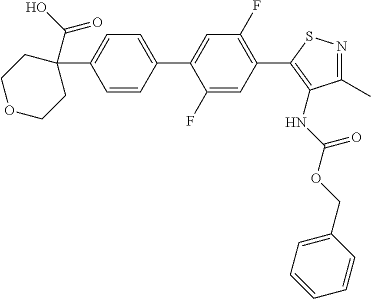 [SMILES: Cc1nsc(-c2cc(F)c(-c3ccc(C4(C(=O)O)CCOCC4)cc3)cc2F)c1NC(=O)OCc1ccccc1] |
| IT046 | 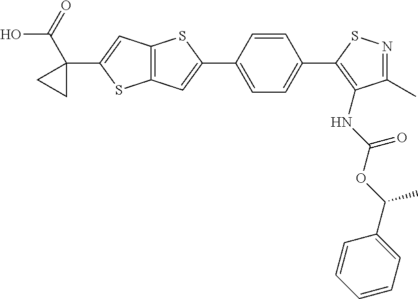 [SMILES: Cc1nsc(-c2ccc(-c3cc4sc(C5(C(=O)O)CC5)cc4s3)cc2)c1NC(=O)O[C@H](C)c1ccccc1] |
| IT047 | 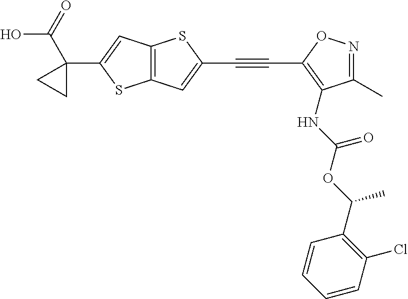 [SMILES: Cc1noc(C#Cc2cc3sc(C4(C(=O)O)CC4)cc3s2)c1NC(=O)O[C@H](C)c1ccccc1Cl] |
| IT048 | 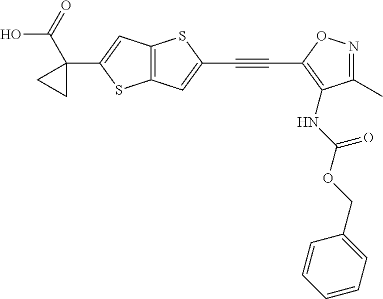 [SMILES: Cc1noc(C#Cc2cc3sc(C4(C(=O)O)CC4)cc3s2)c1NC(=O)OCc1ccccc1] |
| IT049 | 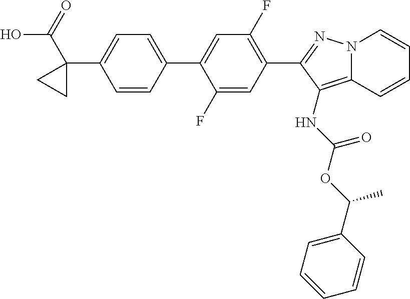 [SMILES: C[C@@H](OC(=O)Nc1c(-c2cc(F)c(-c3ccc(C4(C(=O)O)CC4)cc3)cc2F)nn2ccccc12)c1ccccc1] |
| IT050 | 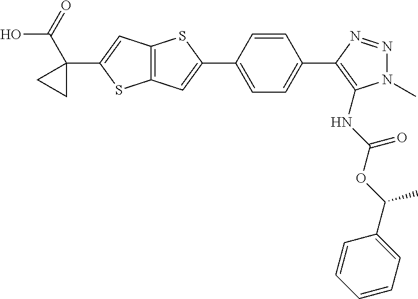 [SMILES: C[C@@H](OC(=O)Nc1c(-c2ccc(-c3cc4sc(C5(C(=O)O)CC5)cc4s3)cc2)nnn1C)c1ccccc1] |
| IT051 | 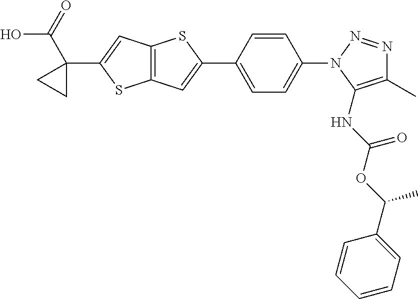 [SMILES: Cc1nnn(-c2ccc(-c3cc4sc(C5(C(=O)O)CC5)cc4s3)cc2)c1NC(=O)O[C@H](C)c1ccccc1] |
| IT052 | 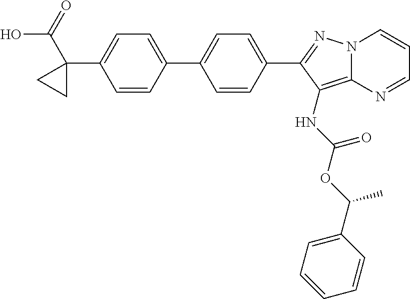 [SMILES: C[C@@H](OC(=O)Nc1c(-c2ccc(-c3ccc(C4(C(=O)O)CC4)cc3)cc2)nn2cccnc12)c1ccccc1] |
| IT053 | 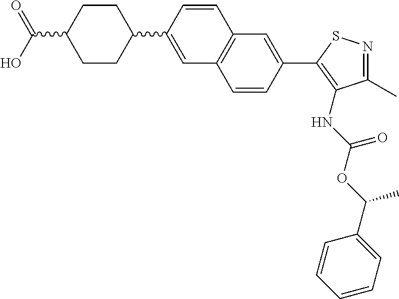 [SMILES: Cc1nsc(-c2ccc3cc(C4CCC(C(=O)O)CC4)ccc3c2)c1NC(=O)O[C@H](C)c1ccccc1] |
| IT054 | 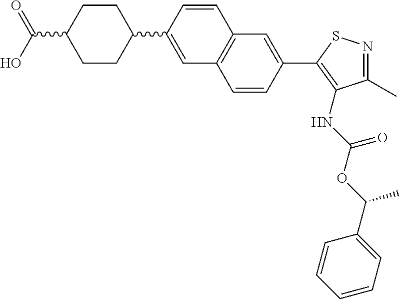 [SMILES: Cc1nsc(-c2ccc3cc(C4CCC(C(=O)O)CC4)ccc3c2)c1NC(=O)O[C@H](C)c1ccccc1] |
| IT055 | 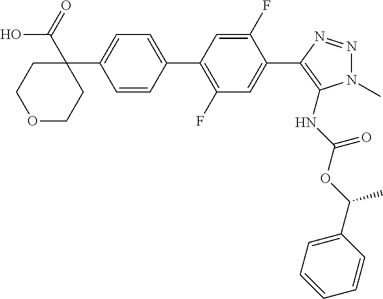 [SMILES: C[C@@H](OC(=O)Nc1c(-c2cc(F)c(-c3ccc(C4(C(=O)O)CCOCC4)cc3)cc2F)nnn1C)c1ccccc1] |
| IT056 | 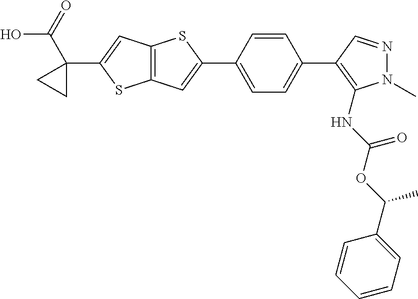 [SMILES: C[C@@H](OC(=O)Nc1c(-c2ccc(-c3cc4sc(C5(C(=O)O)CC5)cc4s3)cc2)cnn1C)c1ccccc1] |
| IT057 | 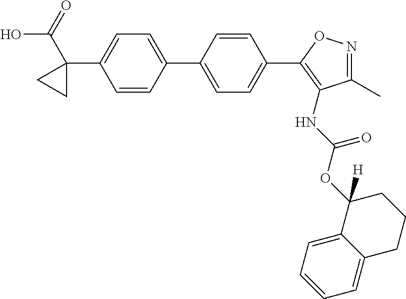 [SMILES: Cc1noc(-c2ccc(-c3ccc(C4(C(=O)O)CC4)cc3)cc2)c1NC(=O)O[C@@H]1CCCc2ccccc21] |
| | Isomer 1 |
| IT058 | 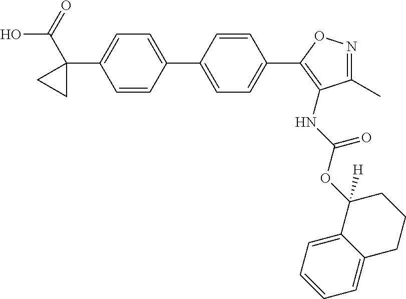 [SMILES: Cc1noc(-c2ccc(-c3ccc(C4(C(=O)O)CC4)cc3)cc2)c1NC(=O)O[C@H]1CCCc2ccccc21] |
| | Isomer 2 |
| IT059 | 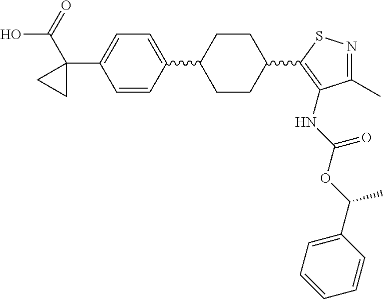 [SMILES: Cc1nsc(C2CCC(c3ccc(C4(C(=O)O)CC4)cc3)CC2)c1NC(=O)O[C@H](C)c1ccccc1] |
| | Isomer 1 |
| IT060 | 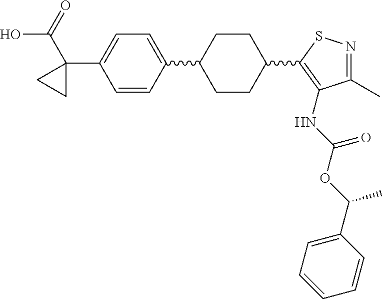 [SMILES: Cc1nsc(C2CCC(c3ccc(C4(C(=O)O)CC4)cc3)CC2)c1NC(=O)O[C@H](C)c1ccccc1] |
| | Isomer 2 |
| IT061 | 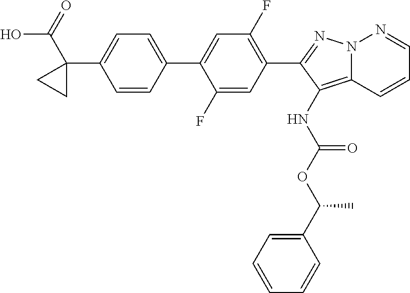 [SMILES: C[C@@H](OC(=O)Nc1c(-c2cc(F)c(-c3ccc(C4(C(=O)O)CC4)cc3)cc2F)nn2ncccc12)c1ccccc1] |
| IT062 | 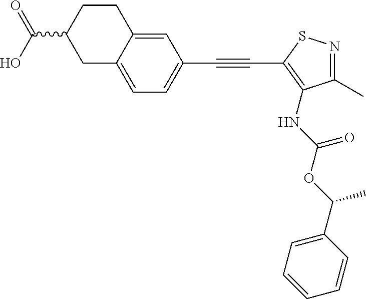 [SMILES: Cc1nsc(C#Cc2ccc3c(c2)CCC(C(=O)O)C3)c1NC(=O)O[C@H](C)c1ccccc1] |
| | Isomer 1 |
| IT063 | 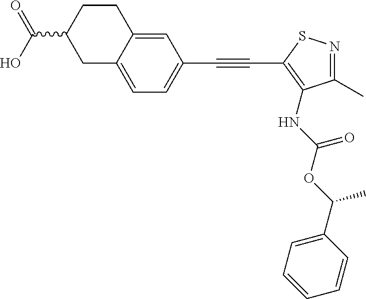 [SMILES: Cc1nsc(C#Cc2ccc3c(c2)CCC(C(=O)O)C3)c1NC(=O)O[C@H](C)c1ccccc1] |
| | Isomer 2 |
| IT064 | 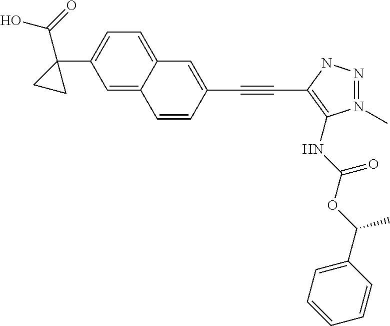 |
| IT065 | 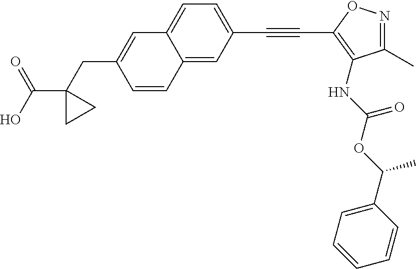 [SMILES: Cc1noc(C#Cc2ccc3cc(CC4(C(=O)O)CC4)ccc3c2)c1NC(=O)O[C@H](C)c1ccccc1] |
| IT066 | 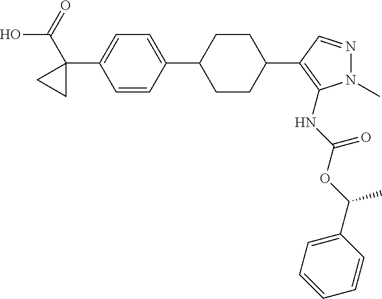 [SMILES: C[C@@H](OC(=O)Nc1c(C2CCC(c3ccc(C4(C(=O)O)CC4)cc3)CC2)cnn1C)c1ccccc1] |
| IT067 | 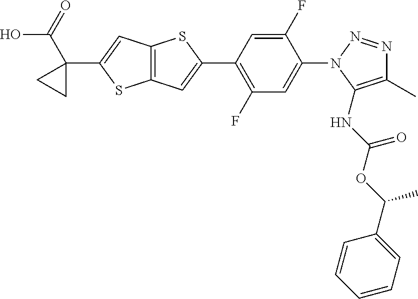 [SMILES: Cc1nnn(-c2cc(F)c(-c3cc4sc(C5(C(=O)O)CC5)cc4s3)cc2F)c1NC(=O)O[C@H](C)c1ccccc1] |
| IT068 | 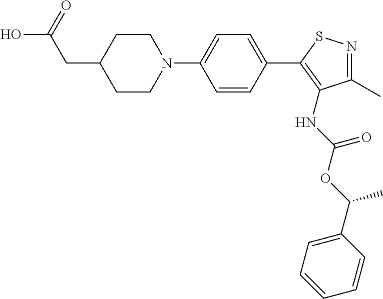 [SMILES: Cc1nsc(-c2ccc(N3CCC(CC(=O)O)CC3)cc2)c1NC(=O)O[C@H](C)c1ccccc1] |
| IT069 | 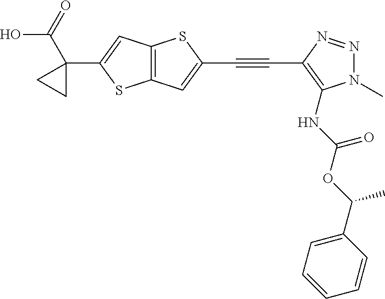 [SMILES: C[C@@H](OC(=O)Nc1c(C#Cc2cc3sc(C4(C(=O)O)CC4)cc3s2)nnn1C)c1ccccc1] |
| IT070 | 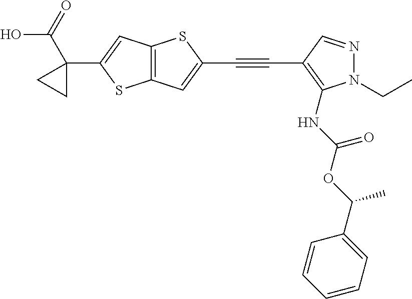 [SMILES: CCn1ncc(C#Cc2cc3sc(C4(C(=O)O)CC4)cc3s2)c1NC(=O)O[C@H](C)c1ccccc1] |
| IT071 | 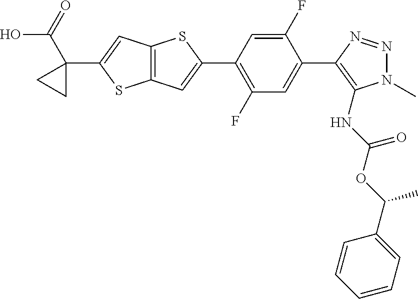 [SMILES: C[C@@H](OC(=O)Nc1c(-c2cc(F)c(-c3cc4sc(C5(C(=O)O)CC5)cc4s3)cc2F)nnn1C)c1ccccc1] |
| IT072 | 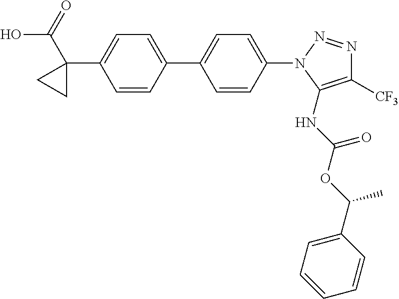 [SMILES: C[C@@H](OC(=O)Nc1c(C(F)(F)F)nnn1-c1ccc(-c2ccc(C3(C(=O)O)CC3)cc2)cc1)c1ccccc1] |
| IT073 | 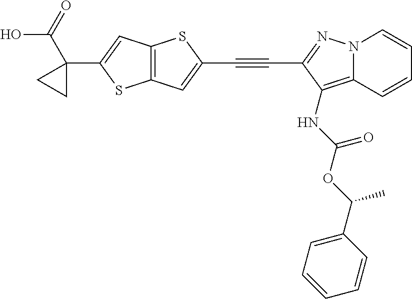 [SMILES: C[C@@H](OC(=O)Nc1c(C#Cc2cc3sc(C4(C(=O)O)CC4)cc3s2)nn2ccccc12)c1ccccc1] |
| IT074 | 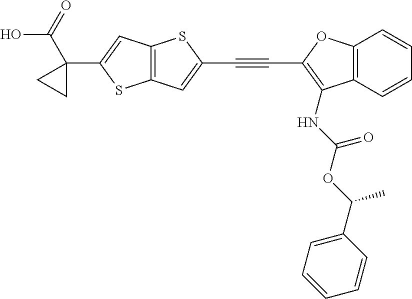 [SMILES: C[C@@H](OC(=O)Nc1c(C#Cc2cc3sc(C4(C(=O)O)CC4)cc3s2)oc2ccccc12)c1ccccc1] |
| IT075 | 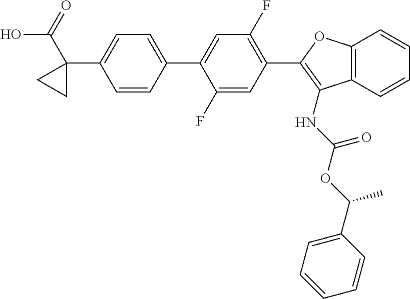 [SMILES: C[C@@H](OC(=O)Nc1c(-c2cc(F)c(-c3ccc(C4(C(=O)O)CC4)cc3)cc2F)oc2ccccc12)c1ccccc1] |
| IT076 | 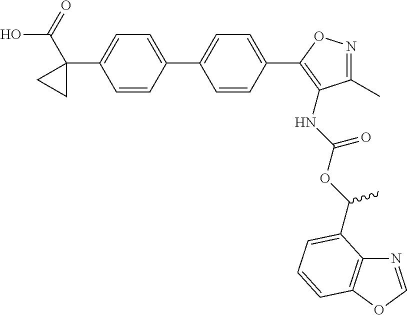 [SMILES: Cc1noc(-c2ccc(-c3ccc(C4(C(=O)O)CC4)cc3)cc2)c1NC(=O)OC(C)c1cccc2ocnc12] |
| | Isomer 1 |
| IT077 | 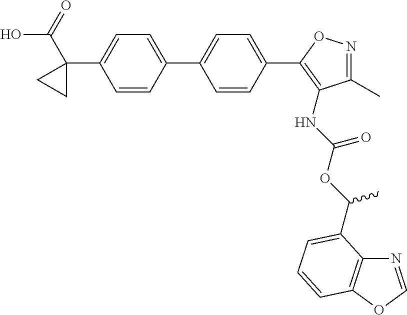 [SMILES: Cc1noc(-c2ccc(-c3ccc(C4(C(=O)O)CC4)cc3)cc2)c1NC(=O)OC(C)c1cccc2ocnc12] |
| | Isomer 2 |
| IT078 | 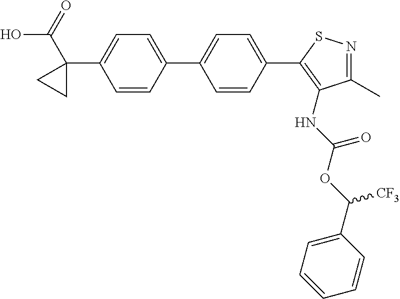 [SMILES: Cc1nsc(-c2ccc(-c3ccc(C4(C(=O)O)CC4)cc3)cc2)c1NC(=O)OC(c1ccccc1)C(F)(F)F] |
| | Isomer 1 |
| IT079 | 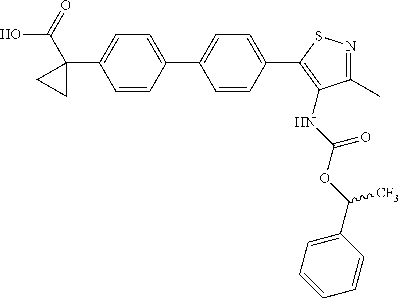 [SMILES: Cc1nsc(-c2ccc(-c3ccc(C4(C(=O)O)CC4)cc3)cc2)c1NC(=O)OC(c1ccccc1)C(F)(F)F] |
| | Isomer 2 |
| IT080 | 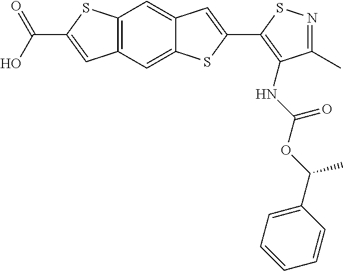 [SMILES: Cc1nsc(-c2cc3cc4sc(C(=O)O)cc4cc3s2)c1NC(=O)O[C@H](C)c1ccccc1] |
| IT081 | 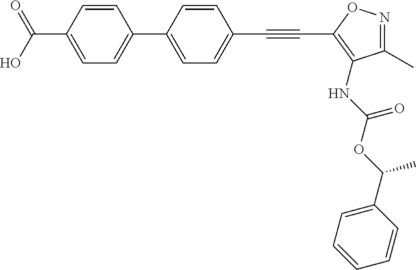 [SMILES: Cc1noc(C#Cc2ccc(-c3ccc(C(=O)O)cc3)cc2)c1NC(=O)O[C@H](C)c1ccccc1] |
| IT082 | 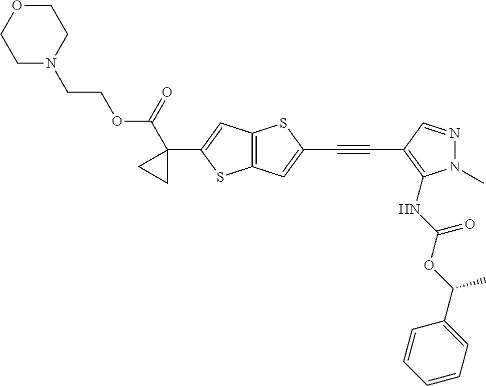 [SMILES: C[C@@H](OC(=O)Nc1c(C#Cc2cc3sc(C4(C(=O)OCCN5CCOCC5)CC4)cc3s2)cnn1C)c1ccccc1] |
| IT083 | 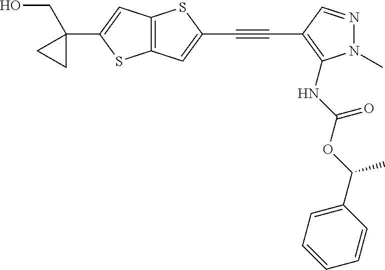 [SMILES: C[C@@H](OC(=O)Nc1c(C#Cc2cc3sc(C4(CO)CC4)cc3s2)cnn1C)c1ccccc1] |
| IT084 | 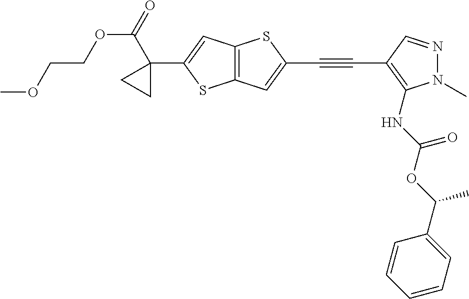 [SMILES: COCCOC(=O)C1(c2cc3sc(C#Cc4cnn(C)c4NC(=O)O[C@H](C)c4ccccc4)cc3s2)CC1] |
| IT085 | 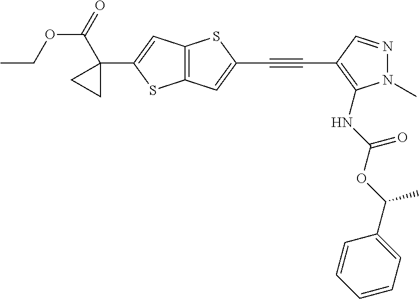 [SMILES: CCOC(=O)C1(c2cc3sc(C#Cc4cnn(C)c4NC(=O)O[C@H](C)c4ccccc4)cc3s2)CC1] |
| IT086 | 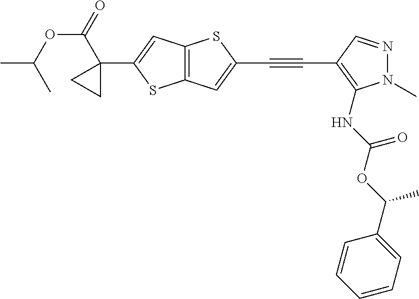 [SMILES: CC(C)OC(=O)C1(c2cc3sc(C#Cc4cnn(C)c4NC(=O)O[C@H](C)c4ccccc4)cc3s2)CC1] |
| IT087 | 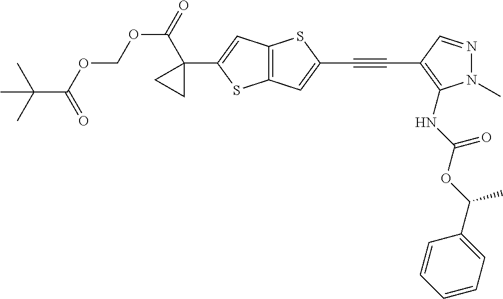 [SMILES: C[C@@H](OC(=O)Nc1c(C#Cc2cc3sc(C4(C(=O)OCOC(=O)C(C)(C)C)CC4)cc3s2)cnn1C)c1ccccc1] |
| IT088 | 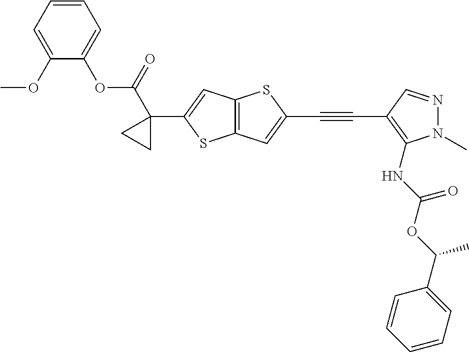 [SMILES: COc1ccccc1OC(=O)C1(c2cc3sc(C#Cc4cnn(C)c4NC(=O)O[C@H](C)c4ccccc4)cc3s2)CC1] |
| IT089 | 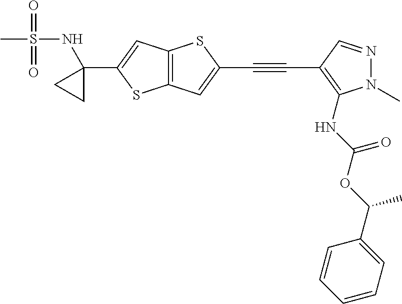 [SMILES: C[C@@H](OC(=O)Nc1c(C#Cc2cc3sc(C4(NS(C)(=O)=O)CC4)cc3s2)cnn1C)c1ccccc1] |
| IT090 | 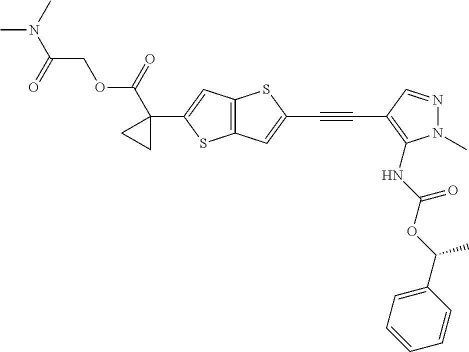 [SMILES: C[C@@H](OC(=O)Nc1c(C#Cc2cc3sc(C4(C(=O)OCC(=O)N(C)C)CC4)cc3s2)cnn1C)c1ccccc1] |
| IT091 | 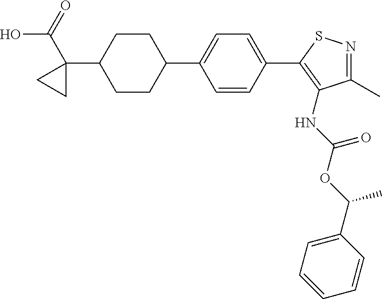 [SMILES: Cc1nsc(-c2ccc(C3CCC(C4(C(=O)O)CC4)CC3)cc2)c1NC(=O)O[C@H](C)c1ccccc1] |
| IT092 | 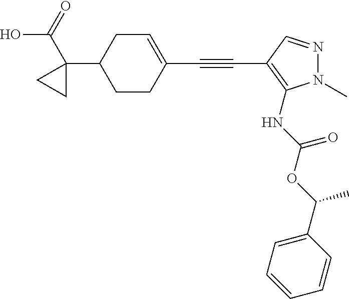 [SMILES: C[C@@H](OC(=O)Nc1c(C#CC2=CCC(C3(C(=O)O)CC3)CC2)cnn1C)c1ccccc1] |
| IT093 | 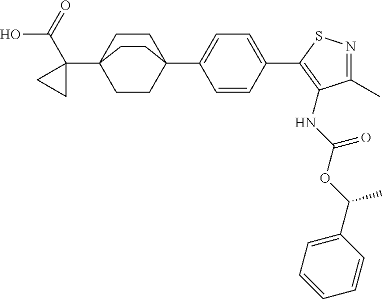 [SMILES: Cc1nsc(-c2ccc(C34CCC(C5(C(=O)O)CC5)(CC3)CC4)cc2)c1NC(=O)O[C@H](C)c1ccccc1] |
| IT094 | 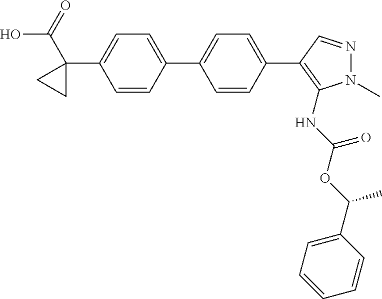 [SMILES: C[C@@H](OC(=O)Nc1c(-c2ccc(-c3ccc(C4(C(=O)O)CC4)cc3)cc2)cnn1C)c1ccccc1] |
| IT095 | 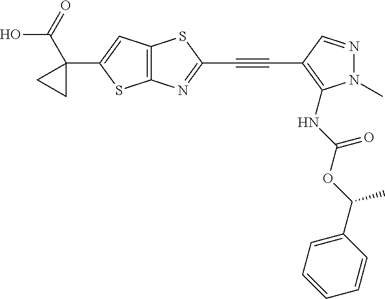 [SMILES: C[C@@H](OC(=O)Nc1c(C#Cc2nc3sc(C4(C(=O)O)CC4)cc3s2)cnn1C)c1ccccc1] |
| IT096 | 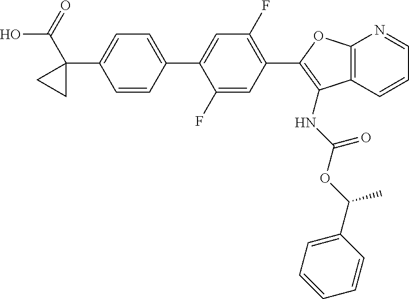 [SMILES: C[C@@H](OC(=O)Nc1c(-c2cc(F)c(-c3ccc(C4(C(=O)O)CC4)cc3)cc2F)oc2ncccc12)c1ccccc1] |
| IT097 | 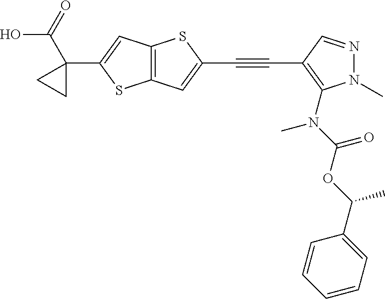 [SMILES: C[C@@H](OC(=O)N(C)c1c(C#Cc2cc3sc(C4(C(=O)O)CC4)cc3s2)cnn1C)c1ccccc1] |
| IT098 | 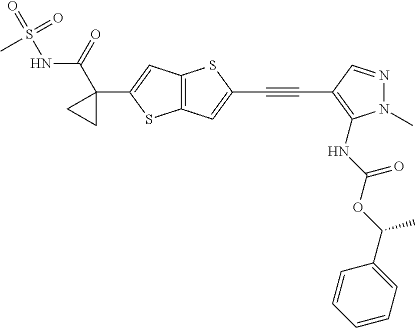 [SMILES: C[C@@H](OC(=O)Nc1c(C#Cc2cc3sc(C4(C(=O)NS(C)(=O)=O)CC4)cc3s2)cnn1C)c1ccccc1] |
| IT099 | 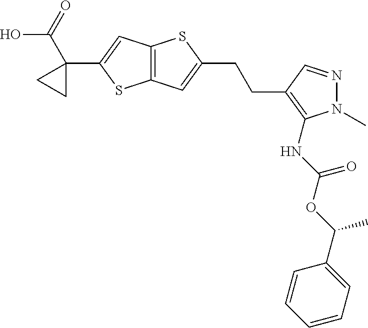 [SMILES: C[C@@H](OC(=O)Nc1c(CCc2cc3sc(C4(C(=O)O)CC4)cc3s2)cnn1C)c1ccccc1] |
| IT100 | 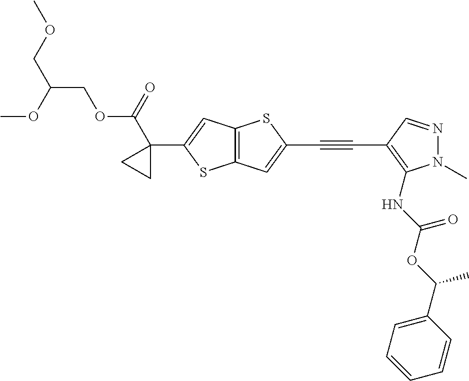 [SMILES: COCC(COC(=O)C1(c2cc3sc(C#Cc4cnn(C)c4NC(=O)O[C@H](C)c4ccccc4)cc3s2)CC1)OC] |
| IT101 | 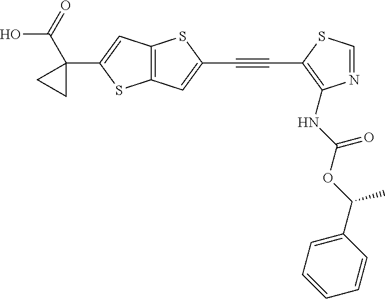 [SMILES: C[C@@H](OC(=O)Nc1ncsc1C#Cc1cc2sc(C3(C(=O)O)CC3)cc2s1)c1ccccc1] |
| IT102 | 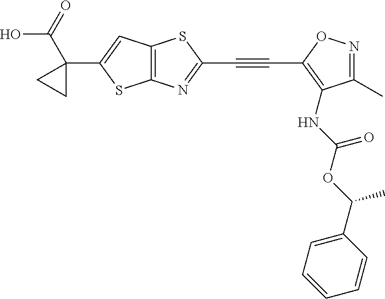 [SMILES: Cc1noc(C#Cc2nc3sc(C4(C(=O)O)CC4)cc3s2)c1NC(=O)O[C@H](C)c1ccccc1] |
| IT103 | 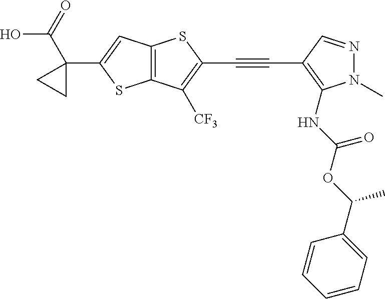 [SMILES: C[C@@H](OC(=O)Nc1c(C#Cc2sc3cc(C4(C(=O)O)CC4)sc3c2C(F)(F)F)cnn1C)c1ccccc1] |
| IT104 | 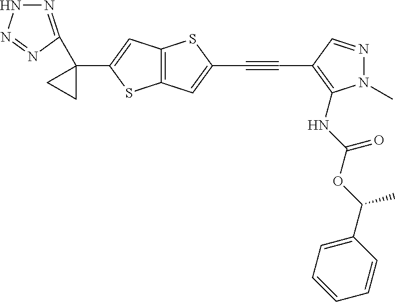 [SMILES: C[C@@H](OC(=O)Nc1c(C#Cc2cc3sc(C4(c5nn[nH]n5)CC4)cc3s2)cnn1C)c1ccccc1] |
| IT105 | 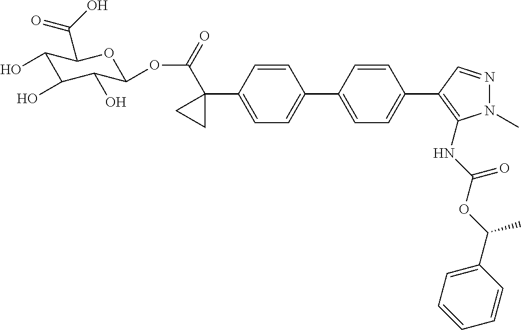 [SMILES: C[C@@H](OC(=O)Nc1c(-c2ccc(-c3ccc(C4(C(=O)OC5OC(C(=O)O)C(O)C(O)C5O)CC4)cc3)cc2)cnn1C)c1ccccc1] |
| IT106 | 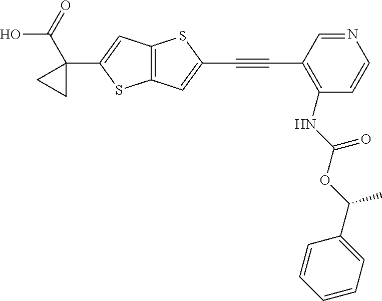 [SMILES: C[C@@H](OC(=O)Nc1ccncc1C#Cc1cc2sc(C3(C(=O)O)CC3)cc2s1)c1ccccc1] |
| IT107 | 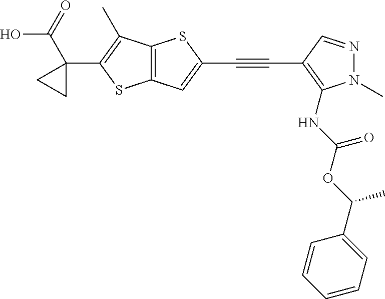 [SMILES: Cc1c(C2(C(=O)O)CC2)sc2cc(C#Cc3cnn(C)c3NC(=O)O[C@H](C)c3ccccc3)sc12] |
| IT108 | 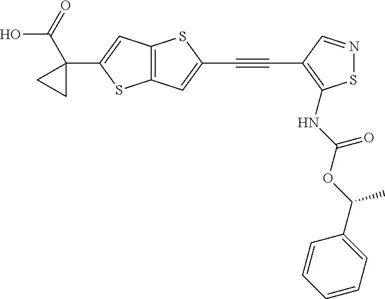 [SMILES: C[C@@H](OC(=O)Nc1sncc1C#Cc1cc2sc(C3(C(=O)O)CC3)cc2s1)c1ccccc1] |
| IT109 | 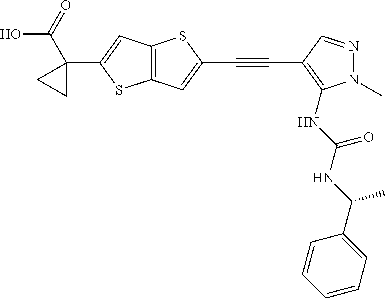 [SMILES: C[C@@H](NC(=O)Nc1c(C#Cc2cc3sc(C4(C(=O)O)CC4)cc3s2)cnn1C)c1ccccc1] |
| IT110 | 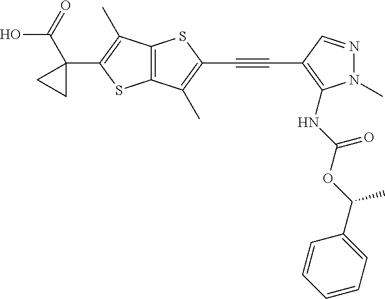 [SMILES: Cc1c(C#Cc2cnn(C)c2NC(=O)O[C@H](C)c2ccccc2)sc2c(C)c(C3(C(=O)O)CC3)sc12] |
| IT111 | 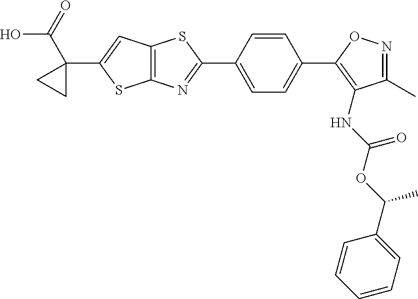 [SMILES: Cc1noc(-c2ccc(-c3nc4sc(C5(C(=O)O)CC5)cc4s3)cc2)c1NC(=O)O[C@H](C)c1ccccc1] |
| IT112 | 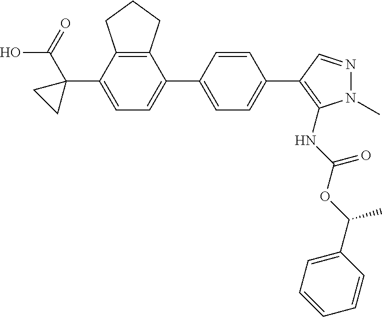 [SMILES: C[C@@H](OC(=O)Nc1c(-c2ccc(-c3ccc(C4(C(=O)O)CC4)c4c3CCC4)cc2)cnn1C)c1ccccc1] |
| IT113 | 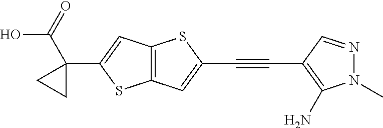 [SMILES: Cn1ncc(C#Cc2cc3sc(C4(C(=O)O)CC4)cc3s2)c1N] |
| IT114 | 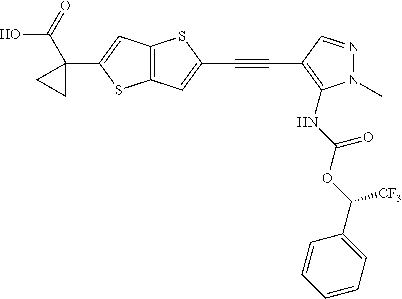 [SMILES: Cn1ncc(C#Cc2cc3sc(C4(C(=O)O)CC4)cc3s2)c1NC(=O)O[C@@H](c1ccccc1)C(F)(F)F] |
| IT115 | 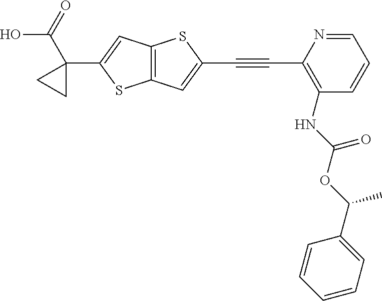 [SMILES: C[C@@H](OC(=O)Nc1cccnc1C#Cc1cc2sc(C3(C(=O)O)CC3)cc2s1)c1ccccc1] |
| IT116 | 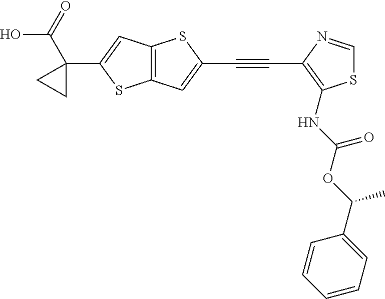 [SMILES: C[C@@H](OC(=O)Nc1scnc1C#Cc1cc2sc(C3(C(=O)O)CC3)cc2s1)c1ccccc1] |
| IT117 | 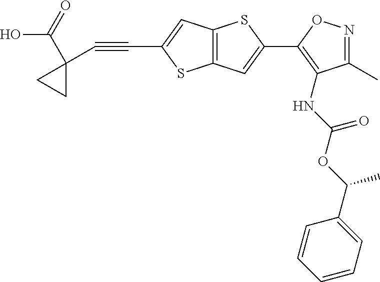 [SMILES: Cc1noc(-c2cc3sc(C#CC4(C(=O)O)CC4)cc3s2)c1NC(=O)O[C@H](C)c1ccccc1] |
| IT118 | 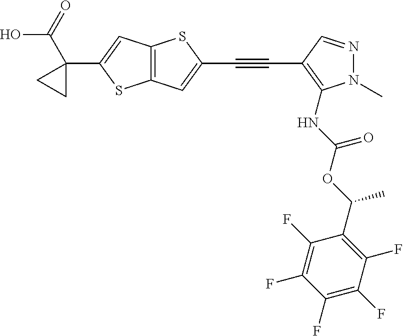 [SMILES: C[C@@H](OC(=O)Nc1c(C#Cc2cc3sc(C4(C(=O)O)CC4)cc3s2)cnn1C)c1c(F)c(F)c(F)c(F)c1F] |
| IT119 | 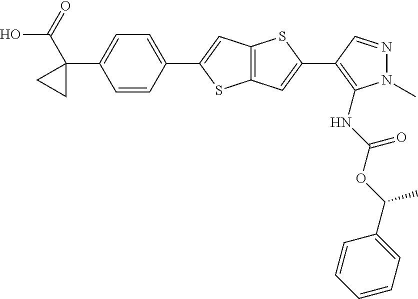 [SMILES: C[C@@H](OC(=O)Nc1c(-c2cc3sc(-c4ccc(C5(C(=O)O)CC5)cc4)cc3s2)cnn1C)c1ccccc1] |
| IT120 | 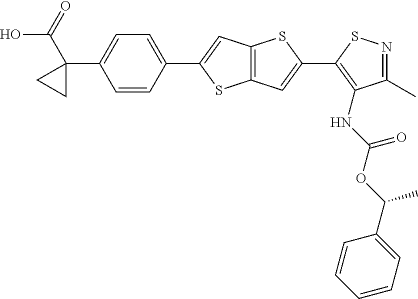 [SMILES: Cc1nsc(-c2cc3sc(-c4ccc(C5(C(=O)O)CC5)cc4)cc3s2)c1NC(=O)O[C@H](C)c1ccccc1] |
| IT121 | 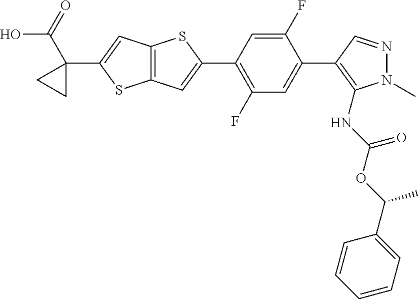 [SMILES: C[C@@H](OC(=O)Nc1c(-c2cc(F)c(-c3cc4sc(C5(C(=O)O)CC5)cc4s3)cc2F)cnn1C)c1ccccc1] |
| IT122 | 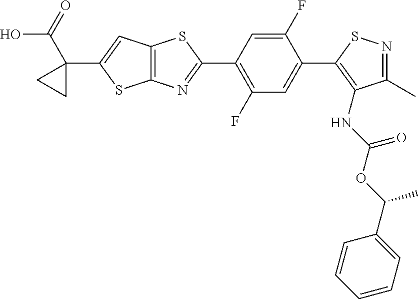 [SMILES: Cc1nsc(-c2cc(F)c(-c3nc4sc(C5(C(=O)O)CC5)cc4s3)cc2F)c1NC(=O)O[C@H](C)c1ccccc1] |
| IT123 | 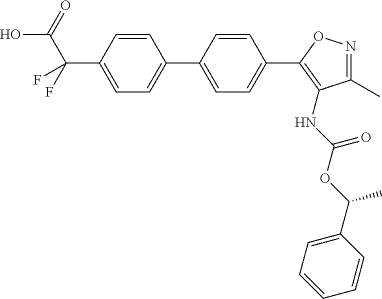 [SMILES: Cc1noc(-c2ccc(-c3ccc(C(F)(F)C(=O)O)cc3)cc2)c1NC(=O)O[C@H](C)c1ccccc1] |
| IT124 | 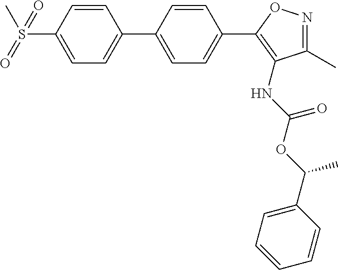 [SMILES: Cc1noc(-c2ccc(-c3ccc(S(C)(=O)=O)cc3)cc2)c1NC(=O)O[C@H](C)c1ccccc1] |
| IT125 | 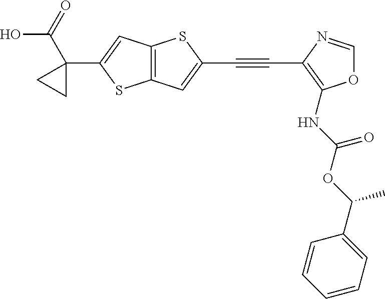 [SMILES: C[C@@H](OC(=O)Nc1ocnc1C#Cc1cc2sc(C3(C(=O)O)CC3)cc2s1)c1ccccc1] |
| IT126 | 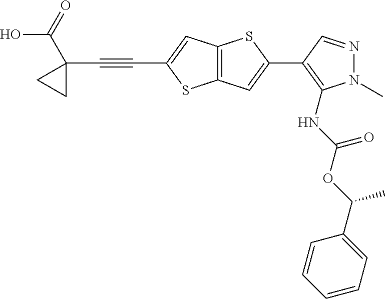 [SMILES: C[C@@H](OC(=O)Nc1c(-c2cc3sc(C#CC4(C(=O)O)CC4)cc3s2)cnn1C)c1ccccc1] |
| IT127 | 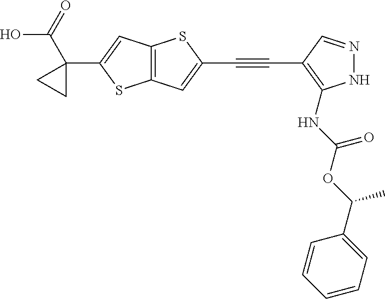 [SMILES: C[C@@H](OC(=O)Nc1[nH]ncc1C#Cc1cc2sc(C3(C(=O)O)CC3)cc2s1)c1ccccc1] |
| IT128 | 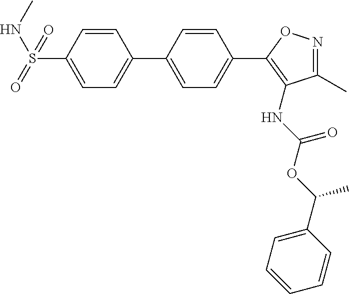 [SMILES: CNS(=O)(=O)c1ccc(-c2ccc(-c3onc(C)c3NC(=O)O[C@H](C)c3ccccc3)cc2)cc1] |
| IT129 | 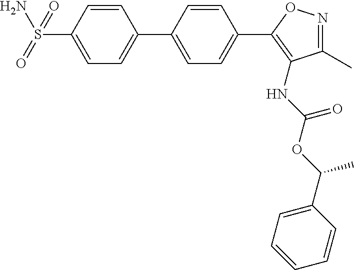 [SMILES: Cc1noc(-c2ccc(-c3ccc(S(N)(=O)=O)cc3)cc2)c1NC(=O)O[C@H](C)c1ccccc1] |
| IT130 | 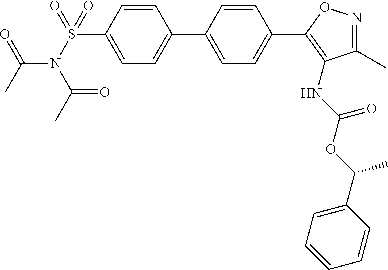 [SMILES: CC(=O)N(C(C)=O)S(=O)(=O)c1ccc(-c2ccc(-c3onc(C)c3NC(=O)O[C@H](C)c3ccccc3)cc2)cc1] |
| IT131 | 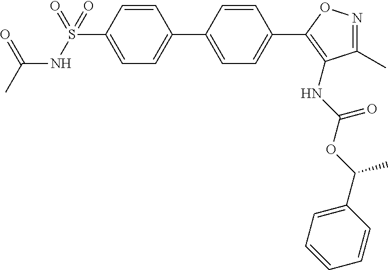 [SMILES: CC(=O)NS(=O)(=O)c1ccc(-c2ccc(-c3onc(C)c3NC(=O)O[C@H](C)c3ccccc3)cc2)cc1] |
| IT132 | 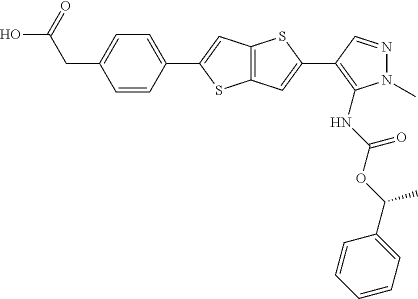 [SMILES: C[C@@H](OC(=O)Nc1c(-c2cc3sc(-c4ccc(CC(=O)O)cc4)cc3s2)cnn1C)c1ccccc1] |
| IT133 | 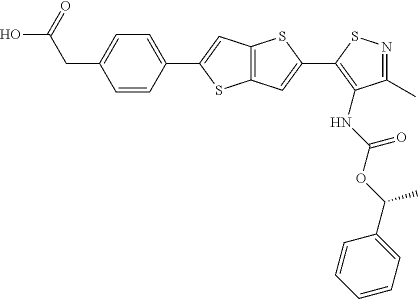 [SMILES: Cc1nsc(-c2cc3sc(-c4ccc(CC(=O)O)cc4)cc3s2)c1NC(=O)O[C@H](C)c1ccccc1] |
| IT134 | 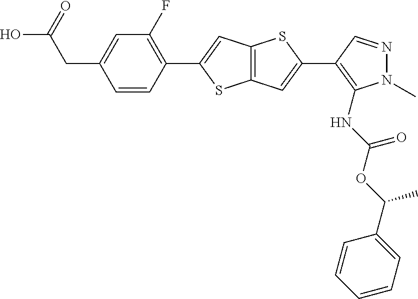 [SMILES: C[C@@H](OC(=O)Nc1c(-c2cc3sc(-c4ccc(CC(=O)O)cc4F)cc3s2)cnn1C)c1ccccc1] |
| IT135 | 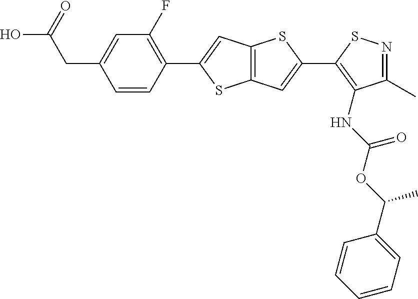 [SMILES: Cc1nsc(-c2cc3sc(-c4ccc(CC(=O)O)cc4F)cc3s2)c1NC(=O)O[C@H](C)c1ccccc1] |
| IT136 | 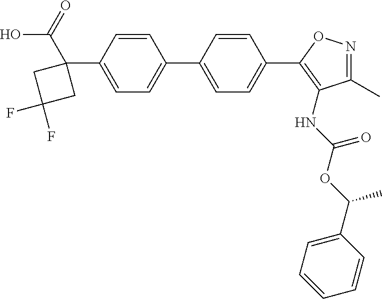 [SMILES: Cc1noc(-c2ccc(-c3ccc(C4(C(=O)O)CC(F)(F)C4)cc3)cc2)c1NC(=O)O[C@H](C)c1ccccc1] |
| IT137 | 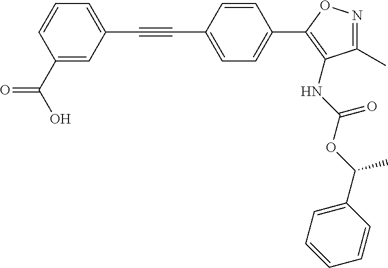 [SMILES: Cc1noc(-c2ccc(C#Cc3cccc(C(=O)O)c3)cc2)c1NC(=O)O[C@H](C)c1ccccc1] |
| IT138 | 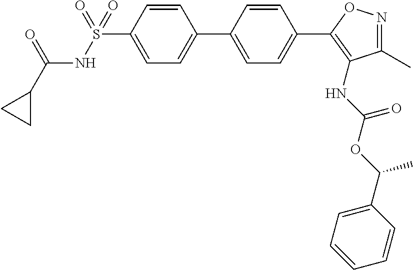 [SMILES: Cc1noc(-c2ccc(-c3ccc(S(=O)(=O)NC(=O)C4CC4)cc3)cc2)c1NC(=O)O[C@H](C)c1ccccc1] |
| IT139 | 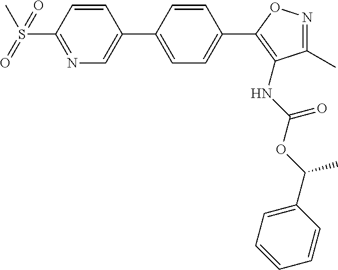 [SMILES: Cc1noc(-c2ccc(-c3ccc(S(C)(=O)=O)nc3)cc2)c1NC(=O)O[C@H](C)c1ccccc1] |
| IT140 | 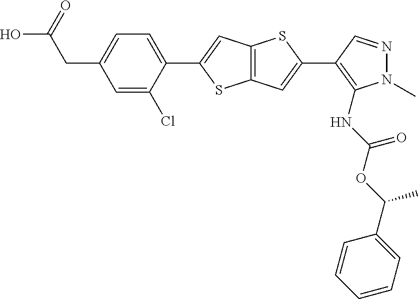 [SMILES: C[C@@H](OC(=O)Nc1c(-c2cc3sc(-c4ccc(CC(=O)O)cc4Cl)cc3s2)cnn1C)c1ccccc1] |
| IT141 | 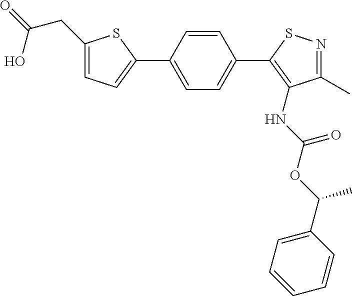 [SMILES: Cc1nsc(-c2ccc(-c3ccc(CC(=O)O)s3)cc2)c1NC(=O)O[C@H](C)c1ccccc1] |
| IT142 | 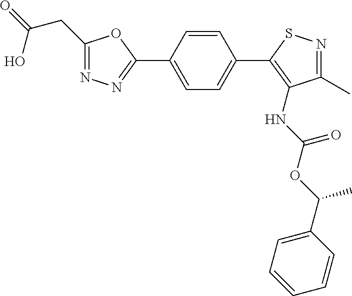 [SMILES: Cc1nsc(-c2ccc(-c3nnc(CC(=O)O)o3)cc2)c1NC(=O)O[C@H](C)c1ccccc1] |
| IT143 | 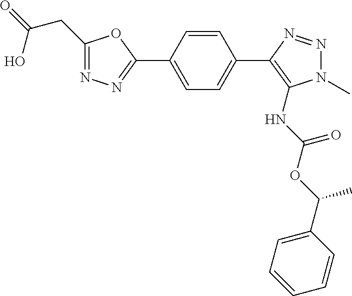 [SMILES: C[C@@H](OC(=O)Nc1c(-c2ccc(-c3nnc(CC(=O)O)o3)cc2)nnn1C)c1ccccc1] |
| IT144 | 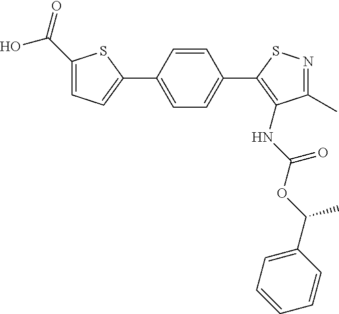 [SMILES: Cc1nsc(-c2ccc(-c3ccc(C(=O)O)s3)cc2)c1NC(=O)O[C@H](C)c1ccccc1] |
| IT145 | 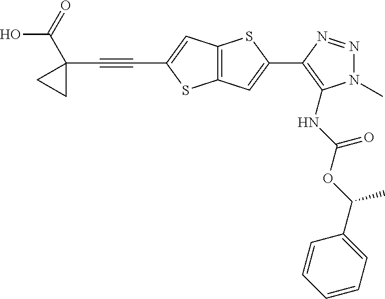 [SMILES: C[C@@H](OC(=O)Nc1c(-c2cc3sc(C#CC4(C(=O)O)CC4)cc3s2)nnn1C)c1ccccc1] |
| IT146 | 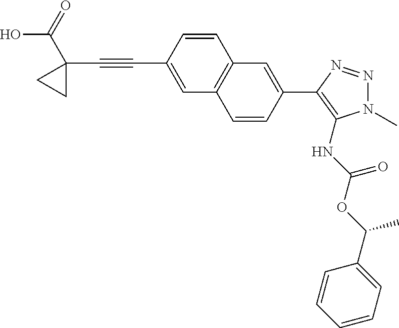 [SMILES: C[C@@H](OC(=O)Nc1c(-c2ccc3cc(C#CC4(C(=O)O)CC4)ccc3c2)nnn1C)c1ccccc1] |
| IT147 | 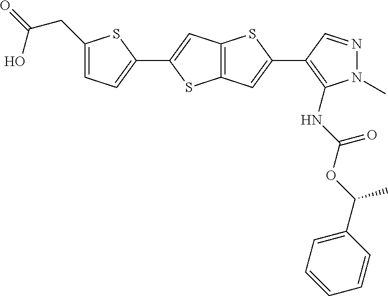 [SMILES: C[C@@H](OC(=O)Nc1c(-c2cc3sc(-c4ccc(CC(=O)O)s4)cc3s2)cnn1C)c1ccccc1] |
| IT148 | 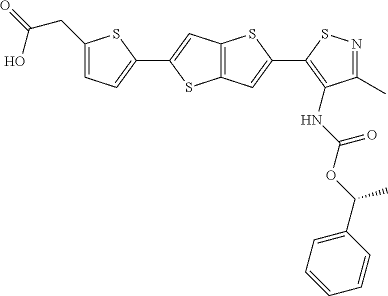 [SMILES: Cc1nsc(-c2cc3sc(-c4ccc(CC(=O)O)s4)cc3s2)c1NC(=O)O[C@H](C)c1ccccc1] |
| IT149 | 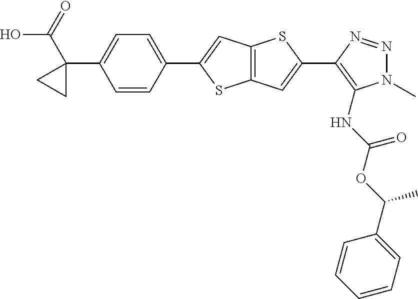 [SMILES: C[C@@H](OC(=O)Nc1c(-c2cc3sc(-c4ccc(C5(C(=O)O)CC5)cc4)cc3s2)nnn1C)c1ccccc1] |
| IT150 | 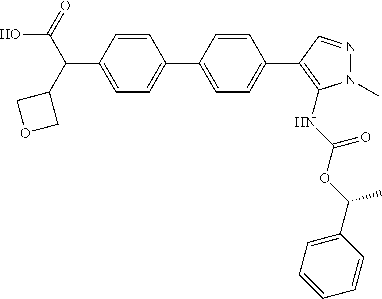 [SMILES: C[C@@H](OC(=O)Nc1c(-c2ccc(-c3ccc(C(C(=O)O)C4COC4)cc3)cc2)cnn1C)c1ccccc1] |
| IT151 | 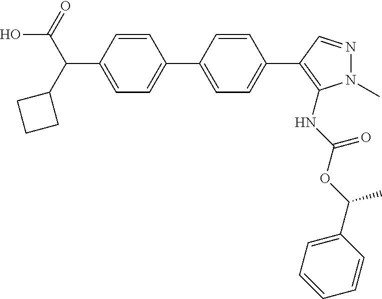 [SMILES: C[C@@H](OC(=O)Nc1c(-c2ccc(-c3ccc(C(C(=O)O)C4CCC4)cc3)cc2)cnn1C)c1ccccc1] |
| IT152 | 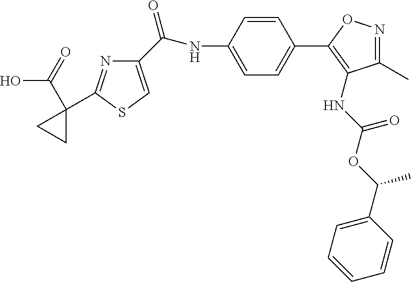 [SMILES: Cc1noc(-c2ccc(NC(=O)c3csc(C4(C(=O)O)CC4)n3)cc2)c1NC(=O)O[C@H](C)c1ccccc1] |
| IT153 | 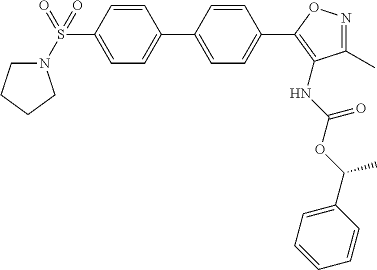 [SMILES: Cc1noc(-c2ccc(-c3ccc(S(=O)(=O)N4CCCC4)cc3)cc2)c1NC(=O)O[C@H](C)c1ccccc1] |
| IT154 | 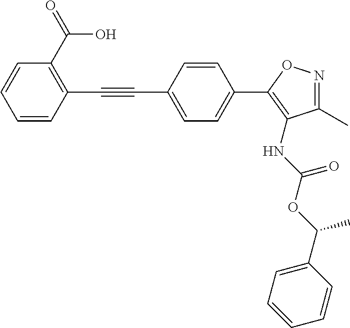 [SMILES: Cc1noc(-c2ccc(C#Cc3ccccc3C(=O)O)cc2)c1NC(=O)O[C@H](C)c1ccccc1] |
| IT155 | 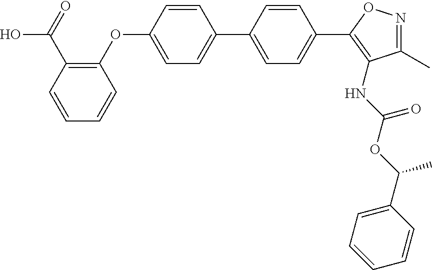 [SMILES: Cc1noc(-c2ccc(-c3ccc(Oc4ccccc4C(=O)O)cc3)cc2)c1NC(=O)O[C@H](C)c1ccccc1] |
| IT156 | 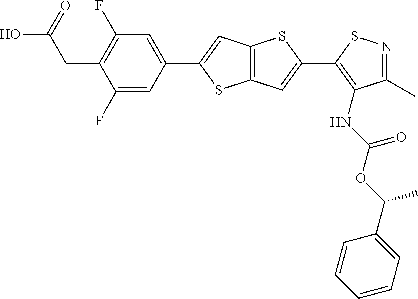 [SMILES: Cc1nsc(-c2cc3sc(-c4cc(F)c(CC(=O)O)c(F)c4)cc3s2)c1NC(=O)O[C@H](C)c1ccccc1] |
| IT157 | 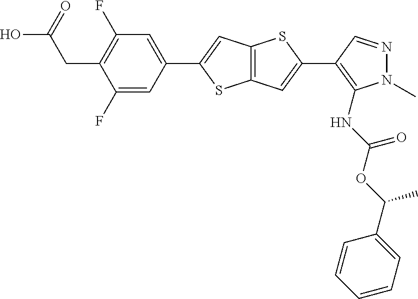 [SMILES: C[C@@H](OC(=O)Nc1c(-c2cc3sc(-c4cc(F)c(CC(=O)O)c(F)c4)cc3s2)cnn1C)c1ccccc1] |
| IT158 | 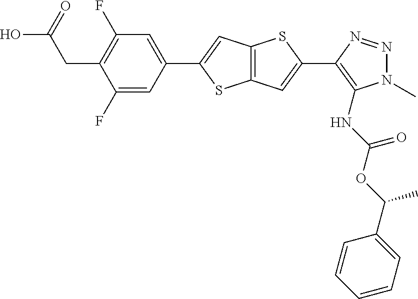 [SMILES: C[C@@H](OC(=O)Nc1c(-c2cc3sc(-c4cc(F)c(CC(=O)O)c(F)c4)cc3s2)nnn1C)c1ccccc1] |
| IT159 | 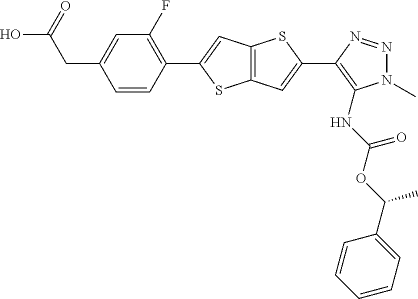 [SMILES: C[C@@H](OC(=O)Nc1c(-c2cc3sc(-c4ccc(CC(=O)O)cc4F)cc3s2)nnn1C)c1ccccc1] |
| IT160 | 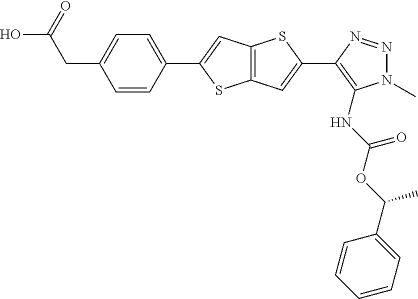 [SMILES: C[C@@H](OC(=O)Nc1c(-c2cc3sc(-c4ccc(CC(=O)O)cc4)cc3s2)nnn1C)c1ccccc1] |
| IT161 | 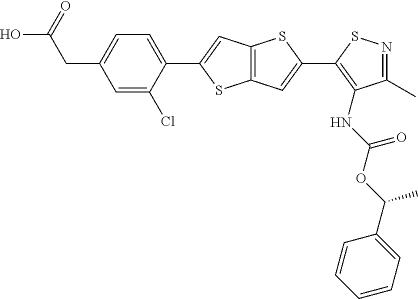 [SMILES: Cc1nsc(-c2cc3sc(-c4ccc(CC(=O)O)cc4Cl)cc3s2)c1NC(=O)O[C@H](C)c1ccccc1] |
| IT162 | 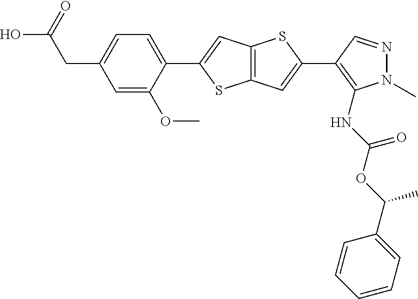 [SMILES: COc1cc(CC(=O)O)ccc1-c1cc2sc(-c3cnn(C)c3NC(=O)O[C@H](C)c3ccccc3)cc2s1] |
| IT163 | 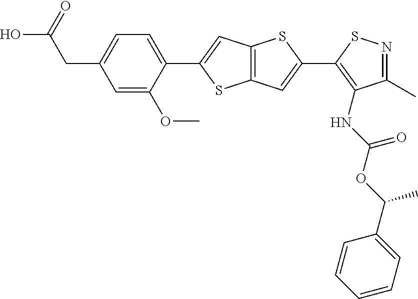 [SMILES: COc1cc(CC(=O)O)ccc1-c1cc2sc(-c3snc(C)c3NC(=O)O[C@H](C)c3ccccc3)cc2s1] |
| IT164 | 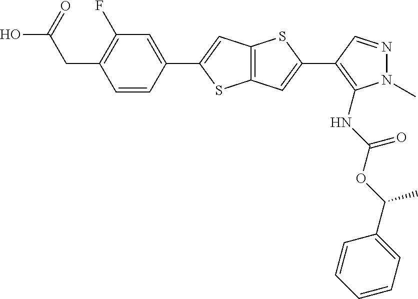 [SMILES: C[C@@H](OC(=O)Nc1c(-c2cc3sc(-c4ccc(CC(=O)O)c(F)c4)cc3s2)cnn1C)c1ccccc1] |
| IT165 | 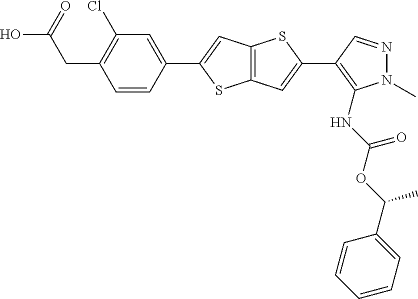 [SMILES: C[C@@H](OC(=O)Nc1c(-c2cc3sc(-c4ccc(CC(=O)O)c(Cl)c4)cc3s2)cnn1C)c1ccccc1] |
| IT166 | 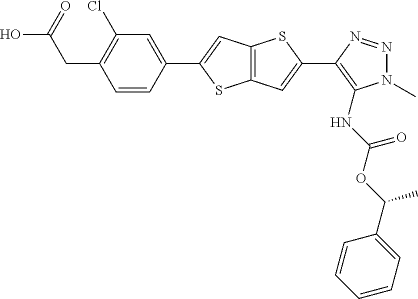 [SMILES: C[C@@H](OC(=O)Nc1c(-c2cc3sc(-c4ccc(CC(=O)O)c(Cl)c4)cc3s2)nnn1C)c1ccccc1] |
| IT167 | 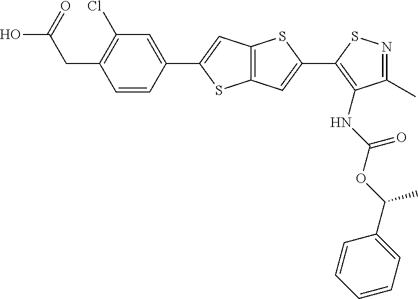 [SMILES: Cc1nsc(-c2cc3sc(-c4ccc(CC(=O)O)c(Cl)c4)cc3s2)c1NC(=O)O[C@H](C)c1ccccc1] |
| IT168 | 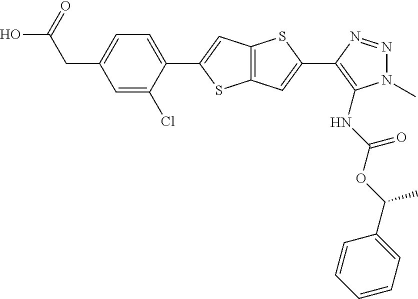 [SMILES: C[C@@H](OC(=O)Nc1c(-c2cc3sc(-c4ccc(CC(=O)O)cc4Cl)cc3s2)nnn1C)c1ccccc1] |
| IT169 | 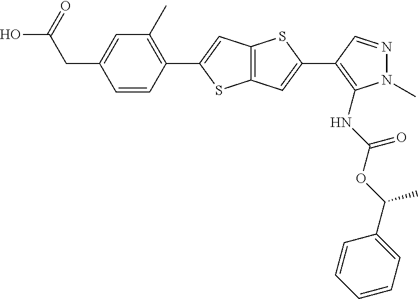 [SMILES: Cc1cc(CC(=O)O)ccc1-c1cc2sc(-c3cnn(C)c3NC(=O)O[C@H](C)c3ccccc3)cc2s1] |
| IT170 | 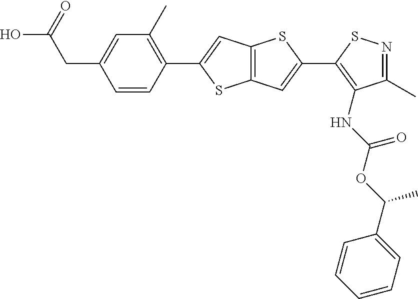 [SMILES: Cc1cc(CC(=O)O)ccc1-c1cc2sc(-c3snc(C)c3NC(=O)O[C@H](C)c3ccccc3)cc2s1] |
| IT171 | 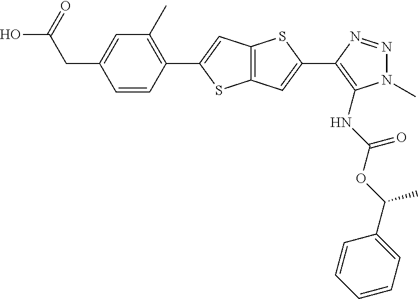 [SMILES: Cc1cc(CC(=O)O)ccc1-c1cc2sc(-c3nnn(C)c3NC(=O)O[C@H](C)c3ccccc3)cc2s1] |
| IT172 | 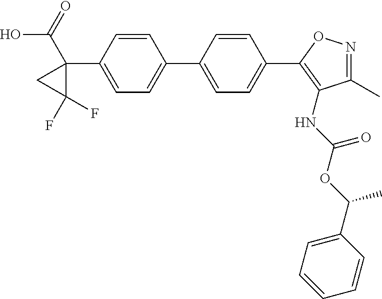 [SMILES: Cc1noc(-c2ccc(-c3ccc(C4(C(=O)O)CC4(F)F)cc3)cc2)c1NC(=O)O[C@H](C)c1ccccc1] |
| IT173 | 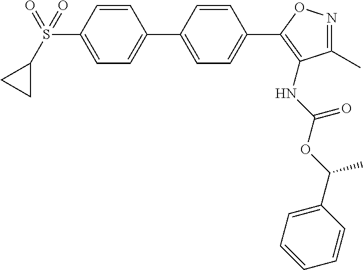 [SMILES: Cc1noc(-c2ccc(-c3ccc(S(=O)(=O)C4CC4)cc3)cc2)c1NC(=O)O[C@H](C)c1ccccc1] |
| IT174 | 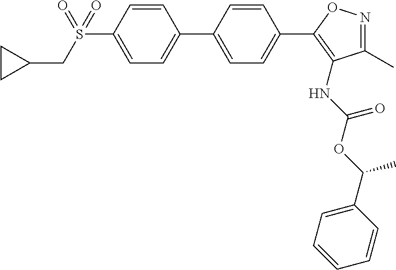 [SMILES: Cc1noc(-c2ccc(-c3ccc(S(=O)(=O)CC4CC4)cc3)cc2)c1NC(=O)O[C@H](C)c1ccccc1] |
| IT175 | 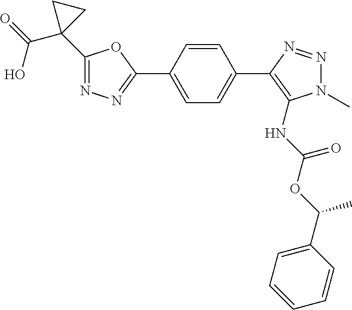 [SMILES: C[C@@H](OC(=O)Nc1c(-c2ccc(-c3nnc(C4(C(=O)O)CC4)o3)cc2)nnn1C)c1ccccc1] |
| IT176 | 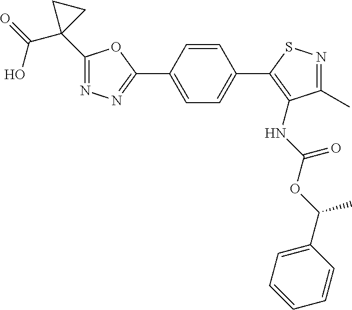 [SMILES: Cc1nsc(-c2ccc(-c3nnc(C4(C(=O)O)CC4)o3)cc2)c1NC(=O)O[C@H](C)c1ccccc1] |
| IT177 | 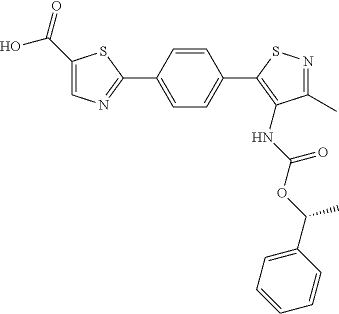 [SMILES: Cc1nsc(-c2ccc(-c3ncc(C(=O)O)s3)cc2)c1NC(=O)O[C@H](C)c1ccccc1] |
| IT178 | 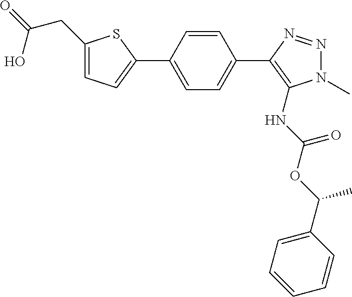 [SMILES: C[C@@H](OC(=O)Nc1c(-c2ccc(-c3ccc(CC(=O)O)s3)cc2)nnn1C)c1ccccc1] |
| IT179 | 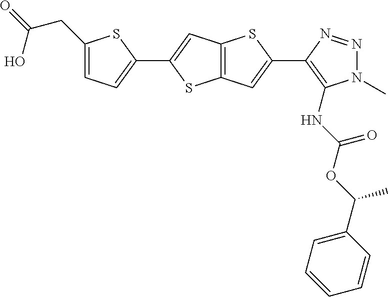 [SMILES: C[C@@H](OC(=O)Nc1c(-c2cc3sc(-c4ccc(CC(=O)O)s4)cc3s2)nnn1C)c1ccccc1] |
| IT180 | 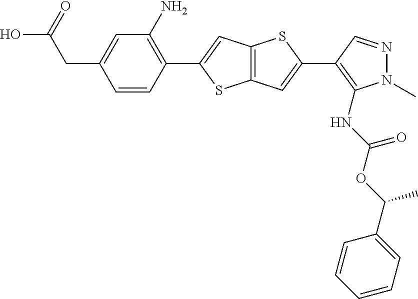 [SMILES: C[C@@H](OC(=O)Nc1c(-c2cc3sc(-c4ccc(CC(=O)O)cc4N)cc3s2)cnn1C)c1ccccc1] |
| IT181 | 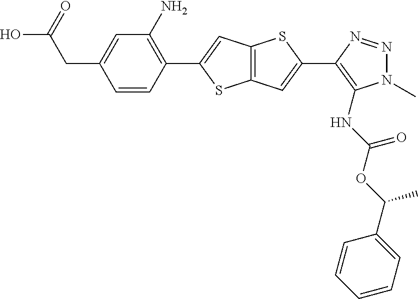 [SMILES: C[C@@H](OC(=O)Nc1c(-c2cc3sc(-c4ccc(CC(=O)O)cc4N)cc3s2)nnn1C)c1ccccc1] |
| IT182 | 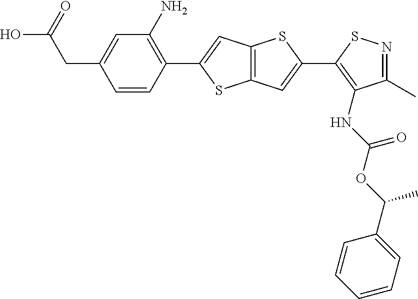 [SMILES: Cc1nsc(-c2cc3sc(-c4ccc(CC(=O)O)cc4N)cc3s2)c1NC(=O)O[C@H](C)c1ccccc1] |
| IT183 | 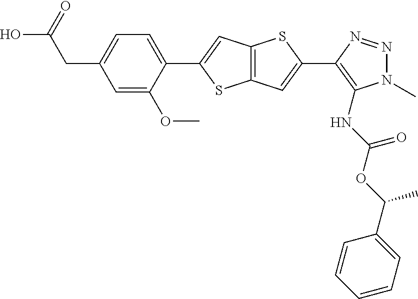 [SMILES: COc1cc(CC(=O)O)ccc1-c1cc2sc(-c3nnn(C)c3NC(=O)O[C@H](C)c3ccccc3)cc2s1] |
| IT184 | 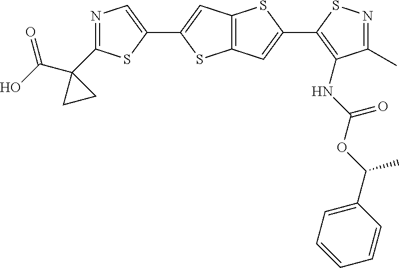 [SMILES: Cc1nsc(-c2cc3sc(-c4cnc(C5(C(=O)O)CC5)s4)cc3s2)c1NC(=O)O[C@H](C)c1ccccc1] |
| IT185 | 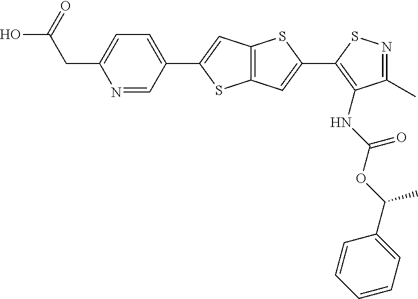 [SMILES: Cc1nsc(-c2cc3sc(-c4ccc(CC(=O)O)nc4)cc3s2)c1NC(=O)O[C@H](C)c1ccccc1] |
| IT186 | 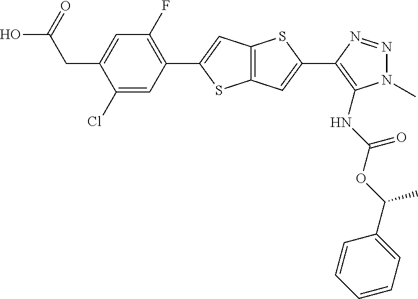 [SMILES: C[C@@H](OC(=O)Nc1c(-c2cc3sc(-c4cc(Cl)c(CC(=O)O)cc4F)cc3s2)nnn1C)c1ccccc1] |
| IT187 | 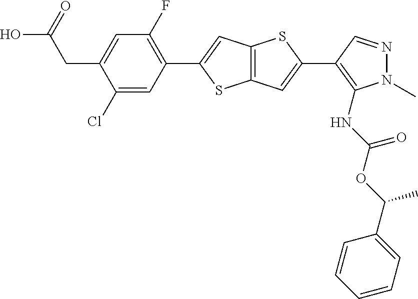 [SMILES: C[C@@H](OC(=O)Nc1c(-c2cc3sc(-c4cc(Cl)c(CC(=O)O)cc4F)cc3s2)cnn1C)c1ccccc1] |
| IT188 | 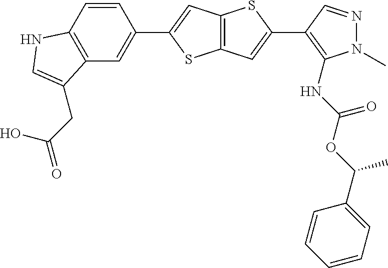 [SMILES: C[C@@H](OC(=O)Nc1c(-c2cc3sc(-c4ccc5[nH]cc(CC(=O)O)c5c4)cc3s2)cnn1C)c1ccccc1] |
| IT189 | 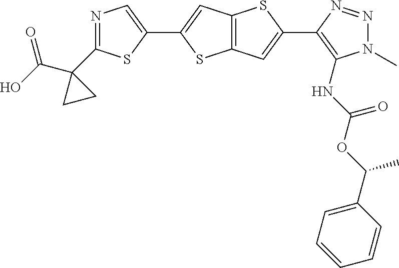 [SMILES: C[C@@H](OC(=O)Nc1c(-c2cc3sc(-c4cnc(C5(C(=O)O)CC5)s4)cc3s2)nnn1C)c1ccccc1] |
| IT190 | 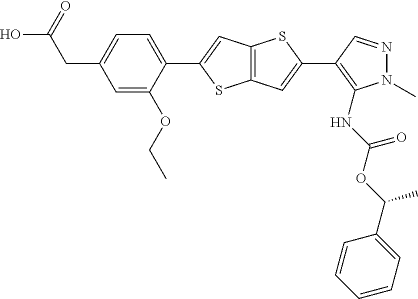 [SMILES: CCOc1cc(CC(=O)O)ccc1-c1cc2sc(-c3cnn(C)c3NC(=O)O[C@H](C)c3ccccc3)cc2s1] |
| IT191 | 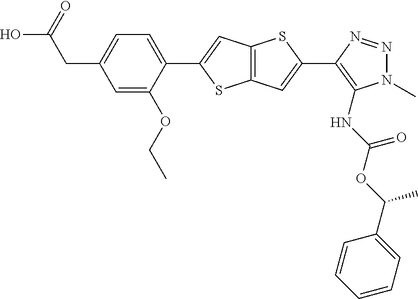 [SMILES: CCOc1cc(CC(=O)O)ccc1-c1cc2sc(-c3nnn(C)c3NC(=O)O[C@H](C)c3ccccc3)cc2s1] |
| IT192 | 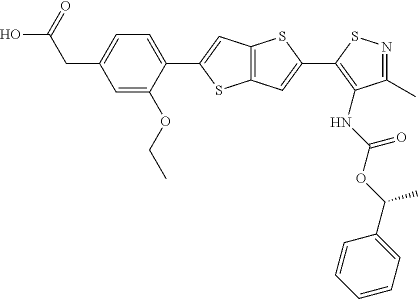 [SMILES: CCOc1cc(CC(=O)O)ccc1-c1cc2sc(-c3snc(C)c3NC(=O)O[C@H](C)c3ccccc3)cc2s1] |
| IT193 | 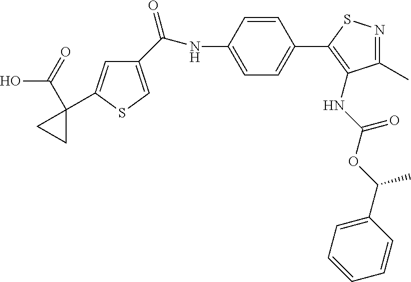 [SMILES: Cc1nsc(-c2ccc(NC(=O)c3csc(C4(C(=O)O)CC4)c3)cc2)c1NC(=O)O[C@H](C)c1ccccc1] |
| IT194 | 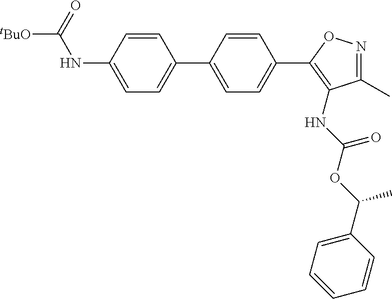 [SMILES: Cc1noc(-c2ccc(-c3ccc(NC(=O)OC(C)(C)C)cc3)cc2)c1NC(=O)O[C@H](C)c1ccccc1] |
| IT195 | 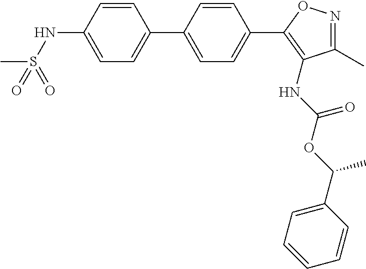 [SMILES: Cc1noc(-c2ccc(-c3ccc(NS(C)(=O)=O)cc3)cc2)c1NC(=O)O[C@H](C)c1ccccc1] |
| IT196 | 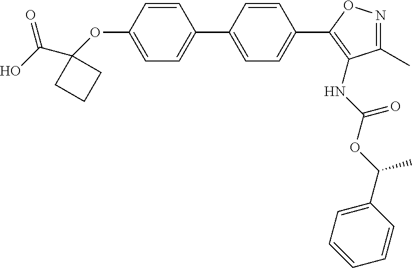 [SMILES: Cc1noc(-c2ccc(-c3ccc(OC4(C(=O)O)CCC4)cc3)cc2)c1NC(=O)O[C@H](C)c1ccccc1] |
| IT197 | 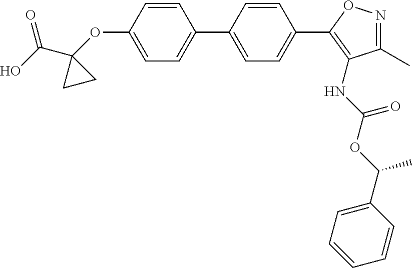 [SMILES: Cc1noc(-c2ccc(-c3ccc(OC4(C(=O)O)CC4)cc3)cc2)c1NC(=O)O[C@H](C)c1ccccc1] |
| IT198 | 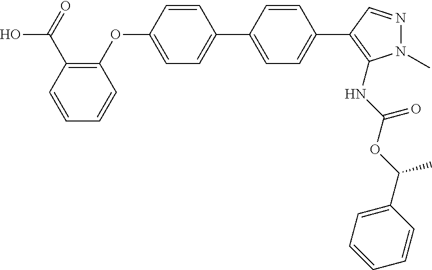 [SMILES: C[C@@H](OC(=O)Nc1c(-c2ccc(-c3ccc(Oc4ccccc4C(=O)O)cc3)cc2)cnn1C)c1ccccc1] |
| IT199 | 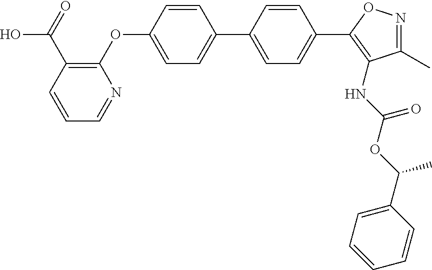 [SMILES: Cc1noc(-c2ccc(-c3ccc(Oc4ncccc4C(=O)O)cc3)cc2)c1NC(=O)O[C@H](C)c1ccccc1] |
| IT200 | 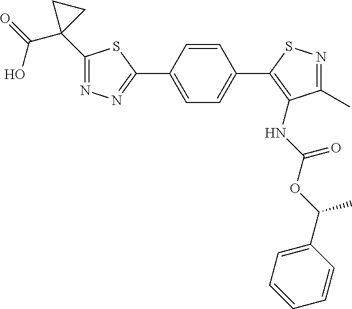 [SMILES: Cc1nsc(-c2ccc(-c3nnc(C4(C(=O)O)CC4)s3)cc2)c1NC(=O)O[C@H](C)c1ccccc1] |
| IT201 | 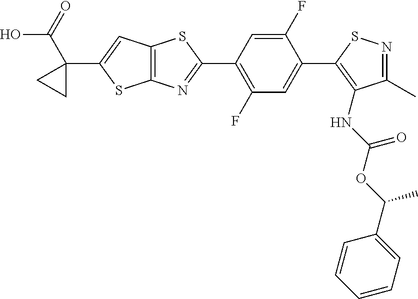 [SMILES: Cc1nsc(-c2cc(F)c(-c3nc4sc(C5(C(=O)O)CC5)cc4s3)cc2F)c1NC(=O)O[C@H](C)c1ccccc1] |
| IT202 | 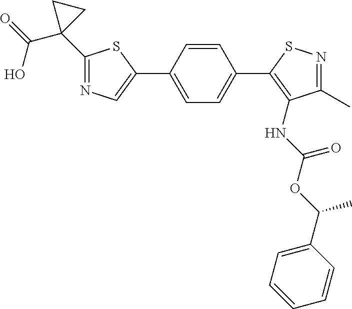 [SMILES: Cc1nsc(-c2ccc(-c3cnc(C4(C(=O)O)CC4)s3)cc2)c1NC(=O)O[C@H](C)c1ccccc1] |
| IT203 | 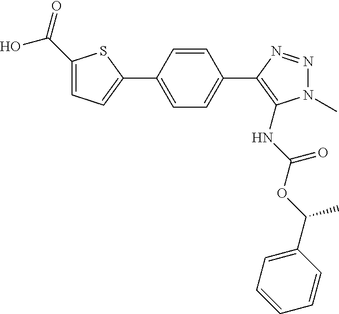 [SMILES: C[C@@H](OC(=O)Nc1c(-c2ccc(-c3ccc(C(=O)O)s3)cc2)nnn1C)c1ccccc1] |
| IT204 | 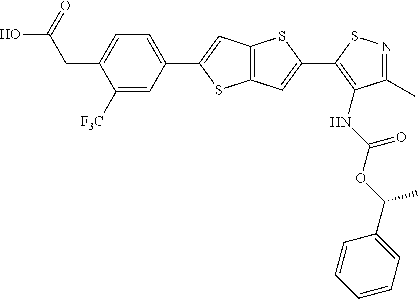 [SMILES: Cc1nsc(-c2cc3sc(-c4ccc(CC(=O)O)c(C(F)(F)F)c4)cc3s2)c1NC(=O)O[C@H](C)c1ccccc1] |
| IT205 | 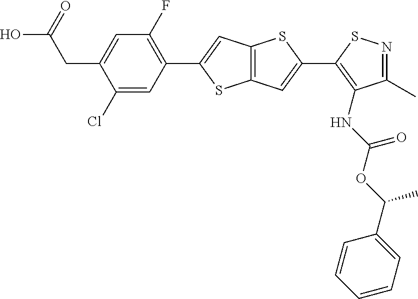 [SMILES: Cc1nsc(-c2cc3sc(-c4cc(Cl)c(CC(=O)O)cc4F)cc3s2)c1NC(=O)O[C@H](C)c1ccccc1] |
| IT206 | 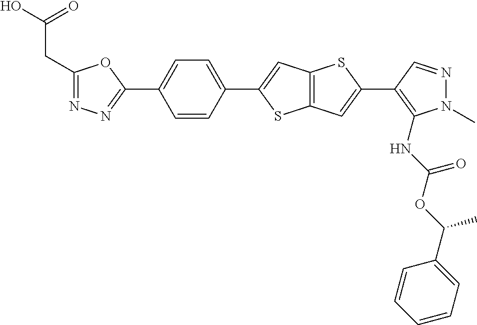 [SMILES: C[C@@H](OC(=O)Nc1c(-c2cc3sc(-c4ccc(-c5nnc(CC(=O)O)o5)cc4)cc3s2)cnn1C)c1ccccc1] |
| IT207 | 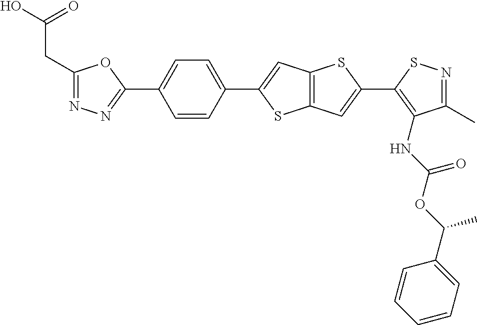 [SMILES: Cc1nsc(-c2cc3sc(-c4ccc(-c5nnc(CC(=O)O)o5)cc4)cc3s2)c1NC(=O)O[C@H](C)c1ccccc1] |
| IT208 | 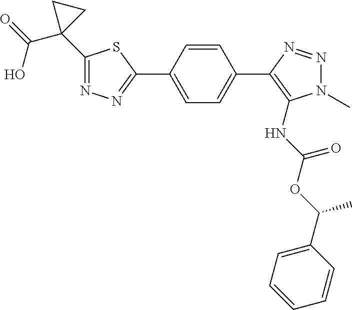 [SMILES: C[C@@H](OC(=O)Nc1c(-c2ccc(-c3nnc(C4(C(=O)O)CC4)s3)cc2)nnn1C)c1ccccc1] |
| IT209 | 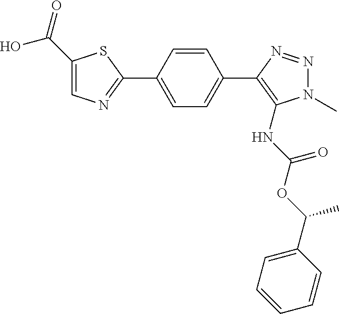 [SMILES: C[C@@H](OC(=O)Nc1c(-c2ccc(-c3ncc(C(=O)O)s3)cc2)nnn1C)c1ccccc1] |
| IT210 | 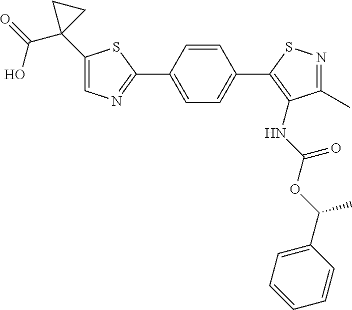 [SMILES: Cc1nsc(-c2ccc(-c3ncc(C4(C(=O)O)CC4)s3)cc2)c1NC(=O)O[C@H](C)c1ccccc1] |
| IT211 | 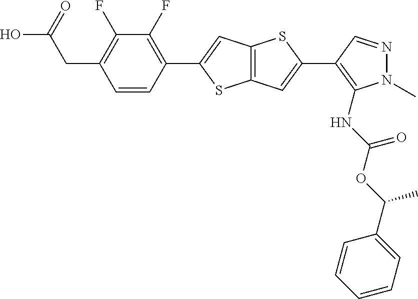 [SMILES: C[C@@H](OC(=O)Nc1c(-c2cc3sc(-c4ccc(CC(=O)O)c(F)c4F)cc3s2)cnn1C)c1ccccc1] |
| IT212 | 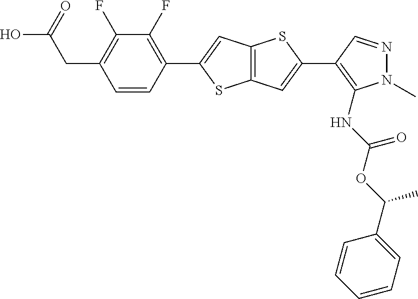 [SMILES: C[C@@H](OC(=O)Nc1c(-c2cc3sc(-c4ccc(CC(=O)O)c(F)c4F)cc3s2)cnn1C)c1ccccc1] |
| IT213 | 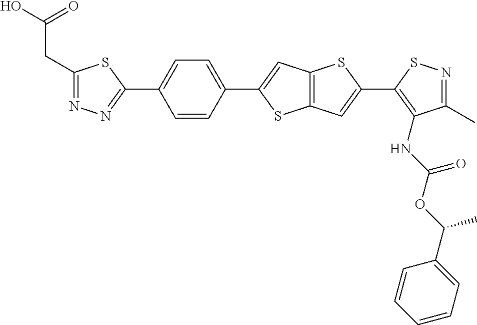 [SMILES: Cc1nsc(-c2cc3sc(-c4ccc(-c5nnc(CC(=O)O)s5)cc4)cc3s2)c1NC(=O)O[C@H](C)c1ccccc1] |
| IT214 | 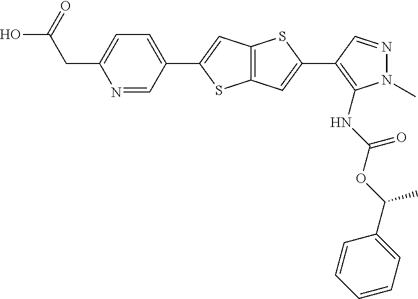 [SMILES: C[C@@H](OC(=O)Nc1c(-c2cc3sc(-c4ccc(CC(=O)O)nc4)cc3s2)cnn1C)c1ccccc1] |
| IT215 | 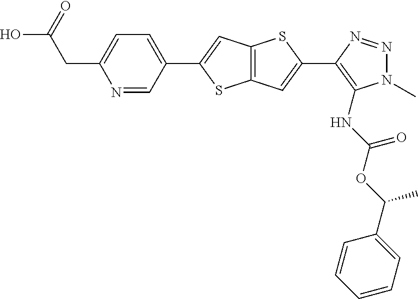 [SMILES: C[C@@H](OC(=O)Nc1c(-c2cc3sc(-c4ccc(CC(=O)O)nc4)cc3s2)nnn1C)c1ccccc1] |
| IT216 | 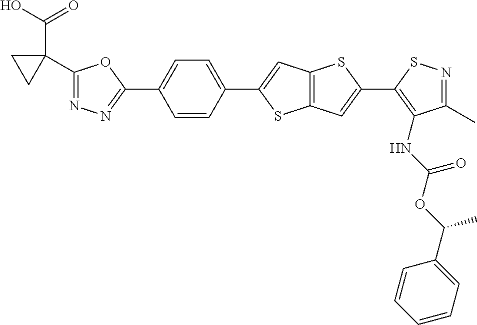 [SMILES: Cc1nsc(-c2cc3sc(-c4ccc(-c5nnc(C6(C(=O)O)CC6)o5)cc4)cc3s2)c1NC(=O)O[C@H](C)c1ccccc1] |
| IT217 | 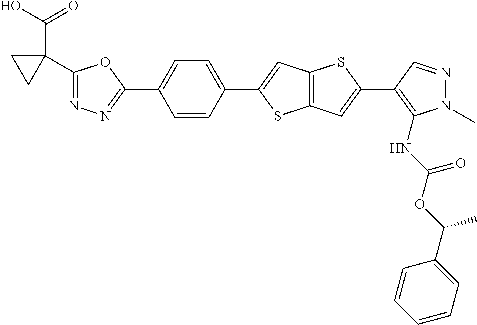 [SMILES: C[C@@H](OC(=O)Nc1c(-c2cc3sc(-c4ccc(-c5nnc(C6(C(=O)O)CC6)o5)cc4)cc3s2)cnn1C)c1ccccc1] |
| IT218 | 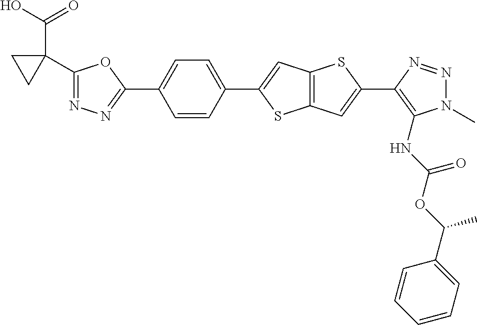 [SMILES: C[C@@H](OC(=O)Nc1c(-c2cc3sc(-c4ccc(-c5nnc(C6(C(=O)O)CC6)o5)cc4)cc3s2)nnn1C)c1ccccc1] |
| IT219 | 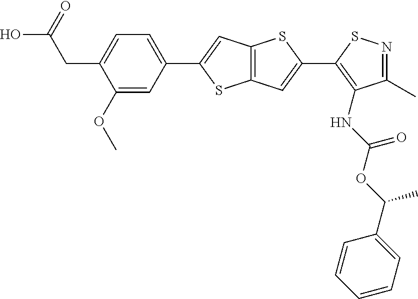 [SMILES: COc1cc(-c2cc3sc(-c4snc(C)c4NC(=O)O[C@H](C)c4ccccc4)cc3s2)ccc1CC(=O)O] |
| IT220 | 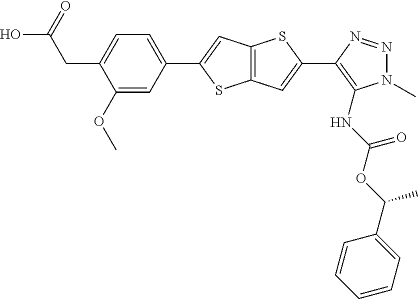 [SMILES: COc1cc(-c2cc3sc(-c4nnn(C)c4NC(=O)O[C@H](C)c4ccccc4)cc3s2)ccc1CC(=O)O] |
| IT221 | 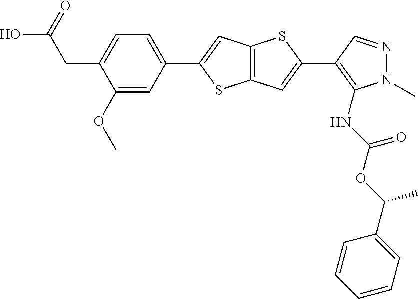 [SMILES: COc1cc(-c2cc3sc(-c4cnn(C)c4NC(=O)O[C@H](C)c4ccccc4)cc3s2)ccc1CC(=O)O] |
| IT222 | 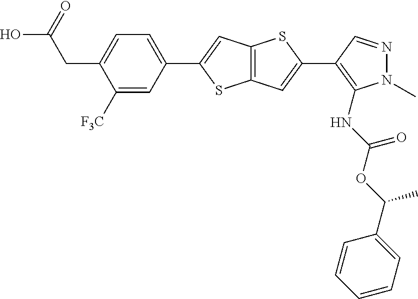 [SMILES: C[C@@H](OC(=O)Nc1c(-c2cc3sc(-c4ccc(CC(=O)O)c(C(F)(F)F)c4)cc3s2)cnn1C)c1ccccc1] |
| IT223 | 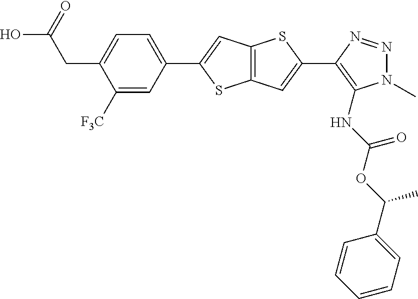 [SMILES: C[C@@H](OC(=O)Nc1c(-c2cc3sc(-c4ccc(CC(=O)O)c(C(F)(F)F)c4)cc3s2)nnn1C)c1ccccc1] |
| IT224 | 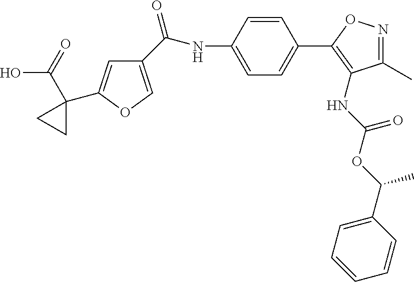 [SMILES: Cc1noc(-c2ccc(NC(=O)c3coc(C4(C(=O)O)CC4)c3)cc2)c1NC(=O)O[C@H](C)c1ccccc1] |
| IT225 | 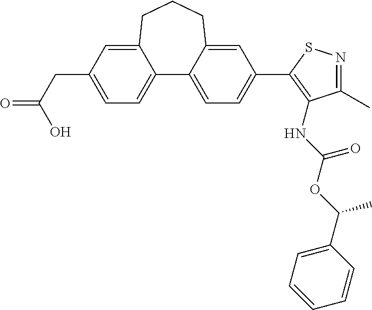 [SMILES: Cc1nsc(-c2ccc3c(c2)CCCc2cc(CC(=O)O)ccc2-3)c1NC(=O)O[C@H](C)c1ccccc1] |
| IT226 | 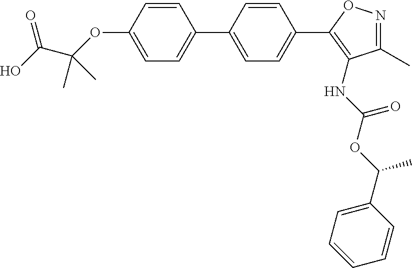 [SMILES: Cc1noc(-c2ccc(-c3ccc(OC(C)(C)C(=O)O)cc3)cc2)c1NC(=O)O[C@H](C)c1ccccc1] |
| IT227 | 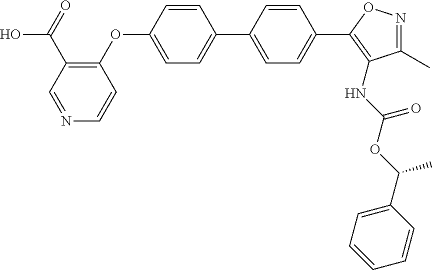 [SMILES: Cc1noc(-c2ccc(-c3ccc(Oc4ccncc4C(=O)O)cc3)cc2)c1NC(=O)O[C@H](C)c1ccccc1] |
| IT228 | 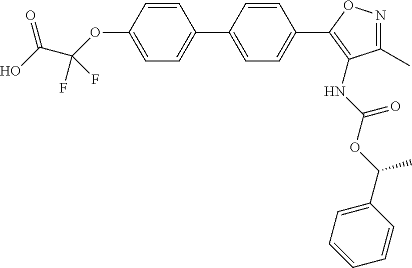 [SMILES: Cc1noc(-c2ccc(-c3ccc(OC(F)(F)C(=O)O)cc3)cc2)c1NC(=O)O[C@H](C)c1ccccc1] |
| IT229 | 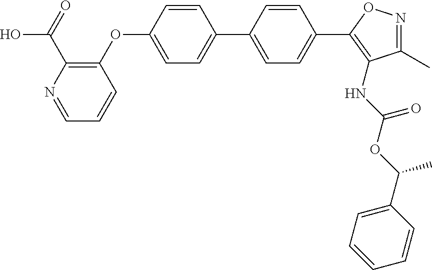 [SMILES: Cc1noc(-c2ccc(-c3ccc(Oc4cccnc4C(=O)O)cc3)cc2)c1NC(=O)O[C@H](C)c1ccccc1] |
| IT230 | 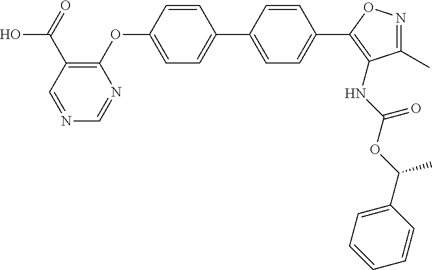 [SMILES: Cc1noc(-c2ccc(-c3ccc(Oc4ncncc4C(=O)O)cc3)cc2)c1NC(=O)O[C@H](C)c1ccccc1] |
| IT231 | 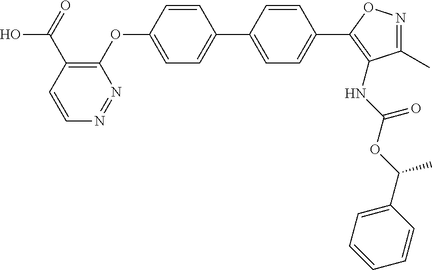 [SMILES: Cc1noc(-c2ccc(-c3ccc(Oc4nnccc4C(=O)O)cc3)cc2)c1NC(=O)O[C@H](C)c1ccccc1] |
| IT232 | 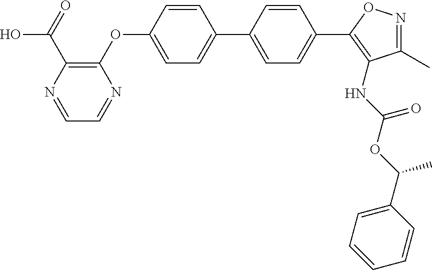 [SMILES: Cc1noc(-c2ccc(-c3ccc(Oc4nccnc4C(=O)O)cc3)cc2)c1NC(=O)O[C@H](C)c1ccccc1] |
| IT233 | 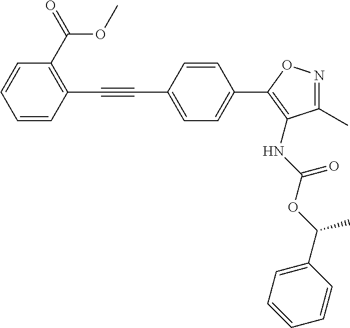 [SMILES: COC(=O)c1ccccc1C#Cc1ccc(-c2onc(C)c2NC(=O)O[C@H](C)c2ccccc2)cc1] |
| IT234 | 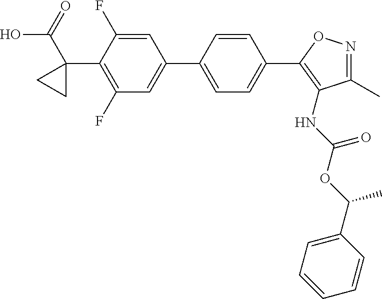 [SMILES: Cc1noc(-c2ccc(-c3cc(F)c(C4(C(=O)O)CC4)c(F)c3)cc2)c1NC(=O)O[C@H](C)c1ccccc1] |
| IT235 | 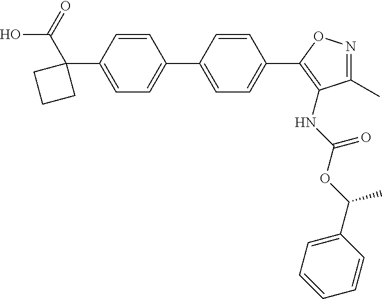 [SMILES: Cc1noc(-c2ccc(-c3ccc(C4(C(=O)O)CCC4)cc3)cc2)c1NC(=O)O[C@H](C)c1ccccc1] |
| IT236 | 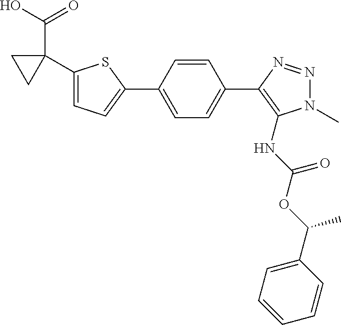 [SMILES: C[C@@H](OC(=O)Nc1c(-c2ccc(-c3ccc(C4(C(=O)O)CC4)s3)cc2)nnn1C)c1ccccc1] |
| IT237 | 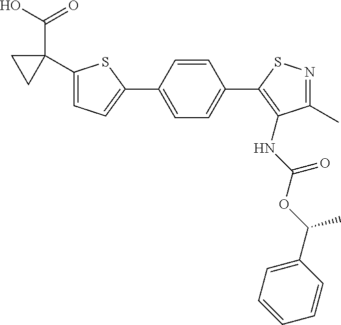 [SMILES: Cc1nsc(-c2ccc(-c3ccc(C4(C(=O)O)CC4)s3)cc2)c1NC(=O)O[C@H](C)c1ccccc1] |
| IT238 | 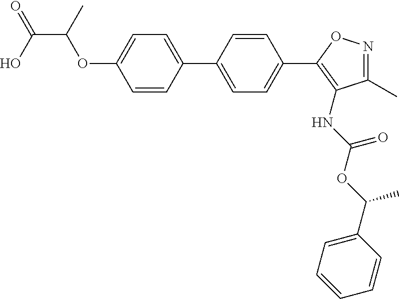 [SMILES: Cc1noc(-c2ccc(-c3ccc(OC(C)C(=O)O)cc3)cc2)c1NC(=O)O[C@H](C)c1ccccc1] |
| IT239 | 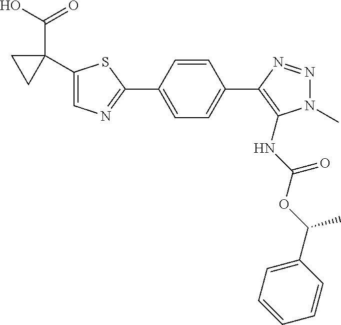 [SMILES: C[C@@H](OC(=O)Nc1c(-c2ccc(-c3ncc(C4(C(=O)O)CC4)s3)cc2)nnn1C)c1ccccc1] |
| IT240 | 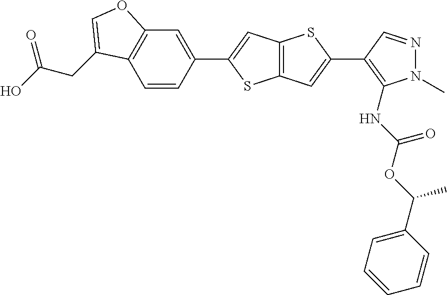 [SMILES: C[C@@H](OC(=O)Nc1c(-c2cc3sc(-c4ccc5c(CC(=O)O)coc5c4)cc3s2)cnn1C)c1ccccc1] |
| IT241 | 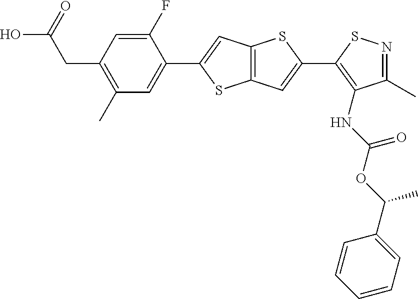 [SMILES: Cc1cc(-c2cc3sc(-c4snc(C)c4NC(=O)O[C@H](C)c4ccccc4)cc3s2)c(F)cc1CC(=O)O] |
| IT242 | 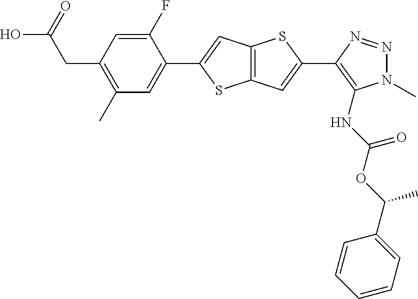 [SMILES: Cc1cc(-c2cc3sc(-c4nnn(C)c4NC(=O)O[C@H](C)c4ccccc4)cc3s2)c(F)cc1CC(=O)O] |
| IT243 | 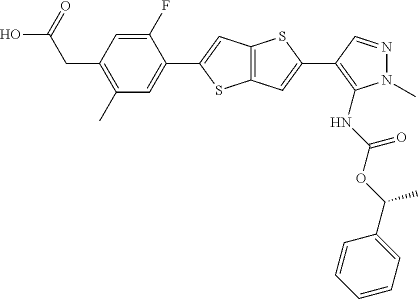 [SMILES: Cc1cc(-c2cc3sc(-c4cnn(C)c4NC(=O)O[C@H](C)c4ccccc4)cc3s2)c(F)cc1CC(=O)O] |
| IT244 | 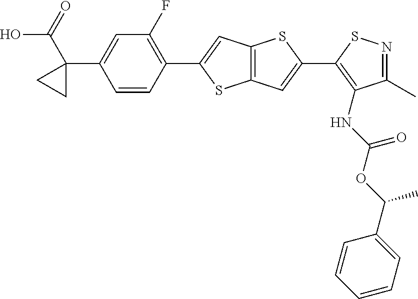 [SMILES: Cc1nsc(-c2cc3sc(-c4ccc(C5(C(=O)O)CC5)cc4F)cc3s2)c1NC(=O)O[C@H](C)c1ccccc1] |
| IT245 | 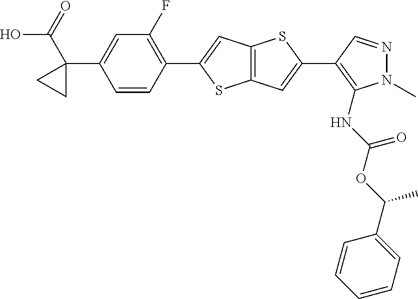 [SMILES: C[C@@H](OC(=O)Nc1c(-c2cc3sc(-c4ccc(C5(C(=O)O)CC5)cc4F)cc3s2)cnn1C)c1ccccc1] |
| IT246 | 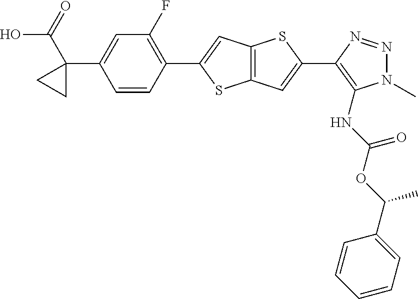 [SMILES: C[C@@H](OC(=O)Nc1c(-c2cc3sc(-c4ccc(C5(C(=O)O)CC5)cc4F)cc3s2)nnn1C)c1ccccc1] |
| IT247 | 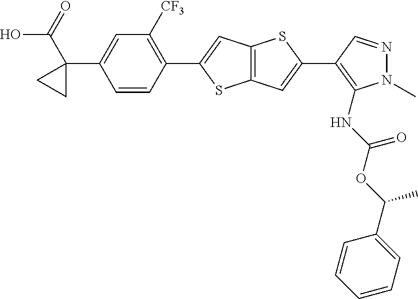 [SMILES: C[C@@H](OC(=O)Nc1c(-c2cc3sc(-c4ccc(C5(C(=O)O)CC5)cc4C(F)(F)F)cc3s2)cnn1C)c1ccccc1] |
| IT248 | 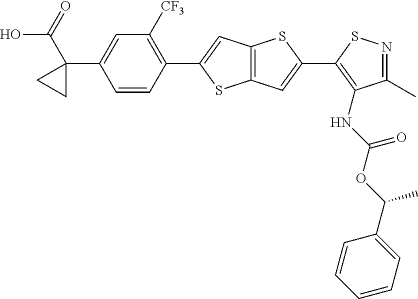 [SMILES: Cc1nsc(-c2cc3sc(-c4ccc(C5(C(=O)O)CC5)cc4C(F)(F)F)cc3s2)c1NC(=O)O[C@H](C)c1ccccc1] |
| IT249 | 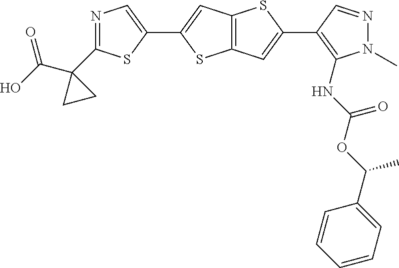 [SMILES: C[C@@H](OC(=O)Nc1c(-c2cc3sc(-c4cnc(C5(C(=O)O)CC5)s4)cc3s2)cnn1C)c1ccccc1] |
| IT250 | 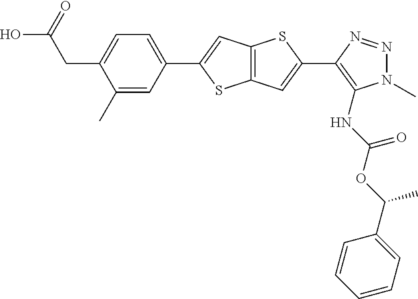 [SMILES: Cc1cc(-c2cc3sc(-c4nnn(C)c4NC(=O)O[C@H](C)c4ccccc4)cc3s2)ccc1CC(=O)O] |
| IT251 | 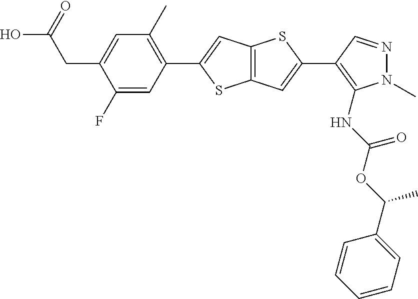 [SMILES: Cc1cc(CC(=O)O)c(F)cc1-c1cc2sc(-c3cnn(C)c3NC(=O)O[C@H](C)c3ccccc3)cc2s1] |
| IT252 | 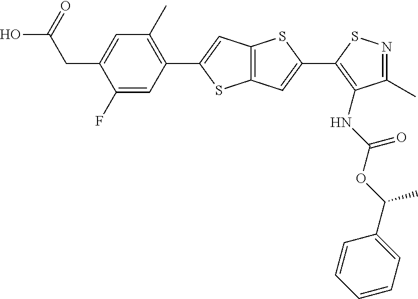 [SMILES: Cc1cc(CC(=O)O)c(F)cc1-c1cc2sc(-c3snc(C)c3NC(=O)O[C@H](C)c3ccccc3)cc2s1] |
| IT253 | 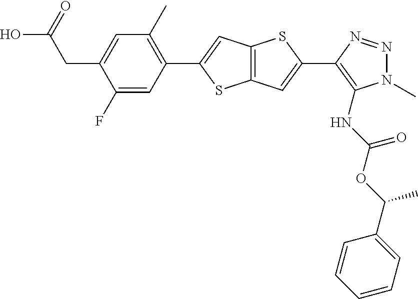 [SMILES: Cc1cc(CC(=O)O)c(F)cc1-c1cc2sc(-c3nnn(C)c3NC(=O)O[C@H](C)c3ccccc3)cc2s1] |
| IT254 | 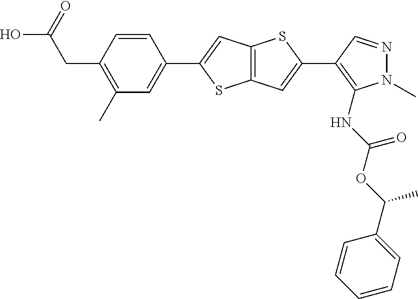 [SMILES: Cc1cc(-c2cc3sc(-c4cnn(C)c4NC(=O)O[C@H](C)c4ccccc4)cc3s2)ccc1CC(=O)O] |
| IT255 | 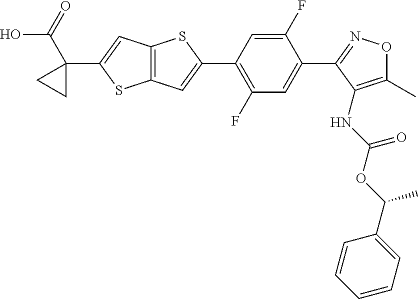 [SMILES: Cc1onc(-c2cc(F)c(-c3cc4sc(C5(C(=O)O)CC5)cc4s3)cc2F)c1NC(=O)O[C@H](C)c1ccccc1] |
| IT256 | 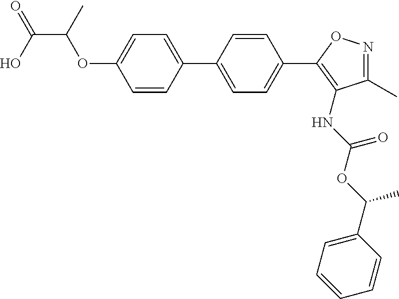 [SMILES: Cc1noc(-c2ccc(-c3ccc(OC(C)C(=O)O)cc3)cc2)c1NC(=O)O[C@H](C)c1ccccc1] |
| IT257 | 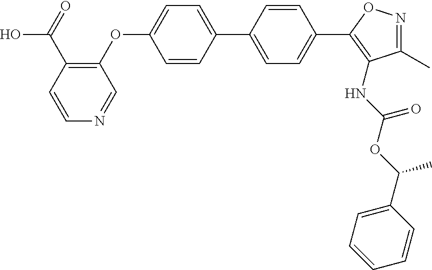 [SMILES: Cc1noc(-c2ccc(-c3ccc(Oc4cnccc4C(=O)O)cc3)cc2)c1NC(=O)O[C@H](C)c1ccccc1] |
| IT258 | 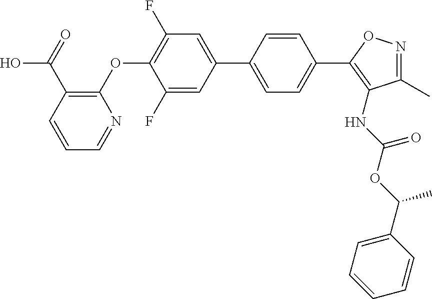 [SMILES: Cc1noc(-c2ccc(-c3cc(F)c(Oc4ncccc4C(=O)O)c(F)c3)cc2)c1NC(=O)O[C@H](C)c1ccccc1] |
| IT259 | 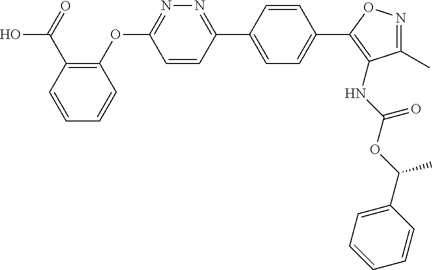 [SMILES: Cc1noc(-c2ccc(-c3ccc(Oc4ccccc4C(=O)O)nn3)cc2)c1NC(=O)O[C@H](C)c1ccccc1] |
| IT260 | 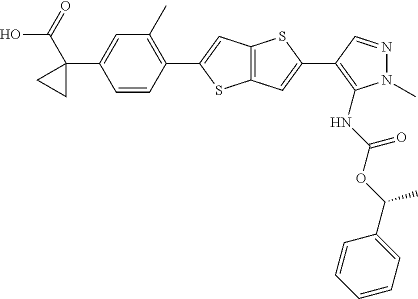 [SMILES: Cc1cc(C2(C(=O)O)CC2)ccc1-c1cc2sc(-c3cnn(C)c3NC(=O)O[C@H](C)c3ccccc3)cc2s1] |
| IT261 | 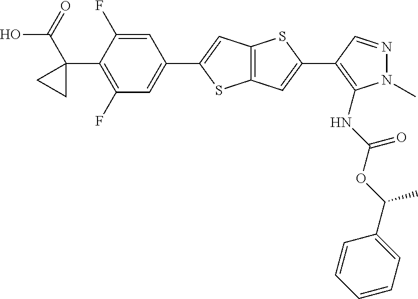 [SMILES: C[C@@H](OC(=O)Nc1c(-c2cc3sc(-c4cc(F)c(C5(C(=O)O)CC5)c(F)c4)cc3s2)cnn1C)c1ccccc1] |
| IT262 | 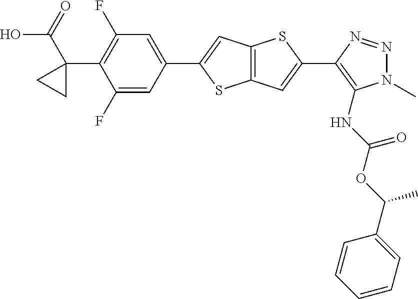 [SMILES: C[C@@H](OC(=O)Nc1c(-c2cc3sc(-c4cc(F)c(C5(C(=O)O)CC5)c(F)c4)cc3s2)nnn1C)c1ccccc1] |
| IT263 | 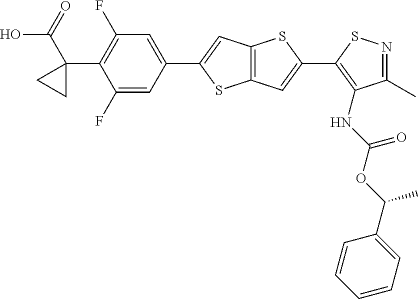 [SMILES: Cc1nsc(-c2cc3sc(-c4cc(F)c(C5(C(=O)O)CC5)c(F)c4)cc3s2)c1NC(=O)O[C@H](C)c1ccccc1] |
| IT264 | 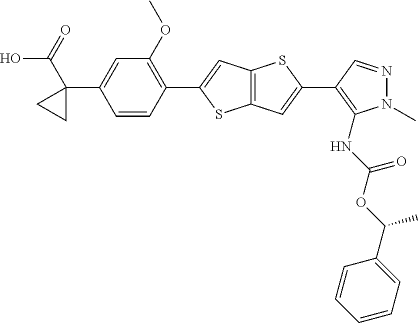 [SMILES: COc1cc(C2(C(=O)O)CC2)ccc1-c1cc2sc(-c3cnn(C)c3NC(=O)O[C@H](C)c3ccccc3)cc2s1] |
| IT265 | 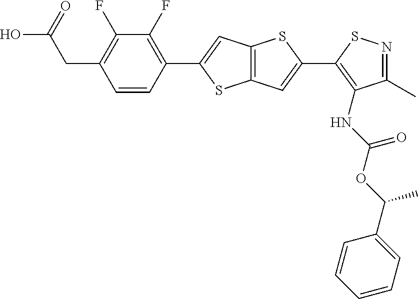 [SMILES: Cc1nsc(-c2cc3sc(-c4ccc(CC(=O)O)c(F)c4F)cc3s2)c1NC(=O)O[C@H](C)c1ccccc1] |
| IT266 | 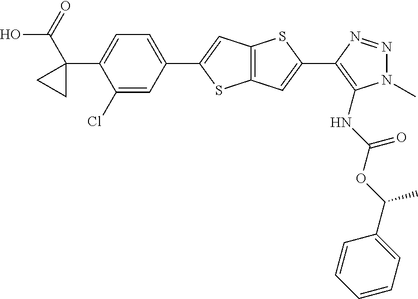 [SMILES: C[C@@H](OC(=O)Nc1c(-c2cc3sc(-c4ccc(C5(C(=O)O)CC5)c(Cl)c4)cc3s2)nnn1C)c1ccccc1] |
| IT267 | 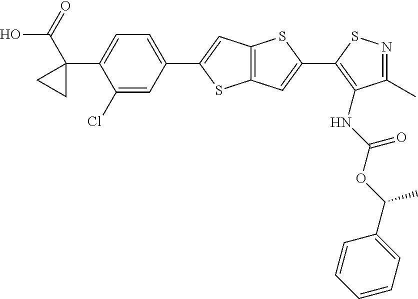 [SMILES: Cc1nsc(-c2cc3sc(-c4ccc(C5(C(=O)O)CC5)c(Cl)c4)cc3s2)c1NC(=O)O[C@H](C)c1ccccc1] |
| IT268 | 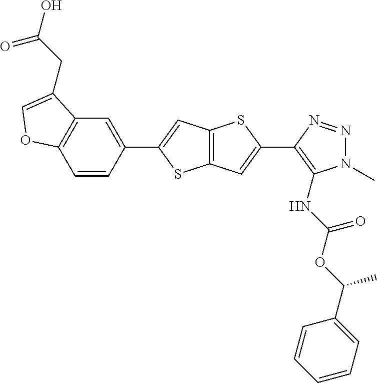 [SMILES: C[C@@H](OC(=O)Nc1c(-c2cc3sc(-c4ccc5occ(CC(=O)O)c5c4)cc3s2)nnn1C)c1ccccc1] |
| IT269 | 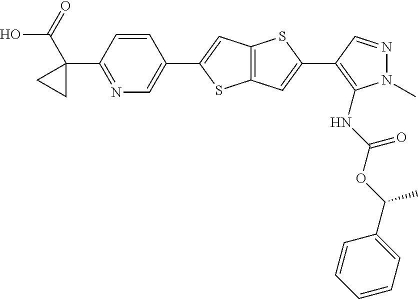 [SMILES: C[C@@H](OC(=O)Nc1c(-c2cc3sc(-c4ccc(C5(C(=O)O)CC5)nc4)cc3s2)cnn1C)c1ccccc1] |
| IT270 | 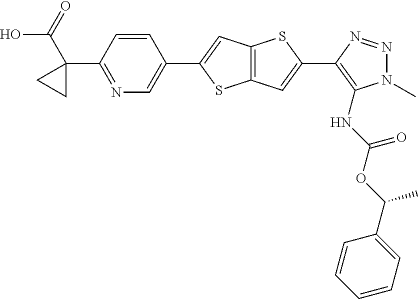 [SMILES: C[C@@H](OC(=O)Nc1c(-c2cc3sc(-c4ccc(C5(C(=O)O)CC5)nc4)cc3s2)nnn1C)c1ccccc1] |
| IT271 | 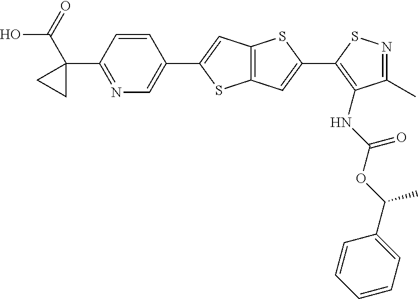 [SMILES: Cc1nsc(-c2cc3sc(-c4ccc(C5(C(=O)O)CC5)nc4)cc3s2)c1NC(=O)O[C@H](C)c1ccccc1] |
| IT272 | 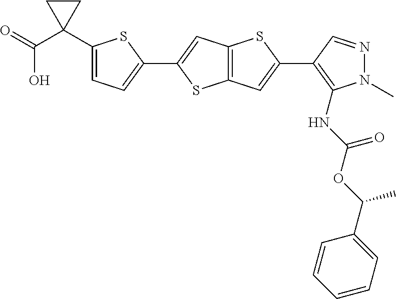 [SMILES: C[C@@H](OC(=O)Nc1c(-c2cc3sc(-c4ccc(C5(C(=O)O)CC5)s4)cc3s2)cnn1C)c1ccccc1] |
| IT273 | 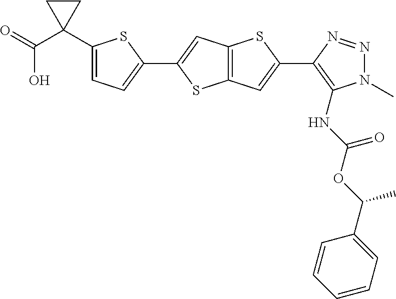 [SMILES: C[C@@H](OC(=O)Nc1c(-c2cc3sc(-c4ccc(C5(C(=O)O)CC5)s4)cc3s2)nnn1C)c1ccccc1] |
| IT274 | 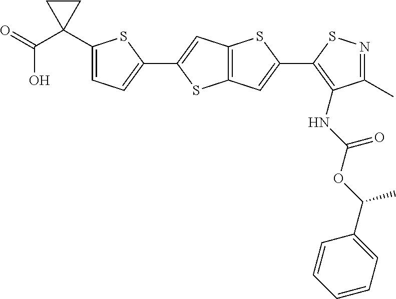 [SMILES: Cc1nsc(-c2cc3sc(-c4ccc(C5(C(=O)O)CC5)s4)cc3s2)c1NC(=O)O[C@H](C)c1ccccc1] |
| IT275 | 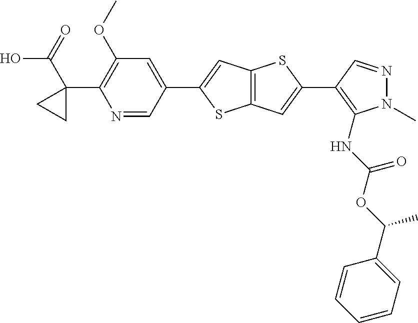 [SMILES: COc1cc(-c2cc3sc(-c4cnn(C)c4NC(=O)O[C@H](C)c4ccccc4)cc3s2)cnc1C1(C(=O)O)CC1] |
| IT276 | 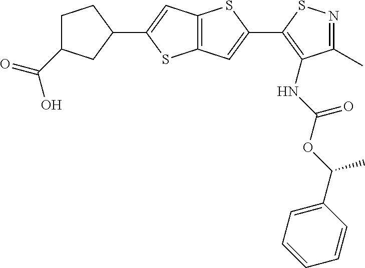 [SMILES: Cc1nsc(-c2cc3sc(C4CCC(C(=O)O)C4)cc3s2)c1NC(=O)O[C@H](C)c1ccccc1] |
| IT277 | 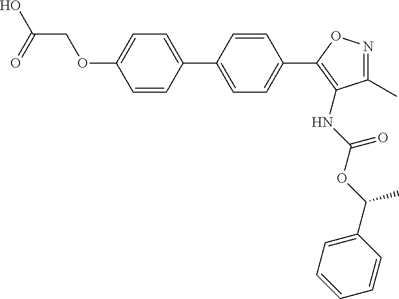 [SMILES: Cc1noc(-c2ccc(-c3ccc(OCC(=O)O)cc3)cc2)c1NC(=O)O[C@H](C)c1ccccc1] |
| IT278 | 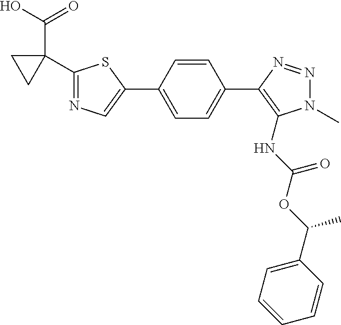 [SMILES: C[C@@H](OC(=O)Nc1c(-c2ccc(-c3cnc(C4(C(=O)O)CC4)s3)cc2)nnn1C)c1ccccc1] |
| IT279 | 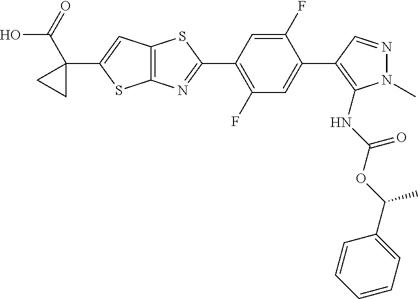 [SMILES: C[C@@H](OC(=O)Nc1c(-c2cc(F)c(-c3nc4sc(C5(C(=O)O)CC5)cc4s3)cc2F)cnn1C)c1ccccc1] |
| IT280 | 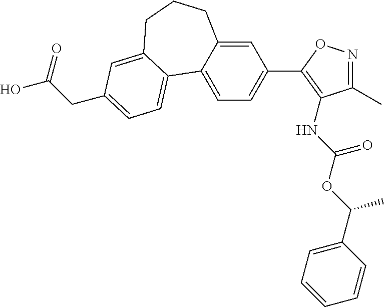 [SMILES: Cc1noc(-c2ccc3c(c2)CCCc2cc(CC(=O)O)ccc2-3)c1NC(=O)O[C@H](C)c1ccccc1] |
| IT281 | 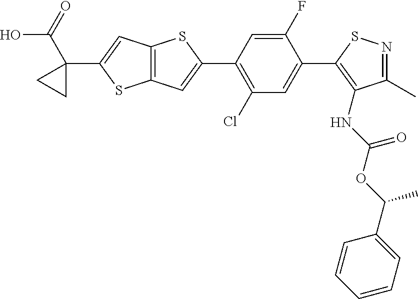 [SMILES: Cc1nsc(-c2cc(Cl)c(-c3cc4sc(C5(C(=O)O)CC5)cc4s3)cc2F)c1NC(=O)O[C@H](C)c1ccccc1] |
| IT282 | 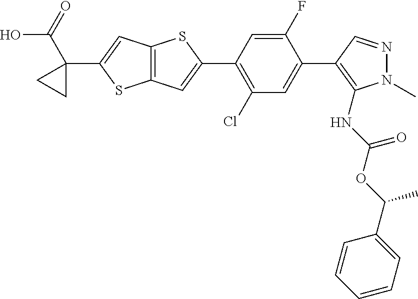 [SMILES: C[C@@H](OC(=O)Nc1c(-c2cc(Cl)c(-c3cc4sc(C5(C(=O)O)CC5)cc4s3)cc2F)cnn1C)c1ccccc1] |
| IT283 | 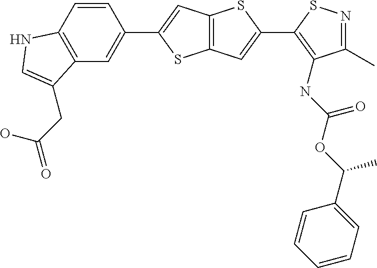 [SMILES: Cc1nsc(-c2cc3sc(-c4ccc5[nH]cc(CC(=O)O)c5c4)cc3s2)c1NC(=O)O[C@H](C)c1ccccc1] |
| IT284 | 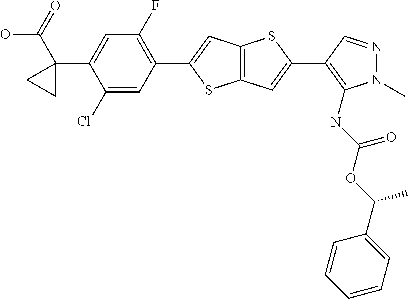 [SMILES: C[C@@H](OC(=O)Nc1c(-c2cc3sc(-c4cc(Cl)c(C5(C(=O)O)CC5)cc4F)cc3s2)cnn1C)c1ccccc1] |
| IT285 | 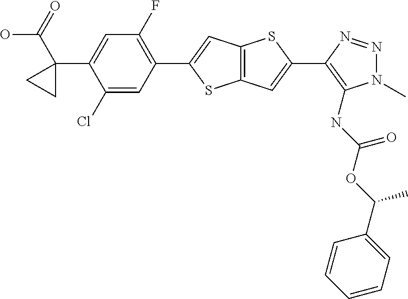 [SMILES: C[C@@H](OC(=O)Nc1c(-c2cc3sc(-c4cc(Cl)c(C5(C(=O)O)CC5)cc4F)cc3s2)nnn1C)c1ccccc1] |
| IT286 | 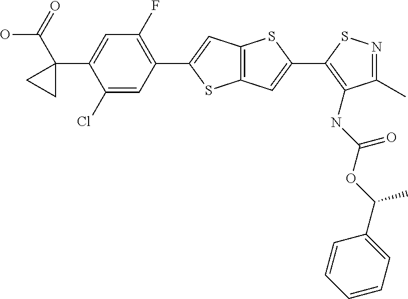 [SMILES: Cc1nsc(-c2cc3sc(-c4cc(Cl)c(C5(C(=O)O)CC5)cc4F)cc3s2)c1NC(=O)O[C@H](C)c1ccccc1] |
| IT287 | 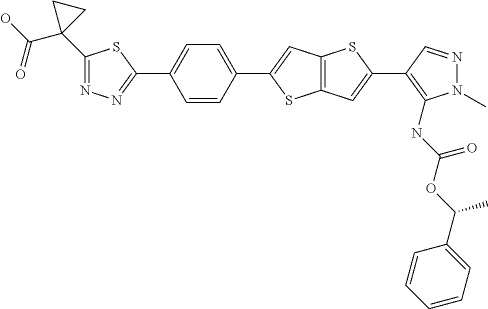 [SMILES: C[C@@H](OC(=O)Nc1c(-c2cc3sc(-c4ccc(-c5nnc(C6(C(=O)O)CC6)s5)cc4)cc3s2)cnn1C)c1ccccc1] |
| IT288 | 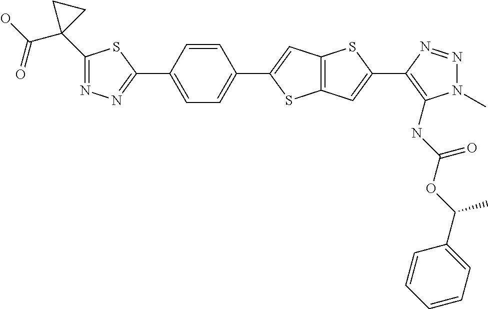 [SMILES: C[C@@H](OC(=O)Nc1c(-c2cc3sc(-c4ccc(-c5nnc(C6(C(=O)O)CC6)s5)cc4)cc3s2)nnn1C)c1ccccc1] |
| IT289 | 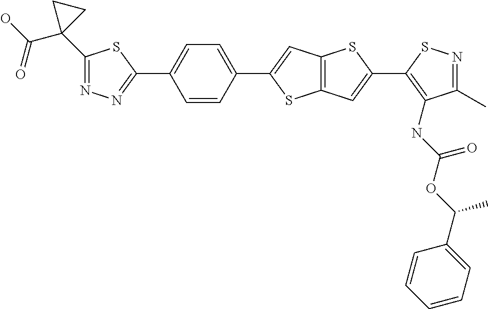 [SMILES: Cc1nsc(-c2cc3sc(-c4ccc(-c5nnc(C6(C(=O)O)CC6)s5)cc4)cc3s2)c1NC(=O)O[C@H](C)c1ccccc1] |
| IT290 | 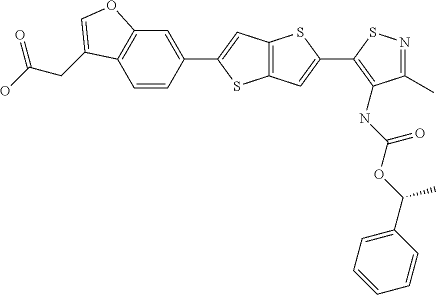 [SMILES: Cc1nsc(-c2cc3sc(-c4ccc5c(CC(=O)O)coc5c4)cc3s2)c1NC(=O)O[C@H](C)c1ccccc1] |
| IT291 | 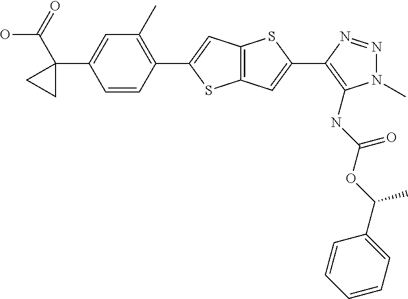 [SMILES: Cc1cc(C2(C(=O)O)CC2)ccc1-c1cc2sc(-c3nnn(C)c3NC(=O)O[C@H](C)c3ccccc3)cc2s1] |
| IT292 | 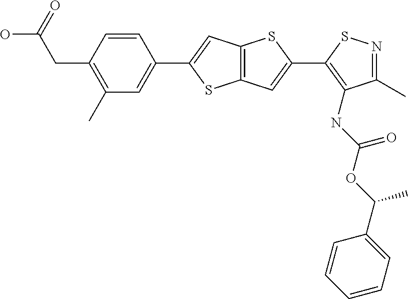 [SMILES: Cc1cc(-c2cc3sc(-c4snc(C)c4NC(=O)O[C@H](C)c4ccccc4)cc3s2)ccc1CC(=O)O] |
| IT293 | 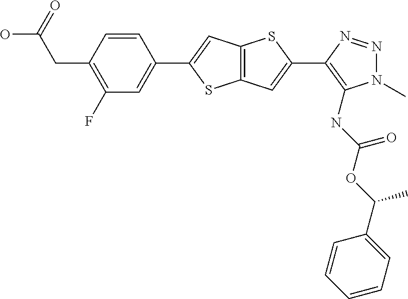 [SMILES: C[C@@H](OC(=O)Nc1c(-c2cc3sc(-c4ccc(CC(=O)O)c(F)c4)cc3s2)nnn1C)c1ccccc1] |
| IT294 | 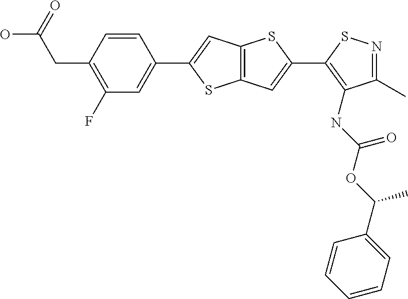 [SMILES: Cc1nsc(-c2cc3sc(-c4ccc(CC(=O)O)c(F)c4)cc3s2)c1NC(=O)O[C@H](C)c1ccccc1] |
| IT295 | 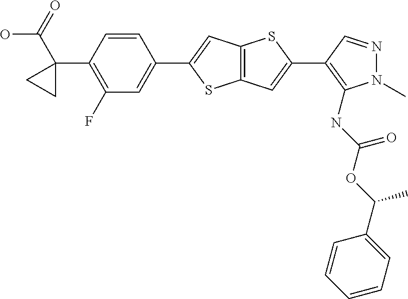 [SMILES: C[C@@H](OC(=O)Nc1c(-c2cc3sc(-c4ccc(C5(C(=O)O)CC5)c(F)c4)cc3s2)cnn1C)c1ccccc1] |
| IT296 | 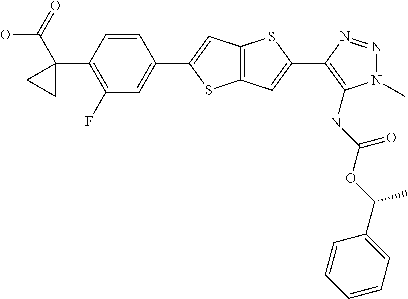 [SMILES: C[C@@H](OC(=O)Nc1c(-c2cc3sc(-c4ccc(C5(C(=O)O)CC5)c(F)c4)cc3s2)nnn1C)c1ccccc1] |
| IT297 | 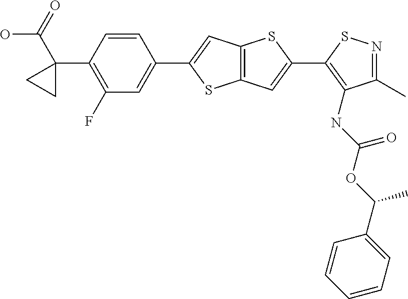 [SMILES: Cc1nsc(-c2cc3sc(-c4ccc(C5(C(=O)O)CC5)c(F)c4)cc3s2)c1NC(=O)O[C@H](C)c1ccccc1] |
| IT298 | 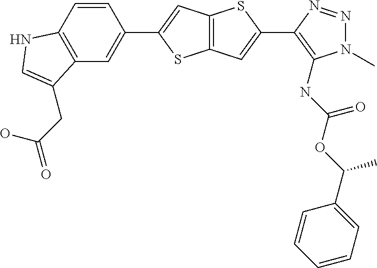 [SMILES: C[C@@H](OC(=O)Nc1c(-c2cc3sc(-c4ccc5[nH]cc(CC(=O)O)c5c4)cc3s2)nnn1C)c1ccccc1] |
| IT299 | 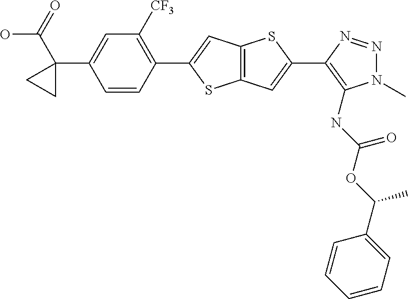 [SMILES: C[C@@H](OC(=O)Nc1c(-c2cc3sc(-c4ccc(C5(C(=O)O)CC5)cc4C(F)(F)F)cc3s2)nnn1C)c1ccccc1] |
| IT300 | 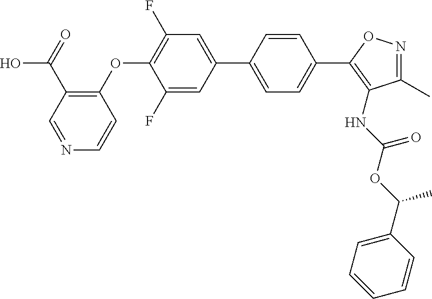 [SMILES: Cc1noc(-c2ccc(-c3cc(F)c(Oc4ccncc4C(=O)O)c(F)c3)cc2)c1NC(=O)O[C@H](C)c1ccccc1] |
| IT301 | 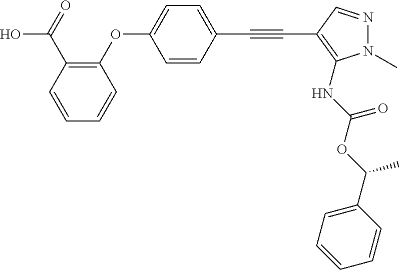 [SMILES: C[C@@H](OC(=O)Nc1c(C#Cc2ccc(Oc3ccccc3C(=O)O)cc2)cnn1C)c1ccccc1] |
| IT302 | 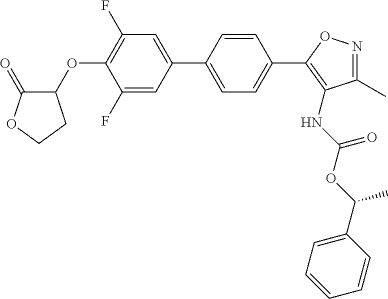 [SMILES: Cc1noc(-c2ccc(-c3cc(F)c(OC4CCOC4=O)c(F)c3)cc2)c1NC(=O)O[C@H](C)c1ccccc1] |
| IT303 | 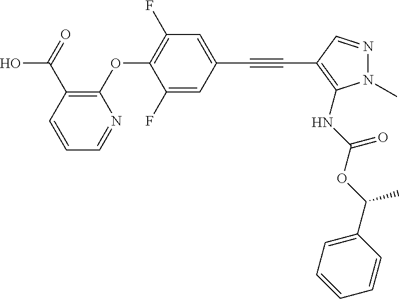 [SMILES: C[C@@H](OC(=O)Nc1c(C#Cc2cc(F)c(Oc3ncccc3C(=O)O)c(F)c2)cnn1C)c1ccccc1] |
| IT304 | 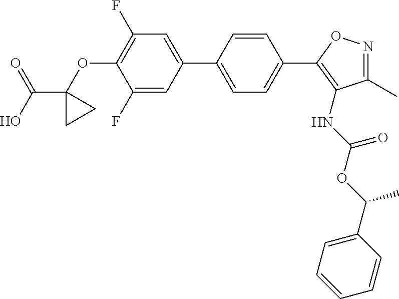 [SMILES: Cc1noc(-c2ccc(-c3cc(F)c(OC4(C(=O)O)CC4)c(F)c3)cc2)c1NC(=O)O[C@H](C)c1ccccc1] |
| IT305 | 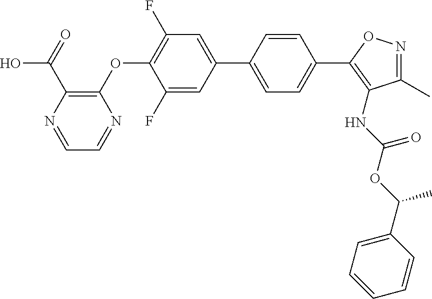 [SMILES: Cc1noc(-c2ccc(-c3cc(F)c(Oc4nccnc4C(=O)O)c(F)c3)cc2)c1NC(=O)O[C@H](C)c1ccccc1] |
| IT306 | 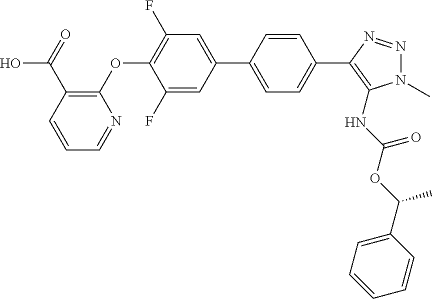 [SMILES: C[C@@H](OC(=O)Nc1c(-c2ccc(-c3cc(F)c(Oc4ncccc4C(=O)O)c(F)c3)cc2)nnn1C)c1ccccc1] |
| IT307 | 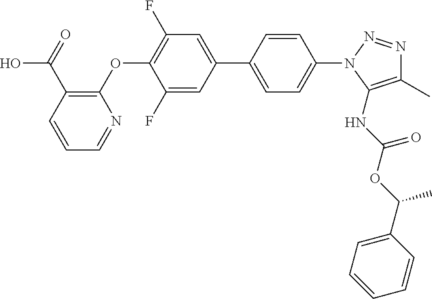 [SMILES: Cc1nnn(-c2ccc(-c3cc(F)c(Oc4ncccc4C(=O)O)c(F)c3)cc2)c1NC(=O)O[C@H](C)c1ccccc1] |
| IT308 | 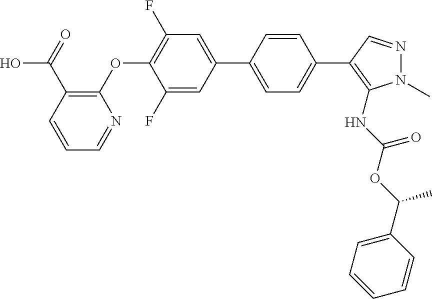 [SMILES: C[C@@H](OC(=O)Nc1c(-c2ccc(-c3cc(F)c(Oc4ncccc4C(=O)O)c(F)c3)cc2)cnn1C)c1ccccc1] |
| IT309 | 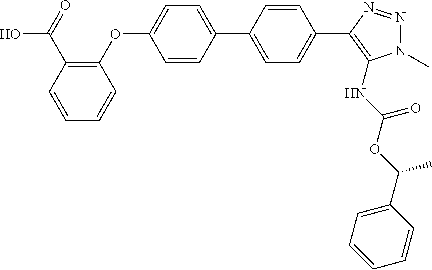 [SMILES: C[C@@H](OC(=O)Nc1c(-c2ccc(-c3ccc(Oc4ccccc4C(=O)O)cc3)cc2)nnn1C)c1ccccc1] |
| IT310 | 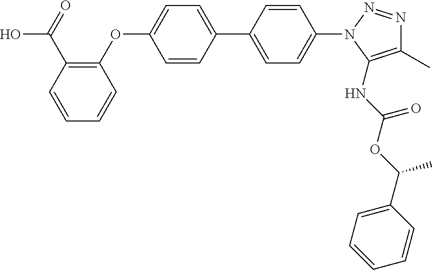 [SMILES: Cc1nnn(-c2ccc(-c3ccc(Oc4ccccc4C(=O)O)cc3)cc2)c1NC(=O)O[C@H](C)c1ccccc1] |
| IT311 | 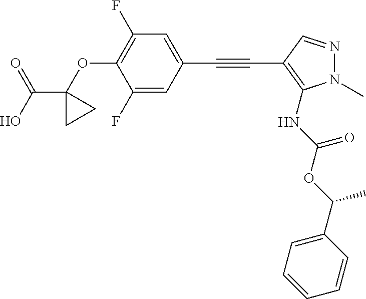 [SMILES: C[C@@H](OC(=O)Nc1c(C#Cc2cc(F)c(OC3(C(=O)O)CC3)c(F)c2)cnn1C)c1ccccc1] |
| IT312 | 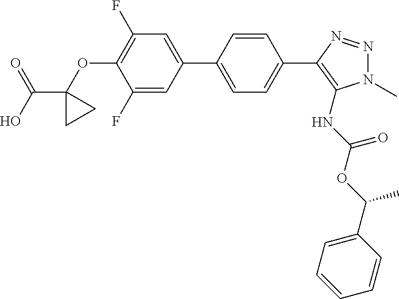 [SMILES: C[C@@H](OC(=O)Nc1c(-c2ccc(-c3cc(F)c(OC4(C(=O)O)CC4)c(F)c3)cc2)nnn1C)c1ccccc1] |
| IT313 | 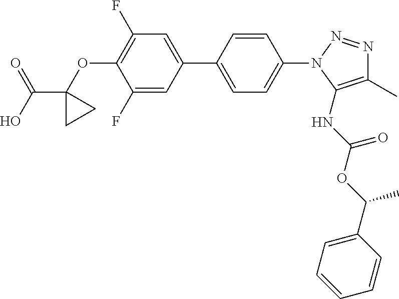 [SMILES: Cc1nnn(-c2ccc(-c3cc(F)c(OC4(C(=O)O)CC4)c(F)c3)cc2)c1NC(=O)O[C@H](C)c1ccccc1] |
| IT314 | 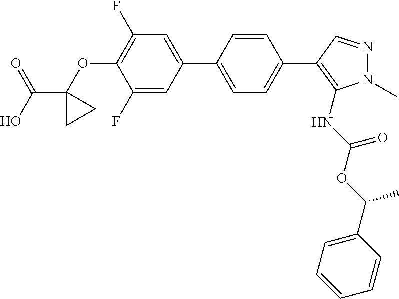 [SMILES: C[C@@H](OC(=O)Nc1c(-c2ccc(-c3cc(F)c(OC4(C(=O)O)CC4)c(F)c3)cc2)cnn1C)c1ccccc1] |
| IT315 | 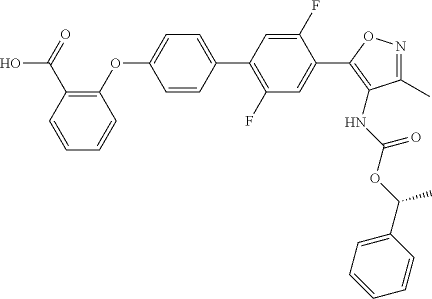 [SMILES: Cc1noc(-c2cc(F)c(-c3ccc(Oc4ccccc4C(=O)O)cc3)cc2F)c1NC(=O)O[C@H](C)c1ccccc1] |
| IT316 | 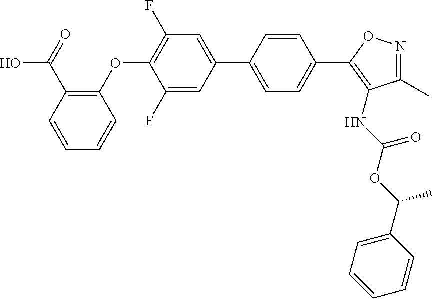 [SMILES: Cc1noc(-c2ccc(-c3cc(F)c(Oc4ccccc4C(=O)O)c(F)c3)cc2)c1NC(=O)O[C@H](C)c1ccccc1] |
| IT317 | 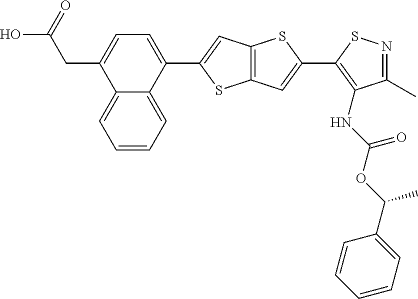 [SMILES: Cc1nsc(-c2cc3sc(-c4ccc(CC(=O)O)c5ccccc45)cc3s2)c1NC(=O)O[C@H](C)c1ccccc1] |
| IT318 | 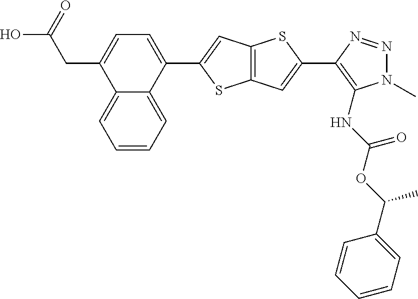 [SMILES: C[C@@H](OC(=O)Nc1c(-c2cc3sc(-c4ccc(CC(=O)O)c5ccccc45)cc3s2)nnn1C)c1ccccc1] |
| IT319 | 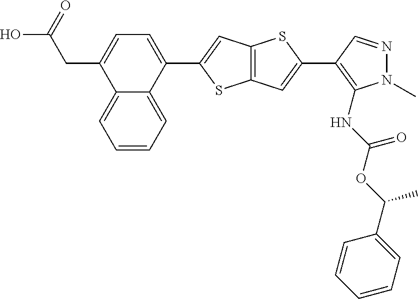 [SMILES: C[C@@H](OC(=O)Nc1c(-c2cc3sc(-c4ccc(CC(=O)O)c5ccccc45)cc3s2)cnn1C)c1ccccc1] |
| IT320 | 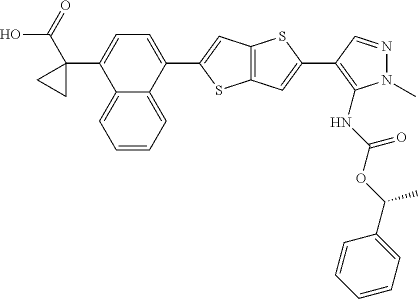 [SMILES: C[C@@H](OC(=O)Nc1c(-c2cc3sc(-c4ccc(C5(C(=O)O)CC5)c5ccccc45)cc3s2)cnn1C)c1ccccc1] |
| IT321 | 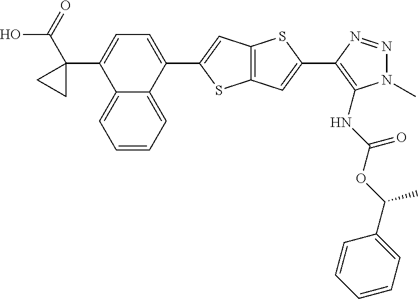 [SMILES: C[C@@H](OC(=O)Nc1c(-c2cc3sc(-c4ccc(C5(C(=O)O)CC5)c5ccccc45)cc3s2)nnn1C)c1ccccc1] |
| IT322 | 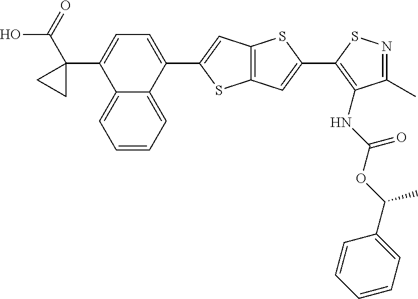 [SMILES: Cc1nsc(-c2cc3sc(-c4ccc(C5(C(=O)O)CC5)c5ccccc45)cc3s2)c1NC(=O)O[C@H](C)c1ccccc1] |
| IT323 | 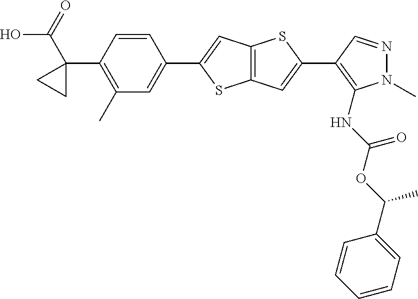 [SMILES: Cc1cc(-c2cc3sc(-c4cnn(C)c4NC(=O)O[C@H](C)c4ccccc4)cc3s2)ccc1C1(C(=O)O)CC1] |
| IT324 | 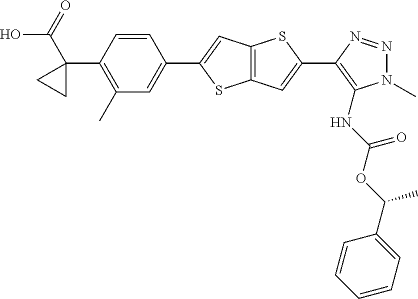 [SMILES: Cc1cc(-c2cc3sc(-c4nnn(C)c4NC(=O)O[C@H](C)c4ccccc4)cc3s2)ccc1C1(C(=O)O)CC1] |
| IT325 | 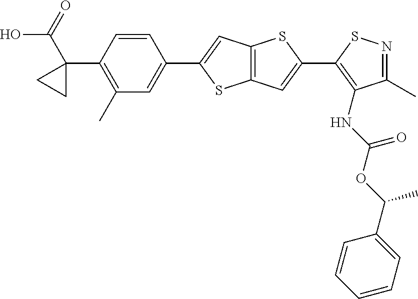 [SMILES: Cc1cc(-c2cc3sc(-c4snc(C)c4NC(=O)O[C@H](C)c4ccccc4)cc3s2)ccc1C1(C(=O)O)CC1] |
| IT326 | 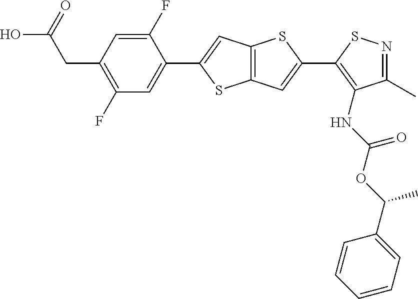 [SMILES: Cc1nsc(-c2cc3sc(-c4cc(F)c(CC(=O)O)cc4F)cc3s2)c1NC(=O)O[C@H](C)c1ccccc1] |
| IT327 | 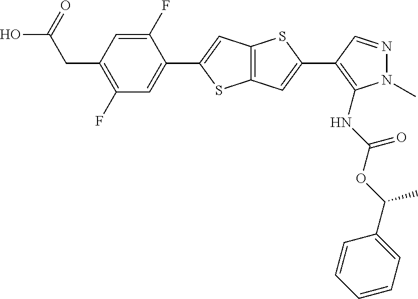 [SMILES: C[C@@H](OC(=O)Nc1c(-c2cc3sc(-c4cc(F)c(CC(=O)O)cc4F)cc3s2)cnn1C)c1ccccc1] |
| IT328 | 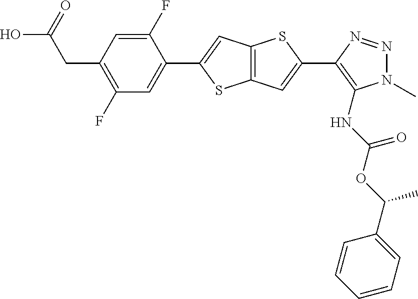 [SMILES: C[C@@H](OC(=O)Nc1c(-c2cc3sc(-c4cc(F)c(CC(=O)O)cc4F)cc3s2)nnn1C)c1ccccc1] |
| IT329 | 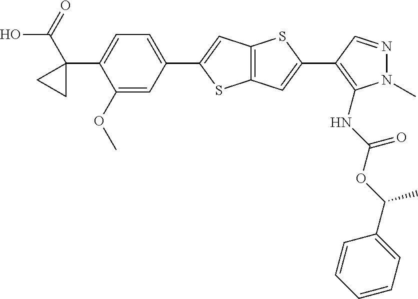 [SMILES: COc1cc(-c2cc3sc(-c4cnn(C)c4NC(=O)O[C@H](C)c4ccccc4)cc3s2)ccc1C1(C(=O)O)CC1] |
| IT330 | 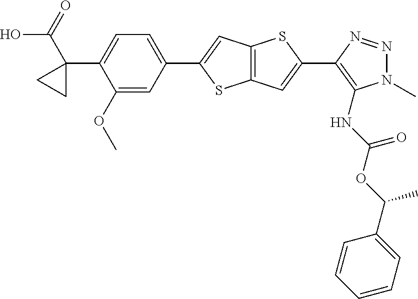 [SMILES: COc1cc(-c2cc3sc(-c4nnn(C)c4NC(=O)O[C@H](C)c4ccccc4)cc3s2)ccc1C1(C(=O)O)CC1] |
| IT331 | 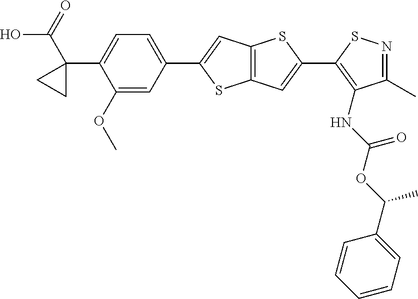 [SMILES: COc1cc(-c2cc3sc(-c4snc(C)c4NC(=O)O[C@H](C)c4ccccc4)cc3s2)ccc1C1(C(=O)O)CC1] |
| IT332 | 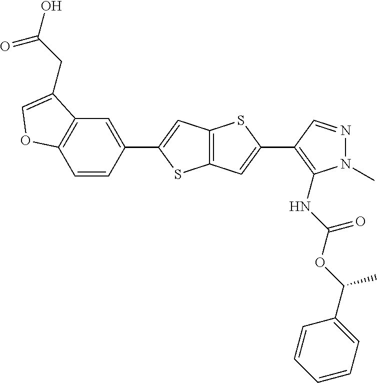 [SMILES: C[C@@H](OC(=O)Nc1c(-c2cc3sc(-c4ccc5occ(CC(=O)O)c5c4)cc3s2)cnn1C)c1ccccc1] |
| IT333 | 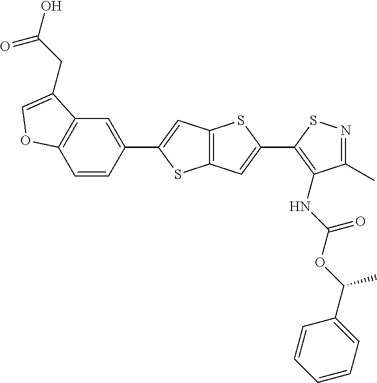 [SMILES: Cc1nsc(-c2cc3sc(-c4ccc5occ(CC(=O)O)c5c4)cc3s2)c1NC(=O)O[C@H](C)c1ccccc1] |
| IT334 | 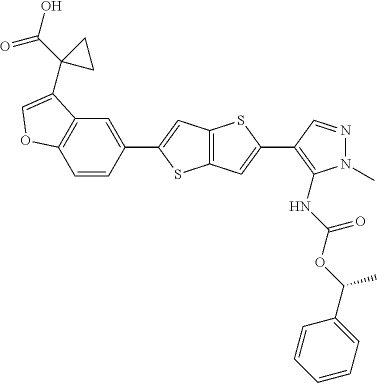 [SMILES: C[C@@H](OC(=O)Nc1c(-c2cc3sc(-c4ccc5occ(C6(C(=O)O)CC6)c5c4)cc3s2)cnn1C)c1ccccc1] |
| IT335 | 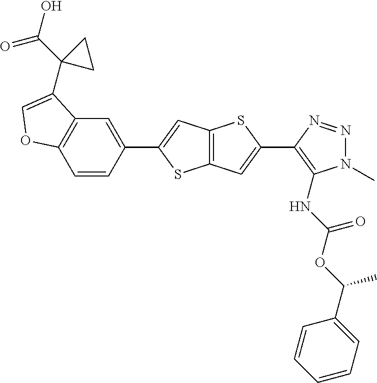 [SMILES: C[C@@H](OC(=O)Nc1c(-c2cc3sc(-c4ccc5occ(C6(C(=O)O)CC6)c5c4)cc3s2)nnn1C)c1ccccc1] |
| IT336 | 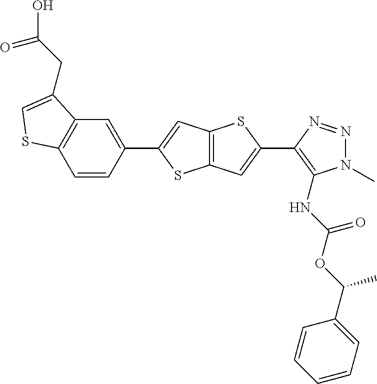 [SMILES: C[C@@H](OC(=O)Nc1c(-c2cc3sc(-c4ccc5scc(CC(=O)O)c5c4)cc3s2)nnn1C)c1ccccc1] |
| IT337 | 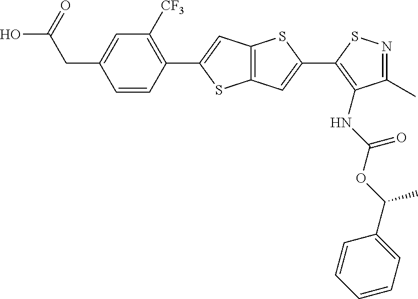 [SMILES: Cc1nsc(-c2cc3sc(-c4ccc(CC(=O)O)cc4C(F)(F)F)cc3s2)c1NC(=O)O[C@H](C)c1ccccc1] |
| IT338 | 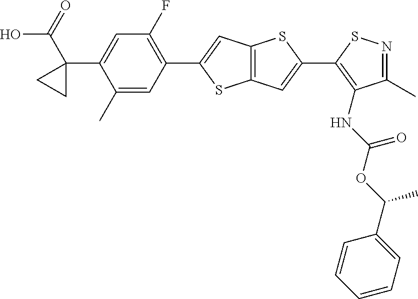 [SMILES: Cc1cc(-c2cc3sc(-c4snc(C)c4NC(=O)O[C@H](C)c4ccccc4)cc3s2)c(F)cc1C1(C(=O)O)CC1] |
| IT339 | 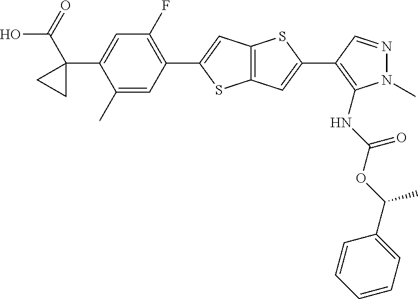 [SMILES: Cc1cc(-c2cc3sc(-c4cnn(C)c4NC(=O)O[C@H](C)c4ccccc4)cc3s2)c(F)cc1C1(C(=O)O)CC1] |
| IT340 | 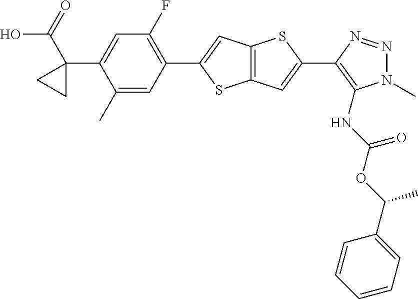 [SMILES: Cc1cc(-c2cc3sc(-c4nnn(C)c4NC(=O)O[C@H](C)c4ccccc4)cc3s2)c(F)cc1C1(C(=O)O)CC1] |
| IT341 | 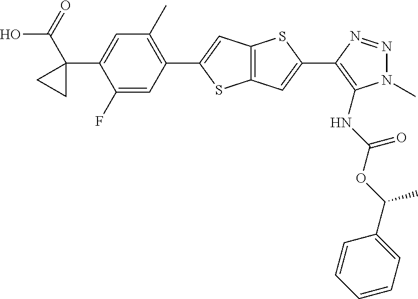 [SMILES: Cc1cc(C2(C(=O)O)CC2)c(F)cc1-c1cc2sc(-c3nnn(C)c3NC(=O)O[C@H](C)c3ccccc3)cc2s1] |
| IT342 | 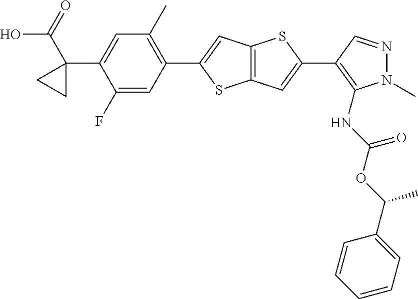 [SMILES: Cc1cc(C2(C(=O)O)CC2)c(F)cc1-c1cc2sc(-c3cnn(C)c3NC(=O)O[C@H](C)c3ccccc3)cc2s1] |
| IT343 | 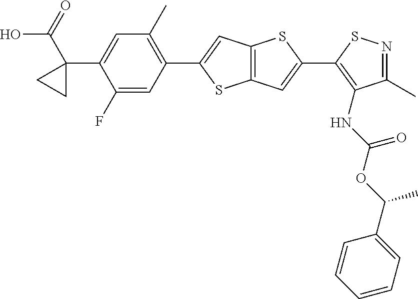 [SMILES: Cc1cc(C2(C(=O)O)CC2)c(F)cc1-c1cc2sc(-c3snc(C)c3NC(=O)O[C@H](C)c3ccccc3)cc2s1] |
| IT344 | 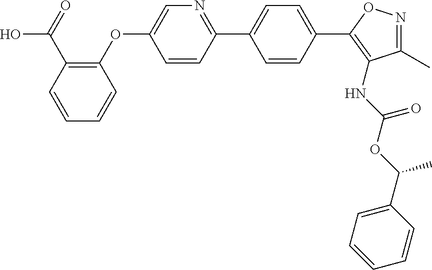 [SMILES: Cc1noc(-c2ccc(-c3ccc(Oc4ccccc4C(=O)O)cn3)cc2)c1NC(=O)O[C@H](C)c1ccccc1] |
| IT345 | 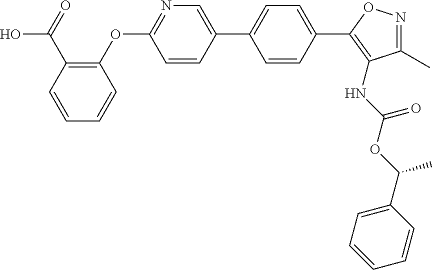 [SMILES: Cc1noc(-c2ccc(-c3ccc(Oc4ccccc4C(=O)O)nc3)cc2)c1NC(=O)O[C@H](C)c1ccccc1] |
| IT346 | 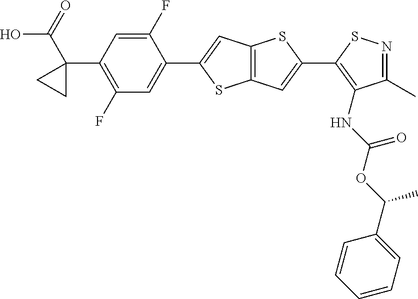 [SMILES: Cc1nsc(-c2cc3sc(-c4cc(F)c(C5(C(=O)O)CC5)cc4F)cc3s2)c1NC(=O)O[C@H](C)c1ccccc1] |
| IT347 | 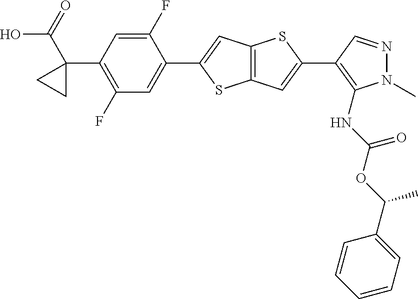 [SMILES: C[C@@H](OC(=O)Nc1c(-c2cc3sc(-c4cc(F)c(C5(C(=O)O)CC5)cc4F)cc3s2)cnn1C)c1ccccc1] |
| IT348 | 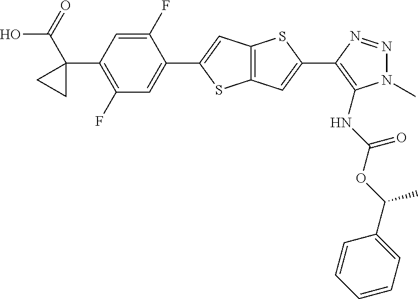 [SMILES: C[C@@H](OC(=O)Nc1c(-c2cc3sc(-c4cc(F)c(C5(C(=O)O)CC5)cc4F)cc3s2)nnn1C)c1ccccc1] |
| IT349 | 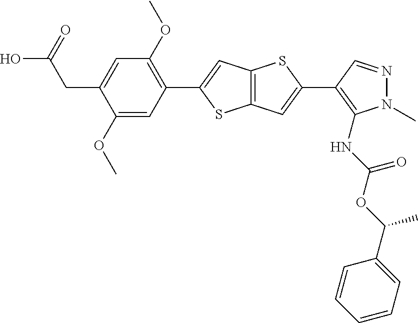 [SMILES: COc1cc(-c2cc3sc(-c4cnn(C)c4NC(=O)O[C@H](C)c4ccccc4)cc3s2)c(OC)cc1CC(=O)O] |
| IT350 | 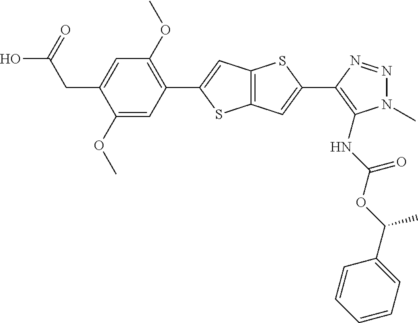 [SMILES: COc1cc(-c2cc3sc(-c4nnn(C)c4NC(=O)O[C@H](C)c4ccccc4)cc3s2)c(OC)cc1CC(=O)O] |
| IT351 | 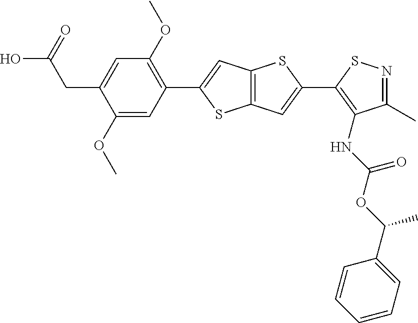 [SMILES: COc1cc(-c2cc3sc(-c4snc(C)c4NC(=O)O[C@H](C)c4ccccc4)cc3s2)c(OC)cc1CC(=O)O] |
| IT352 | 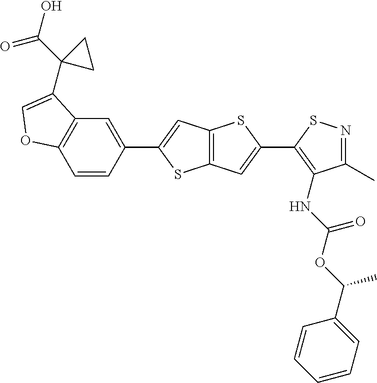 [SMILES: Cc1nsc(-c2cc3sc(-c4ccc5occ(C6(C(=O)O)CC6)c5c4)cc3s2)c1NC(=O)O[C@H](C)c1ccccc1] |
| IT353 | 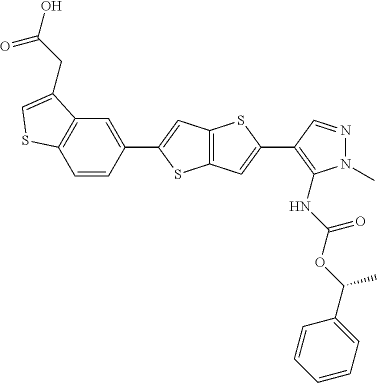 [SMILES: C[C@@H](OC(=O)Nc1c(-c2cc3sc(-c4ccc5scc(CC(=O)O)c5c4)cc3s2)cnn1C)c1ccccc1] |
| IT354 | 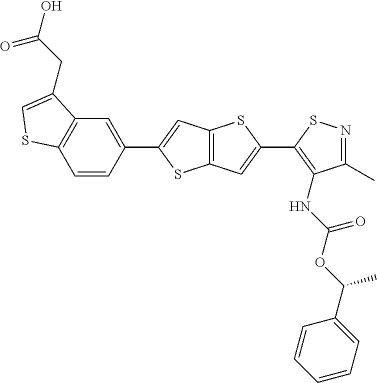 [SMILES: Cc1nsc(-c2cc3sc(-c4ccc5scc(CC(=O)O)c5c4)cc3s2)c1NC(=O)O[C@H](C)c1ccccc1] |
| IT355 | 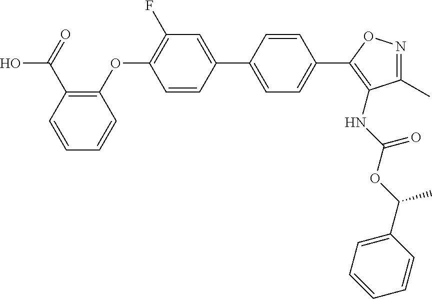 [SMILES: Cc1noc(-c2ccc(-c3ccc(Oc4ccccc4C(=O)O)c(F)c3)cc2)c1NC(=O)O[C@H](C)c1ccccc1] |
| IT356 | 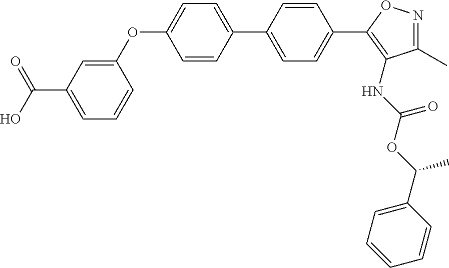 [SMILES: Cc1noc(-c2ccc(-c3ccc(Oc4cccc(C(=O)O)c4)cc3)cc2)c1NC(=O)O[C@H](C)c1ccccc1] |
| IT357 | 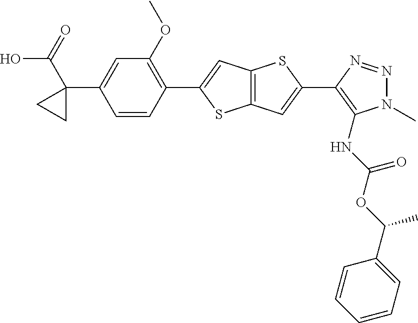 [SMILES: COc1cc(C2(C(=O)O)CC2)ccc1-c1cc2sc(-c3nnn(C)c3NC(=O)O[C@H](C)c3ccccc3)cc2s1] |
| IT358 | 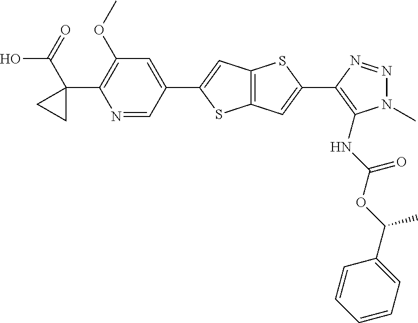 [SMILES: COc1cc(-c2cc3sc(-c4nnn(C)c4NC(=O)O[C@H](C)c4ccccc4)cc3s2)cnc1C1(C(=O)O)CC1] |
| IT359 | 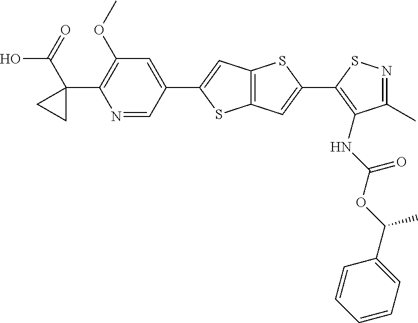 [SMILES: COc1cc(-c2cc3sc(-c4snc(C)c4NC(=O)O[C@H](C)c4ccccc4)cc3s2)cnc1C1(C(=O)O)CC1] |
| IT360 | 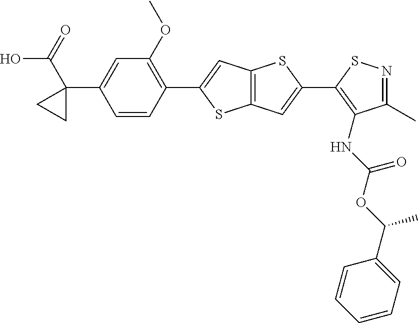 [SMILES: COc1cc(C2(C(=O)O)CC2)ccc1-c1cc2sc(-c3snc(C)c3NC(=O)O[C@H](C)c3ccccc3)cc2s1] |
| IT361 | 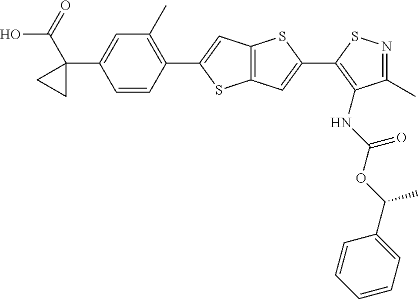 [SMILES: Cc1cc(C2(C(=O)O)CC2)ccc1-c1cc2sc(-c3snc(C)c3NC(=O)O[C@H](C)c3ccccc3)cc2s1] |
| IT362 | 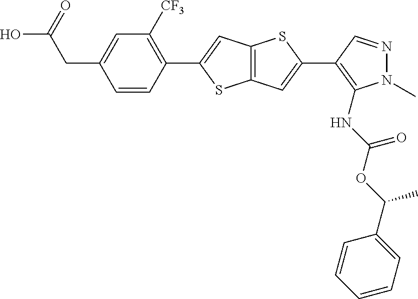 [SMILES: C[C@@H](OC(=O)Nc1c(-c2cc3sc(-c4ccc(CC(=O)O)cc4C(F)(F)F)cc3s2)cnn1C)c1ccccc1] |
| IT363 | 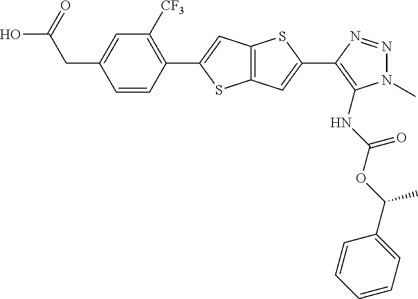 [SMILES: C[C@@H](OC(=O)Nc1c(-c2cc3sc(-c4ccc(CC(=O)O)cc4C(F)(F)F)cc3s2)nnn1C)c1ccccc1] |
| IT364 | 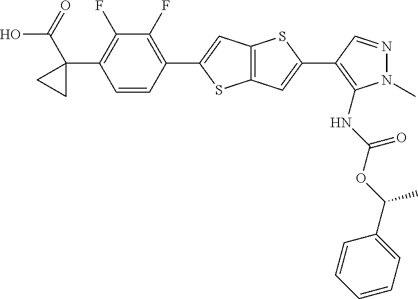 [SMILES: C[C@@H](OC(=O)Nc1c(-c2cc3sc(-c4ccc(C5(C(=O)O)CC5)c(F)c4F)cc3s2)cnn1C)c1ccccc1] |
| IT365 | 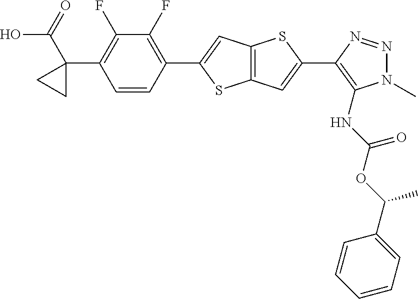 [SMILES: C[C@@H](OC(=O)Nc1c(-c2cc3sc(-c4ccc(C5(C(=O)O)CC5)c(F)c4F)cc3s2)nnn1C)c1ccccc1] |
| IT366 | 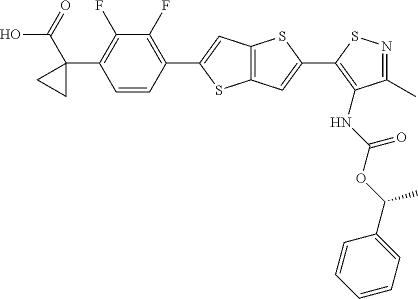 [SMILES: Cc1nsc(-c2cc3sc(-c4ccc(C5(C(=O)O)CC5)c(F)c4F)cc3s2)c1NC(=O)O[C@H](C)c1ccccc1] |
| IT367 | 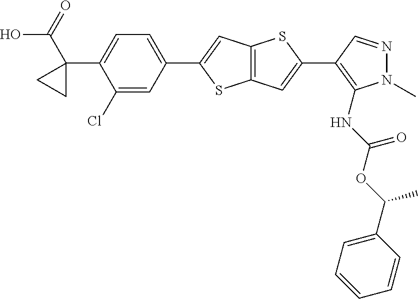 [SMILES: C[C@@H](OC(=O)Nc1c(-c2cc3sc(-c4ccc(C5(C(=O)O)CC5)c(Cl)c4)cc3s2)cnn1C)c1ccccc1] |
| IT368 | 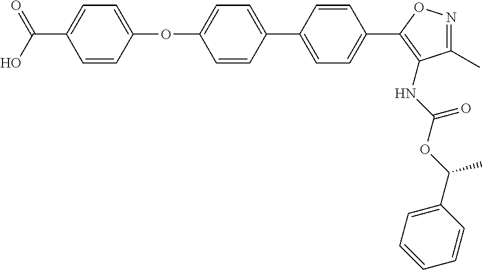 [SMILES: Cc1noc(-c2ccc(-c3ccc(Oc4ccc(C(=O)O)cc4)cc3)cc2)c1NC(=O)O[C@H](C)c1ccccc1] |
| IT369 | 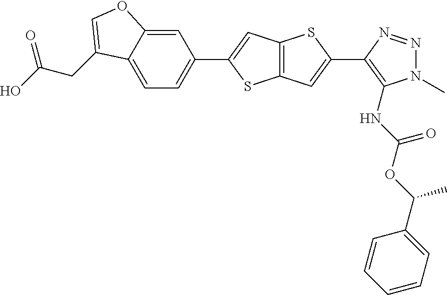 [SMILES: C[C@@H](OC(=O)Nc1c(-c2cc3sc(-c4ccc5c(CC(=O)O)coc5c4)cc3s2)nnn1C)c1ccccc1] |
| IT370 | 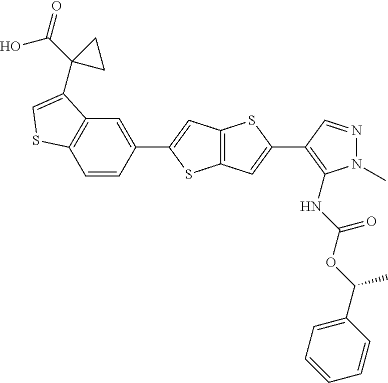 [SMILES: C[C@@H](OC(=O)Nc1c(-c2cc3sc(-c4ccc5scc(C6(C(=O)O)CC6)c5c4)cc3s2)cnn1C)c1ccccc1] |
| IT371 | 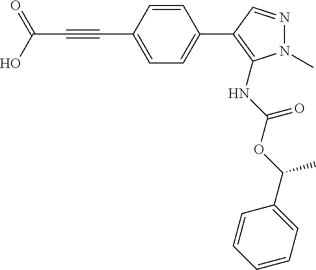 [SMILES: C[C@@H](OC(=O)Nc1c(-c2ccc(C#CC(=O)O)cc2)cnn1C)c1ccccc1] |
| IT372 | 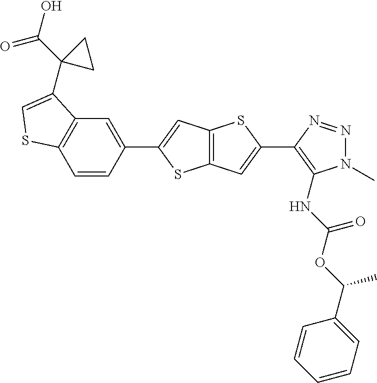 [SMILES: C[C@@H](OC(=O)Nc1c(-c2cc3sc(-c4ccc5scc(C6(C(=O)O)CC6)c5c4)cc3s2)nnn1C)c1ccccc1] |
| IT373 | 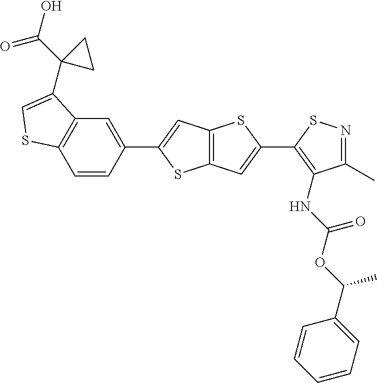 [SMILES: Cc1nsc(-c2cc3sc(-c4ccc5scc(C6(C(=O)O)CC6)c5c4)cc3s2)c1NC(=O)O[C@H](C)c1ccccc1] |
| IT374 | 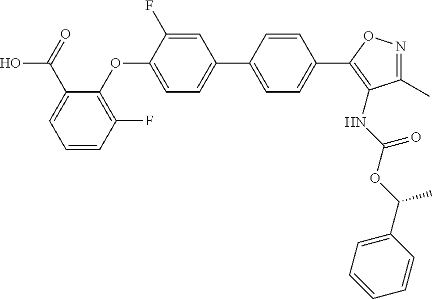 [SMILES: Cc1noc(-c2ccc(-c3ccc(Oc4c(F)cccc4C(=O)O)c(F)c3)cc2)c1NC(=O)O[C@H](C)c1ccccc1] |
| IT375 | 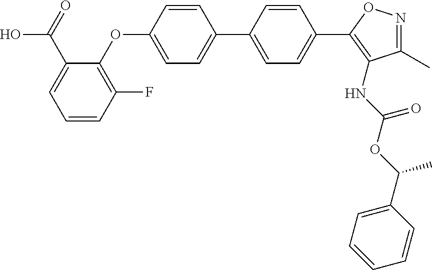 [SMILES: Cc1noc(-c2ccc(-c3ccc(Oc4c(F)cccc4C(=O)O)cc3)cc2)c1NC(=O)O[C@H](C)c1ccccc1] |
| IT376 | 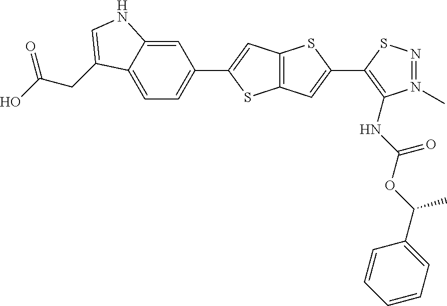 |
| IT377 | 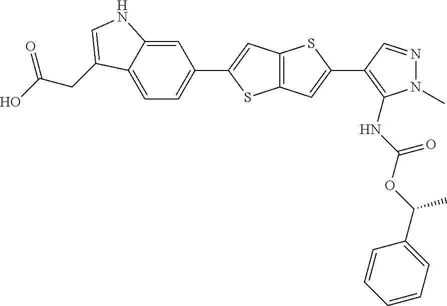 [SMILES: C[C@@H](OC(=O)Nc1c(-c2cc3sc(-c4ccc5c(CC(=O)O)c[nH]c5c4)cc3s2)cnn1C)c1ccccc1] |
| IT378 | 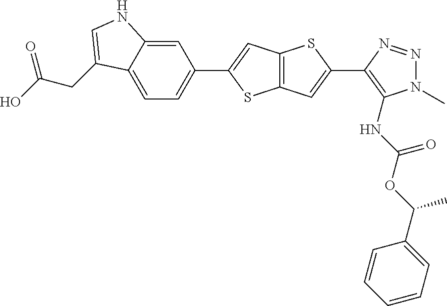 [SMILES: C[C@@H](OC(=O)Nc1c(-c2cc3sc(-c4ccc5c(CC(=O)O)c[nH]c5c4)cc3s2)nnn1C)c1ccccc1] |
| IT379 | 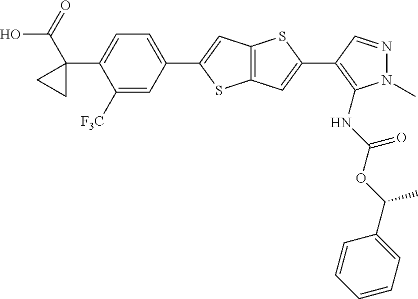 [SMILES: C[C@@H](OC(=O)Nc1c(-c2cc3sc(-c4ccc(C5(C(=O)O)CC5)c(C(F)(F)F)c4)cc3s2)cnn1C)c1ccccc1] |
| IT380 | 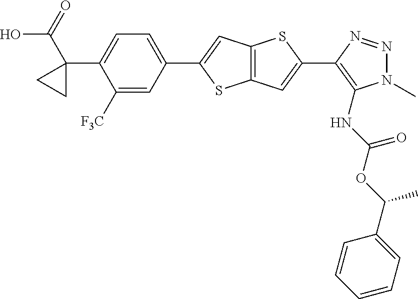 [SMILES: C[C@@H](OC(=O)Nc1c(-c2cc3sc(-c4ccc(C5(C(=O)O)CC5)c(C(F)(F)F)c4)cc3s2)nnn1C)c1ccccc1] |
| IT381 | 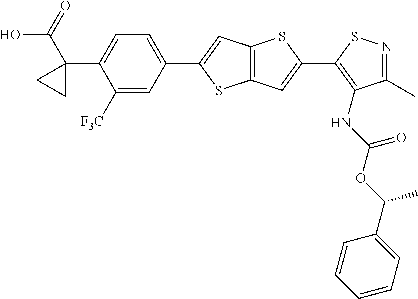 [SMILES: Cc1nsc(-c2cc3sc(-c4ccc(C5(C(=O)O)CC5)c(C(F)(F)F)c4)cc3s2)c1NC(=O)O[C@H](C)c1ccccc1] |
| IT382 | 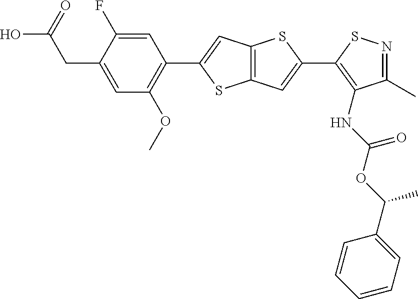 [SMILES: COc1cc(CC(=O)O)c(F)cc1-c1cc2sc(-c3snc(C)c3NC(=O)O[C@H](C)c3ccccc3)cc2s1] |
| IT383 | 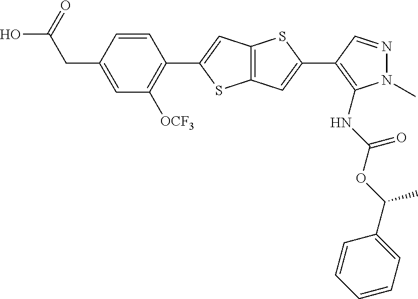 [SMILES: C[C@@H](OC(=O)Nc1c(-c2cc3sc(-c4ccc(CC(=O)O)cc4OC(F)(F)F)cc3s2)cnn1C)c1ccccc1] |
| IT384 | 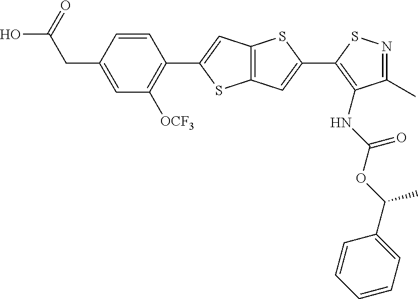 [SMILES: Cc1nsc(-c2cc3sc(-c4ccc(CC(=O)O)cc4OC(F)(F)F)cc3s2)c1NC(=O)O[C@H](C)c1ccccc1] |
| IT385 | 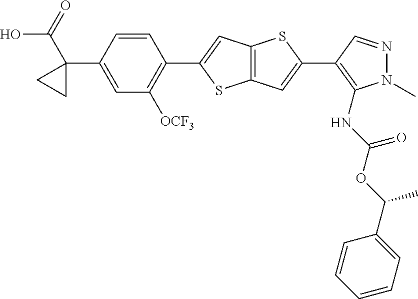 [SMILES: C[C@@H](OC(=O)Nc1c(-c2cc3sc(-c4ccc(C5(C(=O)O)CC5)cc4OC(F)(F)F)cc3s2)cnn1C)c1ccccc1] |
| IT386 | 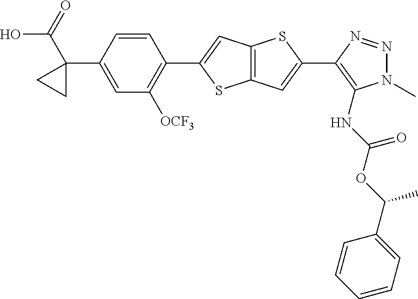 [SMILES: C[C@@H](OC(=O)Nc1c(-c2cc3sc(-c4ccc(C5(C(=O)O)CC5)cc4OC(F)(F)F)cc3s2)nnn1C)c1ccccc1] |
| IT387 | 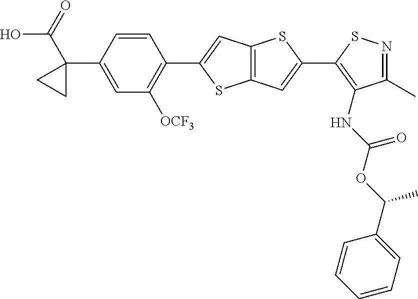 [SMILES: Cc1nsc(-c2cc3sc(-c4ccc(C5(C(=O)O)CC5)cc4OC(F)(F)F)cc3s2)c1NC(=O)O[C@H](C)c1ccccc1] |
| IT388 | 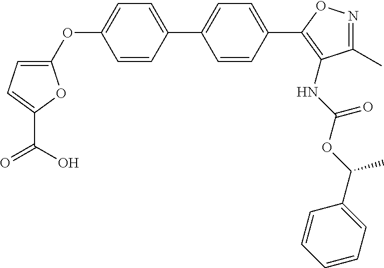 [SMILES: Cc1noc(-c2ccc(-c3ccc(Oc4ccc(C(=O)O)o4)cc3)cc2)c1NC(=O)O[C@H](C)c1ccccc1] |
| IT389 | 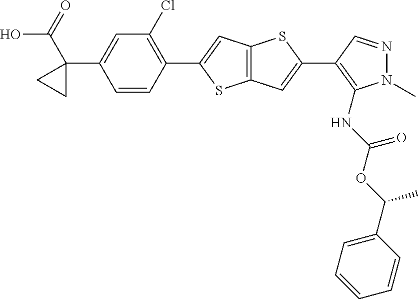 [SMILES: C[C@@H](OC(=O)Nc1c(-c2cc3sc(-c4ccc(C5(C(=O)O)CC5)cc4Cl)cc3s2)cnn1C)c1ccccc1] |
| IT390 | 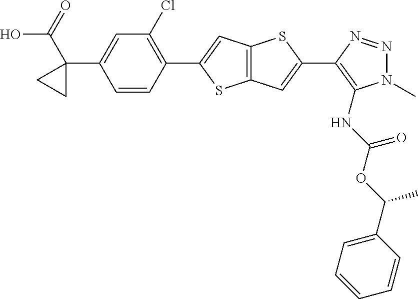 [SMILES: C[C@@H](OC(=O)Nc1c(-c2cc3sc(-c4ccc(C5(C(=O)O)CC5)cc4Cl)cc3s2)nnn1C)c1ccccc1] |
| IT391 | 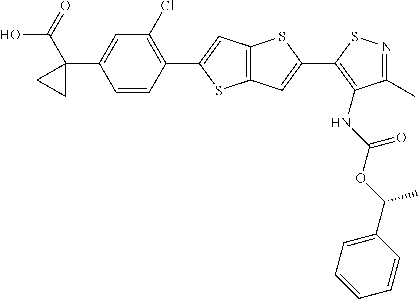 [SMILES: Cc1nsc(-c2cc3sc(-c4ccc(C5(C(=O)O)CC5)cc4Cl)cc3s2)c1NC(=O)O[C@H](C)c1ccccc1] |
| IT392 | 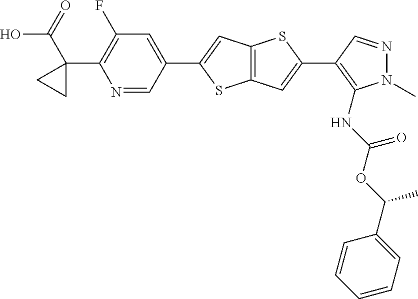 [SMILES: C[C@@H](OC(=O)Nc1c(-c2cc3sc(-c4cnc(C5(C(=O)O)CC5)c(F)c4)cc3s2)cnn1C)c1ccccc1] |
| IT393 | 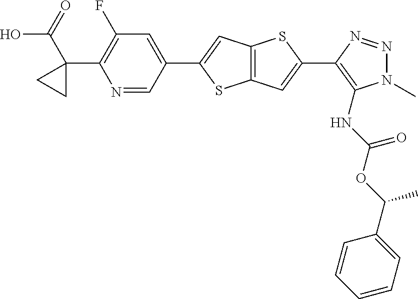 [SMILES: C[C@@H](OC(=O)Nc1c(-c2cc3sc(-c4cnc(C5(C(=O)O)CC5)c(F)c4)cc3s2)nnn1C)c1ccccc1] |
| IT394 | 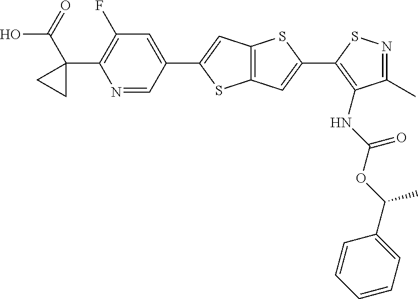 [SMILES: Cc1nsc(-c2cc3sc(-c4cnc(C5(C(=O)O)CC5)c(F)c4)cc3s2)c1NC(=O)O[C@H](C)c1ccccc1] |
| IT395 | 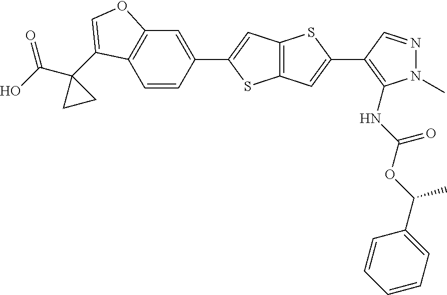 [SMILES: C[C@@H](OC(=O)Nc1c(-c2cc3sc(-c4ccc5c(C6(C(=O)O)CC6)coc5c4)cc3s2)cnn1C)c1ccccc1] |
| IT396 | 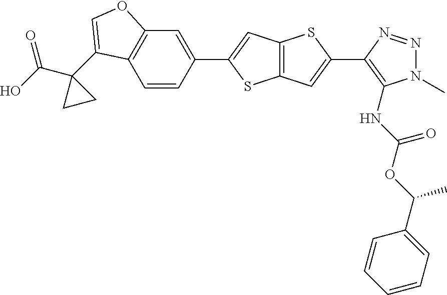 [SMILES: C[C@@H](OC(=O)Nc1c(-c2cc3sc(-c4ccc5c(C6(C(=O)O)CC6)coc5c4)cc3s2)nnn1C)c1ccccc1] |
| IT397 | 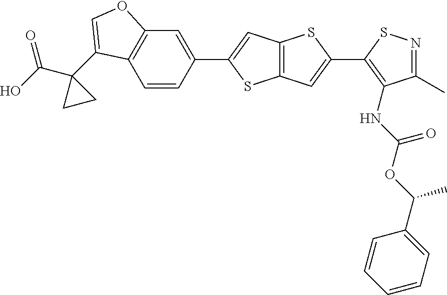 [SMILES: Cc1nsc(-c2cc3sc(-c4ccc5c(C6(C(=O)O)CC6)coc5c4)cc3s2)c1NC(=O)O[C@H](C)c1ccccc1] |
| IT398 | 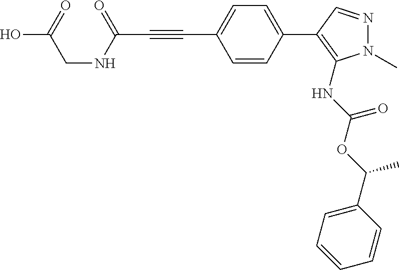 [SMILES: C[C@@H](OC(=O)Nc1c(-c2ccc(C#CC(=O)NCC(=O)O)cc2)cnn1C)c1ccccc1] |
| IT399 | 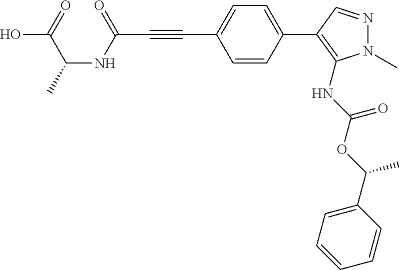 [SMILES: C[C@@H](NC(=O)C#Cc1ccc(-c2cnn(C)c2NC(=O)O[C@H](C)c2ccccc2)cc1)C(=O)O] |
| IT400 | 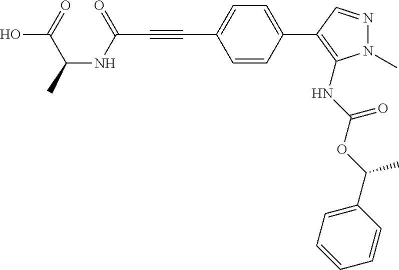 [SMILES: C[C@H](NC(=O)C#Cc1ccc(-c2cnn(C)c2NC(=O)O[C@H](C)c2ccccc2)cc1)C(=O)O] |
| IT401 | 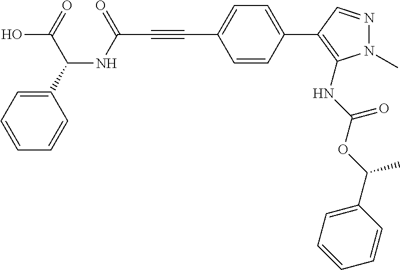 [SMILES: C[C@@H](OC(=O)Nc1c(-c2ccc(C#CC(=O)N[C@@H](C(=O)O)c3ccccc3)cc2)cnn1C)c1ccccc1] |
| IT402 | 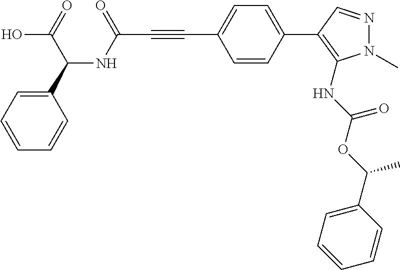 [SMILES: C[C@@H](OC(=O)Nc1c(-c2ccc(C#CC(=O)N[C@H](C(=O)O)c3ccccc3)cc2)cnn1C)c1ccccc1] |
| IT403 | 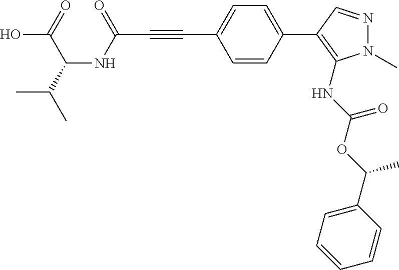 [SMILES: CC(C)[C@@H](NC(=O)C#Cc1ccc(-c2cnn(C)c2NC(=O)O[C@H](C)c2ccccc2)cc1)C(=O)O] |
| IT404 | 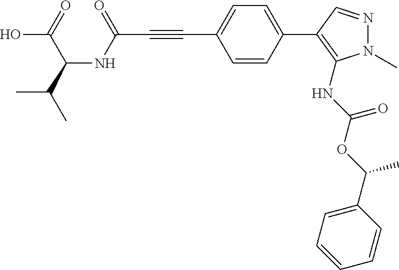 [SMILES: CC(C)[C@H](NC(=O)C#Cc1ccc(-c2cnn(C)c2NC(=O)O[C@H](C)c2ccccc2)cc1)C(=O)O] |
| IT405 | 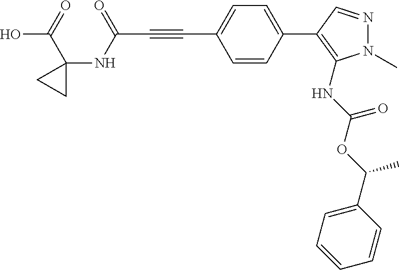 [SMILES: C[C@@H](OC(=O)Nc1c(-c2ccc(C#CC(=O)NC3(C(=O)O)CC3)cc2)cnn1C)c1ccccc1] |
| IT406 | 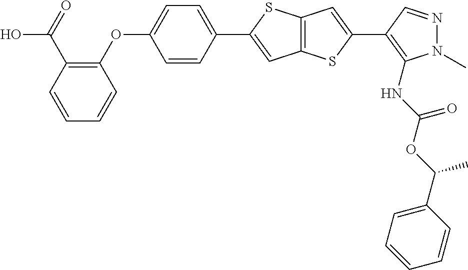 [SMILES: C[C@@H](OC(=O)Nc1c(-c2cc3sc(-c4ccc(Oc5ccccc5C(=O)O)cc4)cc3s2)cnn1C)c1ccccc1] |
| IT407 | 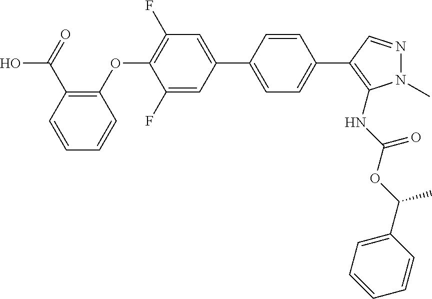 [SMILES: C[C@@H](OC(=O)Nc1c(-c2ccc(-c3cc(F)c(Oc4ccccc4C(=O)O)c(F)c3)cc2)cnn1C)c1ccccc1] |
| IT408 | 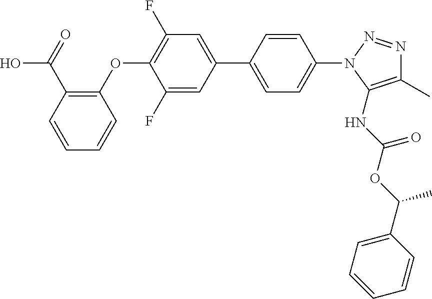 [SMILES: Cc1nnn(-c2ccc(-c3cc(F)c(Oc4ccccc4C(=O)O)c(F)c3)cc2)c1NC(=O)O[C@H](C)c1ccccc1] |
| IT409 | 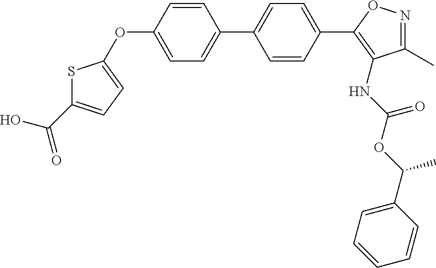 [SMILES: Cc1noc(-c2ccc(-c3ccc(Oc4ccc(C(=O)O)s4)cc3)cc2)c1NC(=O)O[C@H](C)c1ccccc1] |
| IT410 | 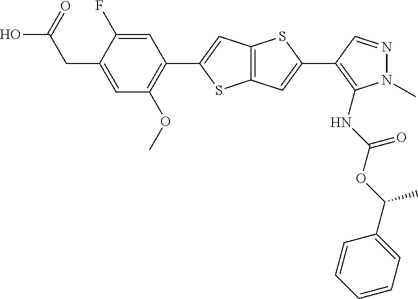 [SMILES: COc1cc(CC(=O)O)c(F)cc1-c1cc2sc(-c3cnn(C)c3NC(=O)O[C@H](C)c3ccccc3)cc2s1] |
| IT411 | 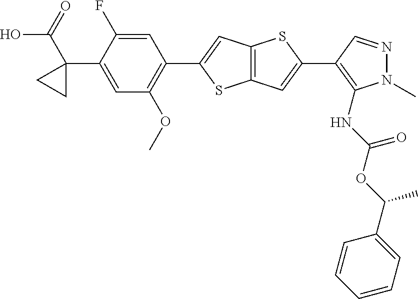 [SMILES: COc1cc(C2(C(=O)O)CC2)c(F)cc1-c1cc2sc(-c3cnn(C)c3NC(=O)O[C@H](C)c3ccccc3)cc2s1] |
| IT412 | 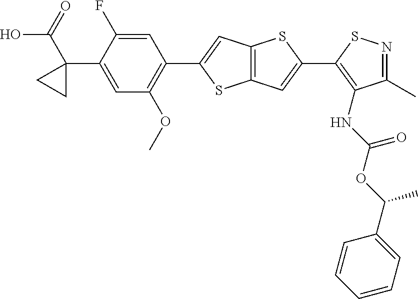 [SMILES: COc1cc(C2(C(=O)O)CC2)c(F)cc1-c1cc2sc(-c3snc(C)c3NC(=O)O[C@H](C)c3ccccc3)cc2s1] |
| IT413 | 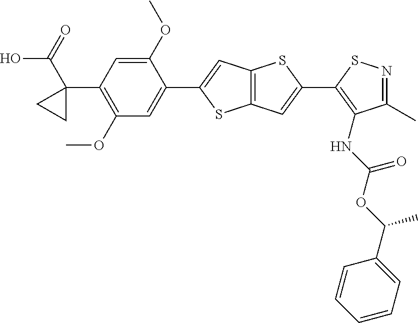 [SMILES: COc1cc(C2(C(=O)O)CC2)c(OC)cc1-c1cc2sc(-c3snc(C)c3NC(=O)O[C@H](C)c3ccccc3)cc2s1] |
| IT414 | 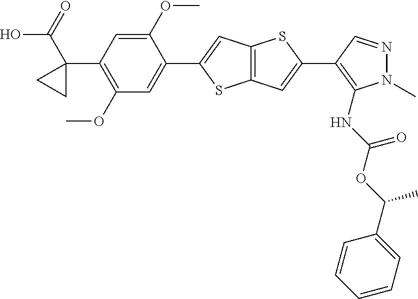 [SMILES: COc1cc(C2(C(=O)O)CC2)c(OC)cc1-c1cc2sc(-c3cnn(C)c3NC(=O)O[C@H](C)c3ccccc3)cc2s1] |
| IT415 | 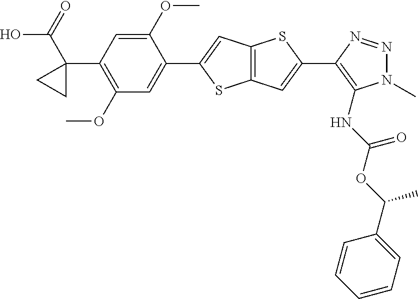 [SMILES: COc1cc(C2(C(=O)O)CC2)c(OC)cc1-c1cc2sc(-c3nnn(C)c3NC(=O)O[C@H](C)c3ccccc3)cc2s1] |
| IT416 | 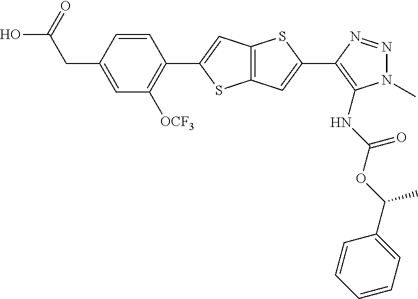 [SMILES: C[C@@H](OC(=O)Nc1c(-c2cc3sc(-c4ccc(CC(=O)O)cc4OC(F)(F)F)cc3s2)nnn1C)c1ccccc1] |
| IT417 | 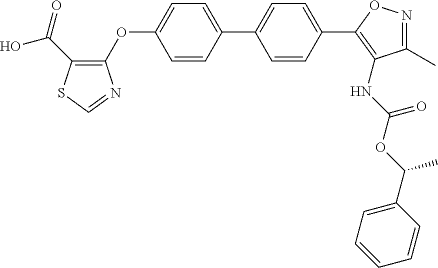 [SMILES: Cc1noc(-c2ccc(-c3ccc(Oc4ncsc4C(=O)O)cc3)cc2)c1NC(=O)O[C@H](C)c1ccccc1] |
| IT418 | 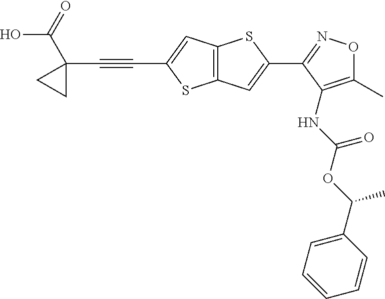 [SMILES: Cc1onc(-c2cc3sc(C#CC4(C(=O)O)CC4)cc3s2)c1NC(=O)O[C@H](C)c1ccccc1] |
| IT419 | 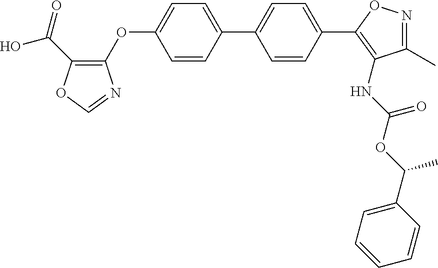 [SMILES: Cc1noc(-c2ccc(-c3ccc(Oc4ncoc4C(=O)O)cc3)cc2)c1NC(=O)O[C@H](C)c1ccccc1] |
| IT420 | 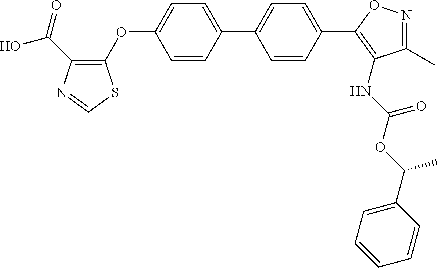 [SMILES: Cc1noc(-c2ccc(-c3ccc(Oc4scnc4C(=O)O)cc3)cc2)c1NC(=O)O[C@H](C)c1ccccc1] |
| IT421 | 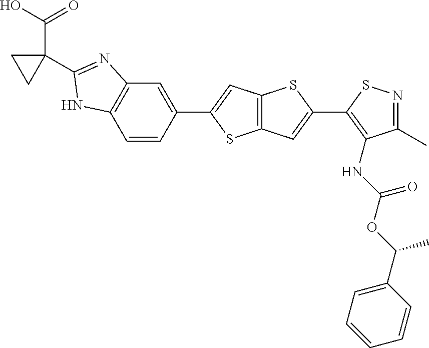 [SMILES: Cc1nsc(-c2cc3sc(-c4ccc5[nH]c(C6(C(=O)O)CC6)nc5c4)cc3s2)c1NC(=O)O[C@H](C)c1ccccc1] |
| IT422 | 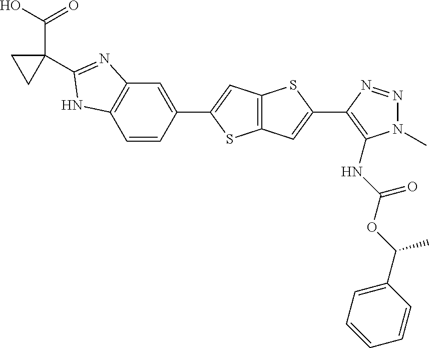 [SMILES: C[C@@H](OC(=O)Nc1c(-c2cc3sc(-c4ccc5[nH]c(C6(C(=O)O)CC6)nc5c4)cc3s2)nnn1C)c1ccccc1] |
| IT423 | 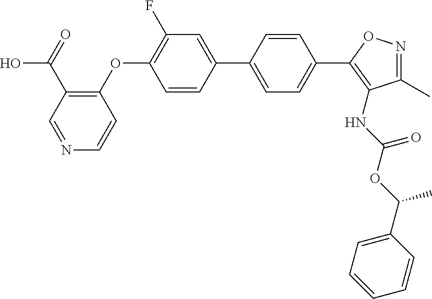 [SMILES: Cc1noc(-c2ccc(-c3ccc(Oc4ccncc4C(=O)O)c(F)c3)cc2)c1NC(=O)O[C@H](C)c1ccccc1] |
| IT424 | 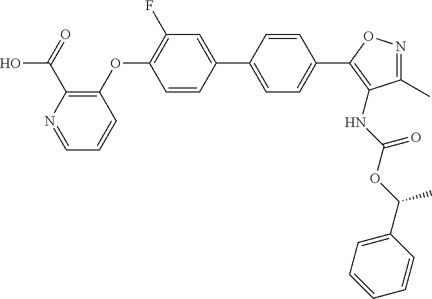 [SMILES: Cc1noc(-c2ccc(-c3ccc(Oc4cccnc4C(=O)O)c(F)c3)cc2)c1NC(=O)O[C@H](C)c1ccccc1] |
| IT425 | 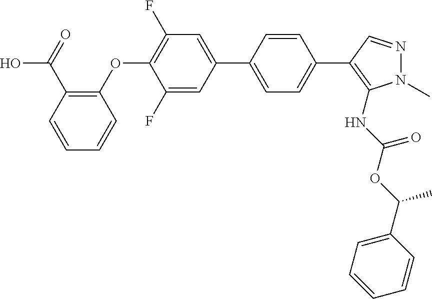 [SMILES: C[C@@H](OC(=O)Nc1c(-c2ccc(-c3cc(F)c(Oc4ccccc4C(=O)O)c(F)c3)cc2)cnn1C)c1ccccc1] |
| IT426 | 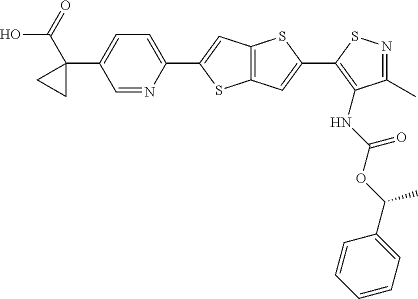 [SMILES: Cc1nsc(-c2cc3sc(-c4ccc(C5(C(=O)O)CC5)cn4)cc3s2)c1NC(=O)O[C@H](C)c1ccccc1] |
| IT427 | 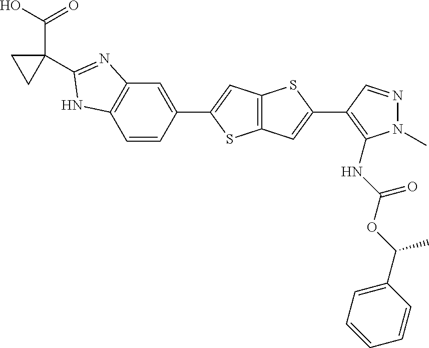 [SMILES: C[C@@H](OC(=O)Nc1c(-c2cc3sc(-c4ccc5[nH]c(C6(C(=O)O)CC6)nc5c4)cc3s2)cnn1C)c1ccccc1] |
| IT428 | 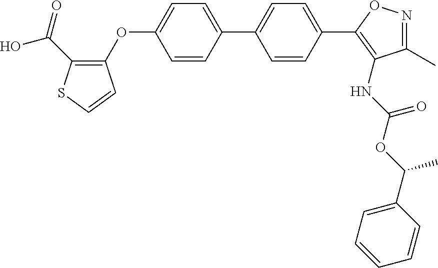 [SMILES: Cc1noc(-c2ccc(-c3ccc(Oc4ccsc4C(=O)O)cc3)cc2)c1NC(=O)O[C@H](C)c1ccccc1] |
| IT429 | 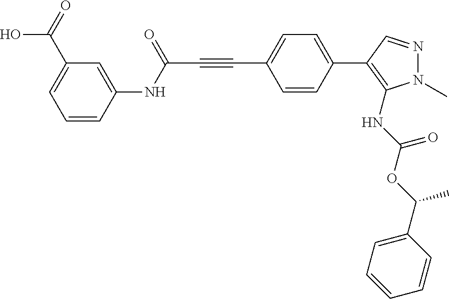 [SMILES: C[C@@H](OC(=O)Nc1c(-c2ccc(C#CC(=O)Nc3cccc(C(=O)O)c3)cc2)cnn1C)c1ccccc1] |
| IT430 | 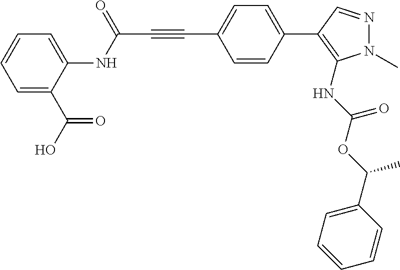 [SMILES: C[C@@H](OC(=O)Nc1c(-c2ccc(C#CC(=O)Nc3ccccc3C(=O)O)cc2)cnn1C)c1ccccc1] |
| IT431 | 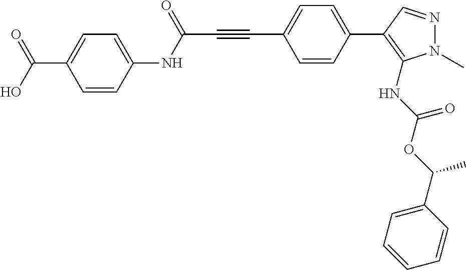 [SMILES: C[C@@H](OC(=O)Nc1c(-c2ccc(C#CC(=O)Nc3ccc(C(=O)O)cc3)cc2)cnn1C)c1ccccc1] |
| IT432 | 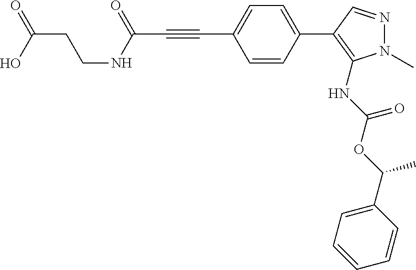 [SMILES: C[C@@H](OC(=O)Nc1c(-c2ccc(C#CC(=O)NCCC(=O)O)cc2)cnn1C)c1ccccc1] |
| IT433 | 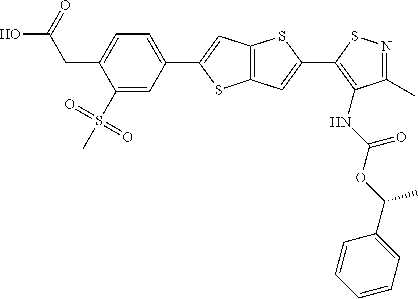 [SMILES: Cc1nsc(-c2cc3sc(-c4ccc(CC(=O)O)c(S(C)(=O)=O)c4)cc3s2)c1NC(=O)O[C@H](C)c1ccccc1] |
| IT434 | 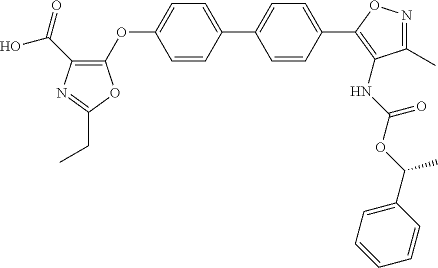 [SMILES: CCc1nc(C(=O)O)c(Oc2ccc(-c3ccc(-c4onc(C)c4NC(=O)O[C@H](C)c4ccccc4)cc3)cc2)o1] |
| IT435 | 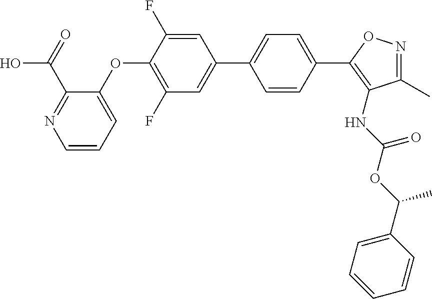 [SMILES: Cc1noc(-c2ccc(-c3cc(F)c(Oc4cccnc4C(=O)O)c(F)c3)cc2)c1NC(=O)O[C@H](C)c1ccccc1] |
| IT436 | 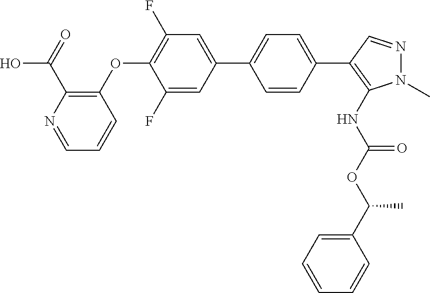 [SMILES: C[C@@H](OC(=O)Nc1c(-c2ccc(-c3cc(F)c(Oc4cccnc4C(=O)O)c(F)c3)cc2)cnn1C)c1ccccc1] |
| IT437 | 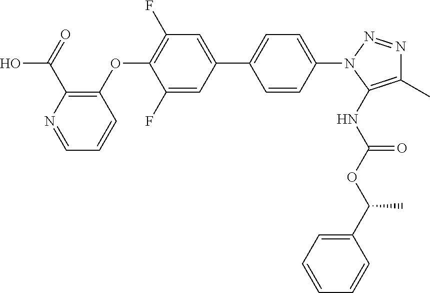 [SMILES: Cc1nnn(-c2ccc(-c3cc(F)c(Oc4cccnc4C(=O)O)c(F)c3)cc2)c1NC(=O)O[C@H](C)c1ccccc1] |
| IT438 | 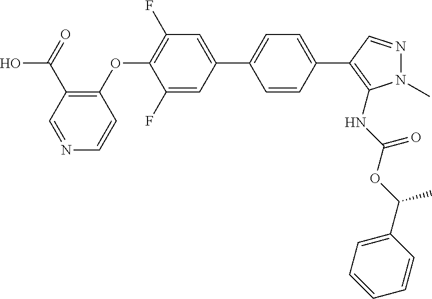 [SMILES: C[C@@H](OC(=O)Nc1c(-c2ccc(-c3cc(F)c(Oc4ccncc4C(=O)O)c(F)c3)cc2)cnn1C)c1ccccc1] |
| IT439 | 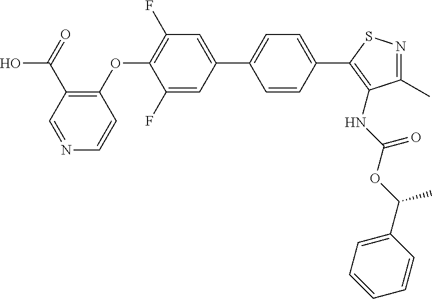 [SMILES: Cc1nsc(-c2ccc(-c3cc(F)c(Oc4ccncc4C(=O)O)c(F)c3)cc2)c1NC(=O)O[C@H](C)c1ccccc1] |
| IT440 | 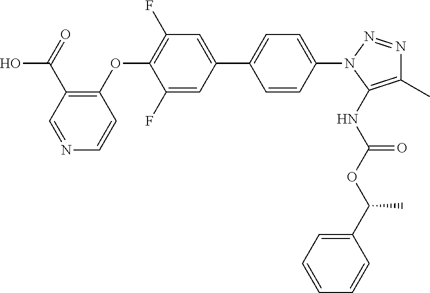 [SMILES: Cc1nnn(-c2ccc(-c3cc(F)c(Oc4ccncc4C(=O)O)c(F)c3)cc2)c1NC(=O)O[C@H](C)c1ccccc1] |
| IT441 | 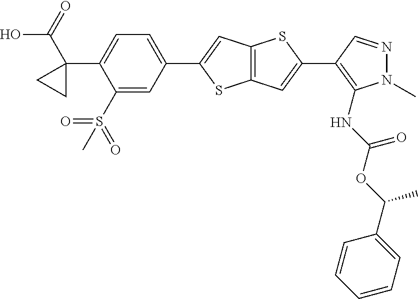 [SMILES: C[C@@H](OC(=O)Nc1c(-c2cc3sc(-c4ccc(C5(C(=O)O)CC5)c(S(C)(=O)=O)c4)cc3s2)cnn1C)c1ccccc1] |
| IT442 | 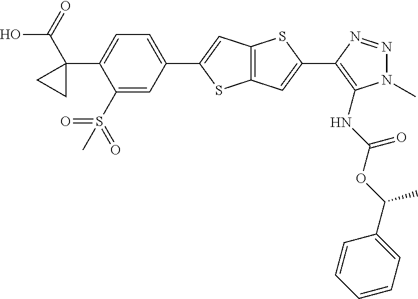 [SMILES: C[C@@H](OC(=O)Nc1c(-c2cc3sc(-c4ccc(C5(C(=O)O)CC5)c(S(C)(=O)=O)c4)cc3s2)nnn1C)c1ccccc1] |
| IT443 | 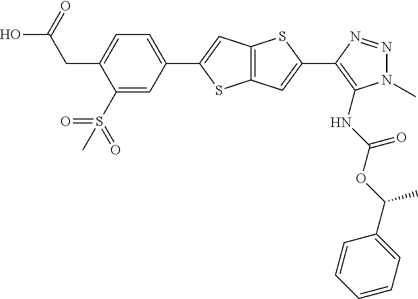 [SMILES: C[C@@H](OC(=O)Nc1c(-c2cc3sc(-c4ccc(CC(=O)O)c(S(C)(=O)=O)c4)cc3s2)nnn1C)c1ccccc1] |
| IT444 | 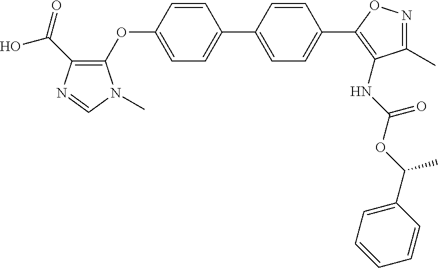 [SMILES: Cc1noc(-c2ccc(-c3ccc(Oc4c(C(=O)O)ncn4C)cc3)cc2)c1NC(=O)O[C@H](C)c1ccccc1] |
| IT445 | 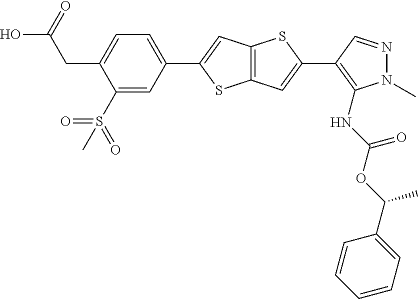 [SMILES: C[C@@H](OC(=O)Nc1c(-c2cc3sc(-c4ccc(CC(=O)O)c(S(C)(=O)=O)c4)cc3s2)cnn1C)c1ccccc1] |
| IT446 | 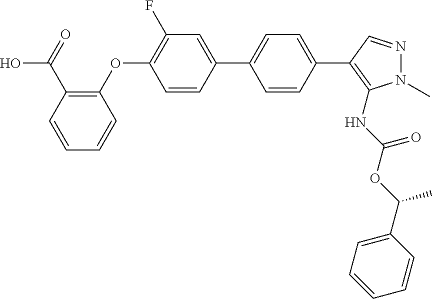 [SMILES: C[C@@H](OC(=O)Nc1c(-c2ccc(-c3ccc(Oc4ccccc4C(=O)O)c(F)c3)cc2)cnn1C)c1ccccc1] |
| IT447 | 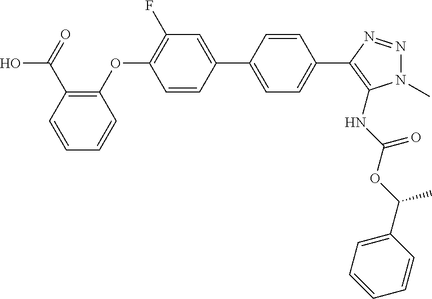 [SMILES: C[C@@H](OC(=O)Nc1c(-c2ccc(-c3ccc(Oc4ccccc4C(=O)O)c(F)c3)cc2)nnn1C)c1ccccc1] |
| IT448 | 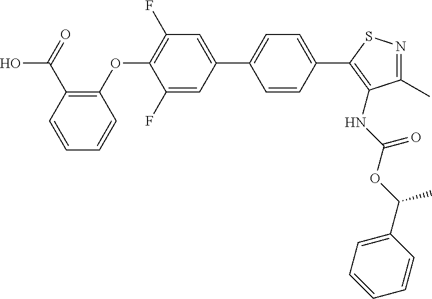 [SMILES: Cc1nsc(-c2ccc(-c3cc(F)c(Oc4ccccc4C(=O)O)c(F)c3)cc2)c1NC(=O)O[C@H](C)c1ccccc1] |
| IT449 | 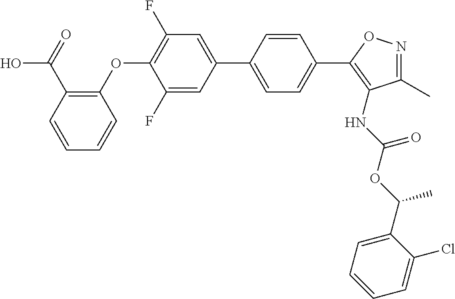 [SMILES: Cc1noc(-c2ccc(-c3cc(F)c(Oc4ccccc4C(=O)O)c(F)c3)cc2)c1NC(=O)O[C@H](C)c1ccccc1Cl] |
| IT450 | 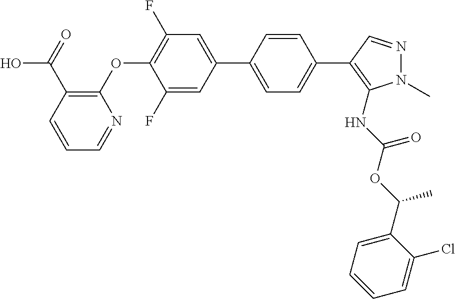 [SMILES: C[C@@H](OC(=O)Nc1c(-c2ccc(-c3cc(F)c(Oc4ncccc4C(=O)O)c(F)c3)cc2)cnn1C)c1ccccc1Cl] |
| IT451 | 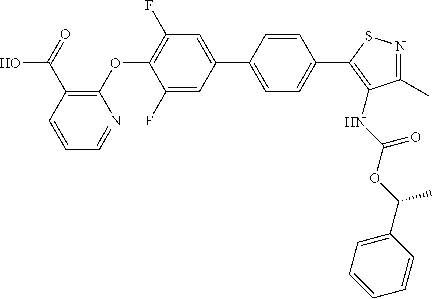 [SMILES: Cc1nsc(-c2ccc(-c3cc(F)c(Oc4ncccc4C(=O)O)c(F)c3)cc2)c1NC(=O)O[C@H](C)c1ccccc1] |
| IT452 | 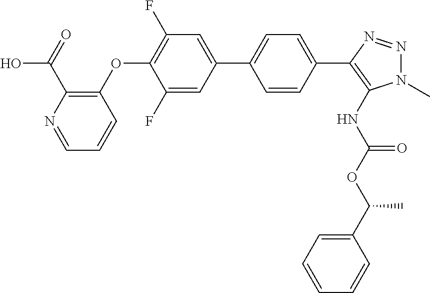 [SMILES: C[C@@H](OC(=O)Nc1c(-c2ccc(-c3cc(F)c(Oc4cccnc4C(=O)O)c(F)c3)cc2)nnn1C)c1ccccc1] |
| IT453 | 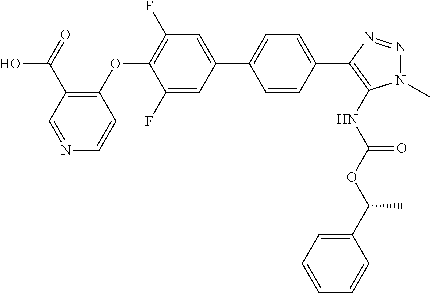 [SMILES: C[C@@H](OC(=O)Nc1c(-c2ccc(-c3cc(F)c(Oc4ccncc4C(=O)O)c(F)c3)cc2)nnn1C)c1ccccc1] |
| IT454 | 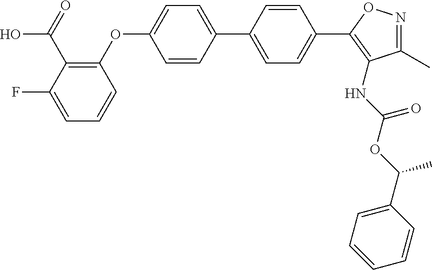 [SMILES: Cc1noc(-c2ccc(-c3ccc(Oc4cccc(F)c4C(=O)O)cc3)cc2)c1NC(=O)O[C@H](C)c1ccccc1] |
| IT455 | 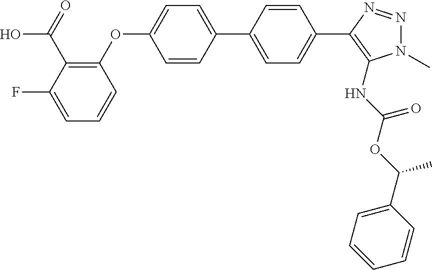 [SMILES: C[C@@H](OC(=O)Nc1c(-c2ccc(-c3ccc(Oc4cccc(F)c4C(=O)O)cc3)cc2)nnn1C)c1ccccc1] |
| IT456 | 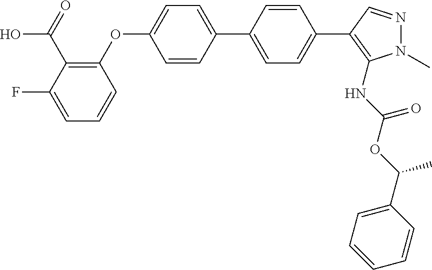 [SMILES: C[C@@H](OC(=O)Nc1c(-c2ccc(-c3ccc(Oc4cccc(F)c4C(=O)O)cc3)cc2)cnn1C)c1ccccc1] |
| IT457 | 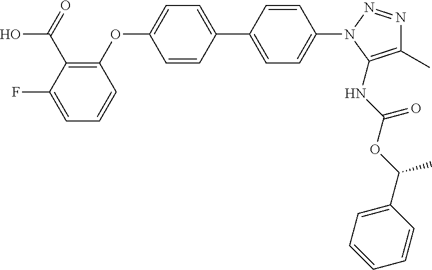 [SMILES: Cc1nnn(-c2ccc(-c3ccc(Oc4cccc(F)c4C(=O)O)cc3)cc2)c1NC(=O)O[C@H](C)c1ccccc1] |
| IT458 | 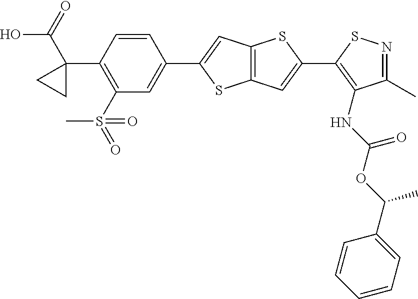 [SMILES: Cc1nsc(-c2cc3sc(-c4ccc(C5(C(=O)O)CC5)c(S(C)(=O)=O)c4)cc3s2)c1NC(=O)O[C@H](C)c1ccccc1] |
| IT459 | 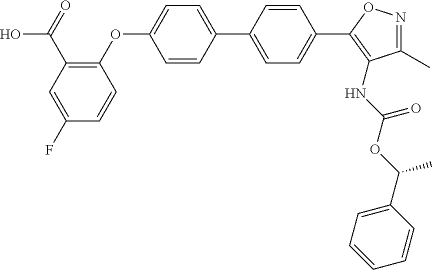 [SMILES: Cc1noc(-c2ccc(-c3ccc(Oc4ccc(F)cc4C(=O)O)cc3)cc2)c1NC(=O)O[C@H](C)c1ccccc1] |
| IT460 | 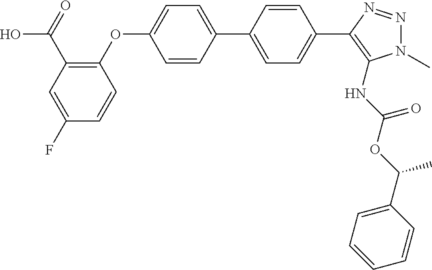 [SMILES: C[C@@H](OC(=O)Nc1c(-c2ccc(-c3ccc(Oc4ccc(F)cc4C(=O)O)cc3)cc2)nnn1C)c1ccccc1] |
| IT461 | 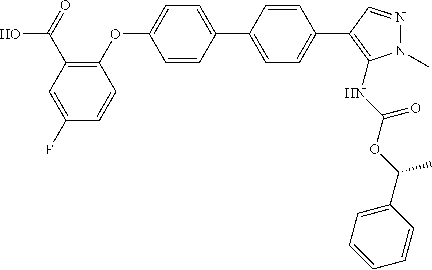 [SMILES: C[C@@H](OC(=O)Nc1c(-c2ccc(-c3ccc(Oc4ccc(F)cc4C(=O)O)cc3)cc2)cnn1C)c1ccccc1] |
| IT462 | 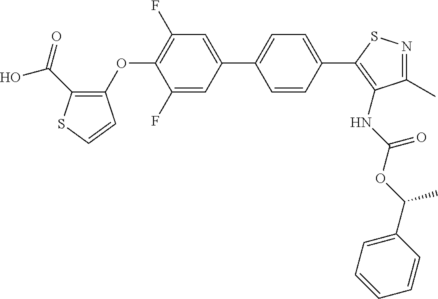 [SMILES: Cc1nsc(-c2ccc(-c3cc(F)c(Oc4ccsc4C(=O)O)c(F)c3)cc2)c1NC(=O)O[C@H](C)c1ccccc1] |
| IT463 | 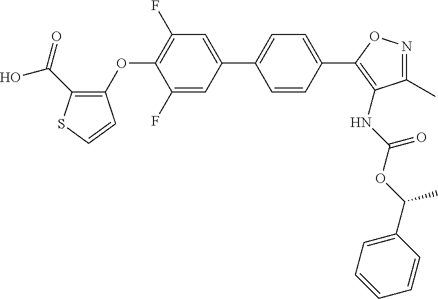 [SMILES: Cc1noc(-c2ccc(-c3cc(F)c(Oc4ccsc4C(=O)O)c(F)c3)cc2)c1NC(=O)O[C@H](C)c1ccccc1] |
| IT464 | 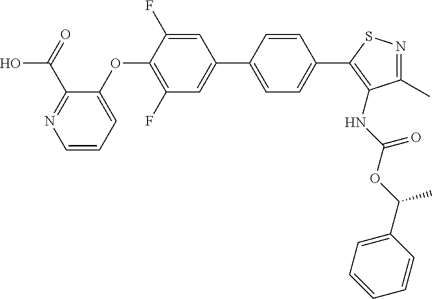 [SMILES: Cc1nsc(-c2ccc(-c3cc(F)c(Oc4cccnc4C(=O)O)c(F)c3)cc2)c1NC(=O)O[C@H](C)c1ccccc1] |
| IT465 | 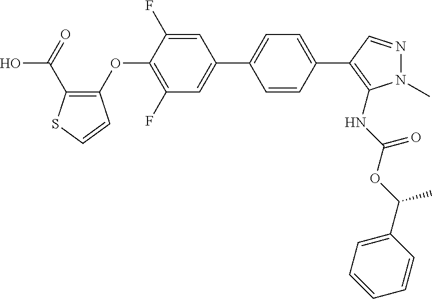 [SMILES: C[C@@H](OC(=O)Nc1c(-c2ccc(-c3cc(F)c(Oc4ccsc4C(=O)O)c(F)c3)cc2)cnn1C)c1ccccc1] |
| IT466 | 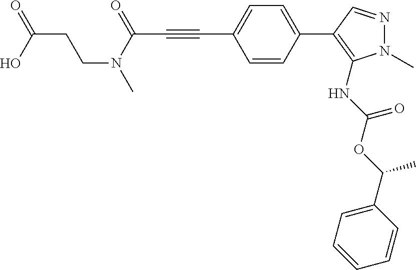 [SMILES: C[C@@H](OC(=O)Nc1c(-c2ccc(C#CC(=O)N(C)CCC(=O)O)cc2)cnn1C)c1ccccc1] |
| IT467 | 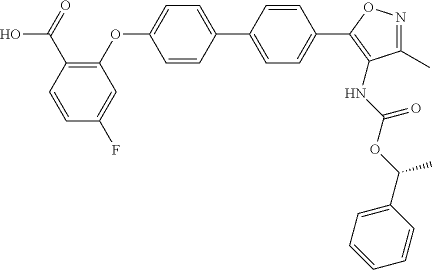 [SMILES: Cc1noc(-c2ccc(-c3ccc(Oc4cc(F)ccc4C(=O)O)cc3)cc2)c1NC(=O)O[C@H](C)c1ccccc1] |
| IT468 | 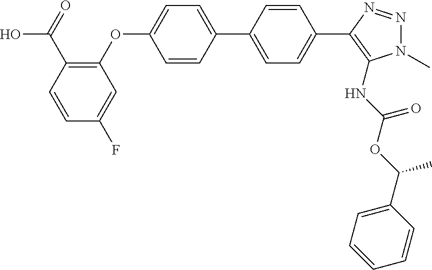 [SMILES: C[C@@H](OC(=O)Nc1c(-c2ccc(-c3ccc(Oc4cc(F)ccc4C(=O)O)cc3)cc2)nnn1C)c1ccccc1] |
| IT469 | 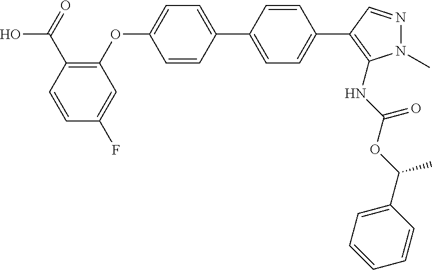 [SMILES: C[C@@H](OC(=O)Nc1c(-c2ccc(-c3ccc(Oc4cc(F)ccc4C(=O)O)cc3)cc2)cnn1C)c1ccccc1] |
| IT470 | 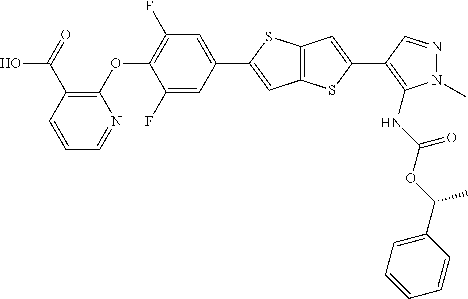 [SMILES: C[C@@H](OC(=O)Nc1c(-c2cc3sc(-c4cc(F)c(Oc5ncccc5C(=O)O)c(F)c4)cc3s2)cnn1C)c1ccccc1] |
| IT471 | 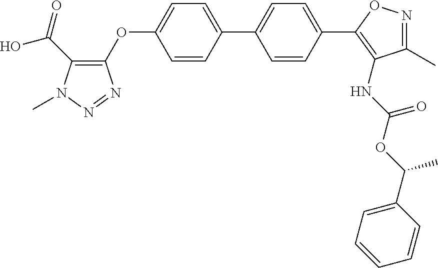 [SMILES: Cc1noc(-c2ccc(-c3ccc(Oc4nnn(C)c4C(=O)O)cc3)cc2)c1NC(=O)O[C@H](C)c1ccccc1] |
| IT472 | 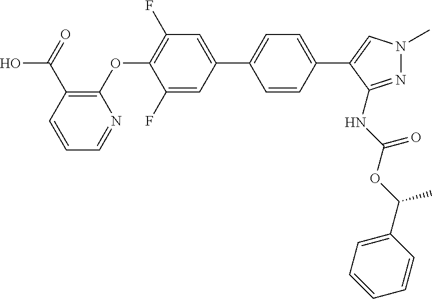 [SMILES: C[C@@H](OC(=O)Nc1nn(C)cc1-c1ccc(-c2cc(F)c(Oc3ncccc3C(=O)O)c(F)c2)cc1)c1ccccc1] |
| IT473 | 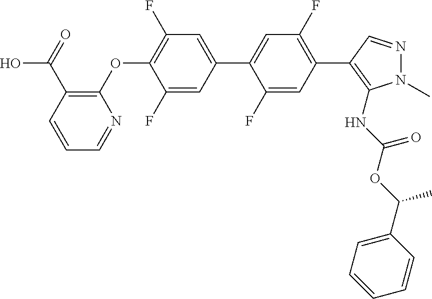 [SMILES: C[C@@H](OC(=O)Nc1c(-c2cc(F)c(-c3cc(F)c(Oc4ncccc4C(=O)O)c(F)c3)cc2F)cnn1C)c1ccccc1] |
| IT474 | 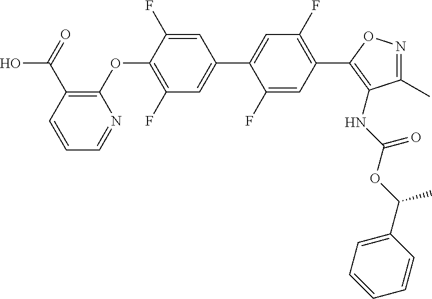 [SMILES: Cc1noc(-c2cc(F)c(-c3cc(F)c(Oc4ncccc4C(=O)O)c(F)c3)cc2F)c1NC(=O)O[C@H](C)c1ccccc1] |
| IT475 | 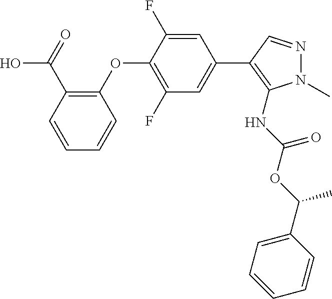 [SMILES: C[C@@H](OC(=O)Nc1c(-c2cc(F)c(Oc3ccccc3C(=O)O)c(F)c2)cnn1C)c1ccccc1] |
| IT476 | 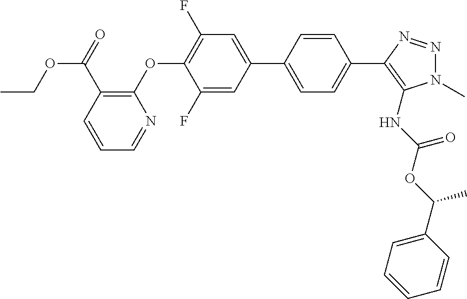 [SMILES: CCOC(=O)c1cccnc1Oc1c(F)cc(-c2ccc(-c3nnn(C)c3NC(=O)O[C@H](C)c3ccccc3)cc2)cc1F] |
| IT477 | 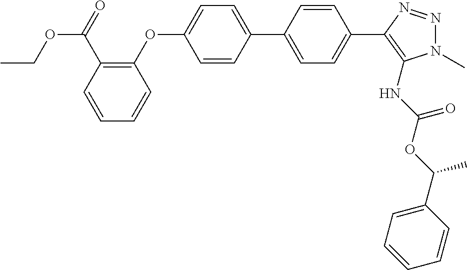 [SMILES: CCOC(=O)c1ccccc1Oc1ccc(-c2ccc(-c3nnn(C)c3NC(=O)O[C@H](C)c3ccccc3)cc2)cc1] |
| IT478 | 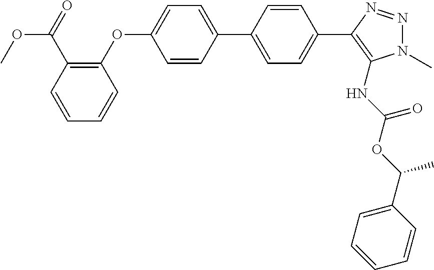 [SMILES: COC(=O)c1ccccc1Oc1ccc(-c2ccc(-c3nnn(C)c3NC(=O)O[C@H](C)c3ccccc3)cc2)cc1] |
| IT479 | 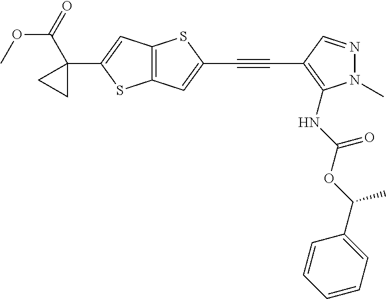 [SMILES: COC(=O)C1(c2cc3sc(C#Cc4cnn(C)c4NC(=O)O[C@H](C)c4ccccc4)cc3s2)CC1] |
| IT480 | 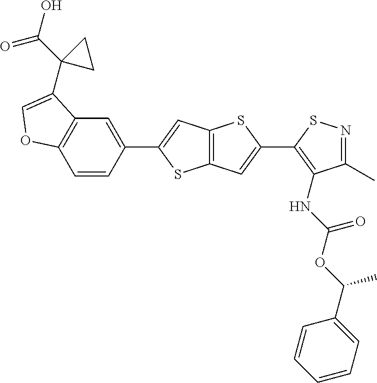 [SMILES: Cc1nsc(-c2cc3sc(-c4ccc5occ(C6(C(=O)O)CC6)c5c4)cc3s2)c1NC(=O)O[C@H](C)c1ccccc1] |
| IT481 | 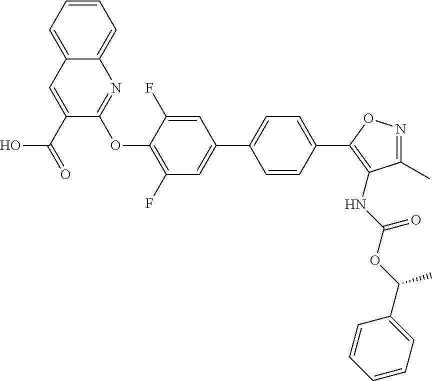 [SMILES: Cc1noc(-c2ccc(-c3cc(F)c(Oc4nc5ccccc5cc4C(=O)O)c(F)c3)cc2)c1NC(=O)O[C@H](C)c1ccccc1] |
| IT482 | 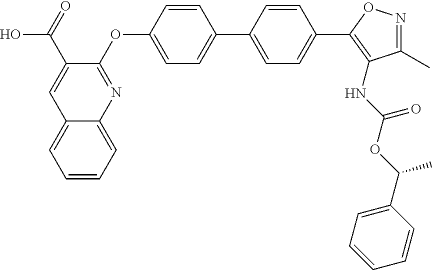 [SMILES: Cc1noc(-c2ccc(-c3ccc(Oc4nc5ccccc5cc4C(=O)O)cc3)cc2)c1NC(=O)O[C@H](C)c1ccccc1] |
| IT483 | 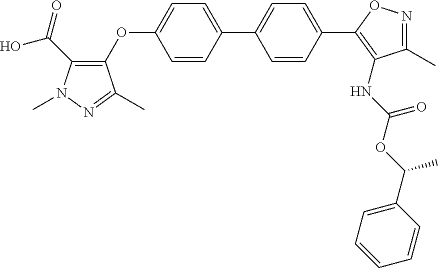 [SMILES: Cc1noc(-c2ccc(-c3ccc(Oc4c(C)nn(C)c4C(=O)O)cc3)cc2)c1NC(=O)O[C@H](C)c1ccccc1] |
| IT484 | 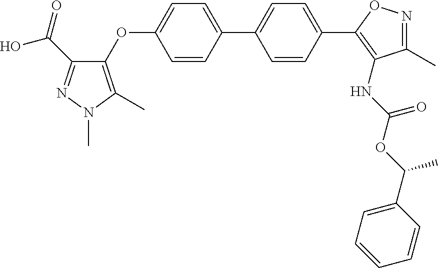 [SMILES: Cc1noc(-c2ccc(-c3ccc(Oc4c(C(=O)O)nn(C)c4C)cc3)cc2)c1NC(=O)O[C@H](C)c1ccccc1] |
| IT485 | 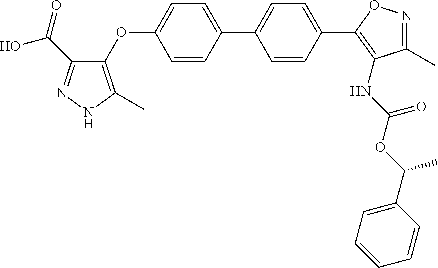 [SMILES: Cc1noc(-c2ccc(-c3ccc(Oc4c(C(=O)O)n[nH]c4C)cc3)cc2)c1NC(=O)O[C@H](C)c1ccccc1] |
| IT486 | 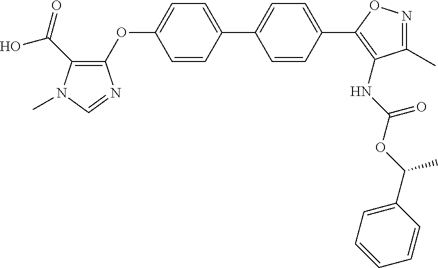 [SMILES: Cc1noc(-c2ccc(-c3ccc(Oc4ncn(C)c4C(=O)O)cc3)cc2)c1NC(=O)O[C@H](C)c1ccccc1] |
| IT487 | 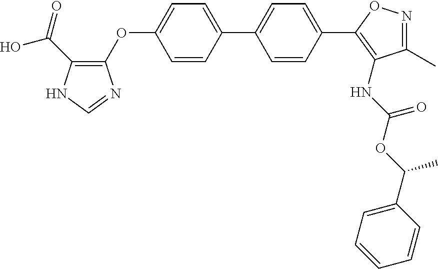 [SMILES: Cc1noc(-c2ccc(-c3ccc(Oc4nc[nH]c4C(=O)O)cc3)cc2)c1NC(=O)O[C@H](C)c1ccccc1] |
| IT488 | 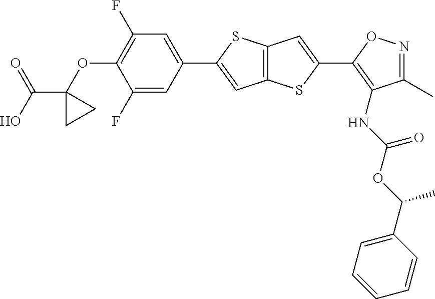 [SMILES: Cc1noc(-c2cc3sc(-c4cc(F)c(OC5(C(=O)O)CC5)c(F)c4)cc3s2)c1NC(=O)O[C@H](C)c1ccccc1] |
| IT489 | 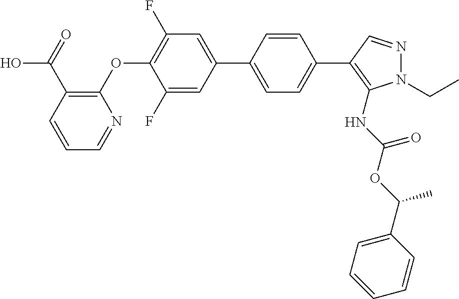 [SMILES: CCn1ncc(-c2ccc(-c3cc(F)c(Oc4ncccc4C(=O)O)c(F)c3)cc2)c1NC(=O)O[C@H](C)c1ccccc1] |
| IT490 | 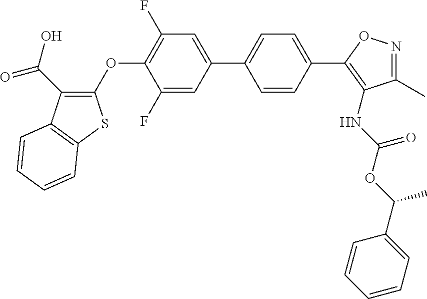 [SMILES: Cc1noc(-c2ccc(-c3cc(F)c(Oc4sc5ccccc5c4C(=O)O)c(F)c3)cc2)c1NC(=O)O[C@H](C)c1ccccc1] |
| IT491 | 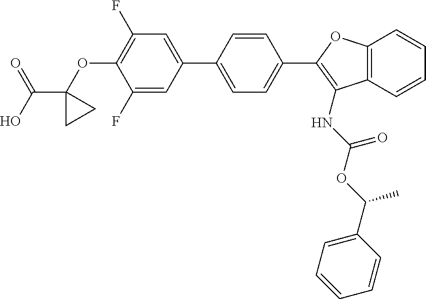 [SMILES: C[C@@H](OC(=O)Nc1c(-c2ccc(-c3cc(F)c(OC4(C(=O)O)CC4)c(F)c3)cc2)oc2ccccc12)c1ccccc1] |
| IT492 | 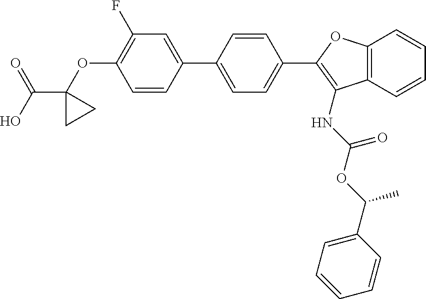 [SMILES: C[C@@H](OC(=O)Nc1c(-c2ccc(-c3ccc(OC4(C(=O)O)CC4)c(F)c3)cc2)oc2ccccc12)c1ccccc1] |
| IT493 | 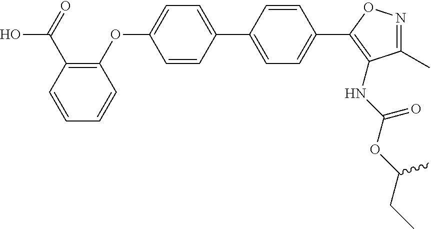 [SMILES: CCC(C)OC(=O)Nc1c(C)noc1-c1ccc(-c2ccc(Oc3ccccc3C(=O)O)cc2)cc1] |
| | Isomer 1 |
| IT494 | 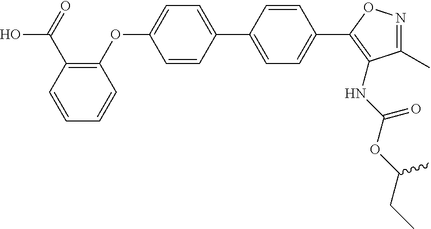 [SMILES: CCC(C)OC(=O)Nc1c(C)noc1-c1ccc(-c2ccc(Oc3ccccc3C(=O)O)cc2)cc1] |
| | Isomer 2 |
| IT495 | 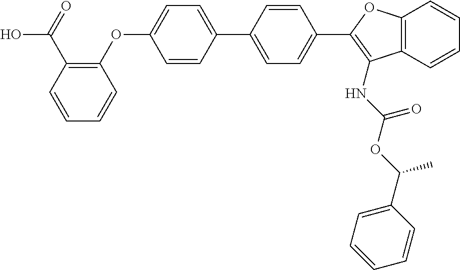 [SMILES: C[C@@H](OC(=O)Nc1c(-c2ccc(-c3ccc(Oc4ccccc4C(=O)O)cc3)cc2)oc2ccccc12)c1ccccc1] |
| | Isomer 2 |
| IT496 | 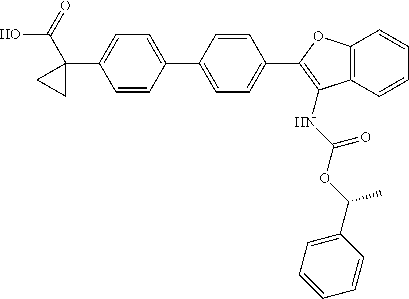 [SMILES: C[C@@H](OC(=O)Nc1c(-c2ccc(-c3ccc(C4(C(=O)O)CC4)cc3)cc2)oc2ccccc12)c1ccccc1] |
| IT497 | 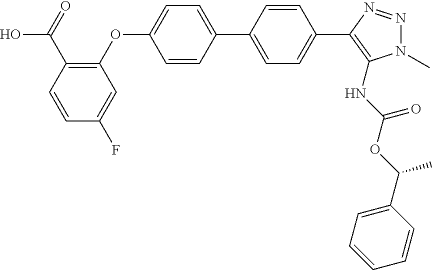 [SMILES: C[C@@H](OC(=O)Nc1c(-c2ccc(-c3ccc(Oc4cc(F)ccc4C(=O)O)cc3)cc2)nnn1C)c1ccccc1] |
| IT498 | 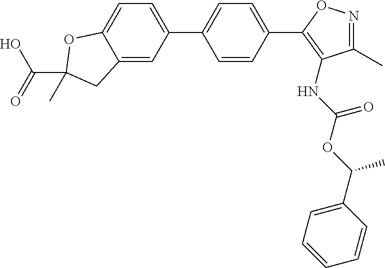 [SMILES: Cc1noc(-c2ccc(-c3ccc4c(c3)CC(C)(C(=O)O)O4)cc2)c1NC(=O)O[C@H](C)c1ccccc1] |
| IT499 | 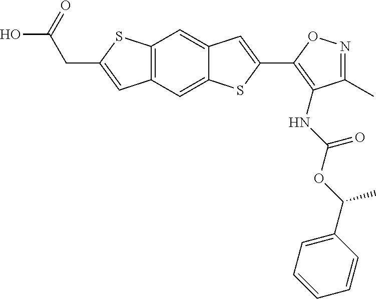 [SMILES: Cc1noc(-c2cc3cc4sc(CC(=O)O)cc4cc3s2)c1NC(=O)O[C@H](C)c1ccccc1] |
| IT500 | 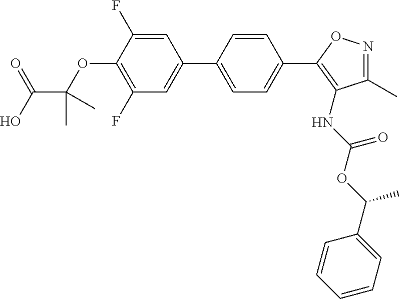 [SMILES: Cc1noc(-c2ccc(-c3cc(F)c(OC(C)(C)C(=O)O)c(F)c3)cc2)c1NC(=O)O[C@H](C)c1ccccc1] |
| IT501 | 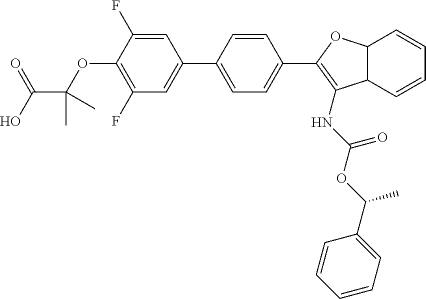 [SMILES: C[C@@H](OC(=O)NC1=C(c2ccc(-c3cc(F)c(OC(C)(C)C(=O)O)c(F)c3)cc2)OC2C=CC=CC12)c1ccccc1] |
| IT502 | 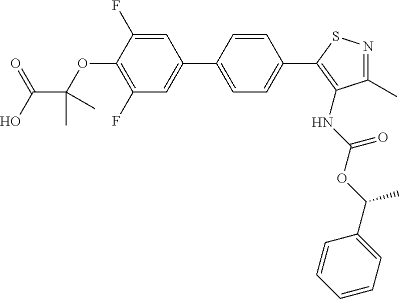 [SMILES: Cc1nsc(-c2ccc(-c3cc(F)c(OC(C)(C)C(=O)O)c(F)c3)cc2)c1NC(=O)O[C@H](C)c1ccccc1] |
| IT503 | 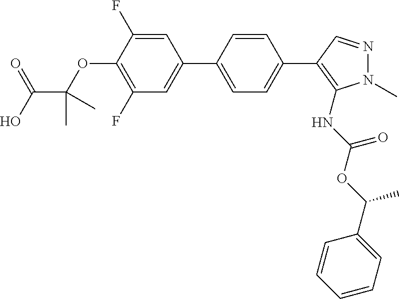 [SMILES: C[C@@H](OC(=O)Nc1c(-c2ccc(-c3cc(F)c(OC(C)(C)C(=O)O)c(F)c3)cc2)cnn1C)c1ccccc1] |
| IT504 | 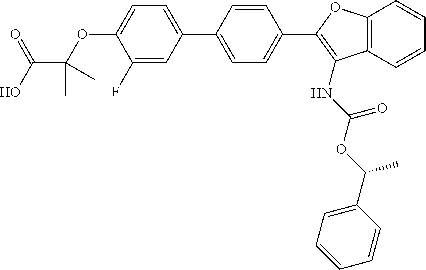 [SMILES: C[C@@H](OC(=O)Nc1c(-c2ccc(-c3ccc(OC(C)(C)C(=O)O)c(F)c3)cc2)oc2ccccc12)c1ccccc1] |
| IT505 | 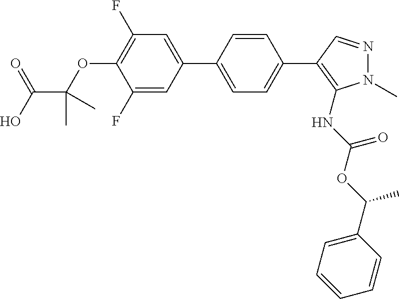 [SMILES: C[C@@H](OC(=O)Nc1c(-c2ccc(-c3cc(F)c(OC(C)(C)C(=O)O)c(F)c3)cc2)cnn1C)c1ccccc1] |
| IT506 | 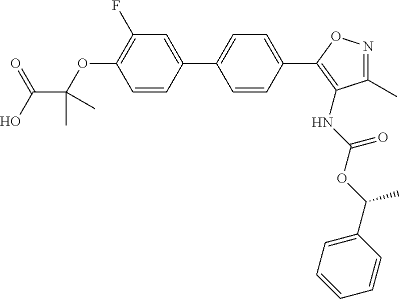 [SMILES: Cc1noc(-c2ccc(-c3ccc(OC(C)(C)C(=O)O)c(F)c3)cc2)c1NC(=O)O[C@H](C)c1ccccc1] |
| IT507 | 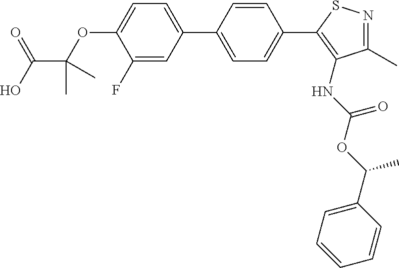 [SMILES: Cc1nsc(-c2ccc(-c3ccc(OC(C)(C)C(=O)O)c(F)c3)cc2)c1NC(=O)O[C@H](C)c1ccccc1] |
| IT508 | 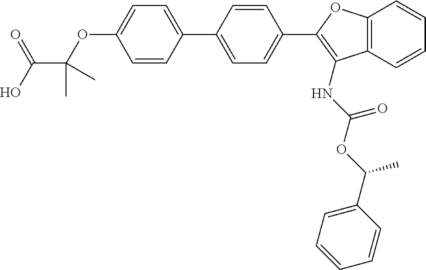 [SMILES: C[C@@H](OC(=O)Nc1c(-c2ccc(-c3ccc(OC(C)(C)C(=O)O)cc3)cc2)oc2ccccc12)c1ccccc1] |
| IT509 | 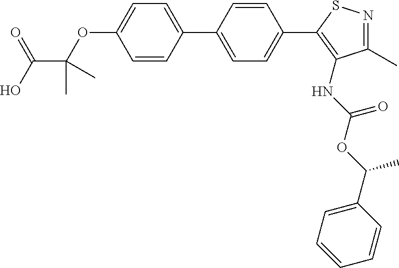 [SMILES: Cc1nsc(-c2ccc(-c3ccc(OC(C)(C)C(=O)O)cc3)cc2)c1NC(=O)O[C@H](C)c1ccccc1] |
| IT510 | 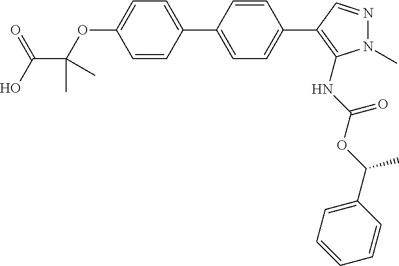 [SMILES: C[C@@H](OC(=O)Nc1c(-c2ccc(-c3ccc(OC(C)(C)C(=O)O)cc3)cc2)cnn1C)c1ccccc1] |
| IT511 | 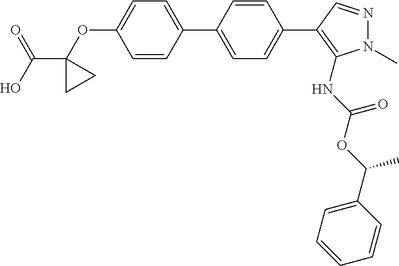 [SMILES: C[C@@H](OC(=O)Nc1c(-c2ccc(-c3ccc(OC4(C(=O)O)CC4)cc3)cc2)cnn1C)c1ccccc1] |
| IT512 | 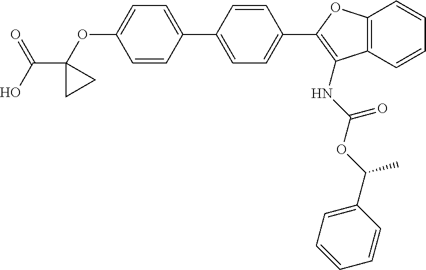 [SMILES: C[C@@H](OC(=O)Nc1c(-c2ccc(-c3ccc(OC4(C(=O)O)CC4)cc3)cc2)oc2ccccc12)c1ccccc1] |
| IT513 | 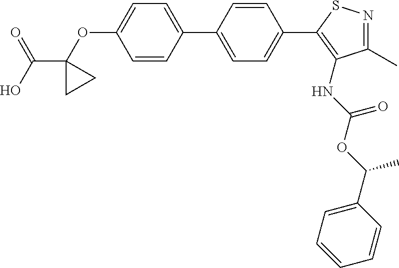 [SMILES: Cc1nsc(-c2ccc(-c3ccc(OC4(C(=O)O)CC4)cc3)cc2)c1NC(=O)O[C@H](C)c1ccccc1] |
| IT514 | 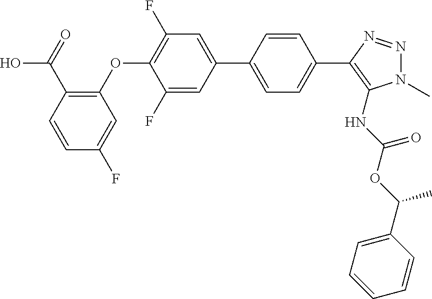 [SMILES: C[C@@H](OC(=O)Nc1c(-c2ccc(-c3cc(F)c(Oc4cc(F)ccc4C(=O)O)c(F)c3)cc2)nnn1C)c1ccccc1] |

Diseases, Disorders and Conditions Associated with LPA Activity

The compounds of preferred embodiments inhibit the physiological activity of LPA. As such the compounds of preferred embodiments are useful as agents for the treatment or prevention of diseases in which inhibition of the physiological activity of LPA is desirable, such as in the treatment of diseases in which an LPA receptor participates, or is involved in the etiology or pathology of the disease, or is otherwise associated with at least one symptom of the disease. The compounds of preferred embodiments can be employed for the treatment or prevention of side effects, complications, or adverse events associated with the use of a conventional therapeutic agent or therapeutic action (e.g., surgery, etc.) used in treating a disease or condition in which inhibition of LPA physiological activity is desirable. The compounds of preferred embodiments are antagonists of at least one of the LPA receptors, e.g., LPA₁, LPA₂, LPA₃, LPA₄, LPA₅, and/or LPA₆. Certain of the compounds of preferred embodiments are selective antagonists for one or more of the LPA receptors relative to the other LPA receptors.

The compounds of preferred embodiments are used in the treatment of diseases, disorders, or conditions in which activation of at least one LPA receptor by LPA contributes to the symptomology or progression of the disease, disorder, or condition. The compounds of preferred embodiments are antagonists of LPA receptor(s). Diseases, disorders, or conditions that the compounds of preferred embodiments can be used to treat include, but are not limited to, fibrosis, cancer, or respiratory disorders. For examples, the fibrosis can include pulmonary fibrosis, dermal fibrosis, kidney fibrosis, or liver fibrosis. In one embodiment, the fibrosis is idiopathic pulmonary fibrosis.

The terms “fibrosis” or “fibrosing disorder,” as used herein, are broad terms and refer without limitation to conditions that are associated with the abnormal accumulation of cells and/or fibronectin and/or collagen and/or increased fibroblast recruitment and include but are not limited to fibrosis of individual organs or tissues such as the lung. Exemplary diseases, disorders, or conditions that involve fibrosis include, but are not limited to, idiopathic pulmonary fibrosis.

LPA and LPA₁ play key pathogenic roles in pulmonary fibrosis. Fibroblast chemoattractant activity plays a role in the lungs in patients with pulmonary fibrosis. Profibrotic effects of LPA₁-receptor stimulation is explained by LPA₁-receptor-mediated vascular leakage and increased fibroblast recruitment, both profibrotic events. The LPA-LPA₁ pathway has a role in mediating fibroblast migration and vascular leakage in IPF. The end result is the aberrant healing process that characterizes this fibrotic condition. The LPA-LPA₂ pathway contributes to the activation of the TGF-β pathway in pulmonary fibrosis. Compounds that inhibit LPA₂ may show efficacy in the treatment of lung fibrosis. Compounds that inhibit both LPA₁ and LPA₂ may show improved efficacy in the treatment of lung fibrosis compared to compounds which inhibit only LPA₁ or LPA₂.

Some embodiments described herein relate to a method of treating a fibrotic condition, which can include administering a therapeutically effective amount of a compound disclosed herein, or a pharmaceutically acceptable salt thereof, to a subject. The methods include identifying a subject at risk for or having a fibrotic condition and administering a compound to the subject in an effective amount for therapeutic treatment or prophylactic treatment of the fibrotic condition.

A “fibrotic condition,” “fibroproliferative condition,” “fibrotic disease,” “fibroproliferative disease,” “fibrotic disorder,” and “fibroproliferative disorder” are used interchangeably to refer to a condition, disease or disorder that is characterized by dysregulated proliferation or activity of fibroblasts and/or abnormal accumulation of fibronectin and/or pathologic or excessive accumulation of collagenous tissue. Typically, any such disease, disorder or condition is amenable to treatment by administration of a compound having anti-fibrotic activity. Fibrotic disorders include, but are not limited to, pulmonary fibrosis, including idiopathic pulmonary fibrosis (IPF) and pulmonary fibrosis from a known etiology, dermal fibrosis, pancreatic fibrosis, liver fibrosis (e.g., hepatic fibrosis associated with chronic active hepatitis), and renal fibrosis.

In some embodiments, the subject is a human.

The terms “therapeutically effective amount,” as used herein, refer to an amount of a compound sufficient to cure, ameliorate, slow progression of, prevent, or reduce the likelihood of onset of the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. However, pharmaceutical compositions that exhibit narrow therapeutic indices are also within the scope of the invention. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In one aspect, treating a condition described herein results in an increase in average survival time of a population of treated subjects in comparison to a population of untreated subjects. Preferably, the average survival time is increased by more than about 30 days; more preferably, by more than about 60 days; more preferably, by more than about 90 days; and even more preferably by more than about 120 days. An increase in survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In another aspect, treating a condition described herein results in a decrease in the mortality rate of a population of treated subjects in comparison to a population of subjects receiving carrier alone. In another aspect, treating a condition described herein results in a decrease in the mortality rate of a population of treated subjects in comparison to an untreated population. In a further aspect, treating a condition described herein results a decrease in the mortality rate of a population of treated subjects in comparison to a population receiving monotherapy with a drug that is not a compound of the embodiments, or a pharmaceutically acceptable salt, metabolite, analog or derivative thereof. Preferably, the mortality rate is decreased by more than about 2%; more preferably, by more than about 5%; more preferably, by more than about 10%; and most preferably, by more than about 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease related deaths per unit time following completion of a first round of treatment with an active compound.

In another aspect, treating a condition described herein results in a reduction in the rate of cellular proliferation. Preferably, after treatment, the rate of cellular proliferation is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating a condition described herein results in a reduction in the proportion of proliferating cells. Preferably, after treatment, the proportion of proliferating cells is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The proportion of proliferating cells may be measured by any reproducible means of measurement. In a preferred aspect, the proportion of proliferating cells is measured, for example, by quantifying the number of dividing cells relative to the number of nondividing cells in a tissue sample. In another preferred aspect, the proportion of proliferating cells is equivalent to the mitotic index.

In another aspect, treating a condition described herein results in a decrease in size of an area or zone of cellular proliferation. Preferably, after treatment, size of an area or zone of cellular proliferation is reduced by at least 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 60%; and most preferably, reduced by at least about 75%. Size of an area or zone of cellular proliferation may be measured by any reproducible means of measurement. In a preferred aspect, size of an area or zone of cellular proliferation may be measured as a diameter or width of an area or zone of cellular proliferation.

The methods described herein may include identifying a subject in need of treatment. In a preferred embodiment, the methods include identifying a mammal in need of treatment. In a highly preferred embodiment, the methods include identifying a human in need of treatment. Identifying a subject in need of treatment may be accomplished by any means that indicates a subject who may benefit from treatment. For example, identifying a subject in need of treatment may occur by clinical diagnosis, laboratory testing, or any other means known to one of skill in the art, including any combination of means for identification.

As described elsewhere herein, the compounds described herein may be formulated in pharmaceutical compositions, if desired, and can be administered by any route that permits treatment of the disease or condition. A preferred route of administration is oral administration. Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,”it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

### Pharmaceutical Compositions/Formulations, Routes of Administration, and Methods of Treatment

In some embodiments, the compounds described herein are prepared into pharmaceutical compositions. Pharmaceutical compositions suitable for administration to a patient in need thereof can be prepared using techniques known in the art. Pharmaceutically acceptable inactive ingredients that facilitate processing of the active compounds into pharmaceutical compositions can also be employed. Once a route of administration chosen, a pharmaceutical composition can be developed. Suitable pharmaceutical compositions include those described, e.g., in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), the contents of which are hereby expressly incorporated by reference herein.

Pharmaceutical compositions suitable for use in the methods of preferred embodiments include a mixture of one or more compounds of a preferred embodiment with other chemical components (e.g., pharmaceutically acceptable inactive or active ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to a patient in need thereof.

The pharmaceutical compositions of preferred embodiments can be systemically and/or locally administrable to a patent in need thereof in a variety of ways and by multiple administration routes, including, but not limited to, oral, parenteral (e.g., intravenous, subcutaneous, intramuscular, intramedullary injections, intrathecal, direct intraventricular, intraperitoneal, intralymphatic, intranasal injections), inhalation, injection (e.g., intramuscular, subcutaneous, or intravenous), rectal (e.g., enemas, rectal gels, rectal foams, rectal aerosols, suppositories, jelly suppositories, or retention enemas), intranasal, buccal, topical or transdermal administration routes. Such pharmaceutical compositions can be in a form of aqueous liquid dispersions, aqueous oral dispersions, emulsions, solutions, elixirs, gels, syrups, self-emulsifying dispersions, solid solutions, liposomal dispersions, aerosols, mists, solid dosage forms, powders, nasal sprays, nasal mists, eye drops immediate release formulations, controlled release formulations, fast melt formulations, tablets, lozenge, capsules, pills, delayed release formulations, extended release formulations, pulsatile release formulations, multiparticulate formulations, and mixed immediate and controlled release formulations. Topically administrable compositions include solutions, suspensions, lotions, gels, pastes, shampoos, scrubs, rubs, smears, medicated sticks, medicated bandages, balms, creams, or ointments.

In some embodiments, a compound described herein can be prepared in inhalable formulations for administration via an atomizer. An atomizer allows a stream of air to move at a high velocity over the tip of a tube dipped in a solution. The pressure at the tip of the tube is lowered and the solution is drawn into the air flow. The solution disperses into a fine spray or droplets that are carried into the inhaled stream of air.

In some embodiments the inhalable solution formulations described herein are administered with a nebulizer that is placed in the mouth. The spray, mist or fine droplets produced by atomizers or nebulizers allow the compound described herein to reach the bronchioles in the lungs. Various nebulizers suitable for this use include jet nebulizers, ultrasonic nebulizers, and vibrating mesh nebulizers. A jet nebulizer utilizes air pressure breakage of an aqueous solution into aerosol droplets. An ultrasonic nebulizer utilizes shearing of the aqueous solution by a piezoelectric crystal. Vibrating mesh nebulizers rely upon either piezoelectric or mechanical pulses to generate respirable liquid droplets. A vibrating mesh nebulizer consists of a liquid storage container in fluid contact with a diaphragm and inhalation and exhalation valves. Commercial examples of nebulizers that can be used include Respirgard II®, Aeroneb®, Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LC-Plus®, PARI LC-Star®, and e-Flow^{7m} produced by PARI, GmbH.

By non-limiting example, a compound disclosed herein is placed in a liquid nebulization inhaler and prepared in dosages to deliver from about 7 to about 700 mg from a dosing solution of about 1 to about 5 ml, preferably from about 14 to about 350 mg in about 1 to about 5 ml, and most preferably from about 28 to about 280 mg in about 1 to about 5 ml with mass median aerodynamic diameter (MMAD) particles sizes between about 2 to about 5 um being produced.

By non-limiting example, a nebulized compound disclosed herein may be administered in the prescribed respirable delivered dose in less than about 20 min, preferably less than about 10 min, more preferably less than about 7 min, more preferably less than about 5 min, more preferably less than about 3 min, and in some cases most preferable if less than about 2 min.

In some embodiments, the inhalable formulations described herein comprise a propellant and are pressure packaged for administration of a compound described herein using pressurized aerosols. In the case of a pressurized aerosol, the dosage unit may be determined by providing a valve to deliver a metered amount.

In some embodiments, the inhalable formulations described herein are administered with a metered dose spray bottle that delivers a specific volume of a solution, suspension, emulsion or colloidal dispersion for inhalation.

In some embodiments, dry powder inhalable formulations are administered with an insufflator. An insufflator consists of a rubber bulb connected to a container and a delivery pipe. As the bulb is squeezed, air is blown into the container and causes the powder to move. The particles are carried out via the delivery tube and are inhaled.

In some embodiments, dry powder inhalable formulations are administered with a puffer. The dry powder is placed in the puffer and the puffer is squeezed. A portion of the powder is ejected from the spout into the air and is inhaled. Capsules and cartridges of, such as, by way of example only, gelatin for use in an inhaler or insufflator may be formulated containing a dry powder formulation.

In some embodiments, a propellant driven inhaler (pMDI) releases a metered dose of a compound described herein upon each actuation. In such applications, the compound can be formulated as a suspension or solution of a drug substance in a suitable propellant such as a halogenated hydrocarbon. The propellants for use with the MDIs may be any propellants known in the art. Examples of propellants include chlorofluorocarbons (CFCs) such as dichlorodifluoromethane, trichlorofluorometbane, and dichlorotetrafluoroethane; hydrofluoroalkanes (HFAs); and carbon dioxide.

Excipients

In some embodiments, the compounds described herein are administered via an inhalable formulation comprising one or more excipients. Alternatively, the compounds may be administered without excipients.

The excipients described herein include, but not limited to, pharmaceutical grades of carbohydrates (monosaccharides, disaccharides, polysaccharides such as hyaluronic acid, heparin/heparan sulfate, dermatan sulfate, chondroitin sulfate, keratin sulfate, alginic acid and salts thereof, and cellulose; oligosaccharides, polyols, and combinations and derivatives thereof), organic and inorganic salts, polymers including natural biodegradable protein polymers, natural biodegradable polysaccharide polymers, synthetic polymers and synthetic biodegradable polymers, amino acids, phospholipids, wetting agents, emulsifiers, surfactants, poloxamers, pluronics, and ion exchange resins, and combinations thereof.

In some embodiments, the compound described herein is in an inhalable formulation for delivery to the lungs that comprises one or more pH adjusting agents. Examples of pH adjusting agents or buffering agents, include, but are not limited to acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, the compound described herein is in an inhalable formulation for delivery to the lungs of a mammal that comprises one or more tonicity agents. Tonicity agents are used to adjust the composition of the formulation to the desired isotonic range. Tonicity agents include one or more salts in an amount required to bring osmolality of the composition into an acceptable range. Non-limiting examples of these salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. Other exemplary tonicity agents include mannitol, dextrose,

In some embodiments, the compound described herein is in an inhalable formulation for delivery to the lungs of a mammal that comprises one or more preservatives to inhibit microbial activity. Non-limiting examples of suitable preservatives include benzoic acid, boric acid, p-hydroxybenzoates, alcohols, mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, the formulations described herein optionally include one or more stabilizers (e.g., antioxidants) to enhance chemical stability where required. Non-limiting examples of suitable antioxidants include, ascorbic acid, methionine, sodium thiosulfate and sodium metabisulfite. In some embodiments, antioxidants are selected from metal chelating agents, thiol containing compounds and other general stabilizing agents.

In some embodiments, the compound described herein is in an inhalable formulation for delivery to the lungs that comprises one or more propellants. Non-limiting exemplary propellants include one or mixture of chlorofluorocarbons, such as dichlorodifiuoromethane, trichlorofiuoromethane, dichlorotetrafluoroethane or the like, as well as hydrofluorocarbons, such as 1,1,1,2-tetrafluoroethane (HFC-134a) and 1,1,1,2,3,3,3-heptafluoropropane (HFC-227) or the like, carbon dioxide or other suitable gas. In certain embodiments, the propellants are used with a co-solvent. Non-limiting exemplary co-solvents include alcohols such as ethyl alcohol, isopropyl alcohol, propylene glycol, hydrocarbons such as propane, butane, isobutane, pentane, isopentane, neopentane, and other propellants such as those commonly referred to as Propellants 11, 12, 114, 113, 142b, 152a 124, and dimethyl ether.

In some embodiments, the compound described herein is in an inhalable formulation for delivery to the lungs that comprises one or more surfactants. Non-limiting examples of surfactants for inhalable formulations include and are not limited to oils derived from natural sources, such as, corn oil, olive oil, cotton seed oil and sunflower seed oil; sorbitan esters, such as Sorbitan trioleate available under the trade name Span 85, Sorbitan mono-oleate available under the trade name Span 80, Sorbitan monolaurate available under the trade name Span 20, Polyoxyethylene (20) sorbitan monolaurate available under the trade name Tween 20, Polyoxyethylene (20) sorbitan mono-oleate available under the trade name Tween 80; lecithins derived from natural sources such as those available under the trade name Epikuron particularly Epikuron 200. Oleyl polyoxyethylene (2) ether available under the trade name Brij 92, Stearyl polyoxyethylene (2) available under the trade name Brij 72, Lauryl polyoxyethylene (4) ether available under the trade name Brij 30, Oleyl polyoxyethylene (2) ether available under the trade name Genapol 0-020, Block copolymers of oxyethylene and oxypropylene available under the trade name Synperonic, Oleic acid, Synthetic lecithin, Diethylene glycol dioleate, Tetrahydrofurfuryl oleate, Ethyl oleate, Isopropyl myristate, Glyceryl trioleate, Glyceryl monolaurate, Glyceryl mono-oleate, Glyceryl monostearate, Glyceryl monoricinoleate, Cetyl alcohol, Stearyl alcohol, Polyethylene glycol 400, and Cetyl pyridinium chloride.

In some embodiments, the solution, emulsion, suspension and/or colloidal dispersion formulations also include inert diluents commonly used in the art, such as water or other solvents, solubilizing agents, and/or emulsifiers. Non-limiting exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the inhalable formulations described herein are stable (e.g., with respect to pH, active ingredient) over a period of any of at least about 1 day, at least about 2 days, at least about 3 days, at least about 4 days, at least about 5 days, at least about 6 days, at least about 1 week, at least about 2 weeks, at least about 4 weeks, at least about 6 weeks, at least about 8 weeks, at least about 4 months, at least about 5 months, at least about 6 months, or greater than 6 months.

In certain embodiments, the inhalable formulations described herein are designed for minimal pulmonary toxicity, irritation and/or allergic challenge to pulmonary tissues and include, for example, low amounts of excipients such as surfactants, preservatives and/or co-solvents.

The compounds of preferred embodiments and pharmaceutical compositions comprising the same can be used for treating, preventing, reversing, halting or slowing the progression of LPA-dependent or LPA-mediated diseases or conditions once it becomes clinically evident, or treating the symptoms associated with or related to LPA-dependent or LPA-mediated diseases or conditions, by administering the compound to a subject in need thereof, e.g., a subject that has a LPA-dependent or LPA-mediated disease or condition at the time of administration, or is at risk of developing a LPA-dependent or LPA-mediated disease or condition.

Also provided are methods that include the diagnosis or determination of whether or not a patient is suffering from a LPA-dependent or LPA-mediated disease or condition by administering to the subject a therapeutically effective amount of a compound of a preferred embodiment and determining whether or not the patient responds to the treatment.

The pharmaceutical compositions can be administered continuously or intermittently, e.g., in single administrations of an effective amount of the compound, or administrations twice, three times, or four times or more over the span of one day. The pharmaceutical compositions can be administered over a single day or multiple days, with a time between administrations of, e.g., 4, 5, 6, 7, 8, 9, 10, 11, 12 or 24 hours. For example, the compound of preferred embodiments can be administered continuously or intermittently as in a single dose; or in multiple doses with a dose administered every 6 hours, or 8 hours, or 12 hours, or 24 hours. Also contemplated are administration methods including a drug holiday, wherein the administration of the compound is temporarily suspended or the dose of the compound being administered is temporarily reduced; at the end of the drug holiday, dosing of the compound is resumed. The length of the drug holiday varies from 2 days, or 1 month, or two months, or 3 months, or 6 months, or 9 months, to 1 year or more. The pharmaceutical composition can be administered therapeutically or prophylactically for a fixed period of time indefinitely.

The compounds of preferred embodiments can be used in the preparation of medicaments for the treatment of LPA-dependent or LPA-mediated diseases or conditions. Treatment involves administration of pharmaceutical compositions that include at least one compound of preferred embodiments or a pharmaceutically acceptable salt, active metabolite, prodrug, or solvate thereof, in a therapeutically effective amount, to said patient. The compounds of preferred embodiments can be administered for prophylactic and/or therapeutic treatment. In certain therapeutic applications, the compositions are administered to a patient already suffering from a disease or condition, in an amount sufficient to cure or at least partially mitigate at least one of the symptoms of the disease or condition. Amounts effective for this use depend on the severity and course of the disease or condition, previous therapy, the patient's health status, weight, and response to the drugs, and the judgment of the treating physician. Therapeutically effective amounts can be determined by methods including, but not limited to, a dose escalation clinical trial. In prophylactic applications, the compounds of preferred embodiments are administered to a patient susceptible to or otherwise at risk of a particular disease, disorder, or condition. The dose of drug being administered may be temporarily reduced or temporarily suspended for a certain length of time (i.e., a “drug holiday”).

Doses employed for adult human treatment are typically in the range of 0.01 mg to 5000 mg per day, or from about 1 mg to about 1000 mg per day. The desired dose can be provided in a single dose or in divided doses.

In certain embodiments, patients in need of treatment can be identified by screening for LPA receptor gene SNPs. Patients can be further selected based on increased LPA receptor expression in the tissue of interest. LPA receptor expression are determined by methods including, but not limited to, northern blotting, western blotting, quantitative PCR (qPCR), flow cytometry, autoradiography (using a small molecule radioligand or PET ligand). In some embodiments, patients are selected based on the concentration of serum or tissue LPA measured by mass spectrometry. In some embodiments, patients are selected based on a combination of the above markers (increased LPA concentrations and increased LPA receptor expression).

In certain embodiments, the compounds of preferred embodiments are administered with another therapeutic treatment or another therapeutic agent, e.g., a second therapeutic agent that modulates different aspects of the disease, disorder or condition being treated, thereby providing a greater overall benefit than administration of either therapeutic agent alone. For combination therapies, the dosages of the co-administered compounds vary depending on the type or specific drug employed, on the disease or condition being treated, and other factors. When co-administered with one or more other therapeutic agents, the compounds of preferred embodiments can be administered either simultaneously with the one or more other therapeutic agents, or sequentially, and can be present in the same unit dosage form or in different unit dosage forms. If administration is simultaneous, the multiple therapeutic agents are, by way of example only, provided in a single, unified form, or in multiple forms. In the treatment of cancer, it is advantageous to administer a compound of a preferred embodiment in combination with one or more anti-cancer agents and/or radiation therapy. In the treatment of fibrosis, it is advantageous to administer a compound of a preferred embodiment in combination with one or more immunosuppressant and/or with corticosteroids. In treating LPA-dependent or LPA-mediated conditions or diseases, such as the therapy of respiratory disorders (e.g., pulmonary fibrosis, asthma, COPD, rhinitis), it is advantageous to administer a compound of a preferred embodiment in combination with one or more agents used in the treatment of respiratory conditions, e.g., anti-inflammatory agents or inhaled corticosteroids.

### Synthesis

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Some embodiments described herein relate to method of preparing compounds of formula (VIIb), comprising conducting a palladium catalyzed cross-coupling reaction between a compound of formula (VII-1) and a compound of formula (VII-3) as shown in Scheme 1 below. Alternatively, compounds of formula (VIIb) can be prepared by conducting a palladium catalyzed cross-coupling reaction between a compound of formula (VII-2) and a compound of formula (VII-4) as shown in Scheme 2 below:

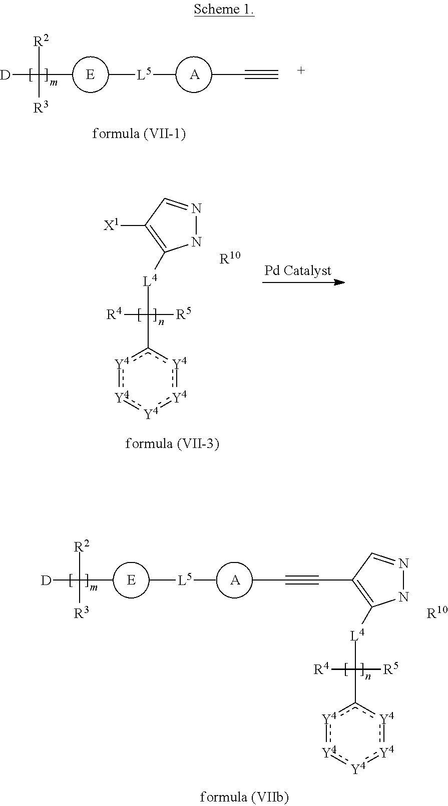

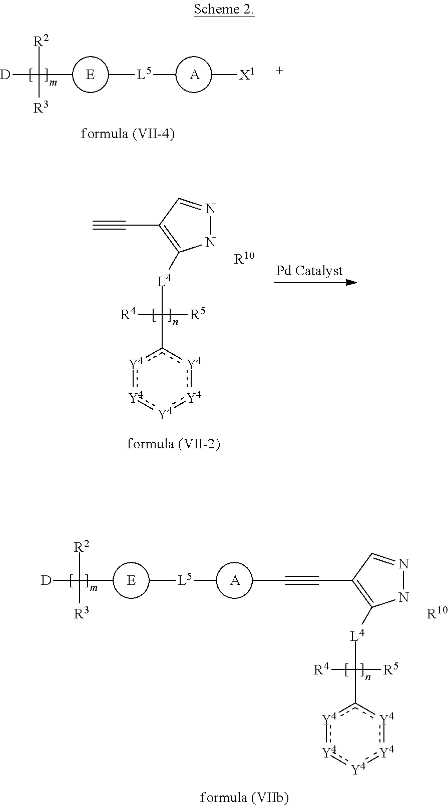

wherein X¹ is a halogen selected from Br or I;

A is a ring system selected from the group consisting of

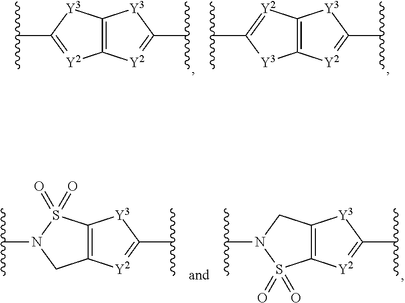

wherein A is optionally substituted;

D is selected from —OH,

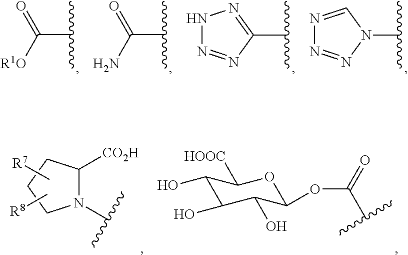

### —NR¹³SOₚR¹⁴, —C(O)—NR¹³SOₚR¹⁴,

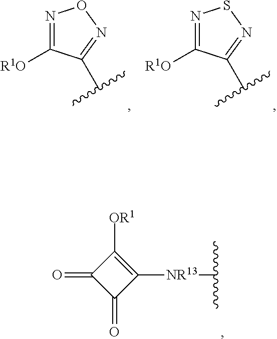

—SOₚR¹⁵, —SOₚNR¹⁶R¹⁷, or carboxylic acid isosteres;

E is absent or selected from 6-10 membered arylene, 3-11 membered carbocyclyl, 3-11 membered heterocyclyl or 5 to 10 membered heteroarylene, wherein E is optionally substituted;

L⁵ is selected from a single bond, a —CH═CH— linker, a —C≡C— linker,

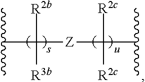

or a 4-7 membered heterocyclyl;

Z is selected from C(O), O, S, S(O)₂, NR^{6a}, C(O)NR^{6b}, or S(O)₂NR^{6c};

R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;

R² and R³ are each independently selected from hydrogen, alkyl, halogen, haloalkyl, cycloalkyl, heterocyclyl, aryl, or heteroaryl, wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;

or R² and R³ are joined together with the atom to which they are attached to form an optionally substituted cycloalkyl or an optionally substituted heterocyclyl;

or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;

each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;

each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;

each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;

each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;

each R¹⁰ is independently selected from hydrogen; alkyl optionally substituted with one or more substituents selected from the group consisting of halogen, hydroxy and alkoxy; halogen; aryl; C₃₋₆ cycloalkyl; or cyano;

each R¹³ and R¹⁴ is independently selected from hydrogen, alkyl, haloalkyl, aryl, or C₃₋₆ cycloalkyl;

R¹⁵ is selected from hydrogen, alkyl, haloalkyl, (carbocyclyl)alkyl, aryl, or C₃₋₆ cycloalkyl;

each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;

L⁴ is selected from

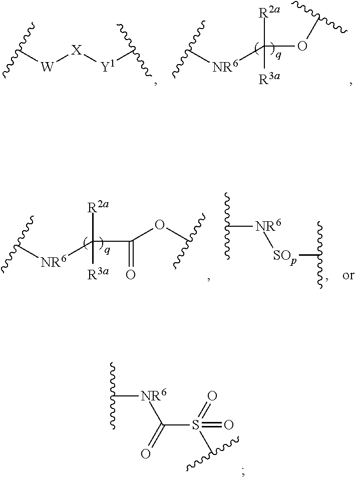

each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;

W is selected from C(R⁶)₂, NR⁶, or O;

X is selected from —C(O) or S(O)ₚ;

Y¹ is selected from C(R⁶)₂, NR⁶, or O;

each Y² is independently selected from —CH═ or N;

each Y³ is independently selected from C(R⁶)₂, NR⁶, O or S;

each Y⁴ is independently absent, CR⁹, C(R⁹)₂, N, or NH, provided that only one Y⁴ can be absent;

m is an integer from 0-3;

n is an integer from 0-3;

p is an integer from 1-2;

q is an integer from 1-6;

s and u are independently an integer from 0 to 6; and

[symbol] represents a single or double bond.

In some embodiments, A can be optionally substituted with one or more substituents selected from the group consisting of alkyl, amino, haloalkyl, halogen, hydroxy, alkoxy, haloalkoxy, cyano and or oxo; and E can be absent or optionally substituted with one or more substituents selected from the group consisting of alkyl, amino, haloalkyl, halogen, hydroxy, alkoxy, haloalkoxy, cyano and or oxo. In some embodiments, each A and E can be optionally substituted with one or more sulfonyl groups, for example, methanesulfonyl.

In some embodiments, A is unsubstituted or substituted with one or more substituents selected from alkyl, haloalkyl, halogen, hydroxy, alkoxy, haloalkoxy, cyano, or oxo;

D is selected from —OH,

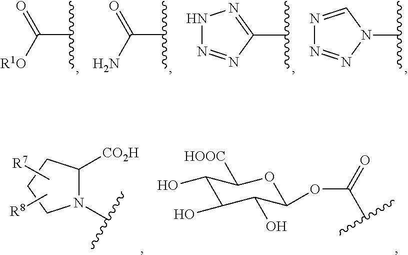

### —NR¹³SOₚR¹⁴, —C(O)—NR¹³SOₚR¹⁴,

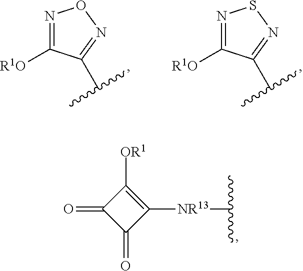

or carboxylic acid isosteres; E is absent; L⁵ is selected from a single bond, a —CH₂O— linker, a —CH═CH— linker, a —C≡C— linker,

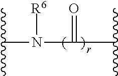

or a 4-7 membered heterocyclyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; and r is an integer of 0 or 1.

In some embodiments, the compound of formula (VII-3) is also represented by formula (VII-3A):

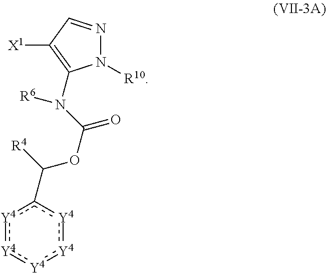

In some embodiments, the compound of formula (VII-4) is also represented by formula (VII-4A):

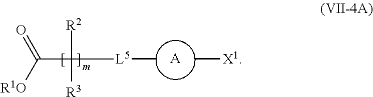

In some embodiments, A is selected from

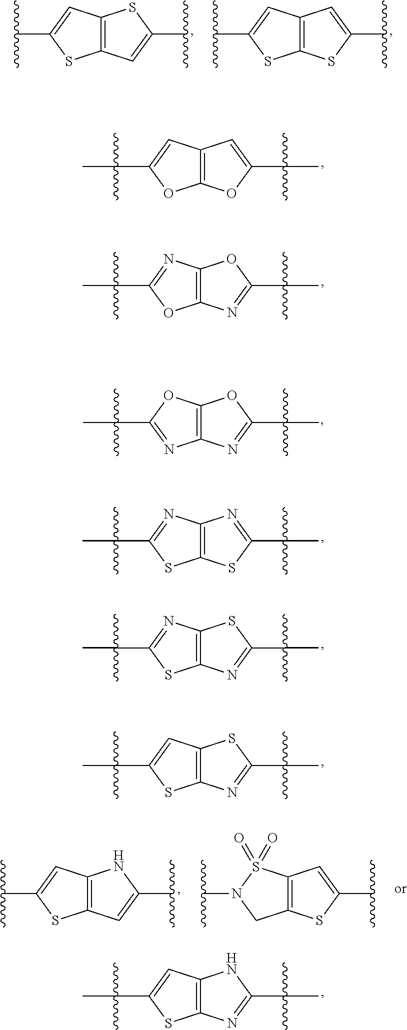

and wherein the rings in A are unsubstituted or substituted with one or more substituents selected from alkyl, haloalkyl, halogen, hydroxy, alkoxy, haloalkoxy, cyano, sulfonyl or oxo. In some such embodiments, A is selected from

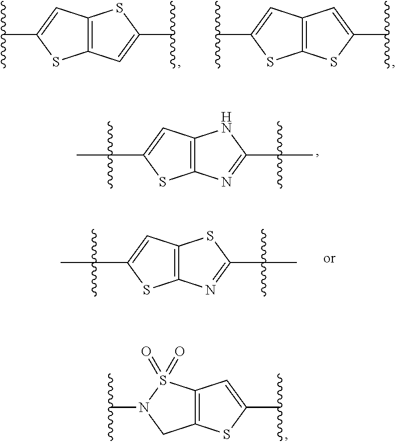

each optionally substituted with one or more substituents selected from the group consisting of alkyl, alkoxy, halogen, haloalkyl and cyano.

In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In still some other embodiments, m is 2.

In some embodiments, R² and R³ is hydrogen. In some other embodiments, one of R² and R³ is hydrogen and the other R² and R³ is aryl. In still some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond.

In some embodiments, R⁶ is R⁶ is hydrogen. In some other embodiments, R is C₁₋₃ alkyl.

In some embodiments, R¹⁰ is selected from C₁₋₃ alkyl or C₃₋₆ cycloalkyl.

In some embodiments, R⁴ is hydrogen. In some other embodiments, R⁴ is alkyl. In some further such embodiments, R⁴ is alkyl substituted with halogen.

In some embodiments,

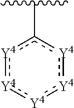

is also represented by

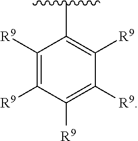

In some further embodiments, each of R⁹ is hydrogen. In some other embodiments, at least one R⁹ is selected from C₁₋₃ alkyl or halogen.

In some embodiments,

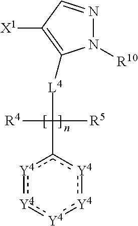

is selected from

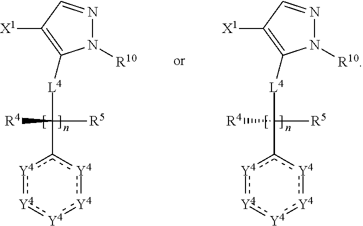

In some embodiments,

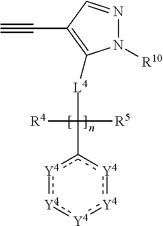

is selected from

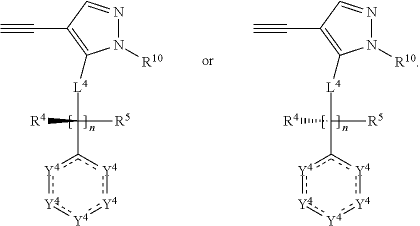

Some embodiments disclosed herein relate to compounds of formula (VII-1), wherein the structure of formula (VII-1) and the variables thereof including ring A, D, E, R¹, R², R^{2b}, R^{2c}, R³, R^{3b}, R^{3c}, R⁶, R^{6a}, R^{6b}, R^{6c}, R⁷, R⁸, R¹³, R¹⁴, R¹⁵, R¹⁶, R¹⁷, L⁵, Y², Y³, m, p, s and u are defined above in formula (VIIb).

In some embodiments, A is unsubstituted or substituted with one or more substituents selected from alkyl, haloalkyl, halogen, hydroxy, alkoxy, haloalkoxy, cyano, or oxo; D is selected from —OH,

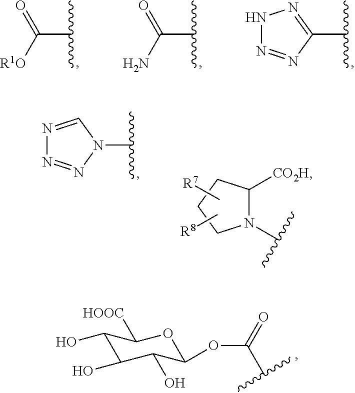

### —NR¹³SOₚR¹⁴, —C(O)—NR¹³SOₚR¹⁴,

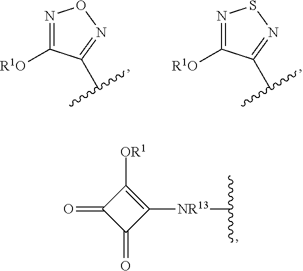

or carboxylic acid isosteres; E is absent; L⁵ is selected from a single bond, a —CH₂O— linker, a —CH═CH— linker, a —C≡C— linker,

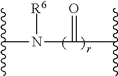

or a 4-7 membered heterocyclyl; R² and R³ are each independently selected from hydrogen, alkyl, aryl, or heteroaryl; or R² and R³ are joined together with the atom to which they are attached to form an optionally substituted cycloalkyl or an optionally substituted heterocyclyl; or R² is selected from hydrogen, alkyl, aryl, or heteroaryl and R³ is joined to an atom alpha to a point of attachment of L⁵ to A to form an optionally substituted cycloalkyl or an optionally substituted heterocyclyl; or R³ is selected from hydrogen, alkyl, aryl or heteroaryl and R² is joined to an atom alpha to a point of attachment of L⁵ to A to form an optionally substituted cycloalkyl or an optionally substituted heterocyclyl; and r is an integer of 0 or 1.

In some embodiments, the compound of formula (VII-1) is also represented by formula (VII-1A):

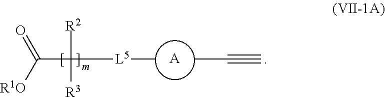

In some embodiments, ring A is selected from

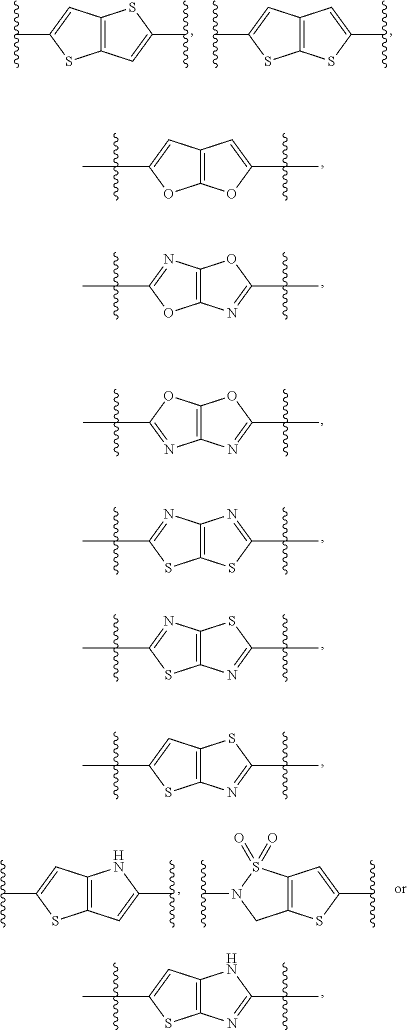

and wherein the rings in A are unsubstituted or substituted with one or more substituents selected from alkyl, haloalkyl, halogen, hydroxy, alkoxy, haloalkoxy, cyano, sulfonyl or oxo. In some further embodiments, ring A is selected from the group consisting of

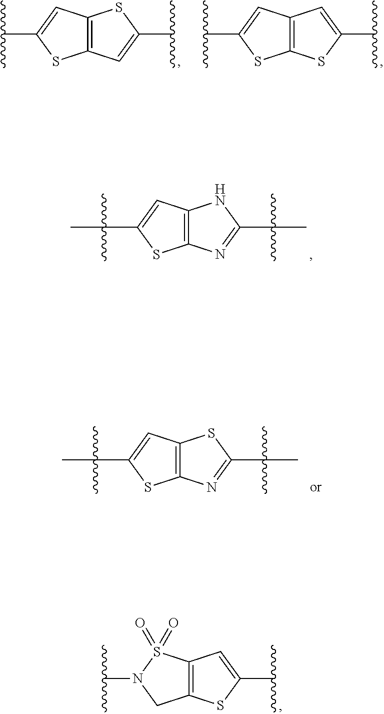

each optionally substituted with one or more substituents selected from the group consisting of alkyl, alkoxy, halogen, haloalkyl and cyano.

In some embodiments, R¹ is hydrogen or unsubstituted alkyl. In some other embodiments, R¹ is alkyl substituted with one or more substituents selected from the group consisting of alkoxy, C-amido, O-carboxy, and 6 membered heterocyclyl. In still some other embodiments, R¹ is optionally substituted aryl.

In some embodiments, m is 0. In some other embodiments, m is 1. In still some other embodiments, m is 2.

In some embodiments, R² and R³ is hydrogen. In some other embodiments, one of R² and R³ is hydrogen and the other R² and R³ is aryl. In still some other embodiments, R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl. In one embodiment, R² and R³ are joined together with the atom to which they are attached to form an optionally substituted cyclopropyl.

In some embodiments, L⁵ is a single bond.

In some embodiments, the acetylene group of the compound of formula (VII-1) or (VII-1A) is first activated by reacting with a tin reagent. In one embodiment, the tin reagent is n-Bu₃SnCl.

Some embodiments disclosed herein relate to compounds of formula (VII-2), wherein the structure of formula (VII-2) and the variables thereof including R⁴, R⁵, R⁶, R⁹, R¹⁰, L⁴, Y¹, Y⁴, W, X, n, p and q are defined above in formula (VIIb); and wherein R² and R³ are each independently selected from hydrogen, alkyl, aryl, or heteroaryl; or R² and R³ are joined together with the atom to which they are attached to form an optionally substituted cycloalkyl or an optionally substituted heterocyclyl.

In some embodiments, the compound of formula (VII-2) is also represented by formula (VII-2A):

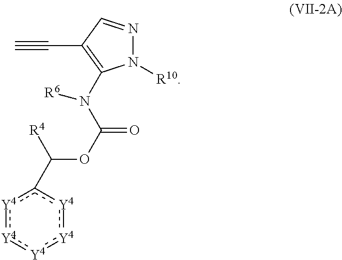

In some embodiments, R⁶ is R⁶ is hydrogen. In some other embodiments, R is C₁₋₃ alkyl.

In some embodiments, R¹⁰ is selected from C₁₋₃ alkyl or C₃₋₆ cycloalkyl.

In some embodiments, R⁴ is hydrogen. In some other embodiments, R⁴ is alkyl.

In some further such embodiments, R⁴ is alkyl substituted with halogen.

In some embodiments,

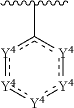

is also represented by

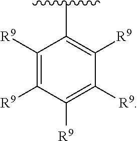

In some further embodiments, each of R⁹ is hydrogen. In some other embodiments, at least one R⁹ is selected from C₁₋₃ alkyl or halogen.

In some embodiments,

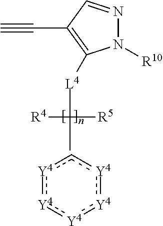

is selected from

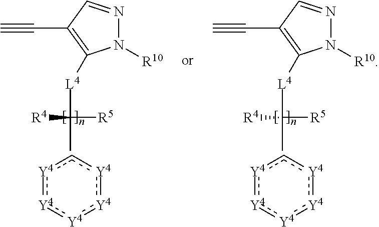

### EXAMPLES

Additional embodiments are disclosed in further detail in the following examples, which are not in any way intended to limit the scope of the claims.

### Example 1-A

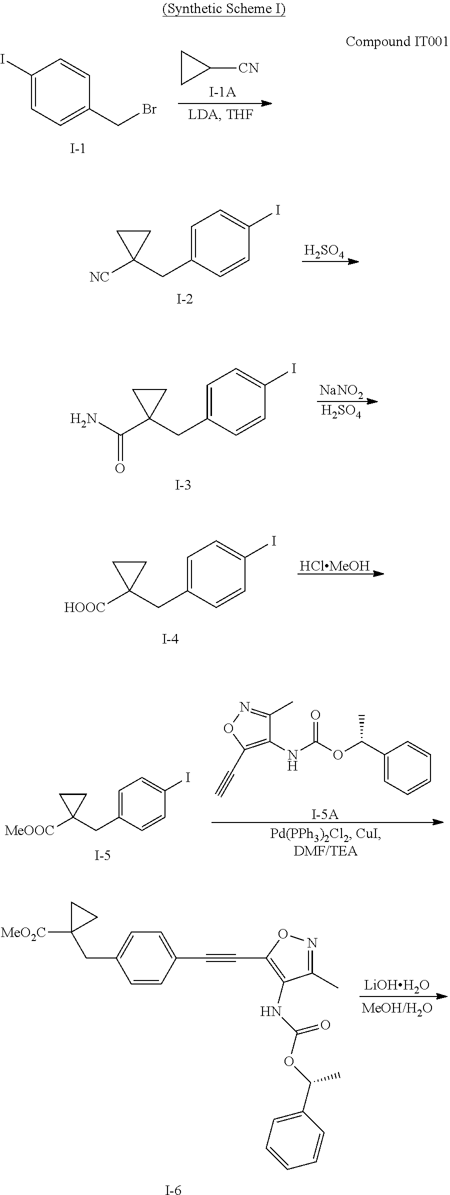

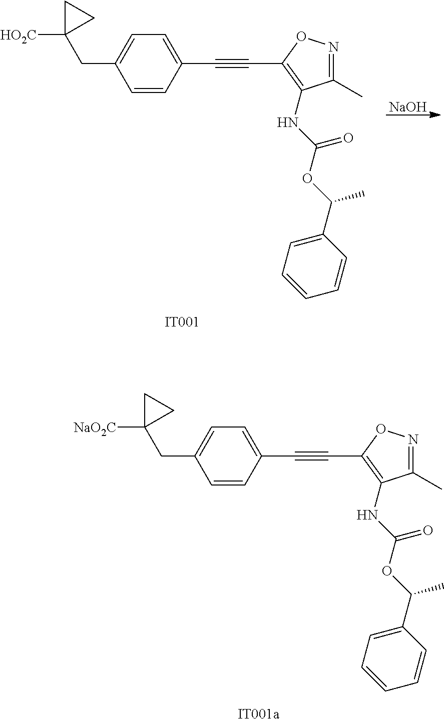

n-Butyl lithium (1.35 mL, 2.5M, 3.38 mmol) was added to a solution of diisopropylamine (0.45 mL, 3.24 mmol) in THF at −78° C. After 35 min, a solution of I-1A (189 mg, 2.82 mmol) in THF was added, and the bright yellow solution was stirred for 20 min. Then a solution of I-1 (1.0 g, 3.38 mmol) in THF (5 mL) was then added dropwise, and the reaction mixture was stirred with warming to rt over 1 h. The mixture was partitioned between water and EtOAc, dried over Na₂SO₄, and purified by column on chromatograph (PE:EA=100:3) to afford 1-2 (250 mg, yield 31.3%).

The solution of I-2 (250 mg, 0.88 mmol) in con.H₂SO₄ (12 mL) was heated to 100° C. for 2 hrs. The mixture was poured into ice-water and extracted with EtOAc. The organic layer was washed with H₂O, dried and concentrated to give I-3 (250 mg, crude yield 100%), which was used to next step directly.

To a stirred solution of I-3 (250 mg, 0.83 mmol) in con.H₂SO₄ (12 mL) was added in portions NaNO₂ (573 mg, 8.3 mmol) at 0° C. After addition, the mixture was heated to 100° C. for 2 hrs. The mixture was poured into ice-water and extracted with EtOAc. The organic layer was washed with H₂O, dried and concentrated to give I-4 (250 mg, crude yield 100%). which was used to next step directly.

The solution of I-4 (250 mg, 0.83 mmol) in MeOH/HCl (10 mL) was heated to 60° C. overnight. After concentrated, the residue was extracted with EtOAc, washed with aq. NaHCO₃ and brine. The mixture was poured into ice-water to afford a white precipitate. The organic layer was dried and concentrated to give I-5 (120 mg, yield 45.8%). MS (ESI) m/z (M+H)⁺ 316.9.

To a stirred mixture of I-5 (50 mg, 0.165 mmol), I-5A (44.4 mg, 0.165 mmol) and CuI (1.6 mg, 0.008 mmol) in DMF (3 mL) and TEA (1 mL) was added Pd(PPh₃)₂Cl₂ (12 mg, 0.02 mmol). The reaction mixture was flushed with Ar and stirred at rt for overnight. The mixture was diluted with EtOAc (20 mL), washed with water and brine. The organic layer was dried over Na₂SO₄, filtered and concentrated. The residue was purified by prep-TLC (PE:EA=5:1) to give I-6 (20 mg, yield 28.7%). MS (ESI) m/z (M+Na)⁺481.1.

To a solution of I-6 (46 mg, 0.10 mmol) in MeOH (6.0 mL) was added water (2.0 mL) and lithium monohydrate (21.2 mg, 0.50 mmol). The reaction mixture was stirred at rt overnight. The mixture was adjust to pH=4.0 with 1N hydrochloride solution, and extracted with EtOAc. The combined organic phase was dried over MgSO₄ and concentrated. The residue was purified by prep-HPLC to give IT001 (45 mg, yield 100%). MS (ESI) m/z (M+H)⁺ 445.1.

To a solution of I-7 (46 mg, 0.104 mmol) in MeOH (2 mL) was added 0.05N NaOH solution (2.08 mL). The reaction mixture was stirred for 30 minutes. The mixture was lyophilized to give IT001a. ¹H NMR (400 MHz, Methanol-d₄): δ 7.31-7.41 (m, 9H), 5.84 (q, 1H), 3.02 (s, 2H), 2.20 (s, 3H), 1.60 (d, J=6.4 Hz, 3H), 1.15-1.17 (m, 2H), 0.61-0.63 (m, 2H). MS (ESI) m/z (M+H)⁺ 445.1.

### Example 1-B

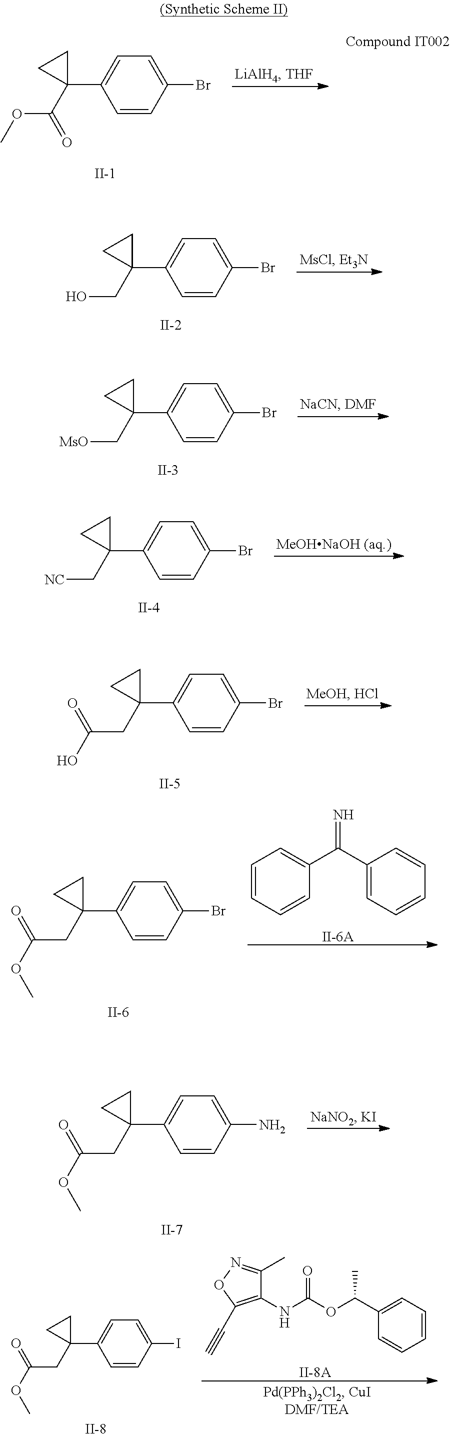

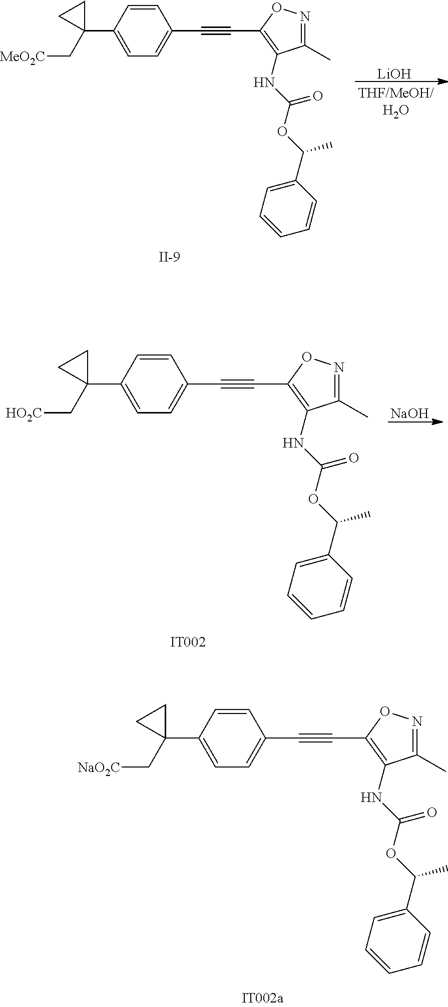

To a solution of II-1 (5 g, 19.7 mmol) in THF (150 mL) at −4° C. was LiAlH₄ (1.54 g, 39.4 mmol) portionwise over 30 min. The reaction was stirred for 30 min, and then water (20 mL) was added, followed by 4N NaOH (15 mL) and additional water (50 mL). The mixture was stirred for 15 minutes and filtered. The filtrate was extract with EtOAc, the combined organic layers were dried over Na₂SO₄, concentrated in vacuo. The residue was purified by column chromatography (PE:EA=5:1) to afford II-2 (3.1 g, yield 70%).

II-2 (5 g 22 mmol) in DCM (80 mL) at −78° C. was treated with Et₃N (4.45 g, 44 mmol), followed by MsCl (2.5 g, 22 mmol). The reaction was stirred for 1 hour at −78° C., and then warmed to 0° C. and stirred for 2 h. The mixture was diluted with 1 N aqueous HCl and extracted with DCM. The combined organic extracts were dried over anhydrous MgSO₄, filtered and concentrated in vacuo. II-3 was used directly without further purification.

The mixture of II-3 (9 g, 29.6 mmol) in DMF (80 mL) was added NaCN (2.78 g, 59.2 mmol), and the reaction mixture was stirred at 70° C. for 3 h. The mixture was diluted with EtOAc and water, and the organic layer was separated, dried and concentrated. The residue was purified by chromatography on silica gel (PE:EA=5:1) to afford II-4 (5.35 g, yield 77%).

The mixture of II-4 (6 g, 22.3 mmol) and NaOH (10 g, 0.25 mol) was dissolved in MeOH (50 mL) and H₂O (50 mL) then the reaction was heated to 60° C. for 16 h. After concentrated, the aqueous layer was adjust to pH=3 with 1N HCl, and extracted with EtOAc, the organic layer was separated, dried and concentrated to afford II-5, which was used in the next step without further purification.

The mixture of II-5 (4 g, 15.7 mmol) in HCl/MeOH (4N, 30 mL) was stirred at reflux for 18 hours. After evaporated of the solvent, the residue was diluted with water and extracted with DCM. The combined organic extracts were dried over anhydrous MgSO₄, filtered and concentrated in vacuo. The residue was purified by column chromatography (PE:EA=10:1) to afford II-6 (2.4 g, yield: 56.8%).

A mixture of II-6 (1 g, 3.72 mmol), II-6A (811 mg, 4.46 mmol), Pd(OAc)₂ (83 mg, 0.37 mmol), BINAP (18 mg, 0.03 mmol) and Cs₂CO₃ (2.4 g, 7.44 mmol) in toluene (120 mL) was vigorously stirred under nitrogen atmosphere at 110° C. for 18 h. After removal of the solvent, the residue was diluted with water and extracted with EtOAc. The combined organic layers were dried over MgSO₄ and evaporated. The residue was purified by column chromatography (PE:EA=10:1) to afford II-7 (0.75 g, yield 55%).

To a solution of p-TsOH (753 mg, 4.38 mmol) in CH₃CN (80 mL) was added II-7 (300 mg, 1.46 mmol). The reaction mixture was cooled to 5° C. and a solution of NaNO₂ (202 mg, 2.93 mmol) and KI (606 mg, 3.65 mmol) in H₂O (9 mL) was added dropwise. The mixture was stirred for 2 h at rt. After removal of the solvent, the residue was diluted with water and extracted with EtOAc. The combined organic layers were dried over MgSO₄ and evaporated. The residue was purified by column chromatography (PE:EA=10:1) to afford II-8 (0.116 g, yield: 25%). MS (ESI) m/z (M+H)⁺ 317.0.

II-9, IT002, and IT002a were prepared following the similar procedure described in the preparation of 1-6, IT001 and IT001a. IT002: MS (ESI) m/z (M+H)⁺ 445.2. IT002a: ¹H NMR (DMSO-d₆ 300 MHz) δ 7.32-7.38 (m, 9H), 5.76-5.80 (m, 1H), 2.28 (s, 2H), 2.15 (s, 3H), 1.52 (d, J=6.0 Hz, 3H), 0.98 (br, 2H), 0.81 (br, 2H). MS (ESI) m/z (M+H)⁺ 445.2.

IT003 and IT003a were prepared following the similar synthetic scheme of IT002, using methyl 1-(6-bromonaphthalen-2-yl)cyclopropanecarboxylate in place of II-1. IT003: MS (ESI) m/z (M+H)⁺ 495.2. IT003a: ¹H NMR (DMSO-d₆, 400 MHz) δ 8.09 (s, 1H), 7.79-7.86 (m, 3H), 7.35-7.53 (m, 7H), 5.80-5.82 (q, 1H), 2.44 (s, 2H), 1.54 (d, J=6.4 Hz, 3H), 1.01 (br, 2H), 0.90 (br, 2H). MS (ESI) m/z (M+H)⁺ 495.2.

IT065 was prepared following the similar synthetic scheme of IT002, using 1-((6-bromonaphthalen-2-yl)methyl)cyclopropanecarbonitrile in place of II-4, which was obtained in two steps from bromination of (6-bromonaphthalen-2-yl)methanol to form 2-bromo-6-(bromomethyl)naphthalene, followed by reacting with cyclopropanecarbonitrile. IT065: MS (ESI) m/z (M+H)⁺ 495.1. Sodium salt IT065a: ¹H NMR (400 MHz, Methanol-d₄) δ 8.03 (s, 1H), 7.76-7.83 (m, 3H), 7.57 (d, J=8.0 Hz, 1H), 7.27-7.45 (m, 6H), 5.84-5.87 (q, 1H), 3.18 (s, 2H), 2.22 (s, 3H), 1.61 (br, 3H), 1.21 (br, 2H), 0.70 (br, 2H). MS (ESI) m/z (M+H)⁺ 495.1.

### Example 1-C

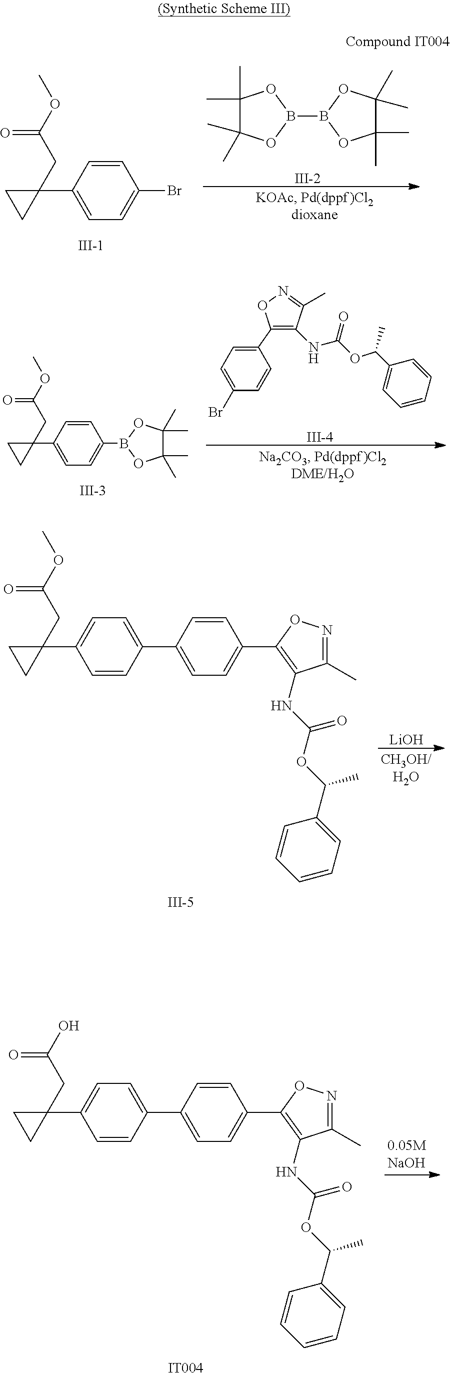

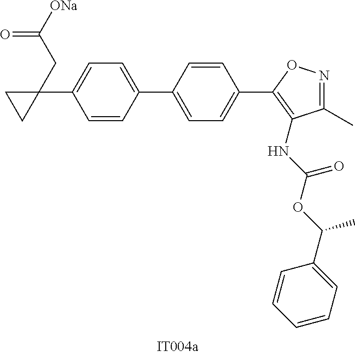

To a stirred solution of III-1 (240 mg, 0.94 mmol), III-2 (286.9 mg, 1.13 mmol), KOAc (184.5 mg, 1.88 mmol) in dioxane (15 mL) was added Pd(dppf)Cl₂ (103.3 mg, 0.14 mmol) The mixture was purged with nitrogen for 5 min and heated to reflux for overnight. After being cooled to rt, the mixture was diluted with water (8 mL) and extracted with EtOAc. The combined organic layers were washed with brine, and concentrated under vacuo. The residue was purified by column chromatography on silica gel (PE:EA=10:1) to give III-3 (190.9 mg, yield 64.2%).

To a stirred solution of III-3 (190.9 mg, 0.64 mmol), III-4 (290.7 mg, 0.72 mmol), Na₂CO₃ (128.1 mg, 1.21 mmol) in DME/H₂O (20 mL, v/v=3:1) was added Pd(dppf)Cl₂ (66.4 mg, 0.09 mmol) under nitrogen. Then the solution was heated to reflux for 4 hours. After concentrated, H₂O (5 mL) was added, and the mixture was extracted with EtOAc. The organic layer was combined and washed with brine, dried over Na₂SO₄, concentrated in vacuo. The residue was purified by column chromatography on silica gel (PE:EA=1:1) to afford III-5 (256 mg, yield: 26.9%).

IT004 and IT004a were prepared following the similar procedure described in the synthesis of IT001 and IT001a. IT004a: ¹H NMR (DMSO-d₆, 400 MHz): δ 7.74-7.77 (m, 4H), 7.54-7.56 (m, 2H), 7.36-7.42 (m, 7H), 5.74-5.75 (q, 1H), 2.32 (br, 2H), 2.12 (s, 3H), 1.54 (br, 2H), 0.96 (br, 2H), 0.79 (br, 2H). MS (ESI) m/z (M+H)⁺ 497.2.

### Example 2-A

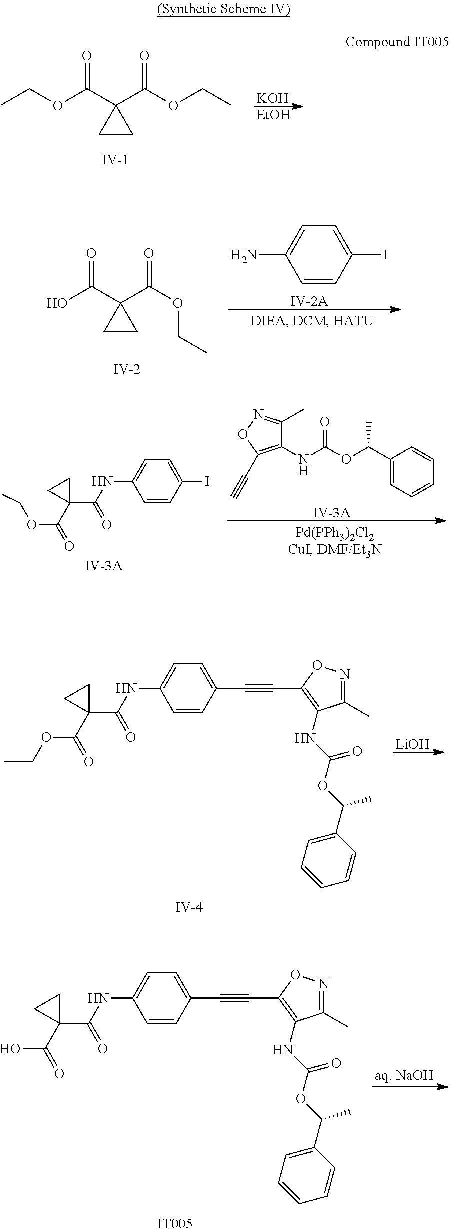

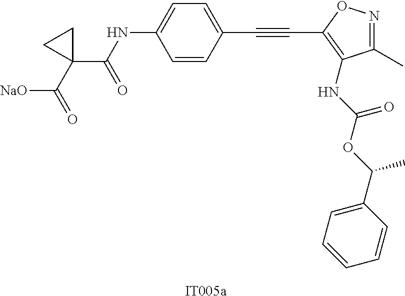

KOH (2.8 g, 50 mmol), was added to a solution of IV-1 (9.3 g, 50 mmmol) in 200 mL EtOH. The reaction mixture was stirred at rt overnight. After concentrated under reduced pressure, the residue was re-dissolved in 50 mL of NaHCO₃ solution (w/w=5%) and extracted with DCM. The aqueous layer was separated, and adjusted pH to 2 with 1N HCl, and extracted with EtOAc. The combined organic layer was dried and concentrated to afford IV-2 (6.0 g, yield 79%), which was used to next step directly.

IV-2A (2.19 g, 10 mmmol) was added to a mixture of IV-2 (1.58 g, 10 mmmol) and HATU (4.56 g, 12 mmmol) in 20 mL of DCM. The reaction mixture was stirred at rt overnight. Then water (15 mL) was added and extracted with DCM. The organic layer was separated, dried and concentrated. The residue was purified by column (PE/EA=10/1) to afford IV-3 (1.2 g, yield 33.4%).

IV-4, IT005, and IT005a were prepared following the similar procedure described in the preparation of 1-6, IT001 and IT001a. IT005: MS (ESI) m/z (M+H)⁺ 474.1. IT005a: ¹H NMR (DMSO-d₆, 400 MHz): δ 7.23 (d, J=8.4 Hz, 2H), 7.28-7.41 (m, 7H), 5.81-5.83 (q, 1H), 2.18 (m, 3H), 1.57 (d, J=6.4 Hz, 3H), 1.47-1.49 (m, 2H), 1.40-1.43 (m, 2H). MS (ESI) m/z (M+H)⁺ 474.1.

### Example 2-B

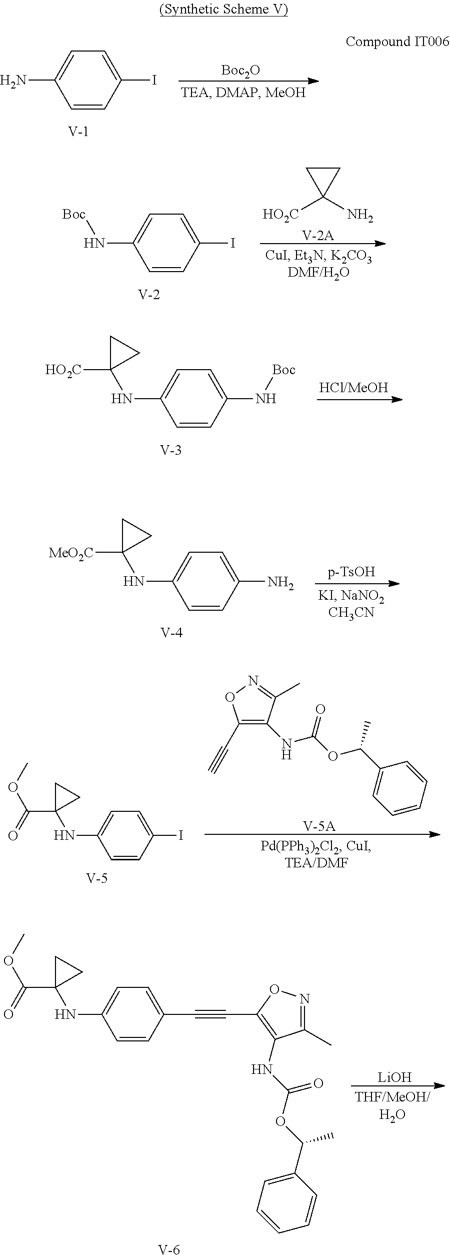

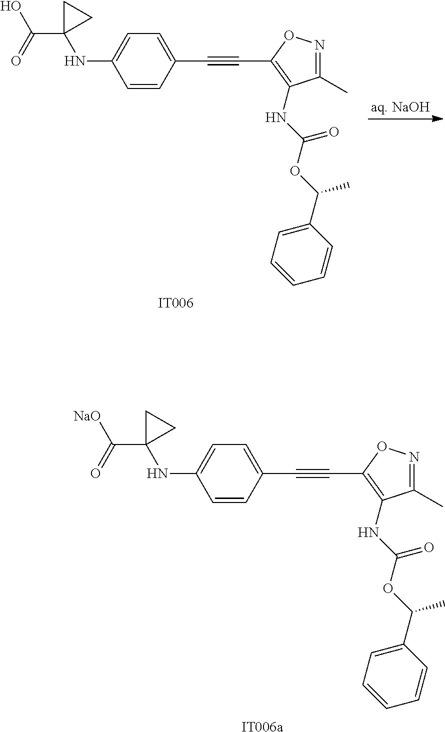

To a solution of V-1 (10 g, 45.66 mmol), TEA (9.22 g, 91.23 mmol) and DMAP (50 mg) in MeOH (100 mL) was added di-tert-butyl dicarbonate (19.8 g, 50.2 mmol). The mixture was heated to 50° C. overnight. After completion of the reaction, the mixture was concentrated, the residue was purified by column chromatography (PE/EA=10/1) to afford V-2 (8.47 g, yield 58.2%).

To a solution of V-2 (4 g, 12.53 mmol) and V-2A (1.9 g, 18.80 mmol) in DMF/H₂O (40 mL, v/v=3/1) was added K₂CO₃ (5.2 g, 37.6 mmol), Et₃N (0.18 mL, 1.25 mmol) and CuI (0.48 g, 2.51 mmol). The reaction mixture was heated to 110° C. and stirred overnight. After completion of the reaction, the mixture was diluted with H₂O, extracted with EtOAc, the combined organic layer was washed with brine, dried and concentrated to afford V-3 (3.9 g, crude), which was used to next step directly.

A mixture of crude V-3 (3.9 g) in 4 N HCl in methanol (60 mL) was heated to reflux for 4 hours. The mixture was concentrated. The residue was dissolved in ethyl acetate, washed with saturated NaHCO₃, dried and concentrated. The residue was purified by flash column chromatography on silica gel (PE:EA=2/1) to afford V-4 (0.8 g, yield 31% over two steps).

To a stirred solution of p-TsOH.H₂O (2.2 g, 11.64 mmoL) in CH₃CN (15 mL) was added V-4 (800 mg, 3.88 mmol. The resulting suspension of amine salt was cooled to 5° C. and a solution of NaNO₂ (535 mg, 7.76 mmol) and KI (1.61 mg, 9.70 mmol) in H₂O was added dropwise. The mixture was stirred overnight at rt. The mixture was concentrated in vacuum. The residue was partitioned between ethyl acetate and saturated NaHSO₃. The organic layer was washed with brine, dried and concentrated. The residue was purified by flash column chromatography on silica gel (PE:EA=8/1) to afford V-5 (300 mg, yield 25%).

V-6, IT006, and IT006a were prepared following the similar procedure described in the preparation of 1-6, IT001 and IT001a. IT006a: ¹H NMR (400 MHz, DMSO-d₆): δ 9.30 (br, 1H), 7.31-7.38 (m, 5H), 7.14 (d, J=8.8 Hz, 2H), 6.82 (s, 1H), 6.58 (d, J=8.8 Hz, 2H), 5.77 (q, J=6.4 Hz, 1H), 2.12 (s, 3H), 1.52 (d, J=6.48 Hz, 3H), 1.21-1.24 (m, 2H), 0.58-0.59 (m, 2H). MS (ESI) m/z (M+H)⁺ 446.1.

### Example 3-A

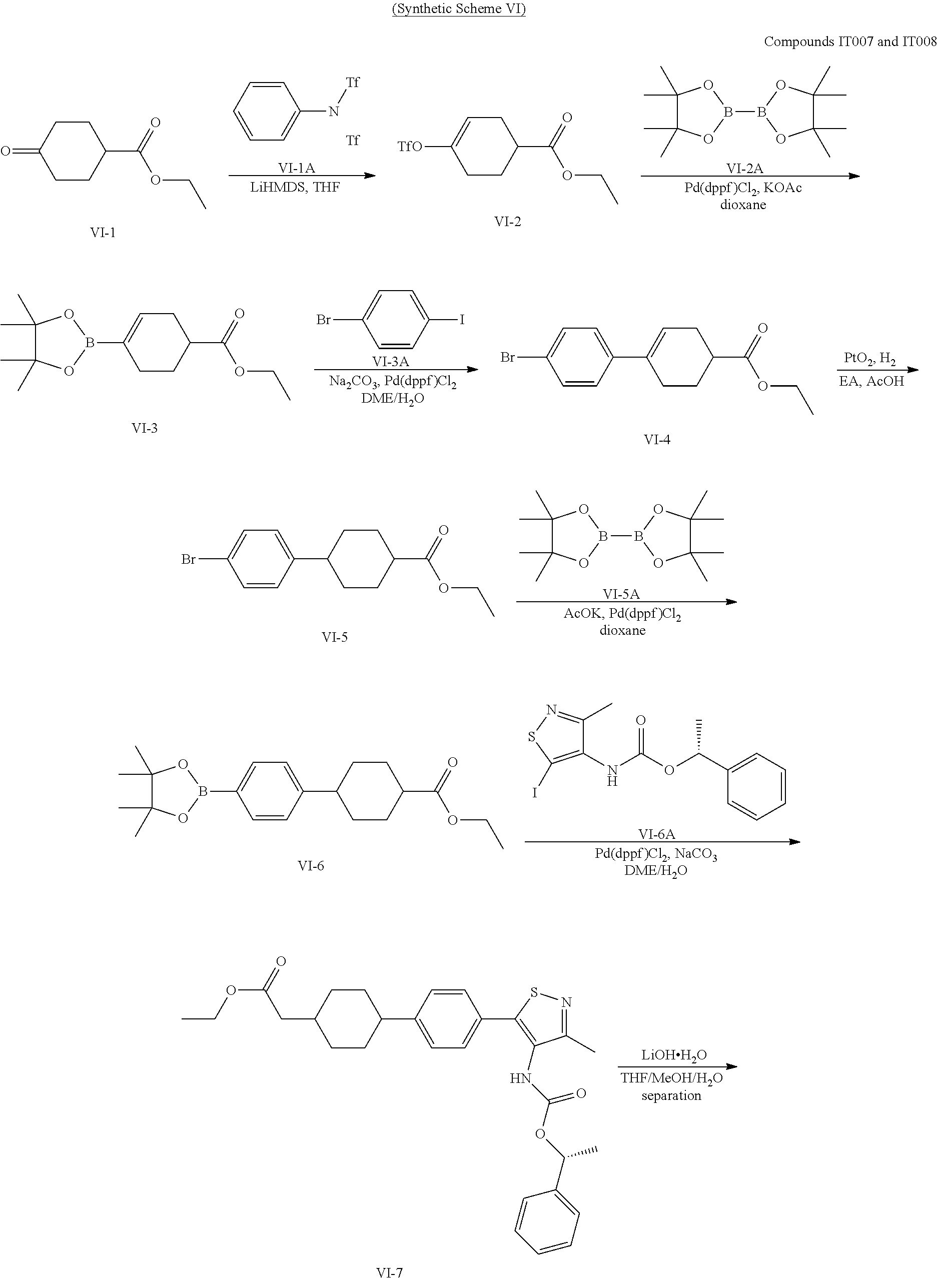

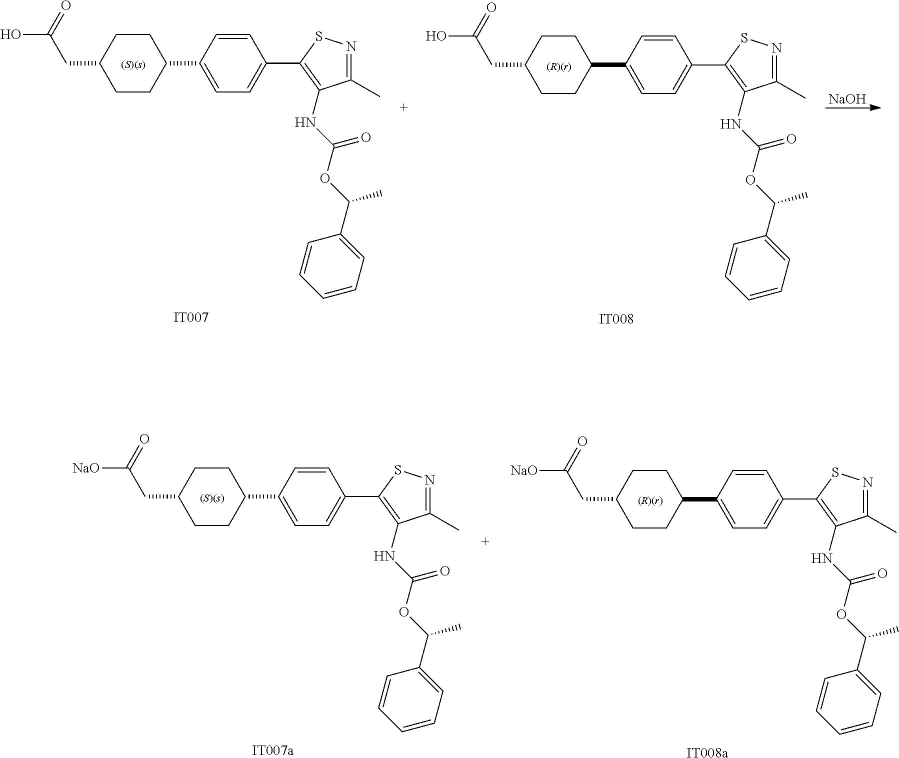

To a stirred solution LiHMDS (23.5 g, 141 mmol) in THF (400 mL) was added VI-1 (20 g, 128.2 mmol) at −78° C. After 30 min VI-1A (50 g, 141 mmol) was added to the dark brown solution. After stirred for 30 min at −78° C., the mixture was allowed to warm to rt. The mixture was diluted with EA (500 mL×3) washed with aq NaHCO₃ (300 mL), and the combined organic layer was washed with brine, dried over Na₂SO₄, and concentrated. The residue was purified by column chromatography on silica gel (PE:EA=5:1) to afford VI-2 (20 g, yield: 54.2%).

The mixture of VI-2 (3 g, 10.4 mmol), VI-2A (3.17 g 12.5 mmol), KOAc (2.0 g, 20.8 mmol) and Pd(dppf)Cl₂ (0.3 g) in dioxane (60 mL) was heated to reflux under nitrogen overnight. After concentrated under reduced pressure, the residue was partitioned between H₂O (60 mL) and DCM (60 mL), the aqueous phase was extracted with DCM, and the combined organic layer was washed with brine, dried over Na₂SO₄, and concentrated. The residue was purified by column chromatography on silica gel (PE:EA=5:1) to afford VI-3 (1.1 g, yield: 37.9%).

The mixture of VI-3A (3.0 g, 10.7 mmol), VI-3 (3 g, 10.7 mmol), Na₂CO₃ (2.7 g, 21.4 mmol) and Pd(dppf)Cl₂ in DME/H₂O (90 mL, v/v=3:1) was heated to reflux under nitrogen overnight. After concentrated under reduced pressure, the mixture was partitioned between H₂O (60 mL) and DCM (60 mL), the aqueous phase was extracted with DCM, and the combined organic layer was washed with brine, dried over Na₂SO₄, concentrated. The residue was purified by column chromatography on silica gel (PE:EA=10:1) to afford VI-4 (1.5 g, yield: 45.45%).

The mixture of VI-4 (1.8 g, 5.8 mmol), AcOH (40 mg, 0.58 mmol) and PtO₂ (180 mg) in EtOAc (20 mL) was stirred at rt under H₂ (45 psi) overnight. After concentrated, the residue was partitioned between H₂O (30 mL) and DCM (30 mL), the aqueous phase was extracted with DCM, and the combined organic layer was washed with aq. NaHCO₃, brine, dried over Na₂SO₄, and concentrated. The residue was purified by column chromatography on silica gel to (PE:EA=7:1) afford VI-5 (1.1 g, yield: 60.77%).

VI-6 was prepared following the similar procedure for the synthesis of VI-3.

VI-7, IT007, IT008, IT007a and IT008a were prepared following the similar procedure described in the preparation of 1-6, IT001 and IT001a. IT007 and IT008 were obtained by chiral separation: MS (ESI) m/z (M+H)⁺ 465.2. IT007a: ¹H NMR (Methanol-d₄, 400 MHz): δ 7.27-7.40 (m, 9H), 5.69-5.79 (m, 1H), 2.54-2.61 (m, 1H), 2.52 (br, 1H), 2.27-2.31 (m, 5H), 1.71-1.85 (m, 2H), 1.57-1.69 (m, 7H). MS (ESI) m/z (M+H)⁺ 465.2. IT008a: ¹H NMR (Methanol-d₄, 400 MHz): δ 7.26-7.41 (m, 9H), 5.69-5.80 (m, 1H), 2.55-2.61 (m, 1H), 2.32 (s, 3H), 2.20-2.25 (m, 1H), 2.07-2.10 (m, 2H), 1.91-1.94 (m, 2H), 1.51-1.65 (m, 7H). MS (ESI) m/z (M+H)⁺ 465.2.

### Example 3-B

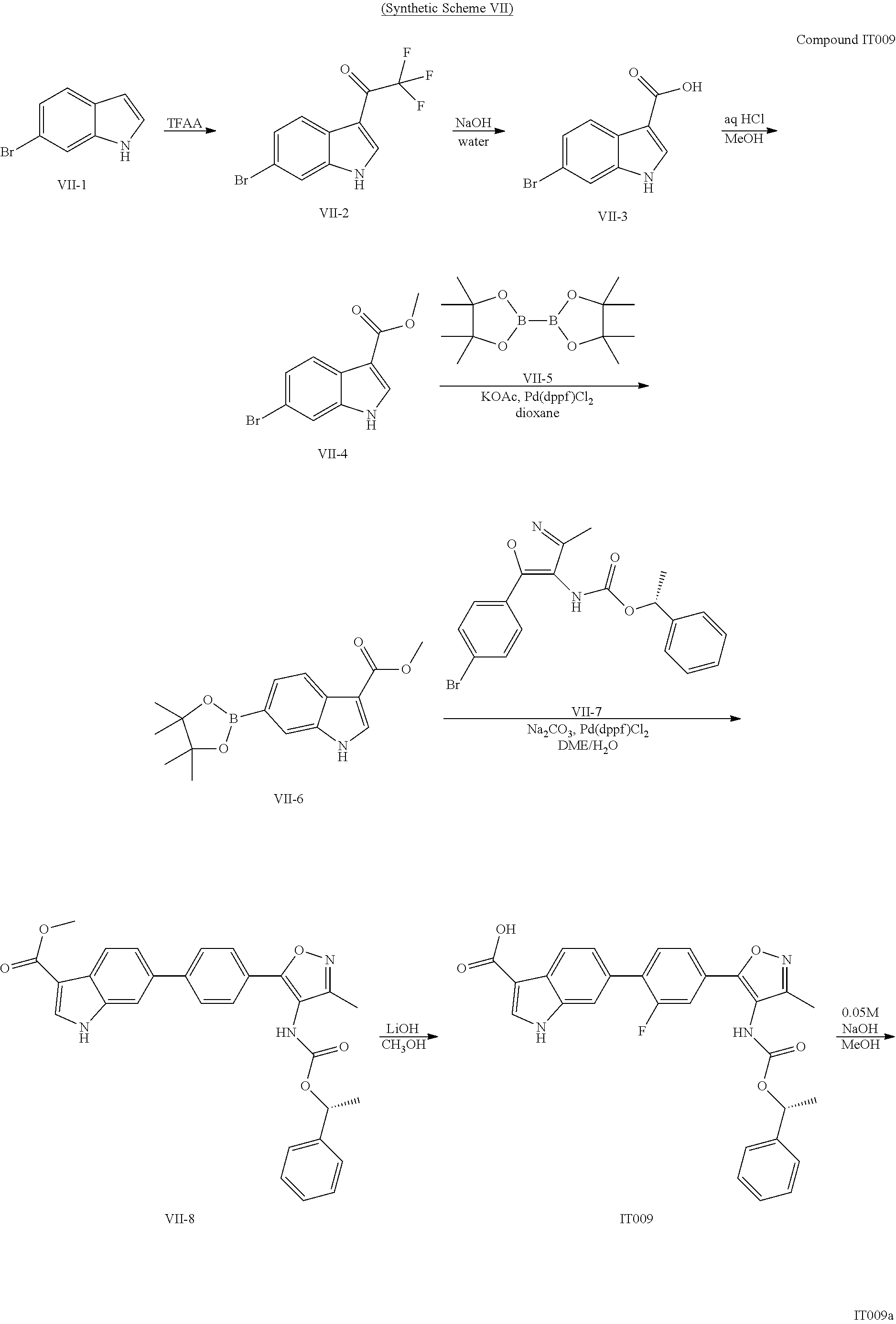

To the solution of VII-1 (3.9 g, 0.02 mol) in dry DMF (40 mL) was added TFAA (4.8 g, 0.028 mol) by dropwise at 0° C. and the reaction mixture was stirred for 3 hs at the same temperature. The solution was poured into water and the appeared solid was collected by filtration. The solid was washed with DCM to afford VII-2 (4.5 g, yield 77%) as a yellow solid.

To a stirred solution of VII-2 (1.7 g, 5.8 mmol) and NaOH (2.3 g, 58 mmol) in THF/water=1:1 (40 mL) was heated to reflux and stirred for 24 hours. The solvent was removed and the residue was added 2M HCl to adjust pH=2, the solid was collected and dried to give VII-3 (0.7 g, yield 50%) as a yellow solid.

To a stirred solution of VII-3 (0.86 g, 3.6 mmol) in MeOH (30 mL) was added aq. HCl (0.5 mL) under nitrogen. After the addition, the solution was heated to reflux under nitrogen for 2 hours. The solvent was removed by reduced pressure and the residue was added sat. NaHCO₃ to adjust to pH=9 and the solution was extracted with DCM, the combine organic layer was dried and concentrated in vacuum to afford VII-4 (0.71 g, 78%) as a yellow solid which was used for next step directly.

VII-6 and VII-8 were obtained following the similar procedure as described for the preparation of VI-6 and VI-7.

IT009 and IT009a were prepared following the similar procedure described in the preparation of IT001 and IT001a. IT009: MS (ESI) m/z (M+Na)⁺482.1. IT009a: ¹H NMR (Methanol-d₄, 400 MHz): δ 8.29 (d, J=8.4 Hz, 1H), 7.65-7.81 (m, 6H), 7.32-7.44 (m, 6H), 5.80-5.82 (q, 1H), 2.18 (s, 3H), 1.56 (d, J=6.4 Hz, 3H). MS (ESI) m/z (M+Na)⁺482.1.

### Example 3-C

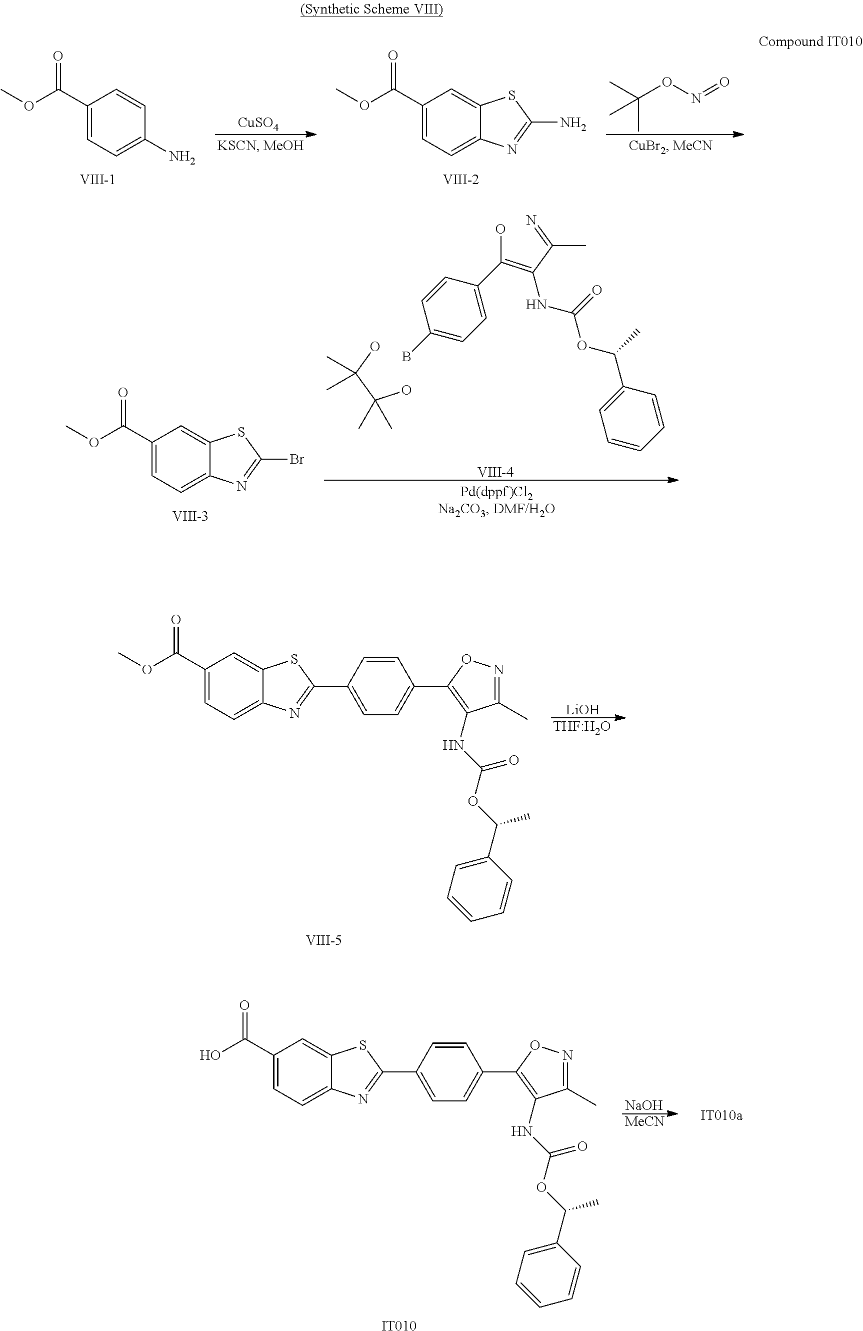

To a solution of VIII-1 (10 g, 66 mmol) in MeOH (100 mL) was added KSCN (51.2 g, 0.53 mol) and CuSO₄ (38.4 g, 0.24 mol). The reaction mixture was heated to 80° C. overnight. The mixture was diluted with EtOAc, washed with brine, dried over Na₂SO₄, filtered and concentrated. The residue was purified by column chromatography (PE:EA=2:1) to give VIII-2 (5 g, yield: 36%).

To a stirred mixture of VIII-2 (600 mg, 2.28 mmol) and CuBr₂ (775 mg, 3.46 mmol) in MeCN (9 mL) was added tert-butyl nitrite (445 mg, 4.32 mmol). The reaction mixture was stirred at rt overnight. The mixture was diluted with EtOAc (40 mL), washed with water and brine, dried over Na₂SO₄, filtered and concentrated. The residue was purified by column (PE:EA=5:1) to give VIII-3 (140 mg, yield: 18%).

To a stirred mixture of VIII-3 (200 mg, 0.738 mmol), VIII-4 (400 mg, 0.88 mmol), and Na₂CO₃ (233 mg, 2.198 mmol) in DME (6 mL) and H₂O (2 mL) was added Pd(dppf)Cl₂ (53.9 mg, 0.0738 mmol). The reaction mixture was flushed with nitrogen and heated to 80° C. overnight. The mixture was diluted with EtOAc (40 mL), washed with water and brine, dried over Na₂SO₄, filtered and concentrated. The residue was purified by column chromatography (PE:EA=10:1) to give VIII-5 (50 mg, yield: 13.15%). MS (ESI) m/z (M+H)⁺ 514.1.

IT010 and IT010a were prepared following the similar procedure described in the preparation of IT001 and IT001a. IT010: MS (ESI) m/z (M+H)⁺ 500.1. IT010a: ¹HNMR (DMSO-d₆, 400 MHz) δ 8.58 (s, 1H), 8.12-8.17 (m, 3H), 8.01 (d, J=8.8 Hz, 1H), 7.88 (d, J=7.6 Hz, 2H), 7.36-7.45 (m, 5H), 5.81-5.83 (m, 1H), 2.20 (s, 3H), 1.63 (d, J=6.0 Hz, 3H), MS (ESI) m/z (M+H)⁺ 500.1.

### Example 4-A

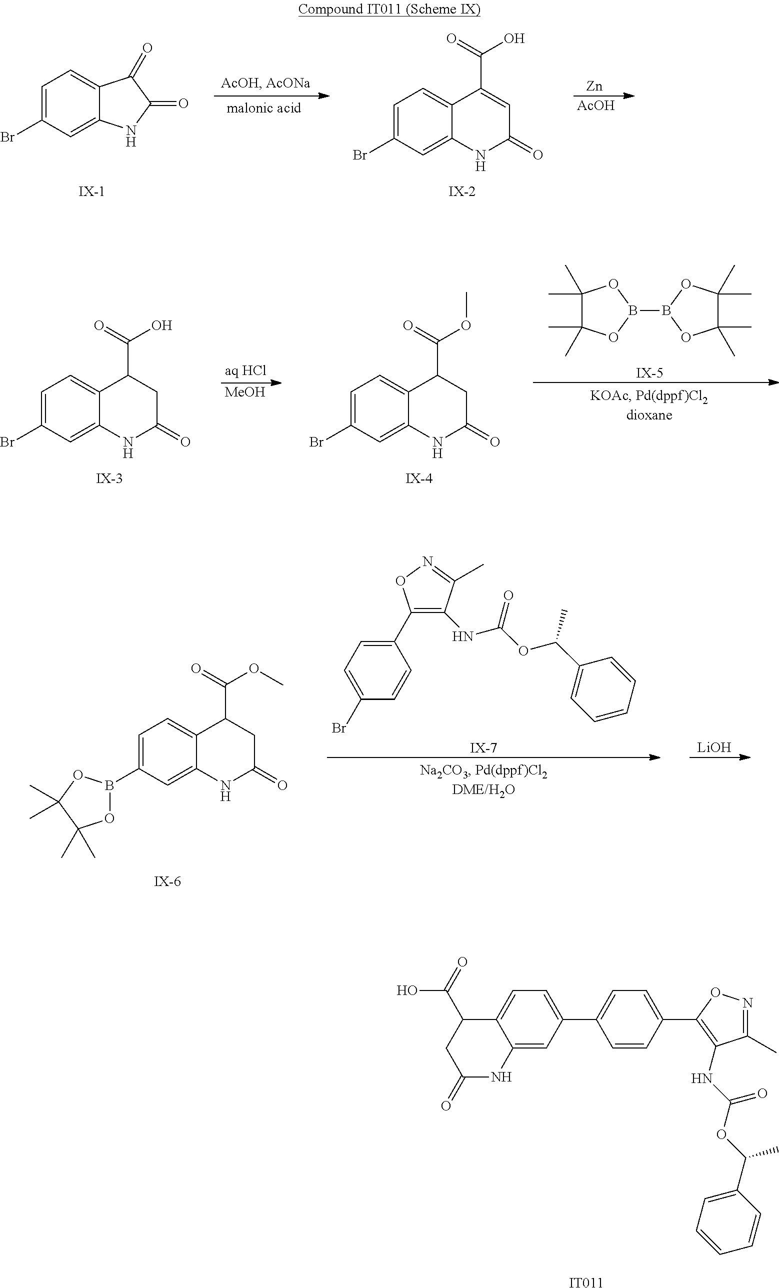

The mixture of IX-1 (6.5 g, 28.7 mmol), malonic acid (3.3 g, 31.7 mmol), NaOAc (2.95 g, 36 mmol) in AcOH (60 mL) were stirred at rt. After 6 hrs, NaOAc (2.95 g, 36 mmol) was added additional, then refluxed overnight. After cooling, the mixture was filtered and the filtrate was washed with water and EtOAc, then dried under reduced pressure to afford IX-2 (5 g, yield 66%) as a brown oil, which was used for next step directly.

The solution of IX-2 (3 g, 11 mmol) and Zn (6 g, 88 mmol) in AcOH (40 mL) was heated to reflux and stirred for 24 hrs. The reaction was filtered and the filtrate was concentrated, the residue was added Sat.NaHCO₃ to adjust pH=9 and extracted with DCM, the aqueous layer was added aq. HCl to adjust pH=5. The solid was collected to afford IX-3 (1 g, yield 33%) as a brown solid.

To a stirred solution of IX-3 (1.05 g, 3.7 mmol) in MeOH (30 mL) was added aq HCl (0.5 mL) under nitrogen. After the addition, the solution was heated to reflux under nitrogen for 2 hrs. The solvent was removed by reduced pressure. The residue was added Sat.NaHCO₃ (10 mL) to adjust pH=9, extracted with EtOAc, the combine organic layers was dried over NaSO₄, concentrated in vacuum to afford crude IX-4 (0.9 g, yield 81%) as a yellow solid, which was used for next step directly.

IX-6 was prepared following the similar procedure described in the preparation of VI-3 as a brown solid. IT011 was prepared following the similar procedure described in the preparation of VIII-5. ¹H NMR (Methanol-d₄, 400 MHz): δ 7.82 (d, J=8.0 Hz 2H), 7.68 (d, J=7.6 Hz, 2H), 7.13-7.47 (m, 8H), 5.85 (m, 1H), 4.04 (t, 1H), 2.92 (m, 1H), 2.80-2.89 (m, 2H), 2.20 (s, 3H), 1.63 (d, J=5.6 Hz, 3H). The sodium salt IT011a was prepared following the similar prodecure described in the preparation of IT001a. ¹H NMR (Methanol-d₄, 400 MHz): δ 7.78 (d, J=7.6 Hz, 2H), 7.64 (d, J=7.8 Hz, 2H), 7.13-7.47 (m, 8H), 5.81 (m, 1H), 3.79 (t, J=6.4 Hz, 1H), 2.92 (m, 1H), 2.65-2.73 (m, 1H), 2.18 (s, 3H), 1.61 (d, J=5.6 Hz, 3H). MS (ESI) m/z (M+Na)⁺512.1.

### Example 4-B

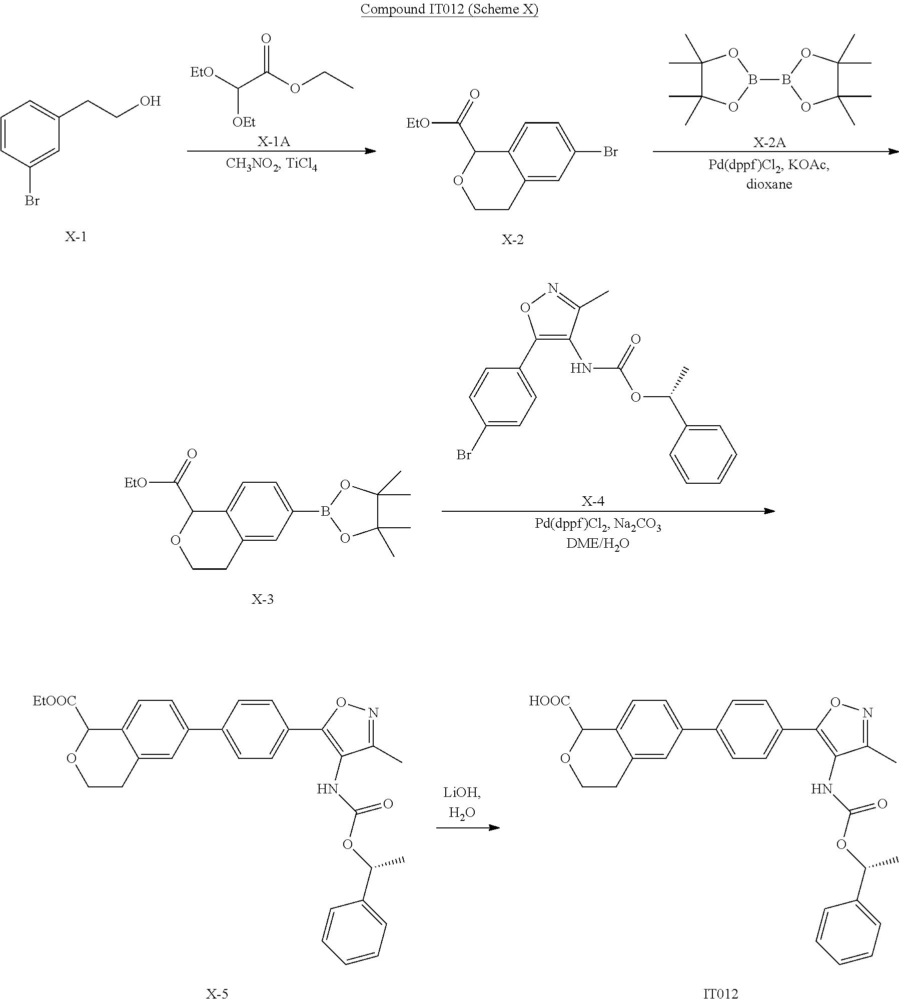

TiCl₄ (7.48 g, 40 mmol) was added over a period of 10 min to an ice-cooled mixture of X-1 (2 g, 10 mmol) and X-1A (2.12 g, 12 mmol) in CH₃NO₂ (20 mL). The solution was allowed to stir at rt for 12 hrs. Then the mixture was poured into the HCl (aq.1N) and extracted with DCM, dried over Na₂SO₄, concentrated in vacuo. The residue was purified by column chromatography to afford X-2 (0.5 g, yield 95%).

X-3 was prepared following the similar procedure described in the preparation of VI-3 with 67% yield. X-5 was prepared following the similar procedure described in the preparation of VIII-5 with 32% yield.

IT012 and its sodium salt IT012a were prepared following the similar procedure described in the preparation of IT001 and IT001a. IT012: MS (ESI) m/z (M+H)⁺ 499.2. IT012a: ¹H NMR (DMSO-d₆, 400 MHz): δ 7.75-7.78 (m, 2H), 7.73-7.74 (m, 3H), 7.38-7.42 (m, 4H), 7.31 (s, 1H), 5.74-5.76 (m, 1H), 4.78 (s, 1H), 4.38-4.41 (m, 1H), 3.71-3.74 (m, 1H), 2.76-2.79 (m, 2H), 2.11 (s, 3H), 1.50 (s, 3H). MS (ESI) m/z (M+H)⁺ 499.2.

### Example 5

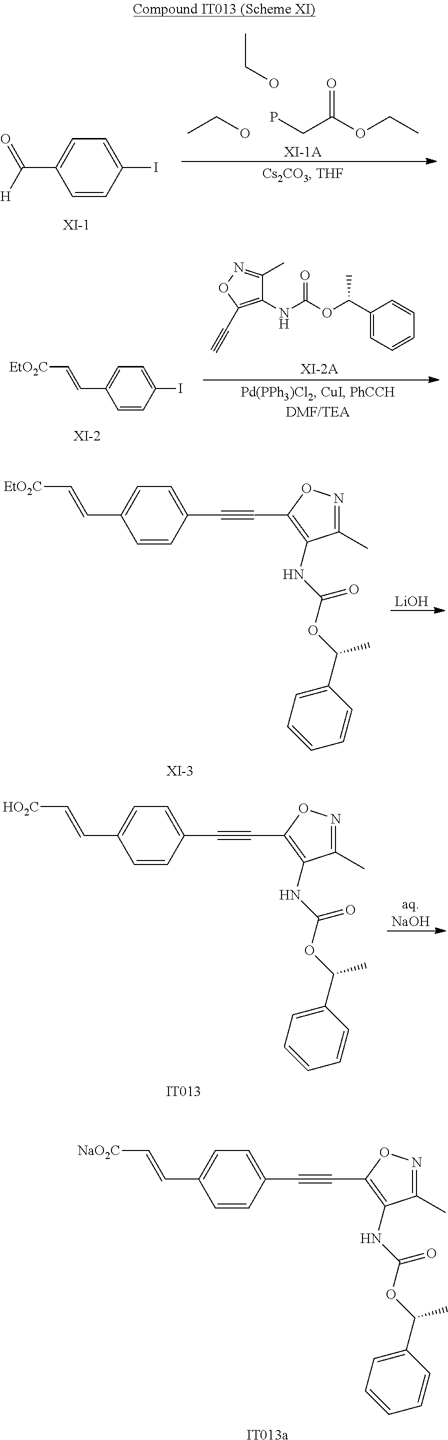

A solution of XI-1 (100 mg, 0.43 mmol), XI-1A (97 mg, 0.43 mmol) and Cs₂CO₃ (210 mg, 0.64 mmol) in 2 mL of THF was stirred overnight at rt. The mixture was treated with H₂O, and extracted with EtOAc. The combined organic layer was washed with brine, dried and concentrated. The residue was purified by prep-TLC (PE) to afford XI-2 (120 mg, yield 93.0%).

To a solution of XI-2 (130 mg, 0.43 mmol) in 2 mL of DMF/TEA (v/v=3/1), which was degassed by argon, was added Pd(PPh₃)₂Cl₂ (13 mg, 0.019 mmol) and phenyl acetylene (8 uL, 0.071 mmol). Then a solution of XI-2A (85 mg, 0.31 mmol) in 6 mL of DMF/TEA (v/v=3/1) was added dropwise. After stirred for 30 minutes, the mixture was diluted with H₂O, and extracted with EtOAc. The combined organic layer was washed with brine, dried and concentrated. The residue was purified by flash column chromatography over silica gel (PE/EA=6/1) to afford XI-3 (91 mg, yield 66%).

IT013 and its sodium salt IT013a were prepared following the similar procedure described in the preparation of IT001 and IT001a. IT013: ¹H NMR (400 MHz, CDCl₃): δ 7.66-7.72 (m, 3H), 7.51 (d, J=8.0 Hz, 2H), 7.30-7.42 (m, 5H), 6.58 (d, J=16 Hz, 1H), 5.85 (q, 1H), 2.22 (s, 3H), 1.59 (d, J=6.4 Hz, 3H). MS (ESI) m/z (M+H)⁺ 417.1. IT013a: ¹H NMR (DMSO-d₆, 400 MHz): δ 9.55 (br, 1H), 7.53 (d, J=8.4 Hz, 2H), 7.30-7.41 (m, 7H), 7.05 (d, J=16 Hz, 1H), 6.44 (d, J=16 Hz, 1H), 5.77 (q, 1H), 2.151 (s, 3H), 1.51 (d, J=6.4 Hz, 3H). MS (ESI) m/z (M+H)⁺ 417.1.

### Example 6-A

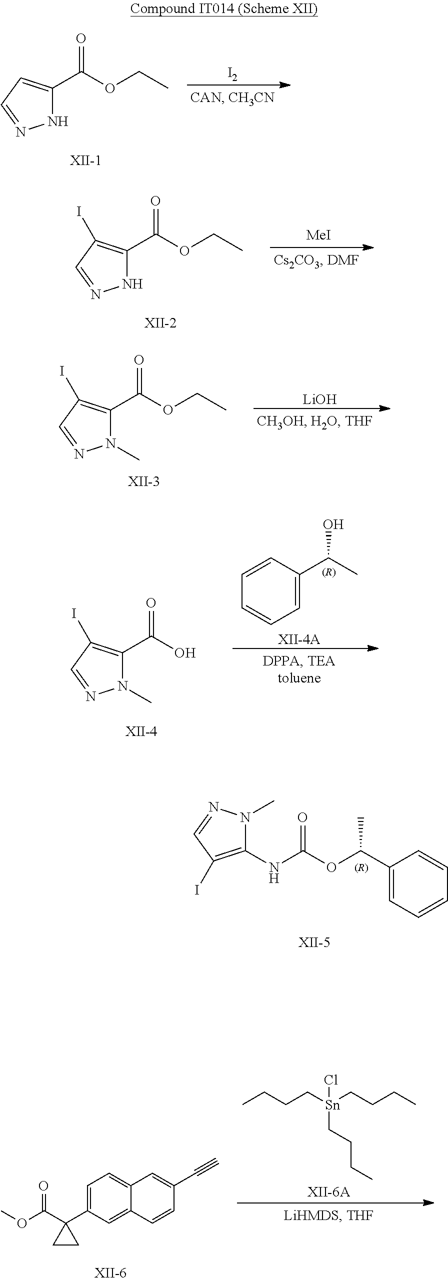

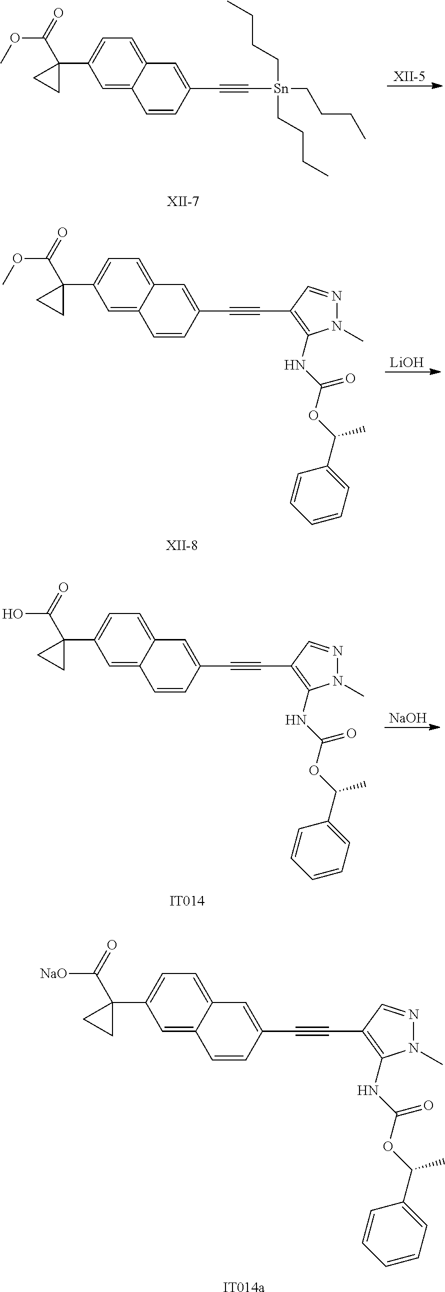

XII-6 was prepared from 2-(6-bromonaphthalen-2-yl)acetonitrile in five-step reactions.

To a solution of XII-1 (10 g, 71.4 mmol) in CH₃CN (182 mL) was added CAN (39.1 g, 71.4 mmol). The mixture was stirred at 25° C. for 15 min. Then I₂ (18 g, 71.4 mmol) was added. The mixture was stirred at 25° C. for 12 h. Then the mixture was quenched with 5% cold aq. NaHSO₃, until the solution turned into light yellow. The solid was filtered. The filtrate was extracted with EtOAc. The organics were collected, dried with Na₂SO₄, filtered, and concentrated. The residue was purified by column (PE:EA=3:1) to afford XII-2 (7.8 g, yield: 41%).

To a solution of XII-2 (8 g, 30.07 mmol) in DMF (150 mL) was added Cs₂CO₃ (29.3 g, 90 mmol) and CH₃I (10.6 g, 75.2 mmol). The mixture was stirred at 25° C. for 12 h. Then the mixture was washed with H₂O, and extracted with EtOAc. The organics were combined, dried with Na₂SO₄, filtered, and concentrated under reduced pressure. The residue was purified by column chromatography (PE:EA=10:1) to afford XII-3 (4 g, yield: 47.5%).

To a stirred solution of XII-3 (700 mg, 2.5 mmol) in 15 mL of MeOH/H₂O/THF (v/v/v=1/1/1) was added lithium hydroxide monohydrate (1.05 mg, 25 mmol). After the addition, the solution was stirred at 25° C. for 2 h. The mixture was concentrated in vacuo and adjusted pH to 4 with HCl (1N). The aqueous phase was extracted with EtOAc. The combined organic layer was washed with brine, dried over Na₂SO₄, and concentrated to afford crude XII-4 (623.0 mg, crude yield 98%), which was used to next step directly.

The mixture of XII-4 (2.1 g, 8.3 mmol), XII-4A (1.2 g, 9.8 mmol), DPPA (2.7 g, 9.8 mmol) and TEA (1.68 g, 16.6 mmol) in toluene (20 mL) was stirred at 80° C. under nitrogen for 2 hrs. Then the mixture was washed with H₂O, and extracted with EtOAc. The organics were combined, dried with Na₂SO₄, filtered, and concentrated under reduced pressure. The residue was purified by column chromatography on silica gel (PE:EA=3:1) to afford XII-5 (2.3 g, yield: 74.4%).

To a mixture of XII-6 (1.0 g, 4.0 mmol) in THF (10 mL) was added LiHMDS (4.8 mL, 4.8 mmol) at −78° C. The reaction mixture was stirred for 1 h at −78° C. and then XII-6A (2.60 g, 4.8 mmol) was added. The reaction mixture was stirred overnight and quenched with satur. NH4Cl (10 mL). The mixture was extracted with EtOAc, and the combined organic layer was washed with brine, dried over Na2SO4, and concentrated. The residue was purified by chromatography on silica gel (PE/EA=4:1) to afford XII-7 (410 mg, yield: 18.6%).

To a mixture of XII-7 (335.6 mg, 0.622 mmol), PPh₃ (18.7 mg, 0.072 mmol) and XII-5 (210 mg, 0.566 mmol) in THF (10 mL) was added Pd(OAc)₂ (7.9 mg, 0.036 mmol) under Ar at rt. The reaction mixture was heated at 50° C. for 2 hrs and then diluted with water. The mixture was extracted with EtOAc, and the combined organic layer was washed with brine, dried over Na₂SO₄, concentrated to get XII-8 (150 mg, crude yield: 49.0%), which was used directly without further purification.

IT014 and its sodium salt IT014a were prepared following the similar procedure described in the preparation of IT001 and IT001a. IT014: MS (ESI) m/z (M+H)⁺ 480.0. IT014a: ¹H NMR (DMSO-d₆, 400 MHz) δ 7.85 (s, 1H), 7.63-7.75 (m, 4H), 7.28-7.38 (m, 7H), 5.78-5.80 (q, 1H), 3.61 (s, 3H), 1.50-1.52 (d, J=6.0 Hz, 3H), 1.21 (brs, 2H), 0.77 (brs, 2H). MS (ESI) m/z (M+H)⁺480.0.

IT015 was prepared following the similar synthetic route for the preparation of IT014 using ethyl 1-cyclopropyl-4-iodo-1H-pyrazole-5-carboxylate (XII-3A) in place of XII-3. Preparation of XII-3A: To a solution of XII-2 (6.8 g, 25.5 mmol) in 1,4-dioxane (200 mL) was added Cu(OAc)₂ (3.9 g, 21.4 mmol), Cs₂CO₃ (20.7 g, 63.5 mmol), DMAP (12.5 g, 102.5 mmol) and cyclopropylboronic acid (4.39 g, 51.04 mmol). The mixture was stirred at 50° C. for 12 h. The solvent was removed under reduced pressure. Then the mixture was washed with H₂O, extracted with EtOAc. The organics were combined, dried with Na₂SO₄, filtered, and concentrated. The residue was purified by column (PE:EA=30:1) to afford XII-3A (2.2 g, yield: 28.2%). IT015: MS (ESI) m/z (M+H)⁺ 506.2.

Sodium salt IT015a was prepared following the similar procedure described in the preparation of IT001a. ¹H NMR (DMSO-d₆, 400 MHz): δ 7.86 (s, 1H), 7.76 (d, J=8.4 Hz, 1H), 7.65-7.76 (m, 3H), 7.52 (d, J=8.4 Hz, 1H), 7.29-7.39 (m, 6H), 5.79-5.83 (m, 1H), 3.39-3.41 (m, 1H), 1.52 (d, J=5.2 Hz, 3H), 1.20-1.21 (br, 2H), 0.89-0.90 (m, 4H), 0.75 (br, 2H). MS (ESI) m/z (M+H)⁺506.2.

IT016 was prepared following the similar synthetic route for the preparation of IT014 using ethyl 1-ethyl-4-iodo-1H-pyrazole-5-carboxylate (XII-3B) in place of XII-3. XII-3B was prepared following the similar procedure for the synthesis of XII-3 using C₂H₅I in place of CH₃I. IT016: MS(ESI) m/z (M+H)⁺ 494.2.

Sodium salt IT016a was prepared following the similar procedure described in the preparation of IT001a. ¹H NMR (DMSO-d₆, 400 MHz) δ : 7.84 (s, 1H), 7.76 (d, J=8.4 Hz, 1H), 7.63-7.67 (m, 3H), 7.53 (d, J=8.4 Hz, 1H), 7.21-7.37 (m, 6H), 5.78-5.80 (q, 1H), 3.91-3.96 (q, 2H), 1.50 (d, J=6.0 Hz, 3H), 1.23-1.27 (m, 5H), 0.77 (br, 2H). MS (ESI) m/z (M+H)⁺ 494.2.

### Example 6-B

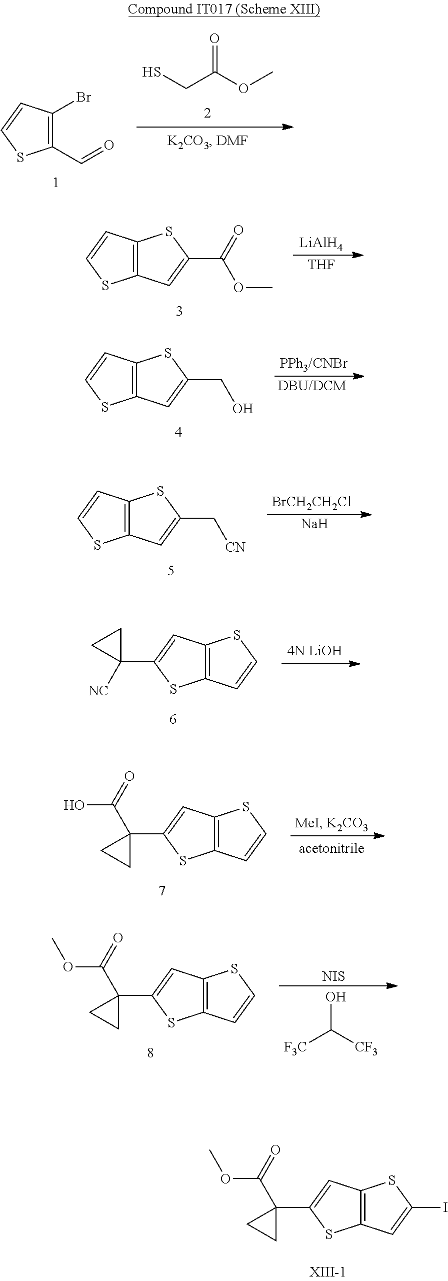

To a mixture of compound 1 (350 g, 1.83 mol) and K₂CO₃ (1000 g, 7.33 mol) in DMF (4000 mL) was added compound 2 (195 g, 1.83 mol) at rt. The resultant mixture was stirred at 70° C. for 5 hs. After cooled to rt, the mixture was poured into ice-water and solids were precipitated out which was obtained by filtration and dried in vacuo at 50° C. to give compound 3 (300 g, 82.6%) as a white solid.

Under nitrogen, compound 3 (300 g, 1.52 mol) dissolved in anhydrous THF (2500 mL) was added dropwise to a mixture of LiAlH₄ (75 g, 1.98 mol) in anhydrous THF (1500 mL) at 0° C. After the addition, the mixture was stirred at rt for 2 hs and compound 3 was consumed completely. Cooled to 0° C., water (75 mL) was added dropwise followed by the addition of 10% NaOH aq. (125 mL) dropwise. The mixture was filtered and the cake was washed with DCM several times. The filtration was concentrated under reduced pressure to give compound 4 (258 g, 77.6%) as a white solid.

Under nitrogen, PPh₃ (415.2 g, 1.58 mol) dissolved in anhydrous DCM (1000 mL) was added to BrCN (183 g, 1.73 mol) at 0° C., followed by the addition of compound 4 (245 g, 1.44 mol) dissolved in anhydrous DCM (3000 mL). The resultant solution was stirred at rt until compound 4 was consumed completely and then the solution was cooled to 0° C. and DBU (285 g, 1.87 mol) was added dropwise. After the addition, the solution was stirred at rt for 16 hs. The solvent was removed under reduced pressure to give the residue which was purified by silica gel column chromatography (PE/EA=20:1) to afford compound 5 (150 g, 58.15%) as a yellow solid.

Under nitrogen, to a mixture of NaH (60%, 56 g, 1.4 mol) in anhydrous THF (500 mL) was added a solution of compound 5 (100 g, 0.56 mol) in anhydrous THF (500 mL) dropwise at 0° C. The mixture was stirred at 0° C. for 1 h, followed by the addition of 1-bromo-2-chloroethane (120 g, 0.84 mol) at 0° C. and the mixture was stirred at rt for 5 hs. Quenched with water, the mixture was diluted with water, extracted with EA, dried over Na2SO4, filtered and concentrated to give the residue which was purified by silica gel column chromatography (PE/EA=20:1) to afford compound 6 (203 g, 89.5%) as a yellow solid.

To a solution of LiOH in water (4N, 300 mL) was added compound 6 (60 g, 0.292 mmol) and the mixture was heated to reflux for 16 hs. After cooled to rt, the solution was extracted with DCM twice and the aqueous phase was acidified to pH˜2 with conc. HCl. The precipitate was collected by filtration, washed with water and dried in vacuo to give compound 7 (57 g, 82%) as a white solid.

To a mixture of compound 7 (400 g, 1.78 mol) and K₂CO₃ (493 g, 3.57 mol) in acetonitrile (4000 mL) was added CH₃I (304 g, 2.15 mol). The resultant mixture was heated to reflux for 16 hs. After cooled to rt, the mixture was filtered and the filtration was concentrated to give compound 8 (370 g, 87%).

To a solution of compound 8 (220 g, 0.923 mol) and 2,6-dimethylpyridine (99 g, 0.923 mol) in 1,1,1,3,3,3-hexafluoropropan-2-ol (2000 g) was added NIS (229 g, 1.02 mol) at rt. The reaction mixture was stirred at rt overnight. LCMS showed the reaction was completed, and then the mixture was diluted with water and extracted with EtOAc. The combined organic layers were washed with brine, and concentrated under reduced pressure. The residue was triturated with EA to give XIII-1 (310 g, 92%) as a pale solid.

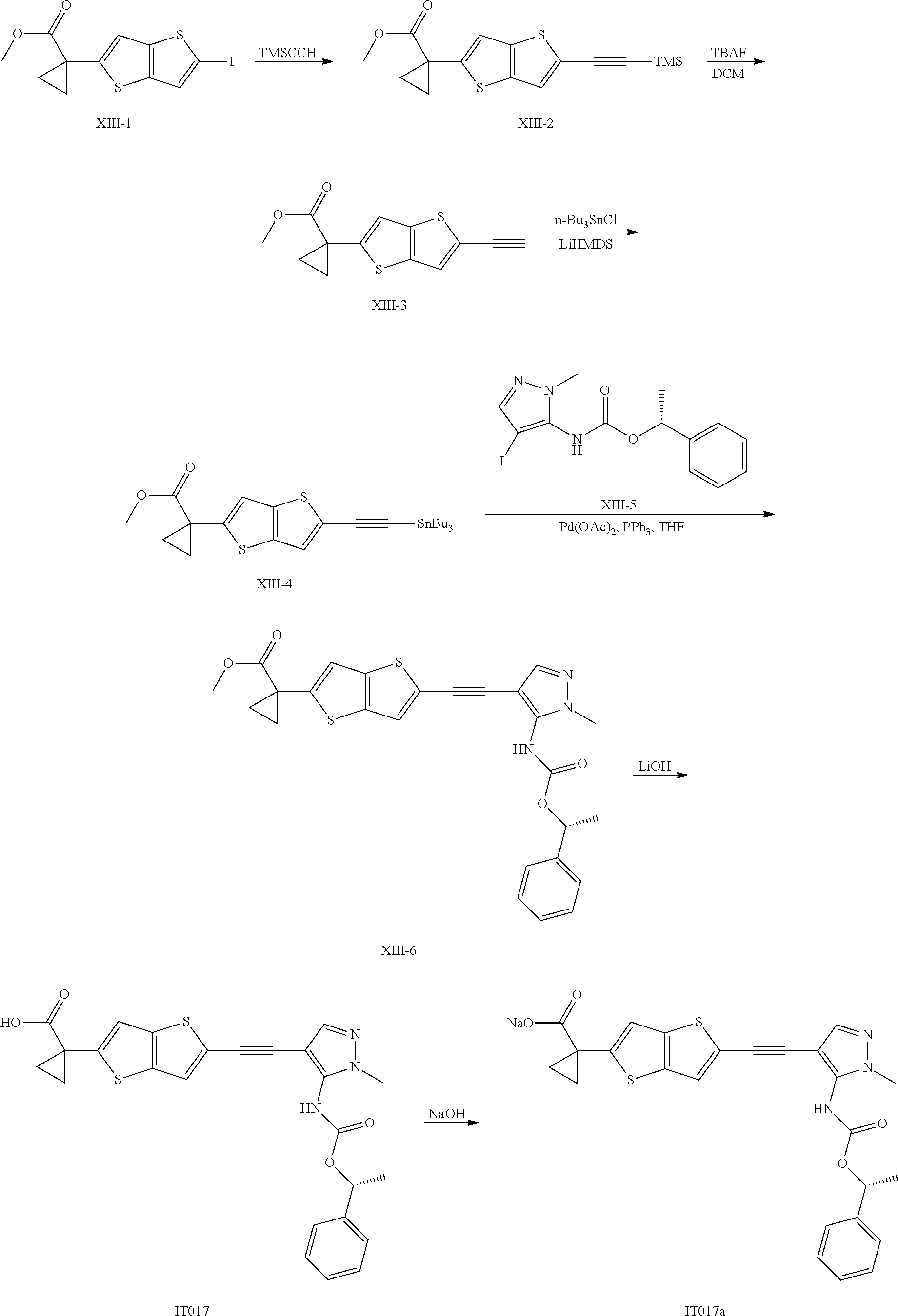

To a mixture of XIII-1 (2.0 g, 5.49 mmol), CuI (25.3 mg, 0.27 mmol) and Pd(PPh)₂Cl₂ (192 mg, 0.27 mmol) in DME/TEA (50 mL, v/v=3:1) was added TMSCCH (1.62 g, 16.48 mmol). The reaction mixture was stirred for 2 h and diluted with water (50 mL). The mixture was extracted with EA, and the combined organic layer was washed with brine, dried over Na₂SO₄, concentrated to afford crude XIII-2 (1.50 g, yield: 81.8%), which was used directly without further purification.

To a mixture of XIII-2 (1.50 g, 4.5 mmol) in DCM (30 mL) was added TBAF (2.70 g, 11.25 mmol). The reaction mixture was stirred for 2 h and diluted with water. The mixture was extracted with DCM, and the combined organic layer was washed with brine, dried over Na₂SO₄, concentrated. The residue was purified by chromatography on silica gel (PE:EA=10:1) to afford XIII-3 (780 mg, yield: 66.1%).

To a mixture of XIII-3 (780 mg, 2.96 mmol) in THF (10 mL) was added LiHMDS (8.8 mL, 8.8 mmol) at −78° C. The reaction mixture was stirred for 1 h at −78° C. and n-Bu₃SnCl (3.0 g, 9.23 mmol) was added. The reaction mixture was stirred for overnight and quenched with sat. NH₄Cl (10 mL). The mixture was extracted with EA, and the combined organic layer was washed with brine, dried over Na₂SO₄, and concentrated to afford crude XIII-4 (1.42 g, yield: 89.3%), which was used directly without further purification.

To a mixture of XIII-4 (334 mg, 0.61 mmol), PPh₃ (17.3 mg, 0.061 mmol) and XIII-5 (225 mg, 0.61 mmol) in THF (10 mL) was added Pd(OAc)₂ (7.3 mg, 0.03 mmol) under Argon at rt. The reaction mixture was heated at 50° C. for 2 h and then diluted with water (20 mL). The mixture was extracted with EtOAc, and the combined organic layer was washed with brine, dried over Na₂SO₄, and concentrated to get crude product, which was purified by prep-HPLC to afford XIII-6 (98 mg, yield: 32.5%).

IT017 and its sodium salt IT017a were prepared following the similar procedure described in the preparation of IT001 and IT001a. IT017a: ¹H NMR (DMSO-d₆, 400 MHz): δ 7.60 (s, 1H), 7.26-7.37 (m, 6H), 6.81 (s, 1H), 5.74-5.76 (q, 1H), 3.57 (s, 3H), 1.48-1.50 (d, J=6.4 Hz, 3H), 1.43 (br, 2H), 0.94 (br, 2H) MS (ESI) m/z (M+H)⁺ 492.1.

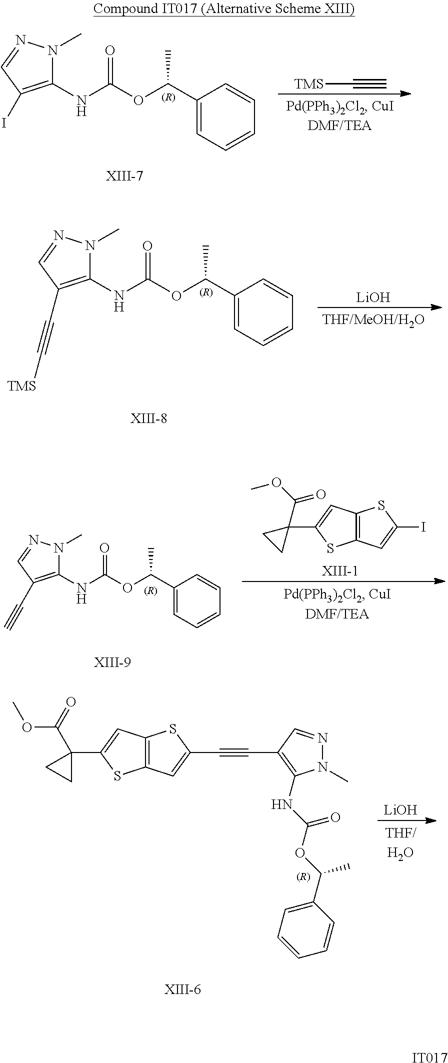

XIII-7 was prepared following the same procedure for the synthesis of XII-5.

The mixture of XIII-7 (500 mg, 1.35 mmol), ethynyltrimethylsilane (264 mg, 2.7 mmol), Pd(PPh₃)₂Cl₂ (94.45 mg, 0.135 mmol) and CuI (25.65 mg, 0.135 mmol) in DMF/Et₃N (20 mL, v/v=3:1) was stirred at rt under nitrogen for 2 h. After concentrated, the residue was partitioned between H₂O and DCM. The aqueous phase was extracted with DCM, and the combined organic layer was washed with brine, dried over Na₂SO₄, concentrated. The residue was purified by chromatography on silica gel (PE:EA=4:1) to afford XIII-8 (400 mg, yield 86.96%).

To a solution of XIII-8 (400 mg, 1.17 mmol) in MeOH (2 mL), THF (2 mL) and H₂O (3 mL), was added LiOH.H₂O (245.6 mg, 5.85 mmol). The reaction mixture was stirred at rt for 2 h. After concentrated, the residue was partitioned between H₂O and EA, the aqueous phase was extracted with EA, and the combined organic layer was washed with brine, dried over Na₂SO₄, concentrated. The residue was purified by chromatography on silica gel (PE:EA=4:1) to afford XIII-9 (220 mg, yield: 70.29%).

To a mixture of XIII-1 (379 mg, 1.04 mmol), CuI (14.06 mg, 0.074 mmol) and Pd (PPh₃)₂Cl₂ (52.11 mg, 0.074 mmol) in DMF/TEA (4 mL, v/v=1/3) was added PhCCH (1.02 mg, 0.01 mmol). The reaction mixture was stirred for 2 min and then XIII-9 (200 mg, 0.74 mmol, in DMF/TEA) was added. The reaction mixture was stirred for 2 h and diluted with water. The mixture was extracted with DCM, and the combined organic layer was washed with brine, dried over Na₂SO₄, concentrated. The residue was purified by chromatography on silica gel (PE:EA=4:1) to afford XIII-6 (330 mg, yield: 88%).

IT017 and its sodium salt IT017a were prepared following the similar procedure described in the preparation of IT001 and IT001a. IT017: MS (ESI) m/z (M+H)⁺ 492.2. IT017a: ¹H NMR (DMSO-d₆, 400 MHz) δ 9.83 (s, 1H), 7.65 (s, 1H), 7.33-7.35 (m, 6H), 6.84 (s, 1H), 5.75-5.80 (q, 1H), 3.60 (s, 2H), 1.51 (d, J=6.4 Hz, 3H), 1.50 (br, 2H), 0.98 (br, 2H). MS (ESI) m/z (M+H)⁺ 492.2.

Preparation of potassium salt IT017b: To a solution of IT017 (120 mg, 0.244 mmol) in MeOH (10 mL) was added drop wise a solution of aq. KOH (13.65 mg, 0.244 mmol). The mixture was stirred at rt for 30 min. Then the mixture was concentrated and freeze-dried under vacuum. The product was obtained as potassium salt without further purification. MS (ESI) m/z (M+H)⁺ 492.2. ¹H NMR (DMSO-d₆, 400 MHz): δ 9.80 (s, 1H), 7.66 (s, 1H), 7.27-7.35 (m, 6H), 6.83 (s, 1H), 5.74-5.77 (m, 1H), 3.60 (s, 3H), 1.50 (d, J=6.4 Hz, 3H), 1.41 (br, 2H), 1.06 (br, 2H).

Preparation of caldium salt IT017c: To a solution of IT017 (200 mg, 0.41 mmol) in MeOH (10 mL) and water (2 mL) was added Ca(OH)₂ (15 mg, 0.205 mmol) portion wise. The mixture was heated at 60° C. for 1 h. Then the mixture was concentrated and freeze-dried under vacuum. The product was obtained as calcium salt without further purification. MS (ESI) m/z (M+H)⁺ 492.2. ¹H NMR (DMSO-d₆, 400 MHz): δ 9.80 (s, 1H), 7.66 (s, 1H), 7.27-7.35 (m, 6H), 6.83 (s, 1H), 5.73-5.76 (m, 1H), 3.59 (s, 3H), 1.49-1.51 (m, 5H), 1.06 (br, 2H).

Preparation of trisamine salt IT017d: To a solution of IT017 (200 mg, 0.407 mmol) in MeOH (10 mL) and water (2 mL) was added trisamine(2-Amino-2-hydroxymethyl-propane-1,3-diol) (49.18 mg, 0.407 mmol) portion wise. The mixture was heated at 60° C. for 1 h. Then the mixture was concentrated and freeze-dried under vacuum. The product was obtained as trisamine salt without further purification. MS (ESI) m/z (M+H)⁺ 492.2. ¹H NMR (DMSO-d₆, 400 MHz): δ 7.60 (s, 1H), 7.30-7.33 (m, 6H), 7.04 (s, 1H), 5.84-5.89 (m, 1H), 3.69 (s, 3H), 3.65 (s, 6H), 1.59-1.61 (m, 5H), 1.19-1.20 (m, 2H).

IT047 was prepared by reacting XIII-1 with the corresponding acetylene (R)-1-(2-chlorophenyl)ethyl (5-ethynyl-3-methylisoxazol-4-yl)carbamate following the similar procedure in the preparation of 1-6, followed by the standared LiOH hydrolysis and NaOH basification. IT047: MS (ESI) m/z (M+H)⁺ 527.2. IT047a: ¹H NMR (400 MHz, DMSO-d₆): δ 9.64 (s, 1H), 7.63 (s, 1H), 7.39-7.52 (m, 2H), 7.32-7.37 (m, 2H), 6.89 (s, 1H), 5.99-6.04 (q, 1H), 2.14 (s, 3H), 1.50-1.52 (m, 5H), 1.01 (br, 2H). MS (ESI) m/z (M+H)⁺ 527.0.

IT048 was prepared by reacting XIII-1 with the corresponding acetylene benzyl (5-ethynyl-3-methylisoxazol-4-yl)carbamate following the similar procedure in the preparation of I-6, followed by the standared LiOH hydrolysis and NaOH basification. Sodium salt IT048a: ¹H NMR (400 MHz, DMSO-d₆): δ 7.75 (s, 1H), 7.33-7.41 (m, 5H), 6.86 (s, 1H), 5.15 (s, 2H), 2.11 (s, 3H), 1.47-1.52 (m, 2H), 0.93-1.04 (m, 2H). MS (ESI) m/z (M+H)⁺ 479.1.

IT070 was prepared by following the similar alternative synthetic scheme XIII of IT017 using (R)-1-phenylethyl (1-ethyl-4-ethynyl-1H-pyrazol-5-yl)carbamate in place of XIII-9. MS (ESI) m/z (M+H)⁺ 506.0. Sodium salt IT070a: ¹H NMR (DMSO-d₆, 400 MHz): δ 9.70 (s, 1H), 7.69 (s, 1H), 7.26-7.34 (m, 6H), 6.86 (s, 1H), 5.78 (q, J=6.4 Hz, 1H), 3.94 (q, J=6.8 Hz, 2H), 1.46-1.51 (m, 5H), 1.24 (t, J=7.6 Hz, 3H), 1.00 (br, 2H). MS (ESI) m/z (M+H)⁺ 506.0.

IT106 was prepared by following the similar alternative synthetic scheme XIII of IT017 using (R)-1-phenylethyl (3-ethynylpyridin-4-yl)carbamate in place of XIII-9. ¹HNMR (DMSO-d₆, 400 MHz): δ 12.81 (s, 1H), 9.67 (s, 1H), 8.63 (s, 1H), 8.42 (s, 1H), 7.82 (s, 1H), 7.80 (s, 1H), 7.32-7.73 (m, 6H), 5.86-5.91 (m, 1H), 1.65 (m, 2H), 1.57 (m, 3H), 1.41 (m, 2H). MS (ESI) m/z (M+H)⁺ 488.9.

IT107 was prepared by following the similar alternative synthetic scheme XIII of IT017 using methyl 1-(5-iodo-3-methylthieno[3,2-b]thiophen-2-yl)cyclopropanecarboxylate in place of XIII-1. ¹H NMR (400 MHz, Methanol-d₄): δ 7.58 (s, 1H), β 7.38 (br, 2H), 7.30-7.33 (m, 2H), 7.24-7.27 (m, 1H), 7.19 (s, 1H), 5.82-5.87 (q, J=6.4 Hz, 1H), 3.67 (s, 3H), 2.28 (s, 3H), 1.60-1.63 (m, 2H), 1.57 (d, J=6.4 Hz, 3H), 1.14-1.16 (m, 2H). MS (ESI) m/z (M+H)⁺ 506.0.

IT108 was prepared by following the similar alternative synthetic scheme XIII of IT017 using (R)-1-phenylethyl (4-ethynylisothiazol-5-yl)carbamate in place of XIII-9. MS (ESI) m/z (M+H)⁺ 495.1. ¹H NMR (Methanol-d₄, 400 MHz): δ 8.20 (s, 1H), 7.47 (s, 1H), 7.41-7.43 (m, 2H), 7.33-7.37 (m, 2H), 7.28-7.30 (m, 1H), 7.15 (s, 1H), 5.89-5.94 (m, 1H), 1.70-1.73 (m, 2H), 1.61-1.63 (d, J=6.4 Hz, 3H), 1.40-1.42 (m, 2H).

IT109: (R)-1-(4-ethynyl-1-methyl-1H-pyrazol-5-yl)-3-(1-phenylethyl)urea was first prepared by reacting XII-4 with (R)-1-phenylethanamine following the similar procedure described in the synthesis of XII-5 in Example 6-A. IT109 was prepared by following the similar alternative synthetic scheme XIII of IT017 using (R)-1-(4-ethynyl-1-methyl-1H-pyrazol-5-yl)-3-(1-phenylethyl)urea in place of XIII-9. MS (ESI) m/z (M+H)⁺ 491.0.

Sodium salt IT109a: ¹H NMR (DMSO-d₆, 400 MHz): δ 10.44 (s, 1H), 8.82 (s, 1H), 7.58 (s, 1H), 7.38-7.40 (d, J=7.2 Hz, 3H), 7.27-7.31 (t, J=7.6 Hz, 2H), 7.17-7.20 (t, J=7.2 Hz, 1H), 6.86 (s, 1H), 4.78-4.85 (m, 1H), 3.55 (s, 3H), 1.51-1.52 (d, J=5.2 Hz, 2H), 1.36-1.38 (d, J=6.8 Hz, 3H), 1.02-1.03 (m, 2H). MS (ESI) m/z (M+H)⁺ 491.0.

IT110 was prepared by following the similar alternative synthetic scheme XIII of IT017 using methyl 1-(5-iodo-3,6-dimethylthieno[3,2-b]thiophen-2-yl)cyclopropanecarboxylate in place of XIII-1. ¹H NMR (400 MHz, Methanol-d₄): δ 7.60 (s, 1H), 67.40 (d, J=6.8 Hz, 2H), 7.33-7.25 (m, 3H), 5.87 (q, J=6.4 Hz, 1H), 3.70 (s, 3H), 2.30 (d, J=5.2 Hz, 6H), 1.60 (m, 5H), 1.10-1.12 (m, J=2.8 Hz, 2H). MS (ESI) m/z (M+H)⁺ 520.0.

IT114 was prepared by following the similar alternative synthetic scheme XIII of IT017 using (S)-2,2,2-trifluoro-1-phenylethyl (4-ethynyl-1-methyl-1H-pyrazol-5-yl)carbamate in place of XIII-9. ¹H NMR (DMSO-d₆, 400 MHz): δ 9.997 (s, 1H), 7.65 (s, 1H), 7.54 (s, 2H), 7.44-7.46 (t, J=6.4 Hz, 3H), 7.25 (s, 1H), 6.30-6.36 (m, 1H), 3.66 (s, 3H), 1.65-1.68 (m, 2H), 1.38-1.39 (d, J=6.4 Hz, 2H). MS (ESI) m/z (M+H)⁺ 545.9.

IT115 was prepared by following the similar alternative synthetic scheme XIII of IT017 using (R)-1-phenylethyl (2-ethynylpyridin-3-yl)carbamate in place of XIII-9. ¹H NMR (DMSO-d₆, 400 MHz): δ 12.78 (br, 1H), δ 9.53 (s, 1H), 8.35-8.36 (m, 1H), 7.92 (s, 1H), 7.66 (s, 1H), 7.39-7.41 (m, 6H), 7.28-7.37 (m, 1H), 5.83 (t, J=6.4 Hz, 1H), 2.37 (m, 1H), 1.63-1.66 (m, 2H), 1.54 (d, J=6.8 Hz, 3H), 2.28 (s, 3H), 1.40-1.42 (m, 2H). MS (ESI) m/z (M+H)⁺ 489.0.

IT116 was prepared by following the similar alternative synthetic scheme XIII of IT017 using (R)-1-phenylethyl (4-ethynylthiazol-5-yl)carbamate in place of XIII-9. MS (ESI) m/z (M+H)⁺ 495.1. Sodium salt IT116a: ¹H NMR (DMSO-d₆, 400 MHz): δ 9.50 (s, 1H), 7.50 (s, 1H), 7.49-7.45 (m, 2H), 7.40-7.36 (m, 2H), 7.32-7.30 (m, 2H), 7.05 (s, 1H), 5.93-8.90 (m, 1H), 1.64-1.61 (m, 5H), 1.19 (m, 2H).

IT117 was prepared by the Suzuki-Coupling of ethyl 1-ethynylcyclopropanecarboxylate with (R)-1-phenylethyl (5-(5-bromothieno[3,2-b]thiophen-2-yl)-3-methylisoxazol-4-yl)carbamate using the similar procedure in the synthesis of XIII-6, followed by standard LiOH hydrolysis. The carbamate intermediate was prepared following the similar procedure for the synthesis of X3 in Example 41. ¹H NMR (Methanol-d₄, 400 MHz): δ 7.54 (s, 1H), 7.35-7.46 (m, 5H), 7.54 (br, 1H), 7.66 (d, J=6.4 Hz, 1H), 2.17 (s, 3H), 1.61-1.66 (m, 5H), 1.43 (q, J=6.4 Hz, 2H). MS (ESI) m/z (M+H)⁺ 493.1.

IT118 was prepared by following the similar alternative synthetic scheme XIII of IT017 using (R)-1-(perfluorophenyl)ethyl (4-ethynyl-1-methyl-1H-pyrazol-5-yl)carbamate in place of XIII-9. ¹H NMR (DMSO-d₆, 400 MHz): δ 9.96 (s, 1H), 7.65 (s, 1H), 7.46 (s, 1H), 7.24 (s, 1H), 5.98-6.03 (d, J=6.8 Hz, 1H), 3.59 (s, 3H), 1.62-1.60 (m, 5H), 1.36-1.37 (m, 2H). MS (ESI) m/z (M+H)⁺ 581.9.

IT125 was prepared by following the similar alternative synthetic scheme XIII of IT017 using (R)-1-phenylethyl (4-ethynyloxazol-5-yl)carbamate in place of XIII-9. ¹H NMR (Methanol-d₄, 400 MHz): δ 8.06 (s, 1H), 7.40-7.42 (d, J=8.0 Hz, 2H), 7.33-7.36 (m, 3H), 7.28-7.30 (d, J=8.0 Hz, 1H), 7.20 (s, 1H), 5.85-5.87 (m, 1H), 1.76 (s, 2H), 1.59-1.60 (d, J=6.4 Hz, 3H), 1.46 (s, 2H). MS (ESI) m/z (M+H)⁺ 479.1.

IT127 was prepared by following the similar alternative synthetic scheme XIII of IT017 using (R)-1-phenylethyl (4-ethynyl-1-((2-(trimethylsilyl)ethoxy)methyl)-1H-pyrazol-5-yl)carbamate in place of XIII-9. After Suzuki-coupling, SEM protecting group was removed by LiBF₄ in acetonitrile at 80° C. for 10 h followed by standard LiOH hydrolysis to afford IT127 as the final product. MS (ESI) m/z (M+H)⁺ 478.1. ¹H NMR (Methanol-d₄, 400 MHz): δ 7.76 (s, 1H), 7.35-7.44 (m, 2H), 7.27-7.34 (m, 3H), 7.21 (s, 1H), 7.03 (s, 1H), 5.86-5.88 (m, 1H), 1.59-1.60 (m, 5H), 1.17-1.19 (m, 2H).

IT145 was prepared by the Suzuki-Coupling of methyl 1-ethynylcyclopropanecarboxylate with intermediate X2 (disclosed in Example 41) using the similar procedure in the synthesis of XIII-6, followed by standard LiOH hydrolysis. ¹H NMR (400 MHz, DMSO-d₆): δ 12.95 (br, 1H), 10.02 (br, 1H), 7.59 (s, 1H), 7.34-7.42 (m, 6H), 5.76 (br, 1H), 3.83 (s, 3H), 1.51-1.55 (m, 5H), 1.41-1.42 (m, 2H). MS (ESI) m/z (M+H)⁺ 493.1.

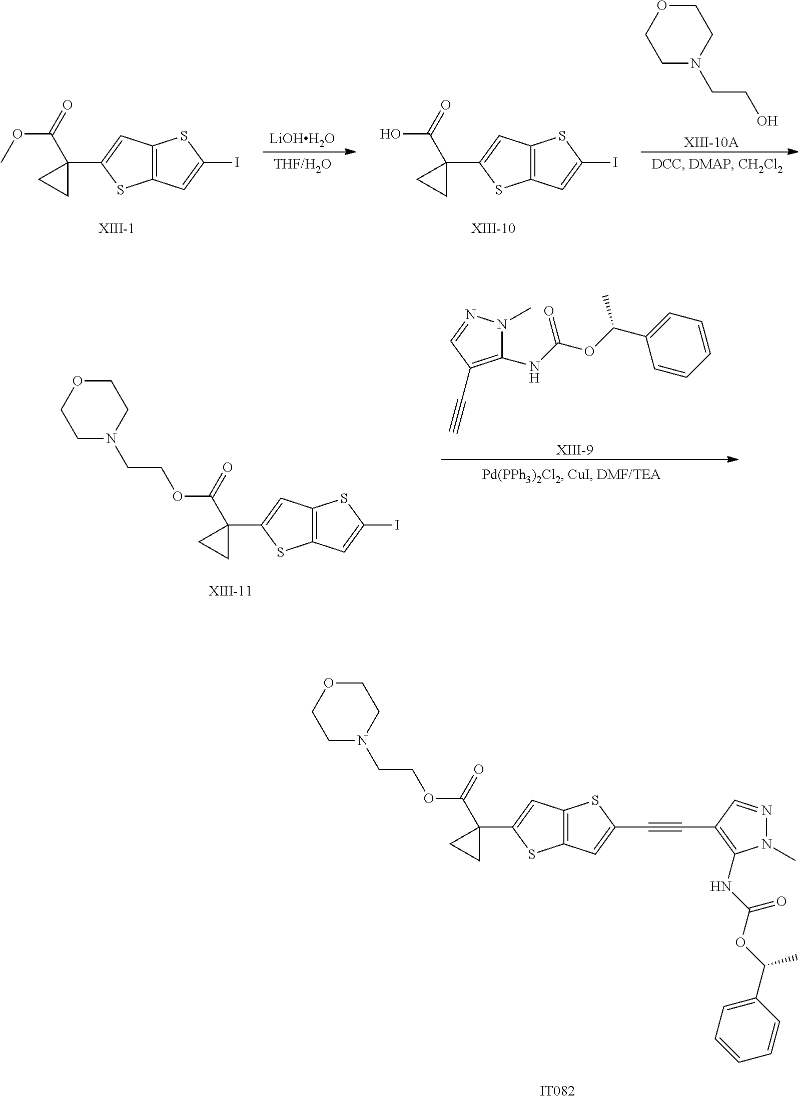

XIII-10 was obtained from XIII-1 by LiOH hydrolysis. To a solution of XIII-10 (100 mg, 0.286 mmol) in CH₂Cl₂ (3 mL) was added DCC (53 mg, 0.257 mmol) and DMAP (3.49 mg, 0.03 mmol). After 30 min, XIII-10A (37.4 mg, 0.286 mmol) was added. Then the mixture was stirred at 25° C. for 3 hrs. After concentrated, the mixture was diluted with EtOAc, washed with brine, dried over Na₂SO₄, filtered and concentrated. The residue was purified by column over silica gel (PE/EA=2/1) to afford XIII-11 (55 mg, yield 41.7%).

XIII-11 (55 mg, 0.12 mmol), Pd(PPh₃)₂Cl₂ (8.4 mg, 0.012 mmol), and CuI (2.3 mg, 0.012 mmol) were mixed with DMF (3 mL) and Et₃N (1 mL) under argon atmosphere. Then a solution of XIII-9 (35 mg, 0.13 mmol) in DMF (1.5 mL) and Et₃N (0.5 mL) was added slowly at rt. The mixture was stirred at rt for 2 hrs. Then the mixture was diluted with EtOAc, washed with brine, dried over Na₂SO₄, filtered and concentrated. The residue was purified by column over silica gel (DCM/MeOH=10/1) to afford IT082 (50 mg, yield 69.4%). ¹H NMR (400 MHz, DMSO-d₄): δ 9.79 (br, 1H), 7.68 (s, 1H), 7.46 (s, 1H), 7.27-7.36 (m, 6H), 5.77 (q, J=6.0 Hz, 1H), 4.14 (t, J=5.2 Hz, 2H), 3.61 (s, 1H), 3.43-3.46 (m, 4H), 2.46 (t, J=5.2 Hz, 2H), 2.28 (br, 4H), 1.63-1.66 (m, 2H), 1.50-1.52 (d, J=6.0 Hz, 3H), 1.44-1.45 (m, 2H). MS (ESI) m/z (M+H)⁺ 605.0.

IT083 was prepared by first hydrolying XIII-1 with NaBH₄ and CaCl₂ in EtOH to afford an intermediate alcohol, followed by Suzuki coupling with XIII-9 as described above in the synthesis of IT082. ¹H NMR (400 MHz, Methanol-d₄): δ 7.58 (s, 1H), 7.20-7.37 (m, 6H), 7.12 (s, 1H), 5.84 (q, J=6.0 Hz, 1H), 3.65-3.69 (m, 5H), 1.57 (d, J=6.0 Hz, 3H), 1.02 (brs, 4H). MS (ESI) m/z (M+H)⁺ 478.0.

IT084 was prepared by DCC coupling of XIII-10 with 2-methoxyethanol following the similar procedure described in the synthesis of XIII-11, followed by Suzuki Coupling with XIII-9 as described above in the synthesis of IT082. ¹H NMR (DMSO-d₆, 400 MHz): δ 9.81 (br, 1H), 7.70 (s, 1H), 7.48 (s, 1H), 7.29-7.38 (m, 6H), 5.80 (q, J=6.4 Hz, 1H), 4.19 (t, J=4.8 Hz, 2H), 3.63 (s, 1H), 3.50 (t, J=4.8 Hz, 2H), 3.22 (s, 3H), 1.67-1.70 (m, 2H), 1.53 (d, J=6.4 Hz, 3H), 1.47-1.49 (m, 2H). MS (ESI) m/z (M+H)⁺ 550.0.

IT085 was prepared by reacting XIII-10 with ethyl iodide to form the corresponding ethyl ester, then Suzuki Coupling with XIII-9 as described above in the synthesis of IT082. ¹H NMR (CDCl₃, 400 MHz): δ 7.60 (s, 1H), 7.31-7.35 (m, 5H), 7.24 (s, 1H), 7.06 (s, 1H), 6.43 (s, 1H), 5.90 (q, J=6.8 Hz, 1H), 4.18 (q, J=7.2 Hz, 2H), 3.73 (s, 3H), 1.74-1.77 (m, 2H), 1.59-1.62 (m, 5H), 1.39-1.40 (m, 2H), 1.24 (t, J=7.2 Hz, 3H). MS (ESI) m/z (M+H)⁺ 520.0.

IT086 was prepared by reacting XIII-10 with isopropyl iodide to form the corresponding isopropyl ester, then Suzuki Coupling with XIII-9 as described above in the synthesis of IT082. ¹H NMR (CDCl₃, 400 MHz): δ 7.61 (s, 1H), 7.30-7.38 (m, 5H), 7.24 (s, 1H), 7.04 (s, 1H), 6.44 (br, 1H), 5.90 (q, J=6.8 Hz, 1H), 4.99-5.06 (m, 1H), 3.73 (s, 3H), 1.72-1.75 (m, 2H), 1.59-1.62 (m, 3H), 1.37-1.39 (m, 2H), 1.21-1.23 (m, 6H). MS (ESI) m/z (M+H)⁺ 534.0.

IT087 was prepared by reacting XIII-10 with chloromethyl pivalate in THF in the presence of Cs₂CO₃, then Suzuki Coupling with XIII-9 as described above in the synthesis of IT082. ¹H NMR (DMSO-d₆ 400 MHz): δ 9.78 (s, 1H), 7.68 (s, 1H), 7.46 (s, 1H), 7.27-7.36 (m, 6H), 5.79 (q, J=6.8 Hz, 1H), 5.70 (s, 2H), 3.62 (s, 3H), 1.66 (s, 2H), 1.52 (br, 5H), 1.12 (s, 9H). MS (ESI) m/z (M+H)⁺ 606.0.

IT088 was prepared by first reacting XIII-10 with 2-methoxyphenol in DCM in the presence fo DIEA and HATU to form the corresponding aryl ester, then Suzuki Coupling with XIII-9 as described above in the synthesis of IT082. ¹H NMR (400 MHz, Methanol-d₄): δ 7.60 (s, 1H), 7.30-7.34 (m, 2H), 7.23-7.25 (m, 3H), 7.07-7.09 (m, 3H), 7.01-7.02 (m, 2H), 6.92-6.97 (s, 1H), 5.83-5.88 (q, J=6.4 Hz, 1H), 3.85 (s, 3H), 3.69 (s, 3H), 1.95-1.98 (m, 2H), 1.58-1.63 (m, 5H). MS (ESI) m/z (M+H)⁺ 598.0.

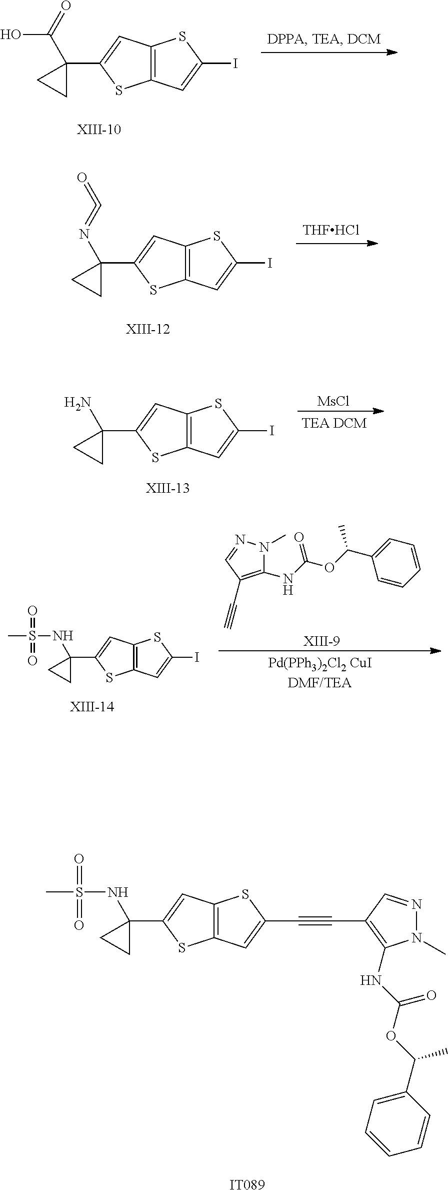

To a solution of XIII-10 (500 mg, 1.43 mmol) in DCM (12 mL) was added DPPA (470 mg, 1.7 mmol) and TEA (286 mg, 2.86 mmol). The reaction mixture was stirred at rt overnight. The mixture was diluted with DCM, washed with brine, and concentrated. The residue was purified by column (PE/EA=10/1) to give XIII-12 (400 mg, yield: 81%).

To a solution of XIII-12 (700 mg, 1.6 mmol) in THF (10 mL) was added 6N HCl (10 mL). The reaction mixture was heated to 70° C. and stirred for 6 h. The mixture was diluted with EtOAc, washed with brine, dried over Na₂SO₄, filtered and concentrated to give XIII-13 (600 mg, yield: 92%).

To a stirred mixture of XIII-13 (600 mg, 1.87 mmol), TEA (374 mg, 3.74 mmol) in DCM (10 mL) was added MsCl (234 mg, 2.06 mmol). The reaction mixture was flushed with nitrogen and stirred for 1 h at 25° C. The mixture was concentrated and diluted with EtOAc, washed with water and brine, dried over Na₂SO₄, filtered and concentrated. The mixture was concentrated and purified by column (PE/EA=5/1) to give XIII-14 (600 mg, yield: 80%).

IT089 was prepared by Suzuki Coupling of XIII-14 with XIII-9 as described above in the synthesis of IT082. ¹H NMR (400 MHz, DMSO-d₆): δ 9.79 (s, 1H), 8.43 (s, 1H), 7.70 (s, 1H), 7.48 (s, 1H), 7.33-7.38 (m, 4H), 7.26-7.30 (m, 2H), 5.77-5.82 (q, 1H), 3.63 (s, 3H), 2.74 (s, 3H), 1.52-1.54 (d, J=6.4 Hz, 3H), 1.42-1.43 (m, 2H), 1.26-1.29 (m, 2H). MS (ESI) m/z (M+H)⁺541.0.

IT090 was prepared by reacting XIII-10 with 2-chloro-N,N-dimethylacetamide in DMF in the presence of Cs₂CO₃, then Suzuki Coupling with XIII-9 as described above in the synthesis of IT082. ¹H NMR (CDCl₃, 400 MHz) δ 7.59 (s, 1H), 7.28-7.36 (m, 5H), 7.22 (s, 1H), 7.16 (s, 1H), 6.52 (br, 1H), 5.97 (q, J=6.4 Hz, 1H), 4.43 (s, 2H), 3.74 (s, 3H), 2.95 (s, 3H), 2.93 (s, 3H), 1.89-1.92 (m, 2H), 1.62 (s, 3H), 1.45-1.48 (m, 2H). MS (ESI) m/z (M+H)⁺ 577.0.

IT097 was prepared following the alternative synthesis of IT017 using (R)-1-phenylethyl (4-iodo-1-methyl-1H-pyrazol-5-yl)(methyl)carbamate in place of XIII-7. MS (ESI) m/z (M+H)⁺ 505.9. Sodium salt IT097a: ¹H NMR (DMSO-d₆, 400 MHz): δ 7.66 (s, 1H), 7.36 (s, 1H), 7.26-7.31 (m, 5H), 6.83 (s, 1H), 5.78-5.83 (q, J=6.4 Hz, 1H), 3.64 (s, 3H), 3.25 (s, 3H), 1.49 (d, J=6.4 Hz, 3H), 1.46-1.47 (m, 2H), 0.90-0.93 (m, 2H). MS (ESI) m/z (M+H)⁺ 506.0.

IT098 was prepared by reacting XIII-10 with methanesulfonamide in the presence of HATU and DIEA in DCM, followed by Suzuki coupling with XIII-9 using the same procedure described above. ¹H NMR (400 MHz, DMSO-d₆): δ 9.80 (brs, 1H), 7.60 (s, 1H), 7.28-7.41 (m, 7H), 5.77-5.78 (q, 1H), 3.61 (s, 3H), 2.99 (s, 3H), 1.51-1.52 (m, 5H), 1.23 (brs, 2H). MS (ESI) m/z (M+H)⁺ 568.9.

IT099 was prepared by two-step reduction reactions of IT017. First, a mixture of IT017 (0.2 g, 0.406 mol) and PtO₂ (20 mg) in MeOH (10 mL) was hydrogenated under 45 Psi of hydrogen pressure for 2 h at rt. The suspension was filtered through a pad of silica gel and the filter cake was washed with MeOH. The combined filter was concentrated to give an intermediate (160 mg, yield: 79.68%), which was mixed with and Pd/C (20 mg) in MeOH (10 mL) and hydrogenated under 45 Psi of hydrogen pressure for 2 h at rt. The suspension was filtered through a pad of silica gel and the filter cake was washed with MeOH. The organic layers was washed with brine, dried over Na₂SO₄, and concentrated. The residue was purified by prep-HPLC to afford IT099 (100 mg, yield: 62.66%). MS (ESI) m/z (M+H)⁺ 495.9. Sodium salt IT099a: ¹H NMR (DMSO-d₆, 400 MHz): δ 9.54 (s, 1H), 7.28-7.29 (m, 5H), 7.21 (s, 1H), 6.86 (s, 1H), 6.83 (s, 1H), 5.75 (d, J=6.4 Hz, 1H), 3.53 (s, 3H), 2.93 (t, J=7.6 Hz, 2H), 2.25 (t, J=7.6 Hz, 2H), 1.51 (br, 2H), 1.39 (br, 3H), 0.95 (br, 2H). MS

(ESI) m/z (M+H)⁺ 495.9.

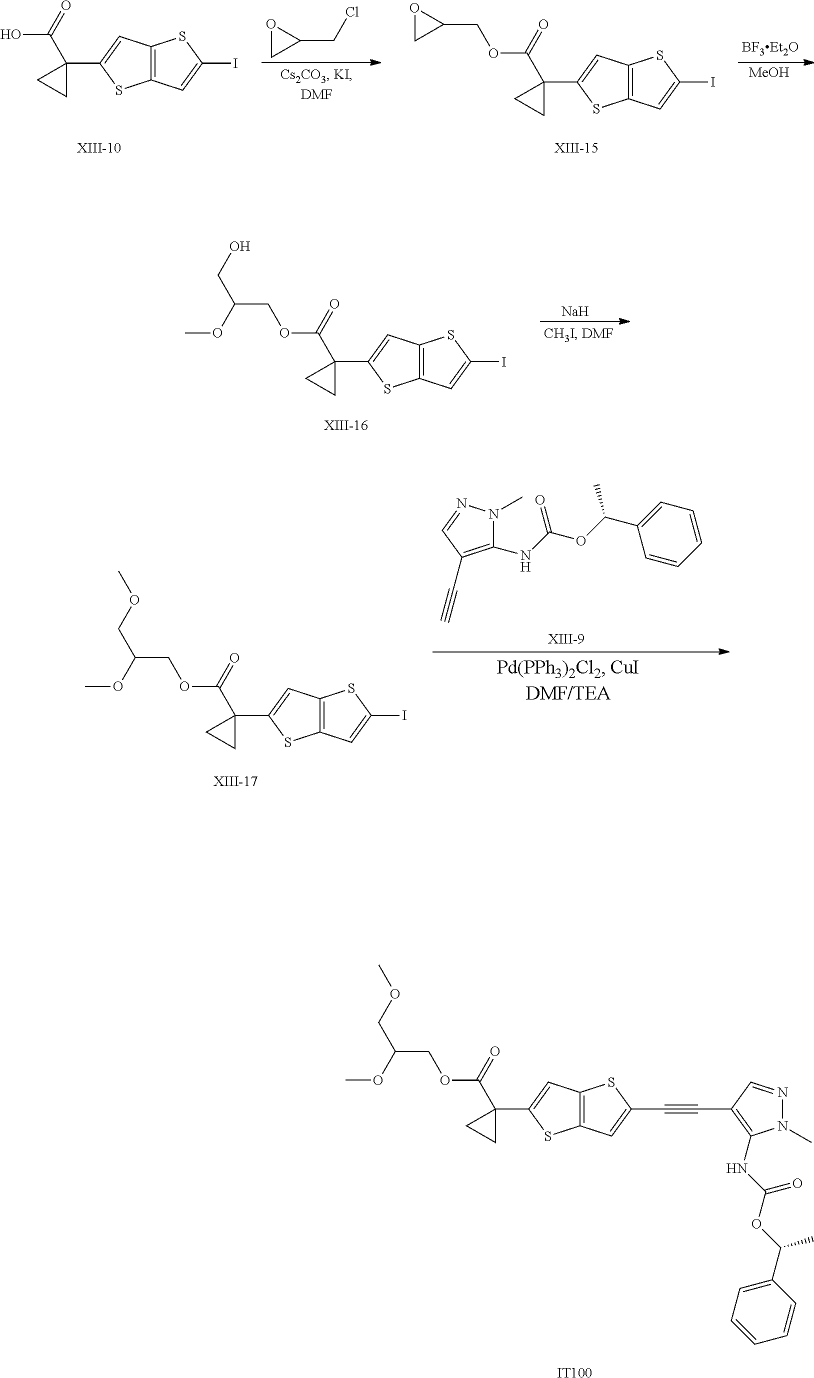

To a solution of XIII-10 (500 mg, 1.43 mmol) in DMF (10 mL) was added Cs₂CO₃ (930 mg, 2.86 mmol), KI (23 mg, 0.143 mmol), and 2-(chloromethyl)oxirane (160 mg, 1.74 mmol). The reaction mixture was heated at 70° C. for 12 h. The mixture was washed with water, extracted with EtOAc. The organics were combined, washed with saturated NaHCO₃, brine, dried with Na₂SO₄, filtered and concentrated to afford XIII-15 (165 mg, yield: 28.4%).

To a solution of XIII-15 (83 mg, 0.2 mmol) in MeOH (10 mL) was added BF₃.Et₂O (15 mg, 0.1 mmol) at −34° C. Then the reaction mixture was stirred at 4° C. for 12 h. The mixture was diluted with EtOAc, washed with H₂O. The organics were combined, washed brine, dried with Na₂SO₄, filtered and concentrated. The residue was purified by prep-TLC (PE/EA=3/1) to give XIII-16 (50 mg, yield: 55.8%).

To a solution of XIII-16 (25 mg, 0.057 mmol) and CH₃I (12 mg, 0.085 mmol) in DMF (2.5 mL) was added NaH (3 mg, 0.075 mmol, 60%) at −20° C. The mixture was stirred at 4° C. for 12 h. Then the mixture was quenched with H₂O, and extracted with EtOAc. The organics were combined, washed brine, dried with Na₂SO₄, filtered and concentrated. The residue was purified by prep-TLC (PE/EA=3/1) to give XIII-17 (8 mg, yield: 31%).

IT100 was obtained by Suzuki Coupling of XIII-17 and XIII-9 using the procedure described above. ¹HNMR (Methanol-d₄, 400 MHz) δ 7.61 (s, 1H), 7.22-7.41 (m, 7H), 5.84-5.89 (q, 1H), 4.28-4.30 (m, 1H), 4.07-4.12 (m, 1H), 3.70 (s, 3H), 3.51-3.53 (m, 1H), 3.39-3.40 (m, 2H), 3.38 (s, 3H), 3.30 (s, 3H), 1.73-1.76 (m, 2H), 1.60 (d, J=6.4 Hz, 3H), 1.47-1.49 (m, 2H). MS (ESI) m/z (M+H)⁺ 594.0.

IT101 was prepared following the similar procedure described in the alternative synthesis of IT017 using (R)-1-phenylethyl (5-ethynylthiazol-4-yl)carbamate in place of XIII-9. MS (ESI) m/z (M+H)⁺ 495.1. ¹H NMR (Methanol-d₄, 400 MHz): δ 8.83 (s, 1H), 7.40-7.42 (d, J=7.2 Hz, 2H), 7.30-7.34 (m, 2H), 7.25-7.27 (m, 2H), 7.19 (s, 1H), 5.87 (q, J=6.4 Hz, 1H), 1.74-1.76 (m, 2H), 1.59 (d, J=6.4 Hz, 3H), 1.44-1.46 (m, 2H).

Preparation of IT103: t-BuOOH (185 mg, 2.04 mmol) was added to a solution of IT017 (200 mg, 0.407 mmol), NaSO₂CF₃ (190 mg, 1.22 mmol) and CuSO₄ (6.4 mg, 0.04 mmol) in DMSO (10 mL). The reaction mixture was stirred at 30° C. for 24 h. Then additional t-BuOOH (185 mg, 2.04 mmol) and NaSO₂CF₃ (190 mg, 1.22 mmol) was added to the reaction mixture. The reaction mixture was stirred at 30° C. for additional 24 h. The reaction mixture was diluted with EtOAc and water. The aqueous layer was separated and extracted with EtOAc. Following standard work-up procedure, the filtrate was evaporated in vacuum and the residue was purified by prep-HPLC (containing 0.1% HCl) to afford IT103 (21 mg, yield 9.2%). ¹H NMR (DMSO-d₆, 400 MHz): δ 12.9 (br, 1H), 9.84 (br, 1H), 7.75 (s, 1H), 7.32-7.40 (m, 5H), 7.27-7.29 (m, 1H), 5.79 (t, J=6.0 Hz, 1H), 3.65 (s, 3H), 1.68-1.71 (m, 2H), 1.53 (d, J=6.0 Hz, 3H), 1.45-1.47 (m, 2H). MS (ESI) m/z (M+H)⁺ 559.9.

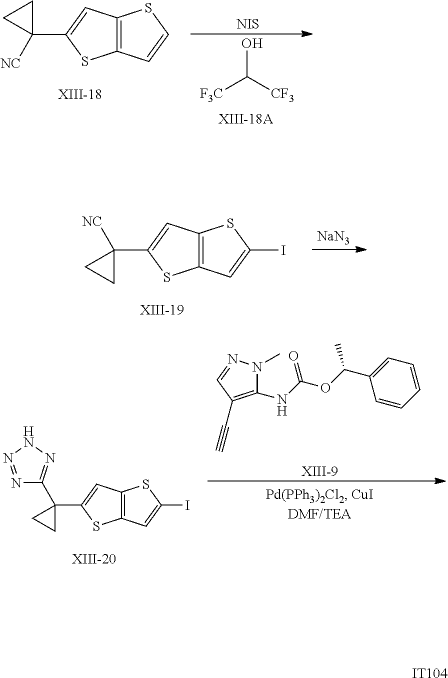

To a solution of XIII-18 (2.05 g, 10 mmol) in XIII-18A (10 mL) was added NIS (2.47 g, 11 mmol) at rt. The reaction mixture was stirred for overnight. The mixture was diluted with water and extracted with EtOAc. The combined organic layers were washed with brine, and concentrated under reduced pressure. The residue was purified to give XIII-19 (1.60 g, yield: 48.3%). To a solution of XIII-19 (660 mg, 2.0 mmol) and Et₃N.HCl (1110 mg, 8 mmol) in toluene (15 mL) was added NaN₃ (6.81 g, 56.75 mmol) at rt, then the mixture was heated to 100° C. for 14 h. After being diluted with EtOAc, the organic layer was washed with brine, dried over MgSO₄, and concentrated in vacuo to give a residue, which was purified by prep-HPLC to afford XIII-20 (200 mg, yield: 26.9%).

XIII-20 was subjected to Suzuki-coupling with XIII-9 following the same procedure in the alternative synthesis of XIII-6 to afford the final product IT104. ¹H NMR (DMSO-d₆, 400 MHz): δ : 7.71 (s, 1H), 7.50 (s, 1H), 7.47 (s, 1H), 7.29-7.36 (m, 5H), 5.79 (q, 1H), 3.63 (s, 3H), 1.74 (m, 2H), 1.72 (m, 2H), 1.54 (d, J=6.4 Hz, 3H). MS (ESI) m/z (M+H)⁺ 515.9.

### Example 6-C

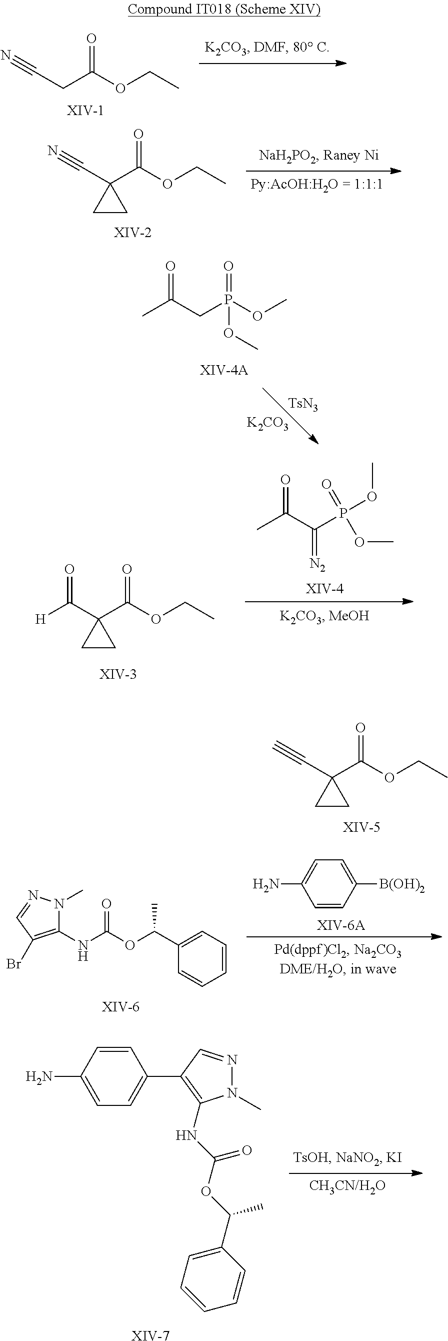

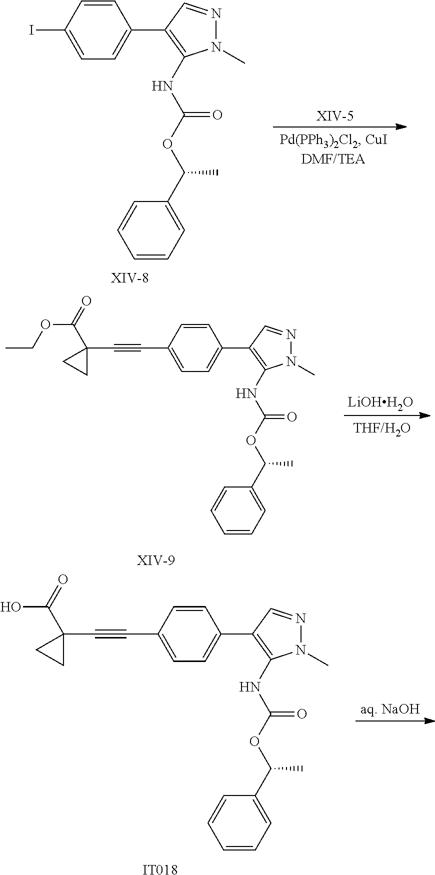

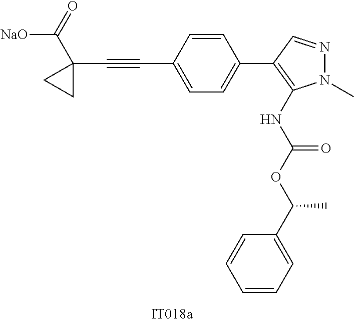

XIV-5 was prepared from ethyl 2-cyanoacetate in three steps reactions.

To a solution of XIV-6 (500 mg, 1.55 mmol) in DME/H₂O (v/v=3/1, 8 mL), Na₂CO₃ (821 mg, 7.75 mmol) and XIV-6A (592 mg, 2.32 mmol) were added, the resulting mixture was purged with nitrogen, then Pd (dppf)Cl₂ (113 mg, 0.16 mmol) was added. The reaction mixture was heated to 110° C. for 60 min. under nitrogen protection. After completion of the reaction, the mixture was poured into water, extract with EtOAc (10 mL×3). The combined organic layers were dried over Na₂SO₄, concentrated in vacuo. The residue was purified by chromatography (PE:EA=1:1) to afford XIV-7 (300 mg, yield: 57.7%).

To a solution of p-TsOH (700 mg, 3.57 mmol) in CH₃CN (1 mL) was added dropwise XIV-7 (400 mg, 1.19 mmol) in CH₃CN (2 mL), then the stirred mixture was cooled to 10-15° C. KI (492 mg, 2.98 mmol) and NaNO₂ (164 mg, 2.38 mmol) in H₂O (1.5 mL) was added to the reaction mixture. After addition, the mixture was stirred at rt for 3 hrs. After completion of the reaction, the mixture was poured into water, extract with EtOAc. The combined organic layers were washed with aq. Na₂SO₃, brine and dried over Na₂SO₄, concentrated in vacuo. The residue was purified by chromatography (PE:EA=2:1) to afford XIV-8 (350 mg, yield: 66%).

To a stirred mixture of XIV-8 (344 mg, 0.77 mmol), XVI-5 (crude) and CuI (49 mg, 0.26 mmol) in DMF (5 mL) and TEA (1 mL) was added Pd(PPh₃)₂Cl₂ (54 mg, 0.08 mmol). The reaction mixture was flushed with N₂ and stirred at rt overnight. The mixture was diluted with EA, washed with water and brine, dried over Na₂SO₄, filtered and concentrated. The residue was purified by chromatography (PE:EA=2:1) to afford XIV-9 (380 mg, crude yield: 100%).

IT018 and its sodium salt IT018a were prepared following the similar procedure described in the preparation of IT001 and IT001a. IT018: MS (ESI) m/z (M+H)⁺ 430.1. IT018a: ¹HNMR (Methanol-d₄, 400 MHz): δ 7.70 (s, 1H), 7.19-7.40 (m, 9H), 5.81 (br, 1H), 3.68 (s, 3H), 1.59 (br, 3H), 1.45 (d, J=3.6 Hz, 2H), 1.14 (d, J=3.2 Hz, 2H). MS (ESI) m/z (M+H)⁺ 430.1.

### Example 7

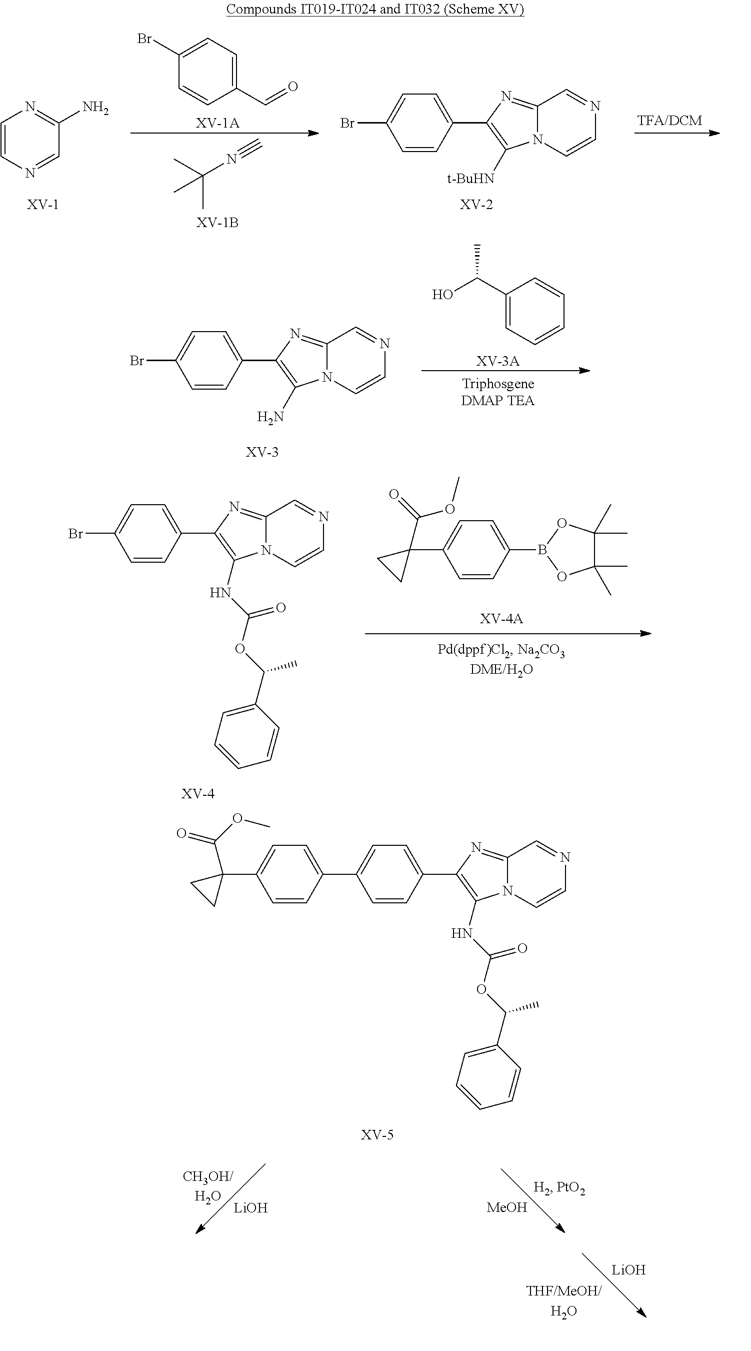

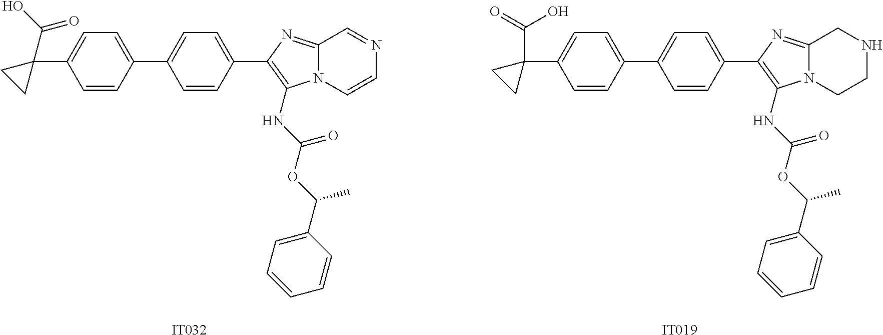 [SMILES: C[C@@H](OC(=O)Nc1c(-c2ccc(-c3ccc(C4(C(=O)O)CC4)cc3)cc2)nc2cnccn12)c1ccccc1 ; "IT032" ; C[C@@H](OC(=O)Nc1c(-c2ccc(-c3ccc(C4(C(=O)O)CC4)cc3)cc2)nc2n1CCNC2)c1ccccc1 ; "IT019"]

To XV-1 (1.08 g, 11.39 mmol) in 20 mL of MeOH were added XV-1A (3.39 g, 17 mmol), XV-1B (1.1 g, 13.1 mmol) and 1.0 N HCl O₄ in MeOH (1.14 mL, 1.14 mmol). The reaction mixture was stirred at rt for 8 h. Solvent was removed and the residue was purified by flash chromatography (PE:EA=1:1) to give XV-2 (2.5 g, yield: 64.1%).

A solution of XV-2 (2.5 g, 7.27 mmol) in 30 mL of DCM/TFA (v/v=4/1) was stirred at rt for 2 h. Solvent was removed, after neutralization with aqueous NaHCO₃, XV-3 (3.5 g, crude) was obtained and directly used in the next step.

To a solution of XV-3 (400 mg, 1.384 mmol) in 1,2-dichloroethane (10 mL) was added (R)-1-phenylethanol (422 mg, 6.92 mmol), TEA (699 mg, 6.92 mmol) and DMAP (168 mg, 0.692 mmol). The reaction mixture was stirred at rt for 6 h. The mixture was diluted with EtOAc, washed with brine, dried over Na₂SO₄, filtered and concentrated. The residue was purified by column chromatography (PE:EA=10:1) to give XV-4 (250 mg, yield: 41%).

XV-5 was prepared following the similar procedure as describe in the synthesis of III-5. MS (ESI) m/z (M+H)⁺ 532.2.

IT032 and its sodium salt IT032a were prepared following the similar procedure described in the synthesis of IT001 and IT001a. IT032: MS (ESI) m/z (M+H)⁺ 518.2. IT032a: ¹H NMR (DMSO-d₆ 400 MHz) δ 9.09 (s, 1H), 8.20 (s, 1H), 8.02 (d, J=7.6 Hz, 2H), 7.95 (d, J=4.4 Hz, 1H), 7.74 (d, J=6.4 Hz, 2H), 7.64 (d, J=8.0 Hz, 3H), 7.36-7.48 (m, 7H), 5.81 (q, 1H), 1.61 (br, 3H), 1.44 (br, 2H), 1.09 (br, 2H). MS (ESI) m/z (M+H)⁺ 518.2.

To a solution of XV-5 (200 mg, 0.376 mmol) in 4 mL of MeOH was added PtO₂ (20 mg). The reaction mixture was evacuated and back-filled with H₂ for 2 h at 40° C. LCMS showed that that the reaction was completed. The suspension was filtered through a pad of Celite and washed with MeOH (10 mL). The combined filtrates were concentrated and dissolved in MeOH:THF:H₂O=1:1:1 (12 mL). After hydrolysis with LiOH (78 mg, 1.86 mmol) overnight at rt, the solution was concentrated in vacuo, acidified, and extracted with EtOAc. The organic layer was isolated, concentrated, and purified to afford IT019 (120 mg, yield: 61.6%). MS (ESI) m/z (M+H)⁺523.2. Sodium salt IT019a: ¹H NMR (DMSO-d₆, 400 MHz): δ 7.74 (d, J=8.0 Hz, 2H), 7.28-7.49 (m, 11H), 5.73-5.81 (q, 1H), 3.80 (d, J=5.2 Hz, 2H), 3.55 (br, 2H), 2.98 (br, 2H), 2.66 (br, 1H), 1.48 (br, 3H), 1.17 (br, 2H), 0.67 (br, 2H). MS (ESI) m/z (M+H)⁺ 523.2.

IT020 was prepared following the similar procedure for the preparation of IT019 by reacting XV-5 with acetyl chloride in DCM and TEA, followed by LiOH hydrolysis. MS (ESI) m/z (M+H)⁺ 565.2. Sodium salt IT020a: ¹H NMR (DMSO-d₆, 400 MHz): δ :9.47 (s, 1H), 7.71 (s, 2H), 7.55 (m, 2H), 7.43-7.48 (m, 6H), 7.29-7.32 (m, 2H), 5.77 (q, 1H), 4.74 (br, 1H), 4.64 (br, 1H), 3.78-3.87 (m, 2H), 3.28 (s, 2H), 2.11 (s, 3H), 1.54-1.55 (m, 2H), 1.20 (br, 2H), 0.72 (br, 2H). MS (ESI) m/z (M+H)⁺ 565.2.

IT021 was prepared following the similar procedure for the preparation of IT019 by reacting XV-5 with MsCl in DCM and TEA, followed by LiOH hydrolysis. MS (ESI) m/z (M+H)⁺ 601.2. Sodium salt IT021a: ¹H NMR (DMSO-d₆, 400 MHz): δ 9.61 (s, 1H), 7.36-7.75 (m, 13H), 5.79 (br, 1H), 4.46 (br, 2H), 3.82 (br, 4H), 3.07 (s, 3H), 1.58 (br, 3H), 1.35 (br, 2H), 0.96 (br, 2H). MS (ESI) m/z (M+H)⁺ 601.2.

IT022 was prepared following the similar procedure for the preparation of IT019 by reacting XV-5 with methylcarbamic chloride in DCM and TEA, followed by LiOH hydrolysis. MS (ESI) m/z (M+H)⁺ 594.2. Sodium salt IT022a: ¹H NMR (DMSO-d₆, 400 MHz): δ 9.45 (s, 1H), 7.73-7.75 (m, 2H), 7.37-7.59 (m, 11H), 6.82 (d, J=4.0 Hz, 1H), 5.78-5.79 (m, 1H), 4.55 (s, 2H), 4.74 (s, 2H), 3.67-3.77 (m, 4H), 2.61-2.65 (d, J=4.0 Hz, 3H), 1.56-1.57 (d, J=6.4 Hz, 3H), 1.38 (br, 2H), 1.01 (br, 2H). MS (ESI) m/z (M+H)⁺ 594.2.

IT023 was prepared following the similar procedure for the preparation of IT019 by reacting XV-5 with ethyl carbonochloridate in DCM and TEA, followed by LiOH hydrolysis. MS (ESI) m/z (M+H)⁺ 595.2. Sodium salt IT023a: ¹H NMR (DMSO-d₆, 400 MHz): δ 9.53 (s, 1H), 7.73 (d, J=6.8 Hz, 2H), 7.33-7.58 (m, 11H), 5.78-5.79 (m, 1H), 4.62 (br, 2H), 4.10-4.15 (q, J=7.2 Hz, 1H), 3.82 (br, 2H), 3.72 (br, 2H), 1.56-1.57 (d, J=5.2 Hz, 3H), 1.21-1.25 (m, 5H), 0.77 (br, 2H). MS (ESI) m/z (M+H)⁺ 595.2.

IT024 was prepared following the similar procedure for the preparation of IT019 by reacting XV-5 with ethyl iodide in DMF and TEA, followed by LiOH hydrolysis. MS (ESI) m/z (M+H)⁺ 551.2. Sodium salt IT024a: ¹H NMR (DMSO-d₆, 400 MHz): δ 7.59-7.66 (m, 8H), 7.39-7.47 (m, 5H), 5.84-5.85 (m, 1H), 4.21 (s, 2H), 4.05 (br, 2H), 3.40 (br, 2H), 3.06 (br, 2H), 1.61-1.62 (m, 5H), 1.32 (d, J=6.8 Hz, 3H), 1.23-1.25 (m, 2H). MS (ESI) m/z (M+H)⁺ 551.2.

### Example 8-A

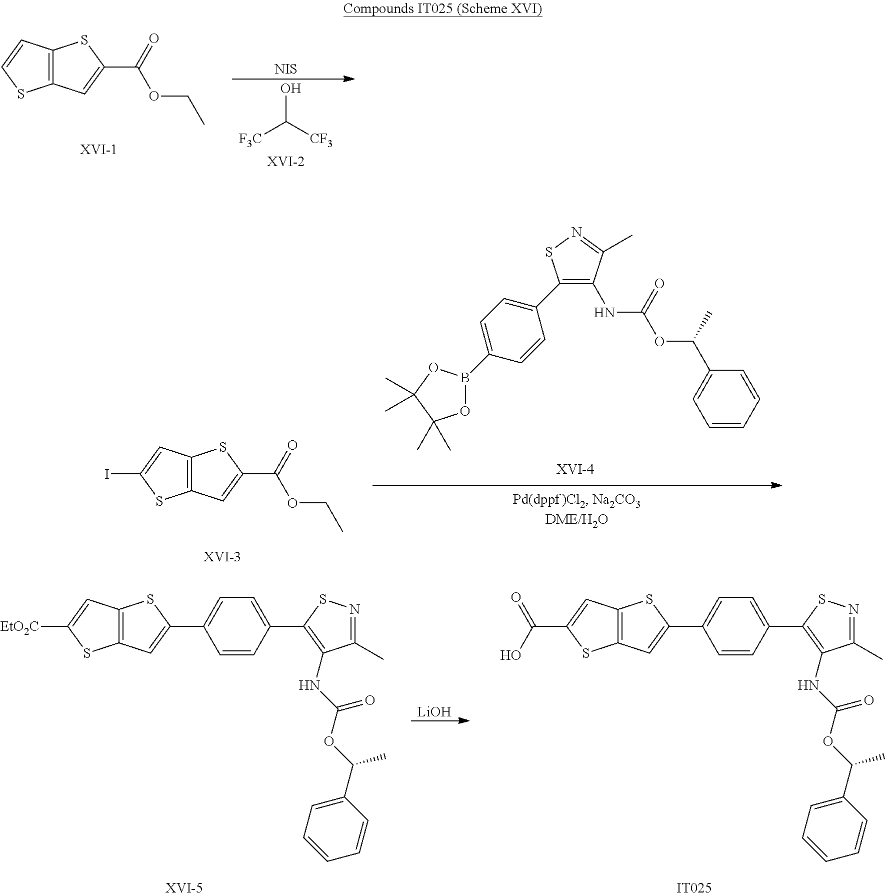

XVI-1 was prepared by reacting 3-bromothiophene-2-carbaldehyde with ethyl 2-mercaptoacetate and K₂CO₃ in DMF at 60° C. overnight under N₂ protection.

To a solution of XVI-1 (2.12 g, 10 mmol) in XVI-2 (10 mL) was added NIS (2.36 g, 10.5 mmol) at rt. The reaction mixture was stirred for overnight, and then the mixture was diluted with water and extracted with EtOAc. The combined organic layers were washed, concentrated under reduced pressure, and purified by column chromatography on silica gel (PE:EA=10:1) to give XVI-3 (2.14 g, yield: 63.3%). ¹H NMR (CDCl₃, 400 MHz): δ 7.88 (s, 1H), 7.45 (s, 1H), 4.38 (q, J=7.2 Hz, 2H), 1.40 (t, J=7.2 Hz, 3H).

XVI-5 was prepared by reacting XVI-3 and XVI-4 following the similar procedure as describe in the synthesis of III-5. MS (ESI) m/z (M+H)⁺ 532.2.

IT025 and its sodium salt IT025a were prepared following the similar procedure described in the synthesis of IT001 and IT001a. IT025a: ¹HNMR (DMSO-d₆, 400 MHz): δ 9.31 (s, 1H), 7.94 (s, 1H), 7.68-7.74 (m, 3H), 7.53-7.54 (m, 2H), 7.33-7.39 (m, 5H), 5.73 (br, 1H), 2.19 (s, 3H), 1.53 (br, 3H). MS (ESI) m/z (M+H)⁺ 521.0.

IT111 was prepared following the similar procedure for the synthesis of IT025 using methyl 1-(2-iodothieno[2,3-d]thiazol-5-yl)cyclopropanecarboxylate in place of XVI-3 and the isothiazole analog in place of XVI-4. MS (ESI) m/z (M+H)⁺ 546.1. ¹H NMR (DMSO, 400 MHz): δ 12.85 (s, 1H), 9.40 (s, 1H), 8.08 (d, J=7.2 Hz, 2H), 7.85 (d, J=7.2 Hz, 2H), 7.20-7.44 (m, 6H), 5.76 (s, 1H), 2.15 (s, 3H), 1.68 (s, 2H), 1.57 (d, J=5.2 Hz, 2H), 1.46 (s, 2H).

### Example 8-B

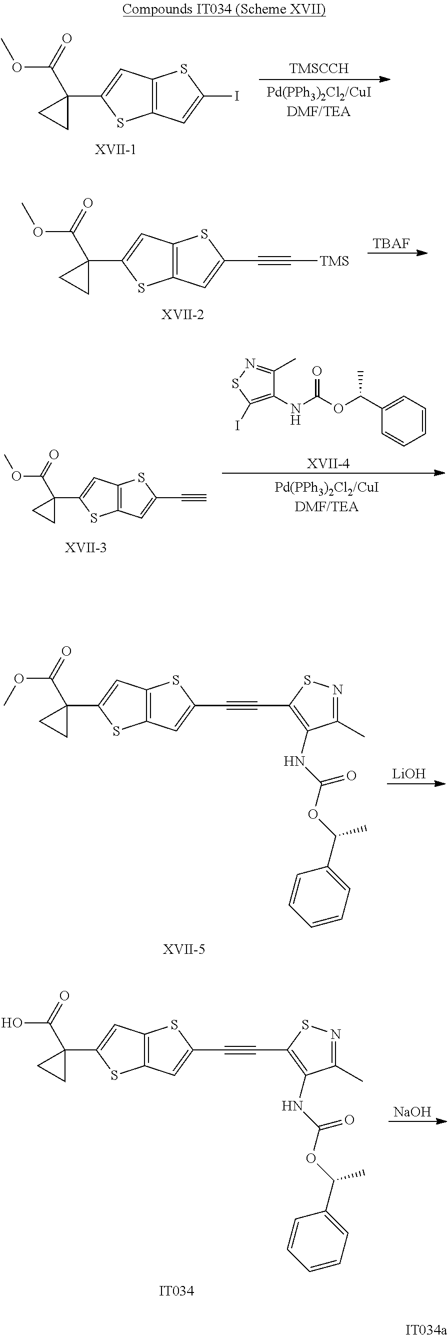

XVII-1 was prepared from XVI-1 by hydrolyzing the ethyl ester into hydroxy with LiAlH₄, converting the hydroxy group into nitrile, cyclization with 1-bromo-2-chloroethane, converting nitrile into methyl ester, and adding the iodo substituent with NIS in five steps.

To a mixture of XVII-1 (1.0 g, 2.75 mmol), CuI (27.7 mg, 0.14 mmol) and Pd(dppf)Cl₂ (96 mg, 0.14 mmol) in DMF/TEA (25 mL, v/v=3:1) was added TMSCCH (0.81 g, 8.24 mmol). The reaction mixture was stirred for 2 h and diluted with water, extracted with EtOAc, and the combined organic layer was washed with brine, dried over Na₂SO₄, concentrated to afford crude XVII-2 (810 mg, crude yield: 88.8%).

To a mixture of compound XVII-2 (810 mg, 242 mmol) in DCM (30 mL) was added TBAF (1.45 g, 6.05 mmol). The reaction mixture was stirred for 2 h and diluted with water, extracted with DCM, and the combined organic layer was washed with brine, dried over Na₂SO₄, concentrated. The residue was purified by chromatography on silica gel (PE:EA=10:1) to afford XVII-3 (390 mg, yield: 61.0%).

XVII-5 was prepared by reacting XVII-3 and XVII-4 following the similar procedure described in the preparation of 1-6. MS (ESI) m/z (M+H)⁺ 523.1.

IT034 and its sodium salt IT034a were prepared following the similar procedure described in the synthesis of IT001 and IT001a. IT034: MS (ESI) m/z (M+H)⁺ 509.0. IT034a: ¹H NMR (DMSO-d₆, 400 MHz): δ 7.49 (s, 1H), 7.30-7.40 (m, 5H), 6.87 (s, 1H), 5.79-5.81 (q, 1H), 2.28 (s, 3H), 1.55 (d, J=6.4 Hz, 3H), 1.48-1.49 (m, 2H), 0.99-1.00 (m, 2H). MS (ESI) m/z (M+H)⁺ 509.0.

IT074 was prepared following the general synthetic scheme of IT034 replacing XVII-4 with the corresponding carbamate (R)-1-phenylethyl (2-iodobenzofuran-3-yl)carbamate. MS (ESI) m/z (M+H)⁺ 528.0. Sodium salt IT074a: MS (ESI) m/z (M+H)⁺ 528.0. ¹H NMR (DMSO-d₆, 400 MHz): δ 9.83 (br s, 1H), 7.52-7.58 (m, 3H), 7.27-7.42 (m, 7H), 6.93 (s, 1H), 5.79-5.84 (q, 1H), 1.55 (d, J=6.4 Hz, 3H), 1.48 (br, 2H), 1.05 (br, 2H).

### Example 9

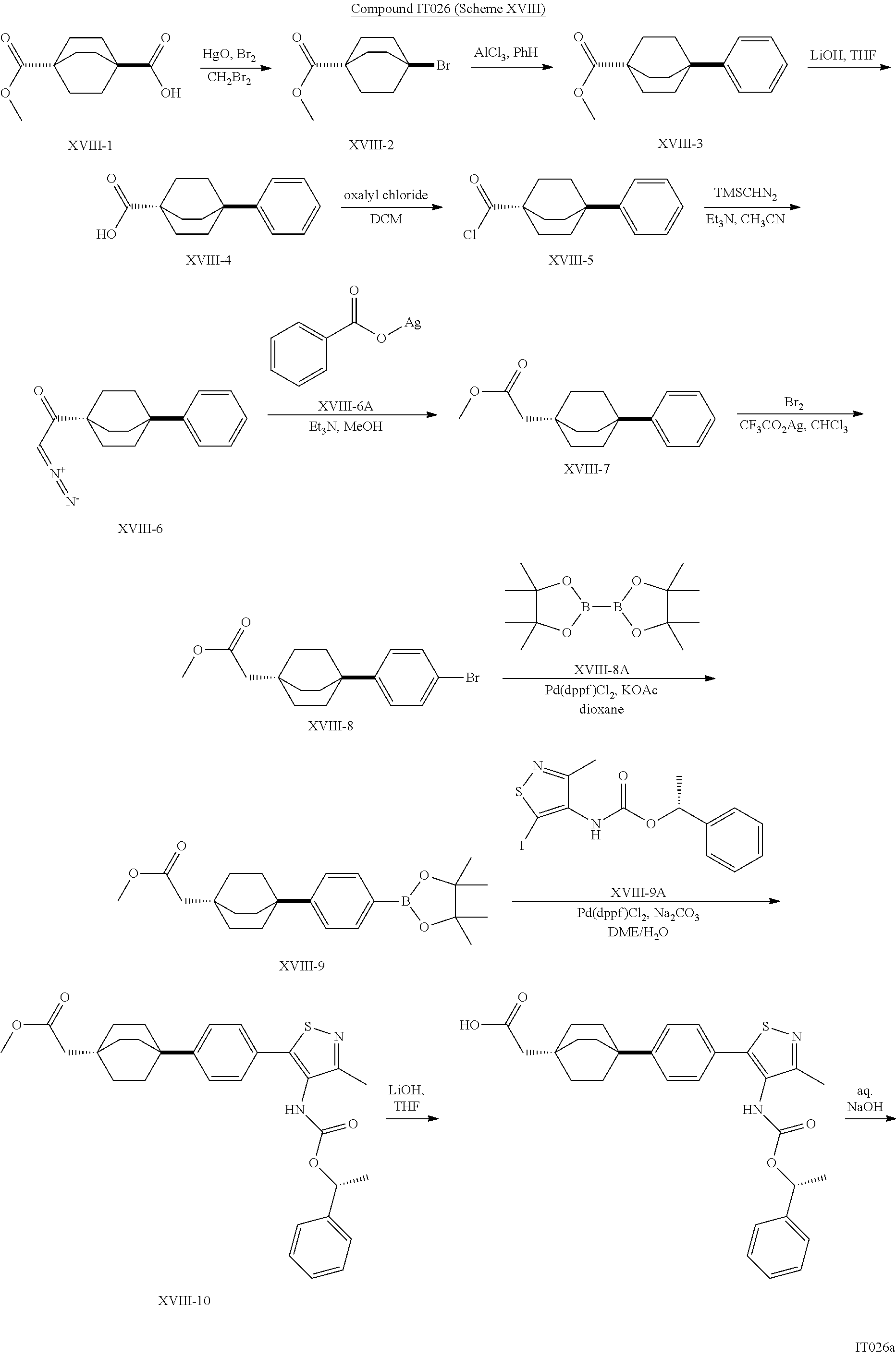

To a stirred mixture of XVIII-1 (10 g, 47.2 mmol) in CH₂Br₂ (100 mL) was added HgO (17.5 g, 80.3 mmol) at rt. The mixture was heated to 80° C. and Br₂ (3.6 mL, 47.2 mmol) was added dropwise during 40 min. After addition, the mixture was stirred at 80° C. for 3 h. Then the mixture was cooled to rt, and filtered. The filtrate was treated with MgSO₄, filtered and concentrated in vacuo. The residue XVIII-2 (11 g, yield 94.8%) was used in next step directly.

A solution of XVIII-2 (6 g, 24.2 mmol) in anhydrous benzene (60.15 g) was added dropwise to an ice-water cooled suspension of AlCl₃ (5.95 g, 45.1 mmol) in benzene (60.15 g) under nitrogen. The resulting reaction mixture was allowed to stirred in the ice bath for 30 min and then at rt overnight. The mixture was heated to 60° C. for 4 h and then allowed to cool to rt and poured into ice and concentrated HCl. The mixture was extracted with EtOAc, washed with brine, separated, and dried over Na₂SO₄ to leave an orange-brown solid, which was purified by column chromatography (PE:EA=10:1) to afford XVIII-3 (2.3 g, yield: 38.6%).

To a solution of XVIII-4 (1.5 g, 6.52 mmol) in DCM (25 mL) was added DMF (2 drops) followed by oxalyl chloride (1.23 g, 9.78 mmol). The reaction mixture was allowed to stir at rt overnight. The solvent was evaporated under reduced pressure to leave crude XVIII-5 (1.5 g, yield: 92.6%), which was used directly in the next step.

XVIII-5 (1.5 g, 6.05 mmol) was dissolved in a solution of MeCN/THF (v/v=1/1, 10 mL) and added dropwise to an ice water cooled solution of TMSCHN₂ (4.84 mL, 9.68 mmol) and TEA (1.22 g, 12.1 mmol) in a mixture of MeCN and THF (v/v=1/1, 15 mL). The reaction mixture was allowed to stir at 0° C. for 1 h and then for 5 h at rt. The solvent was removed under vacuum and the mixture was diluted with EtOAc and water, and the organic layer was separated, dried and concentrated. The residue was purified by column chromatography (PE:EA=10:1) to afford XVIII-6 (0.4 g, yield: 29.2%). MS (ESI) m/z (M+H)⁺ 255.2.

XVIII-6 (0.6 g, 2.36 mmol) in methanol (20 mL) and placed in an ultrasound bath, a solution of XVIII-6A (108 mg, 0.47 mmol) in TEA (953 mg, 9.44 mmol) was added dropwise, and the mixture was sonicated for 5 h at rt. The mixture was diluted with EtOAc, washed with brine, dried over Na₂SO₄, filtered and concentrated. The residue was purified by column chromatography (PE:EA=10:1) to afford XVIII-7 (350 mg, yield: 57%).

A chloroform (10 mL) solution of bromine (186 mg, 1.16 mmol) was added dropwise to a vigorously stirred mixture of XVIII-7 (300 mg, 1.16 mmol) and CF₃CO₂Ag (308 mg, 1.39 mmol) in chloroform (10 mL). After stiffing for 3 h, the mixture was diluted with EtOAc, washed with brine, dried over Na₂SO₄, filtered and concentrated. The residue was purified by column chromatography (PE:EA=10:1) to afford XVIII-8 (220 mg, yield: 56%).

XVIII-9 and XVIII-10 were prepared following the similar procedures described in the synthesis of III-3 and III-5.

IT026 and its sodium salt IT026a were prepared following the similar procedure described in the synthesis of IT001 and IT001a. IT026: MS (ESI) m/z (M+H)⁺ 505.1. IT026a: ¹HNMR (DMSO-d₆ 400 MHz) (57.38-7.51 (m, 9H), 5.72-5.73 (m, 1H), 2.23 (s, 3H), 1.78 (s, 2H), 1.71-1.72 (m, 6H), 1.52-1.65 (m, 6H), 1.51-1.52 (m, 3H). MS (ESI) m/z (M+H)⁺ 505.2.

IT093 was prepared following the similar synthetic scheme of IT026 using methyl 1-(4-phenylbicyclo[2.2.2]octan-1-yl)cyclopropanecarboxylate in place of XVIII-7. ¹H NMR (CDCl₃ 400 MHz): δ 7.31-7.38 (m, 9H), 6.02 (s, 1H), 5.85 (br, 1H), 2.37 (s, 2H), 1.81-1.83 (m, 6H), 1.71-1.83 (m, 6H), 1.57 (br, 3H), 0.97 (br, 2H), 0.79 (br, 2H). MS (ESI) m/z (M+H)⁺ 531.2.

### Examnle 10

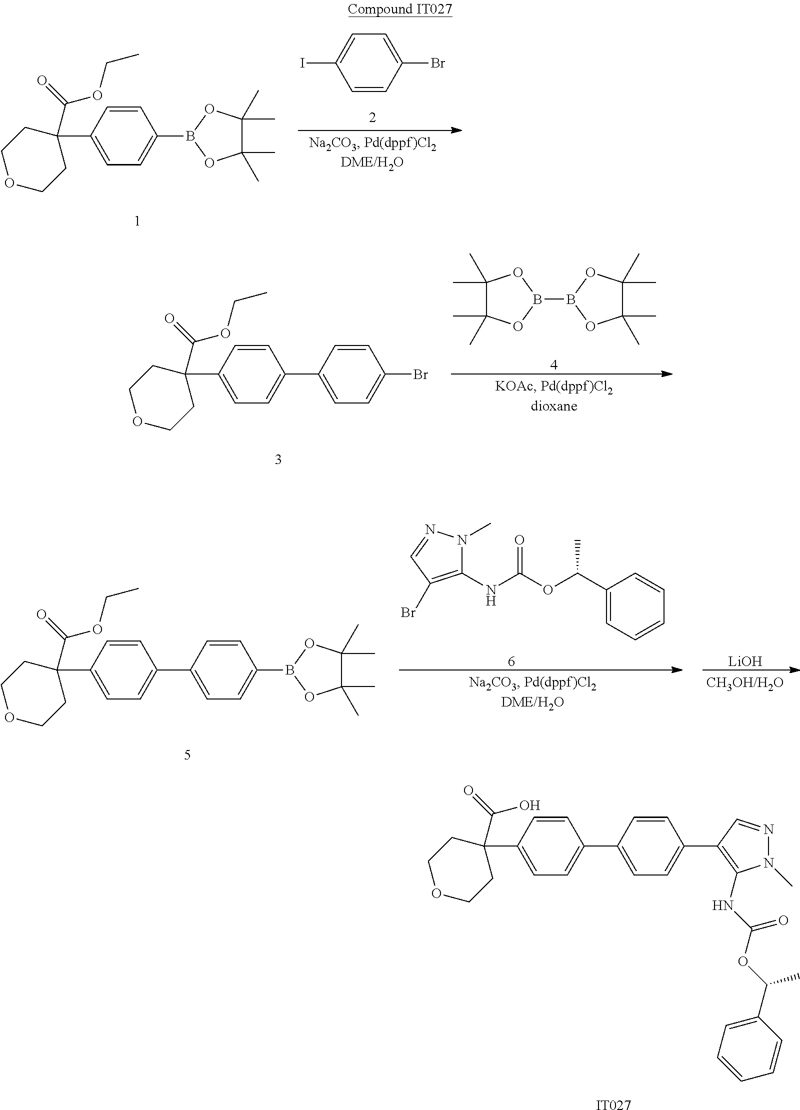

IT027 was prepared from compound 1 as described in the scheme above and followed the similar procedures as described in the synthesis of III-3, III-5 and IT004. Sodium salt IT027a: ¹H NMR (Methanol-d₄, 400 MHz) δ 7.36-7.73 (m, 14H), 5.8 (m, 1H), 3.88 (d, J=11.8 Hz, 2H), 3.70-3.77 (m, 5H), 2.53 (d, J=13.2 Hz, 2H), 1.89 (t, J=10.0 Hz, 2H), 1.60 (s, 3H). MS (ESI) m/z (M+Na)⁺526.2.

IT042 was prepared following the similar synthetic scheme as IT027, using 1-bromo-2,5-difluoro-4-iodobenzene in place of compound 2 and VI-6A in place of compound 6. Sodium salt IT042a: ¹H NMR (400 MHz, DMSO-d₆): δ 7.47-7.57 (m, 7H), 7.26-7.45 (m, 4H), 5.69 (br, 1H), 3.71-3.73 (m, 2H), 3.51-3.56 (m, 2H), 2.43-2.46 (m, 2H), 2.29 (s, 3H), 1.48-1.59 (m, 5H). MS (ESI) m/z (M+H)⁺ 579.1.

IT044 was prepared following the similar synthetic scheme as IT027, using 1-bromo-2,5-difluoro-4-iodobenzene in place of compound 2. IT044: MS (ESI) m/z (M+H)⁺ 562.2. Sodium salt IT044a: ¹H NMR (DMSO-d₆, 400 MHz): (59.76 (s, 1H), 7.73 (s, 1H), 7.51-7.74 (m, 11H), 5.76 (br, 1H), 3.52-3.68 (m, 7H), 2.45-2.46 (m, 2H), 1.67-1.78 (m, 2H), 1.50 (br, 3H). MS (ESI) m/z (M+H)⁺ 562.2.

IT045 was prepared following the similar procedure for the synthesis of IT042. IT045: MS (ESI) m/z (M+H)⁺ 565.1. IT045a: ¹H NMR (400 MHz, Methanol-d₄): δ 7.61 (d, J=8.0 Hz, 2H), 7.53 (d, J=8.0 Hz, 2H), 7.32-7.40 (m, 7H), 5.16 (s, 2H), 3.87-3.90 (m, 2H), 3.72-3.77 (m, 2H), 2.53-2.57 (m, 2H), 2.38 (s, 3H), 1.86-1.92 (m, 2H). MS (ESI) m/z (M+H)⁺ 565.1.

### Example 11

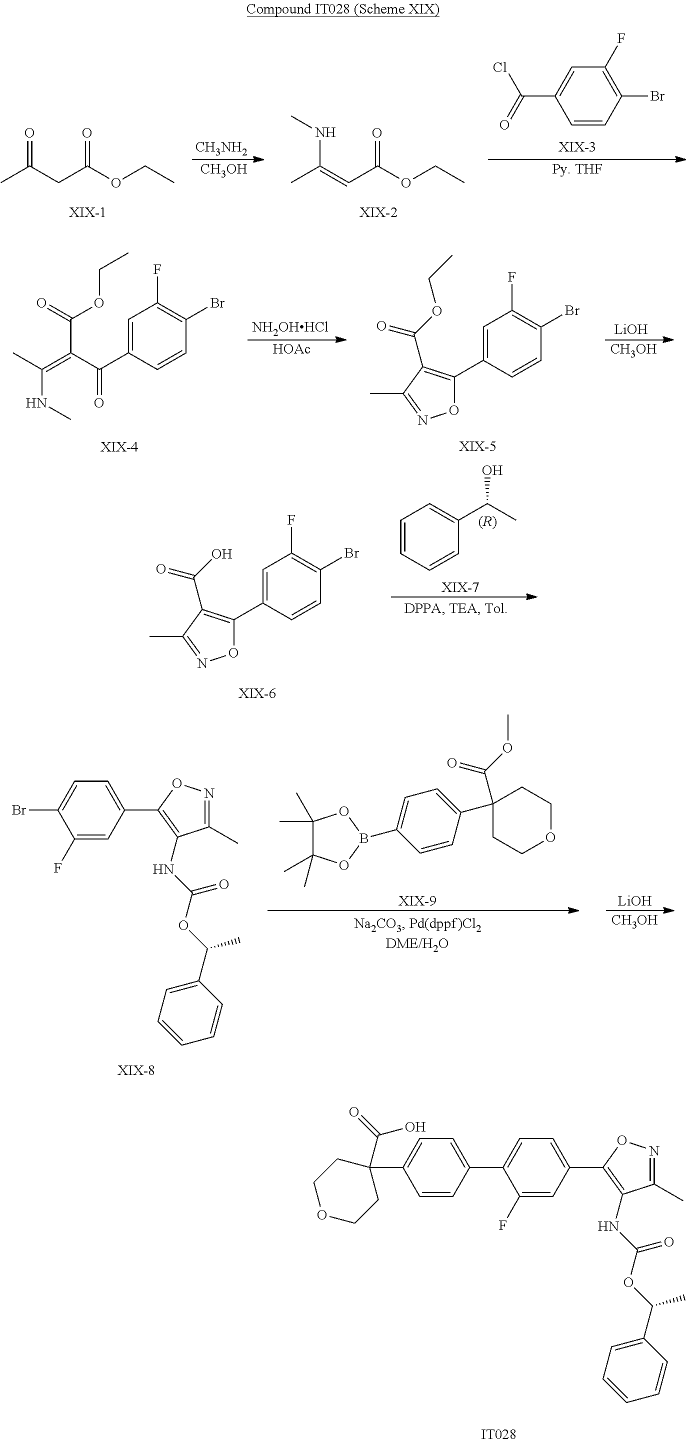

Methylamine solution in MeOH (90.3 g, 768 mmol, 27% w/w) was added into XIX-1 (50 g, 384 mmol) at rt, then the mixture was heated to 45° C. for 18 h. After being cooled to rt., the mixture was extracted with DCM, and the combined organic layer was washed with water, dried over Na₂SO₄, and concentrated in vacuum to give XIX-2 (49 g, yield 89%) without purification.

To a stirred solution of XIX-2 (2.15 g, 13.7 mmol) and pyridine (1.08 g, 13.7 mmol) in THF was added dropwise XIX-3 (3.17 g, 13.7 mmol) at 0° C. under nitrogen. The solution was stirred for 0.5 h, then warmed slowly to rt and stirred overnight. H₂O (20 mL) was added, and the mixture was extracted with EtOAc. The organic layer was combined and washed with brine, dried over Na₂SO₄, concentrated in vacuum to afford XIX-4 (4.5 g, crude yield 95.7%) as a yellow solid, and used in nest step directly.

To a stirred solution of crude XIX-4 (4.5 g, 13.7 mmol) in HOAc (30 mL) was added hydroxylamine hydrochloride (0.95 g, 13.7 mmol) under nitrogen. After the addition, the solution was heated to reflux under nitrogen for 2 h. The solvent was removed under vacuum and the residue was purified by column chromatography on silica gel (PE:EA=5:1) to afford XIX-5 (2.5 g, yield 58%) as a white solid, followed by LiOH hydrolysis in MeOH/H₂O (v/v=5:1) refluxing under nitrogen for 1 h. MeOH was removed in vacuo and the residue was adjusted to pH=2. After standard work-up procedure and purification, XIX-6 (2.0 g, yield 85%) was obtained as a white solid.

The mixture of XIX-6 (1 g, 3.3 mmol), XIX-7 (0.49 g, 4 mmol), DPPA (1.1 g, 4.0 mmol) and Et₃N (0.7 g, 2.6 mmol) in toluene (30 mL) was heated to reflux under nitrogen for 1 h. The mixture was concentrated, and the residue was partitioned between H₂O and DCM. The organic layer was washed with brine, dried over Na₂SO₄, and concentrated. The residue was purified by column chromatography on silica gel (PE:EA=3:1) to afford XIX-8 (0.9 g, yield 65%) as a white solid.

IT028 and its sodium salt IT028a were prepared following the similar procedures for the preparation of III-5, IT001 and IT001a. IT028: MS (ESI) m/z (M+Na)⁺545.2. IT028a: ¹H NMR (Methanol-d₄, 400 MHz): δ 7.29-7.61 (m, 12H), 5.80-5.82 (q, 1H), 3.87-3.90 (m, 2H), 3.71-3.77 (m, 2H), 2.54 (d, J=12.4 Hz, 2H), 2.17 (s, 3H), 1.85-1.93 (m, 2H), 1.56 (d, J=5.6 Hz, 3H). MS (ESI) m/z (M+Na)⁺545.2.

IT029 was prepared following the similar procedure for the synthesis of IT028 using 4-chloro-2,5-difluorobenzoyl chloride to replace XIX-3 to afford a yellow solid. Sodium salt IT029a: MS (ESI) m/z (M+H)⁺ 563.2. ¹H NMR (Methanol-d₄, 400 MHz): δ 7.31-7.64 (m, 11H), 5.76-5.78 (q, 1H), 3.89-3.92 (m, 2H), 3.74-3.79 (m, 2H), 2.57 (d, J=12.8 Hz, 2H), 2.25 (s, 3H), 1.88-1.93 (m, 2H), 1.56 (d, J=12.8 Hz, 3H).

### Example 12

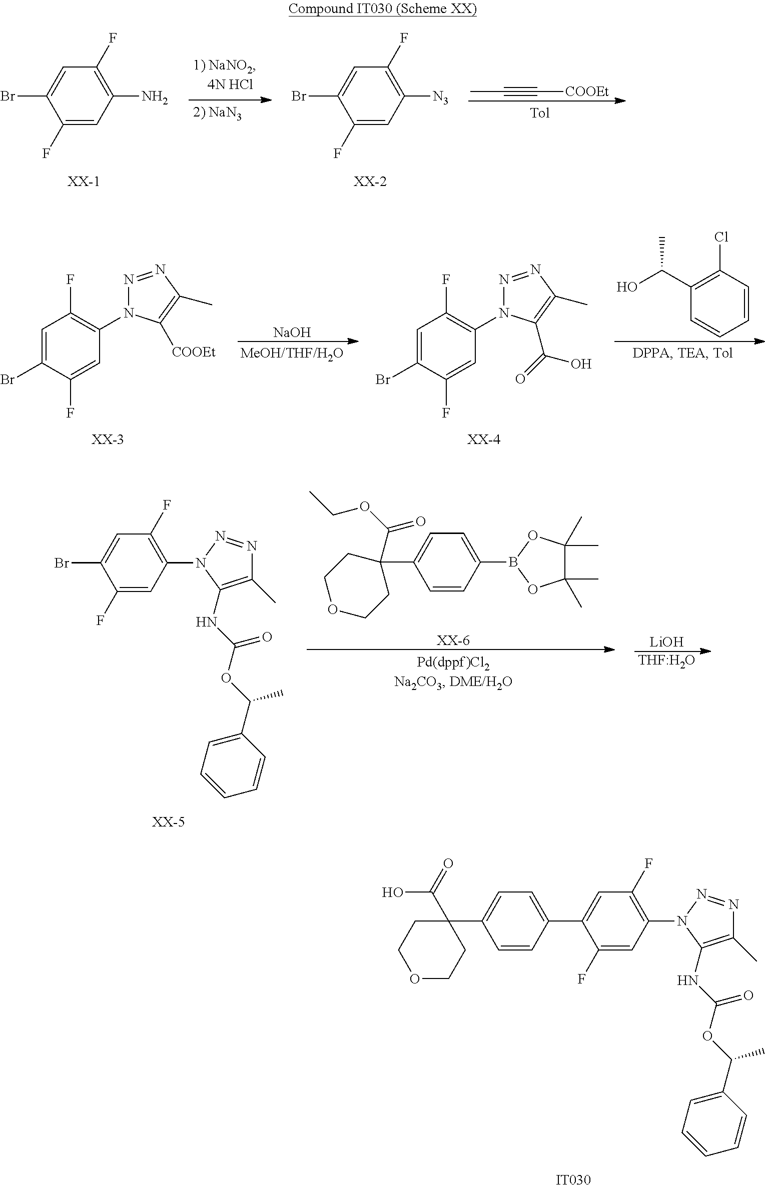

To a solution of XX-1 (5 g, 24 mmol) in 4N hydrochloride solution (36 mL) was added dropwise of NaNO₂ (1.84 g, 26.7 mmol) in water (10 mL) at 0° C. After addition, the mixture was stirred for 30 minutes, then NaN₃ (1.89 g, 29.3 mmol) was added. The reaction mixture was slowly warmed to rt and stirred for 1 h. The reaction mixture was extracted with MTBE. The combined organic phase was dried over Na₂SO₄, filtered and concentrated to give crude XX-2 (5.63 g, crude yield: 100%), which was used to next step directly.

To a solution of XX-2 (5.63 g, 24.27 mmol) in toluene (50 mL) was added But-2-ynoic acid ethyl ester (3.36 mL, 29.1 mmol). The reaction mixture was flushed with nitrogen and heated to reflux overnight. The reaction mixture was concentrated, and the residue was purified by column chromatography (PE:EA=5:1) to give XX-3 (6 g, yield: 71.5%).

To a solution of XX-3 (1 g, 2.89 mmol) in MeOH/THF/H₂O (10 mL/10 mL/10 mL) was added NaOH (578 mg, 14.45 mmol). The reaction mixture was stirred at rt overnight. The mixture was cooled down to 0° C. and neutralized to pH=4.0 with 3N HCl. The mixture was extracted with EtOAc, dried over Na₂SO₄ and concentrated to give crude XX-4 (659 mg, yield: 71.7%), which was used to next step directly.

To a solution of XX-4 (459 mg, 1.627 mmol) in dry toluene (5 mL) was added (R)-1-phenylethanol (535 mg, 1.95 mmol), TEA (238 mg, 3.25 mmol) and DPPA (451 mg, 1.95 mmol). The reaction mixture was heated to 80° C. for 3 h. The mixture was diluted with EtOAc, washed with brine, dried over Na₂SO₄, filtered and concentrated. The residue was purified by column chromatography (PE:EA=10:1) to give XX-5 (490 mg, yield: 97%). MS (ESI) m/z (M+H)⁺438.0.

IT030 and its sodium salt IT030a were prepared following the similar procedures for the preparation of III-5, IT001 and IT001a. IT030: MS (ESI) m/z (M+H)⁺ 563.2. IT030a: ¹H NMR (DMSO-d₆, 400 MHz): δ 7.65 (d, J=8.4 Hz, 2H), 7.37-7.54 (m, 3H), 7.32-7.36 (m, 1H), 7.34-7.19 (m, 5H), 5.72-5.67 (q, 1H), 3.81-3.91 (m, 2H), 3.73-3.77 (m, 2H), 2.56 (d, J=12.4 Hz 2H), 2.26 (s, 3H), 1.87-1.93 (m, 2H), 1.47 (br, 3H). MS (ESI) m/z (M+H)⁺ 563.2.

IT072 was prepared following the general synthetic scheme for the synthesis of IT030, using 4-bromoaniline in place of XX-1, F₃C≡COOEt in place of But-2-ynoic acid ethyl ester, and

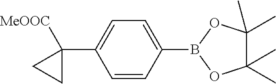 [SMILES: COC(=O)C1(c2ccc(B3OC(C)(C)C(C)(C)O3)cc2)CC1]

in place of XX-6. MS (ESI) m/z (M+H)⁺ 537.2. ¹H NMR (Methanol-d₄, 400 MHz): δ 7.75 (d, J=8.0 Hz, 2H), 7.63 (d, J=8.0 Hz, 2H), 7.49-7.55 (m, 4H), 7.21-7.26 (m, 5H), 5.69 (q, J=6.0 Hz, 1H), 1.63-1.65 (m, 2H), 1.47 (br, 3H), 1.26-1.28 (m, 2H).

IT075 was prepared following the general synthetic scheme for the synthesis of IT030, using

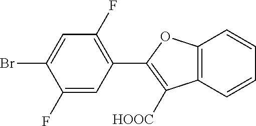 [SMILES: O=C(O)c1c(-c2cc(F)c(Br)cc2F)oc2ccccc12]

in place of XX-4 and

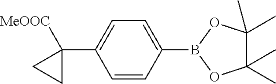 [SMILES: COC(=O)C1(c2ccc(B3OC(C)(C)C(C)(C)O3)cc2)CC1]

in place of XX-6. MS (ESI) m/z (M+Na)⁺576.0. Sodium salt IT075a: ¹H NMR (DMSO-d₆, 400 MHz): δ 9.73 (brs, 1H), 7.32-7.65 (m, 15H), 5.74-5.76 (m, 1H), 1.55 (br, 3H), 1.26 (br, 2H), 0.79 (br, 2H). MS (ESI) m/z (M+Na)⁺576.0.

### Example 13

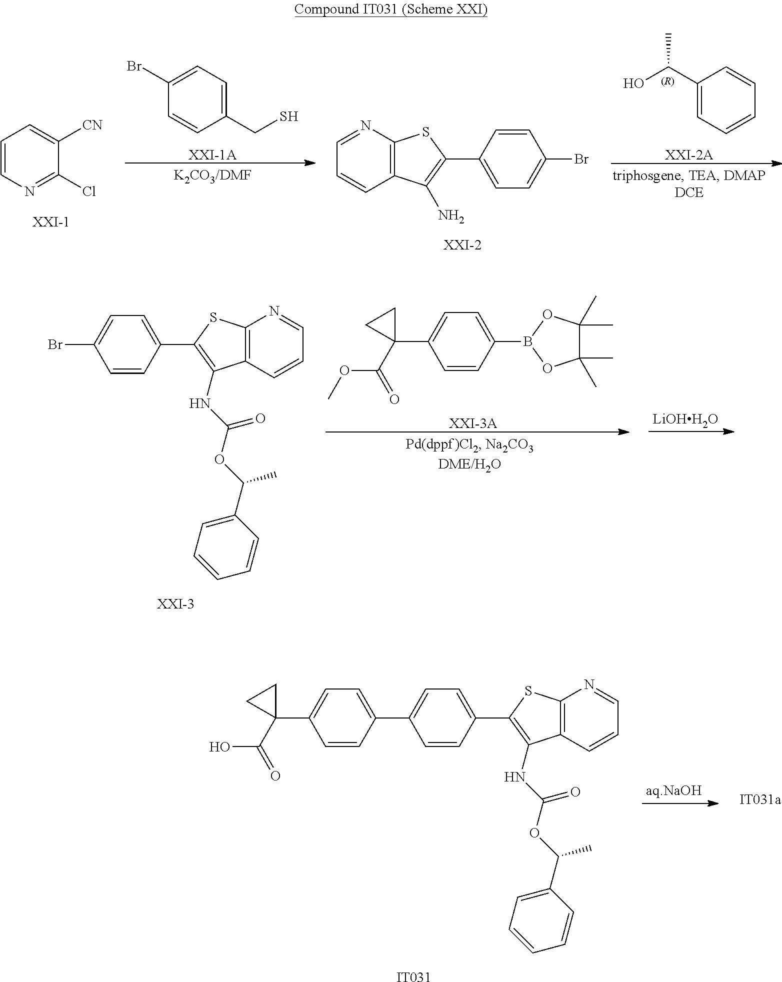

A mixture of XXI-1 (2.5 g, 18.04 mmol), XXI-1A (3.66 g, 18.04 mmol) and K₂CO₃ (9.98 g, 72.16 mmol) in 40 mL DMF was heated to 80° C. and stirred overnight. Then the reaction mixture was heated at 130° C. and stirred for additional 18 h. After cooled to it, the mixture was diluted with water, extracted with EtOAc. The combined organic layer was washed with brine, dried and concentrated. The resulting solid was washed with tert-butylmethylether to afford XXI-2 (3.5 g, yield 64%).

To a solution of XXI-2 (1 g, 3.28 mmol), TEA (2.3 mL, 16.4 mmol) and DMAP (1.49 g, 3.28 mmol) in 50 mL of dichloroethane was added triphosgene (0.97 g, 3.28 mmol) at 0° C. Then XXI-2A (2 g, 16.38 mmol) was added. The reaction mixture was stirred for 1 hour. The mixture was diluted with DCM, washed with H₂O, brine, dried and concentrated. The residue was purified by flash column chromatography over silica gel (PE:EA=4/1) to afford XXI-3 (1.1 g, yield 73%).

IT031 and its sodium salt IT031a were prepared following the similar procedures for the preparation of III-5, IT001 and IT001a. IT031a: ¹HNMR (400 MHz, DMSO-d₆) δ 9.61 (br, 1H), 8.60 (d, J=3.6 Hz, 1H), 7.91-7.92 (m, 1H), 7.68-7.72 (m, 4H), 7.36-7.56 (m, 10H), 5.75-7.56 (m, 1H), 1.55-1.56 (m, 3H), 1.22-1.23 (m, 2H), 0.72-0.73 (m, 2H). MS (ESI) m/z (M+H)⁺ 535.3.

### Example 14

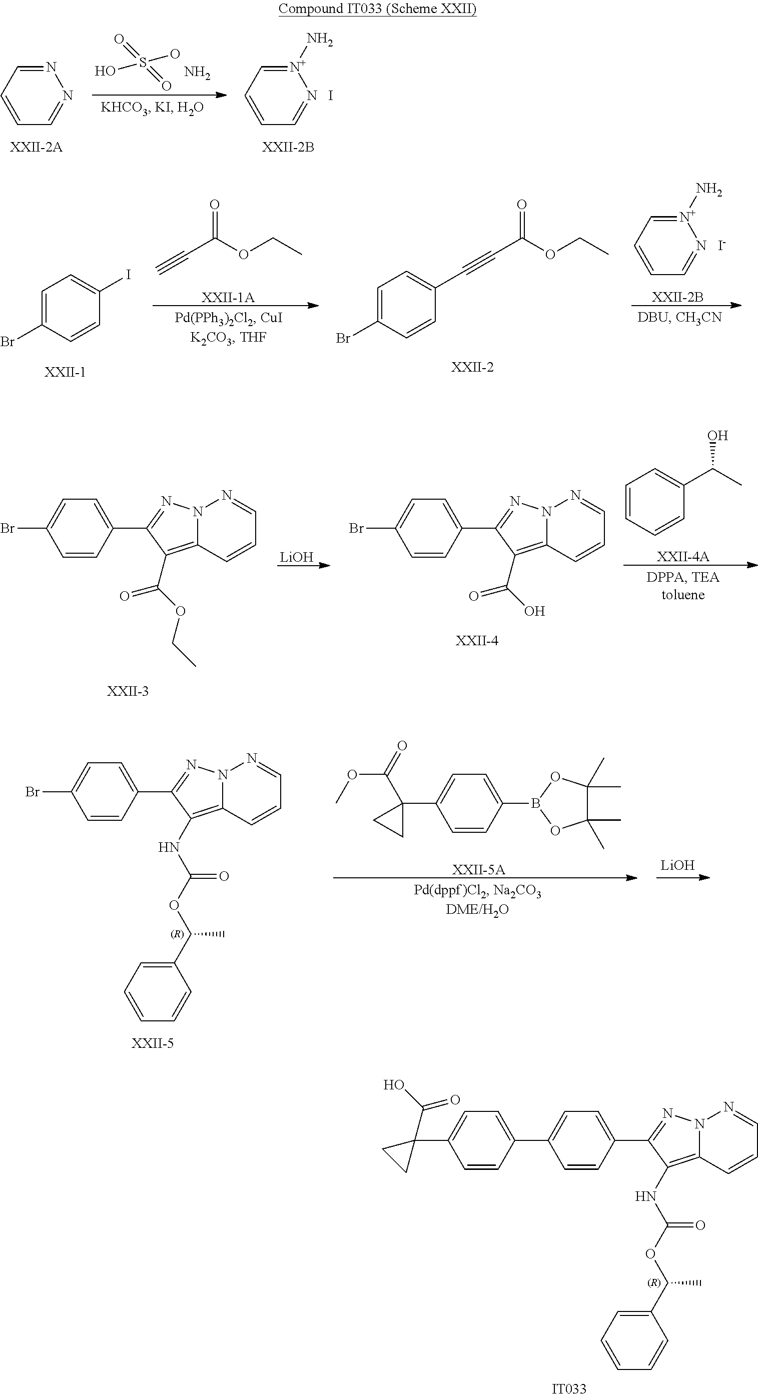

Aqueous KHCO₃ (2.4 mmol/mL) was added to a solution of Hydroxylamine-O-sulfonic acid (4.28 g, 37.9 mmol) in H₂O (8 mL) was cooled to 10° C. until pH to 5.0. Then XXII-2A (2 g, 25 mmol) was added in one portion and the reaction mixture was heated to 70° C. for 1 h. The pH was adjusted to 7.0 by the addition of aq. KHCO₃. The reaction was cooled to 40° C. and the mixture was allowed to stir for 1 h. Then KI (4.12 g, 25 mmol) in H₂O (8 mL) was added, and the solvent was removed in vacuo, followed by the addition of 5% methanol in ethanol (20 mL). The solids were collected by filtration and dried in vacuo to give crude XXII-2B (3.5 g, yield: 63.6%), which was used to next step directly.

To a solution of XXII-1 (10 g, 35.5 mmol) in THF (200 mL) was added K₂CO₃ (9.8 g, 71.0 mmol), CuI (270 mg, 1.42 mmol), Pd(PPh₃)₂Cl₂ (496 mg, 0.708 mmol) and XXII-1A (13.8 g, 140.8 mmol). The mixture was heated at 70° C. under N₂ for 12 h. After cooled to rt, water (50 mL) was added, and extracted with EtOAc. The organic layer was separated, dried, and concentrated, and the residue was purified by column chromatography (PE:EA=30:1) to afford XXII-2 (2.8 g, yield: 31.2%).

DBU (0.59 mL, 7.9 mmol) was added dropwise to a solution of XXII-2 (1 g, 3.95 mmol) and XXII-2B (1.76 g, 7.9 mmol) in CH₃CN (20 mL). The resulting mixture was stirred at 25° C. for 12 h. The solvent was removed under reduced pressure. The residue was dissolved in EtOAc and washed with H₂O. The organics were collected, dried with Na₂SO₄, filtered, and concentrated. The residue was purified by column chromatography (PE:EA=3:1) to give XXII-3 (148 mg, yield: 10.8%).

To a stirred solution of XXII-3 (148.0 mg, 0.4 mmol) in 15 mL of MeOH/H₂O/THF (v/v/v=1/1/1) was added LiOH.H₂O (90 mg, 2.2 mmol). After the addition, the solution was stirred at rt for 12 h. The mixture was concentrated in vacuo and adjusted pH to 4 with HCl (1N). The aqueous phase was extracted with EtOAc, washed with brine, dried over Na₂SO₄, and concentrated to afford crude XXII-4 (120.0 mg, yield 87.5%), which was used to next step directly.

The mixture of XXII-4 (220 mg, 0.69 mmol), XXII-4A (101 mg, 0.83 mmol), DPPA (228 mg, 0.83 mmol) and TEA (139 mg, 1.38 mmol) in toluene (10 mL) was stirred at 80° C. under nitrogen for 12 h. After cooled to rt, water was added. The organic layer was extracted with EtOAc, separated, dried, and concentrated. The residue was purified by chromatography on silica gel (PE:EA=1:1) to afford XXII-5 (156 mg, yield: 53.1%).

IT033 and its sodium salt IT033a were prepared following the similar procedures for the preparation of III-5, IT001 and IT001a. IT033: MS (ESI) m/z (M+H)⁺ 519.1. IT033a: ¹HNMR (DMSO-d₆, 400 MHz): δ 9.37 (s, 1H), 8.47 (s, 1H), 7.94-8.00 (m, 3H), 7.69-7.71 (m, 2H), 7.22-7.55 (m, 10H), 5.78 (q, 1H), 1.56 (d, J=5.2 Hz, 3H), 1.21 (br, 2H), 0.71 (br, 2H). MS (ESI) m/z (M+H)⁺ 519.1.

IT049 was prepared following the similar synthetic scheme of IT033 using 1-bromo-2,5-difluoro-4-iodobenzene in place of XXII-1 and ethyl 2-(4-bromo-2,5-difluorophenyl)pyrazolo[1,5-a]pyridine-3-carboxylate in place of XXII-3. In the last step Suzuki-coupling reaction, x-Phos and Pd₂(dba)₃ in dioxane were used instead of Pd(dppf)Cl₂ in DME/H₂O. IT049: MS (ESI) m/z (M+H)⁺ 554.18. Sodium salt IT049a: ¹H NMR (DMSO-d₆, 400 MHz): δ 9.15 (s, 1H), 8.70 (d, J=6.8 Hz, 1H), 7.27-7.53 (m, 13H), 0.97-0.99 (m, 1H), 5.72-5.74 (m, 1H), 1.51 (d, J=5.6 Hz, 2H), 1.17 (br, 3H), 0.71 (br, 2H). MS (ESI) m/z (M+H)⁺ 554.1.

IT061 was prepared following the similar synthetic scheme of IT033 using 1-bromo-2,5-difluoro-4-iodobenzene in place of XXII-1. IT061:MS (ESI) m/z (M+H)⁺ 555.1. Sodium salt IT061a: ¹HNMR (DMSO-d₆, 400 MHz): δ 9.40 (s, 1H), 8.52 (s, 1H), 8.11 (br, 1H), 7.25-7.58 (m, 12H), 5.74-5.75 (m, 1H), 1.52 (d, J=6.4 Hz, 3H), 1.26 (br, 2H), 0.80 (br, 2H). MS (ESI) m/z (M+H)⁺ 555.1.

### Example 15

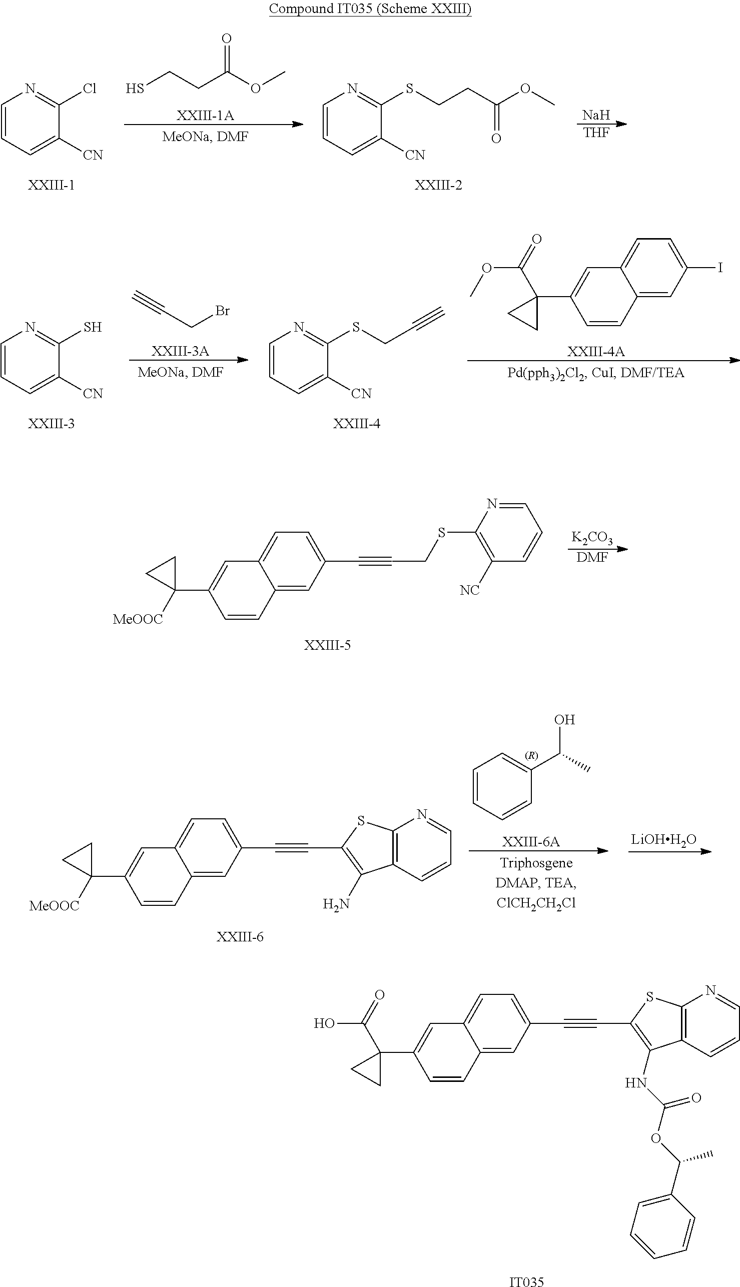

A mixture of sodium methoxide (3.48 g, 0.065 mol), XXIII-1A (18 g, 0.15 mol) and XXIII-1 (20.7 g, 0.15 mol) in dry DMF (30 mL) was stirred at rt for 24 hs, The mixture was poured into water and extracted with EA. The organic layer was washed with water, dried over Na₂SO₄, filtered, and evaporated to dryness. The residue was purified to afford XXIII-2 (9.0 g, yield 27%).

A mixture of XXIII-2 (3.0 g, 13.514 mmol), NaH (60%, 1.622 g) in THF (60 mL) was stirred at refluxed for 5 hs. After being cooled to rt, the excess hydride was destroyed by the addition of ice/water (5 mL). The solvent was removed in vacuo, and neutralized to pH=6.0 with 1N HCl. The precipitated solids was filtered and purified by prep-HPLC to afford XXIII-3 (1.2 g, yield 65.3%).

A solution of XXIII-3 (420 mg, 3.088 mmol) in dry DMF (5 mL) was treated with fresh sodium methoxide (183 mg, 3.397 mmol) at 0° C. Then XXIII-3A (400.8 mg, 3.397 mmol) in dry DMF (0.5 mL) was added dropwise to the mixture. The resulting mixture was stirred at rt overnight. The mixture were poured into water and extracted with EA. The organic layer was washed with water, dried over Na₂SO₄, filtered, and evaporated to dryness. The residue was purified by column chromatography (PE:EA=30:1) to give XXIII-4 (470 mg; yield: 87.5%).

XXIII-4A (811 mg, 2.241 mmol), Pd(PPh₃)₂Cl₂ (60.4 mg, 0.086 mmol), and CuI (32.8 mg, 0.172 mmol) were mixed with DMF (3 mL) and freshly distilled TEA (9 mL). Then a solution of the XXIII-4 (300 mg, 1.724 mmol) in DMF/TEA (3 mL/9 mL) was added slowly over the course of 1 h at rt. Once the addition is completed, TLC showed complete reaction. The mixture was poured into water, and extracted with EA. The extraction was washed with brine, dried over Na₂SO₄, filtered, and evaporated to dryness. The residue was purified by column chromatography (PE:EA=15:1) to give XXIII-5 (350 mg, yield: 51%).

To a solution of XXIII-5 (350 mg, 0.879 mmol) in dry DMF (4 mL) was added K₂CO₃ (485 mg, 3.518 mmol) at rt. The mixture was heated to 60° C. for 8 hs. The mixture was poured into water and extracted with EA. The extraction was washed with brine, dried over Na₂SO₄, filtered, and evaporated to dryness. The residue purified by column chromatography (PE:EA=15:1) to afford XXIII-6 (180 mg, yield: 51.4%).

To a solution of XXIII-6 (90 mg, 0.226 mmol) in 1,2-dichloroethane (2 mL) was added DMAP (27.6 mg, 0.226 mmol) and TEA (114 mg, 1.130 mmol). The mixture was stirred at 0° C. for 15 min., and then triphosgene (67 mg, 0.226 mmol) was added to the brown solution at 0° C. The mixture was stirred for 10 min, XXIII-6A (27.6 mg, 0.226 mmol) in 1,2-dichloroethane (1 mL) was added, and the reaction mixtures was stirred at rt for 2 h under N₂. The mixture was poured into water and extracted with EA. The organic layer was washed water, dried over Na₂SO₄, filtered, and evaporated to dryness. The residue was purified and further subject to hydrolysis by LiOH.H₂O (22.6 mg, 0.94 mmol at rt overnight. The mixture was poured into water, neutralized to pH=6.0, then extracted with EA. The organic layer was washed water, dried over Na₂SO₄, filtered, and evaporated to dryness. The residue was purified by preparative HPLC to afford IT035 (56 mg, yield 56.8%).

To a solution of IT035 (58.6 mg, 0.104 mmol) in MeOH/H₂O (v/v=3/1, 5 mL) was added aq. NaOH (2.48 mL, 0.05N, 0.104 mmol) at 0° C. The reaction mixture was stirred for 30 minutes. The reaction mixture was lyophilized to give IT035a. ¹H NMR (400 MHz, DMSO-d₆): 9.97 (s, 1H), 8.66 (dd, J=4.4 Hz, J=1.2 Hz, 1H), 8.01-8.08 (m, 2H), 7.85 (d, J=8.4 Hz, 1H), 7.67-7.77 (m, 2H), 7.57 (d, J=8.4 Hz, 1H), 7.34-7.48 (m, 6H), 7.27-7.33 (m, 1H), 5.87 (q, J=6.53 Hz, 1H), 1.58 (d, J=6.4 Hz, 3H), 1.22-1.27 (m, 2H), 0.77-0.82 (m, 2H). MS (ESI) m/z (M+H)⁺ 533.3.

### Example 16

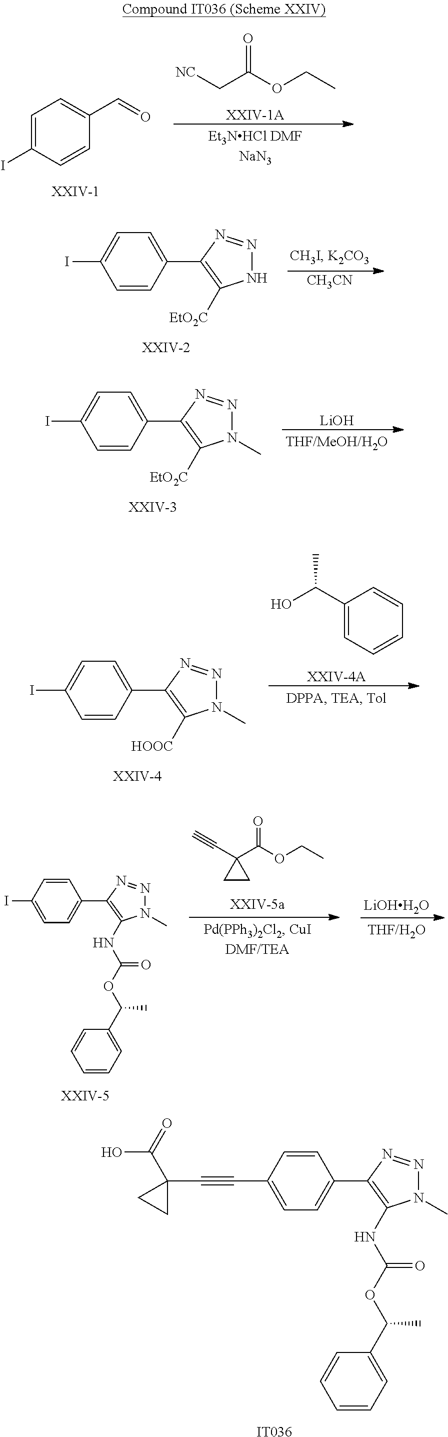

To a solution of XXIV-1 (12 g, 51.7 mmol) in DMF (180 mL) were added Et₃N.HCl (21.3 g, 155.1 mmol), NaN₃ (10.3 g, 163.5 mmol) and XXIV-1A (5.84 g, 51.7 mmol). The reaction mixture was heated at 70° C. for 18 hours under nitrogen protection. After completion of the reaction, the mixture was poured into water and extracted with EtOAc. The organic layers were dried over MgSO₄ and concentrated. The residue was purified by chromatography on silica gel (PE:EA=5:1) to afford XXIV-2 (6 g, yield: 33.8%).

To a solution of XXIV-2 (3 g, 8.752 mmol) in CH₃CN (50 mL), K₂CO₃ (2.41 g, 17.5 mmol), was added MeI (2.5 g, 17.5 mmol). The reaction mixture was stirred at rt overnight under nitrogen protection. Then CH₂Cl₂ and water was added, the organic layers were separated, dried over MgSO₄ and concentrated. The residue was purified by prep-HPLC to afford XXIV-3 (0.5 g, yield: 16.1%).

To a stirred solution of XXIV-3 (4.2 g, 14.2 mmol) in MeOH/THF/H₂O (v/v/v=1/2/1, 16 mL) was added LiOH (3 g, 71 mmol). After the addition, the solution was stirred overnight at rt. The solution was concentrated in vacuo, the aqueous layer was adjusted pH to 2, and extracted with EtOAc. The organic layer was separated, dried and concentrated to afford XXIV-4 (0.7 g, yield 76%).

To a solution of XXIV-4A (236 mg, 1.93 mmol) in dry toluene (8 mL) was added XXIV-4 (530 mg, 1.61 mmol), TEA (0.447 mL, 3.22 mmol) and DPPA (0.414 mL, 1.93 mmol). The reaction mixture was heated to 80° C. for 3 hours. The mixture was diluted with EtOAc, washed with brine, dried over Na₂SO₄, filtered and concentrated. The residue was purified by TLC (PE:EA=2:1) to give XXIV-5 (550 mg, yield: 76.1%).

IT036 was prepared from XXIV-5 in two steps following the similar procedure described the synthesis of IT001. MS (ESI) m/z (M+H)⁺ 431.1. Sodium salt IT036a: ¹HNMR (Methanol-d₄, 400 MHz) δ 7.66 (d, J=7.2 Hz, 2H), 7.36-7.45 (m, 9H), 5.84 (q, 1H), 3.91 (s, 3H), 1.57-1.64 (m, 5H), 1.43 (q, 2H). MS (ESI) m/z (M+H)⁺ 431.1.

### Example 17

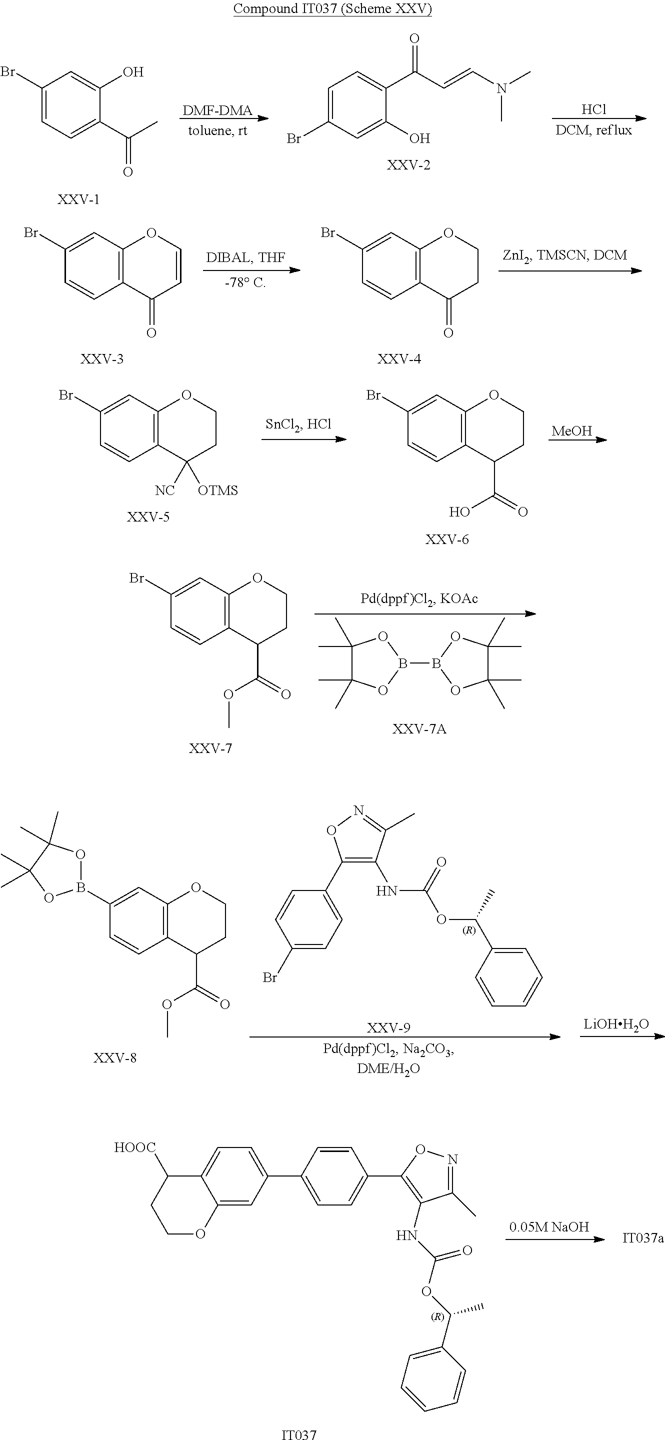

A solution of XXV-1 (5 g, 26 mmol) and DMF-DMA (12 mL, 52 mmol) in toluene (120 mL) was stirred at reflux for 4 hs. The mixture was concentrated and the residue was purified by flash chromatography (PE/EA=10/1) to give XXV-2 (2.7 g, yield: 40%) as a white solid. A solution of XXV-2 (4.5 g, 16.7 mmol) and conc. HCl (5 mL) in DCM (20 mL) was stirred at refluxed for 40 min. The organic layer was separated and the aqueous layer was extracted with DCM, the combined organic layer was washed with NaHCO₃ and brine, dried over Na₂SO₄ and concentrated to give XXV-3 (3.2 g, yield: 86%) as a white solid.

A solution of XXV-3 (2.9 g, 12.9 mmol) in THF (10 mL) was cooled to −78° C., DIBA1-H (24 mL, 24 mmol) was added and the resulting solution was stirred at −78° C. for 30 min., Then sat.NH₄Cl was added to quench the reaction, extracted with EA, washed with brine, dried over Na₂SO₄ and concentrated, the residue was purified by flash chromatography (PE/EA=1/1) to give XXV-4 (1.5 g, yield: 51.7%) as a yellow solid.

To a solution of XXV-4 (2.27 g, 10 mmol) was added the iodine (76.2 mg, 0.3 mmol), TMSCN (1.5 g, 15 mmol) in DCM (20 mL). The resulting solution was stirred at rt for another 24 hs. Then the NaHSO₃ (aq.) was added and extracted with DCM. The organic lays was evaporated in vacuum to afford the crude XXV-5, which was used to next step without purification.

To a solution of XXV-5 (3.27 g, 10 mmol) in HCl/HOAc (v/v=10 mL: 10 mL) was added SnCl₂ (6.6 g, 35 mmol) and the resulting solution was stirred at 80° C. for 18 hs. Then 10 mL of water was added and extracted with DCM. The combined organic layer was washed with 2N NaOH (aq.) and combined the aqueous lays. The aqueous lays was adjusted to pH (<3) with 5N HCl and extracted with DCM, and the combined organic layer was evaporated in vacuum to afford the crude XXV-6 without purification for next step.

To a solution of XXV-6 (1.88 g) in MeOH (20 mL) was added HCl (200 mg) and the resulting solution was stirred at 80° C. for another 4 hs. Then the solvent was evaporated and 50 mL of EA was added and washed with brine. The organic phase was dried over Na₂SO₄ and evaporated. The residue was purified by column chromatography to afford XXV-7 (1.5 g, yield: 76%).

XXV-8 was prepared from XXV-7 and XXV-7A following the similar procedure described in the synthesis of III-3.

IT037 was prepared from XXV-8 and XXV-9 in two steps following the similar procedure described the synthesis of III-5 and IT001. ¹H NMR (DMSO-d₆, 400 MHz): δ 12.62-12.91 (m, 1H), 9.11-9.51 (m, 1H), 7.71-7.88 (m, 5H), 7.38-7.52 (m, 4H), 7.31-7.37 (m, 2H), 7.21-7.29 (m, 1H), 7.11-7.17 (m, 1H), 5.49-5.94 (m, 1H), 4.07-4.45 (m, 3H), 4.07-4.45 (m, 3H), 3.76-3.93 (m, 1H), 2.01-2.29 (m, 7H), 1.45-1.70 (m, 3H). MS (ESI) m/z (M+H)⁺ 499.1. IT037a: ¹H NMR (Methanol-d₄, 400 MHz): δ 7.58-7.95 (m, 4H), 7.32-7.50 (m, 5H), 7.02-7.23 (m, 3H), 5.73-6.03 (m, 1H), 4.35-4.56 (m, 1H), 4.09-4.32 (m, 1H), 3.62-3.82 (m, 1H), 2.20 (s, 5H), 1.26-1.76 (m, 3H). MS (ESI) m/z (M+H)⁺ 499.1.

### Example 18

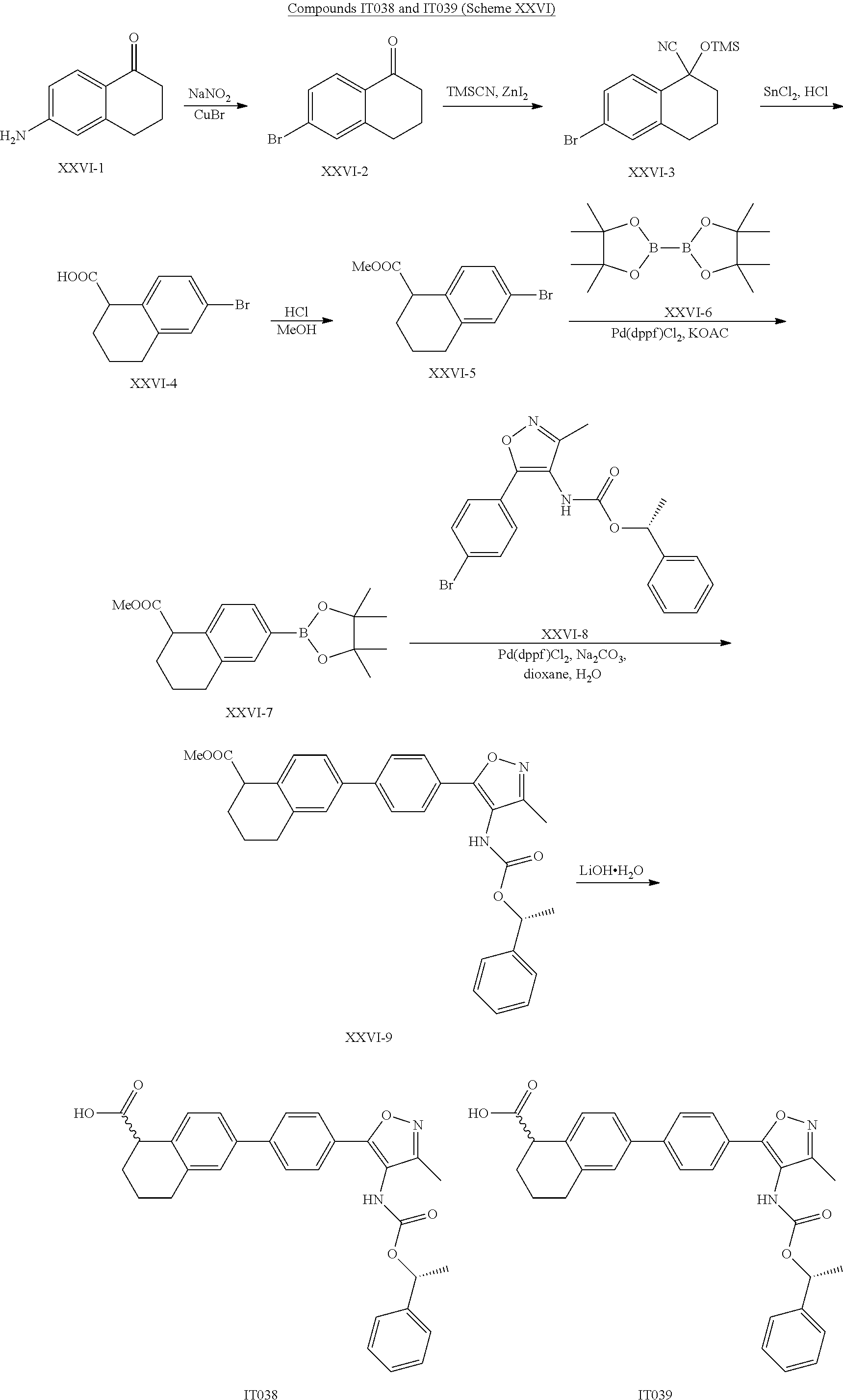

To a stirred solution of XXVI-1 (5.00 g, 0.03 mmol) in 15 mL of 40% aqueous HBr was added a solution of NaNO₂ (2.35 g, 0.034 mmol) in H₂O, maintaining the temperature at −5° C. under nitrogen. After the addition, the solution was stirred for another 0.5 hour. Then the resulting solution was warmed slowly to rt and stirred for another 3 hours. Then the solution was concentrated and the mixture was extracted with EtOAc. The organic layer was combined and washed with brine, dried over Na₂SO₄, concentrated in vacuo. The residue was purified by column chromatography on silica gel to afford XXVI-2 (3.5 g, yield: 52%).

The solution of XXVI-2 (3.50 g, 15.56 mmol), TMSCN (2.33 g, 23.34 mmol) and I₂ (0.40 g, 1.56 mmol) in DCM (30 mL) was stirred overnight at 25° C. under nitrogen. 20 mL of aqueous Na₂SO₃ was added, and the mixture was extracted with DCM. The organic layer was combined and washed with brine, dried over Na₂SO₄, concentrated in vacuo to afford 3.4 g of crude XXVI-3, which was used for next step without further purification.

XXVI-3 (3.4 g, 15.1 mmol) and SnCl₂ (10.0 g, 52.8 mmol) were added to a solution of HOAc and HCl (10 mL, V/V=1/1) under nitrogen. After the addition, the solution was heated to 90° C. under nitrogen for 24 hours. The mixture was extracted with DCM. The combined aqueous layers were washed with 2M NaOH. The combined aqueous layers were adjusted to pH=2 with 5 M HCl solution (10 mL). The acidic aqueous phase was extracted with EtOAc. The combined organic layer was washed with brine, dried over Na₂SO₄, concentrated to afford XXVI-4 (0.85 g, yield: 22%).

The solution of XXVI-4 (1.13 g, 5.02 mmol) and HCl (13.9 mg, catalyzed amount) in 10 mL of MeOH was heated to reflux under nitrogen for overnight. MeOH was removed in vacuo and the residue was partitioned between H₂O (20 mL) and EtOAc. The organic layer was washed with brine, dried over Na₂SO₄, and concentrated. The residue was purified by column chromatography on silica gel to afford XXVI-5 (0.80 g, yield: 55%).

XXVI-7 was prepared from XXVI-5 and XXVI-6 following the similar procedure described in the synthesis of III-3.

XXVI-7 was prepared from XXVI-7 and XXVI-8 following the similar procedure described the synthesis of III-5.

IT038 and IT039 racemic mixture: MS (ESI) m/z (M+H)⁺ 498.1. Their sodium salts IT038a and IT039a were obtained from SFC separation. IT038a: ¹H NMR (DMSO-d₆, 400 MHz): δ 9.39 (s, 1H), 7.74-7.79 (m, 4H), 7.33-7.44 (m, 8H), 5.76 (br, 1H), 2.73-2.76 (m, 2H), 2.08-2.13 (m, 4H), 1.96 (s, 1H), 1.76 (s, 1H), 1.56 (s, 3H). MS (ESI) m/z (M+H)⁺ 498.1. IT039a: ¹H NMR (Methanol-d₄, 400 MHz): δ 7.60-7.63 (m, 4H), 7.22-7.33 (m, 8H), 5.71 (br, 1H), 3.58-3.62 (m, 1H), 2.70-2.83 (m, 2H), 1.93-2.07 (m, 6H), 1.61-1.64 (m, 1H), 1.51 (s, 1H). MS (ESI) m/z (M+H)⁺ 498.1.

### Example 19

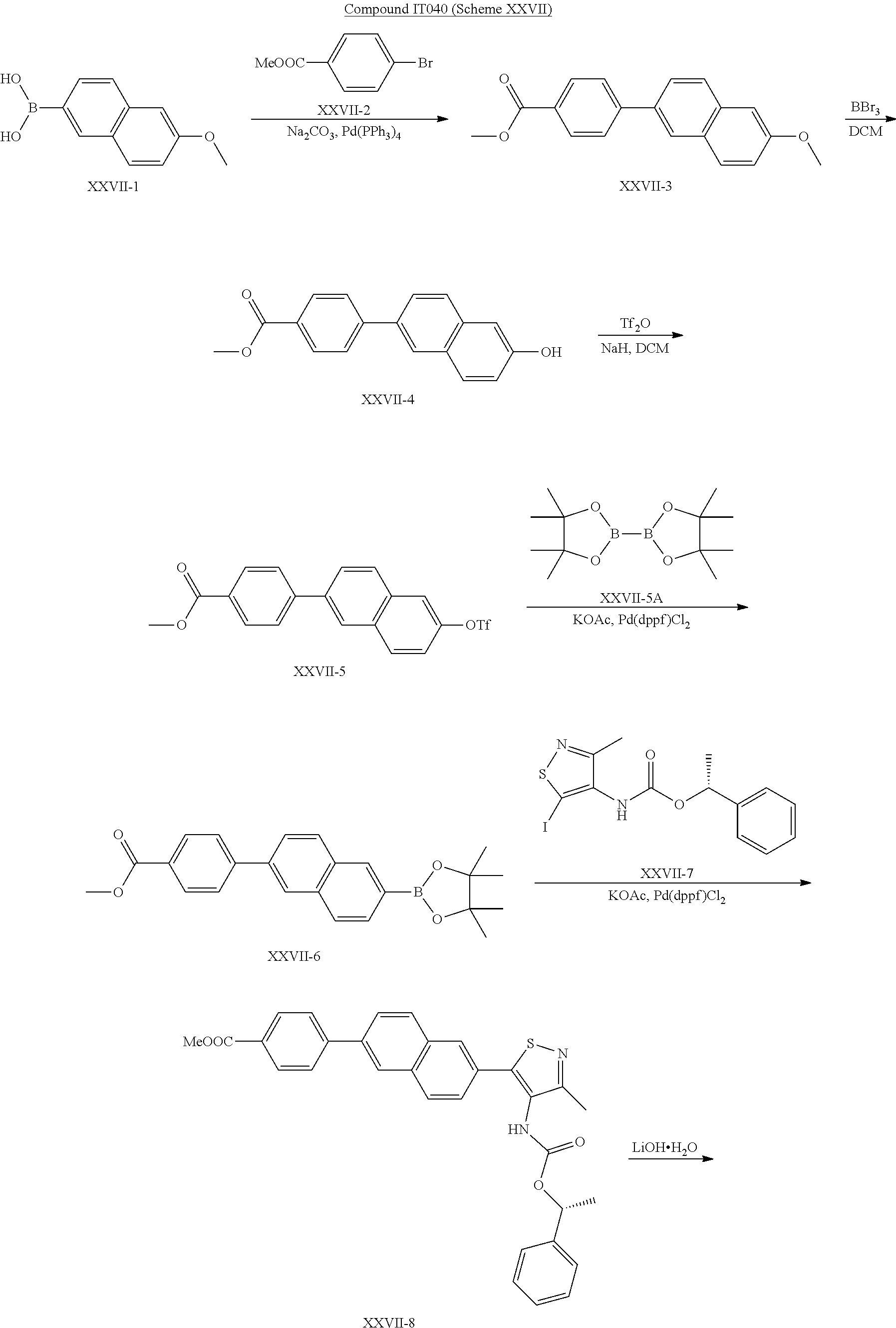

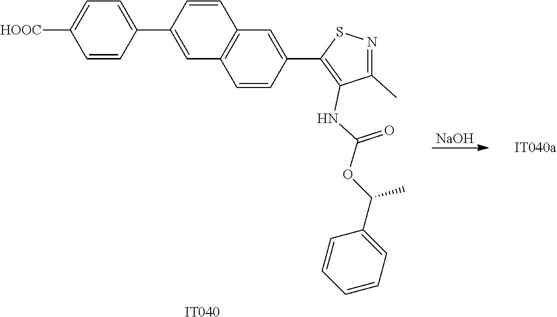

To a stirred solution of XXVII-1 (5.3 g, 26 mmol), XXVII-2 (5 g, 22 mmol), Na₂CO₃ (5.8 g, 55 mmol) in DME/H₂O (60 mL, v/v=5/1) was added Pd(PPh₃)₄ (1.27 g, 1.1 mmol) under nitrogen. Then the solution was heated to 110° C. for overnight. The solid formed was filtered and washed with water and dried in vacuo to obtain XXVII-3 (10 g, crude yield: 100%) as a brown solid.

To a stirred solution of XXVII-3 (300 mg, 1.03 mmol) in DCM (5 mL) was added BBr₃ (1 g, 4.1 mmol) dropwise at −78° C. Then it was stirred at rt for 6 hours. The mixture was quenched with H₂O. The organic layers were washed with brine, and concentrated under vacuo to give XXVII-4 (80 mg, yield: 28%).

To a stirred solution of XXVII-4 (300 mg, 1.08 mmol) in DCM (10 mL) was added NaH (129.6 mg, 3.24 mmol) under nitrogen at 0° C. Then the solution was warmed to rt. After 2 hours, Tf₂O (338 mg, 1.18 mmol) was added, and the mixture was stirred overnight. A saturated solution of NH₄Cl was added. The aqueous phase was extracted with DCM. The organic layer was combined and washed with brine, dried over Na₂SO₄, concentrated in vacuo to afford XXVII-5 (700 mg, crude).

XXVII-6 and XXVII-8 were prepared following the similar procedure in the synthesis of III-3 and III-5.

IT040 and IT040a were prepared following the similar procedure in the synthesis of IT001 and IT001a. IT040: MS (ESI) m/z (M+H)⁺ 509.1. ¹H NMR (Methanol-d₄, 400 MHz): δ 8.15-8.23 (m, 3H), 7.96-8.06 (m, 2H), 7.91 (d, J=7.6 Hz, 3H), 7.61 (br, 2H), 7.28-7.36 (m, 4H), 6.96 (br, 1H), 5.77 (br, 1H), 2.36 (s, 3H), 1.55 (s, 3H). IT040a: MS (ESI) m/z (M+H)⁺ 509.1. ¹H NMR (Methanol-d₄, 400 MHz): δ 9.26-9.55 (m, 1H), 8.31 (s, 1H), 7.92-8.24 (m, 6H), 7.76 (d, J=7.03 Hz, 2H), 7.66 (s, 1H), 7.37 (s, 4H), 5.74 (s, 1H), 2.31 (s, 3H), 1.51 (s, 3H).

### Example 20

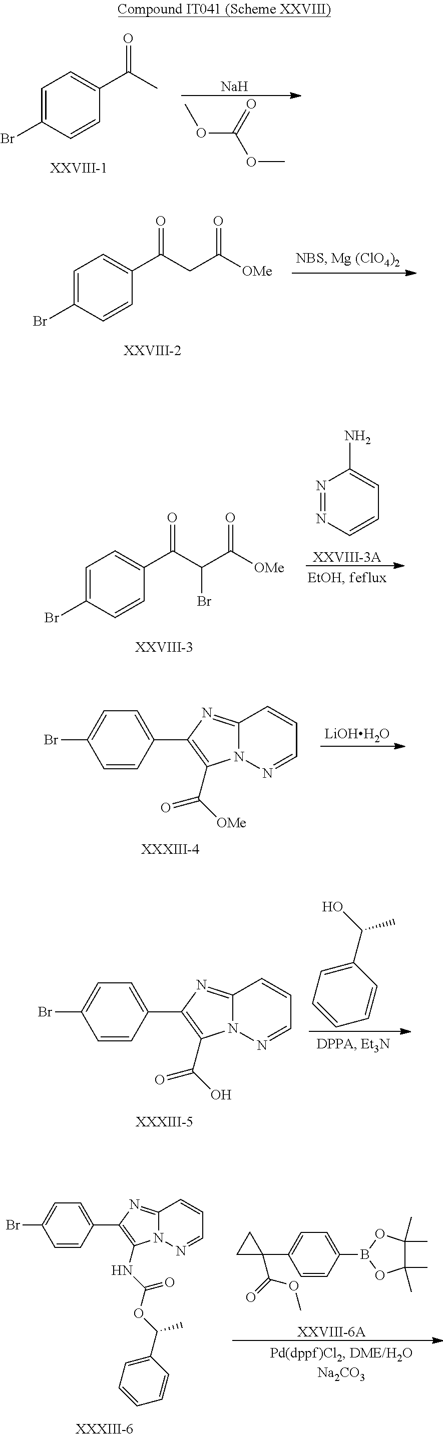

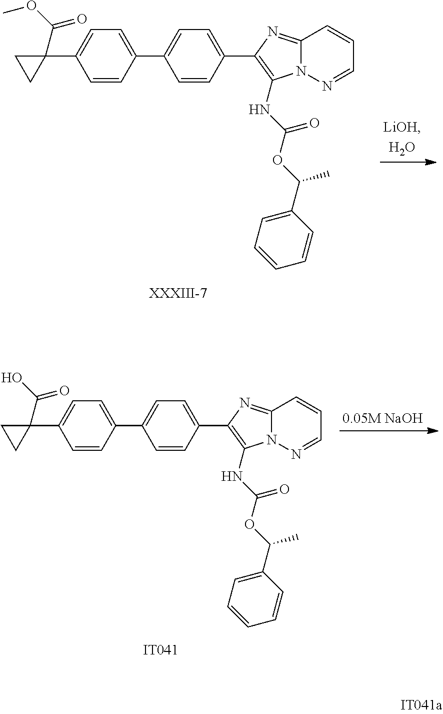

To a solution of XXVIII-1 (19.8 g, 0.1 mol) in THF (200 mL) was added NaH (8 g, 0.2 mol) at 0° C. The mixture was stirred at for 30 min. then added dimethyl carbonate (20 g, 0.3 mol). The solution was stirred at rt for 4 hour. Then NH₄Cl (aq.) was added to quench the solution and the resulting mixture was concentrated, washed and extracted with EA. The combined organic layers were washed with brine, dried over Na₂SO₄ and concentrated under vacuo. The crude was purified by column to afford XXVIII-2 (20.9 g, yield: 80.6%).

To a solution of XXVIII-2 (11.78 g, 45.25 mmol) in MeCN (120 mL) was added NBS (8.86 g, 49.78 mmol) and Mg(ClO₄)₂ (3.08 g, 13.57 mmol) and the resulting mixture was stirred at rt for 1 hour. After the reaction was complete, most of MeCN was removed under reduced pressure. Then 50 mL of H₂O was added and extracted with EA. The combined organic layers were washed with brine, dried over Na₂SO₄ and concentrated under reduced pressure. The crude was purified by column (PE/EA=10/1) to afford XXVIII-3 (9.2 g, yield: 60.33%).

To a solution of XXVIII-3 (4.6 g, 13.65 mmol) in EtOH (40 mL) was added XXVIII-3A (1.36 g, 14.33 mmol). Then the mixture was heated to reflux and stirred at the temperature for 48 hours. After removing most of EtOH under reduced pressure, 30 mL of water was added and extracted with EA. The combined organic layers were washed with brine, dried over Na₂SO₄ and concentrated. The crude was purified by column (PE/EA=10/1) to afford XXVIII-4 (1.26 g, yield: 27.8%).

To a solution of XXVIII-4 (1.26 g, 3.79 mmol) in 10 mL of MeOH/H₂O (v/v=5/1) was added LiOH.H₂O (0.96 g, 22.77 mmol). Then the mixture was heated to 60° C. overnight. MeOH was evaporated and another 10 mL of H₂O was added and extracted with EA. The combined organic layers were washed with brine, dried over Na₂SO₄ and concentrated. The crude product XXVIII-5 (1.1 g, yield: 91.7%) was used to next step directly.

To a solution of XXVIII-5 (900 mg, 2.84 mmol) in toluene (9 mL) was added (R)-1-phenylethanol (416 mg, 3.14 mmol), DPPA (937.8 mg, 3.41 mmol), Et₃N (574 mg, 5.68 mmol) under N₂ atmosphere. Then the mixture was heated to reflux for 2 hours. Then most of toluene was evaporated from the mixture and 10 mL of water was added and extracted with EA. The combined organic layers were washed with brine, dried over Na₂SO₄ and concentrated. The crude product was purified by column (PE/EA=10/1) to afford XXVIII-6 (880 mg, yield: 70%).

XXVIII-7 was prepared by reacting XXVIII-6 and XXVIII-6A following the similar procedure in the synthesis of III-5.

IT041 and IT041a were prepared following the similar procedure in the synthesis of IT001 and IT001a. IT041: MS (ESI) m/z (M+H)⁺ 519.2. IT041a: ¹H NMR (Methanol-d₄, 400 MHz): δ 8.46 (s, 1H), 7.95-8.01 (m, 2H), 7.56-7.66 (m, 4H), 7.40-7.46 (m, 5H), 7.28-7.29 (m, 2H), 5.86-5.87 (d, 1H), 1.65 (br, 3H), 1.50 (s, 2H), 1.05 (s, 2H). MS (ESI) m/z (M+H)⁺ 519.2.

IT043 was prepared following the similar synthetic scheme for the preparation of IT041 using pyrimidin-2-amine to replace XXVIII-3A. IT043: MS (ESI) m/z (M+H)⁺ 519.2. Sodium salt IT043a: MS (ESI) m/z (M+H)⁺ 519.2. ¹H NMR (DMSO-d₆, 400 MHz): δ 8.56 (br, 1H), 8.41 (br, 1H), 7.93 (br, 2H), 7.32-7.63 (m, 11H), 7.04-7.06 (m, 1H), 5.85 (br, 1H), 1.63 (br, 1H), 1.42-1.44 (m, 2H), 0.95-0.96 (m, 2H).

### Example 21

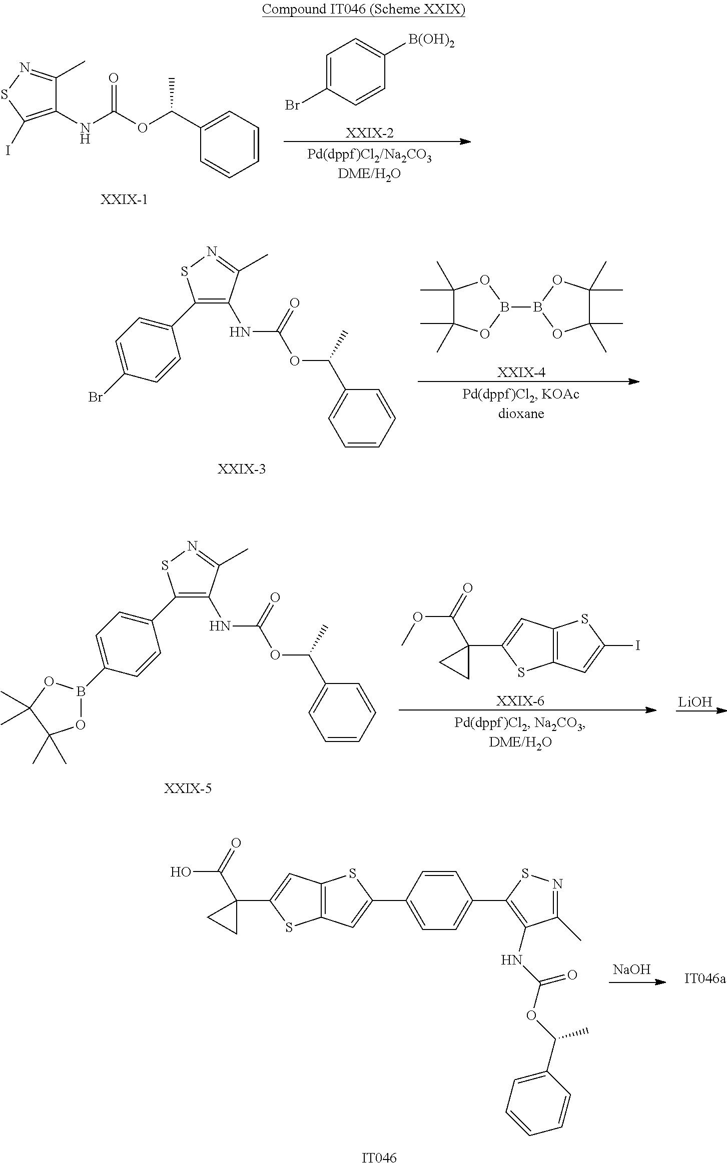

The mixture of XXIX-1 (6.60 g, 17.0 mmol), XXIX-2 (3.62 g, 17.8 mmol), Na₂CO₃ (4.5 g, 42.5 mmol) and Pd(dppf)Cl₂ (124 mg, 0.17 mmol) in DME/H₂O (150 mL, v/v=3/1) was heated to reflux under nitrogen for 12 hours. After concentrated, the residue was partitioned between H₂O and EA, the aqueous phase was extracted with DCM, and the combined organic layer was washed with brine, dried over MgSO₄, concentrated. The residue was purified by column chromatography on silica gel (PE:EA=10:1) to afford XXIX-3 (4.5 g, yield: 63.5%).

The mixture of XXIX-3 (4.5 g, 11 mmol), XXIX-4 (2.93 g, 11.6 mmol), KOAc (2.97 g, 27.5 mmol) and Pd(dppf)Cl₂ (80.4 mg, 0.11 mmol) in dioxane (150 mL, v/v=3/1) was heated to reflux under nitrogen for 12 hours. After concentrated, the residue was partitioned between H₂O and EA, the aqueous phase was extracted with EA, and the combined organic layer was washed with brine, dried over MgSO₄, concentrated. The residue was purified by column chromatography on silica gel (PE:EA=10:1) to afford XXIX-5 (3.8 g, yield: 76.1%).

IT046 was prepared by reacting XXIX-5 and XXIX-6 following the similar procedure for the preparation of XXIX-3 followed by LiOH hydrolysis. Sodium salt IT046a: ¹H NMR (DMSO-d₆, 400 MHz): δ 9.29 (s, 1H), 7.84 (s, 1H), 7.68 (d, J=8.0 Hz, 2H), 7.49 (d, J=8.0 Hz, 2H), 7.31-7.39 (m, 5H), 6.91 (s, 1H), 5.71-5.72 (m, 1H), 2.23 (s, 3H), 1.51 (d, J=6.4 Hz, 3H), 1.45 (br, 2H), 1.01 (br, 2H). MS (ESI) m/z (M+H)⁺ 561.0.

IT050 was prepared following the synthetic scheme of IT046 using the corresponding carbamate (R)-1-phenylethyl (4-(4-bromophenyl)-1-methyl-1H-1,2,3-triazol-5-yl)carbamate in place of XXIX-3. IT050: MS (ESI) m/z (M+H)⁺ 545.0. Sodium salt IT050a: ¹HNMR (DMSO-d₆, 400 MHz): δ 10.04 (br, 1H), 7.77 (s, 1H), 7.74 (d, J=8.4 Hz, 2H), 7.66 (d, J=8.4 Hz, 2H), 7.36 (m, 5H), 6.88 (s, 1H), 5.77 (q, J=6.4 Hz, 1H), 3.84 (s, 3H), 1.53 (d, J=6.4 Hz, 3H), 1.44-1.45 (m, 2H), 0.97-0.98 (m, 2H). MS (ESI) m/z (M+H)⁺ 545.1.

IT051 was prepared following the synthetic scheme of IT046 using the corresponding carbamate (R)-1-phenylethyl (1-(4-bromophenyl)-4-methyl-1H-1,2,3-triazol-5-yl)carbamate in place of XXIX-3. Sodium salt IT051a: ¹H NMR (400 MHz, DMSO-d₆): δ 7.83 (s, 1H), 7.75 (d, J=8.4 Hz, 2H), 7.62 (d, J=8.8 Hz, 2H), 7.25-7.34 (m, 5H), 6.91 (s, 1H), 5.69-5.64 (q, 1H), 2.09 (s, 3H), 1.47-1.48 (m, 2H), 1.40 (d, J=6.0 Hz, 3H), 1.00-1.01 (m, 2H). MS (ESI) m/z (M+H)⁺545.1.

IT056 was prepared following a modified synthetic scheme of IT046 by reacting the corresponding (R)-1-phenylethyl (4-(4-aminophenyl)-1-methyl-1H-pyrazol-5-yl)carbamate in place of XXIX-3 in the presence of benzoyl peroxide (BPO), tert-butyl nitrite and acetonitrile. IT056: MS (ESI) m/z (M+H)⁺ 544.0. Sodium salt IT056a: ¹H NMR (Methanol-d₄, 400 MHz): δ 7.74 (s, 1H), 7.52-7.56 (m, 3H), 7.33-7.42 (m, 6H), 7.09-7.16 (m, 2H), 5.84 (d, J=5.6 Hz, 1H), 3.71 (s, 3H), 1.59-1.62 (m, 5H), 1.21-1.23 (m, 2H). MS (ESI) m/z (M+H)⁺ 544.1.

IT067 was prepared following a modified synthetic scheme of IT046 using (R)-1-phenylethyl (1-(4-bromo-2,5-difluorophenyl)-4-methyl-1H-1,2,3-triazol-5-yl)carbamate (XX-5) in place of XXIX-3. The preparation of XX-5 was described in the synthesis of IT030. IT067: MS (ESI) m/z (M+H)⁺ 581.0. Sodium salt IT067a: ¹H NMR (DMSO-d₆, 400 MHz): δ 7.85 (s, 1H), 7.71 (br, 1H), 7.37-7.38 (m, 1H), 7.24-7.25 (m, 4H), 7.19-7.21 (m, 1H), 5.67-5.72 (q, 1H), 2.28 (s, 3H), 1.64-1.68 (m, 2H), 1.49 (br, 3H), 1.25-1.28 (m, 2H). MS (ESI) m/z (M+H)⁺ 581.0.

IT071 was prepared following the synthetic scheme of IT046 using the corresponding (R)-1-phenylethyl (4-(4-bromo-2,5-difluorophenyl)-1-methyl-1H-1,2,3-triazol-5-yl)carbamate in place of XXIX-3. IT071: ¹H NMR (Methanol-d₄, 400 MHz): δ 7.77 (s, 1H), 7.25-7.48 (m, 8H), 5.78 (s, 1H), 3.94 (s, 3H), 1.74-1.75 (m, 2H), 1.59 (s, 3H), 1.45-1.46 (m, 2H). MS (ESI) m/z (M+H)⁺ 580.9. IT071a: ¹H NMR (DMSO-d₆, T=80, 400 MHz): δ 7.89 (s, 1H), 7.54-7.61 (m, 1H), 7.51-7.52 (m, 1H), 7.29-7.35 (m, 5H), 7.22 (s, 1H), 5.77 (q, J=6.4 Hz, 1H), 3.91 (s, 3H), 1.61-1.64 (m, 2H), 1.51 (d, J=6.4 Hz, 3H), 1.26-1.27 (m, 2H). MS (ESI) m/z (M+H)⁺ 581.0.

### Example 22

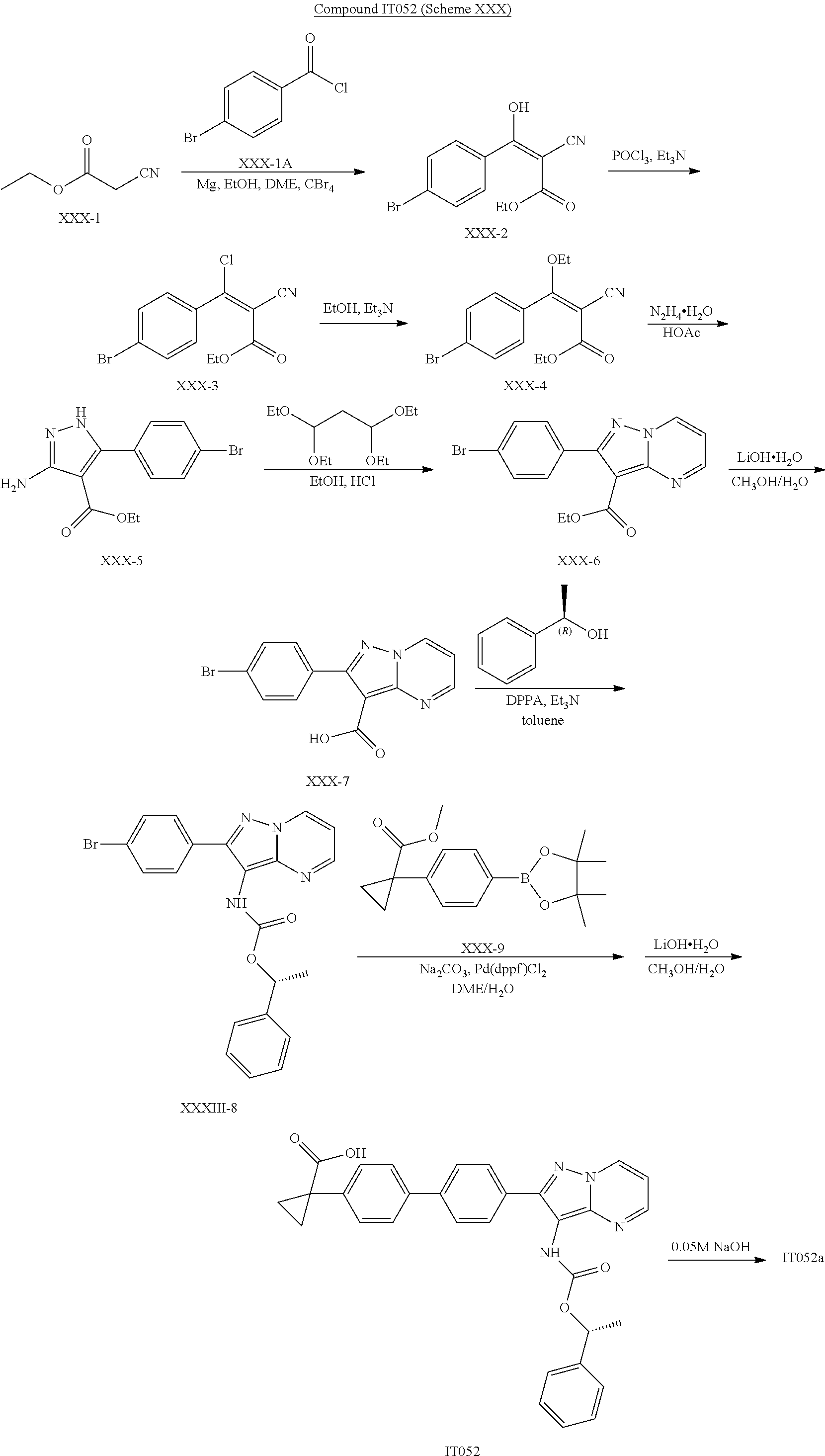

To a stirred solution of Mg (2.1 g, 0.09 mol) in dry EtOH (50 mL) and DME (50 mL) was added CBr₄ (176.1 mg, 0.53 mol). The mixture was heated to 90° C. for overnight. After being cooled to rt, the mixture was evaporated. The magnesium ethoxide formed was dissolved in DME (50 mL) and XXX-1 (10 g, 0.09 mol) was added at 20° C. The solution was cooled to 0° C. and p-bromobenzoyl chloride (19.4 g, 0.09 mol) was added below 40° C. The solution was stirred for 15 hs at rt. The solvent was evaporated and aq. HCl (5 M, 30 mL) was added. The mixture was extracted with DCM. The combined organic layers were washed with water, dried, and concentrated under vacuo. The residue was purified by column chromatography on silica gel (PE:EA=1:1) to give XXX-2 (16 g, yield: 61%).

To a stirred solution of XXX-2 (32.5 g, 0.11 mol), POCl₃ (290.7 mg, 0.72 mmol) in DCM (100 mL) was added dropwise Et₃N (37 g, 0.24 mol). Then the solution was heated to reflux for 15 hs. The solution was extracted with aq.HCl (5 M, 100 mL). The solvent was evaporated and the reminder was dissolved in EtOAc and washed with aq.HCl (5 M) and sodium bicarbonate solution. The organic layers were dried and concentrated under vacuo. The residue was purified by column chromatography on silica gel (PE:EA=10:1) to afford XXX-3 (20 g, yield: 58%).

To a stirred solution of XXX-3 (5 g, 0.016 mol) in EtOH (50 mL) was added Et₃N (8.03 g, 0.08 mol). The mixture was heated to 50° C. for 4 hs. EtOH was removed in vacuo and the residue was purified by column chromatography on silica gel (PE:EA=5:1) to afford XXX-4 (1.7 g, yield: 33%).

To a stirred solution of XXX-4 (1.7 g, 5.25 mmol) in HOAc (50 mL) was added N₂H₄.H₂O (0.5 g, 10.5 mmol). The solution was heated to reflux for 1.5 hours. EtOH was removed in vacuo. Brine was added to the residue and extracted with DCM. The combined organic layers were dried and concentrated under vacuo. The residue was purified by column chromatography on silica gel (PE:EA=1:1) to give XXX-5 (680 mg, yield: 42.5%).

To a solution of XXX-5 (680 mg, 2.19 mmol) in 30 mL MeOH was treated with 1,1,3,3-tetraethoxypropane (723.9 mg, 3.29 mmol) and lmL HCl. The solution was heated to 60-80° C. for 3 hs. The solvent was removed in vacuo and the residue was purified by column chromatography on silica gel (PE:EA=1:1) to give XXX-6 (314 mg, yield: 41.3%).

XXX-7, XXX-8, IT052 and IT052a were prepared following the similar procedure described in the synthesis of XXII-4, XXII-5, IT033 and IT033a. IT052: MS (ESI) m/z (M+H)⁺ 519.2. IT052a: ¹HNMR (Methanol-d₄, 400 MHz): δ 8.88-8.89 (br, 1H), 8.53 (br, 1H), 7.94 (d, J=8.0 Hz, 2H), 7.60-7.65 (m, 4H), 7.47-7.48 (m, 4H), 7.27-7.41 (m, 3H), 7.04 (br, 2H), 5.84 (br, 1H), 1.62 (br, 5H), 1.25 (br, 2H). MS (ESI) m/z (M+H)⁺ 519.2.

### Example 23

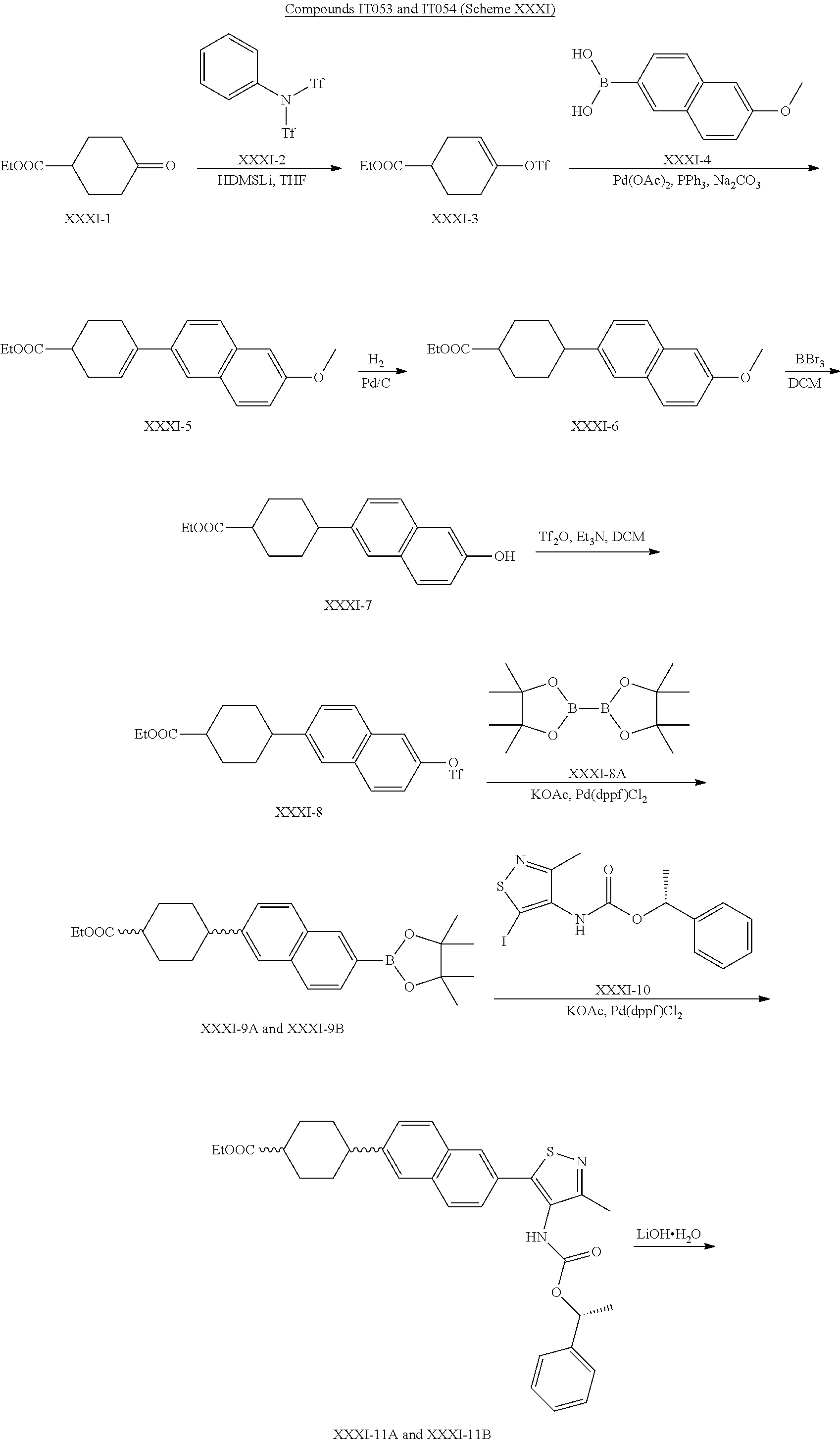

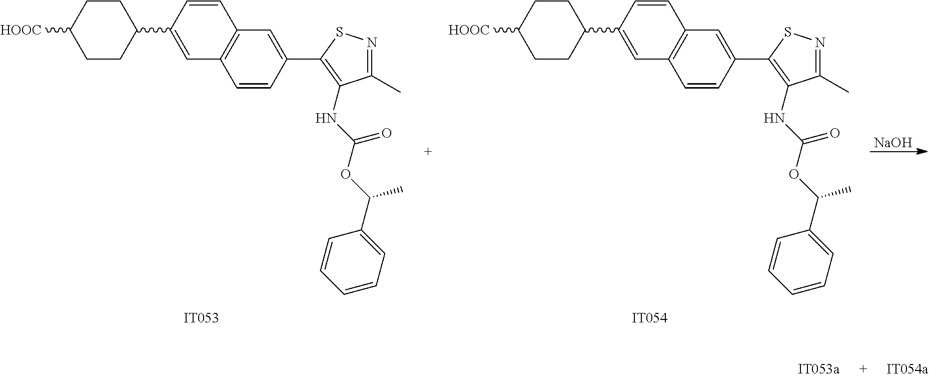

To a solution of XXXI-1 (5 g, 29.4 mmol) in THF (50 mL) was added LiHMDS (30.9 mL, 30.9 mmol) at −78° C. The solution was stirred at −78° C. for 1 h, then XXXI-2 (11 g, 30.9 mmol) in THF (50 mL) was added. The cooling bath was removed after stirring for 30 mins, the solution was stirred at rt overnight. The reaction was quenched with 1N NaHSO₃ and the solvent was evaporated. The residue was pardoned between EA and water. The organic layer was washed with 0.5 N NaOH, NH₄Cl and brine, dried over Na₂SO₄ and concentrated to afford XXXI-3 (10 g, crude yield: 100%).

To a stirred solution of XXXI-3 (10 g, 33.1 mmol), XXXI-4 (6.69 g, 33.1 mmol), Na₂CO₃ (7.02 g, 66.2 mmol) and PPh₃ (0.74 g, 3.31 mmol) in EtOH/toluene (120 mL, V/V=1/3) was added Pd(OAc)₂ (0.87 g, 3.31 mmol) under N₂. The mixture was purged with nitrogen for 5 minutes and heated to reflux for 2 hs. After being cooled to rt, the mixture was diluted with water and extracted with EtOAc. The combined organic layers were washed with brine, and concentrated under vacuo. The residue was purified by column chromatography on silica gel (PE:EA=10:1) to give XXXI-5 (5 g, yield: 50%).

To a stirred solution of XXXI-5 (4 g, 12.9 mmol) in MeOH (80 mL) was added Pd/C (2 g, 50%). Then the suspension was degas sed under vacuum and purged with H₂ (50Psi) at rt for 3 hs. Then the solution was filtered and evaporated in vacuo to give XXXI-6 (3.5 g, yield: 88%).

To a stirred solution of XXXI-6 (1 g, 3.2 mmol) in DCM (10 mL) was added BBr₃ (3.1 g, 12.8 mmol) dropwise at −78° C. Then it was stirred at rt for 4 hs. The mixture was quenched with H₂O. The organic layers were washed with brine, and concentrated under reduced pressure to give XXXI-7 (0.94 g, yield: 100%).

To a stirred solution of XXXI-7 (0.94 g, 3.15 mmol) and Et₃N (0.96 g, 9.46 mmol) in DCM (10 mL) was added Tf₂O (1.08 g, 3.8 mmol) under nitrogen at 0° C. and the mixture was stirred overnight. 10 mL of H₂O was added and the aqueous phase was extracted with DCM. The organic layer was combined and washed with brine, dried over Na₂SO₄, concentrated in vacuo to afford XXXI-8 (1.35 g, crude yield: 100%).

XXXI-9A, XXXI-9B, XXXI-11A and XXXI-11B were prepared following the similar procedure described in the synthesis of III-3.

IT053, IT054 and their sodium salts IT053a, IT054a were prepared following the similar procedure described in the synthesis of IT001 and IT001a. IT053 and IT054: MS (ESI) m/z (M+H)⁺ 515.2.

IT053a: MS (ESI) m/z (M+H)⁺ 515.1. ¹HNMR (DMSO-d₆, 400 MHz) δ 9.35 (br, 1H), 8.02 (s, 1H), 7.91 (d, J=8.4 Hz, 1H), 7.79 (d, J=8.0 Hz, 1H), 7.69 (s, 1H), 7.55 (d, J=8.4 Hz, 1H), 7.24-7.47 (m, 5H), 7.09 (br, 1H), 5.72 (br, 1H), 2.61-2.69 (m, 1H), 2.16-2.29 (m, 6H), 1.75-1.82 (m, 2H), 1.61-1.64 (m, 2H), 1.44-1.52 (m, 5H).

IT054a: MS (ESI) m/z (M+H)⁺ 515.1. ¹HNMR (Methanol-d₄, 400 MHz) δ 7.94 (s, 1H), 7.82 (d, J=7.6 Hz, 1H), 7.68-7.70 (m, 2H), 7.42-7.49 (m, 2H), 7.25-7.32 (m, 4H), 7.00 (s, 1H), 5.75 (br, 1H), 2.71 (s, 1H), 2.23-2.32 (m, 4H), 1.99-2.25 (m, 4H), 1.52-1.66 (m, 7H).

### Example 24

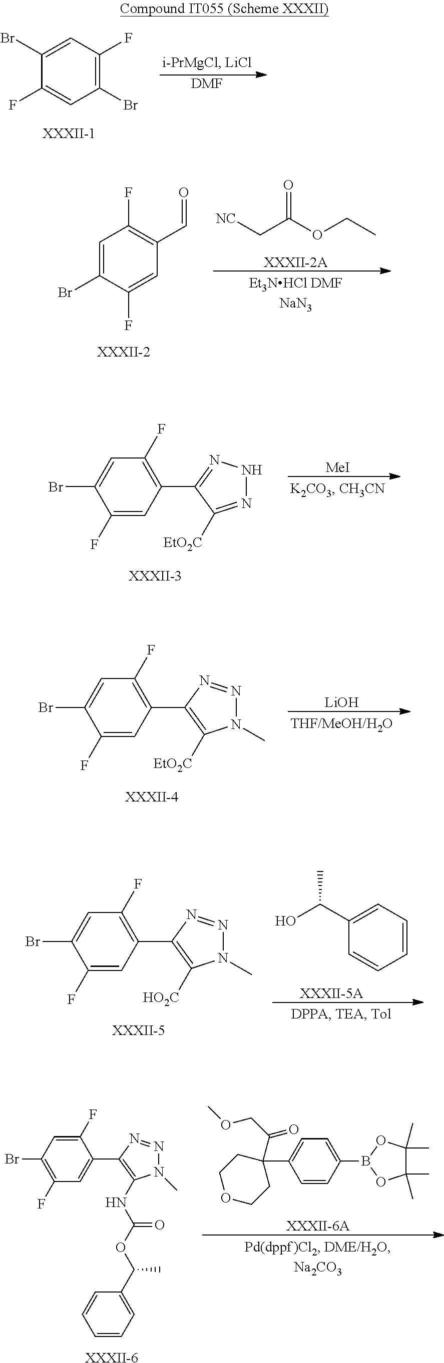

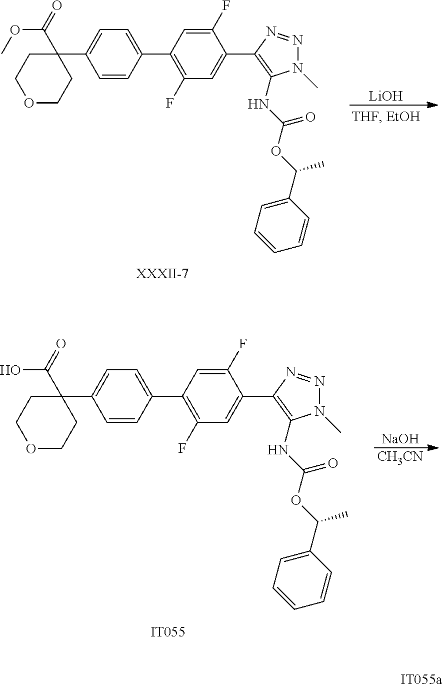

To a stirred solution of XXXII-1 (12 g, 44.1 mmol) in THF (150 mL) was added dropwise of XXXII-1A (44.1 mmol, 34 mL, 1.3 M) at −40° C. After stirred 1 h at −40° C., DMF (64 g, 882 mmol) was added and the mixture was stirred overnight. NH₄Cl (aq., 2M) was added and the mixture was extracted with EtOAc. The organic phase was dried with Na₂SO₄. The solvent was removed in vacuo and the residue was purified by column chromatography (PE/EA=10/1) to afford XXXII-2 (6.5 g, yield: 66.7%).

To a solution of XXXII-2 (3 g, 13.6 mmol) in DMF (30 mL), Et₃N.HCl (4.66 g, 34 mmol) were added NaN₃ (2.4 g, 40.8 mmol) and XXXII-2A (1.53 g, 13.6 mmol). The reaction mixture was heated at 70° C. and stirred overnight under nitrogen protection. After completion of the reaction, the mixture was poured into water and extracted with EtOAc. The organic phase was dried with Na₂SO₄. The solvent was removed in vacuo and the residue was purified by column chromatography (PE:EA=3:1) to afford XXXII-3 (0.5 g, yield: 11%).

XXXII-4, XXXII-5 and XXXII-6 were prepared following the similar procedure described in the synthesis of XXIV-3, XXIV-4 and XXIV-5.

XXXII-7, IT055, and IT055a were prepared following the similar procedure described in the synthesis of III-5, IT001 and IT001a. IT055: MS (ESI) m/z (M+H)⁺ 562.5. IT055a: ¹HNMR (DMSO-d₆ 400 MHz) δ 7.55-7.49 (m, 5H), 7.35-7.28 (m, 6H), 5.78 (q, 1H), 3.90 (s, 3H), 3.82-3.78 (m, 2H), 3.60-3.55 (m, 2H), 2.51-2.43 (m, 2H), 1.87-1.80 (m, 2H), 1.49 (d, J=6.0 Hz, 3H). MS (ESI) m/z (M+H)⁺ 563.1.

### Example 25

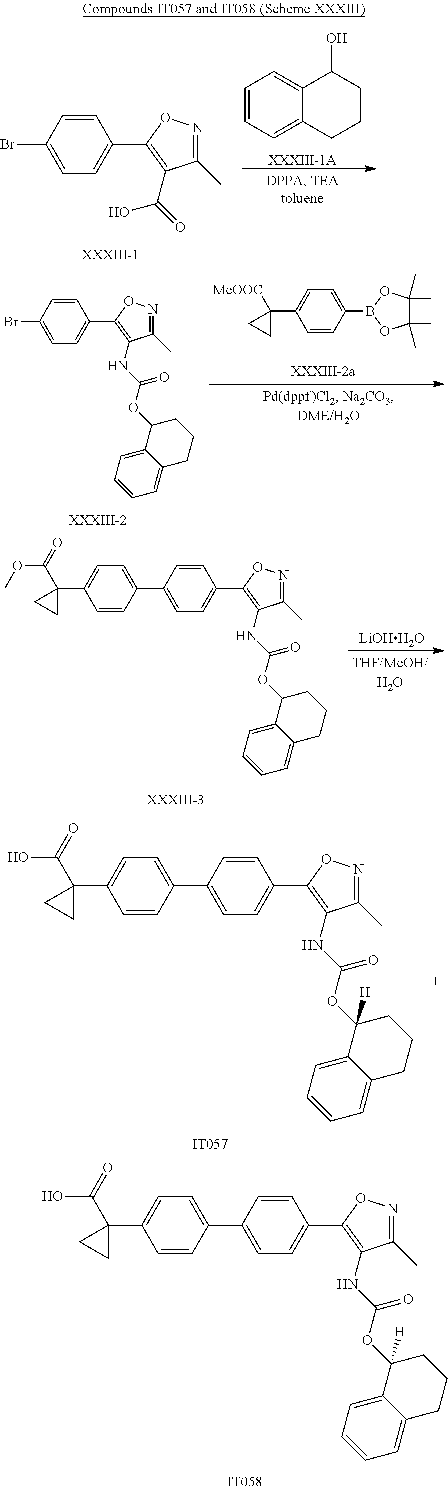

To a solution of XXXIII-1 (1 g, 3.6 mmol) in dry toluene (10 mL) was added XXXIII-1A (0.639 g, 4.3 mmol), TEA (0.763 g, 7.2 mmol) and DPPA (1.18 g, 4.3 mmol). The reaction mixture was heated to 80° C. for 6 h. The mixture was diluted with EtOAc, washed with brine, dried over Na₂SO₄, filtered and concentrated. The residue was purified by column chromatography (PE:EA=5:1) to give XXXIII-2 (1.3 g, yield 84.9%).

XXXIII-3 prepared by reacting XXXIII-2 with XXXIII-2A following the similar procedure described in the synthesis of III-5,

IT057 and IT058 were prepared following the similar procedure described in the synthesis of IT001, followed by chiral separation by SFC. MS (ESI) m/z (M+H)⁺ 509.1.

Sodium salt IT057a: ¹HNMR (400 MHz, DMSO-d₆) δ 9.25 (s, 1H), 7.80-7.87 (m, 4H), 7.57 (d, J=8.0 Hz, 2H), 7.36 (d, J=8.0 Hz, 2H), 7.05-7.24 (m, 4H), 5.85 (br, 1H), 2.77-2.89 (m, 2H), 2.21 (s, 3H), 1.85-2.09 (m, 4H), 1.22 (br, 2H), 0.73 (br, 2H). MS (ESI) m/z (M+H)⁺ 509.2.

Sodium salt IT058a: ¹H NMR (400 MHz, DMSO-d₆) δ 9.26 (s, 1H), 7.79-7.87 (m, 4H), 7.56 (d, J=8.0 Hz, 2H), 7.25 (d, J=8.0 Hz, 2H), 7.06-7.19 (m, 4H), 5.85 (s, 1H), 2.76-2.88 (m, 3H), 2.21 (s, 3H), 1.85-2.03 (m, 4H), 1.22 (br, 2H), 0.73 (br, 2H). MS (ESI) m/z (M+H)⁺ 509.2.

### Example 26

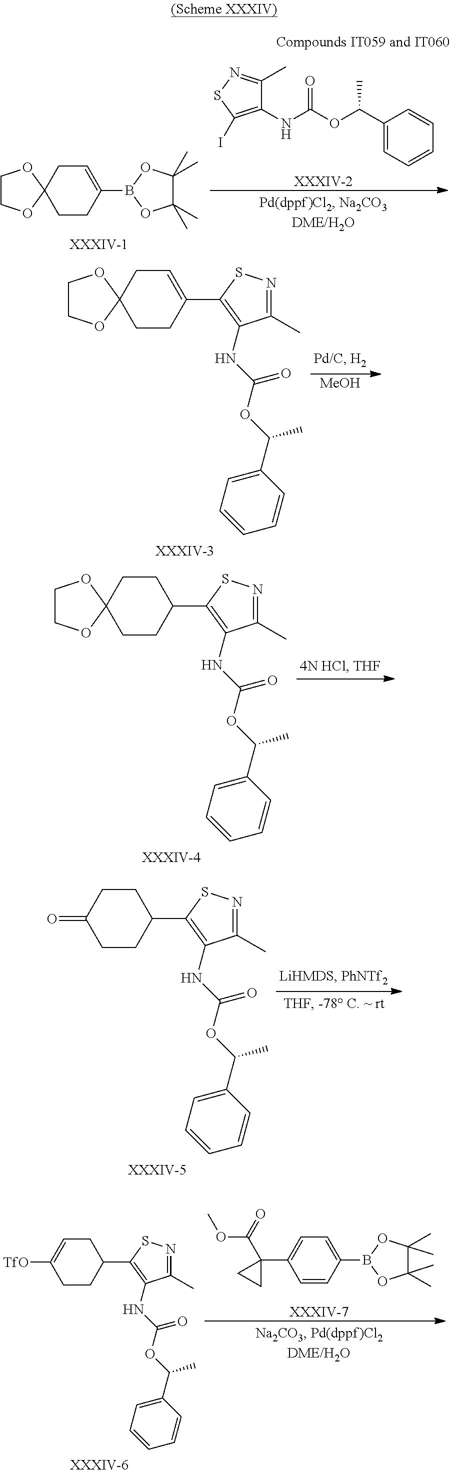

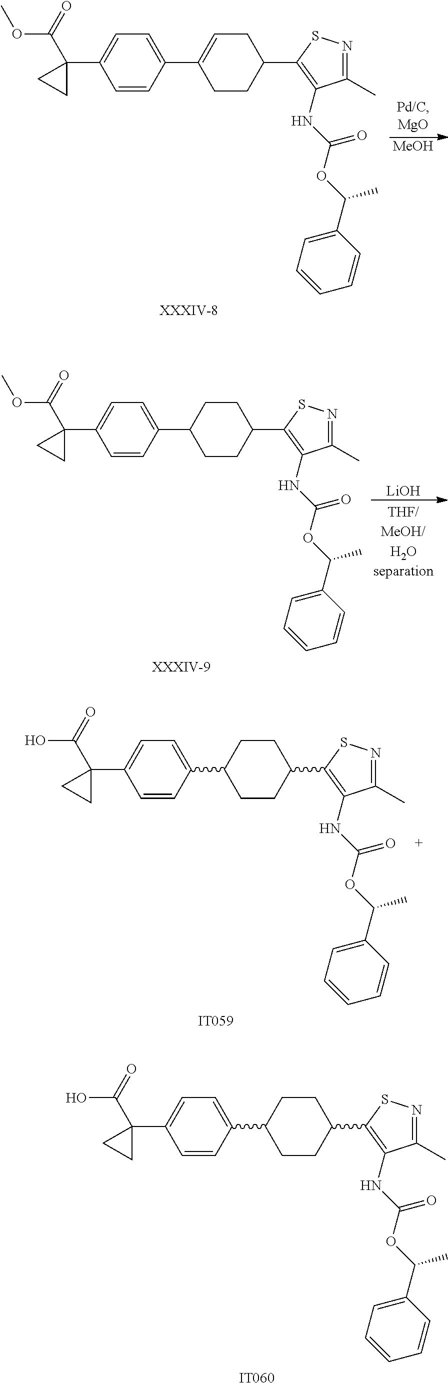

Argon gas was bubbled through a mixture of XXXIV-1 (2.0 g, 7.52 mmol) and XXXIV-2 (2.92 g, 7.52 mmol) in 30 mL of DME/H₂O (v/v=3/1). The Na₂CO₃ (2.39 g, 22.56 mmol) and Pd(dppf)Cl₂ (275 mg, 0.38 mmol) was added. The mixture was heated to 80° C. and stirred overnight. After cooled, the mixture was filtered through Celite and the filtrate was washed with brine, dried over MgSO₄ and concentrated. The residue was purified by flash column chromatography over silica gel (PE:EA=2/1) to afford XXXIV-3 (2.3 g, yield 77%).

A mixture of XXXIV-3 (2 g, 4.99 mmol) and 3 g of Pd/C (w %=10%) in 100 mL of methanol was hydrogenated under hydrogen atmosphere (40 psi) for 20 hours at rt. The mixture was filtered through Celite and the filtrate was concentrated in vacuum to afford XXXIV-4 (1.7 g, yield 85%).

4N aqueous HCl solution (17 mL, 68 mmol) was added slowly to a solution of XXXIV-4 (1.7 g, 4.23 mmol) in 34 mL of THF at 0° C. The mixture was stirred for 5 hs at rt. The mixture was diluted with H₂O, extracted with EA. The combined organic layer was washed with saturated NaHCO₃ solution, brine, dried over MgSO₄ and concentrated. The residue was purified by flash column chromatography over silica gel (PE:EA=5/1) to afford XXXIV-5 (1.2 g, yield 80%).

To a stirred solution of XXXIV-5 (800 mg, 2.23 mmoL) in dry THF (10 mL) was added LiHMDS (1.0N solution in THF, 11.2 mmol) dropwise at −78° C. After addition, the reaction temperature was allowed to rise to rt slowly and the mixture was stirred for 1 h at rt. Then the mixture was re-cooled to −78° C. and a solution of PhNTf₂ (1.6 g, 4.46 mmol) in 2 mL of THF was added slowly. After addition, the reaction temperature was allowed to rise to rt slowly and the mixture was stirred overnight at rt. The reaction mixture was quenched with saturated NH₄Cl aqueous solution, extracted with EtOAc. The combined organic layer was washed with brine, dried and concentrated. The residue was purified by flash column chromatography over silica gel (PE:EA=7/1) to afford XXXIV-6 (300 mg, yield 27.3%).

XXXIV-8 was prepared by reacting XXXIV-6 (120 mg, 0.24 mmol) with XXXIV-7 (148 mg, 0.49 mmol) using the same reaction for the preparation of XXXIV-3 as colourless oil.

A mixture of XXXIV-8 (140 mg, 0.27 mmol), MgO (22 mg, 0.54 mmol) and 210 mg of Pd/C (w %=10%) in 10 mL of MeOH was stirred for 5 h under hydrogen atmosphere at rt. The insoluble substance was filtered off and the filtrate was concentrated in vacuum to afford XXXIV-9 (115 mg, yield 82%) as white solid.

IT059 and IT060 were obtained from LiOH hydrolysis of XXXIV-9 followed by separation. Sodium salt IT059a: ¹H NMR (Methanol-d₄, 400 MHz) δ 7.45-7.46 (m, 2H), 7.39-7.41 (m, 2H), 7.29-7.33 (m, 3H), 7.12 (d, J=8.0 Hz, 2H), 5.82 (q, J=6.4 Hz, 1H), 2.88-2.91 (m, 1H), 2.53-2.55 (m, 1H), 2.27 (s, 3H), 2.05-2.11 (m, 2H), 1.88-1.91 (m, 2H), 1.49-1.62 (m, 7H), 1.41-1.42 (m, 2H), 0.92-0.93 (m, 2H). MS (ESI) m/z (M+H)⁺ 505.2.

Sodium salt IT060: ¹H NMR (Methanol-d₄, 400 MHz): δ 7.37-7.43 (m, 4H), 7.28-7.30 (m, 3H), 7.16 (d, J=8.0 Hz, 2H), 5.80 (q, J=6.4 Hz, 1H), 3.41 (br, 1H), 2.68 (br, 1H), 2.27 (s, 3H), 1.78-1.90 (m, 8H), 1.60 (d, J=6.4 Hz, 3H), 1.46 (br, 2H), 1.00 (br, 2H). MS (ESI) m/z (M+Na)⁺505.02.

### Example 27

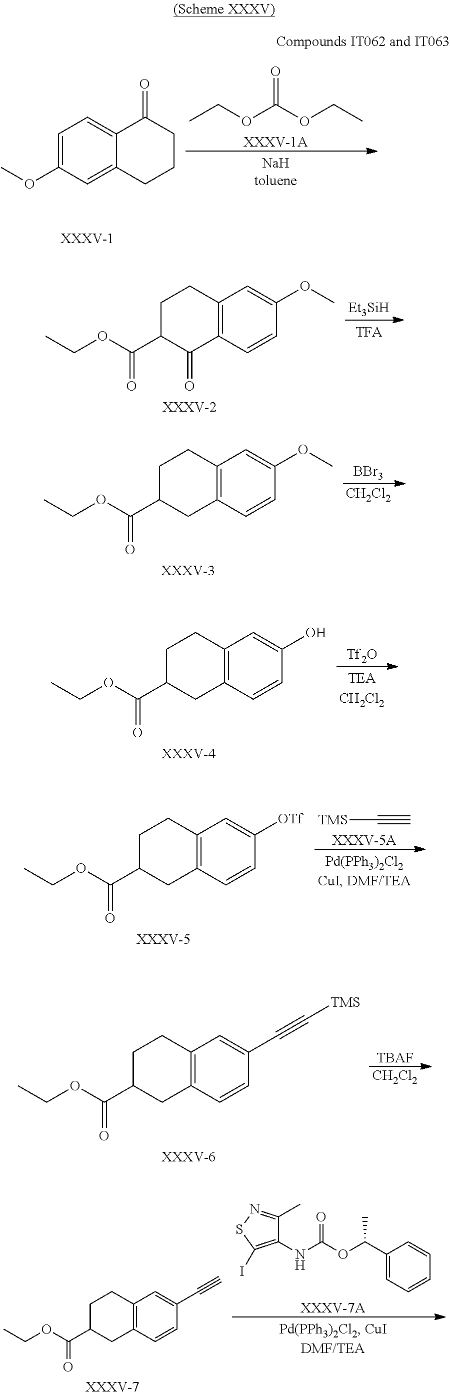

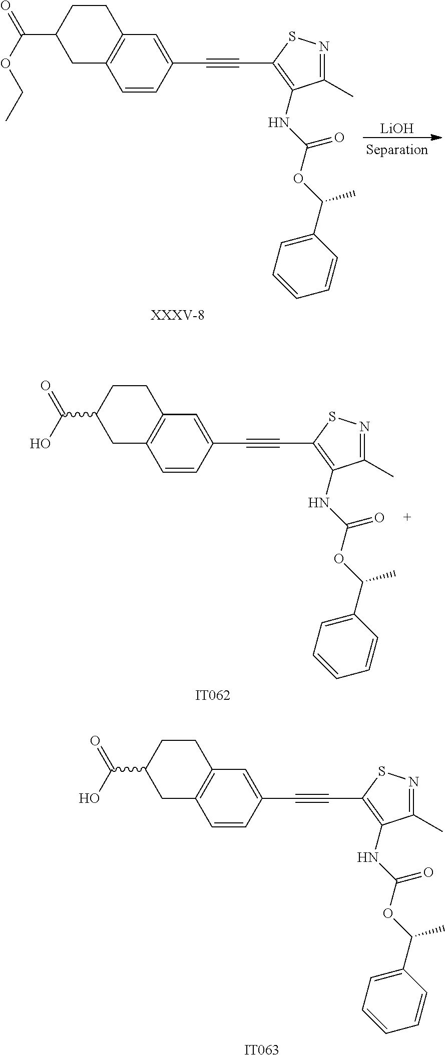

To a solution of XXXV-1A (7.4 g, 62.7 mmol) in toluene (100 mL) was added portion wise NaH (3.7 g, 92.5 mmol) at 25° C. and the mixture was heated at 120° C. for 30 min. Then to the mixture was added a solution of XXXV-1 (5.1 g, 18.5 mmol) in toluene (50 mL). The resulting mixture was stirred at 120° C. for 12 h. After being cooled to rt, aq. HCl (1M, 20 mL) was added to the mixture, and the mixture was extracted with EtOAc. The organics were combined, dried with Na₂SO₄, and concentrated to afford crude XXXV-2 (5.0 g, yield: 78.1%), which was used to next step directly.

To a solution of XXXV-2 (5 g, 20.16 mmol) in TFA (50 mL) was added Et₃SiH (9.5 mL) dropwise, and the resulting mixture was stirred at 25° C. for 12 h. Removed the solvent in vacuo gave an oily residue, which was washed with H₂O, extracted with EtOAc, washed with saturated NaHCO₃. The organics were combined, dried with Na₂SO₄, filtered and concentrated. The residue was purified by column chromatography (PE/EA=30/1) to give XXXV-3 (4 g, yield: 85%).

To a solution of XXXV-3 (1.5 g, 6.4 mmol) in CH₂Cl₂ (30 mL) was added BBr₃ (3.2 g, 12.8 mmol) at −68° C. dropwise. After addition, the mixture was stirred at 25° C. for 2 h. The reaction was poured into ice-water, extracted with CH₂Cl₂. The organic layer was washed with brine, dried over Na₂SO₄, filtered and concentrated. The residue was purified by column chromatography (PE/EA=3/1) to give XXXV-4 (325 mg, yield: 23%).

To a stirred solution of XXXV-4 (625 mg, 2.84 mmol) and TEA (573 mg, 5.68 mmol) in CH₂Cl₂ (20 mL) was added Tf₂O (941 mg, 3.4 mmol) dropwise at −40° C. The mixture was stirred at 18° C. for 2 h. Then H₂O (20 mL) was added, the organic layer were separated, dried with Na₂SO₄, and concentrated to afford crude XXXV-5 (960 mg, yield: 96%), which was used to next step directly.

XXXV-6, XXXV-7 and XXXV-8 were prepared following the similar procedure described in the synthesis of XVII-2, XVII-3, and XVII-5.

IT062 and IT063 were obtained from LiOH hydrolysis of XXXV-8 followed by SFC separation. MS (ESI) m/z (M+H)⁺ 461.1.

Sodium salt IT062a: ¹HNMR (DMSO-d₆, 400 MHz) δ 9.59 (brs, NH), 7.29-7.39 (m, 5H), 7.11-7.15 (m, 3H), 5.77-5.82 (m, 1H), 2.67-2.85 (m, 4H), 2.28 (s, 3H), 2.21-2.23 (m, 1H), 1.98-2.01 (m, 1H), 1.61-1.64 (m, 1H), 1.53 (d, J=6.0 Hz, 3H). MS (ESI) m/z (M+H)⁺ 461.1.

Sodium salt IT063a: ¹HNMR (DMSO-d₆, 400 MHz) δ 9.56 (brs, 1H), 7.31-7.39 (m, 5H), 7.12-7.16 (m, 3H), 5.77-5.82 (q, 1H), 2.65-2.87 (m, 4H), 2.36-2.37 (m, 1H), 2.28 (s, 3H), 2.01-2.04 (m, 1H), 1.64-1.65 (m, 1H), 1.52-1.53 (d, J=6.0 Hz, 3H). MS (ESI) m/z (M+H)⁺ 461.1.

### Example 28

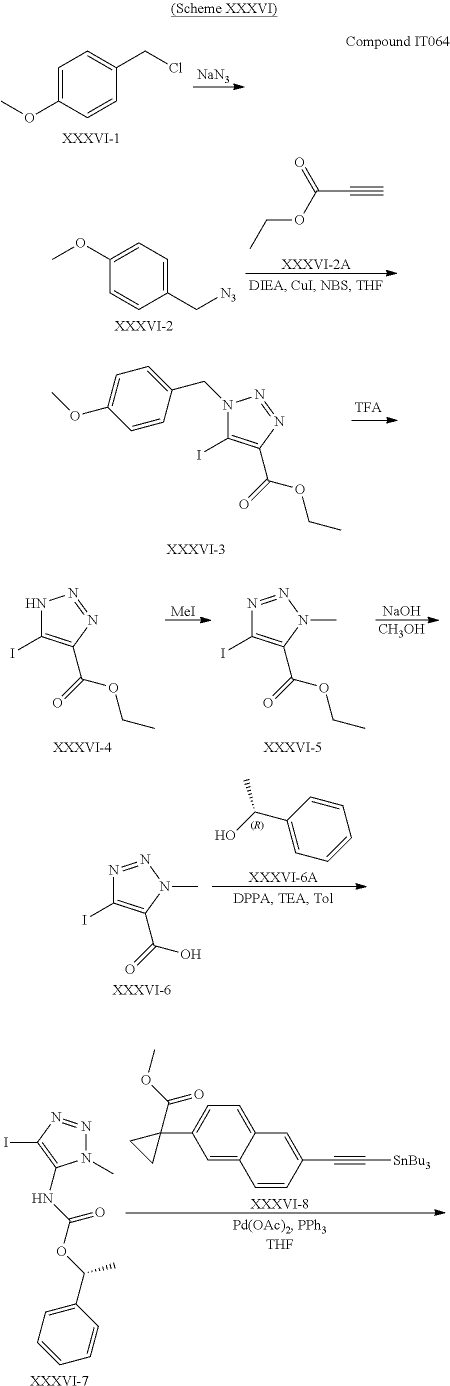

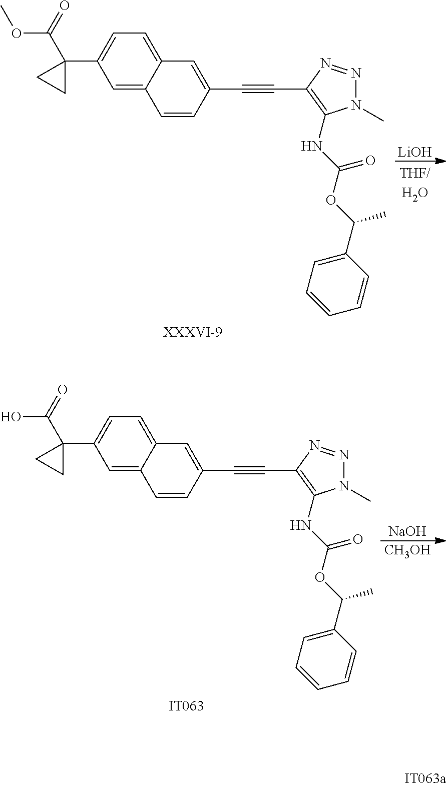

To a solution of XXXVI-1 (20 g, 0.127 mol) in DMF (150 mL) was added NaN₃ (8.2 g, 0.127 mol). After addition, the mixture was stirred for 24 h at 25° C. The reaction mixture was extracted with MTBE. The combined organic phase was washed with brine, dried over Na₂SO₄, filtered and concentrated to give crude XXXVI-2 (20.8 g, crude yield: 100%), which was used to next step directly.

To a solution of XXXVI-2 (20.8 g, 0.127 mol) in THF (200 mL) was added ethyl propiolate XXXVI-2A (12.5 g, 0.127 mol), CuI (24.2 g, 0.127 mol), DIEA (16.4 g, 0.127 mol) and NBS (25 g, 0.25 mol). The reaction mixture was flushed with nitrogen and stirred for 3 h. Water was added and extracted with EtOAc. The organic layer was combined, dried over Na₂SO₄, and concentrated. The residue was purified by column chromatography (PE:EA=5:1) to give XXXVI-3 (20 g, yield: 40.8%).

A mixture of XXXVI-3 (20 g, 51.7 mmol) in TFA (200 mL) was stirred at 65° C. for 3 h. The reaction mixture was concentrated, and the residue was purified by column chromatography (PE:EA=5:1) to give XXXVI-4 (12 g, yield: 87.6%).

To a solution of XXXVI-4 (12 g, 45 mmol) in CH₃CN (100 mL) was added MeI (12.7 g, 90 mmol), K₂CO₃ (12.4 g, 90 mmol). The reaction mixture was stirred for 3 hs at 25° C. The mixture was diluted with EtOAc, washed with brine, dried over Na₂SO₄, filtered and concentrated. The residue was purified and separated by prep-HPLC to give XXXVI-5 (2.1 g, yield: 13.3%). The structure was confirmed by HMBC.

XXXVI-6 was prepared from XXXVI-5 following the similar procedure described in the synthesis of XII-4 using NaOH in place of LiOH.

XXXVI-7 was prepared from reacting XXXVI-6 with XXXVI-6A following the similar procedure described in the synthesis of XII-5.

XXXVI-9 was prepared from reacting XXXVI-7 with XXXVI-8 following the similar procedure described in the synthesis of XII-8.

IT064 and sodium salt IT064a were prepared following the similar procedure described in the synthesis of IT001 and IT001a. IT064a: ¹H NMR (DMSO-d₆ 400 MHz) δ 7.95 (s, 1H), 7.71-7.82 (m, 3H), 7.68 (s, 1H), −7.92 (m, 3H), 7.42 (d, J=7.6 Hz, 1H), 7.33-7.37 (m, 6H), 5.77-5.81 (q, 1H), 3.78 (s, 3H), 1.51 (d, J=6.4 Hz, 3H), 1.28 (d, J=2.4 Hz, 2H), 0.85 (br, 2H). MS (ESI) m/z (M+H)⁺ 481.1.

IT069 was prepared following the general synthetic scheme of IT064 replacing XXXVI-8 with

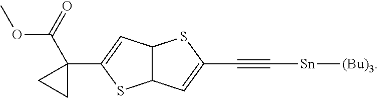

MS (ESI) m/z (M+H)⁺ 493.0. Sodium salt IT069a: ¹H NMR (DMSO-d₆ 400 MHz): δ 7.7.34-7.40 (m, 2H), 7.25-7.34 (m, 4H), 7.02 (s, 1H), 5.82-5.87 (m, 1H), 3.87 (s, 3H), 1.58-1.59 (m, 5H), 1.17 (br, 2H). MS (ESI) m/z (M+H)⁺ 493.0.

### Example 29

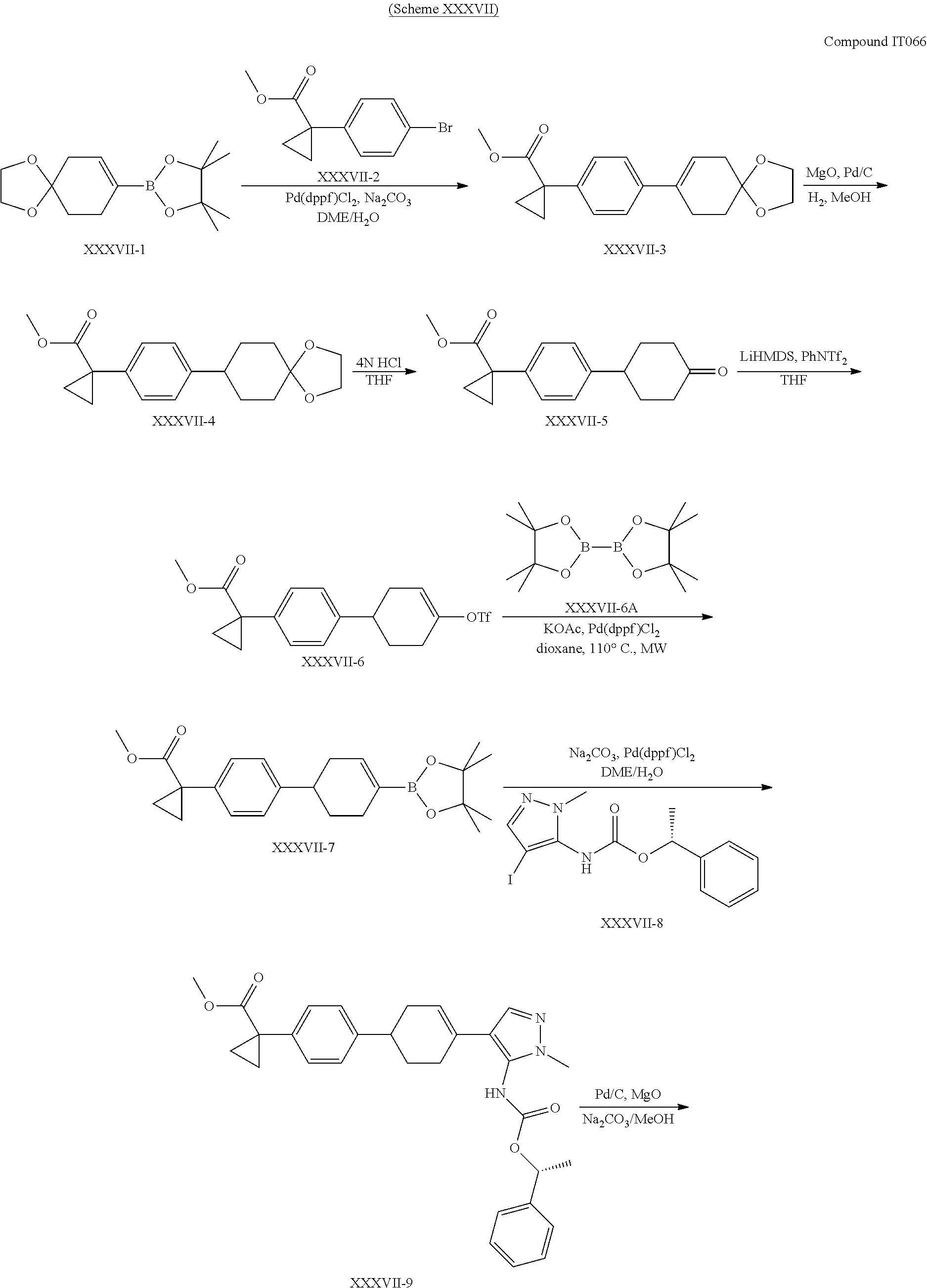

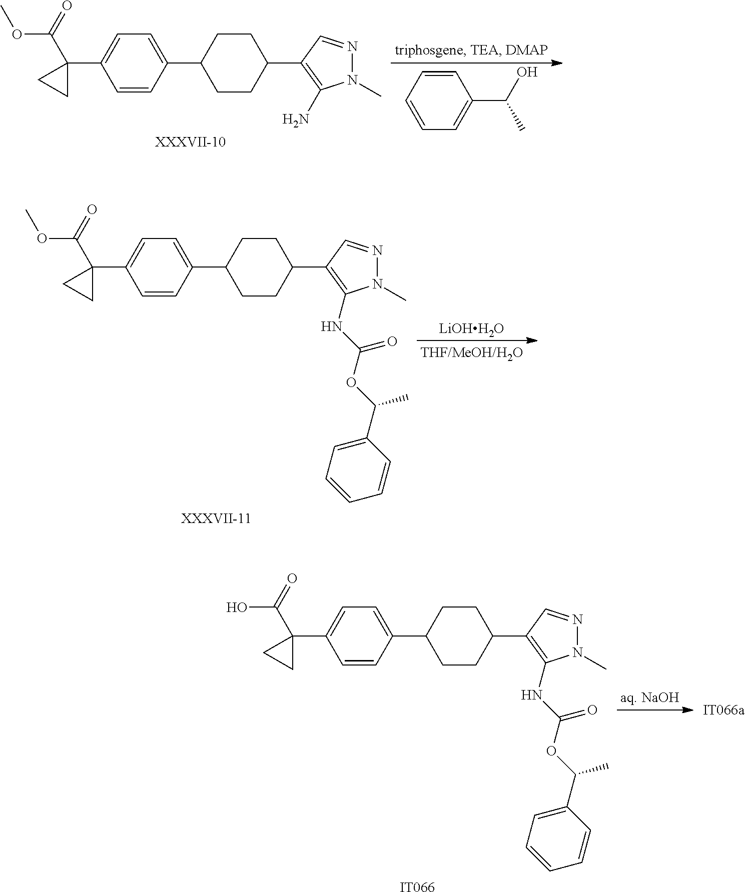

XXXVII-3 was prepared by reacting XXXVII-1 with XXXVII-2 following the similar procedure described in the synthesis of III-5.

A mixture of XXXVII-3 (2.86 g, 9.10 mmol) and 430 mg of Pd/C (w %=5%) in 100 mL of methanol was hydrogenated under hydrogen atmosphere (35 psi) for 20 hours. The mixture was filtered through Celite and the filtrate was concentrated in vacuum to afford XXXVII-4 (2.7 g, yield 94%).

4N aqueous HCl solution (20 mL, 80 mmol) was added slowly to a solution of XXXVII-4 (2.7 g, 8.53 mmol) in 40 mL of THF at 0° C. The mixture was stirred for 2 hs at rt. The mixture was diluted with H₂O, extracted with EtOAc. The combined organic layer was washed with saturated NaHCO₃ solution, brine, dried over MgSO₄ and concentrated. The residue was purified by flash column chromatography over silica gel (PE:EA=5/1) to afford XXXVII-5 (2.3 g, yield 99%).

XXXVII-6 was prepared from XXXVII-5 following the similar procedure described in the synthesis of XXXIV-6. XXXVII-7 was prepared from reacting XXXVII-6 with XXXVII-6A following the similar procedure described in the synthesis of III-3.

XXXVII-9 was prepared following the similar procedure described in the synthesis of III-5.

A mixture of XXXVII-9 (110 mg, 0.22 mmol), MgO (18 mg, 0.44 mmol), Na₂CO₃ (46 mg, 0.44 mmol) and 22 mg of Pd/C (w %=5%) in 10 mL of MeOH was hydrogenated under hydrogen atmosphere (35 psi) at rt. The insoluble substance was filtered off and the filtrate was concentrated. The residue was treated with EtOAc and H₂O. The organic layer was separated and the aqueous layer was extracted with EtOAc. The combined organic layer was washed with brine, dried and concentrated to afford XXXVII-10 (50 mg, yield 64%).

Triphosgene (84 mg, 0.28 mmol) was added to a solution of XXXVII-10 (100 mg, 0.28 mmol), TEA (143 mg, 1.41 mmol) and DMAP (35 mg, 0.28 mmol) in 5 mL of dry dichloromethane at 5° C. Then (R)-1-phenylethanol (172 mg, 1.41 mmol) was added. The mixture was stirred overnight at rt. The mixture was diluted with dichloromethane, washed with H₂O, saturated NaHCO₃ aqueous solution, brine, dried and concentrated to afford XXXVII-11 (150 mg, crude), which was used directly without further purification.

IT066 and sodium salt IT066a were prepared following the similar procedure described in the synthesis of IT001 and IT001a. IT066: MS (ESI) m/z (M+H)⁺ 488.1. IT066a: ¹HNMR (400 MHz, Methanol-d₄) δ 7.25-7.44 (m, 8H), 7.09-7.13 (m, 2H), 5.79-5.87 (m, 1H), 3.64 (s, 3H), 2.63-2.87 (m, 1H), 2.50-2.57 (m, 1H), 1.61-1.92 (m, 6H), 1.49-1.54 (m, 5H), 1.40-1.41 (m, 2H), 0.91-0.93 (m, 2H). MS (ESI) m/z (M+H)⁺ 488.2.

### Example 30

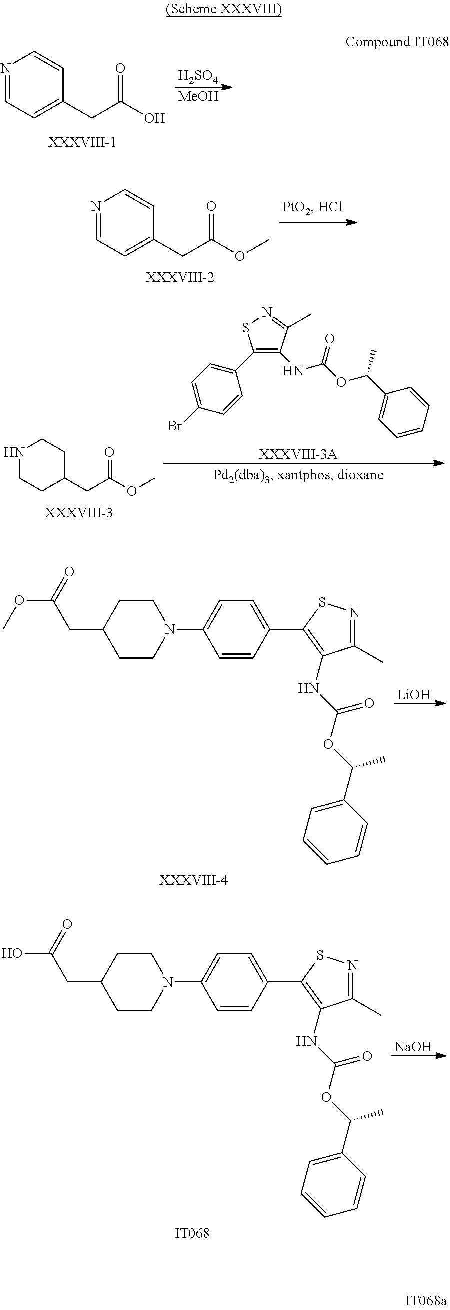

To a solution of XXXVIII-1 (4 g, 29.2 mmol) in MeOH (40 mL) was dropwise H₂SO₄ (1 g). Then the mixture was heated to reflux for about 2 hs. Then the MeOH was evaporated in vacuo. Water was added and extracted with EtOAc. The combined organic layers were washed with brine, dried over Na₂SO₄ and concentrated under vacuo. The crude product was purified by column chromatography (PE:EA=10/1) to afford XXXVIII-2 (3.5 g, yield: 79.55%).

To a solution of XXXVIII-2 (2 g, 13.25 mmol) in MeOH (20 mL) was added PtO₂ (200 mg) and HCl (6N, 2 mL) under H₂ atmosphere (30Psi) at rt. Then the mixture was stirred at this atmosphere for about 2 hs. Then the solution was filtered and the liquid was concentrated. The crude XXXVIII-3 (1.8 g, yield: 86.5%) was used to next step directly.

To a solution of XXXVIII-3 (136 mg, 0.866 mmol) in dioxane (4 mL) was added compound XXXVIII-3A (300 mg, 0.72 mmol) and Xantphos (117 mg, 0.17 mmol) and Cs₂CO₃ (468 mg, 1.732 mmol) and Pd₂(dba)₃ (119 mg, 0.17 mmol) under N₂ atmosphere. Then the mixture was heated to reflux and stirred for 4 hs. Then dioxane was removed under vacuo, water (2 mL) was added and extracted with EtOAc. The combined organic layers were washed with brine, dried over Na₂SO₄ and concentrated under vacuo. The residue was purified by column chromatography (PE:EA=5/1) to afford XXXVIII-4 (120 mg, yield: 28.98%).

IT068 and sodium salt IT068a were prepared following the similar procedure described in the synthesis of IT001 and IT001a. IT068: MS (ESI) m/z (M+H)⁺ 480.1. IT068a: ¹H NMR (DMSO-d₆, 400 MHz) δ 8.69 (s, 1H), 7.27-7.32 (m, 7H), 6.86-6.88 (m, 2H), 5.68-5.73 (m, 1H), 3.71-3.73 (m, 2H), 2.73-2.79 (m, 2H), 2.18 (s, 3H), 2.04 (br, 2H), 1.74-1.85 (m, 1H), 1.45 (br, 3H), 1.23-1.26 (m, 2H). MS (ESI) m/z (M+H)⁺ 480.1.

### Example 31

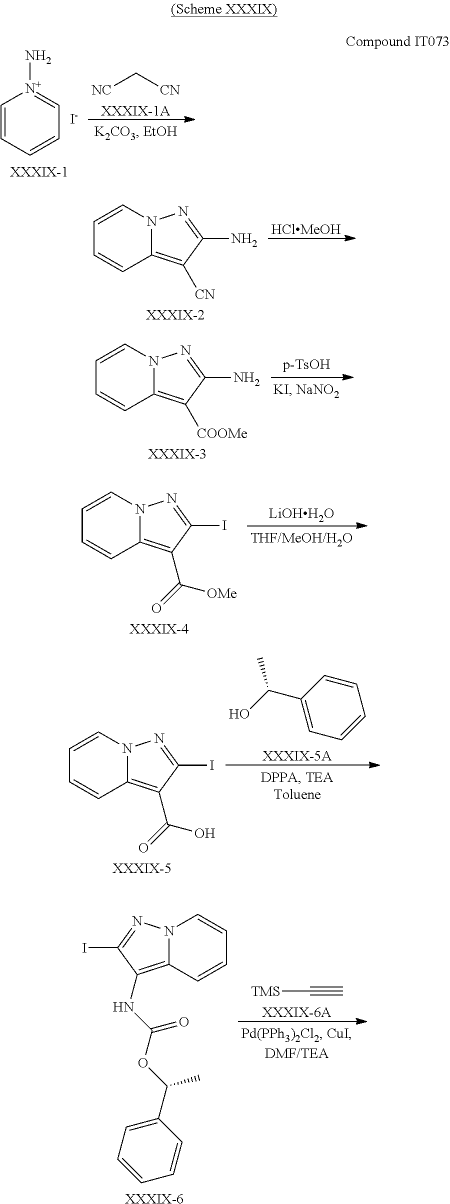

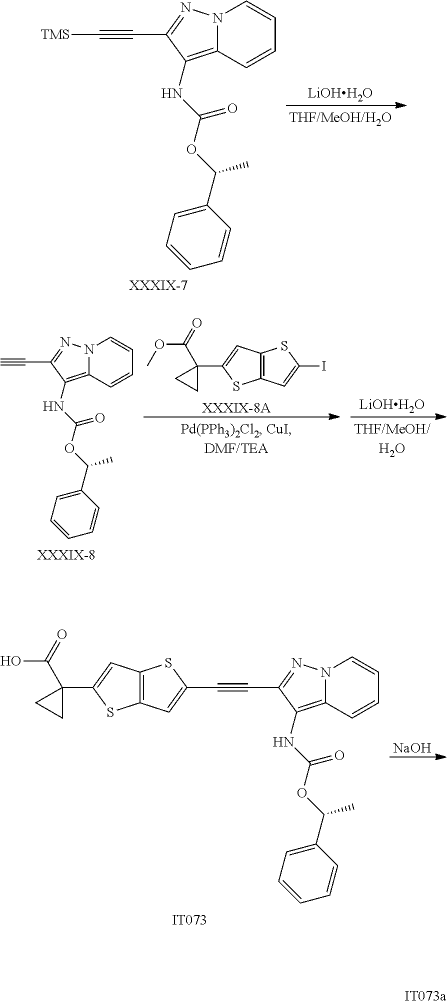

To a solution of XXXIX-1 (10 g, 45 mmol) in EtOH (150 mL) was added XXXIX-1A (2.85 mL, 45 mmol) and K₂CO₃ (12.4 g, 90 mmol). The mixture was stirred at 90° C. for 24 h. After concentrated, the mixture was diluted with EtOAc, washed with brine, dried over Na₂SO₄, filtered and concentrated as XXXIX-2 (3 g, yield 42%).

A mixture of XXXIX-2 (1.1 g, 6.96 mmol) in HCl/MeOH (4N, 20 mL) was stirred at 80° C. for 24 h. After concentrated, the mixture was diluted with EtOAc, washed with brine, dried over Na₂SO₄, filtered and concentrated. The residue was purified by column (PE/EA=2/1) to afford XXXIX-3 (600 mg, yield 45.1%).

To a solution of p-TsOH.H₂O (1.79 g, 9.42 mmol) in MeCN (10 mL) was added XXXIX-3 (600 mg, 3.14 mmol). Then a solution of NaNO₂ (433 mg, 6.28 mmol) and KI (1.29 g, 7.85 mmol) in H₂O (2 mL) was gradually added. The reaction mixture was stirred for 3 h. Then the reaction mixture was then added H₂O, NaHCO₃ and Na₂S₂O₃. The precipitated aromatic iodide was filtered and by flash chromatography on silica gel (PE/EA=5/1) to afford XXXIX-4 (520 mg, yield 54.8%).

To a stirred solution of XXXIX-4 (600 mg, 2 mmol) in THF/MeOH/H₂O=1/1/1 (6 mL) was added LiOH.H₂O (420 mg, 10 mmol). After the addition, the solution was stirred overnight at rt. The solution was concentrated in vacuo, the aqueous layer was adjust pH to 2 with 1N HCl, and extracted with EtOAc. The organic layer was separated, dried and concentrated to afford crude XXXIX-5 (530 mg, crude), which was used to next step directly.

XXXIX-6, XXXIX-7, XXXIX-8, IT073 and its sodium salt IT073a were prepared following the similar procedure described in the synthesis of XII-5 and the alternative synthetic scheme XIII of IT017. IT073: MS (ESI) m/z (M+H) 527.9. IT073a: ¹H NMR (400 MHz, Methanol-d₄): δ 8.42 (d, J=6.4 Hz, 1H), 7.23-7.47 (m, 8H), 7.07 (s, 1H), 6.94-6.97 (m, 1H), 5.87-5.89 (m, 1H), 1.62-1.65 (m, 5H), 1.21-1.23 (m, 2H). MS (ESI) m/z (M+H)⁺ 528.0.

### Example 32

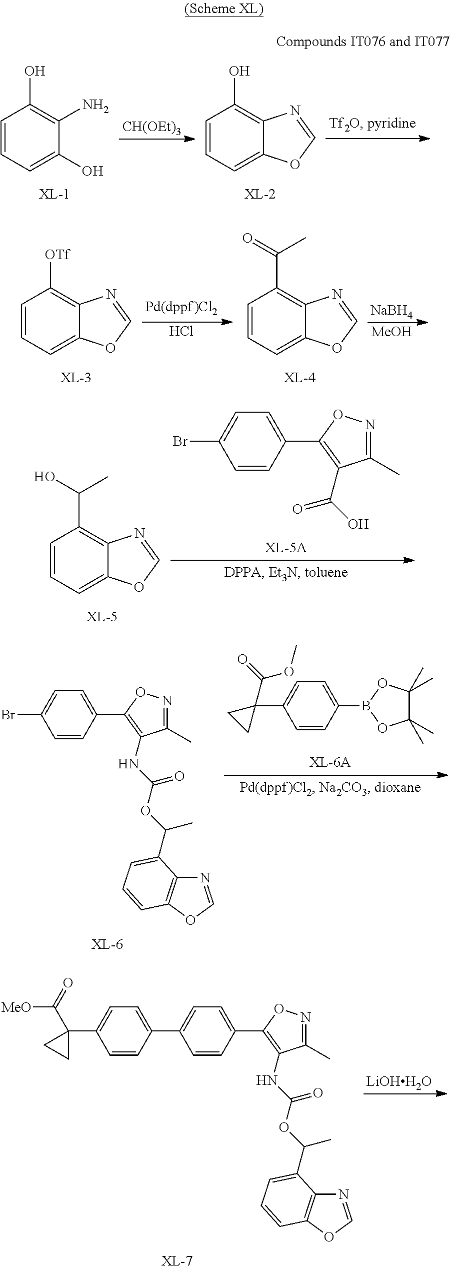

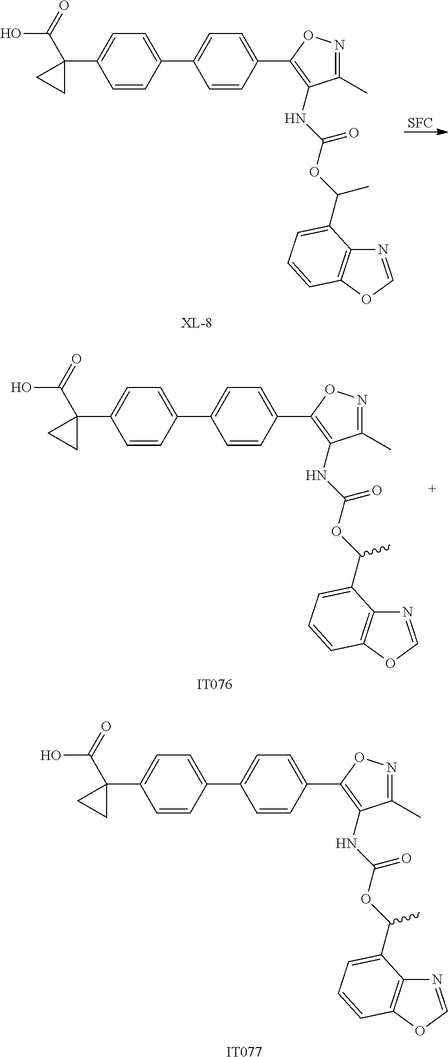

The solution of XL-1 (1 g, 8 mmol) in triethyl orthoformate (10 mL) was stirred at 130° C. for 2 hrs. Then the excess triethyl orthoformate was removed by evaporation. The residue was purified by column over silica gel (PE:EA=10/1) to afford XL-2 (0.68 g, yield 62%).

To a solution of XL-2 (500 mg, 3.7 mmol) in DCM (10 mL) was added trifluoromethanesulfonic anhydride (1.6 g, 5.6 mmol) and pyridine (585 mg, 7.4 mmol) at 0° C. The mixture was stirred at rt for 5 hrs. The mixture was diluted with water and extracted with EA. The organic layer was dried over Na₂SO₄, concentrated and purified by column over silica gel (PE:EA=10/1) to provide XL-3 (450 mg, yield: 46%).

A mixture of XL-3 (1.5 g, 5.6 mmol), tributyl (1-ethoxyvinyl)tin (2.3 g, 6.2 mmol), LiCl (24 mg, 0.56 mmol) and Pd(dppf)Cl₂ (0.3 g, 0.28 mmol) in dioxane (25 mL) was stirred at 100° C. for 4 hrs. The mixture was cooled to rt, then HCl (30 mL, 3N) and DCM (30 mL) was added. After stirred for 30 mins, the organic layer was separated, dried over Na₂SO₄, concentrated and purified by column over silica gel (PE:EA=3/1) to provide XL-4 (600 mg, yield 67%).

To a solution of XL-4 (200 mg, 1.24 mmol) in 5 mL of MeOH/H₂O (v/v=5/1) was added NaBH₄ (94 mg, 2.48 mmol) at 0° C. Then the mixture was stirred at 0° C. for 30 mins. Then NH₄Cl (aq, 2 mL) was added and most of MeOH was evaporated and the mixture was extracted with DCM. Then 5 mL of toluene was added and the volatile solvent DCM was concentrated at rt to afford XL-5 (1.24 mmol) which was used for next step directly.

To a solution of XL-5 (200 mg, 1.23 mmol) in toluene (10 mL) was added XL-5A (413 mg, 1.47 mmol), DPPA (404 mg, 1.47 mmol) and Et₃N (248 mg, 2.46 mmol) under nitrogen atmosphere. Then the mixture was heated to reflux for 2 hrs. Then most of toluene was evaporated. The residue was diluted with 3 mL of water and extracted with EA. The combined organic layers were washed with brine, dried over Na₂SO₄ and concentrated under vacuo. The crude product was purified by silica gel (PE:EA=1/1) to afford XL-6 (150 mg, yield 27.6%).

XL-7 and XL-8 were prepared following the similar procedure described in the synthesis of IT031. Enantiomers IT076 and IT077 were obtained from SFC separation of XL-8. IT076: ¹H NMR (Methanol-d₄, 400 MHz): δ 8.49 (s, 1H), 7.45-7.81 (m, 11H), 6.37 (br, 1H), 2.19 (s, 3H), 1.74 (d, J=6.0 Hz, 3H), 1.49 (br, 2H), 1.02 (br, 2H). MS (ESI) m/z (M+H)⁺ 524.2. IT077: ¹H NMR (Methanol-d₄, 400 MHz): δ 8.49 (s, 1H), 7.44-7.82 (m, 11H), 6.39 (br, 1H), 2.19 (s, 3H), 1.74 (d, J=6.0 Hz, 3H), 1.46 (br, 2H), 0.97 (br, 2H). MS (ESI) m/z (M+H)⁺ 524.2.

### Example 33

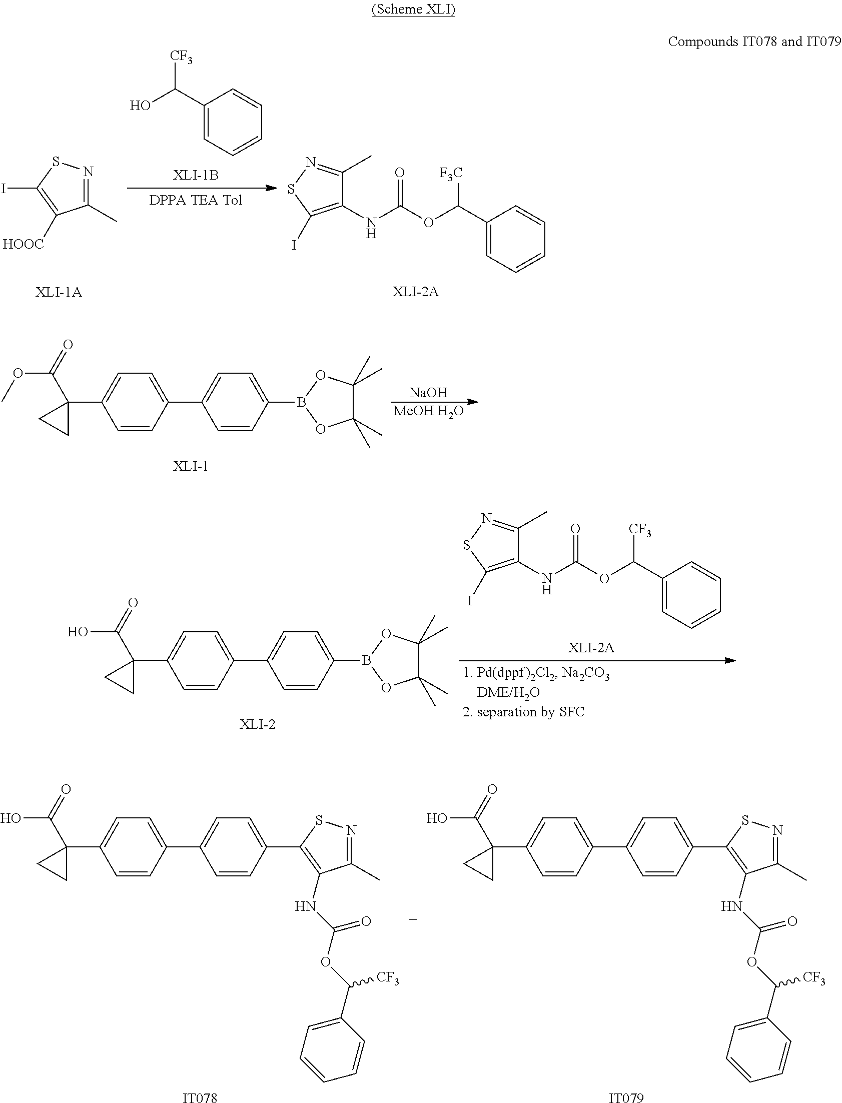

To a solution of XLI-1A (500 mg, 1.86 mmol) in dry toluene (10 mL) was added XLI-1B (393 mg, 2.23 mmol), triethylamine (373 mg, 3.72 mmol) and DPPA (611 mg, 2.23 mmol). The reaction mixture was heated to 80° C. for 3 h. The mixture was diluted with EtOAc, washed with brine, dried over Na₂SO₄, filtered and concentrated. The residue was purified by column (PE/EA=5/1) to give XLI-2A (800 mg, yield: 97%).

Enantiomers IT078 and IT079 were obtained by deprotection of XLI-1 with NaOH and subsequent Suzuki coupling with XLI-2A following the similar procedure described in the synthesis of III-5 followed by SFC separation. IT078: ¹H NMR (Methanol-d₄, 400 MHz): δ 7.51-7.63 (m, 8H), 7.44-7.48 (m, 5H), 6.15-6.20 (m, 1H), 2.33 (s, 1H), 1.61-1.63 (m, 2H), 1.23-1.26 (m, 2H). MS (ESI) m/z (M+H)⁺ 553.1. IT079: ¹H NMR (Methanol-d₄, 400 MHz): δ 7.51-7.62 (m, 8H), 7.44-7.48 (m, 5H), 6.15-6.20 (m, 1H), 2.33 (s, 1H), 1.60-1.63 (m, 2H), 1.23-1.26 (m, 2H). MS (ESI) m/z (M+H)⁺ 553.1.

### Example 34

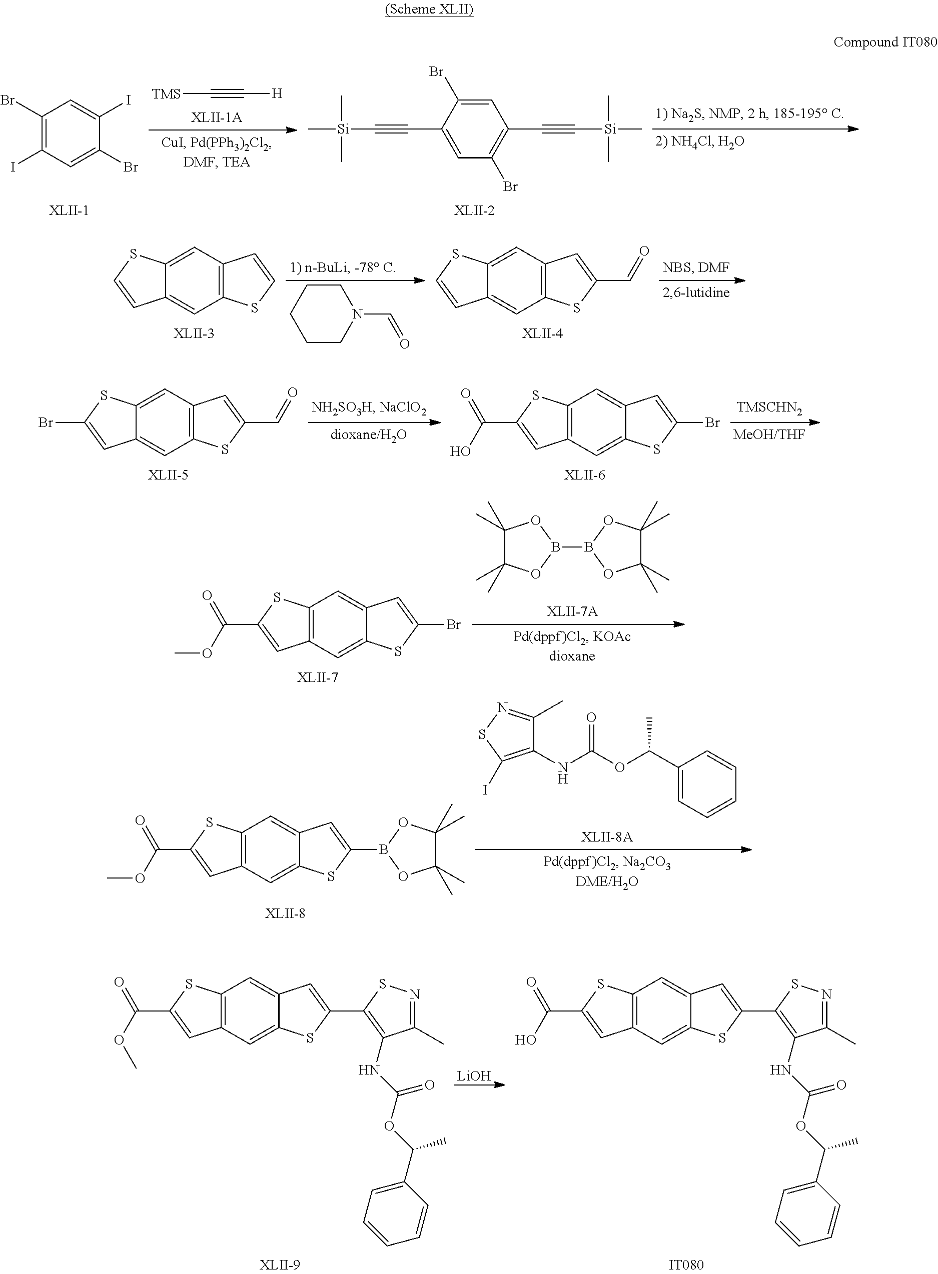

To a solution of XLII-1 (2 g, 4.1 mmol), CuI (78 mg, 0.41 mmol), and Pd(PPh₃)₂Cl₂ (287 mg, 0.41 mmol) in DMF (60 mL) and TEA (20 mL) (DMF was degassed through the solvent by bubbling N₂ for 15 min prior to use) was added XLII-1A (0.8 g, 8.2 mmol) dropwise at 0° C. After addition, the mixture was stirred at 4° C. for 12 h. The mixture was washed with H₂O, extracted with EtOAc. The organics were combined, dried with Na₂SO₄, filtered and concentrated. The residue was purified by column (PE) to afford XLII-2 (1.2 g, yield 68.6%).

To a stirred solution of Na₂S (2.7 g, 11.2 mmol) in NMP (72 mL) was added XLII-2 (1.2 g, 2.8 mmol). The mixture was heated at 185° C. for 2 h. The mixture was quenched with saturated NH₄Cl, extracted with EtOAc. The organics were combined, dried with Na₂SO₄, filtered and concentrated. The residue was purified by column (PE) to give XLII-3 (300 mg, yield 56%).

A solution of n-BuLi (2.5 M in hexane, 2.3 mL, 5.78 mmol) was added dropwise to suspension of XLII-3 (1.0 g, 5.26 mmol) in 25 mL of dry THF at −78° C. The mixture was stirred for 1.5 hours at −78° C. Then a solution of N-carbaldehyde (1.2 mL, 10.51 mmpl) in 2 mL of THF was added slowly. The mixture was stirred at −78° C. for 3 h then the temperature was slowly raise to rt and mixture was stirred overnight. The reaction mixture was quenched by addition of saturated NH₄Cl aqueous solution. The mixture was diluted with H₂O and extracted with EA. The combined organic layer was washed with brine, dried and concentrated. The residue was washed with TBME to afford—XLII-4 (0.9 g, yield 78%), which was used for next step directly.

N-bromosuccinimide (1.4 g, 7.87 mmol) was added in portions to a solution of XLII-4 (800 mg, 3.66 mmol) and 2,6-lutidine (400 mg, 3.73 mmol) in 30 mL of DMF. The mixture was heated to 60° C. and stirred overnight. The mixture was poured into 100 mL of H₂O. The precipitate was collected and dried in vacuum to afford XLII-5 (1.1 g, crude yield 100%) as a yellow solid, which was used for next step directly.

NH₂SO₃H (1.57 g, 14.13 mmol) was added to suspension of XLII-5 (700 mg, 2.36 mmol) in 24 mL of dioxane/H₂O (v/v=7/3). Then NaClO₂ (278 mg, 3.07 mmol) was added. The mixture was stirred for 3 hrs at rt. The mixture was poured in 30 mL of water. The precipitate was collected and purified by prep-HPLC to afford XLII-6 (90 mg, yield 12%).

A solution of (trimethylsilyl)diazomethane in hexane (2 N, 0.17 mL, 0.33 mmol) was added to a suspension of XLII-6 (70 mg, 0.22 mmol) in 1 mL of MeOH and 2 mL of THF. The mixture was stirred overnight at rt. Additional (trimethylsilyl)diazomethane (2 N in hexane, 0.17 mL, 0.33 mmol) was added and the mixture was further stirred for 5 hrs at rt. The mixture was concentrated to afford XLII-7 (70 mg, crude yield), which was used for next step directly.

XLII-8, XLII-9 and IT080 were prepared following the similar procedure described in the preparation of VI-6, VI-7 and IT001. IT080: ¹H NMR (400 MHz, Methanol-d₄): δ 8.57 (s, 1H), 8.28 (s, 1H), 8.16 (s, 1H), 7.84 (s, 1H), 7.17 (br, 5H), 5.67 (q, J=6.4 Hz, 1H), 2.42 (s, 3H), 1.38 (d, J=6.4 Hz, 3H). MS (ESI) m/z (M+H)⁺ 494.9.

IT112 was prepared following the similar procedure described in the synthesis of IT080 using methyl 1-(7-(4-bromophenyl)-2,3-dihydro-1H-inden-4-yl)cyclopropanecarboxylate in place of XLII-7 and (R)-1-phenylethyl (4-iodo-1-methyl-1H-pyrazol-5-yl)carbamate in place of XLII-8A. ¹H NMR (Methonal-d₄, 400 MHz): δ: 7.76 (s, 1H), 7.31-7.46 (br, 8H), 7.12-7.18 (m, 3H), 5.84 (s, 1H) 3.73 (s, 3H), 2.95-3.02 (m, 4H), 2.04-2.09 (m, 2H), 1.63 (br, 5H), 1.21 (br, 2H). MS (ESI) m/z (M+H)⁺ 522.1.

### Example 35

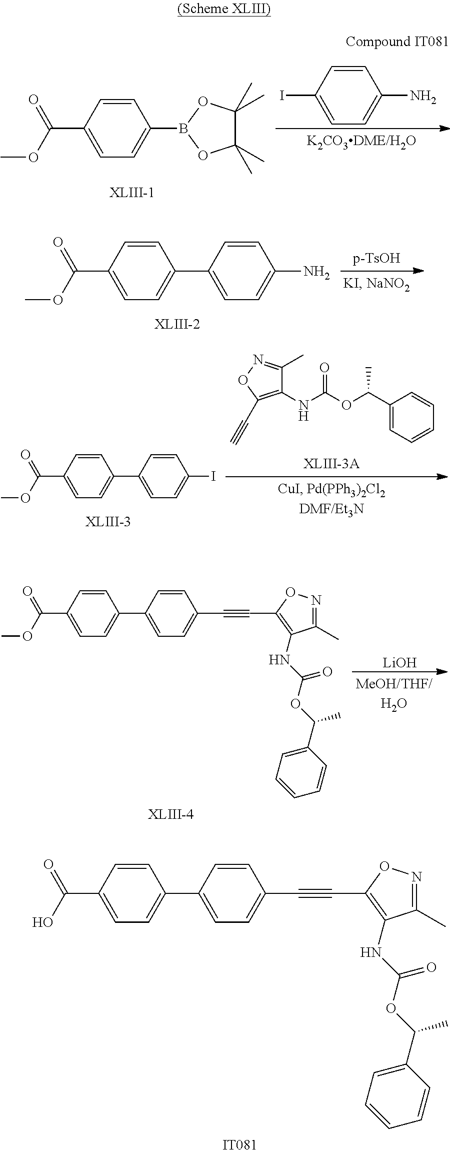

The mixture of XLIII-1 (3 g, 11.44 mmol), 4-iodoaniline (2.76 g, 12.59 mmol), Na₂CO₃ (2.46 g, 22.89 mmol) and Pd(dppf)Cl₂ in DME/H₂O (80 mL, v/v=3/1) was heated to reflux under nitrogen for overnight. After concentrated, the residue was partitioned between H₂O and DCM, and the aqueous phase was extracted with DCM. The combined organic layer was washed with brine, dried over Na₂SO₄, concentrated. The residue was purified by column (PE/EA=5/1) on silica gel to afford XLIII-2 (3 g, yield: 42.3%).

To a solution of p-TsOH.H₂O (2.76 g, 14.5 mmol) in MeCN (60 mL) was added XLIII-2. The resulting suspension of XLIII-2 (1.1 g, 4.84 mmol) salt was cooled to 10-15° C. and to the mixture was added, gradually a solution of NaNO₂ (0.84 g, 12.1 mmol) and KI (1.6 g, 9.69 mmol) in H₂O. The reaction mixture was stirred for 10 min then allowed to come 20° C. and stirred for 3 hrs. The reaction mixture was quenched with H₂O, NaHCO₃ and Na₂S₂O₃. The precipitated aromatic iodide was filtered and by flash chromatography (PE/EA=10/1) to afford XLIII-3 (500 mg, yield: 31.25%).

XLIII-4 and IT081 were prepared following the similar procedure described in the preparation of I-6 and IT001. IT081: MS (ESI) m/z (M+H)⁺ 466.9. Sodium salt IT081a: ¹H NMR (DMSO-d₆, 400 MHz): δ 9.56 (s, 1H), 7.96 (d, J=7.6 Hz, 2H), 7.78 (d, J=8.4 Hz, 2H), 7.66 (d, J=7.6 Hz, 2H), 7.55 (d, J=8.0 Hz, 2H), 7.32-7.38 (m, 5H), 5.77 (q, J=6.0 Hz, 1H), 2.16 (s, 3H), 1.51-1.53 (d, J=6.0 Hz, 3H). MS (ESI) m/z (M+H)⁺ 466.9.

### Example 36

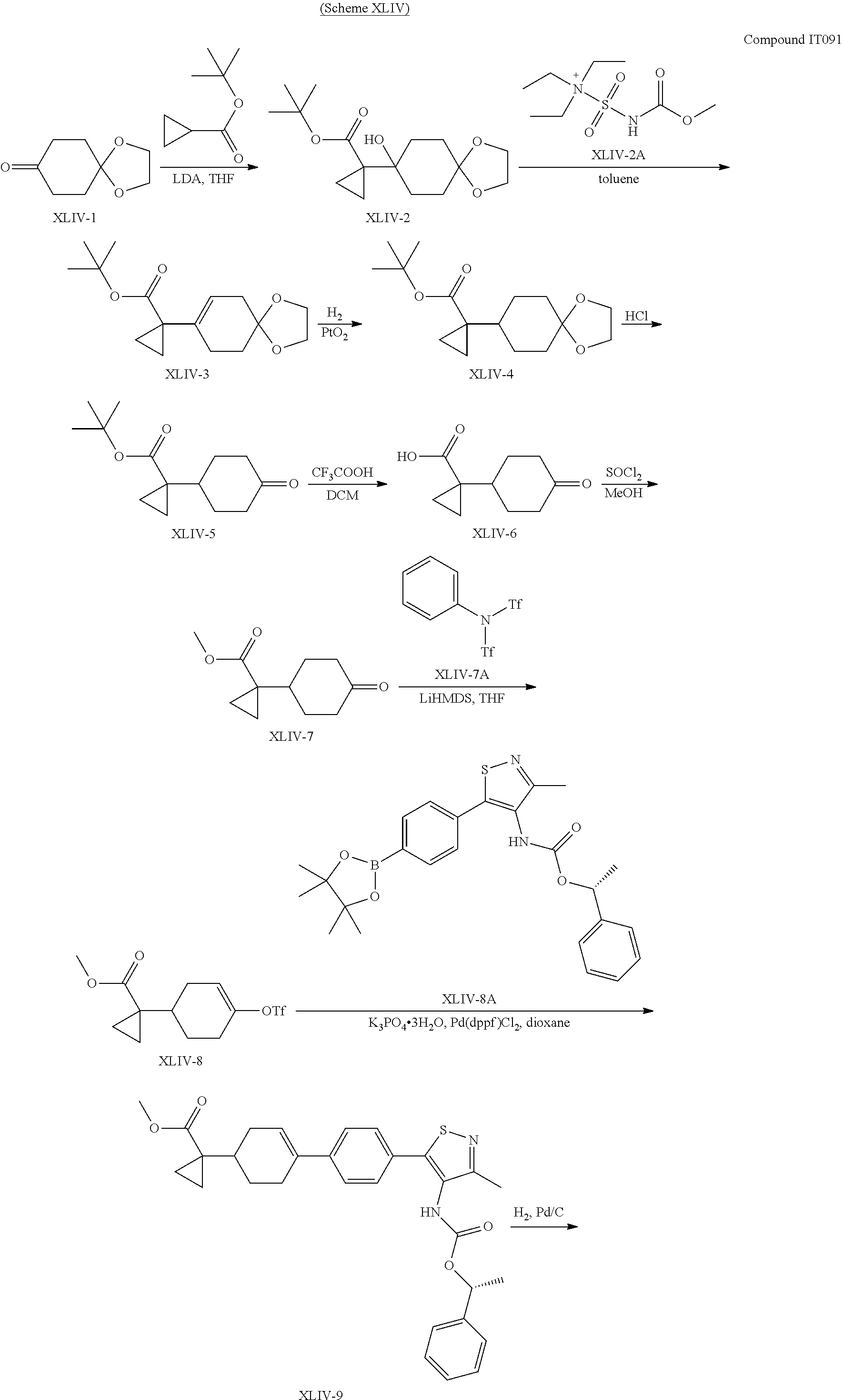

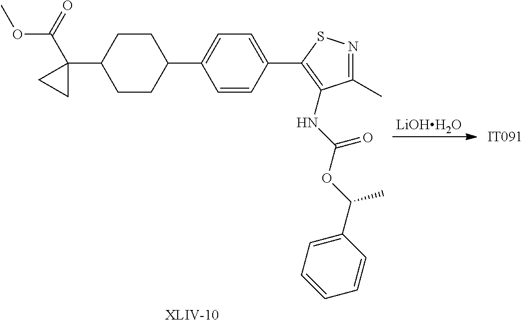

To a cooled (−78° C.) solution of 2M LDA in THF (1.4 mL, 2.8 mmol) was added tert-butyl cyclopropanecarboxylate (0.4 g, 2.8 mmol) in THF (5 mL). The mixture was stirred at −78° C. for 1 h. Then a solution of compound 1 (0.44 g, 2.8 mmol) in THF (5 mL) was added. The cooling bath was removed after stiffing for 30 mins, the solution was stirred at rt for 3 h. The reaction was quenched with saturated NH₄Cl and the mixrure was extracted with EA. The combined organic layers were washed and concentrated under vacuo. The residue was purified by column over silica gel (PE:EA=5/1) to give XLIV-2 (400 mg, yield 50%).

To a stirred solution of XLIV-2 (600 mg, 2 mmol) in toluene (10 mL) was added XLIV-2A (564 mg, 2.4 mmol) under N₂. The mixture was heated to reflux for 2 h. After being cooled to rt, the mixture was diluted with water and extracted with EA. The combined organic layers were washed and concentrated under vacuo. The residue was purified by column on silica gel (PE:EA=10/1) to give XLIV-3 (400 mg, yield 71%).

To a stirred solution of XLIV-3 (150 mg, 0.54 mmol) in EA (10 mL) was added PtO₂ (50 mg, 33%). Then the suspension was degassed under vacuum and purged with H₂ (50 psi) at 30° C. for 1 h. Then the solution was filtered and evaporated in vacuo to give XLIV-4 (100 mg, yield 67%).

To a solution of XLIV-4 (500 mg, 1.8 mmol) in MeOH (10 mL) was added HCl (5 mL, 6 N). Then it was stirred at rt for 2 h. The mixture was diluted with water and extracted with DCM. The organic layers were washed with brine, and concentrated under vacuo to give XLIV-5 (400 mg, yield 95.6%).

To a solution of XLIV-5 (350 mg, 1.48 mmol) in DCM (4 mL) was added CF₃COOH (4 mL). Then it was stirred at rt for 2 h. The mixture was diluted with water and extracted with DCM. The organic layers were washed with brine, and concentrated under vacuo to give XLIV-6 (250 mg, yield 88%).

To a solution of XLIV-6 (300 mg, 1.56 mmol) in MeOH (10 mL) was added SOCl₂ (187 mg, 1.56 mmol). Then it was stirred at 30° C. overnight. The mixture was diluted with water and extracted with DCM. The organic layers were washed with brine, and concentrated under vacuo to give XLIV-7 (150 mg, yield 47%).

To a solution of XLIV-7 (150 mg, 0.72 mmol) in THF (5 mL) was added LiHMDS (0.81 mL, 0.81 mmol) at −78° C. The solution was stirred at −78° C. for 1 h. Then XLIV-7A (293 mg, 0.81 mmol) in THF (5 mL) was added. The cooling bath was removed after stiffing for 30 mins, the solution was stirred at rt overnight. The reaction was quenched with saturated aq. NH₄Cl and the mixture was extracted with EA. The combined organic layers were washed with brine, and concentrated under vacuo. The residue was purified by column on silica gel (PE:EA=20/1) to give XLIV-8 (200 mg, yield 84%).

To a stirred solution of XLIV-8 (160 mg, 0.47 mmol), XLIV-8A (241 mg, 0.52 mmol), K₃PO₄.3H₂O (250 mg, 0.94 mmol) in dioxane (10 mL) was added Pd(dppf)Cl₂ (34.4 mg, 0.047 mmol) under nitrogen atmosphere. The mixture was purged with N₂ for 5 mins and heated to reflux for 4 h. After cooled, the mixture was diluted with water and extracted with DCM. The combined organic layers were washed with brine, and concentrated under vacuo. The residue was purified by column on silica gel (PE:EA=5/1) to give XLIV-9 (140 mg, yield 57%).

To a stirred solution of XLIV-9 (130 mg, 0.25 mmol) in EA (10 mL) was added Pd/C (65 mg, 50%). Then the suspension was degassed under vacuum and purged with H₂ (50 psi) at rt for 2 h. Then the solution was filtered and evaporated in vacuo to give XLIV-10 (110 mg, yield: 67%).

IT091 was obtained by LiOH hydrolysis of XLIV-10 (7.5 mg, yield: 7.7%). MS (ESI) m/z (M+H)⁺ 505.2. ¹H NMR (CDCl₃, 400 MHz): δ 7.20-7.39 (m, 7H), 7.12-7.14 (d, J=8.0 Hz, 2H), 6.06 (br, 1H), 5.78 (br, 1H), 2.99 (br, 1H), 2.30 (s, 3H), 2.12-2.15 (m, 2H), 1.71-1.84 (m, 3H), 1.32-1.49 (m, 7H), 1.09-1.19 (m, 2H), 0.68-0.76 (m, 2H).

IT092 was prepared by Suzuki Coupling of XLIV-8 with XIII-9 using the similar procedure described in the alternative synthesis of XIII-6, followed by standard LiOH hydrolysis. ¹H NMR (Methanol-d₄, 400 MHz): δ 7.47 (s, 1H), 7.32-7.38 (m, 5H), 6.00 (s, 1H), 5.83-5.85 (m, 1H), 3.66 (s, 3H), 2.12-2.23 (m, 4H), 1.70-1.77 (m, 1H), 1.51-1.59 (m, 3H), 1.31-1.33 (m, 2H), 1.16 (m, 2H), 0.80 (m, 2H). MS (ESI) m/z (M+H)⁺ 434.2.

### Example 37

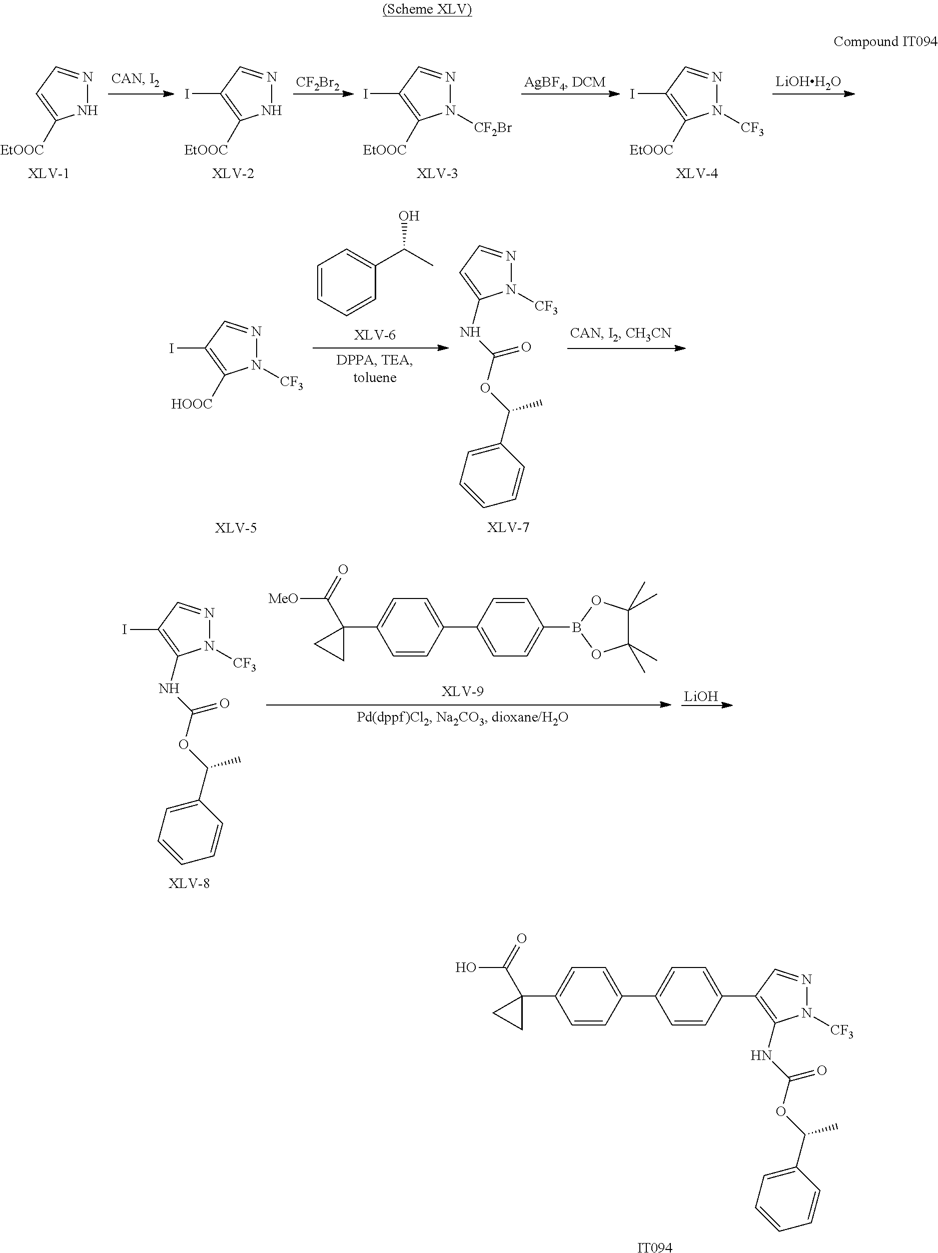

The solution of XLV-1 (10 g, 71.4 mmol), CAN (39.1 g, 71.4 mmol), I₂ (18 g, 71.4 mmol) in CH₃CN (182 mL) was stirred at 25° C. for 15 h. Then the solution of NaHSO₃ was added until the mixture was light yellow, then extracted with EtOAc, dried over Na₂SO₄, concentrated and purified by column (PE/EA=10/1) to provide XLV-2 (16 g, yield: 84%) as a white solid.

To a stirred solution of XLV-2 (20 g, 75.2 mmol) in DMF (250 mL) was added NaH (4.5 g, 112.8 mmol) at 0° C. After 1 h, CF₂Br₂ (31.3 g, 150.4 mmol) was added, then the mixture was stirred at 25° C. overnight. The mixture was quenched with water and extracted with EtOAc. The organic layer was dried over Na₂SO₄, concentrated under vacuo and purified by column (PE/EA=50/1) to provide XLV-3 (2 g, yield: 6.8%).

To a stirred solution of XLV-3 (2 g, 5.07 mmol) in DCM (30 mL) was added AgBF₄ (1.8 g, 10.14 mmol) at −78° C. Then the solution was stirred at rt for 10 h. The mixture was diluted with DCM, filtered, concentrated and purified by flash column (PE/EA=3/1) to provide XLV-4 (1.9 g, yield: 97%).

To a stirred solution of XLV-4 (1.9 g, 5.69 mmol) in MeOH/H₂O (24 mL/4 mL) was added LiOH.H₂O (1.4 g, 34.13 mmol). The mixture was stirred at rt for 30 mins. Then MeOH was removed, HCl (6N) was added to adjust pH<3, and extracted with EtOAc. The organic layer was separated, dried and concentrated to provide XLV-5 (1.5 g, yield: 88%).

A mixture of XLV-5 (100 mg, 0.33 mmol), XLV-6 (48 mg, 0.39 mmol), DPPA (107 mg, 0.39 mmol) and TEA (67 mg, 0.66 mmol) in toluene (5 mL) was stirred at 90° C. for 3 h. The toluene was removed and diluted with EtOAc and washed with water. The organic layer was dried over Na₂SO₄, concentrated and purified by column (PE/EA=3/1) to provide XLV-7 (80 mg, yield: 81%).

To a stirred solution of XLV-7 (25 mg, 0.14 mmol) in CH₃CN (5 mL) was added CAN (74 mg, 0.14 mmol) and I₂ (35 mg, 0.14 mmol). The mixture was stirred at rt for 5 h. NaHSO₃ (aq.) was added to quench the solution until the solution turned light yellow, extracted with EtOAc. The organic layer was dried over Na₂SO₄, concentrated and purified by column (PE/EA=5/1) to provide XLV-8 (25 mg, yield: 40%).

IT094 was obtained by Suzuki-Coupling of XLV-8 with XLV-9, followed by LiOH hydrolysis. IT094: MS (ESI) m/z (M+H)⁺ 536.2. Sodium salt IT094a: MS (ESI) m/z (M+H)⁺ 536.2. ¹H NMR (DMSO-d₆, 400 MHz): δ 8.29 (s, 1H), 7.59 (br, 4H), 7.50-7.52 (m, 2H), 7.34-7.36 (m, 7H), 5.74 (br, 1H), 1.46 (br, 3H), 1.24 (br, 2H), 0.78 (br, 2H).

### Example 38

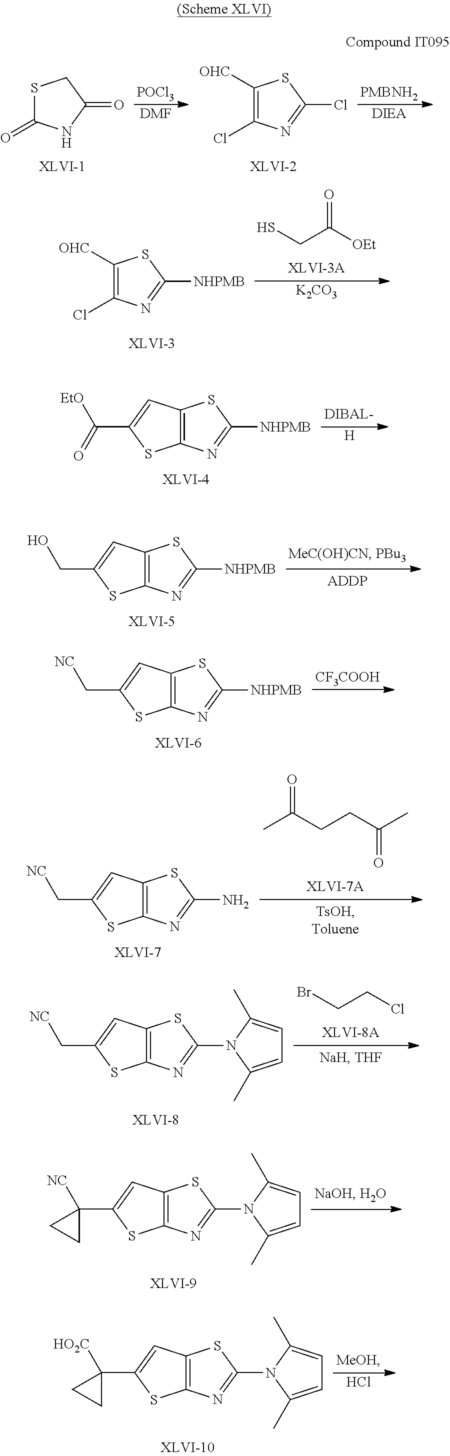

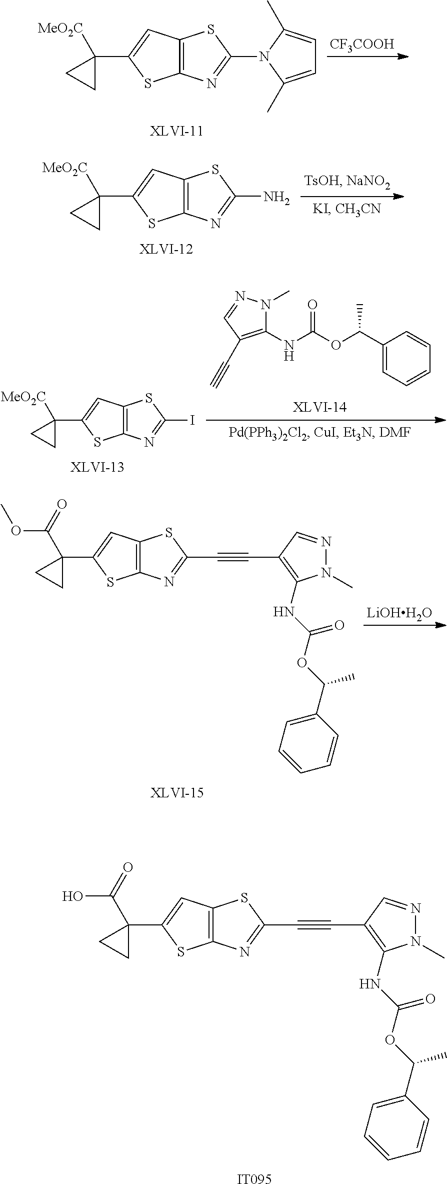

DMF (68.4 g, 940.2 mmol) was added dropwise to a suspension of XLVI-1 (100 g, 854.7 mmol) in POCl₃ (476 mL, 780 g, 1.71 mol) at 0° C. Then the mixture was stirred for 1 h at rt, then for 1 h at 85° C., after which the mixture was refluxed for 2 h. POCl₃ was removed in vacuum and the mixture was poured onto water, then extracted with DCM. The organic layer was washed with brine, dried over Na₂SO₄, concentrated in vacuum. The residue was purified by column on silica gel (PE/EA=50/1) to afford XLVI-2 (47 g, yield: 30%).

The solution of PMBNH₂ (88.5 g, 650 mmol), DIEA (226.5 mL) in THF (800 mL) was slowly added XLVI-2 (117.5 g, 650 mmol) in THF (400 mL) at rt. The mixture was stirred overnight at rt under nitrogen. THF was removed in vacuum and the mixture was washed with water and EtOAc, then filtered through a Celite pad to afford XLVI-3 (130 g, yield: 71%) without further purification.

The solution of XLVI-3 (100 g, 354.6 mmol) and K₂CO₃ (146.8 g, 1.06 mol) in DMF (1000 mL) was added XLVI-3A (51.1 g, 425.5 mmol) at rt. The mixture was heated to 120° C. and stirred for 4 hs at that temperature under nitrogen. DMF was removed in vacuum and the mixture was washed with water and EtOAc. The combined organic layers were dried over Na₂SO₄, and concentrated under reduced pressure and purified by column chromatography on silica gel (PE/EA=30/1-5/1) to afford XLVI-4 (77 g, yield: 62%).

The solution of XLVI-4 (30 g, 86.2 mmol) in 1500 mL of DCM was added DIBAL-H (431 mL, 431 mmol) at −78° C., and stirred at that temperature for 1 h under nitrogen. The mixture was quenched with NaHCO₃ (aq, 500 mL) and was diluted with EtOAc. The solution was stirred at rt for 20 mins and the resulting mixture was filtered through a Celite pad to afford XLVI-5 (23.5 g, crude yield: 90%), which used for next step without further purification.

To a solution of XLVI-5 (90 g, 294.1 mmol) and ADDP (81.5 g, 323.5 mmol) in THF (2700 mL) were added MeC(OH)CN (38.2 mL) and PBu₃ (132.5 mL) at 0° C. The mixture was stirred for 1 h at rt. The mixture was quenched with water and EtOAc. The combined organic layers were dried over Na₂SO₄, and concentrated under vacuo. The residue was purified by column chromatography on silica gel (PE/DCM=3/1-1/1) to afford XLVI-6 (30 g, yield: 32.4%).

To a solution of XLVI-6 (20 g, 63.5 mmol) in TFA (200 mL) was heated to 55° C. and stirred at that temperature for 5 h. The mixture was adjusted to pH=7-8 with NaHCO₃ (aq.) and extracted with DCM. The organic layer were washed with brine, dried over Na₂SO₄, concentrated in vacuum to afford XLVI-7 (10 g, yield: 81%), which used for next step without further purification.

To a stirred solution of XLVI-7 (11 g, 57 mmol) in toluene (400 mL) was added XLVI-7B (6.5 g, 7.81 mL, 57 mmol) and p-TsOH (541.5 mg, 2.85 mmol). After the addition, the solution was heated to 130° C. for 5 hours. Toluene was removed in vacuum and the residue was purified by column chromatography on silica gel (PE/DCM=3/1) to afford XLVI-8 (9.8 g, yield: 63%).

The solution of XLVI-8 (4.9 g, 18 mmol) in THF (60 mL) was slowly added NaH (1.4 g, 35.9 mmol) at 0° C. The mixture was stirred at rt for 1.5 h under nitrogen. Then XLVI-8A (3.4 g, 23.3 mmol) was added at 0° C. The mixture was stirred at rt for 2 h under nitrogen. The mixture was quenched with NH₄Cl (aq.), and extracted with EtOAc. The combined organic layers were dried over Na₂SO₄, and concentrated under vacuo. The residue was purified by column chromatography on silica gel (PE/EA=9/1) to afford XLVI-9 (4.5 g, yield: 83%).

To a stirred solution of XLVI-9 (2.2 g, 7.4 mmol) in MeOH (60 mL) was added NaOH (120 mL, 35% w). After the addition, the solution was heated to 85° C. overnight. MeOH was removed in vacuum and the mixture was adjusted to pH=4-5 with 4M HCl, and was extracted DCM. The organic layer was washed with brine, dried over Na₂SO₄, concentrated in vacuum to afford XLVI-10 (2.8 g, crude yield: 116%), which used for next step without further purification.

To a stirred solution of XLVI-10 (2.8 g, 8.8 mmol) in MeOH (50 mL) was added HCl (300 mg, 12M). After the addition, the solution was heated to 80° C. overnight. MeOH and HCl was removed in vacuum and the residue was purified by column chromatography on silica gel (PE/EA=9/1) to afford c XLVI-11 (1.7 g, yield: 58%).

To a solution of XLVI-11 (3.4 g, 10.2 mmol) in H₂O (40 mL) was slowly added TFA (40 mL) at 0° C. The mixture was heated to 60° C. and stirred at that temperature for 3 h. The mixture was adjusted to pH=8 with NaHCO₃ (aq.) and was extracted DCM. The organic layers were washed with brine, dried over Na₂SO₄, concentrated in vacuum to afford XLVI-12 (4.0 g, crude yield: 153%).

To a stirred solution of XLVI-12 (4.0 g, 15.7 mmol) in MeCN (50 mL) was added TsOH (9.0 g, 47.2 mmol). Then NaNO₂ (2.1 g, 31.4 mmol), KI (6.5 g, 39.3 mmol) dissolved in H₂O (30 mL) was added dropwise at 0° C. After the addition, the solution was stirred at rt for 4 h. MeCN was removed in vacuum and the reaction mixture was extracted EtOAc. The organic layers were washed with brine, dried over Na₂SO₄, concentrated in vacuum. The residue was purified by column chromatography on silica gel (PE/EA=10/1) to afford XLVI-13 (1.7 g, yield: 30%).

XLVI-15 and IT095 were prepared following the similar procedure described in the preparation of 1-6 and IT001. IT095: MS (ESI) m/z (M+H)⁺ 493.1. ¹H NMR (Methanol-d₄, 400 MHz): δ 7.66 (s, 1H), 7.35-7.37 (m, 2H), 7.26-7.29 (m, 2H), 7.19-7.21 (m, 2H), 5.80-5.85 (q, J=6.4 Hz, 1H), 3.67 (s, 1H), 1.73-1.76 (m, 2H), 1.56-1.58 (d, J=6.4 Hz, 3H), 1.44-1.46 (m, 2H).

IT102 was prepared by following the similar procedure described in the synthesis of IT095 using (R)-1-phenylethyl (5-ethynyl-3-methylisoxazol-4-yl)carbamate in place XLVI-14 in the Suzuki-Coupling with XLVI-13. ¹H NMR (Methanol-d₄, 400 MHz): δ 7.25-7.42 (m, 6H), 5.86 (d, J=6.4 Hz, 1H), 2.24 (s, 3H), 1.81 (brs, 2H), 1.59-1.61 (d, J=6.4 Hz, 3H), 1.51 (brs, 2H). MS (ESI) m/z (M+H)⁺ 494.2.

### Example 39

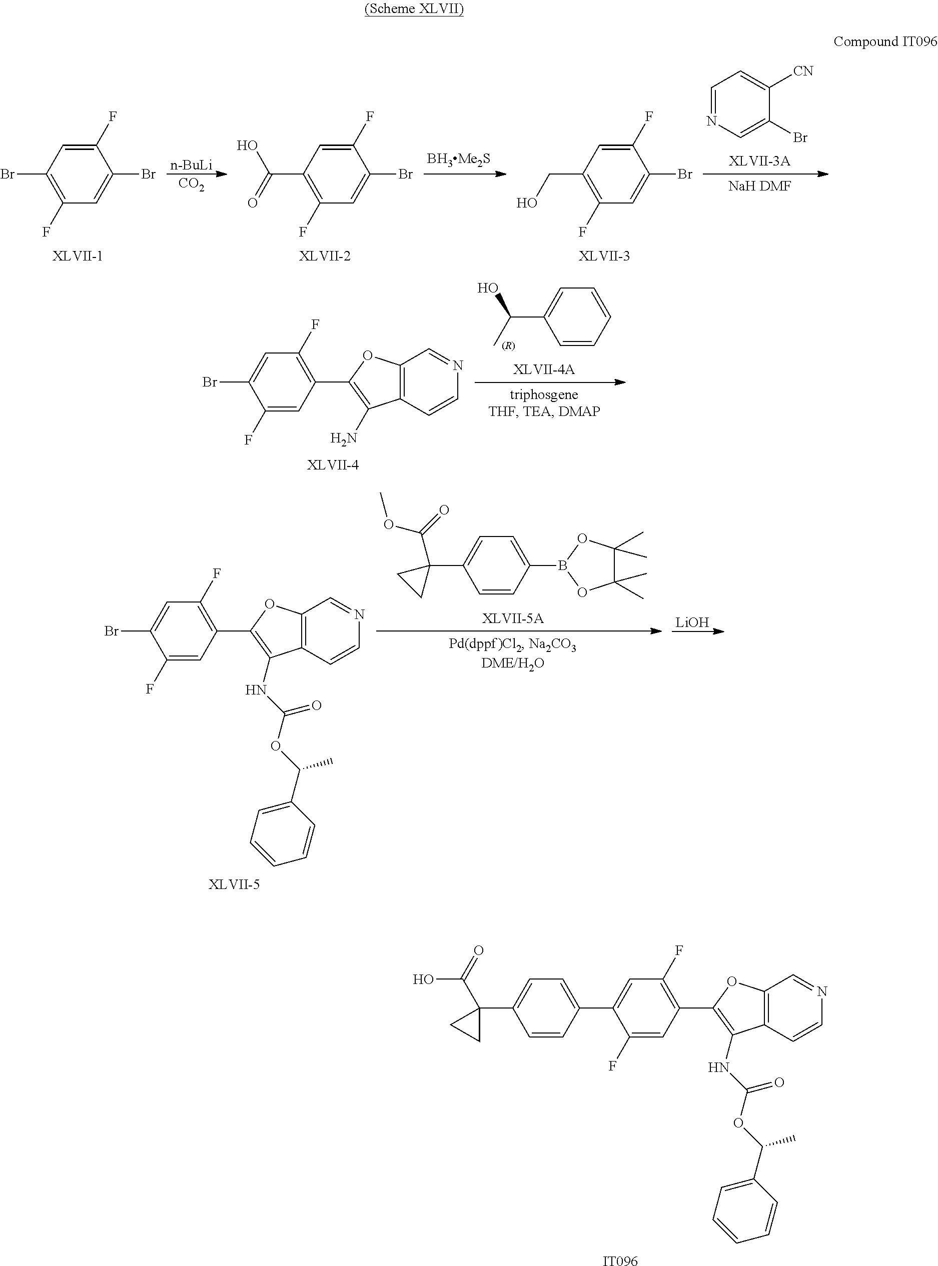

To the solution of XLVII-1 (13.55 g, 50 mmol), in Et₂O (150 mL) was added n-BuLi (2.5 N, 20 mL) at −78° C. The reaction mixture was stirred at −78° C. under Ar for 30 min and CO₂ was bubbled into the solution. The mixture was warmed up to rt. The precipitate was collected by filtration and washed with Et₂O. The obtained solid was treated with water and HCl (1N) to pH=2. The mixture was extracted with t-BuOMe. The combined organic layers were washed with brine, dried over MgSO₄, and concentrated to afford XLVII-2 (10.0 g, yield 84.4%), which was used next step without purification.

The mixture of XLVII-2 (2.37 g, 10 mmol) in THF (25 mL) was added BH₃.Me₂S (10 N, 2.5 mL) at rt under N₂, The mixture was heated to reflux for 2 h and quenched with adding MeOH and diluted with EtOAc. The organic layer was washed with brine, dried over MgSO₄ and concentrated under vacuo then purified by chromatography on silica gel (PE/EA=3/1) to afford XLVII-3 (1.50 g, yield 67.3%).

NaH (210 mg, 8.8 mmol) was added to a solution of XLVII-3 (446 m g, 2 mmol) in DMF (10 mL) at 0° C. The reaction mixture was stirred at 0° C. for 30 mins. A solution of XLVII-3A (366 mg, 2 mmol) in DMF (5 mL) was added dropwise. The reaction mixture was stirred at 0° C. for 4 h. Water (5 mL) was added. The reaction mixture was diluted with brine and EtOAc. The aqueous layer was extracted with EtOAc. The combined organic layer was washed with brine, dried over MgSO₄ and concentrated. The crude product was purified by column (PE/EA=2/1) to afford XLVII-4 (200 mg, yield 30.7%).

XLVII-5 and IT096 were prepared following the similar procedure described in the preparation of XXI-3 and IT031. IT096: ¹H NMR (Methanol-d₄, 400 MHz): δ 9.16 (s, 1H), 8.51 (s, 1H), 7.95 (s, 1H), 7.70-7.73 (m, 1H), 7.60 (d, J=7.2 Hz, 2H), 7.53 (d, J=8.4 Hz, 2H), 7.40-7.44 (m, 4H), 7.31 (br, 2H), 5.83-5.84 (m, 2H), 1.65-1.67 (m, 5H), 1.27-1.30 (m, 2H). MS (ESI) m/z (M+H)⁺ 555.1.

### Example 40

### General Synthetic Scheme for Exemplary Compounds of Formula (II)

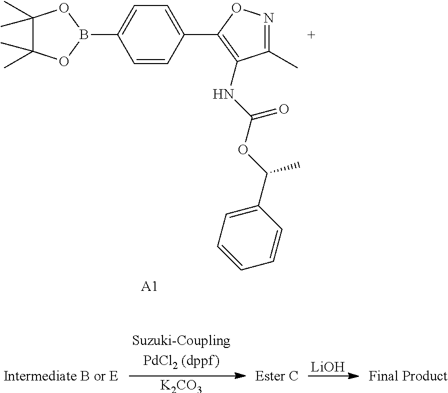 [SMILES: Cc1noc(-c2ccc(B3OC(C)(C)C(C)(C)O3)cc2)c1NC(=O)O[C@H](C)c1ccccc1 ; "A1" ; "LiOH" ; "K2CO3"]

### Synthesis of Intermediate B1

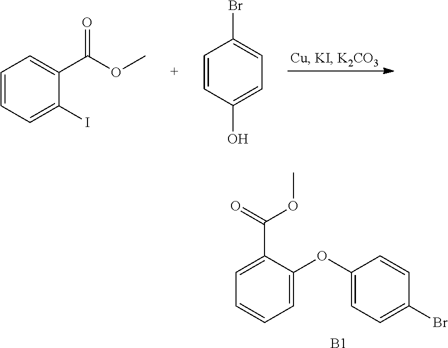

Methyl 2-iodobenzoate (550 mg, 2.1 mmol), 4-bromophenol (700 mg, 4.0 mmol), potassium carbonate (310 mg, 2.2 mmol), Cu powder (128 mg, 2.1 mmol) and KI (160 mg, 1.0 mmol) were combined in 18 mL dry DMF. The resulting mixture was heated at 115° C. in a sealed tube for 24 hrs. The reaction mixture was diluted with 80 mL EA, washed with 3×40 mL water and brine. The crude mixture was purified on ISCO silica gel column to afford an oil B1 (400 mg, 1.3 mmol).

### Synthesis of Intermediate Compound B2

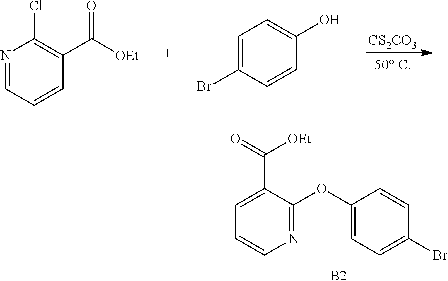

Ethyl 2-chloronicotinate (500 mg, 2.69 mmol), 4-bromophenol (466 mg, 2.69 mmol) and cesium carbonate (1.75 g, 5.4 mmol) were combined in 8 mL dry DMF. The mixture was heated at 55° C. in a sealed tube overnight. The mixture was diluted with 50 mL EA, washed with 3×30 mL water and brine. The crude mixture was purified on ISCO silica gel column to afford B2 (725 mg, 2.25 mmol) as a white solid.

### Synthesis of Intermediate Compound B3

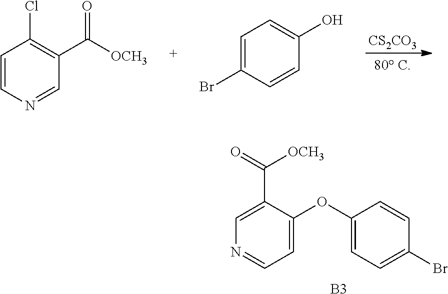

Methyl 4-chloronicotinate (1.0 g, 5.38 mmol), 4-bromophenol (0.94 g, 5.38 mmol) and cesium carbonate (3.5 g, 10.78 mmol) were combined in 15 mL dry DMF. The mixture was heated at 80° C. in a sealed tube overnight. The mixture was diluted with 80 mL EA, washed with 3×50 mL water and brine. The crude mixture was purified on ISCO silica gel column to afford B3 (1.37 g, 4.25 mmol) as a yellow solid.

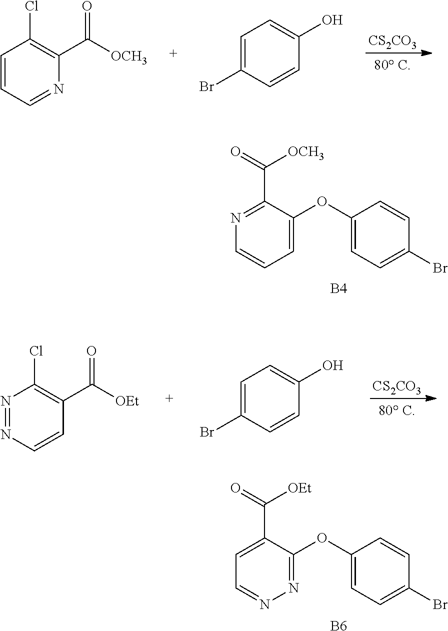

B4 and B6 were prepared by following the same procedure for the synthesis of B3.

### Synthesis of Intermediate Compound B5

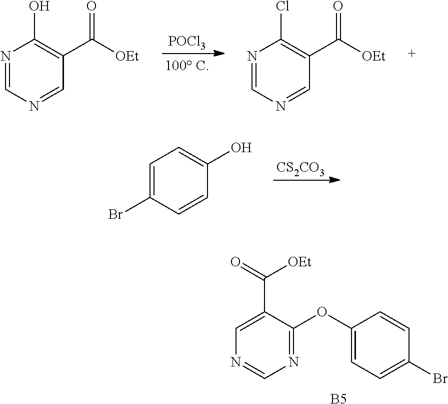

Ethyl 4-hydroxypyrimidine-5-carboxylate (1.0 g, 5.95 mmol) was dissolved in 15 mL POCl₃. The mixture was heated at 100° C. for 2 hrs in a sealed tube. POCl₃ was removed in vacuo, the residue was dissolved in 60 mL EA, washed with 50 mL ice-water and 30 mL saturate NaHCO₃ and brine. The organic phase was dried over Na₂SO₄, and then concentrated in vacuo to afford brown oil ethyl 4-chloropyrimidine-5-carboxylate (1.1 g, 5.91 mmol) that was used without further purification.

4-bromophenol (0.46 g, 2.68 mmol), cesium carbonate (1.74 g, 5.36 mmol) were combined in 8 mL dry DMF, the mixture was cooled with ice-water bath for 5 mins. Ethyl 4-chloropyrimidine-5-carboxylate (0.5 g, 2.68 mmol) in 2 mL DMF was added dropwise. The resulting mixture was stirred at 5° C. for 1 hr; the reaction mixture was diluted with 80 mL EA, washed with 3×40 mL water and brine. The organic phase was dried over Na₂SO₄ and concentrated in vacuo to afford a brown solid. The crude product was purified on ISCO Silica Gel column to afford a yellow solid B5 (0.625 g, 1.93mmoL).

### Synthesis of Intermediate Compound B7

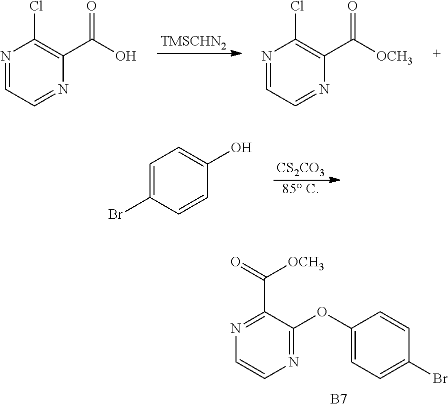

3-chloropyrazine-2-carboxylic acid (1.0 g, 6.3mmoL) was dissolved in 10 mL CH₂Cl₂ and 4 mL MeOH. A solution of 2M TMSCHN₂ in hexane (5 mL, 10mmoL) was added dropwise. The mixture was stirred at rt for 15mins. The solvent was removed in vacuo to afford oil methyl 3-chloropyrazine-2-carboxylate (1.05 g, 6.08 mmol) which was used without further purification.

Methyl 3-chloropyrazine-2-carboxylate (0.5 g, 2.89 mmol), 4-bromophenol (0.5 g, 2.89 mmol) and cesium carbonate (1.88 g, 5.78 mmol) were combined in 10 mL dry DMF. The mixture was heated at 85° C. for 2 hrs. The mixture was diluted with 80 mL EA, washed with 3×50 mL water and brine. The crude mixture was purified on ISCO silica gel column to afford B7 (0.68, 2.21 mmol) as a white solid.

### Synthesis of Intermediate Compound B8

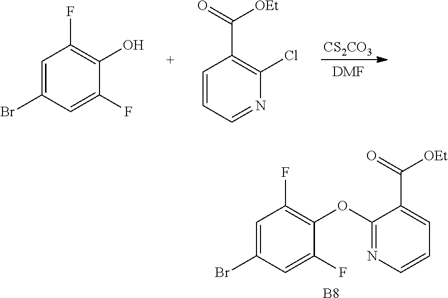

4-bromo-2,6-difluorophenol (0.56 g, 2.68 mmol) was dissolved in 8 mL dry DMF, then added CS₂CO₃ (1.8 g, 5.35 mmol) and ethyl 2-chloronicotinate (0.5 g, 2.68 mmol). The resulting mixture was heated at 135° C. for 18 h in a sealed tube. The mixture was diluted with EA, washed with water and brine. The crude mixture was purified on ISCO to afford B8 as a white solid (0.38 g, 1.06 mmol).

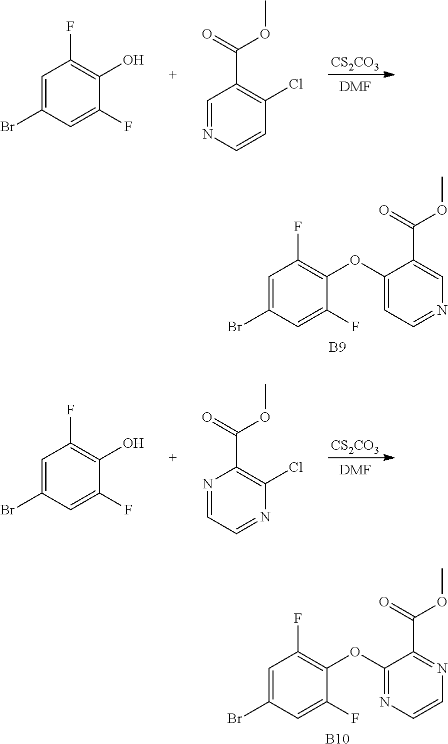

B9 and B10 were prepared by following the same procedure for the synthesis of B8.

### Synthesis of Intermediate B11

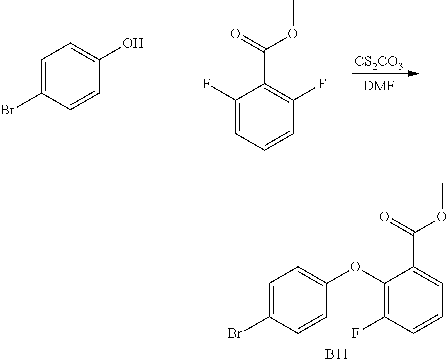

4-bromophenol (1.0 g, 5.78 mmol) and Cs₂CO₃ (3.75 g, 11.56 mmol) were combined in 15 mL dry DMF. The mixture was stirred at rt for 5 mins under N₂. Then methyl 2,6-difluorobenzoate (1.0 g, 5.78 mmol) was added. The resulting mixture was heated at 130° C. for 2 hrs. The mixture was diluted with 50 mL EA and 20 mL hexane, and then washed with water and brine. The crude mixture was purified to afford B11 (1.1 g, 3.38 mmol).

Intermediate B12 was prepared following the similar procedure described in the synthesis of B11 using methyl 2,5-difluorobenzoate instead.

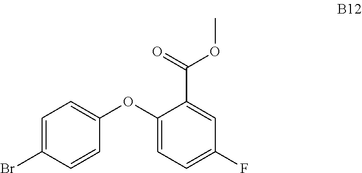 [SMILES: COC(=O)c1cc(F)ccc1Oc1ccc(Br)cc1]

### Synthesis of Intermediate B13

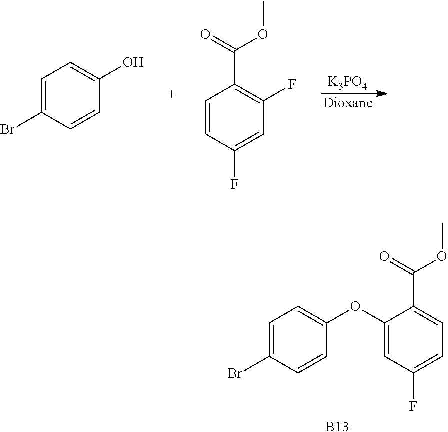

4-bromophenol (1.0 g, 5.78 mmol) and K₃PO₄ (1.5 g, 7.0 mmol) were combined in 15 mL dry dioxane. The mixture was stirred at rt for 5 mins under N₂. Then methyl 2,4-difluorobenzoate (1.0 g, 5.78 mmol) was added. The resulting mixture was heated at 115° C. for overnight. The solvent was removed in vacuo, the residue was diluted with 50 mL EA and 20 mL hexane, and then washed with water and brine. The crude mixture was purified to afford B13 as a white solid (0.64 g, 1.97 mmol).

### Synthesis of Intermediate B14

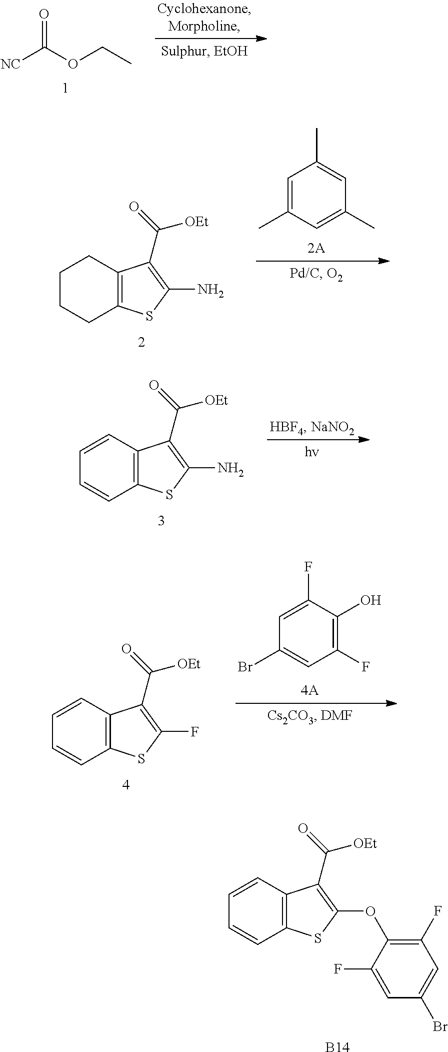

The mixture of compound 1 (30 g, 0.265mol) and cyclohexanone (31.2 g, 0.32mol), Morpholine (28 g, 0.32 mol), sulfur (10.2 g, 0.32 mol) in EtOH (300 mL) was stirred for 18 hrs at 50° C. The reaction was cooled to rt. The appeared solid was collected by filtration. The solid was washed with cold EtOH to give compound 2 (40 g, yield 67.1%) as a yellow solid.

To a stirred solution of compound 2 (5 g, 22.2 mmol) in compound 2A (100 mL) was added Pd/C (5 g). The mixture was stirred at 110° C. for 48 hrs under O₂. Filtered and the filtrate partitioned between EA and water. The organic layer was subject to standard work-up procedure and purified to afford compound 3 (1.2 g, yield 24.5%) as a yellow solid.

To a stirred solution of compound 3 (866 mg, 3.92 mmol) in 48% HBF₄ (24 mL) was cooled to −12° C. and a solution of NaNO₂ (351.6 mg, 5.1 mmol) in H₂O (1 mL) was added dropwise with stiffing. After 15 mins, the reaction mixture was transferred to a photochemical reaction flask and filled with HBF₄ (200 mL). The solution was cooled to −7° C. and irradiated for 4 hrs. The solution was neutralized 50% NaOH (200 mL) at −78° C. and warming to rt and extracted with EA. The combined organic layer was dried over Na₂SO₄, concentrated and purified to afford compound 4 (20 mg, yield 2.27%) as a yellow solid.

A mixture of compound 4 (10 mg, 0.045 mmol) and Cs₂CO₃ (9.3 mg, 0.045 mmol) in DMF (2 mL) was stirred at 120° C. for 1 h. The mixture was poured into water and extracted with EA. The combined organic layers were subject to standard work-up procedure and purified to give B14 (5 mg, yield: 28%).

### Synthesis of Intermediate B15

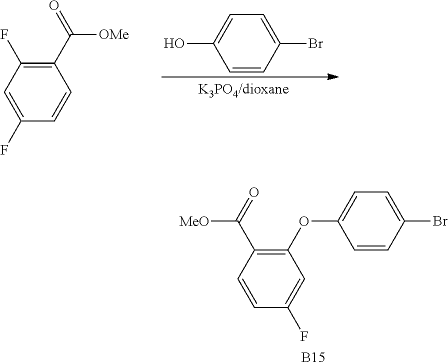

4-bromophenol (80 g, 0.465 mmol) and K₃PO₄ (120 g, 0.56 mmol) were combined in 1200 mL dry dioxane. The mixture was stirred at rt for 5 min. under N₂ protection. Then methyl 2,4-difluorobenzoate (80 g, 0.465 mmol) was added. The mixture was heated to reflux under nitrogen overnight. After concentrated, the residue was partitioned between H₂O and EA, the aqueous phase was extracted with EA, and the combined organic layer was washed with brine, dried over Na₂SO₄, and concentrated. The residue was purified to afford B15 (65 g, yield: 43.33%).

### Synthesis of Intermediate E1

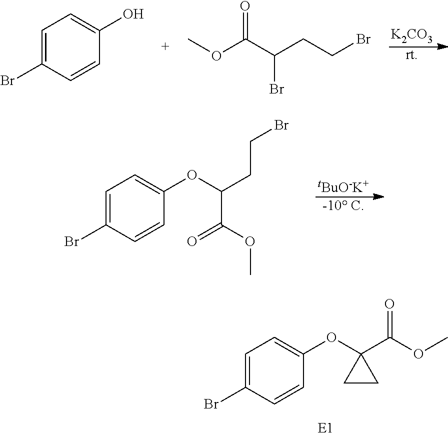

4-Bromophenol (1.0 g, 5.78 mmol) was dissolved in 10 mL dry DMF, added potassium carbonate (1.2 g, 11.56 mmol). The mixture was stirred at rt for 10 mins. To the mixture was added methyl 2,4-dibromobutanoate (1.5 g, 5.78 mmol) dropwise. The resulting mixture was stirred at rt for 3 hrs. The mixture was diluted with 60 mL EA, removed inorganic solid by filtration, then washed with 3×40 mL water, and brine. The organic phase was dried over Na₂SO₄. The crude mixture was purified on ISCO Silica Gel column to afford an intermediate oil methyl 4-bromo-2-(4-bromophenoxy)butanoate (1.41 g, 4.0 mmol).

Methyl 4-bromo-2-(4-bromophenoxy)butanoate (0.355 g, 1mmol) was dissolved in 10 mL dry THF under N₂, cooled with ice-acetone bath. To the mixture was added solid KOtBu (0.115 g, 1.0 mmol) in portions. The resulting mixture was stirred at −10° C. for 30mins, then at room temperature for 2 hrs. The reaction was dried in vacuo, the residue was directly purified on ISCO silica gel column to afford clear oil E1 (105 mg, 0.38 mmol).

E5 was prepared similarly as E1 using 4-bromo-2,6-difluorophenol as the starting material.

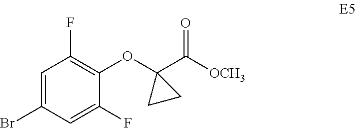 [SMILES: COC(=O)C1(Oc2c(F)cc(Br)cc2F)CC1]

### Synthesis of Intermediate E2

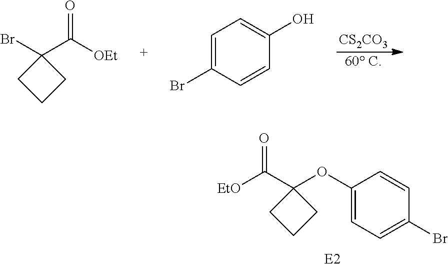

4-bromophenol (0.5 g, 2.89 mmol), cesium carbonate (1.88 g, 5.78 mmol) were combined in 8 mL dry DMF, the mixture was heated at 60° C. Ethyl 1-bromocyclobutanecarboxylate (1.2 g, 5.78 mmol) in 3 mL DMF was added dropwise. The resulting mixture was stirred at 60° C. for 5 hrs. The reaction mixture was diluted with 80 mL EA, washed with 3×40 mL water and brine. The organic phase was dried over Na₂SO₄ and concentrated in vacuo. The crude product was purified on ISCO Silica Gel column to afford oil E2 (0.15 g, 0.5mmoL).

Intermediates E3 and E4 were prepared following the similar procedure described in the synthesis of E2.

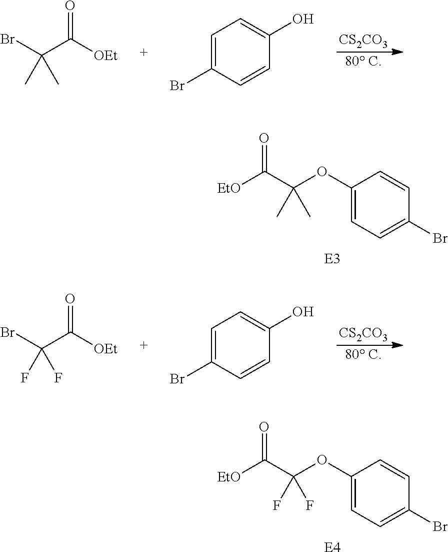

### Synthesis of Intermediate E6

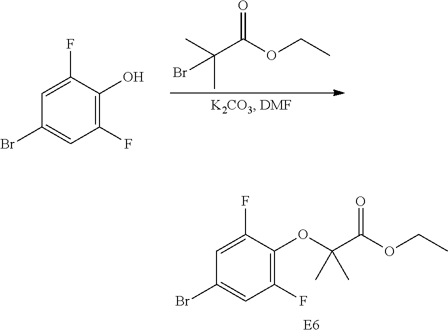

A mixture of 4-bromo-2,6-difluorophenol (500 mg, 2.4 mmol), ethyl 2-bromo-2-methylpropanoate (466 mg, 2.4 mmol) and K₂CO₃ (662 mg, 4.8 mmol) in DMF (5 mL) was stirred at 23° C. for 2 hrs. H₂O (10 mL) was added and the reaction mixture was extracted with EA. The organic layer was washed with H₂O, dried over Na₂SO₄, concentrated and purified to provide E6 (500 mg, yield: 64.7%) as a clear oil.

Intermediate E7 was prepared similarly as E6 using 4-bromo-2-fluorophenol as starting material.

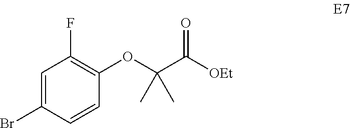 [SMILES: CCOC(=O)C(C)(C)Oc1ccc(Br)cc1F]

### Synthesis of Compound IT155

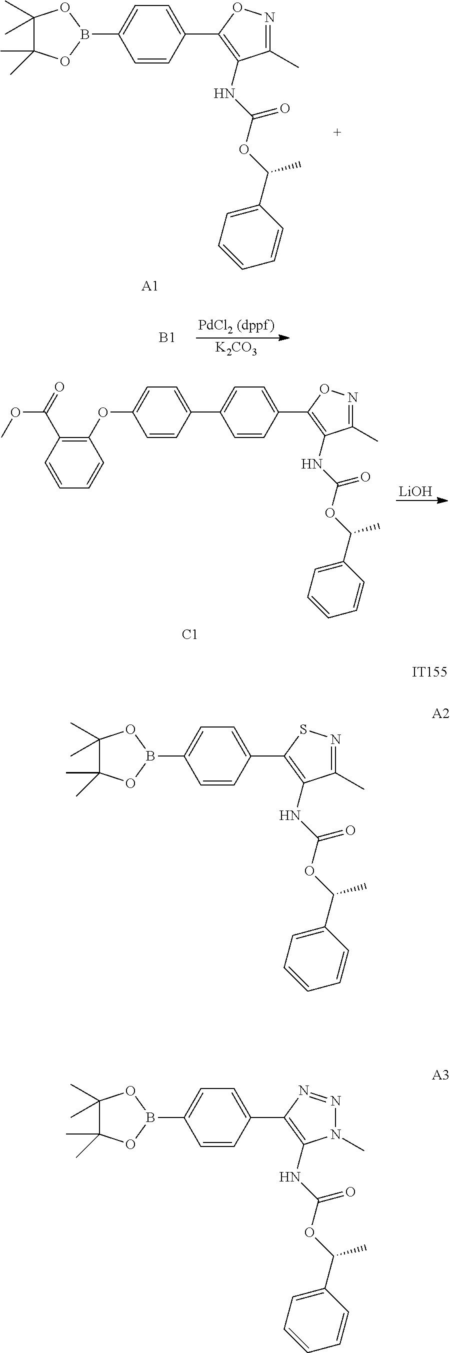

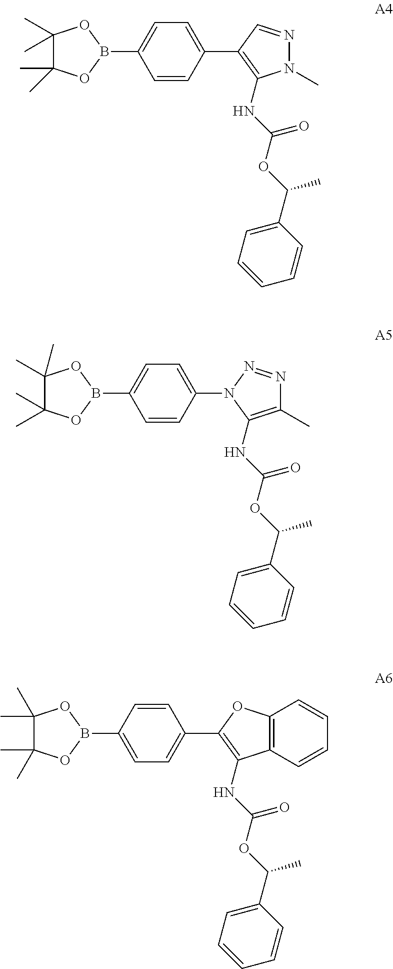

(R)-1-phenylethyl (5-(4-bromophenyl)-3-methylisoxazol-4-yl)carbamate (1.0 g, 2.49 mmol), 4,4,4′,4′,5,5,5′,5′-octamethyl-2,2′-bi(1,3,2-dioxaborolane) (0.7 g, 2.74 mmol) and potassium acetate (0.37 g, 3.74 mmol) were combined in 10 mL dry 1,4-dioxane. The mixture was flushed with N₂, then added PdCl₂(dppf)CH₂Cl₂ (183 mg, 0.25 mmol). The resulting mixture was heated at 80° C. for 3 hrs in a sealed-tube. The reaction mixture was diluted with 30 mL ethyl acetate. The precipitated solid was removed by filtration. The solvent was concentrated in vacuum to afford dark oil, which was directly purified on ISCO silica gel column to provide intermediate A1 (0.82 g, 1.83 mmol) as white solid.

Intermediates A2, A3, A4, A5 and A6 were prepared following similar procedure described in the synthesis of A1 using the corresponding carbamates.

Intermediate A1 (440 mg, 0.98 mmol) and Intermediate B1 (300 mg, 0.98 mmol) were dissolved in 10 mL 1,4-dioxane, then added 2M K₂CO₃ in water (2 mL). The mixture was flushed with N₂, then added PdCl₂(dppf)CH₂Cl₂ (172 mg, 0.22 mmol). The resulting mixture was heated at 90° C. for 2 hrs in a sealed-tube. The reaction mixture was then diluted with 50 mL ethyl acetate, washed with water and brine. The solvent was concentrated in vacuum to afford dark oil, which was directly purified on ISCO silica gel column to provide Ester C1 (285 mg, 0.052 mmol) as a light yellow solid.

Ester C1 (285 mg, 0.052 mmol) was dissolved in 10 mL MeOH and 5 mL THF. To the solution was added 2N LiOH (4 mL). The resulting mixture was stirred at rt. for 1 hr. The solvent was removed in vacuo, the residue was diluted with 20 ml water, then adjusted pH to 1 by adding 2N HCl. The white precipitate was collected by filtration, washed with 2×10 ml water, dried in high vacuum to afford IT155 (250 mg, 0.047 mml) as a white solid. Sodium salt IT155a: ¹H NMR (Methanol-d₄, 400 MHz): δ 7.65-7.76 (m, 1H), 7.56-7.63 (m, 5H), 7.30-7.42 (m, 5H), 7.06-7.13 (m, 4H), 6.94 (d, J=8.0 Hz, 1H), 5.80 (brs, 1H), 2.16 (s, 3H), 1.59 (d, J=6.4 Hz, 1H). MS (ESI) m/z (M+H)⁺ 535.2.

Compound IT197 was prepared following the same procedure described in the synthesis of IT155 by Suzuki-Coupling of intermediate E1 and A1, followed by LiOH hydrolysis to afford the final product IT197.

(R)-1-phenylethyl (3-methyl-5-(4-(4,4,5,5-tetramethyl-1,3,2-dioxaborolan-2-yl)phenyl)isothiazol-4-yl)carbamate (A2) was prepared by following the similar procedure described in the synthesis of Intermediate A1. IT177 was prepared by the Suzuki-Coupling of A2 with methyl 2-bromothiazole-5-carboxylate and subsequent LiOH hydrolysis following the similar procedure described in the synthesis of IT155. IT177: MS(ESI) m/z (M+H)⁺ 466.1. ¹H NMR (Methanol-d₄, 400 MHz): δ 8.42 (s, 1H), 8.02 (d, J=8.0 Hz, 1H), 7.58 (d, J=7.6 Hz, 2H), 7.01-7.37 (m, 6H), 5.76 (br, 1H), 2.34 (s, 3H), 1.56 (d, J=6.4 Hz, 3H).

IT202 was prepared by the Suzuki-Coupling of (R)-1-phenylethyl (3-methyl-5-(4-(4,4,5,5-tetramethyl-1,3,2-dioxaborolan-2-yl)phenyl)isothiazol-4-yl)carbamate (A2) with methyl 1-(5-bromothiazol-2-yl)cyclopropanecarboxylate and subsequent LiOH hydrolysis following the similar procedure described in the synthesis of IT155. MS (ESI) m/z (M+H)⁺ 506.1. ¹H NMR (Methanol-d₄, 400 MHz): δ 8.00 (s, 1H), 7.51-7.64 (m, 5H), 7.38-7.40 (m, 4H), 7.03-7.20 (m, 1H), 5.77-5.79 (br, 1H), 2.35 (s, 3H), 1.95-1.97 (m, 2H), 1.83-1.84 (m, 2H), 1.58-1.59 (m, 3H).

IT209 was prepared following the similar procedure for the synthesis of IT177 using (R)-1-phenylethyl (1-methyl-4-(4-(4,4,5,5-tetramethyl-1,3,2-dioxaborolan-2-yl)phenyl)-1H-1,2,3-triazol-5-yl)carbamate (A3) in place of the isothiazolyl carbamate analaog A2. MS (ESI) m/z (M+H)⁺ 450.1. ¹H NMR (Methanol-d₄, 400 MHz): δ 8.40 (s, 1H), 8.02 (d, J=8.4 Hz, 2H), 7.83 (d, J=8.0 Hz, 2H), 7.40 (s, 5H), 5.84 (br, 1H), 3.92 (s, 3H), 1.63 (s, 3H).

IT210 was prepared by the Suzuki-Coupling of methyl 1-(2-iodothiazol-5-yl)cyclopropanecarboxylate with (R)-1-phenylethyl (3-methyl-5-(4-(4,4,5,5-tetramethyl-1,3,2-dioxaborolan-2-yl)phenyl)isothiazol-4-yl)carbamate (A2) and subsequent LiOH hydrolysis following the similar procedure described in the synthesis of IT155. IT210: MS (ESI) m/z (M+H)⁺506.1. ¹H NMR (Methanol-d₄, 400 MHz): δ 9.06 (s, 1H), 7.91 (d, J=7.6 Hz, 2H), 7.70 (s, 1H), 7.55 (d, J=7.6 Hz, 2H), 7.29-7.37 (m, 5H), 5.76 (br, 1H), 2.33 (s, 3H), 1.79-1.82 (m, 2H), 1.56 (d, J=6.4 Hz, 3H), 1.46-1.49 (m, 2H).

IT237 was prepared by the Suzuki-Coupling of methyl 1-(5-bromothiophen-2-yl)cyclopropanecarboxylate with (R)-1-phenylethyl (3-methyl-5-(4-(4,4,5,5-tetramethyl-1,3,2-dioxaborolan-2-yl)phenyl)isothiazol-4-yl)carbamate (A2) and subsequent LiOH hydrolysis following the similar procedure described in the synthesis of IT155. MS (ESI) m/z (M+H)⁺ 505.1. ¹H NMR (Methanol-d₄, 400 MHz): δ 7.56-7.58 (m, 2H), 7.41-7.43 (m, 2H), 7.33-7.35 (m, 4H), 7.25-7.30 (m, 1H), 7.24 (s, 1H), 6.93-6.94 (d, J=4.0 Hz, 1H), 5.74-5.75 (br, 1H), 4.56 (s, 1H), 2.29 (s, 3H), 1.68-1.71 (m, 2H), 1.53-1.55 (d, J=6.4 Hz, 3H), 1.34-1.37 (m, 2H).

IT238 and IT256 were prepared following the same procedure described in the synthesis of IT155 by Suzuki-Coupling of intermediate ethyl 2-(4-bromophenoxy)propanoate and A1, LiOH hydrolysis, followed by prep-SFC separation. MS (ESI) m/z (M+H)⁺ 487.1. IT238: ¹H NMR (DMSO-d₆, 400 MHz): δ 9.30 (s, 1H), 7.76-7.77 (m, 4H), 7.68 (d, J=8.4 Hz, 2H), 7.34-7.43 (m, 5H), 6.99 (d, J=7.6 Hz, 2H), 5.76 (d, J=6.4 Hz, 1H), 4.88-4.91 (t, J=6.4 Hz, 1H), 2.12 (s, 3H), 1.54 (br, 3H), 1.52 (br, 3H). IT256: ¹H NMR (DMSO-d₆, 400 MHz): δ 9.30 (s, 1H), 7.75-7.77 (m, 4H), 7.67 (d, J=8.4 Hz, 2H), 7.33-7.43 (m, 5H), 6.99 (d, J=8.4 Hz, 2H), 5.76 (d, J=6.8 Hz, 1H), 4.89 (d, J=6.4 Hz, 1H), 2.12 (s, 3H), 1.54 (br, 3H), 1.52 (br, 3H).

IT258 was prepared following the same procedure described in the synthesis of IT155 by Suzuki-Coupling of B8 and A1, followed by LiOH hydrolysis to afford the final product IT258.

IT277 was prepared following the same procedure described in the synthesis of IT155 by Suzuki-Coupling of intermediate ethyl 2-(4-bromophenoxy)acetate and A1, followed by LiOH hydrolysis to afford the final product IT277. MS (ESI) m/z (M+H)⁺ 473.1. ¹H NMR (Methanol-d₄, 400 MHz): δ 7.79 (d, J=7.6 Hz, 4H), 7.63-7.66 (m, 2H), 7.32-7.44 (m, 5H), 7.06 (d, J=8.8 Hz, 2H), 5.62 (s, 1H), 4.72 (s, 2H), 2.18 (s, 3H), 1.61 (d, J=5.6 Hz, 2H).

IT300 was prepared following the same procedure described in the synthesis of IT155 by Suzuki-Coupling of B9 and A1, followed by LiOH hydrolysis to afford the final product IT300.

IT302 was prepared following the same procedure described in the synthesis of IT155 by Suzuki-Coupling of intermediate 3-(4-bromo-2,6-difluorophenoxy)dihydrofuran-2(3H)-one and A1, followed by LiOH hydrolysis to afford the final product IT302.

IT304 was prepared following the same procedure described in the synthesis of IT155 by Suzuki-Coupling of E5 and A1, followed by LiOH hydrolysis to afford the final product IT304.

IT305 was prepared following the same procedure described in the synthesis of IT155 by Suzuki-Coupling of B10 and A1, followed by LiOH hydrolysis to afford the final product IT305.

IT316 was prepared following the same procedure described in the synthesis of IT155 by Suzuki-Coupling of intermediate methyl 2-(4-bromo-2,6-difluorophenoxy)benzoate and A1, followed by LiOH hydrolysis to afford the final product IT316.

IT407, IT408 and IT425 were prepared following the similar procedure described in the synthesis of IT316.

IT344 was prepared following the same procedure described in the synthesis of IT155 by Suzuki-Coupling of intermediate methyl 2-((6-bromopyridin-3-yl)oxy)benzoate and A1 using Pd(dtbpf)Cl₂ as catalyst, followed by LiOH hydrolysis to afford the final product IT344 as a white solid. MS (ESI) m/z (M+H)⁺ 536.1. ¹H NMR (Methanol-d₄, 400 MHz): δ 8.34 (d, J=8.0 Hz, 1H), 7.99 (d, J=8.0 Hz, 2H), 7.83-7.90 (q, J=7.2 Hz, 4H), 7.37-7.42 (t, J=8.0 Hz, 1H), 7.30-7.34 (m, 7H), 7.14 (d, J=8.0 Hz, 1H), 5.80-5.82 (m, 1H), 2.18 (s, 3H), 1.59-1.61 (d, J=5.6 Hz, 3H).

IT345 was prepared following the same procedure described in the synthesis of IT155 by Suzuki-Coupling of intermediate methyl 2-((5-bromopyridin-2-yl)oxy)benzoate and A1 using Pd(dtbpf)Cl₂ as catalyst, followed by LiOH hydrolysis to afford the final product IT345. MS (ESI) m/z (M+H)⁺ 536.1. ¹H NMR (Methanol-d₄, 400 MHz): δ 8.35 (s, 1H), 8.13 (d, J=8.0 Hz, 1H), 8.03 (d, J=8.0 Hz, 1H), 7.81 (d, J=8.0 Hz, 2H), 7.64-7.66 (m, 3H), 7.36-7.41 (m, 4H), 7.26 (d, J=8.0 Hz, 2H), 7.06 (d, J=8.0 Hz, 2H), 5.77-5.79 (br, 1H), 2.16 (s, 3H), 1.57-1.59 (d, J=6.0 Hz, 3H).

IT355 was prepared following the similar procedure described in the synthesis of IT155 by Suzuki-coupling of intermediate methyl 2-(4-bromo-2-fluorophenoxy)benzoate and A1, followed by LiOH hydrolysis to afford the final product IT355. ¹H NMR (400 MHz, DMSO-d₆): δ 13.01 (s, 1H), 9.33 (s, 1H), 7.86-7.88 (m, 8H), 7.78-7.80 (m, 2H), 7.40-7.45 (m, 3H), 7.30-7.32 (m, 2H), 7.08-7.10 (d, J=8.4 Hz, 1H), 6.96-7.01 (m, 1H), 5.73-5.75 (q, J=6.4 Hz, 1H), 2.10 (s, 3H), 1.52-1.54 (d, J=6.0 Hz, 3H). MS (ESI) m/z (M+H)⁺ 553.1.

IT356 was prepared following the similar procedure described in the synthesis of IT155 by Suzuki-coupling of intermediate methyl 3-(4-bromophenoxy)benzoate and A1, followed by LiOH hydrolysis to afford the final product IT356. ¹H NMR (400 MHz, DMSO-d₆): δ 9.31 (s, 1H), 7.71-7.78 (m, 8H), 7.48-7.55 (m, 2H), 7.36-7.40 (m, 5H), 7.16 (d, J=8.8 Hz, 2H), 5.73 (q, J=6.4 Hz, 1H), 2.01 (s, 3H), 1.52-1.53 (d, J=6.0 Hz, 3H). MS (ESI) m/z (M+H)⁺ 535.2.

IT368 was prepared following the similar procedure described in the synthesis of IT155 by Suzuki-coupling of intermediate methyl 4-(4-bromophenoxy)benzoate and A1, followed by LiOH hydrolysis to afford the final product IT368. ¹H NMR (400 MHz, DMSO-d₆): δ 9.33 (s, 1H), 7.95 (d, J=8.8 Hz, 2H), 7.78-7.80 (m, 7H), 7.40 (br, 3H), 7.30 (br, 1H), 7.21 (d, J=8.8 Hz, 2H), 7.09 (d, J=8.8 Hz, 2H), 5.72-5.74 (q, J=6.4 Hz, 1H), 2.01 (s, 3H), 1.52 (d, J=5.6 Hz, 3H). MS (ESI) m/z (M+H)⁺ 535.3.

IT374 was prepared following the similar procedure described in the synthesis of IT155 by Suzuki-coupling of intermediate methyl 2-(4-bromo-2-fluorophenoxy)-3-fluorobenzoate and A1, followed by LiOH hydrolysis to afford the final product IT374. ¹H NMR (DMSO-d₆, 400 MHz): δ 9.33 (s, 1H), 7.70-7.81 (m, 7H), 7.21-7.49 (m, 7H), 6.77 (t, J=8.8 Hz, 1H), 5.76 (d, J=6.4 Hz, 1H), 2.13 (s, 3H), 1.55 (d, J=5.6 Hz, 3H). MS (ESI) m/z (M+H)⁺ 571.2.

IT375 was prepared following the similar procedure described in the synthesis of IT155 by Suzuki-coupling of intermediate methyl 2-(4-bromophenoxy)-3-fluorobenzoate and A1, followed by LiOH hydrolysis to afford the final product IT375. ¹H NMR (DMSO-d₆, 400 MHz): δ 9.30 (s, 1H), 7.67-7.77 (m, 9H), 7.40-7.44 (m, 5H), 7.32 (s, 1H), 6.94 (d, J=8.4 Hz, 2H), 5.74 (d, J=6.8 Hz, 1H), 2.11 (s, 3H), 1.53 (d, J=6.8 Hz, 3H). MS (ESI) m/z (M+H)⁺ 553.2.

IT388 was prepared following the similar procedure described in the synthesis of IT155 by Suzuki-coupling of intermediate methyl 5-(4-bromophenoxy)furan-2-carboxylate and A1 using Pd(dtbpf)Cl₂ as catalyst, followed by LiOH hydrolysis to afford the final product IT388. MS (ESI) m/z (M+H)⁺ 525.1. ¹H NMR (Methanol-d₄ 400 MHz): δ 7.83-7.85 (m, 2H), 7.74-7.76 (m, 4H), 7.70-7.72 (m, 3H), 7.73 (m, 1H), 7.27-7.28 (m, 3H), 7.26 (m, 1H), 5.78 (m, 1H), 5.78 (s, 1H), 2.20 (s, 3H), 1.61-1.62 (m, 3H).

IT409 was prepared following the similar procedure described in the synthesis of IT155 by Suzuki-coupling of intermediate ethyl 5-(4-bromophenoxy)thiophene-2-carboxylate and A1 using Pd(dtbpf)Cl₂ as catalyst, followed by LiOH hydrolysis to afford the final product IT409. MS (ESI) m/z (M+H)⁺ 541.0. ¹H NMR (Methanol-d₄, 400 MHz): δ 7.69-7.84 (m, 6H), 7.59 (d, J=8.0 Hz, 1H), 7.29-7.44 (m, 7H), 6.59 (d, J=8.0 Hz, 1H), 5.81 (br, 1H), 2.19 (s, 3H), 1.60-1.62 (br, 3H).

IT417 was prepared following the similar procedure described in the synthesis of IT155 by Suzuki-coupling of intermediate ethyl 4-(4-bromophenoxy)thiazole-5-carboxylate and A1 using Pd(dtbpf)Cl₂ as catalyst, followed by LiOH hydrolysis to afford the final product IT417. MS (ESI) m/z (M+H)⁺ 542.0. ¹H NMR (Methanol-d₄ 400 MHz): δ 8.92 (s, 1H), 7.79-7.81 (m, 2H), 7.66-7.68 (m, 4H), 7.37-7.43 (m, 4H), 7.18-7.20 (m, 2H), 5.80-5.82 (m, 1H), 2.17 (s, 3H), 1.61-1.62 (d, J=6.4 Hz, 3H).

IT419 was prepared following the similar procedure described in the synthesis of IT155 by Suzuki-coupling of intermediate ethyl 4-(4-bromophenoxy)oxazole-5-carboxylate and A1 using Pd(dtbpf)Cl₂ as catalyst, followed by LiOH hydrolysis to afford the final product IT419 as a white solid. MS (ESI) m/z (M+H)⁺ 526.1. ¹H NMR (Methanol-d₄, 400 MHz): δ 8.21 (s, 1H), 7.68-7.80 (m, 6H), 7.24-7.43 (m, 7H), 5.80 (s, 1H), 2.18 (s, 3H), 1.60 (s, 3H).

IT420 was prepared following the similar procedure described in the synthesis of IT155 by Suzuki-coupling of intermediate ethyl 5-(4-bromophenoxy)thiazole-4-carboxylate and A1 using Pd(dtbpf)Cl₂ as catalyst, followed by LiOH hydrolysis to afford the final product IT420 as a white solid. MS (ESI) m/z (M+H)⁺ 542.1. ¹H NMR (Methanol-d4, 400 MHz): δ 8.59 (s, 1H), 7.82 (d, J=8.0 Hz, 2H), 7.69-7.76 (m, 5H), 7.39-7.44 (m, 3H), 7.31 (d, J=8.0 Hz, 3H), 5.81 (d, J=6.4 Hz, 1H), 2.18 (s, 3H), 1.61 (d, J=6.0 Hz, 3H).

IT428 was prepared following the similar procedure described in the synthesis of IT155 by Suzuki-coupling of intermediate methyl 3-(4-bromophenoxy)thiophene-2-carboxylate and A1 using Pd(dtbpf)Cl₂ as catalyst, followed by LiOH hydrolysis to afford the final product IT428. MS (ESI) m/z (M+H)⁺ 541.1. ¹H NMR (400 MHz, DMSO-d₆) δ 9.30 (s, 1H), 7.86-7.88 (d, J=5.2 Hz, 1H), 7.71-7.77 (m, 7H), 7.39-7.41 (m, 4H), 7.06-7.09 (d, J=8.8 Hz, 2H), 6.90-6.91 (d, J=5.2 Hz, 1H), 5.73-5.75 (d, J=6.4 Hz, 3H), 2.10 (s, 3H), 1.52-1.54 (d, J=6.0 Hz, 3H).

IT434 was prepared following the similar procedure described in the synthesis of IT155 by Suzuki-coupling of intermediate ethyl 5-(4-bromophenoxy)-2-ethyloxazole-4-carboxylate and A1 using Pd(dtbpf)Cl₂ as catalyst, followed by LiOH hydrolysis to afford the final product IT434 as a white solid. MS (ESI) m/z (M+H)⁺ 554.1. ¹H NMR (Methanol-d₄, 400 MHz): δ 7.70-7.82 (m, 6H), 7.22-7.43 (m, 7H), 5.81 (br, 1H), 2.74-2.80 (m, 2H), 2.18 (s, 3H), 1.61 (d, J=5.2 Hz, 3H), 1.30-1.34 (m, 3H).

IT435 was prepared following the similar procedure described in the synthesis of IT155 by Suzuki-coupling of intermediate methyl 3-(4-bromo-2,6-difluorophenoxy)picolinate and A1 using Pd(dtbpf)Cl₂ as catalyst, followed by LiOH hydrolysis to afford the final product IT435. MS (ESI) m/z (M+H)⁺ 572.1. ¹H NMR (Methanol-d₄, 400 MHz): δ 8.40 (m, 1H), 7.59-7.87 (m, 5H), 7.52-7.55 (m, 3H), 7.32-7.49 (m, 5H), 5.81 (br, 1H), 2.18 (s, 3H), 1.60-1.61 (br, 3H).

IT436 was prepared following the similar procedure described in the synthesis of IT155 by Suzuki-coupling of intermediate methyl 3-(4-bromo-2,6-difluorophenoxy)picolinate and A4 using Pd(dtbpf)Cl₂ as catalyst, followed by LiOH hydrolysis to afford the final product IT436. MS (ESI) m/z (M+H)⁺ 571.2. ¹H NMR (DMSO-d₆, 400 MHz): δ 9.79 (s, 1H), 8.30-8.36 (m, 2H), 7.34-7.87 (m, 12H), 6.02 (q, J=6.4 Hz, 1H), 3.64 (s, 3H), 1.57 (d, J=5.6 Hz, 3H).

IT437 was prepared following the similar procedure described in the synthesis of IT155 by Suzuki-coupling of intermediate methyl 3-(4-bromo-2,6-difluorophenoxy)picolinate and A5 using Pd(dtbpf)Cl₂ as catalyst, followed by LiOH hydrolysis to afford the final product IT437. MS (ESI) m/z (M+H)⁺ 572.2. ¹H NMR (Methanol-d₄, 400 MHz): δ 8.38 (d, J=4.0 Hz, 1H), 7.77 (d, J=6.4 Hz, 2H), 7.51-7.57 (m, 6H), 7.24-7.31 (brs, 5H), 5.70 (brs, 1H), 2.23 (s, 3H), 1.49 (brs, 3H).

IT438 was prepared following the similar procedure described in the synthesis of IT155 by Suzuki-coupling of intermediate methyl 4-(4-bromo-2,6-difluorophenoxy)nicotinate and A4 using Pd(dppf)Cl₂ as catalyst, followed by LiOH hydrolysis to afford the final product IT438. MS (ESI) m/z (M+H)⁺ 571.3. ¹H NMR (400 MHz, DMSO-d₆): δ 9.65 (s, 1H), 8.98 (s, 1H), 8.61 (d, J=5.6 Hz, 1H), 7.85 (s, 1H), 7.77-7.79 (m, 5H), 7.57-7.59 (d, J=7.6 Hz, 2H), 7.41 (br, 3H), 7.30 (br, 1H), 7.04-7.05 (d, J=5.2 Hz, 1H), 5.75 (q, J=6.4 Hz, 1H), 3.61 (s, 3H), 1.53-1.54 (d, J=6.4 Hz, 3H).

IT439 was prepared following the similar procedure described in the synthesis of IT155 by Suzuki-coupling of intermediate methyl 4-(4-bromo-2,6-difluorophenoxy)nicotinate and A2 using Pd(dppf)Cl₂ as catalyst, followed by LiOH hydrolysis to afford the final product IT439. MS (ESI) m/z (M+H)⁺ 588.2. ¹H NMR (400 MHz, DMSO-d₆): δ 9.32 (s, 1H), 8.92 (s, 1H), 8.57 (d, J=6.0 Hz, 1H), 7.88 (d, J=8.0 Hz, 2H), 7.82 (d, J=6.0 Hz, 2H), 7.62 (d, J=8.0 Hz, 2H), 7.19-7.39 (m, 5H), 6.95-6.96 (d, J=6.0 Hz, 1H), 5.71 (q, J=6.4 Hz, 1H), 2.24 (s, 3H), 1.50 (d, J=6.4 Hz, 3H).

IT440 was prepared following the similar procedure described in the synthesis of IT155 by Suzuki-coupling of intermediate methyl 4-(4-bromo-2,6-difluorophenoxy)nicotinate and A5 using Pd(dppf)Cl₂ as catalyst, followed by LiOH hydrolysis to afford the final product IT440. MS (ESI) m/z (M+H)⁺ 572.2. ¹H NMR (Methanol-d₄, 400 MHz): δ 9.02 (s, 1H), 8.55 (brs, 1H), 7.79 (m, 2H), 7.59 (m, 4H), 7.28 (brs, 5H), 6.95 (s, 1H), 5.70 (brs, 5H), 2.24 (s, 3H), 1.50 (brs, 3H).

IT444 was prepared following the similar procedure described in the synthesis of IT155 by Suzuki-coupling of intermediate ethyl 5-(4-bromophenoxy)-1-methyl-1H-imidazole-4-carboxylate and A1 using Pd(dtbpf)Cl₂ as catalyst, followed by LiOH hydrolysis to afford the final product IT444 as a yellow solid. ¹H NMR (Methanol-d₄, 400 MHz): δ 8.51 (br, 1H), 7.82 (br, 2H), 7.61-7.73 (m, 4H), 7.33-7.45 (br, 4H), 7.17-7.20 (m, 3H), 5.83 (m, 1H), 3.72 (s, 3H), 2.20 (s, 3H), 1.62 (br, 3H).

IT446 was prepared following the similar procedure described in the synthesis of IT155 by Suzuki-coupling of intermediate 2-(4-bromo-2-fluorophenoxy)benzoic acid and A4 using Pd(dtbpf)Cl₂ as catalyst to afford the final product IT446 as a white solid. MS (ESI) m/z (M+H)⁺552.3. ¹H NMR (DMSO-d₆, 400 MHz): δ 13.00 (br, 1H), 9.62 (br, 1H), 7.86 (dd, J₁=8.0 Hz, J₂=1.6 Hz, 1H), 7.81 (s, 1H), 7.26-7.73 (m, 13H), 7.04 (d, J=8.0 Hz, 1H), 6.97 (dd, J₁=8.4 Hz, J₂=8.4 Hz, 1H), 5.76 (br, 1H), 3.61 (s, 3H), 1.53 (d, J=6.4 Hz, 3H).

IT448 was prepared following the similar procedure described in the synthesis of IT155 by Suzuki-coupling of intermediate methyl 2-(2,6-difluoro-4-iodophenoxy)benzoate and A2 using Pd(dtbpf)Cl₂ as catalyst, followed by LiOH hydrolysis to afford the final product IT448. MS (ESI) m/z (M+H)⁺ 587.2. ¹H NMR (400 MHz, DMSO-d₆): δ 13.08 (s, 1H), 9.34 (s, 1H), 7.84-7.90 (m, 3H), 7.76-7.79 (d, J=9.6 Hz, 2H), 7.62 (d, J=8.0 Hz, 2H), 7.52-7.53 (m, 1H), 7.40 (br, 3H), 7.23 (br, 1H), 7.21-7.22 (m, 1H), 6.91 (d, J=8.8 Hz, 1H), 5.74 (q, J=6.8 Hz, 1H), 2.27 (s, 3H), 1.53 (d, J=6.0 Hz, 3H).

IT449 was prepared following the similar procedure described in the synthesis of IT155 by Suzuki-coupling of intermediate methyl 2-(2,6-difluoro-4-iodophenoxy)benzoate and chloro-substituted A1 (R)-1-(2-chlorophenyl)ethyl (3-methyl-5-(4-(4,4,5,5-tetramethyl-1,3,2-dioxaborolan-2-yl)phenyl)isoxazol-4-yl)carbamate using Pd(dtbpf)Cl₂ as catalyst, followed by LiOH hydrolysis to afford the final product IT449. MS (ESI) m/z (M+H)⁺ 605.1. ¹H NMR (400 MHz, DMSO-d₆): δ 13.09 (s, 1H), 9.49 (s, 1H), 7.87-7.94 (m, 2H), 7.85-7.86 (m, 5H), 7.83-7.84 (m, 1H), 7.78-7.81 (m, 3H), 7.49-7.51 (m, 1H), 6.90 (d, J=8.4 Hz, 1H), 5.99-6.02 (q, 1H), 2.13 (s, 3H), 1.55-1.57 (d, J=6.4 Hz, 3H).

IT450 was prepared following the similar procedure described in the synthesis of IT155 by Suzuki-coupling of intermediate methyl 2-(2,6-difluoro-4-iodophenoxy)benzoate and chloro-substituted A4 (R)-1-(2-chlorophenyl)ethyl (1-methyl-4-(4-(4,4,5,5-tetramethyl-1,3,2-dioxaborolan-2-yl)phenyl)-1H-pyrazol-5-yl)carbamate using Pd(dtbpf)Cl₂ as catalyst, followed by LiOH hydrolysis to afford the final product IT450. MS (ESI) m/z (M+H)⁺ 605.1. ¹H NMR (DMSO-d₆, 400 MHz): δ 8.49 (d, J=4.4 Hz, 1H), 7.74-7.82 (m, 3H), 7.35-7.64 (m, 11H), 5.84 (brs, 1H), 3.74 (s, 3H), 1.61 (brs, 3H).

IT451 was prepared following the similar procedure described in the synthesis of IT155 by Suzuki-coupling of intermediate methyl 2-(4-bromo-2,6-difluorophenoxy)nicotinate and A2 using Pd(dtbpf)Cl₂ as catalyst, followed by LiOH hydrolysis to afford the final product IT451. MS (ESI) m/z (M+H)⁺ 588.2. ¹H NMR (400 MHz, DMSO-d₆): δ 13.49 (s, 1H), 9.34 (s, 1H), 8.31-8.37 (m, 2H), 7.89 (d, J=7.6 Hz, 2H), 7.65-7.74 (m, 4H), 7.03-7.41 (m, 6H), 5.74 (q, J=6.8 Hz, 1H), 2.27 (s, 3H), 1.53 (d, J=6.8 Hz, 3H).

IT453 was prepared following the similar procedure described in the synthesis of IT155 by Suzuki-coupling of methyl 4-((3,5-difluoro-4′-(4,4,5,5-tetramethyl-1,3,2-dioxaborolan-2-yl)-[1,1′-biphenyl]-4-yl)oxy)nicotinate with (R)-1-phenylethyl (4-iodo-1-methyl-1H-1,2,3-triazol-5-yl)carbamate using Pd(dtbpf)Cl₂ as catalyst, followed by LiOH hydrolysis to afford the final product IT453. MS (ESI) m/z (M+H)⁺ 572.2. ¹H NMR (400 MHz, DMSO-d₆): δ 10.02 (s, 1H), 8.98 (s, 1H), 8.62 (d, J=5.6 Hz, 1H), 7.81-7.85 (m, 6H), 7.04-7.43 (m, 6H), 5.78 (brs, 1H), 3.85 (s, 3H), 1.56 (brs, 3H).

IT454 was prepared by standard Suzuki-coupling of A1 and B11 and conversion of the corresponding ester to acid to afford IT454 as a white solid. IT455-IT457 were prepared from the similar procedure described in the synthesis of IT454.

IT459 was prepared by standard Suzuki-coupoing of A1 and B12 and conversion of the corresponding ester to acid to afford IT459 as a white solid. IT460 and IT461 were prepared from the similar procedure described in the synthesis of IT459.

IT462 was prepared following the similar procedure described in the synthesis of IT155 by Suzuki-coupling of intermediate methyl 3-(2,6-difluoro-4-iodophenoxy)thiophene-2-carboxylate with A2 using Pd(dtbpf)Cl₂ as catalyst, followed by LiOH hydrolysis to afford the final product IT462. MS (ESI) m/z (M+H)⁺ 593.1. ¹H NMR (Methanol-d₄, 400 MHz): δ 7.58-7.67 (m, 5H), 7.19-7.46 (m, 7H), 6.64 (d, J=5.2 Hz, 1H), 5.76 (d, J=5.6 Hz, 1H), 2.32 (s, 3H), 1.56 (d, J=5.6 Hz, 3H).

IT463 was prepared following the similar procedure described in the synthesis of IT155 by Suzuki-coupling of intermediate methyl 3-(2,6-difluoro-4-iodophenoxy)thiophene-2-carboxylate with A1 using Pd(dtbpf)Cl₂ as catalyst, followed by LiOH hydrolysis to afford the final product IT463 as a white solid. MS (ESI) m/z (M+H)⁺ 577.0. ¹H NMR (Methol-d₄, 400 MHz): δ 7.84-7.86 (m, 2H), 7.72-7.74 (m, 2H), 7.59 (d, J=5.6 Hz, 1H), 7.32-7.39 (m, 7H), 6.65 (d, J=5.6 Hz, 1H), 5.81 (br, 1H), 2.18 (s, 3H), 1.61 (d, J=5.2 Hz, 3H).

IT464 was prepared following the similar procedure described in the synthesis of IT155 by Suzuki-coupling of intermediate methyl 3-(4-bromo-2,6-difluorophenoxy)picolinate and A2 using Pd(dtbpf)Cl₂ as catalyst, followed by LiOH hydrolysis to afford the final product IT464 as a white solid. ¹H NMR (Methanol-d₄, 400 Hz): δ 8.40 (s, 1H), 7.29-7.70 (m, 13H), 5.76 (m, 1H), 2.33 (s, 3H), 1.56 (br, 3H). MS (ESI) m/z (M+H)⁺ 588.1.

IT465 was prepared following the similar procedure described in the synthesis of IT155 by Suzuki-coupling of intermediate methyl 3-(2,6-difluoro-4-iodophenoxy)thiophene-2-carboxylate and A4 using Pd(dtbpf)Cl₂ as catalyst, followed by LiOH hydrolysis to afford the final product IT465. MS (ESI) m/z (M+H)⁺ 576.0. ¹H NMR (Methanol-d₄, 400 MHz): δ 7.34-7.79 (m, 13H), 6.66 (d, J=5.6 Hz, 1H), 5.84 (s, 2H), 3.74 (s, 3H), 1.62 (s, 3H).

IT467 was prepared by standard Suzuki-coupoing of A1 and B13 and conversion of the corresponding ester to acid to afford IT467 as a white solid. IT468 and IT469 were prepared from the similar procedure described in the synthesis of IT467.

IT471 was prepared following the similar procedure described in the synthesis of IT155 by Suzuki-coupling of intermediate ethyl 4-(4-bromophenoxy)-1-methyl-1H-1,2,3-triazole-5-carboxylate and A1 using Pd(dtbpf)Cl₂ as catalyst, followed by LiOH hydrolysis to afford the final product IT471 as a white solid. MS (ESI) m/z (M+H)⁺ 540.1. ¹H NMR (DMSO-d₆, 400 MHz): δ 9.29 (s, 1H), 7.71-7.78 (m, 6H), 7.31-7.41 (m, 4H), 7.15-7.17 (m, 3H), 5.73-5.75 (m, 1H), 4.23 (s, 3H), 2.11 (s, 3H), 1.53-1.54 (d, J=5.6 Hz, 3H).

IT486 was prepared following the similar procedure described in the synthesis of IT444 by Suzuki-coupling of ethyl 4-(4-bromophenoxy)-1-methyl-1H-imidazole-5-carboxylate and A1, followed by LiOH hydrolysis to afford the final product as a white solid. MS (ESI) m/z (M+H)⁺539.1. ¹H NMR (Methanol-d₄, 400 MHz): δ 7.79 (d, J=7.6 Hz, 2H), 7.63-7.67 (m, 5H), 7.33-7.43 (m, 4H), 7.10-7.31 (m, 3H), 5.81 (br, 1H), 3.93 (s, 3H), 2.18 (s, 3H), 1.60 (d, J=5.6 Hz, 3H).

IT490 was prepared following the similar procedure described in the synthesis of IT155 by Suzuki-coupling of B14 and A1 using Pd(dtbpf)Cl₂ as catalyst, followed by LiOH hydrolysis to afford the final product. MS (ESI) m/z (M+H)⁺ 627.0. ¹H NMR (Methanol-d₄, 400 MHz): δ 8.38-8.40 (d, J=8.0 Hz, 1H), 7.87 (s, 2H), 7.78 (s, 2H), 7.67-7.69 (d, J=8.0 Hz, 1H), 7.56-7.58 (d, J=9.2 Hz, 2H), 7.41-7.43 (m, 4H), 7.30-7.32 (m, 2H), 7.19 (s, 1H), 5.80 (s. 1H), 2.19 (s, 3H), 1.60-1.62 (d, J=5.2 Hz, 3H).

IT491 was prepared following the similar procedure described in the synthesis of IT155 by Suzuki-coupling of E5 and A6 using Pd(dtbpf)Cl₂ as catalyst, followed by LiOH hydrolysis to afford the final product. ¹H NMR (Methanol-d₄, 400 MHz): δ 7.93-7.94 (d, J=7.2 Hz, 2H), 7.64 (s, 2H), 7.42-7.50 (m, 5H), 7.24-7.33 (m, 6H), 5.84 (s, 1H), 1.65 (s, 3H), 1.53-1.57 (m, 2H), 1.38-1.41 (m, 2H).

IT492 was prepared following the similar procedure described in the synthesis of IT491 using methyl 1-(4-bromo-2-fluorophenoxy)cyclopropanecarboxylate (E6) in place of E5. MS (ESI) m/z (M−H)⁺ 550.5. ¹H NMR (Methanol-d₄, 400 MHz): δ 7.93-7.95 (m, 2H), 7.64-7.65 (m, 2H), 7.78 (s, 2H), 7.51 (m, 7H), 7.48 (m, 2H), 7.45 (m, 2H), 7.43 (m, 2H), 5.87 (brs, 1H), 1.66-4.69 (m, 5H), 1.39-1.42 (m, 2H).

IT497 was prepared following the similar procedure described in the synthesis of IT155 by Suzuki-coupling of B15 and A3 using Pd(dtbpf)Cl₂ as catalyst, followed by LiOH hydrolysis to afford the final product. Sodium salt IT497a: ¹H NMR (DMSO-d₆, 400 MHz): δ 10.14 (s, 1H), 7.74-7.76 (m, 2H), 7.59-7.63 (m, 5H), 7.34-7.36 (m, 5H), 6.91-6.93 (m, 3H), 6.70-6.72 (m, 1H), 5.75 (d, J=6.4 Hz, 1H), 3.82 (s, 3H), 1.50 (br, 3H). MS (ESI) m/z (M+H)⁺ 553.3. IT514 was prepared following the similar procedure described in the synthesis of IT497.

IT500 was prepared following the similar procedure described in the synthesis of IT155 by Suzuki-coupling of E6 and A1 using Pd(dtbpf)Cl₂ as catalyst, followed by LiOH hydrolysis to afford the final product as a white solid. ¹H NMR (Methanol-d₄, 400 MHz): δ 7.83 (br, 2H), 7.69 (br, 2H), 7.08-7.43 (m, 7H), 2.18 (s, 3H), 1.58 (s, 9H).

IT501 was prepared following the similar procedure described in the synthesis of IT155 by Suzuki-coupling of E6 and A6 using Pd(dtbpf)Cl₂ as catalyst, followed by LiOH hydrolysis to afford the final product as a white solid. ¹H NMR (Methanol-d₄, 400 MHz): δ 7.97 (br, 2H), 7.69 (br, 2H), 7.53-7.27 (m, 11H), 5.87 (m, 1H), 1.66 (s, 3H), 1.60 (s, 6H).

IT502 was prepared following the similar procedure described in the synthesis of IT155 by Suzuki-coupling of E6 and A2 using Pd(dtbpf)Cl₂ as catalyst, followed by LiOH hydrolysis to afford the final product as a white solid. ¹H NMR (Methanol-d₄, 400 Hz): δ 7.55-7.65 (m, 4H), 7.28-7.36 (m, 7H), 5.78 (m, 1H), 2.34 (s, 3H), 1.58 (br, 9H).

IT503 was prepared following the similar procedure described in the synthesis of IT155 by Suzuki-coupling of E6 and A4 using Pd(dtbpf)Cl₂ as catalyst, followed by LiOH hydrolysis to afford the final product as a white solid. ¹H NMR (Methanol-d₄, 400 MHz): δ 7.75 (s, 1H), 7.17-7.52 (m, 11H), 5.82 (m, 1H), 3.71 (s, 3H), 1.58 (br, 9H).

IT504 was prepared following the similar procedure described in the synthesis of IT155 by Suzuki-coupling of E7 and A6 using Pd(dtbpf)Cl₂ as catalyst, followed by LiOH hydrolysis to afford the final product as a white solid. ¹H NMR (Methanol-d₄, 400 MHz): δ 7.90 (d, J=8.0 Hz, 2H), 7.16-7.61 (m, 14H), 5.85 (br, 1H), 1.60-1.76 (m, 9H).

IT505 was prepared following the similar procedure described in the synthesis of IT155 by Suzuki-coupling of E7 and A4 using Pd(dtbpf)Cl₂ as catalyst, followed by LiOH hydrolysis to afford the final product as a white solid. MS (ESI) m/z (M+H)⁺ 518.3. ¹H NMR (Methanol-d₄, 400 MHz): δ 7.74 (s, 1H), 7.34-7.51 (m, 10H), 7.16-7.32 (m, 2H), 5.81 (br, 1H), 3.70 (s, 3H), 1.54-1.59 (m, 9H).

IT506 was prepared following the similar procedure described in the synthesis of IT155 by Suzuki-coupling of E7 and A1 using Pd(dtbpf)Cl₂ as catalyst, followed by LiOH hydrolysis to afford the final product as a white solid. MS (ESI) m/z (M+H)⁺ 519.2. ¹H NMR (Methanol-d₄, 400 MHz): δ 7.81 (d, J=8.0 Hz, 2H), 7.67 (d, J=7.6 Hz, 2H), 7.09-7.48 (m, 8H), 5.81 (d, J=6.4 Hz, 1H), 2.18 (s, 3H), 1.61 (s, 9H).

IT508 was prepared following the similar procedure described in the synthesis of IT155 by Suzuki-coupling of E3 and A6 using Pd(dtbpf)Cl₂ as catalyst, followed by LiOH hydrolysis to afford the final product. ¹H NMR (Methanol-d₄, 400 MHz): δ 7.91 (d, J=7.2 Hz, 2H), 7.23-7.63 (m, 13H), 7.00 (d, J=8.4 Hz, 2H), 5.85 (s, 1H), 1.62 (s, 9H).

IT510 was prepared following the similar procedure described in the synthesis of IT155 by Suzuki-coupling of E3 and A4 using Pd(dtbpf)Cl₂ as catalyst, followed by LiOH hydrolysis to afford the final product. MS (ESI) m/z (M+H)⁺ 500.2. ¹H NMR (Methanol-d₄, 400 MHz): δ 7.73 (s, 1H), 7.16-7.53 (m, 11H), 6.98 (d, J=8.2 Hz, 2H), 5.82 (s, 1H), 3.71 (s, 3H), 1.61 (s, 9H).

IT511 was prepared following the similar procedure described in the synthesis of IT155 by Suzuki-coupling of E1 and A4 using Pd(dtbpf)Cl₂ as catalyst, followed by LiOH hydrolysis to afford the final product. ¹H NMR (Methanol-d₄, 400 MHz): δ 7.74 (s, 1H), 7.17-7.56 (m, 11H), 7.02-7.05 (d, J=8.8 Hz, 2H), 5.84 (brs. 1H), 3.71 (s, 3H), 1.63-1.66 (m, 5H), 1.32-1.35 (m, 2H).

IT512 was prepared following the similar procedure described in the synthesis of IT155 by Suzuki-coupling of E1 and A6 using Pd(dtbpf)Cl₂ as catalyst, followed by LiOH hydrolysis to afford the final product. ¹H NMR (Methanol-d₄, 400 MHz): δ 7.91-7.93 (d, J=6.8 Hz, 2H), 7.60-7.62 (m, 4H), 7.50 (m, 3H), 7.43 (m, 5H), 7.33-7.35 (d, J=8.0 Hz, 1H), 7.06-7.05 (m, 2H), 5.87 (s. 1H), 1.66 (m, 5H), 1.35 (m, 2H).

IT513 was prepared following the similar procedure described in the synthesis of IT155 by Suzuki-coupling of E1 and A2 using Pd(dtbpf)Cl₂ as catalyst, followed by LiOH hydrolysis to afford the final product. MS (ESI) m/z (M+H)⁺ 515.1. ¹H NMR (Methanol-d₄, 400 MHz): δ 7.50-7.61 (m, 6H), 7.28-7.38 (m, 4H), 7.04-7.06 (m, 3H), 5.77 (br, 1H), 2.32 (s, 3H), 1.62-1.65 (m, 2H), 1.56 (d, J=5.2 Hz, 3H), 1.31-1.34 (m, 2H)

### Synthesis of Compound IT303

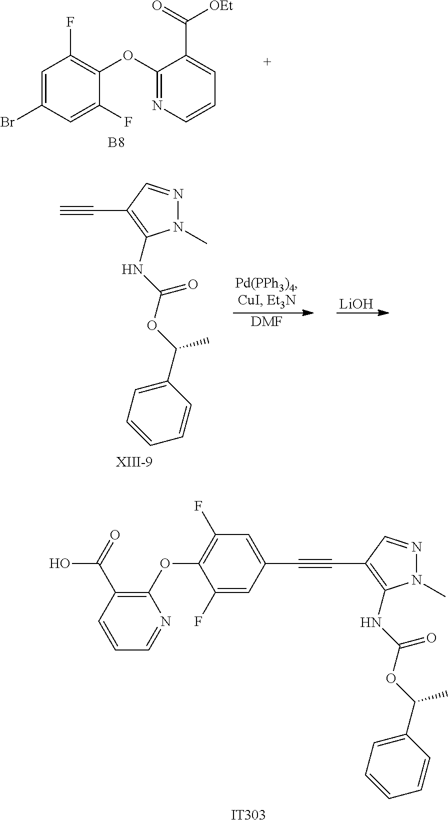

The preparation of XIII-9 was discussed in details in Example 6-B.

Ethyl 2-(4-bromo-2,6-difluorophenoxy)nicotinate (B8) (165 mg, 0.46 mmol) and (R)-1-phenylethyl (4-ethynyl-1-methyl-1H-pyrazol-5-yl)carbamate (XIII-9) (125 mg, 0.46 mmol) were dissolved in 4 mL dry DMF. To the solution was added CuI (90 mg, 0.46 mmol), Pd(PPh₃)₄ (106 mg, 0.09 mmol) and 0.4 mL Et₃N. The resulting mixture was heated at 85° C. in a sealed-tube overnight. The mixture was diluted with 20 mL EA and 10 mL hexane, washed with water and brine. The crude mixture was purified on ISCO to the intermediate ester which was subject to LiOH hydrolysis to afford IT303 as a white solid.

IT311 was prepared following the similar procedure described in the synthesis of IT303 using methyl 1-(4-bromo-2,6-difluorophenoxy)cyclopropanecarboxylate (E5) in place of B8.

### Synthesis of Compound IT306

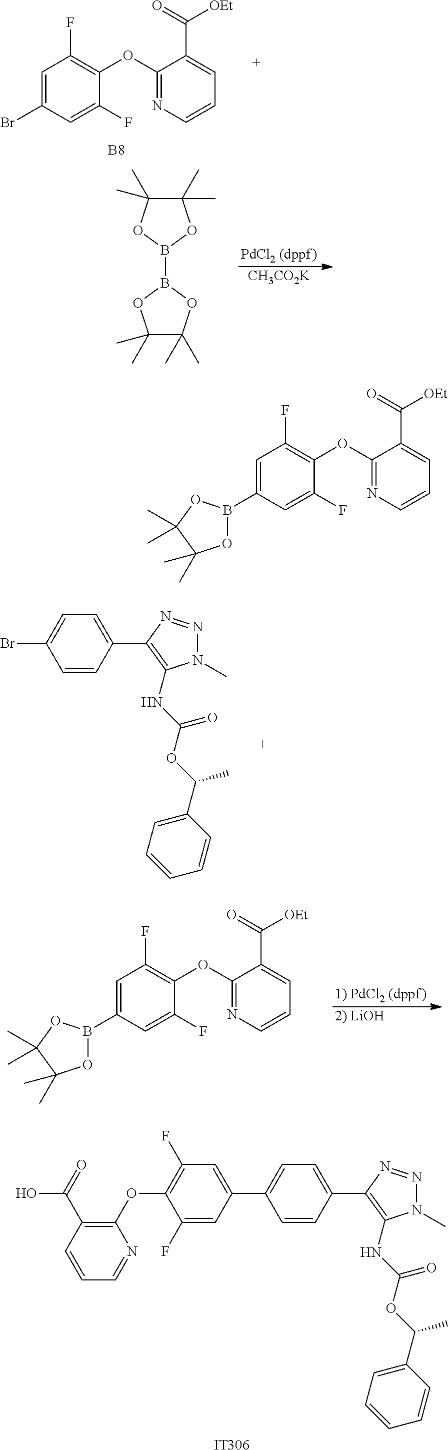

Ethyl 2-(4-bromo-2,6-difluorophenoxy)nicotinate (B8) (370 mg, 1.03 mmol), 4,4,4′,4′,5,5,5′,5′-octamethyl-2,2′-bi(1,3,2-dioxaborolane) (315 mg, 1.24 mmol) and potassium acetate (132 mg, 1.34 mmol) were combined in 8 mL dry 1,4-dioxane. The mixture was flushed with N₂, then added PdCl₂(dppf)CH₂Cl₂ (150 mg, 0.21 mmol). The resulting mixture was heated at 100° C. for 3 h in a sealed-tube. The reaction mixture was diluted with 30 mL EA. The precipitated solid was removed by filtration. The solvent was concentrated in vacuum to afford dark oil, which was directly purified on ISCO silica gel column to provide ethyl 2-(2,6-difluoro-4-(4,4,5,5-tetramethyl-1,3,2-dioxaborolan-2-yl)phenoxy)nicotinate (280 mg, 0.69 mmol) as an oil, which was subject to standard Pd(dppf)Cl₂ catalyzed Suzuki-coupling with (R)-1-phenylethyl (4-(4-bromophenyl)-1-methyl-1H-1,2,3-triazol-5-yl)carbamate and subsequent LiOH hydrolysis to afford IT306 as the final product.

IT307 and IT308 were prepared following the similar procedure described in the synthesis of IT306 using the corresponding (R)-1-phenylethyl (1-(4-bromophenyl)-4-methyl-1H-1,2,3-triazol-5-yl)carbamate and (R)-1-phenylethyl (4-(4-bromophenyl)-1-methyl-1H-pyrazol-5-yl)carbamate respectively.

IT309 was prepared following the similar procedure described in the synthesis of IT306 by Suzuki-coupling of methyl 2-(4-(4,4,5,5-tetramethyl-1,3,2-dioxaborolan-2-yl)phenoxy)benzoate and (R)-1-phenylethyl (4-(4-bromophenyl)-1-methyl-1H-1,2,3-triazol-5-yl)carbamate. Sodium salt IT309a: ¹H NMR (400 MHz, DMSO-d₆): δ 7.77 (d, J=8.4 Hz, 1H), 7.55-7.59 (m, 4H), 7.50 (dd, J₁=7.6 Hz, J₂=1.6 Hz, 1H), 7.19-7.33 (m, 6H), 7.063-7.10 (m, 1H), 6.86-6.89 (m, 3H), 5.73 (q, J=6.4 Hz, 1H), 3.77 (s, 3H), 1.45 (d, J=6.4, 3H). MS (ESI) m/z (M+H)⁺ 535.3.

IT447 was prepared following the similar procedure described in the synthesis of IT306 using the corresponding methyl 2-(2-fluoro-4-(4,4,5,5-tetramethyl-1,3,2-dioxaborolan-2-yl)phenoxy)benzoate. ¹H NMR (Methanol-d₄, 400 MHz): δ 13.0 (brs, 1H), 9.96 (brs, 1H), 7.85-7.87 (m, 1H), 7.76-7.78 (m, 5H), 7.73 (m, 1H), 7.29-7.58 (m, 7H), 7.05-7.07 (m, 1H), 6.97-6.98 (m, 1H), 5.76-5.77 (m, 1H), 3.83 (s, 3H), 1.55 (brs, 3H). MS (ESI) m/z (M+H)⁺ 553.3.

IT452 was prepared following the similar procedure described in the synthesis of IT306 using the corresponding methyl 3-(2,6-difluoro-4-(4,4,5,5-tetramethyl-1,3,2-dioxaborolan-2-yl)phenoxy)picolinate. ¹H NMR (DMSO-d₆, 400 MHz): δ 8.50 (m, 1H), 7.83-7.86 (m, 2H), 7.72-7.77 (m, 4H), 7.55-7.58 (m, 2H), 7.30-7.46 (m, 5H), 5.85 (m, 1H), 3.94 (s, 3H), 1.65 (br, 3H). MS (ESI) m/z (M+H)⁺ 572.2.

IT474 was prepared following the similar procedure described in the synthesis of IT306 using the corresponding difluoro substituted carbamate (R)-1-phenylethyl (5-(4-bromo-2,5-difluorophenyl)-3-methylisoxazol-4-yl)carbamate as a white solid. ¹H NMR (DMSO-d₆, 400 MHz): δ 7.84-7.85 (m, 1H), 7.79-7.81 (m, 1H), 7.65-7.73 (m, 2H), 7.51-7.53 (m, 2H), 7.26-7.35 (m, 4H), 7.00-7.03 (m, 1H), 5.68-5.69 (m, 1H), 2.16 (s, 3H), 1.48 (br 3H). MS (ESI) m/z (M+H)⁺ 608.1.

IT507 was prepared following the similar procedure described in the synthesis of IT306 by four Suzuki-coupling reactions: (1) E7 and bis(pinacolato)diboron to form an intermediate; (2) subsequent coupling with 1-bromo-4-iodobenzene to from a second intermediate; (3) coupling with bis(pinacolato)diboron again to form a third intermediate ethyl 2-((3-fluoro-4′-(4,4,5,5-tetramethyl-1,3,2-dioxaborolan-2-yl)-[1,1′-biphenyl]-4-yl)oxy)-2-methylpropanoate; (4) final coupling reaction with (R)-1-phenylethyl (5-iodo-3-methylisothiazol-4-yl)carbamate using Pd(dtbpf)Cl₂ as catalyst, followed by LiOH hydrolysis to afford the final product. MS (ESI) m/z (M+H)⁺ 535.1. ¹H NMR (Methanol-d₄, 400 MHz): δ 7.54-7.64 (m, 4H), 7.36-7.48 (m, 6H), 7.19-7.22 (m, 2H), 5.78 (br, 1H), 2.34 (s, 3H), 1.63 (s, 6H), 1.57-1.59 (d, J=6.4 Hz, 3H).

IT509 was prepared following the similar procedure described in the synthesis of IT507 using E3 as starting material. MS (ESI) m/z (M+H)⁺ 517.1. ¹H NMR (Methanol-d₄, 400 MHz): δ 7.50-7.61 (m, 6H), 7.34-7.38 (m, 4H), 7.27 (s, 1H), 7.00 (d, J=8.8 Hz, 2H), 5.77 (br, 1H), 2.32 (s, 3H), 1.62 s, 6H), 1.56 (d, J=6.4 Hz, 3H).

### Synthesis of Compound IT406

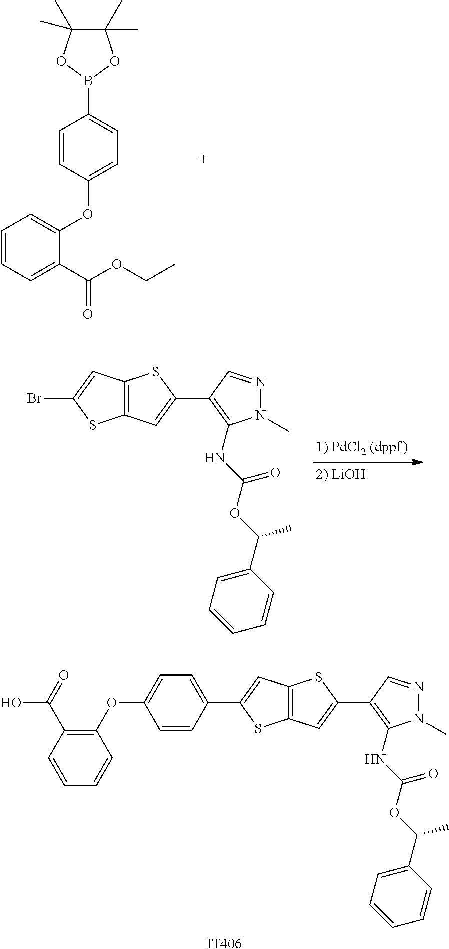 [SMILES: CCOC(=O)c1ccccc1Oc1ccc(B2OC(C)(C)C(C)(C)O2)cc1 ; C[C@@H](OC(=O)Nc1c(-c2cc3sc(Br)cc3s2)cnn1C)c1ccccc1 ; C[C@@H](OC(=O)Nc1c(-c2cc3sc(-c4ccc(Oc5ccccc5C(=O)O)cc4)cc3s2)cnn1C)c1ccccc1 ; "IT406"]

(R)-1-phenylethyl (4-(5-bromothieno[3,2-b]thiophen-2-yl)-1-methyl-1H-pyrazol-5-yl)carbamate (80 mg, 0.17 mmol) and ethyl 2-(4-(4,4,5,5-tetramethyl-1,3,2-dioxaborolan-2-yl)phenoxy)benzoate (62 mg, 0.17 mmol) were dissolved in 2 mL 1,4-dioxane, then added 2M K₂CO₃ in water (0.2 mL). The mixture was flushed with N₂, then added PdCl₂(dppf)CH₂Cl₂ (26 mg, 0.034 mmol). The resulting mixture was heated at 90° C. for 2 hrs in a sealed-tube. The reaction mixture was diluted with 40 mL EA, washed with water and brine. The solvent was concentrated in vacuum to afford dark oil, which was directly purified to provide the ester intermediate as a white solid (48 mg, yield 45%), which was dissolved in MeOH and THF and subjected to hydrolysis by 2N LiOH to afford IT406 (38 mg, 0.064 mmol) as a white solid.

IT470 was prepared following the similar procedure described in the synthesis of IT406 using the corresponding ethyl 2-(2,6-difluoro-4-(4,4,5,5-tetramethyl-1,3,2-dioxaborolan-2-yl)phenoxy)nicotinate and Pd(dtbpf)Cl₂ as catalyst. Sodium salt IT470a: ¹H NMR (DMSO-d₆, 400 MHz): δ 7.88-7.93 (m, 3H), 7.71 (s, 1H), 7.55 (d, J=8.8 Hz, 2H), 7.31-7.38 (m, 6H), 7.06-7.07 (m, 1H), 5.78 (q, J=6.4 Hz, 1H), 3.56 (s, 3H), 1.49 (d, J=6.4 Hz, 1H) MS (ESI) m/z (M+H)⁺ 633.2.

IT488 was prepared following the similar procedure described in the synthesis of IT470 by Suzuki-coupling of ethyl 1-(2,6-difluoro-4-(4,4,5,5-tetramethyl-1,3,2-dioxaborolan-2-yl)phenoxy)cyclopropanecarboxylate and (R)-1-phenylethyl (5-(5-bromothieno[3,2-b]thiophen-2-yl)-3-methylisoxazol-4-yl)carbamate, followed by LiOH hydrolysis to afford the final product. MS (ESI) m/z (M+H)⁺ 597.2. ¹H NMR (Methanol-d₄, 400 MHz): δ 7.63-7.67 (m, 2H), 7.36-7.47 (m, 6H), 7.16 (s, 1H), 5.84-5.86 (m. 1H), 2.19 (s, 3H), 1.64-1.65 (d, J=6.0 Hz, 3H), 1.55-1.59 (m, 2H), 1.42-1.44 (m, 2H). Sodium salt IT488a: MS (ESI) m/z (M+H)⁺ 597.1. ¹H NMR (Methanol-d₄, 400 MHz): δ 7.65 (s, 2H), 7.33-7.48 (m, 4H), 7.27-7.29 (m, 2H), 7.15 (s, 1H), 5.84-5.86 (m. 1H), 2.19 (s, 3H), 1.65 (d, J=6.4 Hz, 3H), 1.44-1.47 (m, 2H), 1.13-1.16 (m, 2H).

Additional compounds of Formula (I) through Formula (XVI) were prepared and characterized as shown in Table 14 according to those synthetic schemes described herein.

TABLE 14

| | MS | |
|---|---|---|
| | [m/z | |
| Compound # | (M + H)⁺] | NMR |
| IT123 | 493.1 | ¹H NMR (400 MHz, DMSO-d6) δ: 9.37 (s, 1H), 7.81-7.89 (m, 6H), 7.67 (d, |
| | | 2H), 7.06-7.40 (m, 5H), 5.74 (q, 1H), 2.11 (s, 3H), 1.53 (d, 3H) |
| IT124 | 477.1 | ¹H NMR (400 MHz, DMSO-d6) δ: 9.38 (s, 1H), 8.01 (m, 4H), 7.86 (m, 4H), |
| | | 7.05-7.41 (m, 5H), 5.72 (q, 1H), 3.26 (s, 3H), 2.11 (s, 3H), 1.53 (d, 3H) |
| IT128 | 492.1 | ¹H NMR (400 MHz, DMSO-d6) δ: 9.37 (s, 1H), 7.85-7.96 (m, 8H), |
| | | 7.06-7.57 (m, 5H), 5.74 (q, 1H), 2.43 (s, 3H), 2.11 (s, 3H), 1.53 (d, 3H) |
| IT129 | 478.0 | ¹H NMR (400 MHz, DMSO-d6) δ: 9.37 (s, 1H), 7.84-7.94 (m, 8H), |
| | | 7.06-7.43 (m, 5H), 5.74 (q, 1H), 2.11 (s, 3H), 1.53 (d, 3H) |
| IT130 | 562.2 | |
| IT131 | 520.1 | ¹H NMR (400 MHz, DMSO-d6) δ: 9.37 (s, 1H), 7.85-8.00 (m, 8H), |
| | | 7.04-7.41 (m, 5H), 5.74 (q, 1H), 2.11 (s, 3H), 1.92 (s, 3H), 1.53 (d, 3H) |
| IT136 | 533.2 | ¹H NMR (400 MHz, DMSO-d6) δ: 9.33 (s, 1H), 7.66-7.77 (m, 6H), |
| | | 7.05-7.48 (m, 7H), 5.73 (q, 1H), 3.01 (m, 4H), 2.10 (s, 3H), 1.53 (d, 3H) |
| IT137 | 465.1 | ¹H NMR (400 MHz, DMSO-d6) δ: 9.38 (s, 1H), 7.68-8.08 (m, 8H), |
| | | 7.04-7.40 (m, 5H), 5.73 (q, 1H), 2.10 (s, 3H), 1.92 (s, 3H), 1.53 (d, 3H) |
| IT138 | 546.2 | ¹H NMR (400 MHz, DMSO-d6) δ: 9.37 (s, 1H), 7.84-7.99 (m, 8H), |
| | | 7.05-7.41 (m, 5H), 5.74 (q, 1H), 2.11 (s, 3H), 1.67 (m, 1H), 1.92 (s, 3H), 1.53 (d, 3H), |
| | | 0.79 (m, 2H), 0.68 (m, 2H) |
| IT139 | 478.1 | ¹H NMR (400 MHz, DMSO-d6) δ: 9.40 (s, 1H), 9.15 (d, 1H), 8.48 (dd, 1H), |
| | | 8.14 (d, 1H), 7.87-7.98 (m, 4H), 7.06-7.41 (m, 5H), 5.73 (q, 1H), 3.26 (s, 3H), |
| | | 2.12 (s, 3H), 1.53 (d, 3H) |
| IT153 | 532.2 | ¹H NMR (400 MHz, DMSO-d6) δ: 9.38 (s, 1H), 7.83-7.98 (m, 8H), |
| | | 7.06-7.41 (m, 5H), 5.73 (q, 1H), 3.17 (m, 4H), 2.11 (s, 3H), 1.66 (m, 4H), 1.53 (d, 3H) |
| IT154 | 467.2 | ¹H NMR (400 MHz, DMSO-d6) δ: 9.37 (s, 1H), 7.05-7.86 (m, 13H), 5.73 (q, |
| | | 1H), 2.10 (s, 3H), 1.53 (d, 3H) |
| IT155 | 535.2 | ¹H NMR (400 MHz, DMSO-d6) δ: 9.32 (s, 1H), 7.66-7.77 (m, 7H), |
| | | 7.18-7.50 (m, 7H), 7.04 (d, 1H), 6.95 (d, 2H), 5.73 (q, 1H), 2.09 (s, 3H), 1.53 (d, 3H) |
| IT172 | 519.2 | ¹H NMR (400 MHz, DMSO-d6) δ: 9.35 (s, 1H), 7.68-7.85 (m, 6H), |
| | | 7.05-7.51 (m, 7H), 5.73 (q, 1H), 2.22-2.61 (m, 2H), 2.11 (s, 3H), 1.53 (d, 3H) |
| IT173 | 503.2 | ¹H NMR (400 MHz, DMSO-d6) δ: 9.38 (s, 1H), 7.78-8.00 (m, 8H), |
| | | 7.05-7.41 (m, 5H), 5.73 (q, 1H), 2.88 (m, 1H), 2.11 (s, 3H), 1.53 (d, 3H), 1.14 (m, 2H), |
| | | 1.05 (m, 2H) |
| IT174 | 517.2 | ¹H NMR (400 MHz, DMSO-d6) δ: 9.28 (s, 1H), 7.73-7.91 (m, 8H), |
| | | 6.94-7.30 (m, 5H), 5.63 (q, 1H), 3.23 (m, 2H), 2.02 (s, 3H), 1.43 (d, 3H), 0.76 (m, 1H), |
| | | 0.35 (m, 2H), 0.02 (m, 2H) |
| IT194 | 514.3 | ¹H NMR (400 MHz, DMSO-d6) δ: 9.51 (s, 1H), 9.30 (s, 1H), 7.54-7.74 (m, |
| | | 8H), 7.05-7.40 (m, 5H), 5.73 (q, 1H), 2.10 (s, 3H), 1.52 (d, 3H), 1.47 (s, 9H) |
| IT195 | 492.2 | ¹H NMR (400 MHz, DMSO-d6) δ: 9.94 (s, 1H), 9.32 (s, 1H), 7.70-7.76 (m, |
| | | 6H), 7.03-7.41 (m, 7H), 5.73 (q, 1H), 3.02 (s, 3H), 2.10 (s, 3H), 1.52 (d, 3H) |
| IT196 | 513.2 | ¹H NMR (400 MHz, DMSO-d6) δ: 9.29 (s, 1H), 7.62-7.74 (m, 4H), |
| | | 7.03-7.40 (m, 5H), 6.74 (d, 2H), 5.73 (q, 1H), 2.68 (m, 2H), 2.38 (m, 2H), 2.09 (s, 3H), |
| | | 1.95 (m, 2H), 1.52 (d, 3H) |
| IT197 | 499.2 | ¹H NMR (400 MHz, DMSO-d6) δ: 9.28 (s, 1H), 7.64-7.75 (m, 4H), |
| | | 7.01-7.40 (m, 5H), 6.99 (d, 2H), 5.73 (q, 1H), 2.10 (s, 3H), 1.52 (m, 5H), 1.26 (m, 2H) |
| IT198 | 534.2 | ¹H NMR (400 MHz, DMSO-d6) δ: 9.58 (s, 1H), 6.94-7.84 (m, 18H), 5.76 (q, |
| | | 1H), 3.60 (s, 3H), 1.51 (m, 3H) |
| IT199 | 536.2 | ¹H NMR (400 MHz, DMSO-d6) δ: 9.32 (s, 1H), 8.21-8.27 (m, 2H), |
| | | 7.74-7.79 (m, 6H), 7.06-7.41 (m, 8H), 5.74 (q, 1H), 2.11 (s, 3H), 1.53 (d, 3H) |
| IT226 | 501.2 | ¹H NMR (400 MHz, DMSO-d6) δ: 9.31 (s, 1H), 7.62-7.76 (m, 6H), |
| | | 7.05-7.40 (m, 5H), 6.90 (d, 2H), 5.73 (q, 1H), 2.09 (s, 3H), 1.53 (m, 9H) |
| IT227 | 536.2 | ¹H NMR (400 MHz, DMSO-d6) δ: 9.34 (s, 1H), 8.92 (s, 1H), 8.57 (d, 1H), |
| | | 7.83 (m, 6H), 7.06-7.41 (m, 7H), 6.89 (d, 1H), 5.74 (q, 1H), 2.10 (s, 3H), 1.53 (d, |
| | | 3H) |
| IT228 | 509.2 | ¹H NMR (400 MHz, DMSO-d6) δ: 9.31 (s, 1H), 8.45 (s, 1H), 7.75 (m, 5H), |
| | | 7.61 (m, 2H), 7.19-7.41 (m, 7H), 7.08 (m, 3H), 5.74 (q, 1H), 2.10 (s, 3H), 1.53 (d, |
| | | 3H) |
| IT229 | 536.2 | ¹H NMR (400 MHz, DMSO-d6) δ: 9.31 (s, 1H), 8.45 (d, 1H), 7.75 (m, 5H), |
| | | 7.61 (m, 2H), 7.19-7.41 (m, 7H), 7.08 (m, 3H), 5.74 (q, 1H), 2.10 (s, 3H), 1.53 (d, |
| | | 3H) |
| IT230 | 537.2 | ¹H NMR (400 MHz, DMSO-d6) δ: 9.44 (s, 1H), 9.16 (s, 1H), 8.93 (s, 1H), |
| | | 7.63-7.90 (m, 6H), 7.06-7.50 (m, 7H), 5.83 (q, 1H), 2.18 (s, 3H), 1.62 (d, 3H) |
| IT231 | 537.2 | ¹H NMR (400 MHz, DMSO-d6) δ: 9.34 (s, 1H), 9.18 (d, 1H), 8.04 (d, 1H), |
| | | 7.79-7.86 (m, 6H), 7.06-7.41 (m, 7H), 5.74 (q, 1H), 2.11 (s, 3H), 1.53 (d, 3H) |
| IT232 | 537.2 | ¹H NMR (400 MHz, DMSO-d6) δ: 9.34 (s, 1H), 8.45 (d, 1H), 8.35 (d, 1H), |
| | | 7.71-7.81 (m, 6H), 7.06-7.41 (m, 7H), 5.74 (q, 1H), 2.11 (s, 3H), 1.53 (d, 3H) |
| IT233 | 481.2 | |
| IT234 | 519.2 | ¹H NMR (400 MHz, DMSO-d6) δ: 9.37 (s, 1H), 7.80-7.88 (m, 4H), 7.49 (d, |
| | | 2H), 7.03-7.41 (m, 5H), 5.73 (q, 1H), 2.10 (s, 3H), 1.52-1.57 (m, 5H), 1.26 (m, |
| | | 1H), 0.81 (m, 1H) |
| IT257 | 536.2 | ¹H NMR (400 MHz, DMSO-d6) δ: 9.27 (s, 1H), 8.55 (d, 1H), 8.48 (s, 1H), |
| | | 7.70-7.58 (m, 7H), 7.18-7.39 (m, 5H), 7.01 (d, 2H), 5.72 (q, 1H), 2.09 (s, 3H), |
| | | 1.51 (d, 3H) |
| IT258 | 572.2 | ¹H NMR (400 MHz, DMSO-d6) δ: 9.34 (s, 1H), 8.26-8.34 (m, 2H), |
| | | 7.70-7.92 (m, 6H), 7.03-7.39 (m, 6H), 5.73 (q, 1H), 2.10 (s, 3H), 1.52 (d, 3H) |
| IT259 | 537.2 | ¹H NMR (400 MHz, DMSO-d6) δ: 9.33 (s, 1H), 8.34 (d, 1H), 8.16 (d, 2H), |
| | | 7.84-7.97 (m, 3H), 7.68 (t, 1H), 7.58 (d, 1H), 7.03-7.43 (m, 5H), 5.72 (q, 1H), |
| | | 2.10 (s, 3H), 1.51 (d, 3H) |
| IT300 | 572.2 | ¹H NMR (400 MHz, DMSO-d6) δ: 9.36 (s, 1H), 8.97 (s, 1H), 8.61 (d, 1H), |
| | | 7.83-7.95 (m, 6H), 7.04-7.40 (m, 6H), 5.73 (q, 1H), 2.10 (s, 3H), 1.52 (d, 3H) |
| IT301 | 482.2 | ¹H NMR (400 MHz, DMSO-d6) δ: 9.84 (s, 1H), 7.84 (d, 1H), 7.61 (m, 2H), |
| | | 7.22-7.35 (m, 8H), 7.08 (d, 1H), 6.81 (d, 2H), 5.75 (q, 1H), 3.58 (s, 3H), 1.47 (d, |
| | | 3H) |
| IT302 | 535.2 | ¹H NMR (400 MHz, DMSO-d6) δ: 9.36 (s, 1H), 7.80-7.88 (m, 4H), 7.65 (d, |
| | | 2H), 7.02-7.41 (m, 5H), 5.74 (q, 1H), 5.17 (t, 1H), 4.45 (m, 1H), 4.25 (m, 1H), |
| | | 2.70 (m, 1H), 2.35 (m, 1H), 2.10 (s, 3H), 1.53 (d, 3H) |
| IT303 | 519.2 | ¹H NMR (400 MHz, DMSO-d6) δ: 9.83 (s, 1H), 8.25-8.33 (m, 2H), 7.66 (s, |
| | | 1H), 7.22-7.37 (m, 8H), 5.78 (q, 1H), 3.62 (s, 3H), 1.51 (d, 3H) |
| IT304 | 535.2 | ¹H NMR (400 MHz, DMSO-d6) δ: 9.35 (s, 1H), 7.77-7.85 (m, 4H), 7.55 (d, |
| | | 2H), 7.04-7.41 (m, 5H), 5.73 (q, 1H), 2.10 (s, 3H), 1.52 (d, 3H), 1.34-1.44 (m, |
| | | 4H) |
| IT305 | 573.2 | ¹H NMR (400 MHz, DMSO-d6) δ: 9.39 (s, 1H), 8.52 (s, 1H), 8.36 (s, 1H), |
| | | 7.76-7.94 (m, 6H), 7.03-7.41 (m, 5H), 5.74 (q, 1H), 2.11 (s, 3H), 1.53 (d, 3H) |
| IT306 | 572.2 | ¹H NMR (400 MHz, DMSO-d6) δ: 10.10 (s, 1H), 7.82-7.89 (m, 6H), 7.61 (d, |
| | | 2H), 7.09-7.42 (m, 5H), 7.03 (m, 1H), 5.76 (q, 1H), 3.84 (s, 3H), 1.56 (d, 3H) |
| IT307 | 572.2 | ¹H NMR (400 MHz, DMSO-d6) δ: 7.62-7.90 (m, 4H), 7.84 (m, 2H), 7.63 (d, |
| | | 2H), 7.22-7.32 (m, 5H), 7.03 (m, 1H), 5.64 (q, 1H), 2.09 (s, 3H), 1.37 (d, 3H) |
| IT308 | 571.2 | ¹H NMR (400 MHz, DMSO-d6) δ: 9.67 (s, 1H), 8.27-8.34 (m, 2H), 7.84 (s, |
| | | 1H), 7.55-7.74 (m, 5H), 7.01-7.41 (m, 7H), 5.76 (q, 1H), 3.61 (s, 3H), 154 (d, |
| | | 3H) |
| IT309 | 535.2 | ¹H NMR (400 MHz, DMSO-d6) δ: 9.94 (s, 1H), 7.57-7.86 (m, 9H), |
| | | 7.16-7.42 (m, 5H), 7.10 (d, 1H), 6.97 (d, 2H), 5.76 (q, 1H), 3.83 (s, 3H), 1.55 (d, 3H) |
| IT310 | 535.2 | ¹H NMR (400 MHz, DMSO-d6) δ: 9.66 (s, 1H), 7.52-7.87 (m, 9H), |
| | | 7.25-7.34 (m, 5H), 7.13 (d, 1H), 6.99 (d, 2H), 5.67 (q, 1H), 2.14 (s, 3H), 1.45 (d, 3H) |
| IT311 | 482.2 | ¹H NMR (400 MHz, DMSO-d6) δ: 9.76 (s, 1H), 7.61 (s, 1H), 7.26-7.36 (m, |
| | | 5H), 6.92 (d, 2H), 5.76 (q, 1H), 3.59 (s, 3H), 1.51 (d, 3H), 1.15 (m, 2H), 0.91 (m, |
| | | 2H) |
| IT312 | 535.2 | ¹H NMR (400 MHz, DMSO-d6) δ: 9.97 (s, 1H), 7.75-7.81 (m, 4H), 7.51 (d, |
| | | 2H), 6.96-7.42 (m, 5H), 5.77 (q, 1H), 3.83 (s, 3H), 1.55 (d, 3H), 1.35-1.43 (m, |
| | | 4H) |
| IT313 | 535.2 | ¹H NMR (400 MHz, DMSO-d6) δ: 9.67 (s, 1H), 7.86 (d, 2H), 7.55 (m, 4H), |
| | | 6.98-7.31 (m, 5H), 5.66 (q, 1H), 2.14 (s, 3H), 1.33-1.46 (m, 7H) |
| IT314 | 534.2 | ¹H NMR (400 MHz, DMSO-d6) δ: 9.62 (s, 1H), 7.81 (s, 1H), 7.64 (d, 2H), |
| | | 7.46-7.52 (m, 4H), 6.99-7.40 (m, 5H), 5.74 (q, 1H), 3.60 (s, 3H), 1.54 (d, 3H), |
| | | 1.41 (m, 2H), 1.35 (m, 2H) |
| IT315 | 571.2 | ¹H NMR (400 MHz, DMSO-d6) δ: 9.35 (s, 1H), 7.87 (d, 1H), 7.54-7.64 (m, |
| | | 5H), 7.04-7.35 (m, 7H), 6.98 (d, 2H), 5.67 (q, 1H), 2.16 (s, 3H), 1.49 (d, 3H) |
| IT316 | 571.2 | ¹H NMR (400 MHz, DMSO-d6) δ: 9.39 (s, 1H), 7.82-7.91 (m, 2H), 7.67 (d, |
| | | 2H), 7.19--7.41 (m, 6H), 7.08 (m, 1H), 6.94 (t, 1H), 6.58 (d, 1H), 5.74 (q, 1H), |
| | | 2.11 (s, 3H), 1.53 (d, 3H) |
| IT406 | 596.2 | ¹H NMR (400 MHz, DMSO-d6) δ: 10.02 (s, 1H), 7.85 (m, 5H), 7.74 (d, 2H), |
| | | 7.19-7.51 (m, 7H), 6.90 (d, 1H), 5.79 (q, 1H), 3.86 (s, 3H), 1.58 (d, 3H) |
| IT407 | 571.2 | ¹H NMR (400 MHz, DMSO-d6) δ: 10.02 (s, 1H), 7.85 (m, 5H), 7.74 (d, 2H), |
| | | 7.19-7.51 (m, 7H), 6.90 (d, 1H), 5.79 (q, 1H), 3.86 (s, 3H), 1.58 (d, 3H) |
| IT408 | 571.2 | ¹H NMR (400 MHz, DMSO-d6) δ: 9.72 (s, 1H), 7.98 (m, 2H), 7.81-7.85 (m, |
| | | 4H), 7.62 (d, 2H), 7.49 (m, 1H), 7.20-7.33 (m, 5H), 6.91 (d, 1H), 5.68 (q, 1H), |
| | | 2.17 (s, 3H), 1.48 (d, 3H) |
| IT423 | 554.2 | ¹H NMR (400 MHz, DMSO-d6) δ: 9.38 (s, 1H), 8.93 (s, 1H), 8.58 (d, 1H), |
| | | 7.84-7.94 (m, 5H), 7.71 (d, 1H), 7.12-7.49 (m, 6H), 6.89 (d, 1H), 5.76 (q, 1H), |
| | | 2.13 (s, 3H), 1.55 (d, 3H) |
| IT424 | 554.2 | ¹H NMR (400 MHz, DMSO-d6) δ: 9.37 (s, 1H), 8.46 (m, 1H), 8.58 (d, 1H), |
| | | 7.84-7.87 (m, 5H), 7.60 (m, 3H), 7.11-7.45 (m, 6H), 5.76 (q, 1H), 2.13 (s, 3H), |
| | | 1.55 (d, 3H) |
| IT425 | 570.2 | ¹H NMR (400 MHz, DMSO-d6) δ: 9.68 (s, 1H), 7.71-7.87 (m, 6H), 7.59 (m, |
| | | 2H), 7.49 (m, 1H), 7.02-7.43 (m, 6H), 6.90 (d, 1H), 5.77 (q, 1H), 3.63 (s, 3H), |
| | | 1.56 (d, 3H) |
| IT454 | 553.2 | ¹H NMR (400 MHz, DMSO-d6) δ: 9.32 (s, 1H), 7.75 (m, 6H), 7.31-7.49 (m, |
| | | 5H), 7.03-7.19 (m, 4H), 6.87 (d, 1H), 5.73 (q, 1H), 2.11 (s, 3H), 1.52 (d, 3H) |
| IT455 | 553.2 | ¹H NMR (400 MHz, DMSO-d6) δ: 9.95 (s, 1H), 7.67-7.77 (m, 6H), |
| | | 7.31-7.48 (m, 5H), 6.97-7.22 (m, 4H), 6.84 (d, 2H), 5.76 (q, 1H), 3.83 (s, 3H), 1.55 (d, |
| | | 3H) |
| IT456 | 552.2 | ¹H NMR (400 MHz, DMSO-d6) δ: 9.61 (s, 1H), 7.80 (s, 1H), 7.31-7.70 (m, |
| | | 11H), 6.97-7.18 (m, 4H), 6.84 (d, 2H), 5.75 (q, 1H), 3.61 (s, 3H), 1.54 (d, 3H) |
| IT457 | 553.2 | ¹H NMR (400 MHz, DMSO-d6) δ: 9.95 (s, 1H), 7.73-7.79 (m, 4H), 7.54 (m, |
| | | 2H), 7.21-7.47 (m, 5H), 6.96-7.20 (m, 4H), 6.84 (d, 2H), 5.66 (q, 1H), 2.14 (s, |
| | | 3H), 1.45 (d, 3H) |
| IT467 | 553.2 | ¹H NMR (400 MHz, DMSO-d6) δ: 9.32 (s, 1H), 7.69-7.82 (m, 7H), |
| | | 7.19-7.41 (m, 5H), 6.99-7.09 (m, 3H), 6.90 (d, 1H), 5.73 (q, 1H), 2.10 (s, 3H), 1.52 (d, |
| | | 3H) |
| IT468 | 553.2 | ¹H NMR (400 MHz, DMSO-d6) δ: 9.95 (s, 1H), 7.92 (m, 1H), 7.67-7.77 (m, |
| | | 6H), 7.21-7.42 (m, 5H), 7.14 (m, 1H), 7.03 (m, 2H), 6.94 (d, 1H), 5.76 (q, 1H), |
| | | 3.83 (s, 3H), 1.55 (d, 3H) |
| IT469 | 552.2 | ¹H NMR (400 MHz, DMSO-d6) δ: 9.61 (s, 1H), 7.87 (m, 1H), 7.79 (s, 1H), |
| | | 7.45-7.66 (m, 6H), 7.24-7.42 (m, 4H), 6.97-7.17 (m, 4H), 6.90 (d, 1H), 5.75 (q, |
| | | 1H), 3.61 (s, 3H), 1.53 (d, 3H) |
| IT459 | 553.2 | ¹H NMR (400 MHz, DMSO-d6) δ: 9.30 (s, 1H), 7.67-7.76 (m, 6H), 7.57 (m, |
| | | 1H), 7.31-7.45 (m, 5H), 7.02-7.19 (m, 2H), 6.95 (d, 2H), 5.73 (q, 1H), 2.10 (s, |
| | | 3H), 1.52 (d, 3H) |
| IT460 | 553.2 | ¹H NMR (400 MHz, DMSO-d6) δ: 9.94 (s, 1H), 7.75 (m, 2H), 7.59-7.66 (m, |
| | | 5H), 7.17-7.47 (m, 7H), 6.95 (d, 2H), 5.76 (q, 1H), 3.83 (s, 3H), 1.55 (d, 3H) |
| IT461 | 552.2 | ¹H NMR (400 MHz, DMSO-d6) δ: 9.60 (s, 1H), 7.87 (s 1H), 7.31-7.62 (m, |
| | | 12H), 6.98-7.22 (m, 2H), 6.93 (d, 2H), 5.75 (q, 1H), 3.61 (s, 3H), 1.53 (d, 3H) |
| IT475 | 494.2 | ¹H NMR (400 MHz, DMSO-d6) δ: 9.72 (s, 1H), 7.91 (s, 1H), 7.80 (d, 1H), |
| | | 6.98-7.47 (m, 9H), 6.75 (m, 1H), 5.75 (q, 1H), 3.62 (s, 3H), 1.52 (d, 3H) |
| IT476 | 600.2 | ¹H NMR (400 MHz, DMSO-d6) δ: 10.01 (s, 1H), 8.35 (m, 2H), 7.62-7.83 (m, |
| | | 6H), 6.97-7.42 (m, 6H), 5.78 (q, 1H), 4.35 (q, 2H), 3.84 (s, 3H), 1.56 (d, 3H), |
| | | 1.33 (t, 3H) |
| IT477 | 563.2 | ¹H NMR (400 MHz, DMSO-d6) δ: 9.94 (s, 1H), 7.64-7.87 (m, 8H), |
| | | 7.36-7.45 (m, 5H), 7.19 (m, 2H), 6.97 (m, 2H), 5.78 (q, 1H), 4.16 (q, 2H), 3.83 (s, 3H), |
| | | 1.56 (d, 3H), 1.33 (t, 3H) |
| IT478 | 549.2 | ¹H NMR (400 MHz, DMSO-d6) δ: 9.94 (s, 1H), 7.62-7.88 (m, 8H), |
| | | 7.36-7.45 (m, 5H), 7.17 (m, 2H), 6.97 (m, 2H), 5.77 (q, 1H), 3.83 (s, 3H), 3.72 (s, 3H), |
| | | 1.56 (d, 3H) |

### Example 41

### Synthesis of Exemplary Compounds of Formula (III)

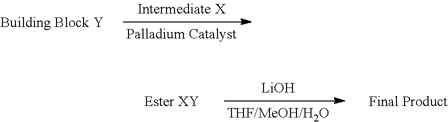

### Synthesis of Intermediate X1

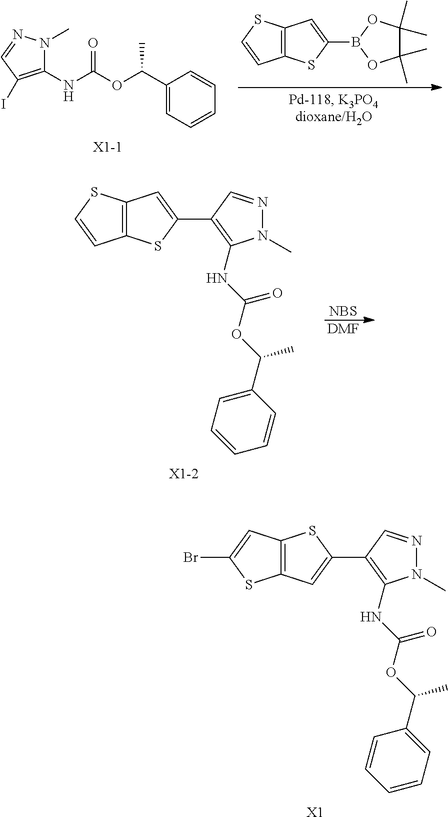

The mixture of 4,4,5,5-tetramethyl-2-(thieno[3,2-b]thiophen-5-yl)-1,3,2-dioxaborolane (6 g, 0.023 mol), XI-1 (8.37 g, 0.023 mol), K₃PO₄ (9.75 g, 0.046 mmol) and Pd-118 (750 mg, 1.15 mmol) in dioxane/H₂O (150 mL, v/v=5/1) was stirred at 80° C. under Ar for 2 h. After concentrated, the residue was partitioned between H₂O and DCM, the aqueous phase was extracted with DCM, and the combined organic layer was washed with brine, dried over Na₂SO₄, concentrated. The residue was purified by chromatography (PE/EA=10/1) on silica gel to provide XI-2 (8.7 g, yield: 98%).

NBS (3.916 g, 0.022 mol) was added to a solution of XI-2 (8.7 g, 0.023 mol) in dry DMF. The mixture was stirred at rt for 1 h. then quenched with H₂O. The product was extracted with CH₂Cl₂. The organic phase was dried with anhydrous sodium sulfate, filtered, and concentrated to give X1 (10.5 g, yield: 99%), which was used to next step without further purification.

### Synthesis of Intermediate X2

Intermediate X2 was prepared following the same procedure as described in the synthesis of X1 using (R)-1-phenylethyl (4-iodo-1-methyl-1H-1,2,3-triazol-5-yl)carbamate in place of XI-1.

### Synthesis of Intermediate X3

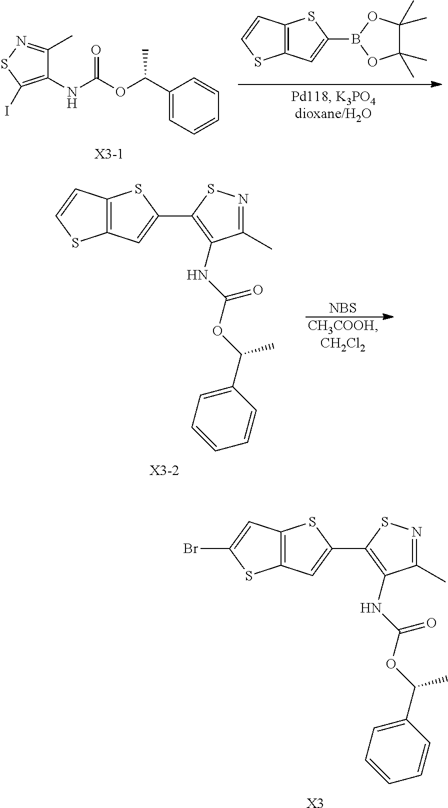

X3-2 was prepared following the same procedure as described in the synthesis of intermediate XI-2 using X3-1 in place of XI-1.

To a solution of X3-2 (82 mg, 0.205 mmol) in CH₃COOH (1 mL) and CH₂Cl₂ (2 mL) was added NBS (36 mg, 0.205 mmol). The mixture was stirred at 10° C. for 2 h. Then the mixture was washed with H₂O. The organics were collected, dried with Na₂SO₄, filtered, and concentrated give crude X3 (98 mg, yield: 99.6%). MS (ESI) m/z (M+2)⁺ 480.5.

### Synthesis of Building Block Y01A (Method A)

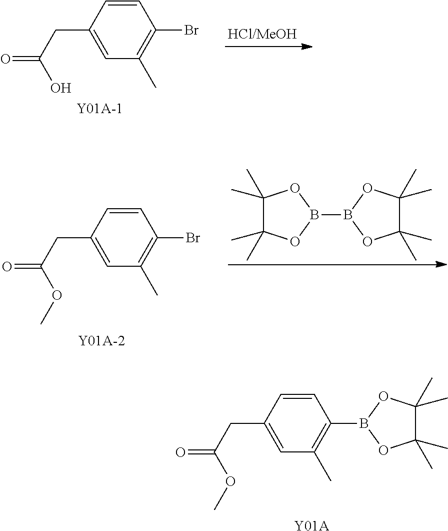

Y01A-1 (5.0 g, 22 mmol) in HCl/MeOH (70 mL) was heated to reflux for 3 h. Then concentrated under reduced pressure to give Y01A-2 (4.8 g, yield: 89.8%). The residue was used directly without further purification.

The mixture of Y01A-2 (4.8 g, 20 mmol), bis(pinacolato)diboron (5.56 g, 22 mmol), AcOK (4.9 g, 50 mmol) and Pd(dppf)Cl₂ (731 mg, 1.0 mmol) in dioxane (100 mL) was heated to reflux under nitrogen for 2 hrs. After concentrated, the residue was partitioned between H₂O and EA, the aqueous phase was extracted with EA (50 mL×3), and the combined organic layer was washed with brine, dried over MgSO₄, concentrated. The residue was purified by column chromatography on silica gel (PE:EA=10:1) to provide Y01A (3.30 g, yield: 57.0%). ¹H NMR (CDCl₃, 400 MHz): δ : 7.71 (d, J=4.0, 1H), 7.06 (d, J=4.0 2H), 3.66 (s, 3H), 3.57 (s, 2H), 2.51 (s, 2H), 1.31 (s, 12H).

### Synthesis of Building Block Y01B (Method B)

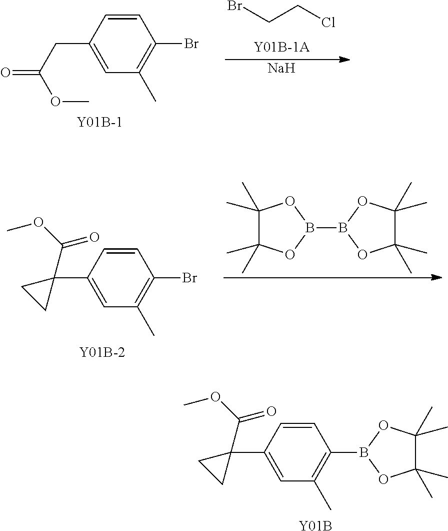

Y01B-1 (4.35 g, 17.9 mmol) and Y01B-1A (3.07 g, 21.5 mmol) in DMSO (50 mL) was added to NaH (2.15 g, 53 mmol) at 0° C. The reaction mixture was stirred for 2 hrs. Water was added and extracted with EtOAc. The combined organic layers were washed with brine and concentrated under reduced pressure, then purified by column chromatography on silica gel (PE:EA=10:1) to provide Y01B-2 (530 mg, yield: 12.7%).

Y01B was prepared by reacting Y01B-2 (530 mg, 2.0 mmol) with bis(pinacolato)diboron (517 mg, 2.2 mmol) following the similar procedure described in the synthesis of Y01A (500 mg, yield: 79.0%). ¹H NMR (CDCl₃, 400 MHz) δ 7.71 (d, J=8.0, 1H), 7.13 (d, J=8.0, 2H), 3.61 (s, 3H), 2.51 (s, 3H), 1.56 (q, J=4.0, 2H), 1.31 (s, 12H), 1.16 (q, J=8.0, 2H).

### Synthesis of Building Block Y02A (Method C)

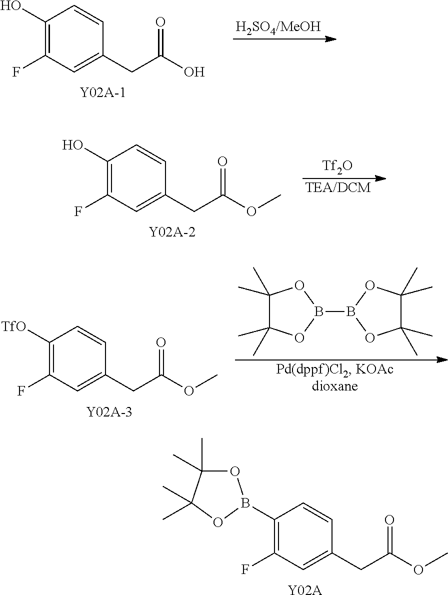

A solution of Y02A-1 (2.0 g, 11.7 mmol) in MeOH (3 mL) was added con.H₂SO₄ (2 drops). The reaction mixture was stirred at 70° C. for 2 h. The reaction mixture was evaporated to yield Y02A-2 (2 g, crude).

To a solution of Y02A-2 (1 g, 5.43 mmol) in DCM (30 mL) was added triethylamine (1.09 g, 10.86 mmol) and (CF₃SO₂)₂O (1.68 g, 5.97 mmol) at −40° C. dropwise. The mixture was stirred at rt for 1 h. The reaction mixture was diluted with water and extracted with EtOAc. The combined organic layers were dried over anhydrous Na₂SO₄ and concentrated under reduced pressure to provide Y02A-3 (1.7 g, crude).

Y02A was prepared by reacting Y02A-3 (1.7 g, 5.34 mmol) with bis(pinacolato)diboron (1.49 g, 5.88 mmol) following the similar procedure described in the synthesis of Y01A (1.2 g, yield: 75.9%).

### Synthesis of Building Block Y02B (Method D)

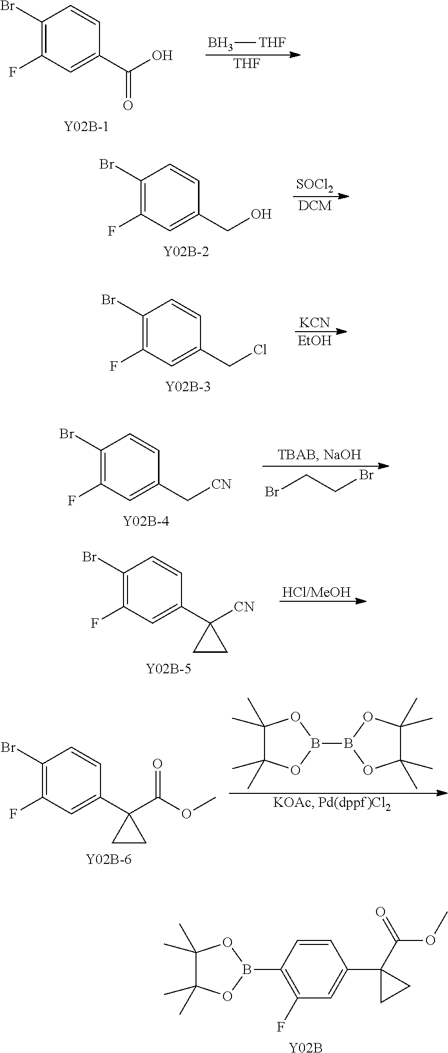

To a stirred solution of Y02B-1 (10 g, 45.87 mmol) in 100 mL of THF was added dropwise BH₃-THF (91.74 mL) at −40° C. The reaction mixture was stirred for 1 h at 50° C. MeOH was added dropwise. The reaction solution was concentrated under reduced pressure to provide Y02B-2 (8 g, yield: 85.56%).

SOCl₂ (9.3 g, 89.8 mmol) was added to a solution of Y02B-2 (8 g, 39.21 mmol) in dry DCM. The mixture was stirred at rt for 2 h then quenched with saturated aqueous NaHCO₃. The product was extracted with CH₂Cl₂. The organic phase was dried with anhydrous Na₂SO₄, filtered, and concentrated to give Y02B-3 (8 g, yield: 91.95%)

The mixture of Y02B-3 (8 g, 36.04 mmol), KCN (6 g, 86.9 mmol) in EtOH (120 mL) was heated to reflux under nitrogen for 4 h. After concentrated, the residue was partitioned between H₂O and EA, the aqueous phase was extracted with EA and the combined organic layer was washed with brine, dried over Na₂SO₄, concentrated. The residue was purified by chromatography (PE/EA=3/1) on silica gel to provide Y02B-4 (4 g, yield: 52.15%).

The mixture of Y02B-4 (2 g, 9.34 mmol) and 1,2-dibromoehane (1.2 mL) in toluene (8 mL), 50% NaOH (8 mL), and TBAB (0.64 g. 0.2 mmol) were added. The mixture was heated to reflux under nitrogen overnight. After concentrated, the residue was partitioned between H₂O and DCM, the aqueous phase was extracted with DCM. The organic layer was washed with 1M HCl and the combined organic layer was washed with brine, dried over Na₂SO₄, concentrated. The residue was purified by chromatography (PE/EA=3/1) on silica gel to provide Y02B-5 (1.3 g, yield: 58.03%).

A solution of Y02B-5 (1.3 g, 5.44 mmol) in MeOH (10 mL) was added HCl/MeOH (20 mL). The reaction mixture was stirred at rt for 30 min. The reaction mixture was filtered, dried in vacuum to give or provide Y02B-6 (600 mg, yield: 42.86%).

Y02B was prepared by reacting Y02B-6 (600 mg, 2.5 mmol) with bis(pinacolato)diboron (765 mg, 3.0 mmol) following the similar procedure described in the synthesis of Y01A (390 mg, crude).

### Synthesis of Building Block Y04B (Method E)

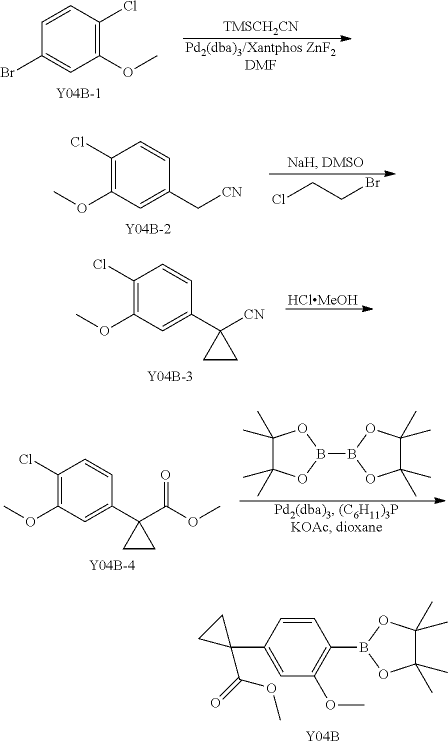

To a solution of Y04B-1 (400 mg, 1.8 mmol) in DMF (5 mL) was added TMSCH₂CN (244 mg, 2.16 mmol), Pd₂(dba)₃ (82.4 mg, 0.09 mmol), Xantphos (52.1 mg, 0.09 mmol) and ZnF₂ (90.9 mg, 0.9 mmol). The mixture was stirred at 90° C. overnight. After concentrated, the mixture was diluted with EtOAc, washed with brine, dried over Na₂SO₄, filtered and concentrated. The residue was purified to give Y04B-2 (180 mg, yield 55%).

To a stirred solution of Y04B-2 (500 mg, 2.76 mmol) in DMSO (10 mL) was added NaH (199 mg, 8.29 mmol). After 1 h, ClCH₂CH₂Br (789 mg, 5.52 mmol) was added to the mixture and stirred for another 1 h. Then the mixture was washed with NH₄Cl and extracted with EtOAc. The organic layer was separated, dried and concentrated. The residue was purified to provide Y04B-3 (300 mg, yield 52.4%).

A mixture of Y04B-3 (300 mg. 1.45 mmol) in HCl/MeOH (20 mL) was stirred at 80° C. for 24 h. After concentrated, the mixture was diluted with EtOAc, washed with brine, dried over Na₂SO₄, filtered and concentrated to yield Y04B-4 (320 mg, yield 92%).

Y04B was prepared by reacting Y04B-4 (245 mg, 1.02 mmol) with bis(pinacolato)diboron (518 mg, 2.04 mmol) following the similar procedure described in the synthesis of Y01A (260 mg, yield 76.7%). ¹H NMR (400 MHz, CDCl₃) δ 7.62 (d, J=7.2 Hz, 1H), 6.93 (d, J=6.8 Hz, 1H), 6.85 (s, 1H), 3.84 (s, 3H), 3.62 (s, 3H), 1.59-1.60 (m, 2H), 1.34 (s, 12H), 1.18-1.21 (m, 2H).

### Synthesis of Building Block Y08A (Method F)

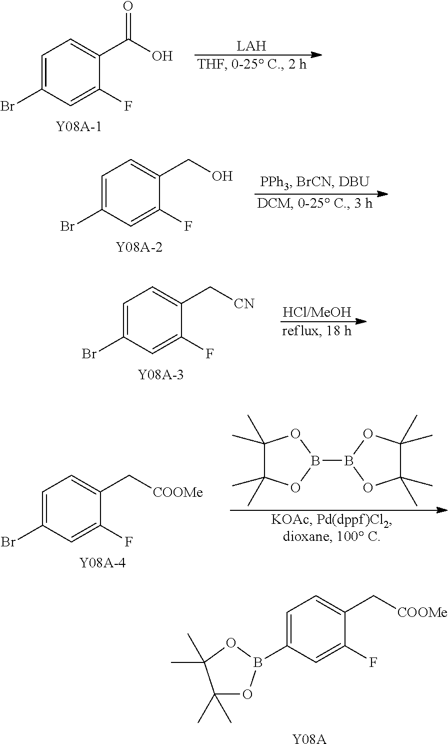

Y08A-3 was prepared following the similar procedure described in the synthesis of Y02B-4. To a mixture of Y08A-3 (5.00 g, 23.5 mmol) in 100 mL of MeOH/HCl was stirred at reflux for 18 h. The mixture was concentrated and purified to give Y08A-4 (6.20 g crude).

Y08A was prepared by reacting Y08A-4 (4.1 g, 16.7 mmol) with bis(pinacolato)diboron (5.09 g, 20.0 mmol) following the similar procedure described in the synthesis of Y01A (5.0 g crude, quant.). ¹H NMR (400 MHz, CDCl₃) δ 7.53 (d, J=7.6 Hz, 1H), 7.48 (s, J=10.0 Hz, 1H), 7.26 (t, J=7.6 Hz, 1H), 3.72 (s, 3H), 3.70 (s, 2H), 1.34 (s, 12H).

### Synthesis of Building Block Y26A (Method G)

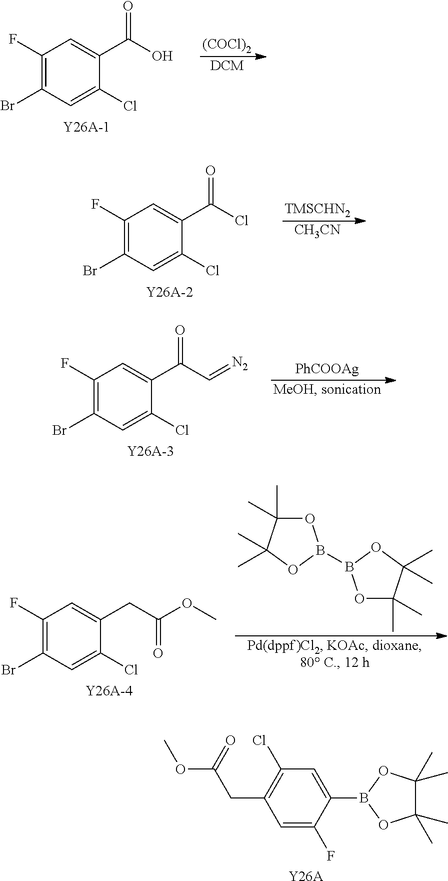

To a solution of Y26A-1 (5 g, 19.84 mmol) in DCM (50 mL) was added DMF (6 drops) and dropwise (COCl)₂ (3.02 g, 23.81 mmol) under nitrogen. The reaction mixture was stirred at rt for 2 h. The mixture was concentrated to give Y26A-2 (5 g, yield: crude).

To a solution of Y26A-2 (5 g, 18.5 mmol) in CH₃CN (50 mL) was added dropwise TMSCHN₂ (11.15 mg, 22.2 mmol) at 0° C. under nitrogen. The reaction mixture was stirred at rt for 18 h and concentrated. The residue was purified to give Y26A-3 (1.5 g, yield: 29.4%).

To a solution of Y26A-3 (1.5 g, 5.43 mmol) in MeOH (15 mL) was added dropwise a solution of PhCOOAg (11.15 mg, 22.2 mmol) in Et₃N (3.03 mL, 21.72 mmol) under nitrogen. The reaction mixture was heated to 50° C. under supersonic for 18 h. The reaction mixture was filtered and concentrated. The residue was purified to give Y26A-4 (860 mg, yield: 56.6%).

Y26A was prepared by reacting Y26A-4 (200 mg, 0.714 mmol) with bis(pinacolato)diboron (199.5 mg, 0.785 mmol) following the similar procedure described in the synthesis of Y01A (90 mg, crude).

### Synthesis of Building Block Y12B (Method H)

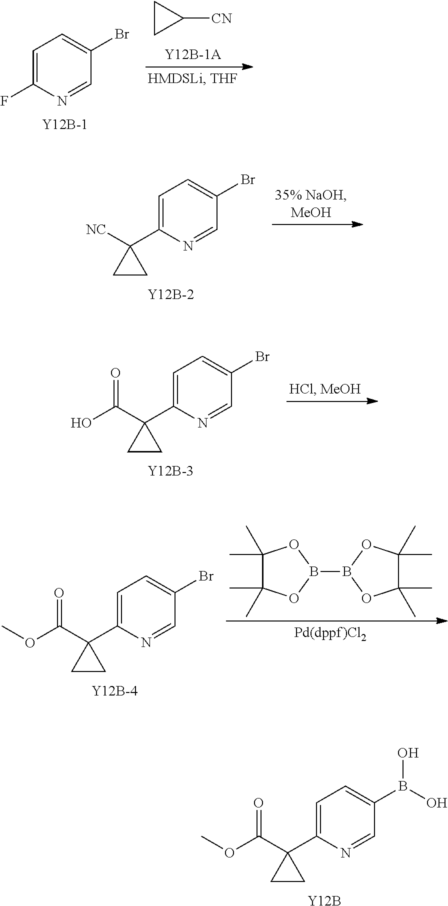

Y12B-1 (10 g, 56.8 mmol) was added to toluene (300 mL). The mixture was then stirred and added to Y12B-1A (12.5 mL, 170 mmol) under nitrogen at 0° C. LiHDMS (176 mL, 176 mmol) was then added drop wise at −5° C. The mixture was warmed to rt and stirred overnight. The mixture was poured into water and filtered. The filtrate was diluted with EA/H₂O, extracted with EA. The combined organic layer was washed with brine, dried and evaporated. The residue was purified to provide Y12B-2 (3 g, yield: 23.8%).

To a stirred solution of Y12B-2 (3 g, 13.5 mmol) in MeOH (25 mL) was added 35% NaOH (50 mL). Then the mixture was heated to 80° C. for 5 h. MeOH was removed in vacuo. The residue was adjusted to pH=7 with HCl and extracted with EtOAc. The combined organic layer was washed with brine, dried and evaporated to provide Y12B-3 (3.4 g, crude, yield: 95%).

To a stirred solution of Y12B-3 (3.4 g, crude) in MeOH (50 mL) was added HCl (5 mL). Then the mixture was heated to reflux for 8 h. After concentrated, the residue was purified to provide Y12B-4 (1.2 g, yield: 34%).

Y12B was prepared by reacting Y12B-4 (1 g, 3.9 mmol) with bis(pinacolato)diboron (1.2 g, 4.7 mmol) following the similar procedure described in the synthesis of Y01A (1.2 g, crude). MS (ESI) m/z (M+H)⁺ 222.0.

### Synthesis of Building Block Y14B (Method I)

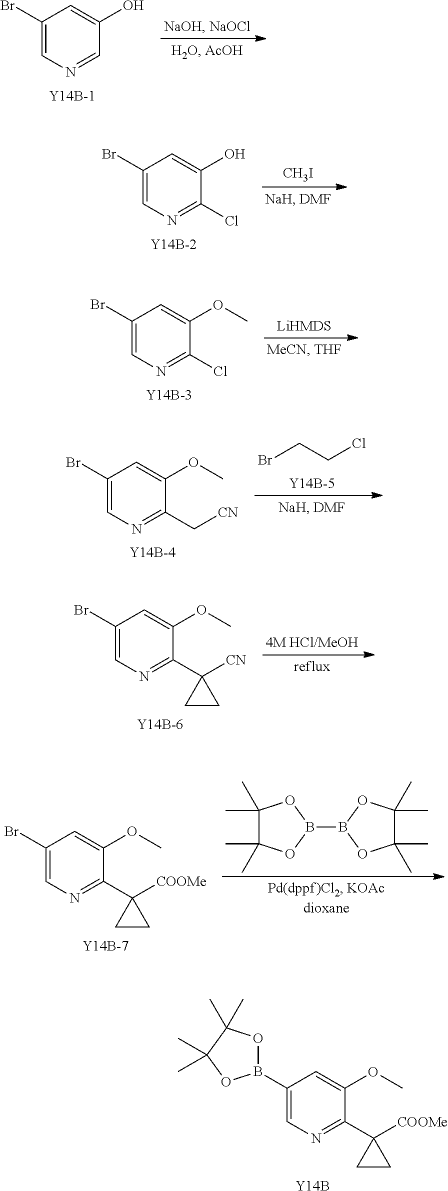

To a solution of Y14B-1 (50 g, 0.289 mol) in 1N NaOH (12.7 g, 0.318 mol), then a 10% solution of NaOCl (175 mL) was added. The mixture was stirred at rt overnight. AcOH was added to bring the mixture pH=7. Then filtered, the filtrate cake was dried in vacuo to give Y14B-2 (50 g, 84% yield).

To a solution of Y14B-2 (50 g, 0.24 mol) in 200 mL DMF was added NaH (11.5 g, 0.29 mol). The mixture was stirred at rt for 30 mins. Then CH₃I (41 g, 0.29 mol) was added. The mixture was stirred at rt for 1 h. After completion of the reaction indicated by TLC, the mixture was poured into brine and extracted with EA. The organic layer was washed with brine, dried over Na₂SO₄, concentrated in vacuo. The crude residue was purified to provide Y14B-3 (35 g, 66% yield).

To a solution of Y14B-3 (18 g, 81.4 mmol) and MeCN (4.2 mL, 81.4 mmol) in 100 mL THF was added 1M LiHMDS (162 mL, 162 mmol). The mixture was stirred at rt for 1 h under N₂. Then MeCN (4.2 mL, 81.4 mmol) followed by 1M LiHMDS (162 mL, 162 mmol) was added. The mixture was stirred at rt for 2 h. The mixture was poured into ice-water and extracted with EA. The organic layer was dried over Na₂SO₄, concentrated in vacuo. The crude residue was purified to provide Y14B-4 (8 g, 44% yield).

To a solution of Y14B-4 (4 g, 17.7 mmol) in50 mL DMF was added NaH (1.4 g, 35.4 mmol). The mixture was stirred at rt for 30 mins. Then Y14B-5 (2.5 g, 17.7 mmol) was added. The mixture was stirred at rt overnight. The mixture was poured into brine, extracted with EA. The organic layer was washed with brine, dried over Na₂SO₄, concentrated in vacuo. The crude residue was purified to provide Y14B-6 (4 g, 90% yield).

To a solution of Y14B-6 (4 g, 15.87 mmol) in 50 mL of 4N HCl/MeOH, the mixture was heated to reflux overnight. The mixture was concentrated under reduced pressure. NaHCO₃ (aq.) was added to bring the mixture pH=8. After extracted with EA, the organic layer was dried over Na₂SO₄, concentrated in vacuo. The crude residue was purified to provide Y14B-7 (4 g, 89% yield).

Y14B was prepared by reacting Y14B-7 (4.0 g, 14 mmol) with bis(pinacolato)diboron (5.1 g, 21 mmol) following the similar procedure described in the synthesis of Y01A (3 g, yield 65%). ¹H NMR (CDCl₃, 300 MHz): δ 8.45 (s, 1H), 7.48 (s, 1H), 3.87 (s, 3H), 3.60 (s, 3H), 1.62 (q, 2H), 1.36 (q, 2H), 1.34 (s, 12H).

### Synthesis of Building Block Y16A (Method J)

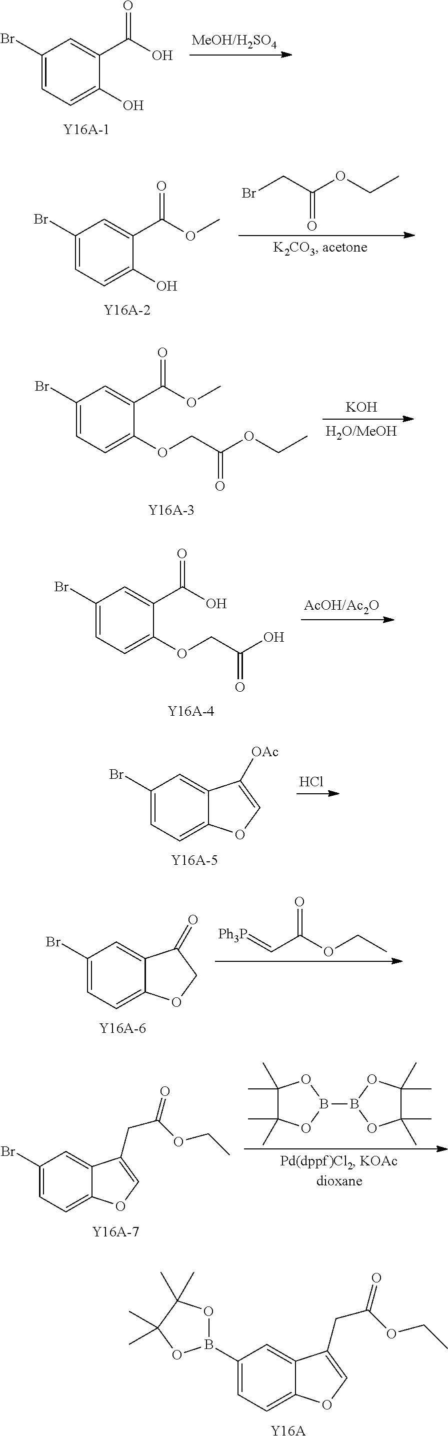

To a solution of Y16A-1 (100 g, 462.9 mmol) in MeOH (500 mL) was added concentrated sulfuric acid (40 mL) at rt. After addition, the solution was refluxed for 12 h. The mixture was poured into crashed ice, basified to pH=8 with solid sodium bicarbonate and extracted with EtOAc. The combined organic layers were dried over sodium sulfate and filtered. The filtrate was concentrated to give Y16A-2 (106 g, yield: 99.5%) as a white solid.

A mixture of Y16A-2 (106 g, 460.9 mmol), ethyl 2-bromoacetate (93.5 g, 563.4 mmol) and potassium carbonate (194.4 g, 1408.8 mmol) in acetone (1 L) was refluxed for 3 h. After cooling to rt, the mixture was partitioned between water and EtOAc. The organic layer was separated and the aqueous layer was extracted with EtOAc. The combined organic layers were dried over sodium sulfate and filtered. The filtrate was concentrated to give crude Y16A-3 (162 g, crude) as a yellowish oil, which solidified upon cooling to rt.

A mixture of Y16A-3 (162 g, crude) in MeOH (1 L) was added aqueous KOH solution (8.53 mmol/mL, 300 mL) dropwise at rt. After addition, the mixture was stirred under reflux for 12 h. MeOH was removed under reduced pressure. The aqueous layer was extracted with EtOAc and the organic layer was discarded. The aqueous layer was poured into crashed ice and acidified to pH=2 with concentrated HCl. The resulting precipitate was filtered and the filter cake was dried to give Y16A-4 (120 g, yield: 95% over two steps) as a white solid.

To a suspension of Y16A-4 (120 g, 438 mmol) in a mixed solvents of AcOH (500 mL) and Ac₂O (500 mL) was added sodium acetate (310 g, 3.78 mol) at rt. The mixture was refluxed for 24 h. The mixture was concentrated. The residue was dissolved in water and the mixture was extracted with DCM. The combined organic layers were dried over sodium sulfate and concentrated to provide a brown liquid Y16A-5, which was used for the next step without further purification.

To a solution of crude Y16A-5 obtained above in MeOH (800 mL) was added 1 M HCl (800 mL) at rt. After addition, the mixture was refluxed for 4 h. MeOH was removed under reduced pressure. The resulting solid was purified to give Y16A-6 (25 g, yield: 26.9% over two steps) as a reddish solid.

To a solution of Y16A-6 (9 g, 42.5 mmol) in toluene (200 mL) was added (ethoxycarbonylmethylene)triphenylphosphorane (18 g, 51.7 mmol) at rt. The mixture was purged with N₂ three times and then refluxed for 16 h. The mixture was concentrated and the residue was purified to give Y16A-7 (6 g, yield: 42.9%) as a yellowish oil.

Y16A was prepared by reacting Y16A-7 (3 g, 10.6 mmol) with bis(pinacolato)diboron (3.2 g, 12.8 mmol) following the similar procedure described in the synthesis of Y01A (1.8 g, yield: 51.4%) as a yellowish oil. ¹H NMR (CDCl₃, 400 MHz) δ 8.06 (s, 1H), 7.78 (d, 1H, J=8.4 Hz), 7.64 (s, 1H), 7.48 (d, 1H, J=8.4 Hz), 4.17-4.22 (q, 2H), 3.72 (s, 2H), 1.36 (s, 12H), 1.27-1.30 (t, 3H, J=7.2 Hz). MS (ESI) m/z [M+H]⁺331.2.

### Synthesis of Building Block Y18A (Method K)

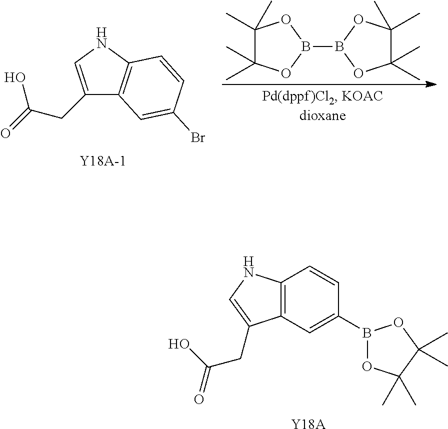

Y18A was prepared by reacting Y18A-1 (100 mg, 0.394 mmol) with bis(pinacolato)diboron (100 mg, 0.394 mmol) following the similar procedure described in the synthesis of Y01A.

### Synthesis of Building Block Y25A (Method L)

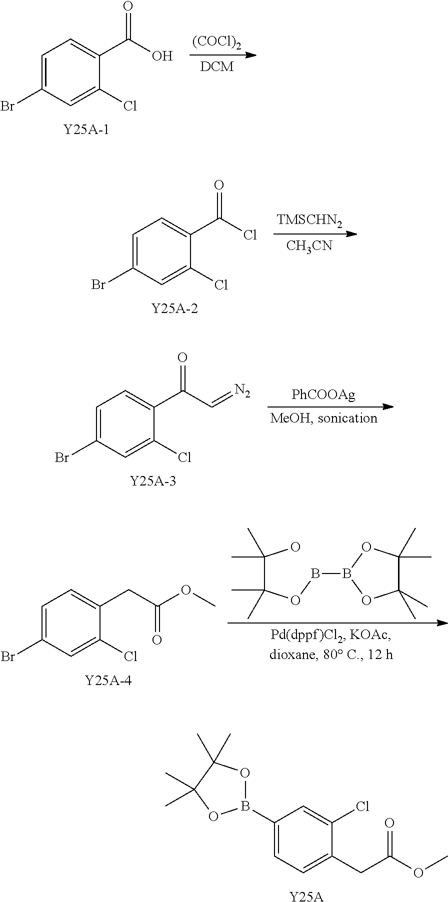

Y25A was prepared following the similar procedure described in the synthesis of Y26A (260 mg, yield 44.8%).

### Synthesis of Building Block Y27A (Method M)

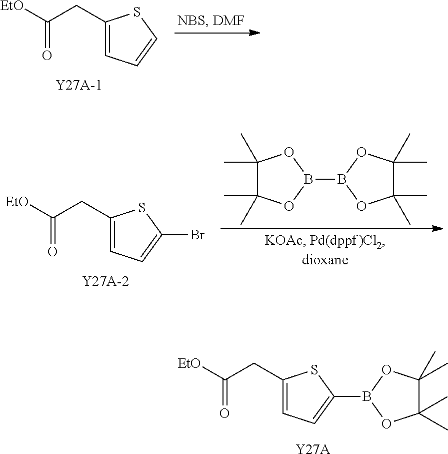

To a stirred solution of Y27A-1 (5 g, 32 mmol) in DMF (60 mL) was added NBS (5.13 g, 29 mmol). Then the solution was heated to 60° C. for 3 h. After being cooled to rt, the mixture was diluted with water and extracted with EtOAc. The combined organic layers were washed with brine, and concentrated under reduced pressure. The residue was purified to give Y27A-2 (6 g, yield: 82%).

Y27A was prepared by reacting Y27A-2 (500 mg, 2.2 mmol) with bis(pinacolato)diboron (637 mg, 2.5 mmol) following the similar procedure described in the synthesis of Y01A (200 mg, yield: 32%). ¹H NMR (CDCl₃, 400 MHz): δ 7.51-7.52 (d, J=3.2 Hz, 1H), 7.04-7.05 (d, J=3.2 Hz, 1H), 4.14-4.25 (m, 2H), 3.87 (s, 2H), 1.35 (s, 12H), 1.28-1.31 (m, 3H).

### Synthesis of Building Block Y27B (Method N)

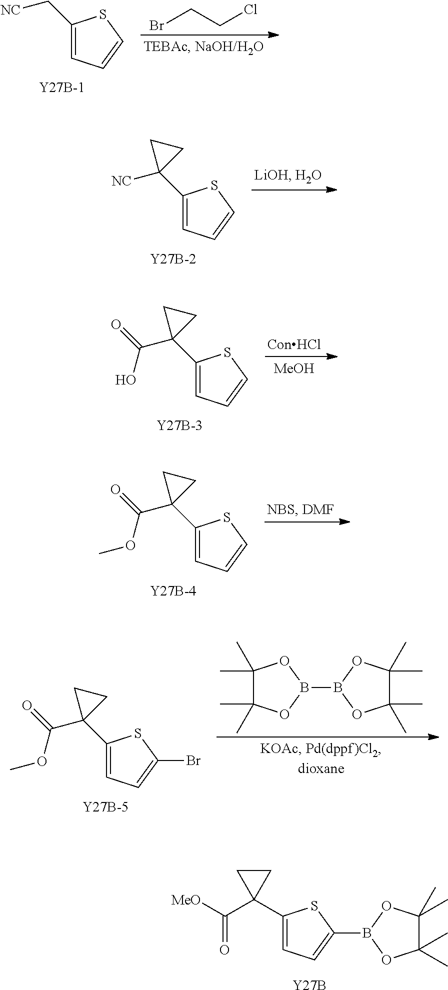

To a mixture of Y27B-1 (5.0 g, 40.65 mmol), 1-bromo-2-chloroethane (8.8 g, 61.97 mmol) and TEBAc (185.0 mg, 0.81 mmol) at 50° C. was added dropwise NaOH (8.1 g, 202.50 mmol) in water (8 mL). Then the mixture was stirred at 50° C. for 3 h. After being cooled to rt, the mixture was diluted with DCM and water. The separated aqueous layer was extracted with DCM. The combined organic layers were washed with brine, dried over Na₂SO₄, and concentrated. The residue was purified to give Y27B-2 (5.6 g, yield: 92%) as a yellow oil.

A mixture of Y27B-2 (3.0 g, 20.13 mmol) in 4.0 N LiOH (160 mL) was stirred at reflux for 4 h. After being cooled to rt, the mixture was adjusted PH to 2 with con. HCl, extracted with DCM. The combined organic layers were washed with brine, dried over Na₂SO₄, and concentrated to give Y27B-3 (3.0 g, yield: 89%) as a brown solid.

To a stirred solution of Y27B-3 (3.0 g, 17.86 mmol) in dry MeOH (40 mL) was added con. HCl (0.2 mL, 1.8 mmol). The mixture was stirred at reflux for 18 h. After evaporation, the residue was diluted with DCM and water. The separated organic layer was washed with sat.aq. NaHCO₃, brine, dried over Na₂SO₄, and concentrated to afford Y27B-4 (2.8 g, yield 86%).

Y27B-5 was prepared by reacting NBS (1.96 g, 10.99 mmol) with Y27B-4 (2.0 g, 10.99 mmol) following the similar procedure described in the synthesis of Y27A-2 (2.4 g, yield: 84%) as a yellow oil.

Y27B was prepared by reacting Y27B-5 with bis(pinacolato)diboron following the similar procedure described in the synthesis of Y01A.

### Synthesis of Building Block Y30B (Method O)

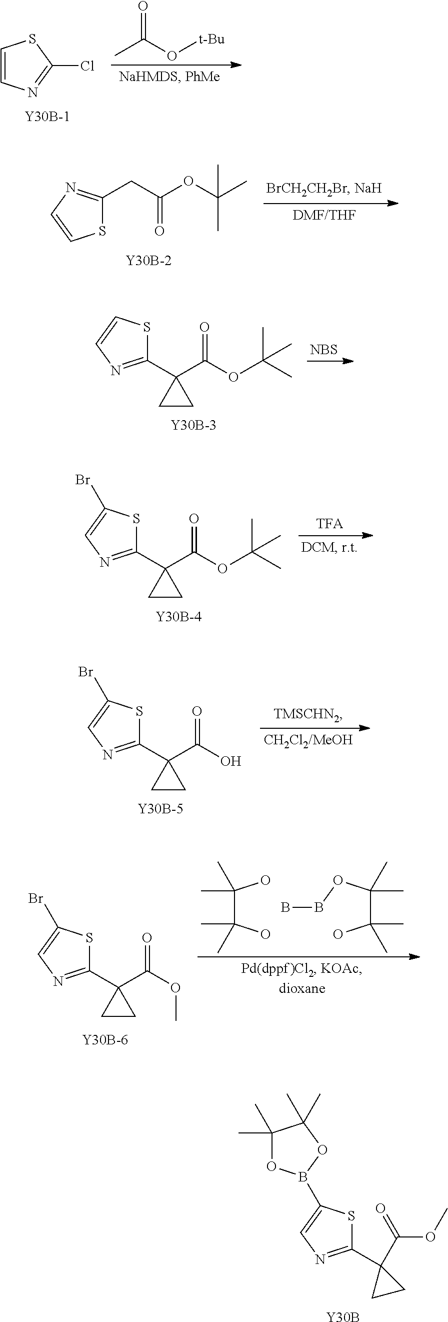

NaHMDS (300 mL, 0.6 M in toluene, 180.6 mmol) was added to the solution of Y30B-1 (9 g, 75.3 mmol) and t-butyl acetate (11.76 g, 90.3 mmol) at 0° C. under N₂. The reaction was stirred for 2 h at 0° C., allowed to warm to 25° C. and stirred for 18 h. Then the reaction was quenched with aqueous saturated NH₄Cl. The mixture was extracted with EA and the combined organic layers were washed with brine, dried and concentrated under vacuum. The residue was purified to obtain Y30B-2 (12 g, yield 65%).

NaH (5.03 g, 209.6 mmol) was suspended in THF (15 mL)/DMF (15 mL) under nitrogen and cooled to 0° C. The solution of Y30B-2 (10.31 g, 51.7 mmol) in THF (5 mL)/DMF (5 mL) was added dropwise. Then the ice-bath was removed and the yellow-orange suspension was stirred at rt for 30 min. The mixture was cooled back down to 0° C., and 1,2-dibromoethane (28.77 g, 153.1 mmol) was added in one portion. The reaction was allowed to warm to rt and stirred for 2 h, then quenched with saturated NH₄Cl aq. solution and extracted with EA. The combined organic layers were washed with brine, dried over Na₂SO₄ and evaporated. The residue was purified to give Y30B-3 (10.8 g, yield 93%).

Y30B-4 was prepared by reacting NBS (7.64 g, 42.9 mmol) with Y30B-3 (8.00 g, 35.4 mmol) following the similar procedure described in the synthesis of Y27A-2 (10 g, yield 92%).

To a solution of Y30B-4 (3.0 g, 9.9 mmol) in DCM (20 mL) and TFA (20 mL) was stirred at 10° C. for 16 h. The mixture was washed with H₂O, extracted with EtOAc. The organics were combined, dried with Na₂SO₄, filtered and concentrated to yield Y30B-5 (2.28 g, yield 93.2%) as a brown solid.

To a stirred solution of Y30B-5 (1.28 g, 5.16 mmol) in DCM/MeOH=1/1 (20 mL) was added TMSCHN₂ (3.87 ml, 7.74 mmol) dropwise at 15° C. The mixture was stirred at 15° C. for 2 h and quenched with MeOH. The solvent was removed by vacuo to give Y30B-6 (992 mg, yield 73.5%) as a brown solid.

Y30B was prepared by reacting Y30B-6 (500 mg, 1.91 mmol) with bis(pinacolato)diboron (726 mg, 2.86 mmol) following the similar procedure described in the synthesis of Y01A (260 mg, yield 44.8%).

### Synthesis of Building Block Y31A (Method P)

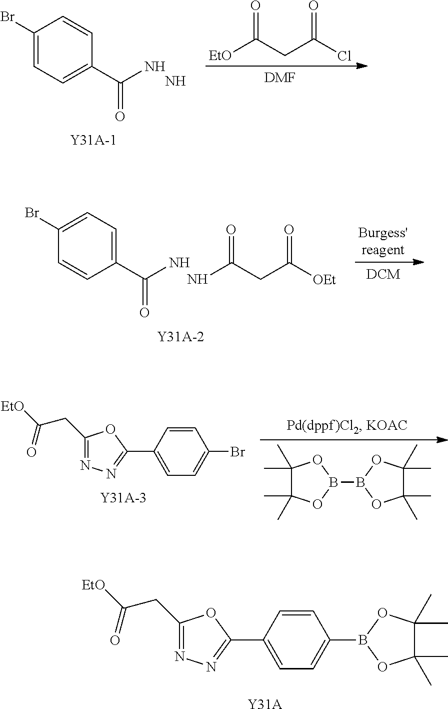

To a stirred solution of Y31A-1 (25 g, 0.12 mol) in DMF (300 mL) was added ethyl 3-chloro-3-oxopropanoate (19.6 g, 0.13 mol) at rt and the mixture was stirred at rt for 3 h. After concentrated, the residue was washed with EtOAc, the organic layer was filtered and the residue was dried to provide Y31A-2 (24 g, yield: 62.7%).

Y31A-2 (500 mg, 1.52 mmol), Burgess' reagent (727 mg, 3.04 mmol) in DCM (5 mL) was heated to 100° C. for 1 h in microwave. The mixture was concentrated and purified to provide Y31A-3 (200 mg, yield: 42%).

Y31A was prepared by reacting Y31A-3 (212 mg, 0.68 mmol) with bis(pinacolato)diboron (208 mg, 0.82 mmol) following the similar procedure described in the synthesis of Y01A (187 mg, yield: 76.6%). MS (ESI) m/z (M+H)⁺ 359.1. ¹H NMR (CDCl₃, 400 MHz): δ 8.05 (d, J=8 Hz, 2H), 7.94 (d, J=8 Hz, 2H), 4.23-4.28 (m, 2H), 4.04 (s, 2H), 1.37 (s, 12H), 1.28-1.31 (t, J=7.2 Hz, 3H).

### Synthesis of Building Block Y32A (Method Q)

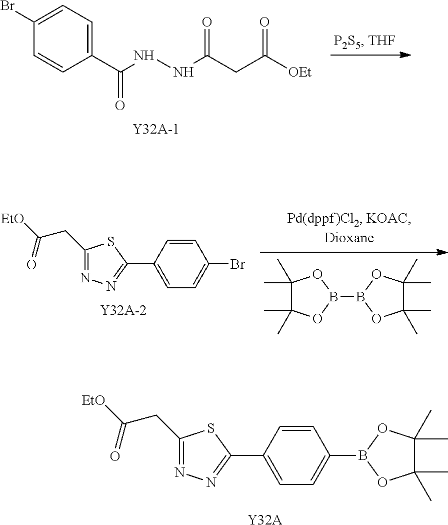

Y32A-1 (2 g, 6.08 mmol), P₂S₅ (2.97 g, 13.38 mmol) in THF (20 mL) was heated to 150° C. for 20 min in microwave. The mixture was concentrated and purified to provide Y32A-2 (975 mg, yield: 48.8%).

Y32A was prepared by reacting Y32A-2 (700 mg, 1.98 mmol) with bis(pinacolato)diboron (605 mg, 2.38 mmol) following the similar procedure described in the synthesis of Y01A.

### Synthesis of Building Block Y33A (Method R)

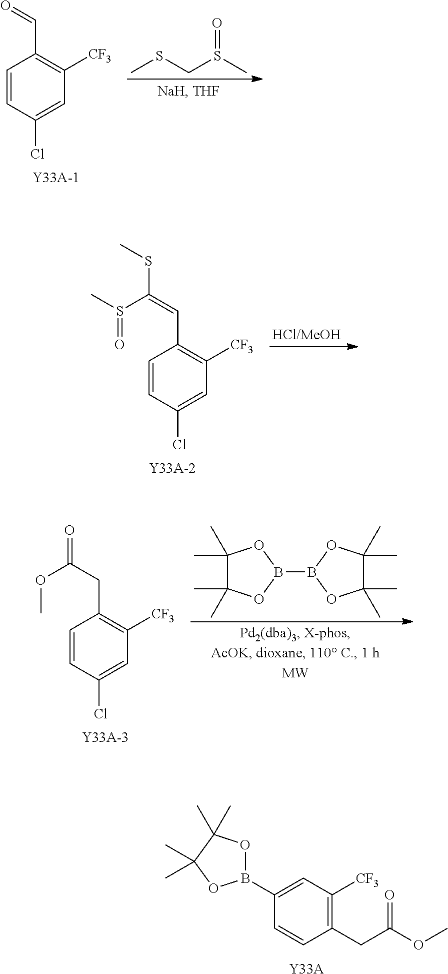

A stirred solution of methyl methylsulfinylmethyl sulfide (1.8 g, 14.4 mmoL) in dry THF (40 mL) was added NaH (0.6 g, 14.4 mmoL, 60% in mineral) at 0° C. The mixture was stirred for 30 min at 0° C. and then Y33A-1 (2.0 g, 9.62 mmol) was added. The reaction mixture was stirred for 1 h at 60° C. Water and EtOAc were added. The organic layer was separated and the aqueous layer was extracted with EtOAc. The organic layers were combined, washed with brine, dried (MgSO₄), filtered and the solvent was evaporated. The residue was purified to give Y33A-2 (900 mg, yield 30%).

A stirred solution of Y33A-2 (0.9 g, 2.87 mmol) in methanol (5 mL) was added HCl (2 mL, 8 mmol, 4M in EtOAc). The mixture was heated to 80° C. for 5 h. The mixture was quenched with saturated aqueous NaHCO₃ (10 mL) and extracted with EtOAc. The organic layers were combined, washed with brine, dried (MgSO₄), filtered and the solvent was evaporated. The residue was purified to give Y33A-3 (0.4 g, yield 55%).

Y33A was prepared by reacting Y33A-3 (200 mg, 0.790 mmol) with bis(pinacolato)diboron (0.442 mg, 1.74 mmol) following the similar procedure described in the synthesis of Y01A (196 mg, yield 72.1%).

### Synthesis of Building Block Y33A (Method S)

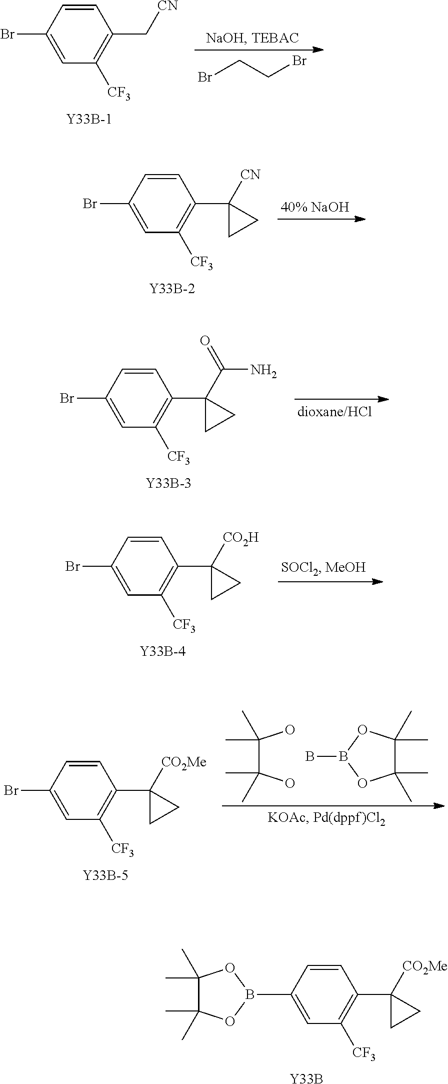

The mixture of Y33B-1 (2 g, 7.6 mmol), 1,2-dibromoethane (2.14 g, 11.4 mmol) and TEBAC (173 mg, 0.76 mmol) in toluene (10 mL) was added 50% NaOH (10 mL) dropwise at rt. The mixture was stirred at 60° C. for 15 h. Then the mixture was partitioned between H₂O and EA, the aqueous phase was extracted with EA. The combined organic layers were washed with brine, dried over Na₂SO₄, concentrated and purified to give Y33B-2 (1.5 g, yield: 68%) as a yellow solid.

A solution of Y33B-2 (1.5 g, 5.2 mmol) in 40% NaOH (20 mL) was heated to 110° C. for 40 hs. TLC showed the reaction was complete, then the precipitated solid was collected by filtration and washed with water and PE to afford Y33B-3 (1.5 g, yield: 94%) as a red solid.

A solution of Y33B-3 (400 mg, 1.3 mmol) in HCl (4 mL) and dioxane (6 mL) was heated to 110° C. for 15 hs. Then H₂O was added and extracted with EA. The combined organic layers were washed with brine, dried over Na₂SO₄, concentrated to afford crude Y33B-4 (400 mg, crude) as a yellow oil.

To a stirred solution of Y33B-4 (1.2 g, 3.88 mmol) in MeOH (15 mL) was added SOCl₂ (0.6 mL) dropwise at 5° C. Then the mixture was heated at 90° C. for 15 hs. After concentrated, H₂O was added and extracted with EA. The combined organic layers were washed with brine, dried over Na₂SO₄, concentrated and purified to afford Y33B-5 (560 mg, yield: 45%) as a yellow oil.

The mixture of Y33B-5 (550 mg, 1.71 mmol), KOAc (335 mg, 3.42 mmol), bis(pinacolato)diboron (521 mg, 2.05 mmol) and Pd(dppf)Cl₂ (249 mg, 0.34 mmol) in dioxane (10 mL) was heated at 80° C. under nitrogen for 4 hs. Then H₂O was added, and extracted with EA. The combined organic layers were washed with brine, dried over Na₂SO₄, concentrated and purified to afford compound Y33B (400 mg, yield: 63.3%) as a yellow oil.

### Synthesis of Building Block Y48A (Method T)

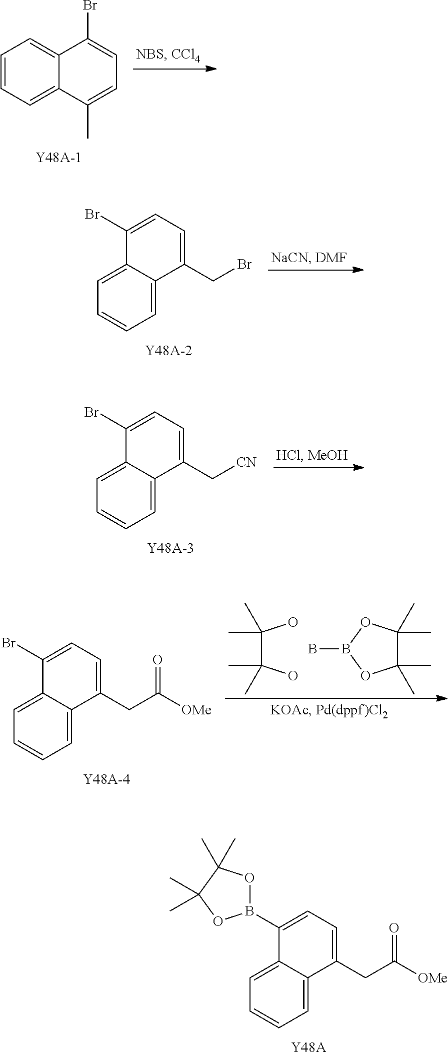

To a stirred solution of Y48A-1 (15 g, 68.2 mmol) in CCl₄ (150 mL) was added BPO (594 mg, 2.5 mmol) and NBS (13.3 g, 75 mmol). Then the reaction mixture was heated to reflux for 15 h under N₂. Then cooled to rt, filtered and the filtrate concentrated under reduced pressure to afford the Y48A-2 (20 g, yield: 97%) as a yellow solid.

To a solution of Y48A-2 (15 g, 0.05 mol) in DMF (90 mL) and H₂O (10 mL) was added NaCN (2.94 g, 0.06 mol). Then the mixture was stirred at 50° C. for 15 h. Then the mixture was partitioned between saturated NaHCO₃ and EA. The aqueous phase was extracted with EA and the combined organic layer was washed with brine, dried over Na₂SO₄, concentrated and purified to afford Y48A-3 (8.9 g, yield: 72%) as a yellow solid.

The preparation of Y48A-4 and Y48A were followed the similar procedure described in Method F.

### Synthesis of Building Block Y46A (Method U)

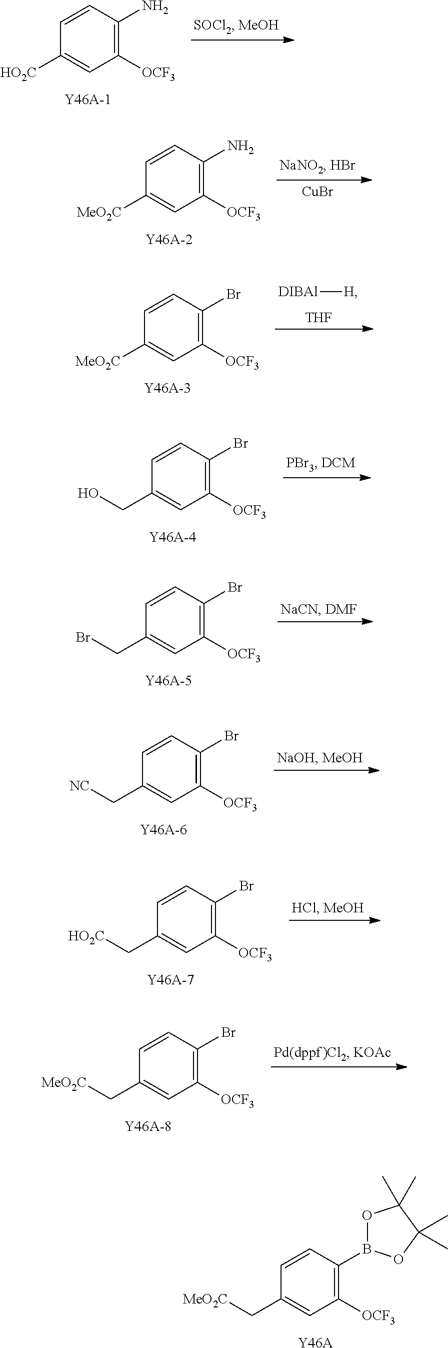

To a stirred solution of Y46-1 (10 g, 42.6 mmol) in SOCl₂ (30 mL) was stirred at reflux for 10 hs. The solvent was removed and the residue was diluted with MeOH at 0° C. The mixture was concentrated and diluted with DCM, washed with NaHCO₃ solution, the organic layer was dried over anhydrous Na₂SO₄, concentrated to provide Y46-2 (12 g, crude, yield: 92.3%) as a brown oil.

To a stirred solution of Y46-2 (12 g, 51.1 mmol) in HBr (100 mL) was added NaNO₂ solution (30 mL, 3M) at 0° C. After 10 min, CuBr (7.3 g, 51.1 mmol) was added. The mixture was stirred at 0° C. for 20 min, H₂O was added, and extracted with EA. The organic layer was dried over anhydrous Na₂SO₄, concentrated and purified to provide Y46-3 (11 g, yield: 72%) as a brown red oil.

To a solution of Y46-3 (3.0 g, 10.1 mmol) in THF (10 mL) was added DIBA1-H (30 mL, 30 mmol) at −60° C. for 30 min. H₂O and NaHCO₃ were added, filtered, washed with DCM. The organic layer was dried over Na₂SO₄, concentrated to provide Y46-4 (2.5 g, yield: 93%) as a colorless oil.

The detailed procedures for preparing the other intermediates and Y46A were similar to those described in Method F.

### Synthesis of Building Block Y36A (Method V)

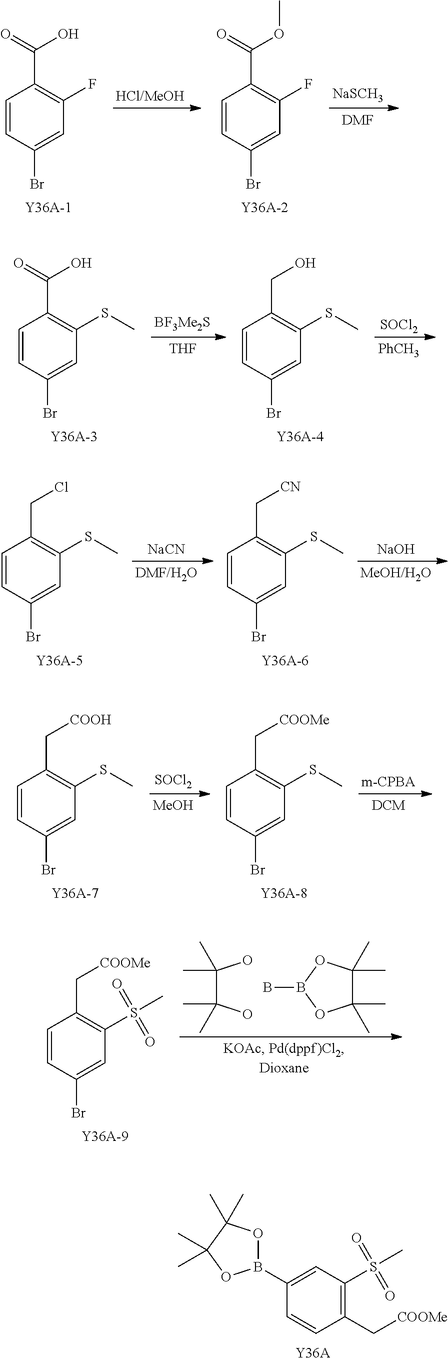

To a stirred solution of Y36A-1 (30.0 g, 0.14 mol) in MeOH (300 mL) was added con. HCl (4 mL). The mixture was stirred at reflux for 20 hs. After evaporation, the residue was diluted with EA and water. The separated organic layer was washed with sat.aq NaHCO₃, brine, dried over Na₂SO₄, and concentrated to afford Y36A-2 (28.0 g, yield 86%).

A mixture of Y36A 2 (30.0 g, 129.34 mmol) and NaSCH₃ (45 g, 646.69 mmol, 20% aq.) in DMF (200 mL) was stirred at 80° C. for 18 h. After cooling to 0° C., the mixture was adjusted to pH=2˜3 with con. HCl, and extracted with EA. The combined organic layer was washed with brine, dried over Na₂SO₄, concentrated to give Y36A-3 (21.0 g, crude).

The procedure for the preparation of Y36A-6 from Y36A-3 was similar to those described in Method D.

To a stirred solution of Y36A-6 (2.0 g, 7.30 mmol) in DCM (30 mL) was added m-CPBA (2.5 g, 14.60 mmol) at 0° C. The mixture was stirred at rt for 18 h. 5% aq.Na₂SO₃ (20 mL) was added to the mixture to quench the excess m-CPBA, and then the mixture was extracted with DCM. The organic layer was washed with brine, dried over Na₂SO₄, concentrated. The residue was purified to give Y36A-7 (500 mg, 22%).

The preparation of Y36A from Y36A-9 and bis(pinacolato)diboron was similar to the procedure described in the synthesis of Y33B in Method S.

### Synthesis of Building Block Y36B (Method W)

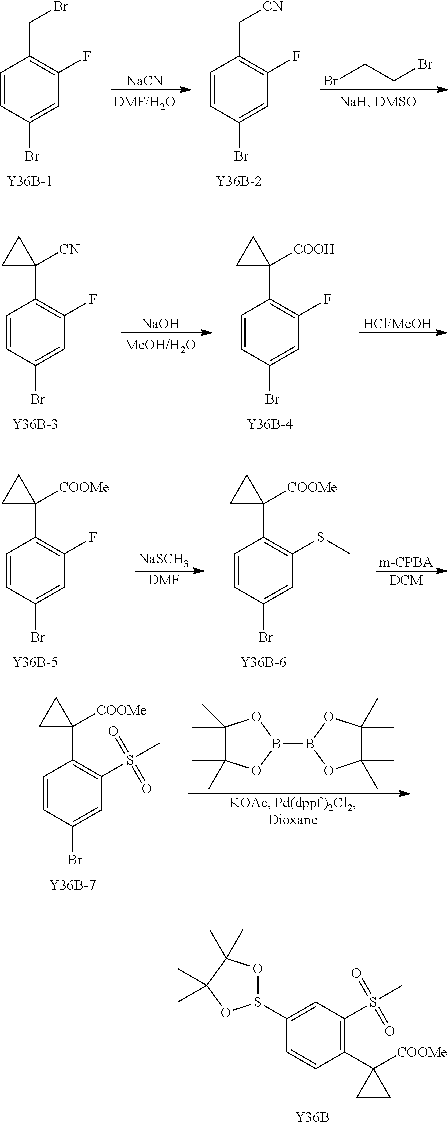

The preparation of Y36B-5 from Y36B-1 was similar to the procedure described in Method D. The preparation of Y36B-7 from Y36B-5 was similar to the procedure described in in Method V.

### Synthesis of Building Block Y45A (Method X)

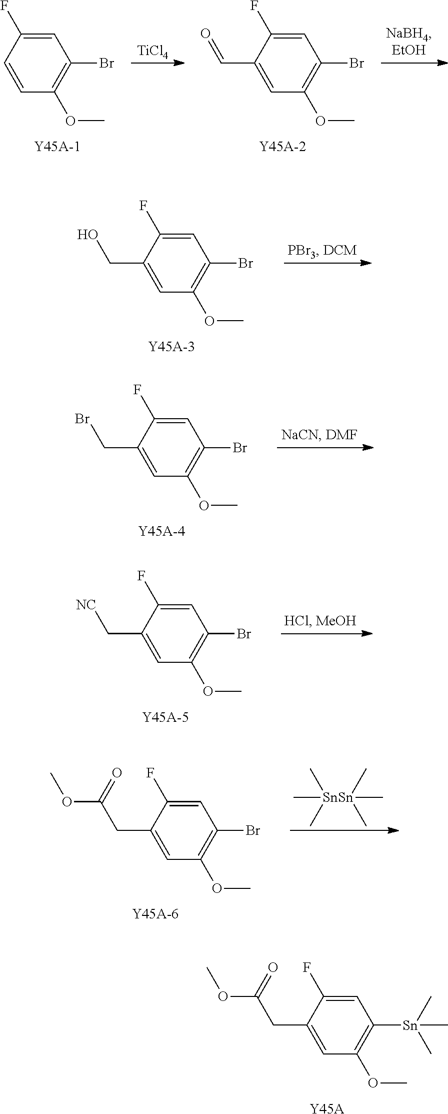

To a stirred solution of Y45A-1 (16 g, 78.43 mmol) was added TiCl₄ (17.12 mL) and stirred at rt for 30 min. Then the mixture was cooled to 0° C. and dichloro(methoxy)methane (13.85 mL) was dropwise into the mixture and stirred for 2 hrs. The mixture was diluted with DCM (50 mL), and slowly poured into crush ice. Then the mixture was extracted with DCM, then concentrated, the crude was used to next step directly.

To a stirred solution of Y45A-2 (9.6 g, 47.06 mmol) in EtOH (100 mL) was added NaBH₄ (1.79 g, 47.06 mmol) protionwise at 0° C. and stirred for about 30 min. Then NH₄Cl (aq., 30 mL) was added into the mixture slowly and the mixture was extracted with DCM and concentrated to give the crude product. The crude product was purified to afford the Y45A-3 (4 g, yield: 37.95%).

The preparation of Y45A-6 from Y45A-3 was similar to the procedure described in Method F. To a solution of Y45A-6 (1.3 g, 4.71 mmol) in dioxane (13 mL) was added Me₆Sn₂ (2.3 g, 7.06 mmol) and (PPh₃)₂PdCl₂ (659 mg, 0.942 mmol) under N₂ atmosphere. Then the mixture was heated to 110° C. for about 4 hs. Then water was added into the mixture and the solution was extracted with DCM. The crude product was purified to afford the Y45A (1.1 g, 64.7%).

### Synthesis of Building Block Y45B (Method Y)

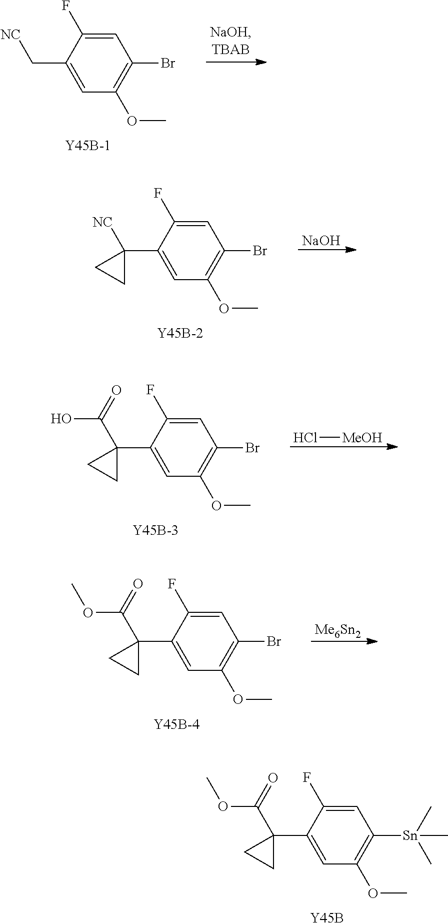

The preparation of Y45B-4 from Y45B-1 was similar to the procedure described in Method D. The preparation of Y45B from Y45B-4 was similar to the procedure described in in Method X.

### Synthesis of Building Block Y16B (Method Z)

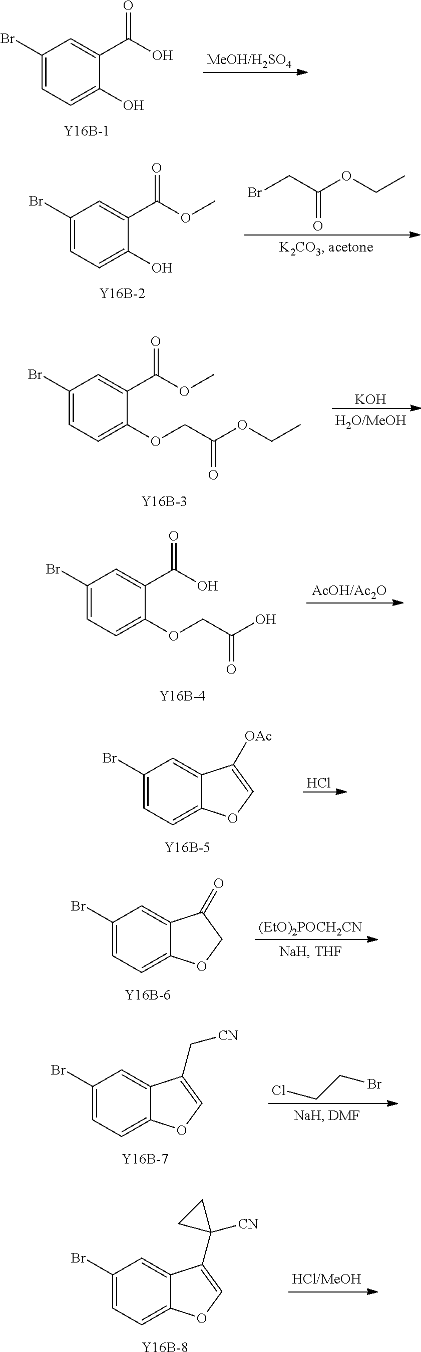

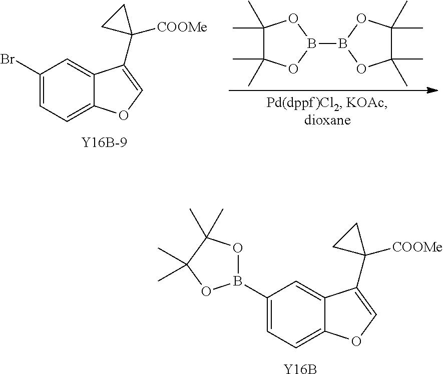

To a solution of acid Y16B-1 (100 g, 462.9 mmol) in MeOH (500 mL) was added concentrated sulfuric acid (40 mL) at rt. After addition, the solution was refluxed for 12 h. The mixture was poured into crashed ice (500 g), basified to pH=8 with solid NaHCO₃ and extracted with EtOAc. The combined organic layers were dried over Na₂SO₄ and filtered. The filtrate was concentrated to give Y16B-2 (106 g, yield: 99.5%) as a white solid.

A mixture of Y16B-2 (106 g, 460.9 mmol), ethyl 2-bromoacetate (93.5 g, 563.4 mmol) and K₂CO₃ (194.4 g, 1408.8 mmol) in acetone (1 L) was refluxed for 3 h. After cooling to rt, the mixture was partitioned between water and EtOAc. The organic layer was separated and the aqueous layer was extracted with EtOAc. The combined organic layers were dried over Na₂SO₄ and filtered. The filtrate was concentrated to give Y16B-3 (162 g, crude) as a yellowish oil, which solidified upon cooling to rt.

A mixture of Y16B-3 (162 g, crude) in MeOH (1 L) was added aq. KOH solution (8.53 mmol/mL, 300 mL) dropwise at rt. After addition, the mixture was stirred under reflux for 12 h. MeOH was removed under reduced pressure. The aqueous layer was extracted with EtOAc and the organic layer was discarded. The aqueous layer was poured into crashed ice (500 g) and acidified to pH=2 with concentrated HCl. The resulting precipitate was filtered and the filter cake was dried to give Y16B-4 (120 g, yield: 95% over two steps) as a white solid.

To a suspension of Y16B-4 (120 g, 438 mmol) in a mixed solvents of AcOH (500 mL) and Ac₂O (500 mL) was added sodium acetate (310 g, 3.78 mol) at rt. The mixture was refluxed for 24 h. The mixture was concentrated. The residue was dissolved in water and the mixture was extracted with DCM. The combined organic layers were dried and concentrated to afford a brown liquid, which was used in the next step without further purification.

To a solution of crude Y16B-5 in MeOH (800 mL) was added 1M HCl (800 mL) at rt. After addition, the mixture was refluxed for 4 h. MeOH was removed under reduced pressure. The resulting reddish solid was collected and further purified to give Y16B-6 (25 g, yield: 26.9% over two steps) as a reddish solid.

To a suspension of NaH (3.0 g, 75 mmol, 60% in mineral oil) in THF (100 mL) was added diethyl cyanomethyl phosphonate (12.0 g, 67.8 mmol) at 0° C. The mixture was stirred at rt for 1 h and then cooled to 5° C. A solution of Y16B-6 (14.0 g, 66 mmol) in THF (200 mL) was added dropwise. After addition, the mixture was stirred at rt for 2 h. To the mixture was added crashed ice (20 g). The mixture was partitioned between water and EtOAc. The organic layer was separated and the aqueous phase was extracted with EtOAc. The combined organic layers were dried and concentrated. The residue was purified to give Y16B-7 (8.5 g, yield: 54.8%) as a reddish solid.

To a solution of Y16B-7 (7.5 g, 31.9 mmol) in DMF (50 mL) was added portion wise of NaH (2.6 g, 65 mmol, 60% in mineral oil) at 0° C. After addition, the mixture was stirred at 0° C. for 1 h. Then 1-bromo-2-chloro-ethane (4.5 g, 31.9 mmol) was added dropwise at 0° C. The mixture was stirred at rt overnight. The mixture was treated with saturated aqueous NH₄Cl solution and extracted with EtOAc. The combined organic layers were dried over Na₂SO₄ and concentrated to give the crude product (10.0 g, crude) as brown oil, which was used in the next step directly.

A solution of Y16B-8 (10.0 g, crude) in HCl/MeOH (200 mL, 4 mmol/mL) was refluxed for 5 h. The solution was concentrated. The residue was poured into water and the mixture was extracted with EtOAc. The combined organic layers were dried and concentrated. The residue was purified to give Y16B-9 (2.6 g, yield: 27.7% over two steps) as a yellowish oil, which solidified while standing at rt.

The preparation of Y16B from Y16B-9 and bis(pinacolato)diboron was followed the similar procedure described in Method S.

### Synthesis of Building Block Y17A (Method AA)

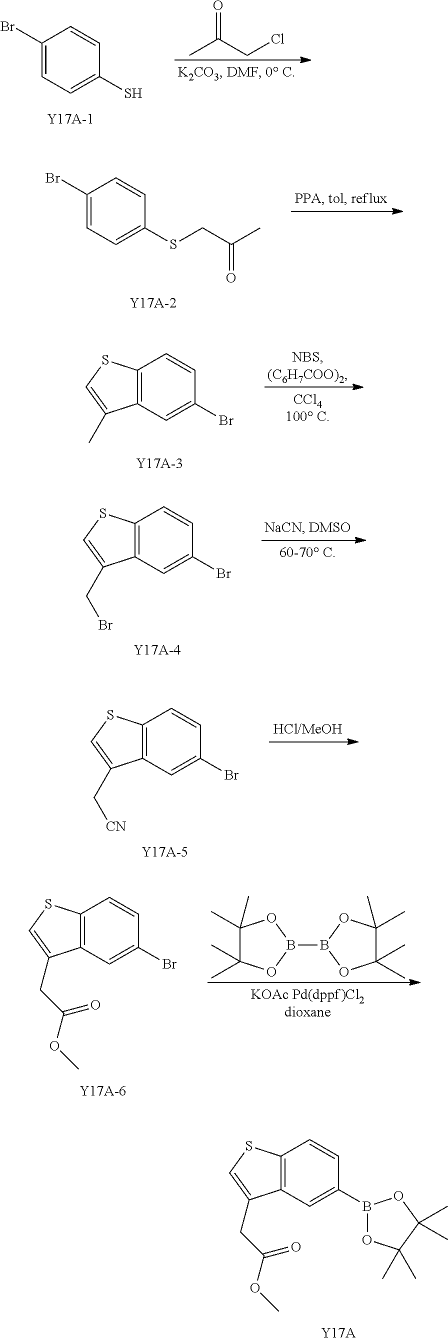

To a mixture of Y17A-1 (50 g, 266 mmol, 1 eq), 1-chloropropan-2-one (28.5 mL, 359 mmol, 1.35 eq) and K₂CO₃ (73.4 g, 532 mmol, 2 eq) in DMF (500 mL) was stirred at around 0° C. overnight. 1 L of water was added and extracted with EtOAc. The combined organic layer was washed with brine, dried over Na₂SO₄ and concentrated. The residue was purified to affor crude Y17A-2 (42 g, 64.6% yield) as a white solid.

A mixture of Y17A-2 (2.0 g) and PPA (30 g) in toluene (50 mL) was stirred at 120˜130° C. for 4 hrs. 50 ml of water was added and extracted with EtOAc. The combined organic layer was washed with brine, dried over Na₂SO₄ and concentrated. The residue was purified to afford Y17A-3 (1.5 g, 68% yield).

To a mixture of Y17A-3 (23.7 g, 104.87 mmol) and (C₆H₇COO)₂ (2.58 g, 10.487 mmol, 0.1 eq) in CCl₄ (100 mL) was added NBS (18.46 g, 104.87 mmol) and the mixture was stirred at 100-110° C. overnight. The mixture was concentrated and purified to afford Y17A-4 (22.5 g, 70.6% yield) as a white solid.

The preparation of the other intermediates and Y17A were followed the similar procedure described in Method T.

### Synthesis of Building Block Y19A (Method AB)

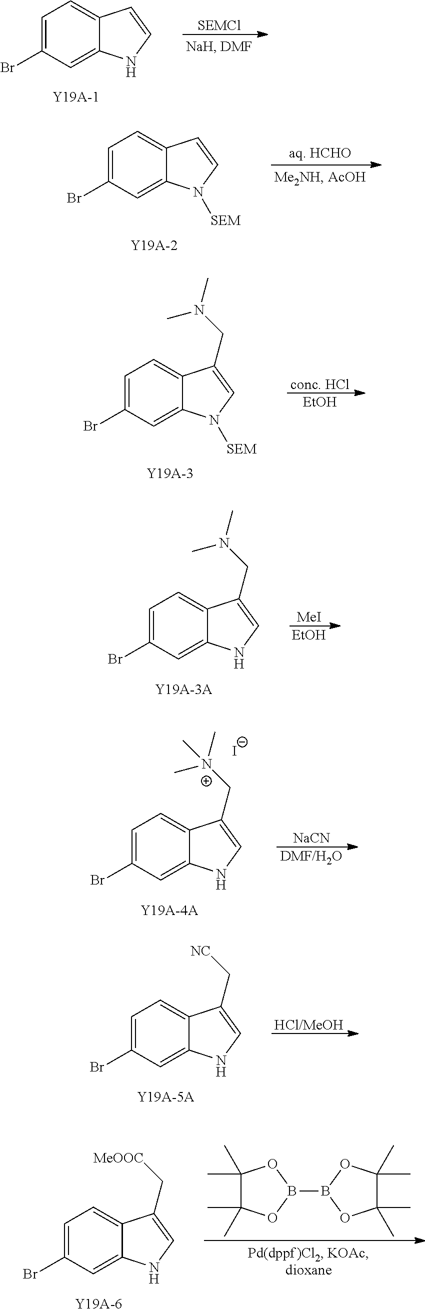

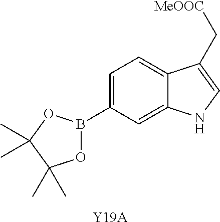 [SMILES: COC(=O)Cc1c[nH]c2cc(B3OC(C)(C)C(C)(C)O3)ccc12 ; "Y19A"]

To a solution of indole Y19A-1 (77 g, 400 mmol) in DMF (400 mL) was added NaH (16 g, 400 mmol, 60% in mineral oil) at 0° C. After addition, the mixture was stirred at 0° C. for 30 min and then SEMC1 (66.8 g, 400 mmol) was added dropwise at 0° C. The mixture was allowed to warm to rt and stirred overnight. The mixture was poured into crashed ice (500 g), followed by addition of 500 mL of saturated NH₄Cl solution. The mixture was extracted with EtOAc. The combined organic layers were dried and concentrated. The residue was purified to give Y19A-2 (58 g, yield: 44.6%) as a yellowish oil.

To aqueous dimethylamine solution (120 mL, 790 mmol, 33%) was added AcOH (150 mL) at 0° C. After addition, formaldehyde (80 mL, 1030 mmol, 36%) and Y19A-2 (23.2 g, 72 mmol) were added sequentially, while keeping the internal temperature at −5-0° C. The mixture was then stirred at 80° C. overnight. The mixture was then poured into water, extracted with EtOAc and the combined organic layers were concentrated. The residue was dissolved in CH₂Cl₂ (500 mL). The solution was washed with saturated aqueous NaOH solution, dried over Na₂SO₄ and concentrated to give Y19A-3 (21.3 g, yield: 77.5%) as a brown oil.

To a solution of Y19A-3 (17 g, 44.5 mmol) in ethanol (500 mL) was added concentrated HCl (150 mL) at 0° C. After addition, the mixture was refluxed overnight. The mixture was concentrated. The residue was dissolved in water, basified to pH=9 with solid K₂CO₃ and extracted with EtOAc. The combined organic layers were dried and concentrated to give Y19A-3A as a brown solid (10.2 g, yield: 91.2%), which was used for the next step directly.

To a solution of Y19A-3A (10.2 g, 40.5 mmol) in EtOH (150 mL) was added methyl iodide (36.6 g, 257.7 mmol) at rt. After addition, the mixture was stirred at rt overnight. The mixture was concentrated to a brown solid (18 g, crude), which was used for the next step directly.

The preparation of the other intermediates and Y19A were followed the similar procedure described in Method T.

### Synthesis of Building Block Y15B (Method AC)

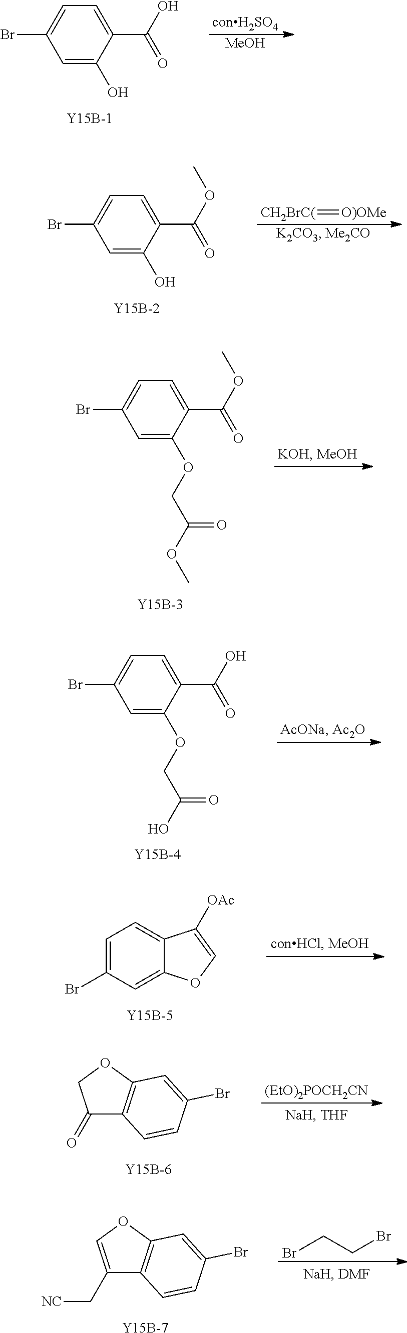

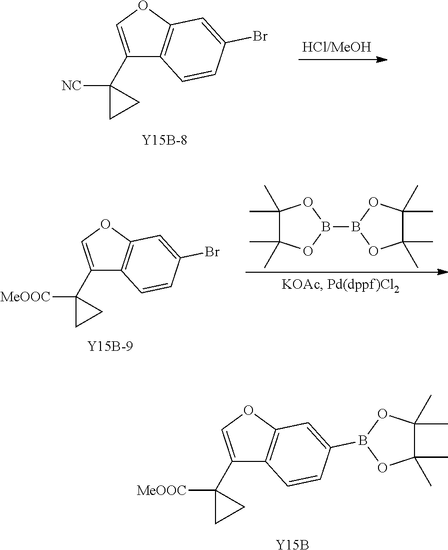

A stirred solution of Y15B-1 (20 g, 92.59 mmol), H₂SO₄ (2 mL) and CH₃OH (250 mL) was heated to 70° C. for 4 hs under argon. The solution was quenched with water, and extract with EtOAc, the combined organic layers was dried over Na₂SO₄, concentrated in vacuo, and purified to afford Y15B-2 (21 g, yield: 98.5%).

A stirred solution of Y15B-2 (410 g, 43.47 mmol), K₂CO₃ (9.06 g, 65.22 mmol) and methyl 2-bromoacetate (9.91 g, 65.22 mmol) in acetone was heated to 70° C. for 4 h under argon. The solution was quenched with water, extract with EtOAc, the water layers was acidified to pH˜3 and after standard workup to afford Y15B-3 (12.7 g, yield: 96.9%).

To a stirred solution of Y15B-3 (12.7 g, 42.05 mmol) in 200 mL of CH₃OH/H₂O (150 mL, v/v=4/1) was added KOH (4.7 g, 84.1 mmol). After the addition, the solution was worked up and adjusted pH to 2 with HCl (3N). The aqueous phase was extracted with DCM. The solid was filtered and the filtered was concentrated to afford Y15B-4 (9 g, crude)

To a solution of Y15B-4 (9 g, 32.84 mmol) in HOAc (80 mL) and Ac₂O (100 mL) was added NaOAc (7 g, 85.40 mmol). After stirring for 3 h at 150° C., the resulting mixture was dissolved in water, extracted with EA, dried over Na₂SO₄ and concentrated to give Y15B-5 (6 g, yield: 71.94%).

A solution of Y15B-5 (6 g, 23.6 mmol) in HCl (4 mL), MeOH (160 mL), and water (40 mL) was heated under reflux for 1.5 h, the reaction mixture was cooled to rt, dissolved in water and filtered to give Y15B-6 (3.5 g, yield: 70%).

To a stirred solution of (EtO)₂POCH₂CN (457 mg, 2.63 mmol) in 10 mL of THF was added NaH (103 mg, 2.63 mmol) at 0° C. The solution was stirred for 1 h and Y15B-6 (500 mg, 2.35 mmol) was added. The reaction mixture was stirred for 1.5 h. After quenched with water, the mixture was extracted with DCM. The combined organic layer was washed with brine, dried over Na₂SO₄, concentrated and purified to afford Y15B-7 (500 mg, yield: 90.25%).

To a stirred solution of Y15B-7 (500 mg, 2.13 mmol) in 10 mL of DMF was added NaH (127.6 mg, 3.19 mmol) at 0° C. The solution was stirred for 1 hour and BrCH₂CH₂Br (587 mg, 3.19 mmol) was added. The reaction mixture was stirred for 1.5 h. After quenched with water, the mixture was extracted with DCM. The combined organic layer was washed with brine, dried over Na₂SO₄, concentrated and purified to afford Y15B-8 (200 mg, yield: 36.04%).

A stirred solution of Y15B-8 (180 mg, 0.68 mmol) in HCl/CH₃OH (10 mL) was heated to 60° C. for 4 h under argon. The solution was quenched with water, and extract with EtOAc, the combined organic layers was dried over Na₂SO₄, concentrated and purified to afford Y15B-9 (170 mg, yield: 85%).

The preparation of Y15B from Y15B-9 was followed the similar procedure described in Method T.

### Synthesis of Building Block Y13B (Method AD)

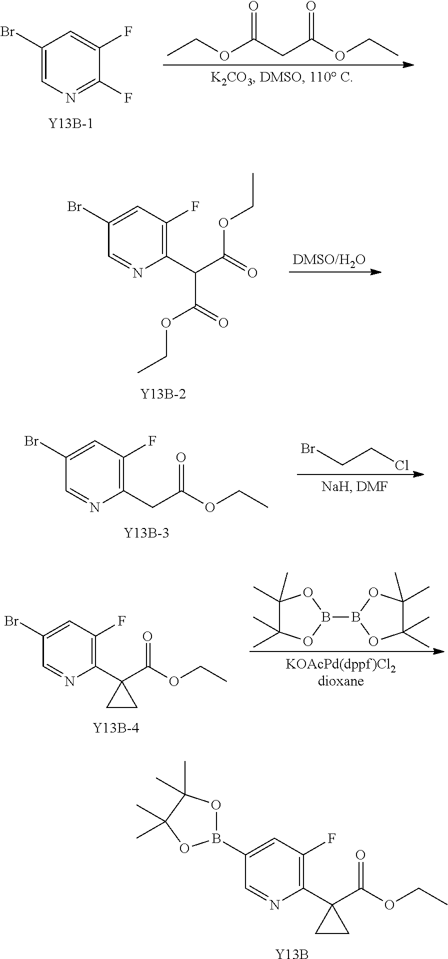

A mixture of Y13B-1 (2 g, 10.36 mmol, 1 eq), diethyl malonate (2.16, 13.47 mmol, 1.3 eq) and K₂CO₃ (4.29 g, 30.08 mmol, 3 eq) in DMF (20 mL) was stirred at 100-110° C. for 2 hrs. 80 mL of water was added and extracted with EtOAc. The combined organic layer was washed with brine, dried over Na₂SO₄ and concentrated to afford crude Y13B-2 (4.9 g, 142% yield).

A mixture of Y13B-2 (4.4 g, crude) in DMSO (20 mL)/H₂O was stirred at 160-165° C. for 4 hrs. 80 mL of water was added and extracted with EtOAc. The combined organic layer was washed with brine, dried over Na₂SO₄ and concentrated. The residue was purified to afford Y13B-3 (1.55 g, 42% yield) as a light yellow liquid.

To a mixture of Y13B-3 (3.6 g, 11.54 mmol, 1 eq.) in DMF (30 mL) was added NaH (1.15 g, 28.85 mmol, 2.5 eq.) in portions at 0° C. and stirred for 30 min. ClCH₂CH₂Br (1.97 g, 13.85 mmol, 1.2 eq) was added and stirred at 0° C. for 30 min. The mixture was warmed to rt and stirred overnight. LCMS showed the ratio of Y13B-3 and Y13B-4 was 22% and 26%. 70 mL of saturated aq. NH₄Cl was added and extracted with EtOAc. The combined organic layer was washed with brine, dried over Na₂SO₄ and concentrated in vacuum. The residue was purified to afford Y13B-4 (540 mg, 16% yield) as liquid.

The preparation of Y13B from Y13B-4 was followed the similar procedure described in Method T.

### Synthesis of Building Block Y20B (Method AE)

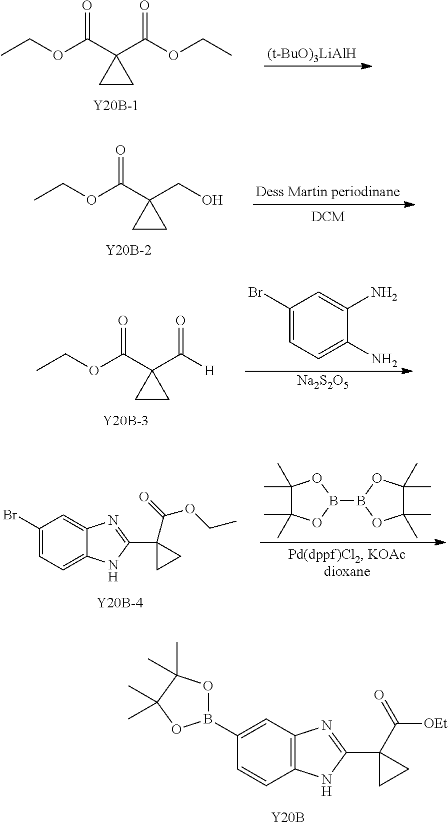

A suspension of lithium tri-tert-butoxyaluminum hydride (82 g, 0.32 mol) in 1000 mL of anhydrous THF was added to a solution of Y20B-1 (30 g, 0.16 mol) slowly. After addition, the mixture was stirred at 25° C. overnight. The mixture was quenched by addition of 2N HCl. Then the mixture was extracted with EA, the combined organic layer was washed with brine, dried and concentrated to afford Y20B-2 (20.6 g, yield 89%).

To a solution of Y20B-2 (14 g, 90 mmol) in 600 mL of DCM was added Dess-Martin periodinane (83.6 g, 190 mmol) in portions at 0° C. The mixture was stirred for 4 h at 0° C. The reaction mixture was quenched by the addition of Na₂S₂O₃ aqueous solution. Then NaHCO₃ aqueous solution was added to pH=7. The mixture was extracted with DCM. The combined organic layer was washed with brine, dried and concentrated. The residue was purified to give Y20B-3 (5.8 g, 42%) as a yellow liquid.

Y20B-3 (5.6 g, 40 mmol), Na₂S₂O₅ (9 g, 48 mmol) and 4-bromobenzene-1,2-diamine (7.4 g, 40 mmol) was dissolved in DMF (20 mL) and the solution was stirred at 130° C. for 3 h under N₂. The solution was then cooled to rt, diluted with water and extracted with EtOAc. The organic phase was washed with brine, dried over Na₂SO₄, concentrated and purified to Y20B-4 (4.3 g, yield 35%) as a white solid.

The preparation of Y20B from Y20B-4 was followed the similar procedure described in Method T.

### Synthesis of Building Block Y11B (Method AF)

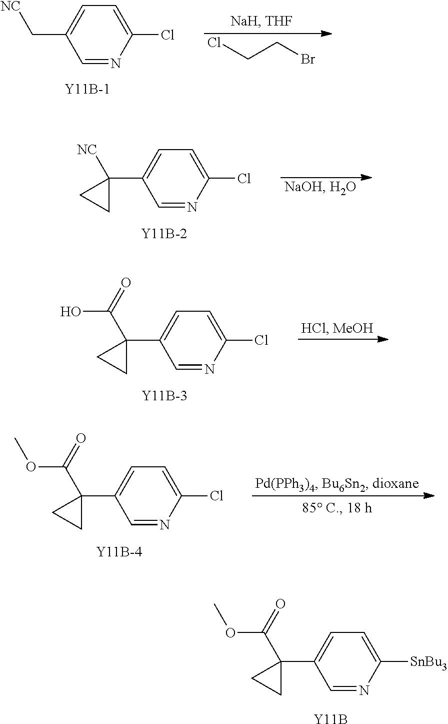

To a stirred solution of Y11B-1 (5 g, 32.8 mmol) in THF (50 mL) was added NaH (1.57 g, 65.5 mol) under nitrogen at 0° C. After stirring for 0.5 h at 0° C., ClCH₂CH₂Br (12.2 g, 0.085 mol) was added at rt overnight. The solution was quenched with aq. NH₄Cl and extracted with EtOAc. The combined organic layer was washed with brine, dried and evaporated. The residue was purified to afford Y11B-2 (3 g, yield: 52.0%).

To a stirred solution of Y11B-2 (3 g, 16.9 mol) in MeOH (20 mL) was added 35% NaOH (30 mL). Then the mixture was heated to 60° C. for overnight. MeOH was removed in vacuo. The residue was adjusted to pH=7 with HCl and extracted with EtOAc. The combined organic layer was washed with brine, dried and evaporated to afford Y11B-3 (3.0 g, yield: 90.9%).

To a stirred solution of Y11B-3 (6.5 g, 0.033 mol) in MeOH (150 mL) was added HCl (5 mL). Then the mixture was heated to reflux overnight. After concentrated, the residue was purified to afford Y11B-4 (3 g, yield: 43%).

To a mixture of Y11B-4 (300 mg, 4.7 mmol) in dioxane (5 mL) was added Bu₆Sn₂ (1.2 g, 2.1 mmol) and Pd(PPh₃)₄ (163.9 mg, 0.142 mmol) under argon. The reaction mixture was stirred overnight at 85° C. The mixture was filtered on silica gel and concentrated to afford compound Y11B (1 g, crude) which was used in the next step directly.

### Synthesis of Compound IT134

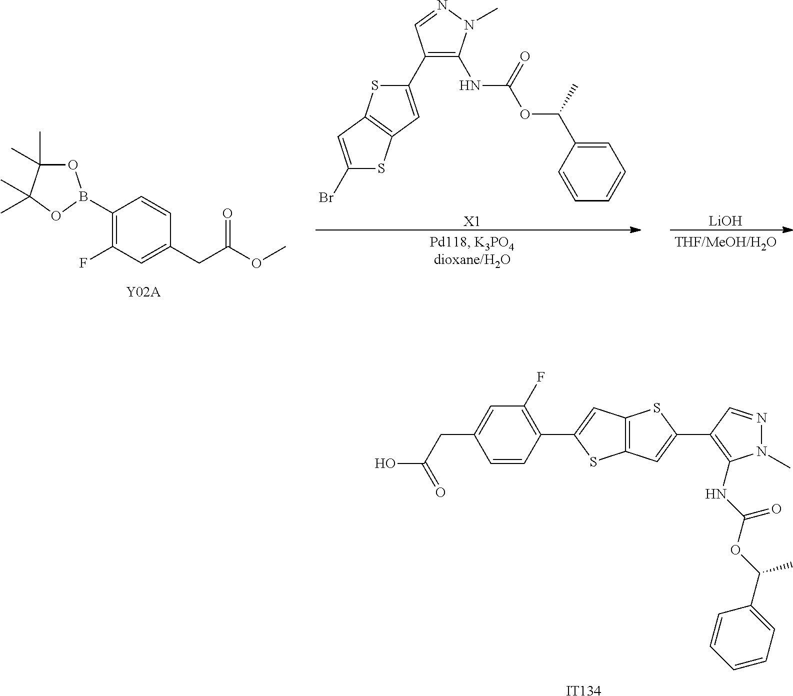

The mixture of Y02A (57.3 mg, 0.195 mmol), X1 (90 mg, 0.195 mmol), K₃PO₄ (83 mg, 0.389 mmol) and Pd-118 (6.3 mg, 0.0097 mmol) in dioxane/H₂O (15 mL, v/v=5/1) was microwaved at 80° C. under nitrogen for 15 min. After concentrated, the residue was partitioned between H₂O and DCM, the aqueous phase was extracted with DCM, and the combined organic layer was washed with brine, dried over Na₂SO₄, concentrated. The residue was purified to provide the intermediate methyl ester (50 mg, yield: 47.17%), which was dissolved in 6 mL of MeOH/H₂O/THF (v/v/v=1/1/1) and hydrolyzed by lithium hydroxide monohydrate (19.1 mg, 0.455 mmol) overnight. IT134 was obtained after standard workup and purification. ¹H NMR (DMSO-d₆, 400 MHz): δ 9.63 (s, 1H), 7.80-7.83 (d, J=8.4 Hz, 2H), 7.69-7.73 (t, J=8.0 Hz, 1H), 7.15-7.42 (m, 8H), 5.76-5.78 (q, J=6.6 Hz, 1H), 3.60-3.63 (m, 5H), 1.55-1.56 (d, J=6.4 Hz, 3H). MS (ESI) m/z (M+H)⁺536.1.

### Synthesis of Compound IT214

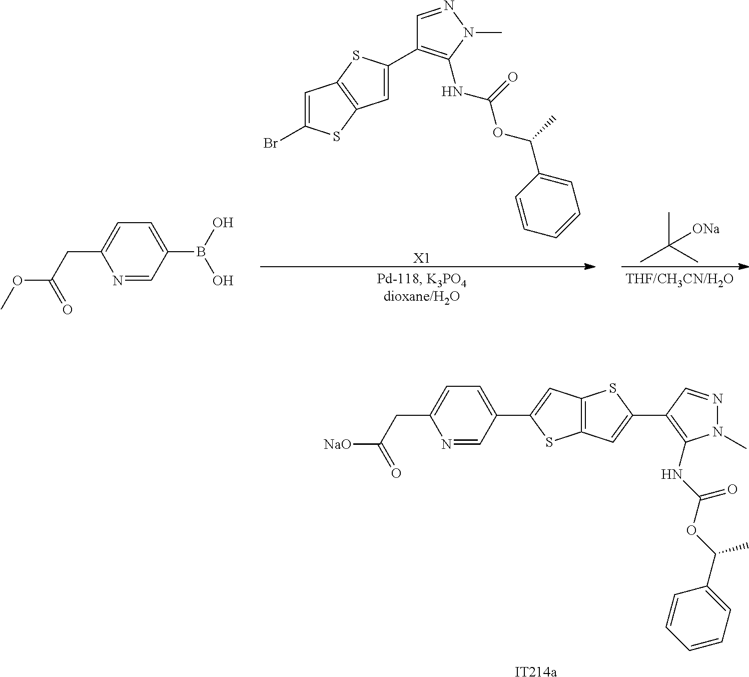

(6-(2-methoxy-2-oxoethyl)pyridin-3-yl)boronic acid was prepared according to the procedure described in Method F using 4-bromobenzoic acid in place of Y08A-1. The mixture of (6-(2-methoxy-2-oxoethyl)pyridin-3-yl)boronic acid (200 mg, 0.722 mmol), X1 (96.34 mg, 0.209 mmol), K₃PO₄ (86.9 mg, 0.418 mmol) and Pd-118 (6.64 mg, 0.01 mmol) in dioxane/H₂O (15 mL, v/v=5:1) was microwaved at 80° C. under nitrogen for 15 min. After concentrated, the residue was partitioned between H₂O and DCM, the aqueous phase was extracted with DCM, and the combined organic layer was washed with brine, dried over Na₂SO₄, concentrated. The residue was purified to provide the intermediate methyl ester (90 mg, yield: 83.33%), which was dissolved in CH₃CN (3 mL), THF (3 mL) and H₂O (1 mL) and hydrolyzed by t-BuONa (16.15 mg, 0.168 mmol) at rt for overnight. After concentrated, the mixture was freeze-dried to give sodium salt of IT214 (65 mg, yield: 71.04%)¹H NMR (DMSO-d₆, 400 MHz) δ 8.65 (d, J=1.2 Hz, 1H), 7.78-7.86 (m, 3H), 7.63 (s, 2H), 7.26-7.32 (m, 6H), 5.71-5.76 (d, J=6.4 Hz, 1H), 3.49 (s, 2H), 2.05 (s, 3H), 1.43-1.45 (d, J=6 Hz, 3H). MS (ESI) m/z (M+H)⁺ 519.1.

### Synthesis of Compound IT188

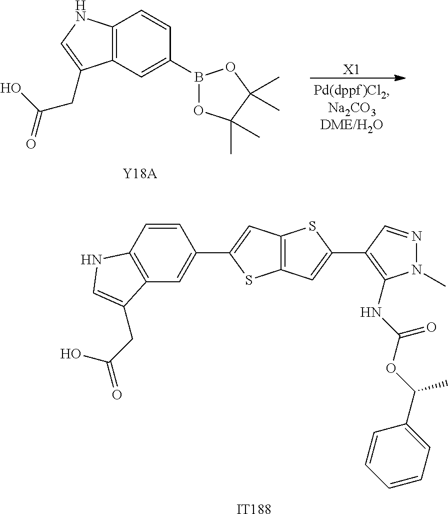 [SMILES: CC1(C)OB(c2ccc3[nH]cc(CC(=O)O)c3c2)OC1(C)C ; "X1" ; "Y18A" ; C[C@@H](OC(=O)Nc1c(-c2cc3sc(-c4ccc5[nH]cc(CC(=O)O)c5c4)cc3s2)cnn1C)c1ccccc1 ; "IT188"]

Y18A was prepared according to the procedure described in Method K. To a stirred mixture of compound Y18A (110 mg, 0.394 mmol), X1 (182 mg, 0.394 mmol), and Na₂CO₃ (124 mg, 1.17 mmol) in DME (5 mL) and H₂O (1 mL) was added Pd(dppf)₂Cl (28 mg, 0.039 mmol). The reaction mixture was flushed with Ar and stirred at 80° C. for 2 h. The mixture was extracted with EtOAc. The combined organic phase was dried over Na₂SO₄, and concentrated. The residue was purified by prep-HPLC to give IT188 (60 mg, yield: 27.6%). ¹H NMR (400 MHz, DMSO-d₆) δ 11.09 (s, 1H), 9.65 (s, 1H), 7.82 (s, 2H), 7.69 (s, 1H), 7.40-7.47 (m, 8H), 7.29-7.30 (m, 1H), 5.81-5.82 (d, J=6.4 Hz, 1H), 3.71 (s, 2H), 3.64 (s, 3H), 1.60 (d, J=6.4 Hz, 3H). MS (ESI) m/z (M+H)⁺ 557.0.

### Synthesis of Compound IT410

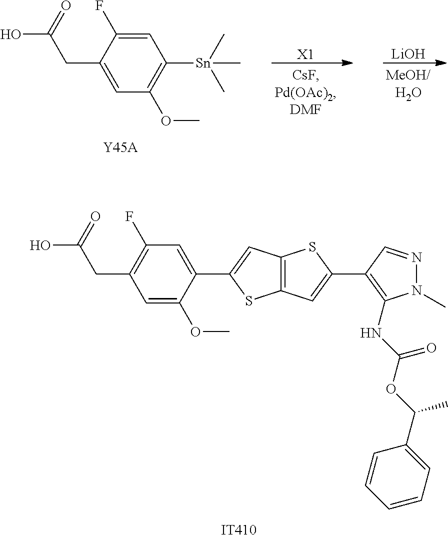 [SMILES: COc1cc(CC(=O)O)c(F)c[c]1[Sn]([CH3])([CH3])[CH3] ; "X1" ; "LiOH" ; "Y45A" ; COc1cc(CC(=O)O)c(F)cc1-c1cc2sc(-c3cnn(C)c3NC(=O)O[C@H](C)c3ccccc3)cc2s1 ; "IT410"]

Y45A was prepared according to Method X. The mixture of Y45A (235 mg, 0.64 mmol), X1 (150 mg, 0.32 mmol), CsF (49 mg, 0.32 mmol) and Pd(OAc)₂ (10 mg, 0.032 mmol) in DMF (5 mL) under nitrogen. Then the mixture was stirred at 80° C. for 15 hrs. After concentrated, the residue was added H₂O and extracted with DCM. The combined organic layer was washed with brine, dried over Na₂SO₄, concentrated. The residue was purified and redissovled in 5 mL of MeOH/H₂O (v/v=5/1) and subjected to hydrolysis by LiOH H₂O (13 mg, 0.311 mmol), and purified by Prep-HPLC to give IT410 (5.8 mg, yield: 19.74%). ¹H NMR (DMSO-d₆, 400 MHz): δ 9.64 (s, 1H), 7.96 (s, 1H), 7.831 (s, 1H), 7.63 (d, J=10.4 Hz, 1H), 7.37-7.46 (m, 6H), 7.17 (d, J=6.4 Hz, 1H), 5.80 (s, 1H), 3.93 (s, 3H), 3.65 (d, 5H), 3.02 (s, 3H).

Some exemplary compounds of Formula (III) were prepared following the similar procedure described herein and summarized in the Table 15 below.

TABLE 15

| | Building Block Y | | MS |
|---|---|---|---|
| Compound # | Preparation Method | Intermediate X | [M/Z (M + H)⁺] |
| IT119 | Method D | X1 | 544.0 |
| IT132 | Method A | X1 | 518.1 |
| IT120 | Method D | X3 | 561.0 |
| IT133 | Method A | X3 | 535.1 |
| IT149 | Method D | X2 | 545.1 |
| IT160 | Method A | X2 | 519.1 |
| IT169 | Method A | X1 | 532.1 |
| IT170 | Method A | X3 | 549.1 |
| IT171 | Method A | X2 | 533.1 |
| IT291 | Method B | X2 | 559.1 |
| IT260 | Method B | X1 | 558.1 |
| IT135 | Method C | X3 | 553.1 |
| IT159 | Method C | X2 | 537.1 |
| IT244 | Method D | X3 | 579.1 |
| IT245 | Method D | X1 | 562.1 |
| IT246 | Method D | X2 | 563.1 |
| IT140 | Method C | X1 | 552.0 |
| IT161 | Method C | X3 | 568.9 |
| IT168 | Method C | X2 | 553.1 |
| IT162 | Method C | X1 | 548.0 |
| IT163 | Method C | X3 | 565.1 |
| IT183 | Method C | X2 | 549.1 |
| IT264 | Method E | X1 | 574.1 |
| IT190 | Method C | X1 | 562.1 |
| IT191 | Method C | X2 | 563.1 |
| IT192 | Method C | X3 | 579.1 |
| IT156 | Method C | X3 | 571.0 |
| IT157 | Method C | X1 | 554.0 |
| IT158 | Method C | X2 | 555.1 |
| IT261 | Method D | X1 | 580.0 |
| IT262 | Method D | X2 | 581.0 |
| IT263 | Method D | X3 | 597.0 |
| IT164 | Method F | X1 | 536.0 |
| IT293 | Method F | X2 | 537.1 |
| IT294 | Method F | X3 | 553.0 |
| IT295 | Method D | X1 | 562.1 |
| IT296 | Method D | X2 | 563.1 |
| IT297 | Method D | X3 | 579.1 |
| IT211 | Method G | X1 | 554.0 |
| IT212 | Method G | X2 | 555.1 |
| IT265 | Method G | X3 | 571.1 |
| IT185 | Method F | X3 | 536.1 |
| IT215 | Method F | X2 | 520.1 |
| IT269 | Method H | X1 | 545.1 |
| IT270 | Method H | X2 | 546.1 |
| IT271 | Method H | X3 | 562.1 |
| IT275 | Method I | X1 | 575.1 |
| IT290 | Method C | X3 | 575.0 |
| IT240 | Method C | X1 | 555.9 |
| IT268 | Method J | X2 | 559.1 |
| IT283 | Method K | X3 | 574.1 |
| IT298 | Method K | X2 | 558.1 |
| IT180 | Method F | X1 | 533.1 |
| IT181 | Method F | X2 | 534.1 |
| IT182 | Method F | X3 | 550.1 |
| IT219 | Method L | X3 | 565.1 |
| IT220 | Method L | X2 | 549.1 |
| IT221 | Method L | X1 | 548.1 |
| IT165 | Method L | X1 | 552.0 |
| IT166 | Method L | X2 | 553.1 |
| IT167 | Method L | X3 | 569.0 |
| IT266 | Method B | X2 | 579.1 |
| IT267 | Method B | X3 | 595.0 |
| IT186 | Method L | X2 | 571.0 |
| IT187 | Method L | X1 | 570.0 |
| IT205 | Method L | X3 | 587.0 |
| IT284 | Method B | X1 | 596.1 |
| IT285 | Method B | X2 | 597.0 |
| IT286 | Method B | X3 | 613.0 |
| IT147 | Method M | X1 | 524.1 |
| IT148 | Method M | X3 | 541.0 |
| IT179 | Method M | X2 | 525.1 |
| IT272 | Method N | X1 | 550.0 |
| IT273 | Method N | X2 | 551.0 |
| IT274 | Method N | X3 | 567.0 |
| IT184 | Method O | X3 | 568.0 |
| IT189 | Method O | X2 | 552.1 |
| IT249 | Method O | X1 | 551.0 |
| IT206 | Method P | X1 | 585.8 |
| IT207 | Method P | X3 | 603.1 |
| IT216 | Method P&B | X3 | 629.1 |
| IT217 | Method P&B | X1 | 612.1 |
| IT218 | Method P&B | X2 | 613.1 |
| IT213 | Method Q | X3 | 619.0 |
| IT287 | Method Q&B | X1 | 628.1 |
| IT288 | Method Q&B | X2 | 629.1 |
| IT289 | Method Q&B | X3 | 645.0 |
| IT204 | Method R | X3 | 603.0 |
| IT222 | Method R | X1 | 586.1 |
| IT223 | Method R | X2 | 587.1 |
| IT299 | Method E | X2 | 613.1 |
| IT247 | Method E | X1 | 612.1 |
| IT248 | Method E | X3 | 629.0 |
| IT251 | Method R | X1 | 550.1 |
| IT252 | Method R | X3 | 567.1 |
| IT253 | Method R | X2 | 551.1 |
| IT241 | Method R | X3 | 567.0 |
| IT242 | Method R | X2 | 551.1 |
| IT243 | Method R | X1 | 550.1 |
| IT292 | Method R | X3 | 549.1 |
| IT250 | Method R | X2 | 533.1 |
| IT254 | Method R | X1 | 532.1 |
| IT317 | Method T | X3 | 585.0 |
| IT318 | Method T | X2 | 569.1 |
| IT319 | Method T | X1 | 568.1 |
| IT320 | Method D | X1 | 594.1 |
| IT321 | Method D | X2 | 595.1 |
| IT322 | Method D | X3 | 611.0 |
| IT323 | Method D | X1 | 558.1 |
| IT324 | Method D | X2 | 559.1 |
| IT325 | Method D | X3 | 575.0 |
| IT326 | Method F | X3 | 571.0 |
| IT327 | Method F | X1 | 554.0 |
| IT328 | Method F | X2 | 555.0 |
| IT329 | Method D | X1 | 574.1 |
| IT330 | Method D | X2 | 575.1 |
| IT331 | Method D | X3 | 591.0 |
| IT332 | Method J | X1 | 558.1 |
| IT333 | Method J | X3 | 575.1 |
| IT334 | Method Z | X1 | 584.1 |
| IT335 | Method Z | X2 | 585.1 |
| IT336 | Method AA | X2 | 575.1 |
| IT337 | Method E & F | X3 | 603.1 |
| IT338 | Method D | X3 | 593.0 |
| IT339 | Method D | X1 | 576.1 |
| IT340 | Method D | X2 | 577.1 |
| IT341 | Method D | X2 | 577.1 |
| IT342 | Method D | X1 | 576.1 |
| IT343 | Method D | X3 | 593.0 |
| IT346 | Method D | X3 | 597.0 |
| IT347 | Method D | X1 | 580.0 |
| IT348 | Method D | X2 | 581.0 |
| IT349 | Method F | X1 | 578.1 |
| IT350 | Method F | X2 | 579.1 |
| IT351 | Method F | X3 | 595.1 |
| IT353 | Method AA | X1 | 574.0 |
| IT354 | Method AA | X3 | 591.1 |
| IT357 | Method E | X2 | 575.1 |
| IT358 | Method I | X2 | 576.1 |
| IT359 | Method I | X3 | 592.1 |
| IT360 | Method E | X3 | 591.1 |
| IT361 | Method B | X3 | 575.1 |
| IT362 | Method E & F | X1 | 586.1 |
| IT363 | Method E & F | X2 | 587.1 |
| IT364 | Method D & F | X1 | 580.1 |
| IT365 | Method D & F | X2 | 581.1 |
| IT366 | Method D & F | X3 | 597.1 |
| IT367 | Method B | X1 | 578.1 |
| IT369 | Method C | X2 | 559.1 |
| IT370 | Method AA & D | X1 | 600.1 |
| IT372 | Method AA & D | X2 | 601.1 |
| IT373 | Method AA & D | X3 | 617.1 |
| IT376 | Method AB | X2 | 574.1 |
| IT377 | Method AB | X1 | 557.1 |
| IT378 | Method AB | X2 | 558.1 |
| IT379 | Method S | X1 | 612.1 |
| IT380 | Method S | X2 | 613.1 |
| IT381 | Method S | X3 | 629.0 |
| IT382 | Method X | X3 | 583.0 |
| IT383 | Method U | X1 | 602.1 |
| IT384 | Method U | X3 | 619.0 |
| IT385 | Method D | X1 | 628.2 |
| IT386 | Method D | X2 | 629.1 |
| IT387 | Method D | X3 | 645.1 |
| IT389 | Method D | X1 | 578.1 |
| IT390 | Method D | X2 | 579.1 |
| IT391 | Method D | X3 | 595.1 |
| IT392 | Method AD | X1 | 563.2 |
| IT393 | Method AD | X2 | 564.2 |
| IT394 | Method AD | X3 | 580.1 |
| IT395 | Method AC | X1 | 584.2 |
| IT396 | Method AC | X2 | 585.2 |
| IT397 | Method AC | X3 | 601.1 |
| IT410 | Method X | X1 | 566.0 |
| IT411 | Method Y | X1 | 592.1 |
| IT412 | Method Y | X3 | 609.0 |
| IT413 | Method D | X3 | 621.0 |
| IT414 | Method D | X1 | 604.1 |
| IT415 | Method D | X2 | 605.1 |
| IT416 | Method U | X2 | 603.0 |
| IT421 | Method AE | X3 | 601.1 |
| IT422 | Method AE | X2 | 585.2 |
| IT426 | Method AF | X3 | 562.1 |
| IT427 | Method AE | X1 | 584.1 |
| IT433 | Method V | X3 | 613.0 |
| IT441 | Method W | X1 | 622.1 |
| IT442 | Method W | X2 | 623.1 |
| IT443 | Method V | X2 | 597.0 |
| IT445 | Method V | X1 | 596.0 |
| IT458 | Method W | X3 | 639.0 |
| IT480 | Method Z | X3 | 601.1 |

IT235 was prepared by the Suzuki-coupling of methyl 1-(4-(4,4,5,5-tetramethyl-1,3,2-dioxaborolan-2-yl)phenyl)cyclobutanecarboxylate and (R)-1-phenylethyl (5-(4-bromophenyl)-3-methylisoxazol-4-yl)carbamate using Pd-118 as catalyst, followed by standard LiOH hydrolysis. ¹H NMR (DMSO-d₆, 400 MHz): δ 8.92 (s, 1H), 7.78 (m, J=8.8 Hz, 4H), 7.70 (d, J=8.4 Hz, 2H), 7.30-7.44 (m, 7H), 5.76-5.81 (q, J=6.4 Hz, 1H), 2.78-2.80 (m, 2H), 2.27-2.50 (m, 2H), 2.15 (s, 3H), 1.98-2.05 (m, 1H), 1.85-1.88 (m, 1H), 1.52 (d, J=6.4 Hz, 3H) MS (ESI) m/z (M+H)⁺ 497.2.

IT276 was prepared by the Suzuki-coupling of ethyl 3-(4,4,5,5-tetramethyl-1,3,2-dioxaborolan-2-yl)cyclopent-2-enecarboxylate and X3 following the similar procedure described in the synthesis of IT188, followed by Pd/C hydrogenation and LiOH hydrolysis to afford IT276 as the final product. ¹H NMR (DMSO-d₆, 400 MHz): δ 8.91 (s, 1H), 7.64-7.69 (m, 1H), 7.31-7.38 (m, 5H), 7.24 (s, 1H), 5.78 (q, J=6.4 Hz, 1H), 3.43-3.48 (m, 1H), 2.89-2.93 (m, 1H), 2.53-2.54 (m, 1H), 2.42-2.47 (m, 1H), 2.25 (s, 3H), 2.15-2.23 (m, 1H), 1.98-2.00 (m, 1H), 1.91-1.95 (m, 1H), 1.78-1.89 (m, 1H), 1.52 (br, 3H). MS (ESI) m/z (M+H)⁺ 513.1.

### Synthesis of Compound IT142

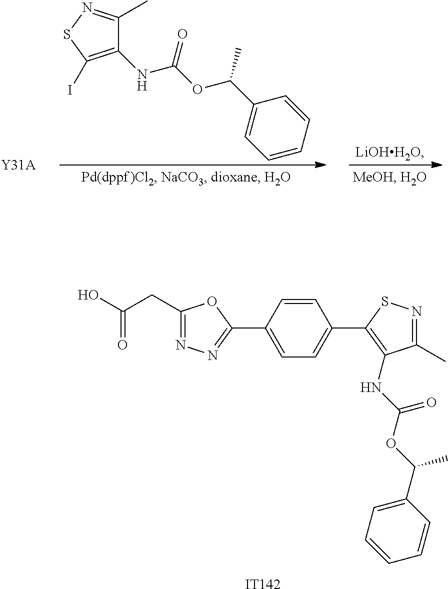 [SMILES: "Y31A" ; Cc1nsc(I)c1NC(=O)O[C@H](C)c1ccccc1 ; Cc1nsc(-c2ccc(-c3nnc(CC(=O)O)o3)cc2)c1NC(=O)O[C@H](C)c1ccccc1 ; "IT142"]

Y31A was prepared using the procedures described in Method P. To a stirred solution of Y31A (150 mg, 0.42 mmol), (R)-1-phenylethyl (5-iodo-3-methylisothiazol-4-yl)carbamate (195 mg, 0.50 mmol), Na₂CO₃ (89 mg, 0.84 mmol) in dioxane/H₂O (18 mL, 5/1) was added Pd(dppf)Cl₂ (61 mg, 0.084 mmol) under nitrogen. Then the mixture was heated to 100° C. for 4 h. After concentrated, H₂O was added and extracted with EtOAc. The combined organic layers were washed with brine, dried and evaporated. The residue was purified to provide the intermediate ester (110 mg, yield: 53.2%), which was dissolved in 9.6 mL of MeOH/H₂O (v/v=5/1) and hydrolyzed by lithium hydroxide (47 mg, 1.12 mmol) according to the standard procedure. After workup, residue was purified by Prep-HPLC and freeze-dried to provide IT142 (48.1 mg, yield: 46.3%). MS (ESI) m/z (M+H)⁺ 465.2. ¹H NMR (Methanol-d₄, 400 MHz): δ 8.03 (d, J=8 Hz, 2H), 7.63 (d, J=7.2 Hz, 2H), 7.25-7.35 (m, 5H), 5.73 (br, 1H), 4.16 (s, 2H), 2.35 (s, 3H), 1.55 (d, J=6.4 Hz, 3H).

IT143 was prepared following the similar procedure described in the synthesis of IT142 using (R)-1-phenylethyl (4-iodo-1-methyl-1H-1,2,3-triazol-5-yl)carbamate in place of the isothiazole analog. MS (ESI) m/z (M+H)⁺ 449.2. ¹H NMR (Methanol-d₄, 400 MHz): δ 8.04 (d, J=8.4 Hz, 2H), 7.88 (d, J=8 Hz, 2H), 7.36-7.40 (m, 5H), 5.71 (br, 1H), 4.14 (s, 2H), 3.90 (s, 3H), 1.60 (d, J=6.4 Hz, 3H).

### Synthesis of Compound IT176

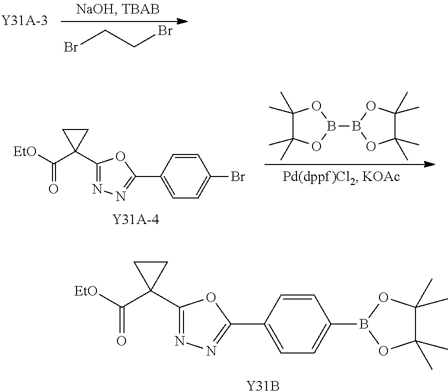

To a stirred solution of Y31A-3 (4 g, 12.86 mmol), 1,2-dibromoethane (4.35 g, 23.2 mmol), TBAB (1.03 g, 3.2 mmol) in DCM (150 mL) was added 50% NaOH (10 g, 0.25 mol) under nitrogen at rt and stirred for 8 h at rt. H₂O was added and extracted with DCM. The combined organic layer was washed with brine, dried and evaporated. The residue was purified to provide Y31A-4 (2.1 g, yield: 48.6%). Y31B was prepared by reacting Y31A-4 (1.4 g, 4.15 mmol) with bis(pinacolato)diboron (1.3 mg, 4.99 mmol) following the similar procedure described in the synthesis of Y01A (1.4 g, yield: 87.5%).

IT176 was prepared by Suzuki Coupling of Y31B with (R)-1-phenylethyl (5-iodo-3-methylisothiazol-4-yl)carbamate and subsequent LiOH hydrolysis following the same procedure described in the synthesis of IT142. MS (ESI) m/z (M+H)⁺ 491.2. ¹H NMR (Methanol-d₄, 400 MHz): δ 8.03 (d, J=7.6 Hz, 2H), 7.64 (d, J=7.6 Hz, 2H), 7.28-7.36 (m, 5H), 5.73 (br, 1H), 2.35 (s, 2H), 1.79 (s, 2H), 1.69 (d, J=3.2 Hz, 3H), 1.55 (d, J=5.2 Hz, 3H).

IT175 was prepared following the similar procedure described in the synthesis of IT176 using (R)-1-phenylethyl (4-iodo-1-methyl-1H-1,2,3-triazol-5-yl)carbamate in place of the isothiazole analog. MS (ESI) m/z (M+H)⁺ 475.2. ¹H NMR (Methanol-d₄, 400 MHz): δ 8.04 (d, J=8.4 Hz, 2H), 7.88 (d, J=8.4 Hz, 2H), 7.35-7.40 (m, 5H), 5.82 (br, 1H), 3.93 (s, 2H), 1.77-1.80 (m, 2H), 1.68-1.70 (m, 5H).

IT200 was prepared following the similar procedure described in the synthesis of IT176 using ethyl 1-(5-(4-bromophenyl)-1,3,4-thiadiazol-2-yl)cyclopropanecarboxylate in place of Y31A-4. MS (ESI) m/z (M+H)⁺ 507.1. ¹H NMR (Methanol-d₄, 400 MHz): δ 7.95 (d, J=7.6 Hz, 2H), 7.59 (d, J=8.4 Hz, 2H), 7.31-7.37 (m, 5H), 5.52 (br, 1H), 2.34 (s, 3H), 2.01-2.04 (m, 2H), 1.95-1.98 (m, 2H), 1.56 (d, J=6.0 Hz, 3H).

IT208 was prepared following the similar procedure as IT200 using (R)-1-phenylethyl (4-iodo-1-methyl-1H-1,2,3-triazol-5-yl)carbamate in place of the isothiazole analog. MS (ESI) m/z (M+H)⁺ 491.1. ¹H NMR (Methanol-d₄, 400 MHz): δ 7.94 (d, J=7.2 Hz, 2H), 7.83 (s, 2H), 7.31-7.4 (m, 5H), 5.83 (br, 1H), 3.92 (s, 3H), 2.0 (m, 2H), 1.95 (m, 2H), 1.62 (s, 3H).

IT239 was prepared following the similar procedure for the synthesis of IT175 using methyl 1-(2-(4-(4,4,5,5-tetramethyl-1,3,2-dioxaborolan-2-yl)phenyl)thiazol-5-yl)cyclopropanecarboxylate in place of Y31B. MS (ESI) m/z (M+H)⁺ 490.1. ¹H NMR (Methanol-d₄, 400 MHz): δ 7.90 (d, J=8.0 Hz, 2H), 7.79 (d, J=8.0 Hz, 2H), 7.70 (s, 1H), 7.40 (m, 4H), 5.83 (br, 1H), 3.92 (s, 3H), 1.80-1.83 (m, 2H), 1.62 (s, 3H), 1.47-1.50 (m, 2H).

IT278 was prepared following the similar procedure for the synthesis of IT175 using methyl 1-(5-(4-(4,4,5,5-tetramethyl-1,3,2-dioxaborolan-2-yl)phenyl)thiazol-2-yl)cyclopropanecarboxylate in place of Y31B. MS (ESI) m/z (M+H)⁺ 490.2. ¹H NMR (Methanol-d₄, 400 MHz): δ 7.96 (s, 1H), 7.64-7.74 (m, 4H), 7.42 (br, 5H), 5.86 (br, 1H), 3.93 (s, 3H), 1.82 (m, 2H), 1.92 (m, 2H), 1.65 (br, 3H).

IT144 was prepared by the Suzuki-Coupling of ethyl 5-(4,4,5,5-tetramethyl-1,3,2-dioxaborolan-2-yl)thiophene-2-carboxylate with (R)-1-phenylethyl (5-(4-bromophenyl)-3-methylisothiazol-4-yl)carbamate using the standard Pd(dpppf)Cl₂ coupling procedure followed by standard LiOH hydrolysis. MS (ESI) m/z (M+H)⁺ 465.1. ¹H NMR (Methanol-d₄, 400 MHz): δ 7.71-7.8 (m, 3H), 7.52-7.53 (m, 3H), 7.33-7.40 (m, 4H), 7.02-7.20 (br, 1H), 5.78-5.79 (br, 1H), 2.35 (s, 3H), 1.58-1.59 (m, 3H).

IT203 was prepared by the Suzuki-Coupling of ethyl 5-(4-(4,4,5,5-tetramethyl-1,3,2-dioxaborolan-2-yl)phenyl)thiophene-2-carboxylate with (R)-1-phenylethyl (4-iodo-1-methyl-1H-1,2,3-triazol-5-yl)carbamate using the standard Pd(dpppf)Cl₂ coupling procedure followed by LiOH hydrolysis. MS (ESI) m/z (M+H)⁺ 449.1. ¹H NMR (Methanol-d₄, 400 MHz): δ 7.73-7.79 (m, 5H), 7.45-7.57 (m, 6H), 5.86 (br, 1H), 3.93 (s, 3H), 1.65 (br, 3H).

IT141 was prepared by the Suzuki-Coupling of (R)-1-phenylethyl (5-(4-bromophenyl)-3-methylisothiazol-4-yl)carbamate with Y27A in THF/H₂O catalyzed by Pd(PPh₃)₄ and K₂CO₃ followed by standard LiOH hydrolysis. MS (ESI) m/z (M+H)⁺ 479.1. ¹H NMR (Methanol-d₄, 400 MHz): δ 7.33-7.61 (m, 9H), 6.98-7.19 (m, 2H), 5.78-5.80 (m, 1H), 3.89 (s, 2H), 2.34 (s, 3H), 1.58-1.59 (d, J=3.2 Hz, 3H).

IT178 was prepared following the similar procedure described in the synthesis of IT141 using (R)-1-phenylethyl (4-(4-bromophenyl)-1-methyl-1H-1,2,3-triazol-5-yl)carbamate in place of the isothiazolyl carbamate analog. MS (ESI) m/z (M+H)⁺ 463.1. ¹H NMR (Methanol-d₄, 400 MHz): δ 7.56-7.66 (m, 4H), 7.26-7.41 (m, 6H), 6.92-6.93 (br, 1H), 3.88 (s, 3H), 3.83 (s, 2H), 1.59 (br, 3H).

IT236 was prepared by the Suzuki-Coupling of (R)-1-phenylethyl (4-iodo-1-methyl-1H-1,2,3-triazol-5-yl)carbamate with Y27B using the standard Pd(dpppf)Cl₂ coupling procedure followed by LiOH hydrolysis. MS (ESI) m/z (M+H)⁺ 489.1. ¹H NMR (Methanol-d₄, 400 MHz): δ 7.68 (m, 2H), 7.60-7.62 (m, 2H), 7.45 (m, 5H), 7.25-7.26 (d, J=4 Hz, 1H), 6.96-6.97 (d, J=4 Hz, 1H), 5.85 (br, 1H), 3.93 (s, 3H), 1.72-1.74 (m, 2H), 1.64 (m, 3H), 1.38-1.41 (m, 2H).

### Example 42

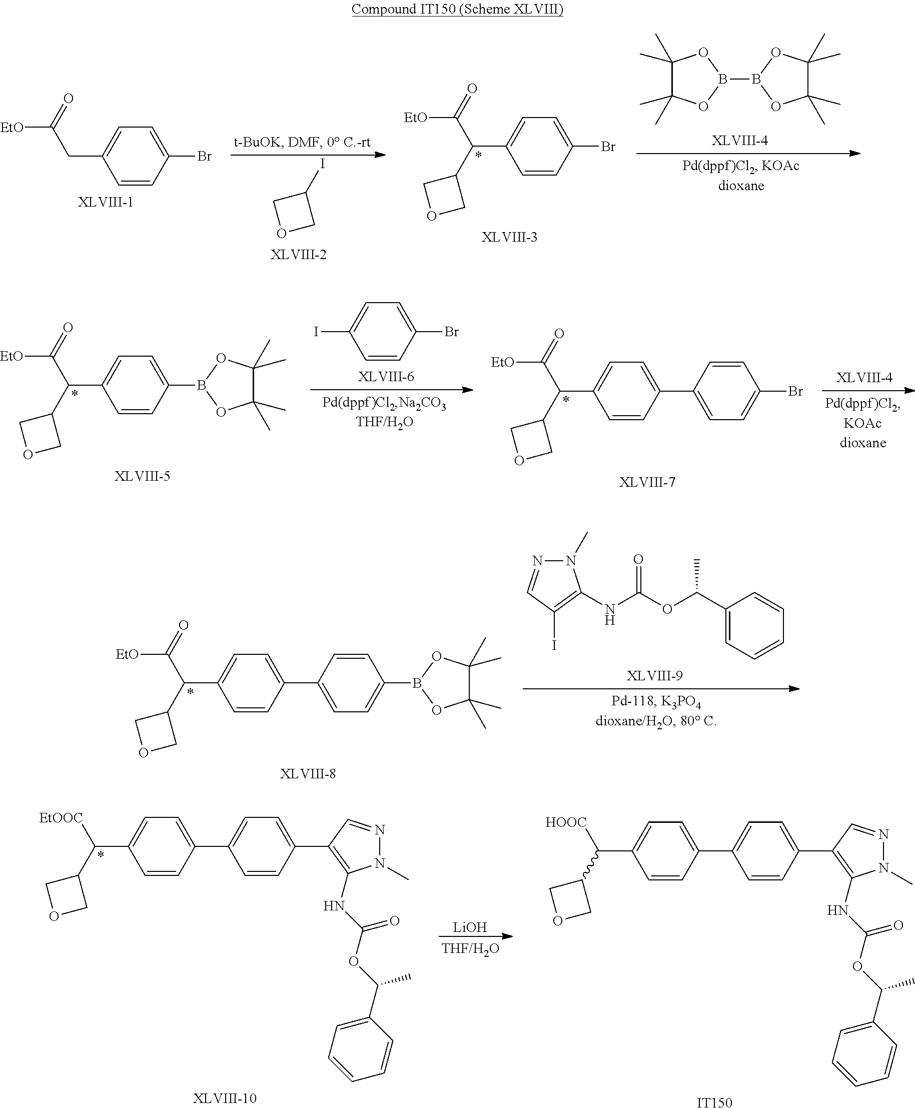

To a solution of XLVIII-1 (8 g, 32.92 mmol) in DMF (180 mL) was added t-BuOK (5.53 g, 49.38 mmol) at 0° C. The mixture was stirred for 30 min at 0° C. Then compound 2 (6.66 g, 36.20 mmol) was added in one portion. The resulting mixture was stirred for 8 h at rt. The reaction was quenched with water and the mixture was extracted with EtOAc. The combined organic layers were washed with brine, dried over anhydrous sodium sulfate, filtered and concentrated. The residue was purified to give XLVIII-3 (6.0 g, yield: 61%) as a light yellow oil.

A mixture of XLVIII-3 (5.5 g, 18.39 mmol), XLVIII-4 (5.6 g, 22.07 mmol), KOAc (3.6 g, 36.78 mmol) and Pd(dppf)Cl₂ (0.67 g, 0.92 mmol) in dioxane (60 mL) was stirred at 90° C. for 6 h under N₂. After cooling to rt, the mixture was diluted with water and the mixture was extracted with EtOAc. The combined organic layers were washed with brine, dried over anhydrous Na₂SO₄, and concentrated in vacuo. The residue was purified to give XLVIII-5 (5 g, yield: 79%) as a yellowish solid.

To a solution of XLVIII-5 (5.0 g, 14.45 mmol) in THF/H₂O (120 mL, V/V=5/1) was added Na₂CO₃ (3.06 g, 28.9 mmol), XLVIII-6 (4.1 g, 14.45 mmol) and Pd(dppf)Cl₂ (0.53 g, 0.72 mmol). The mixture was purged with nitrogen three times and then stirred at 85° C. for 8 h. After cooling to rt, the mixture was diluted with water and the mixture was extracted with EtOAc. The combined organic layers were washed with brine (100 mL), dried over anhydrous Na₂SO₄, and concentrated in vacuo. The residue was purified to give XLVIII-7 (3.5 g, yield: 65%) as a yellowish solid.

A mixture of XLVIII-7 (1.5 g, 4 mmol), XLVIII-4 (1.52 g, 6 mmol), KOAc (0.78 g, 8 mmol) and Pd(dppf)Cl₂ (0.15 g, 0.2 mmol) in dioxane (50 mL) was stirred at 85° C. for 18 h under N₂. After cooling to rt, the mixture was diluted with water and the mixture was extracted with EtOAc. The combined organic layers were washed with brine, dried over anhydrous Na₂SO₄, and concentrated in vacuo. The residue was purified to give XLVIII-8 (1 g, yield: 59%) as yellowish solid.

A mixture of XLVIII-8 (426 mg, 1.01 mmol), XLVIII-9 (250 mg, 0.67 mmol), K₃PO₄ (284 mg, 1.34 mmol) and Pd-118 (22 mg, 0.033 mmol) in dioxane/H₂O (12 mL, V/V=5/1) was stirred at 80° C. for 2 h. After cooling to rt, the mixture was diluted with water and the mixture was extracted with EtOAc. The combined organic layers were washed with brine, dried over anhydrous Na₂SO₄, and concentrated in vacuo. The residue was purified to give XLVIII-10 (280 mg, yield: 77%) as a yellowish solid. After standard LiOH hydrolysis, IT150 was obtained as a white solid. ¹H NMR (CDCl3, 400 MHz): δ 7.60 (bs, 1H), 7.57-7.54 (m, 2H), 7.49 (d, J=8.8 Hz, 2H), 7.36-7.33 (m, 9H), 6.28 (s, 1H), 5.89 (m, 1H), 4.96 (t, J=7.2 Hz, 1H), 4.64 (t, J=7.2 Hz, 1H), 4.55 (t, J=6.4 Hz, 1H), 4.34 (t, J=7.2 Hz, 1H), 4.14 (d, J=11.2 Hz, 1H), 3.77 (m, 4H), 1.61 (bs, 3H).

IT151 was prepared as a white solid following the similar procedure described in the synthesis of IT150 using bromocyclobutane in place of XLVIII-2. ¹H NMR (CDCl₃, 400 MHz): δ 7.73 (brs, 1H), 7.56-7.39 (m, 11H), 7.20-7.01 (m, 2H), 6.28 (brs, 1H), 5.88 (brs, 1H), 3.79 (s, 3H), 3.65 (d, J=10.0 Hz, 1H), 3.11-2.98 (m, 1H), 2.34-2.23 (m, 1H), 1.98-1.82 (m, 4H), 1.68-1.59 (m, 1H), 1.66 (s, 3H).

80 mg of IT151 was separated by SFC to give IT151A (20 mg) and IT151B (10 mg) both as white solids. IT151A: 1H NMR (DMSO-d₆, 400 MHz): δ 9.69 (s, 1H), 7.80 (s, 1H), 7.63-7.54 (m, 2H), 7.53-7.47 (m, 3H), 7.46-7.39 (m, 3H), 7.39-7.30 (m, 3H), 7.28-7.11 (m, 1H), 5.77 (brs, 1H), 3.73 (s, 3H), 3.29 (d, J=8.8 Hz, 1H), 2.92-2.83 (m, 1H), 2.11-2.04 (m, 1H), 1.81-1.67 (m, 2H), 1.76 (s, 3H), 1.60-1.52 (m, 2H), 1.51-1.42 (m, 1H). IT151B: 1H NMR (DMSO-d₆, 400 MHz): δ 9.63 (s, 1H), 7.80 (s, 1H), 7.62-7.47 (m, 6H), 7.46-7.30 (m, 6H), 7.24-7.12 (m, 1H), 5.78 (s, 1H), 3.63 (s, 3H), 2.92-2.86 (m, 1H), 2.15-2.08 (m, 1H), 1.85-1.66 (m, 2H), 1.75 (s, 3H), 1.62-1.47 (m, 4H).

### Example 43

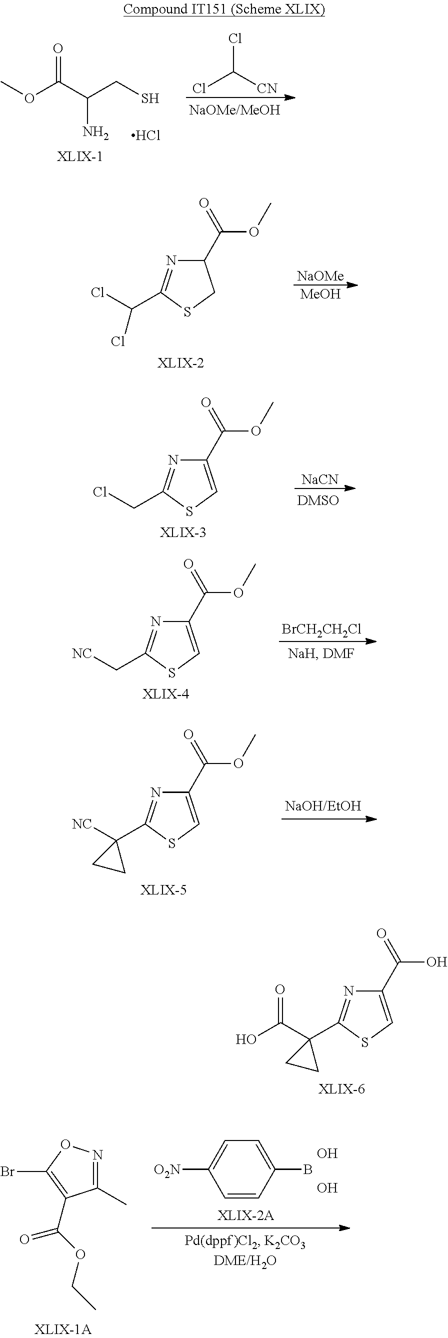

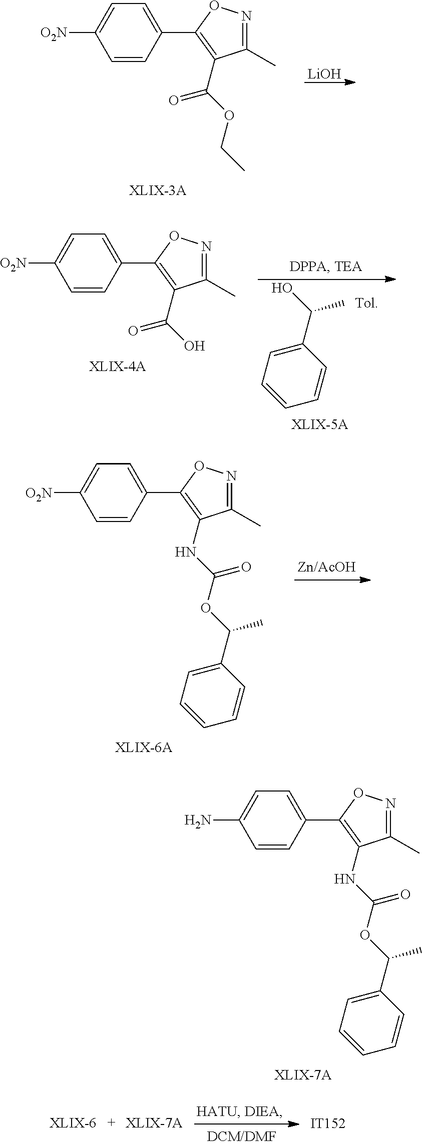

A solution of NaOMe (466 mg, 11.65 mmol) in MeOH (30 mL) was cooled to −10° C. Dichloroacetonitrile (9.35 mL, 116.5 mmol) was added dropwise over 25 min while keeping the internal temperature below 0° C. The mixture was stirred for another 30 min followed by addition a solution of XLIX-1 (20.0 g, 116.5 mmol) in MeOH (30 mL). After addition, the mixture was stirred at rt overnight. The mixture was partitioned between DCM and water. The organic layer was separated and the aqueous layer was then extracted with DCM. The combined organic layers were concentrated to afford crude XLIX-2 (21.3 g, yield: 80%) as a yellow solid.

To a solution of XLIX-2 (21.3 g, 93.5 mmol) in MeOH (20 mL) was added a solution of NaOMe (3.74 g, 93.5 mmol) in MeOH (30 mL) dropwise at rt over 1 h while keeping the temperature below 10° C. After addition, the mixture was then stirred at rt for 16 h. The mixture was partitioned between DCM and water. The organic layer was separated and the aqueous layer was extracted with DCM. The combined organic extracts were concentrated and the residue was purified to afford XLIX-3 (16.0 g, yield: 89%) as a yellowish solid.

To a mixture of NaCN (3.6 g, 50 mmol) in DMSO (17 mL) was added XLIX-3 (8.0 g, 42 mmol) over 20 min at rt and the mixture was stirred at rt overnight. To the mixture was added water. The mixture was extracted with DCM. The combined organic extracts were concentrated and the residue was purified to afford XLIX-4 (600 mg, yield: 8%) as a yellow solid.

To a solution of XLIX-4 (600 mg, 3.3 mmol) in DMF (15 mL) was added NaH (192 mg, 4.8 mmol) portionwise at 0° C. and stirred for 30 min. Then 1-bromo-2-chloroethane (468 mg, 3.3 mmol) was added and the mixture was stirred at rt for 16 h. The mixture was quenched with water and extracted with EtOAc. The combined organic extracts were washed with brine, dried over Na₂SO₄ and filtered. The filtrate was concentrated and the residue was purified to afford XLIX-5 (200 mg, yield: 29%) as a yellow solid.

A mixture of XLIX-5 (200 mg, 0.96 mmol) and aqueous NaOH solution (10 mL, 20%) in EtOH (10 mL) was refluxed for 16 h. After cooling to rt, the mixture was adjusted to pH=4 with 2M HCl. The mixture was extracted with EtOAc. The combined organic extracts were dried over anhydrous Na₂SO₄, filtered and concentrated to give XLIX-6 (200 mg, yield: 97%) as a white solid.

A mixture of XLIX-1A (1.7 g, 7.3 mmol), XLIX-2A (2.0 g, 12 mmol), Pd(dppf)Cl₂ (100 mg, 0.14 mmol) and K₂CO₃ (2.0 g, 14.5 mmol) in DME/H₂O (30 mL/3 mL) was stirred at 90° C. for 12 h under N₂. After cooling to rt, the mixture was diluted with H₂O and extracted with EtOAc. The combined organic extracts were washed with brine, dried over anhydrous Na₂SO₄ and filtered. The filtrate was concentrated and the residue was purified to afford XLIX-3A (1.9 g, yield: 95%) as a yellow solid.

A mixture of XLIX-3A (900 mg, 3.3 mmol) and aqueous LiOH solution (15 mL, 1 mol/L) in THF/H₂O (10 mL/5 mL) was stirred at rt for 16 h. The mixture was adjusted to pH 4 with 2 M HCl and extracted with EtOAc. The combined organic extracts were dried over anhydrous Na₂SO₄ and filtered. The filtrate was concentrated to afford XLIX-4A (700 mg, yield: 86%) as a yellow solid.

A mixture of c XLIX-4A (500 mg, 2 mmol), Et₃N (242.4 mg, 2.4 mmol) and 4A MS (600 mg) in toluene (20 mL) was heated to reflux for 1 h under N₂. Then DPPA (657 mg, 2.4 mmol) and XLIX-5A (293 mg, 2.4 mmol) were added. The mixture was heated at 100° C. under N₂ overnight. After cooling to rt, the reaction mixture was filtered and the filtrate cake was washed with EtOAc. The filtrate was concentrated under reduce pressure and the residue purified to afford XLIX-6A (450 mg, yield: 55%) as a yellow solid.

A mixture of XLIX-6A (1.62 g, 5 mmol) and Zn powder (6.5 g, 100 mmol) in AcOH (50 mL) was stirred at rt for 2 h. The mixture was adjusted to pH=8 with saturated NaHCO₃ solution and extracted with EtOAc. The combined organic extracts were concentrated and the residue was purified to afford XLIX-7A (1.0 g, yield: 67%) as a yellow solid.

A mixture of XLIX-6 (50 mg, 0.23 mmol) and HATU (105 mg, 0.28 mmol) in DMF/DCM (5 mL/10 mL) was stirred at rt for 30 min, then DIEA (148 mg, 1.15 mmol) and XLIX-7A (77 mg, 0.23 mmol) was added. The mixture was stirred at rt for 16 h, quenched with water and extracted with EtOAc. The combined organic extracts were washed with brine, dried over Na₂SO₄ and filtered. The filtrate was concentrated and the residue was purified to give IT152 (50 mg, yield: 40%) as a white solid. ¹HNMR (DMSO-d6, 400 MHz): δ 10.14 (s, 1H), 9.27 (s, 1H), 8.33 (s, 1H), 7.95 (d, J=8.4 Hz, 2H), 7.73 (d, J=8.4 Hz, 2H), 7.42-7.23 (m, 5H), 5.76 (d, J=6.0 Hz, 1H), 2.11 (s, 3H), 1.95 (d, J=4.0 Hz, 2H), 1.82 (d, J=4.0 Hz, 2H), 1.55 (d, J=6.0 Hz, 3H). MS (ESI) m/z (M+H)⁺533.1.

IT193 was prepared by the coupling of 5-(1-(methoxycarbonyl)cyclopropyl)thiophene-3-carboxylic acid with XLIX-7A by (COCl)₂, DMF and pyridine in DCM at 50° C. for 2 h to form the ester intermediated, followed by standard LiOH hydrolysis as a white solid. ¹H NMR (DMSO-d₆, 400 MHz): δ 10.25 (s, 1H), 9.24 (s, 1H), 8.21 (s, 1H), 7.91 (d, J=8.4 Hz, 2H), 7.68 (d, J=8.4 Hz, 2H), 7.41-7.31 (m, 1H), 5.74 (m, 1H), 2.09 (s, 3H), 1.56-1.52 (m, 4H), 1.22 (s, 3H).

IT224 was prepared following the similar procedure for the synthesis of IT152 using 5-(1-carboxycyclopropyl)furan-3-carboxylic acid in place of XLIX-6 as a white solid. ¹HNMR (DMSO-d₆, 400 MHz): δ 10.15 (d, J=6.5 Hz, 1H), 9.23 (s, 1H), 8.30 (d, J=4.3 Hz, 1H), 7.86 (d, J=7.3 Hz, 2H), 7.70 (d, J=7.3 Hz, 2H), 7.46-7.20 (m, 5H), 6.89 (s, 1H), 5.76 (d, J=6.5 Hz, 1H), 2.10 (s, 3H), 1.60-1.46 (m, 4H), 1.34-1.28 (m, 3H). MS (ESI) m/z (M+H)⁺ 516.2.

### Example 44

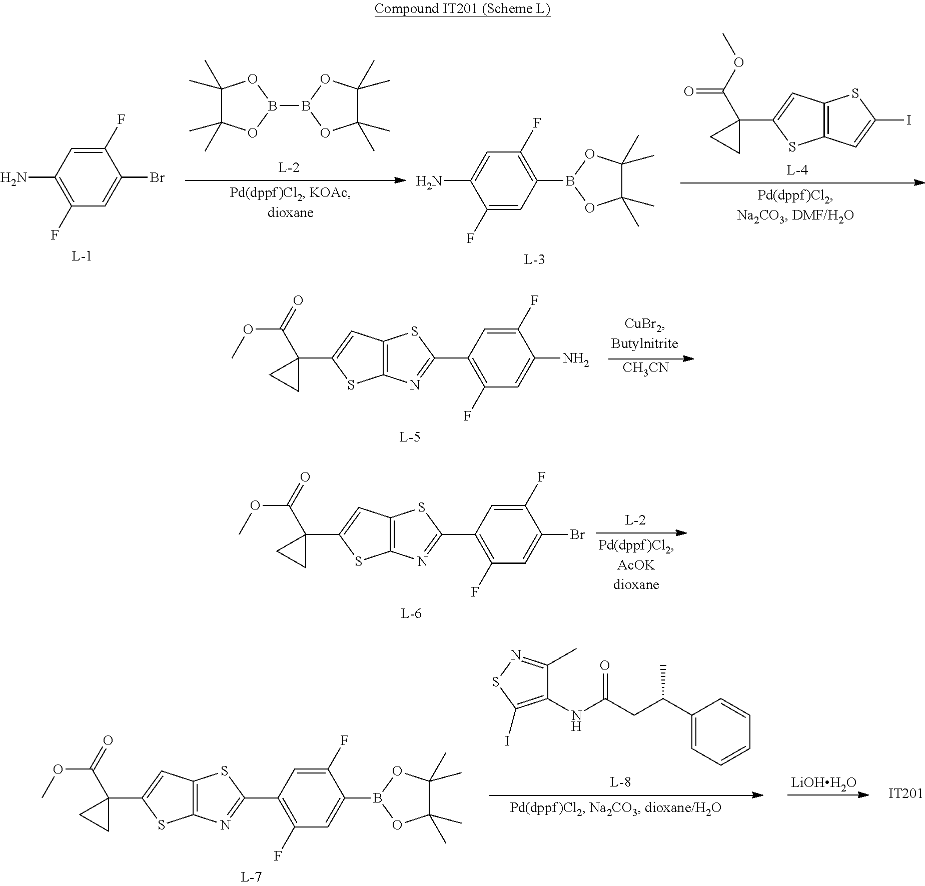

To a stirred mixture of L-1 (4.5 g, 22 mmol), L-2 (6.6 g, 26 mmol) and KOAc (4.3 g, 44 mmol) in dioxane (90 mL) was added Pd(dppf)Cl₂ (1.6 g, 2.2 mmol). The reaction mixture was flushed with N₂ and heated to 80° C. for 3 h. The mixture was diluted with EtOAc, washed with water, dried over Na₂SO₄, filtered and concentrated. The residue was purified to give L-3 (2.5 g, yield: 45%) as a white solid.

The mixture of L-3 (0.87 g, 3.4 mmol), L-4 (1.3 g, 3.4 mmol), Na₂CO₃ (0.72 g, 6.8 mmol) in DME (9 mL) and H₂O (3 mL) was added Pd(PPh₃)₄ (0.4 g, 0.34 mmol). The reaction mixture was flushed with N₂ and heated to 80° C. for 3 h. The mixture was diluted with EtOAc and water, the mixture was extracted with EtOAc. The combined organic layer was dried, concentrated and the residue was purified to give L-5 (0.68 g, yield: 54.8%) as a yellow solid.

The mixture of L-5 (630 mg, 0.7 mmol) and CuBr₂ (460 mg, 1.7 mmol) in CH₃CN (15 mL) was stirred for 15 min at 10° C. Then butyl nitrite (270 mg, 2.6 mmol) was added slowly at 10° C. The reaction mixture was stirred for 1 h at 60° C. The reaction was added water, extracted with EtOAc, the organic layer was washed with 1N HCl and brine, dried over Na₂SO₄, filtered and concentrated. The residue was purified to give L-6 (440 mg, yield: 59.4%) as a yellow solid.

The mixture of L-6 (130 mg, 0.3 mmol), compound 2 (92 mg, 0.36 mmol) were reacted under the same condition as the reaction between L-1 and L-2 to afford L-7 as a yellow solid which was used for next step directly.

The mixture of L-7 (100 mg, 0.27 mmol), L-8 (113 mg, 0.29 mmol), and Na₂CO₃ (56 mg, 0.54 mmol) in dioxane (10 mL) and water (2 mL) was added Pd(dppf)Cl₂ (39 mg, 0.05 mmol). The reaction mixture was flushed with N₂ and stirred at 80° C. for 3 h. The mixture was added water and extracted with EtOAc, the combine organic layers was dried over Na₂SO₄, and concentrated. The residue was purified to give crude intermediate ester (100 mg, yield: 61%) as a yellow solid which was used in the standard LiOH hydrolysis to afford IT201 as the final product. MS (ESI) m/z (M+H)⁺ 598.0. ¹H NMR (400 MHz, DMSO-d₆): δ 9.44 (s, 1H), 8.08-8.12 (m, 1H), 7.61-7.64 (m, 1H), 7.40 (s, 1H), 7.20-7.31 (m, 5H), 5.66 (s, 1H), 2.29 (s, 3H), 1.65-1.67 (m, 2H), 1.43-1.45 (m, 5H).

IT279 was prepared following the similar procedure described in the synthesis of IT201 using (R)-1-phenylethyl (4-iodo-1-methyl-1H-pyrazol-5-yl)carbamate in place of L-8 as a yellow solid. MS (ESI) m/z (M+H)⁺ 581.0. ¹H NMR (400 MHz, Methanol-d₄): δ 7.95-7.99 (m, 1H), 7.77 (s, 1H), 7.26-7.45 (m, 3H), 7.26 (s, 1H), 7.01-7.26 (m, 3H), 5.76 (br, 1H), 3.77 (s, 1H), 1.75-1.77 (m, 2H), 1.50-1.57 (m, 3H), 1.45-1.48 (m, 2H).

### Example 45

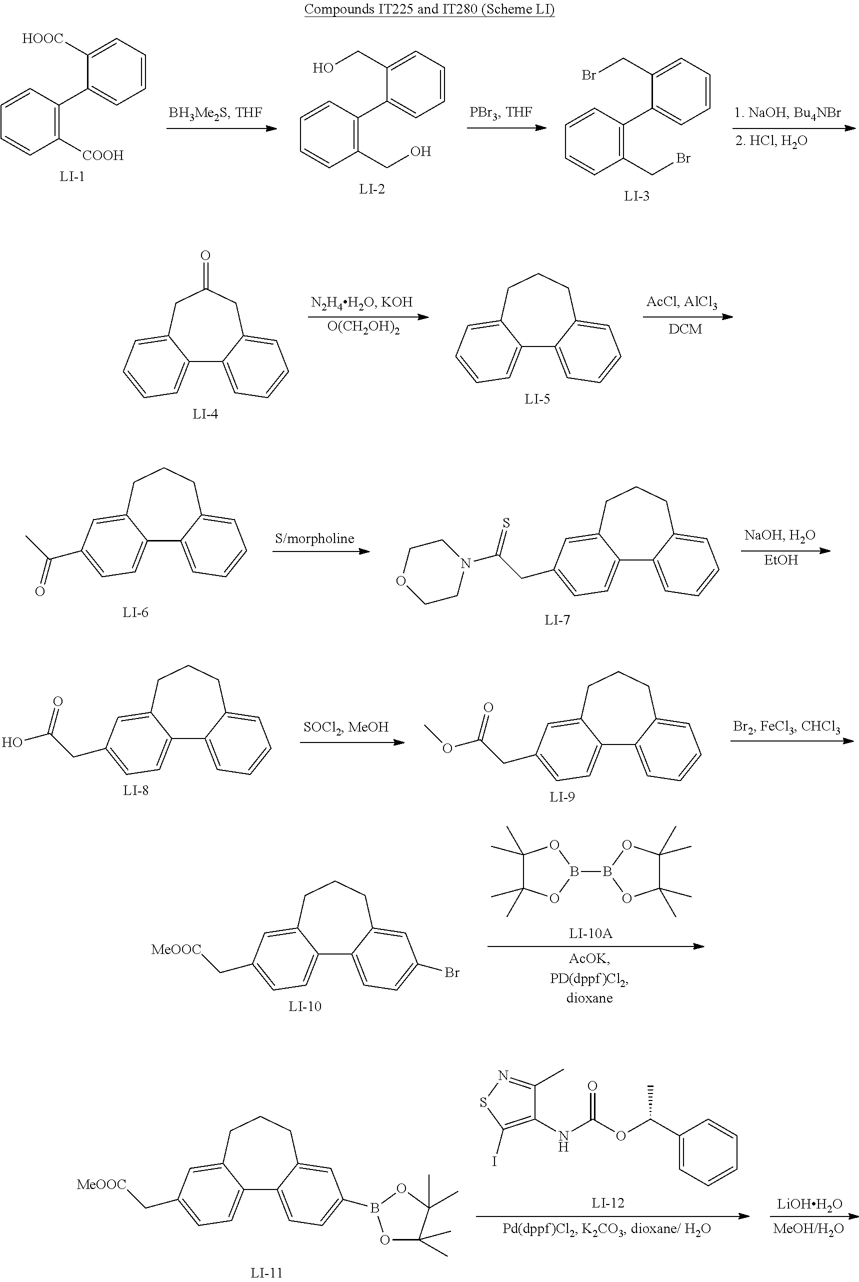

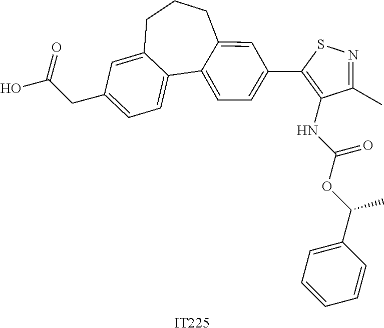 [SMILES: Cc1nsc(-c2ccc3c(c2)CCCc2cc(CC(=O)O)ccc2-3)c1NC(=O)O[C@H](C)c1ccccc1 ; "IT225"]

To a stirred solution of LI-1 (50.0 g, 0.21 mol) in THF (1000 mL) was added dropwise BH₃.Me₂S (62 mL, 0.62 mol) at 0° C. under N₂. The mixture was stirred at 25° C. for 18 h. The mixture was quenched with MeOH. After evaporation, the residue was dissolved with DCM, washed with water, brine, dried over Na₂SO₄, and concentrated to give LI-2 (36.0 g, 80%) as a yellow solid.

To a stirred solution of LI-2 (50.0 g, 0.23 mol) in THF (600 mL) was added dropwise PBr₃ (49 mL, 0.51 mol). The solution was stirred at 25° C. for 18 h. The mixture was quenched with water. DCM was then added to the mixture. After separation, the aqueous layer was extracted with DCM. The combined organic layer was washed with brine, dried over Na₂SO₄, and concentrated. The residue was purified to give LI-3 (100 g, crude) as a white solid.

To a stirred solution of LI-3 (40.0 g, 0.12 mol), TosMic (25.0 g, 0.13 mol) and TBAB (9.3 g, 0.029 mol) in DCM (1200 mL) was added dropwise NaOH (24.5 g, 0.61 mol) in water at 0° C. The mixture was stirred at 25° C. for 18 h. After separation, the aqueous layer was extracted with DCM. Then t-BME (900 mL) and HCl (360 mL, 37%) was added to the combined organic layer. The mixture was stirred for 3 h, and separated. The organic layer was washed with water, sat.aq. NaHCO₃ and brine, dried over Na₂SO₄, and concentrated. The residue was purified to give LI-4 (15.5 g, 62%) as a white solid.

A mixture of LI-4 (15.5 g, 74.5 mmol), N₂H₄.H₂O (12.0 g, 22.35 mmol) and KOH (17.0 g, 303.57 mmol) in 2,2′-oxydiethanol (200 mL) was stirred at 195° C. for 2 h. The mixture was diluted with water and EA. The separated aqueous layer was extracted with EA. The combined organic layer was washed with brine, dried over Na₂SO₄, and concentrated. The residue was purified to give LI-5 (10.0 g, 69%) as a white solid.

To a stirred solution of LI-5 (5.2 g, 26.79 mol) in DCM (50 mL) was added dropwise AcCl (2.1 g, 26.92 mmol). Then AlCl₃ (7.1 g, 53.60 mmol) was added to the mixture in batches. The mixture was stirred at 25° C. for 18 h. The mixture was then poured into ice-water, extracted with DCM. The combined organic layer was washed with brine, dried over Na₂SO₄, and concentrated to give LI-6 (5.0 g, crude) as a yellow oil.

To a stirred solution of LI-6 (5.0 g, 21.19 mol) in morpholine (7.0 g, 80.52 mmol) was added sulfur (1.7 g, 52.97 mmol). Then the mixture was stirred at 150° C. for 2 h. The mixture was cooled to rt, diluted with DCM and water. The separated aqueous layer was extracted with DCM. The combined organic layer was washed with brine, dried over Na₂SO₄, and concentrated to give the LI-7 (10 g, crude) as a yellow oil.

To a stirred solution of LI-7 (10.0 g, 29.67 mmol) in EtOH (30 mL) was added NaOH (8.3 g, 207.70 mmol) in water (15 mL). Then the mixture was stirred at reflux for 18 h. After evaporation, the residue was diluted with DCM and water. After separation, the aqueous layer was washed with PE/EA. Then the aqueous layer was adjusted pH to 2-3 with con. HCl, extracted with DCM. The combined organic layer was washed with brine, dried over Na₂SO₄, and concentrated to give the LI-8 (5 g, crude) as a yellow oil.

To a stirred solution of LI-8 (5.0 g, 19.83 mmol) in MeOH (50 mL) was added SOCl₂ (5 mL). The mixture was stirred at 60° C. for 3 h. After evaporation, the residue was diluted with DCM and water. The separated aqueous layer was extracted with DCM. The combined organic layer was washed with brine, dried over Na₂SO₄, and concentrated. The residue was purified to give LI-9 (3.0 g, 57%) as a white oil.

To a stirred solution of LI-9 (2.0 g, 7.52 mmol), FeCl₃ (247 mg, 1.50 mmol) in CHCl₃ (30 mL) was added Br₂ (1.2 g, 7.52 mmol). The mixture was stirred at 25° C. for 18 h. After evaporation, the residue was diluted with DCM and sat.aq. NH₄Cl. The separated aqueous layer was extracted with DCM. The combined organic layer was washed with brine, dried over Na₂SO₄, and concentrated. The residue was purified by prep-HPLC to give LI-10 (1.3 g, 50%) as a yellow oil.

LI-11 and IT225 were prepared following the similar procedure in the synthesis of L-7 and IT201. IT201: MS (ESI) m/z (M+H)⁺ 513.1. ¹H NMR (Methanol-d₄, 400 MHz): δ 7.42 (s, 1H), 7.37 (m, 3H), 7.31-7.33 (m, 2H), 7.23-7.28 (m, 3H), 7.18 (s, 2H), 5.77-5.78 (m, 1H), 3.63 (s, 2H). 2.41-2.46 (m. 4H). 2.31 (s. 3H). 2.12-2.18 (m. 2H). 1.53-1.55 (d. J=6.4 Hz. 3H).

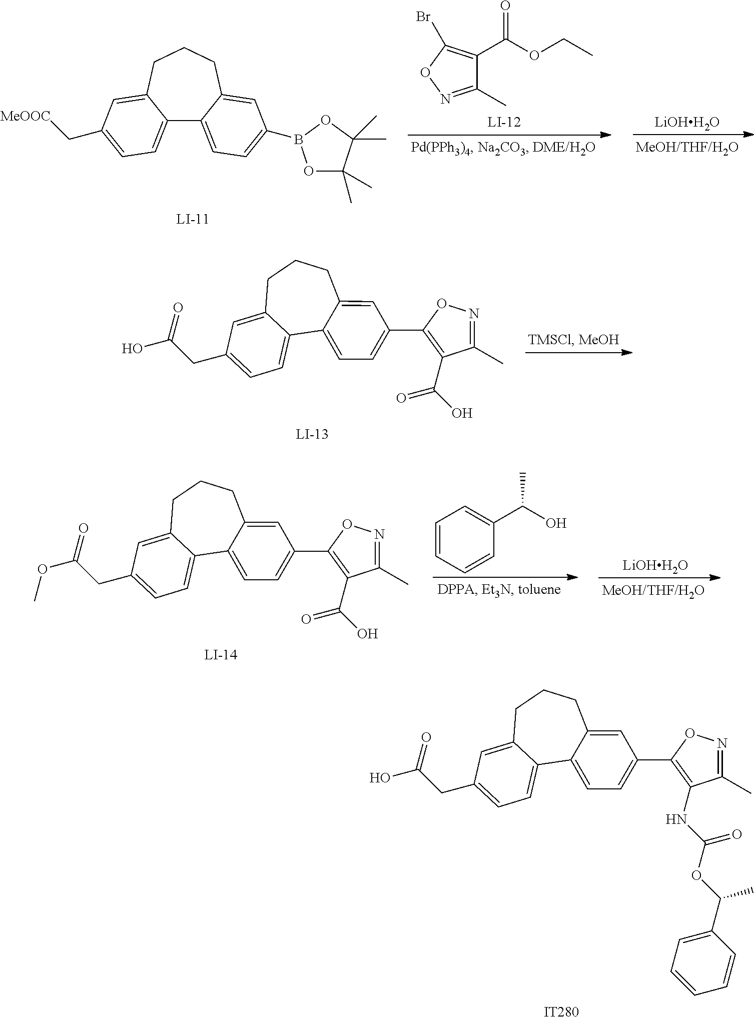

To a solution of LI-11 (1.5 g, 3.82 mmol), LI-7 (890.0 mg, 3.82 mmol) and Na₂CO₃ (810.0 mg, 7.64 mmol) in DME/H₂O (15 mL/5 mL) was added Pd(PPh₃)₄ (441.0 mg, 0.382 mmol) under N₂. The mixture was stirred at 80° C. for 4 h. After being cooled to rt, the mixture was diluted with EA and water. The organic layer was washed with brine, dried over Na₂SO₄, and concentrated. The residue was purified to give the methyl ester intermediate which was subject to standard LiOH hydrolysis to afford LI-13 (800.0 mg, yield 81%).

To the solution of LI-13 (800.0 mg, 2.12 mmol) in absolute MeOH was added TMSCl (0.5 mL) at 0° C., the mixture was stirred for 1 h. The reaction mixture was quenched by water, and then concentrated. The aqueous layer was extracted with EA. The combined organic layers were washed with brine, dried over Na₂SO₄, concentrated to afford LI-14 (870.0 mg, crude) as a white solid.

To the solution of LI-14 (300 mg, 0.77 mmol) in toluene was added TEA (156 mg, 1.54 mmol), (S)-1-phenylethanol (103 mg, 0.84 mol) and DPPA (254 mg, 0.92 mmol). The mixture was stirred at reflux for 2 h. After being cooled to rt, the mixture was diluted with EA and water. The combined organic layers were washed with brine, dried over Na₂SO₄, concentrated. The residue was subject to standard LiOH hydrolysis to afford IT280 as a white solid (34.5 mg, yield 24%). ¹H NMR (Methanol-d₄, 400 MHz): δ 7.71 (d, J=8.0 Hz, 1H), 7.64 (s, 1H), 7.43 (m, 3H), 7.33-7.35 (m, 3H), 7.29 (d, J=7.6 Hz, 2H), 7.21 (s, 1H), 5.82-5.83 (m, 1H), 3.65 (s, 2H), 2.47 (m, 4H), 2.16-2.19 (m, 5H), 1.60 (d, J=6.4 Hz, 3H).

### Example 46

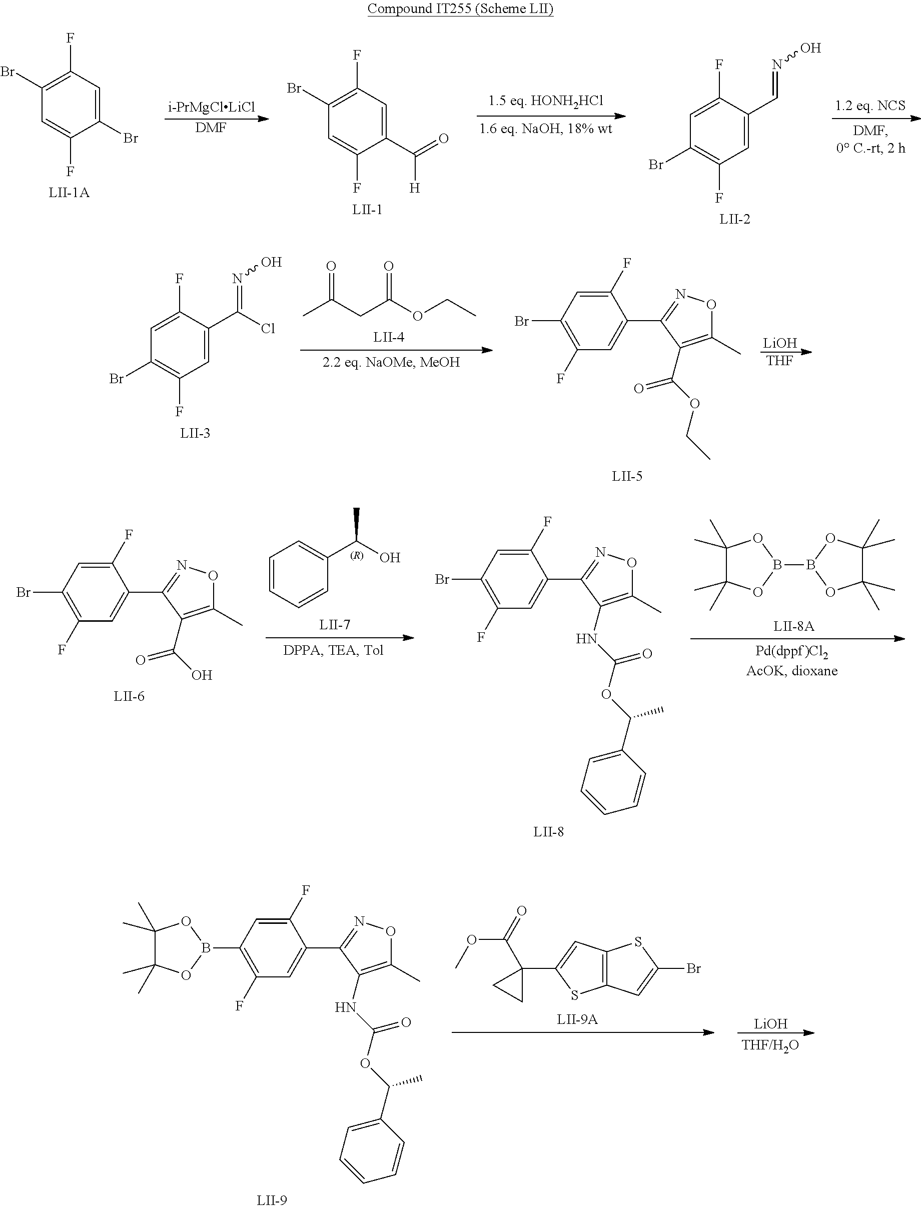

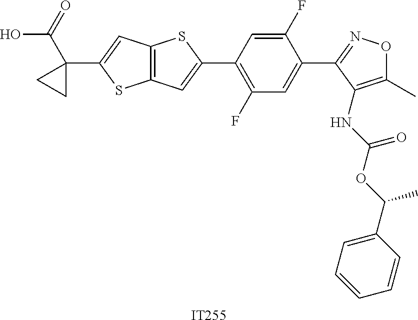 [SMILES: Cc1onc(-c2cc(F)c(-c3cc4sc(C5(C(=O)O)CC5)cc4s3)cc2F)c1NC(=O)O[C@H](C)c1ccccc1 ; "IT255"]

To a solution of LII-1A (36 g, 132 mmol) in THF (450 mL) was added i-PrMgCl.LiCl (132 mmol) dropwise at −60° C. The mixture was stirred for 1 h. Then, DMF (200 mL) was added and the reaction mixture was stirred overnight. NH₄Cl (500 mL) was added, extracted with EtOAc. The combined organic layers were dried over anhydrous Na₂SO₄. The reaction solution was concentrated under reduced pressure to afford LII-1 (29.4 g, crude).

To a solution of LII-1 (19.0 g, 86 mmol), HONH₂HCl (6.56 g, 95 mmol) in dry EtOH (8 mL) and H₂O (30 mL) was added ice (40 g). Then, a solution of NaOH (4.6 g) dissolved in H₂O (10 mL) was added dropwise to the suspension. The reaction mixture was stirred at rt for 2 h. Then reaction mixture was acidized by 4N HCl. The precipitate was then filtered, washed with water and dried under vacuum, to give LII-2 (19.0 g, yield: 93.6%).

NCS (8.46 g, 63.6 mmol) was added to stirred solution of LII-2 (12.0 g, 51.0 mmol) in a mixture of DMF (60 mL) at 0° C. The mixture was stirred at 25° C. for 16 h. The mixture was poured into ice water and extracted with EtOAc. The combined organic layers were washed with brine, dried over Na₂SO₄, concentrated to give LII-3 (13.72 g, crude).

A solution of compound NaOMe (3.6 g, 66.3 mmol) in MeOH (500 mL) was added a solution of LII-4 (6.64 g, 51 mmol) in MeOH (200 mL). The reaction mixture was stirred at rt for 30 min. Then, a solution of LII-3 (13.7 g, 51 mmol) in MeOH (200 mL) was added slowly, and stirring was continued for another 24 hrs. The reaction mixture was concentrated and purified to give LII-5 (6.3 g, yield: 35.7%).

The mixture of LII-5 (2.50 g, 7.48 mmol) in THF (16 mL), H₂O (4 mL) was added LiOH (358 mg, 15.0 mmol). The reaction mixture was stirred for 16 hrs at 25° C. The reaction mixture was diluted with H₂O and extracted with EtOAc, the water layer was acidified by 3N HCl to pH=4, extracted with EtOAc, the organic layer was washed with brine, dried over Na₂SO₄, concentrated to afford LII-6 (700 mg, yield 80.2%).

The mixture of LII-6 (3.6 g, 10.7 mmol), LII-7 (1.57 g, 12.9 mmol), DPPA (3.55 g, 12.9 mmol), Et₃N (2.17 g, 21.5 mmol) and 4A MS (1.8 g) in toluene (80 mL) was stirred for 2 hrs at 80° C. After concentrated, the residue was partitioned between H₂O and DCM, the aqueous phase was extracted with DCM, and the combined organic layer was washed with brine, dried over Na₂SO₄, concentrated. The residue was purified to afford LII-8 (4.52 g, yield: 91.9%).

The coupling between LII-8 and bis(pinacolato)diboron, the Suzuki-Coupling of LII-9 and LII-9A and subsequent LiOH hydrolysis were followed the similar procedure described in the synthesis of IT155. IT255: ¹H NMR (400 MHz, DMSO-d₆): δ 9.10 (s, 1H), 7.85˜7.89 (m, 1H), 7.51˜7.53 (m, 1H), 7.28˜7.34 (m, 5H), 5.62˜5.63 (m, 1H), 2.35 (s, 1H), 1.44˜1.45 (t, 1H). MS (ESI) m/z (M+H)⁺ 580.1.

### Example 47

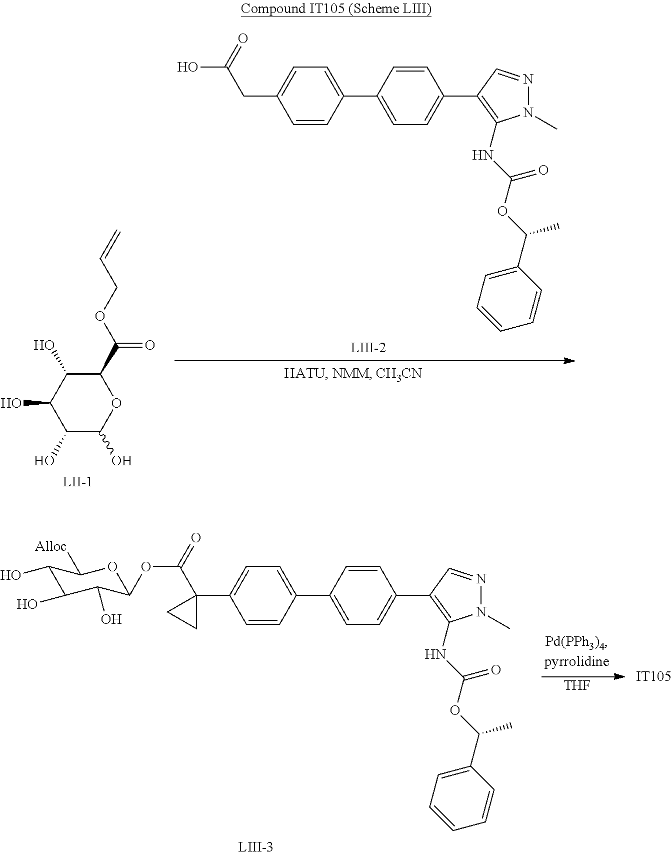

The preparation of LIII-1 and LIII-2 has been disclosed in the synthesis of Compound 238 and Compound 171 of U.S. Pub. No. 2013/0072449.

A mixture of LIII-1 (360 mg, 0.75 mmol), LIII-2 (175 mg, 0.75 mmol) and HATU (284 mg, 0.75 mmol) were stirred in 4 mL of dry acetonitrile with N-methylmorpholine (151 mg, 1.50 mmol) was heated to 60° C. for 24 h. The mixture was diluted with H₂O and extracted with EA. The combined organic layer was washed with brine, dried and concentrated. The residue was purified by prep-TLC to afford LIII-3 (80 mg, yield 15%).

Pd(PPh₃)₄ (13 mg, 0.011 mmol) was added to LIII-3 (80 mg, 0.11 mmol) and pyrrolidine (8 uL, 0.11 mmol) with stirring in THF (1 mL) at 0° C. for 10 min. Evaporation of solvent left a yellow gum, which was purified by HPLC to afford IT105 (30 mg, yield 40%). ¹H NMR (400 MHz, Methanol-d₄): δ 7.87 (s, 1H), 7.58-7.61 (m, 5H), 7.40-7.52 (m, 8H), 5.840 (br, 1H), 5.51 (d, J=8.4 Hz, 1H), 3.95 (d, J=9.6 Hz, 1H), 3.76 (s, 3H), 3.50-3.55 (m, 1H), 3.41-3.46 (m, 1H), 3.27-3.31 (m, 1H), 1.75-1.82 (m, 2H), 1.63 (br, 3H), 1.33-1.35 (m, 2H). MS (ESI) m/z (M+H)⁺ 658.0.

### Example 48

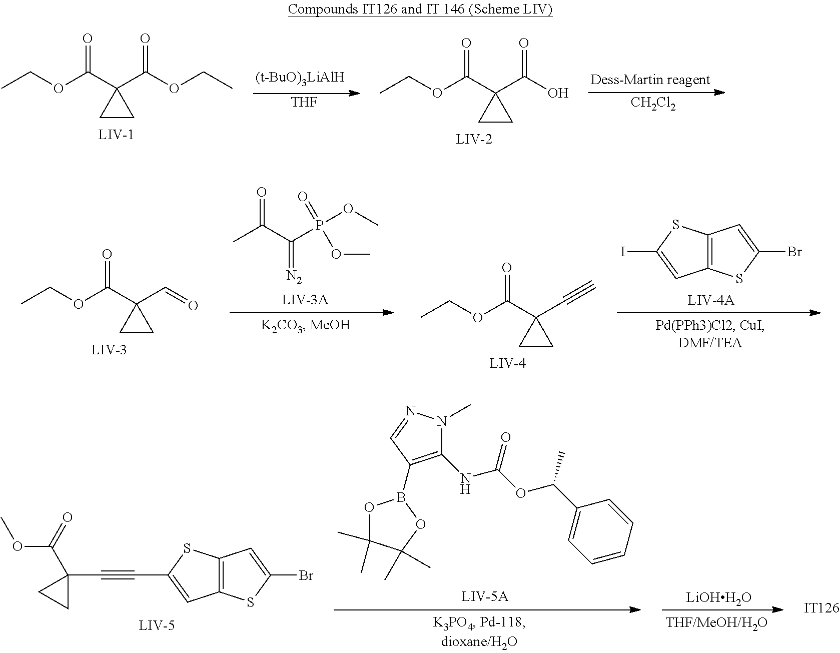

A suspension of lithium tri-tert-butoxyaluminum hydride (30 g, 117.98 mmol) in 120 mL of anhydrous THF was added to a solution of LIV-1 (10 mL, 53.74 mmol) in anhydrous THF (190 mL) slowly. After addition, the mixture was heated to 60° C. and stirred overnight. The mixture was quenched by addition of 2N HCl. Then the mixture was extracted with EA. The combined organic layer was washed with brine, dried and concentrated. The residue was purified to afford LIV-2 (6.2 g, yield 81%).

To a solution of LIV-2 (6.2 g, 43 mmol) in 200 mL of dichloromethane was added Dess-Martin periodinane (22.6 g, 60.2 mmol) in portions at 0° C. The mixture was stirred for 4 h at 0° C. The reaction mixture was quenched by the addition of Na₂S₂O₃ aqueous solution. Then NaHCO₃ aqueous solution was added to pH=7. The mixture was extracted with DCM. The combined organic layer was washed with brine, dried and concentrated. The resulting slurry was treated with hexane, filtered and the filtrate was concentrated to afford LIV-3 (5.1 g, yield 84%).

To a stirred mixture of LIV-3 (124 mg, 0.87 mmol) and dimethyl (1-diazo-2-oxopropyl) phosphonate (200 mg, 1.04 mmol) in 2 mL of anhydrous methanol was added K₂CO₃ (240 mg, 1.74 mmol). The mixture was stirred for 5 h at rt. The mixture was poured into 5 mL of ice-water, extracted with hexane. The combined organic layer was washed with brine, dried and concentrated to 3 mL to afford a solution of LIV-4 in hexane, which was used for next step directly.

Argon gas was bubbled through a mixture of LIV-4A (300 mg, 0.87 mmol) in 3 mL of DMF/TEA (v/v=3/1). Pd(PPh₃)Cl₂ (60 mg, 0.087 mmol) and CuI (50 mg, 0.26 mmol) was added. Then a solution of LIV-4 (0.87 mmol) in 3 mL of hexane was added. The mixture was stirred overnight at rt. The mixture was diluted with H₂O, extracted with EA. The combined organic layer was washed with brine, dried and concentrated. The residue was purified by prep-TLC to afford LIV-5 (60 mg, yield 20%).

LIV-5 was reacted with LIV-5A following the same Pd-118 catalyzed coupling condition as described in the preparation of XI-2 in Example 41, followed by standard LiOH hydrolysis to afford the final product IT126 (20 mg, yield 45%). ¹H NMR (400 MHz, Methanol-d₄): δ 7.74 (s, 1H), 7.31-7.45 (m, 5H), 7.02-7.24 (m, 2H), 5.83 (q, J=6.4 Hz, 1H), 3.69 (s, 3H), 1.58-1.65 (m, 5H), 1.41-1.44 (m, 2H). MS (ESI) m/z (M+H)⁺ 492.0.

IT121 was prepared by the Suzuki Coupling of methyl 1-(5-(4-bromo-2,5-difluorophenyl)thieno[3,2-b]thiophen-2-yl)cyclopropanecarboxylate and LIV-5A following the similar procedure described in the synthesis of IT126. ¹H NMR (400 MHz, DMSO-d₆): δ 9.74 (s, 1H), 8.08 (s, 1H), 7.70-7.74 (m, 2H), 7.30-7.43 (m, 7H), 5.75 (s, 1H), 3.68 (s, 3H), 1.65-1.68 (m, 2H), 1.54 (s, 3H), 1.40-1.43 (m, 2H). MS (ESI) m/z (M+H)⁺ 580.0.

IT122 was similarly prepared using the corresponding isothiazolyl carbamate. ¹H NMR (400 MHz, DMSO-d₆): δ 9.45 (s, 1H), 7.97 (s, 1H), 7.87-7.91 (m, 1H), 7.54-7.58 (m, 1H), 7.26-7.39 (m, 6H), 5.70-5.71 (d, J=6.4 Hz, 1H), 2.31 (s, 3H), 1.64-1.66 (m, 2H), 1.50-1.51 (d, J=6 Hz, 3H), 1.41-1.44 (m, 2H), MS (ESI) m/z (M+H)⁺ 597.0.

IT282 was prepared by the Suzuki-Coupling of methyl 1-(5-(2-chloro-5-fluoro-4-(4,4,5,5-tetramethyl-1,3,2-dioxaborolan-2-yl)phenyl)thieno[3,2-b]thiophen-2-yl)cyclopropanecarboxylate and LIV-5A following the similar procedure described in the synthesis of IT126. ¹H NMR (400 MHz, DMSO-d₆): δ 12.75 (s, 1H), 9.75 (s, 1H), 7.81 (s, 1H), 7.75-7.76 (m, 1H), 7.65-7.67 (d, J=7.2 Hz, 1H), 7.57-7.60 (m, 1H), 7.30-7.39 (m, 6H), 5.76 (s, 1H), 3.68 (s, 3H), 1.64-1.67 (m, 2H), 1.55 (br, 3H), 1.40-1.43 (m, 2H). MS (ESI) m/z (M+H)⁺ 596.0.

IT281 was similarly prepared using the corresponding isothiazolyl carbamate. ¹H NMR (400 MHz, DMSO-d₆): δ 12.77 (s, 1H), 9.46 (s, 1H), 7.92 (s, 1H), 7.74-7.79 (m, 2H), 7.27-7.79 (m, 6H), 5.71-5.73 (d, J=5.6 Hz, 1H), 2.32 (s, 3H), 1.67-1.68 (m, 2H), 1.51 (s, 3H) 1.43 (m, 2H). MS (ESI) m/z (M+H)⁺ 613.0.

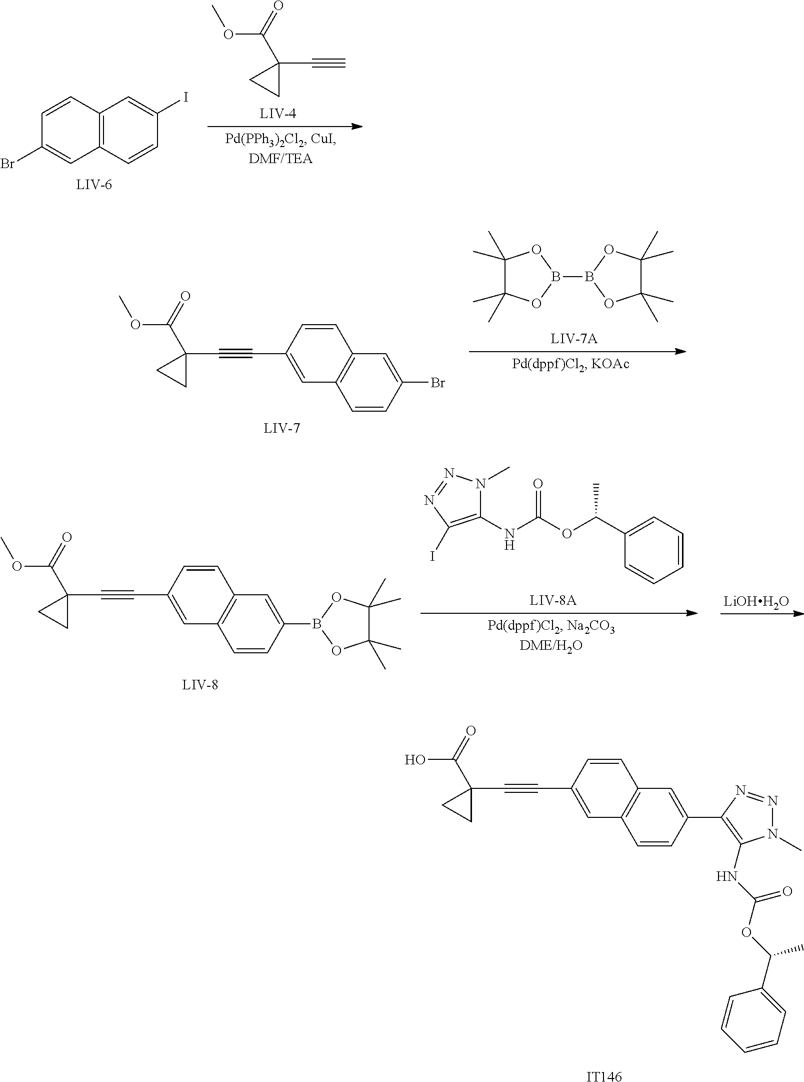

To a stirred mixture of LIV-4, LIV-6 (350 mg, 1.05 mmol) and CuI (60 mg, 0.316 mmol) in DMF/TEA=3/1 (12 mL) was added Pd(PPh₃)₂Cl₂ (73.9 mg, 0.105 mmol). The reaction mixture was flushed with N₂ and stirred at rt for 2 h. The reaction mixture was poured into water, and the product was extracted with EA. The EA extract was washed with water and brine and dried over anhydrous Na₂SO₄. After evaporation of the solvent, the residue was purified to give LIV-7 (80 mg, yield 23.3%).

The mixture of LIV-7 (40 mg, 0.122 mmol), LIV-7A (62 mg, 0.244 mmol), KOAc (24 mg, 0.244 mmol) and Pd(dppf)Cl₂ (8.9 mg, 0.012 mmol) in 3 mL of dioxane was heated to reflux under argon for 4 h. The mixture was concentrated, the residue was partitioned between H₂O and DCM, the aqueous phase was extracted with DCM, and the combined organic layer was washed with brine, dried over Na₂SO₄, concentrated. The residue was purified to afford LIV-8 (40 mg, yield 86.9%).

IT146 was prepared by reacting LIV-8 and LIV-8A following the similar procedure in the synthesis of III-5, followed by standard LiOH hydrolysis. ¹H NMR (Methanol-d₄, 400 MHz): δ 8.13 (s, 1H), 7.97 (s, 1H), 7.87 (s, 2H), 7.69 (br, 1H), 6.97-7.69 (m, 6H), 5.89 (br, 1H), 3.96 (s, 3H), 1.65-1.68 (m, 3H), 1.46-1.49 (m, 2H), 1.35 (br, 2H). MS (ESI) m/z (M+H)+481.1.

### Example 49

### Exemplary Compounds of Formula (II) with Amido L⁵ Linker

### Synthesis of IT371

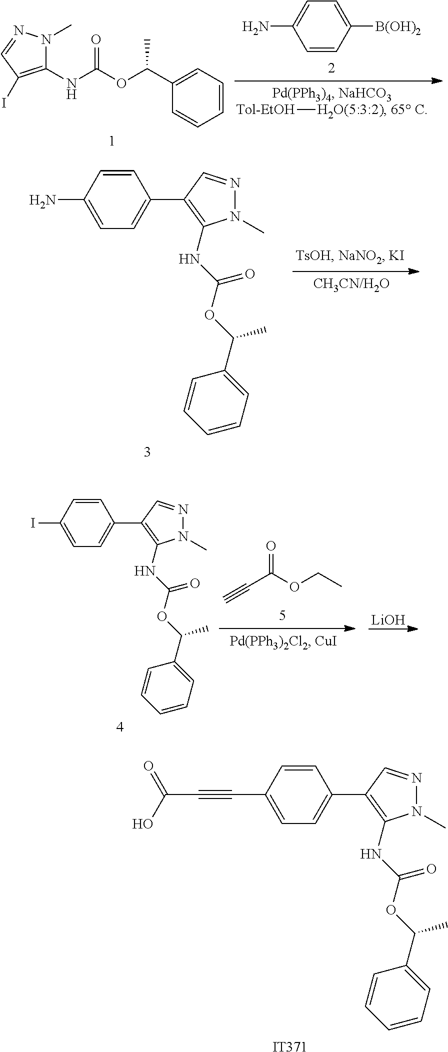

To a solution of compound 1 (50 g, 135 mmol, 1 eq.) in toluene/ethanol/water (v/v/v=3/1, 1000 mL) were added sodium bicarbonate (68 g, 810 mmol, 6 eq.) and compound 2 (27.6 g, 202 mmol, 1.5 eq.). The resulting mixture was purged with nitrogen gas, then Pd(PPh₃)₄ (4 g, 3.375 mmol, 0.025 eq.) was added. The mixture was heated to 65° C. for 16 hrs under N₂, then the mixture was poured into water. The product was extracted with EtOAc (300 mL×3). The combined organic layer was dried over sodium sulfate, filtered and concentrated in vacuo. The residue was purified by chromatography on silica gel (PE:EtOAc=3:1) to afford compound 3 (15 g, yield 33%).

To a solution of p-toluenesulfonic acid (18.4 g, 107 mmol, 3 eq.) in acetonitrile (26 mL) was added compound 3 (12 g, 35.7 mmol, 1 eq.) in acetonitrile (52 mL) dropwise. Then the stirred mixture was cooled to 10-15° C. KI (14.8 g, 89 mmol, 2.5 eq) and NaNO₂ (4.92 g, 71.4 mmol, 2 eq.) in water was added to the reacting mixture. The mixture was stirred at rt for 3 hrs. Then the mixture was poured into water and extracted with EtOAc. The combined organic layer was washed with sodium sulfite solution and brine, dried over sodium sulfate, filtered and concentrated in vacuo. The residue was purified to afford compound 4 (11.5 g, yield 72%).

To a stirred solution of compound 4 (white solid, 1 g, 2.23 mmol) in THF (60 ml) were added Pd(PPh₃)₂Cl₂ (180 mg, 0.223 mmol), copper(I) iodide (20 mg, 0.0669 mmol) and potassium carbonate (617 mg, 4.46 mmol). The reaction mixture was flushed with nitrogen and stirred at rt for 30 mins. Then compound 5 (876 mg, 8.92 mmol) was added. The mixture was stirred at refluxing for 24 hrs. The mixture was diluted with EtOAc, the combined organic layer was washed with water and brine, dried over sodium sulfate, filtered and concentrated. The residue was purified to afford the ester intermediate which was subject to LiOH hydrolysis to afford IT371 as a brown solid. ¹HNMR (d-DMSO, 400 HMz) δ 9.69 (s, 1H), 7.88 (s, 1H), 6.80-7.68 (m, 10H), 5.76 (s, 1H), 3.64 (s, 3H), 1.22-1.67 (m, 3H). MS (ESI) m/z (M+H)⁺ 390.0.

### Synthesis of IT398

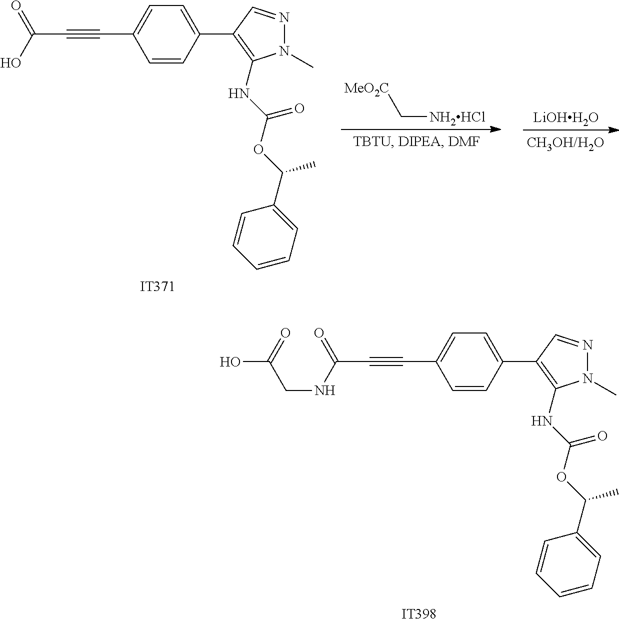

To a stirred mixture of IT371 (30 mg, 0.08 mmol), methyl 2-aminoacetate hydrochloride (10.6 mg, 0.096 mmol), TBTU (33.8 mg, 0.104 mmol) in DMF (2.5 mL) was added DIPEA (41 mg, 0.32 mmol). The reaction mixture was stirred for 2 hrs. The mixture was diluted with EtOAc, washed with water and brine, dried over Na₂SO₄, filtered and concentrated. The residue was purified to afford the intermediate ester, which was subjected to standard LiOH hydrolysis to arrive at the final product IT398 (25 mg, yield: 86%). ¹H NMR (400 MHz, DMSO-d₆): δ 9.70 (s, 1H), 8.93 (s, 1H), 7.86 (s, 1H), 7.54 (s, 4H), 7.23-7.42 (m, 5H), 5.76 (s, 1H), 3.77-3.78 (d, J=5.6 Hz, 2H), 3.63 (s, 3H), 1.55-1.56 (d, J=5.2 Hz, 3H), MS (ESI) m/z (M+H)⁺ 447.2.

IT399 was prepared following the similar procedure described in the synthesis of IT398 replacing methyl 2-aminoacetate hydrochloride with (R)-methyl 2-aminopropanoate hydrochloride. ¹H NMR (400 MHz, DMSO-d₆): δ 9.68 (s, 1H), 9.10 (d, J=7.2 Hz, 1H), 7.87 (s, 1H), 7.54 (br, 5H), 7.34-7.42 (m, 4H), 5.76 (brs, 1H), 4.23-4.30 (m, 1H), 3.63 (s, 3H), 1.55 (brs, 3H), 1.32 (d, J=7.6 Hz, 3H), MS (ESI) m/z (M+H)⁺ 461.2.

IT400 was prepared following the similar procedure described in the synthesis of IT398 replacing methyl 2-aminoacetate hydrochloride with (S)-methyl 2-aminopropanoate hydrochloride. ¹H NMR (400 MHz, DMSO-d₆): δ 9.66 (s, 1H), 9.08-9.09 (d, J=7.2 Hz, 1H), 7.84 (s, 1H), 7.22-7.51 (m, 9H), 5.73 (s, 1H), 4.22-4.28 (m, 1H), 3.60 (s, 3H), 1.52-1.53 (d, J=5.2 Hz, 3H), 1.28-1.30 (d, J=7.6 Hz, 3H). MS (ESI) m/z (M+H)⁺ 461.2.

IT401 was prepared following the similar procedure described in the synthesis of IT398 replacing methyl 2-aminoacetate hydrochloride with (R)-methyl 2-amino-2-phenylacetate hydrochloride. ¹H NMR (400 MHz, DMSO-d₆): δ 9.66 (s, 1H), 9.57-9.59 (d, J=7.6 Hz, 1H), 7.86 (s, 1H), 7.55 (br, 4H), 7.38-7.45 (m, 10H), 5.76 (brs, 1H), 5.44 (d, J=7.2 Hz, 1H), 3.63 (s, 3H), 1.54 (s, 3H), MS (ESI) m/z (M+H)⁺ 523.2.

IT402 was prepared following the similar procedure described in the synthesis of IT398 replacing methyl 2-aminoacetate hydrochloride with (S)-methyl 2-amino-2-phenylacetate hydrochloride. ¹H NMR (400 MHz, DMSO-d₆): δ 9.64 (s, 1H), 9.52-9.54 (d, J=7.6 Hz, 1H), 7.83 (s, 1H), 7.18-7.51 (m, 14H), 5.72 (brs, 1H), 5.39-5.41 (d, J=7.2 Hz, 1H), 3.59 (s, 3H), 1.52 (s, 3H), MS (ESI) m/z (M+H)⁺ 523.2.

IT403 was prepared following the similar procedure described in the synthesis of IT398 replacing methyl 2-aminoacetate hydrochloride with (R)-methyl 2-amino-3-methylbutanoate hydrochloride. ¹H NMR (400 MHz, DMSO-d₆): δ 9.68 (s, 1H), 9.00 (d, J=8.0 Hz, 1H), 7.87 (s, 1H), 7.56-7.65 (m, 5H), 7.34-7.42 (m, 4H), 5.76 (br, 1H), 4.17-4.21 (m, 1H), 3.63 (s, 3H), 2.07-2.16 (m, 1H), 1.55 (br, 3H), 0.92-0.95 (m, 6H). MS (ESI) m/z (M+H)⁺ 489.3.

IT404 was prepared following the similar procedure described in the synthesis of IT398 replacing methyl 2-aminoacetate hydrochloride with (S)-methyl 2-amino-3-methylbutanoate hydrochloride. ¹H NMR (400 MHz, DMSO-d₆): δ 9.66 (s, 1H), 8.98 (d, J=8.0 Hz, 1H), 7.84 (s, 1H), 7.31-7.53 (m, 9H), 5.73 (br, 1H), 4.14-4.18 (m, 1H), 3.60 (s, 3H), 2.04-2.10 (m, 1H), 1.53 (br, 3H), 0.89-0.97 (m, 6H). MS (ESI) m/z (M+H)⁺ 489.2.

IT405 was prepared following the similar procedure described in the synthesis of IT398 replacing methyl 2-aminoacetate hydrochloride with methyl 1-aminocyclopropanecarboxylate hydrochloride. ¹H NMR (400 MHz, DMSO-d₆): δ 9.66 (s, 1H), 9.24 (s, 1H), 7.83 (s, 1H), 7.32-7.50 (m, 9H), 5.73 (s, 1H), 3.60 (s, 3H), 1.53 (s, 3H), 1.33-1.36 (m, 2H), 1.01-1.04 (m, 2H). MS (ESI) m/z (M+H)⁺ 473.2.

IT429 was prepared following the similar procedure described in the synthesis of IT398 replacing methyl 2-aminoacetate hydrochloride with 3-aminophenyl propionate. ¹H NMR (400 MHz, DMSO-d₆): δ 10.99 (s, 1H), 9.65 (s, 1H), 8.27 (s, 1H), 7.82-7.85 (m, 2H), 7.65 (d, J=7.6 Hz, 1H), 7.57 (s, 5H), 7.39-7.47 (m, 5H), 5.73 (s, 1H), 3.60 (s, 3H), 1.52 (s, 3H). MS (ESI) m/z (M+H)⁺ 509.2.

IT432 was prepared following the similar procedure described in the synthesis of IT398 replacing methyl 2-aminoacetate hydrochloride with ethyl 3-aminopropanoate hydrochloride. ¹H NMR (400 MHz, DMSO-d₆): δ 9.35 (s, 1H), 8.56 (s, 1H), 7.81 (s, 1H), 7.49-7.56 (m, 4H), 7.31-7.40 (m, 5H), 5.75-7-5.79 (m, 1H), 3.63-3.65 (d, 3H), 3.37-3.38 (d, 2H), 2.45-2.47 (m, 2H), 1.49 (s, 3H), MS (ESI) m/z (M+H)⁺ 461.3.

IT466 was prepared following the similar procedure described in the synthesis of IT398 replacing methyl 2-aminoacetate hydrochloride with ethyl 3-(methylamino)propanoate. ¹H NMR (400 MHz, Methanol-d₄): δ 7.77 (s, 1H), 7.33-7.49 (m, 9H), 5.79 (br, 1H), 4.00-4.04 (m, 1H), 3.70-3.73 (m, 1H), 3.69 (s, 3H), 3.35, 3.03 (s, s 3H), 2.69-2.73 (m, 1H), 2.60-2.64 (m, 1H), 1.59-1.60 (d, J=4.0 Hz, 3H). MS (ESI) m/z (M+H)⁺ 475.3.

### Synthesis of IT430

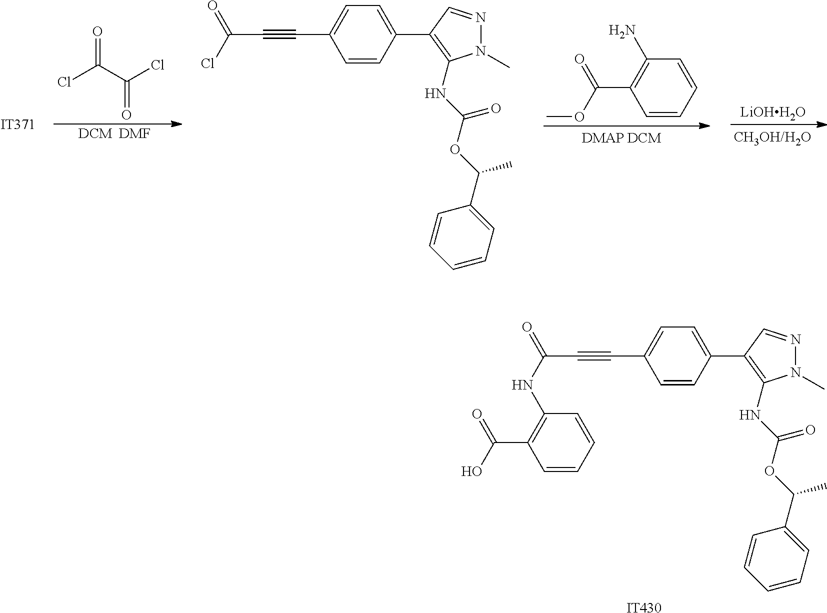

To a stirred mixture of IT371 (100 mg, 0.257 mmol), oxalyl chloride (71 mg, 0.514 mmol), in DCM (2.5 mL) was added DMF (0.1 mL). The reaction mixture was stirred for 2 hrs. The mixture was concentrated and the crude product was used for next step directly by adding methyl 2-aminobenzoate (95 mg, 0.51 mmol) and DMAP (3 mg, 0.0257 mmol). The reaction mixture was stirred for 2 hrs. The mixture was diluted with EtOAc, washed with water and brine, dried and purified to give the ester intermediate (70 mg, yield: 54%) which was subject to standard LiOH hydrolysis to afford IT430 (30 mg, yield: 45%). ¹H NMR (400 MHz, DMSO-d₆): δ 11.68 (s, 1H), 9.69 (s, 1H), 8.36-8.38 (d, J=6.8 Hz, 1H), 8.01-8.03 (d, J=8.0 Hz, 1H), 7.89 (s, 1H), 7.59-7.67 (m, 6H), 7.36-7.42 (m, 4H), 7.24-7.28 (m, 1H), 5.77 (s, 1H), 3.64 (s, 3H), 1.56 (br, 3H). MS (ESI) m/z (M+H)⁺ 509.3.

IT431 was prepared following the similar procedure described in the synthesis of IT430 replacing methyl 2-aminobenzoate with methyl 4-aminobenzoate. ¹H NMR (400 MHz, DMSO-d₆): δ 11.11 (s, 1H), 9.66 (s, 1H), 7.90 (d, J=8.4 Hz, 2H), 7.85 (s, 1H), 7.73 (d, J=8.8 Hz, 2H), 7.57 (br, 5H), 7.31-7.40 (m, 4H), 5.73 (s, 1H), 3.60 (s, 3H), 1.53 (m, 3H). MS (ESI) m/z (M+H)⁺ 509.2.

### Example 50

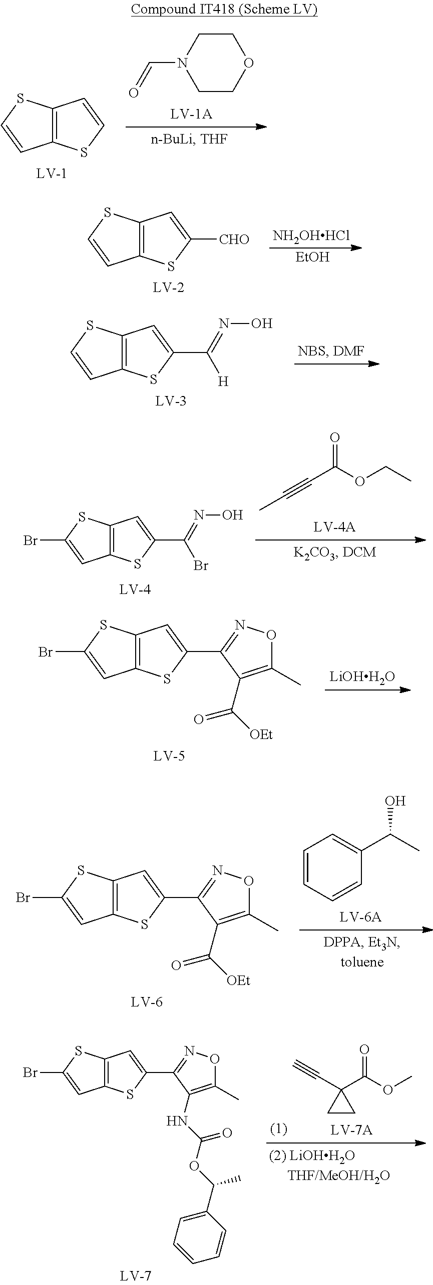

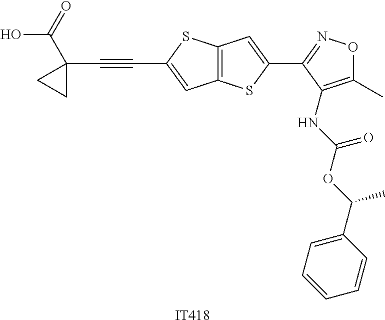 [SMILES: Cc1onc(-c2cc3sc(C#CC4(C(=O)O)CC4)cc3s2)c1NC(=O)O[C@H](C)c1ccccc1 ; "IT418"]

To a stirred solution of LV-1 (500 mg, 3.6 mmol) in THF (20 mL) was added n-BuLi (1.44 mL, 3.6 mmol) at −78° C. under N₂. The mixture was stirred at this temperature for 1 h, LV-1A (414 mg, 3.6 mmol) was added dropwise. Then it was stirred at rt for 4 hrs. It was poured to a saturated solution of NH₄Cl and was extracted with EA. The combined organic layers were washed with brine, and concentrated. The residue was purified to give LV-2 (200 mg, yield: 33%).

To a stirred solution LV-2 (0.9 g, 5.33 mmol) in 40 mL of EtOH was added NH₂OH HCl (0.37 g, 5.33 mmol) and KOAc (1 g, 10.7 mmol). The solution was stirred at rt for 12 hrs. The solution was diluted with DCM, washed with water. The organic layer was dried over Na₂SO₄, concentrated to provide LV-3 (0.9 g, yield: 92%) which was used in the next step directly.

To a stirred solution of LV-3 (500 mg, 2.73 mmol) in 20 mL of DMF was added NBS (973 mg, 5.5 mmol). The solution was stirred at rt for 1 h. The mixture was diluted with EA and washed with water. The organic layer was dried over Na₂SO₄, concentrated to provide LV-4 (700 mg, yield: 76%) which was used in the next step directly.

To a stirred solution of LV-4 (1 g, 2.9 mmol) in 20 mL of DCM was added LV-4A (0.33 g, 2.9 mmol) and K₂CO₃ (0.8 g, 5.8 mmol). The solution was heated to 40° C. under nitrogen for 12 hrs. The mixture was filtered, the filtrate was concentrated and purified to provide LV-5 (0.63 g, yield: 58%), which was subjected to LiOH (355 mg, 8.5 mmol) deprotection to afford LV-6 (500 mg, yield: 87%).

A mixture of LV-6 (1.6 g, 4.65 mmol), LV-6A (0.68 g, 5.58 mmol), DPPA (1.53 g, 5.58 mmol) and TEA (0.94 g, 9.3 mmol) in toluene (30 mL) was stirred at 90° C. for 12 hrs. The toluene was removed and diluted with EA, washed with water. The organic layer was dried over Na₂SO₄, concentrated and purified to afford LV-7 (1 g, yield: 48%).

LV-7A was prepared following the same procedure for the synthesis of LIV-4 in Exmaple 48. LV-8 was prepared following the similar procedure described in the synthesis of LIV-7, which was subject to LiOH hydrolysis to afford IT418. ¹H NMR (400 MHz, Methanol-d₄): δ 7.38-7.51 (m, 6H), 7.04-7.09 (m, 1H), 5.82 (d, J=6.4 Hz, 1H), 2.36 (s, 3H), 1.62-1.68 (m, 5H), 1.46-1.48 (m, 2H). MS (ESI) m/z (M+H)⁺ 493.1.

### Example 51

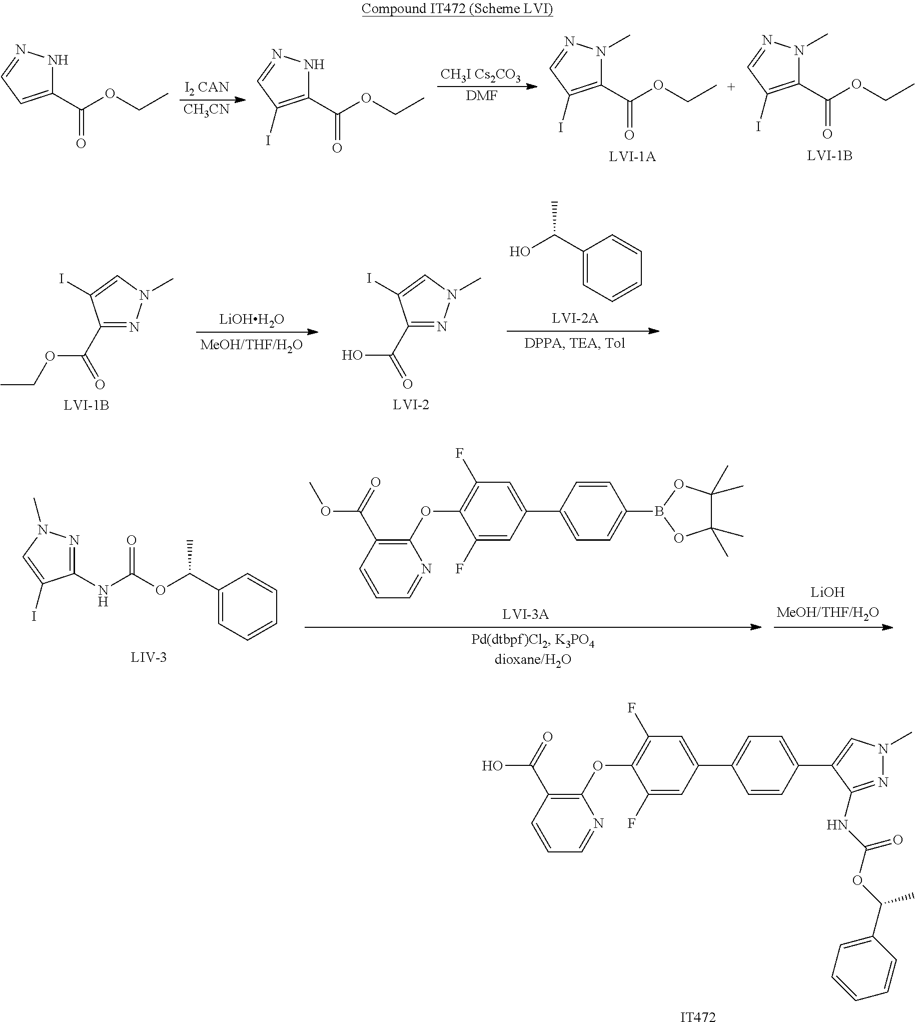

The preparation of LVI-1A, LVI-1B through LVI-3 were disclosed Exmample 6-A (synthesis of IT014).

The mixture of LVI-3 (400 mg, 1.07 mmol), LVI-3A (622 mg, 1.3 mmol), K₃PO₄ (459 mg, 2.16 mmol) and Pd(dtbpf)Cl₂ (35 mg, 0.054 mmol) in dioxane/H₂O (10 mL, v/v=5/1) was MW at 80° C. under nitrogen for 15 min. After concentrated, the residue was partitioned between H₂O and EtOAc, the aqueous phase was extracted with EtOAc, and the combined organic layer was washed with brine, dried over Na₂SO₄, concentrated. The residue was purified to afford the intermediate ester (350 mg, yield: 54.34%), which was subject to LiOH hydrolysis to afford the final product IT472 (90 mg, yield: 26.9%). Sodium salt IT472a: ¹H NMR (DMSO-d₆, 400 MHz): δ 9.20 (s, 1H), 8.07 (s, 1H), 7.86-7.91 (m, 2H), 7.70-7.72 (d, J=8.0 Hz, 2H), 7.55-7.59 (m, 4H), 7.04-7.07 (m, 6H), 5.70 (q, J=5.6 Hz, 1H), 3.81 (s, 3H), 1.44-1.46 (br, 3H). MS (ESI) m/z (M+H)⁺ 571.3.

IT473 was prepared following the similar procedure described in the synthesis of IT472 using (R)-1-phenylethyl (4-(2,5-difluoro-4-iodophenyl)-1-methyl-1H-pyrazol-5-yl)carbamate in place of LVI-3 as a white solid. Sodium salt IT473a: ¹H NMR (DMSO-d₆, 400 MHz): δ 9.76 (br, 1H), 7.87-7.89 (m, 1H), 7.84 (d, J=7.2 Hz, 1H), 7.73 (d, J=2.4 Hz, 1H), 7.59-7.64 (m, 1H), 7.47 (d, J=8.4 Hz, 2H), 7.27-7.43 (m, 5H), 7.03-7.05 (m, 1H), 5.73 (br, 1H), 3.65 (s, 3H), 1.50 (br, 3H). MS (ESI) m/z (M+H)⁺ 607.2.

The foregoing syntheses are exemplary and can be used as a starting point to prepare a large number of additional compounds. Additional compounds of Formula (I) through Formula (XVI) can be prepared according to those synthetic schemes described herein. Those skilled in the art will be able to recognize modifications of the disclosed syntheses and to devise routes based on the disclosures herein; all such modifications and alternate routes are within the scope of the claims.

### Example 52

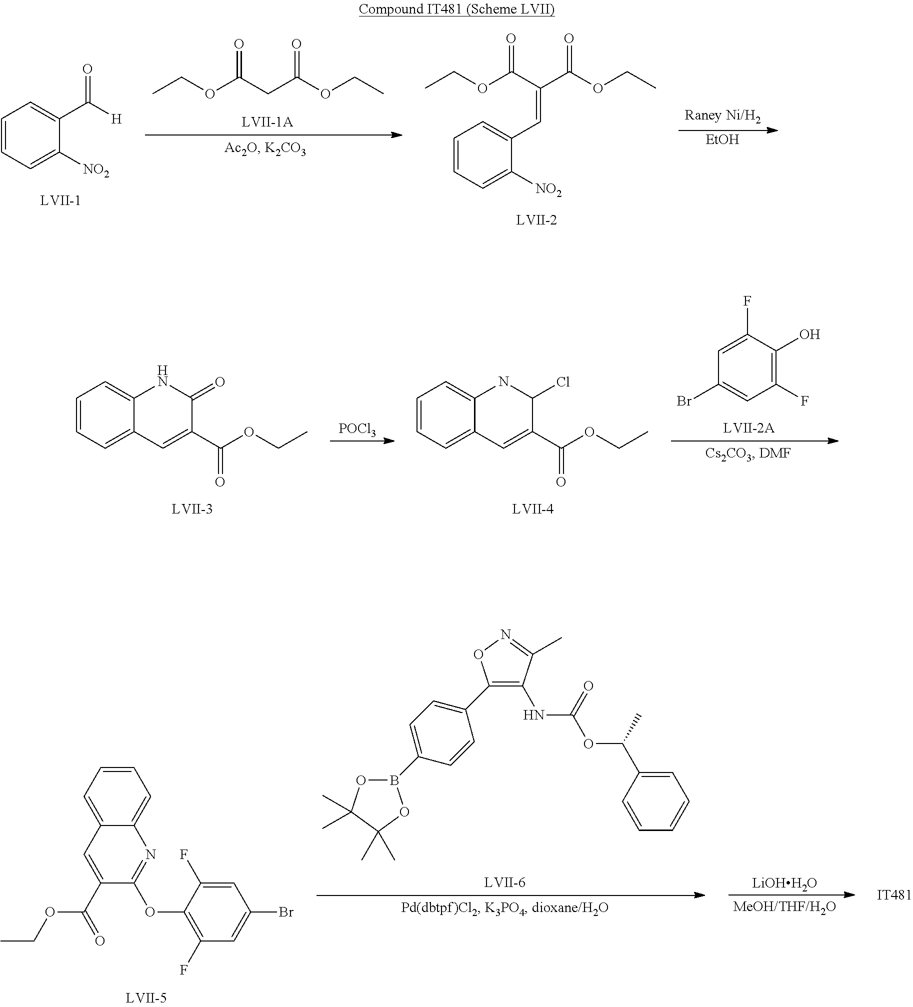

K₂CO₃ (13.72 g, 99.26 mmol) was added to a solution of LVII-1 (10 g, 66.17 mmol), LVII-1A (10.6 g, 66.17 mmol) in Ac₂O (20 ml) was stirred at 80° C. under N₂ overnight. The mixture was then washed with water, extracted with EA, dried over MgSO₄, filtered, concentrated. The residue was triturated with EtOH at 0° C. to afford LVII-2 (17 g, yield 88%) as a yellow solid.

A solution of LVII-2 (5 g, 17.6 mmol) in EtOH (20 ml) was added Raney Ni (6.4 g, 108 mmol) under H₂ at rt overnight. The mixture was then filtered, washed with MeOH, dried over MgSO₄, and filtered. The combined organic layer was then concentrated to afford LVII-3 (3 g, yield 81% as a yellow solid).

A solution of LVII-3 (2 g, 9.2 mmol) in POCl₃ (30 ml) was stirred at 110° C. overnight. Then, the mixture was concentrated, washed with water, extracted with EA, dried over MgSO₄, and filtered. The combined organic layer was then concentrated to give a residue which was purified to afford LVII-4 (0.9 g, yield 42%) as a yellow solid.

A solution of LVII-4 (0.5 g, 2.12 mmol), LVII-2A (0.89 g, 4.24 mmol), Cs₂CO₃ (2.07 g, 6.36 mmol) in DMF (15 ml) was stirred at 110° C. overnight. Then the mixture was washed with water, extracted with EA, dried over MgSO₄, and filtered. The combined organic layer was then concentrated and purified to afford LVII-5 (0.5 g, yield 58%).

The Suzuki-coupling of LVII-5 and LVII-6 and the subsequent LiOH hydrolysis were conducted following the similar procedure described in Example 51 to afford IT481. ¹H NMR (400 MHz, DMSO-d₆): δ 9.39 (s, 1H), 9.04 (s, 1H), 8.15 (d, J=8.4 Hz, 1H), 7.99 (d, J=7.6 Hz, 2H), 7.87-7.89 (m, 2H), 7.79-7.82 (m, 3H), 7.78-7.80 (m, 2H), 7.11-7.66 (m, 5H), 5.76-5.78 (d, J=6.4 Hz, 1H), 2.14 (s, 3H), 1.56 (d, J=8.4 Hz, 3H), MS (ESI) m/z (M+H)⁺ 622.3.

IT482 was prepared following the similar procedure described in the synthesis of IT481 using 4-bromophenol in place of LVII-2A. ¹H NMR (400 MHz, DMSO-d₆): δ 9.33 (s, 1H), 8.92 (s, 1H), 8.09 (d, J=8.0 Hz, 1H), 7.72-7.82 (m, 7H), 7.64 (d, J=8.0 Hz, 1H), 7.54-7.56 (m, 1H), 7.02-7.42 (m, 7H), 5.74-5.76 (m, 1H), 2.12 (s, 3H), 1.54 (d, J=6.0 Hz, 3H). MS (ESI) m/z (M+H)⁺ 586.4.

### Example 53

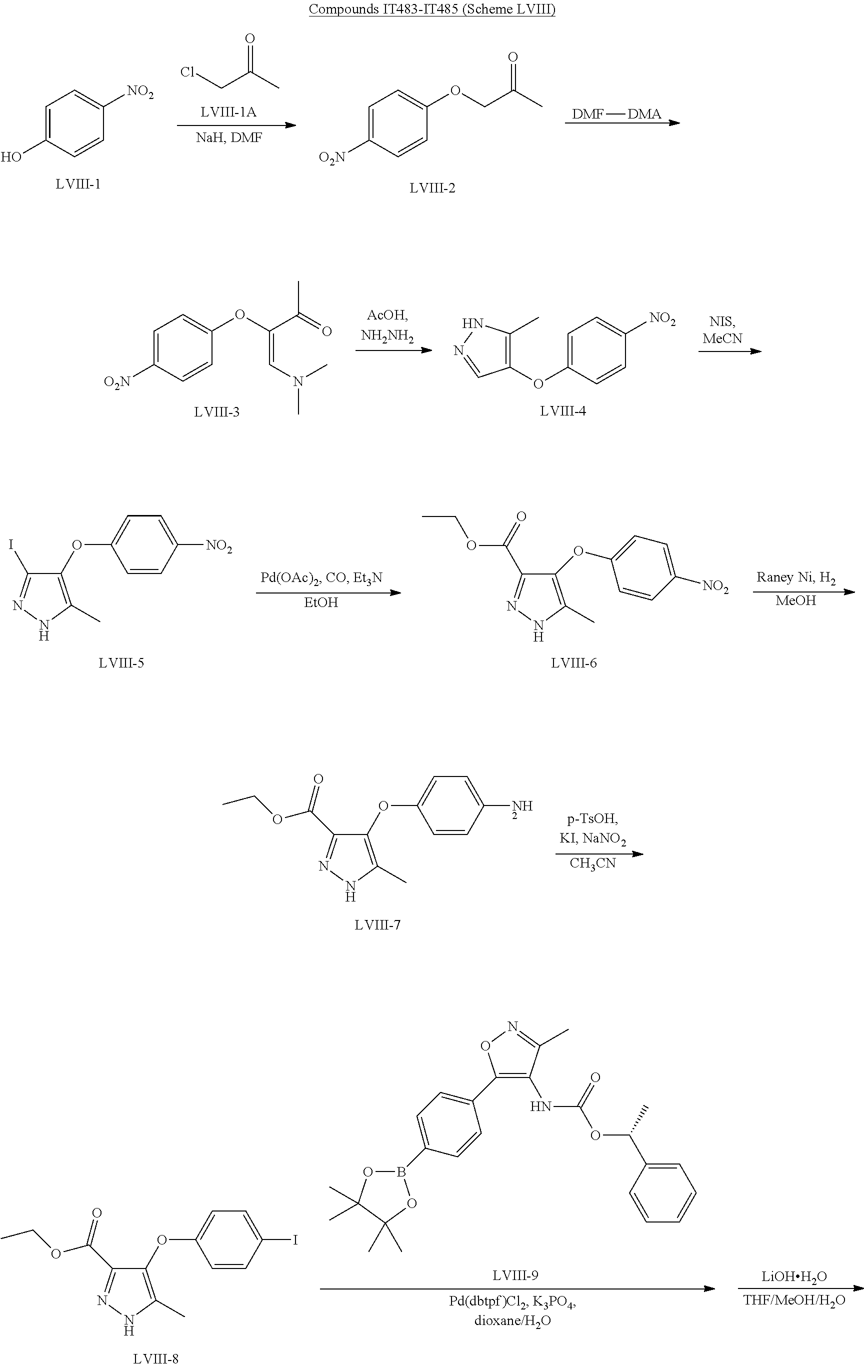

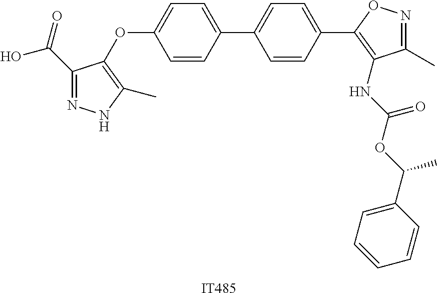 [SMILES: Cc1noc(-c2ccc(-c3ccc(Oc4c(C(=O)O)n[nH]c4C)cc3)cc2)c1NC(=O)O[C@H](C)c1ccccc1 ; "IT485"]

To a solution of LVIII-1 (20 g, 143.8 mmol) in DMF (200 mL) was added slowly NaH (11.5 g, 287.6 mmol) and LVIII-1A (25.2 g, 272.4 mmol) at 0° C. The mixture was stirred at rt overnight. The reaction was quenched with H₂O and was diluted with EtOAc. The organic layer was washed with NaOH (0.05 mol/L), dried over Na₂SO₄, filtered and concentrated to afford LVIII-2 (22 g, yield 78.3%).

A solution of LVIII-2 (5.0 g, 25.6 mmol) in DMF-DMA (6.1 g, 51.2 mmol) was stirred at 100° C. under N₂ for 1 h. After concentrated, the mixture was diluted with EtOAc, washed with brine, dried over Na₂SO₄, filtered and concentrated. The residue was purified to give LVIII-3 (1.9 g, yield 29.6%).

To a stirred solution of LVIII-3 (5.8 g, 23.2 mmol) in EtOH (60 mL) was added AcOH (4.6 g, 76.6 mmol) and NH₂NH₂.H₂O (6.96 g, 145 mmol). The mixture was stirred at 75° C. for 2 h. After standard work-up procedure described above, the residue was purified to give LVIII-4 (2.8 g, yield 55%).

To a stirred solution of LVIII-4 (2.8 g, 12.7 mmol) in CH₃CN (50 mL) was added NIS (5.5 g, 31.9 mmol). The mixture was stirred at 75° C. overnight. After concentrated, the residue was subject to standard work-up procedure described above and purified to give LVIII-5 (3.2 g, yield 72.4%).

To a stirred solution of LVIII-5 (300 mg, 0.87 mmol) in EtOH (5 mL) saturated with CO was added Pd(OAc)₂ (39 mg, 0.17 mmol) and Et₃N (263 mg, 2.61 mmol). The mixture was stirred at 100° C. under 100 psi for 24 h. After concentrated, the mixture was diluted with EtOAc, subject to standard work-up procedure, and purified to give LVIII-6 (160 mg, yield 63%).

To a stirred solution of LVIII-6 (100 mg, 0.34 mmol) in MeOH (5 mL) was added Raney Ni (199 mg, 3.44 mmol). The suspension was degassed under vacuum and purged with H₂ several times. The mixture was stirred under H₂ balloon at rt for 12 h. The mixture was filtered and concentrated under vacuum to afford LVIII-7 (70 mg, yield 78%).

To a solution of p-TsOH.H₂O (3.16 g, 18.36 mmol) in MeCN (20 mL) was added LVIII-7 (1.6 g, 6.1 mmol). The resulting suspension of amine salt was cooled to 10-15° C. and to this added, gradually, a solution of NaNO₂ (845 mg, 12.3 mmol) and KI (2.54 g, 15.3 mmol) in H₂O (10 mL). The reaction mixture was stirred for 10 min, then allowed to warm to 20 C and stirred for 1 h. To the mixture was then added H₂O, NaHCO₃, Na₂S₂O₃, extracted with EA, dried over MgSO₄, and filtered. The combined organic layer then concentrated and purified by a silica gel to afford LVIII-8 (1.6 g, yield 70%).

The Suzuki-coupling of LVIII-8 and LVII-9 and the subsequent LiOH hydrolysis were conducted following the similar procedure described in Example 51 to afford IT485. ¹H NMR (400 MHz, DMSO-d₆): δ 9.28 (s, 1H), 8.86 (s, 1H), 7.69-7.76 (m, 4H), 7.64 (d, J=8.8 Hz, 2H), 7.30-7.40 (m, 4H), 7.19-7.23 (m, 1H), 6.92 (d, J=8.8 Hz, 2H), 5.73 (d, J=6.0 Hz, 1H), 2.09 (s, 3H), 2.04 (s, 3H), 1.56 (d, J=6.0 Hz, 3H). MS (ESI) m/z (M+H)⁺ 539.3.

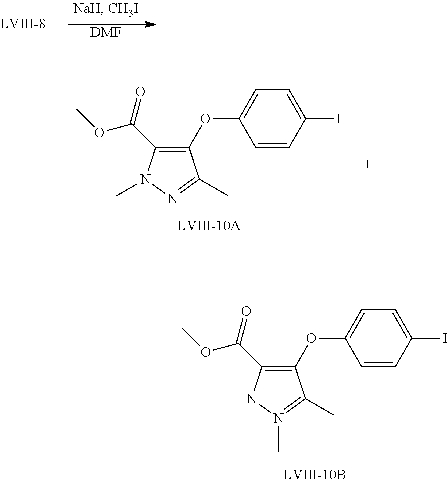

To the solution of LVIII-8 (300 mg, 0.81 mmol) in DMF (6 mL) was added NaH (58 mg, 2.4 mmol) at 0° C. After stirring 0.5 h, CH₃I (914 mg, 6.4 mmol) was added. The mixture was stirred at rt overnight. The mixture was filtered, washed with water, extracted with EA, dried over MgSO₄, and filtered. The combined organic layer was then concentrated and purified to afford LVIII-10A (130 mg, yield 45%) and LVIII-10B (140 mg, yield 47%).

IT483 was prepared by Suzuki-coupling of LVIII-10A and LVIII-9 followed by LiOH hydrolysis following the similar procedure described in Example 51. ¹H NMR (400 MHz, DMSO-d₆): δ 9.31 (s, 1H), 7.73-7.80 (m, 4H), 7.68 (d, J=8.8 Hz, 2H), 7.34-7.43 (m, 4H), 7.09-7.22 (m, 1H), 6.98 (d, J=8.8 Hz, 2H), 5.76 (d, J=6.4 Hz, 1H) 4.04 (s, 3H), 2.12 (s, 3H), 2.01 (s, 3H), 1.55 (d, J=6.0 Hz, 3H). MS (ESI) m/z (M+H)⁺ 553.4.

IT484 was prepared by Suzuki-coupling of LVIII-10B and LVIII-9 followed by LiOH hydrolysis following the similar procedure described in Example 51. ¹H NMR (400 MHz, DMSO-d₆): δ 9.31 (s, 1H), 7.73-7.80 (m, 4H), 7.66 (d, J=8.8 Hz, 2H), 7.34-7.43 (m, 4H), 7.23-7.24 (m, 1H), 6.95 (d, J=8.8 Hz, 2H), 5.76 (d, J=6.8 Hz, 1H), 3.85 (s, 3H), 2.13 (s, 6H), 1.55 (d, J=6.0 Hz, 3H). MS (ESI) m/z (M+H)⁺ 553.4.

### Example 54

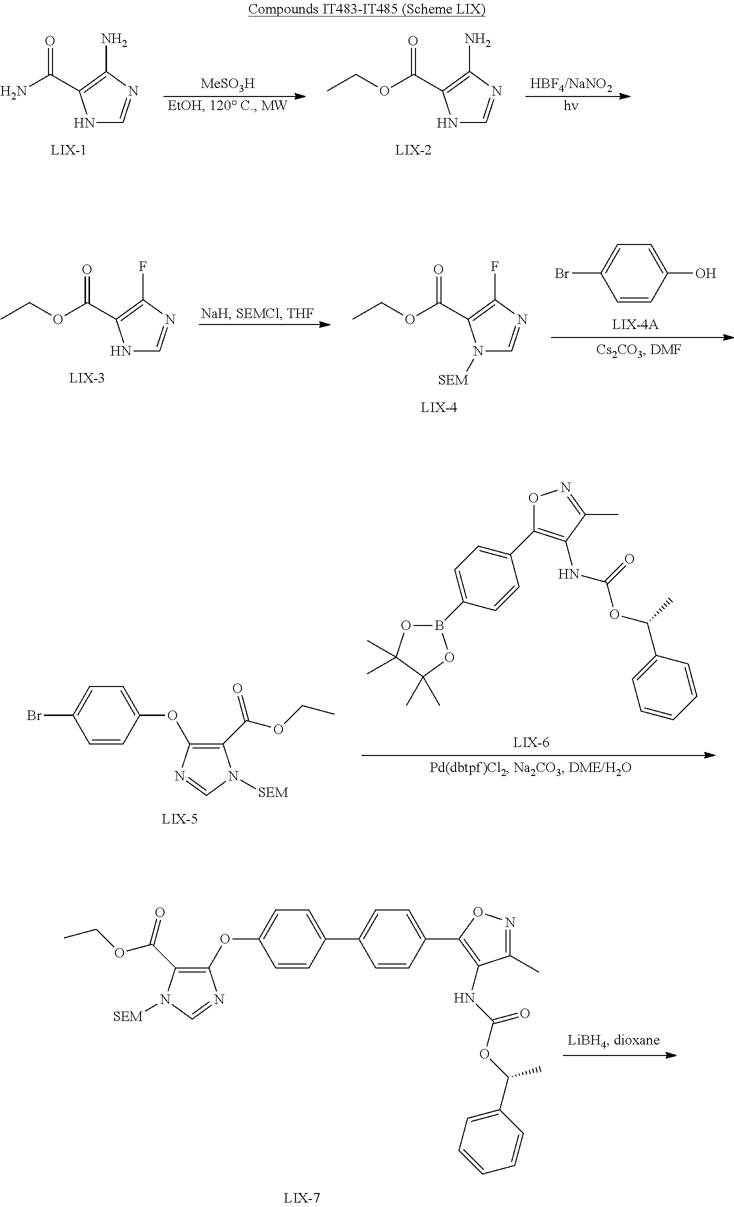

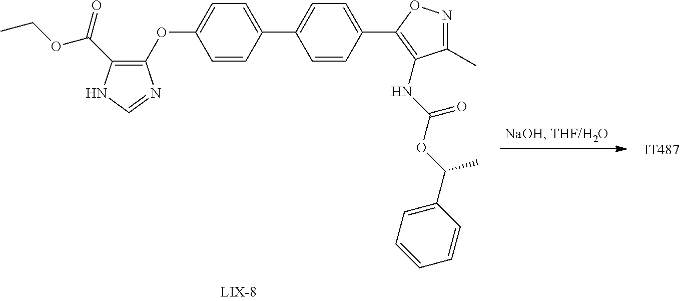 [SMILES: CCOC(=O)c1[nH]cnc1Oc1ccc(-c2ccc(-c3onc(C)c3NC(=O)O[C@H](C)c3ccccc3)cc2)cc1 ; "IT487"]

The mixture of LIX-1 (750 mg, 5.95 mmol) and MeSO₃H (1.8 mL) in dry EtOH (5 mL) was stirred for 40 min at 150° C. in microwave. The mixture solution was removed and added water. The mixture was adjusted to pH=8 with 6M NaOH, extracted with EA. The combined organic layer was dried and concentrated to afford the crude LIX-2 (650 mg, yield: 70%) as a yellow solid.

To a mixture of NH₄Cl (19 g, 0.35 mol) and Fe (16 g, 0.28 mol) in water (200 mL) was added a solution of LIX-2 (19.4 g, 0.07 mol) in THF (100 mL)/water (100 mL) slowly at 0° C. After the addition, the reaction was stirred overnight at 60° C. The mixture was filtered and the filtrate was extracted with EA. The combined organic layers were dried over Na₂SO₄, and concentrated to give LIX-3 (14.8 g, yield 85.5%) as a brown solid.

To the solution of LIX-3 (645 mg, 4.08 mmol) in dry THF (50 mL) was slowly added NaH (171.5 mg, 4.29 mmol) at 0° C. The mixture was stirred at 25° C. for 2 hs. Then SEMC1 (712.1 g, 4.29 mmol) was slowly added to the reaction. The mixture was stirred for 18 hours at 40° C. The mixture was quenched with sat. aq. NaHCO₃ at 0° C. and extracted with EA, the combine organic was dried over Na₂SO₄, concentrated and purified to afford the LIX-4 (650 mg, yield: 55%) as a yellow oil.

To a solution of LIX-4 (700 mg, 2.43 mmol), LIX-4A (334.4 mg, 1.49 mmol) and Cs₂CO₃ (1.19 g, 3.65 mmol) in dry DMF (20 mL) was stirred at 145° C. for 2 hs. The residue was diluted with water and EA, followed standard work-up procedure and purified to afford LIX-5 (251 mg crude, yield: 23%).

The solution of LIX-5 (250 mg, 0.56 mmol), LIX-6 (250.8 mg, 0.56 mmol) and K₃PO₄.3H₂O (297.9 mg, 1.12 mmol) in 10 mL of dioxane/H₂O (10 mL, v/v=5/1) was added Pd(dtbpf)Cl₂ (18.2 mg, 0.03 mmol) under N₂. The mixture was stirred for 1 h at 90° C. Added water and extracted with EtOAc. The separated organic layers was washed with brine, dried over Na₂SO₄, concentrated and purified to afford LIX-6 (230 mg, yield: 60%).

The solution of LIX-6 (194 mg, 0.284 mmol) in 10 mL of dry dioxane was added LiBF₄ (185.4 mg, 1.99 mmol). The mixture was stirred for 21 hs at 110° C. The mixture was diluted with EtOAc and quenched with sat. aq. NaHCO₃ (15 mL). The separated organic layers were dried over Na₂SO₄, concentrated and purified to afford LIX-7 (100 mg, yield: 64%).

To a stirred solution of LIX-7 (50 mg, 0.091 mmol) in EtO H/H₂O (10 mL, v/v=5/1) was added NaOH (72.8 mg, 1.82 mmol). Then the solution was heated to 85° C. for 36 hrs. H₂O (10 mL) was added and the mixture was adjusted to pH=4 with 1M HCl and extracted DCM. The organic layer was washed with brine, dried over Na₂SO₄, concentrated and purified by Prep-HPLC to afford IT487 (2.6 mg, yield: 5.5%). MS (ESI) m/z (M+H)⁺ 525.2. ¹H NMR (Methanol-d₄, 400 MHz): δ 7.80 (br, 2H), 7.64-7.68 (m, 5H), 7.37-7.44 (m, 4H), 7.12-7.34 (m, 3H), 5.81 (d, J=5.6 Hz, 1H), 2.18 (s, 3H), 1.61 (d, J=6.8 Hz, 3H).

### Example 55

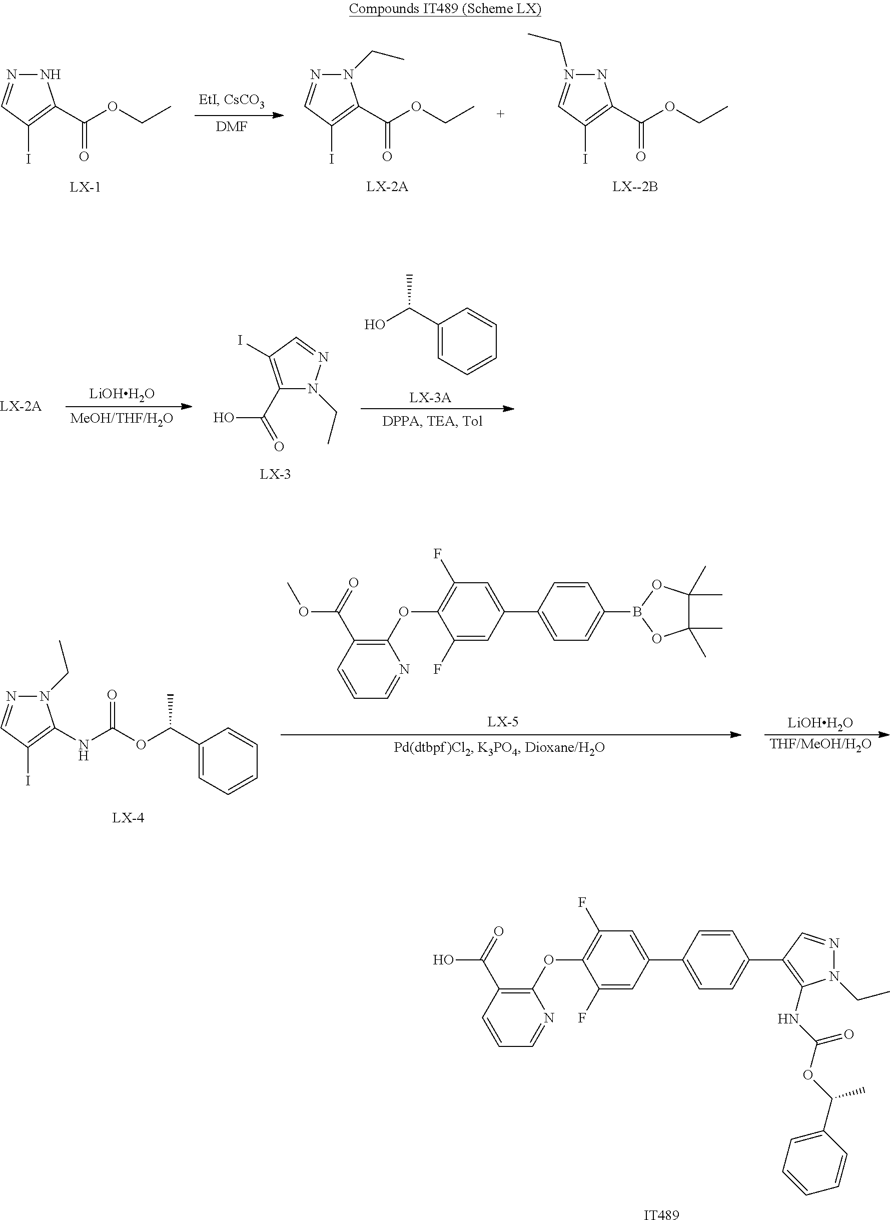

EtI (7.97 g, 51.12 mmol) was added to a solution of LX-1 (6.8 g, 25.56 mmol), Cs₂CO₃ (33.33 g, 102.24 mmol) in DMF (50 ml) was stirred at rt overnight. The mixture was washed with water, extracted with EA, dried over MgSO₄, and filtered. The combined organic layer was then concentrated and purified to afford LX-2A (4 g, yield 53%) and LX-2B (2.8 g, yield 38%).

A solution of LX-2A (2 g, 6.8 mmol), and LiOH.H₂O (2.86 g, 68 mmol) in THF/MeOH/H₂O (15 mL, v/v/v=1/1/1) was stirred at rt for 18 hrs and adjusted pH=1 with HCl (1N). The mixture was extracted with EtOAc, and the combined organic layer was subject to standard work-up procedure to afford LX-3, which was used to next step without further purification.

The mixture of LX-3 (0.6 g, 2.27 mmol), LX-3A (327 mg, 2.73 mmol), DPPA (750.75 mg, 2.73 mmol) and Et₃N (0.458 g, 4.54 mmol) in toluene (10 mL) was stirred at reflux under nitrogen for 4 hrs. The mixture was concentrated, and the residue was partitioned between H₂O and DCM, the aqueous phase was extracted with DCM. The combined organic layer was subject to standard work-up procedure and purified to afford LX-4 (385 mg, yield: 44%).

The Suzuki-coupling of LX-4 and LX-5 and the subsequent LiOH hydrolysis were conducted following the similar procedure described in Example 51 to afford IT489. Sodium salt IT489a: ¹H NMR (DMSO-d₆, 400 MHz): δ 9.62 (s, 1H), 7.72-7.90 (s, 5H), 7.58-7.60 (m, 4H), 7.30-7.40 (m, 5H), 7.03-7.06 (m, 1H), 5.75 (d, J=6.4 Hz, 1H), 3.94-3.95 (d, J=7.2 Hz, 1H), 3.81 (s, 2H), 1.53 (s, 3H), 1.28 (t, J=6.8 Hz, 3H) MS (ESI) m/z (M+H)⁺ 585.4.

### Example 56

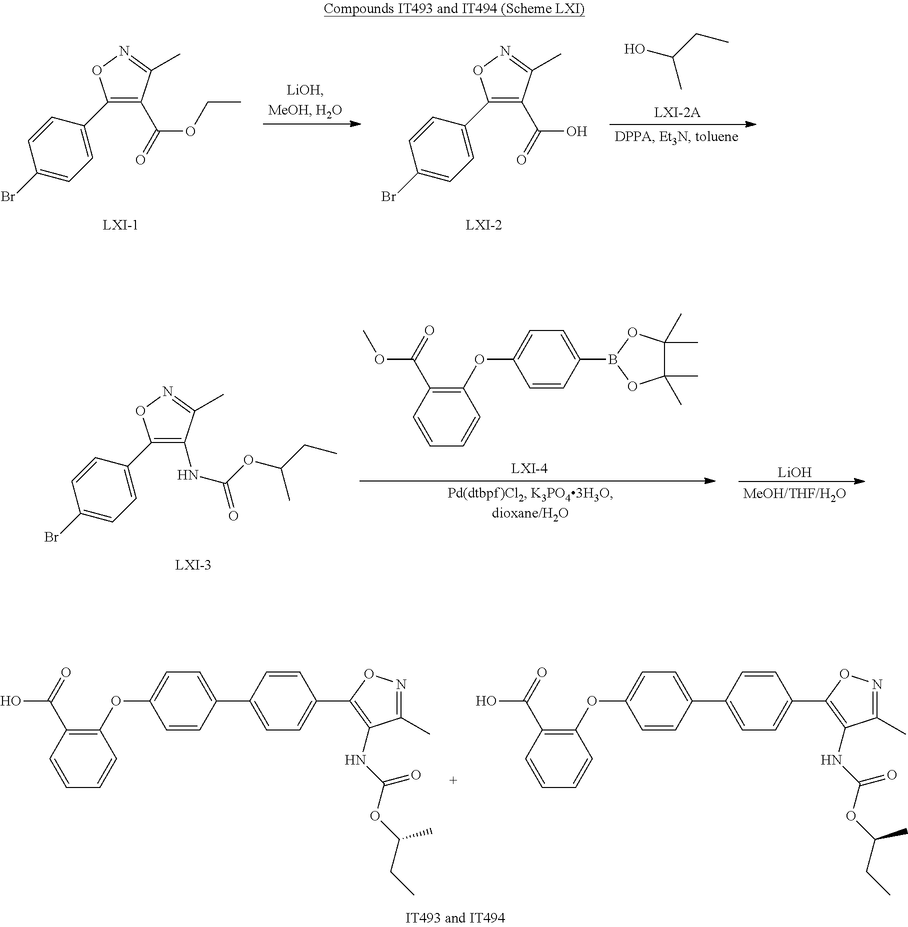

LXI-4 was prepared by reacting B1 with 4,4,4′,4′,5,5,5′,5′-octamethyl-2,2′-bi(1,3,2-dioxaborolane) following the similar procedure described in the synthesis of IT306.

LXI-3 was prepared by LiOH hydrolysis of ester LXI-1 to form the intermediate acid LXI-2, followed by reacting with butan-2-ol (LXI-2A).

IT493 and IT494 were prepared by Suzuki-coupling of LXI-3 and LXI-4, followed by LiOH hydrolysis and SFC separation. IT493: MS (ESI) m/z (M+H)⁺ 487.2. ¹H NMR (Methanol-d₄, 400 MHz): δ 7.93-7.96 (m, 3H), 7.89 (d, J=8.4 Hz, 2H), 7.75 (d, J=8.4 Hz, 2H), 7.67 (d, J=8.4 Hz, 1H), 7.27 (d, J=8.4 Hz, 1H), 7.02-7.08 (m, 3H), 4.80 (br, 1H), 2.25 (s, 3H), 1.67 (d, J=6.4 Hz, 2H), 1.30 (d, J=4.4 Hz, 3H), 1.00 (s, 3H). IT494: ¹H NMR (DMSO-d₆, 400 MHz): δ 9.11 (s, 1H), 7.84-7.88 (m, 5H), 7.82 (m, 1H), 7.32-7.34 (m, 1H), 7.13 (d, J=8.0 Hz, 1H), 6.99 (d, J=8.8 Hz, 2H), 7.02-7.08 (m, 3H), 4.69 (br, 1H), 2.17 (s, 3H), 1.59 (s, 2H), 1.23 (s, 3H), 0.92 (s, 3H).

### Example 57

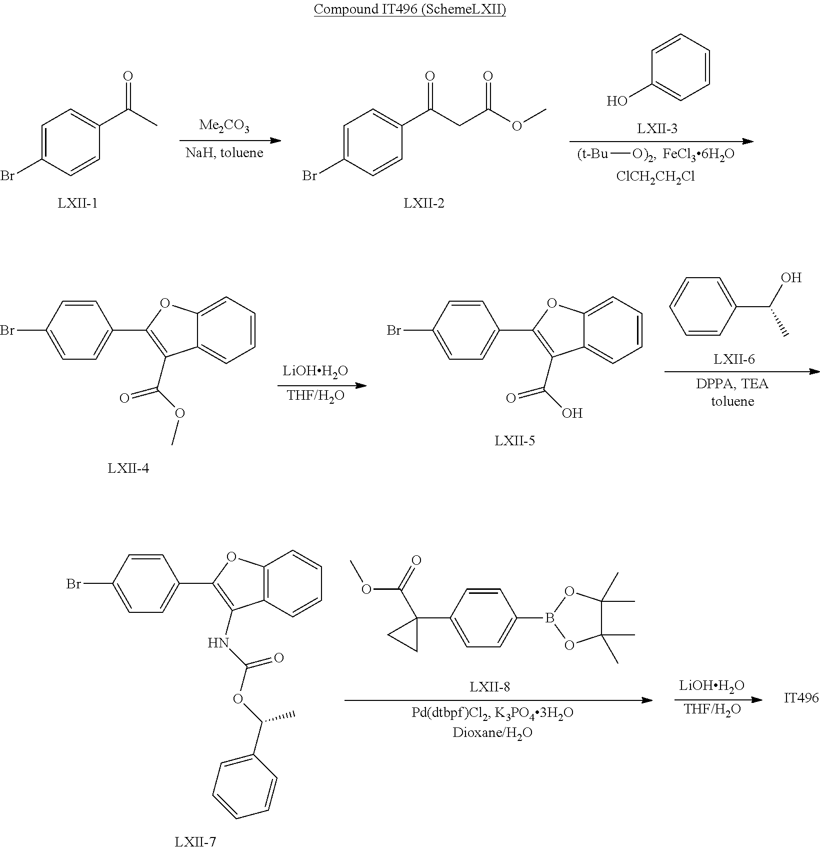

To a stirred solution of Me₂CO₃ (11 g, 126.26 mmol) in toluene (70 mL) was added NaH (4 g, 101.00 mmol) under nitrogen at rt. LXII-1 (5 g, 25.25 mmol) in toluene (30 mL) was added. Then the mixture was heated to reflux for 7 hrs. Glacial acetic acid (35 mL) wad added and this was followed by dilution with a solution of HCl in ice water. The mixture was extracted with EA. The organic layer was subject to standard work-up procedure and purified to afford LXII-2 (6.5 g, yield: 100%).

To a stirred solution of LXII-2 (6 g, 23.35 mmol), LXII-3 (6.6 g, 70.04 mmol), FeCl₃.6H₂O (0.63 g, 2.33 mmol) in DCE (50 mL) was added (t-BuO)₂ (6.8 g, 46.70 mmol) under nitrogen. Then the mixture was heated to 100° C. for 4 hrs, then quenched with saturated NaHCO₃ and extracted with EA. The combined organic layer was subject to standard work-up procedure and purified to afford LXII-4 (2.8 g, yield: 36.4%).

To a stirred solution of LXII-4 (2 g, 6.04 mmol) in 30 mL of THF/H₂O (v/v=5:1) was added LiOH H₂O (254 mg, 30.21 mmol). Then the mixture was heated to 60° C. for 2 hrs. THF was removed in vacuo, H₂O was added and the residue was adjusted to pH=3˜4 with aq. HCl (1M). The aqueous phase was extracted with DCM. The combined organic layer was subject to standard work-up procedure to afford LXII-5 (1.33 g, yield 69.2%).

To a stirred solution of LXII-5 (1.33 g, 4.20 mmol), DPPA (1.4 g, 5.04 mmol), Et₃N (848 mg, 8.40 mmol) in toluene (30 mL) was added LXII-6 (615 mg, 5.04 mmol) under nitrogen. Then the solution was heated to 90° C. for 2 hs. H₂O was added and the mixture was extracted with EA. The organic layer was subject to standard work-up procedure and purified to afford LXII-7 (1 g, yield: 54.6%).

IT496 was prepared by Suzuki-coupling of LXII-7 with LXII-8, followed by LiOH hydrolysis. MS (ESI) m/z (M−H)⁺ 518.2. ¹H NMR (DMSO-d₆, 400 MHz): δ 12.39 (s, 1H), 9.57 (s, 1H), 7.93 (d, J=6.8 Hz, 2H), 7.77 (d, J=6.4 Hz, 2H), 7.62-7.67 (m, 3H), 7.44-7.46 (m, 6H), 7.35-7.38 (q, J=7.4 Hz, 2H), 7.26-7.30 (q, J=7.4 Hz, 2H), 5.80 (s, 1H), 1.60 (s, 3H), 1.48-1.49 (m, 2H), 1.17-1.20 (m, 2H).

IT495 was prepared by Suzuki-coupling of LXI-4 with LXII-7, followed by LiOH hydrolysis to afford the final product. ¹H NMR (DMSO-d₆, 400 MHz): δ 12.95 (s, 1H), 9.58 (s, 1H), 7.87-7.92 (m, 3H), 7.72-7.74 (m, 4H), 7.60-7.64 (m, 2H), 7.44-7.47 (m, 4H), 7.26-7.40 (m, 4H), 7.14 (d, J=8.0 Hz, 1H), 7.01 (d, J=8.4 Hz, 2H), 5.80 (s, 1H), 1.59 (s, 3H).

### Wxample 58

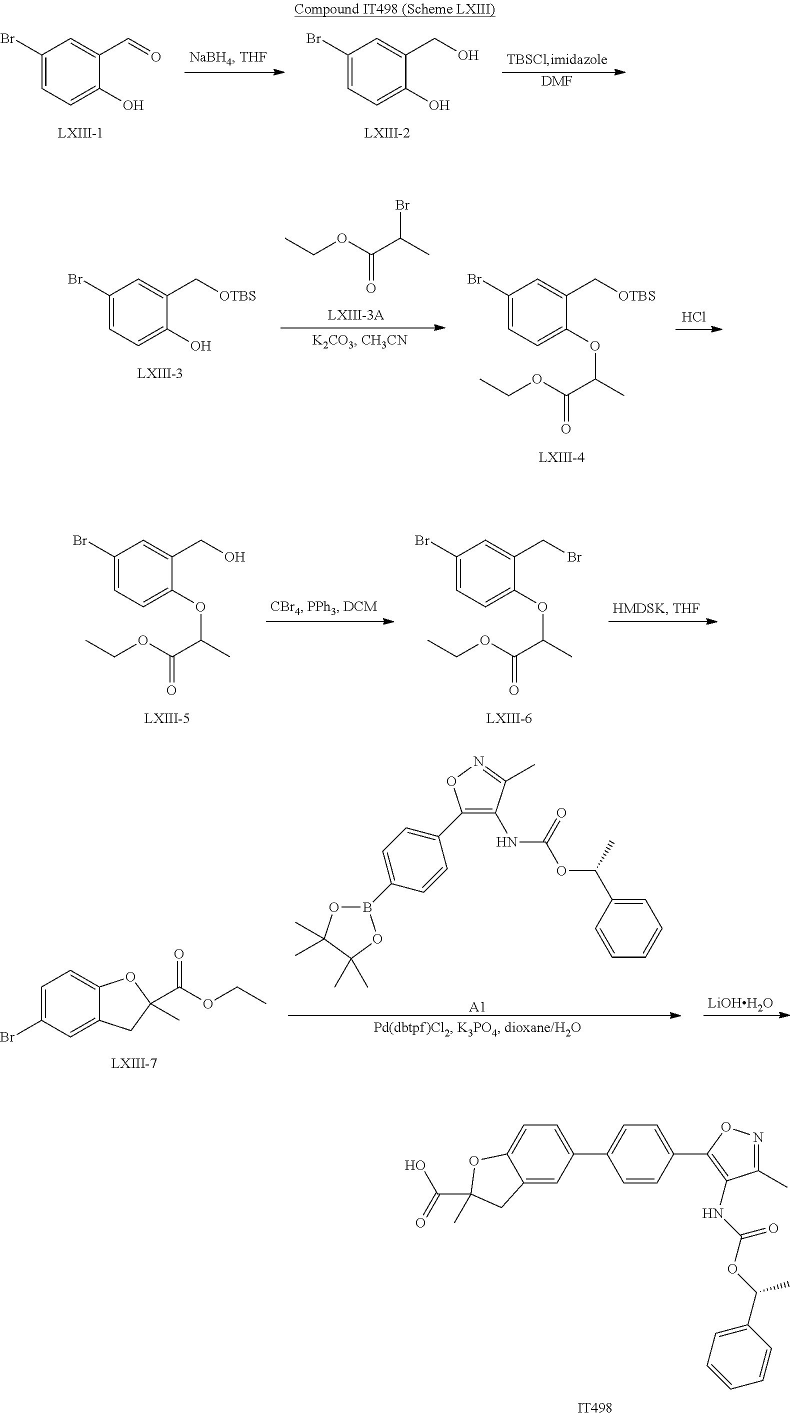

To a stirred solution of LXIII-1 (8 g, 0.04 mol) in THF (100 mL) was added NaBH₄ (2.28 g, 0.06 mol) at 0° C. under N₂. The mixture was stirred at rt for 2 hrs. It was quenched with sat. aq. NH₄Cl and extracted with EA. The combined organic layers were washed with brine, and concentrated under vacuo to give LXIII-2 (8 g, crude, yield: 100%).

To a stirred solution of LXIII-2 (4 g, 20 mmol) and imidazole (1.32 g, 20 mmol) in DMF (100 mL) was added TBSC1 (3.24 g, 20 mmol) under N₂. The mixture was stirred at rt for 5 hrs. The mixture was diluted with water and extracted with EA. The combined organic layers were subject to standard work-up procedure and purified to give LXIII-3 (4 g, yield: 64%).

To a stirred solution of LXIII-3A (2.96 g, 18.9 mmol) and LXIII-3 (4 g, 12.6 mmol) in CH₃CN (50 mL) was added K₂CO₃ (3.5 g, 25.3 mmol) under N₂. The mixture was heated to 70° C. for 2 hrs. After being cooled to rt, the mixture was diluted with water and extracted with DCM. The combined organic layers were subject to standard work-up procedure and purified to give LXIII-4 (3.2 g, yield: 61%), which was further deprotected by sat. aq. HCl to afford LXIII-5.

To a stirred solution LXIII-5 (2.3 g, 7.6 mmol) in 50 mL of DCM was added CBr₄ (5 g, 15.2 mmol) and PPh₃ (2.5 g, 9.1 mmol). The mixture was stirred at rt for 1 h. It was washed with sat. aq. NaHCO₃ and water, dried and concentrated. The residue was purified to give LXIII-6 (1.7 g, yield: 61%).

To a stirred solution of LXIII-6 (1 g, 2.75 mmol) in 20 mL of THF was added HMDSK (4.12 mL, 4.12 mmol) at −70° C. under nitrogen. The mixture was stirred at rt for 3 hrs. The solution was quenched with sat. NH₄Cl and extracted with EA. The combined organic layer was subject to standard work-up procedure and purified to give LXIII-7 (450 mg, yield: 58%).

IT498 was prepared by Suzuki-coupling of LXIII-7 with A1 (Example 51), followed by LiOH hydrolysis to afford the final product. MS (ESI) m/z (M+H)⁺ 499.3. ¹H NMR (Methanol-d₄, 400 MHz): δ 7.79 (d, J=7.6 Hz, 2H), 7.64 (d, J=8.4 Hz, 2H), 7.39-7.51 (m, 6H), 7.19-7.33 (m, 1H), 6.90 (d, J=8.0 Hz, 1H), 5.82-5.84 (m, 1H), 3.67-3.71 (d, J=16 Hz, 1H), 3.24-3.28 (d, J=16.4 Hz, 1H), 2.19 (s, 3H), 1.72 (s, 3H), 1.62 (d, J=6.4 Hz, 3H).

### Example 59

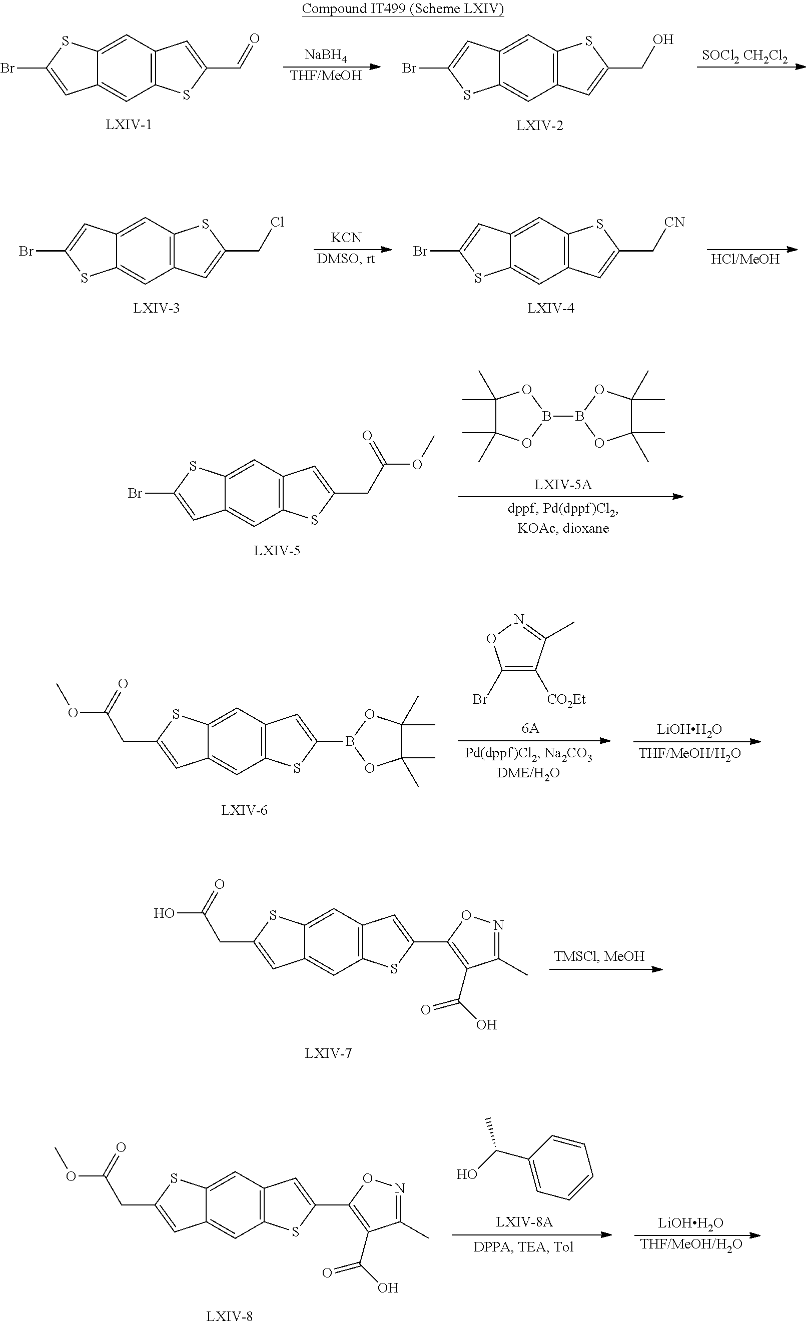

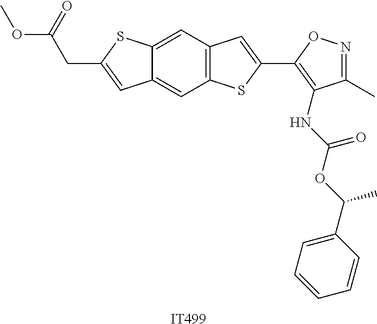 [SMILES: COC(=O)Cc1cc2cc3sc(-c4onc(C)c4NC(=O)O[C@H](C)c4ccccc4)cc3cc2s1 ; "IT499"]

The preparation of LXIV-1 was described in the synthesis of IT080 (Example 34).

A solution of LXIV-1 (6.6 g, 22.21 mmol) in 80 mL of THF/MeOH (v/v=3/1) was cooled to 0° C. NaBH₄ (2.1 g, 55.53 mmol) was added in portions. After addition, the mixture was stirred for 2 hrs at 0° C. The mixture was diluted with H₂O, extracted with EA. The combined organic layer was subject to standard work-up procedure and purified to afford LXIV-2 (4.4 g, yield 69%) as a pale yellow solid.

A mixture of LXIV-2 (4.4 g, 14.71 mmol) in 70 mL of dry dichloromethane was cooled to 0° C. SOCl₂ (10.6 mL, 147.05 mmol) was added slowly. The mixture was stirred for 2 hrs at 0° C. The mixture was evaporated. The residue was azeotropied twice with toluene to afford LXIV-3 (4.7 g, 100% crude yield) as a white-off solid.

To a mixture of LXIV-3 (2.7 g, 8.50 mmol) in 40 mL of DMSO was added KCN (1.1 g, 17.00 mmol) at 0° C. The mixture was stirred for 3 hrs at rt. The mixture was diluted with H₂O, extracted with EA. The combined organic layer was subject to standard work-up procedure and purified to afford LXIV-4 (1.0 g, yield 38%) as a yellow solid.

To a mixture of compound 4 (1.2 g, 3.89 mmol) in 6 mL of dry methanol was added 4 N HCl in methanol. The mixture was heated to reflux overnight. The mixture was concentrated. The residue was dissolved in EA, washed and concentrated. The residue was purified to afford LXIV-5 (1.2 g, yield 92%).

LXIV-6 was prepared following the similar procedure described in the synthesis of XLII-8 by reacting LXIV-5 with LXIV-5A.

Argon gas was bubbled through a mixture of LXIV-6 (600 mg, 1.56 mmol) and LXIV-6A (580 mg, 2.46 mmol) in 6 mL of DME/H₂O (v/v=3/1). Then Na₂CO₃ (327 mg, 3.09 mmol) was added, followed by Pd(dppf)Cl₂ (114 mg, 0.16 mmol). The mixture was stirred at 90° C. for 30 mins under microwave condition. The mixture diluted with DCM, filtered through Celite, the filtrate was washed with brine, dried and concentrated. The residue was purified and subjected to LiOH hydrolysis to afford LXIV-7.

A suspension of LXIV-7 (60 mg, 0.16 mmol) in 4 mL of methanol was cooled to 0° C. TMSCl (102 uL, 0.80 mmol) was added. The mixture was stirred for 5 hrs at rt. The mixture was diluted with 5 mL of H₂O, extracted with EA. The combined organic layer was washed with brine, dried and concentrated. The residue was purified to afford LXIV-8 (35 mg, yield 56%) as a yellow solid.

To a suspension of LXIV-8 (50 mg, 0.13 mmol) in 2 mL of toluene was added TEA (36 uL, 0.26 mmol). The mixture turned clear. Then DPPA (41 mg, 0.15 mmol) was added, followed by LXIV-8A (19 mg, 0.15 mmol). The mixture was heated to 80° C. and stirred for 4 hrs. The mixture was concentrated and the residue was purified as a pale yellow solid, which was subsequently hydrolyzed by LiOH to afford the final product IT499. ¹H NMR (400 MHz, Methanol-d₄): δ 8.62 (s, 1H), 8.29 (s, 1H), 7.90 (s, 1H), 7.28-7.36 (m, 5H), 7.06 (br, 1H), 5.76 (br, 1H), 3.96 (s, 2H), 2.23 (s, 3H), 1.55 (d, J=5.6 Hz, 3H). MS (ESI) m/z (M−H)⁻ 491.0.

### In Vitro Assays

Establishment of a CHO Cell Line Stably Expressing Human LPA₁

A 1.1 kb cDNA encoding the human LPA₁ receptor is cloned from human lung. Human lung RNA (Clontech Laboratories, Inc. USA) is reverse transcribed using the RETROscript kit (Ambion, Inc.) and the full-length cDNA for human LPA₁ is obtained by PCR of the reverse transcription reaction. The nucleotide sequence of the cloned human LPA₁ is determined by sequencing and is confirmed to be identical to the published human LPA₁ sequence (An et al. Biochem. Biophys. Res. Commun. 231:619 (1997). The cDNA is cloned into the pCDNA5pcDNA5/FRT expression plasmid and is transfected in CHO cells using lipofectamine 2000 (Invitrogen Corp., USA). Clones stably expressing human LPA₁ are selected using hygromycin and identified as cells that show Ca-influx in response to LPA.

Generation of Cells Transiently Expressing Human LPA₂

A vector containing the human LPA₂ receptor cDNA is obtained from the Missouri S&T cDNA Resource Center (www.cdna.org). The full-length cDNA fragment for human LPA₂ is obtained by PCR from the vector. The nucleotide sequence of the cloned human LPA₂ is determined by sequencing and is confirmed to be identical to the published human LPA₂ sequence (NCBI accession number NM_{—}004720). The cDNA is cloned into the pCDNA3pcDNA3.1 expression plasmid and is transfected into B103 cells (Invitrogen Corp., USA) by seeding cells in a 96-well poly-D-lysine coated plate at 30,000-35,000 cells per well together with 0.2 μl lipofectamine 2000 and 0.2 μg of the LPA₂ expression vector. Cells are cultured overnight in complete media before being assayed for LPA-induced Ca-influx.

Establishment of a CHO Cell Line Stably Expressing Human LPA₃

A vector containing the human LPA₃ receptor cDNA is obtained from the Missouri S&T cDNA Resource Center (www.cdna.org). The full-length cDNA fragment for human LPA₃ is obtained by PCR from the vector. The nucleotide sequence of the cloned human LPA₃ is determined by sequencing and is confirmed to be identical to the published human LPA₃ sequence (NCBI accession number NM_{—}012152). The cDNA is cloned into the pCDNA5pcDNA5/FRT expression plasmid and is transfected in CHO cells using lipofectamine 2000 (Invitrogen Corp., USA). Clones stably expressing human LPA₃ are selected using hygromycin and identified as cells that show Ca-influx in response to LPA.

LPA1 and LPA3 Calcium Flux Assays

Human LPA₁ or LPA₃ expressing CHO cells were seeded at 20,000-45,000 cells per well in a 96-well poly-D-lysine coated plate one or two days before the assay. Prior to the assay, the cells were washed once with PBS and then cultured in serum-free media overnight. On the day of the assay, a calcium indicator dye (Calcium 4, Molecular Devices) in assay buffer (HBSS with Ca²⁺ and Mg²⁺ and containing 20 mM Hepes and 0.3% fatty-acid free human serum albumin) was added to each well and incubation continued for 1 hour at 37° C. 10 μl of test compounds in 2.5% DMSO were added to the cells and incubation continued at room temperature for 30 minutes. Cells were then stimulated by the addition of 10 nM LPA and intracellular Ca²⁺ measured using the Flexstation 3 (Molecular Devices). IC₅₀ₛ were determined using Graphpad prism analysis of drug titration curves.

LPA2 Calcium Flux Assay

Following an overnight culture with lipofectamine 2000 and the LPA₂ expression vector, the B103 cells are washed once with PBS then serum starved for 4 hours. A calcium indicator dye (Calcium 4, Molecular Devices) in assay buffer (HBSS with Ca²⁺ and Mg²⁺ and containing 20 mM Hepes and 0.3% fatty-acid free human serum albumin) is added to each well and incubation continued for 1 hour at 37° C. 10 μl of test compounds in 2.5% DMSO are added to the cells and incubation continued at room temperature for 30 minutes. Cells are the stimulated by the addition of 10 nM LPA and intracellular Ca²⁺ measured using the Flexstation 3 (Molecular Devices). IC₅₀ₛ are determined using Graphpad prism analysis of drug titration curves.

GTPγS Binding Assay

The ability of a compound to inhibit binding of GTP to LPA₁ is assessed via a membrane GTPγS assay. CHO cells stably expressing the recombinant human LPA₁ receptor are resuspended in 10 mM Hepes, 7.4 containing 1 mM DTT, lysed and centrifuged at 75,000×g to pellet the membranes. The membranes are resuspended in 10 mM Hepes, 7.4 containing 1 mM DTT and 10% glycerol. Membranes (˜(−25 μg per well) are incubated in 96-well plates with 0.1 nM [³⁵S]-GTPγS, 900 nM LPA, 5 μM GDP, and test compound in Assay Buffer (50 mM Hepes, pH 7.4, 100 mM NaCl, 10 mM MgCl₂, 50 μg/ml saponin and 0.2% fatty-acid free human serum albumin) for 30 minutes at 30° C. The reactions are terminated by rapid filtration through Whatman GF/B glass fiber filter plates. The filter plates are washed 3 times with 1 ml cold Wash Buffer (50 mM Hepes, 7.5, 100 mM NaCl and 10 mM MgCl₂) and dried. Scintillant is then added to the plates and the radioactivity retained on the filters is determined on a Packard TopCount (Perkin Elmer). Specific binding is determined as total radioactive binding minus non-specific binding in the absence of the ligand (900 nM LPA). IC₅₀ₛ are determined using Graphpad prism analysis of drug titration curves.

Beta-Arrestin Based Assays for Human LPA1R Antagonists and Agonists

A CHO cell line stably expressing the ProLink™ tagged human LPA1R was obtained from DiscoverX Inc, Fremont, Calif. In this system, β-Arrestin was fused to an N-terminal deletion mutant of β-galactosidase (termed the enzyme acceptor or EA), the human LPA1R was fused to a smaller (42 amino acids) weakly complementing fragment termed ProLink™. In cells that stably express these fusion proteins, agonist/ligand stimulation resulted in the interaction of β-Arrestin and the ProLink-tagged GPCR, forcing the complementation of the two β-galactosidase fragments and resulting in the formation of a functional enzyme that converted substrate to detectable signal. Cell handling and assays were performed according to protocols specified in the PathHunter® assays kits (DiscoverX, Fremont, Calif.). Assays were performed in quadruplicate in white 384 well plates. End point luminescence data were plotted and fit to a 4 parameter logistic function to obtain IC₅₀ values. For antagonist assays, an IC₈₀ concentration of agonist (LPA) equal to 0.5 micromolar was used.

Beta-Arrestin Based Assays for LPA and S1P Receptor Antagonists and Agonists (Human and Species Orthologs) Using Transiently Transfected Cells

CMV promoter based DNA constructs expressing a fusion of the LPA/S1P GPCR of interest and ProLink™ tag were used to transfect EA Parental™ CHO cells (DiscoverX, Fremont, Calif.) using a FuGENE® transfection kit (Roche). Beta-Arrestin based assays were conducted 24-48 hrs post transfection using PathHunter® assay kits (DiscoverX, Fremont, Calif.). Agonist and antagonist assays were performed in quadruplicate in white 384 well plates. End point luminescence data were plotted and fit to a 4 parameter logistic function to obtain IC₅₀ values. For antagonist assays, an IC₈₀ concentration of agonist (LPA) equal to 0.5 micromolar was used.

cAMP Based Assays for Human LPA1R Antagonists and Agonists

A CHO cell line stably expressing the human LPA1R (DiscoverX Inc, Fremont, Calif.) was used according to manufacturer's protocol. HitHunter® assay kits (DiscoverX, Fremont, Calif.) were used to measure cAMP levels. HitHunter® cAMP assays are competitive immunoassays. Free cAMP from cell lysates competed for antibody binding against labeled cAMP (ED-cAMP conjugate). Unbound ED-cAMP was free to complement EA to form active enzyme, which subsequently hydrolyzed substrate to produce signal. A positive signal generated was directly proportional to the amount of free cAMP bound by the binding protein. Forskolin (15 micromolar) was used to elevate cAMP levels. Increased LPA (agonist) activity was measured as a decrease in cAMP levels. For antagonist assays, an IC₈₀ of LPA (agonist) equal to 50 micromolar was used, and increased antagonist activity of the test compound was recorded as an increase in cAMP levels. All assays were performed in quadruplicate in white 384 well plates. End point luminescence data were plotted and fit to a 4 parameter logistic function to obtain IC₅₀ values.

LPA1 Chemotaxis Assay

Chemotaxis of the A2058 human melanoma cells is measured using the Neuroprobe ChemoTx® System plates (8 μm pore size, 5.7 mm diameter sites). The filter sites are coated with 0.001% fibronectin (Sigma) in 20 mM Hepes, pH 7.4 and allowed to dry. A2058 cells are serum-starved for 24 hours, then are harvested with Cell Stripper and are resuspended in DMEM containing 0.1% fatty-acid-free bovine serum albumin (BSA) to a concentration of 1.times.10.sup.6/ml. Cells are mixed with an equal volume of test compound (2x) in DMEM containing 0.1% fatty-acid-free BSA and incubated at 37° C. for 15 minutes. LPA (100 nM in DMEM containing 0.1% fatty-acid-free BSA) or vehicle is added to each well of the lower chamber and 50 μl of the cell suspension/test compound mix is applied to the upper portion of the ChemoTx® plate. Plates are incubated at 37° C. for three hours and then the cells are removed from the upper portion by rinsing with PBS and scraping. The filter is dried then stained with HEMA 3 Staining System (Fisher Scientific). The absorbance of the filter is read at 590 nM and IC₅₀ₛ are determined using Symyx Assay Explorer.

LPA1 Migration Assay

Migration of primary fibroblasts (including lung, dermal), HFL-1, 3T3 and CHO cells expressing LPA1R were monitored using the Oris™ assay (Platypus Technologies, Madison, Wis.). These cells were dye (Cell Tracker Green™) loaded and serum starved for 12-24 hrs. In response to chemoattractants such as LPA and serum, the cells migrated inward in to the exclusion (detection) zone. After fixing, fluorescent cells in the detection zone were counted using a high content reader. The ability of LPA1 antagonists to inhibit cell migration is quantified by plotting cell number vs. compound concentration and curve fitting the resulting dose-response curve to a 4 parameter logistic function.

Assay of Inhibitory Effect on Cell Proliferation (CM Thymidine Incorporation)

Fibroblasts (primary human lung and dermal, HFL-1, 3T3 etc) are plated on a 96-well plate and serum starved for 24-48 hours. The media are then exchanged for media containing stimulants (LPA, TGFb, serum etc) and cultured further for 16-24 hours before [³H] thymidine addition. After culturing for another 8 hours, cells are washed with PBS and the amount of [³H] thymidine incorporated into the cells are assayed by Betaplate filter counter system (Amersham Pharmacia Biotech). The difference between the amount of [³H] thymidine incorporated in the stimulant-added well and the amount of [³H] thymidine incorporated in the well containing no stimulant represents the amount of [³H] thymidine incorporation accelerated by stimulant. The increase of [³H] thymidine incorporation without the addition of test compounds is set as 100% and the concentration of compound with 50% inhibition in the increase of [³H] thymidine incorporation (IC₅₀ value) is determined. The test compounds are added 0-30 min before stimulant addition.

Assay of Inhibitory Effect on Cell Proliferation (BrdU Incorporation)

Fibroblasts (primary human lung and dermal, HFL-1, 3T3 etc) were plated on a 96-well plate and serum starved for 24-48 hours. The media were then exchanged for media containing stimulants (LPA, TGFb, serum etc) and cultured further for 16-24 hours before BrdU addition. After culturing for another 8 hours, cells were washed with PBS and the amount of BrdU incorporated into the cells was assayed by absorbance at 450 nm using the Cell proliferation ELISA system (RPN250, Amersham LIFE SCIENCE). The difference between the amount of BrdU incorporated in the stimulant-added well and the amount of BrdU incorporated in the well containing no stimulant represented the amount of BrdU incorporation accelerated by stimulant. The increase of BrdU incorporation without the addition of test compounds was set as 100% and the concentration of compound with 50% inhibition in the increase of BrdU incorporation (IC₅₀ value) was determined. The test compounds were added 0-30 min before stimulant addition.

Myofibroblast Differentiation

Fibroblasts (primary human lung and dermal, HFL-1, 3T3 etc) are plated on a 96-well plate and serum starved for 24-48 hours. The media are then exchanged for media containing stimulants (LPA, TGFb, etc) and cultured further for 24-48 hours. The amount of alpha smooth muscle actin (aSMA) is quantitated using an ELISA kit (Thermo Scientific, USA). Alternatively after fixing and permeabilization, aSMA is also quantitated using immunohistochemical methods (FITC conjugated anti-aSMA, Sigma).

Assay for Effect of Compounds on Collagen Production

HFL-1 Cells (ATCC, Rockville, Md.) are grown under regular tissue culture conditions in complete media containing 10% fetal bovine serum (FBS; Mediatech, Inc. Herndon, Va.). Cells in early passage are plated in 6 well plates. When the cells reach confluence, the media is removed, cells are washed with PBS, and the cells are kept overnight in complete media containing 0.1% FBS. The media is then replaced with fresh media plus 0.1% FCS, 10 flM L-Proline (EMD Chemicals, Gibbstown, N.J.), 20 flg/mL ascorbic acid (EMD Chemicals, Gibbstown, N.J.). Compounds are added to triplicate wells to a final concentration of 1 mM from 100× stock solutions in DMSO. One hour after the addition of compound, the cells are treated with TGFb (Sigma-Aldrich, St. Louis, Mo.) to a final concentration of 10 ng/mL (25 ng total). Three days after addition of TGFb the media is removed, cells are washed with PBS and then lysed. The total collagen content of lysed cells is assessed with a dye-based collagen assay (Sircol Collagen Assay, Newtownabbey, Northern Ireland) and an flQuant plate-based spectrophotometer (BioTek Instruments, Inc., Winooski, Vt.) with appropriate standard curves. The dynamic range of the assay is defined by cells that were mock treated (1% DMSO without compound) in the presence and absence of TGFb.

Bleomycin-Induced Lung Fibrosis Model in Mice or Rats

Female C57B1/6CD-1 mice (Harlan, 25-30 g) or Wistar rats (Harlan, 200-250 g) are housed 4 per cage, given free access to food and water and allowed to acclimate for at least 7 days prior to test initiation. After the habituation phase, animals are lightly anesthetized with isoflurane (5% in 100% O₂) and administered with bleomycin sulfate (Henry Schein) via intratracheal instillation (Cuzzocrea S et al. Am J Physiol Lung Cell Mol. Physiol. 2007 May; 292(5):L1095-104. Epub 2007 Jan. 12.). Animals are returned to their cages and monitored daily for the duration of the experiment. Test compound or vehicle is delivered po, ip, or sc daily. The route and frequency of dosing is based on previously determined pharmacokinetic properties. All animals are sacrificed using inhaled isoflurane 3, 7, 14, 21 or 28 days after bleomycin instillation. Following sacrifice, animals are intubated with a 20 gauge angiocatheter attached to a 1 ml syringe. Lungs are lavaged with saline to obtain bronchoalveolar lavage fluid (BALF) and then removed and fixed in 10% neutral buffered formalin for subsequent histopathological analysis. BALF is centrifuged for 10 min at 800×g to pellet the cells and the cell supernatant removed and frozen at −80° C. for subsequent protein analysis using the DC protein assay kit (Biorad, Hercules, Calif.) and soluble collagen analysis using Sircol (Biocolor Ltd, UK). BALF is analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor and lactate dehydrogenase activity, using commercially available ELISA. The cell pellet is re-suspended in PBS. Total cell counts are then obtained using a Hemavet hematology system (Drew Scientific, Wayne, Pa.) and differential cells counts are determined using Shandon cytospin (Thermo Scientific, Waltham, Mass.). Lung tissue is stained using hematoxylin and eosin (H&E) and trichrome and lung fibrosis is determined by semiquantitative histopathological scoring (Ashcroft T. et al. J. Clin. Path. 1988; 41; 4, 467-470) using light microscopy (10× magnification) and quantitative, computer-assisted densitometry of collagen in lung tissue sections using light microscopy. The data are plotted using Graphpad prism and statistical differences between groups determined.

Mouse Carbon Tetrachloride (CCl4)-Induced Liver Fibrosis Model

Female C57BL/6 mice (Harlan, 20-25 g) housed 4/cage are given free access to food and water and allowed to acclimate for at least 7 days prior to test initiation. After the habituation phase, mice receive CC1.sub.4 (0.5-1.0 ml/kg body weight) diluted in corn oil vehicle (100 μL volume) via i.p. injection twice a week for 84-6 weeks. (Higazi, A. A. et al., Clin Exp Immunol. 2008 April; 152(1):163-73. Epub 2008 Feb. 14). Control mice receive an equivalent volume of corn oil vehicle only. Test compound or vehicle is delivered po, ip, or sc daily. At the end of the study (8 weeks after first i.p. injection of CCl₄), mice are sacrificed using inhaled isoflurane and blood is drawn via cardiac puncture for subsequent analysis of ALT/AST levels. The liver is harvested, and one half of the liver is frozen at −80° C. and the other half is fixed in 10% neutral buffered formalin for histological assessment of liver fibrosis using light microscopy (10× magnification). Liver tissue homogenates are analyzed for collagen levels using Sircol (Biocolor Ltd, UK). Fixed Liver tissue is stained using hematoxylin and eosin (H&E) and trichrome and liver fibrosis is determined by quantitative, computer-assisted densitometry of collagen in liver tissue sections using light microscopy. Plasma and liver tissue lysates are also analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor, and lactate dehydrogenase activity, using commercially available ELISA. The resulting data are plotted using Graphpad prism and statistical differences between groups determined.

Mouse Intravenous LPA-Induced Histamine Release

A mouse intravenous LPA-induced histamine release model is utilized to determine the in vivo potency of LPA₁ and LPA₃ receptor antagonists. Female CD-1 mice (weighing 25-35 grams) are administered compound (i.p., s.c. or p.o.) in a volume of 10 ml/kg 30 minutes to 24 hours prior to intravenous LPA challenge (300 μg/mouse in 0.1% FAF BSA). Immediately following LPA challenge mice are placed into an enclosed Plexiglas chamber and exposed to an isoflurane for a period of 2-10 minutes. They are removed, and blood collected into tubes containing EDTA. Blood is then centrifuged at 10,000×g for 10 minutes at 4° C. Histamine concentrations in the plasma are determined by EIA. Drug concentrations in plasma are determined by mass spectrometry. The dose to achieve 50% inhibition of blood histamine release is calculated by nonlinear regression (Graphpad Prism) and plotted as the ED₅₀. The plasma concentration associated with this dose is plotted as the EC₅₀.

Mouse Unilateral Ureteral Obstruction Kidney Fibrosis Model

Female C57BL/6 mice (Harlan, 20-25 g) housed 4/cage will be given free access to food and water and allowed to acclimate for at least 7 days prior to test initiation. After the habituation phase, mice undergo unilateral ureteral obstruction (UUO) surgery or sham to left kidney. Briefly, a longitudinal, upper left incision is performed to expose the left kidney. The renal artery is located and 6/0 silk thread is passed between the artery and the ureter. The thread is looped around the ureter and knotted 3 times insuring full ligation of ureter. The kidney is returned to abdomen, the abdominal muscle is sutured and the skin is stapled closed. Mice are returned to their cages and monitored daily for the duration of the experiment. Test compound or vehicle is delivered po, ip, or sc daily. The route and frequency of dosing is based on previously determined pharmacokinetic properties. All animals are sacrificed using inhaled isoflurane 4, 8, 14, 21, or 28 days after UUO surgery. Following sacrifice blood is drawn via cardiac puncture, the kidneys are harvested and one half of the kidney is frozen at −80° C. and the other half is fixed in 10% neutral buffered formalin for histological assessment of kidney fibrosis using light microscopy (10× magnification). Kidney tissue homogenates are analyzed for collagen levels using Sircol (Biocolor Ltd, UK). Fixed kidney tissue is also stained using hematoxylin and eosin (H&E) and trichrome and kidney fibrosis is determined by quantitative, computer-assisted densitometry of collagen in liver tissue sections using light microscopy and collagen content in kidney lysate. Plasma and kidney tissue lysates are also analyzed for concentrations of inflammatory, pro-fibrotic and tissue injury biomarkers including transforming growth factor β1, hyaluronic acid, tissue inhibitor of metalloproteinase-1, matrix matelloproteinase-7, connective tissue growth factor and plasminogen activator inhibitor-1 lactate dehydrogenase activity, using commercially available ELISA. The resulting data are plotted using Graphpad prism and statistical differences between groups determined.

Mouse Dermal Vascular Leak Assay

Female BALB/c mice (Harlan) weighing 20-25 grams are given free access to standard mouse chow and water and are allowed to acclimate for two weeks prior to study initiation. Compounds are prepared in at a range of concentrations and delivered by oral gavage. Three hours following dose, mice are placed into a restraining device and given Evan's blue dye intravenously by tail vein injection (0.2 ml of a 0.5% solution). Mice are then anesthetized using 3% isoflurane anesthesia to allow for intradermal injection of LPA (30 μg in 20 μll 0.1% fatty acid free BSA). Thirty minutes after LPA injection mice are sacrificed by CO₂ inhalation and the skin is removed from the challenge site and placed into 2 ml formamide for overnight extraction of Evan's blue dye. Following extraction, a 150 μl aliquot of formamide for each tissue sample is placed into a 96 well plate and read at 610 nm using a photospectrometer. The resulting data (OD units) are plotted using GraphPad Prizm.

Bleomycin Dermal Fibrosis Model

Bleomycin is dissolved in phosphate buffered saline (PBS) at 10 ug/ml, and sterilized by filtration. Bleomycin or PBS control (100 ul) is injected subcutaneously into two locations on the shaved back of C57/BL6 or 5129 mice (Charles River/Harlan Labs, 20-25 g) once daily for 28 days while under isoflourane anesthesia (5% in 100% O₂). Test compounds or controls are administered throughout the study via subcutaneous or intraperitoneal injection, or via oral gavage. After 28 days, mice are euthanized and 6 mm-full thickness punch biopsies are obtained from each injection site. Dermal fibrosis is assessed by histopathology and hydroxyproline biochemical assays.

Rat Dermal Wound Healing

Female rats (Harlan Labs, 200-250 g) are given a single 1 cm-full thickness incisional wound on the back while under isoflourane anesthesia. The incision is placed parallel to the midline along the dorsal skin, using a surgical scalpel. For excisional wounds, an 8 mm-full thickness skin biopsy punch is made on the back of each animal opposite to the site of the incision. Test compounds are administered prior to wounding, and dosed for 14 days. Wounds are allowed to heal, and photographs are taken and analyzed digitally to measure wound healing throughout the study. At the end of the study animals are euthanized and wound closure determined.

### Assay Data for Compounds

Compounds of the preferred embodiments were prepared according to the methods described herein and assay data obtained for Beta Arrestin EC₅₀ assay, cell migration EC₅₀ assay, and Ca Flux LPA1 IC₅₀ assay. Control compounds were also prepared and assay data obtained. The assay data obtained for Beta Arrestin EC₅₀ assay, cell migration EC₅₀ assay and Ca Flux LPA1 IC₅₀ assay are presented in Tables 16, 17 and 18 respectively, in which A=greater than 500 nM, B=greater than or equal to 50 nM and less than or equal to 500 nM; and C=less than 50 nM.

TABLE 16

| | Beta Arrestin |
|---|---|
| Compd. | EC₅₀ |
| IT001 | B |
| IT002 | B |
| IT003 | C |
| IT004 | C |
| IT005 | A |
| IT006 | B |
| IT007 | A |
| IT008 | A |
| IT009 | A |
| IT010 | A |
| IT011 | B |
| IT012 | A |
| IT013 | A |
| IT014 | C |
| IT015 | C |
| IT016 | C |
| IT017 | C |
| IT018 | A |
| IT019 | A |
| IT020 | A |
| IT021 | A |
| IT022 | A |
| IT023 | A |
| IT024 | A |
| IT025 | A |
| IT026 | A |
| IT027 | B |
| IT028 | C |
| IT029 | C |
| IT030 | A |
| IT031 | C |
| IT032 | C |
| IT033 | C |
| IT034 | C |
| IT035 | C |
| IT036 | A |
| IT037 | B |
| IT038 | B |
| IT039 | B |
| IT040 | A |
| IT041 | A |
| IT042 | C |
| IT043 | C |
| IT044 | C |
| IT045 | A |
| IT046 | C |
| IT047 | C |
| IT048 | C |
| IT049 | C |
| IT050 | C |
| IT051 | B |
| IT053 | A |
| IT054 | A |
| IT055 | B |
| IT056 | C |
| IT057 | A |
| IT058 | A |
| IT059 | B |
| IT060 | A |
| IT061 | C |
| IT062 | A |
| IT063 | A |
| IT064 | C |
| IT065 | B |
| IT066 | B |
| IT067 | B |
| IT068 | A |
| IT069 | C |
| IT070 | C |
| IT071 | C |
| IT072 | C |
| IT073 | B |
| IT074 | B |
| IT075 | C |
| IT076 | A |
| IT077 | B |
| IT078 | C |
| IT079 | A |
| IT080 | A |
| IT081 | A |
| IT082 | B |
| IT083 | A |
| IT084 | C |
| IT085 | C |
| IT086 | B |
| IT087 | C |
| IT088 | C |
| IT089 | A |
| IT090 | C |
| IT091 | B |
| IT092 | A |
| IT093 | A |
| IT094 | C |
| IT095 | C |
| IT096 | C |
| IT097 | B |
| IT098 | C |
| IT099 | B |
| IT100 | C |
| IT101 | A |
| IT102 | C |
| IT103 | C |
| IT104 | C |
| IT105 | B |
| IT106 | B |
| IT107 | C |
| IT108 | A |
| IT109 | C |
| IT110 | C |
| IT111 | C |
| IT112 | C |
| IT113 | A |
| IT114 | C |
| IT115 | B |
| IT116 | A |
| IT117 | C |
| IT118 | A |
| IT119 | C |
| IT120 | C |
| IT121 | C |
| IT122 | C |
| IT123 | C |
| IT124 | A |
| IT125 | A |
| IT126 | C |
| IT127 | A |
| IT128 | A |
| IT129 | B |
| IT130 | A |
| IT131 | B |
| IT132 | B |
| IT133 | B |
| IT134 | B |
| IT135 | B |
| IT136 | C |
| IT137 | B |
| IT138 | C |
| IT139 | B |
| IT140 | B |
| IT141 | A |
| IT142 | A |
| IT143 | A |
| IT144 | A |
| IT145 | C |
| IT147 | B |
| IT148 | A |
| IT149 | C |
| IT150 | B |
| IT151 | C |
| IT151A | C |
| IT151B | C |
| IT152 | A |
| IT153 | A |
| IT154 | A |
| IT155 | C |
| IT156 | B |
| IT157 | B |
| IT158 | B |
| IT159 | B |
| IT160 | B |
| IT161 | A |
| IT162 | A |
| IT163 | A |
| IT164 | C |
| IT165 | B |
| IT166 | B |
| IT167 | A |
| IT168 | A |
| IT169 | B |
| IT170 | B |
| IT171 | B |
| IT172 | C |
| IT173 | A |
| IT174 | A |
| IT175 | A |
| IT176 | A |
| IT177 | A |
| IT178 | B |
| IT179 | B |
| IT180 | A |
| IT181 | A |
| IT182 | A |
| IT183 | A |
| IT184 | A |
| IT185 | A |
| IT186 | A |
| IT187 | A |
| IT188 | B |
| IT189 | B |
| IT190 | A |
| IT191 | A |
| IT192 | A |
| IT193 | B |
| IT194 | A |
| IT195 | B |
| IT196 | C |
| IT197 | C |
| IT198 | C |
| IT199 | C |
| IT200 | A |
| IT201 | C |
| IT202 | B |
| IT203 | A |
| IT204 | A |
| IT205 | A |
| IT206 | A |
| IT207 | A |
| IT208 | A |
| IT209 | A |
| IT210 | A |
| IT211 | B |
| IT212 | B |
| IT213 | A |
| IT214 | B |
| IT215 | B |
| IT216 | A |
| IT217 | A |
| IT218 | A |
| IT219 | A |
| IT220 | B |
| IT221 | B |
| IT222 | B |
| IT223 | B |
| IT224 | C |
| IT225 | B |
| IT226 | C |
| IT227 | C |
| IT228 | C |
| IT229 | C |
| IT230 | C |
| IT231 | B |
| IT232 | C |
| IT234 | C |
| IT235 | C |
| IT236 | C |
| IT237 | B |
| IT238 | B |
| IT239 | A |
| IT240 | A |
| IT241 | A |
| IT242 | A |
| IT243 | A |
| IT244 | C |
| IT245 | C |
| IT246 | C |
| IT247 | B |
| IT248 | A |
| IT249 | B |
| IT250 | B |
| IT251 | B |
| IT252 | A |
| IT253 | B |
| IT254 | B |
| IT255 | C |
| IT256 | C |
| IT257 | C |
| IT258 | C |
| IT259 | B |
| IT260 | C |
| IT261 | C |
| IT262 | C |
| IT263 | C |
| IT264 | B |
| IT265 | B |
| IT266 | C |
| IT267 | B |
| IT268 | B |
| IT269 | B |
| IT270 | C |
| IT271 | B |
| IT272 | B |
| IT273 | B |
| IT274 | B |
| IT275 | B |
| IT276 | A |
| IT277 | B |
| IT278 | B |
| IT279 | C |
| IT280 | C |
| IT281 | C |
| IT282 | C |
| IT283 | A |
| IT284 | B |
| IT285 | C |
| IT286 | B |
| IT287 | A |
| IT288 | B |
| IT289 | A |
| IT290 | A |
| IT291 | C |
| IT292 | A |
| IT293 | B |
| IT294 | B |
| IT295 | C |
| IT296 | C |
| IT297 | C |
| IT298 | B |
| IT299 | B |
| IT300 | C |
| IT301 | C |
| IT303 | B |
| IT304 | C |
| IT305 | C |
| IT306 | C |
| IT307 | C |
| IT308 | C |
| IT309 | C |
| IT310 | B |
| IT311 | B |
| IT312 | C |
| IT313 | B |
| IT314 | C |
| IT315 | C |
| IT316 | C |
| IT317 | A |
| IT318 | A |
| IT319 | A |
| IT320 | A |
| IT321 | B |
| IT322 | A |
| IT323 | B |
| IT324 | B |
| IT325 | B |
| IT326 | B |
| IT327 | B |
| IT328 | B |
| IT329 | B |
| IT330 | B |
| IT331 | B |
| IT332 | B |
| IT333 | B |
| IT334 | A |
| IT335 | A |
| IT336 | C |
| IT337 | A |
| IT338 | B |
| IT339 | B |
| IT340 | B |
| IT341 | C |
| IT342 | C |
| IT343 | C |
| IT344 | C |
| IT345 | C |
| IT346 | C |
| IT347 | C |
| IT348 | C |
| IT349 | A |
| IT350 | A |
| IT351 | A |
| IT352 | A |
| IT353 | B |
| IT354 | A |
| IT355 | C |
| IT356 | B |
| IT357 | B |
| IT358 | B |
| IT359 | B |
| IT360 | A |
| IT361 | C |
| IT362 | A |
| IT363 | A |
| IT364 | C |
| IT365 | C |
| IT366 | C |
| IT367 | C |
| IT368 | A |
| IT369 | B |
| IT370 | A |
| IT371 | A |
| IT372 | A |
| IT373 | A |
| IT374 | C |
| IT375 | C |
| IT376 | A |
| IT377 | A |
| IT378 | A |
| IT379 | B |
| IT380 | B |
| IT381 | B |
| IT382 | A |
| IT383 | A |
| IT384 | A |
| IT385 | B |
| IT386 | B |
| IT387 | B |
| IT388 | B |
| IT389 | C |
| IT390 | C |
| IT391 | B |
| IT392 | B |
| IT393 | C |
| IT394 | C |
| IT395 | A |
| IT396 | B |
| IT397 | A |
| IT398 | A |
| IT399 | A |
| IT400 | A |
| IT401 | A |
| IT402 | A |
| IT403 | A |
| IT404 | A |
| IT405 | A |
| IT406 | C |
| IT407 | C |
| IT408 | C |
| IT409 | B |
| IT410 | A |
| IT411 | B |
| IT412 | A |
| IT413 | A |
| IT414 | A |
| IT415 | A |
| IT416 | A |
| IT417 | B |
| IT418 | C |
| IT419 | B |
| IT420 | C |
| IT421 | A |
| IT422 | A |
| IT423 | C |
| IT424 | C |
| IT425 | C |
| IT426 | B |
| IT427 | A |
| IT428 | C |
| IT429 | A |
| IT430 | A |
| IT431 | A |
| IT432 | A |
| IT433 | A |
| IT434 | C |
| IT435 | C |
| IT436 | C |
| IT437 | C |
| IT438 | C |
| IT439 | C |
| IT440 | B |
| IT441 | A |
| IT442 | A |
| IT443 | A |
| IT444 | C |
| IT445 | A |
| IT446 | C |
| IT447 | C |
| IT448 | C |
| IT449 | C |
| IT450 | C |
| IT451 | C |
| IT452 | C |
| IT453 | C |
| IT454 | C |
| IT455 | C |
| IT456 | C |
| IT457 | B |
| IT458 | A |
| IT459 | C |
| IT460 | C |
| IT461 | C |
| IT462 | C |
| IT463 | C |
| IT464 | C |
| IT465 | C |
| IT466 | A |
| IT467 | C |
| IT468 | C |
| IT469 | C |
| IT470 | C |
| IT471 | B |
| IT472 | A |
| IT473 | C |
| IT474 | C |
| IT476 | C |
| IT477 | C |
| IT478 | B |
| IT479 | C |
| IT480 | A |
| IT481 | C |
| IT482 | C |
| IT483 | C |
| IT484 | C |
| IT485 | C |
| IT486 | B |
| IT488 | B |
| IT489 | C |
| IT491 | B |
| IT492 | B |
| IT493 | B |
| IT494 | C |
| IT495 | C |
| IT496 | C |
| IT498 | C |
| IT499 | A |
| IT500 | C |
| IT501 | B |
| IT502 | B |
| IT503 | B |
| IT504 | B |
| IT505 | B |
| IT506 | C |
| IT507 | B |
| IT508 | B |
| IT509 | B |
| IT510 | B |
| IT511 | C |
| IT512 | B |
| IT513 | C |
| IT514 | C |

TABLE 17

| | Cell Migration |
|---|---|
| Compd. | EC₅₀ |
| IT001 | B |
| IT002 | B |
| IT003 | B |
| IT004 | C |
| IT005 | A |
| IT006 | B |
| IT007 | A |
| IT008 | A |
| IT009 | A |
| IT010 | A |
| IT011 | A |
| IT012 | A |
| IT013 | A |
| IT014 | B |
| IT015 | B |
| IT016 | B |
| IT017 | C |
| IT018 | A |
| IT019 | A |
| IT020 | A |
| IT021 | A |
| IT022 | A |
| IT023 | A |
| IT024 | A |
| IT025 | A |
| IT026 | A |
| IT027 | A |
| IT028 | C |
| IT029 | B |
| IT030 | A |
| IT031 | B |
| IT032 | B |
| IT033 | C |
| IT034 | B |
| IT035 | B |
| IT036 | A |
| IT038 | A |
| IT039 | A |
| IT040 | A |
| IT041 | A |
| IT042 | B |
| IT043 | B |
| IT044 | B |
| IT045 | A |
| IT046 | B |
| IT047 | C |
| IT048 | B |
| IT049 | B |
| IT050 | B |
| IT051 | B |
| IT052 | A |
| IT053 | A |
| IT054 | A |
| IT055 | B |
| IT056 | B |
| IT057 | A |
| IT058 | A |
| IT059 | A |
| IT060 | A |
| IT061 | B |
| IT062 | A |
| IT063 | A |
| IT064 | B |
| IT065 | A |
| IT066 | A |
| IT067 | A |
| IT068 | A |
| IT069 | B |
| IT070 | C |
| IT071 | B |
| IT072 | A |
| IT073 | B |
| IT074 | A |
| IT075 | B |
| IT078 | B |
| IT079 | A |
| IT081 | A |
| IT082 | B |
| IT083 | A |
| IT084 | B |
| IT085 | A |
| IT086 | A |
| IT087 | C |
| IT088 | B |
| IT089 | A |
| IT090 | B |
| IT091 | A |
| IT092 | A |
| IT093 | A |
| IT094 | B |
| IT095 | C |
| IT096 | B |
| IT097 | A |
| IT098 | C |
| IT099 | A |
| IT101 | A |
| IT102 | B |
| IT103 | B |
| IT104 | B |
| IT105 | B |
| IT106 | A |
| IT107 | B |
| IT108 | A |
| IT110 | B |
| IT111 | B |
| IT112 | B |
| IT114 | C |
| IT115 | A |
| IT117 | B |
| IT118 | A |
| IT119 | B |
| IT120 | B |
| IT121 | C |
| IT122 | B |
| IT123 | B |
| IT124 | B |
| IT125 | B |
| IT126 | B |
| IT128 | A |
| IT129 | A |
| IT130 | A |
| IT131 | A |
| IT132 | A |
| IT133 | A |
| IT134 | A |
| IT135 | A |
| IT136 | B |
| IT137 | B |
| IT138 | B |
| IT139 | B |
| IT140 | A |
| IT141 | A |
| IT142 | A |
| IT145 | A |
| IT146 | B |
| IT147 | A |
| IT148 | A |
| IT149 | B |
| IT150 | B |
| IT151 | B |
| IT151A | C |
| IT151B | B |
| IT153 | A |
| IT154 | A |
| IT155 | C |
| IT156 | A |
| IT157 | A |
| IT158 | B |
| IT159 | A |
| IT160 | B |
| IT162 | A |
| IT164 | A |
| IT165 | A |
| IT166 | A |
| IT167 | A |
| IT168 | A |
| IT169 | A |
| IT170 | A |
| IT171 | A |
| IT172 | C |
| IT173 | A |
| IT174 | A |
| IT178 | A |
| IT179 | A |
| IT180 | A |
| IT181 | A |
| IT182 | A |
| IT183 | A |
| IT184 | A |
| IT185 | A |
| IT186 | A |
| IT187 | A |
| IT188 | A |
| IT189 | A |
| IT190 | A |
| IT191 | A |
| IT192 | A |
| IT193 | A |
| IT194 | A |
| IT195 | A |
| IT196 | B |
| IT197 | C |
| IT198 | B |
| IT199 | C |
| IT201 | B |
| IT224 | C |
| IT226 | C |
| IT227 | C |
| IT228 | B |
| IT229 | B |
| IT230 | B |
| IT231 | B |
| IT232 | B |
| IT234 | C |
| IT235 | C |
| IT236 | B |
| IT245 | B |
| IT246 | B |
| IT255 | B |
| IT257 | B |
| IT258 | C |
| IT259 | A |
| IT260 | B |
| IT261 | B |
| IT262 | B |
| IT263 | B |
| IT266 | B |
| IT270 | B |
| IT279 | B |
| IT281 | B |
| IT282 | C |
| IT284 | B |
| IT285 | B |
| IT291 | B |
| IT295 | B |
| IT296 | B |
| IT300 | C |
| IT301 | B |
| IT303 | B |
| IT304 | B |
| IT305 | C |
| IT306 | C |
| IT307 | B |
| IT308 | C |
| IT309 | C |
| IT310 | A |
| IT311 | B |
| IT312 | B |
| IT313 | A |
| IT314 | A |
| IT315 | C |
| IT316 | C |
| IT336 | B |
| IT341 | B |
| IT344 | B |
| IT345 | B |
| IT347 | B |
| IT348 | B |
| IT355 | C |
| IT364 | B |
| IT365 | B |
| IT374 | B |
| IT375 | B |
| IT389 | B |
| IT390 | B |
| IT394 | B |
| IT401 | B |
| IT406 | B |
| IT407 | C |
| IT408 | C |
| IT417 | B |
| IT418 | B |
| IT420 | B |
| IT423 | B |
| IT424 | C |
| IT425 | C |
| IT428 | B |
| IT435 | C |
| IT436 | B |
| IT437 | B |
| IT438 | B |
| IT439 | A |
| IT440 | A |
| IT446 | C |
| IT447 | C |
| IT448 | B |
| IT449 | C |
| IT450 | C |
| IT451 | C |
| IT452 | C |
| IT453 | C |
| IT454 | B |
| IT455 | C |
| IT456 | B |
| IT457 | A |
| IT459 | B |
| IT460 | C |
| IT461 | B |
| IT462 | C |
| IT463 | C |
| IT464 | B |
| IT465 | C |
| IT467 | B |
| IT468 | C |
| IT469 | B |
| IT474 | C |
| IT476 | A |
| IT477 | B |
| IT478 | B |
| IT479 | A |
| IT481 | C |
| IT482 | B |
| IT483 | B |
| IT484 | B |
| IT485 | C |
| IT486 | A |
| IT488 | B |
| IT489 | C |
| IT491 | A |
| IT492 | A |
| IT493 | A |
| IT494 | B |
| IT495 | B |
| IT496 | C |
| IT498 | B |
| IT499 | A |
| IT500 | B |
| IT502 | A |
| IT503 | A |
| IT505 | B |
| IT506 | B |
| IT507 | A |
| IT509 | A |
| IT510 | A |
| IT511 | B |
| IT512 | A |
| IT513 | B |

TABLE 18

| | Ca Flux LPA1 |
|---|---|
| Compd. | IC₅₀ |
| IT003 | C |
| IT004 | C |
| IT009 | A |
| IT010 | A |
| IT014 | C |
| IT015 | B |
| IT016 | C |
| IT017 | C |
| IT018 | A |
| IT020 | A |
| IT021 | A |
| IT022 | A |
| IT023 | A |
| IT026 | A |
| IT027 | B |
| IT028 | C |
| IT029 | B |
| IT030 | A |
| IT031 | B |
| IT032 | B |
| IT033 | B |
| IT034 | B |
| IT035 | A |
| IT040 | A |
| IT041 | B |
| IT043 | B |
| IT046 | A |
| IT047 | B |
| IT048 | C |
| IT050 | B |
| IT051 | B |
| IT056 | B |
| IT061 | C |
| IT064 | C |
| IT066 | B |
| IT067 | B |
| IT069 | C |
| IT070 | C |
| IT071 | B |
| IT072 | B |
| IT073 | C |
| IT078 | B |
| IT091 | B |
| IT095 | C |
| IT098 | C |
| IT099 | A |
| IT102 | C |
| IT103 | C |
| IT104 | C |
| IT105 | B |
| IT106 | C |
| IT107 | C |
| IT108 | A |
| IT111 | B |
| IT112 | C |
| IT114 | B |
| IT117 | C |
| IT119 | A |
| IT120 | A |
| IT121 | A |
| IT122 | A |
| IT123 | B |
| IT124 | A |
| IT125 | C |
| IT126 | C |
| IT136 | B |
| IT151A | B |
| IT155 | B |
| IT172 | B |
| IT196 | C |
| IT197 | C |
| IT198 | C |
| IT199 | C |
| IT224 | C |
| IT226 | C |
| IT227 | C |
| IT228 | B |
| IT229 | B |
| IT234 | C |
| IT235 | B |
| IT236 | C |
| IT245 | B |
| IT255 | B |
| IT258 | C |
| IT261 | A |
| IT262 | B |
| IT282 | B |
| IT300 | C |
| IT301 | C |
| IT303 | C |
| IT304 | C |
| IT305 | C |
| IT306 | C |
| IT307 | C |
| IT308 | B |
| IT309 | C |
| IT315 | C |
| IT316 | B |
| IT341 | B |
| IT345 | C |
| IT374 | B |
| IT375 | B |
| IT407 | C |
| IT408 | C |
| IT423 | C |
| IT424 | C |
| IT425 | C |
| IT434 | B |
| IT435 | C |
| IT436 | C |
| IT438 | B |
| IT446 | C |
| IT447 | C |
| IT448 | B |
| IT449 | A |
| IT450 | B |
| IT451 | B |
| IT452 | C |
| IT453 | B |
| IT474 | C |
| IT479 | B |
| IT481 | A |
| IT483 | B |
| IT484 | C |
| IT485 | B |
| IT489 | B |
| IT494 | C |
| IT495 | A |
| IT496 | A |

### Clinical Trials in Humans

Clinical trials can be run in multiple conditions. The details of these trials differ based on the indication. Examples of clinical trials for assessment of clinical effect in idiopathic pulmonary fibrosis are provided below.

Although a duration of 72 weeks is specified in the examples below, other durations can also be employed, e.g., 52 weeks.

Clinical Trial in Humans with Idiopathic Pulmonary Fibrosis (IPF) Purpose

### Example #1

The efficacy of treatment with a compound of a preferred embodiment compared with placebo in patients with idiopathic pulmonary fibrosis (IPF) and the safety of treatment with a compound of a preferred embodiments compared with placebo in patients with IPF is assessed.

The primary outcome variable is the absolute change in percent predicted forced vital capacity (FVC) from baseline to Week 72. Other possible end-points would include, but are not limited to: mortality, progression free survival, change in rate of FVC decline, change in Sp02, and change in biomarkers (HRCT image analysis; molecular and cellular markers of disease activity). Secondary outcome measures include: composite outcomes of important IPF-related events; progression-free survival; categorical assessment of absolute change in percent predicted FVC from baseline to Week 72; change in Shortness-of-Breath from baseline to Week 72; change in percent predicted hemoglobin (Hb)-corrected carbon monoxide diffusing capacity (DLco) of the lungs from baseline to Week 72; change in oxygen saturation during the 6 minute walk test (6MWT) from baseline to Week 72; change in high-resolution computed tomography (HRCT) assessment from baseline to Week 72; change in distance walked in the 6MWT from baseline to Week 72.

Patients eligible for this study include, but are not limited to: those patients that satisfy the following inclusion criteria: diagnosis of IPF; 40 to 80 years of age; FVC≧50% predicted value; DLco≧35% predicted value; either FVC or DLco≧90% predicted value; no improvement in past year; able to walk 150 meters in 6 minutes and maintain saturation≧83% while on no more than 6 L/min supplemental oxygen.

Patients are excluded from this study if they satisfy any of the following criteria: unable to undergo pulmonary function testing; evidence of significant obstructive lung disease or airway hyper-responsiveness; in the clinical opinion of the investigator, the patient is expected to need and be eligible for a lung transplant within 72 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials.

Patients are orally dosed with either placebo or an amount of a compound of a preferred embodiment (1 mg/day-1000 mg/day). The primary outcome variable will be the absolute change in percent predicted FVC from Baseline to Week 72. Patients will receive blinded study treatment from the time of randomization until the last patient randomized has been treated for 72 weeks. A Data Monitoring Committee (DMC) will periodically review safety and efficacy data to ensure patient safety.

After week 72, patients who meet the Progression of Disease (POD) definition, which is a ≧10% absolute decrease in percent predicted FVC or a ≧15% absolute decrease in percent predicted DLco, will be eligible to receive permitted IPF therapies in addition to their blinded study drug. Permitted IPF therapies include, but are not limited to: corticosteroids, azathioprine, cyclophosphamide, and N-acetyl-cysteine.

In a preferred aspect, a method is provided of administering an LPA1 antagonist of a preferred embodiment to a patient with pulmonary fibrosis (e.g., a patient with IPF), wherein said patient is selected, or diagnosed, or identified to have one or more of the following criteria: (1) ratio of forced expiratory volume in one second (FEV1) to forced vital capacity volume (FVC), or FEV1/FVC, is greater than 0.80, (2) percent of predicted FVC (% FVC) is 90% or less, for example ranging from 50% to 90%, inclusive of both endpoints, and (3) time since diagnosis of IPF is at least six months and up to 48 months. The terms “selecting,” “diagnosing” and “identifying” are used synonymously with respect to a patient.

Clinical Trial in Humans with Idiopathic Pulmonary Fibrosis (IPF) Purpose

### Example #2

The efficacy of treatment with a compound of a preferred embodiment compared with placebo in patients with idiopathic pulmonary fibrosis (IPF) and the safety of treatment with a compound of a preferred embodiments compared with placebo in patients with IPF is assessed.

The primary outcome variable includes, but is not limited to, the absolute change in percent predicted forced vital capacity (FVC) from baseline to Week 72. Secondary outcome measures include, but are not limited to: composite outcomes of important IPF-related events; progression-free survival; categorical assessment of absolute change in percent predicted FVC from baseline to Week 72; change in Shortness-of-Breath from baseline to Week 72; change in percent predicted hemoglobin (Hb)-corrected carbon monoxide diffusing capacity (DLco) of the lungs from baseline to Week 72; change in oxygen saturation during the 6 minute walk test (6MWT) from baseline to Week 72; change in high-resolution computed tomography (HRCT) assessment from baseline to Week 72; change in distance walked in the 6MWT from baseline to Week 72.

Patients eligible for this study include, but are not limited to, those patients that satisfy the following inclusion criteria: diagnosis of IPF; 40 to 80 years of age; FVC≧50% predicted value; DLco≧35% predicted value; either FVC or DLco≧90% predicted value; no improvement in past year; able to walk 150 meters in 6 minutes and maintain saturation≧83% while on no more than 6 L/min supplemental oxygen.

Patients are excluded from this study if they satisfy any of the following criteria, including but not limited to: unable to undergo pulmonary function testing; evidence of significant obstructive lung disease or airway hyper-responsiveness; in the clinical opinion of the investigator, the patient is expected to need and be eligible for a lung transplant within 72 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials.

Patients are orally dosed with either placebo or an amount of a compound of a preferred embodiment (1 mg/day-1000 mg/day or more). The primary outcome variable includes, but is not limited to, the absolute change in percent predicted FVC from Baseline to Week 72. Patients receive blinded study treatment from the time of randomization until the last patient randomized has been treated for 72 weeks. A Data Monitoring Committee (DMC) periodically reviews safety and efficacy data to ensure patient safety.

After week 72, patients who meet the Progression of Disease (POD) definition, which is a ≧10% absolute decrease in percent predicted FVC or a ≧15% absolute decrease in percent predicted DLco, are eligible to receive permitted IPF therapies in addition to their blinded study drug. Permitted IPF therapies include, but are not limited to, corticosteroids, azathioprine, cyclophosphamide, and N-acetyl-cysteine.

### Treatment of Ideopathic Pulmonary Fibrosis

A compound of a preferred embodiment can be administered to a patient in need of therapy, and can be used in methods of preparing or packaging medicaments, containers, packages, and kits comprising the compound of a preferred embodiment. The patient may have pulmonary fibrosis, such as IPF, and the medicament can be used for treatment of pulmonary fibrosis, or IPF. A selected group of IPF patients that are more likely to experience FVC decline and disease progression over a period of a year can be identified and treated. Their greater rate of progression, as reflected by a greater rate of decrease in respiratory parameters such as FVC, correlates with a greater relative magnitude of treatment effect. In certain embodiments, IPF patients with the following criteria experience a greater FVC decline, as measured by % FVC change from baseline or proportion of patients with 10% or greater % FVC decline at a specified timepoint, compared to patients that do not meet the criteria. Patients with the following criteria also exhibit a greater observed treatment effect on alleviating the extent of FVC decline compared to patients that do not meet the criteria: (a) % FVC 50%-90%; (b) FEV1/FVC ratio>0.80; (c) Time since IPF diagnosis >0.5 years and <48 months;

A method of treating pulmonary fibrosis, optionally IPF, is provided comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of the compound of a preferred embodiment.

In a related aspect, use is provided of the compound of a preferred embodiment in treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.

In a further related aspect, the compound of a preferred embodiment is used in preparation of a medicament for treating pulmonary fibrosis in a patient that exhibits (i) percent of predicted forced vital capacity volume (% FVC) of about 90% or less or (ii) ratio of forced expiratory volume in one second (FEY1) to forced vital capacity volume (FVC) of about 0.80 or greater, or both.

Optionally, in some or any of these embodiments, % FVC ranges from about 50% to about 90%. In some or any embodiments, the patient has been diagnosed with pulmonary fibrosis, optionally IPF, for at least six months, and optionally less than 48 months. In some or any embodiments, optionally the patient is also selected to exhibit a percent of diffusing capacity (% DL_{co}) of about 90% or less, for example, ranging from 30% to 90%, or 30% to 60%, inclusive of both endpoints. In some or any embodiments, the FEV1/FVC ratio is greater than 0.9. In some or any embodiments, the % FVC is less than 80%, 70%, or 60%. In some or any embodiments, the % DL_{co} is less than 90%, 80%, 70%, 60%, or 50%, or less than 40%. In most cases the patient is diagnosed with IPF through a High Resolution Computed Tomography (HRCT) scan, optionally with confirmation through surgical lung biopsy.

In any of the aspects or embodiments, the therapeutically effective amount of the compound of a preferred embodiment being administered may be a total daily dosage of from 1-4000 mg per day or more, e.g., at least about 1800 mg per day, or about 2400 mg or about 2403 mg per day, optionally administered in divided doses three times per day, with food. In any of the aspects of embodiments, the total daily dosage may be about 1200 to about 4000 mg per day, or about 1600 to about 3600 mg per day. In any of the aspects of the invention, the daily dosage may be administered in divided doses three times a day, or two times a day, or alternatively is administered in a single dose once a day. In any of the aspects of the invention, the compound of a preferred embodiment may be administered with food. For example, the daily dosage of 2400 mg or 2403 mg the compound of a preferred embodiment per day may be administered as follows: 801 mg taken three times a day, with food.

The compound of a preferred embodiment can be dosed at a total amount of from 1-4000 mg per day or more, or from about 50 to about 2400 mg per day. The dosage can be divided into two or three doses over the day. Specific amounts of the total daily amount of the therapeutic contemplated for the disclosed methods include about 50 mg, about 100 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg, about 1050 mg, about 1068 mg, about 1100 mg, about 1150 mg, about 1200 mg, about 1250 mg, about 1300 mg, about 1335 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1869 mg, about 1900 mg, about 1950 mg, about 2000 mg, about 2050 mg, about 2100 mg, about 2136 mg, about 2150 mg, about 2200 mg, about 2250 mg, about 2300 mg, about 2350 mg, and about 2400 mg.

Dosages of the compound of preferred embodiments can alternately be administered as a dose measured in mg/kg. Contemplated mg/kg doses of the disclosed therapeutics include, e.g., about 1 mg/kg to about 40 mg/kg. Specific ranges of doses in mg/kg include about 20 mg/kg to about 40 mg/kg, or about 30 mg/kg to about 40 mg/kg.

In another aspect, a package or kit is provided comprising the compound of a preferred embodiment, optionally in a container, and a package insert, package label, instructions, or other labeling including any of the criteria for patient selection described herein. The package insert, package label, instructions or other labeling may further comprise directions for treating IPF by administering the compound of a preferred embodiment, e.g., at a dosage of at least about 1800 mg per day, or a dosage of about 2400 mg or about 2403 mg per day.

In related aspect, a method of preparing or packaging a medicament comprising the compound of a preferred embodiment, optionally in a container, together with a package insert or package label or instructions including any of the foregoing information or recommendations.

In some embodiments, a method of treating IPF is disclosed comprising providing, selling, or delivering any of the kits of disclosed herein to a hospital, physician, or patient.

The following patent publications include disclosures relating to diseases, disorders, or conditions that may be associated with one or more of the lysophosphatidic acid receptors, the contents of which relating to said diseases, disorders, or conditions are hereby incorporated by reference herein: PCT Intl. Publ. No. WO/2011017350-A1; PCT Intl. Publ. No. WO/2010141768-A1; PCT Intl. Publ. No. WO/2010077883-A1; PCT Intl. Publ. No. WO/2010077882-A1; PCT Intl. Publ. No. WO/2010068775-A1; U.S. Pat. Publ. No. US-20110098352-A1; U.S. Pat. Publ. No. US-20110098302-A1; U.S. Pat. Publ. No. US-20110082181-Aa; U.S. Pat. Publ. No. US-20110082164-A1; U.S. Pat. Publ. No. US-20100311799-A1; U.S. Pat. Publ. No. US-20100152257-A1; PCT Intl. Publ. No. WO/2010141761-A1; PCT Intl. Publ. No. WO/2011041729-A1; PCT Intl. Publ. No. WO/2011041694-A1; PCT Intl. Publ. No. WO/2011041462-A1; and PCT Intl. Publ. No. WO/2011041461-A1.

### Pharmaceutical Compositions

Parenteral Pharmaceutical Composition

To prepare a parenteral pharmaceutical composition suitable for administration by injection (subcutaneous, intravenous, or the like), 100 mg of a water-soluble salt/soluble material itself/solubilized complex of a compound of a preferred embodiment is dissolved in sterile water and then mixed with 10 mL of 0.9% sterile saline. The mixture is incorporated into a dosage unit form suitable for administration by injection.

Injectable Pharmaceutical Composition

To prepare an injectable formulation, 1.2 g of a compound of Formulas (I), 2.0 mL of sodium acetate buffer solution (0.4 M), HCl (1 N) or NaOH (1 M) (q.s. to suitable pH), water (distilled, sterile) (q.s. to 20 mL) are mixed. All of the above ingredients, except water, are combined and stirred and if necessary, with slight heating if necessary. A sufficient quantity of water is then added.

Oral Pharmaceutical Composition

To prepare a pharmaceutical composition for oral delivery, 100 mg of a compound of a preferred embodiment is mixed with 750 mg of starch. The mixture is incorporated into an oral dosage unit, such as a hard gelatin capsule, or 100 mg of compound is granulated with binder solution such as starch solution along with suitable diluents such as microcrystalline cellulose or like, disintegrants such as cross caramellose sodium, dry the resultant mixture and add lubricant and compress into tablet which is suitable for oral administration.

Sublingual (Hard Lozenge) Pharmaceutical Composition

To prepare a pharmaceutical composition for buccal delivery, such as a hard lozenge, 100 mg of a compound of a preferred embodiment is mixed with 420 mg of powdered sugar/mannitol/xylitol or such sugars that provide negative heat of solution to the system, 1.6 mL of light corn syrup, 2.4 mL distilled water, and 0.42 mL mint extract or other flavorants. The mixture is blended and poured into a mold to form a lozenge suitable for buccal administration.

Fast-Disintegrating Sublingual Tablet

A fast-disintegrating sublingual tablet is prepared by mixing 48.5% by weigh of a compound of a preferred embodiment, 20% by weight of microcrystalline cellulose (KG-802), 24.5% by weight of either mannitol or modified dextrose or combination that help dissolve the compressed tablet faster in the mouth, 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of the compound of a preferred embodiment with the total quantity of microcrystalline cellulose (MCC) and mannitol/modified dextrose or combination, and two-thirds of the quantity of low-substituted hydroxypropyl cellulose (L-HPC) by using a three dimensional manual mixer (Inversina®, Bioengineering AG, Switzerland) for 4.5 minutes. All of the magnesium stearate (MS) and the remaining one-third of the quantity of L-HPC are added 30 seconds before the end of mixing.

Inhalation Pharmaceutical Composition

To prepare a pharmaceutical composition for inhalation delivery, 20 mg of a compound of a preferred embodiment is mixed with 50 mg of anhydrous citric acid and 100 mL of 0.9% sodium chloride solution. The mixture is incorporated into an inhalation delivery unit, such as a nebulizer, which is suitable for inhalation administration.

Nebulizer Suspension Pharmaceutical Composition

In another embodiment, a compound of a preferred embodiment (500 mg) is suspended in sterile water (100 mL); Span 85 (1 g) is added followed by addition of dextrose (5.5 g) and ascorbic acid (10 mg). Benzalkonium chloride (3 mL of a 1:750 aqueous solution) is added and the pH is adjusted to 7 with phosphate buffer. The suspension is packaged in sterile nebulizers.

Rectal Gel Pharmaceutical Composition

To prepare a pharmaceutical composition for rectal delivery, 100 mg of a compound of a preferred embodiment is mixed with 2.5 g of methylcellulose (1500 mPa), 100 mg of methylparaben, 5 g of glycerin and 100 mL, of purified water. The resulting gel mixture is then incorporated into rectal delivery units, such as syringes, which are suitable for rectal administration.

Topical Gel Pharmaceutical Composition

To prepare a pharmaceutical topical gel composition, 100 mg of a compound of a preferred embodiment is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

Ophthalmic Solution

To prepare a pharmaceutical ophthalmic solution composition, 100 mg of a compound of a preferred embodiment is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

Nasal Spray Solution

To prepare a pharmaceutical nasal spray solution, 10 g of a compound of a preferred embodiment is mixed with 30 mL of a 0.05M phosphate buffer solution (pH 4.4). The solution is placed in a nasal administrator designed to deliver 100 μl of spray for each application.

While the disclosure has been illustrated and described in detail in the foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1.-32. (canceled)

33. A compound of Formula (II): 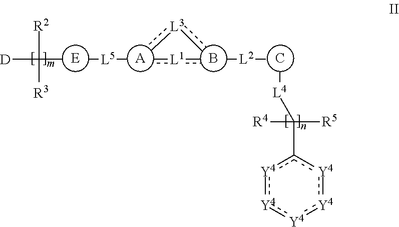
or a pharmaceutically acceptable salt thereof, wherein:
A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;
B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;
C is a ring system selected from the group consisting of 6-11 membered aryl, 5-11 membered heteroaryl, 5-11 membered heterocyclyl, and 5-11 membered carbocyclyl, wherein C is optionally substituted;
D is selected from —OH, 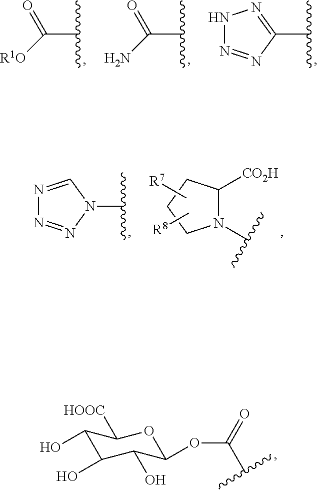
—NR¹³SOₚR¹⁴, —C(O)—NR¹³SOₚR¹⁴, 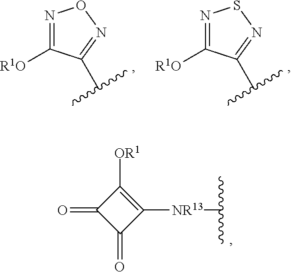
—SOₚR¹⁵, —SOₚNR¹⁶R¹⁷, or carboxylic acid isosteres;
E is absent or selected from 6-10 membered arylene, 3-11 membered carbocyclyl, 3-11 membered heterocyclyl or 5 to 10 membered heteroarylene, wherein E is optionally substituted;
L⁴ is selected from 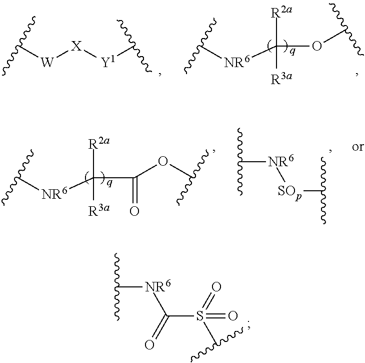
or alternatively, 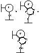
wherein 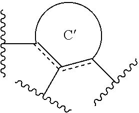
is selected from: 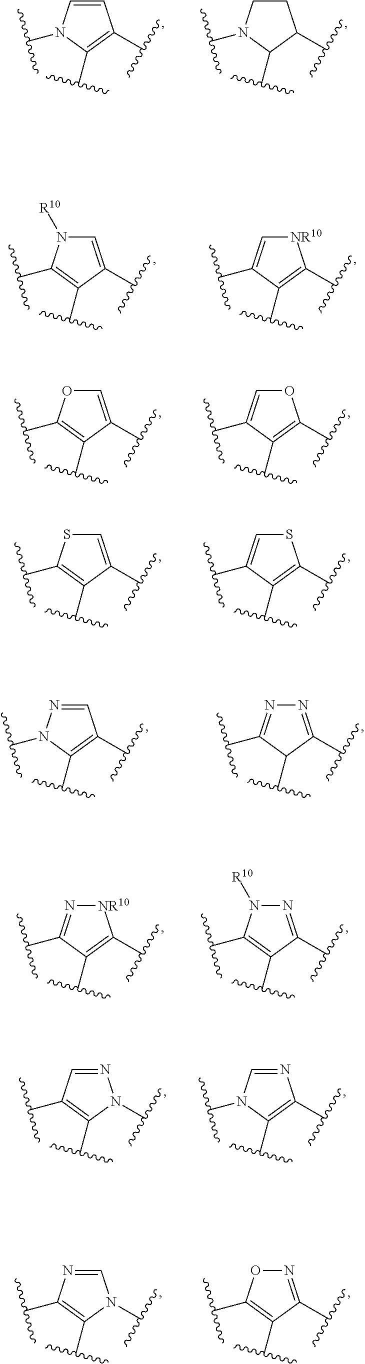 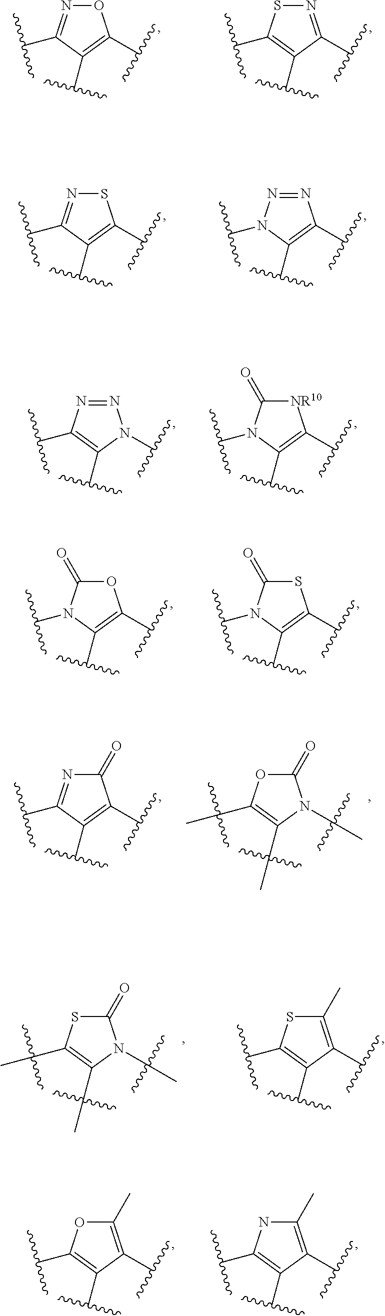 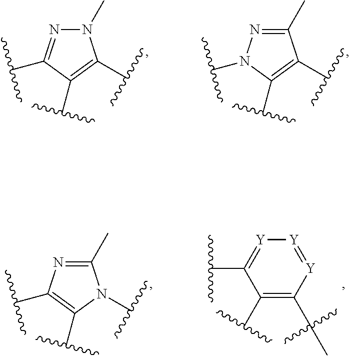
or optionally substituted variants thereof;
L¹ is selected from a single bond, a —CH₂— linker, 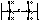
a —C≡C— linker, a —CH═CH— linker, or a ═C(R¹¹)— linker;
L² is selected from a single bond, a —CH₂— linker, 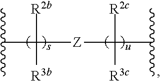
a —C≡C— linker, or a —CH═CH— linker;
L³ is absent or selected from 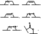
or a ═C(R¹¹)— linker;
L⁵ is selected from 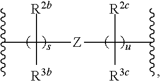
or a —C≡C— linker;
W is selected from C(R⁶)₂, NR⁶, or O;
X is selected from —C(O) or S(O)ₚ;
each Y is independently selected from CR⁶ or N;
Y¹ is selected from C(R⁶)₂, NR⁶, or O;
each Y⁴ is independently absent, CR⁹, C(R⁹)₂, N, or NH, provided that only one Y⁴ can be absent;
each Z is independently selected from C(O), O, S, S(O)₂, NR^{6a}, C(O)NR^{6b}, or S(O)₂NR^{6c};
R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;
R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;
or R² and R³ are joined together with the atom to which they are attached to form an optionally substituted cycloalkyl or an optionally substituted heterocyclyl;
or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;
each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;
each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;
each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;
each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;
each R¹¹ is independently selected from hydrogen, alkyl optionally substituted with one or more substituents selected from the group consisting of halogen, hydroxy and alkoxy; halogen; haloalkyl; or cyano;
each R¹³ and R¹⁴ is independently selected from hydrogen, alkyl, haloalkyl, aryl, or C₃₋₆ cycloalkyl;
R¹⁵ is selected from hydrogen, alkyl, haloalkyl, (carbocyclyl)alkyl, aryl, or C₃₋₆ cycloalkyl;
each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;
each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3ab}, or R^{2c} and R^{3c} are independently joined together with the atom to which they are attached to form an optionally substituted cycloalkyl or an optionally substituted heterocyclyl;
m is independently an integer from 0-3;
n is an integer from 0-3;
k is an integer from 0-3;
p is an integer from 1-2;
q is an integer from 1-6;
each s and u is independently an integer from 0 to 6; and [symbol]
represents a single or double bond, provided that
when A is 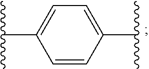 [SMILES: *c1ccc(*)cc1]
B is 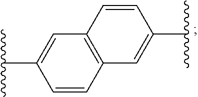 [SMILES: *c1ccc2cc(*)ccc2c1]
D is —C(O)OH; m is 0; E is absent;
L⁵ is —CH₂SCH₂CH₂—; L¹ is a single bond; L² is a single bond; and 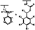
wherein R⁹ is selected from H or halogen and R⁴ is methyl; then C is not

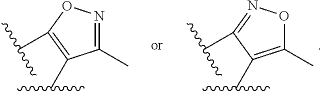

34. (canceled)

35. The compound or pharmaceutically acceptable salt thereof of claim 33, wherein
A is an acetylene or selected from the group consisting of 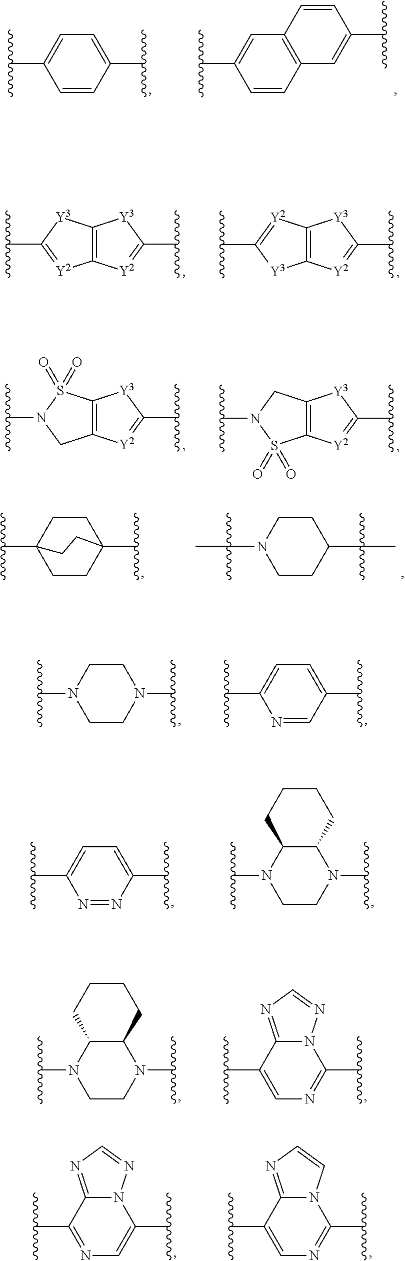 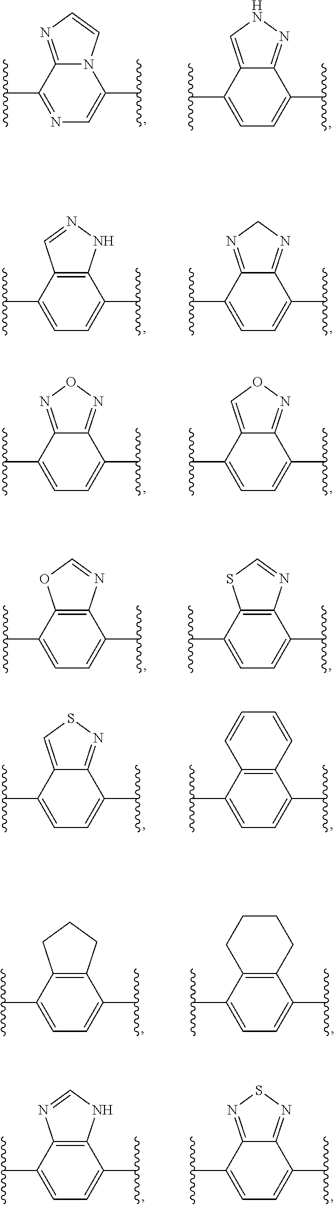 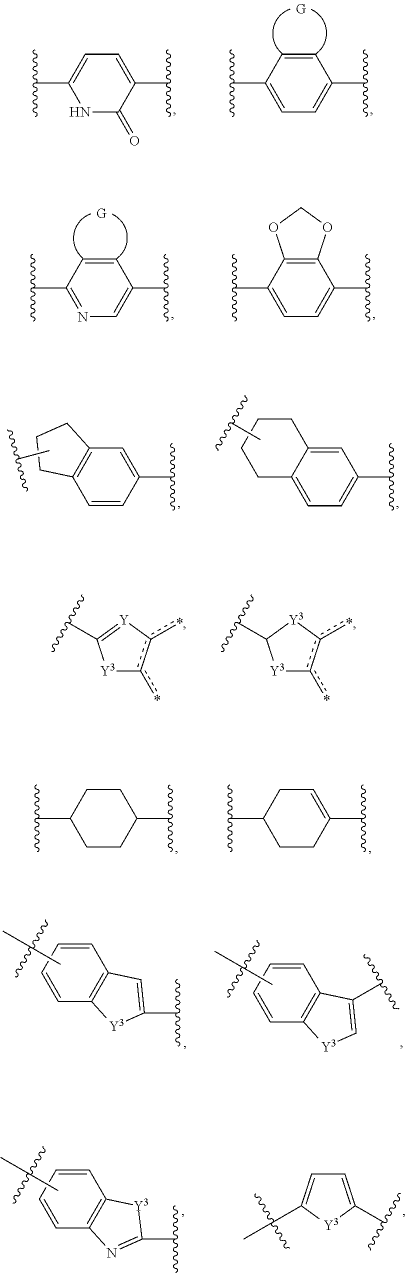 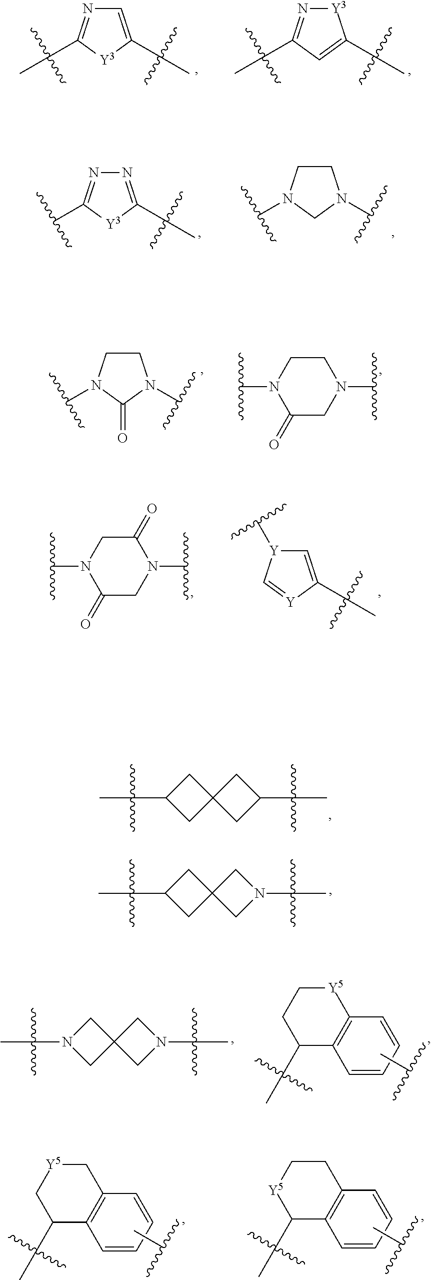 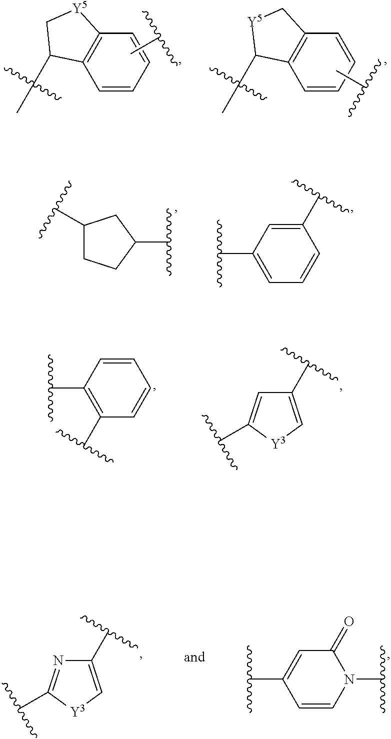
wherein each * is a point of attachment of A to L¹ or L³, and wherein the rings in A are unsubstituted or substituted with one or more substituents selected from alkyl, amino, haloalkyl, halogen, hydroxy, alkoxy, haloalkoxy, cyano, sulfonyl or oxo;
B is an acetylene or selected from the group consisting of 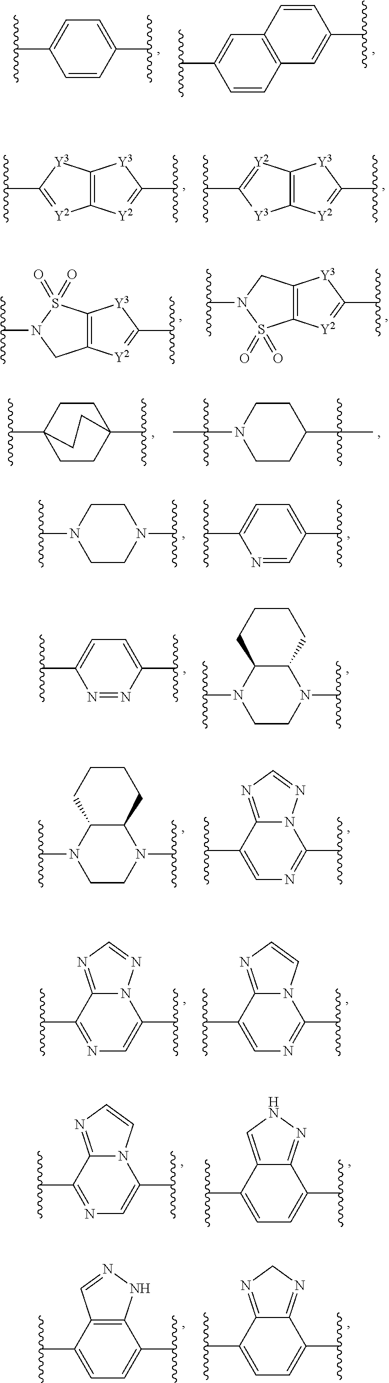 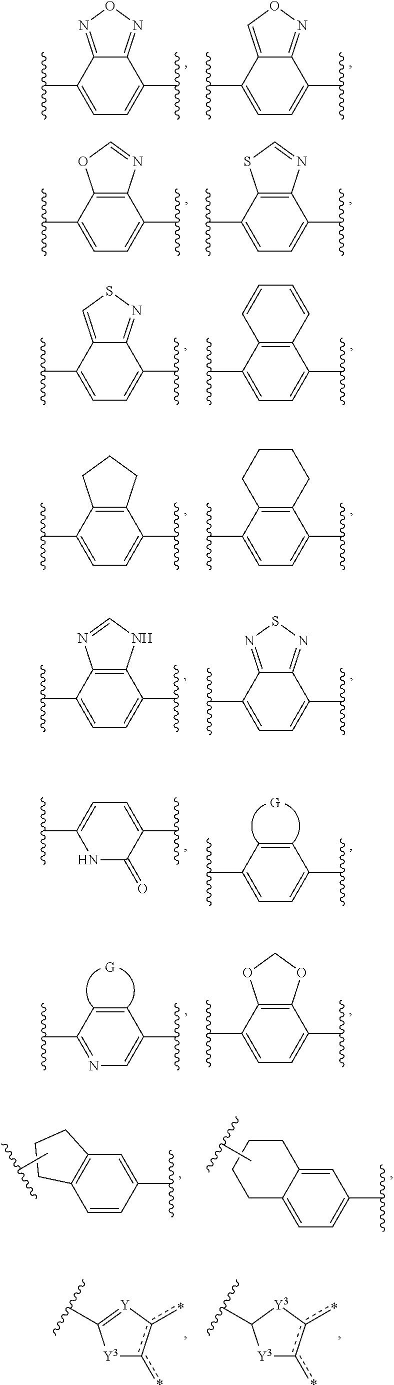 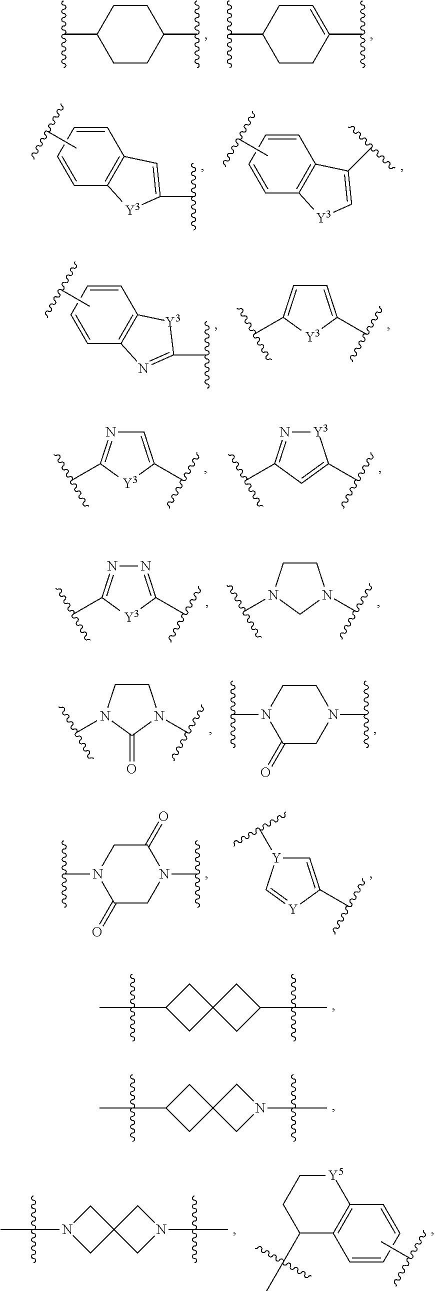 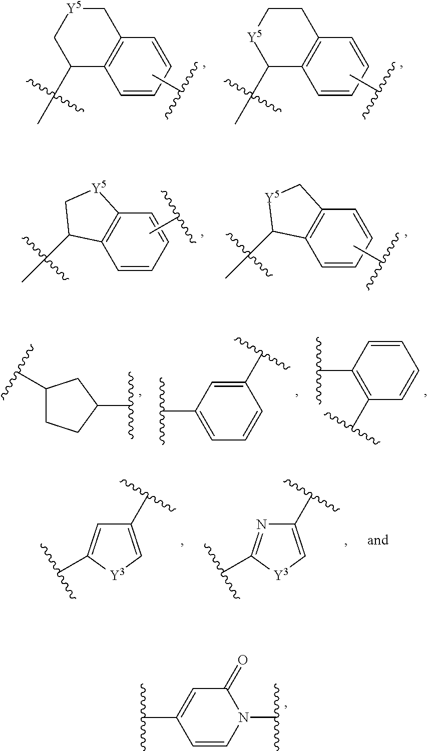
wherein each * is a point of attachment of B to L¹ or L³, wherein the rings in B are unsubstituted or substituted with one or more substituents selected from alkyl, amino, haloalkyl, halogen, hydroxy, alkoxy, haloalkoxy, cyano, sulfonyl or oxo;
G together with the atoms to which it is attached forms a ring system selected from 6-11 membered aryl, 5-11 membered heteroaryl, 4-11 membered heterocyclyl, and 4-11 membered carbocyclyl, wherein the ring system is unsubstituted or substituted with one or more substituents selected from alkyl, amino, haloalkyl, halogen, or oxo; 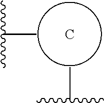
is selected from 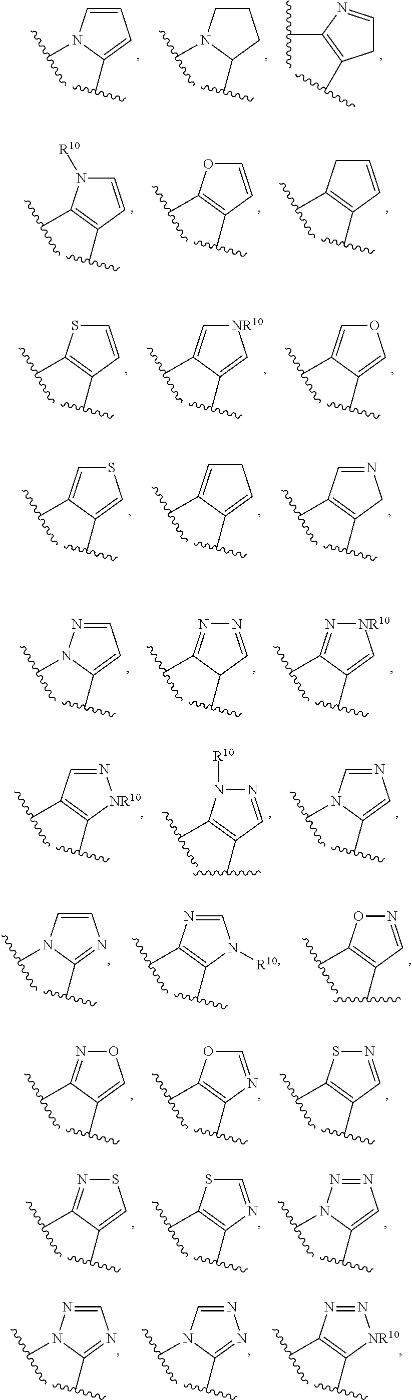 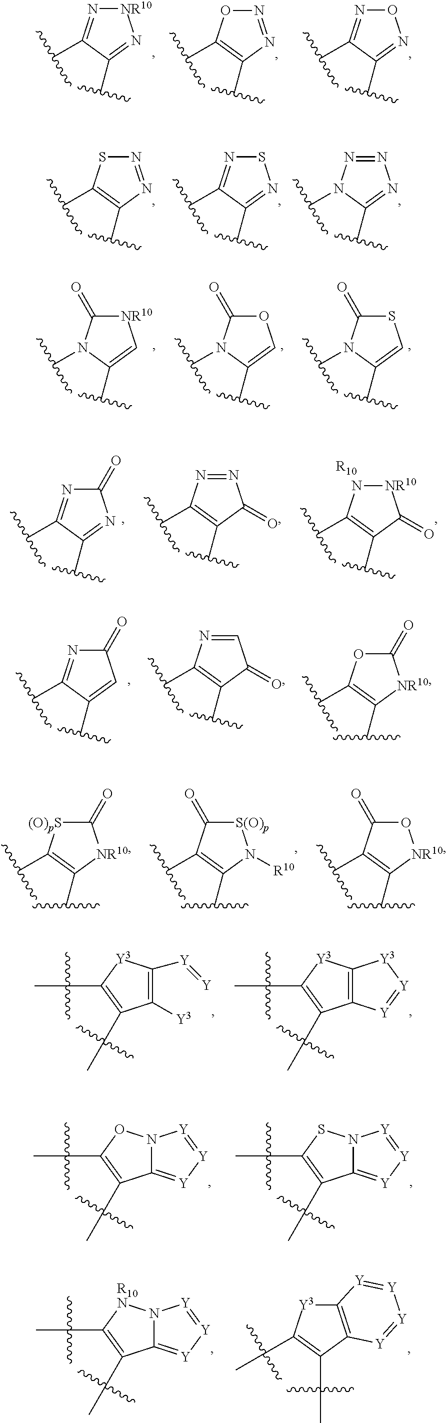 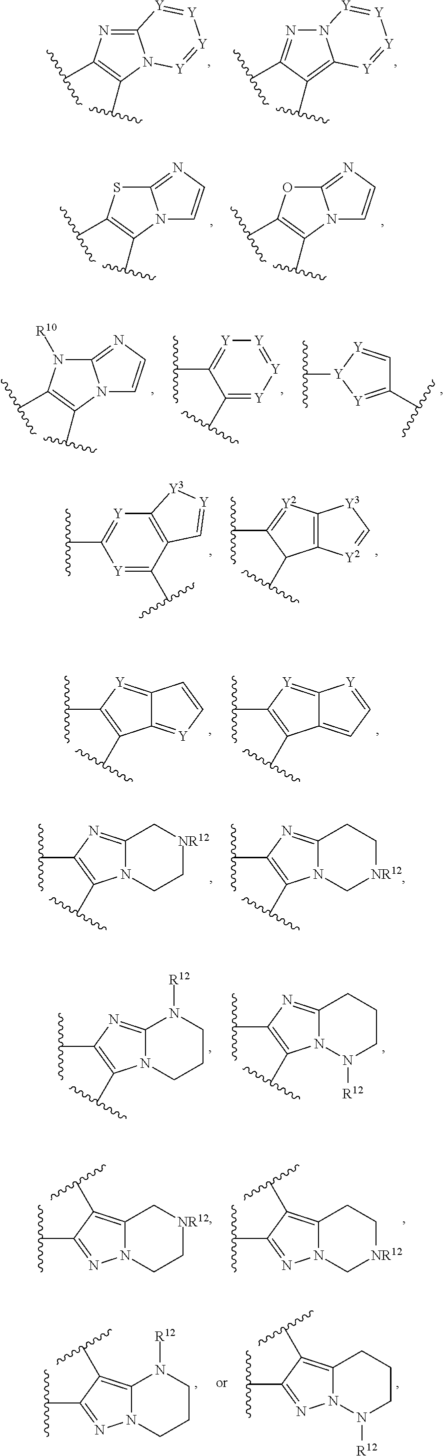
or optionally substituted variants thereof;
each Y² is independently selected from —CH═ or N;
each Y³ is independently selected from C(R⁶)₂, NR⁶, O or S;
each Y⁵ is independently selected from NR⁶, O or S; and
each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

36. The compound or pharmaceutically acceptable salt thereof of claim 33, wherein the compound of Formula (II) is also represented by Formula (IIa) 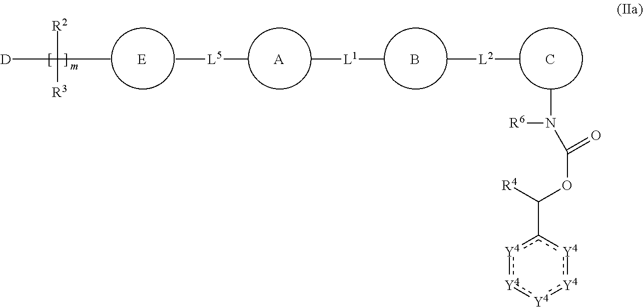
wherein
A is selected from acetylene, 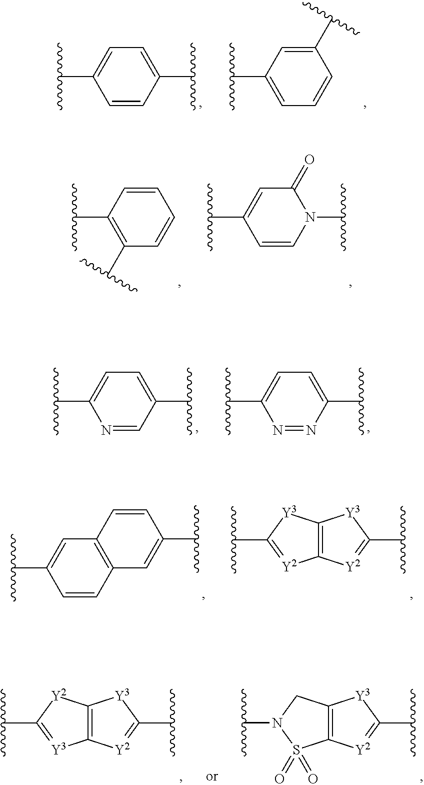
wherein the rings in A are unsubstituted or substituted with one or more substituents selected from alkyl, amino, haloalkyl, halogen, hydroxy, alkoxy, haloalkoxy, cyano, or oxo;
B is selected from acetylene, 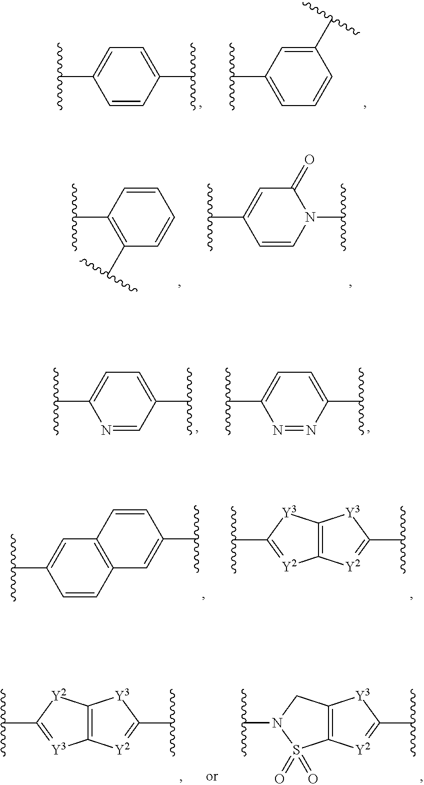
wherein the rings in B are unsubstituted or substituted with one or more substituents selected from alkyl, amino, haloalkyl, halogen, hydroxy, alkoxy, haloalkoxy, cyano, or oxo;
L¹ is selected from a single bond, a —C(O)— linker, a —CH₂— linker, or a —CH₂O-linker;
L² is selected from a single bond, a —O— linker, a —NH— linker, a —C(O)— linker, a CH₂— linker, or a —CH₂O— linker; and
R⁴ is selected from hydrogen or alkyl optionally substituted with halogen.

37. (canceled)

38. The compound or pharmaceutically acceptable salt thereof of claim 33, wherein the compound of Formula (II) is also represented by Formula (IIb): 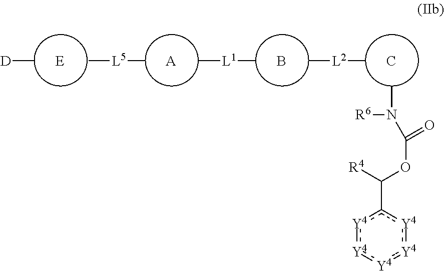
wherein L⁵ is

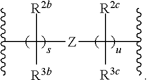

39. The compound or pharmaceutically acceptable salt thereof of claim 33, wherein E is absent.

40. (canceled)

41. The compound or pharmaceutically acceptable salt thereof of claim 33, wherein E is selected from 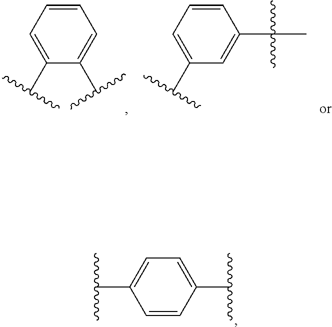 [SMILES: *c1ccccc1* ; *c1cccc(*)c1 ; *c1ccc(*)cc1]
each optionally substituted with one or more halogens.

42. (canceled)

43. The compound or pharmaceutically acceptable salt thereof of claim 33, wherein E is selected from

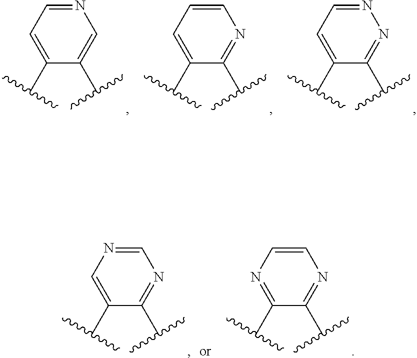 [SMILES: *c1ccncc1* ; *c1cccnc1* ; *c1ccnnc1* ; *c1cncnc1* ; *c1nccnc1*]

44. (canceled)

45. (canceled)

46. The compound or pharmaceutically acceptable salt thereof of claim 33, wherein E is selected from 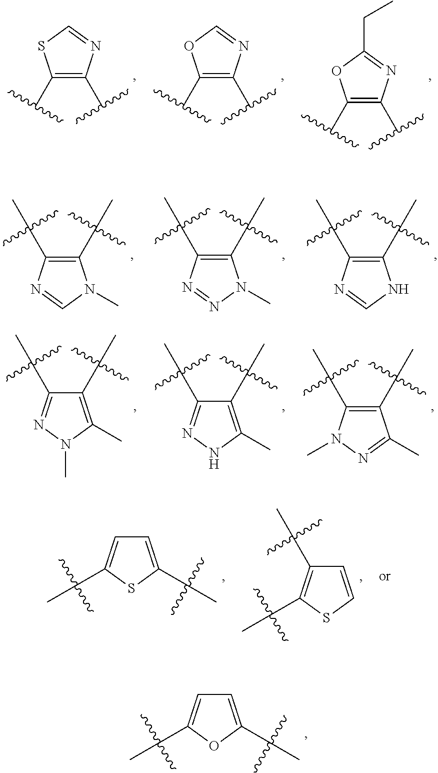
each optionally substituted with one or more substituents selected from alkyl, amino, halogen, haloalkyl, hydroxy, alkoxy, haloalkoxy, cyano, or oxo.

47. (canceled)

48. The compound or pharmaceutically acceptable salt thereof of claim 33, wherein E is selected from 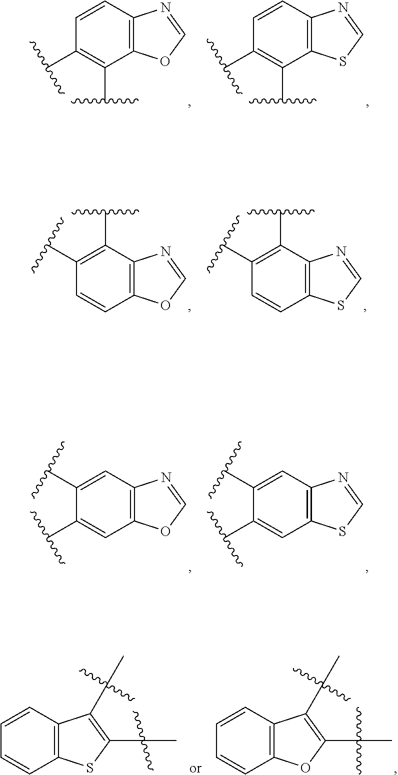
each optionally substituted with one or more substituents selected from alkyl, amino, halogen, haloalkyl, hydroxy, alkoxy, haloalkoxy, cyano, or oxo.

49. (canceled)

50. The compound or pharmaceutically acceptable salt thereof of claim 33, wherein E is selected from 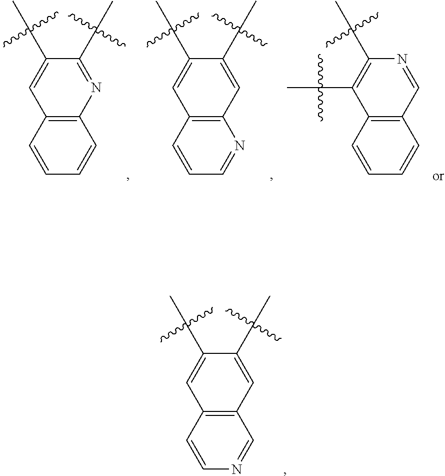 [SMILES: *c1cc2ccccc2nc1* ; *c1cc2cccnc2cc1* ; *c1ncc2ccccc2c1* ; *c1cc2ccncc2cc1*]
each optionally substituted with one or more substituents selected from alkyl, amino, halogen, haloalkyl, hydroxy, alkoxy, haloalkoxy, cyano, or oxo.

51. The compound or pharmaceutically acceptable salt thereof of claim 33, wherein D is —C(O)OR¹.

52. The compound or pharmaceutically acceptable salt thereof of claim 33, wherein the compound of Formula (II) is also represented by Formula (IIc):

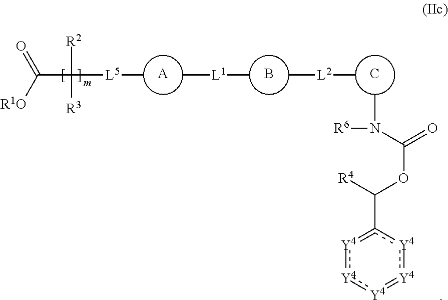

53. The compound or pharmaceutically acceptable salt thereof of claim 33, wherein A is 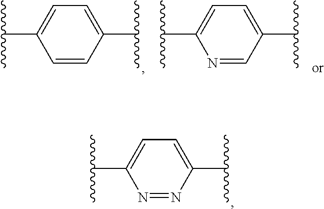
and B is selected from acetylene, 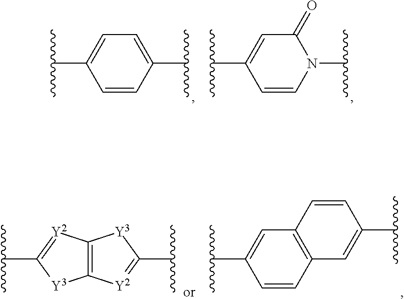
and wherein each of the rings in A or B is unsubstituted or substituted with one or more substituents selected from alkyl, amino, haloalkyl, halogen, hydroxy, alkoxy, haloalkoxy, cyano, or oxo.

54. (canceled)

55. The compound or pharmaceutically acceptable salt thereof of claim 53, wherein B is an acetylene.

56. The compound or pharmaceutically acceptable salt thereof of claim 53, wherein B is 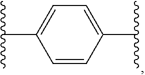 [SMILES: *c1ccc(*)cc1]
optionally substituted with one or more halogens.

57. (canceled)

58. (canceled)

59. The compound or pharmaceutically acceptable salt thereof of claim 53, wherein A is 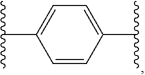 [SMILES: *c1ccc(*)cc1]
optionally substituted with one or more halogens.

60. The compound or pharmaceutically acceptable salt thereof of claim 33, wherein B is 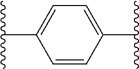 [SMILES: *c1ccc(*)cc1]
and A is selected from acetylene, 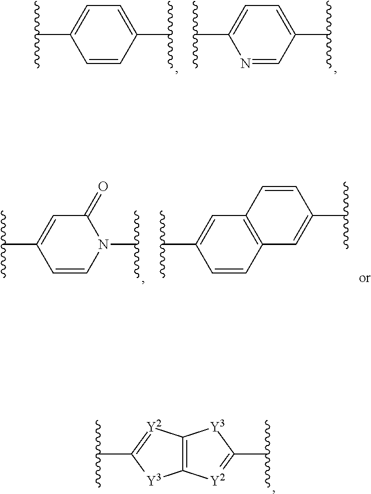
wherein each of the rings in A or B is unsubstituted or substituted with one or more substituents selected from alkyl, amino, haloalkyl, halogen, hydroxy, alkoxy, haloalkoxy, cyano, or oxo.

61. (canceled)

62. The compound or pharmaceutically acceptable salt thereof of claim 60, wherein A is an acetylene.

63. The compound or pharmaceutically acceptable salt thereof of claim 60, wherein A is 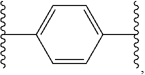 [SMILES: *c1ccc(*)cc1]
optionally substituted with one or more halogens.

64. (canceled)

65. (canceled)

66. (canceled)

67. (canceled)

68. (canceled)

69. The compound or pharmaceutically acceptable salt thereof of claim 33, wherein C is selected from

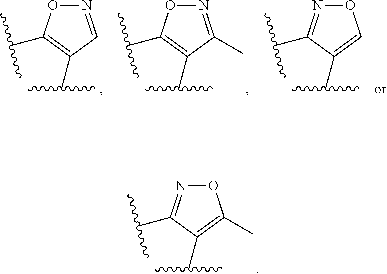

70. The compound or pharmaceutically acceptable salt thereof of claim 33, wherein C is selected from

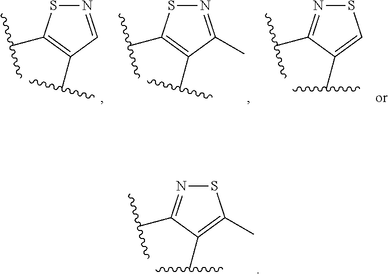

71. The compound or pharmaceutically acceptable salt thereof of claim 33, wherein C is selected from 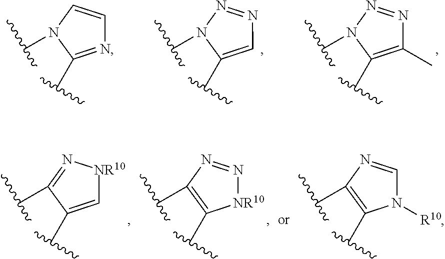
wherein R¹⁰ is selected from hydrogen, C₁₋₃ alkyl or C₃₋₆ cycloalkyl.

72. The compound or pharmaceutically acceptable salt thereof of claim 71, wherein C is selected from

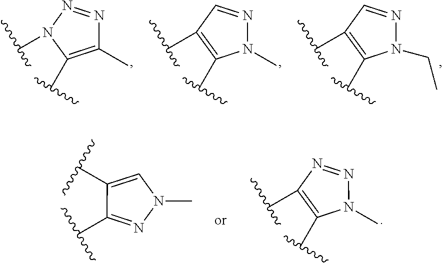

73. (canceled)

74. (canceled)

75. (canceled)

76. (canceled)

77. (canceled)

78. The compound or pharmaceutically acceptable salt thereof of claim 33, wherein m is 0.

79. The compound or pharmaceutically acceptable salt thereof of claim 33, wherein m is 1.

80. (canceled)

81. The compound or pharmaceutically acceptable salt thereof of claim 33, wherein each of R² and R³ is hydrogen.

82. The compound or pharmaceutically acceptable salt thereof of claim 33, wherein at least one of R² and R³ is alkyl, aryl or halogen.

83. The compound or pharmaceutically acceptable salt thereof of claim 82, wherein both R² and R³ are alkyl.

84. The compound or pharmaceutically acceptable salt thereof of claim 82, wherein one of R² or R³ is alkyl and the other R² or R³ is halogen.

85. The compound or pharmaceutically acceptable salt thereof of claim 82, wherein both R² and R³ are halogens.

86. The compound or pharmaceutically acceptable salt thereof of claim 33, wherein R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl.

87. The compound or pharmaceutically acceptable salt thereof of claim 86, wherein R² and R³ are joined together with the atom to which they are attached to form an optionally substituted cyclopropyl, cyclobutyl or cyclopentyl.

88. (canceled)

89. The compound or pharmaceutically acceptable salt thereof of claim 33, wherein R⁶ is hydrogen.

90. The compound or pharmaceutically acceptable salt thereof of claim 33, wherein each of L¹ and L² is a single bond.

91. (canceled)

92. The compound or pharmaceutically acceptable salt thereof of claim 33, wherein L⁵ is

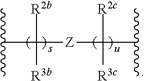

93. (canceled)

94. The compound or pharmaceutically acceptable salt thereof of claim 92, wherein both s and u in L⁵ are an integer of 0.

95. The compound or pharmaceutically acceptable salt thereof of claim 94, wherein L⁵ is —NH—.

96. The compound or pharmaceutically acceptable salt thereof claim 94, wherein L⁵ is —C(O)—NH—.

97. The compound or pharmaceutically acceptable salt thereof of claim 94, wherein L⁵ is —O—.

98. (canceled)

99. (canceled)

100. (canceled)

101. The compound or pharmaceutically acceptable salt thereof of claim 33, wherein R¹ is hydrogen or alkyl.

102. (canceled)

103. The compound or pharmaceutically acceptable salt thereof of claim 33, wherein

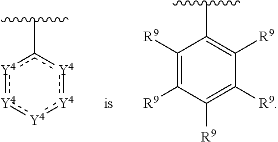

104. (canceled)

105. (canceled)

106. (canceled)

107. The compound or pharmaceutically acceptable salt thereof of claim 33, selected from compounds of Tables 2, 2A, 2B, 2C and 2D, and pharmaceutically acceptable salts thereof.

108. The compound or pharmaceutically acceptable salt thereof of claim 33, selected from compounds IT005, IT006, IT155, IT194-IT199, IT226-IT232, IT238, IT256-259, IT277, IT300, IT301, IT303-IT316, IT344, IT345, IT355, IT356, IT368, IT374, IT375, IT388, IT398-IT409, IT417, IT419, IT420, IT423-IT425, IT428-IT432, IT434-IT440, IT444, IT446-IT457, IT459-IT474, IT476-IT478, IT481-IT492, IT495, IT497, or IT500-IT514 of Table 13.

109.-440. (canceled)

441. A compound of Formula (XI): 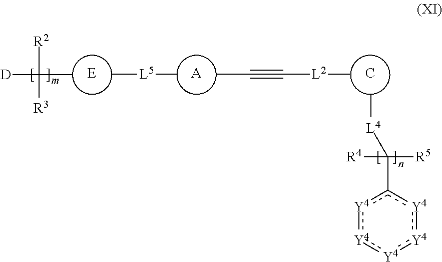
or a pharmaceutically acceptable salt thereof, wherein
A is selected from the group consisting of 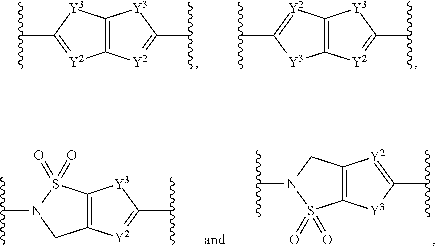
wherein A is optionally substituted; 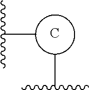
is selected from 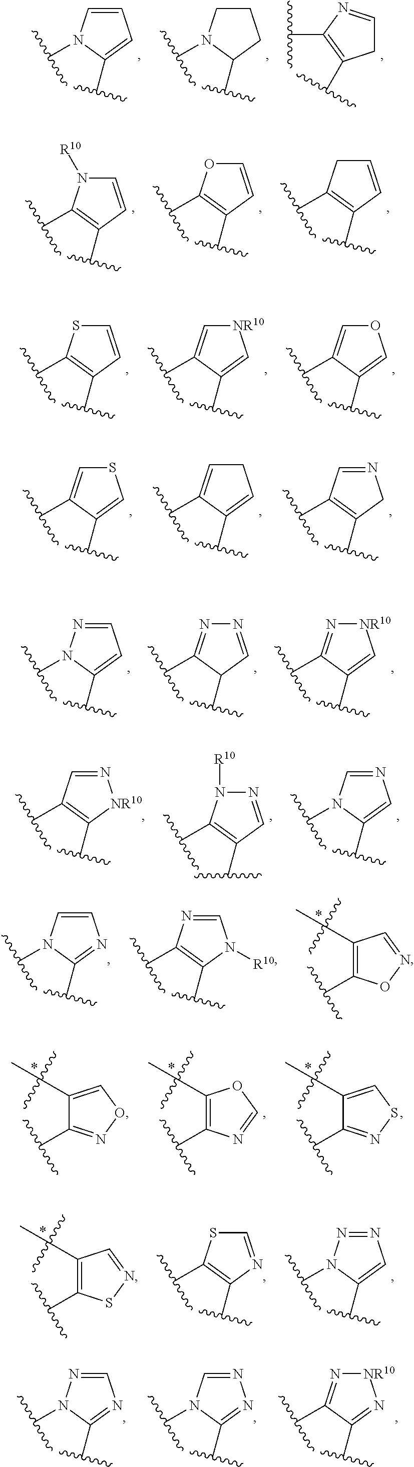 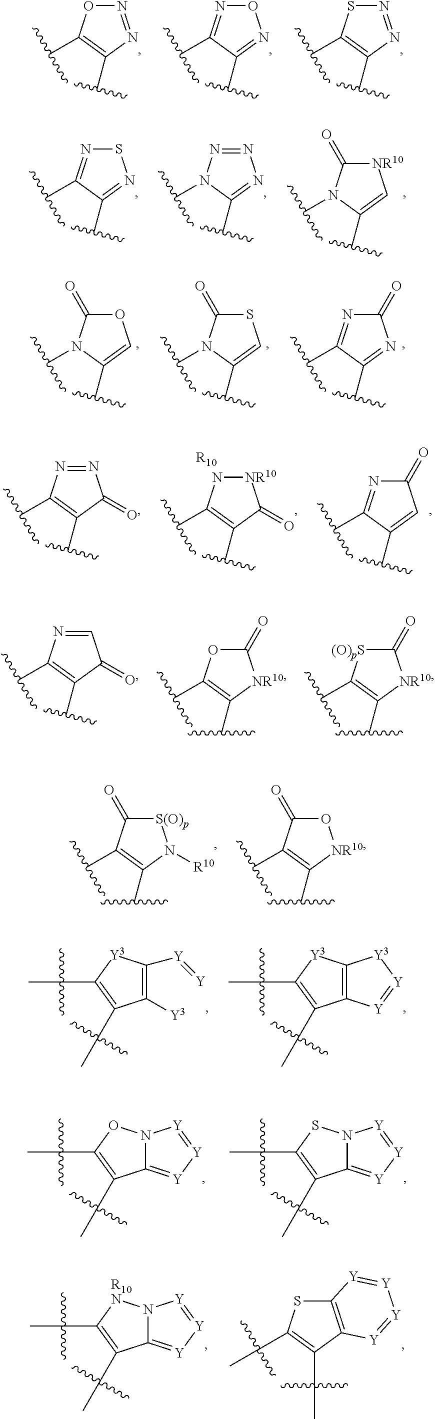 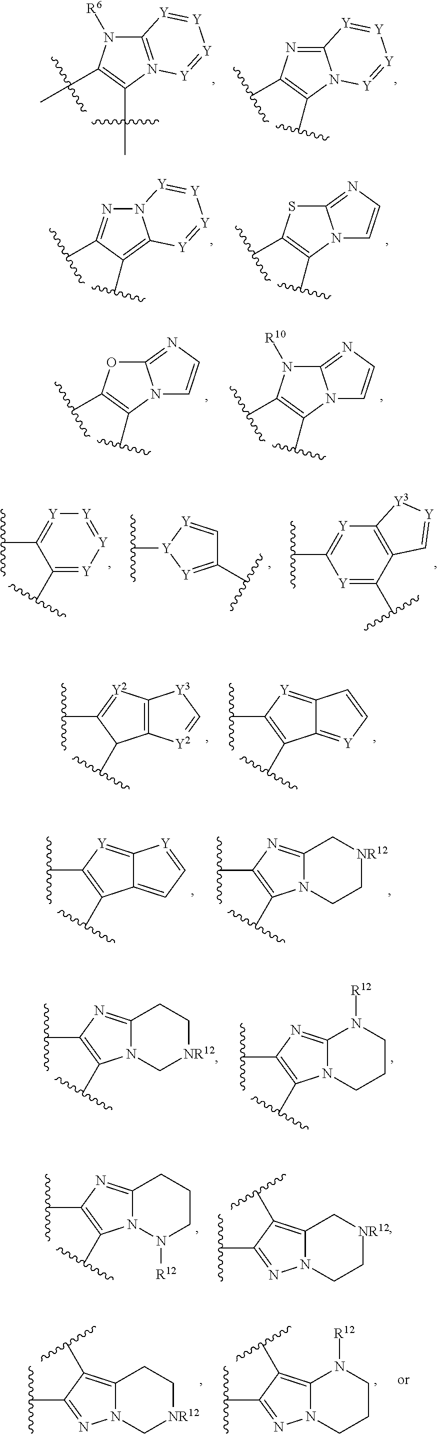 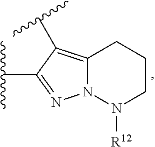
or optionally substituted variants thereof; wherein each * is a point of attachment of C to L²;
D is selected from —OH, 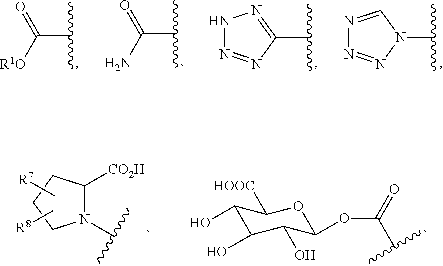
—NR¹³SOₚR¹⁴, —C(O)—NR¹³SOₚR¹⁴, 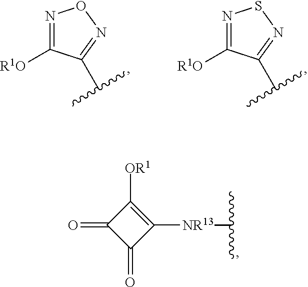
—SOₚR¹⁵, —SOₚNR¹⁶R¹⁷, or carboxylic acid isosteres;
E is absent or selected from 6-10 membered arylene, 3-11 membered carbocyclyl, 3-11 membered heterocyclyl or 5 to 10 membered heteroarylene, wherein E is optionally substituted;
L⁴ is selected from 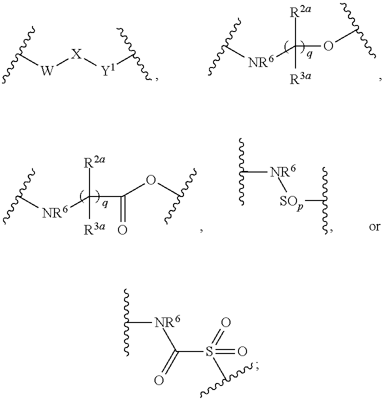
or alternatively, 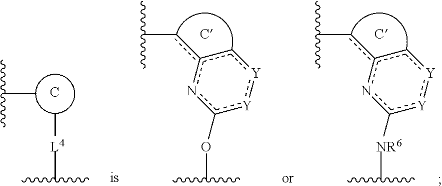
wherein 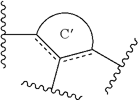
is selected from: 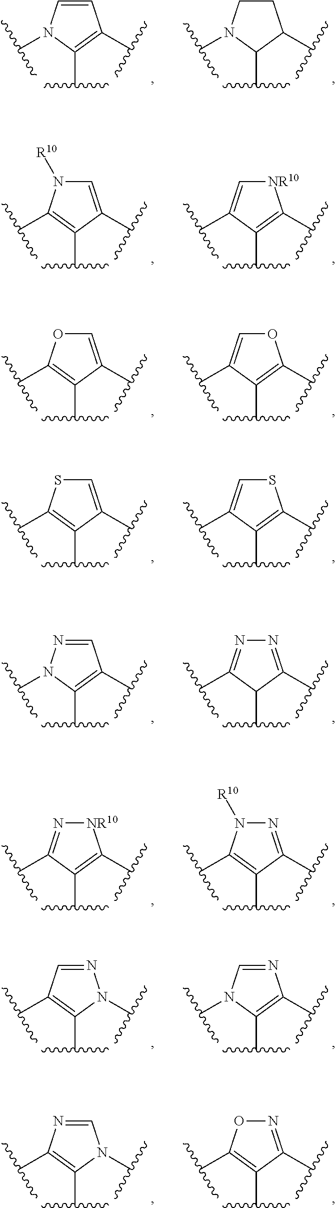 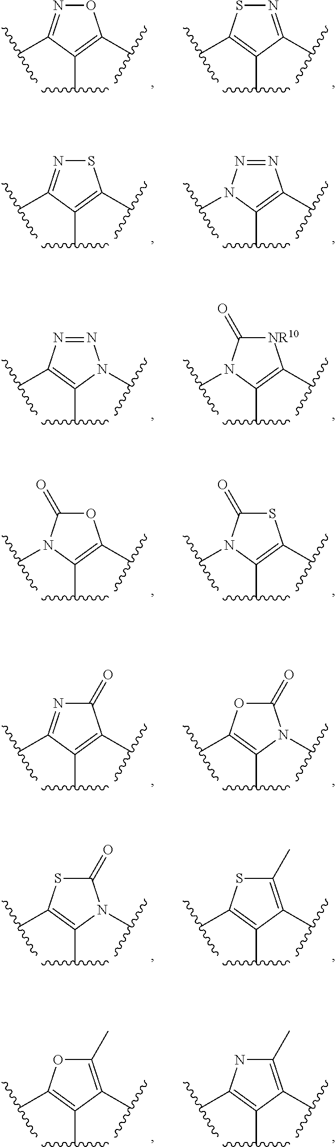 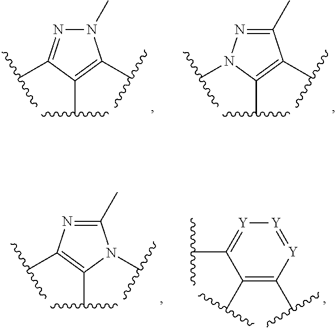
or optionally substituted variants thereof;
L² is selected from a single bond, a —CH₂— linker, 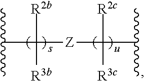
a —C≡C— linker, or a —CH═CH— linker;
L⁵ is selected from a single bond, a —CH═CH— linker, a —C≡C— linker, 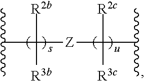
or a 4-7 membered heterocyclyl;
W is selected from C(R⁶)₂, NR⁶, or O;
X is selected from —C(O) or S(O)ₚ;
each Y is independently selected from CR or N;
Y¹ is selected from C(R⁶)₂, NR⁶, or O;
each Y² is independently selected from —CH═ or N;
each Y³ is independently selected from C(R⁶)₂, NR⁶, O or S;
each Y⁴ is independently absent, CR⁹, C(R⁹)₂, N, or NH, provided that only one Y⁴ can be absent;
each Z is independently selected from C(O), O, S, S(O)₂, NR^{6a}, C(O)NR^{6b}, or S(O)₂NR^{6c};
R¹ is selected from hydrogen or alkyl optionally substituted with one or more substituents selected from the group consisting of halogen, hydroxy, alkoxy, C-amido, O-carboxy, and 5-7 membered heterocyclyl, or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;
R² and R³ are each independently selected from hydrogen, alkyl, halogen, haloalkyl, cycloalkyl, heterocyclyl, aryl, or heteroaryl; wherein each of cycloalkyl, heterocyclyl, aryl, and heteroaryl of R² or R³ is optionally substituted with one or more substituents selected from alkyl, amino, halogen, haloalkyl, hydroxy, alkoxy, haloalkoxy, cyano, or oxo;
or R² and R³ are joined together with the atom to which they are attached to form an optionally substituted cycloalkyl or an optionally substituted heterocyclyl;
or R² is selected from hydrogen, alkyl, halogen, haloalkyl, cycloalkyl, heterocyclyl, aryl, or heteroaryl and R³ is joined to an atom alpha to a point of attachment of L⁵ to A to form an optionally substituted cycloalkyl or an optionally substituted heterocyclyl when E is absent; or R³ is selected from hydrogen, alkyl, halogen, haloalkyl, cycloalkyl, heterocyclyl, aryl or heteroaryl and R² is joined to an atom alpha to a point of attachment of L⁵ to A to form an optionally substituted cycloalkyl or an optionally substituted heterocyclyl when E is absent;
each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;
each R⁶, R^{6a}, R^{6b}, and R^{6c} is independently selected from hydrogen, alkyl optionally substituted with one or more substituents selected from the group consisting of halogen, hydroxy and alkoxy, halogen, aryl, or C₃₋₆ cycloalkyl;
each R⁷ and R⁸ is independently selected from hydrogen or C₁₋₆ alkyl optionally substituted with one or more substituents selected from the group consisting of halogen, hydroxy and alkoxy, or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl optionally substituted with one or more substituents selected from the group consisting of halogen, hydroxy and alkoxy, or halogen, or two adjacent R⁹ are joined together with the atoms to which they are attached to form an optionally substituted carbocyclyl or an optionally substituted heterocyclyl;
each R¹⁰ is independently selected from hydrogen, alkyl optionally substituted with one or more substituents selected from the group consisting of halogen, hydroxy and alkoxy, halogen, aryl, C₃₋₆ cycloalkyl, or cyano;
each R¹² is independently selected from hydrogen, alkyl optionally substituted with one or more substituents selected from the group consisting of halogen, hydroxy and alkoxy, acyl, C-carboxy, C-amido, sulfinyl, sulfonyl, or S-sulfonamido.
each R¹³ and R¹⁴ is independently selected from hydrogen, alkyl, haloalkyl, halogen, aryl, or C₃₋₆ cycloalkyl;
R¹⁵ is selected from hydrogen, alkyl, haloalkyl, (carbocyclyl)alkyl, aryl, or C₃₋₆ cycloalkyl;
each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;
each R^{2a}, R^{3a}, R^{2b}, R^{3b}, R_{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;
m is independently an integer from 0-3;
n is an integer from 0-3;
p is an integer from 1-2;
q is an integer from 1-6;
each s and u is independently an integer from 0 to 6; and [symbol]
represents a single or double bond.

442. (canceled)

443. The compound or pharmaceutically acceptable salt thereof of claim 441, wherein
C is selected from the group consisting of 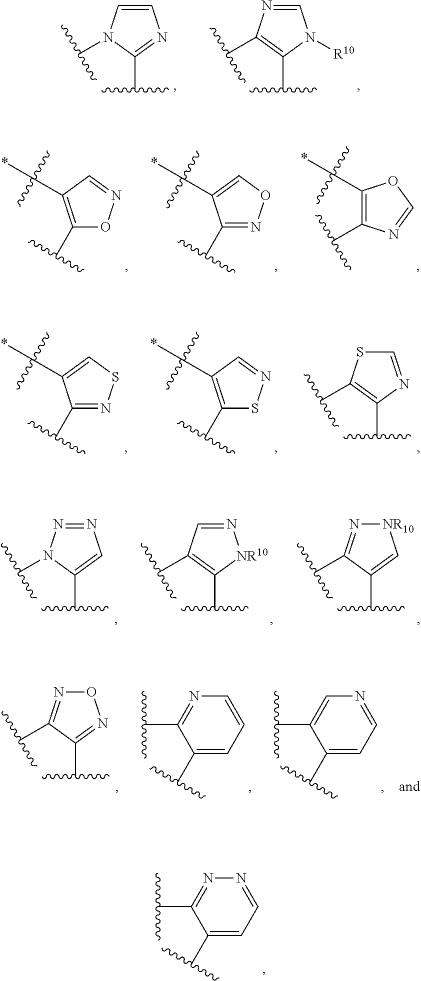
wherein C is unsubstituted or substituted with one or more substituents selected from C₁₋₃ alkyl optionally substituted with halogen or C₁₋₃ alkoxy, C₁₋₆ alkoxy, C₃₋₆ cycloalkyl, halogen, or cyano.

444. The compound or pharmaceutically acceptable salt thereof of claim 441, wherein the compound of Formula (XI) is also represented by Formula (XIa): 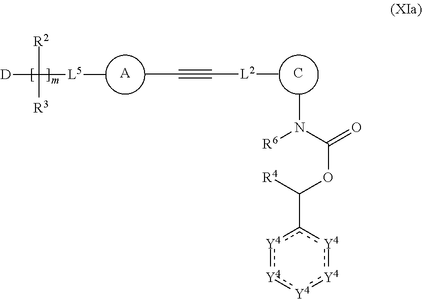
wherein A is selected from the group consisting of 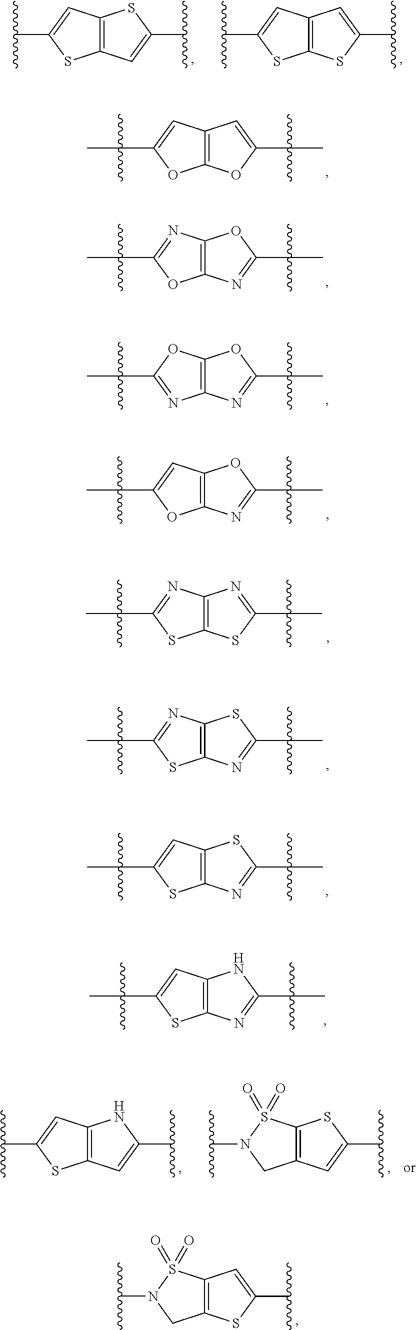
wherein the rings in A are unsubstituted or substituted with one or more substituents selected from alkyl, amino, haloalkyl, halogen, hydroxy, alkoxy, haloalkoxy, cyano, or oxo.

445. (canceled)

446. The compound or pharmaceutically acceptable salt thereof of claim 441, wherein A is selected from 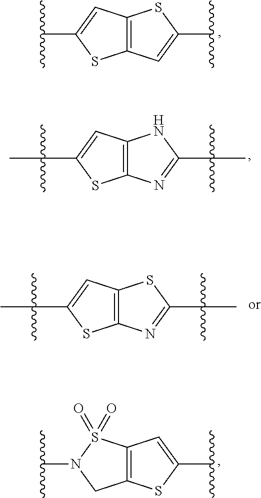 [SMILES: *c1cc2sc(*)cc2s1 ; *c1nc2sc(*)cc2[nH]1 ; *c1cc2sc(*)nc2s1 ; *c1cc2c(s1)CN(*)S2(=O)=O]
each unsubstituted or substituted with one or more substituents selected from alkyl, haloalkyl, halogen, hydroxy, alkoxy, haloalkoxy, cyano, or oxo.

447. The compound or pharmaceutically acceptable salt thereof of claim 441, wherein A is optionally substituted

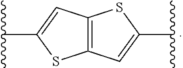 [SMILES: *c1cc2sc(*)cc2s1]

448. The compound or pharmaceutically acceptable salt thereof of claim 441, wherein D is —C(O)OR¹.

449. The compound or pharmaceutically acceptable salt thereof of claim 448, wherein R¹ is hydrogen or unsubstituted alkyl.

450. The compound or pharmaceutically acceptable salt thereof of claim 448, wherein R¹ is alkyl substituted with one or more substituents selected from the group consisting of alkoxy, C-amido, O-carboxy, and 6 membered heterocyclyl, or optionally substituted aryl.

451. (canceled)

452. The compound or pharmaceutically acceptable salt thereof of claim 441, wherein m is 0.

453. The compound or pharmaceutically acceptable salt thereof of claim 441, wherein m is 1.

454. (canceled)

455. The compound or pharmaceutically acceptable salt thereof of claim 441, wherein each of R² and R³ is hydrogen.

456. The compound or pharmaceutically acceptable salt thereof of claim 441, wherein one of R² or R³ is hydrogen and the other R² or R³ is alkyl or aryl.

457. The compound or pharmaceutically acceptable salt thereof of claim 441, wherein R² and R³ are joined together with the atom to which they are attached to form an optionally substituted azetidine, an optionally substituted oxetane, an optionally substituted beta-lactam, an optionally substituted tetrahydropyran, an optionally substituted cyclopropyl, an optionally substituted cyclobutyl, an optionally substituted cyclopentyl, or an optionally substituted cyclohexyl.

458. The compound or pharmaceutically acceptable salt thereof of claim 457, wherein R² and R³ are joined together with the atom to which they are attached to form an optionally substituted cyclopropyl.

459. The compound or pharmaceutically acceptable salt thereof of claim 441, wherein R⁶ is hydrogen.

460. The compound or pharmaceutically acceptable salt thereof of claim 441, wherein R⁶ is C₁₋₃ alkyl.

461. The compound or pharmaceutically acceptable salt thereof of claim 441, wherein each of L⁵ and L² is a single bond.

462. (canceled)

463. The compound or pharmaceutically acceptable salt thereof of claim 441, wherein R⁴ is alkyl or haloalkyl.

464. (canceled)

465. The compound or pharmaceutically acceptable salt thereof of claim 441, wherein

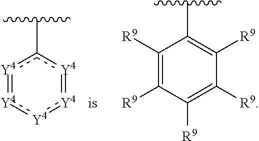

466. (canceled)

467. (canceled)

468. (canceled)

469. The compound or pharmaceutically acceptable salt thereof of claim 441, selected from compounds of Table 10A.

470. The compound or pharmaceutically acceptable salt thereof of claim 441, selected from compounds IT017, IT070, IT082-IT090, IT095, IT097, IT098, IT100, IT101, IT103, IT104, IT106, IT107 IT108, IT109, IT110, IT114 IT115, and IT116, IT118, and IT127 of Table 13.

471. A compound of Formula (XII): 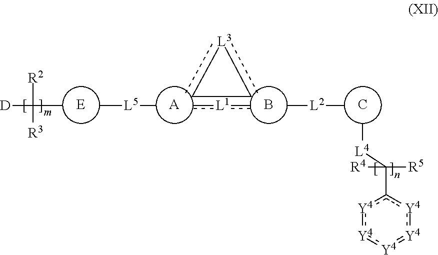
or a pharmaceutically acceptable salt thereof, wherein:
A is an acetylene or a ring system selected from the group consisting of 6-11 membered aryl, 5-11 membered heteroaryl, 4-11 membered heterocyclyl, and 4-11 membered carbocyclyl, wherein when A is a ring system, it is optionally substituted;
B is an acetylene or a ring system selected from the group consisting of 6-11 membered aryl, 5-11 membered heteroaryl, 4-11 membered heterocyclyl, and 4-11 membered carbocyclyl, wherein when B is a ring system, it is optionally substituted;
C is a ring system selected from the group consisting of 6-11 membered aryl, 5-11 membered heteroaryl, 5-11 membered heterocyclyl, and 5-11 membered carbocyclyl, wherein C is optionally substituted;
D is selected from —OH, 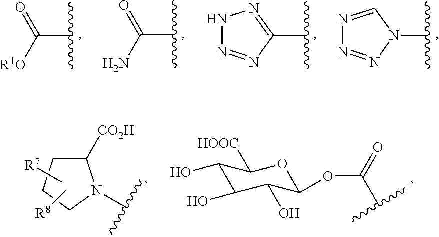
—NR¹³SOₚR¹⁴, —C(O)—NR¹³SOₚR¹⁴, 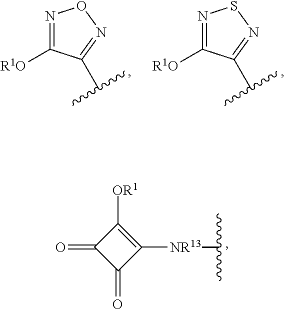
—SOₚR¹⁵, —SOₚNR¹⁶R¹⁷, or carboxylic acid isosteres;
E is absent or selected from 6-10 membered arylene, 3-11 membered carbocyclyl, 3-11 membered heterocyclyl or 5 to 10 membered heteroarylene, wherein E is optionally substituted;
L⁴ is selected from 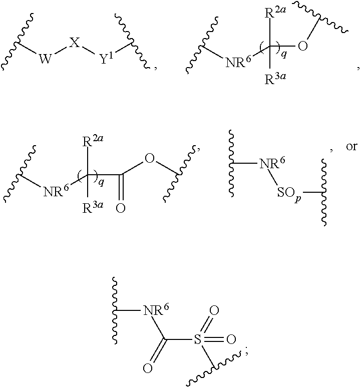
or alternatively, 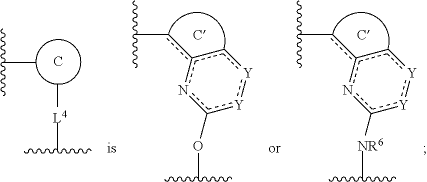
wherein 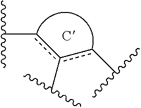
is selected from: 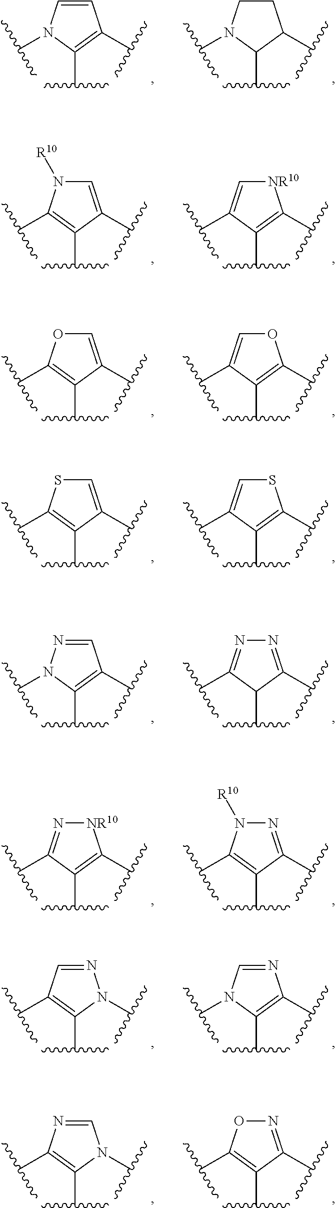 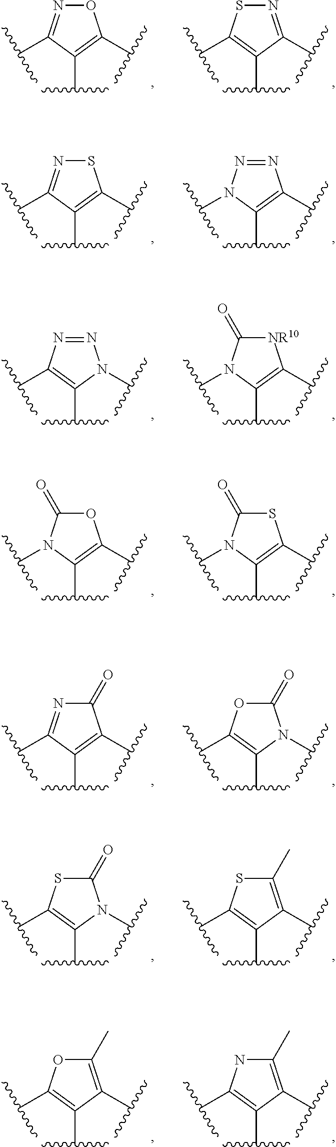 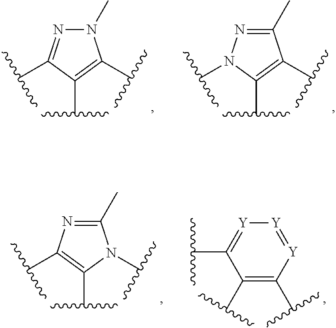
or optionally substituted variants thereof;
L¹ is selected from a single bond, a —CH₂— linker, 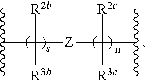
a —C≡C— linker, a —CH═CH— linker, or a ═C(R¹¹)— linker;
L² is selected from a single bond, a —CH₂— linker, 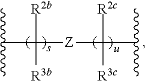
a —C≡C— linker, or a —CH═CH— linker;
L³ is absent or selected from 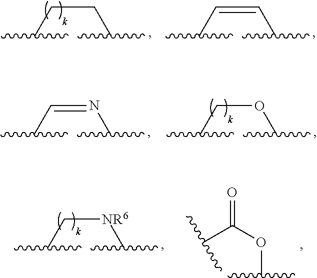
or a ═C(R¹¹)— linker;
L⁵ is selected from a single bond, a —CH═CH— linker, a —C≡C— linker, 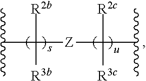
or a 4-7 membered heterocyclyl;
W is selected from C(R⁶)₂, NR⁶, or O;
X is selected from —C(O) or S(O)ₚ;
each Y is independently selected from CR⁶ or N;
Y¹ is C(R⁶)₂, NR⁶, or O;
each Y⁴ is independently absent, CR⁹, C(R⁹)₂, N, or NH, provided that only one Y⁴ can be absent;
each Z is independently selected from C(O), O, S, S(O)₂, NR^{6a}, C(O)NR^{6b}, or S(O)₂NR^{6c};
R¹ is selected from hydrogen; alkyl optionally substituted with one or more substituents selected from the group consisting of halogen, hydroxy, alkoxy, C-amido, O-carboxy, and 5-7 membered heterocyclyl; or aryl optionally substituted with one or more substituents selected from group consisting of amino, cyano, halogen, alkyl, haloalkyl, hydroxy, alkoxy, haloalkoxy, C-amido, N-amino, C-carboxy, O-carboxy and nitro;
R² and R³ are each independently selected from hydrogen, halogen, haloalkyl, C₃₋₇ cycloalkyl, 3-7 membered heterocyclyl, or 5-10 membered heteroaryl; wherein each C₃₋₇ cycloalkyl, 3-7 membered heterocyclyl, and 5-10 membered heteroaryl of R² or R³ is optionally substituted; provided that R² and R³ cannot both be hydrogen;
or R² and R³ are joined together with the atom to which they are attached to form a halo-substituted C₃₋₇ cycloalkyl or halo-substituted 3-7 membered heterocyclyl;
each R⁴ and R⁵ is independently selected from hydrogen or alkyl optionally substituted with one or more substituents selected from the group consisting of halogen, hydroxy and alkoxy; or R⁴ and R⁵ are joined together with the atom to which they are attached to form an optionally substituted cycloalkyl or optionally substituted heterocyclyl;
each R⁶, R^{6a}, R^{6b}, and R^{6c} is independently selected from hydrogen; alkyl optionally substituted with one or more substituents selected from the group consisting of halogen, hydroxy and alkoxy; halogen; aryl; or C₃₋₆ cycloalkyl;
each R⁷ and R⁸ is independently selected from hydrogen or C₁₋₆ alkyl optionally substituted with one or more substituents selected from the group consisting of halogen, hydroxy and alkoxy; or R⁷ and R⁸ are joined together with the atom or atoms to which they are attached to form a spirocyclic heterocyclyl, a spirocyclic carbocyclyl, a fused heterocycle, or a fused carbocyclyl;
each R⁹ is independently selected from hydrogen, alkyl optionally substituted with one or more substituents selected from the group consisting of halogen, hydroxy and alkoxy, or halogen; or two adjacent R⁹ are joined together with the atoms to which they are attached to form an optionally substituted carbocyclyl or an optionally substituted heterocyclyl;
each R¹⁰ is independently selected from hydrogen, alkyl optionally substituted with one or more substituents selected from the group consisting of halogen, hydroxy and alkoxy; halogen; aryl; C₃₋₆ cycloalkyl; or cyano;
each R¹¹ is independently selected from hydrogen, alkyl optionally substituted with one or more substituents selected from the group consisting of halogen, hydroxy and alkoxy; halogen; haloalkyl; or cyano;
each R¹³ and R¹⁴ is independently selected from hydrogen, alkyl, haloalkyl, aryl, or C₃₋₆ cycloalkyl;
R¹⁵ is selected from hydrogen, alkyl, haloalkyl, (carbocyclyl)alkyl, aryl, or C₃₋₆ cycloalkyl
each R¹⁶ and R¹⁷ is independently selected from hydrogen, acyl, alkyl, haloalkyl, aryl, or C₃₋₆ cycloalkyl; or R¹⁶ and R¹⁷ are joined together with the atom to which they are attached to form an optionally substituted cycloalkyl or an optionally substituted heterocyclyl;
each R^{2a}, R^{3a}, R^{2b}, R^{3b}, R^{2c}, and R^{3c} is independently selected from hydrogen, alkyl, halogen, haloalkyl, cycloalkyl, heterocyclyl, aryl, or heteroaryl; or each of R^{2a} and R^{3a}, R^{2b} and R^{3b}, or R^{2c} and R^{3c} are independently joined together with the atom to which they are attached to form an optionally substituted cycloalkyl or an optionally substituted heterocyclyl;
m is independently an integer from 1-3;
n is an integer from 0-3;
k is an integer from 0-3;
p is an integer from 1-2;
q is an integer from 1-6;
each s and u is independently an integer from 0 to 6; and [symbol]
represents a single or double bond.

472. (canceled)

473. The compound or pharmaceutically acceptable salt thereof of claim 471, wherein
A is an acetylene or selected from the group consisting of 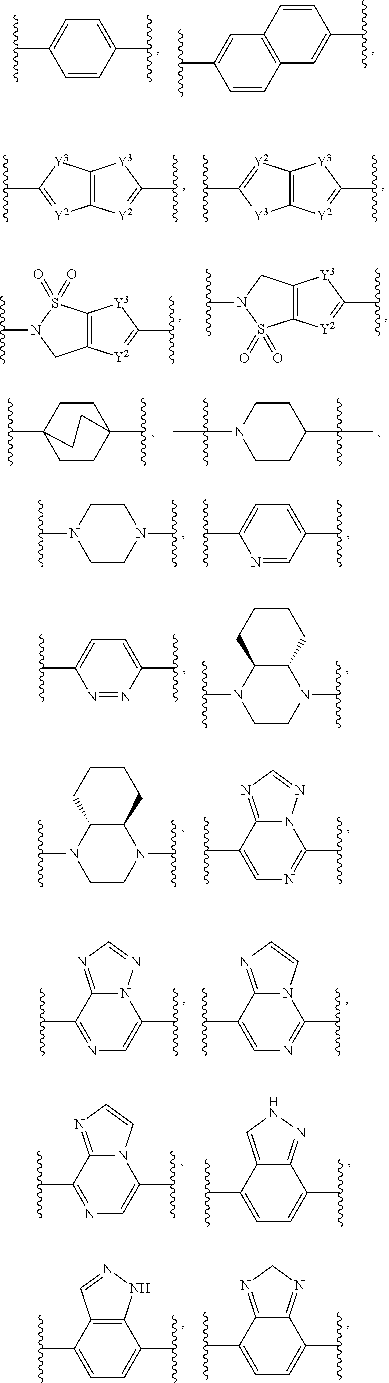 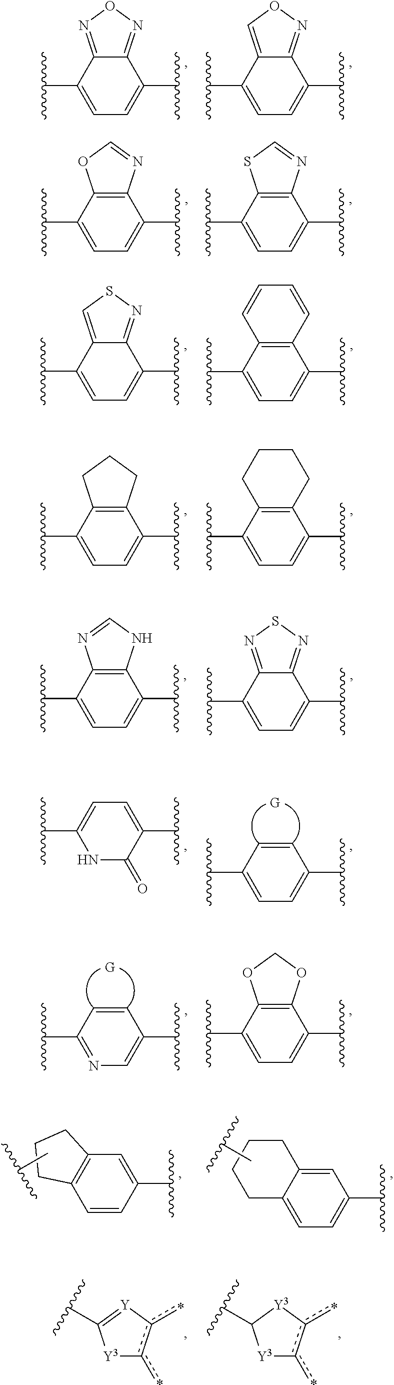 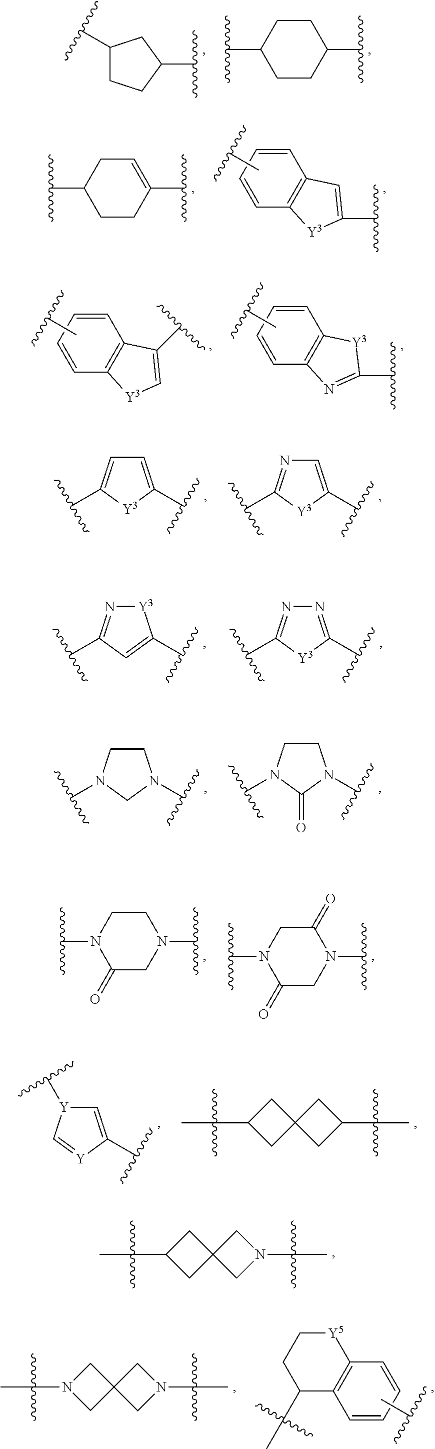 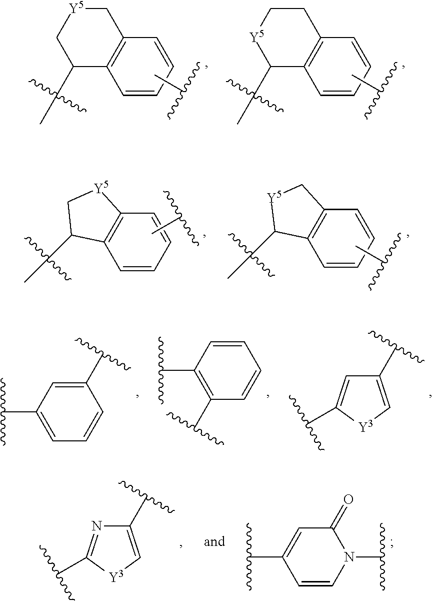
wherein each * is a point of attachment of A to L¹ or L³, and wherein the rings in A are optionally substituted with one or more substituents selected from alkyl, amino, haloalkyl, halogen, hydroxy, alkoxy, haloalkoxy, cyano, sulfonyl or oxo;
B is an acetylene or selected from the group consisting of 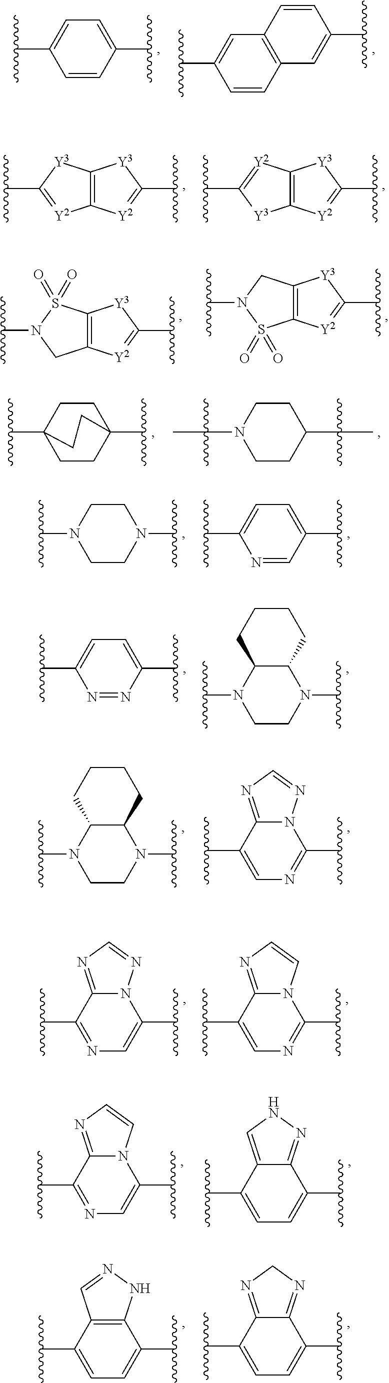 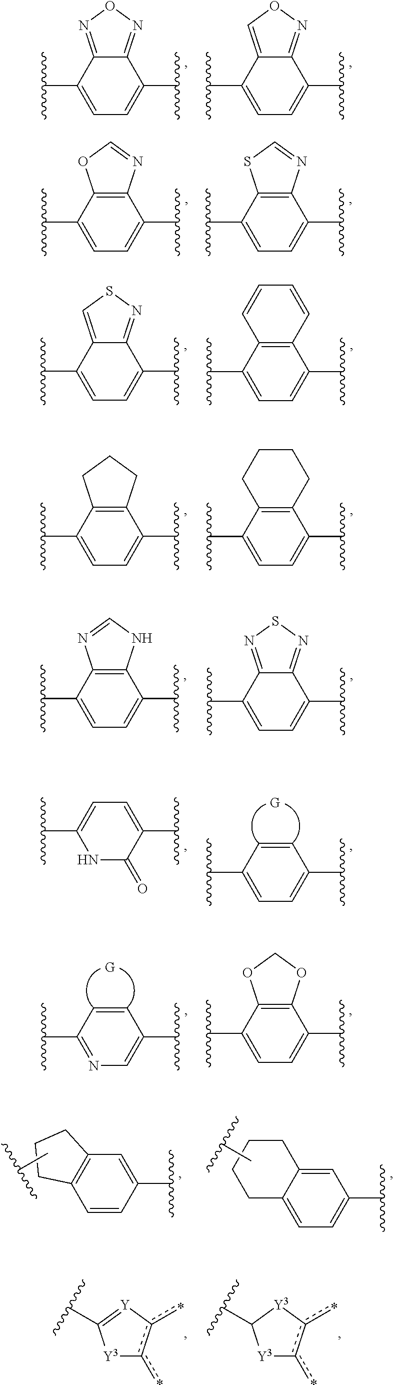 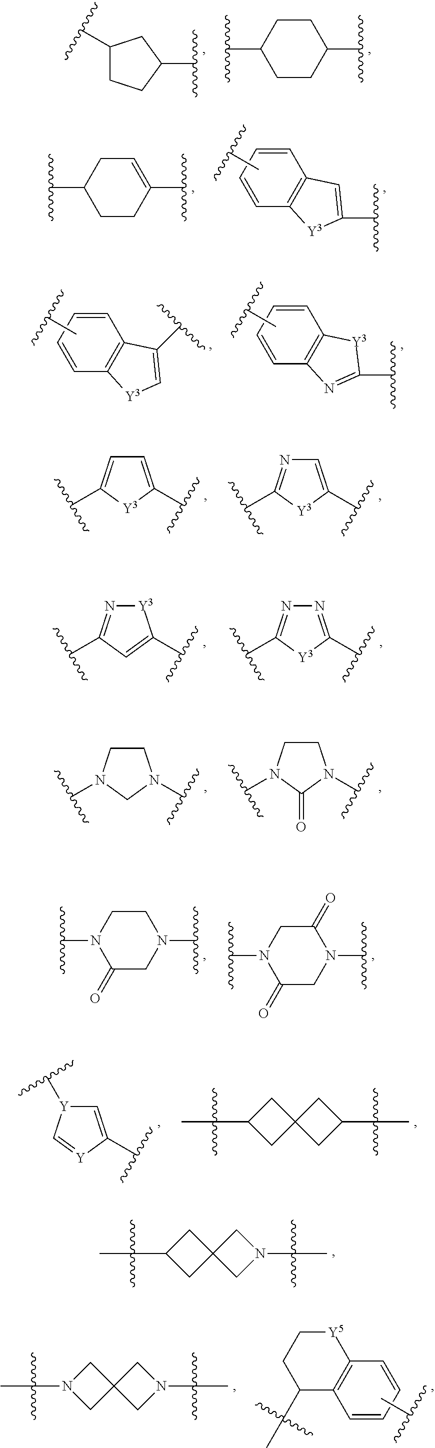 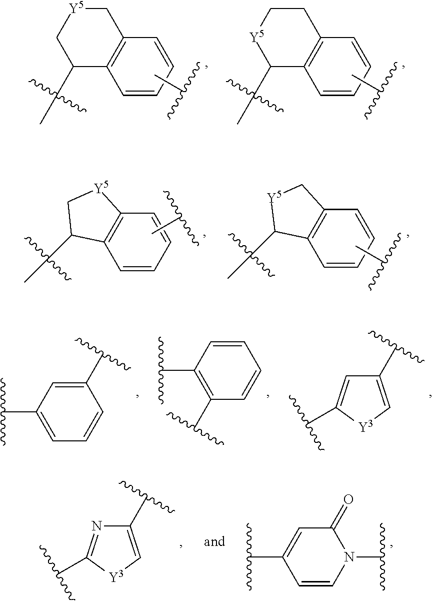
wherein each * is a point of attachment of B to L¹ or L³, wherein the rings in B are optionally substituted with one or more substituents selected from alkyl, amino, haloalkyl, halogen, hydroxy, alkoxy, haloalkoxy, cyano, sulfonyl or oxo;
G together with the atoms to which it is attached forms a ring system selected from 6-11 membered aryl, 5-11 membered heteroaryl, 4-11 membered heterocyclyl, and 4-11 membered carbocyclyl, wherein the ring system is optionally with one or more substituents selected from alkyl, amino, haloalkyl, halogen, or oxo; 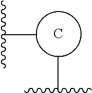
is selected from 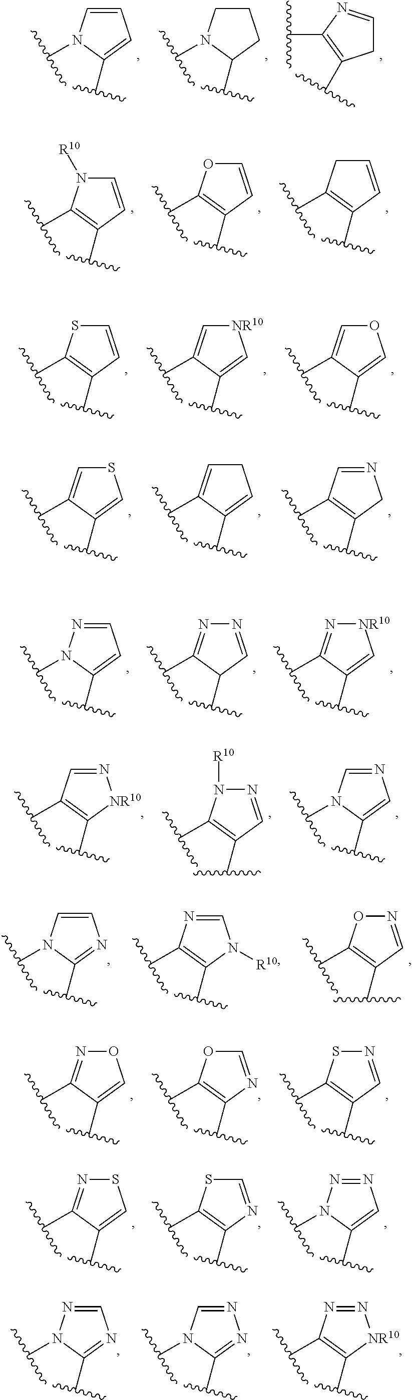 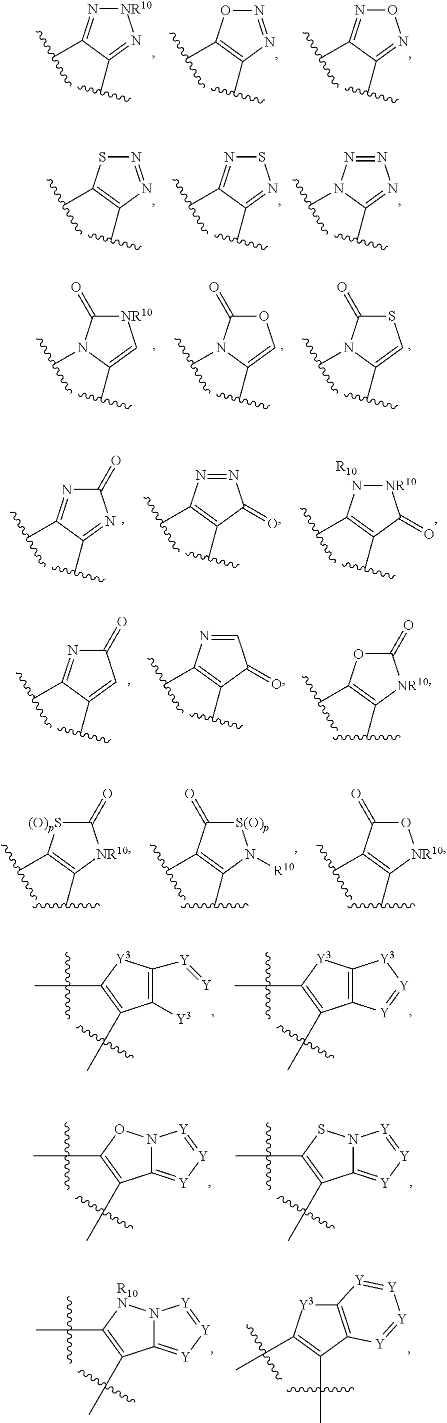 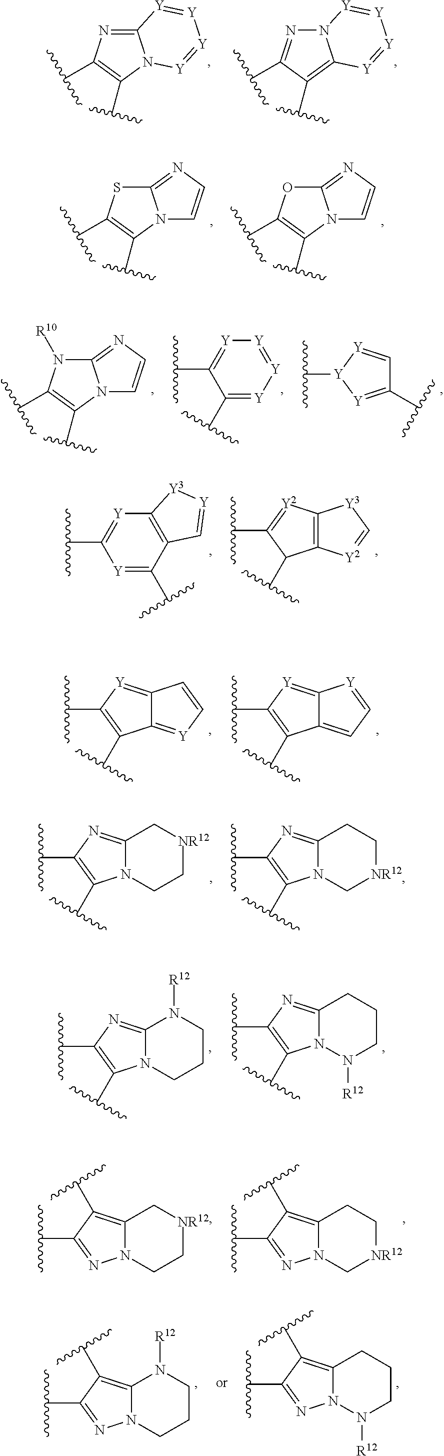
or optionally substituted variants thereof;
each Y² is independently selected from —CH═ or N;
each Y³ is independently selected from C(R⁶)₂, NR⁶, O or S;
each Y⁵ is independently selected from NR⁶, O or S;
each C₃₋₇ cycloalkyl, C₃₋₇ heterocyclyl, and 5-10 membered heteroaryl of R² or R³ is optionally substituted with one or more substituents selected from alkyl, haloalkyl, halogen, hydroxy, alkoxy, haloalkoxy, cyano, or oxo; and
each R¹² is independently selected from hydrogen; alkyl optionally substituted with one or more substituents selected from the group consisting of halogen, hydroxy and alkoxy; acyl; C-carboxy; C-amido; sulfinyl; sulfonyl; or S-sulfonamido.

474. The compound or pharmaceutically acceptable salt thereof of claim 471, wherein the compound of Formula (XII) is also represented by Formula (XIIa): 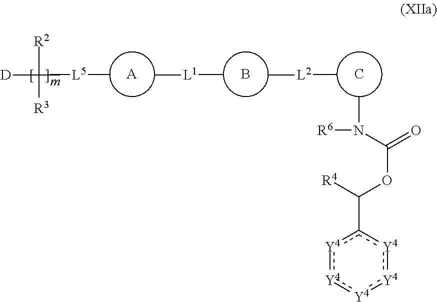
wherein A is selected from acetylene, 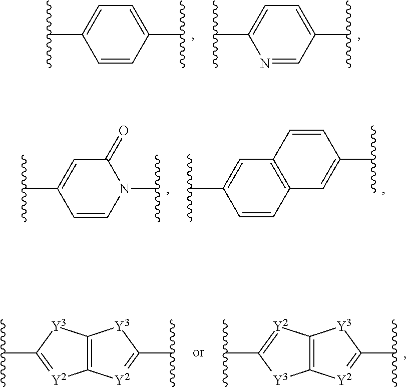
wherein the rings in A are unsubstituted or substituted with one or more substituents selected from alkyl, amino, haloalkyl, halogen, hydroxy, alkoxy, haloalkoxy, cyano, sulfonyl or oxo; and
B is selected from acetylene, 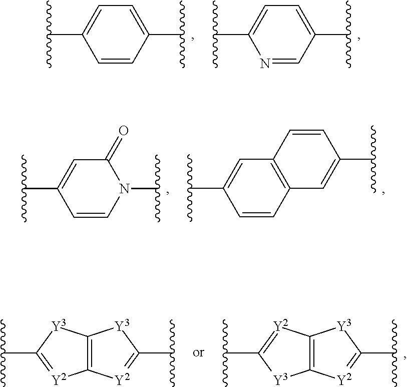
wherein the rings in B are unsubstituted or substituted with one or more substituents selected from alkyl, amino, haloalkyl, halogen, hydroxy, alkoxy, haloalkoxy, cyano, sulfonyl or oxo.

475. (canceled)

476. The compound or pharmaceutically acceptable salt thereof of claim 471, wherein D is —C(O)OR¹.

477. The compound or pharmaceutically acceptable salt thereof of claim 471, wherein both A and B are 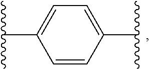 [SMILES: *c1ccc(*)cc1 ; ","]
each unsubstituted or substituted with one or more substituents selected from alkyl, amino, haloalkyl, halogen, hydroxy, alkoxy, haloalkoxy, cyano, or oxo.

478. The compound or pharmaceutically acceptable salt thereof of claim 471, wherein one of A or B is 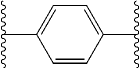 [SMILES: *c1ccc(*)cc1]
and the other A or B is selected from

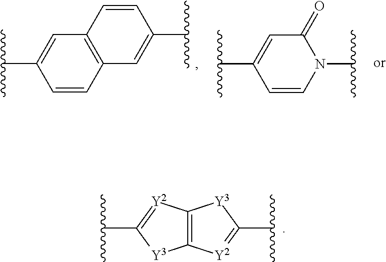

479. The compound or pharmaceutically acceptable salt thereof of claim 478, wherein A is 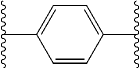 [SMILES: *c1ccc(*)cc1]
and B is selected from

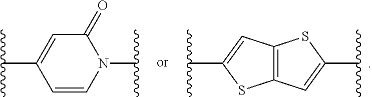 [SMILES: *c1ccn(*)c(=O)c1 ; "or" ; *c1cc2sc(*)cc2s1 ; "."]

480. The compound or pharmaceutically acceptable salt thereof of claim 478, wherein B is 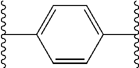 [SMILES: *c1ccc(*)cc1]
and A is selected from

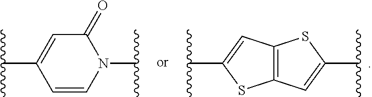 [SMILES: *c1ccn(*)c(=O)c1 ; "or" ; *c1cc2sc(*)cc2s1 ; "."]

481. The compound or pharmaceutically acceptable salt thereof of claim 471, wherein one of A or B is acetylene and the other A or B is selected from

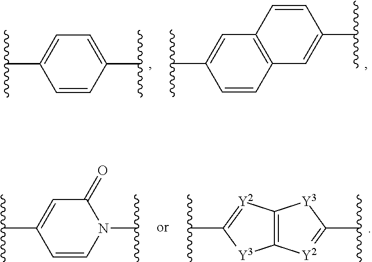

482. (canceled)

483. (canceled)

484. (canceled)

485. The compound or pharmaceutically acceptable salt thereof of claim 471, wherein C is selected from

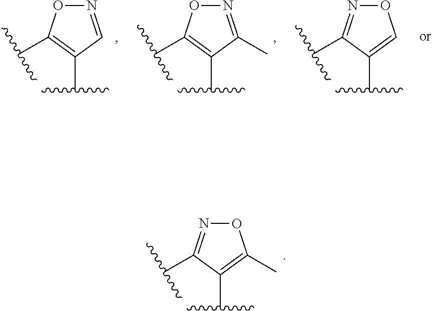

486. The compound or pharmaceutically acceptable salt thereof of claim 471, wherein C is selected from

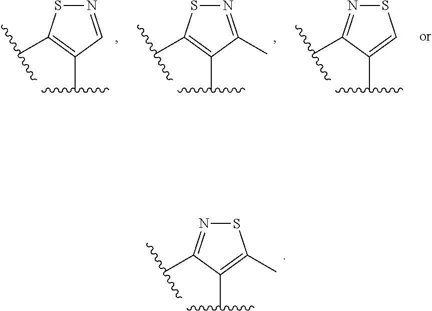

487. The compound or pharmaceutically acceptable salt thereof of claim 471, wherein C is selected from 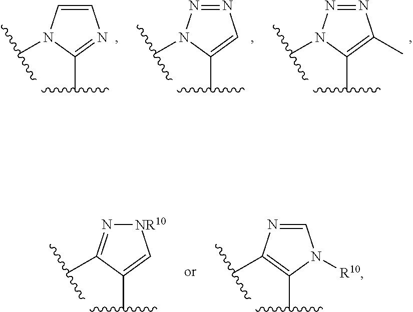
wherein R¹⁰ is selected from hydrogen, C₁₋₃ alkyl or C₃₋₆ cycloalkyl.

488. The compound or pharmaceutically acceptable salt thereof of claim 471, wherein C is selected from 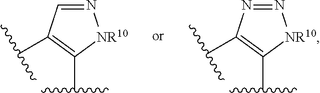
wherein R¹⁰ is selected from hydrogen, C₁₋₃ alkyl or C₃₋₆ cycloalkyl.

489. The compound or pharmaceutically acceptable salt thereof of claim 471, wherein m is 1.

490. The compound or pharmaceutically acceptable salt thereof of claim 471, wherein R¹ is hydrogen.

491. The compound or pharmaceutically acceptable salt thereof of claim 471, wherein at least one of R² and R³ is halogen or haloalkyl.

492. (canceled)

493. The compound or pharmaceutically acceptable salt thereof of claim 471, wherein R² is hydrogen and R³ is selected from optionally substituted cyclobutyl.

494. (canceled)

495. The compound or pharmaceutically acceptable salt thereof of claim 471, wherein R² is hydrogen and R³ is selected from optionally substituted oxetane.

496. (canceled)

497. The compound or pharmaceutically acceptable salt thereof of claim 471, wherein R² is hydrogen and R³ is selected from optionally substituted thiazolyl or optionally substituted oxazolyl.

498. (canceled)

499. The compound or pharmaceutically acceptable salt thereof of claim 471, wherein R² and R³ are joined together with the atom to which they are attached to form a C₃₋₆ cycloalkyl substituted by one or more halogen.

500. The compound or pharmaceutically acceptable salt thereof of claim 471, wherein R⁶ is hydrogen.

501. The compound or pharmaceutically acceptable salt thereof of claim 471, wherein each of L¹ and L² is a single bond.

502. (canceled)

503. The compound or pharmaceutically acceptable salt thereof of claim 471, wherein L⁵ is a single bond.

504. The compound or pharmaceutically acceptable salt thereof of claim 471, wherein L⁵ is a —O— linker.

505. The compound or pharmaceutically acceptable salt thereof of claim 471, wherein

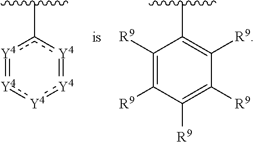

506. (canceled)

507. (canceled)

508. (canceled)

509. The compound or pharmaceutically acceptable salt thereof of claim 471, selected from compounds of Tables 12A and 12B, and pharmaceutically acceptable salt thereof.

510. The compound or pharmaceutically acceptable salt thereof of claim 471, selected from compounds IT123, IT136, IT150, IT151, IT172 and IT228 of Table 13.

511.-666. (canceled)

667. A pharmaceutical composition comprising an effective amount of a compound of claim 33, or a pharmaceutically acceptable salt thereof, and a pharmaceutically acceptable carrier, diluent, excipient or combination thereof.

668. A method for treating, preventing, reversing, halting, or slowing the progression of fibrosis, comprising administering an effective amount of a compound of claim 33, or a pharmaceutically acceptable salt thereof to a subject in need thereof.

669. (canceled)

670. The method of claim 668, wherein the fibrosis is selected from pulmonary fibrosis, dermal fibrosis, kidney fibrosis, or liver fibrosis.

671. The method of claim 670, wherein the pulmonary fibrosis is idiopathic pulmonary fibrosis.

672. (canceled)

673. The method of claim 668, wherein said compound, the pharmaceutical acceptable salt thereof, or the pharmaceutical composition is administered by inhalation.

674. (canceled)

675. (canceled)

676.-730. (canceled)

731. A pharmaceutical composition comprising an effective amount of a compound of claim 441, or a pharmaceutically acceptable salt thereof, and a pharmaceutically acceptable carrier, diluent, excipient or combination thereof.

732. A method for treating, preventing, reversing, halting, or slowing the progression of fibrosis, comprising administering an effective amount of a compound of claim 441, or a pharmaceutically acceptable salt thereof to a subject in need thereof.

733. The method of claim 732, wherein the fibrosis is selected from pulmonary fibrosis, dermal fibrosis, kidney fibrosis, or liver fibrosis.

734. The method of claim 733, wherein the pulmonary fibrosis is idiopathic pulmonary fibrosis.

735. The method of claim 732, wherein said compound, the pharmaceutical acceptable salt thereof, or the pharmaceutical composition is administered by inhalation.

736. A pharmaceutical composition comprising an effective amount of a compound of claim 471, or a pharmaceutically acceptable salt thereof, and a pharmaceutically acceptable carrier, diluent, excipient or combination thereof.

737. A method for treating, preventing, reversing, halting, or slowing the progression of fibrosis, comprising administering an effective amount of a compound of claim 471, or a pharmaceutically acceptable salt thereof to a subject in need thereof.

738. The method of claim 737, wherein the fibrosis is selected from pulmonary fibrosis, dermal fibrosis, kidney fibrosis, or liver fibrosis.

739. The method of claim 738, wherein the pulmonary fibrosis is idiopathic pulmonary fibrosis.

740. The method of claim 737, wherein said compound, the pharmaceutical acceptable salt thereof, or the pharmaceutical composition is administered by inhalation.
